SEQUENCE LISTING (6 sequences)

SEQ ID NO: 1 - DNA, 528 bp, Homo sapiens
  CDS (1)..(528)
gtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaag

SEQ ID NO: 2 - protein, 176 aa, Homo sapiens
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREK

SEQ ID NO: 3 - DNA, 516 bp, Homo sapiens
  CDS (1)..(516)
gtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagct

SEQ ID NO: 4 - protein, 172 aa, Homo sapiens
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKA

SEQ ID NO: 5 - DNA, 23 bp, Artificial sequence
  Synthetic Primer
agctccgcgtgagaagtaataag

SEQ ID NO: 6 - DNA, 23 bp, Artificial sequence
  Synthetic Primer
gatccttattacttctcacgcgg

(19) United States
(12) Patent Application Publication
(10) Pub. No.: US 2004/0033527 A1
(43) Pub. Date: Feb. 19, 2004

(54) Methods of using a three-dimensional model of a Fc epsilon receptor alpha chain

(76) Inventors: Theodore S. Jardetzky, Chicago, IL (US); Scott Clayton Garman, Evanston, IL (US); Jean-Pierre Kinet, Lexington, MA (US)

Correspondence Address: HESKA CORPORATION, INTELLECTUAL PROPERTY DEPT., 1613 PROSPECT PARKWAY, FORT COLLINS, CO, 80525, US

(21) Appl. No.: 10/293,992

(22) Filed: Nov. 13, 2002

Related U.S. Application Data
(62) Division of application No. 09/434,193, filed on Nov. 4, 1999.
(60) Provisional application No. 60/107,219, filed on Nov. 5, 1998.

(51) Int. Cl. G01N 33/53; G06G 7/48; G06G 7/58

(52) U.S. Cl. 435/7.1; 703/11

(57) ABSTRACT

The present invention includes three-dimensional models of antibody receptor proteins, such as FcεRIα proteins, and methods to produce such models. The present invention also includes muteins having increased stability and/or antibody binding activity, as well as methods to produce such muteins, preferably using information derived from three-dimensional models of the present invention. Also included are nucleic acid sequences encoding muteins of the present invention and use of those sequences to produce such muteins. Also included is the use of the model to identify compounds that inhibit the binding of an antibody receptor protein to an antibody. The present invention also includes uses of such muteins and inhibitory compounds, for example, in methods to diagnose and protect animals from allergy and other abnormal immune responses.

### CROSS-REFERENCE TO RELATED APPLICATION

This application claims priority to U.S. Provisional Patent Application Serial No. 60/107,219, filed Nov. 5, 1998. The patent application referred to in this section is incorporated by reference herein in its entirety.

This invention was made at least in part with government support under NIH Grant No. RO1 AI38972, awarded by the National Institutes of Health to Northwestern University. The government has certain rights to this invention.

### FIELD OF THE INVENTION

The present invention relates to a crystal and a three-dimensional (3-D) model of a Fc epsilon receptor alpha chain as well as to the use of that model to produce muteins and inhibitors useful in the diagnosis and treatment of allergy and the regulation of other immune responses in an animal.

### BACKGROUND OF THE INVENTION

Antibody Fc-receptors (FcRs) play an important role in the immune response by coupling the specificity of secreted antibodies to a variety of cells of the immune system. A number of cell types, including macrophages, mast cells, eosinophils, and basophils, express membrane-bound FcRs at their surfaces. The binding of antibodies to FcRs provides antigen-specificity to these cells, which upon activation release further cell-specific mediators of the immune response, such as interleukins, initiators of inflammation, leukotrienes, prostaglandins, histamines, or cytotoxic proteins. The adoptive specificity of the FcRs allows a combinatorial approach to pathogen elimination, by coupling the diversity of antibody antigen-recognition sites to the variety of cell-types expressing these receptors.

FcR-initiated mechanisms are important in normal immunity to infectious disease as well as in allergies, antibody-mediated tumor recognition, autoimmune diseases, and other diseases in which immune responses are abnormal (i.e., not regulated). Recent experiments with transgenic mice have demonstrated that the FcRs control key steps in the immune response, including antibody-directed cellular cytotoxicity and inflammatory cascades associated with the formation of immune complexes; see, for example, Ravetch et al., 1998, Annu Rev Immunolo 16, 421-432. Receptors that bind IgG (FcgRI, FcgRII, and FcgRIII, known collectively as FcgRs) mediate a variety of inflammatory reactions, regulate B-cell activation, and also trigger hypersensitivity reactions. The high affinity Fc epsilon receptor (also known as the IgE receptor or FceRI) is associated with the activation of mast cells and the triggering of allergic reactions and anaphylactic shock. Knockout mice for the FceRI alpha chain (FcεRIα) are unable to mount IgE-mediated anaphylaxis (see for example, Dombrowicz et al., 1993, Cell 75, 969-976), although FcgRs are still able to activate mast cells (see, for example, Dombrowicz et al., 1997, J. Clin. Invest. 99, 915-925; Oettgen et al., 1994, Nature 370, 367-370). FceRI has also been shown to trigger anti-parasitic reactions from platelets and eosinophils as well as deliver antigen into the MHC class II presentation pathway for the activation of T cells; see, for example, Gounni et al., 1994, Nature 367, 183-186; Joseph et al., 1997, Eur. J. Immunol. 27, 2212-2218; Maurer et al., 1998, J. Immunol. 161, 2731-2739. The b-subunit of FceRI has been associated with asthma in genetic studies; see, for example, Hill et al., 1996, Hum. Mol. Genet. 5, 959-962; Hill et al., 1995, Bmj 311, 776-779; Kim et al., 1998, Curr. Opin. Pulm. Med. 4, 46-48; Mao et al., 1998, Clin. Genet. 53, 54-56; Shirakawa et al., 1994, Nat. Genet. 7, 125-129. A significant fraction of the population (˜20%) may be affected by allergies, and this century has seen a substantial increase in asthma. Since IgE binding to FceRI is a requisite event in the reaction to different allergens, therapeutic strategies aimed at inhibiting FceRI could provide a useful treatment for these diseases. For example, monoclonal antibodies that target IgE and block receptor binding have shown therapeutic potential; see, for example, Heusser et al., 1997, Curr. Opin. Immunol. 9, 805-813.

FceRI is found as a tetrameric (abg₂) or trimeric (ag₂) membrane bound receptor on the surface of mast cells, basophils, eosinophils, langerhans cells and platelets. The alpha chain, also referred to as FcεRIα, of FceRI binds IgE molecules with high affinity (K_{D} of about 10⁻⁹ to 10⁻¹⁰ moles/liter (M)), and can be secreted as a 172-amino acid soluble, IgE-binding fragment by the introduction of a stop codon before the single C-terminal transmembrane anchor; see, for example, Blank et al., 1991, E. J. Biol. Chem. 266, 2639-2646, which describes the secretion of a soluble IgE-binding fragment of 172 amino acids. The extracellular domains of the human FcεRIα protein belong to the immunoglobulin (Ig) superfamily and contain seven N-linked glycosylation sites. Glycosylation of FcεRIα affects the secretion and stability of the receptor, but is not required for IgE-binding; see, for example, LaCroix et al., 1993, Mol. Immunol. 30, 321-330; Letourneur et al., 1995, J. Biol. Chem. 270, 8249-8256; Robertson, 1993, J. Biol. Chem. 268, 12736-12743; Scarselli et al., 1993, FEBS Lett 329, 223-226. The beta and gamma chains of FceRI are signal transduction modules.

Prior investigators have disclosed the nucleic acid sequence for human FcεRIα; see, for example, U.S. Pat. No. 4,962,035, by Leder, issued Oct. 9, 1990; U.S. Pat. No. 5,639,660, by Kinet et al., issued Jun. 17, 1997; Kochan et al., 1988, Nucleic Acids Res. 16, 3584; Shimizu et al., 1988, Proc. Natl. Acad. Sci. USA 85, 1907-1911; and Pang et al., 1993, J. Immunol. 151, 6166-6174. Nucleic acid sequences have also been reported for the human FCεRI beta and gamma chains; see, respectively, Kuster et al., 1992, J. Biol. Chem. 267, 12782-12787; Kuster et al., 1990, J. Biol. Chem. 265, 6448-6452. Nucleic acid sequences have also been reported for nucleic acid molecules encoding canine FcεRIα, murine FcεRIα, rat FcεRIα, feline FcεRIα and equine FcεRIα proteins; see, respectively, GenBank™ accession number D16413; Swiss-Prot accession number P20489 (represents encoded protein sequence); GenBank accession number J03606; PCT Publication No. WO 98/27208, by Frank et al., published Jun. 25, 1998, referred to herein as WO 98/27208; and PCT Publication No. WO 99/38974, by Weber et al., published Aug. 5, 1999, referred to herein as WO 99/38974. In addition, methods to detect IgE antibodies using a FcεRIα protein have been reported in PCT Publication No. WO 98/23964, by Frank et al., published Jun. 4, 1998, referred to herein as WO 98/23964; WO 98/27208, ibid.; PCT Publication No. WO 98/45707, by Frank et al., published Oct. 15, 1998, referred to herein as WO 98/45707; and WO 99/38974, ibid. WO 98/23964, WO 98/27208, WO 98/45707 and WO 99/38974 are each incorporated by reference herein in its entirety.

There have been several reports of the use of mutagenesis and swapping techniques to attempt to identify amino acids of either FcεRIα or IgE involved in the binding of (i.e., interaction between) those respective proteins, reports attempting to model FcεRIα proteins based on homology to other Ig-superfamily members, and reports that identify compounds that apparently inhibit such binding; see, for example, Cook et al., 1997, Biochemistry 36, 15579-15588; Hulett et al., 1994, J. Biol. Chem. 269, 15287-15293; Hulett et al., 1995, J. Biol. Chem 270, 21188-21194; Mallamaci et al., 1993, J. Biol. Chem. 268, 22076-22083; Robertson, 1993, ibid.; Scarselli et al., 1993, ibid. McDonnell et al., 1997, Biochem. Soc. Trans. 25, 387-392; McDonnell et al., 1996, Nat. Struc. Biol. 3, 419-426; PCT Publication No. WO 97/40033, by Cheng et al., published Oct. 30, 1997; U.S. Pat. No. 5,180,805, by Gould et al, issued Jan. 19, 1993; U.S. Pat. No. 5,693,758, by Gould et al., issued Dec. 2, 1997; PCT Publication No. WO 96/01643, by Gould et al., published Jan. 25, 1996; PCT Publication No. WO 95/14779, by Gould et al., published Jun. 1, 1995. None of these references, however, describe isolated crystals of FcεRIα proteins or 3-D models derived from crystals.

Despite what is known about FcRs and their interaction with antibodies, there remains a need for FcRs with improved characteristics, such as enhanced affinity for antibodies, altered substrate specificity, increased stability, and increased solubility for use in diagnosis, treatment and prevention of allergy and other abnormal immune responses. Also needed for safe and efficacious compounds to prevent or treat allergy and to regulate other immune responses in an animal.

### SUMMARY OF THE INVENTION

The present invention includes isolated crystals of the extracellular domains of antibody receptor proteins (FcRs), three-dimensional (3-D) models of such crystals and modifications of such models. The present invention also includes compounds that inhibit the ability of FcRs to bind to antibodies as well as FcR muteins and other modified FcRs. Also included in the present invention are methods to produce and use such crystals, models, inhibitory compounds, muteins, and other modified proteins. As such, the present invention includes FcRs with improved functions such as increased stability, increased affinity for an Fc domain of an antibody, altered substrate specificity, and increased solubility, including but not limited to reduced aggregation. Such proteins, also referred to as muteins, are useful to detect allergy and other immune response abnormalities as well as to protect an animal from such abnormalities. The present invention also provides safe and efficacious inhibitory compounds to protect (e.g., prevent, treat, reduce the consequences of) an animal from allergy and to regulate other immune responses in an animal.

The present invention includes a 3-D model of an extracellular domain of a human high affinity Fc epsilon receptor alpha chain (FcεRIα) protein, wherein the model substantially represents the atomic coordinates specified in Table 1, Table 5, Table 6, Table 7 or Table 8. The present invention also includes a 3-D model comprising a modification of a model substantially representing the atomic coordinates specified in Table 1, Table 5, Table 6, Table 7 or Table 8. Also included in the present invention are methods to produce such models.

The present invention also includes an isolated crystal of an extracellular domain of a FcεRIα protein and methods to produce such a crystal.

The present invention also includes an isolated FcεRIα protein consisting of SEQ ID NO: 2 or of SEQ ID NO: 4 except that the isoleucine at position 170 is replaced by a cysteine, as well as a protein that is structurally homologous to either such protein. Also included are nucleic acid molecules encoding such proteins, recombinant molecules and recombinant cells including such proteins, and methods to produce such proteins.

The present invention includes a method to identify a compound that inhibits the binding between an IgE antibody and a FcεRIα protein. The method includes the step of using a 3-D model of an extracellular domain of a human FcεRIα protein to identify the compound. Such a model substantially represents the atomic coordinates specified in Table 1, Table 5, Table 6, Table 7 or Table 8. Also included in the present invention are inhibitory compounds identified using such a method. Also included are therapeutic compositions that include such inhibitory compounds and methods to use such therapeutic compositions to protect an animal from allergy or to regulate other immune responses (e.g., protect an animal from other abnormal immune responses).

The present invention also includes a mutein that binds to a Fc domain of an antibody. Such a mutein has an improved function compared to a protein that includes SEQ ID NO: 2 or SEQ ID NO: 4. Examples of such an improved function include increased stability, increased affinity for an Fc domain of an antibody, altered substrate specificity, decreased aggregation, and increased solubility. Such a mutein is produced by a method that includes the following steps: (a) analyzing a 3-D model substantially representing the atomic coordinates specified in Table 1, Table 5, Table 6, Table 7 or Table 8 to identify at least one amino acid of the protein represented by the model which if replaced by a specified amino acid would effect an improved function of the protein; and (b) replacing the identified amino acid(s) to produce the mutein having such an improved function. The present invention also includes a mutein having an improved function compared to an unmodified FcεRIα protein, wherein the amino acid sequence of the mutein differs in at least one position from the amino acid sequence of the unmodified protein. Such a position(s) is in at least one of the following regions: a crystal contact cluster, a tryptophan-containing hydrophobic ridge, a FG loop in D2, a D1D2 interface, a cleft between D1 and D2, a domain 1, a domain 2, a hydrophobic core, a A′B loop of D1, a EF loop of D1, a BC loop of D2, a C strand of D2, a CC′ loop of D2, C′E loop of D2, a strand of D2, the amino terminal five residues of the protein, the carboxyl terminal five residues of the protein, and N-linked glycosylation sites.

Also included are muteins that are chemically modified FcεRIα proteins. Also included are nucleic acid molecules that encode muteins of the present invention, recombinant molecules and recombinant cells including such nucleic acid molecules and methods to produce such muteins. Also included are diagnostic reagents and diagnostic kits including such muteins, therapeutic compositions including such muteins, and methods to detect or protect an animal from allergy or other abnormal immune responses.

The present invention also includes a method to improve a function of a FcεRIα protein which includes the steps of: (a) analyzing a 3-D model of an extracellular domain of a human high affinity FcεRIα protein substantially representing the atomic coordinates specified in Table 1, Table 5, Table 6, Table 7 or Table 8, to identify at least one amino acid of the protein which if replaced by a specified amino acid improves at least one of the functions of the protein; and (b) replacing the identified amino acid(s) to produce a mutein having at least one of the improved functions.

### BRIEF DESCRIPTION OF THE FIGURES

FIG. 1 depicts electron density maps and overall structure of a human FcεRIα model. (A) The 3.0 angstrom experimental electron density map, calculated using the MIRAS phases followed by density modification with the program DM is shown along with a refined model for human FcεRIα. The density is contoured at 1.4σ for residues 147-153. (B) Electron density for carbohydrate moieties linked to N42. The |2Fo-Fc| electron density map, contoured at 1σ, was calculated to 2.4 angstroms using combined MIRAS and model phases (prior to inclusion of carbohydrate in the model). Two N-acetylglucosamines and a mannose moiety were built into the density as shown.

FIG. 2 depicts a ribbon diagram of a human FcεRIα model showing the positions of the disulfides and the FG loop in domain 2 (D2) that is implicated in receptor specificity. Domain 1 (D1) is shown to the right and D2 is shown to the left.

FIG. 3 depicts a topology diagram of the two domains of a human FcεRIα model showing the hydrogen-bonding patterns of the beta sheet structure. The short stretch of parallel beta-sheet in D1 and D2 caused by the cross-over of the A strand is highlighted. Note that the FG strands of D2 are longer than those of D1, contributing to the prominence of the D2-FG loop.

FIG. 4 demonstrates that a human FcεRIα model has a novel tertiary arrangement of tandem Ig domains.

FIG. 5 depicts sequence alignments of human FcRs. The secondary structure of the two domains is indicated with labeled bars above those residues which form beta-sheet in FcεRI. Below the sequences, carbohydrate attachment sites found in seventeen different FcR sequences are indicated with a (+). This analysis is based on the seven human receptors shown and the non-human receptors listed in Table 4.

FIG. 6 depicts the four surface-exposed tryptophans at the top of the D2 domain of a human FcεRIα model that are implicated in IgE binding.

FIG. 7 depicts residues in the D2 FG loop and D1 E strand of a human FcεRIα model that are highly variable in human FcR sequences. The residues in the D2-FG loop have been directly implicated in IgE binding. The residues in the D1 E strand and the D1 A′B loop are located near the top of the D2 domain and could form part of an extended IgE-binding surface between the two domains.

FIG. 8 depicts a juxtaposition of a human FcεRIα model with a model for the intact IgE antibody structure. The insertion of the Cε2 domains in the IgE molecule are indicated by dotted lines. The FcεRIα protein is shown relative to the mast cell membrane near the top of the Cε3 domains that bind to the receptor.

### DETAILED DESCRIPTION OF THE INVENTION

The present invention includes isolated crystals of the extracellular domains of FcRs, 3-D models of such crystals and modifications of such models. The present invention also includes compounds that inhibit the ability of FcRs to bind to antibodies as well as muteins and other modified FcRs. Also included in the present invention are methods to produce and use such crystals, models, inhibitory compounds, muteins, and other modified proteins.

The present invention includes an isolated crystal of an extracellular domain of a high affinity Fc epsilon receptor alpha chain (FcεRIα), a 3-D model of such a crystal and a modification of such a model. As used herein, the term “a” entity or “an” entity refers to one or more of that entity; for example, a crystal or a model refers to one or more crystals or models, respectively. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures, or combinations, of two or more of the compounds.

As used herein, an extracellular domain of a FcεRIα protein is the portion of the FceRI alpha chain that is exposed to the environment outside the cell and that binds to the Fc domain of an IgE antibody. Such an extracellular domain can be (a) a complete extracellular domain which is a domain that extends from the first amino acid of a mature FceRI alpha chain through the last amino acid prior to the start of the transmembrane region or a domain that is functionally equivalent, in that such a domain includes a D1 and D2 domain, displays a similar affinity for the IgE antibody to which such an FcεRIα protein naturally binds, and produces crystals having sufficient quality to enable structure determination, or (b) a fragment of any of the extracellular domains of (a), wherein the fragment retains its ability to bind to the Fc domain of an antibody. As used herein, the terms binding to an antibody and binding to the Fc domain (i.e., constant region) of an antibody can be used interchangeably since it is recognized that a FcR binds to the Fc domain of an antibody. A FcR (i.e., a protein that can bind to an antibody), such as a FcεRIα protein, can be a full-length FcR (e.g., a full-length FceRI alpha chain), or any fragment thereof, wherein the fragment binds to an antibody. Similarly an antibody, or an Fc domain thereof, can be a full-length antibody, or full-length Fc domain thereof, or any fragment thereof that binds to a FcR. Preferably a FcR binds to an antibody with an affinity (K_{A}) of at least about 10⁸ liters/mole (M⁻¹), more preferably of at least about 10⁹ M⁻¹, and even more preferably of at least about 10¹⁰ M⁻¹.

The present invention is surprising in several aspects. For example, this is the first report of an isolated crystal of an extracellular domain of a FcεRIα protein, and in particular of an isolated crystal of sufficient quality that a crystal structure, i.e., a 3-D model, could be derived therefrom. The inventors believe that this protein also represents the most highly glycosylated protein for which a crystal and a 3-D model have been reported to date. Not only does glycosylation interfere with protein crystal formation but it also is difficult to consistently produce recombinant proteins having a uniform glycosylation pattern. Generation of such a crystal was very difficult and non-obvious and has been attempted by others without success. The inventors tried many approaches before discovering that a preferred FcεRIα protein from which to make a useful crystal is a FcεRIα protein that consists of amino acids 1 through 176 of the mature human FcεRIα protein. This protein is denoted herein as PhFcεRIα₁₋₁₇₆, or the hFcεRIα₁₋₁₇₆ protein, and has an amino acid sequence denoted herein as SEQ ID NO: 2. An example of a nucleotide acid molecule encoding PhFcεRIα₁₋₁₇₆ is referred to herein as nhFcεRIα₁₋₅₂₈, the nucleic acid sequence of which is denoted herein as SEQ ID NO: 1. It was also discovered that better crystals are generated when PhFcεRIα₁₋₁₇₆ is produced in insect cells, using a method such as that described in the Examples. Determination of the crystal structure of PhFcεRIα₁₋₁₇₆ produced in Trichoplusia ni (Hi-5) cells resulted in a 3-D model that substantially represents the atomic coordinates specified in Table 1, referred to herein as form M1. Amino acids are represented herein by their standard three or one letter codes; see, for example, Sambrook et al., Molecular Cloning: A Laboratory Manual, Cold Spring Harbor Labs Press, 1989, which is incorporated herein by reference in its entirety. Prior to obtaining a crystal of sufficient quality to solve its crystal structure using insect-cell produced PhFcεRIα₁₋₁₇₆, a number of other proteins were tried, including a FcεRIα protein spanning from amino acid 1 through 171 of SEQ ID NO: 2 produced in Pichia pastoris, and FcεRIα proteins spanning from amino acid 1 through 172 of SEQ ID NO: 2 produced in Chinese hamster ovary cells, Trichoplusia ni cells, and Spodoptera frugiperda cells without success. Without being bound by theory, it is believed that PhFcεRIα₁₋₁₇₆ was a better candidate because it apparently represents a complete extracellular domain. Based on the 3-D model of PhFcεRIα₁₋₁₇₆, the inventors believe, without being bound by theory, that the amino acid at position 172 is important in the structure determination and that, in order to form a crystal of sufficient quality to determine the first 3-D model of a FcεRIα protein, at least one additional amino acid was required carboxyl-terminal to that at position 172; the inventors further believe that an optimal protein would span from the amino acid at position 3 through the amino acid at position 174 of SEQ ID NO: 2. It should be noted, however, that having solved the crystal structure of a first FcεRIα protein enables the solving of crystal structures of additional FcεRIα proteins as well as of additional FcRs in general. For example, the crystal structures of two additional crystals cited in the Examples can be solved using a combination of X-ray diffraction data of the crystals per se and information derived from the 3-D model of PhFcεRIα₁₋₁₇₆. The examples also describe the solution of an additional four crystal structures using such information, namely the examples present 3-D models of: (a) a human FcεRIα protein spanning amino acids 1-172 of SEQ ID NO: 2 (i.e., PhFcεRIα₁₋₁₇₂, the amino acid sequence of which is represented herein as SEQ ID NO: 4) expressed in lec1 Chinese hamster ovary (CHO) cells, the structural form being referred to herein as Form T1; (b) a second structural form of PhFcεRIα₁₋₁₇₂ produced in lec1 CHO cells, referred to herein as Form T2; (c) a second structural form of a PhFcεRIα₁₋₁₇₆ protein expressed in T. ni (Hi5) cells, referred to herein as Form M2; and (d) a PhFcεRIα₁₋₁₇₂ protein in which the isoleucine at position 170 of SED ID NO: 4 is replaced with a cysteine, expressed in Sf9 insect cells, a structural form referred to herein as H1. The atomic coordinates of the crystal structural forms T1, T2, M2 and H1 are presented, respectively, in Tables 5, 6, 7, and 8.

The 3-D model of the hFcεRIα₁₋₁₇₆ protein form M1 is also very surprising in view of the knowledge of the structure of proteins containing immunoglobulin domains, herein also referred to as Ig domains. The most striking differences, which are described in greater detail below, include, but are not limited to: domain 1 (D1) and domain 2 (D2) of the model of PhFcεRIα₁₋₁₇₆ are much smaller than known Ig domains; the packing and orientation of D1 and D2 of the hFcεRIα₁₋₁₇₆ protein are significantly different from known Ig domain-containing proteins in that, for example, the bend angle between D1 and D2 of the PhFcεRIα₁₋₁₇₆ structure is much more acute than for other proteins, the relative rotational orientation of the two domains is much different, D1 and D2 of PhFcεRIα₁₋₁₇₆ form an unusual interface and cleft, D1 and D2 of PhFcεRIα₁₋₁₇₆ are antiparallel, the presence of a hydrophobic surface on the two faces of the model of PhFcεRIα₁₋₁₇₆ which appear to be nearby or directly involved in binding to IgE antibodies; the FG loop of D2 of PhFcεRIα₁₋₁₇₆, also apparently involved in binding to IgE antibodies, projects much more significantly above the D2 domain than is seen for known D2-containing proteins; and the interruption in structure between strands A and A′ in D1 which apparently leads to interaction between the two domains. It is to be noted that although most known Ig domain pairs which are parallel, some Ig domains are antiparallel (e.g., hemolin) but the domain:domain orientation and specifics of packing of those domains are very different from the orientation and packing of PhFcεRIα₁₋₁₇₆. It is also surprising that the model of the hFcεRIα₁₋₁₇₆ protein predicts that an IgE antibody interacts with D1 as well as D2 in view of the mutagenesis analysis studies conducted to date all of which have only identified mutations in D2 that lead to decreased, or increased, binding between a FcεRIα protein and an IgE antibody. As such, a model of the present invention is necessary for proper interpretation and refinement of mutagenesis and region swapping studies that have been reported. Such a model for the first time permits the differentiation between amino acids directly or indirectly influencing binding of IgE to FcεRIα and demonstrates where amino acids and amino acid segments identified in mutagenesis and swapping studies are positioned on the protein. It is to be noted that the 3-D models of FcεRIα crystal structure forms T1, T2, M2 and H1 are quite similar to that of form M1, with the following differences. The principal differences in the structures from the various crystal forms occur in the BC loop in domain 1 (the “30 loop”), the C′ strand in domain 2 (the “130 region”) and the carbohydrate sites. There are also smaller differences in the termini of the structures and the FG loop in domain 1 (the “72 loop”). These differences are described in more detail in the Examples.

One embodiment of the present invention is an isolated crystal of an extracellular domain of a FcεRIα protein. As used herein, an isolated crystal is a crystal of a protein that has been produced in a laboratory; that is, an isolated crystal is produced by an individual and is not an object found in situ in nature. It is appreciated by those skilled in the art that there are a variety of techniques to produce crystals including, but not limited to, vapor diffusion using a hanging or sitting drop methodology, vapor diffusion under oil, and batch methods; see, for example, Ducruix et al., eds., 1991, Crystallization of nucleic acids and proteins; A practical approach, Oxford University Press, and Wyckoff et al., eds., 1985, Methods in Enzymology 11, 49-185; each reference is incorporated by reference herein in its entirety. It is also to be appreciated that crystallization conditions can be adjusted depending on a protein's inherent characteristics as well as on a protein's concentration in a solution and that a variety of precipitants can be added to a protein solution in order to effect crystallization; such precipitants are known to those skilled in the art. In a preferred embodiment, a crystal of a FcεRIα protein is produced in a solution by adding a precipitant such as polyethylene glycol (PEG) or PEG monomethylether. In a particularly preferred embodiment, the precipitant PEG is added to a solution to achieve a final concentration of from about 10 percent (%) to about 40%, preferably from about 12% to about 32% PEG per volume solution. It is also to be noted that a FcεRIα protein used to produce a crystal can be produced by a variety of methods, including purification of a native protein, chemical synthesis of a protein, or recombinant production of a protein. Although a number of cell types can be used to recombinantly produce such a protein, insect cells, such as, but not limited to Trichoplusia ni and Spodoptera frugiperda, are preferred, with Trichoplusia ni cells being more preferred. Also preferred are Chinese hamster ovary cells. Additional methods to produce proteins are disclosed below.

Isolated crystals of the present invention can include heavy atom derivatives, such as, but not limited to, gold, platinum, mercury, selenium, and lead. Such heavy atoms can be introduced randomly or introduced in a manner based on knowledge of 3-D models of the present invention. Additional crystals of the present invention are not derivatized. In one embodiment, an isolated crystal of the present invention is a co-crystal of a FcεRIα protein bound to a Fc domain of an IgE antibody. In another embodiment, an isolated crystal of the present invention is a co-crystal of a FcεRIα protein and a compound that inhibits the binding of a FcεRIα protein to a Fc domain of an IgE antibody. Additional crystals of the present invention include crystals produced from proteins that are muteins of the present invention or other proteins that are represented by a 3-D model of the present invention.

An isolated crystal of the present invention can be the crystal of any suitable extracellular domain of a FcεRIα protein. Suitable FcεRIα proteins include mammalian FcεRIα proteins, with human, canine, feline, equine, rat and murine FcεRIα proteins being preferred, and human FcεRIα proteins being even more preferred. A preferred crystal of the present invention diffracts X-rays to a resolution of about 4.0 angstroms or higher (i.e., lower number meaning higher resolution), with resolutions of about 3.5 angstroms or higher, about 3 angstroms or higher, about 2.5 angstroms or higher, about 2 angstroms or higher, about 1.5 angstroms or higher, and about 1 angstrom or higher being increasingly more preferred. It is appreciated, however, that additional crystals of lower resolutions can have utility in discerning overall topology of the structures, e.g., location of a binding site or where a molecule binds to a receptor. A particularly preferred isolated crystal of the present invention has the amino acid sequence SEQ ID NO: 2, amino acid sequence SEQ ID NO: 4, or a sequence essentially equivalent that represents an extracellular domain of another mammalian FcεRIα protein. SEQ ID NO: 4 is the amino acid sequence of a protein consisting of the first 172 residues of a mature human FcεRIα protein denoted herein as PhFcεRIα₁₋₁₇₂; i.e., SEQ ID NO: 4 spans from amino acid residue 1 through amino acid residue 172 of SEQ ID NO: 2. An example of a nucleotide acid molecule encoding PhFcεRIα₁₋₁₇₂ is referred to herein as nhFcεRIα₁₋₅₁₆, the nucleic acid sequence of which is denoted herein as SEQ ID NO: 3. Preferred are crystals that belong to monoclinic space group C2 or monoclinic space group P6122. Particularly preferred crystals include: a crystal of PhFcεRIα₁₋₁₇₆ that belongs to monoclinic space group C2, has cell dimensions of 88.6 angstroms×69.6 angstroms×49.3 angstroms, alpha=gamma=90.0 degrees, beta=116.69 degrees, and diffracts X-rays to a resolution of about 2.4 angstroms (form M1); a crystal of PhFcεRIα₁₋₁₇₆ that belongs to monoclinic space group C2, has cell dimensions of 136.02 angstroms×75.01 angstroms×79.28 angstroms, alpha=gamma=90 degrees, beta=117.8 degrees, and diffracts X-rays to a resolution of about 3.0 angstroms; and a crystal of PhFcεRIα₁₋₁₇₂ that belongs to monoclinic space group P6122, has cell dimensions of 58 angstroms×58 angstroms×226 angstroms, alpha=beta=90 degrees, gamma=120 degrees, and diffracts X-rays to a resolution of about 3.2 angstroms. Also preferred crystals include: a crystal of PhFcεRIα₁₋₁₇₂ that belongs to tetragonal space group P4₃, has cell dimensions of 145.08 angstroms×145.08 angstroms×62.74 angstroms, alpha=beta=gamma=90.0 degrees, and diffracts X-rays to a resolution of about 3.1 angstroms (form T1); a crystal of PhFcεRIα₁₋₁₇₂ that belongs to tetragonal space group P4₃, has cell dimensions of 150.50 angstroms×150.50 angstroms×74.18 angstroms, alpha=beta=gamma=90.0 degrees, and diffracts X-rays to a resolution of about 3.8 angstroms (form T2); a crystal of PhFcεRIα₁₋₁₇₆ that belongs to monoclinic space group C2, has cell dimensions of 136.90 angstroms×73.79 angstroms×79.40 angstroms, alpha=gamma=90.0 degrees, beta=117.74 degrees, and diffracts X-rays to a resolution of about 3.2 angstroms (form M2); and a crystal of PhFcεRIα₁₋₁₇₂ that belongs to hexagonal space group P6₁22, has cell dimensions of 58.62 angstroms×58.62 angstroms×229.19 angstroms, alpha=gamma=90.0 degrees, beta=120 degrees, and diffracts X-rays to a resolution of about 3.2 angstroms (form H1)

The present invention includes a 3-D model of an extracellular domain of a FcεRIα protein that substantially represents the atomic coordinates specified in Table 1, Table 5, Table 6, Table 7 or Table 8. The present invention also includes 3-D models that comprise modifications of the model substantially represented by the atomic coordinates specified in Table 1, Table 5, Table 6, Table 7 or Table 8. Each such modification represents a protein that binds to a Fc domain of an antibody. A 3-D model of an extracellular domain of a FcεRIα protein is a representation, or image, that predicts the actual structure of the corresponding protein. As such, a 3-D model is a tool that can be used to probe the relationship between the protein's structure and function at the atomic level and to design muteins (i.e., genetically and/or chemically altered FcRs) having an improved function, such as, but not limited to: increased (i.e., enhanced) stability; increased antibody binding activity, for example, by, increasing the affinity for an antibody by, for example, increasing the association rate and/or decreasing the dissociation rate between a FcR and an antibody or by altering substrate specificity (e.g., enhancing the ability of a FcR of a certain species and class to bind to antibody from another species and/or another antibody class); and/or increased solubility (e.g., reduced aggregation). It is well known to those skilled in the art, however, that a 3-D model of a protein derived by analysis of protein crystals is not identical to the inherent structure of the protein. See, for example, Branden et al., Introduction to Protein Structure, Garland Publishing Inc., New York and London, 1991, especially on page 277, which states “not surprisingly the model never corresponds precisely to the actual crystal.” Furthermore, the model can be subjected to further refinements to more closely correspond to the actual structure of a FcR. Such a refined model, which is an example of a modification of the present invention, is a better predictor of the actual structure and mechanism of action of the protein that the model represents. A refinement of a 3-D model of the present invention refers to an improved model of a FcεRIα protein that can be obtained in a variety of ways known to those skilled in the art. Refinements can include models determined to more preferred degrees of resolution, preferably to about 3.5 angstroms, more preferably to about 3 angstroms, more preferably to about 2.5 angstroms, more preferably to about 2 angstroms, more preferably to about 1.5 angstroms, and even more preferably to about 1 angstrom. Preferred refinements are obtained using the 3-D model as a basis for such improvements.

One embodiment of the present invention is a 3-D model of an extracellular domain of a FcεRIα protein that substantially represents the atomic coordinates specified (i.e., listed) in Table 1.

TABLE 1
Atomic coordinates of PhFcεRIα₁₋₁₇₆, Form M1

| ATOM | ATOM | | | | | | | |
|---|---|---|---|---|---|---|---|---|
| NUMBER | TYPE | RESIDUE | # | X | Y | Z | OCC | B |
| 1 | CB | LYS | 4 | 23.345 | 19.877 | 27.253 | 1.00 | 114.16 |
| 2 | CG | LYS | 4 | 23.455 | 20.034 | 25.744 | 1.00 | 114.16 |
| 3 | CD | LYS | 4 | 23.900 | 21.444 | 25.387 | 1.00 | 114.16 |
| 4 | CE | LYS | 4 | 24.017 | 21.633 | 23.885 | 1.00 | 114.16 |
| 5 | NZ | LYS | 4 | 24.406 | 23.028 | 23.539 | 1.00 | 114.16 |
| 6 | C | LYS | 4 | 23.899 | 17.439 | 27.171 | 1.00 | 98.73 |
| 7 | O | LYS | 4 | 24.999 | 17.777 | 26.726 | 1.00 | 98.73 |
| 8 | N | LYS | 4 | 22.817 | 18.457 | 29.211 | 1.00 | 98.73 |
| 9 | CA | LYS | 4 | 22.920 | 18.482 | 27.721 | 1.00 | 98.73 |
| 10 | N | PRO | 5 | 23.522 | 16.148 | 27.224 | 1.00 | 89.31 |
| 11 | CD | PRO | 5 | 22.385 | 15.565 | 27.963 | 1.00 | 81.52 |
| 12 | CA | PRO | 5 | 24.397 | 15.093 | 26.708 | 1.00 | 89.31 |
| 13 | CB | PRO | 5 | 23.912 | 13.858 | 27.454 | 1.00 | 81.52 |
| 14 | CG | PRO | 5 | 22.445 | 14.102 | 27.562 | 1.00 | 81.52 |
| 15 | C | PRO | 5 | 24.212 | 14.980 | 25.190 | 1.00 | 89.31 |
| 16 | O | PRO | 5 | 23.503 | 15.784 | 24.581 | 1.00 | 89.31 |
| 17 | N | LYS | 6 | 24.844 | 13.992 | 24.575 | 1.00 | 79.33 |
| 18 | CA | LYS | 6 | 24.719 | 13.835 | 23.137 | 1.00 | 79.33 |
| 19 | CB | LYS | 6 | 25.816 | 14.639 | 22.433 | 1.00 | 122.37 |
| 20 | CG | LYS | 6 | 25.411 | 15.180 | 21.073 | 1.00 | 122.37 |
| 21 | CD | LYS | 6 | 26.324 | 16.320 | 20.643 | 1.00 | 122.37 |
| 22 | CE | LYS | 6 | 25.774 | 17.040 | 19.421 | 1.00 | 122.37 |
| 23 | NZ | LYS | 6 | 26.602 | 18.225 | 19.060 | 1.00 | 122.37 |
| 24 | C | LYS | 6 | 24.794 | 12.368 | 22.740 | 1.00 | 79.33 |
| 25 | O | LYS | 6 | 25.644 | 11.622 | 23.231 | 1.00 | 79.33 |
| 26 | N | VAL | 7 | 23.884 | 11.948 | 21.866 | 1.00 | 65.03 |
| 27 | CA | VAL | 7 | 23.879 | 10.567 | 21.409 | 1.00 | 65.03 |
| 28 | CB | VAL | 7 | 22.479 | 10.128 | 20.951 | 1.00 | 74.25 |
| 29 | CG1 | VAL | 7 | 22.530 | 8.711 | 20.408 | 1.00 | 74.25 |
| 30 | CG2 | VAL | 7 | 21.515 | 10.205 | 22.113 | 1.00 | 74.25 |
| 31 | C | VAL | 7 | 24.846 | 10.463 | 20.244 | 1.00 | 65.03 |
| 32 | O | VAL | 7 | 24.829 | 11.290 | 19.328 | 1.00 | 65.03 |
| 33 | N | SER | 8 | 25.713 | 9.462 | 20.299 | 1.00 | 46.54 |
| 34 | CA | SER | 8 | 26.686 | 9.255 | 19.238 | 1.00 | 46.54 |
| 35 | CB | SER | 8 | 28.123 | 9.513 | 19.749 | 1.00 | 64.02 |
| 36 | OG | SER | 8 | 28.482 | 8.670 | 20.836 | 1.00 | 64.02 |
| 37 | C | SER | 8 | 26.517 | 7.815 | 18.780 | 1.00 | 46.54 |
| 38 | O | SER | 8 | 26.109 | 6.955 | 19.567 | 1.00 | 46.54 |
| 39 | N | LEU | 9 | 26.840 | 7.556 | 17.515 | 1.00 | 55.36 |
| 40 | CA | LEU | 9 | 26.674 | 6.227 | 16.945 | 1.00 | 55.36 |
| 41 | CB | LEU | 9 | 25.796 | 6.283 | 15.679 | 1.00 | 45.99 |
| 42 | CG | LEU | 9 | 24.626 | 7.256 | 15.529 | 1.00 | 45.99 |
| 43 | CD1 | LEU | 9 | 23.773 | 6.849 | 14.338 | 1.00 | 45.99 |
| 44 | CD2 | LEU | 9 | 23.784 | 7.246 | 16.761 | 1.00 | 45.99 |
| 45 | C | LEU | 9 | 27.983 | 5.585 | 16.555 | 1.00 | 55.36 |
| 46 | O | LEU | 9 | 28.894 | 6.250 | 16.091 | 1.00 | 55.36 |
| 47 | N | ASN | 10 | 28.060 | 4.274 | 16.713 | 1.00 | 52.82 |
| 48 | CA | ASM | 10 | 29.244 | 3.556 | 16.318 | 1.00 | 52.82 |
| 49 | CB | ASN | 10 | 30.174 | 3.353 | 17.510 | 1.00 | 77.87 |
| 50 | CG | ASN | 10 | 31.366 | 2.495 | 17.166 | 1.00 | 77.87 |
| 51 | OD1 | ASN | 10 | 32.032 | 2.717 | 16.155 | 1.00 | 77.87 |
| 52 | ND2 | ASN | 10 | 31.645 | 1.504 | 18.004 | 1.00 | 77.87 |
| 53 | C | ASN | 10 | 28.816 | 2.215 | 15.753 | 1.00 | 52.82 |
| 54 | O | ASN | 10 | 28.320 | 1.361 | 16.492 | 1.00 | 52.82 |
| 55 | N | PRO | 11 | 28.966 | 2.024 | 14.432 | 1.00 | 54.26 |
| 56 | CD | PRO | 11 | 28.755 | 0.707 | 13.793 | 1.00 | 46.78 |
| 57 | CA | PRO | 11 | 29.503 | 2.975 | 13.454 | 1.00 | 54.26 |
| 58 | CB | PRO | 11 | 29.512 | 2.179 | 12.155 | 1.00 | 46.78 |
| 59 | CG | PRO | 11 | 29.707 | 0.774 | 12.631 | 1.00 | 46.78 |
| 60 | C | PRO | 11 | 28.692 | 4.268 | 13.348 | 1.00 | 54.26 |
| 61 | O | PRO | 11 | 27.541 | 4.332 | 13.775 | 1.00 | 54.26 |
| 62 | N | PRO | 12 | 29.286 | 5.303 | 12.742 | 1.00 | 56.88 |
| 63 | CD | PRO | 12 | 30.615 | 5.189 | 12.120 | 1.00 | 56.23 |
| 64 | CA | PRO | 12 | 28.751 | 6.646 | 12.514 | 1.00 | 56.88 |
| 65 | CB | PRO | 12 | 29.888 | 7.348 | 11.788 | 1.00 | 56.23 |
| 66 | CG | PRO | 12 | 31.093 | 6.592 | 12.191 | 1.00 | 56.23 |
| 67 | C | PRO | 12 | 27.458 | 6.798 | 11.736 | 1.00 | 56.88 |
| 68 | O | PRO | 12 | 26.680 | 7.710 | 12.003 | 1.00 | 56.88 |
| 69 | N | TRP | 13 | 27.255 | 5.930 | 10.751 | 1.00 | 54.29 |
| 70 | CA | TRP | 13 | 26.079 | 5.991 | 9.881 | 1.00 | 54.29 |
| 71 | CB | TRP | 13 | 26.203 | 4.929 | 8.794 | 1.00 | 47.07 |
| 72 | CG | TRP | 13 | 27.629 | 4.697 | 8.423 | 1.00 | 47.07 |
| 73 | CD2 | TRP | 13 | 28.502 | 5.622 | 7.767 | 1.00 | 47.07 |
| 74 | CE2 | TRP | 13 | 29.762 | 4.998 | 7.659 | 1.00 | 47.07 |
| 75 | CE3 | TRP | 13 | 28.341 | 6.919 | 7.266 | 1.00 | 47.07 |
| 76 | CD1 | TRP | 13 | 28.372 | 3.584 | 8.676 | 1.00 | 47.07 |
| 77 | NE1 | TRP | 13 | 29.655 | 3.756 | 8.218 | 1.00 | 47.07 |
| 78 | CZ2 | TRP | 13 | 30.853 | 5.626 | 7.064 | 1.00 | 47.07 |
| 79 | CZ3 | TRP | 13 | 29.419 | 7.536 | 6.679 | 1.00 | 47.07 |
| 80 | CH2 | TRP | 13 | 30.664 | 6.890 | 6.582 | 1.00 | 47.07 |
| 81 | C | TRP | 13 | 24.753 | 5.836 | 10.602 | 1.00 | 54.29 |
| 82 | O | TRP | 13 | 24.571 | 4.912 | 11.389 | 1.00 | 54.29 |
| 83 | N | ASN | 14 | 23.838 | 6.759 | 10.323 | 1.00 | 44.90 |
| 84 | CA | ASN | 14 | 22.513 | 6.758 | 10.925 | 1.00 | 44.90 |
| 85 | CB | ASN | 14 | 22.099 | 8.179 | 11.291 | 1.00 | 62.66 |
| 86 | CG | ASN | 14 | 21.713 | 8.992 | 10.083 | 1.00 | 62.66 |
| 87 | OD1 | ASN | 14 | 22.501 | 9.154 | 9.152 | 1.00 | 62.66 |
| 88 | ND2 | ASN | 14 | 20.489 | 9.505 | 10.085 | 1.00 | 62.66 |
| 89 | C | ASN | 14 | 21.504 | 6.158 | 9.935 | 1.00 | 44.90 |
| 90 | O | ASN | 14 | 20.302 | 6.059 | 10.229 | 1.00 | 44.90 |
| 91 | N | ARG | 15 | 22.006 | 5.777 | 8.759 | 1.00 | 43.26 |
| 92 | CA | ARG | 15 | 21.189 | 5.130 | 7.735 | 1.00 | 43.26 |
| 93 | CB | ARG | 15 | 21.196 | 5.926 | 6.426 | 1.00 | 51.24 |
| 94 | CG | ARG | 15 | 21.031 | 7.419 | 6.623 | 1.00 | 51.24 |
| 95 | CD | ARG | 15 | 21.112 | 8.161 | 5.311 | 1.00 | 51.24 |
| 96 | NE | ARG | 15 | 19.813 | 8.119 | 4.637 | 1.00 | 51.24 |
| 97 | CZ | ARG | 15 | 19.648 | 7.770 | 3.375 | 1.00 | 51.24 |
| 98 | NH1 | ARG | 15 | 20.693 | 7.441 | 2.652 | 1.00 | 51.24 |
| 99 | NH2 | ARG | 15 | 18.442 | 7.743 | 2.849 | 1.00 | 51.24 |
| 100 | C | ARG | 15 | 21.902 | 3.799 | 7.545 | 1.00 | 43.26 |
| 101 | O | ARG | 15 | 23.017 | 3.759 | 7.031 | 1.00 | 43.26 |
| 102 | N | ILE | 16 | 21.258 | 2.719 | 7.981 | 1.00 | 47.47 |
| 103 | CA | ILE | 16 | 21.845 | 1.386 | 7.893 | 1.00 | 47.47 |
| 104 | CB | ILE | 16 | 22.222 | 0.864 | 9.308 | 1.00 | 38.74 |
| 105 | CG2 | ILE | 16 | 23.163 | 1.866 | 10.026 | 1.00 | 38.74 |
| 106 | CG1 | ILE | 16 | 20.944 | 0.673 | 10.139 | 1.00 | 38.74 |
| 107 | CD1 | ILE | 16 | 21.166 | 0.154 | 11.568 | 1.00 | 38.74 |
| 108 | C | ILE | 16 | 20.912 | 0.357 | 7.257 | 1.00 | 47.47 |
| 109 | O | ILE | 16 | 19.711 | 0.579 | 7.111 | 1.00 | 47.47 |
| 110 | N | PHE | 17 | 21.480 | −0.785 | 6.900 | 1.00 | 45.27 |
| 111 | CA | PHE | 17 | 20.721 | −1.874 | 6.309 | 1.00 | 45.27 |
| 112 | CB | PHE | 17 | 21.636 | −2.758 | 5.473 | 1.00 | 33.04 |
| 113 | CG | PHE | 17 | 21.911 | −2.218 | 4.113 | 1.00 | 33.04 |
| 114 | CD1 | PHE | 17 | 23.185 | −2.237 | 3.598 | 1.00 | 33.04 |
| 115 | CD2 | PHE | 17 | 20.864 | −1.725 | 3.322 | 1.00 | 33.04 |
| 116 | CE1 | PHE | 17 | 23.432 | −1.782 | 2.307 | 1.00 | 33.04 |
| 117 | CE2 | PHE | 17 | 21.106 | −1.272 | 2.036 | 1.00 | 33.04 |
| 118 | CZ | PHE | 17 | 22.387 | −1.299 | 1.523 | 1.00 | 33.04 |
| 119 | C | PHE | 17 | 20.026 | −2.748 | 7.334 | 1.00 | 45.27 |
| 120 | O | PHE | 17 | 20.540 | −2.971 | 8.433 | 1.00 | 45.27 |
| 121 | N | LYS | 18 | 18.847 | −3.240 | 6.959 | 1.00 | 48.86 |
| 122 | CA | LYS | 18 | 18.074 | −4.137 | 7.806 | 1.00 | 48.86 |
| 123 | CB | LYS | 18 | 16.848 | −4.630 | 7.035 | 1.00 | 55.91 |
| 124 | CG | LYS | 18 | 16.039 | −5.694 | 7.731 | 1.00 | 55.91 |
| 125 | CD | LYS | 18 | 14.629 | −5.696 | 7.175 | 1.00 | 55.91 |
| 126 | CE | LYS | 18 | 13.744 | −6.718 | 7.865 | 1.00 | 55.91 |
| 127 | NZ | LYS | 18 | 13.936 | −8.075 | 7.298 | 1.00 | 55.91 |
| 128 | C | LYS | 18 | 19.003 | −5.299 | 8.149 | 1.00 | 48.86 |
| 129 | O | LYS | 18 | 19.635 | −5.875 | 7.267 | 1.00 | 48.86 |
| 130 | N | GLY | 19 | 19.106 | −5.627 | 9.429 | 1.00 | 53.46 |
| 131 | CA | GLY | 19 | 19.993 | −6.705 | 9.832 | 1.00 | 53.46 |
| 132 | C | GLY | 19 | 21.381 | −6.284 | 10.311 | 1.00 | 53.46 |
| 133 | O | GLY | 19 | 22.111 | −7.117 | 10.837 | 1.00 | 53.46 |
| 134 | N | GLU | 20 | 21.758 | −5.016 | 10.134 | 1.00 | 46.04 |
| 135 | CA | GLU | 20 | 23.073 | −4.533 | 10.576 | 1.00 | 46.04 |
| 136 | CB | GLU | 20 | 23.553 | −3.372 | 9.700 | 1.00 | 45.53 |
| 137 | CG | GLU | 20 | 23.544 | −3.671 | 8.197 | 1.00 | 45.53 |
| 138 | CD | GLU | 20 | 24.253 | −2.611 | 7.347 | 1.00 | 45.53 |
| 139 | OE1 | GLU | 20 | 24.049 | −1.393 | 7.587 | 1.00 | 45.53 |
| 140 | OE2 | GLU | 20 | 25.008 | −3.004 | 6.423 | 1.00 | 45.53 |
| 141 | C | GLU | 20 | 23.046 | −4.083 | 12.039 | 1.00 | 46.04 |
| 142 | O | GLU | 20 | 21.980 | −3.958 | 12.654 | 1.00 | 46.04 |
| 143 | N | ASN | 21 | 24.223 | −3.845 | 12.607 | 1.00 | 50.99 |
| 144 | CA | ASN | 21 | 24.297 | −3.422 | 13.994 | 1.00 | 50.99 |
| 145 | CB | ASN | 21 | 25.255 | −4.321 | 14.790 | 1.00 | 67.08 |
| 146 | CG | ASN | 21 | 24.817 | −5.776 | 14.825 | 1.00 | 67.08 |
| 147 | OD1 | ASN | 21 | 23.634 | −6.077 | 15.049 | 1.00 | 67.08 |
| 148 | ND2 | ASN | 21 | 25.782 | −6.675 | 14.619 | 1.00 | 67.08 |
| 149 | C | ASN | 21 | 24.765 | −1.985 | 14.118 | 1.00 | 50.99 |
| 150 | O | ASN | 21 | 25.533 | −1.493 | 13.290 | 1.00 | 50.99 |
| 151 | N | VAL | 22 | 24.291 | −1.317 | 15.164 | 1.00 | 48.02 |
| 152 | CA | VAL | 22 | 24.674 | 0.058 | 15.453 | 1.00 | 48.02 |
| 153 | CB | VAL | 22 | 23.752 | 1.086 | 14.742 | 1.00 | 39.09 |
| 154 | CG1 | VAL | 22 | 22.313 | 0.924 | 15.215 | 1.00 | 39.09 |
| 155 | CG2 | VAL | 22 | 24.243 | 2.499 | 15.023 | 1.00 | 39.09 |
| 156 | C | VAL | 22 | 24.552 | 0.241 | 16.964 | 1.00 | 48.02 |
| 157 | O | VAL | 22 | 23.568 | −0.203 | 17.577 | 1.00 | 48.02 |
| 158 | N | THR | 23 | 25.558 | 0.870 | 17.570 | 1.00 | 52.73 |
| 159 | CA | THR | 23 | 25.530 | 1.102 | 19.013 | 1.00 | 52.73 |
| 160 | CB | THR | 23 | 26.848 | 0.666 | 19.686 | 1.00 | 65.60 |
| 161 | OG1 | THR | 23 | 26.999 | −0.754 | 19.570 | 1.00 | 65.60 |
| 162 | CG2 | THR | 23 | 26.849 | 1.059 | 21.162 | 1.00 | 65.60 |
| 163 | C | THR | 23 | 25.313 | 2.577 | 19.294 | 1.00 | 52.73 |
| 164 | O | THR | 23 | 25.946 | 3.422 | 18.673 | 1.00 | 52.73 |
| 165 | N | LEU | 24 | 24.407 | 2.883 | 20.214 | 1.00 | 47.77 |
| 166 | CA | LEU | 24 | 24.136 | 4.267 | 20.576 | 1.00 | 47.77 |
| 167 | CB | LEU | 24 | 22.632 | 4.561 | 20.543 | 1.00 | 58.94 |
| 168 | CG | LEU | 24 | 21.813 | 4.200 | 19.303 | 1.00 | 58.94 |
| 169 | CD1 | LEU | 24 | 20.429 | 4.824 | 19.406 | 1.00 | 58.94 |
| 170 | CD2 | LEU | 24 | 22.509 | 4.704 | 18.068 | 1.00 | 58.94 |
| 171 | C | LEU | 24 | 24.673 | 4.528 | 21.980 | 1.00 | 47.77 |
| 172 | O | LEU | 24 | 24.287 | 3.858 | 22.950 | 1.00 | 47.77 |
| 173 | N | THR | 25 | 25.563 | 5.510 | 22.085 | 1.00 | 57.92 |
| 174 | CA | THR | 25 | 26.155 | 5.867 | 23.371 | 1.00 | 57.92 |
| 175 | CB | THR | 25 | 27.700 | 5.784 | 23.316 | 1.00 | 61.52 |
| 176 | OG1 | THR | 25 | 28.091 | 4.446 | 22.995 | 1.00 | 61.52 |
| 177 | CG2 | THR | 25 | 28.292 | 6.164 | 24.669 | 1.00 | 61.52 |
| 178 | C | THR | 25 | 25.738 | 7.275 | 23.818 | 1.00 | 57.92 |
| 179 | O | THR | 25 | 25.732 | 8.211 | 23.018 | 1.00 | 57.92 |
| 180 | N | CYS | 26 | 25.397 | 7.394 | 25.101 | 1.00 | 87.83 |
| 181 | CA | CYS | 26 | 24.998 | 8.662 | 25.685 | 1.00 | 87.83 |
| 182 | C | CYS | 26 | 26.319 | 9.363 | 26.031 | 1.00 | 87.83 |
| 183 | O | CYS | 26 | 27.330 | 8.698 | 26.256 | 1.00 | 87.83 |
| 184 | CB | CYS | 26 | 24.149 | 8.404 | 26.926 | 1.00 | 68.33 |
| 185 | SG | CYS | 26 | 23.114 | 9.775 | 27.559 | 1.00 | 68.33 |
| 186 | N | ASN | 27 | 26.315 | 10.692 | 26.067 | 1.00 | 98.51 |
| 187 | CA | ASN | 27 | 27.538 | 11.474 | 26.307 | 1.00 | 98.51 |
| 188 | CB | ASN | 27 | 27.183 | 12.865 | 26.803 | 1.00 | 123.41 |
| 189 | CG | ASN | 27 | 27.922 | 13.945 | 26.038 | 1.00 | 123.41 |
| 190 | OD1 | ASN | 27 | 29.067 | 13.760 | 25.618 | 1.00 | 123.41 |
| 191 | ND2 | ASN | 27 | 27.268 | 15.085 | 25.851 | 1.00 | 123.41 |
| 192 | C | ASN | 27 | 28.671 | 10.911 | 27.174 | 1.00 | 98.51 |
| 193 | O | ASN | 27 | 28.481 | 9.968 | 27.937 | 1.00 | 98.51 |
| 194 | N | GLY | 28 | 29.849 | 11.531 | 27.053 | 1.00 | 112.08 |
| 195 | CA | GLY | 28 | 31.050 | 11.111 | 27.774 | 1.00 | 112.08 |
| 196 | C | GLY | 28 | 31.283 | 11.487 | 29.235 | 1.00 | 112.08 |
| 197 | O | GLY | 28 | 32.175 | 10.929 | 29.874 | 1.00 | 112.08 |
| 198 | N | ASN | 29 | 30.513 | 12.430 | 29.772 | 1.00 | 117.67 |
| 199 | CA | ASN | 29 | 30.674 | 12.818 | 31.175 | 1.00 | 117.67 |
| 200 | CB | ASN | 29 | 30.018 | 14.176 | 31.464 | 1.00 | 132.23 |
| 201 | CG | ASN | 29 | 30.579 | 15.301 | 30.619 | 1.00 | 132.23 |
| 202 | OD1 | ASN | 29 | 31.792 | 15.505 | 30.559 | 1.00 | 132.23 |
| 203 | ND2 | ASN | 29 | 29.693 | 16.049 | 29.971 | 1.00 | 132.23 |
| 204 | C | ASN | 29 | 30.009 | 11.779 | 32.077 | 1.00 | 117.67 |
| 205 | O | ASN | 29 | 30.259 | 11.737 | 33.277 | 1.00 | 117.67 |
| 206 | N | ASN | 30 | 29.158 | 10.943 | 31.489 | 1.00 | 110.72 |
| 207 | CA | ASN | 30 | 28.423 | 9.921 | 32.235 | 1.00 | 110.72 |
| 208 | CB | ASN | 30 | 27.236 | 9.430 | 31.395 | 1.00 | 135.09 |
| 209 | CG | ASN | 30 | 26.331 | 8.468 | 32.153 | 1.00 | 135.09 |
| 210 | OD1 | ASN | 30 | 26.684 | 7.989 | 33.231 | 1.00 | 135.09 |
| 211 | ND2 | ASN | 30 | 25.163 | 8.183 | 31.590 | 1.00 | 135.09 |
| 212 | C | ASN | 30 | 29.267 | 8.721 | 32.680 | 1.00 | 110.72 |
| 213 | O | ASN | 30 | 29.834 | 8.000 | 31.853 | 1.00 | 110.72 |
| 214 | N | PHE | 31 | 29.338 | 8.509 | 33.995 | 1.00 | 129.04 |
| 215 | CA | PHE | 31 | 30.095 | 7.397 | 34.559 | 1.00 | 129.04 |
| 216 | CB | PHE | 31 | 31.178 | 7.900 | 35.519 | 1.00 | 95.73 |
| 217 | CG | PHE | 31 | 32.321 | 8.589 | 34.837 | 1.00 | 95.73 |
| 218 | CD1 | PHE | 31 | 32.106 | 9.716 | 34.059 | 1.00 | 95.73 |
| 219 | CD2 | PHE | 31 | 33.619 | 8.127 | 34.993 | 1.00 | 95.73 |
| 220 | CE1 | PHE | 31 | 33.166 | 10.380 | 33.440 | 1.00 | 95.73 |
| 221 | CE2 | PHE | 31 | 34.687 | 8.784 | 34.378 | 1.00 | 95.73 |
| 222 | CZ | PHE | 31 | 34.458 | 9.915 | 33.603 | 1.00 | 95.73 |
| 223 | C | PHE | 31 | 29.181 | 6.440 | 35.308 | 1.00 | 129.04 |
| 224 | O | PHE | 31 | 28.188 | 6.850 | 35.908 | 1.00 | 129.04 |
| 225 | N | PHE | 32 | 29.531 | 5.160 | 35.260 | 1.00 | 141.76 |
| 226 | CA | PHE | 32 | 28.775 | 4.109 | 35.937 | 1.00 | 141.76 |
| 227 | CB | PHE | 32 | 29.529 | 2.765 | 35.876 | 1.00 | 141.76 |
| 228 | CG | PHE | 32 | 30.787 | 2.788 | 35.031 | 1.00 | 141.76 |
| 229 | CD1 | PHE | 32 | 31.814 | 3.706 | 35.280 | 1.00 | 141.76 |
| 230 | CD2 | PHE | 32 | 30.953 | 1.870 | 33.993 | 1.00 | 141.76 |
| 231 | CE1 | PHE | 32 | 32.985 | 3.712 | 34.501 | 1.00 | 141.76 |
| 232 | CE2 | PHE | 32 | 32.118 | 1.867 | 33.209 | 1.00 | 141.76 |
| 233 | CZ | PHE | 32 | 33.134 | 2.787 | 33.464 | 1.00 | 141.76 |
| 234 | C | PHE | 32 | 28.562 | 4.481 | 37.408 | 1.00 | 141.76 |
| 235 | O | PHE | 32 | 29.410 | 5.140 | 38.017 | 1.00 | 141.76 |
| 236 | N | GLU | 33 | 27.433 | 4.055 | 37.969 | 1.00 | 141.76 |
| 237 | CA | GLU | 33 | 27.103 | 4.330 | 39.369 | 1.00 | 141.76 |
| 238 | CB | GLU | 33 | 28.229 | 3.831 | 40.292 | 1.00 | 141.76 |
| 239 | CG | GLU | 33 | 28.491 | 2.332 | 40.223 | 1.00 | 141.76 |
| 240 | CD | GLU | 33 | 27.251 | 1.496 | 40.515 | 1.00 | 141.76 |
| 241 | OE1 | GLU | 33 | 26.216 | 2.073 | 40.920 | 1.00 | 141.76 |
| 242 | OE2 | GLU | 33 | 27.313 | 0.256 | 40.344 | 1.00 | 141.76 |
| 243 | C | GLU | 33 | 26.784 | 5.802 | 39.682 | 1.00 | 141.76 |
| 244 | O | GLU | 33 | 26.382 | 6.125 | 40.803 | 1.00 | 141.76 |
| 245 | N | VAL | 34 | 26.967 | 6.689 | 38.705 | 1.00 | 137.33 |
| 246 | CA | VAL | 34 | 26.663 | 8.105 | 38.896 | 1.00 | 137.33 |
| 247 | CB | VAL | 34 | 27.428 | 8.997 | 37.878 | 1.00 | 109.45 |
| 248 | CG1 | VAL | 34 | 26.940 | 10.441 | 37.974 | 1.00 | 109.45 |
| 249 | CG2 | VAL | 34 | 28.929 | 8.922 | 38.140 | 1.00 | 109.45 |
| 250 | C | VAL | 34 | 25.167 | 8.237 | 38.637 | 1.00 | 137.33 |
| 251 | O | VAL | 34 | 24.368 | 8.503 | 39.545 | 1.00 | 137.33 |
| 252 | N | SER | 35 | 24.807 | 8.021 | 37.375 | 1.00 | 141.76 |
| 253 | CA | SER | 35 | 23.432 | 8.110 | 36.909 | 1.00 | 141.76 |
| 254 | CB | SER | 35 | 23.189 | 9.522 | 36.351 | 1.00 | 133.69 |
| 255 | OG | SER | 35 | 21.955 | 9.652 | 35.667 | 1.00 | 133.69 |
| 256 | C | SER | 35 | 23.164 | 7.055 | 35.828 | 1.00 | 141.76 |
| 257 | O | SER | 35 | 23.825 | 7.036 | 34.785 | 1.00 | 141.76 |
| 258 | N | SER | 36 | 22.226 | 6.148 | 36.087 | 1.00 | 88.62 |
| 259 | CA | SER | 36 | 21.891 | 5.151 | 35.080 | 1.00 | 88.62 |
| 260 | CB | SER | 36 | 20.818 | 4.198 | 35.591 | 1.00 | 92.65 |
| 261 | OG | SER | 36 | 19.623 | 4.905 | 35.850 | 1.00 | 92.65 |
| 262 | C | SER | 36 | 21.335 | 6.012 | 33.959 | 1.00 | 88.62 |
| 263 | O | SER | 36 | 20.928 | 7.150 | 34.193 | 1.00 | 88.62 |
| 264 | N | THR | 37 | 21.332 | 5.495 | 32.741 | 1.00 | 66.50 |
| 265 | CA | THR | 37 | 20.833 | 6.279 | 31.625 | 1.00 | 66.50 |
| 266 | CB | THR | 37 | 21.718 | 6.049 | 30.395 | 1.00 | 64.10 |
| 267 | OG1 | THR | 37 | 23.085 | 6.313 | 30.748 | 1.00 | 64.10 |
| 268 | CG2 | THR | 37 | 21.299 | 6.969 | 29.254 | 1.00 | 64.10 |
| 269 | C | THR | 37 | 19.369 | 6.003 | 31.268 | 1.00 | 66.50 |
| 270 | O | THR | 37 | 18.855 | 4.912 | 31.478 | 1.00 | 66.50 |
| 271 | N | LYS | 38 | 18.693 | 7.025 | 30.764 | 1.00 | 59.63 |
| 272 | CA | LYS | 38 | 17.304 | 6.899 | 30.331 | 1.00 | 59.63 |
| 273 | CB | LYS | 38 | 16.430 | 7.945 | 31.017 | 1.00 | 48.08 |
| 274 | CG | LYS | 38 | 15.696 | 7.418 | 32.220 | 1.00 | 48.08 |
| 275 | CD | LYS | 38 | 15.075 | 8.535 | 33.017 | 1.00 | 48.08 |
| 276 | CE | LYS | 38 | 14.471 | 7.971 | 34.289 | 1.00 | 48.08 |
| 277 | NZ | LYS | 38 | 13.893 | 9.058 | 35.108 | 1.00 | 48.08 |
| 278 | C | LYS | 38 | 17.274 | 7.120 | 28.820 | 1.00 | 59.63 |
| 279 | O | LYS | 38 | 17.770 | 8.140 | 28.343 | 1.00 | 59.63 |
| 280 | N | TRP | 39 | 16.719 | 6.166 | 28.068 | 1.00 | 49.53 |
| 281 | CA | TRP | 39 | 16.634 | 6.286 | 26.599 | 1.00 | 49.53 |
| 282 | CB | TRP | 39 | 17.250 | 5.060 | 25.919 | 1.00 | 52.24 |
| 283 | CG | TRP | 39 | 18.742 | 4.956 | 26.016 | 1.00 | 52.24 |
| 284 | CD2 | TRP | 39 | 19.701 | 5.542 | 25.124 | 1.00 | 52.24 |
| 285 | CE2 | TRP | 39 | 20.985 | 5.189 | 25.598 | 1.00 | 52.24 |
| 286 | CE3 | TRP | 39 | 19.601 | 6.338 | 23.972 | 1.00 | 52.24 |
| 287 | CD1 | TRP | 39 | 19.461 | 4.288 | 26.966 | 1.00 | 52.24 |
| 288 | NE1 | TRP | 39 | 20.809 | 4.422 | 26.720 | 1.00 | 52.24 |
| 289 | CZ2 | TRP | 39 | 22.158 | 5.601 | 24.961 | 1.00 | 52.24 |
| 290 | CZ3 | TRP | 39 | 20.772 | 6.750 | 23.339 | 1.00 | 52.24 |
| 291 | CH2 | TRP | 39 | 22.032 | 6.380 | 23.837 | 1.00 | 52.24 |
| 292 | C | TRP | 39 | 15.194 | 6.450 | 26.090 | 1.00 | 49.53 |
| 293 | O | TRP | 39 | 14.270 | 5.831 | 26.608 | 1.00 | 49.53 |
| 294 | N | PHE | 40 | 15.000 | 7.283 | 25.079 | 1.00 | 52.82 |
| 295 | CA | PHE | 40 | 13.662 | 7.470 | 24.529 | 1.00 | 52.82 |
| 296 | CB | PHE | 40 | 13.159 | 8.894 | 24.792 | 1.00 | 55.71 |
| 297 | CG | PHE | 40 | 13.062 | 9.229 | 26.255 | 1.00 | 55.71 |
| 298 | CD1 | PHE | 40 | 14.209 | 9.497 | 27.000 | 1.00 | 55.71 |
| 299 | CD2 | PHE | 40 | 11.833 | 9.208 | 26.909 | 1.00 | 55.71 |
| 300 | CE1 | PHE | 40 | 14.140 | 9.719 | 28.368 | 1.00 | 55.71 |
| 301 | CE2 | PHE | 40 | 11.755 | 9.437 | 28.287 | 1.00 | 55.71 |
| 302 | CZ | PHE | 40 | 12.916 | 9.691 | 29.012 | 1.00 | 55.71 |
| 303 | C | PHE | 40 | 13.637 | 7.159 | 23.029 | 1.00 | 52.82 |
| 304 | O | PHE | 40 | 14.294 | 7.823 | 22.228 | 1.00 | 52.82 |
| 305 | N | HIS | 41 | 12.896 | 6.117 | 22.665 | 1.00 | 42.70 |
| 306 | CA | HIS | 41 | 12.766 | 5.703 | 21.269 | 1.00 | 42.70 |
| 307 | CB | HIS | 41 | 12.801 | 4.176 | 21.186 | 1.00 | 48.50 |
| 308 | CG | HIS | 41 | 12.708 | 3.643 | 19.795 | 1.00 | 48.50 |
| 309 | CD2 | HIS | 41 | 12.249 | 2.462 | 19.318 | 1.00 | 48.50 |
| 310 | ND1 | HIS | 41 | 13.128 | 4.360 | 18.698 | 1.00 | 48.50 |
| 311 | CE1 | HIS | 41 | 12.931 | 3.647 | 17.604 | 1.00 | 48.50 |
| 312 | NE2 | HIS | 41 | 12.397 | 2.490 | 17.954 | 1.00 | 48.50 |
| 313 | C | HIS | 41 | 11.408 | 6.261 | 20.842 | 1.00 | 42.70 |
| 314 | O | HIS | 41 | 10.387 | 5.917 | 21.434 | 1.00 | 42.70 |
| 315 | N | ASN | 42 | 11.419 | 7.145 | 19.845 | 1.00 | 49.72 |
| 316 | CA | ASN | 42 | 10.184 | 7.785 | 19.375 | 1.00 | 49.72 |
| 317 | CB | ASN | 42 | 9.253 | 6.782 | 18.668 | 1.00 | 43.66 |
| 318 | CG | ASN | 42 | 9.743 | 6.393 | 17.280 | 1.00 | 43.66 |
| 319 | OD1 | ASN | 42 | 10.632 | 7.053 | 16.729 | 1.00 | 43.66 |
| 320 | ND2 | ASN | 42 | 9.156 | 5.340 | 16.708 | 1.00 | 43.66 |
| 321 | C | ASN | 42 | 9.447 | 8.391 | 20.562 | 1.00 | 49.72 |
| 322 | O | ASN | 42 | 8.220 | 8.372 | 20.609 | 1.00 | 49.72 |
| 323 | N | GLY | 43 | 10.202 | 8.901 | 21.533 | 1.00 | 60.04 |
| 324 | CA | GLY | 43 | 9.588 | 9.505 | 22.706 | 1.00 | 60.04 |
| 325 | C | GLY | 43 | 9.100 | 8.539 | 23.778 | 1.00 | 60.04 |
| 326 | O | GLY | 43 | 8.465 | 8.961 | 24.748 | 1.00 | 60.04 |
| 327 | N | SER | 44 | 9.377 | 7.247 | 23.615 | 1.00 | 55.75 |
| 328 | CA | SER | 44 | 8.948 | 6.249 | 24.592 | 1.00 | 55.75 |
| 329 | CB | SER | 44 | 8.280 | 5.058 | 23.908 | 1.00 | 71.51 |
| 330 | OG | SER | 44 | 6.988 | 5.397 | 23.456 | 1.00 | 71.51 |
| 331 | C | SER | 44 | 10.118 | 5.744 | 25.405 | 1.00 | 55.75 |
| 332 | O | SER | 44 | 11.122 | 5.289 | 24.855 | 1.00 | 55.75 |
| 333 | N | LEU | 45 | 9.981 | 5.821 | 26.723 | 1.00 | 58.69 |
| 334 | CA | LEU | 45 | 11.040 | 5.380 | 27.609 | 1.00 | 58.69 |
| 335 | CB | LEU | 45 | 10.639 | 5.585 | 29.068 | 1.00 | 58.46 |
| 336 | CG | LEU | 45 | 11.647 | 5.140 | 30.129 | 1.00 | 58.46 |
| 337 | CD1 | LEU | 45 | 12.963 | 5.906 | 30.005 | 1.00 | 58.46 |
| 338 | CD2 | LEU | 45 | 11.017 | 5.358 | 31.491 | 1.00 | 58.46 |
| 339 | C | LEU | 45 | 11.375 | 3.925 | 27.366 | 1.00 | 58.69 |
| 340 | O | LEU | 45 | 10.508 | 3.054 | 27.398 | 1.00 | 58.69 |
| 341 | N | SER | 46 | 12.650 | 3.677 | 27.116 | 1.00 | 59.18 |
| 342 | CA | SER | 46 | 13.138 | 2.336 | 26.864 | 1.00 | 59.18 |
| 343 | CB | SER | 46 | 14.437 | 2.402 | 26.077 | 1.00 | 54.40 |
| 344 | OG | SER | 46 | 15.025 | 1.120 | 26.000 | 1.00 | 54.40 |
| 345 | C | SER | 46 | 13.388 | 1.591 | 28.165 | 1.00 | 59.18 |
| 346 | O | SER | 46 | 13.461 | 2.192 | 29.236 | 1.00 | 59.18 |
| 347 | N | GLU | 47 | 13.507 | 0.274 | 28.073 | 1.00 | 69.21 |
| 348 | CA | GLU | 47 | 13.785 | −0.524 | 29.252 | 1.00 | 69.21 |
| 349 | CB | GLU | 47 | 13.256 | −1.944 | 29.080 | 1.00 | 88.33 |
| 350 | CG | GLU | 47 | 11.752 | −2.050 | 29.190 | 1.00 | 88.33 |
| 351 | CD | GLU | 47 | 11.284 | −3.483 | 29.278 | 1.00 | 88.33 |
| 352 | OE1 | GLU | 47 | 11.349 | −4.198 | 28.256 | 1.00 | 88.33 |
| 353 | OE2 | GLU | 47 | 10.860 | −3.898 | 30.377 | 1.00 | 88.33 |
| 354 | C | GLU | 47 | 15.297 | −0.541 | 29.433 | 1.00 | 69.21 |
| 355 | O | GLU | 47 | 15.807 | −0.973 | 30.462 | 1.00 | 69.21 |
| 356 | N | GLU | 48 | 16.003 | −0.057 | 28.415 | 1.00 | 63.82 |
| 357 | CA | GLU | 48 | 17.456 | 0.011 | 28.433 | 1.00 | 63.82 |
| 358 | CB | GLU | 48 | 17.980 | 0.306 | 27.025 | 1.00 | 75.50 |
| 359 | CG | GLU | 48 | 19.483 | 0.478 | 26.950 | 1.00 | 75.50 |
| 360 | CD | GLU | 48 | 20.223 | −0.738 | 27.466 | 1.00 | 75.50 |
| 361 | OE1 | GLU | 48 | 20.152 | −1.802 | 26.810 | 1.00 | 75.50 |
| 362 | OE2 | GLU | 48 | 20.863 | −0.628 | 28.534 | 1.00 | 75.50 |
| 363 | C | GLU | 48 | 17.902 | 1.113 | 29.394 | 1.00 | 63.82 |
| 364 | O | GLU | 48 | 17.454 | 2.255 | 29.284 | 1.00 | 63.82 |
| 365 | N | THR | 49 | 18.792 | 0.772 | 30.322 | 1.00 | 72.39 |
| 366 | CA | THR | 49 | 19.275 | 1.737 | 31.303 | 1.00 | 72.39 |
| 367 | CB | THR | 49 | 18.867 | 1.316 | 32.727 | 1.00 | 60.69 |
| 368 | OG1 | THR | 49 | 19.140 | −0.079 | 32.907 | 1.00 | 60.69 |
| 369 | CG2 | THR | 49 | 17.381 | 1.573 | 32.953 | 1.00 | 60.69 |
| 370 | C | THR | 49 | 20.780 | 2.007 | 31.294 | 1.00 | 72.39 |
| 371 | O | THR | 49 | 21.247 | 2.896 | 31.998 | 1.00 | 72.39 |
| 372 | N | ASN | 50 | 21.543 | 1.251 | 30.509 | 1.00 | 68.99 |
| 373 | CA | ASN | 50 | 22.991 | 1.473 | 30.445 | 1.00 | 68.99 |
| 374 | CB | ASN | 50 | 23.710 | 0.247 | 29.879 | 1.00 | 96.77 |
| 375 | CG | ASN | 50 | 23.508 | −0.989 | 30.733 | 1.00 | 96.77 |
| 376 | OD1 | ASN | 50 | 23.625 | −0.933 | 31.956 | 1.00 | 96.77 |
| 377 | ND2 | ASN | 50 | 23.209 | −2.114 | 30.092 | 1.00 | 96.77 |
| 378 | C | ASN | 50 | 23.294 | 2.693 | 29.579 | 1.00 | 68.99 |
| 379 | O | ASN | 50 | 22.424 | 3.178 | 28.856 | 1.00 | 68.99 |
| 380 | N | SER | 51 | 24.527 | 3.186 | 29.655 | 1.00 | 51.99 |
| 381 | CA | SER | 51 | 24.927 | 4.369 | 28.892 | 1.00 | 51.99 |
| 382 | CB | SER | 51 | 26.304 | 4.843 | 29.349 | 1.00 | 64.53 |
| 383 | OG | SER | 51 | 27.281 | 3.878 | 28.998 | 1.00 | 64.53 |
| 384 | C | SER | 51 | 24.978 | 4.074 | 27.391 | 1.00 | 51.99 |
| 385 | O | SER | 51 | 25.024 | 4.987 | 26.569 | 1.00 | 51.99 |
| 386 | N | SER | 52 | 24.998 | 2.793 | 27.045 | 1.00 | 68.17 |
| 387 | CA | SER | 52 | 25.035 | 2.395 | 25.650 | 1.00 | 68.17 |
| 388 | CB | SER | 52 | 26.351 | 1.690 | 25.346 | 1.00 | 64.58 |
| 389 | OG | SER | 52 | 27.361 | 2.655 | 25.123 | 1.00 | 64.58 |
| 390 | C | SER | 52 | 23.866 | 1.502 | 25.248 | 1.00 | 68.17 |
| 391 | O | SER | 52 | 23.621 | 0.468 | 25.864 | 1.00 | 68.17 |
| 392 | N | LEU | 53 | 23.136 | 1.927 | 24.221 | 1.00 | 42.59 |
| 393 | CA | LEU | 53 | 22.001 | 1.160 | 23.704 | 1.00 | 42.59 |
| 394 | CB | LEU | 53 | 20.856 | 2.108 | 23.328 | 1.00 | 56.84 |
| 395 | CG | LEU | 53 | 19.581 | 1.569 | 22.678 | 1.00 | 56.84 |
| 396 | CD1 | LEU | 53 | 19.134 | 0.285 | 23.349 | 1.00 | 56.84 |
| 397 | CD2 | LEU | 53 | 18.501 | 2.627 | 22.801 | 1.00 | 56.84 |
| 398 | C | LEU | 53 | 22.494 | 0.407 | 22.473 | 1.00 | 42.59 |
| 399 | O | LEU | 53 | 23.049 | 1.009 | 21.545 | 1.00 | 42.59 |
| 400 | N | ASN | 54 | 22.330 | −0.911 | 22.479 | 1.00 | 55.75 |
| 401 | CA | ASN | 54 | 22.762 | −1.722 | 21.349 | 1.00 | 55.75 |
| 402 | CB | ASN | 54 | 23.533 | −2.950 | 21.820 | 1.00 | 73.05 |
| 403 | CG | ASN | 54 | 24.921 | −2.613 | 22.300 | 1.00 | 73.05 |
| 404 | OD1 | ASN | 54 | 25.717 | −2.024 | 21.565 | 1.00 | 73.05 |
| 405 | ND2 | ASN | 54 | 25.225 | −2.984 | 23.539 | 1.00 | 73.05 |
| 406 | C | ASN | 54 | 21.592 | −2.177 | 20.493 | 1.00 | 55.75 |
| 407 | O | ASN | 54 | 20.643 | −2.806 | 20.979 | 1.00 | 55.75 |
| 408 | N | ILE | 55 | 21.660 | −1.835 | 19.212 | 1.00 | 67.23 |
| 409 | CA | ILE | 55 | 20.623 | −2.237 | 18.281 | 1.00 | 67.23 |
| 410 | CB | ILE | 55 | 20.258 | −1.098 | 17.338 | 1.00 | 49.71 |
| 411 | CG2 | ILE | 55 | 19.390 | −1.625 | 16.199 | 1.00 | 49.71 |
| 412 | CG1 | ILE | 55 | 19.512 | −0.026 | 18.124 | 1.00 | 49.71 |
| 413 | CD1 | ILE | 55 | 19.172 | 1.189 | 17.333 | 1.00 | 49.71 |
| 414 | C | ILE | 55 | 21.209 | −3.398 | 17.498 | 1.00 | 67.23 |
| 415 | O | ILE | 55 | 22.197 | −3.231 | 16.776 | 1.00 | 67.23 |
| 416 | N | VAL | 56 | 20.618 | −4.576 | 17.674 | 1.00 | 59.54 |
| 417 | CA | VAL | 56 | 21.101 | −5.774 | 16.993 | 1.00 | 59.54 |
| 418 | CB | VAL | 56 | 21.340 | −6.907 | 18.013 | 1.00 | 69.51 |
| 419 | CG1 | VAL | 56 | 21.949 | −8.114 | 17.311 | 1.00 | 69.51 |
| 420 | CG2 | VAL | 56 | 22.262 | −6.419 | 19.125 | 1.00 | 69.51 |
| 421 | C | VAL | 56 | 20.152 | −6.270 | 15.898 | 1.00 | 59.54 |
| 422 | O | VAL | 56 | 18.932 | −6.289 | 16.086 | 1.00 | 59.54 |
| 423 | N | ASN | 57 | 20.716 | −6.684 | 14.763 | 1.00 | 58.29 |
| 424 | CA | ASN | 57 | 19.932 | −7.171 | 13.618 | 1.00 | 58.29 |
| 425 | CB | ASN | 57 | 19.399 | −8.588 | 13.869 | 1.00 | 80.36 |
| 426 | CG | ASN | 57 | 20.503 | −9.631 | 13.901 | 1.00 | 80.36 |
| 427 | OD1 | ASN | 57 | 21.319 | −9.723 | 12.977 | 1.00 | 80.36 |
| 428 | ND2 | ASN | 57 | 20.534 | −10.428 | 14.966 | 1.00 | 80.36 |
| 429 | C | ASN | 57 | 18.788 | −6.195 | 13.433 | 1.00 | 58.29 |
| 430 | O | ASN | 57 | 17.619 | −6.517 | 13.660 | 1.00 | 58.29 |
| 431 | N | ALA | 58 | 19.166 | −4.991 | 13.027 | 1.00 | 46.57 |
| 432 | CA | ALA | 58 | 18.252 | −3.885 | 12.847 | 1.00 | 46.57 |
| 433 | CB | ALA | 58 | 18.982 | −2.774 | 12.075 | 1.00 | 27.34 |
| 434 | C | ALA | 58 | 16.915 | −4.227 | 12.170 | 1.00 | 46.57 |
| 435 | O | ALA | 58 | 16.880 | −4.920 | 11.141 | 1.00 | 46.57 |
| 436 | N | LYS | 59 | 15.823 | −3.732 | 12.737 | 1.00 | 53.79 |
| 437 | CA | LYS | 59 | 14.520 | −3.949 | 12.146 | 1.00 | 53.79 |
| 438 | CB | LYS | 59 | 13.543 | −4.514 | 13.169 | 1.00 | 70.29 |
| 439 | CG | LYS | 59 | 14.035 | −5.733 | 13.911 | 1.00 | 70.29 |
| 440 | CD | LYS | 59 | 13.157 | −5.989 | 15.115 | 1.00 | 70.29 |
| 441 | CE | LYS | 59 | 13.770 | −7.023 | 16.036 | 1.00 | 70.29 |
| 442 | NZ | LYS | 59 | 12.856 | −7.266 | 17.181 | 1.00 | 70.29 |
| 443 | C | LYS | 59 | 14.033 | −2.579 | 11.682 | 1.00 | 53.79 |
| 444 | O | LYS | 59 | 14.593 | −1.549 | 12.062 | 1.00 | 53.79 |
| 445 | N | PHE | 60 | 13.011 | −2.563 | 10.839 | 1.00 | 57.14 |
| 446 | CA | PHE | 60 | 12.473 | −1.301 | 10.359 | 1.00 | 57.14 |
| 447 | CB | PHE | 60 | 11.350 | −1.549 | 9.355 | 1.00 | 75.29 |
| 448 | CG | PHE | 60 | 11.823 | −2.022 | 8.019 | 1.00 | 75.29 |
| 449 | CD1 | PHE | 60 | 11.028 | −2.876 | 7.259 | 1.00 | 75.29 |
| 450 | CD2 | PHE | 60 | 13.043 | −1.596 | 7.499 | 1.00 | 75.29 |
| 451 | CE1 | PHE | 60 | 11.437 | −3.304 | 5.999 | 1.00 | 75.29 |
| 452 | CE2 | PHE | 60 | 13.465 | −2.016 | 6.237 | 1.00 | 75.29 |
| 453 | CZ | PHE | 60 | 12.657 | −2.873 | 5.485 | 1.00 | 75.29 |
| 454 | C | PHE | 60 | 11.922 | −0.568 | 11.567 | 1.00 | 57.14 |
| 455 | O | PHE | 60 | 11.895 | 0.659 | 11.609 | 1.00 | 57.14 |
| 456 | N | GLU | 61 | 11.484 | −1.345 | 12.550 | 1.00 | 62.19 |
| 457 | CA | GLU | 61 | 10.921 | −0.803 | 13.778 | 1.00 | 62.19 |
| 458 | CB | GLU | 61 | 10.401 | −1.937 | 14.671 | 1.00 | 93.34 |
| 459 | CG | GLU | 61 | 9.199 | −2.699 | 14.121 | 1.00 | 93.34 |
| 460 | CD | GLU | 61 | 9.496 | −3.449 | 12.827 | 1.00 | 93.34 |
| 461 | OE1 | GLU | 61 | 10.425 | −4.285 | 12.815 | 1.00 | 93.34 |
| 462 | OE2 | GLU | 61 | 8.792 | −3.203 | 11.823 | 1.00 | 93.34 |
| 463 | C | GLU | 61 | 11.960 | 0.013 | 14.552 | 1.00 | 62.19 |
| 464 | O | GLU | 61 | 11.609 | 0.932 | 15.285 | 1.00 | 62.19 |
| 465 | N | ASP | 62 | 13.235 | −0.315 | 14.384 | 1.00 | 48.05 |
| 466 | CA | ASP | 62 | 14.272 | 0.406 | 15.107 | 1.00 | 48.05 |
| 467 | CB | ASP | 62 | 15.573 | −0.389 | 15.119 | 1.00 | 36.56 |
| 468 | CG | ASP | 62 | 15.420 | −1.725 | 15.809 | 1.00 | 36.56 |
| 469 | OD1 | ASP | 62 | 14.696 | −1.799 | 16.822 | 1.00 | 36.56 |
| 470 | OD2 | ASP | 62 | 16.025 | −2.703 | 15.355 | 1.00 | 36.56 |
| 471 | C | ASP | 62 | 14.516 | 1.790 | 14.535 | 1.00 | 48.05 |
| 472 | O | ASP | 62 | 15.250 | 2.587 | 15.110 | 1.00 | 48.05 |
| 473 | N | SER | 63 | 13.896 | 2.076 | 13.399 | 1.00 | 39.66 |
| 474 | CA | SER | 63 | 14.076 | 3.386 | 12.809 | 1.00 | 39.66 |
| 475 | CB | SER | 63 | 13.420 | 3.454 | 11.428 | 1.00 | 51.20 |
| 476 | OG | SER | 63 | 14.091 | 2.604 | 10.524 | 1.00 | 51.20 |
| 477 | C | SER | 63 | 13.419 | 4.361 | 13.759 | 1.00 | 39.66 |
| 478 | O | SER | 63 | 12.647 | 3.966 | 14.630 | 1.00 | 39.66 |
| 479 | N | GLY | 64 | 13.722 | 5.637 | 13.613 | 1.00 | 35.00 |
| 480 | CA | GLY | 64 | 13.108 | 6.589 | 14.521 | 1.00 | 35.00 |
| 481 | C | GLY | 64 | 14.014 | 7.568 | 15.260 | 1.00 | 35.00 |
| 482 | O | GLY | 64 | 15.207 | 7.696 | 14.975 | 1.00 | 35.00 |
| 483 | N | GLU | 65 | 13.406 | 8.261 | 16.214 | 1.00 | 46.87 |
| 484 | CA | GLU | 65 | 14.075 | 9.259 | 17.020 | 1.00 | 46.87 |
| 485 | CB | GLU | 65 | 13.101 | 10.390 | 17.340 | 1.00 | 64.86 |
| 486 | CG | GLU | 65 | 13.685 | 11.460 | 18.222 | 1.00 | 64.86 |
| 487 | CD | GLU | 65 | 12.647 | 12.430 | 18.727 | 1.00 | 64.86 |
| 488 | OE1 | GLU | 65 | 11.819 | 12.030 | 19.574 | 1.00 | 64.86 |
| 489 | OE2 | GLU | 65 | 12.658 | 13.596 | 18.274 | 1.00 | 64.86 |
| 490 | C | GLU | 65 | 14.594 | 8.683 | 18.327 | 1.00 | 46.87 |
| 491 | O | GLU | 65 | 13.873 | 7.988 | 19.046 | 1.00 | 46.87 |
| 492 | N | TYR | 66 | 15.845 | 8.985 | 18.632 | 1.00 | 50.39 |
| 493 | CA | TYR | 66 | 16.453 | 8.533 | 19.882 | 1.00 | 50.39 |
| 494 | CB | TYR | 66 | 17.565 | 7.535 | 19.607 | 1.00 | 40.76 |
| 495 | CG | TYR | 66 | 17.085 | 6.188 | 19.143 | 1.00 | 40.76 |
| 496 | CD1 | TYR | 66 | 17.117 | 5.843 | 17.800 | 1.00 | 40.76 |
| 497 | CE1 | TYR | 66 | 16.746 | 4.585 | 17.382 | 1.00 | 40.76 |
| 498 | CD2 | TYR | 66 | 16.652 | 5.236 | 20.056 | 1.00 | 40.76 |
| 499 | CE2 | TYR | 66 | 16.273 | 3.973 | 19.646 | 1.00 | 40.76 |
| 500 | CZ | TYR | 66 | 16.330 | 3.653 | 18.311 | 1.00 | 40.76 |
| 501 | OH | TYR | 66 | 16.024 | 2.378 | 17.910 | 1.00 | 40.76 |
| 502 | C | TYR | 66 | 17.032 | 9.711 | 20.658 | 1.00 | 50.39 |
| 503 | O | TYR | 66 | 17.732 | 10.535 | 20.075 | 1.00 | 50.39 |
| 504 | N | LYS | 67 | 16.719 | 9.785 | 21.955 | 1.00 | 55.49 |
| 505 | CA | LYS | 67 | 17.221 | 10.837 | 22.854 | 1.00 | 55.49 |
| 506 | CB | LYS | 67 | 16.106 | 11.762 | 23.379 | 1.00 | 68.70 |
| 507 | CG | LYS | 67 | 15.118 | 12.362 | 22.412 | 1.00 | 68.70 |
| 508 | CD | LYS | 67 | 13.879 | 12.767 | 23.199 | 1.00 | 68.70 |
| 509 | CE | LYS | 67 | 12.818 | 13.407 | 22.338 | 1.00 | 68.70 |
| 510 | NZ | LYS | 67 | 11.597 | 13.688 | 23.149 | 1.00 | 68.70 |
| 511 | C | LYS | 67 | 17.734 | 10.114 | 24.092 | 1.00 | 55.49 |
| 512 | O | LYS | 67 | 17.209 | 9.054 | 24.446 | 1.00 | 55.49 |
| 513 | N | CYS | 68 | 18.749 | 10.658 | 24.759 | 1.00 | 60.89 |
| 514 | CA | CYS | 68 | 19.179 | 10.011 | 25.992 | 1.00 | 60.89 |
| 515 | C | CYS | 68 | 19.028 | 10.988 | 27.145 | 1.00 | 60.89 |
| 516 | O | CYS | 68 | 18.795 | 12.193 | 26.946 | 1.00 | 60.89 |
| 517 | CB | CYS | 68 | 20.594 | 9.418 | 25.897 | 1.00 | 63.38 |
| 518 | SG | CYS | 68 | 22.069 | 10.482 | 25.959 | 1.00 | 63.38 |
| 519 | N | GLN | 69 | 19.113 | 10.457 | 28.355 | 1.00 | 61.40 |
| 520 | CA | GLN | 69 | 18.943 | 11.268 | 29.546 | 1.00 | 61.40 |
| 521 | CB | GLN | 69 | 17.495 | 11.161 | 29.998 | 1.00 | 108.41 |
| 522 | CG | GLN | 69 | 17.207 | 11.587 | 31.426 | 1.00 | 108.41 |
| 523 | CD | GLN | 69 | 16.245 | 12.754 | 31.486 | 1.00 | 108.41 |
| 524 | OE1 | GLN | 69 | 15.641 | 13.126 | 30.493 | 1.00 | 108.41 |
| 525 | NE2 | GLN | 69 | 16.097 | 13.333 | 32.665 | 1.00 | 108.41 |
| 526 | C | GLN | 69 | 19.858 | 10.792 | 30.642 | 1.00 | 61.40 |
| 527 | O | GLN | 69 | 19.859 | 9.609 | 31.005 | 1.00 | 61.40 |
| 528 | N | HIS | 70 | 20.653 | 11.729 | 31.139 | 1.00 | 103.97 |
| 529 | CA | HIS | 70 | 21.594 | 11.480 | 32.217 | 1.00 | 103.97 |
| 530 | CB | HIS | 70 | 23.011 | 11.824 | 31.761 | 1.00 | 140.89 |
| 531 | CG | HIS | 70 | 24.032 | 11.697 | 32.844 | 1.00 | 140.89 |
| 532 | CD2 | HIS | 70 | 24.744 | 12.638 | 33.503 | 1.00 | 140.89 |
| 533 | ND1 | HIS | 70 | 24.407 | 10.485 | 33.376 | 1.00 | 140.89 |
| 534 | CE1 | HIS | 70 | 25.311 | 10.685 | 34.319 | 1.00 | 140.89 |
| 535 | NE2 | HIS | 70 | 25.534 | 11.984 | 34.416 | 1.00 | 140.89 |
| 536 | C | HIS | 70 | 21.186 | 12.373 | 33.396 | 1.00 | 103.97 |
| 537 | O | HIS | 70 | 20.184 | 13.088 | 33.319 | 1.00 | 103.97 |
| 538 | N | GLN | 71 | 21.968 | 12.364 | 34.470 | 1.00 | 101.70 |
| 539 | CA | GLN | 71 | 21.646 | 13.163 | 35.640 | 1.00 | 101.70 |
| 540 | CB | GLN | 71 | 22.512 | 12.706 | 36.820 | 1.00 | 136.43 |
| 541 | CG | GLN | 71 | 21.739 | 11.968 | 37.924 | 1.00 | 136.43 |
| 542 | CD | GLN | 71 | 22.639 | 11.194 | 38.876 | 1.00 | 136.43 |
| 543 | OE1 | GLN | 71 | 23.660 | 11.696 | 39.322 | 1.00 | 136.43 |
| 544 | NE2 | GLN | 71 | 22.242 | 9.966 | 39.206 | 1.00 | 136.43 |
| 545 | C | GLN | 71 | 21.782 | 14.663 | 35.407 | 1.00 | 101.70 |
| 546 | O | GLN | 71 | 21.838 | 15.441 | 36.361 | 1.00 | 101.70 |
| 547 | N | GLN | 72 | 21.778 | 15.065 | 34.137 | 1.00 | 141.76 |
| 548 | CA | GLN | 72 | 21.905 | 16.470 | 33.755 | 1.00 | 141.76 |
| 549 | CB | GLN | 72 | 22.748 | 16.587 | 32.494 | 1.00 | 141.59 |
| 550 | CG | GLN | 72 | 24.182 | 16.131 | 32.710 | 1.00 | 141.59 |
| 551 | CD | GLN | 72 | 25.045 | 16.307 | 31.482 | 1.00 | 141.59 |
| 552 | OE1 | GLN | 72 | 24.616 | 16.858 | 30.472 | 1.00 | 141.59 |
| 553 | NE2 | GLN | 72 | 26.285 | 15.841 | 31.570 | 1.00 | 141.59 |
| 554 | C | GLN | 72 | 20.578 | 17.187 | 33.541 | 1.00 | 141.76 |
| 555 | O | GLN | 72 | 20.531 | 18.246 | 32.918 | 1.00 | 141.76 |
| 556 | N | VAL | 73 | 19.509 | 16.598 | 34.067 | 1.00 | 141.76 |
| 557 | CA | VAL | 73 | 18.150 | 17.144 | 33.996 | 1.00 | 141.76 |
| 558 | CB | VAL | 73 | 17.945 | 18.230 | 35.093 | 1.00 | 113.45 |
| 559 | CG1 | VAL | 73 | 16.471 | 18.593 | 35.215 | 1.00 | 113.45 |
| 560 | CG2 | VAL | 73 | 18.475 | 17.718 | 36.424 | 1.00 | 113.45 |
| 561 | C | VAL | 73 | 17.677 | 17.707 | 32.644 | 1.00 | 141.76 |
| 562 | O | VAL | 73 | 16.643 | 18.376 | 32.568 | 1.00 | 141.76 |
| 563 | N | ASN | 74 | 18.433 | 17.441 | 31.583 | 1.00 | 110.03 |
| 564 | CA | ASN | 74 | 18.056 | 17.906 | 30.249 | 1.00 | 110.03 |
| 565 | CB | ASN | 74 | 18.782 | 19.208 | 29.880 | 1.00 | 123.79 |
| 566 | CG | ASN | 74 | 18.013 | 20.459 | 30.295 | 1.00 | 123.79 |
| 567 | OD1 | ASN | 74 | 16.818 | 20.402 | 30.597 | 1.00 | 123.79 |
| 568 | ND2 | ASN | 74 | 18.698 | 21.598 | 30.291 | 1.00 | 123.79 |
| 569 | C | ASN | 74 | 18.382 | 16.840 | 29.212 | 1.00 | 110.03 |
| 570 | O | ASN | 74 | 19.531 | 16.412 | 29.094 | 1.00 | 110.03 |
| 571 | N | GLU | 75 | 17.367 | 16.416 | 28.460 | 1.00 | 64.12 |
| 572 | CA | GLU | 75 | 17.552 | 15.399 | 27.433 | 1.00 | 64.12 |
| 573 | CB | GLU | 75 | 16.190 | 14.961 | 26.882 | 1.00 | 93.37 |
| 574 | CG | GLU | 75 | 15.332 | 14.330 | 27.971 | 1.00 | 93.37 |
| 575 | CD | GLU | 75 | 13.963 | 13.868 | 27.511 | 1.00 | 93.37 |
| 576 | OE1 | GLU | 75 | 13.583 | 14.146 | 26.355 | 1.00 | 93.37 |
| 577 | OE2 | GLU | 75 | 13.264 | 13.227 | 28.327 | 1.00 | 93.37 |
| 578 | C | GLU | 75 | 18.454 | 15.910 | 26.319 | 1.00 | 64.12 |
| 579 | O | GLU | 75 | 18.591 | 17.118 | 26.120 | 1.00 | 64.12 |
| 580 | N | SER | 76 | 19.090 | 14.984 | 25.611 | 1.00 | 56.18 |
| 581 | CA | SER | 76 | 19.980 | 15.362 | 24.518 | 1.00 | 56.18 |
| 582 | CB | SER | 76 | 20.944 | 14.217 | 24.188 | 1.00 | 74.86 |
| 583 | OG | SER | 76 | 20.235 | 13.084 | 23.720 | 1.00 | 74.86 |
| 584 | C | SER | 76 | 19.189 | 15.720 | 23.263 | 1.00 | 56.18 |
| 585 | O | SER | 76 | 17.968 | 15.559 | 23.199 | 1.00 | 56.18 |
| 586 | N | GLU | 77 | 19.896 | 16.237 | 22.271 | 1.00 | 67.41 |
| 587 | CA | GLU | 77 | 19.250 | 16.561 | 21.021 | 1.00 | 67.41 |
| 588 | CB | GLU | 77 | 20.205 | 17.326 | 20.096 | 1.00 | 96.50 |
| 589 | CG | GLU | 77 | 20.530 | 18.734 | 20.563 | 1.00 | 96.50 |
| 590 | CD | GLU | 77 | 19.281 | 19.545 | 20.859 | 1.00 | 96.50 |
| 591 | OE1 | GLU | 77 | 18.406 | 19.638 | 19.973 | 1.00 | 96.50 |
| 592 | OE2 | GLU | 77 | 19.173 | 20.089 | 21.978 | 1.00 | 96.50 |
| 593 | C | GLU | 77 | 18.903 | 15.205 | 20.416 | 1.00 | 67.41 |
| 594 | O | GLU | 77 | 19.605 | 14.210 | 20.646 | 1.00 | 67.41 |
| 595 | N | PRO | 78 | 17.805 | 15.136 | 19.660 | 1.00 | 64.74 |
| 596 | CD | PRO | 78 | 16.712 | 16.106 | 19.495 | 1.00 | 62.17 |
| 597 | CA | PRO | 78 | 17.447 | 13.852 | 19.067 | 1.00 | 64.74 |
| 598 | CB | PRO | 78 | 16.047 | 14.101 | 18.514 | 1.00 | 62.17 |
| 599 | CG | PRO | 78 | 15.527 | 15.197 | 19.372 | 1.00 | 62.17 |
| 600 | C | PRO | 78 | 18.421 | 13.473 | 17.959 | 1.00 | 64.74 |
| 601 | O | PRO | 78 | 19.118 | 14.321 | 17.404 | 1.00 | 64.74 |
| 602 | N | VAL | 79 | 18.469 | 12.183 | 17.670 | 1.00 | 45.82 |
| 603 | CA | VAL | 79 | 19.274 | 11.638 | 16.593 | 1.00 | 45.82 |
| 604 | CB | VAL | 79 | 20.455 | 10.798 | 17.123 | 1.00 | 63.76 |
| 605 | CG1 | VAL | 79 | 21.165 | 10.108 | 15.972 | 1.00 | 63.76 |
| 606 | CG2 | VAL | 79 | 21.437 | 11.702 | 17.841 | 1.00 | 63.76 |
| 607 | C | VAL | 79 | 18.266 | 10.745 | 15.857 | 1.00 | 45.82 |
| 608 | O | VAL | 79 | 17.396 | 10.128 | 16.485 | 1.00 | 45.82 |
| 609 | N | TYR | 80 | 18.347 | 10.697 | 14.533 | 1.00 | 52.05 |
| 610 | CA | TYR | 80 | 17.396 | 9.886 | 13.790 | 1.00 | 52.05 |
| 611 | CB | TYR | 80 | 16.612 | 10.760 | 12.810 | 1.00 | 70.93 |
| 612 | CG | TYR | 80 | 15.767 | 11.787 | 13.520 | 1.00 | 70.93 |
| 613 | CD1 | TYR | 80 | 16.329 | 12.975 | 13.998 | 1.00 | 70.93 |
| 614 | CE1 | TYR | 80 | 15.573 | 13.882 | 14.744 | 1.00 | 70.93 |
| 615 | CD2 | TYR | 80 | 14.422 | 11.533 | 13.798 | 1.00 | 70.93 |
| 616 | CE2 | TYR | 80 | 13.656 | 12.427 | 14.543 | 1.00 | 70.93 |
| 617 | CZ | TYR | 80 | 14.237 | 13.598 | 15.017 | 1.00 | 70.93 |
| 618 | OH | TYR | 80 | 13.493 | 14.459 | 15.798 | 1.00 | 70.93 |
| 619 | C | TYR | 80 | 18.016 | 8.711 | 13.074 | 1.00 | 52.05 |
| 620 | O | TYR | 80 | 18.975 | 8.859 | 12.320 | 1.00 | 52.05 |
| 621 | N | LEU | 81 | 17.464 | 7.532 | 13.332 | 1.00 | 46.77 |
| 622 | CA | LEU | 81 | 17.960 | 6.319 | 12.702 | 1.00 | 46.77 |
| 623 | CB | LEU | 81 | 18.213 | 5.249 | 13.767 | 1.00 | 49.82 |
| 624 | CG | LEU | 81 | 19.042 | 4.032 | 13.338 | 1.00 | 49.82 |
| 625 | CD1 | LEU | 81 | 20.515 | 4.427 | 13.231 | 1.00 | 49.82 |
| 626 | CD2 | LEU | 81 | 18.884 | 2.907 | 14.354 | 1.00 | 49.82 |
| 627 | C | LEU | 81 | 16.935 | 5.811 | 11.659 | 1.00 | 46.77 |
| 628 | O | LEU | 81 | 15.720 | 5.865 | 11.881 | 1.00 | 46.77 |
| 629 | N | GLU | 82 | 17.434 | 5.337 | 10.523 | 1.00 | 44.84 |
| 630 | CA | GLU | 82 | 16.568 | 4.816 | 9.487 | 1.00 | 44.84 |
| 631 | CB | GLU | 82 | 16.372 | 5.863 | 8.395 | 1.00 | 75.78 |
| 632 | CG | GLU | 82 | 15.459 | 5.412 | 7.277 | 1.00 | 75.78 |
| 633 | CD | GLU | 82 | 14.890 | 6.571 | 6.490 | 1.00 | 75.78 |
| 634 | OE1 | GLU | 82 | 15.573 | 7.613 | 6.387 | 1.00 | 75.78 |
| 635 | OE2 | GLU | 82 | 13.764 | 6.437 | 5.968 | 1.00 | 75.78 |
| 636 | C | GLU | 82 | 17.124 | 3.526 | 8.900 | 1.00 | 44.84 |
| 637 | O | GLU | 82 | 18.256 | 3.478 | 8.434 | 1.00 | 44.84 |
| 638 | N | VAL | 83 | 16.308 | 2.482 | 8.915 | 1.00 | 58.13 |
| 639 | CA | VAL | 83 | 16.715 | 1.177 | 8.410 | 1.00 | 58.13 |
| 640 | CB | VAL | 83 | 16.204 | 0.070 | 9.358 | 1.00 | 48.46 |
| 641 | CG1 | VAL | 83 | 16.693 | −1.285 | 8.909 | 1.00 | 48.46 |
| 642 | CG2 | VAL | 83 | 16.668 | 0.369 | 10.783 | 1.00 | 48.46 |
| 643 | C | VAL | 83 | 16.212 | 0.919 | 6.986 | 1.00 | 58.13 |
| 644 | O | VAL | 83 | 15.033 | 1.088 | 6.697 | 1.00 | 58.13 |
| 645 | N | PHE | 84 | 17.118 | 0.509 | 6.103 | 1.00 | 54.77 |
| 646 | CA | PHE | 84 | 16.772 | 0.235 | 4.720 | 1.00 | 54.77 |
| 647 | CB | PHE | 84 | 17.572 | 1.115 | 3.750 | 1.00 | 54.95 |
| 648 | CG | PHE | 84 | 17.424 | 2.584 | 3.986 | 1.00 | 54.95 |
| 649 | CD1 | PHE | 84 | 18.222 | 3.227 | 4.937 | 1.00 | 54.95 |
| 650 | CD2 | PHE | 84 | 16.503 | 3.336 | 3.246 | 1.00 | 54.95 |
| 651 | CE1 | PHE | 84 | 18.111 | 4.600 | 5.143 | 1.00 | 54.95 |
| 652 | CE2 | PHE | 84 | 16.378 | 4.708 | 3.438 | 1.00 | 54.95 |
| 653 | CZ | PHE | 84 | 17.185 | 5.349 | 4.388 | 1.00 | 54.95 |
| 654 | C | PHE | 84 | 17.031 | −1.194 | 4.305 | 1.00 | 54.77 |
| 655 | O | PHE | 84 | 17.743 | −1.947 | 4.980 | 1.00 | 54.77 |
| 656 | N | SER | 85 | 16.474 | −1.527 | 3.148 | 1.00 | 50.72 |
| 657 | CA | SER | 85 | 16.625 | −2.831 | 2.519 | 1.00 | 50.72 |
| 658 | CB | SER | 85 | 15.392 | −3.696 | 2.776 | 1.00 | 85.41 |
| 659 | OG | SER | 85 | 15.578 | −4.996 | 2.253 | 1.00 | 85.41 |
| 660 | C | SER | 85 | 16.737 | −2.509 | 1.031 | 1.00 | 50.72 |
| 661 | O | SER | 85 | 15.741 | −2.166 | 0.397 | 1.00 | 50.72 |
| 662 | N | ASP | 86 | 17.933 | −2.595 | 0.470 | 1.00 | 46.74 |
| 663 | CA | ASP | 86 | 18.122 | −2.283 | −0.939 | 1.00 | 46.74 |
| 664 | CB | ASP | 86 | 18.070 | −0.766 | −1.139 | 1.00 | 57.20 |
| 665 | CG | ASP | 86 | 17.810 | −0.345 | −2.581 | 1.00 | 57.20 |
| 666 | OD1 | ASP | 86 | 18.547 | −0.781 | −3.500 | 1.00 | 57.20 |
| 667 | OD2 | ASP | 86 | 16.866 | 0.442 | −2.795 | 1.00 | 57.20 |
| 668 | C | ASP | 86 | 19.499 | −2.821 | −1.277 | 1.00 | 46.74 |
| 669 | O | ASP | 86 | 20.166 | −3.402 | −0.429 | 1.00 | 46.74 |
| 670 | N | TRP | 87 | 19.936 | −2.615 | −2.505 | 1.00 | 48.74 |
| 671 | CA | TRP | 87 | 21.241 | −3.073 | −2.935 | 1.00 | 48.74 |
| 672 | CB | TRP | 87 | 21.226 | −3.366 | −4.440 | 1.00 | 51.62 |
| 673 | CG | TRP | 87 | 20.649 | −4.704 | −4.804 | 1.00 | 51.62 |
| 674 | CD2 | TRP | 87 | 19.258 | −5.039 | −4.934 | 1.00 | 51.62 |
| 675 | CE2 | TRP | 87 | 19.191 | −6.406 | −5.285 | 1.00 | 51.62 |
| 676 | CE3 | TRP | 87 | 18.064 | −4.316 | −4.793 | 1.00 | 51.62 |
| 677 | CD1 | TRP | 87 | 21.344 | −5.846 | −5.072 | 1.00 | 51.62 |
| 678 | NE1 | TRP | 87 | 20.479 | −6.872 | −5.361 | 1.00 | 51.62 |
| 679 | CZ2 | TRP | 87 | 17.966 | −7.069 | −5.500 | 1.00 | 51.62 |
| 680 | CZ3 | TRP | 87 | 16.849 | −4.974 | −5.006 | 1.00 | 51.62 |
| 681 | CH2 | TRP | 87 | 16.813 | −6.337 | −5.357 | 1.00 | 51.62 |
| 682 | C | TRP | 87 | 22.285 | −2.011 | −2.634 | 1.00 | 48.74 |
| 683 | O | TRP | 87 | 23.440 | −2.327 | −2.297 | 1.00 | 48.74 |
| 684 | N | LEU | 88 | 21.889 | −0.752 | −2.793 | 1.00 | 48.62 |
| 685 | CA | LEU | 88 | 22.774 | 0.361 | −2.517 | 1.00 | 48.62 |
| 686 | CB | LEU | 88 | 23.101 | 1.159 | −3.775 | 1.00 | 41.58 |
| 687 | CG | LEU | 88 | 24.163 | 0.628 | −4.731 | 1.00 | 41.58 |
| 688 | CD1 | LEU | 88 | 24.506 | 1.715 | −5.724 | 1.00 | 41.58 |
| 689 | CD2 | LEU | 88 | 25.415 | 0.208 | −3.975 | 1.00 | 41.58 |
| 690 | C | LEU | 88 | 22.130 | 1.290 | −1.503 | 1.00 | 48.62 |
| 691 | O | LEU | 88 | 20.909 | 1.448 | −1.458 | 1.00 | 48.62 |
| 692 | N | LEU | 89 | 22.974 | 1.895 | −0.683 | 1.00 | 46.32 |
| 693 | CA | LEU | 89 | 22.532 | 2.823 | 0.336 | 1.00 | 46.32 |
| 694 | CB | LEU | 89 | 22.342 | 2.098 | 1.671 | 1.00 | 41.30 |
| 695 | CG | LEU | 89 | 22.014 | 2.940 | 2.913 | 1.00 | 41.30 |
| 696 | CD1 | LEU | 89 | 20.786 | 3.782 | 2.656 | 1.00 | 41.30 |
| 697 | CD2 | LEU | 89 | 21.787 | 2.032 | 4.113 | 1.00 | 41.30 |
| 698 | C | LEU | 89 | 23.636 | 3.863 | 0.459 | 1.00 | 46.32 |
| 699 | O | LEU | 89 | 24.821 | 3.509 | 0.602 | 1.00 | 46.32 |
| 700 | N | LEU | 90 | 23.259 | 5.134 | 0.353 | 1.00 | 43.08 |
| 701 | CA | LEU | 90 | 24.233 | 6.196 | 0.489 | 1.00 | 43.08 |
| 702 | CB | LEU | 90 | 23.818 | 7.418 | −0.318 | 1.00 | 46.82 |
| 703 | CG | LEU | 90 | 24.810 | 8.588 | −0.299 | 1.00 | 46.82 |
| 704 | CD1 | LEU | 90 | 26.217 | 8.116 | −0.656 | 1.00 | 46.82 |
| 705 | CD2 | LEU | 90 | 24.344 | 9.655 | −1.270 | 1.00 | 46.82 |
| 706 | C | LEU | 90 | 24.229 | 6.528 | 1.975 | 1.00 | 43.08 |
| 707 | O | LEU | 90 | 23.177 | 6.760 | 2.571 | 1.00 | 43.08 |
| 708 | N | GLN | 91 | 25.404 | 6.493 | 2.588 | 1.00 | 44.37 |
| 709 | CA | GLN | 91 | 25.484 | 6.817 | 4.000 | 1.00 | 44.37 |
| 710 | CB | GLN | 91 | 26.177 | 5.695 | 4.753 | 1.00 | 39.09 |
| 711 | CG | GLN | 91 | 25.435 | 4.377 | 4.730 | 1.00 | 39.09 |
| 712 | CD | GLN | 91 | 26.190 | 3.286 | 5.468 | 1.00 | 39.09 |
| 713 | OE1 | GLN | 91 | 27.337 | 2.992 | 5.162 | 1.00 | 39.09 |
| 714 | NE2 | GLN | 91 | 25.535 | 2.678 | 6.433 | 1.00 | 39.09 |
| 715 | C | GLN | 91 | 26.261 | 8.121 | 4.136 | 1.00 | 44.37 |
| 716 | O | GLN | 91 | 27.172 | 8.390 | 3.357 | 1.00 | 44.37 |
| 717 | N | ALA | 92 | 25.860 | 8.948 | 5.091 | 1.00 | 44.40 |
| 718 | CA | ALA | 92 | 26.534 | 10.217 | 5.309 | 1.00 | 44.40 |
| 719 | CB | ALA | 92 | 25.618 | 11.365 | 4.952 | 1.00 | 35.90 |
| 720 | C | ALA | 92 | 26.921 | 10.332 | 6.767 | 1.00 | 44.40 |
| 721 | O | ALA | 92 | 26.223 | 9.806 | 7.631 | 1.00 | 44.40 |
| 722 | N | SER | 93 | 28.025 | 11.019 | 7.041 | 1.00 | 39.65 |
| 723 | CA | SER | 93 | 28.435 | 11.214 | 8.419 | 1.00 | 39.65 |
| 724 | CB | SER | 93 | 29.821 | 11.866 | 8.493 | 1.00 | 42.29 |
| 725 | OG | SER | 93 | 29.947 | 12.998 | 7.649 | 1.00 | 42.29 |
| 726 | C | SER | 93 | 27.373 | 12.092 | 9.062 | 1.00 | 39.65 |
| 727 | O | SER | 93 | 27.048 | 11.939 | 10.239 | 1.00 | 39.65 |
| 728 | N | ALA | 94 | 26.801 | 13.006 | 8.291 | 1.00 | 49.05 |
| 729 | CA | ALA | 94 | 25.759 | 13.865 | 8.848 | 1.00 | 49.05 |
| 730 | CB | ALA | 94 | 26.397 | 15.014 | 9.617 | 1.00 | 25.45 |
| 731 | C | ALA | 94 | 24.815 | 14.416 | 7.775 | 1.00 | 49.05 |
| 732 | O | ALA | 94 | 25.238 | 14.702 | 6.668 | 1.00 | 49.05 |
| 733 | N | GLU | 95 | 23.542 | 14.563 | 8.099 | 1.00 | 54.30 |
| 734 | CA | GLU | 95 | 22.598 | 15.115 | 7.126 | 1.00 | 54.30 |
| 735 | CB | GLU | 95 | 21.200 | 14.552 | 7.355 | 1.00 | 64.16 |
| 736 | CG | GLU | 95 | 21.107 | 13.088 | 6.999 | 1.00 | 64.16 |
| 737 | CD | GLU | 95 | 19.770 | 12.488 | 7.345 | 1.00 | 64.16 |
| 738 | OE1 | GLU | 95 | 19.563 | 11.286 | 7.063 | 1.00 | 64.16 |
| 739 | OE2 | GLU | 95 | 18.926 | 13.218 | 7.899 | 1.00 | 64.16 |
| 740 | C | GLU | 95 | 22.594 | 16.636 | 7.253 | 1.00 | 54.30 |
| 741 | O | GLU | 95 | 22.044 | 17.332 | 6.405 | 1.00 | 54.30 |
| 742 | N | VAL | 96 | 23.234 | 17.127 | 8.317 | 1.00 | 54.64 |
| 743 | CA | VAL | 96 | 23.366 | 18.550 | 8.595 | 1.00 | 54.64 |
| 744 | CB | VAL | 96 | 22.414 | 19.000 | 9.707 | 1.00 | 44.67 |
| 745 | CG1 | VAL | 96 | 22.662 | 20.472 | 10.030 | 1.00 | 44.67 |
| 746 | CG2 | VAL | 96 | 20.957 | 18.784 | 9.274 | 1.00 | 44.67 |
| 747 | C | VAL | 96 | 24.800 | 18.787 | 9.049 | 1.00 | 54.64 |
| 748 | O | VAL | 96 | 25.161 | 18.494 | 10.194 | 1.00 | 54.64 |
| 749 | N | VAL | 97 | 25.613 | 19.306 | 8.134 | 1.00 | 48.96 |
| 750 | CA | VAL | 97 | 27.023 | 19.592 | 8.365 | 1.00 | 48.96 |
| 751 | CB | VAL | 97 | 27.866 | 19.209 | 7.133 | 1.00 | 46.14 |
| 752 | CG1 | VAL | 97 | 29.339 | 19.450 | 7.407 | 1.00 | 46.14 |
| 753 | CG2 | VAL | 97 | 27.628 | 17.773 | 6.764 | 1.00 | 46.14 |
| 754 | C | VAL | 97 | 27.257 | 21.078 | 8.619 | 1.00 | 48.96 |
| 755 | O | VAL | 97 | 26.654 | 21.934 | 7.956 | 1.00 | 48.96 |
| 756 | N | MET | 98 | 28.148 | 21.385 | 9.560 | 1.00 | 48.67 |
| 757 | CA | MET | 98 | 28.479 | 22.770 | 9.866 | 1.00 | 48.67 |
| 758 | CB | MET | 98 | 28.895 | 22.920 | 11.329 | 1.00 | 88.16 |
| 759 | CG | MET | 98 | 27.724 | 22.792 | 12.290 | 1.00 | 88.16 |
| 760 | SD | MET | 98 | 28.143 | 23.133 | 14.001 | 1.00 | 88.16 |
| 761 | CE | MET | 98 | 28.281 | 21.471 | 14.634 | 1.00 | 88.16 |
| 762 | C | MET | 98 | 29.592 | 23.222 | 8.937 | 1.00 | 48.67 |
| 763 | O | MET | 98 | 30.487 | 22.451 | 8.603 | 1.00 | 48.67 |
| 764 | N | GLU | 99 | 29.516 | 24.467 | 8.488 | 1.00 | 51.28 |
| 765 | CA | GLU | 99 | 30.518 | 25.003 | 7.579 | 1.00 | 51.28 |
| 766 | CB | GLU | 99 | 30.227 | 26.488 | 7.339 | 1.00 | 66.75 |
| 767 | CG | GLU | 99 | 31.195 | 27.175 | 6.403 | 1.00 | 66.75 |
| 768 | CD | GLU | 99 | 30.842 | 28.632 | 6.164 | 1.00 | 66.75 |
| 769 | OE1 | GLU | 99 | 30.515 | 29.334 | 7.146 | 1.00 | 66.75 |
| 770 | OE2 | GLU | 99 | 30.901 | 29.072 | 4.994 | 1.00 | 66.75 |
| 771 | C | GLU | 99 | 31.939 | 24.801 | 8.133 | 1.00 | 51.28 |
| 772 | O | GLU | 99 | 32.182 | 25.000 | 9.318 | 1.00 | 51.28 |
| 773 | N | GLY | 100 | 32.874 | 24.401 | 7.280 | 1.00 | 56.63 |
| 774 | CA | GLY | 100 | 34.233 | 24.185 | 7.748 | 1.00 | 56.63 |
| 775 | C | GLY | 100 | 34.534 | 22.752 | 8.172 | 1.00 | 56.63 |
| 776 | O | GLY | 100 | 35.670 | 22.300 | 8.074 | 1.00 | 56.63 |
| 777 | N | GLN | 101 | 33.519 | 22.037 | 8.654 | 1.00 | 64.74 |
| 778 | CA | GLN | 101 | 33.683 | 20.645 | 9.065 | 1.00 | 64.74 |
| 779 | CB | GLN | 101 | 32.550 | 20.236 | 10.003 | 1.00 | 77.71 |
| 780 | CG | GLN | 101 | 32.559 | 20.959 | 11.325 | 1.00 | 77.71 |
| 781 | CD | GLN | 101 | 33.844 | 20.719 | 12.090 | 1.00 | 77.71 |
| 782 | OE1 | GLN | 101 | 34.912 | 21.200 | 11.702 | 1.00 | 77.71 |
| 783 | NE2 | GLN | 101 | 33.751 | 19.957 | 13.179 | 1.00 | 77.71 |
| 784 | C | GLN | 101 | 33.695 | 19.702 | 7.855 | 1.00 | 64.74 |
| 785 | O | GLN | 101 | 33.327 | 20.083 | 6.745 | 1.00 | 64.74 |
| 786 | N | PRO | 102 | 34.132 | 18.452 | 8.055 | 1.00 | 53.53 |
| 787 | CD | PRO | 102 | 34.809 | 17.877 | 9.236 | 1.00 | 44.31 |
| 788 | CA | PRO | 102 | 34.163 | 17.510 | 6.936 | 1.00 | 53.53 |
| 789 | CB | PRO | 102 | 35.317 | 16.590 | 7.311 | 1.00 | 44.31 |
| 790 | CG | PRO | 102 | 35.097 | 16.438 | 8.790 | 1.00 | 44.31 |
| 791 | C | PRO | 102 | 32.841 | 16.752 | 6.795 | 1.00 | 53.53 |
| 792 | O | PRO | 102 | 32.064 | 16.635 | 7.739 | 1.00 | 53.53 |
| 793 | N | LEU | 103 | 32.604 | 16.242 | 5.596 | 1.00 | 42.44 |
| 794 | CA | LEU | 103 | 31.403 | 15.480 | 5.300 | 1.00 | 42.44 |
| 795 | CB | LEU | 103 | 30.500 | 16.243 | 4.317 | 1.00 | 43.14 |
| 796 | CG | LEU | 103 | 29.332 | 15.406 | 3.768 | 1.00 | 43.14 |
| 797 | CD1 | LEU | 103 | 28.454 | 14.974 | 4.920 | 1.00 | 43.14 |
| 798 | CD2 | LEU | 103 | 28.522 | 16.193 | 2.768 | 1.00 | 43.14 |
| 799 | C | LEU | 103 | 31.845 | 14.169 | 4.673 | 1.00 | 42.44 |
| 800 | O | LEU | 103 | 32.569 | 14.163 | 3.679 | 1.00 | 42.44 |
| 801 | N | PHE | 104 | 31.429 | 13.052 | 5.246 | 1.00 | 40.42 |
| 802 | CA | PHE | 104 | 31.825 | 11.772 | 4.659 | 1.00 | 40.42 |
| 803 | CB | PHE | 104 | 32.503 | 10.860 | 5.701 | 1.00 | 57.95 |
| 804 | CG | PHE | 104 | 33.631 | 11.515 | 6.453 | 1.00 | 57.95 |
| 805 | CD1 | PHE | 104 | 33.383 | 12.232 | 7.625 | 1.00 | 57.95 |
| 806 | CD2 | PHE | 104 | 34.943 | 11.411 | 6.003 | 1.00 | 57.95 |
| 807 | CE1 | PHE | 104 | 34.436 | 12.836 | 8.341 | 1.00 | 57.95 |
| 808 | CE2 | PHE | 104 | 35.999 | 12.011 | 6.714 | 1.00 | 57.95 |
| 809 | CZ | PHE | 104 | 35.741 | 12.721 | 7.880 | 1.00 | 57.95 |
| 810 | C | PHE | 104 | 30.635 | 11.025 | 4.045 | 1.00 | 40.42 |
| 811 | O | PHE | 104 | 29.571 | 10.908 | 4.661 | 1.00 | 40.42 |
| 812 | N | LEU | 105 | 30.837 | 10.528 | 2.833 | 1.00 | 44.12 |
| 813 | CA | LEU | 105 | 29.810 | 9.765 | 2.144 | 1.00 | 44.12 |
| 814 | CB | LEU | 105 | 29.383 | 10.445 | 0.840 | 1.00 | 40.26 |
| 815 | CG | LEU | 105 | 28.872 | 11.866 | 0.989 | 1.00 | 40.26 |
| 816 | CD1 | LEU | 105 | 28.555 | 12.435 | −0.367 | 1.00 | 40.26 |
| 817 | CD2 | LEU | 105 | 27.668 | 11.857 | 1.899 | 1.00 | 40.26 |
| 818 | C | LEU | 105 | 30.382 | 8.393 | 1.829 | 1.00 | 44.12 |
| 819 | O | LEU | 105 | 31.587 | 8.227 | 1.639 | 1.00 | 44.12 |
| 820 | N | ARG | 106 | 29.497 | 7.416 | 1.745 | 1.00 | 45.34 |
| 821 | CA | ARG | 106 | 29.912 | 6.057 | 1.484 | 1.00 | 45.34 |
| 822 | CB | ARG | 106 | 30.135 | 5.384 | 2.829 | 1.00 | 57.54 |
| 823 | CG | ARG | 106 | 30.591 | 3.964 | 2.800 | 1.00 | 57.54 |
| 824 | CD | ARG | 106 | 30.297 | 3.378 | 4.162 | 1.00 | 57.54 |
| 825 | NE | ARG | 106 | 30.888 | 2.066 | 4.356 | 1.00 | 57.54 |
| 826 | CZ | ARG | 106 | 30.501 | 1.212 | 5.297 | 1.00 | 57.54 |
| 827 | NH1 | ARG | 106 | 29.516 | 1.530 | 6.128 | 1.00 | 57.54 |
| 828 | NH2 | ARG | 106 | 31.110 | 0.040 | 5.407 | 1.00 | 57.54 |
| 829 | C | ARG | 106 | 28.800 | 5.359 | 0.710 | 1.00 | 45.34 |
| 830 | O | ARG | 106 | 27.658 | 5.345 | 1.160 | 1.00 | 45.34 |
| 831 | N | CYS | 107 | 29.129 | 4.824 | −0.465 | 1.00 | 48.19 |
| 832 | CA | CYS | 107 | 28.156 | 4.098 | −1.275 | 1.00 | 48.19 |
| 833 | C | CYS | 107 | 28.337 | 2.663 | −0.779 | 1.00 | 48.19 |
| 834 | O | CYS | 107 | 29.268 | 1.948 | −1.166 | 1.00 | 48.19 |
| 835 | CB | CYS | 107 | 28.474 | 4.238 | −2.769 | 1.00 | 54.03 |
| 836 | SG | CYS | 107 | 27.089 | 3.836 | −3.893 | 1.00 | 54.03 |
| 837 | N | HIS | 108 | 27.430 | 2.286 | 0.116 | 1.00 | 52.57 |
| 838 | CA | HIS | 108 | 27.433 | 1.009 | 0.797 | 1.00 | 52.57 |
| 839 | CB | HIS | 108 | 26.986 | 1.261 | 2.243 | 1.00 | 42.83 |
| 840 | CG | HIS | 108 | 27.086 | 0.067 | 3.134 | 1.00 | 42.83 |
| 841 | CD2 | HIS | 108 | 26.211 | −0.448 | 4.029 | 1.00 | 42.83 |
| 842 | ND1 | HIS | 108 | 28.214 | −0.725 | 3.196 | 1.00 | 42.83 |
| 843 | CE1 | HIS | 108 | 28.026 | −1.677 | 4.092 | 1.00 | 42.83 |
| 844 | NE2 | HIS | 108 | 26.820 | −1.531 | 4.613 | 1.00 | 42.83 |
| 845 | C | HIS | 108 | 26.567 | −0.048 | 0.136 | 1.00 | 52.57 |
| 846 | O | HIS | 108 | 25.398 | 0.192 | −0.174 | 1.00 | 52.57 |
| 847 | N | GLY | 109 | 27.144 | −1.226 | −0.072 | 1.00 | 46.92 |
| 848 | CA | GLY | 109 | 26.413 | −2.313 | −0.703 | 1.00 | 46.92 |
| 849 | C | GLY | 109 | 25.824 | −3.310 | 0.273 | 1.00 | 46.92 |
| 850 | O | GLY | 109 | 26.406 | −3.596 | 1.309 | 1.00 | 46.92 |
| 851 | N | TRP | 110 | 24.657 | −3.840 | −0.063 | 1.00 | 39.32 |
| 852 | CA | TRP | 110 | 23.983 | −4.832 | 0.771 | 1.00 | 39.32 |
| 853 | CB | TRP | 110 | 22.807 | −5.430 | −0.014 | 1.00 | 37.53 |
| 854 | CG | TRP | 110 | 22.002 | −6.428 | 0.744 | 1.00 | 37.53 |
| 855 | CD2 | TRP | 110 | 20.978 | −6.153 | 1.710 | 1.00 | 37.53 |
| 856 | CE2 | TRP | 110 | 20.507 | −7.399 | 2.183 | 1.00 | 37.53 |
| 857 | CE3 | TRP | 110 | 20.414 | −4.973 | 2.225 | 1.00 | 37.53 |
| 858 | CD1 | TRP | 110 | 22.105 | −7.785 | 0.672 | 1.00 | 37.53 |
| 859 | NE1 | TRP | 110 | 21.212 | −8.376 | 1.531 | 1.00 | 37.53 |
| 860 | CZ2 | TRP | 110 | 19.494 | −7.508 | 3.152 | 1.00 | 37.53 |
| 861 | CZ3 | TRP | 110 | 19.400 | −5.078 | 3.195 | 1.00 | 37.53 |
| 862 | CH2 | TRP | 110 | 18.954 | −6.344 | 3.645 | 1.00 | 37.53 |
| 863 | C | TRP | 110 | 24.991 | −5.918 | 1.134 | 1.00 | 39.32 |
| 864 | O | TRP | 110 | 25.816 | −6.297 | 0.309 | 1.00 | 39.32 |
| 865 | N | ARG | 111 | 24.938 | −6.405 | 2.369 | 1.00 | 45.97 |
| 866 | CA | ARG | 111 | 25.876 | −7.447 | 2.818 | 1.00 | 45.97 |
| 867 | CB | ARG | 111 | 25.607 | −8.754 | 2.093 | 1.00 | 58.00 |
| 868 | CG | ARG | 111 | 24.357 | −9.430 | 2.525 | 1.00 | 58.00 |
| 869 | CD | ARG | 111 | 24.273 | −10.832 | 1.934 | 1.00 | 58.00 |
| 870 | NE | ARG | 111 | 23.403 | −11.623 | 2.786 | 1.00 | 58.00 |
| 871 | CZ | ARG | 111 | 23.773 | −12.728 | 3.406 | 1.00 | 58.00 |
| 872 | NH1 | ARG | 111 | 25.005 | −13.203 | 3.260 | 1.00 | 58.00 |
| 873 | NH2 | ARG | 111 | 22.918 | −13.322 | 4.218 | 1.00 | 58.00 |
| 874 | C | ARG | 111 | 27.337 | −7.099 | 2.583 | 1.00 | 45.97 |
| 875 | O | ARG | 111 | 28.184 | −7.996 | 2.501 | 1.00 | 45.97 |
| 876 | N | ASN | 112 | 27.645 | −5.817 | 2.438 | 1.00 | 52.35 |
| 877 | CA | ASN | 112 | 29.017 | −5.420 | 2.172 | 1.00 | 52.35 |
| 878 | CB | ASN | 112 | 29.923 | −5.796 | 3.341 | 1.00 | 76.11 |
| 879 | CG | ASN | 112 | 29.974 | −4.718 | 4.374 | 1.00 | 76.11 |
| 880 | OD1 | ASN | 112 | 30.488 | −3.629 | 4.117 | 1.00 | 76.11 |
| 881 | ND2 | ASN | 112 | 29.427 | −4.997 | 5.553 | 1.00 | 76.11 |
| 882 | C | ASN | 112 | 29.581 | −6.010 | 0.883 | 1.00 | 52.35 |
| 883 | O | ASN | 112 | 30.778 | −6.304 | 0.792 | 1.00 | 52.35 |
| 884 | N | TRP | 113 | 28.719 | −6.180 | −0.110 | 1.00 | 53.59 |
| 885 | CA | TRP | 113 | 29.177 | −6.682 | −1.386 | 1.00 | 53.59 |
| 886 | CB | TRP | 113 | 27.991 | −6.922 | −2.333 | 1.00 | 53.83 |
| 887 | CG | TRP | 113 | 27.170 | −8.138 | −1.993 | 1.00 | 53.83 |
| 888 | CD2 | TRP | 113 | 25.778 | −8.348 | −2.273 | 1.00 | 53.83 |
| 889 | CE2 | TRP | 113 | 25.440 | −9.634 | −1.783 | 1.00 | 53.83 |
| 890 | CE3 | TRP | 113 | 24.779 | −7.578 | −2.895 | 1.00 | 53.83 |
| 891 | CD1 | TRP | 113 | 27.607 | −9.273 | −1.368 | 1.00 | 53.83 |
| 892 | NE1 | TRP | 113 | 26.578 | −10.173 | −1.236 | 1.00 | 53.83 |
| 893 | CZ2 | TRP | 113 | 24.144 | −10.169 | −1.888 | 1.00 | 53.83 |
| 894 | CZ3 | TRP | 113 | 23.490 | −8.107 | −3.002 | 1.00 | 53.83 |
| 895 | CH2 | TRP | 113 | 23.186 | −9.394 | −2.500 | 1.00 | 53.83 |
| 896 | C | TRP | 113 | 30.068 | −5.583 | −1.939 | 1.00 | 53.59 |
| 897 | O | TRP | 113 | 29.892 | −4.407 | −1.605 | 1.00 | 53.59 |
| 898 | N | ASP | 114 | 31.022 | −5.950 | −2.779 | 1.00 | 64.92 |
| 899 | CA | ASP | 114 | 31.893 | −4.950 | −3.363 | 1.00 | 64.92 |
| 900 | CB | ASP | 114 | 33.105 | −5.602 | −4.020 | 1.00 | 80.11 |
| 901 | CG | ASP | 114 | 33.906 | −6.427 | −3.046 | 1.00 | 80.11 |
| 902 | OD1 | ASP | 114 | 34.241 | −5.892 | −1.968 | 1.00 | 80.11 |
| 903 | OD2 | ASP | 114 | 34.195 | −7.603 | −3.355 | 1.00 | 80.11 |
| 904 | C | ASP | 114 | 31.122 | −4.148 | −4.390 | 1.00 | 64.92 |
| 905 | O | ASP | 114 | 30.332 | −4.691 | −5.170 | 1.00 | 64.92 |
| 906 | N | VAL | 115 | 31.343 | −2.844 | −4.375 | 1.00 | 69.99 |
| 907 | CA | VAL | 115 | 30.680 | −1.966 | −5.309 | 1.00 | 69.99 |
| 908 | CB | VAL | 115 | 29.938 | −0.865 | −4.556 | 1.00 | 39.79 |
| 909 | CG1 | VAL | 115 | 29.177 | 0.018 | −5.530 | 1.00 | 39.79 |
| 910 | CG2 | VAL | 115 | 28.996 | −1.485 | −3.555 | 1.00 | 39.79 |
| 911 | C | VAL | 115 | 31.764 | −1.376 | −6.198 | 1.00 | 69.99 |
| 912 | O | VAL | 115 | 32.797 | −0.930 | −5.701 | 1.00 | 69.99 |
| 913 | N | TYR | 116 | 31.540 | −1.392 | −7.511 | 1.00 | 52.13 |
| 914 | CA | TYR | 116 | 32.524 | −0.869 | −8.457 | 1.00 | 52.13 |
| 915 | CB | TYR | 116 | 33.016 | −1.988 | −9.377 | 1.00 | 78.35 |
| 916 | CG | TYR | 116 | 33.716 | −3.109 | −8.650 | 1.00 | 78.35 |
| 917 | CD1 | TYR | 116 | 33.009 | −4.214 | −8.178 | 1.00 | 78.35 |
| 918 | CE1 | TYR | 116 | 33.658 | −5.247 | −7.492 | 1.00 | 78.35 |
| 919 | CD2 | TYR | 116 | 35.093 | −3.057 | −8.419 | 1.00 | 78.35 |
| 920 | CE2 | TYR | 116 | 35.752 | −4.077 | −7.736 | 1.00 | 78.35 |
| 921 | CZ | TYR | 116 | 35.030 | −5.170 | −7.275 | 1.00 | 78.35 |
| 922 | OH | TYR | 116 | 35.684 | −6.180 | −6.600 | 1.00 | 78.35 |
| 923 | C | TYR | 116 | 31.946 | 0.258 | −9.292 | 1.00 | 52.13 |
| 924 | O | TYR | 116 | 30.749 | 0.507 | −9.242 | 1.00 | 52.13 |
| 925 | N | LYS | 117 | 32.795 | 0.933 | −10.063 | 1.00 | 52.62 |
| 926 | CA | LYS | 117 | 32.339 | 2.040 | −10.901 | 1.00 | 52.62 |
| 927 | CB | LYS | 117 | 31.535 | 1.523 | −12.102 | 1.00 | 75.37 |
| 928 | CG | LYS | 117 | 32.339 | 0.848 | −13.200 | 1.00 | 75.37 |
| 929 | CD | LYS | 117 | 31.480 | 0.605 | −14.444 | 1.00 | 75.37 |
| 930 | CE | LYS | 117 | 30.804 | 1.907 | −14.904 | 1.00 | 75.37 |
| 931 | NZ | LYS | 117 | 30.085 | 1.861 | −16.222 | 1.00 | 75.37 |
| 932 | C | LYS | 117 | 31.454 | 2.990 | −10.095 | 1.00 | 52.62 |
| 933 | O | LYS | 117 | 30.414 | 3.447 | −10.585 | 1.00 | 52.62 |
| 934 | N | VAL | 118 | 31.854 | 3.283 | −8.859 | 1.00 | 53.28 |
| 935 | CA | VAL | 118 | 31.052 | 4.171 | −8.029 | 1.00 | 53.28 |
| 936 | CB | VAL | 118 | 31.522 | 4.181 | −6.566 | 1.00 | 39.37 |
| 937 | CG1 | VAL | 118 | 30.807 | 5.300 | −5.793 | 1.00 | 39.37 |
| 938 | CG2 | VAL | 118 | 31.216 | 2.841 | −5.907 | 1.00 | 39.37 |
| 939 | C | VAL | 118 | 31.048 | 5.611 | −8.519 | 1.00 | 53.28 |
| 940 | O | VAL | 118 | 32.100 | 6.193 | −8.772 | 1.00 | 53.28 |
| 941 | N | ILE | 119 | 29.849 | 6.172 | −8.663 | 1.00 | 50.90 |
| 942 | CA | ILE | 119 | 29.704 | 7.555 | −9.072 | 1.00 | 50.90 |
| 943 | CB | ILE | 119 | 29.099 | 7.691 | −10.483 | 1.00 | 47.00 |
| 944 | CG2 | ILE | 119 | 28.899 | 9.175 | −10.821 | 1.00 | 47.00 |
| 945 | CG1 | ILE | 119 | 30.023 | 7.033 | −11.507 | 1.00 | 47.00 |
| 946 | CD1 | ILE | 119 | 29.506 | 7.082 | −12.911 | 1.00 | 47.00 |
| 947 | C | ILE | 119 | 28.760 | 8.211 | −8.090 | 1.00 | 50.90 |
| 948 | O | ILE | 119 | 27.767 | 7.606 | −7.703 | 1.00 | 50.90 |
| 949 | N | TYR | 120 | 29.087 | 9.429 | −7.661 | 1.00 | 46.78 |
| 950 | CA | TYR | 120 | 28.228 | 10.172 | −6.742 | 1.00 | 46.78 |
| 951 | CB | TYR | 120 | 29.018 | 10.751 | −5.568 | 1.00 | 47.71 |
| 952 | CG | TYR | 120 | 29.508 | 9.727 | −4.589 | 1.00 | 47.71 |
| 953 | CD1 | TYR | 120 | 30.764 | 9.150 | −4.734 | 1.00 | 47.71 |
| 954 | CE1 | TYR | 120 | 31.225 | 8.210 | −3.842 | 1.00 | 47.71 |
| 955 | CD2 | TYR | 120 | 28.716 | 9.332 | −3.518 | 1.00 | 47.71 |
| 956 | CE2 | TYR | 120 | 29.167 | 8.376 | −2.610 | 1.00 | 47.71 |
| 957 | CZ | TYR | 120 | 30.427 | 7.820 | −2.781 | 1.00 | 47.71 |
| 958 | OH | TYR | 120 | 30.894 | 6.859 | −1.911 | 1.00 | 47.71 |
| 959 | C | TYR | 120 | 27.559 | 11.319 | −7.483 | 1.00 | 46.78 |
| 960 | O | TYR | 120 | 28.166 | 11.972 | −8.330 | 1.00 | 46.78 |
| 961 | N | TYR | 121 | 26.306 | 11.572 | −7.152 | 1.00 | 39.53 |
| 962 | CA | TYR | 121 | 25.578 | 12.651 | −7.807 | 1.00 | 39.53 |
| 963 | CB | TYR | 121 | 24.378 | 12.092 | −8.584 | 1.00 | 51.28 |
| 964 | CG | TYR | 121 | 24.767 | 11.242 | −9.758 | 1.00 | 51.28 |
| 965 | CD1 | TYR | 121 | 25.075 | 9.897 | −9.598 | 1.00 | 51.28 |
| 966 | CE1 | TYR | 121 | 25.525 | 9.131 | −10.673 | 1.00 | 51.28 |
| 967 | CD2 | TYR | 121 | 24.909 | 11.802 | −11.025 | 1.00 | 51.28 |
| 968 | CE2 | TYR | 121 | 25.359 | 11.046 | −12.106 | 1.00 | 51.28 |
| 969 | CZ | TYR | 121 | 25.669 | 9.712 | −11.920 | 1.00 | 51.28 |
| 970 | OH | TYR | 121 | 26.158 | 8.978 | −12.979 | 1.00 | 51.28 |
| 971 | C | TYR | 121 | 25.079 | 13.703 | −6.834 | 1.00 | 39.53 |
| 972 | O | TYR | 121 | 24.692 | 13.392 | −5.711 | 1.00 | 39.53 |
| 973 | N | LYS | 122 | 25.104 | 14.955 | −7.263 | 1.00 | 48.43 |
| 974 | CA | LYS | 122 | 24.584 | 16.022 | −6.422 | 1.00 | 48.43 |
| 975 | CB | LYS | 122 | 25.704 | 16.886 | −5.851 | 1.00 | 57.69 |
| 976 | CG | LYS | 122 | 25.215 | 17.619 | −4.630 | 1.00 | 57.69 |
| 977 | CD | LYS | 122 | 25.990 | 18.882 | −4.345 | 1.00 | 57.69 |
| 978 | CE | LYS | 122 | 25.543 | 19.987 | −5.251 | 1.00 | 57.69 |
| 979 | NZ | LYS | 122 | 26.178 | 21.258 | −4.865 | 1.00 | 57.69 |
| 980 | C | LYS | 122 | 23.658 | 16.871 | −7.296 | 1.00 | 48.43 |
| 981 | O | LYS | 122 | 24.108 | 17.523 | −8.231 | 1.00 | 48.43 |
| 982 | N | ASP | 123 | 22.368 | 16.861 | −6.983 | 1.00 | 47.40 |
| 983 | CA | ASP | 123 | 21.384 | 17.609 | −7.783 | 1.00 | 47.40 |
| 984 | CB | ASP | 123 | 21.625 | 19.122 | −7.687 | 1.00 | 49.57 |
| 985 | CG | ASP | 123 | 21.713 | 19.611 | −6.269 | 1.00 | 49.57 |
| 986 | OD1 | ASP | 123 | 20.803 | 19.311 | −5.468 | 1.00 | 49.57 |
| 987 | OD2 | ASP | 123 | 22.699 | 20.301 | −5.953 | 1.00 | 49.57 |
| 988 | C | ASP | 123 | 21.565 | 17.197 | −9.241 | 1.00 | 47.40 |
| 989 | O | ASP | 123 | 21.856 | 18.044 | −10.095 | 1.00 | 47.40 |
| 990 | N | GLY | 124 | 21.433 | 15.904 | −9.521 | 1.00 | 52.66 |
| 991 | CA | GLY | 124 | 21.593 | 15.430 | −10.888 | 1.00 | 52.66 |
| 992 | C | GLY | 124 | 23.017 | 15.363 | −11.421 | 1.00 | 52.66 |
| 993 | O | GLY | 124 | 23.398 | 14.382 | −12.041 | 1.00 | 52.66 |
| 994 | N | GLU | 125 | 23.807 | 16.408 | −11.204 | 1.00 | 53.89 |
| 995 | CA | GLU | 125 | 25.198 | 16.447 | −11.672 | 1.00 | 53.89 |
| 996 | CB | GLU | 125 | 25.787 | 17.841 | −11.427 | 1.00 | 115.76 |
| 997 | CG | GLU | 125 | 25.113 | 18.952 | −12.208 | 1.00 | 115.76 |
| 998 | CD | GLU | 125 | 25.418 | 18.876 | −13.689 | 1.00 | 115.76 |
| 999 | OE1 | GLU | 125 | 26.615 | 18.910 | −14.044 | 1.00 | 115.76 |
| 1000 | OE2 | GLU | 125 | 24.469 | 18.784 | −14.498 | 1.00 | 115.76 |
| 1001 | C | GLU | 125 | 26.085 | 15.412 | −10.974 | 1.00 | 53.89 |
| 1002 | O | GLU | 125 | 25.912 | 15.130 | −9.790 | 1.00 | 53.89 |
| 1003 | N | ALA | 126 | 27.033 | 14.847 | −11.709 | 1.00 | 51.23 |
| 1004 | CA | ALA | 126 | 27.954 | 13.880 | −11.125 | 1.00 | 51.23 |
| 1005 | CB | ALA | 126 | 28.557 | 12.996 | −12.218 | 1.00 | 49.62 |
| 1006 | C | ALA | 126 | 29.051 | 14.673 | −10.422 | 1.00 | 51.23 |
| 1007 | O | ALA | 126 | 29.659 | 15.551 | −11.020 | 1.00 | 51.23 |
| 1008 | N | LEU | 127 | 29.302 | 14.373 | −9.157 | 1.00 | 63.08 |
| 1009 | CA | LEU | 127 | 30.332 | 15.088 | −8.409 | 1.00 | 63.08 |
| 1010 | CB | LEU | 127 | 30.641 | 14.370 | −7.104 | 1.00 | 50.82 |
| 1011 | CG | LEU | 127 | 29.663 | 14.719 | −6.003 | 1.00 | 50.82 |
| 1012 | CD1 | LEU | 127 | 30.265 | 14.281 | −4.689 | 1.00 | 50.82 |
| 1013 | CD2 | LEU | 127 | 29.403 | 16.221 | −6.004 | 1.00 | 50.82 |
| 1014 | C | LEU | 127 | 31.643 | 15.322 | −9.142 | 1.00 | 63.08 |
| 1015 | O | LEU | 127 | 32.233 | 14.384 | −9.688 | 1.00 | 63.08 |
| 1016 | N | LYS | 128 | 32.096 | 16.574 | −9.132 | 1.00 | 120.62 |
| 1017 | CA | LYS | 128 | 33.343 | 16.933 | −9.780 | 1.00 | 120.62 |
| 1018 | CB | LYS | 128 | 33.360 | 18.423 | −10.120 | 1.00 | 105.58 |
| 1019 | CG | LYS | 128 | 32.187 | 18.879 | −10.946 | 1.00 | 105.58 |
| 1020 | CD | LYS | 128 | 32.494 | 20.181 | −11.667 | 1.00 | 105.58 |
| 1021 | CE | LYS | 128 | 31.295 | 20.661 | −12.465 | 1.00 | 105.58 |
| 1022 | NZ | LYS | 128 | 30.167 | 21.011 | −11.563 | 1.00 | 105.58 |
| 1023 | C | LYS | 128 | 34.535 | 16.589 | −8.883 | 1.00 | 120.62 |
| 1024 | O | LYS | 128 | 35.604 | 16.232 | −9.381 | 1.00 | 120.62 |
| 1025 | N | TYR | 129 | 34.353 | 16.714 | −7.568 | 1.00 | 102.50 |
| 1026 | CA | TYR | 129 | 35.415 | 16.387 | −6.613 | 1.00 | 102.50 |
| 1027 | CB | TYR | 129 | 34.823 | 16.167 | −5.216 | 1.00 | 91.38 |
| 1028 | CG | TYR | 129 | 34.316 | 17.406 | −4.528 | 1.00 | 91.38 |
| 1029 | CD1 | TYR | 129 | 32.980 | 17.523 | −4.148 | 1.00 | 91.38 |
| 1030 | CE1 | TYR | 129 | 32.519 | 18.664 | −3.477 | 1.00 | 91.38 |
| 1031 | CD2 | TYR | 129 | 35.187 | 18.455 | −4.224 | 1.00 | 91.38 |
| 1032 | CE2 | TYR | 129 | 34.741 | 19.598 | −3.557 | 1.00 | 91.38 |
| 1033 | CZ | TYR | 129 | 33.408 | 19.699 | −3.185 | 1.00 | 91.38 |
| 1034 | OH | TYR | 129 | 32.960 | 20.831 | −2.533 | 1.00 | 91.38 |
| 1035 | C | TYR | 129 | 36.133 | 15.099 | −7.024 | 1.00 | 102.50 |
| 1036 | O | TYR | 129 | 35.553 | 14.244 | −7.692 | 1.00 | 102.50 |
| 1037 | N | TRP | 130 | 37.394 | 14.948 | −6.636 | 1.00 | 95.53 |
| 1038 | CA | TRP | 130 | 38.102 | 13.709 | −6.940 | 1.00 | 95.53 |
| 1039 | CB | TRP | 130 | 39.605 | 13.916 | −7.127 | 1.00 | 77.30 |
| 1040 | CG | TRP | 130 | 40.317 | 12.599 | −7.015 | 1.00 | 77.30 |
| 1041 | CD2 | TRP | 130 | 40.414 | 11.594 | −8.027 | 1.00 | 77.30 |
| 1042 | CE2 | TRP | 130 | 41.001 | 10.447 | −7.440 | 1.00 | 77.30 |
| 1043 | CE3 | TRP | 130 | 40.046 | 11.536 | −9.379 | 1.00 | 77.30 |
| 1044 | CD1 | TRP | 130 | 40.853 | 12.046 | −5.881 | 1.00 | 77.30 |
| 1045 | NE1 | TRP | 130 | 41.262 | 10.752 | −6.124 | 1.00 | 77.30 |
| 1046 | CZ2 | TRP | 130 | 41.229 | 9.269 | −8.149 | 1.00 | 77.30 |
| 1047 | CZ3 | TRP | 130 | 40.278 | 10.365 | −10.083 | 1.00 | 77.30 |
| 1048 | CH2 | TRP | 130 | 40.862 | 9.250 | −9.469 | 1.00 | 77.30 |
| 1049 | C | TRP | 130 | 37.903 | 12.742 | −5.784 | 1.00 | 95.53 |
| 1050 | O | TRP | 130 | 38.038 | 13.124 | −4.620 | 1.00 | 95.53 |
| 1051 | N | TYR | 131 | 37.589 | 11.488 | −6.091 | 1.00 | 71.08 |
| 1052 | CA | TYR | 131 | 37.397 | 10.505 | −5.034 | 1.00 | 71.08 |
| 1053 | CB | TYR | 131 | 35.934 | 10.495 | −4.565 | 1.00 | 66.47 |
| 1054 | CG | TYR | 131 | 34.894 | 10.405 | −5.657 | 1.00 | 66.47 |
| 1055 | CD1 | TYR | 131 | 34.370 | 9.178 | −6.055 | 1.00 | 66.47 |
| 1056 | CE1 | TYR | 131 | 33.355 | 9.111 | −7.009 | 1.00 | 66.47 |
| 1057 | CD2 | TYR | 131 | 34.387 | 11.562 | −6.246 | 1.00 | 66.47 |
| 1058 | CE2 | TYR | 131 | 33.375 | 11.506 | −7.201 | 1.00 | 66.47 |
| 1059 | CZ | TYR | 131 | 32.857 | 10.283 | −7.572 | 1.00 | 66.47 |
| 1060 | OH | TYR | 131 | 31.819 | 10.246 | −8.477 | 1.00 | 66.47 |
| 1061 | C | TYR | 131 | 37.838 | 9.098 | −5.384 | 1.00 | 71.08 |
| 1062 | O | TYR | 131 | 38.058 | 8.768 | −6.554 | 1.00 | 71.08 |
| 1063 | N | GLU | 132 | 37.970 | 8.277 | −4.345 | 1.00 | 130.21 |
| 1064 | CA | GLU | 132 | 38.389 | 6.884 | −4.467 | 1.00 | 130.21 |
| 1065 | CB | GLU | 132 | 39.252 | 6.520 | −3.266 | 1.00 | 141.76 |
| 1066 | CG | GLU | 132 | 38.484 | 6.649 | −1.959 | 1.00 | 141.76 |
| 1067 | CD | GLU | 132 | 39.311 | 7.221 | −0.824 | 1.00 | 141.76 |
| 1068 | OE1 | GLU | 132 | 40.544 | 7.379 | −0.985 | 1.00 | 141.76 |
| 1069 | OE2 | GLU | 132 | 38.717 | 7.507 | 0.239 | 1.00 | 141.76 |
| 1070 | C | GLU | 132 | 37.135 | 6.013 | −4.479 | 1.00 | 130.21 |
| 1071 | O | GLU | 132 | 36.031 | 6.503 | −4.234 | 1.00 | 130.21 |
| 1072 | N | ASN | 133 | 37.307 | 4.724 | −4.745 | 1.00 | 122.18 |
| 1073 | CA | ASN | 133 | 36.182 | 3.803 | −4.794 | 1.00 | 122.18 |
| 1074 | CB | ASN | 133 | 36.698 | 2.392 | −5.090 | 1.00 | 141.76 |
| 1075 | CG | ASN | 133 | 35.592 | 1.428 | −5.502 | 1.00 | 141.76 |
| 1076 | OD1 | ASN | 133 | 34.412 | 1.777 | −5.522 | 1.00 | 141.76 |
| 1077 | ND2 | ASN | 133 | 35.980 | 0.200 | −5.831 | 1.00 | 141.76 |
| 1078 | C | ASN | 133 | 35.348 | 3.806 | −3.505 | 1.00 | 122.18 |
| 1079 | O | ASN | 133 | 35.879 | 3.627 | −2.406 | 1.00 | 122.18 |
| 1080 | N | HIS | 134 | 34.044 | 4.032 | −3.681 | 1.00 | 110.94 |
| 1081 | CA | HIS | 134 | 33.013 | 4.052 | −2.630 | 1.00 | 110.94 |
| 1082 | CB | HIS | 134 | 32.503 | 2.627 | −2.355 | 1.00 | 116.53 |
| 1083 | CG | HIS | 134 | 33.300 | 1.858 | −1.341 | 1.00 | 116.53 |
| 1084 | CD2 | HIS | 134 | 33.153 | 1.742 | −0.001 | 1.00 | 116.53 |
| 1085 | ND1 | HIS | 134 | 34.341 | 1.029 | −1.687 | 1.00 | 116.53 |
| 1086 | CE1 | HIS | 134 | 34.797 | 0.425 | −0.605 | 1.00 | 116.53 |
| 1087 | NE2 | HIS | 134 | 34.091 | 0.837 | 0.431 | 1.00 | 116.53 |
| 1088 | C | HIS | 134 | 33.169 | 4.773 | −1.283 | 1.00 | 110.94 |
| 1089 | O | HIS | 134 | 32.312 | 4.627 | −0.408 | 1.00 | 110.94 |
| 1090 | N | ASN | 135 | 34.245 | 5.532 | −1.105 | 1.00 | 107.95 |
| 1091 | CA | ASN | 135 | 34.430 | 6.296 | 0.125 | 1.00 | 107.95 |
| 1092 | CB | ASN | 135 | 35.464 | 5.654 | 1.057 | 1.00 | 141.21 |
| 1093 | CG | ASN | 135 | 35.389 | 6.207 | 2.490 | 1.00 | 141.21 |
| 1094 | OD1 | ASN | 135 | 34.536 | 7.044 | 2.801 | 1.00 | 141.21 |
| 1095 | ND2 | ASN | 135 | 36.271 | 5.735 | 3.358 | 1.00 | 141.21 |
| 1096 | C | ASN | 135 | 34.900 | 7.680 | −0.310 | 1.00 | 107.95 |
| 1097 | O | ASN | 135 | 35.952 | 7.836 | −0.925 | 1.00 | 107.95 |
| 1098 | N | ILE | 136 | 34.095 | 8.685 | −0.005 | 1.00 | 65.75 |
| 1099 | CA | ILE | 136 | 34.421 | 10.048 | −0.387 | 1.00 | 65.75 |
| 1100 | CB | ILE | 136 | 33.401 | 10.550 | −1.433 | 1.00 | 65.18 |
| 1101 | CG2 | ILE | 136 | 32.002 | 10.306 | −0.943 | 1.00 | 65.18 |
| 1102 | CG1 | ILE | 136 | 33.587 | 12.030 | −1.717 | 1.00 | 65.18 |
| 1103 | CD1 | ILE | 136 | 32.601 | 12.535 | −2.780 | 1.00 | 65.18 |
| 1104 | C | ILE | 136 | 34.457 | 10.983 | 0.826 | 1.00 | 65.75 |
| 1105 | O | ILE | 136 | 33.593 | 10.924 | 1.706 | 1.00 | 65.75 |
| 1106 | N | SER | 137 | 35.475 | 11.838 | 0.863 | 1.00 | 51.67 |
| 1107 | CA | SER | 137 | 35.646 | 12.785 | 1.957 | 1.00 | 51.67 |
| 1108 | CB | SER | 137 | 36.944 | 12.470 | 2.714 | 1.00 | 87.54 |
| 1109 | OG | SER | 137 | 37.068 | 13.249 | 3.889 | 1.00 | 87.54 |
| 1110 | C | SER | 137 | 35.678 | 14.225 | 1.449 | 1.00 | 51.67 |
| 1111 | O | SER | 137 | 36.522 | 14.593 | 0.629 | 1.00 | 51.67 |
| 1112 | N | ILE | 138 | 34.729 | 15.021 | 1.924 | 1.00 | 63.18 |
| 1113 | CA | ILE | 138 | 34.634 | 16.431 | 1.559 | 1.00 | 63.18 |
| 1114 | CB | ILE | 138 | 33.178 | 16.801 | 1.239 | 1.00 | 64.58 |
| 1115 | CG2 | ILE | 138 | 33.030 | 18.301 | 1.103 | 1.00 | 64.58 |
| 1116 | CG1 | ILE | 138 | 32.754 | 16.080 | −0.043 | 1.00 | 64.58 |
| 1117 | CD1 | ILE | 138 | 31.285 | 16.134 | −0.328 | 1.00 | 64.58 |
| 1118 | C | ILE | 138 | 35.119 | 17.126 | 2.815 | 1.00 | 63.18 |
| 1119 | O | ILE | 138 | 34.379 | 17.271 | 3.786 | 1.00 | 63.18 |
| 1120 | N | THR | 139 | 36.384 | 17.525 | 2.783 | 1.00 | 80.92 |
| 1121 | CA | THR | 139 | 37.063 | 18.140 | 3.920 | 1.00 | 80.92 |
| 1122 | CB | THR | 139 | 38.557 | 18.282 | 3.609 | 1.00 | 76.51 |
| 1123 | OG1 | THR | 139 | 38.721 | 19.040 | 2.403 | 1.00 | 76.51 |
| 1124 | CG2 | THR | 139 | 39.189 | 16.909 | 3.432 | 1.00 | 76.51 |
| 1125 | C | THR | 139 | 36.576 | 19.468 | 4.498 | 1.00 | 80.92 |
| 1126 | O | THR | 139 | 36.484 | 19.612 | 5.721 | 1.00 | 80.92 |
| 1127 | N | ASN | 140 | 36.277 | 20.441 | 3.644 | 1.00 | 57.80 |
| 1128 | CA | ASN | 140 | 35.837 | 21.738 | 4.151 | 1.00 | 57.80 |
| 1129 | CB | ASN | 140 | 36.840 | 22.821 | 3.731 | 1.00 | 95.08 |
| 1130 | CG | ASN | 140 | 37.066 | 23.863 | 4.812 | 1.00 | 95.08 |
| 1131 | OD1 | ASN | 140 | 36.122 | 24.315 | 5.458 | 1.00 | 95.08 |
| 1132 | ND2 | ASN | 140 | 38.320 | 24.259 | 5.005 | 1.00 | 95.08 |
| 1133 | C | ASN | 140 | 34.438 | 22.108 | 3.657 | 1.00 | 57.80 |
| 1134 | O | ASN | 140 | 34.300 | 22.924 | 2.752 | 1.00 | 57.80 |
| 1135 | N | ALA | 141 | 33.413 | 21.512 | 4.265 | 1.00 | 63.45 |
| 1136 | CA | ALA | 141 | 32.031 | 21.765 | 3.871 | 1.00 | 63.45 |
| 1137 | CB | ALA | 141 | 31.060 | 21.206 | 4.914 | 1.00 | 40.09 |
| 1138 | C | ALA | 141 | 31.740 | 23.242 | 3.642 | 1.00 | 63.45 |
| 1139 | O | ALA | 141 | 32.185 | 24.106 | 4.399 | 1.00 | 63.45 |
| 1140 | N | THR | 142 | 30.971 | 23.505 | 2.588 | 1.00 | 60.40 |
| 1141 | CA | THR | 142 | 30.573 | 24.847 | 2.192 | 1.00 | 60.40 |
| 1142 | CB | THR | 142 | 31.247 | 25.210 | 0.871 | 1.00 | 65.36 |
| 1143 | OG1 | THR | 142 | 32.580 | 25.646 | 1.137 | 1.00 | 65.36 |
| 1144 | CG2 | THR | 142 | 30.489 | 26.292 | 0.144 | 1.00 | 65.36 |
| 1145 | C | THR | 142 | 29.062 | 24.871 | 2.017 | 1.00 | 60.40 |
| 1146 | O | THR | 142 | 28.432 | 23.815 | 1.955 | 1.00 | 60.40 |
| 1147 | N | VAL | 143 | 28.471 | 26.061 | 1.956 | 1.00 | 51.83 |
| 1148 | CA | VAL | 143 | 27.031 | 26.150 | 1.762 | 1.00 | 51.83 |
| 1149 | CB | VAL | 143 | 26.547 | 27.626 | 1.744 | 1.00 | 51.35 |
| 1150 | CG1 | VAL | 143 | 27.058 | 28.335 | 0.507 | 1.00 | 51.35 |
| 1151 | CG2 | VAL | 143 | 25.019 | 27.673 | 1.807 | 1.00 | 51.35 |
| 1152 | C | VAL | 143 | 26.667 | 25.448 | 0.434 | 1.00 | 51.83 |
| 1153 | O | VAL | 143 | 25.584 | 24.884 | 0.292 | 1.00 | 51.83 |
| 1154 | N | GLU | 144 | 27.599 | 25.466 | −0.513 | 1.00 | 54.88 |
| 1155 | CA | GLU | 144 | 27.411 | 24.836 | −1.812 | 1.00 | 54.88 |
| 1156 | CB | GLU | 144 | 28.554 | 25.200 | −2.749 | 1.00 | 66.83 |
| 1157 | CG | GLU | 144 | 28.639 | 26.639 | −3.146 | 1.00 | 66.83 |
| 1158 | CD | GLU | 144 | 29.941 | 26.929 | −3.860 | 1.00 | 66.83 |
| 1159 | OE1 | GLU | 144 | 30.280 | 26.169 | −4.799 | 1.00 | 66.83 |
| 1160 | OE2 | GLU | 144 | 30.624 | 27.908 | −3.478 | 1.00 | 66.83 |
| 1161 | C | GLU | 144 | 27.348 | 23.308 | −1.754 | 1.00 | 54.88 |
| 1162 | O | GLU | 144 | 26.862 | 22.675 | −2.685 | 1.00 | 54.88 |
| 1163 | N | ASP | 145 | 27.869 | 22.707 | −0.692 | 1.00 | 47.71 |
| 1164 | CA | ASP | 145 | 27.840 | 21.258 | −0.607 | 1.00 | 47.71 |
| 1165 | CB | ASP | 145 | 28.902 | 20.746 | 0.360 | 1.00 | 46.72 |
| 1166 | CG | ASP | 145 | 30.292 | 21.045 | −0.116 | 1.00 | 46.72 |
| 1167 | OD1 | ASP | 145 | 30.613 | 20.700 | −1.268 | 1.00 | 46.72 |
| 1168 | OD2 | ASP | 145 | 31.077 | 21.629 | 0.667 | 1.00 | 46.72 |
| 1169 | C | ASP | 145 | 26.463 | 20.785 | −0.200 | 1.00 | 47.71 |
| 1170 | O | ASP | 145 | 26.227 | 19.592 | −0.055 | 1.00 | 47.71 |
| 1171 | N | SER | 146 | 25.549 | 21.727 | −0.007 | 1.00 | 42.81 |
| 1172 | CA | SER | 146 | 24.175 | 21.355 | 0.314 | 1.00 | 42.81 |
| 1173 | CB | SER | 146 | 23.363 | 22.573 | 0.750 | 1.00 | 49.76 |
| 1174 | OG | SER | 146 | 23.841 | 23.139 | 1.955 | 1.00 | 49.76 |
| 1175 | C | SER | 146 | 23.583 | 20.794 | −0.993 | 1.00 | 42.81 |
| 1176 | O | SER | 146 | 24.014 | 21.160 | −2.091 | 1.00 | 42.81 |
| 1177 | N | GLY | 147 | 22.611 | 19.905 | −0.879 | 1.00 | 56.97 |
| 1178 | CA | GLY | 147 | 22.008 | 19.334 | −2.064 | 1.00 | 56.97 |
| 1179 | C | GLY | 147 | 21.419 | 17.975 | −1.766 | 1.00 | 56.97 |
| 1180 | O | GLY | 147 | 21.382 | 17.546 | −0.612 | 1.00 | 56.97 |
| 1181 | N | THR | 148 | 20.937 | 17.294 | −2.797 | 1.00 | 35.67 |
| 1182 | CA | THR | 148 | 20.371 | 15.968 | −2.594 | 1.00 | 35.67 |
| 1183 | CB | THR | 148 | 18.945 | 15.784 | −3.251 | 1.00 | 41.21 |
| 1184 | OG1 | THR | 148 | 19.077 | 15.127 | −4.522 | 1.00 | 41.21 |
| 1185 | CG2 | THR | 148 | 18.251 | 17.118 | −3.427 | 1.00 | 41.21 |
| 1186 | C | THR | 148 | 21.386 | 15.106 | −3.297 | 1.00 | 35.67 |
| 1187 | O | THR | 148 | 21.853 | 15.447 | −4.382 | 1.00 | 35.67 |
| 1188 | N | TYR | 149 | 21.743 | 13.998 | −2.668 | 1.00 | 37.71 |
| 1189 | CA | TYR | 149 | 22.753 | 13.118 | −3.233 | 1.00 | 37.71 |
| 1190 | CB | TYR | 149 | 23.988 | 13.045 | −2.317 | 1.00 | 40.71 |
| 1191 | CG | TYR | 149 | 24.803 | 14.300 | −2.117 | 1.00 | 40.71 |
| 1192 | CD1 | TYR | 149 | 24.289 | 15.396 | −1.442 | 1.00 | 40.71 |
| 1193 | CE1 | TYR | 149 | 25.081 | 16.544 | −1.214 | 1.00 | 40.71 |
| 1194 | CD2 | TYR | 149 | 26.117 | 14.366 | −2.568 | 1.00 | 40.71 |
| 1195 | CE2 | TYR | 149 | 26.907 | 15.494 | −2.357 | 1.00 | 40.71 |
| 1196 | CZ | TYR | 149 | 26.389 | 16.581 | −1.680 | 1.00 | 40.71 |
| 1197 | OH | TYR | 149 | 27.168 | 17.712 | −1.506 | 1.00 | 40.71 |
| 1198 | C | TYR | 149 | 22.234 | 11.701 | −3.349 | 1.00 | 37.71 |
| 1199 | O | TYR | 149 | 21.256 | 11.316 | −2.690 | 1.00 | 37.71 |
| 1200 | N | TYR | 150 | 22.918 | 10.930 | −4.183 | 1.00 | 32.88 |
| 1201 | CA | TYR | 150 | 22.629 | 9.526 | −4.333 | 1.00 | 32.88 |
| 1202 | CB | TYR | 150 | 21.325 | 9.288 | −5.123 | 1.00 | 48.85 |
| 1203 | CG | TYR | 150 | 21.384 | 9.537 | −6.605 | 1.00 | 48.85 |
| 1204 | CD1 | TYR | 150 | 21.813 | 8.534 | −7.483 | 1.00 | 48.85 |
| 1205 | CE1 | TYR | 150 | 21.854 | 8.748 | −8.853 | 1.00 | 48.85 |
| 1206 | CD2 | TYR | 150 | 20.997 | 10.765 | −7.138 | 1.00 | 48.85 |
| 1207 | CE2 | TYR | 150 | 21.038 | 10.990 | −8.502 | 1.00 | 48.85 |
| 1208 | CZ | TYR | 150 | 21.470 | 9.979 | −9.353 | 1.00 | 48.85 |
| 1209 | OH | TYR | 150 | 21.566 | 10.223 | −10.700 | 1.00 | 48.85 |
| 1210 | C | TYR | 150 | 23.853 | 9.009 | −5.047 | 1.00 | 32.88 |
| 1211 | O | TYR | 150 | 24.668 | 9.802 | −5.513 | 1.00 | 32.88 |
| 1212 | N | CYS | 151 | 24.018 | 7.699 | −5.108 | 1.00 | 43.72 |
| 1213 | CA | CYS | 151 | 25.173 | 7.151 | −5.793 | 1.00 | 43.72 |
| 1214 | C | CYS | 151 | 24.734 | 5.984 | −6.660 | 1.00 | 43.72 |
| 1215 | O | CYS | 151 | 23.634 | 5.457 | −6.488 | 1.00 | 43.72 |
| 1216 | CB | CYS | 151 | 26.229 | 6.672 | −4.779 | 1.00 | 45.51 |
| 1217 | SG | CYS | 151 | 25.716 | 5.340 | −3.631 | 1.00 | 45.51 |
| 1218 | N | THR | 152 | 25.605 | 5.601 | −7.588 | 1.00 | 49.22 |
| 1219 | CA | THR | 152 | 25.366 | 4.470 | −8.467 | 1.00 | 49.22 |
| 1220 | CB | THR | 152 | 25.033 | 4.892 | −9.940 | 1.00 | 42.84 |
| 1221 | OG1 | THR | 152 | 26.153 | 5.566 | −10.535 | 1.00 | 42.84 |
| 1222 | CG2 | THR | 152 | 23.823 | 5.794 | −9.968 | 1.00 | 42.84 |
| 1223 | C | THR | 152 | 26.647 | 3.655 | −8.467 | 1.00 | 49.22 |
| 1224 | O | THR | 152 | 27.752 | 4.192 | −8.290 | 1.00 | 49.22 |
| 1225 | N | GLY | 153 | 26.501 | 2.352 | −8.638 | 1.00 | 49.54 |
| 1226 | CA | GLY | 153 | 27.664 | 1.499 | −8.669 | 1.00 | 49.54 |
| 1227 | C | GLY | 153 | 27.298 | 0.134 | −9.203 | 1.00 | 49.54 |
| 1228 | O | GLY | 153 | 26.118 | −0.243 | −9.243 | 1.00 | 49.54 |
| 1229 | N | LYS | 154 | 28.314 | −0.610 | −9.618 | 1.00 | 50.10 |
| 1230 | CA | LYS | 154 | 28.108 | −1.946 | −10.141 | 1.00 | 50.10 |
| 1231 | CB | LYS | 154 | 29.078 | −2.209 | −11.297 | 1.00 | 64.36 |
| 1232 | CG | LYS | 154 | 28.956 | −3.599 | −11.885 | 1.00 | 64.36 |
| 1233 | CD | LYS | 154 | 30.078 | −3.925 | −12.851 | 1.00 | 64.36 |
| 1234 | CE | LYS | 154 | 30.006 | −5.382 | −13.286 | 1.00 | 64.36 |
| 1235 | NZ | LYS | 154 | 31.019 | −5.728 | −14.324 | 1.00 | 64.36 |
| 1236 | C | LYS | 154 | 28.310 | −2.994 | −9.048 | 1.00 | 50.10 |
| 1237 | O | LYS | 154 | 29.402 | −3.123 | −8.497 | 1.00 | 50.10 |
| 1238 | N | VAL | 155 | 27.236 | −3.709 | −8.714 | 1.00 | 62.56 |
| 1239 | CA | VAL | 155 | 27.295 | −4.784 | −7.727 | 1.00 | 62.56 |
| 1240 | CB | VAL | 155 | 26.139 | −4.722 | −6.732 | 1.00 | 41.01 |
| 1241 | CG1 | VAL | 155 | 26.266 | −5.872 | −5.742 | 1.00 | 41.01 |
| 1242 | CG2 | VAL | 155 | 26.132 | −3.391 | −6.009 | 1.00 | 41.01 |
| 1243 | C | VAL | 155 | 27.163 | −6.063 | −8.546 | 1.00 | 62.56 |
| 1244 | O | VAL | 155 | 26.211 | −6.224 | −9.312 | 1.00 | 62.56 |
| 1245 | N | TRP | 156 | 28.110 | −6.975 | −8.380 | 1.00 | 74.40 |
| 1246 | CA | TRP | 156 | 28.104 | −8.205 | −9.159 | 1.00 | 74.40 |
| 1247 | CB | TRP | 156 | 26.846 | −9.049 | −8.887 | 1.00 | 64.29 |
| 1248 | CG | TRP | 156 | 26.728 | −9.536 | −7.464 | 1.00 | 64.29 |
| 1249 | CD2 | TRP | 156 | 27.569 | −10.497 | −6.808 | 1.00 | 64.29 |
| 1250 | CE2 | TRP | 156 | 27.114 | −10.605 | −5.472 | 1.00 | 64.29 |
| 1251 | CE3 | TRP | 156 | 28.661 | −11.276 | −7.218 | 1.00 | 64.29 |
| 1252 | CD1 | TRP | 156 | 25.826 | −9.119 | −6.528 | 1.00 | 64.29 |
| 1253 | NE1 | TRP | 156 | 26.052 | −9.753 | −5.330 | 1.00 | 64.29 |
| 1254 | CZ2 | TRP | 156 | 27.717 | −11.464 | −4.537 | 1.00 | 64.29 |
| 1255 | CZ3 | TRP | 156 | 29.260 | −12.130 | −6.290 | 1.00 | 64.29 |
| 1256 | CH2 | TRP | 156 | 28.783 | −12.215 | −4.961 | 1.00 | 64.29 |
| 1257 | C | TRP | 156 | 28.162 | −7.814 | −10.639 | 1.00 | 74.40 |
| 1258 | O | TRP | 156 | 29.121 | −7.178 | −11.088 | 1.00 | 74.40 |
| 1259 | N | GLN | 157 | 27.128 | −8.163 | −11.397 | 1.00 | 71.89 |
| 1260 | CA | GLN | 157 | 27.132 | −7.841 | −12.823 | 1.00 | 71.89 |
| 1261 | CB | GLN | 157 | 26.876 | −9.105 | −13.650 | 1.00 | 111.42 |
| 1262 | CG | GLN | 157 | 28.041 | −10.072 | −13.672 | 1.00 | 111.42 |
| 1263 | CD | GLN | 157 | 29.351 | −9.380 | −13.965 | 1.00 | 111.42 |
| 1264 | OE1 | GLN | 157 | 29.504 | −8.683 | −14.969 | 1.00 | 111.42 |
| 1265 | NE2 | GLN | 157 | 30.312 | −9.570 | −13.081 | 1.00 | 111.42 |
| 1266 | C | GLN | 157 | 26.192 | −6.731 | −13.292 | 1.00 | 71.89 |
| 1267 | O | GLN | 157 | 26.153 | −6.418 | −14.476 | 1.00 | 71.89 |
| 1268 | N | LEU | 158 | 25.437 | −6.124 | −12.385 | 1.00 | 61.40 |
| 1269 | CA | LEU | 158 | 24.522 | −5.061 | −12.789 | 1.00 | 61.40 |
| 1270 | CB | LEU | 158 | 23.078 | −5.463 | −12.469 | 1.00 | 60.41 |
| 1271 | CG | LEU | 158 | 22.575 | −6.756 | −13.113 | 1.00 | 60.41 |
| 1272 | CD1 | LEU | 158 | 21.104 | −6.948 | −12.777 | 1.00 | 60.41 |
| 1273 | CD2 | LEU | 158 | 22.778 | −6.677 | −14.605 | 1.00 | 60.41 |
| 1274 | C | LEU | 158 | 24.822 | −3.703 | −12.149 | 1.00 | 61.40 |
| 1275 | O | LEU | 158 | 25.619 | −3.596 | −11.213 | 1.00 | 61.40 |
| 1276 | N | ASP | 159 | 24.167 | −2.668 | −12.662 | 1.00 | 56.42 |
| 1277 | CA | ASP | 159 | 24.338 | −1.322 | −12.140 | 1.00 | 56.42 |
| 1278 | CB | ASP | 159 | 24.465 | −0.311 | −13.276 | 1.00 | 74.24 |
| 1279 | CG | ASP | 159 | 25.653 | −0.586 | −14.170 | 1.00 | 74.24 |
| 1280 | OD1 | ASP | 159 | 26.794 | −0.666 | −13.663 | 1.00 | 74.24 |
| 1281 | OD2 | ASP | 159 | 25.444 | −0.721 | −15.392 | 1.00 | 74.24 |
| 1282 | C | ASP | 159 | 23.135 | −0.972 | −11.282 | 1.00 | 56.42 |
| 1283 | O | ASP | 159 | 21.992 | −1.211 | −11.680 | 1.00 | 56.42 |
| 1284 | N | TYR | 160 | 23.390 | −0.419 | −10.098 | 1.00 | 43.45 |
| 1285 | CA | TYR | 160 | 22.303 | −0.038 | −9.214 | 1.00 | 43.45 |
| 1286 | CB | TYR | 160 | 22.309 | −0.884 | −7.936 | 1.00 | 50.12 |
| 1287 | CG | TYR | 160 | 22.158 | −2.369 | −8.182 | 1.00 | 50.12 |
| 1288 | CD1 | TYR | 160 | 23.210 | −3.115 | −8.705 | 1.00 | 50.12 |
| 1289 | CE1 | TYR | 160 | 23.076 | −4.483 | −8.933 | 1.00 | 50.12 |
| 1290 | CD2 | TYR | 160 | 20.961 | −3.026 | −7.896 | 1.00 | 50.12 |
| 1291 | CE2 | TYR | 160 | 20.814 | −4.392 | −8.121 | 1.00 | 50.12 |
| 1292 | CZ | TYR | 160 | 21.875 | −5.113 | −8.637 | 1.00 | 50.12 |
| 1293 | OH | TYR | 160 | 21.760 | −6.467 | −8.840 | 1.00 | 50.12 |
| 1294 | C | TYR | 160 | 22.384 | 1.437 | −8.868 | 1.00 | 43.45 |
| 1295 | O | TYR | 160 | 23.341 | 2.125 | −9.219 | 1.00 | 43.45 |
| 1296 | N | GLU | 161 | 21.370 | 1.922 | −8.175 | 1.00 | 46.75 |
| 1297 | CA | GLU | 161 | 21.304 | 3.318 | −7.810 | 1.00 | 46.75 |
| 1298 | CB | GLU | 161 | 20.454 | 4.057 | −8.847 | 1.00 | 65.60 |
| 1299 | CG | GLU | 161 | 19.930 | 5.421 | −8.439 | 1.00 | 65.60 |
| 1300 | CD | GLU | 161 | 19.318 | 6.190 | −9.614 | 1.00 | 65.60 |
| 1301 | OE1 | GLU | 161 | 18.653 | 7.226 | −9.377 | 1.00 | 65.60 |
| 1302 | OE2 | GLU | 161 | 19.517 | 5.765 | −10.777 | 1.00 | 65.60 |
| 1303 | C | GLU | 161 | 20.687 | 3.385 | −6.432 | 1.00 | 46.75 |
| 1304 | O | GLU | 161 | 19.707 | 2.702 | −6.148 | 1.00 | 46.75 |
| 1305 | N | SER | 162 | 21.270 | 4.190 | −5.559 | 1.00 | 41.55 |
| 1306 | CA | SER | 162 | 20.743 | 4.297 | −4.202 | 1.00 | 41.55 |
| 1307 | CB | SER | 162 | 21.841 | 4.761 | −3.241 | 1.00 | 41.77 |
| 1308 | OG | SER | 162 | 22.155 | 6.124 | −3.467 | 1.00 | 41.77 |
| 1309 | C | SER | 162 | 19.622 | 5.311 | −4.170 | 1.00 | 41.55 |
| 1310 | O | SER | 162 | 19.458 | 6.079 | −5.103 | 1.00 | 41.55 |
| 1311 | N | GLU | 163 | 18.856 | 5.294 | −3.088 | 1.00 | 45.18 |
| 1312 | CA | GLU | 163 | 17.794 | 6.261 | −2.881 | 1.00 | 45.18 |
| 1313 | CB | GLU | 163 | 16.998 | 5.907 | −1.632 | 1.00 | 80.94 |
| 1314 | CG | GLU | 163 | 16.137 | 4.687 | −1.787 | 1.00 | 80.94 |
| 1315 | CD | GLU | 163 | 14.993 | 4.941 | −2.730 | 1.00 | 80.94 |
| 1316 | OE1 | GLU | 163 | 14.163 | 5.812 | −2.407 | 1.00 | 80.94 |
| 1317 | OE2 | GLU | 163 | 14.922 | 4.285 | −3.794 | 1.00 | 80.94 |
| 1318 | C | GLU | 163 | 18.535 | 7.576 | −2.653 | 1.00 | 45.18 |
| 1319 | O | GLU | 163 | 19.687 | 7.581 | −2.236 | 1.00 | 45.18 |
| 1320 | N | PRO | 164 | 17.893 | 8.709 | −2.928 | 1.00 | 47.28 |
| 1321 | CD | PRO | 164 | 16.592 | 8.936 | −3.576 | 1.00 | 31.71 |
| 1322 | CA | PRO | 164 | 18.598 | 9.970 | −2.712 | 1.00 | 47.28 |
| 1323 | CB | PRO | 164 | 17.888 | 10.913 | −3.677 | 1.00 | 31.71 |
| 1324 | CG | PRO | 164 | 16.472 | 10.450 | −3.551 | 1.00 | 31.71 |
| 1325 | C | PRO | 164 | 18.525 | 10.444 | −1.256 | 1.00 | 47.28 |
| 1326 | O | PRO | 164 | 17.624 | 10.084 | −0.497 | 1.00 | 47.28 |
| 1327 | N | LEU | 165 | 19.480 | 11.263 | −0.868 | 1.00 | 31.92 |
| 1328 | CA | LEU | 165 | 19.513 | 11.769 | 0.499 | 1.00 | 31.92 |
| 1329 | CB | LEU | 165 | 20.705 | 11.139 | 1.228 | 1.00 | 52.60 |
| 1330 | CG | LEU | 165 | 21.098 | 11.756 | 2.561 | 1.00 | 52.60 |
| 1331 | CD1 | LEU | 165 | 19.980 | 11.527 | 3.565 | 1.00 | 52.60 |
| 1332 | CD2 | LEU | 165 | 22.404 | 11.139 | 3.029 | 1.00 | 52.60 |
| 1333 | C | LEU | 165 | 19.697 | 13.275 | 0.454 | 1.00 | 31.92 |
| 1334 | O | LEU | 165 | 20.401 | 13.775 | −0.416 | 1.00 | 31.92 |
| 1335 | N | ASN | 166 | 19.084 | 14.007 | 1.366 | 1.00 | 38.25 |
| 1336 | CA | ASN | 166 | 19.297 | 15.450 | 1.345 | 1.00 | 38.25 |
| 1337 | CB | ASN | 166 | 17.969 | 16.186 | 1.543 | 1.00 | 38.79 |
| 1338 | CG | ASN | 166 | 17.056 | 16.079 | 0.309 | 1.00 | 38.79 |
| 1339 | OD1 | ASN | 166 | 17.546 | 15.847 | −0.801 | 1.00 | 38.79 |
| 1340 | ND2 | ASN | 166 | 15.748 | 16.259 | 0.490 | 1.00 | 38.79 |
| 1341 | C | ASN | 166 | 20.341 | 15.889 | 2.398 | 1.00 | 38.25 |
| 1342 | O | ASN | 166 | 20.282 | 15.480 | 3.561 | 1.00 | 38.25 |
| 1343 | N | ILE | 167 | 21.309 | 16.695 | 1.978 | 1.00 | 41.84 |
| 1344 | CA | ILE | 167 | 22.326 | 17.187 | 2.894 | 1.00 | 41.84 |
| 1345 | CB | ILE | 167 | 23.732 | 16.672 | 2.516 | 1.00 | 52.89 |
| 1346 | CG2 | ILE | 167 | 24.814 | 17.546 | 3.159 | 1.00 | 52.89 |
| 1347 | CG1 | ILE | 167 | 23.884 | 15.227 | 2.997 | 1.00 | 52.89 |
| 1348 | CD1 | ILE | 167 | 25.008 | 14.493 | 2.326 | 1.00 | 52.89 |
| 1349 | C | ILE | 167 | 22.356 | 18.698 | 2.934 | 1.00 | 41.84 |
| 1350 | O | ILE | 167 | 22.535 | 19.347 | 1.905 | 1.00 | 41.84 |
| 1351 | N | THR | 168 | 22.194 | 19.250 | 4.132 | 1.00 | 49.92 |
| 1352 | CA | THR | 168 | 22.213 | 20.694 | 4.336 | 1.00 | 49.92 |
| 1353 | CB | THR | 168 | 20.999 | 21.151 | 5.122 | 1.00 | 52.27 |
| 1354 | OG1 | THR | 168 | 19.818 | 20.706 | 4.465 | 1.00 | 52.27 |
| 1355 | CG2 | THR | 168 | 20.977 | 22.658 | 5.238 | 1.00 | 52.27 |
| 1356 | C | THR | 168 | 23.434 | 21.157 | 5.126 | 1.00 | 49.92 |
| 1357 | O | THR | 168 | 23.768 | 20.577 | 6.156 | 1.00 | 49.92 |
| 1358 | N | VAL | 169 | 24.078 | 22.216 | 4.649 | 1.00 | 52.96 |
| 1359 | CA | VAL | 169 | 25.230 | 22.791 | 5.331 | 1.00 | 52.96 |
| 1360 | CB | VAL | 169 | 26.359 | 23.106 | 4.346 | 1.00 | 44.29 |
| 1361 | CG1 | VAL | 169 | 27.578 | 23.646 | 5.112 | 1.00 | 44.29 |
| 1362 | CG2 | VAL | 169 | 26.710 | 21.865 | 3.560 | 1.00 | 44.29 |
| 1363 | C | VAL | 169 | 24.837 | 24.102 | 6.037 | 1.00 | 52.96 |
| 1364 | O | VAL | 169 | 24.456 | 25.058 | 5.373 | 1.00 | 52.96 |
| 1365 | N | ILE | 170 | 24.920 | 24.138 | 7.372 | 1.00 | 52.65 |
| 1366 | CA | ILE | 170 | 24.585 | 25.345 | 8.146 | 1.00 | 52.65 |
| 1367 | CB | ILE | 170 | 23.700 | 25.033 | 9.380 | 1.00 | 54.27 |
| 1368 | CG2 | ILE | 170 | 22.411 | 24.342 | 8.945 | 1.00 | 54.27 |
| 1369 | CG1 | ILE | 170 | 24.473 | 24.181 | 10.390 | 1.00 | 54.27 |
| 1370 | CD1 | ILE | 170 | 23.644 | 23.798 | 11.619 | 1.00 | 54.27 |
| 1371 | C | ILE | 170 | 25.841 | 26.060 | 8.638 | 1.00 | 52.65 |
| 1372 | O | ILE | 170 | 26.931 | 25.488 | 8.620 | 1.00 | 52.65 |
| 1373 | N | LYS | 171 | 25.697 | 27.308 | 9.075 | 1.00 | 92.13 |
| 1374 | CA | LYS | 171 | 26.849 | 28.070 | 9.551 | 1.00 | 92.13 |
| 1375 | CB | LYS | 171 | 26.566 | 29.574 | 9.470 | 1.00 | 112.78 |
| 1376 | CG | LYS | 171 | 27.788 | 30.436 | 9.745 | 1.00 | 112.78 |
| 1377 | CD | LYS | 171 | 27.599 | 31.881 | 9.293 | 1.00 | 112.78 |
| 1378 | CE | LYS | 171 | 27.658 | 32.008 | 7.772 | 1.00 | 112.78 |
| 1379 | NZ | LYS | 171 | 27.643 | 33.430 | 7.310 | 1.00 | 112.78 |
| 1380 | C | LYS | 171 | 27.244 | 27.674 | 10.974 | 1.00 | 92.13 |
| 1381 | O | LYS | 171 | 26.388 | 27.384 | 11.812 | 1.00 | 92.13 |
| 1382 | N | ALA | 172 | 28.551 | 27.662 | 11.230 | 1.00 | 124.64 |
| 1383 | CA | ALA | 172 | 29.108 | 27.282 | 12.529 | 1.00 | 124.64 |
| 1384 | CB | ALA | 172 | 30.617 | 27.553 | 12.537 | 1.00 | 104.18 |
| 1385 | C | ALA | 172 | 28.457 | 27.910 | 13.772 | 1.00 | 124.64 |
| 1386 | O | ALA | 172 | 28.071 | 27.191 | 14.695 | 1.00 | 124.64 |
| 1387 | N | PRO | 173 | 28.337 | 29.254 | 13.819 | 1.00 | 141.76 |
| 1388 | CD | PRO | 173 | 28.819 | 30.230 | 12.825 | 1.00 | 113.27 |
| 1389 | CA | PRO | 173 | 27.730 | 29.953 | 14.963 | 1.00 | 141.76 |
| 1390 | CB | PRO | 173 | 27.492 | 31.354 | 14.415 | 1.00 | 113.27 |
| 1391 | CG | PRO | 173 | 28.701 | 31.556 | 13.575 | 1.00 | 113.27 |
| 1392 | C | PRO | 173 | 26.461 | 29.332 | 15.553 | 1.00 | 141.76 |
| 1393 | O | PRO | 173 | 25.733 | 28.603 | 14.876 | 1.00 | 141.76 |
| 1394 | N | ARG | 174 | 26.219 | 29.644 | 16.826 | 1.00 | 135.93 |
| 1395 | CA | ARG | 174 | 25.070 | 29.155 | 17.592 | 1.00 | 135.93 |
| 1396 | CB | ARG | 174 | 24.358 | 30.340 | 18.256 | 1.00 | 141.76 |
| 1397 | CG | ARG | 174 | 25.304 | 31.216 | 19.065 | 1.00 | 141.76 |
| 1398 | CD | ARG | 174 | 24.573 | 32.197 | 19.965 | 1.00 | 141.76 |
| 1399 | NE | ARG | 174 | 25.519 | 32.986 | 20.755 | 1.00 | 141.76 |
| 1400 | CZ | ARG | 174 | 25.178 | 33.813 | 21.741 | 1.00 | 141.76 |
| 1401 | NH1 | ARG | 174 | 23.901 | 33.970 | 22.071 | 1.00 | 141.76 |
| 1402 | NH2 | ARG | 174 | 26.115 | 34.484 | 22.400 | 1.00 | 141.76 |
| 1403 | C | ARG | 174 | 24.068 | 28.322 | 16.796 | 1.00 | 135.93 |
| 1404 | O | ARG | 174 | 24.026 | 27.095 | 16.923 | 1.00 | 135.93 |
| 1405 | C1 | NAG | 21A | 25.553 | −8.090 | 14.864 | 1.00 | 113.42 |
| 1406 | C2 | NAG | 21A | 26.103 | −8.923 | 13.694 | 1.00 | 113.42 |
| 1407 | N2 | NAG | 21A | 25.455 | −8.533 | 12.455 | 1.00 | 113.42 |
| 1408 | C7 | NAG | 21A | 26.186 | −8.153 | 11.409 | 1.00 | 113.42 |
| 1409 | O7 | NAG | 21A | 27.417 | −8.115 | 11.428 | 1.00 | 113.42 |
| 1410 | C8 | NAG | 21A | 25.436 | −7.756 | 10.148 | 1.00 | 113.42 |
| 1411 | C3 | NAG | 21A | 25.876 | −10.419 | 13.955 | 1.00 | 113.42 |
| 1412 | O3 | NAG | 21A | 26.513 | −11.185 | 12.940 | 1.00 | 113.42 |
| 1413 | C4 | NAG | 21A | 26.441 | −10.817 | 15.323 | 1.00 | 113.42 |
| 1414 | O4 | NAG | 21A | 26.084 | −12.164 | 15.616 | 1.00 | 113.42 |
| 1415 | C5 | NAG | 21A | 25.905 | −9.887 | 16.423 | 1.00 | 113.42 |
| 1416 | O5 | NAG | 21A | 26.175 | −8.502 | 16.092 | 1.00 | 113.42 |
| 1417 | C6 | NAG | 21A | 26.569 | −10.164 | 17.760 | 1.00 | 113.42 |
| 1418 | O6 | NAG | 21A | 26.198 | −9.199 | 18.732 | 1.00 | 113.42 |
| 1419 | C1 | NAG | 42A | 9.440 | 5.012 | 15.315 | 1.00 | 74.70 |
| 1420 | C2 | NAG | 42A | 8.867 | 3.648 | 14.939 | 1.00 | 74.70 |
| 1421 | N2 | NAG | 42A | 9.316 | 2.609 | 15.844 | 1.00 | 74.70 |
| 1422 | C7 | NAG | 42A | 8.618 | 2.342 | 16.941 | 1.00 | 74.70 |
| 1423 | O7 | NAG | 42A | 7.605 | 2.973 | 17.251 | 1.00 | 74.70 |
| 1424 | C8 | NAG | 42A | 9.129 | 1.223 | 17.840 | 1.00 | 74.70 |
| 1425 | C3 | NAG | 42A | 9.294 | 3.312 | 13.516 | 1.00 | 74.70 |
| 1426 | O3 | NAG | 42A | 8.752 | 2.058 | 13.131 | 1.00 | 74.70 |
| 1427 | C4 | NAG | 42A | 8.835 | 4.399 | 12.538 | 1.00 | 74.70 |
| 1428 | O4 | NAG | 42A | 9.469 | 4.168 | 11.266 | 1.00 | 74.70 |
| 1429 | C5 | NAG | 42A | 9.262 | 5.795 | 13.046 | 1.00 | 74.70 |
| 1430 | O5 | NAG | 42A | 8.894 | 6.001 | 14.433 | 1.00 | 74.70 |
| 1431 | C6 | NAG | 42A | 8.596 | 6.900 | 12.259 | 1.00 | 74.70 |
| 1432 | O6 | NAG | 42A | 9.556 | 7.808 | 11.744 | 1.00 | 74.70 |
| 1433 | C1 | NAG | 42B | 8.771 | 3.603 | 10.203 | 1.00 | 81.02 |
| 1434 | C2 | NAG | 42B | 9.620 | 3.832 | 8.945 | 1.00 | 81.02 |
| 1435 | N2 | NAG | 42B | 9.736 | 5.248 | 8.651 | 1.00 | 81.02 |
| 1436 | C7 | NAG | 42B | 10.935 | 5.828 | 8.641 | 1.00 | 81.02 |
| 1437 | O7 | NAG | 42B | 11.980 | 5.214 | 8.866 | 1.00 | 81.02 |
| 1438 | C8 | NAG | 42B | 10.986 | 7.317 | 8.327 | 1.00 | 81.02 |
| 1439 | C3 | NAG | 42B | 9.064 | 3.068 | 7.750 | 1.00 | 81.02 |
| 1440 | O3 | NAG | 42B | 9.888 | 3.298 | 6.616 | 1.00 | 81.02 |
| 1441 | C4 | NAG | 42B | 9.103 | 1.604 | 8.138 | 1.00 | 81.02 |
| 1442 | O4 | NAG | 42B | 8.834 | 0.730 | 7.000 | 1.00 | 81.02 |
| 1443 | C5 | NAG | 42B | 8.162 | 1.393 | 9.341 | 1.00 | 81.02 |
| 1444 | O5 | NAG | 42B | 8.628 | 2.187 | 10.472 | 1.00 | 81.02 |
| 1445 | C6 | NAG | 42B | 8.140 | −0.057 | 9.812 | 1.00 | 81.02 |
| 1446 | O6 | NAG | 42B | 7.263 | −0.235 | 10.916 | 1.00 | 81.02 |
| 1447 | C1 | MAN | 42C | 7.548 | 0.362 | 6.612 | 1.00 | 121.66 |
| 1448 | C2 | MAN | 42C | 7.465 | 0.370 | 5.065 | 1.00 | 121.66 |
| 1449 | O2 | MAN | 42C | 8.504 | 1.176 | 4.523 | 1.00 | 121.66 |
| 1450 | C3 | MAN | 42C | 7.571 | −1.048 | 4.480 | 1.00 | 121.66 |
| 1451 | O3 | MAN | 42C | 8.850 | −1.599 | 4.759 | 1.00 | 121.66 |
| 1452 | C4 | MAN | 42C | 6.480 | −1.965 | 5.048 | 1.00 | 121.66 |
| 1453 | O4 | MAN | 42C | 5.296 | −1.845 | 4.272 | 1.00 | 121.66 |
| 1454 | C5 | MAN | 42C | 6.167 | −1.621 | 6.510 | 1.00 | 121.66 |
| 1455 | O5 | MAN | 42C | 7.300 | −0.964 | 7.127 | 1.00 | 121.66 |
| 1456 | C6 | MAN | 42C | 5.858 | −2.862 | 7.336 | 1.00 | 121.66 |
| 1457 | O6 | MAN | 42C | 5.372 | −3.923 | 6.522 | 1.00 | 121.66 |
| 1458 | C1 | NAG | 166A | 14.879 | 16.481 | −0.659 | 1.00 | 69.14 |
| 1459 | C2 | NAG | 166A | 13.401 | 16.282 | −0.279 | 1.00 | 69.14 |
| 1460 | N2 | NAG | 166A | 13.208 | 14.952 | 0.269 | 1.00 | 69.14 |
| 1461 | C7 | NAG | 166A | 12.951 | 14.790 | 1.565 | 1.00 | 69.14 |
| 1462 | O7 | NAG | 166A | 12.855 | 15.734 | 2.356 | 1.00 | 69.14 |
| 1463 | C8 | NAG | 166A | 12.765 | 13.364 | 2.065 | 1.00 | 69.14 |
| 1464 | C3 | NAG | 166A | 12.515 | 16.472 | −1.519 | 1.00 | 69.14 |
| 1465 | O3 | NAG | 166A | 11.139 | 16.439 | −1.147 | 1.00 | 69.14 |
| 1466 | C4 | NAG | 166A | 12.831 | 17.806 | −2.209 | 1.00 | 69.14 |
| 1467 | O4 | NAG | 166A | 12.124 | 17.873 | −3.464 | 1.00 | 69.14 |
| 1468 | C5 | NAG | 166A | 14.346 | 17.962 | −2.463 | 1.00 | 69.14 |
| 1469 | O5 | NAG | 166A | 15.072 | 17.789 | −1.224 | 1.00 | 69.14 |
| 1470 | C6 | NAG | 166A | 14.736 | 19.321 | −3.074 | 1.00 | 69.14 |
| 1471 | O6 | NAG | 166A | 15.449 | 20.162 | −2.169 | 1.00 | 69.14 |
| 1472 | C1 | NAG | 166B | 11.515 | 19.084 | −3.754 | 1.00 | 88.70 |
| 1473 | C2 | NAG | 166B | 11.108 | 19.132 | −5.235 | 1.00 | 88.70 |
| 1474 | N2 | NAG | 166B | 12.288 | 19.054 | −6.081 | 1.00 | 88.70 |
| 1475 | C7 | NAG | 166B | 12.566 | 17.929 | −6.736 | 1.00 | 88.70 |
| 1476 | O7 | NAG | 166B | 11.857 | 16.927 | −6.667 | 1.00 | 88.70 |
| 1477 | C8 | NAG | 166B | 13.816 | 17.904 | −7.601 | 1.00 | 88.70 |
| 1478 | C3 | NAG | 166B | 10.337 | 20.432 | −5.516 | 1.00 | 88.70 |
| 1479 | O3 | NAG | 166B | 9.844 | 20.426 | −6.848 | 1.00 | 88.70 |
| 1480 | C4 | NAG | 166B | 9.165 | 20.603 | −4.535 | 1.00 | 88.70 |
| 1481 | O4 | NAG | 166B | 8.572 | 21.908 | −4.731 | 1.00 | 88.70 |
| 1482 | C5 | NAG | 166B | 9.688 | 20.469 | −3.089 | 1.00 | 88.70 |
| 1483 | O5 | NAG | 166B | 10.358 | 19.203 | −2.919 | 1.00 | 88.70 |
| 1484 | C6 | NAG | 166B | 8.612 | 20.538 | −2.021 | 1.00 | 88.70 |
| 1485 | O6 | NAG | 166B | 9.186 | 20.529 | −0.721 | 1.00 | 88.70 |
| 1486 | C1 | MAN | 166C | 7.210 | 22.047 | −4.475 | 1.00 | 140.23 |
| 1487 | C2 | MAN | 166C | 6.971 | 23.248 | −3.529 | 1.00 | 140.23 |
| 1488 | O2 | MAN | 166C | 8.186 | 23.629 | −2.897 | 1.00 | 140.23 |
| 1489 | C3 | MAN | 166C | 6.384 | 24.444 | −4.292 | 1.00 | 140.23 |
| 1490 | O3 | MAN | 166C | 7.294 | 24.880 | −5.294 | 1.00 | 140.23 |
| 1491 | C4 | MAN | 166C | 5.054 | 24.047 | −4.942 | 1.00 | 140.23 |
| 1492 | O4 | MAN | 166C | 4.019 | 24.073 | −3.966 | 1.00 | 140.23 |
| 1493 | C5 | MAN | 166C | 5.141 | 22.640 | −5.572 | 1.00 | 140.23 |
| 1494 | O5 | MAN | 166C | 6.527 | 22.236 | −5.734 | 1.00 | 140.23 |
| 1495 | C6 | MAN | 166C | 4.497 | 22.590 | −6.946 | 1.00 | 140.23 |
| 1496 | O6 | MAN | 166C | 3.935 | 21.313 | −7.207 | 1.00 | 140.23 |
| 1497 | OH2 | WAT | 1000 | 17.505 | 20.612 | −1.007 | 1.00 | 68.91 |
| 1498 | OH2 | WAT | 1001 | 8.876 | 15.888 | −2.154 | 1.00 | 68.91 |
| 1499 | OH2 | WAT | 1002 | 24.042 | 8.073 | 7.063 | 1.00 | 68.91 |
| 1500 | OH2 | WAT | 1003 | 18.824 | 3.262 | −1.304 | 1.00 | 68.91 |
| 1501 | OH2 | WAT | 1004 | 30.337 | −6.784 | −6.997 | 1.00 | 68.91 |
| 1502 | OH2 | WAT | 1005 | 23.648 | −7.978 | −9.801 | 1.00 | 68.91 |
| 1503 | OH2 | WAT | 1006 | 15.659 | −8.042 | 14.310 | 1.00 | 68.91 |
| 1504 | OH2 | WAT | 1007 | 20.414 | 5.554 | −0.296 | 1.00 | 68.91 |
| 1505 | OH2 | WAT | 1008 | 25.967 | 2.758 | 12.004 | 1.00 | 68.91 |
| 1506 | OH2 | WAT | 1009 | 15.148 | 17.603 | 2.679 | 1.00 | 68.91 |
| 1507 | OH2 | WAT | 1010 | 20.894 | 14.371 | −7.289 | 1.00 | 68.91 |
| 1508 | OH2 | WAT | 1011 | 29.583 | −2.803 | 0.523 | 1.00 | 68.91 |
| 1509 | OH2 | WAT | 1012 | 23.414 | −6.190 | 4.824 | 1.00 | 68.91 |
| 1510 | OH2 | WAT | 1013 | 15.450 | 4.228 | 29.002 | 1.00 | 68.91 |
| 1511 | OH2 | WAT | 1014 | 20.819 | 19.173 | 25.674 | 1.00 | 68.91 |
| 1512 | OH2 | WAT | 1015 | 26.533 | −12.922 | −8.874 | 1.00 | 68.91 |
| 1513 | OH2 | WAT | 1016 | 20.297 | 0.066 | −4.940 | 1.00 | 68.91 |
| 1514 | OH2 | WAT | 1017 | 12.264 | 10.290 | 21.606 | 1.00 | 68.91 |
| 1515 | OH2 | WAT | 1018 | 10.662 | 12.690 | 26.479 | 1.00 | 68.91 |
| 1516 | OH2 | WAT | 1019 | 30.520 | 28.860 | 10.139 | 1.00 | 68.91 |
| 1517 | OH2 | WAT | 1020 | 10.314 | 0.397 | 3.316 | 1.00 | 68.91 |
| 1518 | OH2 | WAT | 1021 | 29.439 | 18.571 | −2.756 | 1.00 | 68.91 |
| 1519 | OH2 | WAT | 1022 | 35.124 | 0.026 | −10.508 | 1.00 | 68.91 |
| 1520 | OH2 | WAT | 1023 | 26.056 | 0.085 | 8.311 | 1.00 | 68.91 |
| 1521 | OH2 | WAT | 1024 | 29.558 | 14.948 | 9.236 | 1.00 | 68.91 |
| 1522 | OH2 | WAT | 1025 | 28.174 | 4.087 | −11.726 | 1.00 | 68.91 |
| 1523 | OH2 | WAT | 1026 | 9.612 | 1.088 | 0.709 | 1.00 | 68.91 |
| 1524 | OH2 | WAT | 1027 | 28.026 | 4.309 | 20.417 | 1.00 | 68.91 |
| 1525 | OH2 | WAT | 1028 | 25.503 | 9.375 | 10.445 | 1.00 | 68.91 |
| 1526 | OH2 | WAT | 1029 | 16.927 | 10.725 | −7.396 | 1.00 | 68.91 |
| 1527 | OH2 | WAT | 1030 | 32.003 | 6.822 | 32.047 | 1.00 | 68.91 |
| 1528 | OH2 | WAT | 1031 | 12.422 | 0.452 | 21.294 | 1.00 | 68.91 |
| 1529 | OH2 | WAT | 1032 | 15.327 | 0.065 | 19.129 | 1.00 | 68.91 |
| 1530 | OH2 | WAT | 1033 | 11.536 | 8.204 | 33.994 | 1.00 | 68.91 |
| 1531 | OH2 | WAT | 1034 | 18.003 | 7.978 | −6.726 | 1.00 | 68.91 |
| 1532 | OH2 | WAT | 1035 | 34.477 | 2.731 | −7.719 | 1.00 | 68.91 |
| 1533 | OH2 | WAT | 1036 | 25.373 | 34.820 | 8.269 | 1.00 | 68.91 |
| 1534 | OH2 | WAT | 1037 | 14.026 | 16.389 | 25.301 | 1.00 | 68.91 |
| 1535 | OH2 | WAT | 1038 | 30.733 | 30.153 | 16.022 | 1.00 | 68.91 |
| 1536 | OH2 | WAT | 1039 | 25.276 | 21.121 | −10.191 | 1.00 | 68.91 |
| 1537 | OH2 | WAT | 1040 | 16.971 | 8.768 | −11.221 | 1.00 | 68.91 |
| 1538 | OH2 | WAT | 1041 | 26.997 | 12.580 | 36.282 | 1.00 | 68.91 |
| 1539 | OH2 | WAT | 1042 | 5.954 | 6.575 | 17.557 | 1.00 | 68.91 |
| 1540 | OH2 | WAT | 1043 | 26.429 | −14.196 | 14.154 | 1.00 | 68.91 |
| 1541 | OH2 | WAT | 1044 | 41.801 | 6.111 | −5.021 | 1.00 | 68.91 |
| 1542 | OH2 | WAT | 1045 | 16.712 | 8.152 | 1.031 | 1.00 | 68.91 |
| 1543 | OH2 | WAT | 1046 | 10.222 | 17.172 | 0.994 | 1.00 | 68.91 |
| 1544 | OH2 | WAT | 1047 | 26.531 | 8.260 | 28.436 | 1.00 | 68.91 |
| 1545 | OH2 | WAT | 1048 | 17.529 | 12.929 | 2.834 | 1.00 | 68.91 |
| 1546 | OH2 | WAT | 1049 | 31.571 | 12.227 | −10.072 | 1.00 | 68.91 |
| 1547 | OH2 | WAT | 1050 | 22.536 | 1.995 | 35.016 | 1.00 | 68.91 |
| 1548 | OH2 | WAT | 1051 | 26.121 | 6.724 | −12.642 | 1.00 | 68.91 |
| 1549 | OH2 | WAT | 1052 | 14.788 | 0.096 | 2.327 | 1.00 | 68.91 |
| 1550 | OH2 | WAT | 1053 | 36.387 | 12.151 | −8.959 | 1.00 | 68.91 |
| 1551 | OH2 | WAT | 1054 | 30.213 | −9.146 | −4.152 | 1.00 | 68.91 |
| 1552 | OH2 | WAT | 1055 | 33.615 | 21.863 | −0.263 | 1.00 | 68.91 |
| 1553 | OH2 | WAT | 1056 | 10.283 | −4.295 | 32.761 | 1.00 | 68.91 |
| 1554 | OH2 | WAT | 1057 | 28.514 | 0.501 | −14.456 | 1.00 | 68.91 |
| 1555 | OH2 | WAT | 1058 | 16.608 | −5.089 | 16.354 | 1.00 | 68.91 |
| 1556 | OH2 | WAT | 1059 | 32.212 | −2.748 | 2.548 | 1.00 | 68.91 |
| 1557 | OH2 | WAT | 1060 | 28.253 | −14.928 | −6.193 | 1.00 | 68.91 |
| 1558 | OH2 | WAT | 1061 | 22.375 | 14.011 | 20.937 | 1.00 | 68.91 |
| 1559 | OH2 | WAT | 1062 | 17.962 | −4.643 | 18.605 | 1.00 | 68.91 |
| 1560 | OH2 | WAT | 1063 | 33.412 | 17.614 | 12.726 | 1.00 | 68.91 |
| 1561 | OH2 | WAT | 1064 | 14.403 | 13.829 | 5.224 | 1.00 | 68.91 |
| 1562 | OH2 | WAT | 1065 | 22.334 | 16.845 | 22.648 | 1.00 | 68.91 |
| 1563 | OH2 | WAT | 1066 | 3.946 | −0.489 | 7.854 | 1.00 | 68.91 |
| 1564 | OH2 | WAT | 1067 | 19.383 | 17.873 | 5.189 | 1.00 | 68.91 |
| 1565 | OH2 | WAT | 1068 | 15.472 | 16.647 | 23.054 | 1.00 | 68.91 |
| 1566 | OH2 | WAT | 1069 | 29.541 | 28.573 | 2.954 | 1.00 | 68.91 |
| 1567 | OH2 | WAT | 1070 | 22.439 | 9.086 | 32.823 | 1.00 | 68.91 |
| 1568 | OH2 | WAT | 1071 | 12.994 | 2.582 | 4.613 | 1.00 | 68.91 |
| 1569 | OH2 | WAT | 1072 | 8.173 | −4.098 | 4.759 | 1.00 | 68.91 |
| 1570 | OH2 | WAT | 1073 | 6.843 | 21.529 | −8.563 | 1.00 | 68.91 |
| 1571 | OH2 | WAT | 1074 | 6.493 | 8.743 | 13.308 | 1.00 | 68.91 |
| 1572 | OH2 | WAT | 1075 | 38.018 | 4.521 | −0.320 | 1.00 | 68.91 |
| 1573 | OH2 | WAT | 1076 | 24.471 | −3.010 | 18.115 | 1.00 | 68.91 |
| 1574 | OH2 | WAT | 1077 | 25.888 | −4.454 | 10.596 | 1.00 | 68.91 |
| 1575 | OH2 | WAT | 1078 | 14.459 | 7.299 | −5.712 | 1.00 | 68.91 |
| 1576 | OH2 | WAT | 1079 | 29.390 | 19.413 | 11.601 | 1.00 | 68.91 |
| 1577 | OH2 | WAT | 1080 | 20.808 | 23.774 | 28.950 | 1.00 | 68.91 |
| 1578 | OH2 | WAT | 1081 | 30.321 | 32.666 | 4.517 | 1.00 | 68.91 |
| 1579 | OH2 | WAT | 1082 | 18.638 | 14.702 | 5.513 | 1.00 | 68.91 |
| 1580 | OH2 | WAT | 1083 | 10.393 | 2.751 | 24.212 | 1.00 | 68.91 |
| 1581 | OH2 | WAT | 1084 | 34.357 | 8.750 | 4.350 | 1.00 | 68.91 |
| 1582 | OH2 | WAT | 1085 | 38.981 | 27.376 | 6.226 | 1.00 | 68.91 |
| 1583 | OH2 | WAT | 1086 | 13.633 | −5.771 | 10.421 | 1.00 | 68.91 |
| 1584 | OH2 | WAT | 1087 | 30.187 | −0.118 | 1.986 | 1.00 | 68.91 |
| 1585 | OH2 | WAT | 1088 | 19.984 | 12.423 | 13.551 | 1.00 | 68.91 |
| 1586 | OH2 | WAT | 1089 | 33.138 | 0.672 | 3.694 | 1.00 | 68.91 |
| 1587 | OH2 | WAT | 1090 | 22.605 | 13.264 | 0.581 | 1.00 | 68.91 |
| 1588 | OH2 | WAT | 1091 | 14.668 | 10.306 | 8.575 | 1.00 | 68.91 |
| 1589 | OH2 | WAT | 1092 | 21.896 | 16.105 | 11.480 | 1.00 | 68.91 |
| 1590 | OH2 | WAT | 1093 | 26.996 | 0.604 | 11.132 | 1.00 | 68.91 |
| 1591 | OH2 | WAT | 1094 | 31.571 | 7.546 | 16.430 | 1.00 | 68.91 |
| 1592 | OH2 | WAT | 1095 | 30.193 | 3.267 | −18.033 | 1.00 | 68.91 |
| 1593 | OH2 | WAT | 1096 | 30.112 | 6.862 | 20.521 | 1.00 | 68.91 |
| 1594 | OH2 | WAT | 1097 | 25.159 | 32.416 | 11.157 | 1.00 | 68.91 |
| 1595 | OH2 | WAT | 1098 | 25.354 | −13.410 | 18.368 | 1.00 | 68.91 |
| 1596 | OH2 | WAT | 1099 | 20.969 | −1.882 | 24.389 | 1.00 | 68.91 |
| 1597 | OH2 | WAT | 1100 | 32.515 | −1.311 | −2.770 | 1.00 | 68.91 |
| 1598 | OH2 | WAT | 1101 | 30.357 | 10.302 | −14.689 | 1.00 | 68.91 |
| 1599 | OH2 | WAT | 1102 | 30.517 | 8.184 | 27.857 | 1.00 | 68.91 |
| 1600 | OH2 | WAT | 1103 | 13.656 | −2.654 | 31.941 | 1.00 | 68.91 |
| 1601 | OH2 | WAT | 1104 | 15.222 | 19.539 | 18.640 | 1.00 | 68.91 |
| 1602 | OH2 | WAT | 1105 | 34.184 | 25.830 | 5.139 | 1.00 | 68.91 |
| 1603 | OH2 | WAT | 1106 | 27.056 | 25.512 | 13.333 | 1.00 | 68.91 |
| 1604 | OH2 | WAT | 1107 | 33.492 | 6.985 | −2.929 | 1.00 | 68.91 |
| 1605 | OH2 | WAT | 1108 | 12.951 | 8.497 | 11.009 | 1.00 | 68.91 |
| 1606 | OH2 | WAT | 1109 | 23.498 | 11.331 | 13.153 | 1.00 | 68.91 |
| 1607 | OH2 | WAT | 1110 | 29.557 | −10.045 | 18.238 | 1.00 | 68.91 |
| 1608 | OH2 | WAT | 1111 | 29.239 | 18.077 | −10.203 | 1.00 | 68.91 |
| 1609 | OH2 | WAT | 1112 | 20.316 | 12.553 | −11.333 | 1.00 | 68.91 |
| 1610 | OH2 | WAT | 1113 | 27.872 | 2.853 | 33.575 | 1.00 | 68.91 |
| 1611 | OH2 | WAT | 1114 | 21.439 | 20.739 | −11.349 | 1.00 | 68.91 |
| 1612 | OH2 | WAT | 1115 | 34.052 | 2.985 | 36.842 | 1.00 | 68.91 |
| 1613 | OH2 | WAT | 1116 | 11.123 | −3.141 | 18.133 | 1.00 | 68.91 |
| 1614 | OH2 | WAT | 1117 | 10.985 | 13.263 | 12.061 | 1.00 | 68.91 |
| 1615 | OH2 | WAT | 1118 | 33.767 | 28.659 | −2.115 | 1.00 | 68.91 |
| 1616 | OH2 | WAT | 1119 | 23.247 | 24.523 | 18.586 | 1.00 | 68.91 |
| 1617 | OH2 | WAT | 1120 | 31.382 | 23.627 | 14.310 | 1.00 | 68.91 |
| 1618 | OH2 | WAT | 1121 | 12.025 | −1.649 | 0.565 | 1.00 | 68.91 |
| 1619 | OH2 | WAT | 1122 | 9.969 | 2.385 | 20.835 | 1.00 | 68.91 |
| 1620 | OH2 | WAT | 1123 | 20.360 | −3.059 | −13.904 | 1.00 | 68.91 |

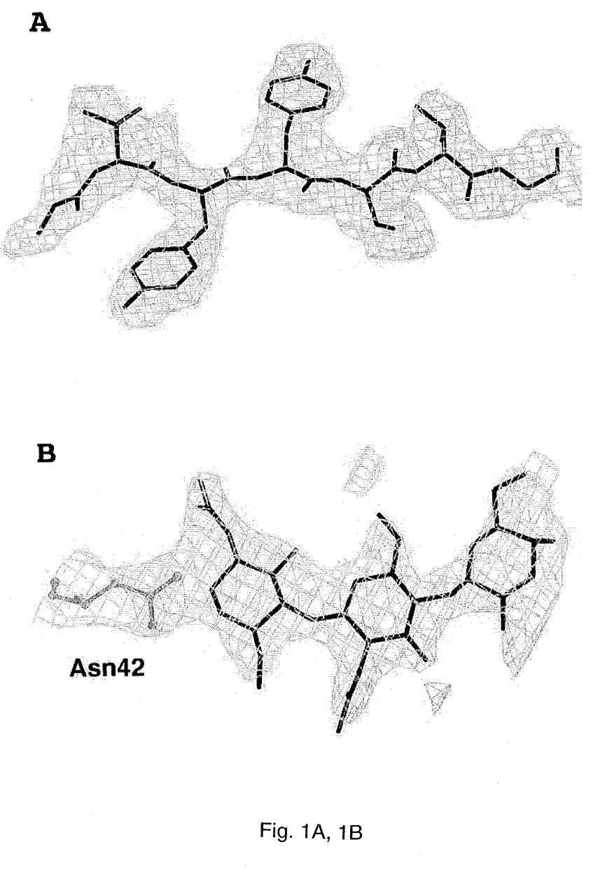

As used herein, an atomic coordinate, also referred to herein as a structure coordinate or coordinate, is a mathematical coordinate derived from mathematical equations related to the patterns obtained on diffraction of X-rays by the atoms of a protein crystal. The diffraction data are typically used to calculate an electron density map, such as that shown in FIG. 1, which is used to establish the positions of the individual atoms within the unit cell of the crystal. A model that substantially represents the atomic coordinates specified in Table 1 includes not only models that literally represent the coordinates but also models representing a coordinate transformation of such atomic coordinates, for example, by changing the spatial orientation of the coordinates.

Additional embodiments of the present invention include 3-D models of extracellular domains of FcεRIα proteins that substantially represent the atomic coordinates specified in Table 5, Table 6, Table 7 or Table 8, each of which is at the end of the Examples section. Similarly, a model that substantially represents the atomic coordinates specified in Table 5, Table 6, Table 7 or Table 8 includes not only models that literally represent the coordinates but also models representing a coordinate transformation of such atomic coordinates.

The present invention also includes a 3-D model that is a modification of a 3-D model that substantially represents the atomic coordinates specified in Table 5, Table 6, Table 7 or Table 8. As used herein, a modification, also referred to herein as a model modification, is a model that represents a protein that binds to a Fc domain of an antibody. A model modification includes, but is not limited to: a refinement of the model that substantially represents the atomic coordinates specified in Table 1, Table 5, Table 6, Table 7 or Table 8; a model representing any fragment of a protein having the atomic coordinates specified in Table 1, Table 5, Table 6, Table 7 or Table 8 that binds to a Fc domain of an antibody; a model based on other FcεRIα protein crystals, such as a model based on one or more of the crystals disclosed in the Examples; a model produced using homology modeling techniques to, for example, incorporate all or any part of the amino acid sequence of another FcR into a 3-D model of the extracellular domain of the model substantially representing the atomic coordinates specified in Table 1, Table 5, Table 6, Table 7 or Table 8 or incorporate all or any part of the amino acid sequence of a FcεRIα protein into a 3-D model of another FcR; and a modification representing a FcR with an altered function, which preferably can be used to design a mutein with an improved function compared to an unmodified protein. As used herein, the term unmodified protein refers to a protein that has not been intentionally subjected to either random or site-directed (i.e., targeted) mutagenesis.

A model of the present invention can be represented in a variety of forms including, but not limited to, listing the coordinates of all atoms comprising the model, providing a physical 3-D model, imaging the model on a computer screen, providing a picture of said model, and deriving a set of coordinates based of a picture of the model, for example by extracting coordinates from a picture or placing a similar immunoglobulin domain into the 3-D model of human FcεRIα₁₋₁₇₆ protein form M1, FcεRIα₁₋₁₇₆ protein form M2, FcεRIα₁₋₁₇₂ protein form T1, FcεRIα₁₋₁₇₂ protein form T2, or FcεRIα₁₋₁₇₂ protein form H1 and deriving a model of the similar domain. Physical 3-D models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Evans and Sutherland, Salt Lake City, Utah, Biosym Technologies, San Diego, Calif., Tripos, Inc., and Molecular Simulations Inc. The phrase “providing a picture of the model” refers to the ability to generate a “hard copy” of the model. Hard copies include both motion and still pictures. Computer screen images and pictures of the model can be visualized in a number of formats including, but not limited to, electron density maps, ribbon diagrams, space-filling representations, α carbon traces, topology diagrams, lists of interatomic vectors, phi/psi/chi angle representations of the coordinates, and contact maps, examples of some of which are in the Figs. Representations of the model can include the entire model or portions thereof.

In one embodiment, a model of the present invention identifies the solvent accessibility of amino acid residues of the corresponding protein. The solvent accessibilities of the amino acids in human FcεRIα₁₋₁₇₆ protein (form M1) are indicated in Table 2.

TABLE 2
PhFcεRIα₁₋₁₇₆, Form M1, residue exposure
>>>> Surface plot for:
>>>> structure file = fcr10_gen.mtf
>>>> coordinate set = fcr10b.pdb

| resid | resname | access | access-main | access-side |
|---|---|---|---|---|
| 4 | LYS | 18.7522 | 5.5920 | 29.2803 |
| 5 | PRO | 0.5301 | 0.7105 | 0.2895 |
| 6 | LYS | 14.4465 | 0.5227 | 25.5856 |
| 7 | VAL | 1.6658 | 2.9151 | 0.0000 |
| 8 | SER | 10.6765 | 1.6199 | 28.7895 |
| 9 | LEU | 3.3901 | 4.3765 | 2.4038 |
| 10 | ASN | 12.4750 | 0.9379 | 24.0120 |
| 11 | PRO | 9.1378 | 0.1896 | 21.0688 |
| 12 | PRO | 10.7886 | 2.5914 | 21.7181 |
| 13 | TRP | 2.8040 | 0.1461 | 3.8672 |
| 14 | ASN | 2.8382 | 0.0019 | 5.6746 |
| 15 | ARG | 0.8717 | 0.0047 | 1.3672 |
| 16 | ILE | 0.8262 | 0.0000 | 1.6524 |
| 17 | PHE | 0.2251 | 0.0002 | 0.3536 |
| 18 | LYS | 10.3275 | 2.1781 | 16.8470 |
| 19 | GLY | 5.9941 | 5.9941 | 0.0000 |
| 20 | GLU | 3.4574 | 0.0003 | 6.2230 |
| 21 | ASN | 5.5027 | 3.1911 | 7.8142 |
| 22 | VAL | 0.4139 | 0.5396 | 0.2464 |
| 23 | THR | 5.3412 | 0.0611 | 12.3812 |
| 24 | LEU | 0.1383 | 0.0000 | 0.2767 |
| 25 | THR | 6.9459 | 0.0105 | 16.1931 |
| 26 | CYS | 0.2279 | 0.2962 | 0.0913 |
| 27 | ASN | 6.3601 | 2.3608 | 10.3594 |
| 28 | GLY | 15.2937 | 15.2937 | 0.0000 |
| 29 | ASN | 12.5836 | 3.3134 | 21.8538 |
| 30 | ASN | 2.9321 | 4.7397 | 1.1246 |
| 31 | PHE | 10.9538 | 0.4808 | 16.9384 |
| 32 | PHE | 16.8929 | 5.7840 | 23.2409 |
| 33 | GLU | 19.4108 | 11.1422 | 26.0256 |
| 34 | VAL | 10.7289 | 4.6702 | 18.8072 |
| 35 | SER | 2.4235 | 2.0900 | 3.0905 |
| 36 | SER | 13.8183 | 6.2435 | 28.9679 |
| 37 | THR | 0.2048 | 0.0825 | 0.3679 |
| 38 | LYS | 11.0359 | 0.0996 | 19.7850 |
| 39 | TRP | 0.0222 | 0.0000 | 0.0311 |
| 40 | PHE | 3.1821 | 0.0194 | 4.9894 |
| 41 | HIS | 3.3786 | 0.3964 | 5.3667 |
| 42 | ASN | 6.4876 | 7.0690 | 5.9062 |
| 43 | GLY | 10.7019 | 10.7019 | 0.0000 |
| 44 | SER | 11.7545 | 1.4355 | 32.3926 |
| 45 | LEU | 12.7619 | 7.2235 | 18.3003 |
| 46 | SER | 5.1618 | 3.6359 | 8.2137 |
| 47 | GLU | 18.9113 | 6.7955 | 28.6039 |
| 48 | GLU | 5.1912 | 1.8435 | 7.8693 |
| 49 | THR | 10.4814 | 0.7172 | 23.5005 |
| 50 | ASN | 12.2883 | 1.2937 | 23.2828 |
| 51 | SER | 7.5408 | 0.9771 | 20.6683 |
| 52 | SER | 5.9824 | 1.1729 | 15.6016 |
| 53 | LEU | 2.7948 | 0.0000 | 5.5895 |
| 54 | ASN | 11.0365 | 4.8824 | 17.1907 |
| 55 | iLE | 1.4787 | 1.1377 | 1.8197 |
| 56 | VAL | 10.1929 | 3.7822 | 18.7406 |
| 57 | ASN | 10.0544 | 0.9161 | 19.1928 |
| 58 | ALA | 0.4355 | 0.5444 | 0.0000 |
| 59 | LYS | 12.3709 | 0.0000 | 22.2676 |
| 60 | PHE | 3.8585 | 0.0995 | 6.0065 |
| 61 | GLU | 8.4358 | 0.0765 | 15.1232 |
| 62 | ASP | 3.5771 | 0.0000 | 7.1543 |
| 63 | SER | 0.1109 | 0.0000 | 0.3328 |
| 64 | GLY | 1.4454 | 1.4454 | 0.0000 |
| 65 | GLU | 3.8623 | 0.1172 | 6.8583 |
| 66 | TYR | 0.6305 | 0.0000 | 0.9458 |
| 67 | LYS | 5.0231 | 0.0000 | 9.0416 |
| 68 | CYS | 0.0000 | 0.0000 | 0.0000 |
| 69 | GLN | 4.0004 | 0.1217 | 7.1034 |
| 70 | HIS | 1.6360 | 1.2124 | 1.9183 |
| 71 | GLN | 12.0520 | 6.5738 | 16.4346 |
| 72 | GLN | 6.9718 | 4.8885 | 8.6385 |
| 73 | VAL | 18.2550 | 4.0583 | 37.1841 |
| 74 | ASN | 11.7258 | 0.8064 | 22.6451 |
| 75 | GLU | 8.0572 | 4.5805 | 10.8386 |
| 76 | SER | 1.1935 | 1.7903 | 0.0000 |
| 77 | GLU | 11.7837 | 0.3001 | 20.9705 |
| 78 | PRO | 6.8729 | 3.9043 | 10.8310 |
| 79 | VAL | 4.7487 | 0.8978 | 9.8832 |
| 80 | TYR | 10.6722 | 1.0753 | 15.4707 |
| 81 | LEU | 0.6889 | 1.0101 | 0.3678 |
| 82 | GLU | 6.0039 | 0.0005 | 10.8066 |
| 83 | VAL | 1.1805 | 2.0660 | 0.0000 |
| 84 | PHE | 3.1391 | 0.5957 | 4.5925 |
| 85 | SER | 11.3103 | 7.0817 | 19.7676 |
| 86 | ASP | 5.0469 | 1.8059 | 8.2880 |
| 87 | TRP | 8.7876 | 0.0000 | 12.3027 |
| 88 | LEU | 0.2129 | 0.4258 | 0.0000 |
| 89 | LEU | 0.4967 | 0.0525 | 0.9408 |
| 90 | LEU | 0.0300 | 0.0599 | 0.0000 |
| 91 | GLN | 0.1846 | 0.0000 | 0.3323 |
| 92 | ALA | 0.1116 | 0.1271 | 0.0495 |
| 93 | SER | 6.6376 | 5.5213 | 8.8700 |
| 94 | ALA | 6.8725 | 1.3918 | 28.7952 |
| 95 | GLU | 7.3784 | 1.6594 | 11.9535 |
| 96 | VAL | 11.5981 | 3.7388 | 22.0772 |
| 97 | VAL | 0.8323 | 0.7102 | 0.9951 |
| 98 | MET | 11.2704 | 0.4727 | 22.0682 |
| 99 | GLU | 9.0020 | 2.3489 | 14.3246 |
| 100 | GLY | 8.7203 | 8.7203 | 0.0000 |
| 101 | GLN | 10.5632 | 0.0000 | 19.0137 |
| 102 | PRO | 7.5364 | 2.1046 | 14.7788 |
| 103 | LEU | 0.0101 | 0.0065 | 0.0136 |
| 104 | PHE | 7.5886 | 0.0000 | 11.9250 |
| 105 | LEU | 0.0013 | 0.0000 | 0.0026 |
| 106 | ARG | 5.0182 | 0.0005 | 7.8855 |
| 107 | CYS | 0.1269 | 0.1901 | 0.0004 |
| 108 | HIS | 0.9132 | 0.3845 | 1.2657 |
| 109 | GLY | 0.5179 | 0.5179 | 0.0000 |
| 110 | TRP | 4.5690 | 0.0000 | 6.3966 |
| 111 | ARG | 16.0050 | 8.4847 | 20.3023 |
| 112 | ASN | 12.3469 | 5.3472 | 19.3466 |
| 113 | TRP | 5.4418 | 2.5536 | 6.5971 |
| 114 | ASP | 12.2436 | 2.6722 | 21.8150 |
| 115 | VAL | 1.0913 | 1.1789 | 0.9745 |
| 116 | TYR | 9.9588 | 0.0536 | 14.9114 |
| 117 | LYS | 15.8288 | 6.4497 | 23.3321 |
| 118 | VAL | 2.4049 | 3.9634 | 0.3269 |
| 119 | ILE | 7.4508 | 0.0000 | 14.9016 |
| 120 | TYR | 0.0000 | 0.0000 | 0.0000 |
| 121 | TYR | 3.5355 | 0.0193 | 5.2936 |
| 122 | LYS | 4.6755 | 0.3398 | 8.1440 |
| 123 | ASP | 10.1763 | 6.7061 | 13.6465 |
| 124 | GLY | 13.3789 | 13.3789 | 0.0000 |
| 125 | GLU | 13.2240 | 0.9044 | 23.0796 |
| 126 | ALA | 9.8218 | 3.5091 | 35.0725 |
| 127 | LEU | 2.8644 | 3.0445 | 2.6843 |
| 128 | LYS | 20.0249 | 8.2304 | 29.4606 |
| 129 | TYR | 9.3305 | 2.8367 | 12.5774 |
| 130 | TRP | 16.4879 | 6.2307 | 20.5908 |
| 131 | TYR | 3.4405 | 3.5735 | 3.3740 |
| 132 | GLU | 11.9086 | 2.0563 | 19.7905 |
| 133 | ASN | 9.2765 | 4.2727 | 14.2802 |
| 134 | HIS | 7.6393 | 0.0000 | 12.7321 |
| 135 | ASN | 8.0044 | 0.1229 | 15.8860 |
| 136 | ILE | 0.3804 | 0.3402 | 0.4205 |
| 137 | SER | 9.9436 | 6.1883 | 17.4541 |
| 138 | ILE | 0.9720 | 0.9189 | 1.0252 |
| 139 | THR | 14.4684 | 2.3046 | 30.6869 |
| 140 | ASN | 12.6642 | 3.2729 | 22.0554 |
| 141 | ALA | 0.2430 | 0.2930 | 0.0431 |
| 142 | THR | 6.7751 | 0.0000 | 15.8087 |
| 143 | VAL | 14.3987 | 1.2997 | 31.8640 |
| 144 | GLU | 14.4366 | 2.9912 | 23.5929 |
| 145 | ASP | 0.6429 | 0.0018 | 1.2841 |
| 146 | SER | 5.5523 | 1.9108 | 12.8352 |
| 147 | GLY | 4.1321 | 4.1321 | 0.0000 |
| 148 | THR | 4.1370 | 0.0488 | 9.5879 |
| 149 | TYR | 0.0265 | 0.0000 | 0.0398 |
| 150 | TYR | 3.8147 | 0.0000 | 5.7220 |
| 151 | CYS | 0.0000 | 0.0000 | 0.0000 |
| 152 | THR | 3.7177 | 0.0000 | 8.6747 |
| 153 | GLY | 0.4224 | 0.4224 | 0.0000 |
| 154 | LYS | 6.3203 | 0.0000 | 11.3765 |
| 155 | VAL | 0.0418 | 0.0267 | 0.0620 |
| 156 | TRP | 11.9658 | 3.7888 | 15.2367 |
| 157 | GLN | 15.4277 | 4.3561 | 24.2849 |
| 158 | LEU | 14.1140 | 0.4176 | 27.8104 |
| 159 | ASP | 13.2798 | 6.7381 | 19.8215 |
| 160 | TYR | 4.2173 | 2.1486 | 5.2517 |
| 161 | GLU | 11.5466 | 4.1966 | 17.4267 |
| 162 | SER | 0.5960 | 0.8940 | 0.0000 |
| 163 | GLU | 10.5746 | 0.2964 | 18.7972 |
| 164 | PRO | 11.0115 | 3.8863 | 20.5117 |
| 165 | LEU | 1.6740 | 0.6758 | 2.6721 |
| 166 | ASN | 5.2259 | 2.2692 | 8.1825 |
| 167 | ILE | 0.2968 | 0.5937 | 0.0000 |
| 168 | THR | 9.8239 | 0.0262 | 22.8875 |
| 169 | VAL | 1.6748 | 2.6882 | 0.3236 |
| 170 | ILE | 10.3926 | 1.8982 | 18.8869 |
| 171 | LYS | 15.1729 | 2.4981 | 25.3128 |
| 172 | ALA | 11.6822 | 3.6722 | 43.7220 |
| 173 | PRO | 13.4157 | 5.3766 | 24.1346 |
| 174 | ARG | 25.5533 | 20.1410 | 28.6460 |
| 21A | NAG | 17.8283 | 0.0000 | 17.8283 |
| 42A | NAG | 10.6799 | 0.0000 | 10.6799 |
| 42B | NAG | 8.9040 | 0.0000 | 8.9040 |
| 42C | MAN | 17.4386 | 0.0000 | 17.4386 |
| 166A | NAG | 16.8280 | 0.0000 | 16.8280 |
| 166B | NAG | 16.9174 | 0.0000 | 16.9174 |
| 166C | MAN | 21.1827 | 0.0000 | 21.1827 |

The solvent accessibilities of the amino acids in human FcεRIα protein forms T1, T2, M2 and M1 are indicated in Tables 9, 10, 11, and 12 respectively, each of which is at the end of the Examples section.

Residues that are solvent accessible are important as they represent amino acids that are on the external surface of the protein and, as such, may be involved in binding of a FcR to an antibody and as such be useful in designing proteins with an enhanced binding activity or in identifying compounds that inhibit such binding. In addition, solvent accessible residues can represent targets for modification to produce a FcR with improved function. Such analysis also identifies residues in the interior, or core, of the protein. Such residues can also be targeted to produce proteins with improved functions, such as enhanced stability. A model of the present invention also provides additional information that is not available from other sources. For example, a model can identify the crystal contacts between crystals and predict the location of the IgE binding domain, including those amino acids that actually form contacts with a Fc domain of an IgE antibody, such as those in the binding face of the FcεRIα protein. A model can also identify the amino acids in the interface between domain 1 and domain 2 (i.e., the D1D2 interface), as well as those in the cleft formed between the two domains.

One embodiment of the present invention is a model that represents a protein that binds to a Fc domain of an IgE antibody with an affinity that is at least equivalent to the affinity of the extracellular domain of human FcεRIα for any one of the following IgE antibodies: a human IgE antibody, a canine IgE antibody, a feline IgE antibody, an equine IgE antibody, a rat IgE antibody, and a murine IgE antibody. Such a model can represent an extracellular domain of a human FcεRIα protein, a canine FcεRIα protein, a feline FcεRIα protein, an equine FcεRIα protein, a murine FcεRIα protein, and a rat FcεRIα protein. Such a model can also represent a protein with altered substrate specificity, preferably designed based on a model of the present invention. WO 98/23964, ibid., reports the ability of an isolated human FcεRIα protein to bind to canine, feline and equine IgE antibodies. Models of the present invention can be used to design a FcR with increased affinity for an antibody of a species other than self, such as, but not limited to, a human FcεRIα with increased affinity for a canine, feline or equine IgE antibody.

The present invention includes a model that represents a FcR that binds to an antibody of its respective class (i.e., IgE, IgG, IgM, IgA or IgD antibody class). Also included is a model that represents a FcR designed to bind to an antibody of a class other than the class to which the protein naturally binds. Such a model of the present invention can be produced, for example, by incorporating all or any part of the amino acid sequence of the other FcR into a 3-D model of the extracellular domain of a human FcεRIα protein. Such an embodiment includes any model that specifically incorporates any Ig domains that are placed in an orientation (packing interfaces and bend angles) that is based on the structure of the FcεRIα. A preferred model of the present invention represents a FcR that binds to an IgE antibody or to an IgG antibody. In one embodiment, a model of the present invention is a 3-D model of an extracellular antibody binding domain of a FcR other than human FcεRIα, such as of a FcR that binds to an IgG antibody. Such proteins and models thereof can be designed by homology modeling by, for example, altering the substrate specificity of a FcεRIα protein such that the altered protein binds an IgG antibody.

A preferred modified model of the present invention is a model that has a 3-D structure comprising atomic coordinates that have a root mean square deviation of protein backbone atoms of less than 10 angstrom when superimposed, using backbone atoms, on the 3-D model substantially represented by the atomic coordinates specified in Table 1, Table 5, Table 6, Table 7 or Table 8, and more particularly atomic coordinates specified in Table 1. Preferably such a model has a 3-D structure comprising atomic coordinates that have a root mean square deviation of protein backbone atoms of less than 8 angstroms, preferably less than 7 angstroms, preferably less than 6 angstroms, preferably less than 5 angstroms, preferably less than 4 angstroms, preferably less than 3 angstroms, preferably less than 2 angstroms, and preferably less than 1 angstroms, when superimposed, using backbon3 atoms, on the 3-D model substantially represented by the atomic coordinates specified in Table 1, Table 5, Table 6, Table 7, or Table 8, and more particularly atomic coordinates specified in Table 1. In this embodiment, such a model represents a FcR that binds to an antibody. The backbone atoms are those atoms that form the backbone, or 3-D folding pattern, of the model. As such, backbone atoms are the base residues of amino acids, i.e., nitrogen, carbon, the alpha carbon and oxygen. Also preferred is a model modification having an amino acid sequence that shares at least about 30%, preferably at least about 40%, more preferably at least about 45%, more preferably at least about 50%, more preferably at least about 60% and even more preferably at least about 80% amino acid sequence homology, with a human FcεRIα protein, as determined using the program ALIGN with default parameters, optimal global alignment of two sequences with no short-cuts. It is to be noted that, using the same program and parameters, the extracellular domain of a human FcεRIα protein (i.e., soluble human FcεRIα protein) shares about 48% identity with feline and rat soluble FcεRIα proteins, about 49% with a murine soluble FcεRIα protein, about 50% identity with a canine soluble FcεRIα protein, and about 60% identity with an equine soluble FcεRIα protein. A preferred model of the present invention represents an IgE binding domain, i.e., a region that binds to an IgE antibody.

One embodiment of the present invention is a 3-D model of a human FcεRIα protein produced by a method that includes the steps of: (a) crystallizing an extracellular domain of a human FcεRIα protein, such as, but not limited to a protein having amino acid sequence SEQ ID NO: 2 or SEQ ID NO: 4; (b) collecting X-ray diffraction data from the crystallized protein; and (c) determining the model from the X-ray diffraction data, preferably in combination with an amino acid sequence of the protein. A protein for crystal formation can be produced using a variety of techniques well known to those skilled in the art. As disclosed herein, a human FcεRIα protein to be crystallized is preferably produced in recombinant insect cells transformed with a gene encoding an extracellular domain of a human FcεRIα protein, such as a baculovirus genetically engineered to produce the protein. The purity of the FcεRIα protein must be sufficient to permit the production of crystals that can be analyzed by X-ray crystallography to a resolution that permits determination of a 3-D model of the protein. Preferably the resolution is at least about 4 angstroms (i.e., 4 angstroms or better), more preferably at least about 3.5 angstroms, more preferably at least about 3 angstroms, more preferably at least about 2.5 angstroms, more preferably at least about 2 angstroms and even more preferably at least about 1.5 angstroms. Methods to obtain such purity levels are well known to those skilled in the art.

As disclosed herein, a preferred method to crystallize a FcεRIα protein is by vapor distillation. Particularly preferred methods are disclosed in the Examples. It should be appreciated that the present invention also includes other methods known to those skilled in the art by which the protein can be crystallized.

3-D models of some proteins have been determined; see, for example, Blundell et al., Protein Crystallography, Academic Press, London, 1976. However, as discussed herein, elucidation of the crystal structure of the extracellular domain of the human FcεRIα was difficult. In one embodiment, crystal structure determination includes obtaining high-resolution data using synchrotron radiation. Such data can be collected, for example, at the Stanford Synchrotron Source Laboratory, Palo Alto, Calif., or the Advanced Photon Source at Argonne National Laboratories, Argonne, Ill. Additional locations to collect such data include, but are not limited to, Brookhaven, N.Y., and Japan. In one embodiment, diffraction data from native and heavy-atom treated crystals provide an initial image of the protein structure which is refined into an electron density map. Details regarding data collection and interpretation are provided in the Examples section.

One embodiment of the present invention is a method to produce a 3-D model of a FcεRIα protein that includes positioning amino acid representations (i.e., representing amino acids) of the protein at substantially the coordinates listed in Table 1, Table 5, Table 6, Table 7, or Table 8. That is, knowledge of the coordinates of the protein permits one skilled in the art to produce a model of the protein using those coordinates. Such a model, or any model which is essentially represented by a simple coordinate transformation of the coordinates specified in Table 1, Table 5, Table 6, Table 7, or Table 8, can be represented in a variety of methods as heretofore disclosed and is included in the present invention.

In another embodiment, a model of the present invention can be refined to obtain an improved model, which is an example of a model modification, also referred to as a modified model. Refining methods can include, but are not limited to, further data collection and analysis; data collection from frozen crystals; introduction of solvent molecules to the structure; clarification of secondary structure; and analyses of crystallized complexes between a FcR and an antibody or inhibitory compound. An additional model refinement method includes analyzing a 3-D model to predict amino acid residues that if replaced are likely to yield proteins with at least one improved function, effecting at least one such replacement, determining whether the activity of the modified protein agrees with the prediction, and refining the model as necessary. Methods to determine whether the modification agrees with prediction include producing the modified protein and performing assays with that modified protein to determine if the protein does indeed exhibit the improved function(s), such as desired activity, stability and solubility properties. Assays to measure such functions are well known in the art; examples of several such assays are disclosed herein.

Another embodiment of the present invention is a modified 3-D model that represents a FcR other than a human FcεRIα protein represented by the 3-D model the coordinates of which are listed in Table 1, Table 5, Table 6, Table 7, or Table 8. Preferably the amino acid sequence of the protein to be modeled is known. In such a case, the modified model can be produced using the technique of homology modeling, preferably by incorporating (e.g., grafting, overlaying or replacing) all or any portion of the amino acid sequence of the other FcR into the 3-D model of the human FcεRIα protein to produce the modified model which comprises the other FcR. General techniques for homology modeling, also referred to as molecular replacement, have been disclosed in, for example, Greer, 1990, Proteins: Structure, Function, and Genetics 7, 317-334; Havel et al., 1991, J. Mol. Biol. 217, 1-7; Schiffer et al., 1990, Proteins: Structure, Function, and Genetics 8, 30-43; and Lattman, 1985, Methods Enzymol 115, 55-77. However, such technology has not been applied to FcRs since, until the present invention, no 3-D model of any FcR was available. Thus, the present invention now allows the solving of the structures of a number of other natural and mutated forms of FcRs or any other protein with significant amino acid homology, especially to the functional Ig domains of the human FcεRIα protein.

In one embodiment, a model of a FcR, such as, but not limited to a FcεRIα protein, is produced by extracting the 3-D coordinates from a published figure or building a 3-D model with atoms from other Ig domains wherein the Ig domains are oriented as predicted for a human FcεRIα₁₋₁₇₆ protein or a FcεRIα₁₋₁₇₂ protein. For example, a model of the present invention can be produced by orienting two known Ig domains into a bent confirmation similar to that of the two domains of the human FcεRIα protein. Such a model is referred to as a model in which domain 1 and domain 2 are oriented in a manner as specified by the structural coordinates listed in Table 1, Table 5, Table 6, Table 7, or Table 8. This model can then be used in further molecular replacement methods. Such methods can include the steps of (a) orienting the model by three rotations; and (b) translating the model in one to three directions to produce additional model modifications.

Suitable FcRs for which a 3-D model can be determined using homology modeling include any mammalian FcR, such as a protein that binds to IgE, IgG, IgM, IgA or IgD antibodies. Preferred is a protein that binds to an IgE antibody or an IgG antibody. Preferred FcRs that bind to IgE include human, canine, feline, equine, murine and rat FcεRIα proteins. The present invention also includes the use of other Ig domains to produce models of the present invention.

One embodiment of the present invention is a 3-D model of a FcR having an improved function compared to an unmodified protein as well as a method to produce such a modified model. Such an improved function includes, but is not limited to, enhanced activity, enhanced stability and enhanced solubility. Such a modified model can be produced by replacing at least one amino acid based on information derived from analyzing the 3-D model of a FcεRIα protein, such as the model of a human FcεRIα₁₋₁₇₆ protein or a FcεRIα₁₋₁₇₂ protein, such that the replacement leads to a protein with an improved function. As used herein, a replacement refers to an (i.e., one or more) amino acid substitution, insertion, deletion, inversion and/or derivatization (e.g., acetylation, glycosylation, phosphorylation, PEG modification, biotinylation, and covalent attachment of other ligands or other compounds to the protein. In one embodiment, synthetic chemical methods are used to produce either a fragment or the entire protein to, for example, introduce non-natural amino acids or other chemical compounds into the structure of a FcR. For example, based on a structure of the present invention, one can design synthetic peptides or larger proteins that could be linked to produce an intact protein with IgE binding activity, the structure allowing one to design the start and stop points for these peptides, e.g., at surface accessible loops. In accordance with the present invention, an amino acid that is substituted or inserted can be a natural amino acid or an unnatural amino acid, including a derivitized amino acid. Methods to identify regions in the protein that, if changed, yield a protein with an improved function are disclosed below.

The present invention includes use of a 3-D model of the present invention to identify a compound that inhibits binding between a FcR and an antibody. The advantages of using a 3-D model to identify inhibitory compounds are multi-fold in that the model depicts the site at which a Fc domain of an antibody binds to its FcR, i.e., the antibody-binding domain, also referred to as the antibody binding site. As such, a large number of potential inhibitory compounds can be initially analyzed without having to perform in vitro or in vivo laboratory studies. As used herein, methods to identify inhibitory compounds include, but are not limited to, designing inhibitory compounds based on the 3-D model of a FcR, probing such a 3-D model with compounds that are potential inhibitors in order to identify those compounds that are actually inhibitory of the binding of an antibody to its FcR, screening a compound data base using such a 3-D model to identify compounds that inhibit such binding, and combinations thereof. Methods to use 3-D models to design, probe for, or screen for suitable inhibitory compounds are known to those skilled in the art. In particular, there are a number of computer programs that enable such methods. See, for example, PCT Publication No. WO 95/35367, by Wilson et al., published Dec. 28, 1995, which is incorporated by reference herein in its entirety.

An inhibitory compound can be any natural or synthetic compound that inhibits the binding of an antibody to a FcR. Examples include, but are not limited to, inorganic compounds, oligonucleotides, proteins, peptides, antibodies, antibody fragments, mimetics of peptides or antibodies (such as, mimetics of antibody or receptor binding sites), and other organic compounds. Compounds can inhibit binding in either a competitive or non-competitive manner and can either interact at the binding site or allosterically. An inhibitory compound should be capable of physically and structurally associating with a FcR and/or an antibody such that the compound can inhibit binding between the two entitites. As such, an inhibitory compound is preferably small and is of a structure that effectively prevents or disrupts binding. Inhibitory compounds can be identified in one or multiple steps. For example, a compound initially identified that inhibits binding between an antibody and FcR to some extent can be used as a lead to design, probe or screen for a compound with improved characteristics, such as greater efficacy, safety, solubility, etc. A preferred inhibitory compound is a compound that is efficacious when administered to an animal in an amount that results in a serum concentration of from about 1 nanomolar (nM) to 100 micromolar (μM), with a concentration of from about 10 nM to 10 μM being more preferred.

One embodiment of the present invention is a method to identify a compound that inhibits the binding between an IgE antibody and a FcεRIα protein. Such a method includes the step of using a 3-D model substantially representing the atomic coordinates specified in Table 1, Table 5, Table 6, Table 7, or Table 8 to identify such a compound. Included in the present invention are inhibitory compounds that interact directly with the IgE binding domain or the receptor binding domain of the IgE antibody as well as compounds that interact indirectly with an FcεRIα protein, such as compounds that interact with the D1D2 interface, with the cleft between D1 and D2, with a region not consisting of a N-linked glycosylation site, with a region suggested by a combination of 3-D model and mutagenesis analysis to indirectly affect antibody binding, a region suggested by homology with other FcεRIα proteins of other species, a region suggested by homology with other FcRs. In a preferred embodiment, an inhibitory compound interacts with at least one of the following regions of a model representing a FcεRIα protein: a A′B loop of D1, a EF loop of D1, a BC loop of D2, a C strand of D2, a CC′ loop of D2, a C′E loop of D2, a F strand of domain D2, a FG loop of D2, and a tryptophan-containing hydrophobic ridge. It is to be noted that the A′B and EF loops of D1 are immediately adjacent to the IgE binding domain in D2 and as such are predicted, for the first time, by the model to be good targets for inhibitory compounds. In a preferred embodiment, an inhibitory compound of the present invention interacts with at least one amino acid that is a crystal contact as predicted by the atomic coordinates listed in Table 1, Table 5, Table 6, Table 7 or Table 8. Inhibitory compounds of the present invention preferably interact with at least one of the following amino acid residues: amino acid 87, 110, 113, 115, 117, 118, 120, 121, 122, 123, 128, 129, 131, 149, 153, 154, 155, 156, 157, 158, and 159 of SEQ ID NO: 2 or SEQ ID NO: 4, as well as any surface residue within about 10 angstroms of any of the listed amino acids. More preferred is an inhibitory compound that interacts with at least one amino acid that is a crystal contact predicted to also be part of the IgE binding domain. Particularly preferred are amino acids 87, 117, 121, 123, 128, 159 of SEQ ID NO: 2 or SEQ ID NO: 4 as well as any surface residue within about 10 angstroms of amino acids 87, 117, 121, 123, 128, 159 of SEQ ID NO: 2 or SEQ ID NO: 4. In one embodiment, an inhibitory compound of the present invention is a peptide corresponding to at least a portion of any of the identified regions or a derivative thereof, such as a peptide mimetic or other compound that mimics that peptide. Preferred is a peptide corresponding to at least a portion of the FG loop of D2, or a derivative thereof, such as a peptide mimetic or other compound that mimics that peptide.

One embodiment of a method to identify a compound that inhibits the binding between an IgE antibody and a FcεRIα protein includes the steps of: (a) generating a model substantially representing the atomic coordinates listed in Table 1, Table 5, Table 6, Table 7, or Table 8, or a model of an IgE binding domain thereof, on a computer screen; (b) generating the spacial structure of a compound to be tested; and (c) testing to determine if the compound interacts with said IgE binding domain, wherein such an interaction indicates that the compound is capable of inhibiting the binding of an IgE antibody to a FcεRIα protein. In a preferred embodiment, step (a) includes the step of identifying one or more amino acid(s) in the IgE binding domain of the model that interact directly with the Fc domain of an IgE antibody when the Fc domain binds to the IgE binding domain. Preferably a compound to be tested will interact directly with one or more of those amino acid(s). Preferred amino acids with which an inhibitory compound should interact are disclosed herein.

The present invention also includes inhibitory compounds isolated in accordance with the methods disclosed herein. Methods to produce such compounds in quantities sufficient for use, for example, as protective agents (e.g., preventatives or therapeutics) are known to those skilled in the art. It should also be appreciated that it is within the scope of the present invention to expand the use of models of the present invention to produce models of any suitable FcRs (i.e., model modifications) and to identify compounds that inhibit the binding of antibodies to such FcRs.

The present invention also includes use of a 3-D model of the present invention to rationally design and construct modified forms of FcRs that have one or more improved functions, such as, but not limited to, increased activity, increased stability and increased solubility compared to an unmodified FcR. Muteins of the present invention include full-length proteins as well as fragments (i.e., truncated versions) of such proteins.

One embodiment of the present invention is a FcR that comprises a mutein that binds to a Fc domain of an antibody. Such a mutein has an improved function compared to a protein comprising SEQ ID NO: 2 or SEQ ID NO: 4. Examples of such an improved function include, but are not limited to, increased stability, increased affinity for an Fc domain of an antibody, altered substrate specificity, and increased solubility. Such a mutein can be produced by a method that includes the steps of: (a) analyzing a 3-D model substantially representing the atomic coordinates specified in Table 1, Table 5, Table 6, Table 7, or Table 8 to identify at least one amino acid of the protein represented by the model which if replaced by a specified amino acid would effect the improved function of the protein; and (b) replacing the identified amino acid(s) to produce a mutein having the improved function. Knowledge of the coordinates allows one to target specific residues, e.g. in the hydrophobic core or on the surface, to generate an accessible set of variants that can then be selected for a particular property, e.g. high stability, high affinity, altered substrate specificity, or other desirable properties (i.e., improved functions). Without the coordinates, one would have to analyze an extraordinarily large number of variants, e.g., on the order of ˜10¹¹ possibilities. The structure, in contrast, allows one to pick the most relevant residues for selecting a desired property by, for example, phage display or other methods. In a preferred embodiment, replacement of one or more amino acids does not substantially disrupt the 3-D structure of the protein; i.e., the modified protein, or mutein, is still capable of binding to the Fc domain of an antibody. A preferred mutein is a FcR that binds to a Fc domain of an IgE antibody, although the invention also covers muteins binding to other classes of antibodies.

In one embodiment, a mutein of the present invention has increased stability compared to its unmodified counterpart. As used herein, increased stability refers to the ability of a mutein to be more resistant, for example, to higher or lower temperature, to more acidic or basic pH, to higher or lower salt concentrations, to oxidation and/or reduction, to deamidation, to other forms of chemical degradation and to proteolytic degradation compared to unmodified FcR. Increased stability can also refer to the ability of a mutein of the present invention to be stable for a longer period of time either during storage (i.e., to have a longer shelf life) or during use (i.e., to have a longer half-life under reaction conditions) than does an unmodified protein. Muteins of the present invention can also exhibit a decreased entropy of unfolding, thereby stabilizing the proteins. Increased stability can be measured using a variety of methods known to those skilled in the art; examples include, but are not limited to, determination of melting temperature, thermal denaturation, pressure denaturation, enthalpy of unfolding, free energy of the protein, or stability in the presence of a chaotropic agents such as urea, guanidinium chloride, guanidinium thiocyanate, etc. A preferred mutein of the present invention has a melting temperature substantially higher than that of an unmodified FcR. Preferably the melting temperature of a mutein is at least about 1° C. higher, and more preferably at least about 10° C. higher than the melting temperature of the corresponding unmodified protein. Also preferred is a mutein having binding activity over a pH range that is at least about 1 pH unit higher and/or lower than the active pH range of the corresponding unmodified protein.

Another embodiment of the present invention is a mutein that exhibits increased affinity for a Fc domain of an antibody compared to its unmodified counterpart. As used herein, a mutein having increased affinity is a FcR that exhibits a higher affinity constant (K_{A}) or lower dissociation constant (K_{D}) than its unmodified counterpart. Such a higher affinity constant can be achieved by increasing the association rate (kₐ) between the mutein and the Fc domain and/or decreasing the dissociation rate (k_{d}) between the mutein and the Fc domain. A preferred mutein of the present invention has a K_{A} for a Fc domain of at least about 3×10⁹ liters/mole (M⁻¹), which is equivalent to a K_{D} of less than or equal to about 3.3×10⁻¹⁰ moles/liter (M). More preferred is a mutein having a K_{A} for a Fc domain of at least about 2×10¹⁰ M⁻¹, and even more preferably of at least about 1×10¹¹ M⁻¹. Also preferred is a mutein having a kₐ for a Fc domain of at least about 1×10⁵ liters/mole-second as well as a mutein having a k_{d} for a Fc domain of less than or equal to 3×10⁻⁵/second. More preferred is a mutein having a kₐ for a Fc domain of at least about 3×10⁵ liters/mole-second, and even more preferably of 1×10⁶ liters/mole-second. Also preferred are muteins having a k_{d} for a Fc domain of less than or equal to 1×10⁻⁵/second or even more preferably less than or equal to 3×10⁻⁴/second. A preferred Fc domain is that of an IgE antibody. Methods to measure such binding constants is well known to those skilled in the art; see, for example, Cook et al., 1997, ibid., which reports the following values for the binding of human FcεRIα protein to human IgE: kₐ₁ of 3.5 (±0.9)×10⁵ M⁻¹s⁻¹; kₐ₂ of 8.6 (±3.5)×10⁴ M⁻¹s⁻¹; k_{d1} of 1.2 (±0.1)×10⁻²s⁻¹; k_{d2} of 3.2 (±0.8)×10⁻⁵s⁻¹; K_{A1} of 2.0×10⁷ M⁻¹; K_{A2} of 2.9×10⁹ M⁻¹.

Another embodiment of the present invention is a mutein that exhibits altered substrate specificity compared to its unmodified counterpart. A mutein exhibiting altered substrate specificity is a mutein that binds with increased affinity to a Fc domain of an antibody class or antibody species of a different type than that normally bound by its unmodified counterpart. In one embodiment, a mutein of a human FcεRIα protein with altered substrate specificity is a FcR that binds with increased affinity to a IgE antibody of another mammal, such as, but not limited to, a canine, feline, equine, murine, or rat IgE antibody. In another embodiment, a mutein of a human FcεRIα protein with altered substrate specificity is a FcR that binds with increased affinity to an antibody of another class, such as IgG, IgM, IgA, or IgD, with IgG being preferred. Such a mutein can also show altered species substrate specificity. Methods to determine whether a mutein exhibits altered substrate specificity are well known to those skilled in the art.

Yet another embodiment of the present invention is a mutein that exhibits increased solubility compared to its unmodified counterpart. Such a protein is less likely to form aggregates. Methods to determine whether a mutein exhibits increased solubility are well known to those skilled in the art.

As disclosed herein, the 3-D model representing a FcεRIα protein is advantageous in determining strategies for producing muteins having an improved function, e.g., for identifying targets to modify in order to obtain muteins having improved functions. Examples of targets are as follows. A key feature of the human FcεRIα₁₋₁₇₆ protein or the FcεRIα₁₋₁₇₂ protein is the crystal contacts in five space groups, a subset of which are predicted to interact directly with a Fc domain of an IgE antibody. Such contacts are included in the IgE binding domain which is unique for human FcεRIα in that the domain includes a tryptophan-containing hydrophobic ridge positioned on the top face of the crystal structure (i.e., amino acids W87, W110, W113, and W156 of SEQ ID NO: 2 or SEQ ID NO: 4) and an FG loop comprising amino acids from 155 to 158 of SEQ ID NO: 2 or SEQ ID NO: 4 that protrudes above the interface in an unusual manner. Another key feature is the interface between domain 1 and domain 2 (i.e., the D1D2 interface) which includes amino acids 12, 13, 14, 15, 16, 17, 18, 20, 84, 85 and 86 in D1 and 87, 88, 89, 90, 91, 92, 93, 95, 104, 106, 108, 110, 111, 161, 163, 164, and 165 in D2 of SEQ ID NO: 2 or SEQ ID NO: 4. Also important are the two domains themselves: D1 includes amino acids 1 through 86 of SEQ ID NO: 2 or SEQ ID NO: 4; and D2 includes amino acids 87 through 176 of SEQ ID NO: 2 or amino acids 87 through 172 of SEQ ID NO: 4. Another important feature is the cleft between D1 and D2, which can be identified using the coordinates. Other areas of interest include the hydrophobic core which can be identified using the coordinates, the A′B loop of D1, which includes amino acids 18 and 19, the EF loop of D1, which includes amino acids 59-63, the BC loop of D2, which includes amino acids 110-114, the C strand of D2, which includes amino acids 114-123, the CC′ loop of D2, which includes amino acids 123-125, the C′E loop of D2, which includes amino acids 127-134, in the different confirmations observed in the five crystal forms, and the F strand of D2, which includes amino acids 147-155 of SEQ ID NO: 2 or SEQ ID NO: 4. Yet another striking feature is the finding that the amino and carboxyl termini of the human FcεRIα₁₋₁₇₆ protein are only 10 angstroms apart.

In accordance with the present invention, a mutein having an improved function can be produced by a method that includes replacing at least one amino acid based on information derived from analyzing a 3-D model of the present invention to produce the mutein having the improved function. Knowledge of the structure of the extracellular domain of a human FcεRIα protein crystal, for example, permits the rational design and construction of modified forms of the protein by permitting the prediction and production of substitutions, insertions, deletions, inversions and/or derivatizations that effect an improved function. That is, analysis of 3-D models of the present invention provide information as to which amino acid residues are important and, as such, which amino acids can be changed without harming the protein. In making amino acid replacements, it is preferred to use amino acid replacements that have similar numbers of atoms and that allow conservation of salt bridges, hydrophobic interactions and hydrogen bonds unless the goal is to purposefully change such interactions. The 3-D structure of the human FcεRIα protein suggests that large deletions may not be desirable, particularly due to the relation between the various domains of the protein and the observation that most of the structure is well ordered in the crystal. An exception to this is the non-constrained loops of D1, which apparently could be deleted or shortened without harming the protein's function. These loops span amino acids 31-35 and 70-74 of SEQ ID NO: 2 or SEQ ID NO: 4.

It is to be appreciated that although one amino acid replacement capable of improving the function of a protein can substantially improve that function, more than one amino acid replacement can result in cumulative changes depending on the number and location of the replacements. For example, although one amino acid replacement capable of substantially increasing the stability of a protein can increase the melting temperature of that modified protein by about 1° C., about 5 to about 6 replacements may increase the melting temperature of the resultant protein by about 10° C.

In accordance with the present invention, the 3-D model of the human FcεRIα protein has been analyzed, using techniques known to those skilled in the art, to determine the accessibility of the amino acids represented within the model to solvent. Such information is provided in, for example, Table 2, Table 9, Table 10, Table 11, and Table 12.

A number of methods can be used to produce muteins of the present invention. One method includes the steps of: (a) analyzing a 3-D model substantially representing the coordinates specified in Table 1, Table 5, Table 6, Table 7, or Table 8 to identify at least one amino acid of the modeled protein which if replaced by a specified amino acid would effect an improved function; and (b) replacing the identified amino acid(s) to produce a mutein having that improved function. In one embodiment, a method to produce a mutein includes the steps of (a) comparing a key region of a model of a human FcεRIα protein with the amino acid sequence of a FcR having an improved function compared to the unmodified FcεRIα protein in order to identify at least one amino acid segment of the FcR with the improved function that if incorporated into the FcεRIα protein represented by the model would give the FcεRIα protein the improved function; and (b) incorporating the segment into the FcεRIα protein, thereby providing a mutein with the improved function. In another embodiment, a method to produce a protein includes the steps of: (a) using a model representing a human FcεRIα protein to identify a 3-D arrangement of residues that can be randomized by mutagenesis to allow the construction of a library of molecules from which a improved function can be selected; and (b) identifying at least one member of the mutagenized library having the improved function. In one example, a mutein is produced by a method that includes the steps of: (a) effecting random mutagenesis of nucleic acid molecules encoding a target of a FcεRIα protein as identified by analyzing a model of that protein, such as an IgE binding domain; (b) cloning such mutagenized nucleic acid molecules into a phage display library, wherein said phage display library expresses the target; and (c) identifying at least one member of the library that expresses a target with an improved function, such as an antibody binding domain exhibiting increased affinity for an antibody. As stated above, the model allows the use of this technique in a straightforward manner that could not be accomplished in the absence of the model. It is to be also noted that these methods can also be used with other models of the present invention to produce muteins of the present invention.

The present invention includes a number of methods, based on analysis of a 3-D model of the present invention, to replace (i.e., add, delete, substitute, invert, derivatize) at least one amino acid residue in the protein represented by the model in order to produce a mutein of the present invention. Such methods include, but are not limited to: (a) replacing at least one amino acid in at least one non-constrained loop of domain 1 in an area proximal to the FceRI gamma chain putative binding site; (b) joining an amino-terminal amino acid residue to a carboxyl-terminal amino acid residue of an extracellular domain of a FcεRIα protein; (c) replacing at least one amino acid site with an amino acid suitable for derivatization; (d) replacing at least one pair of amino acids of the protein with a cysteine pair to enable the formation of a disulfide bond that stabilizes the protein; (e) removing at least a portion of the region between the B strand and C strand of domain 1; (f) removing at least a portion of the region between the C strand and E strand of domain 1; (g) replacing at least one amino acid in the IgE binding domain in order to increase the affinity between an IgE antibody and the protein; (h) replacing at least one amino acid of the protein with an amino acid such that the replacement decreases the entropy of unfolding of the protein; (i) replacing at least one asparagine or glutamine of the protein with an amino acid that is less susceptible to deamidation than is the amino acid to be replaced; (j) replacing at least one methionine, histidine or tryptophan with an amino acid that is less susceptible to an oxidation or reduction reaction than is the amino acid to be replaced; (k) replacing at least one arginine of the protein with an amino acid that is less susceptible to dicarbonyl compound modification than is the amino acid to be replaced; (l) replacing at least one amino acid of the protein susceptible to reaction with a reducing sugar sufficient to reduce protein function with an amino acid less susceptible to that reaction; (m) replacing at least one amino acid of the protein with an amino acid capable of increasing the stability of the inner core of the protein; (n) replacing at least one amino acid of the protein with at least one N-linked glycosylation site; (o) replacing at least one N-linked glycosylation site of the protein with at least one amino acid that does not comprise an N-linked glycosylation site; and (p) replacing at least one amino acid of the protein with an amino acid that reduces aggregation of the protein.

Amino acid replacements can be carried out using recombinant DNA techniques known to those skilled in the art, including site-directed mutagenesis (e.g., oligonucleotide mutagenesis, random mutagenesis, polymerase chain reaction (PCR)-aided mutagenesis, gapped-circle site-directed mutagenesis) or chemical synthetic methods of a nucleic acid molecule encoding the desired protein, such as, but not limited to a human FcεRIα protein, followed by expression of the mutated gene in a suitable expression system, preferably an insect, mammalian, bacterial, yeast, insect, or mammalian expression system. See, for example, Sambrook et al., ibid.

One embodiment of the present invention is a mutein in which at least one amino acid in at least one non-constrained loop of a FcεRIα protein is replaced in order to improve a function of the protein. Finding that the human FcεRIα protein had such loops was surprising, and it is believed, without being bound by theory, that a mutein in which at least a portion of at least one such loop is replaced, would at least exhibit enhanced stability. In a preferred embodiment, at least a portion of one or more of such loops is (are) deleted. Preferred loops to replace are in domain 1 (i.e., spanning amino acids 31-35 and 70-74 of SEQ ID NO: 2 or SEQ ID NO: 4), preferably in an area proximal to the FceRI gamma chain putative binding site, i.e., the site on the FcεRIα protein to which the gamma chain of the high affinity Fc epsilon receptor is thought to bind. In a preferred embodiment, one or more amino acids is replaced to make loops shorter, but including 1 or 2 hydrophobic residues to pack toward the protein interior and at least one hydrophilic residue to maintain solubility.

Another embodiment of the present invention is a mutein of the extracellular domain of a FcεRIα protein in which an N-terminal (amino-terminal) amino acid residue is joined, preferably covalently, to a C-terminal (carboxyl-terminal) amino acid residue in order to improve a function of the protein. Finding that the N-termini and C-termini of the human FcεRIα protein were only 10 angstroms apart was quite surprising. Without being bound by theory, it is believed that such a mutein would at least exhibit enhanced stability. Furthermore, a covalent linker used to join the termini could also include a substance useful, for example, to anchor a mutein on a surface, as would be useful, for example, in a diagnostic assay, or to label the mutein. For a protein consisting of SEQ ID NO: 2, a preferred N-terminal residue is an amino acid residue at position 1, 2, or 3 of SEQ ID NO: 2, and a preferred C-terminal residue is an amino acid residue at position 174, 175, or 176 of SEQ ID NO: 2. Covalent linkage can be accomplished by methods known to those skilled in the art, such as, but not limited to, adding one or more N-terminal and C-terminal cysteines and crosslinking them with chemical compounds, adding additional residues in the coding sequence to allow the formation of a disulfide bond, or adding one or more lysines and coupling them through a 10 angstrom linker, and including non-natural amino acid analogues by synthetic methods or by a combination of biosynthetic and organosynthetic methods. Examples of a substance to add to a covalent linker includes: ligands useful in allowing for the attachment of a mutein to a surface, such as biotin and related compounds, avidin and related compounds, metal binding compounds, sugar binding compounds, immunoglobulin binding domains, and other tag domains; and detectable markers, such as enzyme labels, physical labels, radioactive labels, fluorescent labels, chemiluminescent labels, and chromophoric labels. Examples include, but are not limited to, alkaline phosphatase, horseradish peroxidase, digoxygenin, luciferase, other light-generating enzymes and magnetic beads. It is also to be noted that ligands can function as detectable markers.

Another embodiment of the present invention is a mutein in which at least one amino acid is replaced with an amino acid suitable for derivatization. Muteins in which at least one amino acid is replaced with an amino acid suitable for derivatization include proteins that are chemically modified (e.g., a lysine already existing on the protein is modified) as well as those in which an amino acid residue is replaced with a different amino acid residue (e.g., a glycine with a lysine) as well as proteins to which a substance is added, preferably to the amino or carboxyl terminus of the protein. Examples of such substances include ligands and detectable markers as disclosed above. Preferable amino acids to replace include residues that are solvent exposed (e.g., those listed in Table 2, Table 9, Table 10, Table 11, or Table 12), but that are preferably not within about 10 angstroms of the IgE binding domain. In one embodiment, a glycosylation site, or other solvent exposed site, is replaced with a charged or polar residue to increase solubility or create more stable muteins. Glycosylation sites in human FcεRIα protein include amino acids 21, 42, 50 74, 135, 140, and 166 of SEQ ID NO: 2 or SEQ ID NO: 4. A preferred amino acid to use as a replacement, or to chemically modify directly, includes a cysteine or a lysine, with a cysteine being preferred. Compounds to use in chemical derivatizations are known to those skilled in the art; cysteines can, for example, be derivatized with maleimides.

Another embodiment of the present invention is a mutein in which a pair of amino acids have been replaced with a cysteine pair in order to improve the function of the mutein, at least by increasing stability. Cysteine pairs can be substituted into a FcεRIα protein at any two residue positions identified with available programs and algorithms that would allow the formation of an undistorted disulfide bridge. In one embodiment, a serine and lysine near the termini of the protein is each replaced with a cysteine. In another embodiment, cysteine pairs are replaced with other amino acids, such as serines to eliminate non-essential disulfide bonds.

Another embodiment of the present invention is a mutein in which at least one amino acid is replaced in the region between the B strand and C strand of domain 1 and/or the region between the C and E strand of domain 1. In a preferred embodiment, at least a portion of such a region is deleted.

Another embodiment of the present invention is a mutein in which at least one amino acid is replaced in the IgE binding domain in order to increase the affinity between an IgE antibody and the protein. Preferred residues to replace are in or near the IgE binding domain, or IgE binding site, as determined by analysis of the 3-D model. Such residues are preferably within about 10 angstroms of residues identified by mutagenesis and further shown by model to be in an IgE binding site. Examples of such residues include amino acids 87, 110, 113, 115, 117, 118, 120, 121, 122, 123, 128, 129, 131, 149, 153, 154, 155, 156, 157, 158, and 159 of SEQ ID NO: 2 or SEQ ID NO: 4, and amino acids within 10 angstroms of such listed amino acids. In one embodiment, preferred amino acids to replace include amino acids 87, 115, 117, 118, 120-123, 128, 129, 131, 149, 153, 155 and 159 of SEQ ID NO: 2 or SEQ ID NO: 4 as well as any surface residue within about 10 angstroms of any of the listed amino acids, with amino acids 87, 117, 121, 123, 128, 159 of SEQ ID NO: 2 or SEQ ID NO: 4 as well as any surface residue within about 10 angstroms of amino acids 87, 117, 121, 123, 128, 159 of SEQ ID NO: 2 or SEQ ID NO: 4 being particularly preferred. It is to be noted that amino acids 115, 118, 120, 131, 149 and 155 of SEQ ID NO: 2 or SEQ ID NO: 4 are buried, and that amino acids that are partially buried or glycine include residues 122, 129 and 153. Additional amino acid residues to target include those in the A′B loop of D1, and EF loop of D1. Note that these residues are not the same as those shown in mutation studies to affect IgE binding since some of those mutants have mutations in amino acids that are internal to the protein; this finding can only be made by analysis of a model of the present invention.

Another embodiment of the present invention is a mutein in which at least one amino acid is replaced with an amino acid capable of increasing the stability of the inner core or surface of the protein. Preferred amino acids to replace are hydrophilic residues located in the hydrophobic core of the protein and/or hydrophobic amino acids at the protein surface that are not within about 10 angstroms of the IgE binding domain residues of D1 or D2. Preferred amino acids to replace into the hydrophobic core are hydrophobic residues such as, but not limited to, tryptophan, leucine, isoleucine, valine and alanine, as well as space filling amino acids, such as other aromatic amino acids. Preferred amino acids to replace onto the surface are polar amino acids, such as, but not limited to, glutamic acid, glutamine, aspartic acid, asparagine, histidine and serine. Muteins having one or more such amino acid replacements would exhibit at least increased stability and/or reduced aggregation. Additional preferred amino acid replacements are those that introduce salt bridges at the protein surface to stabilize protein folds. It is noted that the cysteines at positions 26 and 68 of SEQ ID NO: 2 or SEQ ID NO: 4 form a disulfide bond in domain 1 that is somewhat exposed to solvent, depending especially on the conformation of the D1 “30 loop” (i.e., amino acids 31-35 of SEQ ID NO: 2 or SEQ ID NO: 4). In one embodiment, changes in neighboring residues can be made in, for example, residues 1-5, 27-37, 49-52, or 69-75, to bury this disulfide from exposure to solvent. For example, phage display of receptors with randomized mutations in the 30 loop, might be useful for selecting receptors that react less well with reducing reagents and have a more stable D1 core.

Another embodiment of the present invention is a mutein in which at least one amino acid is replaced with an amino acid that decreases the entropy of unfolding of the protein. The entropy of unfolding of a protein can be measured and compared to that of another protein using techniques known to those skilled in the art. A number of methods known to those skilled in the art can be used to reduce the number of protein conformations possible in the unfolded state, thereby improving the ability of the protein to fold correctly. One embodiment of the present invention for decreasing the entropy of unfolding includes replacing at least one amino acid of the protein with a specified amino acid in order to maintain certain desirable phi and psi backbone conformation angles in the protein; see, for example, PCT International Publication No. WO 89/01520, by Drummond et al., published Feb. 23, 1989. For example, a proline residue in a protein constrains the backbone conformation to certain restricted angles. Analysis of a 3-D model of a protein of the present invention permits the identification of candidate replacement positions in the protein that have the conformation expected for a proline, but that do not have a proline in them. Such knowledge is used to introduce prolines into such candidate replacement positions to “anchor” the resultant mutein in the desired conformation. The 3-D model also permits the identification of candidate replacement sites that if replaced with a proline do not substantially disrupt the 3-D structure of the resultant protein. Similarly, glycines in appropriate positions can be replaced with an amino acid having a β carbon atom or a branched β carbon atom, preferably an alanine, in order to stabilize the backbone of the protein.

Another embodiment of the present invention is a mutein in which at least one asparagine or glutamine is replaced with an amino acid that is less susceptible to . deamidation. Preferred amino acids to replace include solvent accessible asparagines and glutamines.

Another embodiment of the present invention is a mutein in which at least one methionine, histidine or tryptophan is replaced with an amino acid that is less susceptible to an oxidation or reduction reaction. Preferred amino acids to replace include M98, H70, and H41. It would not be preferred to replace any of the tryptophans, nor H108 or H134 of SEQ ID NO: 2 or SEQ ID NO: 4.

Another embodiment of the present invention is a mutein in which at least one arginine is replaced with an amino acid that is less susceptible to dicarbonyl compound modification. Although R174 could be changed, it would probably not be preferable to change amino acids at the D1D2 interface or near the IgE binding site, such as amino acids 15, 106, or 111 of SEQ ID NO: 2.

Another embodiment of the present invention is a mutein in which at least one amino acid that is susceptible to reaction with a reducing sugar sufficient to reduce protein function is replaced with an amino acid that is less susceptible to such a reaction. For example, lysines, glutamines and asparagines that could react with a sugar, such as galactose, glucose or lactose can be replaced with non-reactive amino acids.

Another embodiment of the present invention is a mutein in which one or more N-linked glycosylation sites are added to or removed from the protein, preferably by substitution with an appropriate amino acid. A FcεRIα protein with additional N-linked glycosylation sites is more soluble. The ability to design a FcεRIα protein having fewer, or no, N-linked glycosylation sites is also valuable as production of such a protein from production run to production run is likely to be more uniform. One embodiment is a FcεRIα mutein with no N-linked glycosylation sites that is stable, active, and soluble. Such a protein has an advantage of being produced in E. coli at low cost. In one embodiment, one or more exposed hydrophobic amino acids are changed to charged residues that form salt bridges to stabilize the protein fold and make it soluble. It is to be noted that the glycosylation sites that appear to be most often observed in the different crystal structures in the same conformation are the carbohydrate attached to positions 42 and 166 of SEQ ID NO: 2 or SEQ ID NO: 4. The carbohydrate attached to position 42 always appears to cover the phenylalanine at position 60 of SEQ ID NO: 2 or SEQ ID NO: 4. As such, one embodiment of the present invention is to remove the glycosylation site at position 42, e.g., by substitution with a suitable amino acid. This embodiment has the additional advantage that the resultant mutein has an exposed phenylalanine at position 60, thereby leading to increased IgE binding activity.

Another embodiment of the present invention is a mutein in which at least one amino acid is replaced with an amino acid that reduces aggregation and increases solubility of the protein, such as, for example, replacing one or more hydrophobic residues on the surface with one or more hydrophilic residues. Other examples of such amino acids to replace are disclosed herein.

Another embodiment of the present invention to enhance stability is the addition of polyethylene glycol (PEG) groups to a FcR protein, i.e., to produce a “pegylated” FcR protein. In one embodiment, the PEG group(s) can substitute for carbohydrate group(s) due to removal of one or more N-glycosylation sites. Such PEG group(s) can be attached to easily modifiable residues, such as cysteines or lysines, on the surface of the protein, such residues identifiable by analysis of a 3-D model of the present invention.

Another embodiment of the present invention is a mutein that comprises a FcR having a substance, such as a ligand or detectable marker, attached to an amino acid of the protein such that the substance does not substantially interfere with the antibody binding activity of the protein. The substance is attached in such a manner that the substance is also capable of performing its function, such as binding to a second member of a ligand pair or enabling detection of the protein. The FcR to which a substance is attached can be either an unmodified protein or a mutein of the present invention. Suitable attachment sites can be identified using 3-D models of the present invention. Preferred attachment sites include solvent exposed amino acids, such as those listed in Table 2, Table 9, Table 10, Table 11, or Table 12. Substances can be attached, or conjugated, to the protein using techniques known to those skilled in the art. It is to be appreciated that a preferred method to attach a substance to an amino acid is to modify that amino acid to have a reactive attachment site, such as is present on cysteine and lysine amino acids. As such, an attachment site comprising a solvent exposed amino acid refers to the nature of the amino acid prior to any modification required for attachment. Examples of suitable substances to attach to a FcR include any compound capable of binding to or reacting with another substance, such as those described for attachment to a covalent linker.

It is to be appreciated that muteins of the present invention can include amino acids which are not modified because they would negatively impact the function of the protein. Such amino acids can be identified using a 3-D model of the present invention.

It should also be appreciated that it is within the scope of the present invention to expand the use of models of the present invention to produce models of and make modifications to any suitable FcRs or other Ig domain-containing proteins to produce muteins having a desired function.

The present invention also includes nucleic acid molecules that encode muteins of the present invention as well as recombinant molecules and recombinant cells that include such nucleic acid molecules. Methods to produce such proteins are also disclosed herein.

The present invention includes an isolated FcεRIα protein that consists of SEQ ID NO: 2, i.e., PhFcεRIα₁₋₁₇₆. Also included in the present invention is a protein consisting of an extracellular domain of a FcεRIα protein that is structurally homologous to an isolated FcεRIα protein consisting of SEQ ID NO: 2. As used herein, a protein that is structurally homologous to PhFcεRIα₁₋₁₇₆ is a protein that (a) includes both D1 and D2 domains, (b) shares at least about 30%, and preferably at least about 40%, amino acid sequence identity with SEQ ID NO: 2, as determined using a ALIGN with default parameters, optimal global alignment of two sequences with no short-cuts, (c) displays a substantially equivalent affinity for an IgE antibody as does a complete extracellular domain of the corresponding FcεRIα protein, and (d) produces crystals having sufficient quality to enable structure determination. Examples of such proteins include a human FcεRIα protein having SEQ ID NO: 4, i.e., PhFcεRIα₁₋₁₇₂ and a human FcεRIα protein having an amino acid sequence that spans from amino acid 3 through amino acid 174 of SEQ ID NO: 2, i.e., PhFcεRIα₃₋₁₇₄. It is to be noted that these examples are provided to clarify the definition of a structurally homologous FcεRIα protein and are not intended to limit the scope of such proteins. That is, a FcεRIα protein that is structurally homologous to PhFcεRIα₁₋₁₇₆ is any mammalian FcεRIα protein having the listed characteristics. Preferred are human, canine, feline, equine, murine and rat proteins that are structurally homologous to PhFcεRIα₁₋₁₇₆. Also included herein are nucleic acid molecules to encode such proteins as well as recombinant molecules and recombinant cells that include such nucleic acid molecules. Methods to produce such proteins are also disclosed herein. Preferably such proteins are produced in insect cells.

The present invention also includes a FcεRIα protein consisting of SEQ ID NO: 4 except that the isoleucine at position 170 has been replaced by a cysteine. Also included in the present invention is a protein consisting of an extracellular domain of a FcεRIα protein that is structurally homologous to an isolated FcεRIα protein consisting of SEQ ID NO: 4 except that the isoleucine at position 170 has been replaced by a cysteine.

The present invention also includes the following novel structures as identified by a 3-D model of the present invention: a crystal contact cluster, preferably involved in IgE binding; a tryptophan-containing hydrophobic ridge; a FG loop in D2; a D1D2 interface; a cleft between D1 and D2; a domain 1; a domain 2; a hydrophobic core; a A′B loop of D1; a EF loop of D1; a BC loop of D2; a C strand of D2; a CC′ loop of D2; a C′E loop of D2; and a strand of D2. Also included herein are nucleic acid molecules to encode such structures as well as recombinant molecules and recombinant cells that include such nucleic acid molecules. Also included are methods to produce such structures and models thereof.

The present invention also includes isolated nucleic acid molecules encoding proteins of the present invention, including, but not limited to, proteins comprising unmodified extracellular domains of FcRs, novel structures within such proteins, and muteins. As used herein, an isolated nucleic acid molecule encoding a protein is a nucleic acid molecule that has been removed from its natural milieu. As such, “isolated” does not reflect the extent to which the nucleic acid molecule has been purified. An isolated nucleic acid molecule can be DNA, RNA, or derivatives of either DNA or RNA.

A nucleic acid molecule encoding a mutein of the present invention can be produced by mutation of parental protein genes (e.g., unmodified or previously modified protein-encoding genes, or portions thereof) using recombinant DNA techniques heretofore disclosed or by chemical synthesis. Resultant mutein nucleic acid molecules can be amplified using recombinant DNA techniques known to those skilled in the art, such as PCR amplification or cloning (see, for example, Sambrook et al., ibid.), or by chemical synthesis. A mutein can also be produced by chemical modification of a protein expressed by a nucleic acid molecule encoding an unmodified protein or mutein-encoding gene.

Proteins of the present invention can be produced in a variety of ways, including production and recovery of recombinant proteins and chemical synthesis. In one embodiment, a protein of the present invention is produced by culturing a cell capable of expressing the protein under conditions effective to produce the protein, and recovering the protein. A preferred cell to culture is a recombinant cell that is capable of expressing the protein, the recombinant cell being produced by transforming a host cell with one or more nucleic acid molecules of the present invention. Transformation of a nucleic acid molecule into a host cell can be accomplished by any method by which a nucleic acid molecule can be inserted into a cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of a host cell in such a manner that their ability to be expressed is retained.

Suitable host cells to transform include any cell that can be transformed. Host cells can be either untransformed cells or cells that are already transformed with at least one nucleic acid molecule. Host cells of the present invention can be endogenously (i.e., naturally) capable of producing a protein of the present invention, but such cells are not preferred. Host cells of the present invention can be any cell that when transformed with a nucleic acid molecule of the present invention are capable of producing a protein of the present invention, including bacterial, yeast, other fungal, insect, animal, and plant cells. Preferred host cells include bacterial, yeast, insect and mammalian cells, and more preferred host cells include Escherichia, Bacillus, Saccharomyces, Pichia, Trichoplusia, Spodoptera and mammalian cells. Particularly preferred host cells are Trichoplusia ni cells, Spodoptera frugiperda cells, and Chinese hamster ovary cells.

A recombinant cell is preferably produced by transforming a host cell with a recombinant molecule comprising a nucleic acid molecule of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell, of replicating within the host cell, and of effecting expression of a specified nucleic acid molecule. Expression vectors can be either prokaryotic or eukaryotic, and are typically viruses or plasmids. Expression vectors of the present invention include any vectors that function (i.e., direct gene expression) in recombinant cells of the present invention, including in bacterial, yeast, other fungal, insect, animal, and plant cells. Preferred expression vectors of the present invention can direct gene expression in bacterial, yeast, insect and mammalian cells.

Nucleic acid molecules of the present invention can be operatively linked to expression vectors containing regulatory control sequences such as promoters, operators, repressors, enhancers, termination sequences, origins of replication, and other regulatory control sequences that are compatible with the host cell and that control the expression of the nucleic acid molecules. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, insect and mammalian cells.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant protein production during fermentation. The activity of an expressed recombinant protein of the present invention may be improved by fragmenting, modifying, or derivatizing nucleic acid molecules encoding such a protein.

In accordance with the present invention, recombinant cells can be used to produce proteins by culturing such cells under conditions effective to produce such a protein, and recovering the protein. Effective conditions to produce a protein include, but are not limited to, appropriate media, bioreactor, temperature, pH and oxygen conditions that permit protein production. An appropriate medium refers to any medium in which a cell of the present invention, when cultured, is capable of producing the protein. An effective medium is typically an aqueous medium comprising assimilable carbohydrate, nitrogen and phosphate sources, as well as appropriate salts, minerals, metals and other nutrients, such as vitamins. The medium may comprise complex nutrients or may be a defined minimal medium. Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant proteins may either remain within the recombinant cell; be secreted into the fermentation medium; be secreted into a space between two cellular membranes, such as the periplasmic space in E. coli; or be retained on the outer surface of a cell or viral membrane. The phrase “recovering the protein” refers simply to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization.

The present invention also includes isolated (i.e., removed from their natural milieu) antibodies that selectively bind to a FcR of the present invention (i.e., anti-FcR antibodies). As used herein, the term “selectively binds to” FcR refers to the ability of antibodies of the present invention to preferentially bind to specified proteins of the present invention. Binding can be measured using a variety of methods standard in the art including enzyme immunoassays (e.g., ELISA), immunoblot assays, etc.; see, for example, Sambrook et al., ibid. Isolated antibodies of the present invention can include antibodies in a bodily fluid (such as, but not limited to, serum), or antibodies that have been purified to varying degrees. Antibodies of the present invention can be polyclonal or monoclonal. Functional equivalents of such antibodies, such as antibody fragments and genetically-engineered antibodies (including single chain antibodies or chimeric antibodies that can bind to more than one epitope) are also included in the present invention. Antibodies can be produced using methods known to those skilled in the art. A preferred method to produce antibodies of the present invention includes (a) administering to an animal an effective amount of a protein of the present invention to produce the antibodies and (b) recovering the antibodies. In another method, antibodies of the present invention are produced recombinantly using techniques as heretofore disclosed to produce proteins of the present invention. Antibodies raised against defined proteins can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. Examples of such uses are disclosed in WO 98/27208, ibid., see, for example, page 24; such uses are incorporated by reference herein in their entireties.

A FcR of the present invention can include chimeric molecules comprising at least a portion of a FcR that binds to an antibody and a second molecule that enables the chimeric molecule to be bound to a substrate in such a manner that the antibody receptor portion binds to the antibody in at least as effective a manner as a FcR that is not bound to a substrate. An example of a suitable second molecule includes a portion of an immunoglobulin molecule or another ligand that has a suitable binding partner that can be immobilized on a substrate, e.g., biotin and avidin, or a metal-binding protein and a metal (e.g., His), or a sugar-binding protein and a sugar (e.g., maltose).

The present invention includes uses of proteins, antibodies and inhibitory compounds of the present invention for the diagnosis and treatment of allergy and the regulation of other immune responses in an animal.

One embodiment is a therapeutic composition comprising at least one of the following therapeutic compounds: an inhibitory compound of the present invention, a mutein of the present invention, or an antibody of the present invention. Also included is a method to protect an animal from allergy or other abnormal immune responses. Such a method includes the step of administering a therapeutic composition of the present invention to the animal. As used herein, the ability of a therapeutic composition of the present invention to protect an animal from allergy or other abnormal immune responses refers to the ability of that composition to, for example, treat, ameliorate or prevent allergy or other abnormal immune responses. General characteristics of therapeutic compositions and methods to produce and use such therapeutic compositions are disclosed, for example, in WO 98/27208, ibid., see, for example, page 39-47; such compositions and methods are incorporated by reference herein in their entireties. It is to be noted that although the compositions and methods disclosed in WO 98/27208, ibid., relate to feline FcεRIα proteins, they are also applicable to therapeutic compositions of the present invention. Therapeutic compositions of the present invention are advantageous because they can be derived from analysis of 3-D models of the present invention and have improved functions, such as efficacy and safety.

Another embodiment is a diagnostic reagent comprising a mutein of the present invention. As used herein, a diagnostic reagent is a composition that includes a mutein that is used to detect allergy or other abnormal immune responses in an animal. Also included in the present invention are methods, including in vivo methods and in vitro methods, to (a) detect allergy or other abnormal immune response, or susceptibility thereto, in an animal, comprising use of a diagnostic reagent comprising a mutein of the present invention and (b) to enhance the performance of an IgE binding assay, said method comprising incorporating into the assay a mutein of the present invention. General characteristics of diagnostic reagents and methods to produce and use such diagnostic reagents are disclosed, for example, in WO 98/27208, ibid., see, for example, page 2-39; such reagents and methods are incorporated by reference herein in their entireties. It is to be noted that although the reagents and methods disclosed in WO 98/27208, ibid., relate to feline FcεRIα proteins, they are also applicable to diagnostic reagents, kits and detection methods of the present invention. Muteins of the present invention are advantageous in such applications because of their enhanced affinity for antibodies, altered specificity, enhanced solubility and/or enhanced stability, enabling for example use in otherwise adverse conditions and longer shelf-life.

The following examples are provided for the purposes of illustration and are not intended to limit the scope of the invention.

### EXAMPLES

### Example 1

This Example describes the production of a FcεRIα nucleic acid molecule, a recombinant molecule, a recombinant cell, a recombinant virus, and a FcεRIα protein of the present invention.

A number of human FcεRIα proteins of variable lengths (i.e., 171, 172, and 176 amino acids) were produced in a variety of cell lines (i.e., Chinese hamster ovary cells, Pichia pastoris yeast, Spodoptera frugiperda (Sf9) insect cells and Trichoplusia ni (Hi-5) insect cells). Due to a number of factors, however, including protein length, solubility, and extent and variability of glycosylation, only one FcεRIα protein was useful in producing a crystal of sufficient quality for the first determination of a model of an extracellular domain of a FcεRIα protein. The production of this protein is disclosed below.

A nucleic acid molecule comprising the first 176 amino acids of the mature form of the human FcεRIα protein, nucleic acid molecule and protein designated herein as nhFcεRIα₁₋₅₂₈ and PhFcεRIα₁₋₁₇₆, respectively, was produced as follows. An EcoRI-HindIII fragment from plasmid EdpC20 (Blank et al., ibid.) containing the human FcεRIα signal sequence and residues 1-172 of the mature human FcεRIα protein was ligated to two oligonucleotides coding for residues 172-176 of the mature protein and two stop codons. The two oligonucleotides, having nucleic acid sequences of 540 AGCTCCGCGT GAGAAGTAAT AAG 3′ (SEQ ID NO: 5) and 5′GATCCTTATT ACTTCTCACG CGG 3′ (SEQ ID NO: 6), had HindIII and BamHI overhangs when annealed together, which permitted the ligation of nhFcεRIα₁₋₅₂₈ into EcoRI and HindIII cleaved baculovirus transfer vector pVL1392 (available from Pharmingen, San Diego, Calif.) to produce recombinant molecule pVL1392-nhFcεRIα₁₋₅₂₈. The resultant construct was verified by DNA sequencing.

Recombinant virus was produced as follows. Recombinant molecule pVL1392-nhFcεRIα₁₋₅₂₈ was co-transfected with a linear Baculogold baculovirus DNA (available from Pharmingen) into S. frugiperda Sf9 cells to form recombinant cell Sf9:pVL1392-nhFcεRIα₁₋₅₂₈ which was cultured to produce recombinant virus, namely BV:pVL1392-nhFcεRIα₁₋₅₂₈ using techniques known to those skilled in the art. Supernatants of transfected Sf9:pVL1392-nhFcεRIα₁₋₅₂₈ cells were amplified once in TNM-FH medium (available from Pharmingen), followed by a second amplification in serum-free medium (SF-900, available from Gibco, Gaithersburg, Md.) in a final volume of about 500 milliliters (ml). For Sf9:pVL1392-nhFcεRIα₁₋₅₂₈ cells grown in shaker flasks, TNM-FH medium was supplemented with pluronic F-68 (available from Pharmingen). For each virus stock used in protein production, the optimal amount of virus and harvest time post-infection was determined by small scale tests in 50 ml shaker flasks.

Recombinant protein PhFcεRIα₁₋₁₇₆ was produced as follows. Trichoplusia ni (Hi-5) cells were infected with recombinant virus BV:pVL1392-nhFcεRIα₁₋₅₂₈ that had been produced as described above to produce recombinant cell Hi-5:pVL1392-nhFcεRIα₁₋₅₂₈. Recombinant cell Hi-5:pVL1392-nhFcεRIα₁₋₅₂₈ was grown in shaker or spinner flasks for production of PhFcεRIα₁₋₁₇₆. Typical yields of PhFcεRIα₁₋₁₇₆ were about 2 to 12 milligrams per liter (mg/liter) of infected cells 2 to 4 days after infection.

Recombinant protein PhFcεRIα₁₋₁₇₆ was purified as follows. Supernatants from 1.5 to 5 liters of recombinant Hi-5:pVL1392-nhFcεRIα₁₋₅₂₈ cells were collected, filtered through 0.2 micron filters, and loaded directly onto a Mab15-1 (Sechi et al., 1996, J. Biol. Chem. 271, 19256-19263) monoclonal antibody column. Supernatants were recirculated over the column at least twice, followed by buffer (100 millimolar (mM) Na, K phosphate, pH 7) washes of about 300 ml, until the absorbance at 280 nanometers (nm) of the eluant returned to zero. PhFcεRIα₁₋₁₇₆ was eluted by two urea washes: 100 ml of 5 molar (M) urea in 100 mM phosphate, pH 7.0; then 100 ml of 7 M urea in 100 mM phosphate, pH 7.0; followed by extensive regeneration with 100 mM Na, K phosphate, pH 7.0. The urea eluants were pooled, concentrated to about 25 to 40 ml with an Amicon stirring concentrator, and dialyzed 4 times against 2 liters of 50 mM Tris, pH 7.5. The purity of PhFcεRIα₁₋₁₇₆ was verified by SDS-PAGE. Purified PhFcεRIα₁₋₁₇₆ was stored at 4° C. in the presence of 0.05% sodium azide. Final yield of PhFcεRIα₁₋₁₇₆ was about 50% based on an absorption coefficient of 2.6 mg⁻¹ml for the purified protein and the initial total protein estimated using ELISA assays with the initial cell supernatants.

An inhibition-ELISA assay was used to quantitate PhFcεRIα₁₋₁₇₆ expression and yields in initial transfected supernatants, subsequent viral amplifications and large scale protein preparations. In this assay, the binding of Mab15-1 antibody to the plated PhFcεRIα₁₋₁₇₆ protein was monitored using a goat anti-mouse-alkaline phosphatase antibody (A-2429, available from Sigma, St. Louis, Mo.). Unknown samples were used to compete for antibody binding and compared with a standard curve generated in parallel. Fifty microliters (ml or mL) of purified PhFcεRIα₁₋₁₇₆ was incubated in microtiter plates overnight at 4° C. at a concentration of 1 mg/ml in phosphate-buffered saline. Plates were rinsed with wash buffer containing 20 mM Hepes, pH 7.5, 100 mM NaCl, 0.1% Tween-20 (Hepes/NaCl buffer) and blocked with Hepes/NaCl buffer containing 1% Carnation dry milk. Standard inhibitor samples ranging from 0.1-50 mg/ml of PhFcεRIα₁₋₁₇₆ in two-fold dilution series were incubated with Mab15-1 (0.1 mg/ml final concentration) and added in duplicate to wells coated with PhFcεRIα₁₋₁₇₆. Standard controls included wells without overnight incubation with PhFcεRIα₁₋₁₇₆, and addition of Mab15-1 without inhibiting PhFcεRIα₁₋₁₇₆. Secondary antibody in a 1:5000 dilution was incubated after washing for 12 hour at room temperature. Plates were washed and developed using the AP reagent p-nitrophenyl phosphate (PNPP, available from Sigma 104-105). Microplates were read using a Molecular Devices SpectraMax Plus reader at 405 nm.

### Example 2

This Example describes the production of a FcεRIα protein crystal of the present invention.

Purified PhFcεRIα₁₋₁₇₆, produced as described in Example 1, was concentrated to a final concentration of 20 mg/ml in 20 mM Tris pH 7.5. Crystallization was carried out using the hanging drop method, with a precipitant composed of 100 mM Tris, pH 8.5, 200 mM NaOAc, and 18-24% PEG 4000. Crystals were obtained in 2 to 10 days amidst significant amounts of protein precipitate. At lower PEG concentrations, a different crystal form was observed. The crystals used in the structure determination typically grow as clusters of 3 to 20 crystals that could be separated manually. The crystals belong to the monoclinic space group C2, with cell dimensions of 88.6×69.6×49.3 angstroms, alpha=gamma=90 degrees, beta=116.7 degrees, with one receptor molecule per asymmetric unit. Such crystals diffracted to a resolution of about 2.4 angstroms. Crystals were harvested into harvest buffer containing 35% PEG 4000, 100 mM Tris pH 8.5. It is to be noted that the inventors produced and tested several hundred crystals using the various other proteins described in Example 1, before successfully obtaining the crystal described immediately above.

### Example 3

This Example describes the production of additional FcεRIα protein crystals of the present invention.

Nucleic acid molecule nhFcεRIα₁₋₅₁₆ , encoding the first 172 amino acids of the human FcεRIα protein was expressed in T. ni Hi-5 cells to produce PhFcεRIα₁₋₁₇₂ in a manner similar to that described for the production of PhFcεRIα₁₋₁₇₆ in Example 1. Purified PhFcεRIα₁₋₁₇₂ was concentrated to a final concentration of 20 mg/ml in 20 mM Tris pH 7.5. Crystallization was carried out using the hanging drop method, with a precipitant composed of 0.1-0.2 M NaAcetate, 0.1M Na Citrate, pH 5.6, 18-24% PEG, and HECAMEG detergent at it's Critical Micelle concentration (19.5 mM). Crystals were obtained in 2 to 10 days amidst significant amounts of protein precipitate. The crystals belong to the monoclinic space group P6122 with unit cell dimensions of 58×58×226 angstroms, alpha=beta=90 degrees; gamma=120 degrees, with one receptor molecule per asymmetric unit. Such crystals diffracted to a resolution of about 3.2 angstroms.

Using a different protocol, purified PhFcεRIα₁₋₁₇₆, produced as described in Example 1, was concentrated to a final concentration of 10 mg/ml in 20 mM Tris pH 7.5. Crystallization was carried out using the hanging drop method, with a precipitant composed of 100 mM Tris, pH 7.5, 0-20% isopropanol, and 18-24% PEG 4000. Crystals were obtained in 2 to 10 days amidst significant amounts of protein precipitate. The crystals belong to the monoclinic space group C2, with cell dimensions of 136.02×75.01×79.28 angstroms, alpha=gamma=90 degrees; beta=117.8 degrees. Such crystals diffracted to a resolution of about 3.0 angstroms.

### Example 4

This Example describes the production of a three-dimensional model of the present invention.

For data collection, crystals, produced as described in Example 2, were mounted in nylon loops (available from Hampton-Research, Laguna Niguel, Calif.) and rapidly cooled in liquid nitrogen after a short (about 30 second) soak in harvest buffer supplemented with 14% glycerol. Heavy atom soaks with K₂PtBr₄ and K₃AuCl₃ were done in harvest buffer with 5 mM Pt for 48 hours and 1 mM Au for 18 days. Data were collected at the Stanford Synchrotron Radiation Laboratories (SSRL) 7-1 beamline and at the Dupont-Northwestern-Dow undulator beamline at the Advanced Photon Source at Argonne National Laboratories. The statistics for these data are shown in Table 3.

TABLE 3
Crystallographic data and model refinement

| | Native | Au | Pt |
|---|---|---|---|
| Resolution | 2.4Å | 3.0Å | 4.0Å |
| Wavelength/Energy | 1.08/11.48 | 1.02/12.12 | 1.05/11.76 |
| (Å/ke V) | | | |
| Completeness, % | 96.9 (92.5) | 99.9 (100.0) | 96.3 (69.9) |
| (Last Shell) | | | |
| Ave. Redundancy | 3.9 (3.4) | 7.6 (7.3) | 6.2 (2.7) |
| (Last Shell) | | | |
| Rmerge, % | 5.7 (22.6) | 10.1 (39.8) | 5.1 (7.0) |
| (last shell) | | | |
| <I/sI> | 23.8 (4.5) | 19.0 (3.9) | 35.2 (15.9) |
| (last shell) | | | |
| DF/F (Resolution) | — | 0.218 (20-3Å) | 0.093 (20-4Å) |
| No. of sites | — | 1 | 1 |
| Phasing Power | — | 1.50/1.93 | 0.41/0.61 |
| acentric/centric | | | |
| Rcullis | — | 0.66/0.70 | 0.94/0.97 |
| acentric/centric | | | |

Overall Figure of Merit = 0.487
FOM after DM = 0.673
Refinement Statistics: 500-2.4Å
# Reflections (free) = 10247 (880)
Rfactor/Rfree, % = 24.2/27.1
# atoms = 1620
# waters = 126
RMSD bonds = 0.0077Å
RMSD angles = 1.530
Ave. B = 65.7Å²
R_{merge} = S|Iᵢ − <I>|/S|I|, where Iᵢ is the intensity of and individual reflection and <I> is the average intensity of that reflection.
R_{cryst} = S|Fp| − |F_{c}|/S|Fₚ|, where F_{c} is the calculated and Fₚ is the observed structure factor amplitude.
Phasing Power = F_{calc}/E, where F_{calc} = the heavy atom structure factor amplitude and E = the residual lack of closure error.
R_{cullis} = S||Fₚₕ − Fₚ| ±0 |F_{calc}|/S|Fₚₕ ± Fₚ|, where Fₚₕ is the derivative structure factor amplitude.

For the Pt and Au datasets, the wavelength was chosen to be 200 eV above the absorption edge of the metal, in order to maximize the anomalous signal for each heavy atom. Heavy atom data were collected using reverse beam protocols to optimize the anomalous diffraction signal. Diffraction data were collected with a Mar300 Image plate (SSRL) or a MarCCD detector (DND-CAT), and integrated and scaled with DENZO/SCALEPACK; see, Otwinowski et al., 1997, In Methods in Enzymology: Macromolecular Crystallography, part A, Academic Press, pp 307-326. The CCP4 suite of programs (Collaborative Computational Project, 1994, Acta Cryst. D50, 760-763) was used for further processing and identification of heavy atom sites. Otwinowski et al., ibid., and Collaborative Computational Project, ibid., are incorporated by reference herein in their entireties.

Heavy atom positions were identified from peaks in the anomalous and isomorphous difference patterson maps. Heavy atom positions were refined and phases calculated with the program MLPHARE, followed by solvent flattening and density modification with the program DM (Collaborative Computational Project, 1994, ibid.). The subsequent model was using the CNS program (Brunger et al., 1998, Acta Crystallogr D Biol Crystallogr 54, 905-921) with the combined maximum likelihood and experimental phase target (MLHL). Brunger et al., ibid., is incorporated by reference herein in its entirety. Specifically, the structure of the FcεRIα protein PhFcεRIα₁₋₁₇₆ was determined by multiple isomorphous replacement using gold and platinum heavy atom derivatives with the anomalous signal from both derivatives. The data collection and heavy atom phasing statistics are shown in Table 3. The MIRAS phases were used as input to the density modification program DM and the electron density map was of sufficient quality that the entire model except for two flexible loops and five residues at the termini could be built; see FIG. 1A and 1B. The model was further improved by cycles of automated refinement using the program CNS followed by manual rebuilding. The current R-factor and Rfree are 24.2% and 27.1% respectively for all the data to 2.4 angstroms. No electron density was observed for three residues at the N-terminus (1-3) and 2 residues at the C-terminus (175-176), and poor density was observed for two loops (residues 32-35 and 70-73) that are disordered in the crystal. Final statistics for the model are shown in Table 3.

### Example 5

This Example describes the structure of a FcεRIα protein predicted by a three-dimensional model of the present invention.

A. Overall Structure

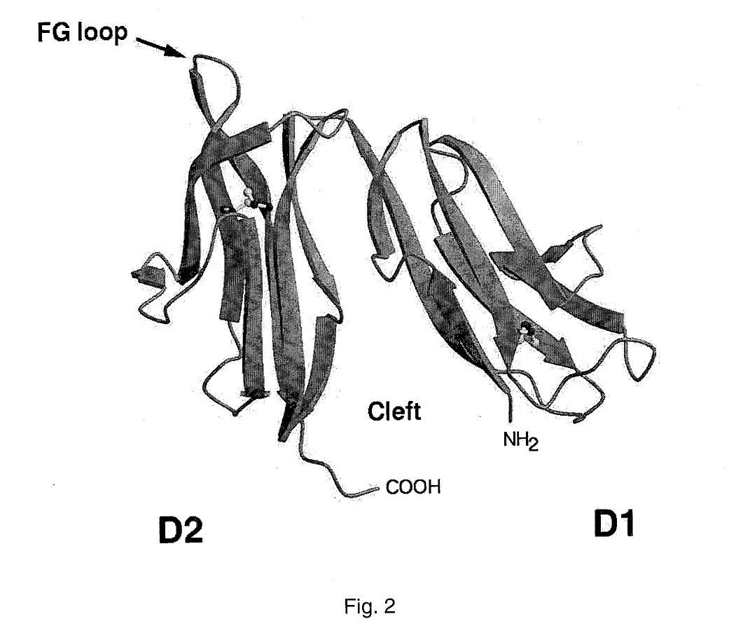

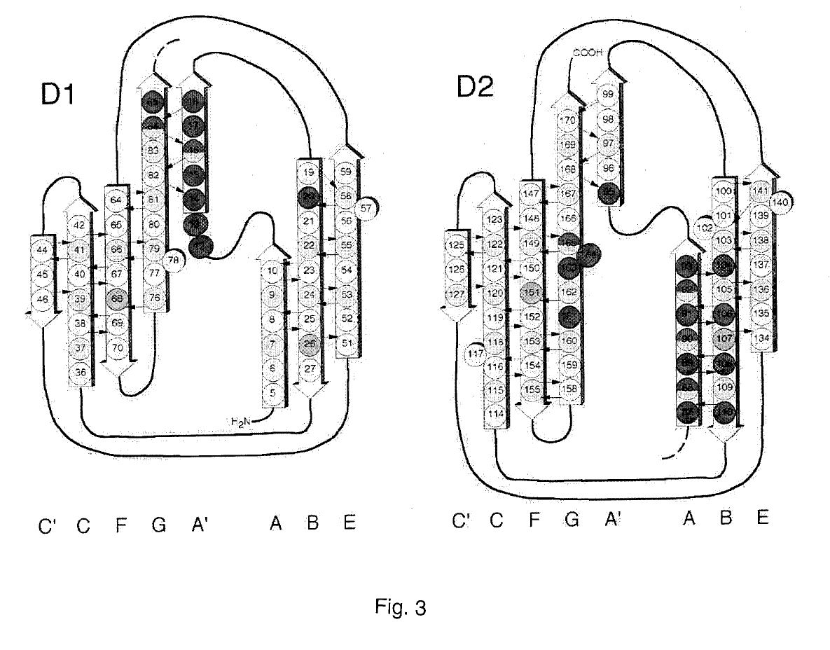

The model of extracellular domain of the human FcεRIα protein, also referred to herein as the hFcεRIα model or hFcεRIα structure, indicates that PhFcεRIα₁₋₁₇₆ is composed of two immunoglobulin (Ig) domains, D1 and D2, each about 85 residues in length, that are bent at an acute angle relative to each other and form an extended convex surface; see FIG. 2. The domain arrangement generates a flat surface at the top of the receptor that has been implicated in binding to the Fc domain of an IgE antibody. The domains are small compared to canonical variable and constant Ig domains and the shorter sequence is accommodated by truncation of the CC′E crossover region; see FIG. 2. Both domains D1 and D2 of the hFcεRIα model are composed of beta-strands AA′BCC′EFG, differing from the previously described I-set domains (Harpaz et al., 1994, J. Mol. Biol. 238, 528-539) by the absence of strand D. The nearly antiparallel domain packing places the A′B, CC′ and EF loops of D1 and the BC, C′E and FG loops of D2 near the top of the receptor; see FIG. 2. One feature of the topology is a crossover of the A strand from the ABE sheet to the CC′FG sheet, forming a short segment of parallel beta sheet in an otherwise antiparallel structure; see FIG. 2 and FIG. 3. In D1, the AA′ crossover make contacts in the D1D2 interface (see below), while in the D2 domain, residues in the A strand interact with D1; see FIG. 3.

Significant structural differences are also observed between D1 and D2 of the hFcεRIα model. The D1 and D2 sequences contain about 28% identical residues and superimpose with an RMS deviation of 1.2 angstroms for the Ca atoms. The F-G strands and loop differ between the two domains. In D2, these strands are longer and the FG loop projects above the D2 domain surface. The C′ strands also differ between the two domains. In D2, a series of aromatic residues (tyrosines at positions 120 and 131) form a hydrophobic core that pushes the C′ strand and loop away from the C strand, altering the local conformation of this region. The FG loop and C-C′ strands of D2 form part of the binding site for IgE (see below).

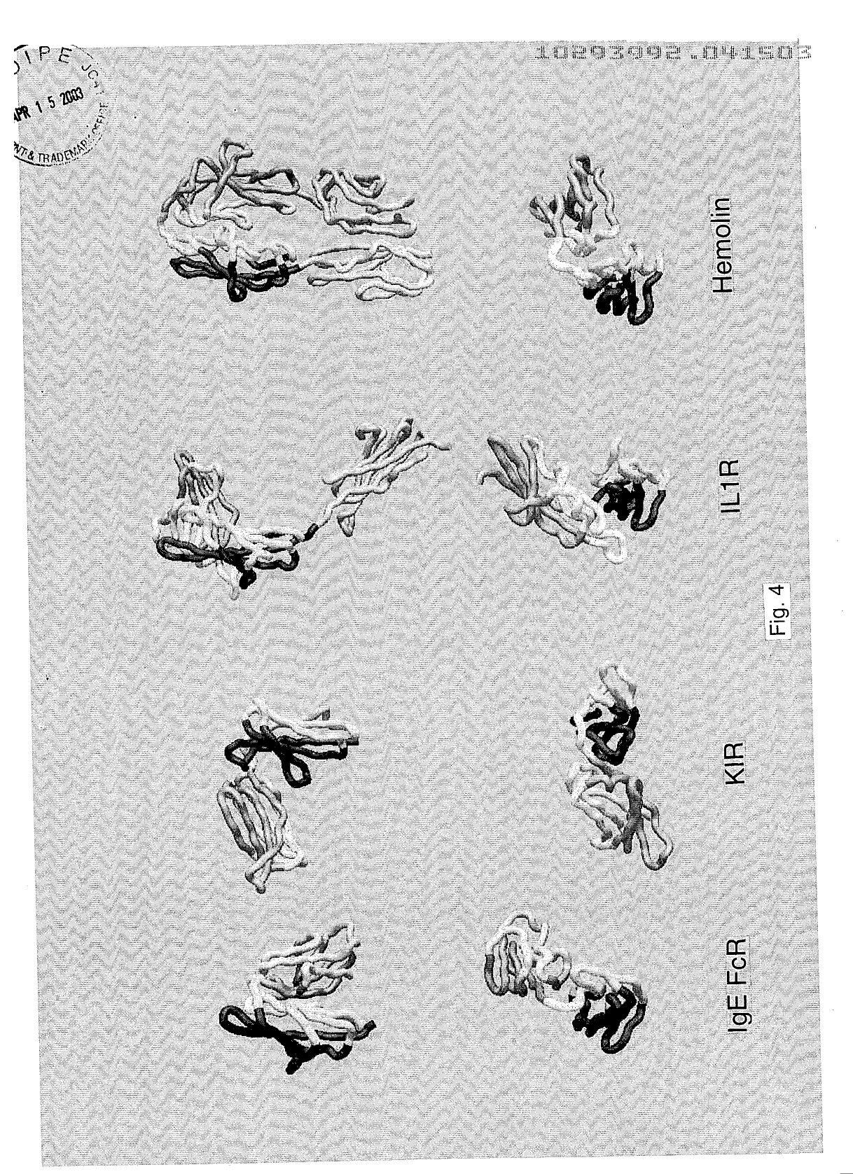

The tertiary packing arrangement of the hFcεRIα D1 and D2 domains is distinct from other tandem Ig domain structures; see FIG. 4. Comparison of the hFcεRIα model with other bent two-Ig domain structures reveals a high degree of variability in the bend angles and packing surfaces between domains. A subset of D1 and D2 representative structures of are shown in FIG. 4, including those from human FcεRIα (designated as IgE-FcR), the natural killer cell inhibitory receptor, (KIR, Fan et al., 1997, Nature 389, 96-100), the human growth hormone receptor (HGHBP, de Vos et al., 1992, Science 255, 306-312), the interleukin-1 receptor, (IL1R, Vigers et al., 1997, Nature 386, 190-194) and the insect defense protein hemolin (Su et al., 1998, Science 281, 991-995). The structures are oriented similarly with respect to the carboxyl-termini of the two Ig domains being compared. The figures on top show side views and the figures below show top views. The FcεRIα and hemolin structures have the most acute angles relating two sequential Ig domains. The top view of these domains shows that the orientation of the hemolin and FcεRIα domains are more closely related, but between this selected subset of proteins there is significant variability in the relative orientations of tandem Ig domain structures. The bend angle between domains and domain packing interfaces are clearly unique, and this variation is likely to be important in ligand binding interactions. For example, the FG loop of D2 in hFcεRIα is oriented quite differently with respect to D1 residues as compared to the same region of the KIR or HGHBP, thus changing the spatial relationships of D1D2 loops that may be involved in ligand interactions. To the inventors' knowledge, the hFcεRIα structure defines a new group of two sequential Ig domain structures that differs from other known tertiary arrangements.

B. The D1D2 Interface

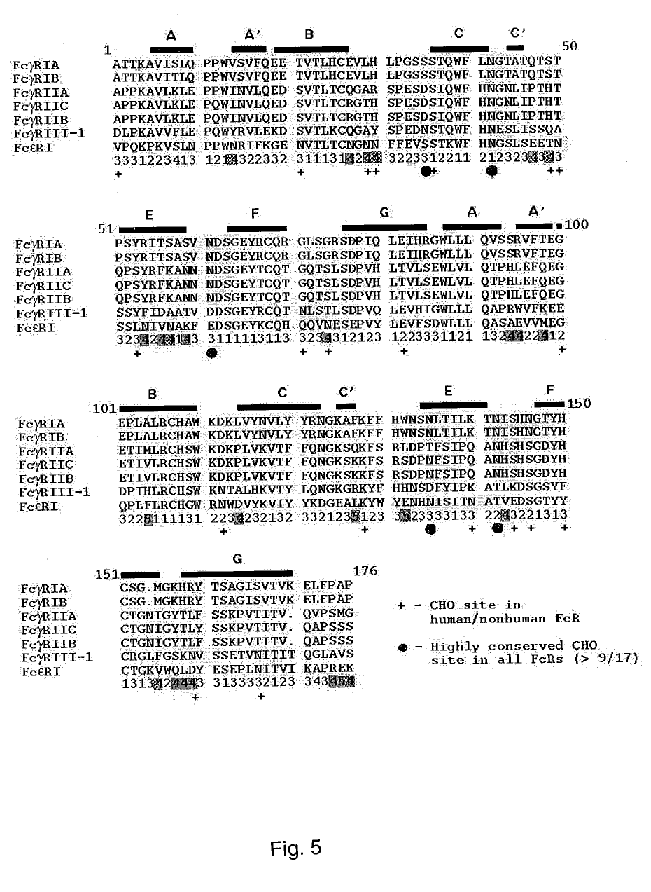

The bent shape of the FcεRIα structure produces a large interface between the D1D2 domains that buries 1280 Å² of accessible surface area of 28 D1 D2 residues. There are 11 residues from the D1 domain (12-18, 20, 84-86) and 17 residues from the D2 domain that are buried at the interface (87-93, 95, 104, 106, 108, 110-111, 161, 163-165). Of these 28 residues, 8 are completely conserved in all human FcgR and FcεRIα sequences (corresponding to residues 13, 87, 88, 90, 91, 106, 108, 110 of SEQ ID NO: 2); see FIG. 5. These conserved residues form a significant fraction to one side of the buried interface, suggesting that related FcRs would have a similar acute packing of the D1D2 domains as observed in the FcεRIα model.

However, 20 residues that form the D1D2 interface in the FcεRIα model differ in other FcRs and these differences could alter the relative orientations of the two domains. For example, the conserved tryptophan at position 110 packs against a phenylalanine at position 17 of FcεRIα. In related FcRs, this phenylalanine is changed to a leucine, which may lead to slight alterations in the packing of the two domains. Another central residue in the FcεRIα D1D2 interaction is residue R15, which forms a hydrogen bond with the carbonyl of L90 and is packed against L89, F84, and L165. In related human FcRs, arginine 15 is changed to serine or asparagine, which corresponds to a significant volume and charge change at the center of the D1D2 interaction. Since the interactions of the FcR with antibody are near the D1D2 interface, alterations in residues at the interface might influence receptor specificity. Other residues that are variable amongst the FcR sequences in the region of the D1D2 could also perturb the D1D2 interactions.

The bent hFcεRIα structure generates a cleft between the two domains that is near the trans-membrane anchor at the C-terminus of D2; see FIG. 2. This cleft is located far from the IgE binding site identified by mutagenesis studies (see below). Although there is no known function attributed to this region, while not being bound by theory, it is believed that this region could be a site of interaction with the extracellular regions of the beta or gamma subunits of the receptor. It has been suggested that interactions between the FcgRI and FcgRIIIA alpha and gamma subunits increase the binding affinity of the receptor for IgG (Miller et al., 1996, J. Exp. Med. 183, 2227-2233). Although the extracellular regions of the human FceR gamma chain are short (about 5 to 7 amino acids), these regions could potentially interact with the D1D2 cleft and thereby affect the affinity of the receptor for antibody. In addition, recent binding studies with recombinant, soluble FcεRIα and IgE have demonstrated a 10-fold lower affinity than had previously been determined in cell-binding assays (Cook et al., 1997, ibid.).

C. Carbohydrate Attachment Sites

The human FcεRIα protein PhFcεRIα₁₋₁₇₆ is the most highly glycosylated protein structure solved by X-ray crystallography to date, having seven N-linked glycosylation sites in 176 amino-acid residues. The intact FcεRIα on mast cells is approximately 40% carbohydrate by weight (Kanellopoulos, et al., 1980, E. J. Biol. Chem. 255, 9060-9066); LaCroix, et al., 1993, ibid.), with a heterogeneous molecular weight on SDS-PAGE gels of about 50 kilodaltons (kD). Human FcεRIα expressed in insect cells has a molecular weight of about 34 kD as observed using SDS-PAGE, but, based on typical insect cell glycosylation structures (-GlcNAc₂-Man₃-GlcNAc), could be expected to have a molecular weight of about 27.5 kD, suggesting the protein is about 30% carbohydrate by weight. While the presence of carbohydrate at the seven N-glycosylation sites is not required for binding to IgE (Letourneur et al., 1995, ibid.; Robertson, 1993, ibid.; Scarselli et al., 1993, ibid.), mutation of these sites or treatment of FceRI-expressing cells with tunicamycin leads to the aggregation of the receptor during biosynthesis.

In the hFcεRIα structure, carbohydrate density is observed at three of the seven predicted glycosylation sites. For two of these sites, asparagines 42 and 166, three sugar moieties were built. The carbohydrate at position 42 extends up towards the top of the FcεRIα structure, covering residues F60, S63 and V83. The carbohydrate attached to position 166 projects away from the protein surface, potentially as a result of crystal contacts and the modification of the third and sixth positions of the first GlcNac residue. The third carbohydrate attachment site is the arginine at position 21.

Many of the related FcR proteins are also highly glycosylated proteins and the glycosylation sites vary between receptors. Rat and mouse FcεRIα proteins each have six potential N-linked glycosylation sites, of which two sites and one site, respectively are shared in common with the human FcεRIα protein. Comparison of seventeen human and animal FcR sequences identifies twenty-five different potential N-linked carbohydrate attachment sites in related FcRs. The twenty-five sites are distributed evenly between D1 and D2, with fourteen sites in D1 and eleven sites in D2. Five of these sites are relatively well conserved sites in all FcRs (found in >9/17 sequences analyzed) and they correspond to residues 35, 42, 61, 135, and 142 of SEQ ID NO: 2. These sites cover a significant fraction of the FcεRIα surface on both major faces of D1 and D2, placing limitations on the surface available for interactions with antibody.

It is not known why FcRs are so heavily glycosylated. Many potential roles for carbohydrate sites on proteins have been suggested, including specific roles in determining the tertiary (Wyss et al., 1995, Science 269, 1273-1278) or quaternary structures of proteins (Huber et al., 1976, Nature 264, 415-420; Vaughn et al., 1998, Structure 6, 63-73). In the case of the human FcRs, the number of potential N-linked glycosylation sites correlates to some degree with the affinity of the FcR for immunoglobulin. Table 4 shows the number of glycosylation sites in the domains corresponding to the extracellular domain of the human FcεRIα protein along with the total number of glycosylation sites in parentheses. Affinity data are taken from Ravetch et al., 1998, ibid.; Ravetch et al., 1991, Annu. Rev. Immunol. 9, 457-492.

TABLE 4
Comparison of the number of predicted glycosylation sites and
the affinity of different FcRs for antibody.

| | | |
|---|---|---|
| | # CHO sites | |
| FcR | (total) | Affinity |

Human

| | | |
|---|---|---|
| FcεRI | 7 | high (10⁻⁹-10⁻¹⁰ M) |
| FcγRIA (CD64) | 5 (7) | high (3 domains, 10⁻⁸-10⁻⁹ M) |
| FcγRIB (CD64) | 5 (7) | high (3 domains, 10⁻⁸-10⁻⁹ M) |
| FcγRIIA (CD32) | 2 (3) | low (10⁻⁶ M) |
| FcγRIIB (CD32) | 3 | low (10⁻⁶ M) |
| FcγRIIC (CD32) | 3 (4) | low (10⁻⁶ M) |
| FcγRIIIA (CD16) | 5 (6 in variant) | low (10⁻⁶ M) |

Mouse

| | | |
|---|---|---|
| FcεRI | 6 | high (10⁻⁹-10⁻¹⁰ M) |
| FcγRI | 4(5) | high (3 domains, 10⁻⁷-10⁻⁸ M) |
| FcγRIIb | 4(5) | low (10⁻⁶ M) |
| FcγRIIfa | 4 | low (10⁻⁶ M) |

Rat

| | | |
|---|---|---|
| FcεRI | 7 | high (10⁻⁹-10⁻¹⁰ M) |
| FcγRII | 6 (7 total) | low |
| FcγRIII | 5 | low |

Other

| | | |
|---|---|---|
| FcγRII (guinea pig) | 5 (6) | low |
| FcγRIII (pig) | 3 | low |
| FcγRII (bovine) | 6 | low |

In the high affinity FcRs, there are typically 5 to 7 potential N-linked glycosylation sites, whereas in the low affinity FcRs there are as few as two sites. One significant difference in the function of the high and low affinity FcRs is the probability that they will bind antibody in the absence of antigen. The high affinity receptors such as FcεRI can bind IgE prior to interacting with antigens. While not being bound by theory, it is believed that since FcR activation requires crosslinking of receptors, glycosylation might prevent the aggregation of large antibodies at the cell surface bound by FcRs. Crystallization of proteins at lipid/water interfaces can occur readily, and the potentially high local concentrations of membrane-bound antibodies might lead to FcR activation in the absence of antigen. The low affinity IgG receptors interact with antibody-antigen aggregates that can simultaneously bind and activate multiple FcRs. While not being bound by theory, it is believed that glycosylation may not be quite as important for these receptors, since interaction with the antibody could occur after the binding of antigen.

However, it is likely that there are additional explanations for the glycosylation that is observed in the FcRs. The non-human FcRs do not show an obvious correlation of the number of carbohydrate sites and FcR affinity. While not being bound by theory, it is believed that glycosylation might be important in FcR signaling, by orienting receptor:antibody complexes into functional signaling complexes (i.e. by preventing antigen-crosslinked complexes from forming non-functional aggregates). It is known that activation through FcεRI is sensitive to some geometrical constraints imposed by antigen crosslinkers, although the nature of these physical constraints is poorly understood. The recent crystal structure determination of an erythropoietin-receptor complex suggests that the orientation of ligand-mediated dimerization of cell-surface receptors may be important in efficient signal transduction (Syed et al., 1998, Nature 395, 511-516).

D. Receptor Binding Site for IgE (IgE Binding Domain)

A number of mutagenesis studies have been carried out in an effort to elucidate the regions of the FceRI that are critical for the interaction with IgE molecules (Cook et al., 1997, ibid.; Hulett et al., 1993, ibid.; Hulett et al., 1994, ibid.; Hulett et al., 1995; Mallamaci et al., 1993, ibid.). These experiments have demonstrated an important role for amino acids in the D2 domain of the receptor, although some regions of D1 are also likely to be involved in IgE binding. Studies suggesting that D1 plays a role in IgE binding include the deletion of D1 (Robertson, 1993, ibid.; Scarselli et al., 1993, ibid.) or substitution with a homologous IgG receptor (Hulett et al., 1994, ibid.). These experiments do not determine conclusively whether D1 interacts directly with the IgE or whether D1 indirectly alters the structure of D2 and subsequent interactions with IgE. Analysis of the FcεRIα model of the present invention is needed to predict important IgE binding regions in the protein. For example, the substitution or elimination of residues at the D1D2 interface can influence D2 interactions with antibody Fc regions.

In addition, there are a number of regions of D1 which have been excluded as determinants of the specificity of the receptor for IgE, since these FcεRIα segments can be substituted by the corresponding residues in the FcgRIIIA protein (Mallamaci et al., 1993, ibid.). These residues include segments 25-38, 43-54, 67-79, and 77-86. Substitution of residues 10-21 or 55-67 disrupt the binding of IgE and 5 different monoclonal antibodies, suggesting that residue differences in these segments may affect the folding of hybrid molecules. The 3-D models of the present invention, however, are needed to conduct an amino acid by amino acid analysis of which residues actually directly interact with IgE antibodies.

The FcεRIα residues which have been implicated in past studies include residues in the D2 BC loop (amino acid 115), in the C strand (amino acids 117, 118, 120-123), in the C′E loop (amino acids 129, 131), the F strand (amino acids 149, 153) and the FG loop (amino acids 155 and 159) (Cook et al., 1997, ibid.; Hulett et al., 1994, ibid.; Hulett et al., 1995, ibid.). In addition, residues 87 (at the D1D2 interface) and 128 (in the C′E loop) are likely to be part of the IgE interaction site, since mutation of these residues have been shown to influence receptor binding to the IgE point mutant R334A (Cook et al., 1997, ibid.). Furthermore, a synthetic peptide corresponding to residues 119-129 has been demonstrated to block IgE binding to the FcεRIα, with an apparent K_{D} of about 3 mM (McDonnell et al., 1997, ibid.; McDonnell et al., 1996, ibid.).

Analysis of the hFcεRIα model, however, is needed to indicate that of the fifteen residues (i.e., amino acids 87, 115 117, 118, 120-123, 128, 129, 131, 149, 153, 155 and 159), six are buried in the protein core (i.e., amino acids 115, 118, 120, 131, 149, 155) and predicts that substitution at these positions may lead to indirect structural changes that affect IgE binding. Three of the residues are either partially buried or glycine (i.e., amino acids 122, 129, 153), and substitution may affect the conformation of the local residues. The remaining residues (i.e., amino acids 87, 117, 121, 123, 128, 159) are all exposed amino acids at the FcεRIα surface. All of the implicated residues form a contiguous surface extending from the back side of the D2 domain to the top region near the D1D2 interface. Four of the residues are conserved in all human FcRs (i.e., amino acids 87, 118, 149, and 153) and may define a set of common interactions between all FcR receptors and their target Ig molecules.

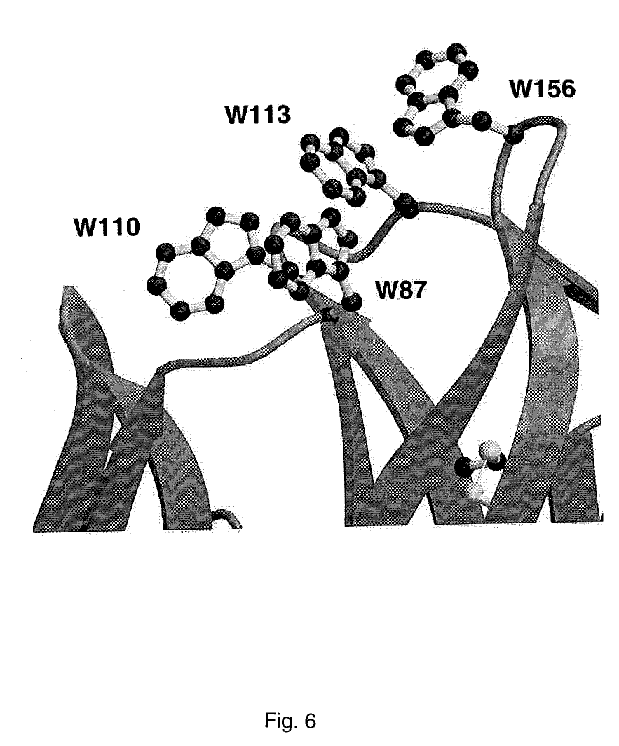

The hFcεRIα model also indicates that the region of the D2 domain defined by mutagenesis also borders on a number of surface accessible aromatic residues, including four prominent tryptophans at the top of the FcεRIα molecule, namely residues 87, 110, 113, and 156. These four tryptophans form a flat, hydrophobic ridge that neighbors the D2 FG loop. This unusual arrangement of four surface tryptophans probably forms a contact surface for a complementary interaction with an IgE antibody. Tryptophan 87 has already been implicated by mutagenesis studies and tryptophan 156 is prominently displayed at the top of the FG loop. Tryptophan 156 is a glycine in all FcgRs and grafting of residues 154-161 of the FcεRIα FG-loop to FcgRII confers IgE binding. It is to be noted, however, that such a graft does not eliminate IgG binding. The hFcεRIα model predicts that other amino acids, e.g., the tryptophan at residue 87, may be important for antibody class recognition specificity. Other exposed aromatic residues are found concentrated near the IgE binding domain; FIG. 6 shows a surface representation of all of the exposed aromatic groups in the hFcεRIα structure, clearly outlining the tryptophan ridge and residues in D2 near the CC′E region.

E. Implications for the Binding of Other FcRs

Since carbohydrate would be expected to disrupt any close-packed protein:protein interface, it is interesting to compare the known carbohydrate sites with the proposed IgE-binding site on the receptor surface as defined by models of the present invention. The positions of the carbohydrate attachment sites for seventeen related FcRs indicated that the N-linked carbohydrate sites delineate a boundary around the proposed IgE binding site. This is consistent with the suggestion that related FcRs share a common binding surface for antibody molecules. Studies of the FcgRII specificity for IgG, for example, have implicated the following residues: amino acids 113-116, 129, 131, 133, 134, 155, 156, and 159-161 (Hulett et al., 1994, ibid.; Hulett et al., 1995, ibid.). In addition, domain-swap experiments have demonstrated that two of the related FcgRs can form functional hybrid molecules with FcεRIα (Hulett et al., 1995, ibid.; Mallamaci et al., 1993, ibid.), suggesting that these receptors share a common binding surface with their respective antibody ligands. Once again, however, it should be noted that only with the model can one predict exactly which FcR residues directly interact with an Fc domain of an antibody.

The hFcεRIα model indicates that the top of the FcR structure is devoid of carbohydrate-attachment sites in the region of D2 that has been implicated in direct interactions with Ig molecules. The neighboring surface of the D1 domain including loops A′B and EF, are also devoid of carbohydrate and could form part of an extended antibody binding site across the D1D2 interface. If these D1 loops are important in determining the specificity and affinity of the FcR:antibody interaction, one might observe sequence variability between high affinity and low IgG receptors and the IgE receptor. This variability in the human IgG and IgE receptors is shown in FIG. 5. For residues 3-173 of the hFcεRIα protein, there are 73 amino acid differences that are unique to the IgE receptor as compared to any of the IgG receptors and these are indicated below the sequence alignments. Of these 73 amino acids unique to the human FcεRIα protein, 27 positions are highly variable in the different FcR sequences (>4/7 different amino acids. There are five regions that stand out with clusters of variable residues:residues 27-30, 47-49, 54-59, 94-98 and 155-159. Residues 155-159 (the FG loop) are highly variable and do at least partially determine the specificity of FcR interactions. It is again to be noted that without the model one cannot determine which regions of sequence variability are important in determining FcR protein functional domains.

Previous experiments have shown that residues 27-30 and 47-49 are not critical for FcR specificity (Mallamaci et al., 1993, ibid.), and the presence of glycosylation sites within these segments in related FcRs support the suggestion that these regions are not part of the FcR:antibody interaction. The hFcεRIα model indicates that residues 94-98 are found in the A′ strand near the D1D2 cleft and therefore are not likely to interact with antibody directly, but these residues might influence interactions indirectly by altering the D1D2 packing interface.

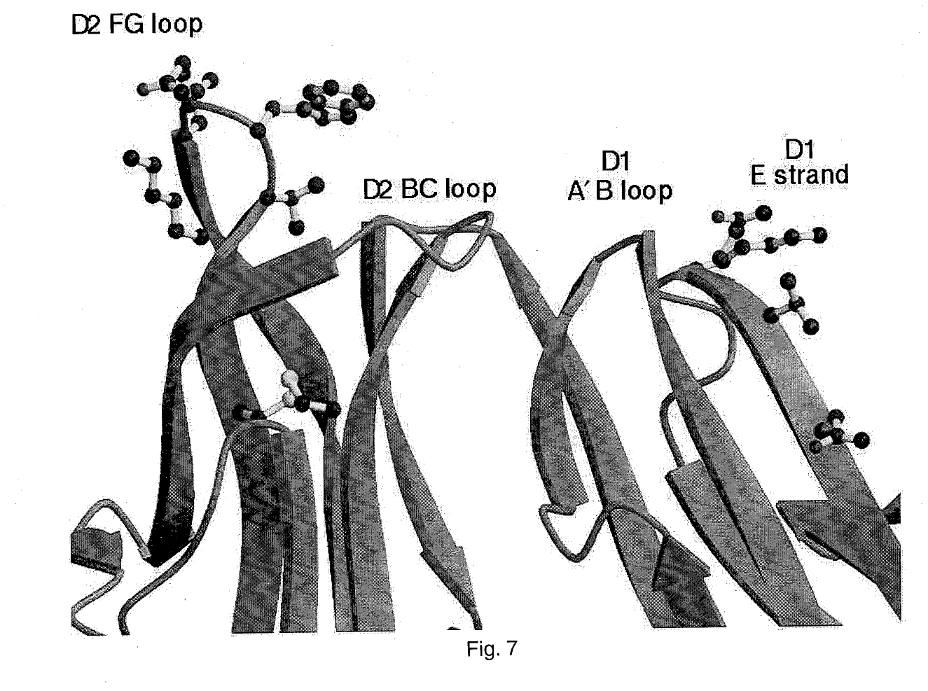

The remaining group of highly variable residues (54-59) are in the D1 E strand (see FIG. 7), near the FcεRIα binding site as predicted by the hFcεRIα model. Residues 54-59 could form a D1 surface of interaction with the Fc domains of antibodies, extending the binding site across both FcεRIα domains. This prediction is supported by a study reporting that the exchange of FcεRIα residues 55-67 with residues from the FcgRIIIA receptor disrupts the folding of the protein (Mallamaci et al., 1993, ibid.), as some of the residue changes form part of the D1 hydrophobic core. The hFcεRIα model also predicts that the neighboring D1 A′B loop (residues 18-21) could also form part of an extended surface of interaction with the antibody. Thus, models of the present invention are needed to interpret data from mutagenesis and swapping experiments.

F. Stoichiometry of Binding Between FcR and Antibody

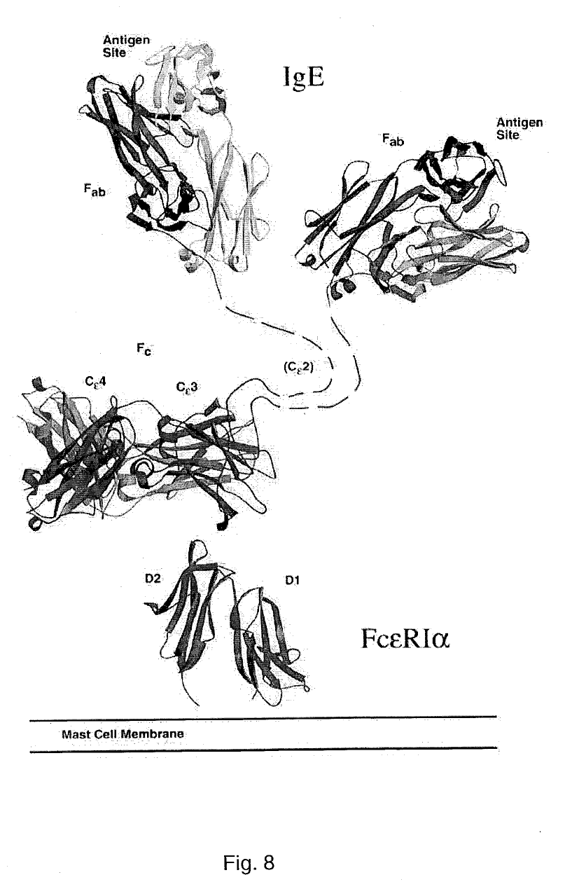

The activation of FcR-bearing cells requires crosslinking of the receptors, which leads to the activation of intracellular kinase cascades analogous to those in B and T cells. For both high and low high affinity receptors FceRI and FcgRIII, a stoichiometry of 1:1 is observed between the receptor and the Fc domains of the respective antibodies to which they bind (Ghirlando et al., 1995, Biochemistry 34, 13320-13327; Kanellopoulos et al., 1980, ibid.; Keown et al., 1997, Eur. Biophys. J. 25, 471-476), consistent with a requirement for antigen to cause receptor aggregation and activation. The binding site on the Fc domain of an IgE antibody for its receptor has been extensively studied by mutagenesis, implicating amino acids in the third constant domain (Ce3) of the IgE (Basu et al., 1993, J. Biol. Chem. 268, 13118-13127; Henry et al., 1997, Biochemistry36, 15568-15578; Nissim et al., 1991, Embo J. 10, 101-107; Presta et al., 1994, J. Biol. Chem. 269, 26368-26373). The structure of the Fc domain of IgE antibodies (also referred to as IgE-Fc domains) has not been experimentally determined, but is homologous to the Fc domain of IgG antibodies (also referred to as IgG-Fc domains), for which a number of crystal structures are available (Harris et al., 1998, J. Mol. Biol. 275, 861-872; Huber et al., 1976, Nature 264, 415-420). The residues of the IgE-Fc domain implicated in binding to FceRs based on mutagenesis analysis are shown mapped onto the structure of the IgG-Fc domain in FIG. 8. The site on an IgG-Fc domain to which FcgRI and FcgRII receptors bind has been mapped to a similar, although smaller, surface that primarily includes residues in the hinge region before the Cg2 domain (Canfield et al., 1991, J. Exp.Med. 173, 1483-1491; Duncan et al., 1988, Nature 332, 563-564; Jefferis et al., 1990, Mol. Immunol. 27, 1237-1240; Lund et al., 1991, J. Immunol. 147, 2657-2662).

An antibody Fc domain is a homodimeric structure that is significantly larger than its respective FcR; see FIG. 8. The observed 1:1 stoichiometry between receptor and antibody indicates that the two-fold symmetry of the Fc domain does not lead to the binding of two FcRs, even with isolated molecules in solution. Without being bound by theory, it is believed that the large and convex nature of the FcR binding surface could span two antibody domains (Cg2 in IgG and Ce3 in IgE) and induce a conformational change in the Fc domain that would prevent the binding of a second FcR to the same antibody. The FcR structure could also form an asymmetric complex with the antibody that sterically blocks the binding of a second FcR, perhaps using the protruding FG loop to block symmetric interactions with the Fc hinge region.

### Example 6

This Example describes the production of additional three-dimensional models of the present invention as well as descriptions of FcεRIα proteins predicted therefrom.

A. Production and description of a crystal of PhFcεRIα₁₋₁₇₂ that belongs to tetragonal space group P4₃, with a=b=145.08 Å, c=62.74 Å, a=b=g=90°, referred to herein as crystal form T1.

Protein PhFcεRIα₁₋₁₇₂, having SEQ ID NO: 4, was produced using techniques known to those skilled in the art by a lec1 hamster ovary (CHO) cell line transformed with a nucleic acid molecule encoding the protein, i.e., a nucleic acid molecule comprising SEQ ID NO: 3. Crystals were grown in a manner similar to that described in Example 2 via vapor diffusion using a well solution of 20% to 32% PEG 10000, 0.1 M ammonium citrate pH 5.6, and 0.1 M sodium chloride, and a protein starting concentration of 5 to 10 mg/ml. Other size PEGs from 4000 to 20000 were also used, as well as sodium citrate pH 5.6 as a buffer. Other salts such as sodium acetate and ammonium sulfate were also used to grow crystals. The crystal used in the structure determination, analyzed in a manner similar to that described in Example 4, had five copies of the receptor in the crystallographic asymmetric unit and diffracted to a maximum resolution of 3.1 Å. This crystal form, form T1, was refined to a crystallographic R_{free}/R_{work} of 32.78%/29.19% using all the observed data |F|>0 to 3.1 Å and a non-crystallographic symmetry (NCS) restraint constant of 300 kcal/mol Å² for all atoms. There were no waters included in the model. The atomic coordinates of PhFcεRIα₁₋₁₇₂, form T1, are listed in Table 5. The solvent accessibilities of the amino acids of PhFcεRIα₁₋₁₇₂, form T1, are indicated in Table 9. Table 13 provides crystallographic data and model refinement statistics of PhFcεRIα₁₋₁₇₂, form T1. A root mean square (rms) deviation analysis of the alpha carbon positions of PhFcεRIα₁₋₁₇₂, form T1, as compared to PhFcεRIα₁₋₁₇₆, form M1, is shown in Table 14. The first line is an overall rms on the segments that align in space. The second two lines are the rms deviations for the loops when the molecules are superimposed according to the first line. Only one copy of model in T1 is compared because the models do not differ by much because of tight NCS restraints.

B. Production and description of a crystal of PhFcεRIα₁₋₁₇₂ that belongs to tetragonal space group P4₃, with a=b=150.50 Å, c=74.18 Å, α=β=γ=90°, referred to herein as crystal form T2.

Protein PhFcεRIα₁₋₁₇₂, having SEQ ID NO: 4, was produced using techniques known to those skilled in the art by a lec1 Chinese hamster ovary (CHO) cell line transformed with a nucleic acid molecule encoding the protein, i.e., a nucleic acid molecule comprising SEQ ID NO: 3. Crystals were grown and analyzed as described in Example 6A. The crystal used in the structure determination had five copies of the receptor in the crystallographic asymmetric unit and diffracted to a maximum resolution of 3.8 Å. This crystal form, form T2, was refined to a crystallographic R_{free}/R_{work} of 30.64%/27.99% using all the observed data |F|>0 to 3.8 Å and a NCS restraint constant of 300 kcal/mol Å² for all atoms. There were no waters included in the model. The atomic coordinates of PhFcεRIα₁₋₁₇₂, form T2, are listed in Table 6. The solvent accessibilities of the amino acids of PhFcεRIα₁₋₁₇₂, form T2, are indicated in Table 10. Table 13 provides crystallographic data and model refinement statistics of PhFcεRIα₁₋₁₇₂, form T2. A rms deviation analysis of the alpha carbon positions of PhFcεRIα₁₋₁₇₂, form T2, as compared to PhFcεRIα₁₋₁₇₆, form M1, is shown in Table 14.

C. Production and description of a crystal of PhFcεRIα₁₋₁₇₆ that belongs to monoclinic space group C2, with a=136.90 Å, b=73.79 Å, c=79.40 Å, α=γ=90°, and β=117.74°, referred to herein as crystal form M2.

Protein PhFcεRIα₁₋₁₇₆, having SEQ ID NO: 2, was produced in T ni Hi-5 cells as described in Example 1. Crystals were grown in a manner similar to that described in Example 2 via vapor diffusion using a well solution of 12% to 20% PEG 4000, 0.1 M HEPES (or Tris) pH 7.5, and 0 to 10% isopropanol, and a protein starting concentration of 5 to 30 mg/ml. The crystal used in the structure determination, analyzed in a manner similar to that described in Example 4, had two copies of the receptor in the crystallographic asymmetric unit and diffracted to a maximum resolution of 3.2 Å. This crystal form, form M2, was refined to a crystallographic R_{free}/R_{work} of 28.30%/25.69% using all the observed data |F|>0 to 3.2 Å. A NCS restraint constant of 300 kcal/mol Å² has been imposed for all atoms except certain ones in loops and crystal contacts (residues 1-3, 27-38, 41-43, 69-75, 87, 98, 111-117, 125-135, 144, 152-158 of SEQ ID NO: 2) and the N-linked carbohydrate atoms. There were no waters included in the model. The atomic coordinates of PhFcεRIα₁₋₁₇₆, form M2, are listed in Table 7. The solvent accessibilities of the amino acids of PhFcεRIα₁₋₁₇₆, form M2, are indicated in Table 11. Table 13 provides crystallographic data and model refinement statistics of PhFcεRIα₁₋₁₇₆, form M2. A rms deviation analysis of the alpha carbon positions of PhFcεRIα₁₋₁₇₆, form M2, as compared to PhFcεRIα₁₋₁₇₆, form M1, is shown in Table 14.

D. Production and description of a crystal of PhFcεRIα₁₋₁₇₂ that belongs to hexagonal space group P6₁22, with a=b=5 8.62 Å, c=229.19 Å, α=β=γ=90°, referred to herein as crystal form H1.

Protein PhFcεRIα₁₋₁₇₂, having SEQ ID NO: 4 except that the isoleucine at position 170 was replaced with cysteine, was produced in a manner similar to that described in Example 1, except that Spodoptera frugiperda Sf9 cells were used instead of T. ni Hi-5 cells. Crystals were grown in a manner similar to that described in Example 2 via vapor diffusion using a well solution of 20% to 30% PEG 4000, 0.1 M sodium citrate pH 5.6, 0.1 M sodium chloride, and 5-40 mM Methyl-6-O-(N-heptylcarbamoyl)-a-D-glucopyranoside (HECAMEG), a protein starting concentration of 10 mg/ml. The crystal used in the structure determination, analyzed in a manner similar to that described in Example 4, had one copy of the receptor in the crystallographic asymmetric unit and diffracted to a maximum resolution of 3.2 Å. This crystal form, form H1, was refined to a crystallographic R_{free}/R_{work} of 31.27%/28.78% using all the observed data |F|>0 to 3.2 Å. The atomic coordinates of PhFcεRIα₁₋₁₇₂, form H1, are listed in Table 8. The solvent accessibilities of the amino acids of PhFcεRIα₁₋₁₇₂, form H1, are indicated in Table 12. Table 13 provides crystallographic data and model refinement statistics of PhFcεRIα₁₋₁₇₂, form H1. A rms deviation analysis of the alpha carbon positions of PhFcεRIα₁₋₁₇₂, form H1, as compared to PhFcεRIα₁₋₁₇₆, form M1, is shown in Table 14.

E. The principal differences in the structures from the various crystal forms occurred in the BC loop in domain 1 (the “30 loop”), the C′ strand in domain 2 (the “130 region”) and the carbohydrate sites. There were also smaller differences in the termini of the structures and the FG loop in domain 1 (the “72 loop”).

The 30 loop showed the greatest variability across the different space groups. The density for this loop was often the poorest density in the map, suggesting that the loop may vary in conformation even within a single crystal. In T1 and T2, the density for this loop was higher than the rest (when the molecule was viewed in the normal orientation, with the FG loop of domain 2 at the top and the cleft between the domains at the bottom.) In the tetragonal cells, the 30 loop passed close to residue 51. In the two copies of the receptor in the larger monoclinic cell M2, the 30 loop was pulled down by crystallographic contacts. In these two copies, the density for the 30 loop clearly showed the loop was pulled away from the rest of the molecule to reveal an empty space inside the loop. The location of the 30 loops in H1 and M1 was intermediate to those of the tetragonal cells and M2.

The 130 strand varied across the crystal forms as well. In T1, T2, and the B copy in M2, this strand hydrogen bonded with the C strand in domain 2 to form a canonical C′ strand. In the H1 form, the strand crossed over to the other side of the sheet to form a D strand. In the forms M1 and the A copy of M2, this strand was intermediate to a canonical C′ and D strand.

The density at the termini tended to be poorly ordered, but the M2 crystal showed density for the N-terminus. All of the other models began at amino acid 4. The M1 and M2 models were built to residue 174 out of 176 total residues, the H1 model was built to the C-terminal residue 172, and the two tetragonal forms have models that were built to residue 171 out of 172 total residues.

TABLE 5
Atomic coordinates of PhFcεRIα₁₋₁₇₂, Form T1

| ATOM | ATOM | | | | | | | |
|---|---|---|---|---|---|---|---|---|
| NUMBER | TYPE | RESIDUE | # | X | Y | Z | OCC | B |
| 1 | CB | LYS C | 4 | 14.321 | 45.864 | 45.068 | 1.00 | 151.11 |
| 2 | CG | LYS C | 4 | 15.396 | 44.881 | 44.650 | 1.00 | 151.11 |
| 3 | CD | LYS C | 4 | 16.203 | 44.418 | 45.852 | 1.00 | 151.11 |
| 4 | CE | LYS C | 4 | 17.285 | 43.425 | 45.453 | 1.00 | 151.11 |
| 5 | NZ | LYS C | 4 | 18.066 | 42.968 | 46.639 | 1.00 | 151.11 |
| 6 | C | LYS C | 4 | 12.828 | 45.080 | 43.246 | 1.00 | 214.46 |
| 7 | O | LYS C | 4 | 12.702 | 44.022 | 43.863 | 1.00 | 214.46 |
| 8 | N | LYS C | 4 | 12.367 | 47.226 | 44.431 | 1.00 | 214.46 |
| 9 | CA | LYS C | 4 | 13.426 | 46.310 | 43.920 | 1.00 | 214.46 |
| 10 | N | PRO C | 5 | 12.448 | 45.209 | 41.965 | 1.00 | 98.70 |
| 11 | CD | PRO C | 5 | 12.271 | 46.470 | 41.224 | 1.00 | 125.98 |
| 12 | CA | PRO C | 5 | 11.863 | 44.086 | 41.229 | 1.00 | 98.70 |
| 13 | CB | PRO C | 5 | 10.998 | 44.785 | 40.181 | 1.00 | 125.98 |
| 14 | CG | PRO C | 5 | 11.793 | 45.997 | 39.866 | 1.00 | 125.98 |
| 15 | C | PRO C | 5 | 12.912 | 43.157 | 40.611 | 1.00 | 98.70 |
| 16 | O | PRO C | 5 | 14.063 | 43.545 | 40.398 | 1.00 | 98.70 |
| 17 | N | LYS C | 6 | 12.509 | 41.923 | 40.330 | 1.00 | 208.77 |
| 18 | CA | LYS C | 6 | 13.417 | 40.948 | 39.747 | 1.00 | 208.77 |
| 19 | CB | LYS C | 6 | 14.011 | 40.068 | 40.851 | 1.00 | 249.20 |
| 20 | CG | LYS C | 6 | 15.074 | 39.104 | 40.363 | 1.00 | 249.20 |
| 21 | CD | LYS C | 6 | 15.769 | 38.385 | 41.512 | 1.00 | 249.20 |
| 22 | CE | LYS C | 6 | 16.860 | 37.456 | 40.986 | 1.00 | 249.20 |
| 23 | NZ | LYS C | 6 | 17.633 | 36.780 | 42.068 | 1.00 | 249.20 |
| 24 | C | LYS C | 6 | 12.709 | 40.087 | 38.703 | 1.00 | 208.77 |
| 25 | O | LYS C | 6 | 11.779 | 39.341 | 39.022 | 1.00 | 208.77 |
| 26 | N | VAL C | 7 | 13.159 | 40.207 | 37.454 | 1.00 | 73.65 |
| 27 | CA | VAL C | 7 | 12.599 | 39.454 | 36.315 | 1.00 | 73.65 |
| 28 | CB | VAL C | 7 | 13.163 | 39.923 | 34.968 | 1.00 | 90.39 |
| 29 | CG1 | VAL C | 7 | 12.395 | 39.255 | 33.860 | 1.00 | 90.39 |
| 30 | CG2 | VAL C | 7 | 13.095 | 41.425 | 34.847 | 1.00 | 90.39 |
| 31 | C | VAL C | 7 | 12.876 | 37.955 | 36.338 | 1.00 | 73.65 |
| 32 | O | VAL C | 7 | 14.017 | 37.539 | 36.224 | 1.00 | 73.65 |
| 33 | N | SER C | 8 | 11.833 | 37.148 | 36.461 | 1.00 | 91.19 |
| 34 | CA | SER C | 8 | 12.002 | 35.707 | 36.469 | 1.00 | 91.19 |
| 35 | CB | SER C | 8 | 11.113 | 35.074 | 37.541 | 1.00 | 89.05 |
| 36 | OG | SER C | 8 | 9.751 | 35.407 | 37.345 | 1.00 | 89.05 |
| 37 | C | SER C | 8 | 11.625 | 35.174 | 35.091 | 1.00 | 91.19 |
| 38 | O | SER C | 8 | 10.978 | 35.870 | 34.308 | 1.00 | 91.19 |
| 39 | N | LEU C | 9 | 12.047 | 33.946 | 34.794 | 1.00 | 76.39 |
| 40 | CA | LEU C | 9 | 11.750 | 33.300 | 33.511 | 1.00 | 76.39 |
| 41 | CB | LEU C | 9 | 13.016 | 33.111 | 32.687 | 1.00 | 48.15 |
| 42 | CG | LEU C | 9 | 13.863 | 34.301 | 32.245 | 1.00 | 48.15 |
| 43 | CD1 | LEU C | 9 | 14.684 | 33.924 | 31.048 | 1.00 | 48.15 |
| 44 | CD2 | LEU C | 9 | 12.964 | 35.448 | 31.863 | 1.00 | 48.15 |
| 45 | C | LEU C | 9 | 11.124 | 31.922 | 33.685 | 1.00 | 76.39 |
| 46 | O | LEU C | 9 | 11.321 | 31.262 | 34.690 | 1.00 | 76.39 |
| 47 | N | ASN C | 10 | 10.380 | 31.476 | 32.687 | 1.00 | 56.03 |
| 48 | CA | ASN C | 10 | 9.756 | 30.161 | 32.739 | 1.00 | 56.03 |
| 49 | CB | ASN C | 10 | 8.459 | 30.216 | 33.531 | 1.00 | 97.06 |
| 50 | CG | ASN C | 10 | 7.912 | 28.844 | 33.807 | 1.00 | 97.06 |
| 51 | OD1 | ASN C | 10 | 8.527 | 28.062 | 34.532 | 1.00 | 97.06 |
| 52 | ND2 | ASN C | 10 | 6.764 | 28.528 | 33.218 | 1.00 | 97.06 |
| 53 | C | ASN C | 10 | 9.460 | 29.670 | 31.333 | 1.00 | 56.03 |
| 54 | O | ASN C | 10 | 8.594 | 30.226 | 30.649 | 1.00 | 56.03 |
| 55 | N | PRO C | 11 | 10.173 | 28.619 | 30.873 | 1.00 | 62.47 |
| 56 | CD | PRO C | 11 | 10.022 | 28.168 | 29.482 | 1.00 | 141.22 |
| 57 | CA | PRO C | 11 | 11.225 | 27.865 | 31.546 | 1.00 | 62.47 |
| 58 | CB | PRO C | 11 | 11.726 | 26.936 | 30.444 | 1.00 | 141.22 |
| 59 | CG | PRO C | 11 | 10.542 | 26.774 | 29.564 | 1.00 | 141.22 |
| 60 | C | PRO C | 11 | 12.362 | 28.734 | 32.097 | 1.00 | 62.47 |
| 61 | O | PRO C | 11 | 12.512 | 29.887 | 31.703 | 1.00 | 62.47 |
| 62 | N | PRO C | 12 | 13.197 | 28.186 | 33.000 | 1.00 | 68.33 |
| 63 | CD | PRO C | 12 | 13.127 | 26.826 | 33.565 | 1.00 | 71.60 |
| 64 | CA | PRO C | 12 | 14.315 | 28.913 | 33.606 | 1.00 | 68.33 |
| 65 | CB | PRO C | 12 | 14.839 | 27.958 | 34.664 | 1.00 | 71.60 |
| 66 | CG | PRO C | 12 | 13.707 | 27.044 | 34.925 | 1.00 | 71.60 |
| 67 | C | PRO C | 12 | 15.383 | 29.190 | 32.567 | 1.00 | 68.33 |
| 68 | O | PRO C | 12 | 16.176 | 30.127 | 32.696 | 1.00 | 68.33 |
| 69 | N | TRP C | 13 | 15.395 | 28.352 | 31.538 | 1.00 | 58.74 |
| 70 | CA | TRP C | 13 | 16.378 | 28.444 | 30.466 | 1.00 | 58.74 |
| 71 | CB | TRP C | 13 | 16.076 | 27.401 | 29.401 | 1.00 | 68.19 |
| 72 | CG | TRP C | 13 | 15.812 | 26.077 | 29.969 | 1.00 | 68.19 |
| 73 | CD2 | TRP C | 13 | 16.476 | 25.473 | 31.064 | 1.00 | 68.19 |
| 74 | CE2 | TRP C | 13 | 15.848 | 24.241 | 31.307 | 1.00 | 68.19 |
| 75 | CE3 | TRP C | 13 | 17.547 | 25.852 | 31.873 | 1.00 | 68.19 |
| 76 | CD1 | TRP C | 13 | 14.844 | 25.220 | 29.538 | 1.00 | 68.19 |
| 77 | NE1 | TRP C | 13 | 14.848 | 24.114 | 30.391 | 1.00 | 68.19 |
| 78 | CZ2 | TRP C | 13 | 16.252 | 23.380 | 32.324 | 1.00 | 68.19 |
| 79 | CZ3 | TRP C | 13 | 17.950 | 24.993 | 32.892 | 1.00 | 68.19 |
| 80 | CH2 | TRP C | 13 | 17.300 | 23.771 | 33.107 | 1.00 | 68.19 |
| 81 | C | TRP C | 13 | 16.409 | 29.810 | 29.826 | 1.00 | 58.74 |
| 82 | O | TRP C | 13 | 15.409 | 30.264 | 29.288 | 1.00 | 58.74 |
| 83 | N | ASN C | 14 | 17.570 | 30.454 | 29.879 | 1.00 | 57.67 |
| 84 | CA | ASN C | 14 | 17.729 | 31.775 | 29.295 | 1.00 | 57.67 |
| 85 | CB | ASN C | 14 | 18.371 | 32.746 | 30.304 | 1.00 | 148.07 |
| 86 | CG | ASN C | 14 | 19.809 | 32.414 | 30.614 | 1.00 | 148.07 |
| 87 | OD1 | ASN C | 14 | 20.127 | 31.304 | 31.041 | 1.00 | 148.07 |
| 88 | ND2 | ASN C | 14 | 20.692 | 33.383 | 30.408 | 1.00 | 148.07 |
| 89 | C | ASN C | 14 | 18.508 | 31.761 | 27.990 | 1.00 | 57.67 |
| 90 | O | ASN C | 14 | 18.992 | 32.785 | 27.550 | 1.00 | 57.67 |
| 91 | N | ARG C | 15 | 18.645 | 30.590 | 27.378 | 1.00 | 58.44 |
| 92 | CA | ARG C | 15 | 19.311 | 30.455 | 26.078 | 1.00 | 58.44 |
| 93 | CB | ARG C | 15 | 20.634 | 29.728 | 26.174 | 1.00 | 68.23 |
| 94 | CG | ARG C | 15 | 21.469 | 30.131 | 27.329 | 1.00 | 68.23 |
| 95 | CD | ARG C | 15 | 22.779 | 29.404 | 27.261 | 1.00 | 68.23 |
| 96 | NE | ARG C | 15 | 23.607 | 29.885 | 26.172 | 1.00 | 68.23 |
| 97 | CZ | ARG C | 15 | 24.492 | 29.119 | 25.560 | 1.00 | 68.23 |
| 98 | NH1 | ARG C | 15 | 24.614 | 27.865 | 25.950 | 1.00 | 68.23 |
| 99 | NH2 | ARG C | 15 | 25.267 | 29.599 | 24.589 | 1.00 | 68.23 |
| 100 | C | ARG C | 15 | 18.345 | 29.540 | 25.394 | 1.00 | 58.44 |
| 101 | O | ARG C | 15 | 18.206 | 28.379 | 25.805 | 1.00 | 58.44 |
| 102 | N | ILE C | 16 | 17.648 | 30.048 | 24.386 | 1.00 | 56.07 |
| 103 | CA | ILE C | 16 | 16.691 | 29.214 | 23.693 | 1.00 | 56.07 |
| 104 | CB | ILE C | 16 | 15.279 | 29.668 | 23.944 | 1.00 | 49.05 |
| 105 | CG2 | ILE C | 16 | 14.939 | 29.490 | 25.385 | 1.00 | 49.05 |
| 106 | CG1 | ILE C | 16 | 15.128 | 31.116 | 23.520 | 1.00 | 49.05 |
| 107 | CD1 | ILE C | 16 | 13.760 | 31.675 | 23.801 | 1.00 | 49.05 |
| 108 | C | ILE C | 16 | 16.889 | 29.154 | 22.201 | 1.00 | 56.07 |
| 109 | O | ILE C | 16 | 17.607 | 29.956 | 21.610 | 1.00 | 56.07 |
| 110 | N | PHE C | 17 | 16.221 | 28.178 | 21.608 | 1.00 | 80.97 |
| 111 | CA | PHE C | 17 | 16.247 | 27.906 | 20.188 | 1.00 | 80.97 |
| 112 | CB | PHE C | 17 | 15.846 | 26.458 | 19.984 | 1.00 | 52.57 |
| 113 | CG | PHE C | 17 | 16.996 | 25.503 | 19.972 | 1.00 | 52.57 |
| 114 | CD1 | PHE C | 17 | 16.878 | 24.248 | 20.554 | 1.00 | 52.57 |
| 115 | CD2 | PHE C | 17 | 18.173 | 25.830 | 19.278 | 1.00 | 52.57 |
| 116 | CE1 | PHE C | 17 | 17.897 | 23.329 | 20.455 | 1.00 | 52.57 |
| 117 | CE2 | PHE C | 17 | 19.207 | 24.912 | 19.167 | 1.00 | 52.57 |
| 118 | CZ | PHE C | 17 | 19.063 | 23.648 | 19.759 | 1.00 | 52.57 |
| 119 | C | PHE C | 17 | 15.251 | 28.793 | 19.468 | 1.00 | 80.97 |
| 120 | O | PHE C | 17 | 14.320 | 29.320 | 20.074 | 1.00 | 80.97 |
| 121 | N | LYS C | 18 | 15.429 | 28.937 | 18.161 | 1.00 | 59.00 |
| 122 | CA | LYS C | 18 | 14.529 | 29.761 | 17.349 | 1.00 | 59.00 |
| 123 | CB | LYS C | 18 | 15.065 | 29.846 | 15.921 | 1.00 | 195.91 |
| 124 | CG | LYS C | 18 | 14.313 | 30.790 | 15.003 | 1.00 | 195.91 |
| 125 | CD | LYS C | 18 | 15.142 | 31.059 | 13.761 | 1.00 | 195.91 |
| 126 | CE | LYS C | 18 | 14.441 | 32.000 | 12.803 | 1.00 | 195.91 |
| 127 | NZ | LYS C | 18 | 13.160 | 31.413 | 12.321 | 1.00 | 195.91 |
| 128 | C | LYS C | 18 | 13.123 | 29.162 | 17.349 | 1.00 | 59.00 |
| 129 | O | LYS C | 18 | 12.937 | 27.974 | 17.129 | 1.00 | 59.00 |
| 130 | N | GLY C | 19 | 12.122 | 29.973 | 17.630 | 1.00 | 76.33 |
| 131 | CA | GLY C | 19 | 10.774 | 29.457 | 17.582 | 1.00 | 76.33 |
| 132 | C | GLY C | 19 | 10.178 | 28.991 | 18.886 | 1.00 | 76.33 |
| 133 | O | GLY C | 19 | 8.971 | 28.747 | 18.970 | 1.00 | 76.33 |
| 134 | N | GLU C | 20 | 10.998 | 28.857 | 19.916 | 1.00 | 72.26 |
| 135 | CA | GLU C | 20 | 10.460 | 28.427 | 21.211 | 1.00 | 72.26 |
| 136 | CB | GLU C | 20 | 11.590 | 27.847 | 22.059 | 1.00 | 102.87 |
| 137 | CG | GLU C | 20 | 12.410 | 26.815 | 21.296 | 1.00 | 102.87 |
| 138 | CD | GLU C | 20 | 13.457 | 26.129 | 22.152 | 1.00 | 102.87 |
| 139 | OE1 | GLU C | 20 | 14.291 | 26.830 | 22.758 | 1.00 | 102.87 |
| 140 | OE2 | GLU C | 20 | 13.452 | 24.884 | 22.210 | 1.00 | 102.87 |
| 141 | C | GLU C | 20 | 9.739 | 29.579 | 21.956 | 1.00 | 72.26 |
| 142 | O | GLU C | 20 | 9.803 | 30.730 | 21.525 | 1.00 | 72.26 |
| 143 | N | ASN C | 21 | 9.030 | 29.264 | 23.040 | 1.00 | 57.87 |
| 144 | CA | ASN C | 21 | 8.336 | 30.295 | 23.787 | 1.00 | 57.87 |
| 145 | CB | ASN C | 21 | 6.839 | 30.041 | 23.853 | 1.00 | 107.77 |
| 146 | CG | ASN C | 21 | 6.273 | 29.544 | 22.563 | 1.00 | 107.77 |
| 147 | OD1 | ASN C | 21 | 6.639 | 30.019 | 21.477 | 1.00 | 107.77 |
| 148 | ND2 | ASN C | 21 | 5.353 | 28.591 | 22.690 | 1.00 | 107.77 |
| 149 | C | ASN C | 21 | 8.841 | 30.401 | 25.220 | 1.00 | 57.87 |
| 150 | O | ASN C | 21 | 9.136 | 29.391 | 25.859 | 1.00 | 57.87 |
| 151 | N | VAL C | 22 | 8.921 | 31.625 | 25.735 | 1.00 | 64.18 |
| 152 | CA | VAL C | 22 | 9.364 | 31.858 | 27.099 | 1.00 | 64.18 |
| 153 | CB | VAL C | 22 | 10.797 | 32.278 | 27.139 | 1.00 | 42.75 |
| 154 | CG1 | VAL C | 22 | 10.981 | 33.583 | 26.376 | 1.00 | 42.75 |
| 155 | CG2 | VAL C | 22 | 11.231 | 32.452 | 28.585 | 1.00 | 42.75 |
| 156 | C | VAL C | 22 | 8.542 | 32.997 | 27.677 | 1.00 | 64.18 |
| 157 | O | VAL C | 22 | 8.115 | 33.897 | 26.936 | 1.00 | 64.18 |
| 158 | N | THR C | 23 | 8.347 | 32.977 | 28.998 | 1.00 | 75.81 |
| 159 | CA | THR C | 23 | 7.534 | 33.987 | 29.693 | 1.00 | 75.81 |
| 160 | CB | THR C | 23 | 6.369 | 33.301 | 30.399 | 1.00 | 170.16 |
| 161 | OG1 | THR C | 23 | 5.651 | 32.492 | 29.459 | 1.00 | 170.16 |
| 162 | CG2 | THR C | 23 | 5.442 | 34.327 | 31.005 | 1.00 | 170.16 |
| 163 | C | THR C | 23 | 8.328 | 34.776 | 30.730 | 1.00 | 75.81 |
| 164 | O | THR C | 23 | 8.978 | 34.183 | 31.572 | 1.00 | 75.81 |
| 165 | N | LEU C | 24 | 8.292 | 36.101 | 30.684 | 1.00 | 82.13 |
| 166 | CA | LEU C | 24 | 9.071 | 36.861 | 31.656 | 1.00 | 82.13 |
| 167 | CB | LEU C | 24 | 9.899 | 37.962 | 30.995 | 1.00 | 55.82 |
| 168 | CG | LEU C | 24 | 10.586 | 37.719 | 29.653 | 1.00 | 55.82 |
| 169 | CD1 | LEU C | 24 | 11.621 | 38.790 | 29.358 | 1.00 | 55.82 |
| 170 | CD2 | LEU C | 24 | 11.241 | 36.405 | 29.664 | 1.00 | 55.82 |
| 171 | C | LEU C | 24 | 8.182 | 37.506 | 32.677 | 1.00 | 82.13 |
| 172 | O | LEU C | 24 | 7.526 | 38.505 | 32.391 | 1.00 | 82.13 |
| 173 | N | THR C | 25 | 8.184 | 36.967 | 33.888 | 1.00 | 46.04 |
| 174 | CA | THR C | 25 | 7.333 | 37.517 | 34.921 | 1.00 | 46.04 |
| 175 | CB | THR C | 25 | 6.859 | 36.406 | 35.852 | 1.00 | 88.77 |
| 176 | OG1 | THR C | 25 | 6.235 | 35.384 | 35.064 | 1.00 | 88.77 |
| 177 | CG2 | THR C | 25 | 5.846 | 36.939 | 36.851 | 1.00 | 88.77 |
| 178 | C | THR C | 25 | 8.047 | 38.614 | 35.693 | 1.00 | 46.04 |
| 179 | O | THR C | 25 | 9.225 | 38.493 | 36.009 | 1.00 | 46.04 |
| 180 | N | CYS C | 26 | 7.360 | 39.719 | 35.962 | 1.00 | 99.22 |
| 181 | CA | CYS C | 26 | 7.988 | 40.779 | 36.730 | 1.00 | 99.22 |
| 182 | C | CYS C | 26 | 7.833 | 40.454 | 38.201 | 1.00 | 99.22 |
| 183 | O | CYS C | 26 | 6.787 | 39.984 | 38.644 | 1.00 | 99.22 |
| 184 | CB | CYS C | 26 | 7.353 | 42.132 | 36.440 | 1.00 | 145.11 |
| 185 | SG | CYS C | 26 | 8.267 | 43.513 | 37.198 | 1.00 | 145.11 |
| 186 | N | ASN C | 27 | 8.897 | 40.697 | 38.944 | 1.00 | 197.95 |
| 187 | CA | ASN C | 27 | 8.936 | 40.461 | 40.370 | 1.00 | 197.95 |
| 188 | CB | ASN C | 27 | 9.427 | 41.723 | 41.048 | 1.00 | 249.36 |
| 189 | CG | ASN C | 27 | 9.941 | 41.459 | 42.424 | 1.00 | 249.36 |
| 190 | OD1 | ASN C | 27 | 10.558 | 40.419 | 42.666 | 1.00 | 249.36 |
| 191 | ND2 | ASN C | 27 | 9.710 | 42.399 | 43.346 | 1.00 | 249.36 |
| 192 | C | ASN C | 27 | 7.618 | 40.026 | 41.003 | 1.00 | 197.95 |
| 193 | O | ASN C | 27 | 6.829 | 40.859 | 41.440 | 1.00 | 197.95 |
| 194 | N | GLY C | 28 | 7.392 | 38.719 | 41.065 | 1.00 | 214.74 |
| 195 | CA | GLY C | 28 | 6.162 | 38.203 | 41.644 | 1.00 | 214.74 |
| 196 | C | GLY C | 28 | 6.121 | 36.711 | 41.398 | 1.00 | 214.74 |
| 197 | O | GLY C | 28 | 6.177 | 36.276 | 40.255 | 1.00 | 214.74 |
| 198 | N | ASN C | 29 | 6.006 | 35.922 | 42.456 | 1.00 | 249.28 |
| 199 | CA | ASN C | 29 | 6.011 | 34.476 | 42.322 | 1.00 | 249.28 |
| 200 | CB | ASN C | 29 | 6.332 | 33.825 | 43.676 | 1.00 | 216.11 |
| 201 | CG | ASN C | 29 | 6.655 | 32.353 | 43.552 | 1.00 | 216.11 |
| 202 | OD1 | ASN C | 29 | 6.877 | 31.849 | 42.450 | 1.00 | 216.11 |
| 203 | ND2 | ASN C | 29 | 6.701 | 31.657 | 44.681 | 1.00 | 216.11 |
| 204 | C | ASN C | 29 | 4.731 | 33.880 | 41.751 | 1.00 | 249.28 |
| 205 | O | ASN C | 29 | 4.781 | 33.119 | 40.788 | 1.00 | 249.28 |
| 206 | N | ASN C | 30 | 3.584 | 34.225 | 42.328 | 1.00 | 235.48 |
| 207 | CA | ASN C | 30 | 2.325 | 33.663 | 41.851 | 1.00 | 235.48 |
| 208 | CB | ASN C | 30 | 1.763 | 32.685 | 42.889 | 1.00 | 219.86 |
| 209 | CG | ASN C | 30 | 2.660 | 31.487 | 43.106 | 1.00 | 219.86 |
| 210 | OD1 | ASN C | 30 | 3.006 | 31.152 | 44.240 | 1.00 | 219.86 |
| 211 | ND2 | ASN C | 30 | 3.040 | 30.831 | 42.019 | 1.00 | 219.86 |
| 212 | C | ASN C | 30 | 1.251 | 34.682 | 41.498 | 1.00 | 235.48 |
| 213 | O | ASN C | 30 | 0.931 | 34.878 | 40.325 | 1.00 | 235.48 |
| 214 | N | PHE C | 31 | 0.690 | 35.329 | 42.515 | 1.00 | 241.86 |
| 215 | CA | PHE C | 31 | −0.373 | 36.291 | 42.280 | 1.00 | 241.86 |
| 216 | CB | PHE C | 31 | −1.597 | 35.920 | 43.123 | 1.00 | 249.47 |
| 217 | CG | PHE C | 31 | −2.076 | 34.504 | 42.908 | 1.00 | 249.47 |
| 218 | CD1 | PHE C | 31 | −1.432 | 33.431 | 43.523 | 1.00 | 249.47 |
| 219 | CD2 | PHE C | 31 | −3.154 | 34.240 | 42.066 | 1.00 | 249.47 |
| 220 | CE1 | PHE C | 31 | −1.858 | 32.115 | 43.307 | 1.00 | 249.47 |
| 221 | CE2 | PHE C | 31 | −3.588 | 32.927 | 41.843 | 1.00 | 249.47 |
| 222 | CZ | PHE C | 31 | −2.936 | 31.863 | 42.463 | 1.00 | 249.47 |
| 223 | C | PHE C | 31 | 0.022 | 37.743 | 42.516 | 1.00 | 241.86 |
| 224 | O | PHE C | 31 | 0.520 | 38.109 | 43.587 | 1.00 | 241.86 |
| 225 | N | PHE C | 32 | −0.212 | 38.559 | 41.489 | 1.00 | 249.62 |
| 226 | CA | PHE C | 32 | 0.108 | 39.985 | 41.512 | 1.00 | 249.62 |
| 227 | CB | PHE C | 32 | 1.132 | 40.302 | 40.423 | 1.00 | 249.66 |
| 228 | CG | PHE C | 32 | 1.755 | 41.655 | 40.560 | 1.00 | 249.66 |
| 229 | CD1 | PHE C | 32 | 2.582 | 41.907 | 41.614 | 1.00 | 249.66 |
| 230 | CD2 | PHE C | 32 | 1.510 | 42.675 | 39.643 | 1.00 | 249.66 |
| 231 | CE1 | PHE C | 32 | 3.147 | 43.108 | 41.753 | 1.00 | 249.66 |
| 232 | CE2 | PHE C | 32 | 2.093 | 43.918 | 39.791 | 1.00 | 249.66 |
| 233 | CZ | PHE C | 32 | 2.900 | 44.146 | 40.828 | 1.00 | 249.66 |
| 234 | C | PHE C | 32 | −1.151 | 40.815 | 41.269 | 1.00 | 249.62 |
| 235 | O | PHE C | 32 | −2.197 | 40.259 | 40.930 | 1.00 | 249.62 |
| 236 | N | GLU C | 33 | −1.054 | 42.139 | 41.416 | 1.00 | 249.77 |
| 237 | CA | GLU C | 33 | −2.224 | 42.994 | 41.200 | 1.00 | 249.77 |
| 238 | CB | GLU C | 33 | −2.704 | 43.604 | 42.503 | 1.00 | 249.65 |
| 239 | CG | GLU C | 33 | −4.023 | 44.344 | 42.358 | 1.00 | 249.65 |
| 240 | CD | GLU C | 33 | −5.159 | 43.406 | 42.025 | 1.00 | 249.65 |
| 241 | OE1 | GLU C | 33 | −5.159 | 42.298 | 42.562 | 1.00 | 249.65 |
| 242 | OE2 | GLU C | 33 | −6.051 | 43.779 | 41.239 | 1.00 | 249.65 |
| 243 | C | GLU C | 33 | −2.110 | 44.128 | 40.194 | 1.00 | 249.77 |
| 244 | O | GLU C | 33 | −2.952 | 44.257 | 39.301 | 1.00 | 249.77 |
| 245 | N | VAL C | 34 | −1.107 | 44.982 | 40.365 | 1.00 | 243.09 |
| 246 | CA | VAL C | 34 | −0.949 | 46.113 | 39.471 | 1.00 | 243.09 |
| 247 | CB | VAL C | 34 | 0.351 | 46.880 | 39.775 | 1.00 | 249.25 |
| 248 | CG1 | VAL C | 34 | 0.508 | 48.060 | 38.826 | 1.00 | 249.25 |
| 249 | CG2 | VAL C | 34 | 0.302 | 47.386 | 41.184 | 1.00 | 249.25 |
| 250 | C | VAL C | 34 | −0.990 | 45.721 | 38.002 | 1.00 | 243.09 |
| 251 | O | VAL C | 34 | −0.603 | 44.616 | 37.615 | 1.00 | 243.09 |
| 252 | N | SER C | 35 | −1.494 | 46.644 | 37.196 | 1.00 | 146.24 |
| 253 | CA | SER C | 35 | −1.605 | 46.453 | 35.764 | 1.00 | 146.24 |
| 254 | CB | SER C | 35 | −3.021 | 46.778 | 35.290 | 1.00 | 174.88 |
| 255 | OG | SER C | 35 | −3.296 | 48.166 | 35.411 | 1.00 | 174.88 |
| 256 | C | SER C | 35 | −0.617 | 47.400 | 35.103 | 1.00 | 146.24 |
| 257 | O | SER C | 35 | −0.518 | 47.438 | 33.878 | 1.00 | 146.24 |
| 258 | N | SER C | 36 | 0.095 | 48.179 | 35.919 | 1.00 | 112.51 |
| 259 | CA | SER C | 36 | 1.091 | 49.114 | 35.408 | 1.00 | 112.51 |
| 260 | CB | SER C | 36 | 0.986 | 50.475 | 36.105 | 1.00 | 242.80 |
| 261 | OG | SER C | 36 | 1.420 | 50.408 | 37.452 | 1.00 | 242.80 |
| 262 | C | SER C | 36 | 2.486 | 48.535 | 35.635 | 1.00 | 112.51 |
| 263 | O | SER C | 36 | 3.088 | 48.707 | 36.700 | 1.00 | 112.51 |
| 264 | N | THR C | 37 | 2.985 | 47.834 | 34.620 | 1.00 | 147.41 |
| 265 | CA | THR C | 37 | 4.301 | 47.220 | 34.655 | 1.00 | 147.41 |
| 266 | CB | THR C | 37 | 4.185 | 45.679 | 34.635 | 1.00 | 242.04 |
| 267 | OG1 | THR C | 37 | 3.393 | 45.242 | 35.748 | 1.00 | 242.04 |
| 268 | CG2 | THR C | 37 | 5.553 | 45.039 | 34.720 | 1.00 | 242.04 |
| 269 | C | THR C | 37 | 5.004 | 47.708 | 33.399 | 1.00 | 147.41 |
| 270 | O | THR C | 37 | 4.382 | 47.834 | 32.345 | 1.00 | 147.41 |
| 271 | N | LYS C | 38 | 6.289 | 48.009 | 33.512 | 1.00 | 114.65 |
| 272 | CA | LYS C | 38 | 7.046 | 48.490 | 32.361 | 1.00 | 114.65 |
| 273 | CB | LYS C | 38 | 7.794 | 49.755 | 32.733 | 1.00 | 121.59 |
| 274 | CG | LYS C | 38 | 6.890 | 50.832 | 33.262 | 1.00 | 121.59 |
| 275 | CD | LYS C | 38 | 7.679 | 52.074 | 33.632 | 1.00 | 121.59 |
| 276 | CE | LYS C | 38 | 6.757 | 53.183 | 34.088 | 1.00 | 121.59 |
| 277 | NZ | LYS C | 38 | 7.518 | 54.413 | 34.415 | 1.00 | 121.59 |
| 278 | C | LYS C | 38 | 8.045 | 47.459 | 31.856 | 1.00 | 114.65 |
| 279 | O | LYS C | 38 | 8.640 | 46.745 | 32.652 | 1.00 | 114.65 |
| 280 | N | TRP C | 39 | 8.222 | 47.373 | 30.538 | 1.00 | 83.37 |
| 281 | CA | TRP C | 39 | 9.182 | 46.434 | 29.954 | 1.00 | 83.37 |
| 282 | CB | TRP C | 39 | 8.477 | 45.308 | 29.202 | 1.00 | 59.20 |
| 283 | CG | TRP C | 39 | 7.651 | 44.439 | 30.060 | 1.00 | 59.20 |
| 284 | CD2 | TRP C | 39 | 8.116 | 43.493 | 31.026 | 1.00 | 59.20 |
| 285 | CE2 | TRP C | 39 | 6.973 | 42.881 | 31.590 | 1.00 | 59.20 |
| 286 | CE3 | TRP C | 39 | 9.391 | 43.100 | 31.474 | 1.00 | 59.20 |
| 287 | CD1 | TRP C | 39 | 6.298 | 44.369 | 30.071 | 1.00 | 59.20 |
| 288 | NE1 | TRP C | 39 | 5.881 | 43.435 | 30.990 | 1.00 | 59.20 |
| 289 | CZ2 | TRP C | 39 | 7.061 | 41.892 | 32.576 | 1.00 | 59.20 |
| 290 | CZ3 | TRP C | 39 | 9.476 | 42.119 | 32.456 | 1.00 | 59.20 |
| 291 | CH2 | TRP C | 39 | 8.312 | 41.524 | 32.998 | 1.00 | 59.20 |
| 292 | C | TRP C | 39 | 10.086 | 47.179 | 28.990 | 1.00 | 83.37 |
| 293 | O | TRP C | 39 | 9.612 | 47.932 | 28.144 | 1.00 | 83.37 |
| 294 | N | PHE C | 40 | 11.387 | 46.963 | 29.116 | 1.00 | 81.86 |
| 295 | CA | PHE C | 40 | 12.330 | 47.639 | 28.248 | 1.00 | 81.86 |
| 296 | CB | PHE C | 40 | 13.204 | 48.591 | 29.062 | 1.00 | 132.74 |
| 297 | CG | PHE C | 40 | 12.433 | 49.601 | 29.852 | 1.00 | 132.74 |
| 298 | CD1 | PHE C | 40 | 11.846 | 49.258 | 31.063 | 1.00 | 132.74 |
| 299 | CD2 | PHE C | 40 | 12.305 | 50.903 | 29.393 | 1.00 | 132.74 |
| 300 | CE1 | PHE C | 40 | 11.141 | 50.201 | 31.812 | 1.00 | 132.74 |
| 301 | CE2 | PHE C | 40 | 11.603 | 51.853 | 30.130 | 1.00 | 132.74 |
| 302 | CZ | PHE C | 40 | 11.020 | 51.501 | 31.344 | 1.00 | 132.74 |
| 303 | C | PHE C | 40 | 13.225 | 46.677 | 27.474 | 1.00 | 81.86 |
| 304 | O | PHE C | 40 | 14.321 | 46.333 | 27.917 | 1.00 | 81.86 |
| 305 | N | HIS C | 41 | 12.761 | 46.239 | 26.314 | 1.00 | 70.61 |
| 306 | CA | HIS C | 41 | 13.552 | 45.334 | 25.490 | 1.00 | 70.61 |
| 307 | CB | HIS C | 41 | 12.633 | 44.671 | 24.470 | 1.00 | 75.99 |
| 308 | CG | HIS C | 41 | 13.339 | 43.759 | 23.528 | 1.00 | 75.99 |
| 309 | CD2 | HIS C | 41 | 13.192 | 43.567 | 22.198 | 1.00 | 75.99 |
| 310 | ND1 | HIS C | 41 | 14.327 | 42.893 | 23.933 | 1.00 | 75.99 |
| 311 | CE1 | HIS C | 41 | 14.765 | 42.207 | 22.892 | 1.00 | 75.99 |
| 312 | NE2 | HIS C | 41 | 14.093 | 42.598 | 21.826 | 1.00 | 75.99 |
| 313 | C | HIS C | 41 | 14.671 | 46.118 | 24.794 | 1.00 | 70.61 |
| 314 | O | HIS C | 41 | 14.408 | 46.922 | 23.918 | 1.00 | 70.61 |
| 315 | N | ASN C | 42 | 15.916 | 45.879 | 25.177 | 1.00 | 90.99 |
| 316 | CA | ASN C | 42 | 17.063 | 46.600 | 24.615 | 1.00 | 90.99 |
| 317 | CB | ASN C | 42 | 17.150 | 46.463 | 23.085 | 1.00 | 90.93 |
| 318 | CG | ASN C | 42 | 17.611 | 45.087 | 22.641 | 1.00 | 90.93 |
| 319 | OD1 | ASN C | 42 | 17.149 | 44.097 | 23.186 | 1.00 | 90.93 |
| 320 | ND2 | ASN C | 42 | 18.495 | 45.007 | 21.649 | 1.00 | 90.93 |
| 321 | C | ASN C | 42 | 16.966 | 48.077 | 24.971 | 1.00 | 90.99 |
| 322 | O | ASN C | 42 | 17.474 | 48.926 | 24.246 | 1.00 | 90.99 |
| 323 | N | GLY C | 43 | 16.315 | 48.394 | 26.086 | 1.00 | 101.51 |
| 324 | CA | GLY C | 43 | 16.177 | 49.792 | 26.478 | 1.00 | 101.51 |
| 325 | C | GLY C | 43 | 14.889 | 50.456 | 25.997 | 1.00 | 101.51 |
| 326 | O | GLY C | 43 | 14.265 | 51.235 | 26.721 | 1.00 | 101.51 |
| 327 | N | SER C | 44 | 14.492 | 50.140 | 24.769 | 1.00 | 159.89 |
| 328 | CA | SER C | 44 | 13.276 | 50.686 | 24.182 | 1.00 | 159.89 |
| 329 | CB | SER C | 44 | 13.183 | 50.282 | 22.705 | 1.00 | 153.29 |
| 330 | OG | SER C | 44 | 14.375 | 50.612 | 22.007 | 1.00 | 153.29 |
| 331 | C | SER C | 44 | 12.046 | 50.168 | 24.931 | 1.00 | 159.89 |
| 332 | O | SER C | 44 | 11.886 | 48.962 | 25.114 | 1.00 | 159.89 |
| 333 | N | LEU C | 45 | 11.179 | 51.076 | 25.368 | 1.00 | 127.30 |
| 334 | CA | LEU C | 45 | 9.969 | 50.682 | 26.091 | 1.00 | 127.30 |
| 335 | CB | LEU C | 45 | 9.143 | 51.925 | 26.443 | 1.00 | 113.27 |
| 336 | CG | LEU C | 45 | 7.855 | 51.691 | 27.238 | 1.00 | 113.27 |
| 337 | CD1 | LEU C | 45 | 8.167 | 50.902 | 28.502 | 1.00 | 113.27 |
| 338 | CD2 | LEU C | 45 | 7.210 | 53.024 | 27.593 | 1.00 | 113.27 |
| 339 | C | LEU C | 45 | 9.126 | 49.705 | 25.261 | 1.00 | 127.30 |
| 340 | O | LEU C | 45 | 9.084 | 49.805 | 24.039 | 1.00 | 127.30 |
| 341 | N | SER C | 46 | 8.458 | 48.758 | 25.915 | 1.00 | 104.59 |
| 342 | CA | SER C | 46 | 7.636 | 47.784 | 25.206 | 1.00 | 104.59 |
| 343 | CB | SER C | 46 | 7.802 | 46.400 | 25.829 | 1.00 | 120.90 |
| 344 | OG | SER C | 46 | 7.052 | 45.423 | 25.111 | 1.00 | 120.90 |
| 345 | C | SER C | 46 | 0.194 | 48.226 | 25.309 | 1.00 | 104.59 |
| 346 | O | SER C | 46 | 5.867 | 49.072 | 26.127 | 1.00 | 104.59 |
| 347 | N | GLU C | 47 | 5.320 | 47.643 | 24.495 | 1.00 | 161.06 |
| 348 | CA | GLU C | 47 | 3.919 | 48.057 | 24.551 | 1.00 | 161.06 |
| 349 | CB | GLU C | 47 | 3.295 | 48.100 | 23.152 | 1.00 | 249.30 |
| 350 | CG | GLU C | 47 | 4.218 | 48.523 | 22.010 | 1.00 | 249.30 |
| 351 | CD | GLU C | 47 | 3.700 | 48.108 | 20.617 | 1.00 | 249.30 |
| 352 | OE1 | GLU C | 47 | 4.006 | 46.969 | 20.155 | 1.00 | 249.30 |
| 353 | OE2 | GLU C | 47 | 2.988 | 48.918 | 19.972 | 1.00 | 249.30 |
| 354 | C | GLU C | 47 | 3.070 | 47.171 | 25.505 | 1.00 | 161.06 |
| 355 | O | GLU C | 47 | 1.875 | 47.409 | 25.648 | 1.00 | 161.06 |
| 356 | N | GLU C | 48 | 3.655 | 46.147 | 26.142 | 1.00 | 104.22 |
| 357 | CA | GLU C | 48 | 2.859 | 45.337 | 27.077 | 1.00 | 104.22 |
| 358 | CB | GLU C | 48 | 3.427 | 43.913 | 27.244 | 1.00 | 144.62 |
| 359 | CG | GLU C | 48 | 2.742 | 43.070 | 28.349 | 1.00 | 144.62 |
| 360 | CD | GLU C | 48 | 1.288 | 42.704 | 28.064 | 1.00 | 144.62 |
| 361 | OE1 | GLU C | 48 | 1.034 | 41.897 | 27.140 | 1.00 | 144.62 |
| 362 | OE2 | GLU C | 48 | 0.396 | 43.221 | 28.775 | 1.00 | 144.62 |
| 363 | C | GLU C | 48 | 2.829 | 46.060 | 28.424 | 1.00 | 104.22 |
| 364 | O | GLU C | 48 | 3.708 | 46.868 | 28.724 | 1.00 | 104.22 |
| 365 | N | THR C | 49 | 1.813 | 45.771 | 29.229 | 1.00 | 87.76 |
| 366 | CA | THR C | 49 | 1.677 | 46.399 | 30.529 | 1.00 | 87.76 |
| 367 | CB | THR C | 49 | 0.505 | 47.406 | 30.547 | 1.00 | 167.47 |
| 368 | OG1 | THR C | 49 | −0.713 | 46.751 | 30.168 | 1.00 | 167.47 |
| 369 | CG2 | THR C | 49 | 0.788 | 48.546 | 29.576 | 1.00 | 167.47 |
| 370 | C | THR C | 49 | 1.461 | 45.342 | 31.601 | 1.00 | 87.76 |
| 371 | O | THR C | 49 | 1.832 | 45.537 | 32.751 | 1.00 | 87.76 |
| 372 | N | ASN C | 50 | 0.872 | 44.210 | 31.227 | 1.00 | 92.41 |
| 373 | CA | ASN C | 50 | 0.637 | 43.123 | 32.180 | 1.00 | 92.41 |
| 374 | CB | ASN C | 50 | 0.006 | 41.921 | 31.455 | 1.00 | 211.05 |
| 375 | CG | ASN C | 50 | −0.583 | 40.901 | 32.411 | 1.00 | 211.05 |
| 376 | OD1 | ASN C | 50 | −0.245 | 40.896 | 33.593 | 1.00 | 211.05 |
| 377 | ND2 | ASN C | 50 | −1.449 | 40.025 | 31.907 | 1.00 | 211.05 |
| 378 | C | ASN C | 50 | 2.006 | 42.743 | 32.772 | 1.00 | 92.41 |
| 379 | O | ASN C | 50 | 3.035 | 42.908 | 32.125 | 1.00 | 92.41 |
| 380 | N | SER C | 51 | 2.026 | 42.252 | 34.005 | 1.00 | 91.81 |
| 381 | CA | SER C | 51 | 3.280 | 41.858 | 34.640 | 1.00 | 91.81 |
| 382 | CB | SER C | 51 | 3.042 | 41.518 | 36.117 | 1.00 | 188.83 |
| 383 | OG | SER C | 51 | 2.293 | 40.322 | 36.271 | 1.00 | 188.83 |
| 384 | C | SER C | 51 | 3.948 | 40.661 | 33.944 | 1.00 | 91.81 |
| 385 | O | SER C | 51 | 5.130 | 40.414 | 34.137 | 1.00 | 91.81 |
| 386 | N | SER C | 52 | 3.199 | 39.919 | 33.136 | 1.00 | 82.66 |
| 387 | CA | SER C | 52 | 3.750 | 38.764 | 32.450 | 1.00 | 82.66 |
| 388 | CB | SER C | 52 | 2.862 | 37.530 | 32.662 | 1.00 | 107.08 |
| 389 | OG | SER C | 52 | 2.845 | 37.147 | 34.025 | 1.00 | 107.08 |
| 390 | C | SER C | 52 | 3.860 | 39.064 | 30.976 | 1.00 | 82.66 |
| 391 | O | SER C | 52 | 2.866 | 39.155 | 30.271 | 1.00 | 82.66 |
| 392 | N | LEU C | 53 | 5.089 | 39.228 | 30.524 | 1.00 | 52.71 |
| 393 | CA | LEU C | 53 | 5.386 | 39.501 | 29.126 | 1.00 | 52.71 |
| 394 | CB | LEU C | 53 | 6.563 | 40.483 | 29.036 | 1.00 | 59.51 |
| 395 | CG | LEU C | 53 | 7.380 | 40.539 | 27.742 | 1.00 | 59.51 |
| 396 | CD1 | LEU C | 53 | 6.474 | 40.524 | 26.514 | 1.00 | 59.51 |
| 397 | CD2 | LEU C | 53 | 8.217 | 41.797 | 27.765 | 1.00 | 59.51 |
| 398 | C | LEU C | 53 | 5.741 | 38.215 | 28.378 | 1.00 | 52.71 |
| 399 | O | LEU C | 53 | 6.880 | 37.750 | 28.462 | 1.00 | 52.71 |
| 400 | N | ASN C | 54 | 4.794 | 37.650 | 27.631 | 1.00 | 78.83 |
| 401 | CA | ASN C | 54 | 5.073 | 36.425 | 26.889 | 1.00 | 78.83 |
| 402 | CB | ASN C | 54 | 3.777 | 35.731 | 26.511 | 1.00 | 114.28 |
| 403 | CG | ASN C | 54 | 3.093 | 35.117 | 27.699 | 1.00 | 114.28 |
| 404 | OD1 | ASN C | 54 | 3.685 | 34.315 | 28.415 | 1.00 | 114.28 |
| 405 | ND2 | ASN C | 54 | 1.842 | 35.488 | 27.922 | 1.00 | 114.28 |
| 406 | C | ASN C | 54 | 5.898 | 36.641 | 25.629 | 1.00 | 78.83 |
| 407 | O | ASN C | 54 | 5.983 | 37.745 | 25.099 | 1.00 | 78.83 |
| 408 | N | ILE C | 55 | 6.527 | 35.566 | 25.174 | 1.00 | 69.41 |
| 409 | CA | ILE C | 55 | 7.321 | 35.571 | 23.962 | 1.00 | 69.41 |
| 410 | CB | ILE C | 55 | 8.814 | 35.555 | 24.270 | 1.00 | 55.40 |
| 411 | CG2 | ILE C | 55 | 9.596 | 35.167 | 23.036 | 1.00 | 55.40 |
| 412 | CG1 | ILE C | 55 | 9.238 | 36.952 | 24.724 | 1.00 | 55.40 |
| 413 | CD1 | ILE C | 55 | 10.730 | 37.122 | 25.012 | 1.00 | 55.40 |
| 414 | C | ILE C | 55 | 6.935 | 34.320 | 23.210 | 1.00 | 69.41 |
| 415 | O | ILE C | 55 | 7.048 | 33.232 | 23.744 | 1.00 | 69.41 |
| 416 | N | VAL C | 56 | 6.442 | 34.473 | 21.989 | 1.00 | 107.00 |
| 417 | CA | VAL C | 56 | 6.046 | 33.317 | 21.199 | 1.00 | 107.00 |
| 418 | CB | VAL C | 56 | 4.721 | 33.566 | 20.504 | 1.00 | 128.23 |
| 419 | CG1 | VAL C | 56 | 4.126 | 32.254 | 20.058 | 1.00 | 128.23 |
| 420 | CG2 | VAL C | 56 | 3.772 | 34.277 | 21.453 | 1.00 | 128.23 |
| 421 | C | VAL C | 56 | 7.132 | 33.041 | 20.171 | 1.00 | 107.00 |
| 422 | O | VAL C | 56 | 8.236 | 33.546 | 20.317 | 1.00 | 107.00 |
| 423 | N | ASN C | 57 | 6.837 | 32.251 | 19.142 | 1.00 | 99.37 |
| 424 | CA | ASN C | 57 | 7.833 | 31.906 | 18.123 | 1.00 | 99.37 |
| 425 | CB | ASN C | 57 | 7.201 | 31.916 | 16.733 | 1.00 | 170.52 |
| 426 | CG | ASN C | 57 | 6.217 | 30.781 | 16.541 | 1.00 | 170.52 |
| 427 | OD1 | ASN C | 57 | 6.543 | 29.617 | 16.766 | 1.00 | 170.52 |
| 428 | ND2 | ASN C | 57 | 5.000 | 31.115 | 16.127 | 1.00 | 170.52 |
| 429 | C | ASN C | 57 | 9.053 | 32.828 | 18.157 | 1.00 | 99.37 |
| 430 | O | ASN C | 57 | 9.105 | 33.850 | 17.480 | 1.00 | 99.37 |
| 431 | N | ALA C | 58 | 10.033 | 32.443 | 18.966 | 1.00 | 78.85 |
| 432 | CA | ALA C | 58 | 11.241 | 33.220 | 19.162 | 1.00 | 78.85 |
| 433 | CB | ALA C | 58 | 12.180 | 32.478 | 20.085 | 1.00 | 109.58 |
| 434 | C | ALA C | 58 | 11.951 | 33.558 | 17.878 | 1.00 | 78.85 |
| 435 | O | ALA C | 58 | 12.358 | 32.681 | 17.139 | 1.00 | 78.85 |
| 436 | N | LYS C | 59 | 12.094 | 34.845 | 17.610 | 1.00 | 66.66 |
| 437 | CA | LYS C | 59 | 12.812 | 35.317 | 16.428 | 1.00 | 66.66 |
| 438 | CB | LYS C | 59 | 11.988 | 36.405 | 15.726 | 1.00 | 201.62 |
| 439 | CG | LYS C | 59 | 10.597 | 35.939 | 15.295 | 1.00 | 201.62 |
| 440 | CD | LYS C | 59 | 9.751 | 37.070 | 14.724 | 1.00 | 201.62 |
| 441 | CE | LYS C | 59 | 8.374 | 36.569 | 14.307 | 1.00 | 201.62 |
| 442 | NZ | LYS C | 59 | 7.518 | 37.663 | 13.775 | 1.00 | 201.62 |
| 443 | C | LYS C | 59 | 14.146 | 35.890 | 16.953 | 1.00 | 66.66 |
| 444 | O | LYS C | 59 | 14.194 | 36.455 | 18.055 | 1.00 | 66.66 |
| 445 | N | PHE C | 60 | 15.224 | 35.743 | 16.188 | 1.00 | 69.57 |
| 446 | CA | PHE C | 60 | 16.515 | 36.265 | 16.616 | 1.00 | 69.57 |
| 447 | CB | PHE C | 60 | 17.455 | 36.314 | 15.438 | 1.00 | 112.86 |
| 448 | CG | PHE C | 60 | 17.775 | 34.974 | 14.896 | 1.00 | 112.86 |
| 449 | CD1 | PHE C | 60 | 18.097 | 34.805 | 13.562 | 1.00 | 112.86 |
| 450 | CD2 | PHE C | 60 | 17.757 | 33.867 | 15.718 | 1.00 | 112.86 |
| 451 | CE1 | PHE C | 60 | 18.396 | 33.553 | 13.046 | 1.00 | 112.86 |
| 452 | CE2 | PHE C | 60 | 18.050 | 32.608 | 15.217 | 1.00 | 112.86 |
| 453 | CZ | PHE C | 60 | 18.372 | 32.452 | 13.877 | 1.00 | 112.86 |
| 454 | C | PHE C | 60 | 16.436 | 37.644 | 17.258 | 1.00 | 69.57 |
| 455 | O | PHE C | 60 | 17.213 | 37.958 | 18.172 | 1.00 | 69.57 |
| 456 | N | GLU C | 61 | 15.498 | 38.466 | 16.785 | 1.00 | 114.60 |
| 457 | CA | GLU C | 61 | 15.308 | 39.823 | 17.303 | 1.00 | 114.60 |
| 458 | CB | GLU C | 61 | 14.268 | 40.583 | 16.482 | 1.00 | 179.88 |
| 459 | CG | GLU C | 61 | 14.629 | 40.775 | 15.025 | 1.00 | 179.88 |
| 460 | CD | GLU C | 61 | 14.804 | 39.464 | 14.296 | 1.00 | 179.88 |
| 461 | OE1 | GLU C | 61 | 13.874 | 38.631 | 14.334 | 1.00 | 179.88 |
| 462 | OE2 | GLU C | 61 | 15.871 | 39.269 | 13.682 | 1.00 | 179.88 |
| 463 | C | GLU C | 61 | 14.865 | 39.831 | 18.757 | 1.00 | 114.60 |
| 464 | O | GLU C | 61 | 15.064 | 40.828 | 19.451 | 1.00 | 114.60 |
| 465 | N | ASP C | 62 | 14.251 | 38.737 | 19.214 | 1.00 | 61.26 |
| 466 | CA | ASP C | 62 | 13.807 | 38.654 | 20.605 | 1.00 | 61.26 |
| 467 | CB | ASP C | 62 | 12.884 | 37.457 | 20.801 | 1.00 | 109.78 |
| 468 | CG | ASP C | 62 | 11.707 | 37.472 | 19.842 | 1.00 | 109.78 |
| 469 | OD1 | ASP C | 62 | 11.182 | 38.574 | 19.564 | 1.00 | 109.78 |
| 470 | OD2 | ASP C | 62 | 11.296 | 36.385 | 19.374 | 1.00 | 109.78 |
| 471 | C | ASP C | 62 | 15.018 | 38.559 | 21.542 | 1.00 | 61.26 |
| 472 | O | ASP C | 62 | 14.915 | 38.859 | 22.726 | 1.00 | 61.26 |
| 473 | N | SER C | 63 | 16.166 | 38.159 | 20.999 | 1.00 | 49.60 |
| 474 | CA | SER C | 63 | 17.390 | 38.050 | 21.776 | 1.00 | 49.60 |
| 475 | CB | SER C | 63 | 18.539 | 37.599 | 20.873 | 1.00 | 59.31 |
| 476 | OG | SER C | 63 | 18.360 | 36.265 | 20.405 | 1.00 | 59.31 |
| 477 | C | SER C | 63 | 17.669 | 39.432 | 22.294 | 1.00 | 49.60 |
| 478 | O | SER C | 63 | 17.647 | 40.359 | 21.520 | 1.00 | 49.60 |
| 479 | N | GLY C | 64 | 17.918 | 39.595 | 23.583 | 1.00 | 66.63 |
| 480 | CA | GLY C | 64 | 18.192 | 40.936 | 24.070 | 1.00 | 66.63 |
| 481 | C | GLY C | 64 | 18.223 | 41.119 | 25.579 | 1.00 | 66.63 |
| 482 | O | GLY C | 64 | 18.210 | 40.154 | 26.339 | 1.00 | 66.63 |
| 483 | N | GLU C | 65 | 18.238 | 42.371 | 26.018 | 1.00 | 55.08 |
| 484 | CA | GLU C | 65 | 18.306 | 42.725 | 27.440 | 1.00 | 55.08 |
| 485 | CB | GLU C | 65 | 19.339 | 43.828 | 27.632 | 1.00 | 156.42 |
| 486 | CG | GLU C | 65 | 19.349 | 44.480 | 28.979 | 1.00 | 156.42 |
| 487 | CD | GLU C | 65 | 20.163 | 45.756 | 28.973 | 1.00 | 156.42 |
| 488 | OE1 | GLU C | 65 | 19.785 | 46.700 | 28.254 | 1.00 | 156.42 |
| 489 | OE2 | GLU C | 65 | 21.183 | 45.817 | 29.696 | 1.00 | 156.42 |
| 490 | C | GLU C | 65 | 16.899 | 43.218 | 27.844 | 1.00 | 55.08 |
| 491 | O | GLU C | 65 | 16.346 | 44.123 | 27.213 | 1.00 | 55.08 |
| 492 | N | TYR C | 66 | 16.307 | 42.625 | 28.871 | 1.00 | 61.99 |
| 493 | CA | TYR C | 66 | 14.981 | 43.056 | 29.291 | 1.00 | 61.99 |
| 494 | CB | TYR C | 66 | 14.013 | 41.901 | 29.181 | 1.00 | 58.17 |
| 495 | CG | TYR C | 66 | 13.740 | 41.415 | 27.806 | 1.00 | 58.17 |
| 496 | CD1 | TYR C | 66 | 14.658 | 40.634 | 27.133 | 1.00 | 58.17 |
| 497 | CE1 | TYR C | 66 | 14.365 | 40.112 | 25.886 | 1.00 | 58.17 |
| 498 | CD2 | TYR C | 66 | 12.520 | 41.681 | 27.198 | 1.00 | 58.17 |
| 499 | CE2 | TYR C | 66 | 12.213 | 41.170 | 25.953 | 1.00 | 58.17 |
| 500 | CZ | TYR C | 66 | 13.134 | 40.379 | 25.300 | 1.00 | 58.17 |
| 501 | OH | TYR C | 66 | 12.786 | 39.826 | 24.081 | 1.00 | 58.17 |
| 502 | C | TYR C | 66 | 14.950 | 43.525 | 30.746 | 1.00 | 61.99 |
| 503 | O | TYR C | 66 | 15.850 | 43.192 | 31.522 | 1.00 | 61.99 |
| 504 | N | LYS C | 67 | 13.899 | 44.254 | 31.132 | 1.00 | 84.17 |
| 505 | CA | LYS C | 67 | 13.751 | 44.703 | 32.516 | 1.00 | 84.17 |
| 506 | CB | LYS C | 67 | 14.789 | 45.766 | 32.837 | 1.00 | 116.03 |
| 507 | CG | LYS C | 67 | 14.858 | 46.850 | 31.807 | 1.00 | 116.03 |
| 508 | CD | LYS C | 67 | 15.986 | 47.803 | 32.118 | 1.00 | 116.03 |
| 509 | CE | LYS C | 67 | 16.177 | 48.787 | 30.983 | 1.00 | 116.03 |
| 510 | NZ | LYS C | 67 | 17.324 | 49.693 | 31.252 | 1.00 | 116.03 |
| 511 | C | LYS C | 67 | 12.369 | 45.249 | 32.762 | 1.00 | 84.17 |
| 512 | O | LYS C | 67 | 11.696 | 45.655 | 31.819 | 1.00 | 84.17 |
| 513 | N | CYS C | 68 | 11.933 | 45.229 | 34.020 | 1.00 | 81.35 |
| 514 | CA | CYS C | 68 | 10.624 | 45.780 | 34.350 | 1.00 | 81.35 |
| 515 | C | CYS C | 68 | 10.749 | 46.788 | 35.467 | 1.00 | 81.35 |
| 516 | O | CYS C | 68 | 11.761 | 46.811 | 36.145 | 1.00 | 81.35 |
| 517 | CB | CYS C | 68 | 9.619 | 44.672 | 34.717 | 1.00 | 117.98 |
| 518 | SG | CYS C | 68 | 9.997 | 43.610 | 36.128 | 1.00 | 117.98 |
| 519 | N | GLN C | 69 | 9.734 | 47.628 | 35.627 | 1.00 | 106.08 |
| 520 | CA | GLN C | 69 | 9.722 | 48.638 | 36.664 | 1.00 | 106.08 |
| 521 | CB | GLN C | 69 | 10.471 | 49.900 | 36.188 | 1.00 | 124.18 |
| 522 | CG | GLN C | 69 | 10.166 | 51.173 | 36.978 | 1.00 | 124.18 |
| 523 | CD | GLN C | 69 | 10.841 | 52.407 | 36.397 | 1.00 | 124.18 |
| 524 | OE1 | GLN C | 69 | 10.720 | 52.687 | 35.205 | 1.00 | 124.18 |
| 525 | NE2 | GLN C | 69 | 11.542 | 53.160 | 37.244 | 1.00 | 124.18 |
| 526 | C | GLN C | 69 | 8.265 | 48.974 | 36.930 | 1.00 | 106.08 |
| 527 | O | GLN C | 69 | 7.416 | 48.787 | 36.054 | 1.00 | 106.08 |
| 528 | N | HIS C | 70 | 7.967 | 49.457 | 38.131 | 1.00 | 181.43 |
| 529 | CA | HIS C | 70 | 6.609 | 49.830 | 38.469 | 1.00 | 181.43 |
| 530 | CB | HIS C | 70 | 6.177 | 49.107 | 39.764 | 1.00 | 144.62 |
| 531 | CG | HIS C | 70 | 6.062 | 47.635 | 39.606 | 1.00 | 144.62 |
| 532 | CD2 | HIS C | 70 | 6.901 | 46.634 | 39.977 | 1.00 | 144.62 |
| 533 | ND1 | HIS C | 70 | 4.972 | 47.041 | 39.030 | 1.00 | 144.62 |
| 534 | CE1 | HIS C | 70 | 5.121 | 45.722 | 39.060 | 1.00 | 144.62 |
| 535 | NE2 | HIS C | 70 | 6.283 | 45.464 | 39.631 | 1.00 | 144.62 |
| 536 | C | HIS C | 70 | 6.421 | 51.335 | 38.609 | 1.00 | 181.43 |
| 537 | O | HIS C | 70 | 7.299 | 52.112 | 38.268 | 1.00 | 181.43 |
| 538 | N | GLN C | 71 | 5.254 | 51.714 | 39.108 | 1.00 | 249.25 |
| 539 | CA | GLN C | 71 | 4.925 | 53.108 | 39.290 | 1.00 | 249.25 |
| 540 | CB | GLN C | 71 | 3.550 | 53.209 | 39.950 | 1.00 | 249.45 |
| 541 | CG | GLN C | 71 | 2.717 | 54.409 | 39.544 | 1.00 | 249.45 |
| 542 | CD | GLN C | 71 | 2.659 | 54.624 | 38.057 | 1.00 | 249.45 |
| 543 | OE1 | GLN C | 71 | 1.970 | 53.886 | 37.367 | 1.00 | 249.45 |
| 544 | NE2 | GLN C | 71 | 3.360 | 55.626 | 37.553 | 1.00 | 249.45 |
| 545 | C | GLN C | 71 | 5.988 | 53.876 | 40.093 | 1.00 | 249.25 |
| 546 | O | GLN C | 71 | 6.510 | 54.891 | 39.634 | 1.00 | 249.25 |
| 547 | N | GLN C | 72 | 6.321 | 53.366 | 41.276 | 1.00 | 190.92 |
| 548 | CA | GLN C | 72 | 7.312 | 54.001 | 42.145 | 1.00 | 190.92 |
| 549 | CB | GLN C | 72 | 6.639 | 54.552 | 43.406 | 1.00 | 249.44 |
| 550 | CG | GLN C | 72 | 7.556 | 55.342 | 44.333 | 1.00 | 249.44 |
| 551 | CD | GLN C | 72 | 6.833 | 55.900 | 45.543 | 1.00 | 249.44 |
| 552 | OE1 | GLN C | 72 | 5.871 | 56.656 | 45.411 | 1.00 | 249.44 |
| 553 | NE2 | GLN C | 72 | 7.296 | 55.530 | 46.734 | 1.00 | 249.44 |
| 554 | C | GLN C | 72 | 8.427 | 53.034 | 42.546 | 1.00 | 190.92 |
| 555 | O | GLN C | 72 | 8.660 | 52.788 | 43.734 | 1.00 | 190.92 |
| 556 | N | VAL C | 73 | 9.118 | 52.481 | 41.556 | 1.00 | 211.52 |
| 557 | CA | VAL C | 73 | 10.183 | 51.531 | 41.836 | 1.00 | 211.52 |
| 558 | CB | VAL C | 73 | 9.649 | 50.092 | 41.803 | 1.00 | 215.95 |
| 559 | CG1 | VAL C | 73 | 10.667 | 49.148 | 42.403 | 1.00 | 215.95 |
| 560 | CG2 | VAL C | 73 | 8.338 | 50.011 | 42.539 | 1.00 | 215.95 |
| 561 | C | VAL C | 73 | 11.297 | 51.639 | 40.816 | 1.00 | 211.52 |
| 562 | O | VAL C | 73 | 11.053 | 51.942 | 39.649 | 1.00 | 211.52 |
| 563 | N | ASN C | 74 | 12.525 | 51.391 | 41.256 | 1.00 | 137.61 |
| 564 | CA | ASN C | 74 | 13.656 | 51.454 | 40.349 | 1.00 | 137.61 |
| 565 | CB | ASN C | 74 | 14.950 | 51.650 | 41.136 | 1.00 | 154.43 |
| 566 | CG | ASN C | 74 | 14.895 | 52.860 | 42.037 | 1.00 | 154.43 |
| 567 | OD1 | ASN C | 74 | 14.408 | 53.920 | 41.631 | 1.00 | 154.43 |
| 568 | ND2 | ASN C | 74 | 15.401 | 52.711 | 43.259 | 1.00 | 154.43 |
| 569 | C | ASN C | 74 | 13.708 | 50.169 | 39.523 | 1.00 | 137.61 |
| 570 | O | ASN C | 74 | 13.641 | 49.062 | 40.063 | 1.00 | 137.61 |
| 571 | N | GLU C | 75 | 13.807 | 50.341 | 38.205 | 1.00 | 102.99 |
| 572 | CA | GLU C | 75 | 13.862 | 49.236 | 37.248 | 1.00 | 102.99 |
| 573 | CB | GLU C | 75 | 14.305 | 49.764 | 35.881 | 1.00 | 231.35 |
| 574 | CG | GLU C | 75 | 15.294 | 50.916 | 35.952 | 1.00 | 231.35 |
| 575 | CD | GLU C | 75 | 15.534 | 51.559 | 34.601 | 1.00 | 231.35 |
| 576 | OE1 | GLU C | 75 | 14.547 | 51.970 | 33.952 | 1.00 | 231.35 |
| 577 | OE2 | GLU C | 75 | 16.710 | 51.657 | 34.190 | 1.00 | 231.35 |
| 578 | C | GLU C | 75 | 14.749 | 48.080 | 37.693 | 1.00 | 102.99 |
| 579 | O | GLU C | 75 | 15.794 | 48.281 | 38.296 | 1.00 | 102.99 |
| 580 | N | SER C | 76 | 14.305 | 46.868 | 37.383 | 1.00 | 85.19 |
| 581 | CA | SER C | 76 | 14.987 | 45.629 | 37.747 | 1.00 | 85.19 |
| 582 | CB | SER C | 76 | 14.101 | 44.446 | 37.402 | 1.00 | 104.06 |
| 583 | OG | SER C | 76 | 13.920 | 44.371 | 35.992 | 1.00 | 104.06 |
| 584 | C | SER C | 76 | 16.308 | 45.424 | 37.044 | 1.00 | 85.19 |
| 585 | O | SER C | 76 | 16.560 | 46.020 | 35.998 | 1.00 | 85.19 |
| 586 | N | GLU C | 77 | 17.140 | 44.553 | 37.612 | 1.00 | 76.77 |
| 587 | CA | GLU C | 77 | 18.444 | 44.238 | 37.023 | 1.00 | 76.77 |
| 588 | CB | GLU C | 77 | 19.263 | 43.355 | 37.962 | 1.00 | 228.57 |
| 589 | CG | GLU C | 77 | 19.643 | 44.039 | 39.268 | 1.00 | 228.57 |
| 590 | CD | GLU C | 77 | 20.577 | 45.228 | 39.070 | 1.00 | 228.57 |
| 591 | OE1 | GLU C | 77 | 20.709 | 45.703 | 37.921 | 1.00 | 228.57 |
| 592 | OE2 | GLU C | 77 | 21.172 | 45.695 | 40.068 | 1.00 | 228.57 |
| 593 | C | GLU C | 77 | 18.178 | 43.498 | 35.728 | 1.00 | 76.77 |
| 594 | O | GLU C | 77 | 17.573 | 42.420 | 35.746 | 1.00 | 76.77 |
| 595 | N | PRO C | 78 | 18.600 | 44.080 | 34.585 | 1.00 | 81.92 |
| 596 | CD | PRO C | 78 | 19.176 | 45.434 | 34.463 | 1.00 | 75.82 |
| 597 | CA | PRO C | 78 | 18.417 | 43.503 | 33.255 | 1.00 | 81.92 |
| 598 | CB | PRO C | 78 | 19.357 | 44.328 | 32.394 | 1.00 | 75.82 |
| 599 | CG | PRO C | 78 | 19.130 | 45.681 | 32.940 | 1.00 | 75.82 |
| 600 | C | PRO C | 78 | 18.717 | 42.029 | 33.190 | 1.00 | 81.92 |
| 601 | O | PRO C | 78 | 19.475 | 41.508 | 34.000 | 1.00 | 81.92 |
| 602 | N | VAL C | 79 | 18.094 | 41.357 | 32.233 | 1.00 | 71.44 |
| 603 | CA | VAL C | 79 | 18.300 | 39.938 | 32.036 | 1.00 | 71.44 |
| 604 | CB | VAL C | 79 | 17.121 | 39.139 | 32.538 | 1.00 | 74.89 |
| 605 | CG1 | VAL C | 79 | 17.199 | 37.717 | 32.029 | 1.00 | 74.89 |
| 606 | CG2 | VAL C | 79 | 17.131 | 39.154 | 34.047 | 1.00 | 74.89 |
| 607 | C | VAL C | 79 | 18.454 | 39.707 | 30.552 | 1.00 | 71.44 |
| 608 | O | VAL C | 79 | 17.646 | 40.184 | 29.748 | 1.00 | 71.44 |
| 609 | N | TYR C | 80 | 19.502 | 38.989 | 30.179 | 1.00 | 69.00 |
| 610 | CA | TYR C | 80 | 19.710 | 38.728 | 28.773 | 1.00 | 69.00 |
| 611 | CB | TYR C | 80 | 21.184 | 38.747 | 28.406 | 1.00 | 132.22 |
| 612 | CG | TYR C | 80 | 21.361 | 38.905 | 26.921 | 1.00 | 132.22 |
| 613 | CD1 | TYR C | 80 | 20.962 | 40.075 | 26.284 | 1.00 | 132.22 |
| 614 | CE1 | TYR C | 80 | 21.102 | 40.236 | 24.923 | 1.00 | 132.22 |
| 615 | CD2 | TYR C | 80 | 21.902 | 37.891 | 26.143 | 1.00 | 132.22 |
| 616 | CE2 | TYR C | 80 | 22.039 | 38.046 | 24.754 | 1.00 | 132.22 |
| 617 | CZ | TYR C | 80 | 21.636 | 39.228 | 24.163 | 1.00 | 132.22 |
| 618 | OH | TYR C | 80 | 21.777 | 39.415 | 22.807 | 1.00 | 132.22 |
| 619 | C | TYR C | 80 | 19.150 | 37.401 | 28.300 | 1.00 | 69.00 |
| 620 | O | TYR C | 80 | 19.295 | 36.380 | 28.939 | 1.00 | 69.00 |
| 621 | N | LEU C | 81 | 18.534 | 37.423 | 27.141 | 1.00 | 61.97 |
| 622 | CA | LEU C | 81 | 17.988 | 36.232 | 26.557 | 1.00 | 61.97 |
| 623 | CB | LEU C | 81 | 16.501 | 36.449 | 26.351 | 1.00 | 56.21 |
| 624 | CG | LEU C | 81 | 15.878 | 35.304 | 25.589 | 1.00 | 56.21 |
| 625 | CD1 | LEU C | 81 | 16.017 | 34.060 | 26.431 | 1.00 | 56.21 |
| 626 | CD2 | LEU C | 81 | 14.424 | 35.608 | 25.294 | 1.00 | 56.21 |
| 627 | C | LEU C | 81 | 18.700 | 36.019 | 25.213 | 1.00 | 61.97 |
| 628 | O | LEU C | 81 | 18.814 | 36.949 | 24.423 | 1.00 | 61.97 |
| 629 | N | GLU C | 82 | 19.191 | 34.816 | 24.948 | 1.00 | 70.61 |
| 630 | CA | GLU C | 82 | 19.855 | 34.581 | 23.674 | 1.00 | 70.61 |
| 631 | CB | GLU C | 82 | 21.326 | 34.242 | 23.882 | 1.00 | 114.25 |
| 632 | CG | GLU C | 82 | 22.179 | 34.569 | 22.675 | 1.00 | 114.25 |
| 633 | CD | GLU C | 82 | 23.657 | 34.256 | 22.884 | 1.00 | 114.25 |
| 634 | OE1 | GLU C | 82 | 24.149 | 34.456 | 24.020 | 1.00 | 114.25 |
| 635 | OE2 | GLU C | 82 | 24.331 | 33.827 | 21.913 | 1.00 | 114.25 |
| 636 | C | GLU C | 82 | 19.172 | 33.457 | 22.896 | 1.00 | 70.61 |
| 637 | O | GLU C | 82 | 18.913 | 32.381 | 23.440 | 1.00 | 70.61 |
| 638 | N | VAL C | 83 | 18.886 | 33.706 | 21.622 | 1.00 | 70.46 |
| 639 | CA | VAL C | 83 | 18.225 | 32.717 | 20.795 | 1.00 | 70.46 |
| 640 | CB | VAL C | 83 | 17.114 | 33.346 | 20.004 | 1.00 | 68.26 |
| 641 | CG1 | VAL C | 83 | 16.531 | 32.318 | 19.057 | 1.00 | 68.26 |
| 642 | CG2 | VAL C | 83 | 16.045 | 33.848 | 20.941 | 1.00 | 68.26 |
| 643 | C | VAL C | 83 | 19.156 | 32.014 | 19.825 | 1.00 | 70.46 |
| 644 | O | VAL C | 83 | 19.955 | 32.663 | 19.157 | 1.00 | 70.46 |
| 645 | N | PHE C | 84 | 19.031 | 30.694 | 19.718 | 1.00 | 54.26 |
| 646 | CA | PHE C | 84 | 19.913 | 29.944 | 18.845 | 1.00 | 54.26 |
| 647 | CB | PHE C | 84 | 20.793 | 28.988 | 19.650 | 1.00 | 65.02 |
| 648 | CG | PHE C | 84 | 21.645 | 29.643 | 20.638 | 1.00 | 65.02 |
| 649 | CD1 | PHE C | 84 | 21.104 | 30.128 | 21.788 | 1.00 | 65.02 |
| 650 | CD2 | PHE C | 84 | 22.996 | 29.767 | 20.426 | 1.00 | 65.02 |
| 651 | CE1 | PHE C | 84 | 21.897 | 30.746 | 22.733 | 1.00 | 65.02 |
| 652 | CE2 | PHE C | 84 | 23.811 | 30.384 | 21.359 | 1.00 | 65.02 |
| 653 | CZ | PHE C | 84 | 23.262 | 30.872 | 22.519 | 1.00 | 65.02 |
| 654 | C | PHE C | 84 | 19.247 | 29.092 | 17.790 | 1.00 | 54.26 |
| 655 | O | PHE C | 84 | 18.045 | 28.781 | 17.861 | 1.00 | 54.26 |
| 656 | N | SER C | 85 | 20.101 | 28.686 | 16.849 | 1.00 | 85.79 |
| 657 | CA | SER C | 85 | 19.768 | 27.807 | 15.750 | 1.00 | 85.79 |
| 658 | CB | SER C | 85 | 19.683 | 28.583 | 14.435 | 1.00 | 134.11 |
| 659 | OG | SER C | 85 | 19.375 | 27.719 | 13.355 | 1.00 | 134.11 |
| 660 | C | SER C | 85 | 20.951 | 26.860 | 15.700 | 1.00 | 85.79 |
| 661 | O | SER C | 85 | 22.063 | 27.274 | 15.360 | 1.00 | 85.79 |
| 662 | N | ASP C | 86 | 20.731 | 25.607 | 16.083 | 1.00 | 52.64 |
| 663 | CA | ASP C | 86 | 21.798 | 24.604 | 16.049 | 1.00 | 52.64 |
| 664 | CB | ASP C | 86 | 22.912 | 24.984 | 17.015 | 1.00 | 115.87 |
| 665 | CG | ASP C | 86 | 24.265 | 24.581 | 16.504 | 1.00 | 115.87 |
| 666 | OD1 | ASP C | 86 | 24.448 | 23.384 | 16.194 | 1.00 | 115.87 |
| 667 | OD2 | ASP C | 86 | 25.144 | 25.459 | 16.411 | 1.00 | 115.87 |
| 668 | C | ASP C | 86 | 21.199 | 23.235 | 16.420 | 1.00 | 52.64 |
| 669 | O | ASP C | 86 | 20.051 | 23.168 | 16.879 | 1.00 | 52.64 |
| 670 | N | TRP C | 87 | 21.944 | 22.146 | 16.202 | 1.00 | 58.25 |
| 671 | CA | TRP C | 87 | 21.424 | 20.809 | 16.522 | 1.00 | 58.25 |
| 672 | CB | TRP C | 87 | 22.372 | 19.723 | 16.049 | 1.00 | 247.83 |
| 673 | CG | TRP C | 87 | 22.083 | 19.354 | 14.675 | 1.00 | 247.83 |
| 674 | CD2 | TRP C | 87 | 22.700 | 19.899 | 13.518 | 1.00 | 247.83 |
| 675 | CE2 | TRP C | 87 | 22.052 | 19.344 | 12.404 | 1.00 | 247.83 |
| 676 | CE3 | TRP C | 87 | 23.742 | 20.812 | 13.311 | 1.00 | 247.83 |
| 677 | CD1 | TRP C | 87 | 21.106 | 18.504 | 14.235 | 1.00 | 247.83 |
| 678 | NE1 | TRP C | 87 | 21.082 | 18.496 | 12.867 | 1.00 | 247.83 |
| 679 | CZ2 | TRP C | 87 | 22.418 | 19.660 | 11.113 | 1.00 | 247.83 |
| 680 | CZ3 | TRP C | 87 | 24.098 | 21.132 | 12.023 | 1.00 | 247.83 |
| 681 | CH2 | TRP C | 87 | 23.444 | 20.552 | 10.944 | 1.00 | 247.83 |
| 682 | C | TRP C | 87 | 21.220 | 20.656 | 18.009 | 1.00 | 58.25 |
| 683 | O | TRP C | 87 | 20.141 | 20.260 | 18.463 | 1.00 | 58.25 |
| 684 | N | LEU C | 88 | 22.264 | 20.985 | 18.760 | 1.00 | 79.45 |
| 685 | CA | LEU C | 88 | 22.230 | 20.887 | 20.197 | 1.00 | 79.45 |
| 686 | CB | LEU C | 88 | 23.225 | 19.845 | 20.659 | 1.00 | 57.97 |
| 687 | CG | LEU C | 88 | 22.896 | 18.431 | 20.245 | 1.00 | 57.97 |
| 688 | CD1 | LEU C | 88 | 23.876 | 17.490 | 20.880 | 1.00 | 57.97 |
| 689 | CD2 | LEU C | 88 | 21.495 | 18.114 | 20.693 | 1.00 | 57.97 |
| 690 | C | LEU C | 88 | 22.558 | 22.194 | 20.870 | 1.00 | 79.45 |
| 691 | O | LEU C | 88 | 23.432 | 22.940 | 20.419 | 1.00 | 79.45 |
| 692 | N | LEU C | 89 | 21.872 | 22.451 | 21.976 | 1.00 | 57.52 |
| 693 | CA | LEU C | 89 | 22.097 | 23.669 | 22.732 | 1.00 | 57.52 |
| 694 | CB | LEU C | 89 | 20.919 | 24.616 | 22.578 | 1.00 | 59.32 |
| 695 | CG | LEU C | 89 | 21.105 | 25.884 | 23.373 | 1.00 | 59.32 |
| 696 | CD1 | LEU C | 89 | 22.513 | 26.439 | 23.134 | 1.00 | 59.32 |
| 697 | CD2 | LEU C | 89 | 20.081 | 26.861 | 22.941 | 1.00 | 59.32 |
| 698 | C | LEU C | 89 | 22.246 | 23.316 | 24.184 | 1.00 | 57.52 |
| 699 | O | LEU C | 89 | 21.432 | 22.577 | 24.697 | 1.00 | 57.52 |
| 700 | N | LEU C | 90 | 23.287 | 23.815 | 24.846 | 1.00 | 64.90 |
| 701 | CA | LEU C | 90 | 23.472 | 23.521 | 26.267 | 1.00 | 64.90 |
| 702 | CB | LEU C | 90 | 24.948 | 23.512 | 26.638 | 1.00 | 46.55 |
| 703 | CG | LEU C | 90 | 25.227 | 23.312 | 28.119 | 1.00 | 46.55 |
| 704 | CD1 | LEU C | 90 | 24.701 | 21.955 | 28.491 | 1.00 | 46.55 |
| 705 | CD2 | LEU C | 90 | 26.712 | 23.409 | 28.423 | 1.00 | 46.55 |
| 706 | C | LEU C | 90 | 22.781 | 24.591 | 27.083 | 1.00 | 64.90 |
| 707 | O | LEU C | 90 | 23.167 | 25.754 | 27.032 | 1.00 | 64.90 |
| 708 | N | GLN C | 91 | 21.769 | 24.208 | 27.852 | 1.00 | 56.08 |
| 709 | CA | GLN C | 91 | 21.052 | 25.187 | 28.650 | 1.00 | 56.08 |
| 710 | CB | GLN C | 91 | 19.573 | 24.944 | 28.517 | 1.00 | 49.57 |
| 711 | CG | GLN C | 91 | 19.115 | 25.036 | 27.094 | 1.00 | 49.57 |
| 712 | CD | GLN C | 91 | 17.606 | 25.013 | 26.979 | 1.00 | 49.57 |
| 713 | OE1 | GLN C | 91 | 16.947 | 23.977 | 27.245 | 1.00 | 49.57 |
| 714 | NE2 | GLN C | 91 | 17.030 | 26.167 | 26.601 | 1.00 | 49.57 |
| 715 | C | GLN C | 91 | 21.440 | 25.146 | 30.103 | 1.00 | 56.08 |
| 716 | O | GLN C | 91 | 21.697 | 24.066 | 30.638 | 1.00 | 56.08 |
| 717 | N | ALA C | 92 | 21.494 | 26.309 | 30.752 | 1.00 | 47.15 |
| 718 | CA | ALA C | 92 | 21.852 | 26.335 | 32.166 | 1.00 | 47.15 |
| 719 | CB | ALA C | 92 | 23.160 | 26.962 | 32.345 | 1.00 | 42.48 |
| 720 | C | ALA C | 92 | 20.828 | 27.102 | 32.955 | 1.00 | 47.15 |
| 721 | O | ALA C | 92 | 20.300 | 28.102 | 32.474 | 1.00 | 47.15 |
| 722 | N | SER C | 93 | 20.510 | 26.620 | 34.151 | 1.00 | 67.10 |
| 723 | CA | SER C | 93 | 19.541 | 27.289 | 35.013 | 1.00 | 67.10 |
| 724 | CB | SER C | 93 | 19.475 | 26.625 | 36.392 | 1.00 | 100.79 |
| 725 | OG | SER C | 93 | 20.758 | 26.434 | 36.965 | 1.00 | 100.79 |
| 726 | C | SER C | 93 | 20.040 | 28.705 | 35.137 | 1.00 | 67.10 |
| 727 | O | SER C | 93 | 19.395 | 29.632 | 34.692 | 1.00 | 67.10 |
| 728 | N | ALA C | 94 | 21.220 | 28.868 | 35.704 | 1.00 | 57.34 |
| 729 | CA | ALA C | 94 | 21.818 | 30.179 | 35.875 | 1.00 | 57.34 |
| 730 | CB | ALA C | 94 | 21.716 | 30.609 | 37.326 | 1.00 | 92.07 |
| 731 | C | ALA C | 94 | 23.279 | 30.072 | 35.462 | 1.00 | 57.34 |
| 732 | O | ALA C | 94 | 23.912 | 29.048 | 35.694 | 1.00 | 57.34 |
| 733 | N | GLU C | 95 | 23.825 | 31.123 | 34.860 | 1.00 | 69.09 |
| 734 | CA | GLU C | 95 | 25.212 | 31.088 | 34.419 | 1.00 | 69.09 |
| 735 | CB | GLU C | 95 | 25.403 | 31.994 | 33.214 | 1.00 | 142.79 |
| 736 | CG | GLU C | 95 | 24.526 | 31.618 | 32.048 | 1.00 | 142.79 |
| 737 | CD | GLU C | 95 | 24.954 | 32.288 | 30.759 | 1.00 | 142.79 |
| 738 | OE1 | GLU C | 95 | 24.268 | 32.084 | 29.733 | 1.00 | 142.79 |
| 739 | OE2 | GLU C | 95 | 25.976 | 33.013 | 30.767 | 1.00 | 142.79 |
| 740 | C | GLU C | 95 | 26.232 | 31.459 | 35.494 | 1.00 | 69.09 |
| 741 | O | GLU C | 95 | 27.435 | 31.238 | 35.316 | 1.00 | 69.09 |
| 742 | N | VAL C | 96 | 25.765 | 32.041 | 36.598 | 1.00 | 86.41 |
| 743 | CA | VAL C | 96 | 26.640 | 32.419 | 37.713 | 1.00 | 86.41 |
| 744 | CB | VAL C | 96 | 26.922 | 33.903 | 37.702 | 1.00 | 74.25 |
| 745 | CG1 | VAL C | 96 | 28.119 | 34.207 | 38.587 | 1.00 | 74.25 |
| 746 | CG2 | VAL C | 96 | 27.176 | 34.357 | 36.277 | 1.00 | 74.25 |
| 747 | C | VAL C | 96 | 25.910 | 32.052 | 38.990 | 1.00 | 86.41 |
| 748 | O | VAL C | 96 | 24.733 | 32.348 | 39.135 | 1.00 | 86.41 |
| 749 | N | VAL C | 97 | 26.610 | 31.434 | 39.931 | 1.00 | 73.13 |
| 750 | CA | VAL C | 97 | 25.953 | 30.955 | 41.142 | 1.00 | 73.13 |
| 751 | CB | VAL C | 97 | 25.697 | 29.456 | 41.001 | 1.00 | 48.19 |
| 752 | CG1 | VAL C | 97 | 24.767 | 28.999 | 42.037 | 1.00 | 48.19 |
| 753 | CG2 | VAL C | 97 | 25.176 | 29.146 | 39.634 | 1.00 | 48.19 |
| 754 | C | VAL C | 97 | 26.715 | 31.125 | 42.448 | 1.00 | 73.13 |
| 755 | O | VAL C | 97 | 27.924 | 30.893 | 42.513 | 1.00 | 73.13 |
| 756 | N | MET C | 98 | 25.999 | 31.490 | 43.503 | 1.00 | 70.97 |
| 757 | CA | MET C | 98 | 26.612 | 31.622 | 44.828 | 1.00 | 70.97 |
| 758 | CB | MET C | 98 | 25.638 | 32.331 | 45.763 | 1.00 | 151.84 |
| 759 | CG | MET C | 98 | 25.295 | 33.728 | 45.318 | 1.00 | 151.84 |
| 760 | SD | MET C | 98 | 26.581 | 34.857 | 45.790 | 1.00 | 151.84 |
| 761 | CE | MET C | 98 | 26.247 | 34.962 | 47.553 | 1.00 | 151.84 |
| 762 | C | MET C | 98 | 26.930 | 30.228 | 45.390 | 1.00 | 70.97 |
| 763 | O | MET C | 98 | 26.094 | 29.335 | 45.348 | 1.00 | 70.97 |
| 764 | N | GLU C | 99 | 28.130 | 30.037 | 45.923 | 1.00 | 61.59 |
| 765 | CA | GLU C | 99 | 28.508 | 28.740 | 46.475 | 1.00 | 61.59 |
| 766 | CB | GLU C | 99 | 29.762 | 28.874 | 47.339 | 1.00 | 200.85 |
| 767 | CG | GLU C | 99 | 30.525 | 27.574 | 47.520 | 1.00 | 200.85 |
| 768 | CD | GLU C | 99 | 31.561 | 27.657 | 48.623 | 1.00 | 200.85 |
| 769 | OE1 | GLU C | 99 | 32.197 | 28.724 | 48.761 | 1.00 | 200.85 |
| 770 | OE2 | GLU C | 99 | 31.746 | 26.651 | 49.342 | 1.00 | 200.85 |
| 771 | C | GLU C | 99 | 27.354 | 28.221 | 47.325 | 1.00 | 61.59 |
| 772 | O | GLU C | 99 | 26.851 | 28.934 | 48.184 | 1.00 | 61.59 |
| 773 | N | GLY C | 100 | 26.901 | 27.000 | 47.076 | 1.00 | 69.94 |
| 774 | CA | GLY C | 100 | 25.819 | 26.456 | 47.876 | 1.00 | 69.94 |
| 775 | C | GLY C | 100 | 24.468 | 26.355 | 47.191 | 1.00 | 69.94 |
| 776 | O | GLY C | 100 | 23.600 | 25.591 | 47.657 | 1.00 | 69.94 |
| 777 | N | GLN C | 101 | 24.266 | 27.111 | 46.105 | 1.00 | 57.42 |
| 778 | CA | GLN C | 101 | 22.990 | 27.083 | 45.370 | 1.00 | 57.42 |
| 779 | CB | GLN C | 101 | 22.778 | 28.399 | 44.634 | 1.00 | 124.38 |
| 780 | CG | GLN C | 101 | 22.627 | 29.570 | 45.551 | 1.00 | 124.38 |
| 781 | CD | GLN C | 101 | 21.628 | 29.295 | 46.641 | 1.00 | 124.38 |
| 782 | OE1 | GLN C | 101 | 21.911 | 28.587 | 47.605 | 1.00 | 124.38 |
| 783 | NE2 | GLN C | 101 | 20.438 | 29.839 | 46.485 | 1.00 | 124.38 |
| 784 | C | GLN C | 101 | 22.854 | 25.918 | 44.369 | 1.00 | 57.42 |
| 785 | O | GLN C | 101 | 23.834 | 25.238 | 44.031 | 1.00 | 57.42 |
| 786 | N | PRO C | 102 | 21.627 | 25.663 | 43.893 | 1.00 | 52.71 |
| 787 | CD | PRO C | 102 | 20.356 | 26.358 | 44.165 | 1.00 | 80.58 |
| 788 | CA | PRO C | 102 | 21.438 | 24.573 | 42.942 | 1.00 | 52.71 |
| 789 | CB | PRO C | 102 | 19.957 | 24.318 | 43.044 | 1.00 | 80.58 |
| 790 | CG | PRO C | 102 | 19.422 | 25.709 | 43.171 | 1.00 | 80.58 |
| 791 | C | PRO C | 102 | 21.870 | 25.005 | 41.531 | 1.00 | 52.71 |
| 792 | O | PRO C | 102 | 21.853 | 26.203 | 41.189 | 1.00 | 52.71 |
| 793 | N | LEU C | 103 | 22.242 | 24.033 | 40.705 | 1.00 | 64.68 |
| 794 | CA | LEU C | 103 | 22.661 | 24.323 | 39.343 | 1.00 | 64.68 |
| 795 | CB | LEU C | 103 | 24.172 | 24.347 | 39.273 | 1.00 | 81.00 |
| 796 | CG | LEU C | 103 | 24.605 | 24.608 | 37.838 | 1.00 | 81.00 |
| 797 | CD1 | LEU C | 103 | 24.136 | 25.987 | 37.435 | 1.00 | 81.00 |
| 798 | CD2 | LEU C | 103 | 26.113 | 24.486 | 37.718 | 1.00 | 81.00 |
| 799 | C | LEU C | 103 | 22.147 | 23.244 | 38.403 | 1.00 | 64.68 |
| 800 | O | LEU C | 103 | 22.418 | 22.063 | 38.642 | 1.00 | 64.68 |
| 801 | N | PHE C | 104 | 21.409 | 23.616 | 37.355 | 1.00 | 64.85 |
| 802 | CA | PHE C | 104 | 20.923 | 22.599 | 36.423 | 1.00 | 64.85 |
| 803 | CB | PHE C | 104 | 19.392 | 22.530 | 36.418 | 1.00 | 111.94 |
| 804 | CG | PHE C | 104 | 18.787 | 22.248 | 37.758 | 1.00 | 111.94 |
| 805 | CD1 | PHE C | 104 | 18.694 | 23.250 | 38.710 | 1.00 | 111.94 |
| 806 | CD2 | PHE C | 104 | 18.300 | 20.980 | 38.068 | 1.00 | 111.94 |
| 807 | CE1 | PHE C | 104 | 18.128 | 23.000 | 39.962 | 1.00 | 111.94 |
| 808 | CE2 | PHE C | 104 | 17.731 | 20.717 | 39.320 | 1.00 | 111.94 |
| 809 | CZ | PHE C | 104 | 17.644 | 21.732 | 40.268 | 1.00 | 111.94 |
| 810 | C | PHE C | 104 | 21.410 | 22.829 | 34.993 | 1.00 | 64.85 |
| 811 | O | PHE C | 104 | 21.254 | 23.915 | 34.462 | 1.00 | 64.85 |
| 812 | N | LEU C | 105 | 22.009 | 21.817 | 34.371 | 1.00 | 49.42 |
| 813 | CA | LEU C | 105 | 22.469 | 21.933 | 32.983 | 1.00 | 49.42 |
| 814 | CB | LEU C | 105 | 23.928 | 21.502 | 32.848 | 1.00 | 35.01 |
| 815 | CG | LEU C | 105 | 24.870 | 22.289 | 33.757 | 1.00 | 35.01 |
| 816 | CD1 | LEU C | 105 | 26.346 | 21.971 | 33.451 | 1.00 | 35.01 |
| 817 | CD2 | LEU C | 105 | 24.579 | 23.735 | 33.535 | 1.00 | 35.01 |
| 818 | C | LEU C | 105 | 21.603 | 21.000 | 32.181 | 1.00 | 49.42 |
| 819 | O | LEU C | 105 | 21.225 | 19.954 | 32.679 | 1.00 | 49.42 |
| 820 | N | ARG C | 106 | 21.290 | 21.353 | 30.946 | 1.00 | 67.77 |
| 821 | CA | ARG C | 106 | 20.438 | 20.487 | 30.155 | 1.00 | 67.77 |
| 822 | CB | ARG C | 106 | 19.027 | 21.043 | 30.194 | 1.00 | 104.09 |
| 823 | CG | ARG C | 106 | 18.056 | 20.302 | 29.334 | 1.00 | 104.09 |
| 824 | CD | ARG C | 106 | 16.745 | 21.071 | 29.217 | 1.00 | 104.09 |
| 825 | NE | ARG C | 106 | 15.814 | 20.401 | 28.316 | 1.00 | 104.09 |
| 826 | CZ | ARG C | 106 | 14.812 | 21.002 | 27.694 | 1.00 | 104.09 |
| 827 | NH1 | ARG C | 106 | 14.616 | 22.292 | 27.879 | 1.00 | 104.09 |
| 828 | NH2 | ARG C | 106 | 14.023 | 20.314 | 26.879 | 1.00 | 104.09 |
| 829 | C | ARG C | 106 | 20.911 | 20.391 | 28.710 | 1.00 | 67.77 |
| 830 | O | ARG C | 106 | 21.063 | 21.431 | 28.053 | 1.00 | 67.77 |
| 831 | N | CYS C | 107 | 21.160 | 19.179 | 28.205 | 1.00 | 64.22 |
| 832 | CA | CYS C | 107 | 21.599 | 19.074 | 26.819 | 1.00 | 64.22 |
| 833 | C | CYS C | 107 | 20.320 | 19.044 | 26.063 | 1.00 | 64.22 |
| 834 | O | CYS C | 107 | 19.579 | 18.067 | 26.143 | 1.00 | 64.22 |
| 835 | CB | CYS C | 107 | 22.396 | 17.808 | 26.547 | 1.00 | 74.81 |
| 836 | SG | CYS C | 107 | 23.369 | 17.892 | 24.999 | 1.00 | 74.81 |
| 837 | N | HIS C | 108 | 20.054 | 20.132 | 25.351 | 1.00 | 62.02 |
| 838 | CA | HIS C | 108 | 18.815 | 20.288 | 24.593 | 1.00 | 62.02 |
| 839 | CB | HIS C | 108 | 18.257 | 21.669 | 24.859 | 1.00 | 73.41 |
| 840 | CG | HIS C | 108 | 16.893 | 21.884 | 24.302 | 1.00 | 73.41 |
| 841 | CD2 | HIS C | 108 | 16.409 | 22.839 | 23.479 | 1.00 | 73.41 |
| 842 | ND1 | HIS C | 108 | 15.821 | 21.093 | 24.648 | 1.00 | 73.41 |
| 843 | CE1 | HIS C | 108 | 14.731 | 21.558 | 24.067 | 1.00 | 73.41 |
| 844 | NE2 | HIS C | 108 | 15.061 | 22.618 | 23.353 | 1.00 | 73.41 |
| 845 | C | HIS C | 108 | 18.925 | 20.085 | 23.089 | 1.00 | 62.02 |
| 846 | O | HIS C | 108 | 19.750 | 20.724 | 22.412 | 1.00 | 62.02 |
| 847 | N | GLY C | 109 | 18.066 | 19.207 | 22.578 | 1.00 | 82.12 |
| 848 | CA | GLY C | 109 | 18.075 | 18.907 | 21.161 | 1.00 | 82.12 |
| 849 | C | GLY C | 109 | 17.196 | 19.859 | 20.396 | 1.00 | 82.12 |
| 850 | O | GLY C | 109 | 16.281 | 20.439 | 20.963 | 1.00 | 82.12 |
| 851 | N | TRP C | 110 | 17.473 | 20.020 | 19.107 | 1.00 | 66.53 |
| 852 | CA | TRP C | 110 | 16.691 | 20.916 | 18.282 | 1.00 | 66.53 |
| 853 | CB | TRP C | 110 | 17.327 | 21.057 | 16.911 | 1.00 | 113.55 |
| 854 | CG | TRP C | 110 | 16.487 | 21.825 | 15.969 | 1.00 | 113.55 |
| 855 | CD2 | TRP C | 110 | 16.565 | 23.226 | 15.701 | 1.00 | 113.55 |
| 856 | CE2 | TRP C | 110 | 15.552 | 23.532 | 14.769 | 1.00 | 113.55 |
| 857 | CE3 | TRP C | 110 | 17.401 | 24.260 | 16.157 | 1.00 | 113.55 |
| 858 | CD1 | TRP C | 110 | 15.460 | 21.348 | 15.216 | 1.00 | 113.55 |
| 859 | NE1 | TRP C | 110 | 14.892 | 22.364 | 14.490 | 1.00 | 113.55 |
| 860 | CZ2 | TRP C | 110 | 15.345 | 24.831 | 14.280 | 1.00 | 113.55 |
| 861 | CZ3 | TRP C | 110 | 17.193 | 25.561 | 15.668 | 1.00 | 113.55 |
| 862 | CH2 | TRP C | 110 | 16.171 | 25.829 | 14.742 | 1.00 | 113.55 |
| 863 | C | TRP C | 110 | 15.284 | 20.383 | 18.160 | 1.00 | 66.53 |
| 864 | O | TRP C | 110 | 15.060 | 19.188 | 18.276 | 1.00 | 66.53 |
| 865 | N | ARG C | 111 | 14.334 | 21.285 | 17.951 | 1.00 | 82.69 |
| 866 | CA | ARG C | 111 | 12.928 | 20.924 | 17.807 | 1.00 | 82.69 |
| 867 | CB | ARG C | 111 | 12.677 | 20.368 | 16.432 | 1.00 | 249.07 |
| 868 | CG | ARG C | 111 | 12.367 | 21.439 | 15.493 | 1.00 | 249.07 |
| 869 | CD | ARG C | 111 | 11.908 | 20.831 | 14.270 | 1.00 | 249.07 |
| 870 | NE | ARG C | 111 | 10.767 | 21.587 | 13.769 | 1.00 | 249.07 |
| 871 | CZ | ARG C | 111 | 9.568 | 21.660 | 14.334 | 1.00 | 249.07 |
| 872 | NH1 | ARG C | 111 | 9.309 | 20.994 | 15.438 | 1.00 | 249.07 |
| 873 | NH2 | ARG C | 111 | 8.601 | 22.346 | 13.736 | 1.00 | 249.07 |
| 874 | C | ARG C | 111 | 12.433 | 19.928 | 18.828 | 1.00 | 82.69 |
| 875 | O | ARG C | 111 | 11.471 | 19.213 | 18.595 | 1.00 | 82.69 |
| 876 | N | ASN C | 112 | 13.119 | 19.872 | 19.953 | 1.00 | 79.91 |
| 877 | CA | ASN C | 112 | 12.756 | 18.976 | 21.027 | 1.00 | 79.91 |
| 878 | CB | ASN C | 112 | 11.354 | 19.288 | 21.540 | 1.00 | 134.30 |
| 879 | CG | ASN C | 112 | 11.152 | 18.815 | 22.957 | 1.00 | 134.30 |
| 880 | OD1 | ASN C | 112 | 11.850 | 17.902 | 23.427 | 1.00 | 134.30 |
| 881 | ND2 | ASN C | 112 | 10.194 | 19.424 | 23.653 | 1.00 | 134.30 |
| 882 | C | ASN C | 112 | 12.833 | 17.513 | 20.651 | 1.00 | 79.91 |
| 883 | O | ASN C | 112 | 12.172 | 16.683 | 21.270 | 1.00 | 79.91 |
| 884 | N | TRP C | 113 | 13.637 | 17.180 | 19.650 | 1.00 | 91.68 |
| 885 | CA | TRP C | 113 | 13.771 | 15.780 | 19.287 | 1.00 | 91.68 |
| 886 | CB | TRP C | 113 | 14.648 | 15.601 | 18.062 | 1.00 | 105.58 |
| 887 | CG | TRP C | 113 | 13.958 | 15.923 | 16.805 | 1.00 | 105.58 |
| 888 | CD2 | TRP C | 113 | 14.528 | 16.557 | 15.662 | 1.00 | 105.58 |
| 889 | CE2 | TRP C | 113 | 13.524 | 16.609 | 14.670 | 1.00 | 105.58 |
| 890 | CE3 | TRP C | 113 | 15.803 | 17.083 | 15.373 | 1.00 | 105.58 |
| 891 | CD1 | TRP C | 113 | 12.660 | 15.629 | 16.478 | 1.00 | 105.58 |
| 892 | NE1 | TRP C | 113 | 12.393 | 16.038 | 15.194 | 1.00 | 105.58 |
| 893 | CZ2 | TRP C | 113 | 13.750 | 17.164 | 13.413 | 1.00 | 105.58 |
| 894 | CZ3 | TRP C | 113 | 16.030 | 17.637 | 14.119 | 1.00 | 105.58 |
| 895 | CH2 | TRP C | 113 | 15.005 | 17.676 | 13.155 | 1.00 | 105.58 |
| 896 | C | TRP C | 113 | 14.393 | 15.003 | 20.425 | 1.00 | 91.68 |
| 897 | O | TRP C | 113 | 14.528 | 15.502 | 21.543 | 1.00 | 91.68 |
| 898 | N | ASP C | 114 | 14.780 | 13.770 | 20.133 | 1.00 | 96.80 |
| 899 | CA | ASP C | 114 | 15.398 | 12.926 | 21.133 | 1.00 | 96.80 |
| 900 | CB | ASP C | 114 | 14.675 | 11.576 | 21.213 | 1.00 | 249.33 |
| 901 | CG | ASP C | 114 | 13.402 | 11.645 | 22.044 | 1.00 | 249.33 |
| 902 | OD1 | ASP C | 114 | 13.493 | 12.001 | 23.239 | 1.00 | 249.33 |
| 903 | OD2 | ASP C | 114 | 12.314 | 11.344 | 21.507 | 1.00 | 249.33 |
| 904 | C | ASP C | 114 | 16.866 | 12.727 | 20.813 | 1.00 | 96.80 |
| 905 | O | ASP C | 114 | 17.257 | 12.561 | 19.652 | 1.00 | 96.80 |
| 906 | N | VAL C | 115 | 17.678 | 12.771 | 21.860 | 1.00 | 71.72 |
| 907 | CA | VAL C | 115 | 19.112 | 12.593 | 21.728 | 1.00 | 71.72 |
| 908 | CB | VAL C | 115 | 19.875 | 13.783 | 22.274 | 1.00 | 77.93 |
| 909 | CG1 | VAL C | 115 | 21.344 | 13.665 | 21.884 | 1.00 | 77.93 |
| 910 | CG2 | VAL C | 115 | 19.257 | 15.061 | 21.751 | 1.00 | 77.93 |
| 911 | C | VAL C | 115 | 19.535 | 11.382 | 22.516 | 1.00 | 71.72 |
| 912 | O | VAL C | 115 | 18.999 | 11.111 | 23.589 | 1.00 | 71.72 |
| 913 | N | TYR C | 116 | 20.502 | 10.653 | 21.979 | 1.00 | 67.55 |
| 914 | CA | TYR C | 116 | 20.999 | 9.454 | 22.644 | 1.00 | 67.55 |
| 915 | CB | TYR C | 116 | 20.610 | 8.219 | 21.826 | 1.00 | 100.42 |
| 916 | CG | TYR C | 116 | 19.121 | 8.010 | 21.689 | 1.00 | 100.42 |
| 917 | CD1 | TYR C | 116 | 18.431 | 8.465 | 20.572 | 1.00 | 100.42 |
| 918 | CE1 | TYR C | 116 | 17.049 | 8.284 | 20.454 | 1.00 | 100.42 |
| 919 | CD2 | TYR C | 116 | 18.403 | 7.371 | 22.690 | 1.00 | 100.42 |
| 920 | CE2 | TYR C | 116 | 17.028 | 7.185 | 22.590 | 1.00 | 100.42 |
| 921 | CZ | TYR C | 116 | 16.352 | 7.644 | 21.468 | 1.00 | 100.42 |
| 922 | OH | TYR C | 116 | 14.991 | 7.468 | 21.357 | 1.00 | 100.42 |
| 923 | C | TYR C | 116 | 22.514 | 9.501 | 22.853 | 1.00 | 67.55 |
| 924 | O | TYR C | 116 | 23.187 | 10.404 | 22.351 | 1.00 | 67.55 |
| 925 | N | LYS C | 117 | 23.040 | 8.518 | 23.578 | 1.00 | 124.33 |
| 926 | CA | LYS C | 117 | 24.466 | 8.443 | 23.848 | 1.00 | 124.33 |
| 927 | CB | LYS C | 117 | 25.233 | 7.946 | 22.617 | 1.00 | 168.92 |
| 928 | CG | LYS C | 117 | 25.319 | 6.439 | 22.465 | 1.00 | 168.92 |
| 929 | CD | LYS C | 117 | 26.431 | 6.073 | 21.494 | 1.00 | 168.92 |
| 930 | CE | LYS C | 117 | 27.782 | 6.589 | 21.988 | 1.00 | 168.92 |
| 931 | NZ | LYS C | 117 | 28.904 | 6.284 | 21.056 | 1.00 | 168.92 |
| 932 | C | LYS C | 117 | 24.974 | 9.822 | 24.229 | 1.00 | 124.33 |
| 933 | O | LYS C | 117 | 25.904 | 10.347 | 23.608 | 1.00 | 124.33 |
| 934 | N | VAL C | 118 | 24.361 | 10.406 | 25.253 | 1.00 | 96.88 |
| 935 | CA | VAL C | 118 | 24.752 | −11.731 | 25.714 | 1.00 | 96.88 |
| 936 | CB | VAL C | 118 | 23.572 | 12.432 | 26.338 | 1.00 | 47.91 |
| 937 | CG1 | VAL C | 118 | 24.036 | 13.463 | 27.307 | 1.00 | 47.91 |
| 938 | CG2 | VAL C | 118 | 22.786 | 13.114 | 25.264 | 1.00 | 47.91 |
| 939 | C | VAL C | 118 | 25.914 | 11.799 | 26.692 | 1.00 | 96.88 |
| 940 | O | VAL C | 118 | 25.980 | 11.030 | 27.648 | 1.00 | 96.88 |
| 941 | N | ILE C | 119 | 26.815 | 12.746 | 26.455 | 1.00 | 44.35 |
| 942 | CA | ILE C | 119 | 27.968 | 12.917 | 27.316 | 1.00 | 44.35 |
| 943 | CB | ILE C | 119 | 29.214 | 12.377 | 26.650 | 1.00 | 99.63 |
| 944 | CG2 | ILE C | 119 | 30.395 | 12.468 | 27.585 | 1.00 | 99.63 |
| 945 | CG1 | ILE C | 119 | 28.973 | 10.939 | 26.239 | 1.00 | 99.63 |
| 946 | CD1 | ILE C | 119 | 30.044 | 10.421 | 25.309 | 1.00 | 99.63 |
| 947 | C | ILE C | 119 | 28.227 | 14.396 | 27.618 | 1.00 | 44.35 |
| 948 | O | ILE C | 119 | 28.466 | 15.166 | 26.683 | 1.00 | 44.35 |
| 949 | N | TYR C | 120 | 28.193 | 14.816 | 28.889 | 1.00 | 48.39 |
| 950 | CA | TYR C | 120 | 28.478 | 16.224 | 29.185 | 1.00 | 48.39 |
| 951 | CB | TYR C | 120 | 27.803 | 16.687 | 30.458 | 1.00 | 42.29 |
| 952 | CG | TYR C | 120 | 26.322 | 16.785 | 30.363 | 1.00 | 42.29 |
| 953 | CD1 | TYR C | 120 | 25.537 | 15.697 | 30.581 | 1.00 | 42.29 |
| 954 | CE1 | TYR C | 120 | 24.185 | 15.775 | 30.485 | 1.00 | 42.29 |
| 955 | CD2 | TYR C | 120 | 25.710 | 17.982 | 30.035 | 1.00 | 42.29 |
| 956 | CE2 | TYR C | 120 | 24.340 | 18.088 | 29.924 | 1.00 | 42.29 |
| 957 | CZ | TYR C | 120 | 23.584 | 16.968 | 30.155 | 1.00 | 42.29 |
| 958 | OH | TYR C | 120 | 22.206 | 17.007 | 30.061 | 1.00 | 42.29 |
| 959 | C | TYR C | 120 | 29.962 | 16.358 | 29.412 | 1.00 | 48.39 |
| 960 | O | TYR C | 120 | 30.602 | 15.447 | 29.935 | 1.00 | 48.39 |
| 961 | N | TYR C | 121 | 30.518 | 17.498 | 29.053 | 1.00 | 53.29 |
| 962 | CA | TYR C | 121 | 31.942 | 17.700 | 29.246 | 1.00 | 53.29 |
| 963 | CB | TYR C | 121 | 32.664 | 17.828 | 27.887 | 1.00 | 75.15 |
| 964 | CG | TYR C | 121 | 32.747 | 16.569 | 27.044 | 1.00 | 75.15 |
| 965 | CD1 | TYR C | 121 | 31.598 | 15.971 | 26.525 | 1.00 | 75.15 |
| 966 | CE1 | TYR C | 121 | 31.669 | 14.849 | 25.717 | 1.00 | 75.15 |
| 967 | CD2 | TYR C | 121 | 33.976 | 16.002 | 26.731 | 1.00 | 75.15 |
| 968 | CE2 | TYR C | 121 | 34.055 | 14.881 | 25.921 | 1.00 | 75.15 |
| 969 | CZ | TYR C | 121 | 32.895 | 14.315 | 25.421 | 1.00 | 75.15 |
| 970 | OH | TYR C | 121 | 32.960 | 13.214 | 24.613 | 1.00 | 75.15 |
| 971 | C | TYR C | 121 | 32.219 | 18.966 | 30.056 | 1.00 | 53.29 |
| 972 | O | TYR C | 121 | 31.547 | 19.984 | 29.872 | 1.00 | 53.29 |
| 973 | N | LYS C | 122 | 33.199 | 18.908 | 30.954 | 1.00 | 72.38 |
| 974 | CA | LYS C | 122 | 33.580 | 20.077 | 31.725 | 1.00 | 72.38 |
| 975 | CB | LYS C | 122 | 33.217 | 19.930 | 33.197 | 1.00 | 98.98 |
| 976 | CG | LYS C | 122 | 33.582 | 21.162 | 34.003 | 1.00 | 98.98 |
| 977 | CD | LYS C | 122 | 33.532 | 20.901 | 35.481 | 1.00 | 98.98 |
| 978 | CE | LYS C | 122 | 34.071 | 22.074 | 36.260 | 1.00 | 98.98 |
| 979 | NZ | LYS C | 122 | 34.151 | 21.706 | 37.694 | 1.00 | 98.98 |
| 980 | C | LYS C | 122 | 35.080 | 20.238 | 31.595 | 1.00 | 72.38 |
| 981 | O | LYS C | 122 | 35.836 | 19.379 | 32.017 | 1.00 | 72.38 |
| 982 | N | ASP C | 123 | 35.507 | 21.342 | 31.001 | 1.00 | 92.01 |
| 983 | CA | ASP C | 123 | 36.918 | 21.628 | 30.809 | 1.00 | 92.01 |
| 984 | CB | ASP C | 123 | 37.606 | 21.819 | 32.161 | 1.00 | 107.76 |
| 985 | CG | ASP C | 123 | 37.288 | 23.167 | 32.785 | 1.00 | 107.76 |
| 986 | OD1 | ASP C | 123 | 37.362 | 24.186 | 32.052 | 1.00 | 107.76 |
| 987 | OD2 | ASP C | 123 | 36.979 | 23.215 | 34.000 | 1.00 | 107.76 |
| 983 | C | ASP C | 123 | 37.613 | 20.553 | 29.996 | 1.00 | 92.01 |
| 989 | O | ASP C | 123 | 38.719 | 20.144 | 30.322 | 1.00 | 92.01 |
| 990 | N | GLY C | 124 | 36.956 | 20.110 | 28.929 | 1.00 | 87.40 |
| 991 | CA | GLY C | 124 | 37.516 | 19.094 | 28.054 | 1.00 | 87.40 |
| 992 | C | GLY C | 124 | 37.465 | 17.665 | 28.570 | 1.00 | 87.40 |
| 993 | O | GLY C | 124 | 37.795 | 16.741 | 27.826 | 1.00 | 87.40 |
| 994 | N | GLU C | 125 | 37.047 | 17.474 | 29.821 | 1.00 | 63.88 |
| 995 | CA | GLU C | 125 | 36.991 | 16.139 | 30.427 | 1.00 | 63.88 |
| 996 | CB | GLU C | 125 | 37.331 | 16.197 | 31.931 | 1.00 | 184.13 |
| 997 | CG | GLU C | 125 | 38.775 | 16.547 | 32.294 | 1.00 | 184.13 |
| 998 | CD | GLU C | 125 | 39.723 | 15.367 | 32.176 | 1.00 | 184.13 |
| 999 | OE1 | GLU C | 125 | 39.524 | 14.369 | 32.903 | 1.00 | 184.13 |
| 1000 | OE2 | GLU C | 125 | 40.665 | 15.445 | 31.359 | 1.00 | 184.13 |
| 1001 | C | GLU C | 125 | 35.626 | 15.490 | 30.284 | 1.00 | 63.88 |
| 1002 | O | GLU C | 125 | 34.611 | 16.164 | 30.370 | 1.00 | 63.88 |
| 1003 | N | ALA C | 126 | 35.587 | 14.182 | 30.067 | 1.00 | 91.37 |
| 1004 | CA | ALA C | 126 | 34.302 | 13.501 | 29.985 | 1.00 | 91.37 |
| 1005 | CB | ALA C | 126 | 34.516 | 12.040 | 29.654 | 1.00 | 171.72 |
| 1006 | C | ALA C | 126 | 33.727 | 13.657 | 31.399 | 1.00 | 91.37 |
| 1007 | O | ALA C | 126 | 34.492 | 13.626 | 32.369 | 1.00 | 91.37 |
| 1008 | N | LEU C | 127 | 32.410 | 13.835 | 31.533 | 1.00 | 55.93 |
| 1009 | CA | LEU C | 127 | 31.815 | 14.011 | 32.861 | 1.00 | 55.93 |
| 1010 | CB | LEU C | 127 | 31.291 | 15.421 | 33.033 | 1.00 | 79.78 |
| 1011 | CG | LEU C | 127 | 31.277 | 15.727 | 34.519 | 1.00 | 79.78 |
| 1012 | CD1 | LEU C | 127 | 32.708 | 15.528 | 35.062 | 1.00 | 79.78 |
| 1013 | CD2 | LEU C | 127 | 30.796 | 17.133 | 34.754 | 1.00 | 79.78 |
| 1014 | C | LEU C | 127 | 30.722 | 13.050 | 33.267 | 1.00 | 55.93 |
| 1015 | O | LEU C | 127 | 30.851 | 12.396 | 34.292 | 1.00 | 55.93 |
| 1016 | N | LYS C | 128 | 29.633 | 12.998 | 32.501 | 1.00 | 71.66 |
| 1017 | CA | LYS C | 128 | 28.530 | 12.063 | 32.771 | 1.00 | 71.66 |
| 1018 | CB | LYS C | 128 | 27.354 | 12.774 | 33.419 | 1.00 | 111.82 |
| 1019 | CG | LYS C | 128 | 27.672 | 13.421 | 34.740 | 1.00 | 111.82 |
| 1020 | CD | LYS C | 128 | 27.814 | 12.412 | 35.853 | 1.00 | 111.82 |
| 1021 | CE | LYS C | 128 | 27.997 | 13.133 | 37.204 | 1.00 | 111.82 |
| 1022 | NZ | LYS C | 128 | 28.021 | 12.205 | 38.387 | 1.00 | 111.82 |
| 1023 | C | LYS C | 128 | 28.096 | 11.462 | 31.430 | 1.00 | 71.66 |
| 1024 | O | LYS C | 128 | 28.281 | 12.090 | 30.386 | 1.00 | 71.66 |
| 1025 | N | TYR C | 129 | 27.537 | 10.252 | 31.447 | 1.00 | 51.68 |
| 1026 | CA | TYR C | 129 | 27.110 | 9.616 | 30.208 | 1.00 | 51.68 |
| 1027 | CB | TYR C | 129 | 28.197 | 8.680 | 29.692 | 1.00 | 75.51 |
| 1028 | CG | TYR C | 129 | 27.655 | 7.647 | 28.732 | 1.00 | 75.51 |
| 1029 | CD1 | TYR C | 129 | 27.412 | 7.957 | 27.399 | 1.00 | 75.51 |
| 1030 | CE1 | TYR C | 129 | 26.846 | 7.041 | 26.529 | 1.00 | 75.51 |
| 1031 | CD2 | TYR C | 129 | 27.316 | 6.383 | 29.173 | 1.00 | 75.51 |
| 1032 | CE2 | TYR C | 129 | 26.739 | 5.454 | 28.309 | 1.00 | 75.51 |
| 1033 | CZ | TYR C | 129 | 26.510 | 5.789 | 26.992 | 1.00 | 75.51 |
| 1034 | OH | TYR C | 129 | 25.950 | 4.855 | 26.144 | 1.00 | 75.51 |
| 1035 | C | TYR C | 129 | 25.817 | 8.822 | 30.371 | 1.00 | 51.68 |
| 1036 | O | TYR C | 129 | 25.656 | 8.097 | 31.360 | 1.00 | 51.68 |
| 1037 | N | TRP C | 130 | 24.912 | 8.945 | 29.390 | 1.00 | 122.00 |
| 1038 | CA | TRP C | 130 | 23.641 | 8.226 | 29.404 | 1.00 | 122.00 |
| 1039 | CB | TRP C | 130 | 22.531 | 9.082 | 29.995 | 1.00 | 131.84 |
| 1040 | CG | TRP C | 130 | 22.854 | 9.740 | 31.299 | 1.00 | 131.84 |
| 1041 | CD2 | TRP C | 130 | 22.370 | 9.360 | 32.590 | 1.00 | 131.84 |
| 1042 | CE2 | TRP C | 130 | 22.886 | 10.292 | 33.518 | 1.00 | 131.84 |
| 1043 | CE3 | TRP C | 130 | 21.557 | 8.317 | 33.053 | 1.00 | 131.84 |
| 1044 | CD1 | TRP C | 130 | 23.613 | 10.850 | 31.489 | 1.00 | 131.84 |
| 1045 | NE1 | TRP C | 130 | 23.636 | 11.194 | 32.819 | 1.00 | 131.84 |
| 1046 | CZ2 | TRP C | 130 | 22.613 | 10.216 | 34.887 | 1.00 | 131.84 |
| 1047 | CZ3 | TRP C | 130 | 21.283 | 8.239 | 34.422 | 1.00 | 131.84 |
| 1048 | CH2 | TRP C | 130 | 21.809 | 9.190 | 35.319 | 1.00 | 131.84 |
| 1049 | C | TRP C | 130 | 23.199 | 7.805 | 28.010 | 1.00 | 122.00 |
| 1050 | O | TRP C | 130 | 23.720 | 8.292 | 27.015 | 1.00 | 122.00 |
| 1051 | N | TYR C | 131 | 22.229 | 6.898 | 27.944 | 1.00 | 94.11 |
| 1052 | CA | TYR C | 131 | 21.710 | 6.443 | 26.663 | 1.00 | 94.11 |
| 1053 | CB | TYR C | 131 | 21.108 | 5.048 | 26.756 | 1.00 | 199.39 |
| 1054 | CG | TYR C | 131 | 20.805 | 4.505 | 25.386 | 1.00 | 199.39 |
| 1055 | CD1 | TYR C | 131 | 21.836 | 4.074 | 24.552 | 1.00 | 199.39 |
| 1056 | CE1 | TYR C | 131 | 21.583 | 3.681 | 23.247 | 1.00 | 199.39 |
| 1057 | CD2 | TYR C | 131 | 19.503 | 4.522 | 24.879 | 1.00 | 199.39 |
| 1058 | CE2 | TYR C | 131 | 19.236 | 4.132 | 23.570 | 1.00 | 199.39 |
| 1059 | CZ | TYR C | 131 | 20.283 | 3.716 | 22.761 | 1.00 | 199.39 |
| 1060 | OH | TYR C | 131 | 20.033 | 3.369 | 21.456 | 1.00 | 199.39 |
| 1061 | C | TYR C | 131 | 20.629 | 7.440 | 26.306 | 1.00 | 94.11 |
| 1062 | O | TYR C | 131 | 20.894 | 8.379 | 25.554 | 1.00 | 94.11 |
| 1063 | N | GLU C | 132 | 19.411 | 7.221 | 26.817 | 1.00 | 108.28 |
| 1064 | CA | GLU C | 132 | 18.318 | 8.171 | 26.599 | 1.00 | 108.28 |
| 1065 | CB | GLU C | 132 | 16.986 | 7.670 | 27.185 | 1.00 | 249.42 |
| 1066 | CG | GLU C | 132 | 16.250 | 6.597 | 26.375 | 1.00 | 249.42 |
| 1067 | CD | GLU C | 132 | 14.874 | 7.058 | 25.901 | 1.00 | 249.42 |
| 1068 | OE1 | GLU C | 132 | 14.343 | 8.036 | 26.469 | 1.00 | 249.42 |
| 1069 | OE2 | GLU C | 132 | 14.320 | 6.434 | 24.968 | 1.00 | 249.42 |
| 1070 | C | GLU C | 132 | 18.899 | 9.243 | 27.491 | 1.00 | 108.28 |
| 1071 | O | GLU C | 132 | 19.243 | 8.953 | 28.636 | 1.00 | 108.28 |
| 1072 | N | ASN C | 133 | 19.029 | 10.468 | 26.989 | 1.00 | 132.29 |
| 1073 | CA | ASN C | 133 | 19.665 | 11.502 | 27.790 | 1.00 | 132.29 |
| 1074 | CB | ASN C | 133 | 19.946 | 12.758 | 26.960 | 1.00 | 123.07 |
| 1075 | CG | ASN C | 133 | 18.786 | 13.702 | 26.928 | 1.00 | 123.07 |
| 1076 | OD1 | ASN C | 133 | 17.659 | 13.297 | 26.651 | 1.00 | 123.07 |
| 1077 | ND2 | ASN C | 133 | 19.047 | 14.979 | 27.200 | 1.00 | 123.07 |
| 1078 | C | ASN C | 133 | 18.983 | 11.879 | 29.090 | 1.00 | 132.29 |
| 1079 | O | ASN C | 133 | 17.884 | 11.434 | 29.412 | 1.00 | 132.29 |
| 1080 | N | HIS C | 134 | 19.677 | 12.730 | 29.824 | 1.00 | 77.87 |
| 1081 | CA | HIS C | 134 | 19.271 | 13.173 | 31.140 | 1.00 | 77.87 |
| 1082 | CB | HIS C | 134 | 20.089 | 12.388 | 32.162 | 1.00 | 247.23 |
| 1083 | CG | HIS C | 134 | 19.688 | 12.628 | 33.579 | 1.00 | 247.23 |
| 1084 | CD2 | HIS C | 134 | 20.364 | 13.171 | 34.619 | 1.00 | 247.23 |
| 1085 | ND1 | HIS C | 134 | 18.451 | 12.270 | 34.071 | 1.00 | 247.23 |
| 1086 | CE1 | HIS C | 134 | 18.385 | 12.581 | 35.352 | 1.00 | 247.23 |
| 1087 | NE2 | HIS C | 134 | 19.532 | 13.127 | 35.710 | 1.00 | 247.23 |
| 1088 | C | HIS C | 134 | 19.573 | 14.664 | 31.255 | 1.00 | 77.87 |
| 1089 | O | HIS C | 134 | 19.843 | 15.338 | 30.252 | 1.00 | 77.87 |
| 1090 | N | ASN C | 135 | 19.539 | 15.167 | 32.484 | 1.00 | 77.12 |
| 1091 | CA | ASN C | 135 | 19.779 | 16.576 | 32.747 | 1.00 | 77.12 |
| 1092 | CB | ASN C | 135 | 18.442 | 17.308 | 32.868 | 1.00 | 227.67 |
| 1093 | CG | ASN C | 135 | 17.690 | 17.335 | 31.558 | 1.00 | 227.67 |
| 1094 | OD1 | ASN C | 135 | 18.287 | 17.624 | 30.520 | 1.00 | 227.67 |
| 1095 | ND2 | ASN C | 135 | 16.390 | 17.049 | 31.585 | 1.00 | 227.67 |
| 1096 | C | ASN C | 135 | 20.595 | 16.748 | 34.004 | 1.00 | 77.12 |
| 1097 | O | ASN C | 135 | 20.049 | 16.927 | 35.075 | 1.00 | 77.12 |
| 1098 | N | ILE C | 136 | 21.914 | 16.671 | 33.862 | 1.00 | 56.71 |
| 1099 | CA | ILE C | 136 | 22.855 | 16.810 | 34.974 | 1.00 | 56.71 |
| 1100 | CB | ILE C | 136 | 24.267 | 17.147 | 34.439 | 1.00 | 111.08 |
| 1101 | CG2 | ILE C | 136 | 24.215 | 18.298 | 33.468 | 1.00 | 111.08 |
| 1102 | CG1 | ILE C | 136 | 25.189 | 17.479 | 35.590 | 1.00 | 111.08 |
| 1103 | CD1 | ILE C | 136 | 26.605 | 17.684 | 35.143 | 1.00 | 111.08 |
| 1104 | C | ILE C | 136 | 22.387 | 17.870 | 35.956 | 1.00 | 56.71 |
| 1105 | O | ILE C | 136 | 22.276 | 19.044 | 35.623 | 1.00 | 56.71 |
| 1106 | N | SER C | 137 | 22.090 | 17.429 | 37.172 | 1.00 | 99.75 |
| 1107 | CA | SER C | 137 | 21.594 | 18.308 | 38.225 | 1.00 | 99.75 |
| 1108 | CB | SER C | 137 | 20.218 | 17.820 | 38.673 | 1.00 | 125.93 |
| 1109 | OG | SER C | 137 | 19.833 | 18.438 | 39.884 | 1.00 | 125.93 |
| 1110 | C | SER C | 137 | 22.517 | 18.411 | 39.437 | 1.00 | 99.75 |
| 1111 | O | SER C | 137 | 23.195 | 17.457 | 39.807 | 1.00 | 99.75 |
| 1112 | N | ILE C | 138 | 22.530 | 19.580 | 40.062 | 1.00 | 72.38 |
| 1113 | CA | ILE C | 138 | 23.366 | 19.816 | 41.235 | 1.00 | 72.38 |
| 1114 | CB | ILE C | 138 | 24.560 | 20.663 | 40.881 | 1.00 | 52.39 |
| 1115 | CG2 | ILE C | 138 | 25.241 | 21.106 | 42.161 | 1.00 | 52.39 |
| 1116 | CG1 | ILE C | 138 | 25.503 | 19.881 | 39.961 | 1.00 | 52.39 |
| 1117 | CD1 | ILE C | 138 | 26.574 | 20.747 | 39.311 | 1.00 | 52.39 |
| 1118 | C | ILE C | 138 | 22.609 | 20.556 | 42.335 | 1.00 | 72.38 |
| 1119 | O | ILE C | 138 | 22.109 | 21.667 | 42.112 | 1.00 | 72.38 |
| 1120 | N | THR C | 139 | 22.535 | 19.955 | 43.519 | 1.00 | 119.29 |
| 1121 | CA | THR C | 139 | 21.823 | 20.568 | 44.634 | 1.00 | 119.29 |
| 1122 | CB | THR C | 139 | 21.466 | 19.521 | 45.682 | 1.00 | 137.72 |
| 1123 | OG1 | THR C | 139 | 22.642 | 18.774 | 46.024 | 1.00 | 137.72 |
| 1124 | CG2 | THR C | 139 | 20.404 | 18.578 | 45.139 | 1.00 | 137.72 |
| 1125 | C | THR C | 139 | 22.679 | 21.655 | 45.275 | 1.00 | 119.29 |
| 1126 | O | THR C | 139 | 22.449 | 22.849 | 45.063 | 1.00 | 119.29 |
| 1127 | N | ASN C | 140 | 23.661 | 21.232 | 46.066 | 1.00 | 83.41 |
| 1128 | CA | ASN C | 140 | 24.585 | 22.147 | 46.730 | 1.00 | 83.41 |
| 1129 | CB | ASN C | 140 | 25.065 | 21.554 | 48.052 | 1.00 | 209.19 |
| 1130 | CG | ASN C | 140 | 26.123 | 22.402 | 48.714 | 1.00 | 209.19 |
| 1131 | OD1 | ASN C | 140 | 27.087 | 22.824 | 48.073 | 1.00 | 209.19 |
| 1132 | ND2 | ASN C | 140 | 25.950 | 22.645 | 50.006 | 1.00 | 209.19 |
| 1133 | C | ASN C | 140 | 25.758 | 22.277 | 45.780 | 1.00 | 83.41 |
| 1134 | O | ASN C | 140 | 26.403 | 21.277 | 45.455 | 1.00 | 83.41 |
| 1135 | N | ALA C | 141 | 26.037 | 23.503 | 45.343 | 1.00 | 87.00 |
| 1136 | CA | ALA C | 141 | 27.121 | 23.741 | 44.394 | 1.00 | 87.00 |
| 1137 | CB | ALA C | 141 | 26.704 | 24.753 | 43.381 | 1.00 | 58.59 |
| 1138 | C | ALA C | 141 | 28.440 | 24.162 | 44.994 | 1.00 | 87.00 |
| 1139 | O | ALA C | 141 | 28.527 | 25.112 | 45.753 | 1.00 | 87.00 |
| 1140 | N | THR C | 142 | 29.485 | 23.450 | 44.617 | 1.00 | 69.33 |
| 1141 | CA | THR C | 142 | 30.822 | 23.733 | 45.106 | 1.00 | 69.33 |
| 1142 | CB | THR C | 142 | 31.688 | 22.461 | 45.035 | 1.00 | 195.23 |
| 1143 | OG1 | THR C | 142 | 31.001 | 21.383 | 45.687 | 1.00 | 195.23 |
| 1144 | CG2 | THR C | 142 | 33.012 | 22.679 | 45.722 | 1.00 | 195.23 |
| 1145 | C | THR C | 142 | 31.388 | 24.815 | 44.202 | 1.00 | 69.33 |
| 1146 | O | THR C | 142 | 30.753 | 25.185 | 43.219 | 1.00 | 69.33 |
| 1147 | N | VAL C | 143 | 32.561 | 25.341 | 44.531 | 1.00 | 71.38 |
| 1148 | CA | VAL C | 143 | 33.154 | 26.387 | 43.699 | 1.00 | 71.38 |
| 1149 | CB | VAL C | 143 | 34.082 | 27.349 | 44.511 | 1.00 | 62.36 |
| 1150 | CG1 | VAL C | 143 | 35.270 | 26.572 | 45.059 | 1.00 | 62.36 |
| 1151 | CG2 | VAL C | 143 | 34.571 | 28.506 | 43.623 | 1.00 | 62.36 |
| 1152 | C | VAL C | 143 | 33.989 | 25.686 | 42.654 | 1.00 | 71.38 |
| 1153 | O | VAL C | 143 | 34.383 | 26.277 | 41.655 | 1.00 | 71.38 |
| 1154 | N | GLU C | 144 | 34.272 | 24.416 | 42.897 | 1.00 | 78.73 |
| 1155 | CA | GLU C | 144 | 35.065 | 23.659 | 41.954 | 1.00 | 78.73 |
| 1156 | CB | GLU C | 144 | 35.604 | 22.384 | 42.599 | 1.00 | 249.12 |
| 1157 | CG | GLU C | 144 | 36.574 | 22.653 | 43.732 | 1.00 | 249.12 |
| 1158 | CD | GLU C | 144 | 36.019 | 22.238 | 45.078 | 1.00 | 249.12 |
| 1159 | OE1 | GLU C | 144 | 35.711 | 21.040 | 45.239 | 1.00 | 249.12 |
| 1160 | OE2 | GLU C | 144 | 35.889 | 23.102 | 45.972 | 1.00 | 249.12 |
| 1161 | C | GLU C | 144 | 34.222 | 23.316 | 40.739 | 1.00 | 78.73 |
| 1162 | O | GLU C | 144 | 34.767 | 22.933 | 39.711 | 1.00 | 78.73 |
| 1163 | N | ASP C | 145 | 32.898 | 23.473 | 40.853 | 1.00 | 62.47 |
| 1164 | CA | ASP C | 145 | 31.977 | 23.174 | 39.754 | 1.00 | 62.47 |
| 1165 | CB | ASP C | 145 | 30.545 | 23.066 | 40.260 | 1.00 | 127.97 |
| 1166 | CG | ASP C | 145 | 30.305 | 21.798 | 41.028 | 1.00 | 127.97 |
| 1167 | OD1 | ASP C | 145 | 30.493 | 20.714 | 40.441 | 1.00 | 127.97 |
| 1168 | OD2 | ASP C | 145 | 29.935 | 21.883 | 42.217 | 1.00 | 127.97 |
| 1169 | C | ASP C | 145 | 32.053 | 24.220 | 38.663 | 1.00 | 62.47 |
| 1170 | O | ASP C | 145 | 31.548 | 24.011 | 37.568 | 1.00 | 62.47 |
| 1171 | N | SER C | 146 | 32.687 | 25.348 | 38.957 | 1.00 | 71.91 |
| 1172 | CA | SER C | 146 | 32.824 | 26.397 | 37.960 | 1.00 | 71.91 |
| 1173 | CB | SER C | 146 | 33.438 | 27.641 | 38.599 | 1.00 | 151.98 |
| 1174 | OG | SER C | 146 | 32.599 | 28.127 | 39.630 | 1.00 | 151.98 |
| 1175 | C | SER C | 146 | 33.711 | 25.866 | 36.837 | 1.00 | 71.91 |
| 1176 | O | SER C | 146 | 34.648 | 25.109 | 37.082 | 1.00 | 71.91 |
| 1177 | N | GLY C | 147 | 33.394 | 26.241 | 35.606 | 1.00 | 84.55 |
| 1178 | CA | GLY C | 147 | 34.170 | 25.785 | 34.466 | 1.00 | 84.55 |
| 1179 | C | GLY C | 147 | 33.449 | 26.062 | 33.158 | 1.00 | 84.55 |
| 1180 | O | GLY C | 147 | 32.552 | 26.898 | 33.121 | 1.00 | 84.55 |
| 1181 | N | THR C | 148 | 33.836 | 25.373 | 32.084 | 1.00 | 54.14 |
| 1182 | CA | THR C | 148 | 33.192 | 25.561 | 30.781 | 1.00 | 54.14 |
| 1183 | CB | THR C | 148 | 34.166 | 26.153 | 29.760 | 1.00 | 64.28 |
| 1184 | OG1 | THR C | 148 | 34.588 | 25.133 | 28.858 | 1.00 | 64.28 |
| 1185 | CG2 | THR C | 148 | 35.394 | 26.717 | 30.474 | 1.00 | 64.28 |
| 1186 | C | THR C | 148 | 32.671 | 24.216 | 30.285 | 1.00 | 54.14 |
| 1187 | O | THR C | 148 | 33.429 | 23.287 | 30.032 | 1.00 | 54.14 |
| 1188 | N | TYR C | 149 | 31.359 | 24.119 | 30.145 | 1.00 | 33.05 |
| 1189 | CA | TYR C | 149 | 30.725 | 22.879 | 29.753 | 1.00 | 33.05 |
| 1190 | CB | TYR C | 149 | 29.524 | 22.656 | 30.627 | 1.00 | 43.72 |
| 1191 | CG | TYR C | 149 | 29.787 | 22.522 | 32.108 | 1.00 | 43.72 |
| 1192 | CD1 | TYR C | 149 | 30.185 | 23.612 | 32.891 | 1.00 | 43.72 |
| 1193 | CE1 | TYR C | 149 | 30.359 | 23.470 | 34.266 | 1.00 | 43.72 |
| 1194 | CD2 | TYR C | 149 | 29.574 | 21.296 | 32.738 | 1.00 | 43.72 |
| 1195 | CE2 | TYR C | 149 | 29.739 | 21.143 | 34.095 | 1.00 | 43.72 |
| 1196 | CZ | TYR C | 149 | 30.130 | 22.222 | 34.876 | 1.00 | 43.72 |
| 1197 | OH | TYR C | 149 | 30.255 | 22.023 | 36.258 | 1.00 | 43.72 |
| 1198 | C | TYR C | 149 | 30.257 | 22.872 | 28.312 | 1.00 | 33.05 |
| 1199 | O | TYR C | 149 | 30.212 | 23.905 | 27.666 | 1.00 | 33.05 |
| 1200 | N | TYR C | 150 | 29.929 | 21.687 | 27.818 | 1.00 | 75.60 |
| 1201 | CA | TYR C | 150 | 29.402 | 21.473 | 26.481 | 1.00 | 75.60 |
| 1202 | CB | TYR C | 150 | 30.453 | 21.790 | 25.384 | 1.00 | 80.46 |
| 1203 | CG | TYR C | 150 | 31.554 | 20.780 | 25.087 | 1.00 | 80.46 |
| 1204 | CD1 | TYR C | 150 | 31.282 | 19.581 | 24.439 | 1.00 | 80.46 |
| 1205 | CE1 | TYR C | 150 | 32.288 | 18.664 | 24.162 | 1.00 | 80.46 |
| 1206 | CD2 | TYR C | 150 | 32.874 | 21.039 | 25.445 | 1.00 | 80.46 |
| 1207 | CE2 | TYR C | 150 | 33.889 | 20.130 | 25.168 | 1.00 | 80.46 |
| 1208 | CZ | TYR C | 150 | 33.590 | 18.938 | 24.529 | 1.00 | 80.46 |
| 1209 | OH | TYR C | 150 | 34.597 | 18.011 | 24.280 | 1.00 | 80.46 |
| 1210 | C | TYR C | 150 | 28.963 | 20.007 | 26.498 | 1.00 | 75.60 |
| 1211 | O | TYR C | 150 | 29.374 | 19.239 | 27.375 | 1.00 | 75.60 |
| 1212 | N | CYS C | 151 | 28.090 | 19.604 | 25.586 | 1.00 | 100.28 |
| 1213 | CA | CYS C | 151 | 27.657 | 18.211 | 25.572 | 1.00 | 100.28 |
| 1214 | C | CYS C | 151 | 27.674 | 17.617 | 24.167 | 1.00 | 100.28 |
| 1215 | O | CYS C | 151 | 27.634 | 18.349 | 23.191 | 1.00 | 100.28 |
| 1216 | CB | CYS C | 151 | 26.255 | 18.096 | 26.172 | 1.00 | 64.15 |
| 1217 | SG | CYS C | 151 | 24.959 | 19.135 | 25.424 | 1.00 | 64.15 |
| 1218 | N | THR C | 152 | 27.746 | 16.291 | 24.074 | 1.00 | 69.97 |
| 1219 | CA | THR C | 152 | 27.755 | 15.598 | 22.795 | 1.00 | 69.97 |
| 1220 | CB | THR C | 152 | 29.089 | 14.837 | 22.588 | 1.00 | 86.22 |
| 1221 | OG1 | THR C | 152 | 29.210 | 13.780 | 23.550 | 1.00 | 86.22 |
| 1222 | CG2 | THR C | 152 | 30.247 | 15.771 | 22.765 | 1.00 | 86.22 |
| 1223 | C | THR C | 152 | 26.594 | 14.610 | 22.813 | 1.00 | 69.97 |
| 1224 | O | THR C | 152 | 26.242 | 14.082 | 23.879 | 1.00 | 69.97 |
| 1225 | N | GLY C | 153 | 25.992 | 14.368 | 21.652 | 1.00 | 96.14 |
| 1226 | CA | GLY C | 153 | 24.879 | 13.441 | 21.597 | 1.00 | 96.14 |
| 1227 | C | GLY C | 153 | 24.583 | 13.010 | 20.182 | 1.00 | 96.14 |
| 1228 | O | GLY C | 153 | 25.037 | 13.663 | 19.247 | 1.00 | 96.14 |
| 1229 | N | LYS C | 154 | 23.845 | 11.918 | 20.017 | 1.00 | 77.66 |
| 1230 | CA | LYS C | 154 | 23.522 | 11.431 | 18.690 | 1.00 | 77.66 |
| 1231 | CB | LYS C | 154 | 23.722 | 9.916 | 18.603 | 1.00 | 222.01 |
| 1232 | CG | LYS C | 154 | 23.538 | 9.323 | 17.212 | 1.00 | 222.01 |
| 1233 | CD | LYS C | 154 | 23.820 | 7.833 | 17.253 | 1.00 | 222.01 |
| 1234 | CE | LYS C | 154 | 23.556 | 7.151 | 15.924 | 1.00 | 222.01 |
| 1235 | NZ | LYS C | 154 | 23.726 | 5.689 | 16.075 | 1.00 | 222.01 |
| 1236 | C | LYS C | 154 | 22.086 | 11.796 | 18.428 | 1.00 | 77.66 |
| 1237 | O | LYS C | 154 | 21.221 | 11.567 | 19.269 | 1.00 | 77.66 |
| 1238 | N | VAL C | 155 | 21.843 | 12.405 | 17.271 | 1.00 | 110.66 |
| 1239 | CA | VAL C | 155 | 20.496 | 12.796 | 16.862 | 1.00 | 110.66 |
| 1240 | CB | VAL C | 155 | 20.397 | 14.288 | 16.589 | 1.00 | 77.82 |
| 1241 | CG1 | VAL C | 155 | 18.985 | 14.643 | 16.163 | 1.00 | 77.82 |
| 1242 | CG2 | VAL C | 155 | 20.778 | 15.046 | 17.834 | 1.00 | 77.82 |
| 1243 | C | VAL C | 155 | 20.208 | 12.046 | 15.579 | 1.00 | 110.66 |
| 1244 | O | VAL C | 155 | 21.000 | 12.085 | 14.629 | 1.00 | 110.66 |
| 1245 | N | TRP C | 156 | 19.074 | 11.363 | 15.553 | 1.00 | 192.10 |
| 1246 | CA | TRP C | 156 | 18.727 | 10.560 | 14.401 | 1.00 | 192.10 |
| 1247 | CB | TRP C | 156 | 18.811 | 11.362 | 13.120 | 1.00 | 246.44 |
| 1248 | CG | TRP C | 156 | 17.823 | 12.399 | 13.086 | 1.00 | 246.44 |
| 1249 | CD2 | TRP C | 156 | 16.417 | 12.222 | 13.206 | 1.00 | 246.44 |
| 1250 | CE2 | TRP C | 156 | 15.830 | 13.503 | 13.150 | 1.00 | 246.44 |
| 1251 | CE3 | TRP C | 156 | 15.592 | 11.100 | 13.355 | 1.00 | 246.44 |
| 1252 | CD1 | TRP C | 156 | 18.042 | 13.729 | 12.960 | 1.00 | 246.44 |
| 1253 | NE1 | TRP C | 156 | 16.846 | 14.407 | 13.005 | 1.00 | 246.44 |
| 1254 | CZ2 | TRP C | 156 | 14.454 | 13.698 | 13.239 | 1.00 | 246.44 |
| 1255 | CZ3 | TRP C | 156 | 14.217 | 11.293 | 13.438 | 1.00 | 246.44 |
| 1256 | CH2 | TRP C | 156 | 13.662 | 12.588 | 13.378 | 1.00 | 246.44 |
| 1257 | C | TRP C | 156 | 19.771 | 9.496 | 14.335 | 1.00 | 192.10 |
| 1258 | O | TRP C | 156 | 19.673 | 8.478 | 15.006 | 1.00 | 192.10 |
| 1259 | N | GLN C | 157 | 20.800 | 9.769 | 13.545 | 1.00 | 118.64 |
| 1260 | CA | GLN C | 157 | 21.861 | 8.814 | 13.373 | 1.00 | 118.64 |
| 1261 | CB | GLN C | 157 | 21.511 | 7.931 | 12.180 | 1.00 | 249.64 |
| 1262 | CG | GLN C | 157 | 20.361 | 6.995 | 12.528 | 1.00 | 249.64 |
| 1263 | CD | GLN C | 157 | 20.670 | 6.255 | 13.803 | 1.00 | 249.64 |
| 1264 | OE1 | GLN C | 157 | 21.748 | 5.725 | 13.918 | 1.00 | 249.64 |
| 1265 | NE2 | GLN C | 157 | 19.770 | 6.241 | 14.761 | 1.00 | 249.64 |
| 1266 | C | GLN C | 157 | 23.230 | 9.430 | 13.234 | 1.00 | 118.64 |
| 1267 | O | GLN C | 157 | 24.183 | 8.768 | 12.826 | 1.00 | 118.64 |
| 1268 | N | LEU C | 158 | 23.323 | 10.707 | 13.574 | 1.00 | 91.98 |
| 1269 | CA | LEU C | 158 | 24.600 | 11.404 | 13.510 | 1.00 | 91.98 |
| 1270 | CB | LEU C | 158 | 24.580 | 12.461 | 12.418 | 1.00 | 164.15 |
| 1271 | CG | LEU C | 158 | 24.775 | 11.991 | 10.980 | 1.00 | 164.15 |
| 1272 | CD1 | LEU C | 158 | 25.736 | 12.973 | 10.359 | 1.00 | 164.15 |
| 1273 | CD2 | LEU C | 158 | 25.383 | 10.614 | 10.868 | 1.00 | 164.15 |
| 1274 | C | LEU C | 158 | 25.000 | 12.046 | 14.841 | 1.00 | 91.98 |
| 1275 | O | LEU C | 158 | 24.147 | 12.397 | 15.658 | 1.00 | 91.98 |
| 1276 | N | ASP C | 159 | 26.307 | 12.190 | 15.047 | 1.00 | 113.11 |
| 1277 | CA | ASP C | 159 | 26.839 | 12.762 | 16.273 | 1.00 | 113.11 |
| 1278 | CB | ASP C | 159 | 28.230 | 12.173 | 16.564 | 1.00 | 249.37 |
| 1279 | CG | ASP C | 159 | 28.236 | 10.645 | 16.599 | 1.00 | 249.37 |
| 1280 | OD1 | ASP C | 159 | 27.536 | 10.057 | 17.451 | 1.00 | 249.37 |
| 1281 | OD2 | ASP C | 159 | 28.949 | 10.033 | 15.771 | 1.00 | 249.37 |
| 1282 | C | ASP C | 159 | 26.939 | 14.280 | 16.148 | 1.00 | 113.11 |
| 1283 | O | ASP C | 159 | 27.223 | 14.788 | 15.063 | 1.00 | 113.11 |
| 1284 | N | TYR C | 160 | 26.701 | 14.995 | 17.254 | 1.00 | 103.02 |
| 1285 | CA | TYR C | 160 | 26.782 | 16.464 | 17.278 | 1.00 | 103.02 |
| 1286 | CB | TYR C | 160 | 25.424 | 17.105 | 17.057 | 1.00 | 177.48 |
| 1287 | CG | TYR C | 160 | 24.711 | 16.622 | 15.826 | 1.00 | 177.48 |
| 1288 | CD1 | TYR C | 160 | 23.860 | 15.521 | 15.882 | 1.00 | 177.48 |
| 1289 | CE1 | TYR C | 160 | 23.204 | 15.062 | 14.752 | 1.00 | 177.48 |
| 1290 | CD2 | TYR C | 160 | 24.891 | 17.255 | 14.600 | 1.00 | 177.48 |
| 1291 | CE2 | TYR C | 160 | 24.241 | 16.804 | 13.458 | 1.00 | 177.48 |
| 1292 | CZ | TYR C | 160 | 23.400 | 15.707 | 13.545 | 1.00 | 177.48 |
| 1293 | OH | TYR C | 160 | 22.763 | 15.245 | 12.427 | 1.00 | 177.48 |
| 1294 | C | TYR C | 160 | 27.333 | 16.998 | 18.581 | 1.00 | 103.02 |
| 1295 | O | TYR C | 160 | 27.095 | 16.449 | 19.647 | 1.00 | 103.02 |
| 1296 | N | GLU C | 161 | 28.052 | 18.102 | 18.474 | 1.00 | 74.72 |
| 1297 | CA | GLU C | 161 | 28.687 | 18.775 | 19.599 | 1.00 | 74.72 |
| 1298 | CB | GLU C | 161 | 30.170 | 18.972 | 19.273 | 1.00 | 249.14 |
| 1299 | CG | GLU C | 161 | 30.978 | 19.729 | 20.300 | 1.00 | 249.14 |
| 1300 | CD | GLU C | 161 | 32.456 | 19.531 | 20.085 | 1.00 | 249.14 |
| 1301 | OE1 | GLU C | 161 | 33.259 | 20.350 | 20.582 | 1.00 | 249.14 |
| 1302 | OE2 | GLU C | 161 | 32.808 | 18.538 | 19.417 | 1.00 | 249.14 |
| 1303 | C | GLU C | 161 | 27.985 | 20.117 | 19.779 | 1.00 | 74.72 |
| 1304 | O | GLU C | 161 | 27.656 | 20.780 | 18.806 | 1.00 | 74.72 |
| 1305 | N | SER C | 162 | 27.743 | 20.516 | 21.021 | 1.00 | 59.53 |
| 1306 | CA | SER C | 162 | 27.055 | 21.776 | 21.320 | 1.00 | 59.53 |
| 1307 | CB | SER C | 162 | 26.210 | 21.627 | 22.573 | 1.00 | 71.52 |
| 1308 | OG | SER C | 162 | 27.017 | 21.253 | 23.673 | 1.00 | 71.52 |
| 1309 | C | SER C | 162 | 28.038 | 22.914 | 21.532 | 1.00 | 59.53 |
| 1310 | O | SER C | 162 | 29.247 | 22.679 | 21.608 | 1.00 | 59.53 |
| 1311 | N | GLU C | 163 | 27.521 | 24.145 | 21.615 | 1.00 | 68.20 |
| 1312 | CA | GLU C | 163 | 28.371 | 25.330 | 21.810 | 1.00 | 68.20 |
| 1313 | CB | GLU C | 163 | 27.580 | 26.610 | 21.565 | 1.00 | 172.64 |
| 1314 | CG | GLU C | 163 | 27.289 | 26.902 | 20.098 | 1.00 | 172.64 |
| 1315 | CD | GLU C | 163 | 28.513 | 27.375 | 19.334 | 1.00 | 172.64 |
| 1316 | OE1 | GLU C | 163 | 29.120 | 28.383 | 19.757 | 1.00 | 172.64 |
| 1317 | OE2 | GLU C | 163 | 28.864 | 26.748 | 18.311 | 1.00 | 172.64 |
| 1318 | C | GLU C | 163 | 28.856 | 25.296 | 23.246 | 1.00 | 68.20 |
| 1319 | O | GLU C | 163 | 28.104 | 24.920 | 24.131 | 1.00 | 68.20 |
| 1320 | N | PRO C | 164 | 30.123 | 25.683 | 23.498 | 1.00 | 54.78 |
| 1321 | CD | PRO C | 164 | 31.163 | 26.188 | 22.601 | 1.00 | 96.83 |
| 1322 | CA | PRO C | 164 | 30.609 | 25.645 | 24.876 | 1.00 | 54.78 |
| 1323 | CB | PRO C | 164 | 32.109 | 25.851 | 24.709 | 1.00 | 96.83 |
| 1324 | CG | PRO C | 164 | 32.176 | 26.765 | 23.584 | 1.00 | 96.83 |
| 1325 | C | PRO C | 164 | 29.950 | 26.720 | 25.688 | 1.00 | 54.78 |
| 1326 | O | PRO C | 164 | 29.480 | 27.695 | 25.137 | 1.00 | 54.78 |
| 1327 | N | LEU C | 165 | 29.898 | 26.537 | 27.000 | 1.00 | 73.43 |
| 1328 | CA | LEU C | 165 | 29.272 | 27.513 | 27.861 | 1.00 | 73.43 |
| 1329 | CB | LEU C | 165 | 27.829 | 27.112 | 28.127 | 1.00 | 54.02 |
| 1330 | CG | LEU C | 165 | 27.163 | 27.955 | 29.201 | 1.00 | 54.02 |
| 1331 | CD1 | LEU C | 165 | 27.395 | 29.384 | 28.820 | 1.00 | 54.02 |
| 1332 | CD2 | LEU C | 165 | 25.690 | 27.665 | 29.329 | 1.00 | 54.02 |
| 1333 | C | LEU C | 165 | 30.010 | 27.634 | 29.164 | 1.00 | 73.43 |
| 1334 | O | LEU C | 165 | 30.200 | 26.641 | 29.859 | 1.00 | 73.43 |
| 1335 | N | ASN C | 166 | 30.420 | 28.853 | 29.493 | 1.00 | 53.84 |
| 1336 | CA | ASN C | 166 | 31.148 | 29.098 | 30.736 | 1.00 | 53.84 |
| 1337 | CB | ASN C | 166 | 31.979 | 30.368 | 30.646 | 1.00 | 80.99 |
| 1338 | CG | ASN C | 166 | 33.392 | 30.110 | 30.181 | 1.00 | 80.99 |
| 1339 | OD1 | ASN C | 166 | 33.956 | 29.054 | 30.431 | 1.00 | 80.99 |
| 1340 | ND2 | ASN C | 166 | 33.973 | 31.096 | 29.516 | 1.00 | 80.99 |
| 1341 | C | ASN C | 166 | 30.233 | 29.236 | 31.918 | 1.00 | 53.84 |
| 1342 | O | ASN C | 166 | 29.145 | 29.756 | 31.789 | 1.00 | 53.84 |
| 1343 | N | ILE C | 167 | 30.693 | 28.806 | 33.084 | 1.00 | 65.33 |
| 1344 | CA | ILE C | 167 | 29.878 | 28.881 | 34.292 | 1.00 | 65.33 |
| 1345 | CB | ILE C | 167 | 29.218 | 27.527 | 34.603 | 1.00 | 38.60 |
| 1346 | CG2 | ILE C | 167 | 28.736 | 27.503 | 36.027 | 1.00 | 38.60 |
| 1347 | CG1 | ILE C | 167 | 28.066 | 27.269 | 33.618 | 1.00 | 38.60 |
| 1348 | CD1 | ILE C | 167 | 27.261 | 26.070 | 33.933 | 1.00 | 38.60 |
| 1349 | C | ILE C | 167 | 30.717 | 29.258 | 35.475 | 1.00 | 65.33 |
| 1350 | O | ILE C | 167 | 31.781 | 28.681 | 35.694 | 1.00 | 65.33 |
| 1351 | N | THR C | 168 | 30.248 | 30.223 | 36.246 | 1.00 | 58.90 |
| 1352 | CA | THR C | 168 | 31.015 | 30.619 | 37.406 | 1.00 | 58.90 |
| 1353 | CB | THR C | 168 | 31.537 | 32.023 | 37.260 | 1.00 | 100.28 |
| 1354 | OG1 | THR C | 168 | 32.315 | 32.110 | 36.067 | 1.00 | 100.28 |
| 1355 | CG2 | THR C | 168 | 32.406 | 32.354 | 38.438 | 1.00 | 100.28 |
| 1356 | C | THR C | 168 | 30.230 | 30.502 | 38.708 | 1.00 | 58.90 |
| 1357 | O | THR C | 168 | 29.042 | 30.852 | 38.795 | 1.00 | 58.90 |
| 1358 | N | VAL C | 169 | 30.908 | 29.965 | 39.708 | 1.00 | 74.23 |
| 1359 | CA | VAL C | 169 | 30.337 | 29.795 | 41.021 | 1.00 | 74.23 |
| 1360 | CB | VAL C | 169 | 30.424 | 28.330 | 41.467 | 1.00 | 86.72 |
| 1361 | CG1 | VAL C | 169 | 30.314 | 28.229 | 42.962 | 1.00 | 86.72 |
| 1362 | CG2 | VAL C | 169 | 29.323 | 27.547 | 40.813 | 1.00 | 86.72 |
| 1363 | C | VAL C | 169 | 31.196 | 30.668 | 41.924 | 1.00 | 74.23 |
| 1364 | O | VAL C | 169 | 32.359 | 30.328 | 42.184 | 1.00 | 74.23 |
| 1365 | N | ILE C | 170 | 30.645 | 31.805 | 42.365 | 1.00 | 66.08 |
| 1366 | CA | ILE C | 170 | 31.376 | 32.711 | 43.252 | 1.00 | 66.08 |
| 1367 | CB | ILE C | 170 | 30.995 | 34.166 | 42.997 | 1.00 | 82.85 |
| 1368 | CG2 | ILE C | 170 | 31.079 | 34.476 | 41.520 | 1.00 | 82.85 |
| 1369 | CG1 | ILE C | 170 | 29.572 | 34.412 | 43.431 | 1.00 | 82.85 |
| 1370 | CD1 | ILE C | 170 | 29.097 | 35.848 | 43.156 | 1.00 | 82.85 |
| 1371 | C | ILE C | 170 | 31.092 | 32.355 | 44.701 | 1.00 | 66.08 |
| 1372 | O | ILE C | 170 | 30.272 | 31.482 | 44.960 | 1.00 | 66.08 |
| 1373 | N | LYS C | 171 | 31.771 | 32.999 | 45.644 | 1.00 | 110.58 |
| 1374 | CA | LYS C | 171 | 31.545 | 32.688 | 47.052 | 1.00 | 110.58 |
| 1375 | CB | LYS C | 171 | 32.749 | 31.935 | 47.625 | 1.00 | 192.81 |
| 1376 | CG | LYS C | 171 | 34.062 | 32.679 | 47.478 | 1.00 | 192.81 |
| 1377 | CD | LYS C | 171 | 35.247 | 31.723 | 47.505 | 1.00 | 192.81 |
| 1378 | CE | LYS C | 171 | 35.319 | 30.934 | 48.803 | 1.00 | 192.81 |
| 1379 | NZ | LYS C | 171 | 36.464 | 29.978 | 48.796 | 1.00 | 192.81 |
| 1380 | C | LYS C | 171 | 31.263 | 33.931 | 47.882 | 1.00 | 110.58 |
| 1381 | O | LYS C | 171 | 30.884 | 33.830 | 49.050 | 1.00 | 110.58 |
| 1382 | C1 | NAG C | 221 | 4.609 | 28.125 | 21.539 | 1.00 | 248.09 |
| 1383 | C2 | NAG C | 221 | 4.738 | 26.611 | 21.473 | 1.00 | 248.09 |
| 1384 | N2 | NAG C | 221 | 6.129 | 26.254 | 21.269 | 1.00 | 248.09 |
| 1385 | C7 | NAG C | 221 | 6.578 | 25.075 | 21.680 | 1.00 | 248.09 |
| 1386 | O7 | NAG C | 221 | 5.867 | 24.254 | 22.257 | 1.00 | 248.09 |
| 1387 | C8 | NAG C | 221 | 8.042 | 24.762 | 21.420 | 1.00 | 248.09 |
| 1388 | C3 | NAG C | 221 | 3.908 | 26.047 | 20.327 | 1.00 | 248.09 |
| 1389 | O3 | NAG C | 221 | 3.902 | 24.630 | 20.401 | 1.00 | 248.09 |
| 1390 | C4 | NAG C | 221 | 2.465 | 26.559 | 20.341 | 1.00 | 248.09 |
| 1391 | O4 | NAG C | 221 | 1.852 | 26.163 | 19.095 | 1.00 | 248.09 |
| 1392 | C5 | NAG C | 221 | 2.447 | 28.096 | 20.488 | 1.00 | 248.09 |
| 1393 | O5 | NAG C | 221 | 3.226 | 28.499 | 21.641 | 1.00 | 248.09 |
| 1394 | C6 | NAG C | 221 | 1.052 | 28.659 | 20.692 | 1.00 | 248.09 |
| 1395 | O6 | NAG C | 221 | 0.460 | 28.142 | 21.875 | 1.00 | 248.09 |
| 1396 | C1 | NAG C | 222 | 0.468 | 26.179 | 18.986 | 1.00 | 248.99 |
| 1397 | C2 | NAG C | 222 | −0.014 | 24.897 | 18.283 | 1.00 | 248.99 |
| 1398 | N2 | NAG C | 222 | 0.382 | 23.729 | 19.048 | 1.00 | 248.99 |
| 1399 | C7 | NAG C | 222 | −0.541 | 22.909 | 19.543 | 1.00 | 248.99 |
| 1400 | O7 | NAG C | 222 | −1.750 | 23.088 | 19.392 | 1.00 | 248.99 |
| 1401 | C8 | NAG C | 222 | −0.046 | 21.704 | 20.330 | 1.00 | 248.99 |
| 1402 | C3 | NAG C | 222 | 0.566 | 24.815 | 16.861 | 1.00 | 248.99 |
| 1403 | O3 | NAG C | 222 | −0.012 | 23.714 | 16.171 | 1.00 | 248.99 |
| 1404 | C4 | NAG C | 222 | 0.292 | 26.112 | 16.083 | 1.00 | 248.99 |
| 1405 | O4 | NAG C | 222 | 0.989 | 26.082 | 14.843 | 1.00 | 248.99 |
| 1406 | C5 | NAG C | 222 | 0.742 | 27.337 | 16.897 | 1.00 | 248.99 |
| 1407 | O5 | NAG C | 222 | 0.107 | 27.330 | 18.201 | 1.00 | 248.99 |
| 1408 | C6 | NAG C | 222 | 0.396 | 28.661 | 16.232 | 1.00 | 248.99 |
| 1409 | O6 | NAG C | 222 | 1.499 | 29.556 | 16.260 | 1.00 | 248.99 |
| 1410 | C1 | NAG C | 242 | 18.858 | 43.706 | 21.097 | 1.00 | 98.91 |
| 1411 | C2 | NAG C | 242 | 18.159 | 43.460 | 19.760 | 1.00 | 98.91 |
| 1412 | N2 | NAG C | 242 | 16.728 | 43.568 | 19.914 | 1.00 | 98.91 |
| 1413 | C7 | NAG C | 242 | 16.062 | 44.435 | 19.166 | 1.00 | 98.91 |
| 1414 | O7 | NAG C | 242 | 16.610 | 45.163 | 18.336 | 1.00 | 98.91 |
| 1415 | C8 | NAG C | 242 | 14.561 | 44.512 | 19.366 | 1.00 | 98.91 |
| 1416 | C3 | NAG C | 242 | 18.507 | 42.075 | 19.237 | 1.00 | 98.91 |
| 1417 | O3 | NAG C | 242 | 17.925 | 41.880 | 17.955 | 1.00 | 98.91 |
| 1418 | C4 | NAG C | 242 | 20.020 | 41.925 | 19.144 | 1.00 | 98.91 |
| 1419 | O4 | NAG C | 242 | 20.340 | 40.556 | 18.833 | 1.00 | 98.91 |
| 1420 | C5 | NAG C | 242 | 20.708 | 42.318 | 20.459 | 1.00 | 98.91 |
| 1421 | O5 | NAG C | 242 | 20.270 | 43.615 | 20.916 | 1.00 | 98.91 |
| 1422 | C6 | NAG C | 242 | 22.196 | 42.434 | 20.243 | 1.00 | 98.91 |
| 1423 | O6 | NAG C | 242 | 22.917 | 41.643 | 21.170 | 1.00 | 98.91 |
| 1424 | C1 | NAG C | 243 | 20.966 | 40.334 | 17.621 | 1.00 | 148.54 |
| 1425 | C2 | NAG C | 243 | 21.805 | 39.050 | 17.674 | 1.00 | 148.54 |
| 1426 | N2 | NAG C | 243 | 22.863 | 39.159 | 18.662 | 1.00 | 148.54 |
| 1427 | C7 | NAG C | 243 | 23.081 | 38.154 | 19.504 | 1.00 | 148.54 |
| 1428 | O7 | NAG C | 243 | 22.402 | 37.126 | 19.506 | 1.00 | 148.54 |
| 1429 | C8 | NAG C | 243 | 24.212 | 38.320 | 20.503 | 1.00 | 148.54 |
| 1430 | C3 | NAG C | 243 | 22.422 | 38.803 | 16.299 | 1.00 | 148.54 |
| 1431 | O3 | NAG C | 243 | 23.126 | 37.573 | 16.300 | 1.00 | 148.54 |
| 1432 | C4 | NAG C | 243 | 21.341 | 38.791 | 15.201 | 1.00 | 148.54 |
| 1433 | O4 | NAG C | 243 | 21.974 | 38.713 | 13.890 | 1.00 | 148.54 |
| 1434 | C5 | NAG C | 243 | 20.529 | 40.090 | 15.296 | 1.00 | 148.54 |
| 1435 | O5 | NAG C | 243 | 19.954 | 40.216 | 16.611 | 1.00 | 148.54 |
| 1436 | C6 | NAG C | 243 | 19.402 | 40.197 | 14.299 | 1.00 | 148.54 |
| 1437 | O6 | NAG C | 243 | 18.380 | 39.264 | 14.597 | 1.00 | 148.54 |
| 1438 | C1 | MAN C | 244 | 21.585 | 37.818 | 12.938 | 1.00 | 182.20 |
| 1439 | C2 | MAN C | 244 | 21.654 | 36.312 | 13.272 | 1.00 | 182.20 |
| 1440 | O2 | MAN C | 244 | 20.383 | 35.858 | 13.660 | 1.00 | 182.20 |
| 1441 | C3 | MAN C | 244 | 22.042 | 35.694 | 11.892 | 1.00 | 182.20 |
| 1442 | O3 | MAN C | 244 | 22.157 | 34.284 | 11.945 | 1.00 | 182.20 |
| 1443 | C4 | MAN C | 244 | 21.095 | 36.131 | 10.730 | 1.00 | 182.20 |
| 1444 | O4 | MAN C | 244 | 21.496 | 35.520 | 9.503 | 1.00 | 182.20 |
| 1445 | C5 | MAN C | 244 | 21.199 | 37.666 | 10.607 | 1.00 | 182.20 |
| 1446 | O5 | MAN C | 244 | 20.771 | 38.312 | 11.834 | 1.00 | 182.20 |
| 1447 | C6 | MAN C | 244 | 20.464 | 38.264 | 9.406 | 1.00 | 182.20 |
| 1448 | O6 | MAN C | 244 | 19.092 | 38.434 | 9.670 | 1.00 | 182.20 |
| 1449 | C1 | NAG C | 250 | −1.001 | 38.689 | 31.557 | 1.00 | 249.77 |
| 1450 | C2 | NAG C | 250 | −1.761 | 37.609 | 32.354 | 1.00 | 249.77 |
| 1451 | N2 | NAG C | 250 | −1.602 | 37.821 | 33.782 | 1.00 | 249.77 |
| 1452 | C7 | NAG C | 250 | −2.636 | 38.209 | 34.526 | 1.00 | 249.77 |
| 1453 | O7 | NAG C | 250 | −3.761 | 38.414 | 34.060 | 1.00 | 249.77 |
| 1454 | C8 | NAG C | 250 | −2.384 | 38.404 | 36.016 | 1.00 | 249.77 |
| 1455 | C3 | NAG C | 250 | −1.221 | 36.224 | 31.975 | 1.00 | 249.77 |
| 1456 | O3 | NAG C | 250 | −1.975 | 35.209 | 32.626 | 1.00 | 249.77 |
| 1457 | C4 | NAG C | 250 | −1.287 | 36.028 | 30.458 | 1.00 | 249.77 |
| 1458 | O4 | NAG C | 250 | −0.662 | 34.799 | 30.113 | 1.00 | 249.77 |
| 1459 | C5 | NAG C | 250 | −0.582 | 37.194 | 29.736 | 1.00 | 249.77 |
| 1460 | O5 | NAG C | 250 | −1.150 | 38.457 | 30.150 | 1.00 | 249.77 |
| 1461 | C6 | NAG C | 250 | −0.717 | 37.121 | 28.224 | 1.00 | 249.77 |
| 1462 | O6 | NAG C | 250 | −0.351 | 38.351 | 27.612 | 1.00 | 249.77 |
| 1463 | C1 | NAG C | 274 | 16.034 | 53.837 | 43.921 | 1.00 | 248.46 |
| 1464 | C2 | NAG C | 274 | 17.088 | 53.346 | 44.921 | 1.00 | 248.46 |
| 1465 | N2 | NAG C | 274 | 16.465 | 52.511 | 45.928 | 1.00 | 248.46 |
| 1466 | C7 | NAG C | 274 | 17.189 | 51.604 | 46.575 | 1.00 | 248.46 |
| 1467 | O7 | NAG C | 274 | 18.387 | 51.422 | 46.354 | 1.00 | 248.46 |
| 1468 | C8 | NAG C | 274 | 16.474 | 50.767 | 47.625 | 1.00 | 248.46 |
| 1469 | C3 | NAG C | 274 | 17.768 | 54.539 | 45.598 | 1.00 | 248.46 |
| 1470 | O3 | NAG C | 274 | 18.835 | 54.081 | 46.416 | 1.00 | 248.46 |
| 1471 | C4 | NAG C | 274 | 18.306 | 55.518 | 44.553 | 1.00 | 248.46 |
| 1472 | O4 | NAG C | 274 | 18.793 | 56.685 | 45.202 | 1.00 | 248.46 |
| 1473 | C5 | NAG C | 274 | 17.195 | 55.898 | 43.563 | 1.00 | 248.46 |
| 1474 | O5 | NAG C | 274 | 16.641 | 54.710 | 42.959 | 1.00 | 248.46 |
| 1475 | C6 | NAG C | 274 | 17.688 | 56.784 | 42.432 | 1.00 | 248.46 |
| 1476 | O6 | NAG C | 274 | 16.703 | 56.920 | 41.418 | 1.00 | 248.46 |
| 1477 | C1 | NAG C | 335 | 15.450 | 18.012 | 31.039 | 1.00 | 249.77 |
| 1478 | C2 | NAG C | 335 | 14.351 | 18.418 | 32.049 | 1.00 | 249.77 |
| 1479 | N2 | NAG C | 335 | 14.844 | 18.144 | 33.387 | 1.00 | 249.77 |
| 1480 | C7 | NAG C | 335 | 15.027 | 19.131 | 34.258 | 1.00 | 249.77 |
| 1481 | O7 | NAG C | 335 | 14.782 | 20.312 | 34.004 | 1.00 | 249.77 |
| 1482 | C8 | NAG C | 335 | 15.555 | 18.743 | 35.627 | 1.00 | 249.77 |
| 1483 | C3 | NAG C | 335 | 13.010 | 17.686 | 31.860 | 1.00 | 249.77 |
| 1484 | O3 | NAG C | 335 | 11.981 | 18.411 | 32.519 | 1.00 | 249.77 |
| 1485 | C4 | NAG C | 335 | 12.654 | 17.546 | 30.386 | 1.00 | 249.77 |
| 1486 | O4 | NAG C | 335 | 11.455 | 16.796 | 30.245 | 1.00 | 249.77 |
| 1487 | C5 | NAG C | 335 | 13.801 | 16.839 | 29.679 | 1.00 | 249.77 |
| 1488 | O5 | NAG C | 335 | 14.974 | 17.683 | 29.710 | 1.00 | 249.77 |
| 1489 | C6 | NAG C | 335 | 13.481 | 16.566 | 28.214 | 1.00 | 249.77 |
| 1490 | O6 | NAG C | 335 | 13.512 | 15.176 | 27.922 | 1.00 | 249.77 |
| 1491 | C1 | NAG C | 340 | 26.860 | 22.059 | 50.969 | 1.00 | 249.77 |
| 1492 | C2 | NAG C | 340 | 27.612 | 23.165 | 51.681 | 1.00 | 249.77 |
| 1493 | N2 | NAG C | 340 | 28.257 | 24.040 | 50.724 | 1.00 | 249.77 |
| 1494 | C7 | NAG C | 340 | 28.068 | 25.353 | 50.821 | 1.00 | 249.77 |
| 1495 | O7 | NAG C | 340 | 27.368 | 25.865 | 51.703 | 1.00 | 249.77 |
| 1496 | C8 | NAG C | 340 | 28.755 | 26.232 | 49.794 | 1.00 | 249.77 |
| 1497 | C3 | NAG C | 340 | 28.630 | 22.560 | 52.634 | 1.00 | 249.77 |
| 1498 | O3 | NAG C | 340 | 29.275 | 23.608 | 53.354 | 1.00 | 249.77 |
| 1499 | C4 | NAG C | 340 | 27.915 | 21.620 | 53.612 | 1.00 | 249.77 |
| 1500 | O4 | NAG C | 340 | 28.896 | 20.922 | 54.365 | 1.00 | 249.77 |
| 1501 | C5 | NAG C | 340 | 26.987 | 20.611 | 52.880 | 1.00 | 249.77 |
| 1502 | O5 | NAG C | 340 | 26.141 | 21.281 | 51.923 | 1.00 | 249.77 |
| 1503 | C6 | NAG C | 340 | 26.045 | 19.869 | 53.817 | 1.00 | 249.77 |
| 1504 | O6 | NAG C | 340 | 24.805 | 19.571 | 53.193 | 1.00 | 249.77 |
| 1505 | C1 | NAG C | 366 | 35.293 | 30.923 | 28.965 | 1.00 | 158.36 |
| 1506 | C2 | NAG C | 366 | 35.391 | 31.732 | 27.687 | 1.00 | 158.36 |
| 1507 | N2 | NAG C | 366 | 34.394 | 31.261 | 26.748 | 1.00 | 158.36 |
| 1508 | C7 | NAG C | 366 | 33.197 | 31.835 | 26.713 | 1.00 | 158.36 |
| 1509 | O7 | NAG C | 366 | 32.885 | 32.778 | 27.446 | 1.00 | 158.36 |
| 1510 | C8 | NAG C | 366 | 32.191 | 31.285 | 25.707 | 1.00 | 158.36 |
| 1511 | C3 | NAG C | 366 | 36.780 | 31.584 | 27.089 | 1.00 | 158.36 |
| 1512 | O3 | NAG C | 366 | 36.910 | 32.461 | 25.981 | 1.00 | 158.36 |
| 1513 | C4 | NAG C | 366 | 37.866 | 31.903 | 28.119 | 1.00 | 158.36 |
| 1514 | O4 | NAG C | 366 | 39.144 | 31.523 | 27.573 | 1.00 | 158.36 |
| 1515 | C5 | NAG C | 366 | 37.620 | 31.138 | 29.429 | 1.00 | 158.36 |
| 1516 | O5 | NAG C | 366 | 36.277 | 31.367 | 29.896 | 1.00 | 158.36 |
| 1517 | C6 | NAG C | 366 | 38.550 | 31.570 | 30.549 | 1.00 | 158.36 |
| 1518 | O6 | NAG C | 366 | 38.325 | 30.807 | 31.727 | 1.00 | 158.36 |
| 1519 | C1 | NAG C | 367 | 40.136 | 32.494 | 27.559 | 1.00 | 249.59 |
| 1520 | C2 | NAG C | 367 | 41.511 | 31.828 | 27.487 | 1.00 | 249.59 |
| 1521 | N2 | NAG C | 367 | 41.702 | 30.934 | 28.613 | 1.00 | 249.59 |
| 1522 | C7 | NAG C | 367 | 41.695 | 29.619 | 28.418 | 1.00 | 249.59 |
| 1523 | O7 | NAG C | 367 | 41.532 | 29.106 | 27.308 | 1.00 | 249.59 |
| 1524 | C8 | NAG C | 367 | 41.899 | 28.735 | 29.639 | 1.00 | 249.59 |
| 1525 | C3 | NAG C | 367 | 42.590 | 32.914 | 27.465 | 1.00 | 249.59 |
| 1526 | O3 | NAG C | 367 | 43.877 | 32.321 | 27.352 | 1.00 | 249.59 |
| 1527 | C4 | NAG C | 367 | 42.343 | 33.850 | 26.278 | 1.00 | 249.59 |
| 1528 | O4 | NAG C | 367 | 43.281 | 34.917 | 26.303 | 1.00 | 249.59 |
| 1529 | C5 | NAG C | 367 | 40.913 | 34.411 | 26.335 | 1.00 | 249.59 |
| 1530 | O5 | NAG C | 367 | 39.945 | 33.331 | 26.405 | 1.00 | 249.59 |
| 1531 | C6 | NAG C | 367 | 40.576 | 35.245 | 25.112 | 1.00 | 249.59 |
| 1532 | O6 | NAG C | 367 | 39.610 | 34.604 | 24.292 | 1.00 | 249.59 |
| 1533 | CB | LYS A | 4 | 5.822 | 17.052 | 16.197 | 1.00 | 225.85 |
| 1534 | CG | LYS A | 4 | 4.918 | 18.220 | 15.853 | 1.00 | 225.85 |
| 1535 | CD | LYS A | 4 | 4.535 | 18.995 | 17.100 | 1.00 | 225.85 |
| 1536 | CE | LYS A | 4 | 3.638 | 20.173 | 16.766 | 1.00 | 225.85 |
| 1537 | N2 | LYS A | 4 | 3.267 | 20.934 | 17.987 | 1.00 | 225.85 |
| 1538 | C | LYS A | 4 | 7.001 | 17.239 | 14.016 | 1.00 | 249.21 |
| 1539 | O | LYS A | 4 | 7.491 | 18.292 | 14.419 | 1.00 | 249.21 |
| 1540 | N | LYS A | 4 | 7.236 | 15.183 | 15.408 | 1.00 | 249.21 |
| 1541 | CA | LYS A | 4 | 6.316 | 16.275 | 14.978 | 1.00 | 249.21 |
| 1542 | N | PRO A | 5 | 7.053 | 16.880 | 12.723 | 1.00 | 94.49 |
| 1543 | CD | PRO A | 5 | 6.773 | 15.535 | 12.187 | 1.00 | 84.99 |
| 1544 | CA | PRO A | 5 | 7.685 | 17.735 | 11.709 | 1.00 | 94.49 |
| 1545 | CB | PRO A | 5 | 8.092 | 16.736 | 10.629 | 1.00 | 84.99 |
| 1546 | CG | PRO A | 5 | 7.010 | 15.716 | 10.691 | 1.00 | 84.99 |
| 1547 | C | PRO A | 5 | 6.772 | 18.825 | 11.164 | 1.00 | 94.49 |
| 1548 | O | PRO A | 5 | 5.557 | 18.721 | 11.227 | 1.00 | 94.49 |
| 1549 | N | LYS A | 6 | 7.358 | 19.877 | 10.617 | 1.00 | 99.70 |
| 1550 | CA | LYS A | 6 | 6.559 | 20.973 | 10.084 | 1.00 | 99.70 |
| 1551 | CB | LYS A | 6 | 6.444 | 22.094 | 11.130 | 1.00 | 128.86 |
| 1552 | CG | LYS A | 6 | 5.540 | 23.242 | 10.711 | 1.00 | 128.86 |
| 1553 | CD | LYS A | 6 | 5.290 | 24.223 | 11.853 | 1.00 | 128.86 |
| 1554 | CE | LYS A | 6 | 4.321 | 25.329 | 11.418 | 1.00 | 128.86 |
| 1555 | NZ | LYS A | 6 | 3.977 | 26.285 | 12.517 | 1.00 | 128.86 |
| 1556 | C | LYS A | 6 | 7.166 | 21.512 | 8.793 | 1.00 | 99.70 |
| 1557 | O | LYS A | 6 | 8.281 | 22.029 | 8.801 | 1.00 | 99.70 |
| 1558 | N | VAL A | 7 | 6.421 | 21.395 | 7.693 | 1.00 | 71.19 |
| 1559 | CA | VAL A | 7 | 6.878 | 21.852 | 6.377 | 1.00 | 71.19 |
| 1560 | CB | VAL A | 7 | 5.955 | 21.392 | 5.243 | 1.00 | 54.73 |
| 1561 | CG1 | VAL A | 7 | 6.584 | 21.739 | 3.900 | 1.00 | 54.73 |
| 1562 | CG2 | VAL A | 7 | 5.687 | 19.922 | 5.350 | 1.00 | 54.73 |
| 1563 | C | VAL A | 7 | 6.947 | 23.354 | 6.221 | 1.00 | 71.19 |
| 1564 | O | VAL A | 7 | 5.924 | 24.023 | 6.282 | 1.00 | 71.19 |
| 1565 | N | SER A | 8 | 8.141 | 23.885 | 5.988 | 1.00 | 76.52 |
| 1566 | CA | SER A | 8 | 8.301 | 25.325 | 5.804 | 1.00 | 76.52 |
| 1567 | CB | SER A | 8 | 9.537 | 25.827 | 6.563 | 1.00 | 232.80 |
| 1568 | OG | SER A | 8 | 10.701 | 25.106 | 6.196 | 1.00 | 232.80 |
| 1569 | C | SER A | 8 | 8.437 | 25.597 | 4.311 | 1.00 | 76.52 |
| 1570 | O | SER A | 8 | 8.665 | 24.679 | 3.534 | 1.00 | 76.52 |
| 1571 | N | LEU A | 9 | 8.274 | 26.851 | 3.914 | 1.00 | 77.48 |
| 1572 | CA | LEU A | 9 | 8.388 | 27.237 | 2.509 | 1.00 | 77.48 |
| 1573 | CB | LEU A | 9 | 7.037 | 27.651 | 1.935 | 1.00 | 70.08 |
| 1574 | CG | LEU A | 9 | 5.879 | 26.663 | 1.868 | 1.00 | 70.08 |
| 1575 | CD1 | LEU A | 9 | 4.901 | 27.105 | 0.816 | 1.00 | 70.08 |
| 1576 | CD2 | LEU A | 9 | 6.399 | 25.305 | 1.517 | 1.00 | 70.08 |
| 1577 | C | LEU A | 9 | 9.321 | 28.417 | 2.334 | 1.00 | 77.48 |
| 1578 | O | LEU A | 9 | 9.506 | 29.212 | 3.257 | 1.00 | 77.48 |
| 1579 | N | ASN A | 10 | 9.896 | 28.544 | 1.140 | 1.00 | 96.41 |
| 1580 | CA | ASN A | 10 | 10.795 | 29.657 | 0.844 | 1.00 | 96.41 |
| 1581 | CB | ASN A | 10 | 12.196 | 29.384 | 1.384 | 1.00 | 121.54 |
| 1582 | CG | ASN A | 10 | 13.074 | 30.616 | 1.338 | 1.00 | 121.54 |
| 1583 | OD1 | ASN A | 10 | 12.819 | 31.598 | 2.041 | 1.00 | 121.54 |
| 1584 | ND2 | ASN A | 10 | 14.106 | 30.581 | 0.496 | 1.00 | 121.54 |
| 1585 | C | ASN A | 10 | 10.868 | 29.920 | −0.654 | 1.00 | 96.41 |
| 1586 | O | ASN A | 10 | 11.396 | 29.110 | −1.412 | 1.00 | 96.41 |
| 1587 | N | PRO A | 11 | 10.325 | 31.064 | −1.105 | 1.00 | 78.36 |
| 1588 | CD | PRO A | 11 | 10.263 | 31.320 | −2.548 | 1.00 | 72.21 |
| 1589 | CA | PRO A | 11 | 9.642 | 32.128 | −0.350 | 1.00 | 78.36 |
| 1590 | CB | PRO A | 11 | 9.130 | 33.049 | −1.455 | 1.00 | 72.21 |
| 1591 | CG | PRO A | 11 | 10.084 | 32.803 | −2.583 | 1.00 | 72.21 |
| 1592 | C | PRO A | 11 | 8.492 | 31.647 | 0.551 | 1.00 | 78.36 |
| 1593 | O | PRO A | 11 | 7.992 | 30.537 | 0.386 | 1.00 | 78.36 |
| 1594 | N | PRO A | 12 | 8.056 | 32.469 | 1.513 | 1.00 | 81.66 |
| 1595 | CD | PRO A | 12 | 8.570 | 33.812 | 1.833 | 1.00 | 122.93 |
| 1596 | CA | PRO A | 12 | 6.968 | 32.110 | 2.430 | 1.00 | 81.66 |
| 1597 | CB | PRO A | 12 | 6.925 | 33.274 | 3.409 | 1.00 | 122.93 |
| 1598 | CG | PRO A | 12 | 8.277 | 33.895 | 3.290 | 1.00 | 122.93 |
| 1599 | C | PRO A | 12 | 5.637 | 31.998 | 1.663 | 1.00 | 81.66 |
| 1600 | O | PRO A | 12 | 4.695 | 31.307 | 2.081 | 1.00 | 81.66 |
| 1601 | N | TRP A | 13 | 5.579 | 32.699 | 0.538 | 1.00 | 66.49 |
| 1602 | CA | TRP A | 13 | 4.388 | 32.725 | −0.293 | 1.00 | 66.49 |
| 1603 | CB | TRP A | 13 | 4.660 | 33.539 | −1.562 | 1.00 | 100.34 |
| 1604 | CG | TRP A | 13 | 5.336 | 34.831 | −1.277 | 1.00 | 100.34 |
| 1605 | CD2 | TRP A | 13 | 5.100 | 35.697 | −0.167 | 1.00 | 100.34 |
| 1606 | CE2 | TRP A | 13 | 6.000 | 36.762 | −0.268 | 1.00 | 100.34 |
| 1607 | CE3 | TRP A | 13 | 4.210 | 35.673 | 0.909 | 1.00 | 100.34 |
| 1608 | CD1 | TRP A | 13 | 6.339 | 35.393 | −1.996 | 1.00 | 100.34 |
| 1609 | NE1 | TRP A | 13 | 6.748 | 36.552 | −1.395 | 1.00 | 100.34 |
| 1610 | CZ2 | TRP A | 13 | 6.046 | 37.795 | 0.664 | 1.00 | 100.34 |
| 1611 | CZ3 | TRP A | 13 | 4.253 | 36.698 | 1.829 | 1.00 | 100.34 |
| 1612 | CH2 | TRP A | 13 | 5.167 | 37.745 | 1.705 | 1.00 | 100.34 |
| 1613 | C | TRP A | 13 | 3.913 | 31.342 | −0.666 | 1.00 | 66.49 |
| 1614 | O | TRP A | 13 | 4.637 | 30.573 | −1.270 | 1.00 | 66.49 |
| 1615 | N | ASN A | 14 | 2.685 | 31.031 | −0.299 | 1.00 | 52.07 |
| 1616 | CA | ASN A | 14 | 2.109 | 29.738 | −0.629 | 1.00 | 52.07 |
| 1617 | CB | ASN A | 14 | 1.508 | 29.082 | 0.626 | 1.00 | 104.36 |
| 1618 | CG | ASN A | 14 | 0.274 | 29.801 | 1.152 | 1.00 | 104.36 |
| 1619 | OD1 | ASN A | 14 | 0.305 | 31.001 | 1.465 | 1.00 | 104.36 |
| 1620 | ND2 | ASN A | 14 | −0.822 | 29.058 | 1.269 | 1.00 | 104.36 |
| 1621 | C | ASN A | 14 | 1.056 | 29.792 | −1.759 | 1.00 | 52.07 |
| 1622 | O | ASN A | 14 | 0.271 | 28.850 | −1.928 | 1.00 | 52.07 |
| 1623 | N | ARG A | 15 | 1.026 | 30.900 | −2.509 | 1.00 | 52.98 |
| 1624 | CA | ARG A | 15 | 0.131 | 31.078 | −3.667 | 1.00 | 52.98 |
| 1625 | CB | ARG A | 15 | −0.942 | 32.109 | −3.415 | 1.00 | 66.97 |
| 1626 | CG | ARG A | 15 | −1.533 | 32.043 | −2.077 | 1.00 | 66.97 |
| 1627 | CD | ARG A | 15 | −2.626 | 33.064 | −2.014 | 1.00 | 66.97 |
| 1628 | NE | ARG A | 15 | −3.768 | 32.699 | −2.837 | 1.00 | 66.97 |
| 1629 | CZ | ARG A | 15 | −4.589 | 33.596 | −3.363 | 1.00 | 66.97 |
| 1630 | NH1 | ARG A | 15 | −4.370 | 34.890 | −3.150 | 1.00 | 66.97 |
| 1631 | NH2 | ARG A | 15 | −5.629 | 33.213 | −4.091 | 1.00 | 66.97 |
| 1632 | C | ARG A | 15 | 1.080 | 31.659 | −4.687 | 1.00 | 52.98 |
| 1633 | O | ARG A | 15 | 1.510 | 32.817 | −4.563 | 1.00 | 52.98 |
| 1634 | N | ILE A | 16 | 1.431 | 30.867 | −5.684 | 1.00 | 61.11 |
| 1635 | CA | ILE A | 16 | 2.362 | 31.362 | −6.667 | 1.00 | 61.11 |
| 1636 | CB | ILE A | 16 | 3.662 | 30.595 | −6.632 | 1.00 | 64.67 |
| 1637 | CG2 | ILE A | 16 | 4.375 | 30.856 | −5.312 | 1.00 | 64.67 |
| 1638 | CG1 | ILE A | 16 | 3.385 | 29.117 | −6.833 | 1.00 | 64.67 |
| 1639 | CD1 | ILE A | 16 | 4.626 | 28.268 | −6.768 | 1.00 | 64.67 |
| 1640 | C | ILE A | 16 | 1.849 | 31.311 | −8.070 | 1.00 | 61.11 |
| 1641 | O | ILE A | 16 | 0.851 | 30.662 | −8.361 | 1.00 | 61.11 |
| 1642 | N | PHE A | 17 | 2.560 | 32.019 | −8.933 | 1.00 | 81.85 |
| 1643 | CA | PHE A | 17 | 2.266 | 32.130 | −10.348 | 1.00 | 81.85 |
| 1644 | CB | PHE A | 17 | 2.902 | 33.411 | −10.856 | 1.00 | 58.17 |
| 1645 | CG | PHE A | 17 | 2.014 | 34.604 | −10.777 | 1.00 | 58.17 |
| 1646 | CD1 | PHE A | 17 | 2.531 | 35.841 | −10.422 | 1.00 | 58.17 |
| 1647 | CD2 | PHE A | 17 | 0.681 | 34.512 | −11.181 | 1.00 | 58.17 |
| 1648 | CE1 | PHE A | 17 | 1.751 | 36.965 | −10.467 | 1.00 | 58.17 |
| 1649 | CE2 | PHE A | 17 | −0.125 | 35.639 | −11.238 | 1.00 | 58.17 |
| 1650 | CZ | PHE A | 17 | 0.415 | 36.876 | −10.885 | 1.00 | 58.17 |
| 1651 | C | PHE A | 17 | 2.851 | 30.940 | −11.110 | 1.00 | 81.85 |
| 1652 | O | PHE A | 17 | 3.749 | 30.259 | −10.621 | 1.00 | 81.85 |
| 1653 | N | LYS A | 18 | 2.353 | 30.699 | −12.314 | 1.00 | 81.40 |
| 1654 | CA | LYS A | 18 | 2.842 | 29.602 | −13.129 | 1.00 | 81.40 |
| 1655 | CB | LYS A | 18 | 1.981 | 29.497 | −14.385 | 1.00 | 133.55 |
| 1656 | CG | LYS A | 18 | 2.281 | 28.313 | −15.277 | 1.00 | 133.55 |
| 1657 | CD | LYS A | 18 | 1.153 | 28.136 | −16.287 | 1.00 | 133.55 |
| 1658 | CE | LYS A | 18 | 1.389 | 26.957 | −17.216 | 1.00 | 133.55 |
| 1659 | NZ | LYS A | 18 | 2.627 | 27.139 | −18.030 | 1.00 | 133.55 |
| 1660 | C | LYS A | 18 | 4.305 | 29.838 | −13.515 | 1.00 | 81.40 |
| 1661 | O | LYS A | 18 | 4.683 | 30.921 | −13.972 | 1.00 | 81.40 |
| 1662 | N | GLY A | 19 | 5.141 | 28.834 | −13.313 | 1.00 | 92.32 |
| 1663 | CA | GLY A | 19 | 6.524 | 28.975 | −13.702 | 1.00 | 92.32 |
| 1664 | C | GLY A | 19 | 7.492 | 29.428 | −12.643 | 1.00 | 92.32 |
| 1665 | O | GLY A | 19 | 8.697 | 29.398 | −12.866 | 1.00 | 92.32 |
| 1666 | N | GLU A | 20 | 6.996 | 29.853 | −11.491 | 1.00 | 67.13 |
| 1667 | CA | GLU A | 20 | 7.896 | 30.300 | −10.422 | 1.00 | 67.13 |
| 1668 | CB | GLU A | 20 | 7.153 | 31.239 | −9.477 | 1.00 | 115.51 |
| 1669 | CG | GLU A | 20 | 6.439 | 32.361 | −10.221 | 1.00 | 115.51 |
| 1670 | CD | GLU A | 20 | 5.794 | 33.361 | −9.300 | 1.00 | 115.51 |
| 1671 | OE1 | GLU A | 20 | 4.991 | 32.949 | −8.432 | 1.00 | 115.51 |
| 1672 | OE2 | GLU A | 20 | 6.091 | 34.561 | −9.454 | 1.00 | 115.51 |
| 1673 | C | GLU A | 20 | 8.469 | 29.094 | −9.652 | 1.00 | 67.13 |
| 1674 | O | GLU A | 20 | 8.035 | 27.953 | −9.861 | 1.00 | 67.13 |
| 1675 | N | ASN A | 21 | 9.456 | 29.329 | −8.788 | 1.00 | 81.05 |
| 1676 | CA | ASN A | 21 | 10.059 | 28.225 | −8.040 | 1.00 | 81.05 |
| 1677 | CB | ASN A | 21 | 11.562 | 28.078 | −8.328 | 1.00 | 110.52 |
| 1678 | CG | ASN A | 21 | 11.923 | 28.283 | −9.788 | 1.00 | 110.52 |
| 1679 | OD1 | ASN A | 21 | 11.250 | 27.808 | −10.699 | 1.00 | 110.52 |
| 1680 | ND2 | ASN A | 21 | 13.025 | 28.989 | −9.995 | 1.00 | 110.52 |
| 1681 | C | ASN A | 21 | 9.915 | 28.409 | −6.547 | 1.00 | 81.05 |
| 1682 | O | ASN A | 21 | 10.054 | 29.521 | −6.035 | 1.00 | 81.05 |
| 1683 | N | VAL A | 22 | 9.681 | 27.306 | −5.848 | 1.00 | 79.17 |
| 1684 | CA | VAL A | 22 | 9.525 | 27.341 | −4.404 | 1.00 | 79.17 |
| 1685 | CB | VAL A | 22 | 8.057 | 27.304 | −4.012 | 1.00 | 85.34 |
| 1686 | CG1 | VAL A | 22 | 7.431 | 26.001 | −4.486 | 1.00 | 85.34 |
| 1687 | CG2 | VAL A | 22 | 7.925 | 27.449 | −2.510 | 1.00 | 85.34 |
| 1688 | C | VAL A | 22 | 10.194 | 26.117 | −3.815 | 1.00 | 79.17 |
| 1689 | O | VAL A | 22 | 10.247 | 25.070 | −4.469 | 1.00 | 79.17 |
| 1690 | N | THR A | 23 | 10.676 | 26.240 | −2.579 | 1.00 | 92.04 |
| 1691 | CA | THR A | 23 | 11.367 | 25.145 | −1.908 | 1.00 | 92.04 |
| 1692 | CB | THR A | 23 | 12.775 | 25.585 | −1.556 | 1.00 | 153.40 |
| 1693 | OG1 | THR A | 23 | 13.414 | 26.089 | −2.736 | 1.00 | 153.40 |
| 1694 | CG2 | THR A | 23 | 13.567 | 24.428 | −0.993 | 1.00 | 153.40 |
| 1695 | C | THR A | 23 | 10.667 | 24.698 | −0.634 | 1.00 | 92.04 |
| 1696 | O | THR A | 23 | 10.364 | 25.525 | 0.212 | 1.00 | 92.04 |
| 1697 | N | LEU A | 24 | 10.403 | 23.404 | −0.485 | 1.00 | 64.92 |
| 1698 | CA | LEU A | 24 | 9.742 | 22.945 | 0.730 | 1.00 | 64.92 |
| 1699 | CB | LEU A | 24 | 8.564 | 22.015 | 0.427 | 1.00 | 83.07 |
| 1700 | CG | LEU A | 24 | 7.676 | 22.301 | −0.774 | 1.00 | 83.07 |
| 1701 | CD1 | LEU A | 24 | 6.400 | 21.482 | −0.676 | 1.00 | 83.07 |
| 1702 | CD2 | LEU A | 24 | 7.348 | 23.745 | −0.837 | 1.00 | 83.07 |
| 1703 | C | LEU A | 24 | 10.701 | 22.206 | 1.657 | 1.00 | 64.92 |
| 1704 | O | LEU A | 24 | 11.034 | 21.049 | 1.433 | 1.00 | 64.92 |
| 1705 | N | THR A | 25 | 11.125 | 22.863 | 2.725 | 1.00 | 60.46 |
| 1706 | CA | THR A | 25 | 12.026 | 22.227 | 3.665 | 1.00 | 60.46 |
| 1707 | CB | THR A | 25 | 12.890 | 23.286 | 4.309 | 1.00 | 96.68 |
| 1708 | OG1 | THR A | 25 | 13.523 | 24.040 | 3.273 | 1.00 | 96.68 |
| 1709 | CG2 | THR A | 25 | 13.943 | 22.654 | 5.175 | 1.00 | 96.68 |
| 1710 | C | THR A | 25 | 11.264 | 21.446 | 4.746 | 1.00 | 60.46 |
| 1711 | O | THR A | 25 | 10.270 | 21.923 | 5.293 | 1.00 | 60.46 |
| 1712 | N | CYS A | 26 | 11.717 | 20.239 | 5.048 | 1.00 | 126.10 |
| 1713 | CA | CYS A | 26 | 11.060 | 19.464 | 6.081 | 1.00 | 126.10 |
| 1714 | C | CYS A | 26 | 11.617 | 19.884 | 7.421 | 1.00 | 126.10 |
| 1715 | O | CYS A | 26 | 12.813 | 20.108 | 7.566 | 1.00 | 126.10 |
| 1716 | CB | CYS A | 26 | 11.293 | 17.971 | 5.888 | 1.00 | 188.87 |
| 1717 | SG | CYS A | 26 | 10.283 | 16.954 | 7.005 | 1.00 | 188.87 |
| 1718 | N | ASN A | 27 | 10.727 | 19.999 | 8.393 | 1.00 | 248.12 |
| 1719 | CA | ASN A | 27 | 11.065 | 20.379 | 9.747 | 1.00 | 248.12 |
| 1720 | CB | ASN A | 27 | 10.474 | 19.354 | 10.685 | 1.00 | 249.30 |
| 1721 | CG | ASN A | 27 | 10.331 | 19.883 | 12.046 | 1.00 | 249.30 |
| 1722 | OD1 | ASN A | 27 | 9.999 | 21.050 | 12.192 | 1.00 | 249.30 |
| 1723 | ND2 | ASN A | 27 | 10.582 | 19.060 | 13.069 | 1.00 | 249.30 |
| 1724 | C | ASN A | 27 | 12.549 | 20.546 | 10.040 | 1.00 | 248.12 |
| 1725 | O | ASN A | 27 | 13.220 | 19.591 | 10.431 | 1.00 | 248.12 |
| 1726 | N | GLY A | 28 | 13.058 | 21.754 | 9.840 | 1.00 | 150.98 |
| 1727 | CA | GLY A | 28 | 14.469 | 22.013 | 10.073 | 1.00 | 150.98 |
| 1728 | C | GLY A | 28 | 14.771 | 23.413 | 9.596 | 1.00 | 150.98 |
| 1729 | O | GLY A | 28 | 14.541 | 23.731 | 8.435 | 1.00 | 150.98 |
| 1730 | N | ASN A | 29 | 15.288 | 24.258 | 10.480 | 1.00 | 168.28 |
| 1731 | CA | ASN A | 29 | 15.576 | 25.638 | 10.111 | 1.00 | 168.28 |
| 1732 | CB | ASN A | 29 | 15.714 | 26.494 | 11.374 | 1.00 | 185.34 |
| 1733 | CG | ASN A | 29 | 15.723 | 27.979 | 11.072 | 1.00 | 185.34 |
| 1734 | OD1 | ASN A | 29 | 15.387 | 28.400 | 9.966 | 1.00 | 185.34 |
| 1735 | ND2 | ASN A | 29 | 16.097 | 28.782 | 12.059 | 1.00 | 185.34 |
| 1736 | C | ASN A | 29 | 16.799 | 25.839 | 9.208 | 1.00 | 168.28 |
| 1737 | O | ASN A | 29 | 16.704 | 26.492 | 8.165 | 1.00 | 168.28 |
| 1738 | N | ASN A | 30 | 17.943 | 25.279 | 9.594 | 1.00 | 244.43 |
| 1739 | CA | ASN A | 30 | −19.151 | 25.453 | 8.797 | 1.00 | 244.43 |
| 1740 | CB | ASN A | 30 | 20.131 | 26.363 | 9.543 | 1.00 | 249.25 |
| 1741 | CG | ASN A | 30 | 19.592 | 27.765 | 9.735 | 1.00 | 249.25 |
| 1742 | OD1 | ASN A | 30 | 19.601 | 28.297 | 10.843 | 1.00 | 249.25 |
| 1743 | ND2 | ASN A | 30 | 19.122 | 28.372 | 8.654 | 1.00 | 249.25 |
| 1744 | C | ASN A | 30 | 19.863 | 24.172 | 8.412 | 1.00 | 244.43 |
| 1745 | O | ASN A | 30 | 19.859 | 23.770 | 7.252 | 1.00 | 244.43 |
| 1746 | N | PHE A | 31 | 20.478 | 23.527 | 9.386 | 1.00 | 249.41 |
| 1747 | CA | PHE A | 31 | 21.210 | 22.326 | 9.077 | 1.00 | 249.41 |
| 1748 | CB | PHE A | 31 | 22.639 | 22.474 | 9.586 | 1.00 | 249.46 |
| 1749 | CG | PHE A | 31 | 23.362 | 23.675 | 9.073 | 1.00 | 249.46 |
| 1750 | CD1 | PHE A | 31 | 23.138 | 24.925 | 9.634 | 1.00 | 249.46 |
| 1751 | CD2 | PHE A | 31 | 24.250 | 23.563 | 8.008 | 1.00 | 249.46 |
| 1752 | CE1 | PHE A | 31 | 23.798 | 26.050 | 9.152 | 1.00 | 249.46 |
| 1753 | CE2 | PHE A | 31 | 24.917 | 24.682 | 7.514 | 1.00 | 249.46 |
| 1754 | CZ | PHE A | 31 | 24.682 | 25.931 | 8.083 | 1.00 | 249.46 |
| 1755 | C | PHE A | 31 | 20.559 | 21.049 | 9.617 | 1.00 | 249.41 |
| 1756 | O | PHE A | 31 | 20.226 | 20.949 | 10.807 | 1.00 | 249.41 |
| 1757 | N | PHE A | 32 | 20.393 | 20.077 | 8.715 | 1.00 | 249.47 |
| 1758 | CA | PHE A | 32 | 19.790 | 18.777 | 9.021 | 1.00 | 249.47 |
| 1759 | CB | PHE A | 32 | 18.496 | 18.614 | 8.228 | 1.00 | 246.45 |
| 1760 | CG | PHE A | 32 | 17.642 | 17.487 | 8.707 | 1.00 | 246.45 |
| 1761 | CD1 | PHE A | 32 | 17.048 | 17.548 | 9.963 | 1.00 | 246.45 |
| 1762 | CD2 | PHE A | 32 | 17.442 | 16.359 | 7.921 | 1.00 | 246.45 |
| 1763 | CE1 | PHE A | 32 | 16.272 | 16.499 | 10.437 | 1.00 | 246.45 |
| 1764 | CE2 | PHE A | 32 | 16.665 | 15.302 | 8.387 | 1.00 | 246.45 |
| 1765 | CZ | PHE A | 32 | 16.077 | 15.378 | 9.652 | 1.00 | 246.45 |
| 1766 | C | PHE A | 32 | 20.742 | 17.630 | 8.674 | 1.00 | 249.47 |
| 1767 | O | PHE A | 32 | 21.773 | 17.852 | 8.051 | 1.00 | 249.47 |
| 1768 | N | GLU A | 33 | 20.392 | 16.403 | 9.058 | 1.00 | 249.57 |
| 1769 | CA | GLU A | 33 | 21.260 | 15.270 | 8.763 | 1.00 | 249.57 |
| 1770 | CB | GLU A | 33 | 21.850 | 14.696 | 10.034 | 1.00 | 249.41 |
| 1771 | CG | GLU A | 33 | 22.893 | 13.655 | 9.727 | 1.00 | 249.41 |
| 1772 | CD | GLU A | 33 | 24.096 | 14.255 | 9.017 | 1.00 | 249.41 |
| 1773 | OE1 | GLU A | 33 | 24.471 | 15.388 | 9.373 | 1.00 | 249.41 |
| 1774 | OE2 | GLU A | 33 | 24.695 | 13.581 | 8.140 | 1.00 | 249.41 |
| 1775 | C | GLU A | 33 | 20.671 | 14.104 | 7.992 | 1.00 | 249.57 |
| 1776 | O | GLU A | 33 | 21.232 | 13.684 | 6.982 | 1.00 | 249.57 |
| 1777 | N | VAL A | 34 | 19.566 | 13.554 | 8.485 | 1.00 | 216.78 |
| 1778 | CA | VAL A | 34 | 18.961 | 12.405 | 7.832 | 1.00 | 216.78 |
| 1779 | CB | VAL A | 34 | 17.623 | 12.017 | 8.499 | 1.00 | 196.07 |
| 1780 | CG1 | VAL A | 34 | 17.008 | 10.816 | 7.801 | 1.00 | 196.07 |
| 1781 | CG2 | VAL A | 34 | 17.864 | 11.683 | 9.958 | 1.00 | 196.07 |
| 1782 | C | VAL A | 34 | 18.754 | 12.609 | 6.338 | 1.00 | 216.78 |
| 1783 | O | VAL A | 34 | 18.550 | 13.729 | 5.860 | 1.00 | 216.78 |
| 1784 | N | SER A | 35 | 18.845 | 11.506 | 5.608 | 1.00 | 172.95 |
| 1785 | CA | SER A | 35 | 18.669 | 11.506 | 4.170 | 1.00 | 172.95 |
| 1786 | CB | SER A | 35 | 19.837 | 10.789 | 3.489 | 1.00 | 249.26 |
| 1787 | OG | SER A | 35 | 19.822 | 9.399 | 3.775 | 1.00 | 249.26 |
| 1788 | C | SEH A | 35 | 17.368 | 10.770 | 3.873 | 1.00 | 172.95 |
| 1789 | O | SER A | 35 | 16.978 | 10.632 | 2.715 | 1.00 | 172.95 |
| 1790 | N | SER A | 36 | 16.706 | 10.290 | 4.926 | 1.00 | 142.42 |
| 1791 | CA | SER A | 36 | 15.437 | 9.579 | 4.773 | 1.00 | 142.42 |
| 1792 | CB | SER A | 36 | 15.404 | 8.320 | 5.643 | 1.00 | 183.21 |
| 1793 | OG | SER A | 36 | 15.320 | 8.643 | 7.020 | 1.00 | 183.21 |
| 1794 | C | SER A | 36 | 14.288 | 10.498 | 5.168 | 1.00 | 142.42 |
| 1795 | O | SER A | 36 | 13.906 | 10.585 | 6.337 | 1.00 | 142.42 |
| 1796 | N | THR A | 37 | 13.749 | 11.189 | 4.171 | 1.00 | 91.48 |
| 1797 | CA | THR A | 37 | 12.645 | 12.117 | 4.370 | 1.00 | 91.48 |
| 1798 | CB | THR A | 37 | 13.088 | 13.579 | 4.085 | 1.00 | 110.07 |
| 1799 | OG1 | THR A | 37 | 14.193 | 13.929 | 4.928 | 1.00 | 110.07 |
| 1800 | CG2 | THR A | 37 | 11.960 | 14.535 | 4.352 | 1.00 | 110.07 |
| 1801 | C | THR A | 37 | 11.582 | 11.689 | 3.366 | 1.00 | 91.48 |
| 1802 | O | THR A | 37 | 11.902 | 11.294 | 2.244 | 1.00 | 91.48 |
| 1803 | N | LYS A | 38 | 10.321 | 11.748 | 3.769 | 1.00 | 121.21 |
| 1804 | CA | LYS A | 38 | 9.233 | 11.345 | 2.886 | 1.00 | 121.21 |
| 1805 | CB | LYS A | 38 | 8.339 | 10.344 | 3.600 | 1.00 | 152.68 |
| 1806 | CG | LYS A | 38 | 9.088 | 9.131 | 4.112 | 1.00 | 152.68 |
| 1807 | CD | LYS A | 38 | 8.151 | 8.168 | 4.824 | 1.00 | 152.68 |
| 1808 | CE | LYS A | 38 | 8.877 | 6.909 | 5.245 | 1.00 | 152.68 |
| 1809 | NZ | LYS A | 38 | 7.952 | 5.951 | 5.893 | 1.00 | 152.68 |
| 1810 | C | LYS A | 38 | 8.389 | 12.529 | 2.442 | 1.00 | 121.21 |
| 1811 | O | LYS A | 38 | 8.140 | 13.440 | 3.226 | 1.00 | 121.21 |
| 1812 | N | TRP A | 39 | 7.954 | 12.517 | 1.185 | 1.00 | 102.82 |
| 1813 | CA | TRP A | 39 | 7.119 | 13.592 | 0.656 | 1.00 | 102.82 |
| 1814 | CB | TRP A | 39 | 7.861 | 14.401 | −0.401 | 1.00 | 80.70 |
| 1815 | CG | TRP A | 39 | 9.037 | 15.167 | 0.113 | 1.00 | 80.70 |
| 1816 | CD2 | TRP A | 39 | 9.022 | 16.295 | 0.994 | 1.00 | 80.70 |
| 1817 | CE2 | TRP A | 39 | 10.366 | 16.677 | 1.201 | 1.00 | 80.70 |
| 1818 | CE3 | TRP A | 39 | 8.002 | 17.015 | 1.634 | 1.00 | 80.70 |
| 1819 | CD1 | TRP A | 39 | 10.351 | 14.922 | −0.168 | 1.00 | 80.70 |
| 1820 | NE1 | TRP A | 39 | 11.154 | 15.826 | 0.484 | 1.00 | 80.70 |
| 1821 | CZ2 | TRP A | 39 | 10.717 | 17.745 | 2.011 | 1.00 | 80.70 |
| 1822 | CZ3 | TRP A | 39 | 8.355 | 18.082 | 2.443 | 1.00 | 80.70 |
| 1823 | CH2 | TRP A | 39 | 9.703 | 18.438 | 2.623 | 1.00 | 80.70 |
| 1824 | C | TRP A | 39 | 5.875 | 13.008 | 0.026 | 1.00 | 102.82 |
| 1825 | O | TRP A | 39 | 5.956 | 12.079 | −0.765 | 1.00 | 102.82 |
| 1826 | N | PHE A | 40 | 4.724 | 13.562 | 0.368 | 1.00 | 102.87 |
| 1827 | CA | PHE A | 40 | 3.489 | 13.049 | −0.175 | 1.00 | 102.87 |
| 1828 | CB | PHE A | 40 | 2.633 | 12.434 | 0.936 | 1.00 | 104.88 |
| 1829 | CG | PHE A | 40 | 3.319 | 11.346 | 1.706 | 1.00 | 104.88 |
| 1830 | CD1 | PHE A | 40 | 4.222 | 11.655 | 2.715 | 1.00 | 104.88 |
| 1831 | CD2 | PHE A | 40 | 3.050 | 10.011 | 1.438 | 1.00 | 104.88 |
| 1832 | CE1 | PHE A | 40 | 4.847 | 10.652 | 3.448 | 1.00 | 104.88 |
| 1833 | CE2 | PHE A | 40 | 3.672 | 8.999 | 2.167 | 1.00 | 104.88 |
| 1834 | CZ | PHE A | 40 | 4.570 | 9.321 | 3.174 | 1.00 | 104.88 |
| 1835 | C | PHE A | 40 | 2.676 | 14.104 | −0.898 | 1.00 | 102.87 |
| 1836 | O | PHE A | 40 | 1.808 | 14.741 | −0.302 | 1.00 | 102.87 |
| 1837 | N | HIS A | 41 | 2.952 | 14.287 | −2.184 | 1.00 | 73.61 |
| 1838 | CA | HIS A | 41 | 2.205 | 15.252 | −2.984 | 1.00 | 73.61 |
| 1839 | CB | HIS A | 41 | 2.986 | 15.552 | −4.254 | 1.00 | 81.93 |
| 1840 | CG | HIS A | 41 | 2.304 | 16.514 | −5.162 | 1.00 | 81.93 |
| 1841 | CD2 | HIS A | 41 | 2.173 | 16.521 | −6.507 | 1.00 | 81.93 |
| 1842 | ND1 | HIS A | 41 | 1.661 | 17.645 | −4.706 | 1.00 | 81.93 |
| 1843 | CE1 | HIS A | 41 | 1.158 | 18.309 | −5.731 | 1.00 | 81.93 |
| 1844 | NE2 | HIS A | 41 | 1.455 | 17.648 | −6.837 | 1.00 | 81.93 |
| 1845 | C | HIS A | 41 | 0.811 | 14.687 | −3.318 | 1.00 | 73.61 |
| 1846 | O | HIS A | 41 | 0.690 | 13.733 | −4.088 | 1.00 | 73.61 |
| 1847 | N | ASN A | 42 | −0.234 | 15.280 | −2.740 | 1.00 | 96.75 |
| 1848 | CA | ASN A | 42 | −1.617 | 14.822 | −2.940 | 1.00 | 96.75 |
| 1849 | CB | ASN A | 42 | −2.017 | 14.809 | −4.435 | 1.00 | 98.09 |
| 1850 | CG | ASN A | 42 | −2.244 | 16.205 | −5.004 | 1.00 | 98.09 |
| 1851 | OD1 | ASN A | 42 | −1.466 | 17.108 | −4.726 | 1.00 | 98.09 |
| 1852 | ND2 | ASN A | 42 | −3.284 | 16.385 | −5.814 | 1.00 | 98.09 |
| 1853 | C | ASN A | 42 | −1.771 | 13.413 | −2.374 | 1.00 | 96.75 |
| 1854 | O | ASN A | 42 | −2.625 | 12.652 | −2.826 | 1.00 | 96.75 |
| 1855 | N | GLY A | 43 | −0.948 | 13.068 | −1.386 | 1.00 | 89.87 |
| 1356 | CA | GLY A | 43 | −1.019 | 11.739 | −0.789 | 1.00 | 89.87 |
| 1857 | C | GLY A | 43 | −0.054 | 10.730 | −1.410 | 1.00 | 89.87 |
| 1858 | O | GLY A | 43 | 0.542 | 9.901 | −0.714 | 1.00 | 89.87 |
| 1859 | N | SER A | 44 | 0.097 | 10.798 | −2.728 | 1.00 | 129.29 |
| 1860 | CA | SER A | 44 | 0.990 | 9.904 | −3.449 | 1.00 | 129.29 |
| 1861 | CB | SER A | 44 | 0.833 | 10.113 | −4.960 | 1.00 | 173.89 |
| 1862 | OG | SER A | 44 | −0.521 | 10.004 | −5.358 | 1.00 | 173.89 |
| 1863 | C | SER A | 44 | 2.436 | 10.182 | −3.043 | 1.00 | 129.29 |
| 1864 | O | SER A | 44 | 2.890 | 11.322 | −3.095 | 1.00 | 129.29 |
| 1865 | N | LEU A | 45 | 3.159 | 9.142 | −2.639 | 1.00 | 128.43 |
| 1866 | CA | LEU A | 45 | 4.559 | 9.291 | −2.239 | 1.00 | 128.43 |
| 1867 | CB | LEU A | 45 | 5.149 | 7.925 | −1.874 | 1.00 | 210.08 |
| 1868 | CG | LEU A | 45 | 6.602 | 7.911 | −1.397 | 1.00 | 210.08 |
| 1869 | CD1 | LEU A | 45 | 6.768 | 8.881 | −0.237 | 1.00 | 210.08 |
| 1870 | CD2 | LEU A | 45 | 6.995 | 6.495 | −0.980 | 1.00 | 210.08 |
| 1871 | C | LEU A | 45 | 5.379 | 9.921 | −3.365 | 1.00 | 128.43 |
| 1872 | O | LEU A | 45 | 5.129 | 9.671 | −4.540 | 1.00 | 128.43 |
| 1873 | N | SER A | 46 | 6.354 | 10.749 | −3.007 | 1.00 | 150.05 |
| 1874 | CA | SER A | 46 | 7.200 | 11.403 | −4.006 | 1.00 | 150.05 |
| 1875 | CB | SER A | 46 | 7.500 | 12.846 | −3.588 | 1.00 | 129.32 |
| 1876 | OG | SER A | 46 | 8.251 | 13.516 | −4.586 | 1.00 | 129.32 |
| 1877 | C | SER A | 46 | 8.499 | 10.623 | −4.127 | 1.00 | 150.05 |
| 1878 | O | SER A | 46 | 8.801 | 9.796 | −3.275 | 1.00 | 150.05 |
| 1879 | N | GLU A | 47 | 9.274 | 10.881 | −5.177 | 1.00 | 207.01 |
| 1880 | CA | GLU A | 47 | 10.534 | 10.168 | −5.357 | 1.00 | 207.01 |
| 1881 | CB | GLU A | 47 | 10.798 | 9.896 | −6.851 | 1.00 | 249.57 |
| 1882 | CG | GLU A | 47 | 9.574 | 9.479 | −7.672 | 1.00 | 249.57 |
| 1883 | CD | GLU A | 47 | 9.801 | 9.602 | −9.185 | 1.00 | 249.57 |
| 1884 | OE1 | GLU A | 47 | 9.668 | 10.722 | −9.729 | 1.00 | 249.57 |
| 1885 | OE2 | GLU A | 47 | 10.133 | 8.577 | −9.821 | 1.00 | 249.57 |
| 1886 | C | GLU A | 47 | 11.743 | 10.894 | −4.739 | 1.00 | 207.01 |
| 1887 | O | GLU A | 47 | 12.856 | 10.373 | −4.796 | 1.00 | 207.01 |
| 1888 | N | GLU A | 48 | 11.556 | 12.084 | −4.163 | 1.00 | 127.05 |
| 1889 | CA | GLU A | 48 | 12.703 | 12.760 | −3.542 | 1.00 | 127.05 |
| 1890 | CB | GLU A | 48 | 12.524 | 14.292 | −3.489 | 1.00 | 182.29 |
| 1891 | CG | GLU A | 48 | 13.615 | 15.057 | −2.682 | 1.00 | 182.29 |
| 1892 | CD | GLU A | 48 | 15.017 | 15.011 | −3.296 | 1.00 | 182.29 |
| 1893 | OE1 | GLU A | 48 | 15.226 | 15.631 | −4.359 | 1.00 | 182.29 |
| 1894 | OE2 | GLU A | 48 | 15.914 | 14.360 | −2.713 | 1.00 | 182.29 |
| 1895 | C | GLU A | 48 | 12.882 | 12.208 | −2.126 | 1.00 | 127.05 |
| 1896 | O | GLU A | 48 | 11.938 | 11.681 | −1.531 | 1.00 | 127.05 |
| 1897 | N | THR A | 49 | 14.099 | 12.305 | −1.600 | 1.00 | 86.20 |
| 1898 | CA | THR A | 49 | 14.385 | 11.817 | −0.258 | 1.00 | 86.20 |
| 1899 | CB | THR A | 49 | 15.263 | 10.549 | −0.313 | 1.00 | 133.36 |
| 1900 | OG1 | THR A | 49 | 16.473 | 10.832 | −1.027 | 1.00 | 133.36 |
| 1901 | CG2 | THR A | 49 | 14.513 | 9.419 | −1.021 | 1.00 | 133.36 |
| 1902 | C | THR A | 49 | 15.074 | 12.903 | 0.583 | 1.00 | 86.20 |
| 1903 | O | THR A | 49 | 14.950 | 12.938 | 1.810 | 1.00 | 86.20 |
| 1904 | N | ASN A | 50 | 15.787 | 13.801 | −0.085 | 1.00 | 156.26 |
| 1905 | CA | ASN A | 50 | 16.465 | 14.888 | 0.610 | 1.00 | 156.26 |
| 1906 | CB | ASN A | 50 | 17.158 | 15.810 | −0.406 | 1.00 | 185.93 |
| 1907 | CG | ASN A | 50 | 18.159 | 16.752 | 0.245 | 1.00 | 185.93 |
| 1908 | OD1 | ASN A | 50 | 18.105 | 16.970 | 1.452 | 1.00 | 185.93 |
| 1909 | ND2 | ASN A | 50 | 19.062 | 17.323 | −0.549 | 1.00 | 185.93 |
| 1910 | C | ASN A | 50 | 15.393 | 15.656 | 1.382 | 1.00 | 156.26 |
| 1911 | O | ASN A | 50 | 14.238 | 15.689 | 0.976 | 1.00 | 156.26 |
| 1912 | N | SER A | 51 | 15.765 | 16.264 | 2.499 | 1.00 | 124.65 |
| 1913 | CA | SER A | 51 | 14.804 | 17.019 | 3.296 | 1.00 | 124.65 |
| 1914 | CB | SER A | 51 | 15.434 | 17.440 | 4.628 | 1.00 | 124.86 |
| 1915 | OG | SER A | 51 | 16.427 | 18.441 | 4.450 | 1.00 | 124.86 |
| 1916 | C | SER A | 51 | 14.281 | 18.263 | 2.569 | 1.00 | 124.65 |
| 1917 | O | SER A | 51 | 13.257 | 18.823 | 2.959 | 1.00 | 124.65 |
| 1918 | N | SER A | 52 | 14.979 | 18.704 | 1.525 | 1.00 | 90.69 |
| 1919 | CA | SER A | 52 | 14.553 | 19.884 | 0.780 | 1.00 | 90.69 |
| 1920 | CB | SER A | 52 | 15.708 | 20.872 | 0.631 | 1.00 | 131.83 |
| 1921 | OG | SER A | 52 | 16.109 | 21.377 | 1.894 | 1.00 | 131.83 |
| 1922 | C | SER A | 52 | 14.038 | 19.478 | −0.584 | 1.00 | 90.69 |
| 1923 | O | SER A | 52 | 14.803 | 19.073 | −1.449 | 1.00 | 90.69 |
| 1924 | N | LEU A | 53 | 12.727 | 19.584 | −0.756 | 1.00 | 92.73 |
| 1925 | CA | LEU A | 53 | 12.057 | 19.239 | −2.005 | 1.00 | 92.73 |
| 1926 | CB | LEU A | 53 | 10.720 | 18.547 | −1.710 | 1.00 | 96.57 |
| 1927 | CG | LEU A | 53 | 9.633 | 18.561 | −2.788 | 1.00 | 96.57 |
| 1928 | CD1 | LEU A | 53 | 10.226 | 18.224 | −4.145 | 1.00 | 96.57 |
| 1929 | CD2 | LEU A | 53 | 8.536 | 17.571 | −2.396 | 1.00 | 96.57 |
| 1930 | C | LEU A | 53 | 11.814 | 20.486 | −2.847 | 1.00 | 92.73 |
| 1931 | O | LEU A | 53 | 10.874 | 21.231 | −2.601 | 1.00 | 92.73 |
| 1932 | N | ASN A | 54 | 12.660 | 20.710 | −3.846 | 1.00 | 74.24 |
| 1933 | CA | ASN A | 54 | 12.508 | 21.879 | −4.708 | 1.00 | 74.24 |
| 1934 | CB | ASN A | 54 | 13.819 | 22.180 | −5.442 | 1.00 | 143.36 |
| 1935 | CG | ASN A | 54 | 14.883 | 22.734 | −4.526 | 1.00 | 143.36 |
| 1936 | OD1 | ASN A | 54 | 14.670 | 23.738 | −3.853 | 1.00 | 143.36 |
| 1937 | ND2 | ASN A | 54 | 16.040 | 22.086 | −4.497 | 1.00 | 143.36 |
| 1938 | C | ASN A | 54 | 11.390 | 21.731 | −5.727 | 1.00 | 74.24 |
| 1939 | O | ASN A | 54 | 10.937 | 20.633 | −6.038 | 1.00 | 74.24 |
| 1940 | N | ILE A | 55 | 10.936 | 22.868 | −6.233 | 1.00 | 93.23 |
| 1941 | CA | ILE A | 55 | 9.898 | 22.911 | −7.249 | 1.00 | 93.23 |
| 1942 | CB | ILE A | 55 | 8.542 | 23.323 | −6.659 | 1.00 | 75.25 |
| 1943 | CG2 | ILE A | 55 | 7.629 | 23.783 | −7.751 | 1.00 | 75.25 |
| 1944 | CG1 | ILE A | 55 | 7.932 | 22.135 | −5.918 | 1.00 | 75.25 |
| 1945 | CD1 | ILE A | 55 | 6.605 | 22.397 | −5.286 | 1.00 | 75.25 |
| 1946 | C | ILE A | 55 | 10.359 | 23.951 | −8.241 | 1.00 | 93.23 |
| 1947 | O | ILE A | 55 | 10.593 | 25.100 | −7.866 | 1.00 | 93.23 |
| 1948 | N | VAL A | 56 | 10.528 | 23.543 | −9.491 | 1.00 | 114.64 |
| 1949 | CA | VAL A | 56 | 10.977 | 24.469 | −10.515 | 1.00 | 114.64 |
| 1950 | CB | VAL A | 56 | 12.025 | 23.820 | −11.419 | 1.00 | 202.78 |
| 1951 | CG1 | VAL A | 56 | 12.782 | 24.892 | −12.183 | 1.00 | 202.78 |
| 1952 | CG2 | VAL A | 56 | 12.983 | 22.997 | −10.579 | 1.00 | 202.78 |
| 1953 | C | VAL A | 56 | 9.771 | 24.909 | −11.333 | 1.00 | 114.64 |
| 1954 | O | VAL A | 56 | 8.649 | 24.730 | −10.883 | 1.00 | 114.64 |
| 1955 | N | ASN A | 57 | 9.993 | 25.480 | −12.516 | 1.00 | 86.89 |
| 1956 | CA | ASN A | 57 | 8.902 | 25.961 | −13.366 | 1.00 | 86.89 |
| 1957 | CB | ASN A | 57 | 9.187 | 25.646 | −14.832 | 1.00 | 171.09 |
| 1958 | CG | ASN A | 57 | 10.333 | 26.468 | −15.379 | 1.00 | 171.09 |
| 1959 | OD1 | ASN A | 57 | 10.332 | 27.695 | −15.277 | 1.00 | 171.09 |
| 1960 | ND2 | ASN A | 57 | 11.318 | 25.799 | −15.962 | 1.00 | 171.09 |
| 1961 | C | ASN A | 57 | 7.549 | 25.397 | −12.962 | 1.00 | 86.89 |
| 1962 | O | ASN A | 57 | 7.112 | 24.377 | −13.473 | 1.00 | 86.89 |
| 1963 | N | ALA A | 58 | 6.893 | 26.087 | −12.036 | 1.00 | 98.74 |
| 1964 | CA | ALA A | 58 | 5.610 | 25.665 | −11.500 | 1.00 | 98.74 |
| 1965 | CB | ALA A | 58 | 5.094 | 26.705 | −10.525 | 1.00 | 108.16 |
| 1966 | C | ALA A | 58 | 4.557 | 25.376 | −12.548 | 1.00 | 98.74 |
| 1967 | O | ALA A | 58 | 4.185 | 26.242 | −13.327 | 1.00 | 98.74 |
| 1968 | N | LYS A | 59 | 4.082 | 24.140 | −12.560 | 1.00 | 74.98 |
| 1969 | CA | LYS A | 59 | 3.039 | 23.725 | −13.482 | 1.00 | 74.98 |
| 1970 | CB | LYS A | 59 | 3.424 | 22.395 | −14.146 | 1.00 | 178.83 |
| 1971 | CG | LYS A | 59 | 4.740 | 22.455 | −14.920 | 1.00 | 178.83 |
| 1972 | CD | LYS A | 59 | 5.158 | 21.095 | −15.463 | 1.00 | 178.83 |
| 1973 | CE | LYS A | 59 | 6.483 | 21.185 | −16.215 | 1.00 | 178.83 |
| 1974 | NZ | LYS A | 59 | 6.932 | 19.856 | −16.725 | 1.00 | 178.83 |
| 1975 | C | LYS A | 59 | 1.782 | 23.569 | −12.623 | 1.00 | 74.98 |
| 1976 | O | LYS A | 59 | 1.878 | 23.163 | −11.463 | 1.00 | 74.98 |
| 1977 | N | PHE A | 60 | 0.614 | 23.912 | −13.166 | 1.00 | 60.66 |
| 1978 | CA | PHE A | 60 | −0.640 | 23.780 | −12.418 | 1.00 | 60.66 |
| 1979 | CB | PHE A | 60 | −1.815 | 23.834 | −13.371 | 1.00 | 124.29 |
| 1980 | CG | PHE A | 60 | −1.949 | 25.140 | −14.046 | 1.00 | 124.29 |
| 1981 | CD1 | PHE A | 60 | −2.524 | 25.234 | −15.301 | 1.00 | 124.29 |
| 1982 | CD2 | PHE A | 60 | −1.510 | 26.294 | −13.425 | 1.00 | 124.29 |
| 1983 | CE1 | PHE A | 60 | −2.653 | 26.464 | −15.942 | 1.00 | 124.29 |
| 1984 | CE2 | PHE A | 60 | −1.630 | 27.527 | −14.054 | 1.00 | 124.29 |
| 1985 | CZ | PHE A | 60 | −2.209 | 27.613 | −15.313 | 1.00 | 124.29 |
| 1986 | C | PHE A | 60 | −0.714 | 22.496 | −11.595 | 1.00 | 60.66 |
| 1987 | O | PHE A | 60 | −1.287 | 22.487 | −10.504 | 1.00 | 60.66 |
| 1988 | N | GLU A | 61 | −0.124 | 21.418 | −12.112 | 1.00 | 94.84 |
| 1989 | CA | GLU A | 61 | −0.129 | 20.123 | −11.433 | 1.00 | 94.84 |
| 1990 | CB | GLU A | 61 | 0.502 | 19.037 | −12.312 | 1.00 | 214.43 |
| 1991 | CG | GLU A | 61 | −0.208 | 18.784 | −13.625 | 1.00 | 214.43 |
| 1992 | CD | GLU A | 61 | −0.246 | 20.011 | −14.508 | 1.00 | 214.43 |
| 1993 | OE1 | GLU A | 61 | 0.831 | 20.583 | −14.781 | 1.00 | 214.43 |
| 1994 | OE2 | GLU A | 61 | −1.352 | 20.403 | −14.930 | 1.00 | 214.43 |
| 1995 | C | GLU A | 61 | 0.626 | 20.165 | −10.114 | 1.00 | 94.84 |
| 1996 | O | GLU A | 61 | 0.397 | 19.318 | −9.253 | 1.00 | 94.84 |
| 1997 | N | ASP A | 62 | 1.535 | 21.130 | −9.959 | 1.00 | 76.23 |
| 1998 | CA | ASP A | 62 | 2.303 | 21.242 | −8.728 | 1.00 | 76.23 |
| 1999 | CB | ASP A | 62 | 3.493 | 22.175 | −8.913 | 1.00 | 161.53 |
| 2000 | CG | ASP A | 62 | 4.380 | 21.755 | −10.072 | 1.00 | 161.53 |
| 2001 | OD1 | ASP A | 62 | 4.571 | 20.536 | −10.273 | 1.00 | 161.53 |
| 2002 | OD2 | ASP A | 62 | 4.897 | 22.644 | −10.778 | 1.00 | 161.53 |
| 2003 | C | ASP A | 62 | 1.407 | 21.732 | −7.614 | 1.00 | 76.23 |
| 2004 | O | ASP A | 62 | 1.721 | 21.544 | −6.451 | 1.00 | 76.23 |
| 2005 | N | SER A | 63 | 0.280 | 22.341 | −7.977 | 1.00 | 83.22 |
| 2006 | CA | SER A | 63 | −0.680 | 22.828 | −6.992 | 1.00 | 83.22 |
| 2007 | CB | SER A | 63 | −1.880 | 23.464 | −7.691 | 1.00 | 115.03 |
| 2008 | OG | SER A | 63 | −1.503 | 24.633 | −8.399 | 1.00 | 115.03 |
| 2009 | C | SER A | 63 | −1.140 | 21.621 | −6.212 | 1.00 | 83.22 |
| 2010 | O | SER A | 63 | −1.508 | 20.640 | −6.814 | 1.00 | 83.22 |
| 2011 | N | GLY A | 64 | −1.124 | 21.660 | −4.887 | 1.00 | 65.94 |
| 2012 | CA | GLY A | 64 | −1.575 | 20.488 | −4.154 | 1.00 | 65.94 |
| 2013 | C | GLY A | 64 | −1.306 | 20.493 | −2.661 | 1.00 | 65.94 |
| 2014 | O | GLY A | 64 | −0.942 | 21.530 | −2.082 | 1.00 | 65.94 |
| 2015 | N | GLU A | 65 | −1.509 | 19.337 | −2.032 | 1.00 | 82.22 |
| 2016 | CA | GLU A | 65 | −1.285 | 19.159 | −0.605 | 1.00 | 82.22 |
| 2017 | CB | GLU A | 65 | −2.463 | 18.376 | −0.031 | 1.00 | 143.82 |
| 2018 | CG | GLU A | 65 | −2.304 | 17.897 | 1.394 | 1.00 | 143.82 |
| 2019 | CD | GLU A | 65 | −3.356 | 16.866 | 1.773 | 1.00 | 143.82 |
| 2020 | OE1 | GLU A | 65 | −3.374 | 15.779 | 1.157 | 1.00 | 143.82 |
| 2021 | OE2 | GLU A | 65 | −4.169 | 17.139 | 2.681 | 1.00 | 143.82 |
| 2022 | C | GLU A | 65 | 0.035 | 18.378 | −0.420 | 1.00 | 82.22 |
| 2023 | O | GLU A | 65 | 0.207 | 17.313 | −1.011 | 1.00 | 82.22 |
| 2024 | N | TYR A | 66 | 0.971 | 18.903 | 0.374 | 1.00 | 76.24 |
| 2025 | CA | TYR A | 66 | 2.240 | 18.224 | 0.614 | 1.00 | 76.24 |
| 2026 | CB | TYR A | 66 | 3.377 | 19.083 | 0.150 | 1.00 | 67.69 |
| 2027 | CG | TYR A | 66 | 3.426 | 19.339 | −1.314 | 1.00 | 67.69 |
| 2028 | CD1 | TYR A | 66 | 2.574 | 20.255 | −1.915 | 1.00 | 67.69 |
| 2029 | CE1 | TYR A | 66 | 2.680 | 20.572 | −3.265 | 1.00 | 67.69 |
| 2030 | CD2 | TYR A | 66 | 4.385 | 18.724 | −2.095 | 1.00 | 67.69 |
| 2031 | CE2 | TYR A | 66 | 4.502 | 19.017 | −3.447 | 1.00 | 67.69 |
| 2032 | CZ | TYR A | 66 | 3.647 | 19.948 | −4.032 | 1.00 | 67.69 |
| 2033 | OH | TYR A | 66 | 3.792 | 20.230 | −5.378 | 1.00 | 67.69 |
| 2034 | C | TYR A | 66 | 2.490 | 17.934 | 2.083 | 1.00 | 76.24 |
| 2035 | O | TYR A | 66 | 1.891 | 18.570 | 2.941 | 1.00 | 76.24 |
| 2036 | N | LYS A | 67 | 3.398 | 17.000 | 2.375 | 1.00 | 93.48 |
| 2037 | CA | LYS A | 67 | 3.756 | 16.664 | 3.759 | 1.00 | 93.48 |
| 2038 | CB | LYS A | 67 | 2.619 | 15.924 | 4.439 | 1.00 | 143.97 |
| 2039 | CG | LYS A | 67 | 2.079 | 14.788 | 3.619 | 1.00 | 143.97 |
| 2040 | CD | LYS A | 67 | 0.876 | 14.176 | 4.291 | 1.00 | 143.97 |
| 2041 | CE | LYS A | 67 | 0.213 | 13.163 | 3.385 | 1.00 | 143.97 |
| 2042 | NZ | LYS A | 67 | −1.009 | 12.616 | 4.023 | 1.00 | 143.97 |
| 2043 | C | LYS A | 67 | 5.011 | 15.818 | 3.806 | 1.00 | 93.48 |
| 2044 | O | LYS A | 67 | 5.357 | 15.166 | 2.824 | 1.00 | 93.48 |
| 2045 | N | CYS A | 68 | 5.715 | 15.852 | 4.932 | 1.00 | 71.26 |
| 2046 | CA | CYS A | 68 | 6.914 | 15.044 | 5.067 | 1.00 | 71.26 |
| 2047 | C | CYS A | 68 | 6.823 | 14.232 | 6.340 | 1.00 | 71.26 |
| 2048 | O | CYS A | 68 | 6.020 | 14.540 | 7.208 | 1.00 | 71.26 |
| 2049 | CB | CYS A | 68 | 8.183 | 15.905 | 5.041 | 1.00 | 93.73 |
| 2050 | SG | CYS A | 68 | 8.385 | 17.184 | 6.305 | 1.00 | 93.73 |
| 2051 | N | GLN A | 69 | 7.619 | 13.174 | 6.425 | 1.00 | 106.93 |
| 2052 | CA | GLN A | 69 | 7.651 | 12.302 | 7.591 | 1.00 | 106.93 |
| 2053 | CB | GLN A | 69 | 6.558 | 11.233 | 7.476 | 1.00 | 95.79 |
| 2054 | CG | GLN A | 69 | 6.744 | 10.032 | 8.390 | 1.00 | 95.79 |
| 2055 | CD | GLN A | 69 | 5.702 | 8.954 | 8.161 | 1.00 | 95.79 |
| 2056 | OE1 | GLN A | 69 | 5.476 | 8.521 | 7.024 | 1.00 | 95.79 |
| 2057 | NE2 | GLN A | 69 | 5.060 | 8.509 | 9.244 | 1.00 | 95.79 |
| 2058 | C | GLN A | 69 | 9.015 | 11.641 | 7.629 | 1.00 | 106.93 |
| 2059 | O | GLN A | 69 | 9.657 | 11.496 | 6.594 | 1.00 | 106.93 |
| 2060 | N | HIS A | 70 | 9.462 | 11.243 | 8.813 | 1.00 | 174.41 |
| 2061 | CA | HIS A | 70 | 10.753 | 10.589 | 8.928 | 1.00 | 174.41 |
| 2062 | CB | HIS A | 70 | 11.601 | 11.296 | 9.977 | 1.00 | 160.27 |
| 2063 | CG | HIS A | 70 | 12.022 | 12.673 | 9.572 | 1.00 | 160.27 |
| 2064 | CD2 | HIS A | 70 | 11.502 | 13.885 | 9.873 | 1.00 | 160.27 |
| 2065 | ND1 | HIS A | 70 | 13.085 | 12.909 | 8.726 | 1.00 | 160.27 |
| 2066 | CE1 | HIS A | 70 | 13.203 | 14.210 | 8.527 | 1.00 | 160.27 |
| 2067 | NE2 | HIS A | 70 | 12.257 | 14.824 | 9.213 | 1.00 | 160.27 |
| 2068 | C | HIS A | 70 | 10.632 | 9.112 | 9.268 | 1.00 | 174.41 |
| 2069 | O | HIS A | 70 | 9.543 | 8.536 | 9.237 | 1.00 | 174.41 |
| 2070 | N | GLN A | 71 | 11.764 | 8.505 | 9.590 | 1.00 | 242.81 |
| 2071 | CA | GLN A | 71 | 11.815 | 7.091 | 9.923 | 1.00 | 242.81 |
| 2072 | CB | GLN A | 71 | 13.246 | 6.724 | 10.335 | 1.00 | 199.62 |
| 2073 | CG | GLN A | 71 | 13.632 | 5.293 | 9.992 | 1.00 | 199.62 |
| 2074 | CD | GLN A | 71 | 13.345 | 4.945 | 8.543 | 1.00 | 199.62 |
| 2075 | OE1 | GLN A | 71 | 14.015 | 5.423 | 7.634 | 1.00 | 199.62 |
| 2076 | NE2 | GLN A | 71 | 12.331 | 4.115 | 8.324 | 1.00 | 199.62 |
| 2077 | C | GLN A | 71 | 10.817 | 6.722 | 11.027 | 1.00 | 242.81 |
| 2078 | O | GLN A | 71 | 9.989 | 5.829 | 10.844 | 1.00 | 242.81 |
| 2079 | N | GLN A | 72 | 10.886 | 7.419 | 12.160 | 1.00 | 160.50 |
| 2080 | CA | GLN A | 72 | 9.991 | 7.143 | 13.289 | 1.00 | 160.50 |
| 2081 | CB | GLN A | 72 | 10.803 | 6.584 | 14.465 | 1.00 | 249.38 |
| 2082 | CG | GLN A | 72 | 9.972 | 6.150 | 15.671 | 1.00 | 249.38 |
| 2083 | CD | GLN A | 72 | 10.819 | 5.563 | 16.791 | 1.00 | 249.38 |
| 2084 | OE1 | GLN A | 72 | 11.537 | 4.581 | 16.594 | 1.00 | 249.38 |
| 2085 | NE2 | GLN A | 72 | 10.738 | 6.164 | 17.975 | 1.00 | 249.38 |
| 2086 | C | GLN A | 72 | 9.237 | 8.392 | 13.740 | 1.00 | 160.50 |
| 2087 | O | GLN A | 72 | 9.319 | 8.797 | 14.901 | 1.00 | 160.50 |
| 2088 | N | VAL A | 73 | 8.493 | 9.001 | 12.825 | 1.00 | 139.31 |
| 2089 | CA | VAL A | 73 | 7.759 | 10.217 | 13.154 | 1.00 | 139.31 |
| 2090 | CB | VAL A | 73 | 8.575 | 11.467 | 12.795 | 1.00 | 182.81 |
| 2091 | CG1 | VAL A | 73 | 7.960 | 12.688 | 13.430 | 1.00 | 182.81 |
| 2092 | CG2 | VAL A | 73 | 10.000 | 11.297 | 13.237 | 1.00 | 182.81 |
| 2093 | C | VAL A | 73 | 6.445 | 10.284 | 12.391 | 1.00 | 139.31 |
| 2094 | O | VAL A | 73 | 6.352 | 9.819 | 11.254 | 1.00 | 139.31 |
| 2095 | N | ASN A | 74 | 5.428 | 10.864 | 13.019 | 1.00 | 98.24 |
| 2096 | CA | ASN A | 74 | 4.136 | 10.988 | 12.376 | 1.00 | 98.24 |
| 2097 | CB | ASN A | 74 | 3.045 | 11.209 | 13.427 | 1.00 | 227.24 |
| 2098 | CG | ASN A | 74 | 3.039 | 10.124 | 14.489 | 1.00 | 227.24 |
| 2099 | OD1 | ASN A | 74 | 3.176 | 8.940 | 14.170 | 1.00 | 227.24 |
| 2100 | ND2 | ASN A | 74 | 2.875 | 10.520 | 15.748 | 1.00 | 227.24 |
| 2101 | C | ASN A | 74 | 4.194 | 12.144 | 11.378 | 1.00 | 98.24 |
| 2102 | O | ASN A | 74 | 4.649 | 13.246 | 11.700 | 1.00 | 98.24 |
| 2103 | N | GLU A | 75 | 3.750 | 11.863 | 10.157 | 1.00 | 124.76 |
| 2104 | CA | GLU A | 75 | 3.730 | 12.842 | 9.074 | 1.00 | 124.76 |
| 2105 | CB | GLU A | 75 | 2.881 | 12.302 | 7.921 | 1.00 | 249.33 |
| 2106 | CG | GLU A | 75 | 1.709 | 11.440 | 8.364 | 1.00 | 249.33 |
| 2107 | CD | GLU A | 75 | 1.032 | 10.734 | 7.202 | 1.00 | 249.33 |
| 2108 | OE1 | GLU A | 75 | 1.730 | 10.023 | 6.446 | 1.00 | 249.33 |
| 2109 | OE2 | GLU A | 75 | 0.198 | 10.888 | 7.048 | 1.00 | 249.33 |
| 2110 | C | GLU A | 75 | 3.245 | 14.232 | 9.499 | 1.00 | 124.76 |
| 2111 | O | GLU A | 75 | 2.346 | 14.372 | 10.327 | 1.00 | 124.76 |
| 2112 | N | SER A | 76 | 3.859 | 15.255 | 8.912 | 1.00 | 84.02 |
| 2113 | CA | SER A | 76 | 3.569 | 16.653 | 9.208 | 1.00 | 84.02 |
| 2114 | CB | SER A | 76 | 4.578 | 17.534 | 8.509 | 1.00 | 92.60 |
| 2115 | OG | SER A | 76 | 4.391 | 17.395 | 7.108 | 1.00 | 92.60 |
| 2116 | C | SER A | 76 | 2.201 | 17.096 | 8.754 | 1.00 | 84.02 |
| 2117 | O | SER A | 76 | 1.599 | 16.468 | 7.888 | 1.00 | 84.02 |
| 2118 | N | GLU A | 77 | 1.722 | 18.198 | 9.323 | 1.00 | 82.56 |
| 2119 | CA | GLU A | 77 | 0.415 | 18.751 | 8.960 | 1.00 | 82.56 |
| 2120 | CB | GLU A | 77 | 0.055 | 19.918 | 9.883 | 1.00 | 211.53 |
| 2121 | CG | GLU A | 77 | −0.157 | 19.511 | 11.331 | 1.00 | 211.53 |
| 2122 | CD | GLU A | 77 | −1.343 | 18.579 | 11.512 | 1.00 | 211.53 |
| 2123 | OE1 | GLU A | 77 | −1.831 | 18.020 | 10.505 | 1.00 | 211.53 |
| 2124 | OE2 | GLU A | 77 | −1.780 | 18.396 | 12.668 | 1.00 | 211.53 |
| 2125 | C | GLU A | 77 | 0.550 | 19.239 | 7.533 | 1.00 | 82.56 |
| 2126 | O | GLU A | 77 | 1.397 | 20.102 | 7.252 | 1.00 | 82.56 |
| 2127 | N | PRO A | 78 | −0.250 | 18.679 | 6.604 | 1.00 | 57.51 |
| 2128 | CD | PRO A | 78 | −1.105 | 17.493 | 6.808 | 1.00 | 210.77 |
| 2129 | CA | PRO A | 78 | −0.226 | 19.047 | 5.186 | 1.00 | 57.51 |
| 2130 | CB | PRO A | 78 | −1.469 | 18.370 | 4.644 | 1.00 | 210.77 |
| 2131 | CG | PRO A | 78 | −1.440 | 17.076 | 5.376 | 1.00 | 210.77 |
| 2132 | C | PRO A | 78 | −0.193 | 20.544 | 4.936 | 1.00 | 57.51 |
| 2133 | O | PRO A | 78 | −0.607 | 21.338 | 5.785 | 1.00 | 57.51 |
| 2134 | N | VAL A | 79 | 0.343 | 20.931 | 3.789 | 1.00 | 75.93 |
| 2135 | CA | VAL A | 79 | 0.396 | 22.331 | 3.422 | 1.00 | 75.93 |
| 2136 | CB | VAL A | 79 | 1.780 | 22.859 | 3.574 | 1.00 | 49.48 |
| 2137 | CG1 | VAL A | 79 | 1.916 | 24.215 | 2.850 | 1.00 | 49.48 |
| 2138 | CG2 | VAL A | 79 | 2.078 | 23.010 | 5.039 | 1.00 | 49.48 |
| 2139 | C | VAL A | 79 | −0.033 | 22.466 | 1.972 | 1.00 | 75.93 |
| 2140 | O | VAL A | 79 | 0.463 | 21.748 | 1.113 | 1.00 | 75.93 |
| 2141 | N | TYR A | 80 | −0.961 | 23.375 | 1.696 | 1.00 | 60.67 |
| 2142 | CA | TYR A | 80 | −1.424 | 23.519 | 0.336 | 1.00 | 60.67 |
| 2143 | CB | TYR A | 80 | −2.903 | 23.814 | 0.280 | 1.00 | 249.12 |
| 2144 | CG | TYR A | 80 | −3.420 | 23.538 | −1.115 | 1.00 | 249.12 |
| 2145 | CD1 | TYR A | 80 | −3.434 | 22.256 | −1.623 | 1.00 | 249.12 |
| 2146 | CE1 | TYR A | 80 | −3.870 | 22.005 | −2.920 | 1.00 | 249.12 |
| 2147 | CD2 | TYR A | 80 | −3.902 | 24.575 | −1.927 | 1.00 | 249.12 |
| 2148 | CE2 | TYR A | 80 | −4.414 | 24.332 | −3.216 | 1.00 | 249.12 |
| 2149 | CZ | TYR A | 80 | −4.378 | 23.015 | −3.703 | 1.00 | 249.12 |
| 2150 | OH | TYR A | 80 | −4.926 | 22.722 | −4.929 | 1.00 | 249.12 |
| 2151 | C | TYR A | 80 | −0.736 | 24.582 | −0.438 | 1.00 | 60.67 |
| 2152 | O | TYR A | 80 | −0.537 | 25.688 | 0.043 | 1.00 | 60.67 |
| 2153 | N | LEU A | 81 | −0.414 | 24.264 | −1.669 | 1.00 | 53.62 |
| 2154 | CA | LEU A | 81 | 0.237 | 25.227 | −2.520 | 1.00 | 53.62 |
| 2155 | CB | LEU A | 81 | 1.547 | 24.619 | −3.003 | 1.00 | 66.18 |
| 2156 | CG | LEU A | 81 | 2.237 | 25.486 | −4.035 | 1.00 | 66.18 |
| 2157 | CD1 | LEU A | 81 | 2.603 | 26.806 | −3.373 | 1.00 | 66.18 |
| 2158 | CD2 | LEU A | 81 | 3.461 | 24.803 | −4.566 | 1.00 | 66.18 |
| 2159 | C | LEU A | 81 | −0.703 | 25.487 | −3.698 | 1.00 | 53.62 |
| 2160 | O | LEU A | 81 | −1.229 | 24.534 | −4.283 | 1.00 | 53.62 |
| 2161 | N | GLU A | 82 | −0.956 | 26.742 | −4.048 | 1.00 | 63.15 |
| 2162 | CA | GLU A | 82 | −1.821 | 26.990 | −5.201 | 1.00 | 63.15 |
| 2163 | CB | GLU A | 82 | −3.099 | 27.700 | −4.772 | 1.00 | 149.46 |
| 2164 | CG | GLU A | 82 | −4.259 | 27.463 | −5.722 | 1.00 | 149.46 |
| 2165 | CD | GLU A | 82 | −5.537 | 28.157 | −5.276 | 1.00 | 149.46 |
| 2166 | OE1 | GLU A | 82 | −5.798 | 28.194 | −4.050 | 1.00 | 149.46 |
| 2167 | OE2 | GLU A | 82 | −6.286 | 28.653 | −6.151 | 1.00 | 149.46 |
| 2168 | C | GLU A | 82 | −1.100 | 27.823 | −6.283 | 1.00 | 63.15 |
| 2169 | O | GLU A | 82 | −0.503 | 28.878 | −5.996 | 1.00 | 63.15 |
| 2170 | N | VAL A | 83 | −1.157 | 27.352 | −7.526 | 1.00 | 58.52 |
| 2171 | CA | VAL A | 83 | −0.517 | 28.050 | −8.632 | 1.00 | 58.52 |
| 2172 | CB | VAL A | 83 | 0.194 | 27.083 | −9.516 | 1.00 | 61.79 |
| 2173 | CG1 | VAL A | 83 | 0.749 | 27.819 | −10.728 | 1.00 | 61.79 |
| 2174 | CG2 | VAL A | 83 | 1.294 | 26.427 | −8.738 | 1.00 | 61.79 |
| 2175 | C | VAL A | 83 | −1.473 | 28.859 | −9.501 | 1.00 | 58.52 |
| 2176 | O | VAL A | 83 | −2.540 | 28.364 | −9.877 | 1.00 | 58.52 |
| 2177 | N | PHE A | 84 | −1.082 | 30.088 | −9.839 | 1.00 | 70.51 |
| 2178 | CA | PHE A | 84 | −1.947 | 30.947 | −10.632 | 1.00 | 70.51 |
| 2179 | CB | PHE A | 84 | −2.395 | 32.164 | −9.834 | 1.00 | 69.94 |
| 2180 | CG | PHE A | 84 | −3.130 | 31.836 | −8.588 | 1.00 | 69.94 |
| 2181 | CD1 | PHE A | 84 | −2.455 | 31.374 | −7.488 | 1.00 | 69.94 |
| 2182 | CD2 | PHE A | 84 | −4.503 | 32.031 | −8.498 | 1.00 | 69.94 |
| 2183 | CE1 | PHE A | 84 | −3.134 | 31.108 | −6.323 | 1.00 | 69.94 |
| 2184 | CE2 | PHE A | 84 | −5.199 | 31.764 | −7.324 | 1.00 | 69.94 |
| 2185 | CZ | PHE A | 84 | −4.521 | 31.312 | −6.242 | 1.00 | 69.94 |
| 2186 | C | PHE A | 84 | −1.390 | 31.480 | −11.923 | 1.00 | 70.51 |
| 2187 | O | PHE A | 84 | −0.186 | 31.452 | −12.179 | 1.00 | 70.51 |
| 2188 | N | SER A | 85 | −2.327 | 31.985 | −12.717 | 1.00 | 86.88 |
| 2189 | CA | SER A | 85 | −2.067 | 32.625 | −13.989 | 1.00 | 86.88 |
| 2190 | CB | SER A | 85 | −2.453 | 31.714 | −15.142 | 1.00 | 135.23 |
| 2191 | OG | SER A | 85 | −2.214 | 32.358 | −16.378 | 1.00 | 135.23 |
| 2192 | C | SER A | 85 | −2.999 | 33.835 | −13.959 | 1.00 | 86.88 |
| 2193 | O | SER A | 85 | −4.226 | 33.670 | −14.007 | 1.00 | 86.88 |
| 2194 | N | ASP A | 86 | −2.425 | 35.033 | −13.836 | 1.00 | 47.41 |
| 2195 | CA | ASP A | 86 | −3.209 | 36.256 | −13.803 | 1.00 | 47.41 |
| 2196 | CB | ASP A | 86 | −4.131 | 36.259 | −12.589 | 1.00 | 131.95 |
| 2197 | CG | ASP A | 86 | −5.454 | 36.927 | −12.876 | 1.00 | 131.95 |
| 2198 | OD1 | ASP A | 86 | −5.433 | 38.087 | −13.345 | 1.00 | 131.95 |
| 2199 | OD2 | ASP A | 86 | −6.509 | 36.296 | −12.629 | 1.00 | 131.95 |
| 2200 | C | ASP A | 86 | −2.245 | 37.453 | −13.756 | 1.00 | 47.41 |
| 2201 | O | ASP A | 86 | −1.043 | 37.284 | −13.502 | 1.00 | 47.41 |
| 2202 | N | TRP A | 87 | −2.760 | 38.661 | −14.004 | 1.00 | 62.18 |
| 2203 | CA | TRP A | 87 | −1.903 | 39.848 | −14.009 | 1.00 | 62.18 |
| 2204 | CB | TRP A | 87 | −2.668 | 41.090 | −14.457 | 1.00 | 225.09 |
| 2205 | CG | TRP A | 87 | −2.632 | 41.233 | −15.914 | 1.00 | 225.09 |
| 2206 | CD2 | TRP A | 87 | −3.596 | 40.723 | −16.830 | 1.00 | 225.09 |
| 2207 | CE2 | TRP A | 87 | −3.100 | 40.950 | −18.122 | 1.00 | 225.09 |
| 2208 | CE3 | TRP A | 87 | −4.834 | 40.077 | −16.683 | 1.00 | 225.09 |
| 2209 | CD1 | TRP A | 87 | −1.618 | 41.757 | −16.666 | 1.00 | 225.09 |
| 2210 | NE1 | TRP A | 87 | −1.891 | 41.586 | −17.994 | 1.00 | 225.09 |
| 2211 | CZ2 | TRP A | 87 | −3.794 | 40.549 | −19.261 | 1.00 | 225.09 |
| 2212 | CZ3 | TRP A | 87 | −5.528 | 39.687 | −17.820 | 1.00 | 225.09 |
| 2213 | CH2 | TRP A | 87 | −5.008 | 39.923 | −19.086 | 1.00 | 225.09 |
| 2214 | C | TRP A | 87 | −1.350 | 40.068 | −12.645 | 1.00 | 62.18 |
| 2215 | O | TRP A | 87 | −0.139 | 40.149 | −12.468 | 1.00 | 62.18 |
| 2216 | N | LEU A | 88 | −2.249 | 40.140 | −11.673 | 1.00 | 74.08 |
| 2217 | CA | LEU A | 88 | −1.863 | 40.372 | −10.295 | 1.00 | 74.08 |
| 2218 | CB | LEU A | 88 | −2.457 | 41.681 | −9.805 | 1.00 | 87.26 |
| 2219 | CG | LEU A | 88 | −1.907 | 42.914 | −10.492 | 1.00 | 87.26 |
| 2220 | CD1 | LEU A | 88 | −2.496 | 44.139 | −9.837 | 1.00 | 87.26 |
| 2221 | CD2 | LEU A | 88 | −0.394 | 42.908 | −10.383 | 1.00 | 87.26 |
| 2222 | C | LEU A | 88 | −2.305 | 39.274 | −9.369 | 1.00 | 74.08 |
| 2223 | O | LEU A | 88 | −3.399 | 38.723 | −9.501 | 1.00 | 74.08 |
| 2224 | N | LEU A | 89 | −1.466 | 38.978 | −8.399 | 1.00 | 49.26 |
| 2225 | CA | LEU A | 89 | −1.769 | 37.943 | −7.432 | 1.00 | 49.26 |
| 2226 | CB | LEU A | 89 | −0.902 | 36.718 | −7.675 | 1.00 | 70.28 |
| 2227 | CG | LEU A | 89 | −1.170 | 35.653 | −6.637 | 1.00 | 70.28 |
| 2228 | CD1 | LEU A | 89 | −2.692 | 35.436 | −6.511 | 1.00 | 70.28 |
| 2229 | CD2 | LEU A | 89 | −0.455 | 34.401 | −7.046 | 1.00 | 70.28 |
| 2230 | C | LEU A | 89 | −1.499 | 38.470 | −6.036 | 1.00 | 49.26 |
| 2231 | O | LEU A | 89 | −0.429 | 39.008 | −5.784 | 1.00 | 49.26 |
| 2232 | N | LEU A | 90 | −2.459 | 38.342 | −5.127 | 1.00 | 72.68 |
| 2233 | CA | LEU A | 90 | −2.240 | 38.815 | −3.760 | 1.00 | 72.68 |
| 2234 | CB | LEU A | 90 | −3.562 | 39.231 | −3.111 | 1.00 | 33.75 |
| 2235 | CG | LEU A | 90 | −3.444 | 39.630 | −1.648 | 1.00 | 33.75 |
| 2236 | CD1 | LEU A | 90 | −2.488 | 40.814 | −1.620 | 1.00 | 33.75 |
| 2237 | CD2 | LEU A | 90 | −4.790 | 40.011 | −1.047 | 1.00 | 33.75 |
| 2238 | C | LEU A | 90 | −1.623 | 37.701 | −2.931 | 1.00 | 72.68 |
| 2239 | O | LEU A | 90 | −2.254 | 36.674 | −2.710 | 1.00 | 72.68 |
| 2240 | N | GLN A | 91 | −0.398 | 37.896 | −2.462 | 1.00 | 48.17 |
| 2241 | CA | GLN A | 91 | 0.255 | 36.864 | −1.656 | 1.00 | 48.17 |
| 2242 | CB | GLN A | 91 | 1.692 | 36.682 | −2.110 | 1.00 | 50.84 |
| 2243 | CG | GLN A | 91 | 1.773 | 36.315 | −3.559 | 1.00 | 50.84 |
| 2244 | CD | GLN A | 91 | 3.159 | 35.954 | −3.971 | 1.00 | 50.84 |
| 2245 | OE1 | GLN A | 91 | 4.041 | 36.801 | −4.013 | 1.00 | 50.84 |
| 2246 | NE2 | GLN A | 91 | 3.371 | 34.688 | −4.271 | 1.00 | 50.84 |
| 2247 | C | GLN A | 91 | 0.218 | 37.151 | −0.165 | 1.00 | 48.17 |
| 2248 | O | GLN A | 91 | 0.282 | 38.298 | 0.254 | 1.00 | 48.17 |
| 2249 | N | ALA A | 92 | 0.098 | 36.113 | 0.648 | 1.00 | 56.37 |
| 2250 | CA | ALA A | 92 | 0.044 | 36.326 | 2.080 | 1.00 | 56.37 |
| 2251 | CB | ALA A | 92 | −1.329 | 36.039 | 2.579 | 1.00 | 37.31 |
| 2252 | C | ALA A | 92 | 1.033 | 35.422 | 2.769 | 1.00 | 56.37 |
| 2253 | O | ALA A | 92 | 1.202 | 34.266 | 2.381 | 1.00 | 56.37 |
| 2254 | N | SER A | 93 | 1.695 | 35.939 | 3.794 | 1.00 | 55.78 |
| 2255 | CA | SER A | 93 | 2.665 | 35.146 | 4.535 | 1.00 | 55.78 |
| 2256 | CB | SER A | 93 | 3.171 | 35.909 | 5.763 | 1.00 | 74.91 |
| 2257 | OG | SER A | 93 | 2.111 | 36.461 | 6.531 | 1.00 | 74.91 |
| 2258 | C | SER A | 93 | 1.912 | 33.919 | 4.956 | 1.00 | 55.78 |
| 2259 | O | SER A | 93 | 2.205 | 32.828 | 4.501 | 1.00 | 55.78 |
| 2260 | N | ALA A | 94 | 0.904 | 34.112 | 5.796 | 1.00 | 63.55 |
| 2261 | CA | ALA A | 94 | 0.070 | 33.021 | 6.287 | 1.00 | 63.55 |
| 2262 | CB | ALA A | 94 | 0.410 | 32.712 | 7.734 | 1.00 | 137.30 |
| 2263 | C | ALA A | 94 | −1.392 | 33.445 | 6.162 | 1.00 | 63.55 |
| 2264 | O | ALA A | 94 | −1.713 | 34.616 | 6.341 | 1.00 | 63.55 |
| 2265 | N | GLU A | 95 | −2.283 | 32.501 | 5.856 | 1.00 | 58.25 |
| 2266 | CA | GLU A | 95 | −3.702 | 32.826 | 5.684 | 1.00 | 58.25 |
| 2267 | CB | GLU A | 95 | −4.344 | 31.866 | 4.701 | 1.00 | 138.90 |
| 2268 | CG | GLU A | 95 | −3.695 | 31.890 | 3.337 | 1.00 | 138.90 |
| 2269 | CD | GLU A | 95 | −4.541 | 31.214 | 2.269 | 1.00 | 138.90 |
| 2270 | OE1 | GLU A | 95 | −4.085 | 31.137 | 1.108 | 1.00 | 138.90 |
| 2271 | OE2 | GLU A | 95 | −5.664 | 30.763 | 2.584 | 1.00 | 138.90 |
| 2272 | C | GLU A | 95 | −4.494 | 32.844 | 6.979 | 1.00 | 58.25 |
| 2273 | O | GLU A | 95 | −5.600 | 33.361 | 7.016 | 1.00 | 58.25 |
| 2274 | N | VAL A | 96 | −3.934 | 32.267 | 8.040 | 1.00 | 62.67 |
| 2275 | CA | VAL A | 96 | −4.584 | 32.253 | 9.353 | 1.00 | 62.67 |
| 2276 | CB | VAL A | 96 | −5.180 | 30.912 | 9.637 | 1.00 | 62.13 |
| 2277 | CG1 | VAL A | 96 | −6.169 | 31.021 | 10.762 | 1.00 | 62.13 |
| 2278 | CG2 | VAL A | 96 | −5.835 | 30.401 | 8.402 | 1.00 | 62.13 |
| 2279 | C | VAL A | 96 | −3.512 | 32.568 | 10.386 | 1.00 | 62.67 |
| 2280 | O | VAL A | 96 | −2.422 | 31.999 | 10.335 | 1.00 | 62.67 |
| 2281 | N | VAL A | 97 | −3.829 | 33.449 | 11.333 | 1.00 | 50.85 |
| 2282 | CA | VAL A | 97 | −2.833 | 33.902 | 12.289 | 1.00 | 50.85 |
| 2283 | CB | VAL A | 97 | −2.307 | 35.276 | 11.860 | 1.00 | 70.57 |
| 2284 | CG1 | VAL A | 97 | −1.069 | 35.609 | 12.633 | 1.00 | 70.57 |
| 2285 | CG2 | VAL A | 97 | −2.063 | 35.319 | 10.372 | 1.00 | 70.57 |
| 2286 | C | VAL A | 97 | −3.285 | 34.077 | 13.723 | 1.00 | 50.85 |
| 2287 | O | VAL A | 97 | −4.373 | 34.653 | 13.953 | 1.00 | 50.85 |
| 2288 | N | MET A | 98 | −2.449 | 33.629 | 14.673 | 1.00 | 73.49 |
| 2289 | CA | MET A | 98 | −2.749 | 33.780 | 16.096 | 1.00 | 73.49 |
| 2290 | CB | MET A | 98 | −1.766 | 32.956 | 16.916 | 1.00 | 228.45 |
| 2291 | CG | MET A | 98 | −1.855 | 31.478 | 16.645 | 1.00 | 228.45 |
| 2292 | SD | MET A | 98 | −3.227 | 30.766 | 17.530 | 1.00 | 228.45 |
| 2293 | CE | MET A | 98 | −2.529 | 30.766 | 19.195 | 1.00 | 228.45 |
| 2294 | C | MET A | 98 | −2.617 | 35.276 | 16.477 | 1.00 | 73.49 |
| 2295 | O | MET A | 98 | −1.636 | 35.921 | 16.109 | 1.00 | 73.49 |
| 2296 | N | GLU A | 99 | −3.595 | 35.826 | 17.202 | 1.00 | 97.63 |
| 2297 | CA | GLU A | 99 | −3.546 | 37.228 | 17.603 | 1.00 | 97.63 |
| 2298 | CB | GLU A | 99 | −4.562 | 37.499 | 18.710 | 1.00 | 188.19 |
| 2299 | CG | GLU A | 99 | −4.954 | 38.958 | 18.826 | 1.00 | 188.19 |
| 2300 | CD | GLU A | 99 | −5.707 | 39.259 | 20.106 | 1.00 | 188.19 |
| 2301 | OE1 | GLU A | 99 | −6.524 | 38.412 | 20.529 | 1.00 | 188.19 |
| 2302 | OE2 | GLU A | 99 | −5.492 | 40.347 | 20.682 | 1.00 | 188.19 |
| 2303 | C | GLU A | 99 | −2.146 | 37.510 | 18.128 | 1.00 | 97.63 |
| 2304 | O | GLU A | 99 | −1.651 | 36.783 | 18.987 | 1.00 | 97.63 |
| 2305 | N | GLY A | 100 | −1.492 | 38.538 | 17.594 | 1.00 | 88.99 |
| 2306 | CA | GLY A | 100 | −0.159 | 38.881 | 18.066 | 1.00 | 88.99 |
| 2307 | C | GLY A | 100 | 0.992 | 38.577 | 17.130 | 1.00 | 88.99 |
| 2308 | O | GLY A | 100 | 2.071 | 39.135 | 17.293 | 1.00 | 88.99 |
| 2309 | N | GLN A | 101 | 0.777 | 37.699 | 16.154 | 1.00 | 57.71 |
| 2310 | CA | GLN A | 101 | 1.820 | 37.329 | 15.192 | 1.00 | 57.71 |
| 2311 | CB | GLN A | 101 | 1.568 | 35.933 | 14.652 | 1.00 | 91.13 |
| 2312 | CG | GLN A | 101 | 1.663 | 34.861 | 15.708 | 1.00 | 91.13 |
| 2313 | CD | GLN A | 101 | 2.932 | 34.976 | 16.532 | 1.00 | 91.13 |
| 2314 | OE1 | GLN A | 101 | 3.038 | 35.828 | 17.420 | 1.00 | 91.13 |
| 2315 | NE2 | GLN A | 101 | 3.912 | 34.131 | 16.230 | 1.00 | 91.13 |
| 2316 | C | GLN A | 101 | 1.973 | 38.281 | 14.017 | 1.00 | 57.71 |
| 2317 | O | GLN A | 101 | 1.117 | 39.124 | 13.763 | 1.00 | 57.71 |
| 2318 | N | PRO A | 102 | 3.070 | 38.153 | 13.266 | 1.00 | 73.79 |
| 2319 | CD | PRO A | 102 | 4.201 | 37.220 | 13.403 | 1.00 | 74.96 |
| 2320 | CA | PRO A | 102 | 3.264 | 39.049 | 12.130 | 1.00 | 73.79 |
| 2321 | CB | PRO A | 102 | 4.760 | 38.932 | 11.873 | 1.00 | 74.96 |
| 2322 | CG | PRO A | 102 | 5.018 | 37.499 | 12.139 | 1.00 | 74.96 |
| 2323 | C | PRO A | 102 | 2.425 | 38.610 | 10.940 | 1.00 | 73.79 |
| 2324 | O | PRO A | 102 | 2.053 | 37.446 | 10.831 | 1.00 | 73.79 |
| 2325 | N | LEU A | 103 | 2.125 | 39.551 | 10.054 | 1.00 | 77.13 |
| 2326 | CA | LEU A | 103 | 1.345 | 39.258 | 8.862 | 1.00 | 77.13 |
| 2327 | CB | LEU A | 103 | −0.101 | 39.627 | 9.094 | 1.00 | 77.95 |
| 2328 | CG | LEU A | 103 | −0.892 | 39.326 | 7.831 | 1.00 | 77.95 |
| 2329 | CD1 | LEU A | 103 | −0.843 | 37.836 | 7.584 | 1.00 | 77.95 |
| 2330 | CD2 | LEU A | 103 | −2.324 | 39.799 | 7.975 | 1.00 | 77.95 |
| 2331 | C | LEU A | 103 | 1.850 | 40.060 | 7.680 | 1.00 | 77.13 |
| 2332 | O | LEU A | 103 | 1.892 | 41.280 | 7.769 | 1.00 | 77.13 |
| 2333 | N | PHE A | 104 | 2.226 | 39.404 | 6.580 | 1.00 | 65.06 |
| 2334 | CA | PHE A | 104 | 2.708 | 40.147 | 5.410 | 1.00 | 65.06 |
| 2335 | CB | PHE A | 104 | 4.175 | 39.821 | 5.102 | 1.00 | 119.06 |
| 2336 | CG | PHE A | 104 | 5.118 | 40.096 | 6.246 | 1.00 | 119.06 |
| 2337 | CD1 | PHE A | 104 | 5.209 | 39.208 | 7.312 | 1.00 | 119.06 |
| 2338 | CD2 | PHE A | 104 | 5.926 | 41.237 | 6.255 | 1.00 | 119.06 |
| 2339 | CE1 | PHE A | 104 | 6.086 | 39.443 | 8.379 | 1.00 | 119.06 |
| 2340 | CE2 | PHE A | 104 | 6.811 | 41.486 | 7.321 | 1.00 | 119.06 |
| 2341 | CZ | PHE A | 104 | 6.891 | 40.585 | 8.382 | 1.00 | 119.06 |
| 2342 | C | PHE A | 104 | 1.869 | 39.886 | 4.164 | 1.00 | 65.06 |
| 2343 | O | PHE A | 104 | 1.640 | 38.741 | 3.816 | 1.00 | 65.06 |
| 2344 | N | LEU A | 105 | 1.373 | 40.944 | 3.519 | 1.00 | 48.39 |
| 2345 | CA | LEU A | 105 | 0.597 | 40.795 | 2.282 | 1.00 | 48.39 |
| 2346 | CB | LEU A | 105 | −0.708 | 41.544 | 2.354 | 1.00 | 38.52 |
| 2347 | CG | LEU A | 105 | −1.516 | 41.145 | 3.570 | 1.00 | 38.52 |
| 2348 | CD1 | LEU A | 105 | −2.952 | 41.785 | 3.515 | 1.00 | 38.52 |
| 2349 | CD2 | LEU A | 105 | −1.587 | 39.647 | 3.571 | 1.00 | 38.52 |
| 2350 | C | LEU A | 105 | 1.445 | 41.417 | 1.205 | 1.00 | 48.39 |
| 2351 | O | LEU A | 105 | 2.137 | 42.397 | 1.461 | 1.00 | 48.39 |
| 2352 | N | ARG A | 106 | 1.385 | 40.872 | 0.001 | 1.00 | 64.12 |
| 2353 | CA | ARG A | 106 | 2.198 | 41.394 | −1.074 | 1.00 | 64.12 |
| 2354 | CB | ARG A | 106 | 3.424 | 40.501 | −1.232 | 1.00 | 100.28 |
| 2355 | CG | ARG A | 106 | 4.313 | 40.873 | −2.370 | 1.00 | 100.28 |
| 2356 | CD | ARG A | 106 | 5.351 | 39.801 | −2.607 | 1.00 | 100.28 |
| 2357 | NE | ARG A | 106 | 6.190 | 40.124 | −3.755 | 1.00 | 100.28 |
| 2358 | CZ | ARG A | 106 | 6.892 | 39.234 | −4.443 | 1.00 | 100.28 |
| 2359 | NH1 | ARG A | 106 | 6.854 | 37.957 | −4.100 | 1.00 | 100.28 |
| 2360 | NH2 | ARG A | 106 | 7.619 | 39.623 | −5.484 | 1.00 | 100.28 |
| 2361 | C | ARG A | 106 | 1.416 | 41.451 | −2.380 | 1.00 | 64.12 |
| 2362 | O | ARG A | 106 | 0.842 | 40.444 | −2.799 | 1.00 | 64.12 |
| 2363 | N | CYS A | 107 | 1.349 | 42.619 | −3.018 | 1.00 | 99.13 |
| 2364 | CA | CYS A | 107 | 0.651 | 42.685 | −4.301 | 1.00 | 99.13 |
| 2365 | C | CYS A | 107 | 1.710 | 42.307 | −5.317 | 1.00 | 99.13 |
| 2366 | O | CYS A | 107 | 2.639 | 43.059 | −5.575 | 1.00 | 99.13 |
| 2367 | CB | CYS A | 107 | 0.113 | 44.075 | −4.597 | 1.00 | 103.70 |
| 2368 | SG | CYS A | 107 | −1.146 | 44.090 | −5.916 | 1.00 | 103.70 |
| 2369 | N | HIS A | 108 | 1.573 | 41.112 | −5.866 | 1.00 | 72.29 |
| 2370 | CA | HIS A | 108 | 2.530 | 40.575 | −6.804 | 1.00 | 72.29 |
| 2371 | CB | HIS A | 108 | 2.799 | 39.131 | −6.429 | 1.00 | 116.05 |
| 2372 | CG | HIS A | 108 | 3.921 | 38.508 | −7.191 | 1.00 | 116.05 |
| 2373 | CD2 | HIS A | 108 | 3.973 | 37.391 | −7.950 | 1.00 | 116.05 |
| 2374 | ND1 | HIS A | 108 | 5.195 | 39.028 | −7.190 | 1.00 | 116.05 |
| 2375 | CE1 | HIS A | 108 | 5.986 | 38.256 | −7.913 | 1.00 | 116.05 |
| 2376 | NE2 | HIS A | 108 | 5.268 | 37.255 | −8.385 | 1.00 | 116.05 |
| 2377 | C | HIS A | 108 | 2.119 | 40.651 | −8.271 | 1.00 | 72.29 |
| 2378 | O | HIS A | 108 | 1.045 | 40.176 | −8.674 | 1.00 | 72.29 |
| 2379 | N | GLY A | 109 | 2.999 | 41.242 | −9.070 | 1.00 | 118.98 |
| 2380 | CA | GLY A | 109 | 2.735 | 41.367 | −10.485 | 1.00 | 118.98 |
| 2381 | C | GLY A | 109 | 3.202 | 40.136 | −11.231 | 1.00 | 118.98 |
| 2382 | O | GLY A | 109 | 4.083 | 39.410 | −10.772 | 1.00 | 118.98 |
| 2383 | N | TRP A | 110 | 2.603 | 39.894 | −12.389 | 1.00 | 106.09 |
| 2384 | CA | TRP A | 110 | 2.968 | 38.751 | −13.202 | 1.00 | 106.09 |
| 2385 | CB | TRP A | 110 | 2.016 | 38.629 | −14.395 | 1.00 | 134.90 |
| 2386 | CG | TRP A | 110 | 2.418 | 37.581 | −15.361 | 1.00 | 134.90 |
| 2387 | CD2 | TRP A | 110 | 1.980 | 36.223 | −15.380 | 1.00 | 134.90 |
| 2388 | CE2 | TRP A | 110 | 2.657 | 35.576 | −16.431 | 1.00 | 134.90 |
| 2389 | CE3 | TRP A | 110 | 1.072 | 35.481 | −14.604 | 1.00 | 134.90 |
| 2390 | CD1 | TRP A | 110 | 3.314 | 37.707 | −16.370 | 1.00 | 134.90 |
| 2391 | NE1 | TRP A | 110 | 3.466 | 36.509 | −17.021 | 1.00 | 134.90 |
| 2392 | CZ2 | TRP A | 110 | 2.464 | 34.224 | −16.732 | 1.00 | 134.90 |
| 2393 | CZ3 | TRP A | 110 | 0.879 | 34.132 | −14.905 | 1.00 | 134.90 |
| 2394 | CH2 | TRP A | 110 | 1.575 | 33.521 | −15.958 | 1.00 | 134.90 |
| 2395 | C | TRP A | 110 | 4.399 | 38.899 | −13.683 | 1.00 | 106.09 |
| 2396 | O | TRP A | 110 | 4.916 | 40.008 | −13.825 | 1.00 | 106.09 |
| 2397 | N | ARG A | 111 | 5.043 | 37.764 | −13.918 | 1.00 | 87.25 |
| 2398 | CA | ARG A | 111 | 6.426 | 37.750 | −14.392 | 1.00 | 87.25 |
| 2399 | CB | ARG A | 111 | 6.468 | 38.086 | −15.858 | 1.00 | 235.25 |
| 2400 | CG | ARG A | 111 | 6.316 | 36.881 | −16.692 | 1.00 | 235.25 |
| 2401 | CD | ARG A | 111 | 6.642 | 37.245 | −18.072 | 1.00 | 235.25 |
| 2402 | NE | ARG A | 111 | 7.428 | 36.197 | −18.691 | 1.00 | 235.25 |
| 2403 | CZ | ARG A | 111 | 8.674 | 35.887 | −18.358 | 1.00 | 235.25 |
| 2404 | NH1 | ARG A | 111 | 9.295 | 36.552 | −17.392 | 1.00 | 235.25 |
| 2405 | NH2 | ARG A | 111 | 9.290 | 34.895 | −18.988 | 1.00 | 235.25 |
| 2406 | C | ARG A | 111 | 7.358 | 38.697 | −13.665 | 1.00 | 87.25 |
| 2407 | O | ARG A | 111 | 8.402 | 39.105 | −14.191 | 1.00 | 87.25 |
| 2408 | N | ASN A | 112 | 6.964 | 39.048 | −12.453 | 1.00 | 105.23 |
| 2409 | CA | ASN A | 112 | 7.744 | 39.942 | −11.633 | 1.00 | 105.23 |
| 2410 | CB | ASN A | 112 | 9.121 | 39.353 | −11.375 | 1.00 | 116.08 |
| 2411 | CG | ASN A | 112 | 9.735 | 39.907 | −10.118 | 1.00 | 116.08 |
| 2412 | OD1 | ASN A | 112 | 9.369 | 41.000 | −9.660 | 1.00 | 116.08 |
| 2413 | ND2 | ASN A | 112 | 10.668 | 39.166 | −9.544 | 1.00 | 116.08 |
| 2414 | C | ASN A | 112 | 7.905 | 41.345 | −12.218 | 1.00 | 105.23 |
| 2415 | O | ASN A | 112 | 8.852 | 42.055 | −11.885 | 1.00 | 105.23 |
| 2416 | N | TRP A | 113 | 6.992 | 41.753 | −13.089 | 1.00 | 124.66 |
| 2417 | CA | TRP A | 113 | 7.095 | 43.088 | −13.645 | 1.00 | 124.66 |
| 2418 | CB | TRP A | 113 | 6.019 | 43.344 | −14.688 | 1.00 | 167.38 |
| 2419 | CG | TRP A | 113 | 6.315 | 42.730 | −15.979 | 1.00 | 167.38 |
| 2420 | CD2 | TRP A | 113 | 5.379 | 42.134 | −16.868 | 1.00 | 167.38 |
| 2421 | CE2 | TRP A | 113 | 6.091 | 41.718 | −18.006 | 1.00 | 167.38 |
| 2422 | CE3 | TRP A | 113 | 3.997 | 41.914 | −16.816 | 1.00 | 167.38 |
| 2423 | CD1 | TRP A | 113 | 7.533 | 42.658 | −16.592 | 1.00 | 167.38 |
| 2424 | NE1 | TRP A | 113 | 7.406 | 42.049 | −17.813 | 1.00 | 167.38 |
| 2425 | CZ2 | TRP A | 113 | 5.475 | 41.093 | −19.080 | 1.00 | 167.38 |
| 2426 | CZ3 | TRP A | 113 | 3.383 | 41.293 | −17.886 | 1.00 | 167.38 |
| 2427 | CH2 | TRP A | 113 | 4.126 | 40.886 | −19.004 | 1.00 | 167.38 |
| 2428 | C | TRP A | 113 | 6.939 | 44.106 | −12.540 | 1.00 | 124.66 |
| 2429 | O | TRP A | 113 | 6.964 | 43.768 | −11.357 | 1.00 | 124.66 |
| 2430 | N | ASP A | 114 | 6.773 | 45.359 | −12.937 | 1.00 | 183.83 |
| 2431 | CA | ASP A | 114 | 6.603 | 46.430 | −11.981 | 1.00 | 183.83 |
| 2432 | CB | ASP A | 114 | 7.598 | 47.558 | −12.258 | 1.00 | 145.30 |
| 2433 | CG | ASP A | 114 | 8.978 | 47.269 | −11.692 | 1.00 | 145.30 |
| 2434 | OD1 | ASP A | 114 | 9.077 | 47.087 | −10.459 | 1.00 | 145.30 |
| 2435 | OD2 | ASP A | 114 | 9.957 | 47.225 | −12.473 | 1.00 | 145.30 |
| 2436 | C | ASP A | 114 | 5.188 | 46.956 | −12.034 | 1.00 | 183.83 |
| 2437 | O | ASP A | 114 | 4.598 | 47.106 | −13.108 | 1.00 | 183.83 |
| 2438 | N | VAL A | 115 | 4.645 | 47.216 | −10.853 | 1.00 | 117.62 |
| 2439 | CA | VAL A | 115 | 3.294 | 47.740 | −10.735 | 1.00 | 117.62 |
| 2440 | CB | VAL A | 115 | 2.421 | 46.835 | −9.879 | 1.00 | 77.28 |
| 2441 | CG1 | VAL A | 115 | 0.971 | 47.248 | −10.008 | 1.00 | 77.28 |
| 2442 | CG2 | VAL A | 115 | 2.616 | 45.409 | −10.302 | 1.00 | 77.28 |
| 2443 | C | VAL A | 115 | 3.329 | 49.116 | −10.089 | 1.00 | 117.62 |
| 2444 | O | VAL A | 115 | 4.142 | 49.377 | −9.191 | 1.00 | 117.62 |
| 2445 | N | TYR A | 116 | 2.444 | 49.995 | −10.553 | 1.00 | 77.55 |
| 2446 | CA | TYR A | 116 | 2.380 | 51.344 | −10.021 | 1.00 | 77.55 |
| 2447 | CB | TYR A | 116 | 2.831 | 52.352 | −11.086 | 1.00 | 167.00 |
| 2448 | CG | TYR A | 116 | 4.271 | 52.172 | −11.532 | 1.00 | 167.00 |
| 2449 | CD1 | TYR A | 116 | 4.581 | 51.453 | −12.676 | 1.00 | 167.00 |
| 2450 | CE1 | TYR A | 116 | 5.909 | 51.265 | −13.071 | 1.00 | 167.00 |
| 2451 | CD2 | TYR A | 116 | 5.325 | 52.703 | −10.789 | 1.00 | 167.00 |
| 2452 | CE2 | TYR A | 116 | 6.653 | 52.519 | −11.173 | 1.00 | 167.00 |
| 2453 | CZ | TYR A | 116 | 6.937 | 51.800 | −12.312 | 1.00 | 167.00 |
| 2454 | OH | TYR A | 116 | 8.246 | 51.606 | −12.687 | 1.00 | 167.00 |
| 2455 | C | TYR A | 116 | 0.984 | 51.699 | −9.519 | 1.00 | 77.55 |
| 2456 | O | TYR A | 116 | 0.023 | 50.951 | −9.742 | 1.00 | 77.55 |
| 2457 | N | LYS A | 117 | 0.879 | 52.842 | −8.840 | 1.00 | 94.85 |
| 2458 | CA | LYS A | 117 | −0.399 | 53.292 | −8.310 | 1.00 | 94.85 |
| 2459 | CB | LYS A | 117 | −1.300 | 53.834 | −9.423 | 1.00 | 193.46 |
| 2460 | CG | LYS A | 117 | −1.084 | 55.291 | −9.786 | 1.00 | 193.46 |
| 2461 | CD | LYS A | 117 | −2.284 | 55.824 | −10.563 | 1.00 | 193.46 |
| 2462 | CE | LYS A | 117 | −3.569 | 55.726 | −9.735 | 1.00 | 193.46 |
| 2463 | NZ | LYS A | 117 | −4.780 | 56.210 | −10.464 | 1.00 | 193.46 |
| 2464 | C | LYS A | 117 | −1.099 | 52.125 | −7.629 | 1.00 | 94.85 |
| 2465 | O | LYS A | 117 | −2.226 | 51.770 | −7.977 | 1.00 | 94.85 |
| 2466 | N | VAL A | 118 | −0.422 | 51.530 | −6.655 | 1.00 | 105.41 |
| 2467 | CA | VAL A | 118 | −0.979 | 50.402 | −5.927 | 1.00 | 105.41 |
| 2468 | CB | VAL A | 118 | 0.122 | 49.503 | −5.445 | 1.00 | 73.04 |
| 2469 | CG1 | VAL A | 118 | −0.314 | 48.777 | −4.205 | 1.00 | 73.04 |
| 2470 | CG2 | VAL A | 118 | 0.455 | 48.521 | −6.514 | 1.00 | 73.04 |
| 2471 | C | VAL A | 118 | −1.862 | 50.736 | −4.723 | 1.00 | 105.41 |
| 2472 | O | VAL A | 118 | −1.527 | 51.582 | −3.894 | 1.00 | 105.41 |
| 2473 | N | ILE A | 119 | −2.971 | 50.020 | −4.607 | 1.00 | 71.97 |
| 2474 | CA | ILE A | 119 | −3.902 | 50.248 | −3.518 | 1.00 | 71.97 |
| 2475 | CB | ILE A | 119 | −5.125 | 51.002 | −4.016 | 1.00 | 77.41 |
| 2476 | CG2 | ILE A | 119 | −6.037 | 51.319 | −2.866 | 1.00 | 77.41 |
| 2477 | CG1 | ILE A | 119 | −4.687 | 52.285 | −4.705 | 1.00 | 77.41 |
| 2478 | CD1 | ILE A | 119 | −5.804 | 52.949 | −5.467 | 1.00 | 77.41 |
| 2479 | C | ILE A | 119 | −4.395 | 48.928 | −2.961 | 1.00 | 71.97 |
| 2480 | O | ILE A | 119 | −4.954 | 48.146 | −3.701 | 1.00 | 71.97 |
| 2481 | N | TYR A | 120 | −4.193 | 48.654 | −1.679 | 1.00 | 64.29 |
| 2482 | CA | TYR A | 120 | −4.698 | 47.403 | −1.117 | 1.00 | 64.29 |
| 2483 | CB | TYR A | 120 | −3.867 | 46.908 | 0.059 | 1.00 | 49.60 |
| 2484 | CG | TYR A | 120 | −2.521 | 46.438 | −0.297 | 1.00 | 49.60 |
| 2485 | CD1 | TYR A | 120 | −1.472 | 47.324 | −0.395 | 1.00 | 49.60 |
| 2486 | CE1 | TYR A | 120 | −0.195 | 46.897 | −0.736 | 1.00 | 49.60 |
| 2487 | CD2 | TYR A | 120 | −2.292 | 45.109 | −0.546 | 1.00 | 49.60 |
| 2488 | CE2 | TYR A | 120 | −1.026 | 44.650 | −0.901 | 1.00 | 49.60 |
| 2489 | CZ | TYR A | 120 | 0.020 | 45.548 | −0.992 | 1.00 | 49.60 |
| 2490 | OH | TYR A | 120 | 1.268 | 45.095 | −1.339 | 1.00 | 49.60 |
| 2491 | C | TYR A | 120 | −6.069 | 47.679 | −0.580 | 1.00 | 64.29 |
| 2492 | O | TYR A | 120 | −6.313 | 48.764 | −0.069 | 1.00 | 64.29 |
| 2493 | N | TYR A | 121 | −6.945 | 46.686 | −0.658 | 1.00 | 62.29 |
| 2494 | CA | TYR A | 121 | −8.299 | 46.838 | −0.154 | 1.00 | 62.29 |
| 2495 | CB | TYR A | 121 | −9.315 | 46.752 | −1.302 | 1.00 | 87.89 |
| 2496 | CG | TYR A | 121 | −9.308 | 47.900 | −2.293 | 1.00 | 87.89 |
| 2497 | CD1 | TYR A | 121 | −8.219 | 48.119 | −3.126 | 1.00 | 87.89 |
| 2498 | CE1 | TYR A | 121 | −8.232 | 49.147 | −4.073 | 1.00 | 87.89 |
| 2499 | CD2 | TYR A | 121 | −10.422 | 48.745 | −2.424 | 1.00 | 87.89 |
| 2500 | CE2 | TYR A | 121 | −10.450 | 49.776 | −3.368 | 1.00 | 87.89 |
| 2501 | CZ | TYR A | 121 | −9.351 | 49.970 | −4.193 | 1.00 | 87.89 |
| 2502 | OH | TYR A | 121 | −9.383 | 50.966 | −5.156 | 1.00 | 87.89 |
| 2503 | C | TYR A | 121 | −8.647 | 45.772 | 0.883 | 1.00 | 62.29 |
| 2504 | O | TYR A | 121 | −8.275 | 44.598 | 0.723 | 1.00 | 62.29 |
| 2505 | N | LYS A | 122 | −9.349 | 46.180 | 1.943 | 1.00 | 53.98 |
| 2506 | CA | LYS A | 122 | −9.794 | 45.238 | 2.957 | 1.00 | 53.98 |
| 2507 | CB | LYS A | 122 | −9.069 | 45.436 | 4.278 | 1.00 | 98.53 |
| 2508 | CG | LYS A | 122 | −9.499 | 44.427 | 5.329 | 1.00 | 98.53 |
| 2509 | CD | LYS A | 122 | −9.038 | 44.809 | 6.719 | 1.00 | 98.53 |
| 2510 | CE | LYS A | 122 | −9.644 | 43.912 | 7.774 | 1.00 | 98.53 |
| 2511 | NZ | LYS A | 122 | −9.317 | 44.426 | 9.120 | 1.00 | 98.53 |
| 2512 | C | LYS A | 122 | −11.291 | 45.452 | 3.158 | 1.00 | 53.98 |
| 2513 | O | LYS A | 122 | −11.720 | 46.526 | 3.569 | 1.00 | 53.98 |
| 2514 | N | ASP A | 123 | −12.081 | 44.429 | 2.841 | 1.00 | 82.84 |
| 2515 | CA | ASP A | 123 | −13.530 | 44.491 | 2.976 | 1.00 | 82.84 |
| 2516 | CB | ASP A | 123 | −13.926 | 44.624 | 4.449 | 1.00 | 104.85 |
| 2517 | CG | ASP A | 123 | −13.786 | 43.313 | 5.204 | 1.00 | 104.85 |
| 2518 | OD1 | ASP A | 123 | −14.244 | 42.269 | 4.680 | 1.00 | 104.85 |
| 2519 | OD2 | ASP A | 123 | −13.228 | 43.321 | 6.324 | 1.00 | 104.85 |
| 2520 | C | ASP A | 123 | −14.140 | 45.620 | 2.158 | 1.00 | 82.84 |
| 2521 | O | ASP A | 123 | −15.013 | 46.350 | 2.638 | 1.00 | 82.84 |
| 2522 | N | GLY A | 124 | −13.677 | 45.743 | 0.915 | 1.00 | 89.57 |
| 2523 | CA | GLY A | 124 | −14.179 | 46.772 | 0.018 | 1.00 | 89.57 |
| 2524 | C | GLY A | 124 | −13.699 | 48.197 | 0.279 | 1.00 | 89.57 |
| 2525 | O | GLY A | 124 | −13.981 | 49.093 | −0.528 | 1.00 | 89.57 |
| 2526 | N | GLU A | 125 | −12.978 | 48.412 | 1.382 | 1.00 | 81.16 |
| 2527 | CA | GLU A | 125 | −12.476 | 49.746 | 1.745 | 1.00 | 81.16 |
| 2528 | CB | GLU A | 125 | −12.470 | 49.925 | 3.274 | 1.00 | 176.94 |
| 2529 | CG | GLU A | 125 | −13.834 | 49.988 | 3.958 | 1.00 | 176.94 |
| 2530 | CD | GLU A | 125 | −14.499 | 51.350 | 3.844 | 1.00 | 176.94 |
| 2531 | OE1 | GLU A | 125 | −13.931 | 52.343 | 4.352 | 1.00 | 176.94 |
| 2532 | OE2 | GLU A | 125 | −15.595 | 51.428 | 3.251 | 1.00 | 176.94 |
| 2533 | C | GLU A | 125 | −11.055 | 50.008 | 1.238 | 1.00 | 81.16 |
| 2534 | O | GLU A | 125 | −10.223 | 49.116 | 1.229 | 1.00 | 81.16 |
| 2535 | N | ALA A | 126 | −10.772 | 51.228 | 0.815 | 1.00 | 92.74 |
| 2536 | CA | ALA A | 126 | −9.424 | 51.546 | 0.375 | 1.00 | 92.74 |
| 2537 | CB | ALA A | 126 | −9.379 | 52.967 | −0.145 | 1.00 | 165.28 |
| 2538 | C | ALA A | 126 | −8.592 | 51.410 | 1.650 | 1.00 | 92.74 |
| 2539 | O | ALA A | 126 | −9.083 | 51.719 | 2.731 | 1.00 | 92.74 |
| 2540 | N | LEU A | 127 | −7.347 | 50.957 | 1.550 | 1.00 | 58.95 |
| 2541 | CA | LEU A | 127 | −6.544 | 50.778 | 2.749 | 1.00 | 58.95 |
| 2542 | CB | LEU A | 127 | −6.333 | 49.305 | 3.037 | 1.00 | 73.14 |
| 2543 | CG | LEU A | 127 | −6.046 | 49.150 | 4.528 | 1.00 | 73.14 |
| 2544 | CD1 | LEU A | 127 | −7.224 | 49.745 | 5.285 | 1.00 | 73.14 |
| 2545 | CD2 | LEU A | 127 | −5.840 | 47.693 | 4.917 | 1.00 | 73.14 |
| 2546 | C | LEU A | 127 | −5.195 | 51.457 | 2.764 | 1.00 | 58.95 |
| 2547 | O | LEU A | 127 | −4.910 | 52.212 | 3.691 | 1.00 | 58.95 |
| 2548 | N | LYS A | 128 | −4.344 | 51.153 | 1.788 | 1.00 | 77.17 |
| 2549 | CA | LYS A | 128 | −3.028 | 51.788 | 1.690 | 1.00 | 77.17 |
| 2550 | CB | LYS A | 128 | −1.920 | 50.862 | 2.197 | 1.00 | 133.78 |
| 2551 | CG | LYS A | 128 | −2.041 | 50.465 | 3.656 | 1.00 | 133.78 |
| 2552 | CD | LYS A | 128 | −1.716 | 51.601 | 4.605 | 1.00 | 133.78 |
| 2553 | CE | LYS A | 128 | −1.741 | 51.120 | 6.052 | 1.00 | 133.78 |
| 2554 | NZ | LYS A | 128 | −1.293 | 52.157 | 7.033 | 1.00 | 133.78 |
| 2555 | C | LYS A | 128 | −2.788 | 52.130 | 0.212 | 1.00 | 77.17 |
| 2556 | O | LYS A | 128 | −3.348 | 51.493 | −0.675 | 1.00 | 77.17 |
| 2557 | N | TYR A | 129 | −1.973 | 53.142 | −0.063 | 1.00 | 64.91 |
| 2558 | CA | TYR A | 129 | −1.693 | 53.519 | −1.444 | 1.00 | 64.91 |
| 2559 | CB | TYR A | 129 | −2.633 | 54.637 | −1.882 | 1.00 | 122.39 |
| 2560 | CG | TYR A | 129 | −2.100 | 55.390 | −3.080 | 1.00 | 122.39 |
| 2561 | CD1 | TYR A | 129 | −2.232 | 54.874 | −4.366 | 1.00 | 122.39 |
| 2562 | CE1 | TYR A | 129 | −1.702 | 55.539 | −5.465 | 1.00 | 122.39 |
| 2563 | CD2 | TYR A | 129 | −1.416 | 56.599 | −2.919 | 1.00 | 122.39 |
| 2564 | CE2 | TYR A | 129 | −0.875 | 57.273 | −4.012 | 1.00 | 122.39 |
| 2565 | CZ | TYR A | 129 | −1.024 | 56.738 | −5.282 | 1.00 | 122.39 |
| 2566 | OH | TYR A | 129 | −0.508 | 57.402 | −6.370 | 1.00 | 122.39 |
| 2567 | C | TYR A | 129 | −0.244 | 53.978 | −1.679 | 1.00 | 64.91 |
| 2568 | O | TYR A | 129 | 0.320 | 54.739 | −0.885 | 1.00 | 64.91 |
| 2569 | N | TRP A | 130 | 0.348 | 53.530 | −2.787 | 1.00 | 121.28 |
| 2570 | CA | TRP A | 130 | 1.713 | 53.914 | −3.125 | 1.00 | 121.28 |
| 2571 | CB | TRP A | 130 | 2.715 | 52.874 | −2.627 | 1.00 | 194.88 |
| 2572 | CG | TRP A | 130 | 2.557 | 52.464 | −1.196 | 1.00 | 194.88 |
| 2573 | CD2 | TRP A | 130 | 3.398 | 52.848 | −0.100 | 1.00 | 194.88 |
| 2574 | CE2 | TRP A | 130 | 2.909 | 52.182 | 1.049 | 1.00 | 194.88 |
| 2575 | CE3 | TRP A | 130 | 4.508 | 53.694 | 0.023 | 1.00 | 194.88 |
| 2576 | CD1 | TRP A | 130 | 1.629 | 51.608 | −0.683 | 1.00 | 194.88 |
| 2577 | NE1 | TRP A | 130 | 1.833 | 51.431 | 0.666 | 1.00 | 194.88 |
| 2578 | CZ2 | TRP A | 130 | 3.500 | 52.334 | 2.309 | 1.00 | 194.88 |
| 2579 | CZ3 | TRP A | 130 | 5.096 | 53.847 | 1.280 | 1.00 | 194.88 |
| 2580 | CH2 | TRP A | 130 | 4.592 | 53.163 | 2.403 | 1.00 | 194.88 |
| 2581 | C | TRP A | 130 | 1.907 | 54.064 | −4.627 | 1.00 | 121.28 |
| 2582 | O | TRP A | 130 | 1.075 | 53.627 | −5.422 | 1.00 | 121.28 |
| 2583 | N | TYR A | 131 | 3.015 | 54.685 | −5.015 | 1.00 | 100.84 |
| 2584 | CA | TYR A | 131 | 3.304 | 54.849 | −6.426 | 1.00 | 100.84 |
| 2585 | CB | TYR A | 131 | 4.202 | 56.059 | −6.683 | 1.00 | 199.69 |
| 2586 | CG | TYR A | 131 | 4.299 | 56.369 | −8.155 | 1.00 | 199.69 |
| 2587 | CD1 | TYR A | 131 | 3.223 | 56.944 | −8.830 | 1.00 | 199.69 |
| 2588 | CE1 | TYR A | 131 | 3.246 | 57.115 | −10.206 | 1.00 | 199.69 |
| 2589 | CD2 | TYR A | 131 | 5.414 | 55.982 | −8.899 | 1.00 | 199.69 |
| 2590 | CE2 | TYR A | 131 | 5.448 | 56.148 | −10.281 | 1.00 | 199.69 |
| 2591 | CZ | TYR A | 131 | 4.357 | 56.712 | −10.926 | 1.00 | 199.69 |
| 2592 | OH | TYR A | 131 | 4.364 | 56.843 | −12.295 | 1.00 | 199.69 |
| 2593 | C | TYR A | 131 | 4.029 | 53.572 | −6.818 | 1.00 | 100.84 |
| 2594 | O | TYR A | 131 | 3.397 | 52.644 | −7.326 | 1.00 | 100.84 |
| 2595 | N | GLU A | 132 | 5.351 | 53.543 | −6.624 | 1.00 | 218.16 |
| 2596 | CA | GLU A | 132 | 6.122 | 52.331 | −6.894 | 1.00 | 218.16 |
| 2597 | CB | GLU A | 132 | 7.629 | 52.547 | −6.666 | 1.00 | 249.55 |
| 2598 | CG | GLU A | 132 | 8.382 | 53.317 | −7.762 | 1.00 | 249.55 |
| 2599 | CD | GLU A | 132 | 9.480 | 52.482 | −8.422 | 1.00 | 249.55 |
| 2600 | OE1 | GLU A | 132 | 9.903 | 51.469 | −7.822 | 1.00 | 249.55 |
| 2601 | OE2 | GLU A | 132 | 9.922 | 52.848 | −9.533 | 1.00 | 249.55 |
| 2602 | C | GLU A | 132 | 5.531 | 51.536 | −5.747 | 1.00 | 218.16 |
| 2603 | O | GLU A | 132 | 5.514 | 52.022 | −4.616 | 1.00 | 218.16 |
| 2604 | N | ASN A | 133 | 5.032 | 50.334 | −6.012 | 1.00 | 155.99 |
| 2605 | CA | ASN A | 133 | 4.388 | 49.588 | −4.939 | 1.00 | 155.99 |
| 2606 | CB | ASN A | 133 | 3.656 | 48.353 | −5.472 | 1.00 | 108.04 |
| 2607 | CG | ASN A | 133 | 4.538 | 47.157 | −5.575 | 1.00 | 108.04 |
| 2608 | OD1 | ASN A | 133 | 5.620 | 47.230 | −6.143 | 1.00 | 108.04 |
| 2609 | ND2 | ASN A | 133 | 4.085 | 46.035 | −5.035 | 1.00 | 108.04 |
| 2610 | C | ASN A | 133 | 5.244 | 49.194 | −3.759 | 1.00 | 155.99 |
| 2611 | O | ASN A | 133 | 6.458 | 49.376 | −3.734 | 1.00 | 155.99 |
| 2612 | N | HIS A | 134 | 4.560 | 48.629 | −2.779 | 1.00 | 115.35 |
| 2613 | CA | HIS A | 134 | 5.153 | 48.235 | −1.520 | 1.00 | 115.35 |
| 2614 | CB | HIS A | 134 | 4.782 | 49.305 | −0.489 | 1.00 | 200.02 |
| 2615 | CG | HIS A | 134 | 5.436 | 49.129 | 0.842 | 1.00 | 200.02 |
| 2616 | CD2 | HIS A | 134 | 4.912 | 48.893 | 2.067 | 1.00 | 200.02 |
| 2617 | ND1 | HIS A | 134 | 6.799 | 49.213 | 1.016 | 1.00 | 200.02 |
| 2618 | CE1 | HIS A | 134 | 7.088 | 49.035 | 2.293 | 1.00 | 200.02 |
| 2619 | NE2 | HIS A | 134 | 5.961 | 48.840 | 2.951 | 1.00 | 200.02 |
| 2620 | C | HIS A | 134 | 4.596 | 46.874 | −1.114 | 1.00 | 115.35 |
| 2621 | O | HIS A | 134 | 4.008 | 46.161 | −1.934 | 1.00 | 115.35 |
| 2622 | N | ASN A | 135 | 4.781 | 46.524 | 0.153 | 1.00 | 81.38 |
| 2623 | CA | ASN A | 135 | 4.298 | 45.263 | 0.675 | 1.00 | 81.38 |
| 2624 | CB | ASN A | 135 | 5.426 | 44.243 | 0.654 | 1.00 | 168.37 |
| 2625 | CG | ASN A | 135 | 5.832 | 43.891 | −0.754 | 1.00 | 168.37 |
| 2626 | OD1 | ASN A | 135 | 4.964 | 43.656 | −1.596 | 1.00 | 168.37 |
| 2627 | ND2 | ASN A | 135 | 7.134 | 43.839 | −1.029 | 1.00 | 168.37 |
| 2628 | C | ASN A | 135 | 3.748 | 45.431 | 2.073 | 1.00 | 81.38 |
| 2629 | O | ASN A | 135 | 4.455 | 45.219 | 3.042 | 1.00 | 81.38 |
| 2630 | N | ILE A | 136 | 2.481 | 45.817 | 2.168 | 1.00 | 68.07 |
| 2631 | CA | ILE A | 136 | 1.826 | 46.032 | 3.456 | 1.00 | 68.07 |
| 2632 | CB | ILE A | 136 | 0.288 | 46.019 | 3.287 | 1.00 | 86.88 |
| 2633 | CG2 | ILE A | 136 | −0.135 | 44.814 | 2.531 | 1.00 | 86.88 |
| 2634 | CG1 | ILE A | 136 | −0.397 | 46.040 | 4.638 | 1.00 | 86.88 |
| 2635 | CD1 | ILE A | 136 | −1.885 | 46.136 | 4.514 | 1.00 | 86.88 |
| 2636 | C | ILE A | 136 | 2.277 | 44.997 | 4.482 | 1.00 | 68.07 |
| 2637 | O | ILE A | 136 | 2.085 | 43.801 | 4.301 | 1.00 | 68.07 |
| 2638 | N | SER A | 137 | 2.904 | 45.484 | 5.550 | 1.00 | 113.35 |
| 2639 | CA | SER A | 137 | 3.422 | 44.631 | 6.606 | 1.00 | 113.35 |
| 2640 | CB | SER A | 137 | 4.932 | 44.798 | 6.686 | 1.00 | 73.04 |
| 2641 | OG | SER A | 137 | 5.433 | 44.258 | 7.891 | 1.00 | 73.04 |
| 2642 | C | SER A | 137 | 2.808 | 44.903 | 7.974 | 1.00 | 113.35 |
| 2643 | O | SER A | 137 | 2.469 | 46.029 | 8.304 | 1.00 | 113.35 |
| 2644 | N | ILE A | 138 | 2.688 | 43.856 | 8.777 | 1.00 | 71.19 |
| 2645 | CA | ILE A | 138 | 2.116 | 43.961 | 10.117 | 1.00 | 71.19 |
| 2646 | CB | ILE A | 138 | 0.715 | 43.413 | 10.147 | 1.00 | 41.44 |
| 2647 | CG2 | ILE A | 138 | 0.257 | 43.304 | 11.582 | 1.00 | 41.44 |
| 2648 | CG1 | ILE A | 138 | −0.212 | 44.297 | 9.318 | 1.00 | 41.44 |
| 2649 | CD1 | ILE A | 138 | 1.531 | 43.627 | 9.019 | 1.00 | 41.44 |
| 2650 | C | ILE A | 138 | 2.922 | 43.170 | 11.146 | 1.00 | 71.19 |
| 2651 | O | ILE A | 138 | 3.093 | 41.954 | 11.012 | 1.00 | 71.19 |
| 2652 | N | THR A | 139 | 3.397 | 43.856 | 12.185 | 1.00 | 108.53 |
| 2653 | CA | THR A | 139 | 4.195 | 43.216 | 13.234 | 1.00 | 108.53 |
| 2654 | CB | THR A | 139 | 5.001 | 44.260 | 14.012 | 1.00 | 232.49 |
| 2655 | OG1 | THR A | 139 | 4.127 | 45.304 | 14.460 | 1.00 | 232.49 |
| 2656 | CG2 | THR A | 139 | 6.080 | 44.854 | 13.121 | 1.00 | 232.49 |
| 2657 | C | THR A | 139 | 3.291 | 42.456 | 14.192 | 1.00 | 108.53 |
| 2658 | O | THR A | 139 | 3.199 | 41.235 | 14.125 | 1.00 | 108.53 |
| 2659 | N | ASN A | 140 | 2.632 | 43.192 | 15.083 | 1.00 | 125.42 |
| 2660 | CA | ASN A | 140 | 1.699 | 42.621 | 16.050 | 1.00 | 125.42 |
| 2661 | CB | ASN A | 140 | 1.662 | 43.455 | 17.328 | 1.00 | 148.98 |
| 2662 | CG | ASN A | 140 | 0.619 | 42.967 | 18.305 | 1.00 | 148.98 |
| 2663 | OD1 | ASN A | 140 | −0.533 | 42.738 | 17.950 | 1.00 | 148.98 |
| 2664 | ND2 | ASN A | 140 | 1.024 | 42.828 | 19.558 | 1.00 | 148.98 |
| 2665 | C | ASN A | 140 | 0.335 | 42.677 | 15.375 | 1.00 | 125.42 |
| 2666 | O | ASN A | 140 | −0.149 | 43.763 | 15.030 | 1.00 | 125.42 |
| 2667 | N | ALA A | 141 | −0.291 | 41.518 | 15.203 | 1.00 | 57.61 |
| 2668 | CA | ALA A | 141 | −1.569 | 41.462 | 14.527 | 1.00 | 57.61 |
| 2669 | CB | ALA A | 141 | −1.605 | 40.246 | 13.644 | 1.00 | 27.12 |
| 2670 | C | ALA A | 141 | −2.785 | 41.468 | 15.439 | 1.00 | 57.61 |
| 2671 | O | ALA A | 141 | −2.895 | 40.661 | 16.364 | 1.00 | 57.61 |
| 2672 | N | THR A | 142 | −3.713 | 42.373 | 15.149 | 1.00 | 70.60 |
| 2673 | CA | THR A | 142 | −4.939 | 42.481 | 15.912 | 1.00 | 70.60 |
| 2674 | CB | THR A | 142 | −5.488 | 43.908 | 15.811 | 1.00 | 136.27 |
| 2675 | OG1 | THR A | 142 | −4.440 | 44.833 | 16.136 | 1.00 | 136.27 |
| 2676 | CG2 | THR A | 142 | −6.643 | 44.104 | 16.773 | 1.00 | 136.27 |
| 2677 | C | THR A | 142 | −5.937 | 41.478 | 15.334 | 1.00 | 70.60 |
| 2678 | O | THR A | 142 | −5.666 | 40.852 | 14.311 | 1.00 | 70.60 |
| 2679 | N | VAL A | 143 | −7.066 | 41.285 | 16.001 | 1.00 | 71.67 |
| 2680 | CA | VAL A | 143 | −8.057 | 40.355 | 15.489 | 1.00 | 71.67 |
| 2681 | CB | VAL A | 143 | −8.949 | 39.782 | 16.610 | 1.00 | 65.94 |
| 2682 | CG1 | VAL A | 143 | −9.785 | 40.880 | 17.217 | 1.00 | 65.94 |
| 2683 | CG2 | VAL A | 143 | −9.848 | 38.672 | 16.047 | 1.00 | 65.94 |
| 2684 | C | VAL A | 143 | −8.934 | 41.126 | 14.518 | 1.00 | 71.67 |
| 2685 | O | VAL A | 143 | −9.679 | 40.552 | 13.726 | 1.00 | 71.67 |
| 2686 | N | GLU A | 144 | −8.842 | 42.442 | 14.579 | 1.00 | 71.12 |
| 2687 | CA | GLU A | 144 | −9.650 | 43.260 | 13.699 | 1.00 | 71.12 |
| 2688 | CB | GLU A | 144 | −9.747 | 44.691 | 14.235 | 1.00 | 228.43 |
| 2689 | CG | GLU A | 144 | −10.475 | 44.796 | 15.566 | 1.00 | 228.43 |
| 2690 | CD | GLU A | 144 | −9.558 | 45.204 | 16.699 | 1.00 | 228.43 |
| 2691 | OE1 | GLU A | 144 | −8.966 | 46.296 | 16.611 | 1.00 | 228.43 |
| 2692 | OE2 | GLU A | 144 | −9.428 | 44.440 | 17.676 | 1.00 | 228.43 |
| 2693 | C | GLU A | 144 | −9.068 | 43.250 | 12.301 | 1.00 | 71.12 |
| 2694 | O | GLU A | 144 | −9.732 | 43.609 | 11.338 | 1.00 | 71.12 |
| 2695 | N | ASP A | 145 | −7.821 | 42.820 | 12.194 | 1.00 | 58.24 |
| 2696 | CA | ASP A | 145 | −7.146 | 42.754 | 10.900 | 1.00 | 58.24 |
| 2697 | CB | ASP A | 145 | −5.645 | 42.541 | 11.091 | 1.00 | 106.20 |
| 2698 | CG | ASP A | 145 | −4.945 | 43.784 | 11.606 | 1.00 | 106.20 |
| 2699 | OD1 | ASP A | 145 | −5.013 | 44.817 | 10.911 | 1.00 | 106.20 |
| 2700 | OD2 | ASP A | 145 | −4.329 | 43.733 | 12.696 | 1.00 | 106.20 |
| 2701 | C | ASP A | 145 | −7.705 | 41.643 | 10.018 | 1.00 | 58.24 |
| 2702 | O | ASP A | 145 | −7.434 | 41.608 | 8.819 | 1.00 | 58.24 |
| 2703 | N | SER A | 146 | −8.490 | 40.744 | 10.607 | 1.00 | 85.01 |
| 2704 | CA | SER A | 146 | −9.077 | 39.652 | 9.848 | 1.00 | 85.01 |
| 2705 | CB | SER A | 146 | −9.781 | 38.669 | 10.789 | 1.00 | 118.46 |
| 2706 | OG | SER A | 146 | −8.854 | 38.089 | 11.691 | 1.00 | 118.46 |
| 2707 | C | SER A | 146 | −10.052 | 40.266 | 8.855 | 1.00 | 85.01 |
| 2708 | O | SER A | 146 | −10.741 | 41.227 | 9.168 | 1.00 | 85.01 |
| 2709 | N | GLY A | 147 | −10.081 | 39.735 | 7.644 | 1.00 | 64.55 |
| 2710 | CA | GLY A | 147 | −10.972 | 40.264 | 6.632 | 1.00 | 64.55 |
| 2711 | C | GLY A | 147 | −10.649 | 39.664 | 5.277 | 1.00 | 64.55 |
| 2712 | O | GLY A | 147 | −9.963 | 38.628 | 5.214 | 1.00 | 64.55 |
| 2713 | N | THR A | 148 | −11.147 | 40.285 | 4.201 | 1.00 | 54.60 |
| 2714 | CA | THR A | 148 | −10.881 | 39.795 | 2.841 | 1.00 | 54.60 |
| 2715 | CB | THR A | 148 | −12.159 | 39.339 | 2.143 | 1.00 | 77.82 |
| 2716 | OG1 | THR A | 148 | −12.541 | 40.316 | 1.193 | 1.00 | 77.82 |
| 2717 | CG2 | THR A | 148 | −13.272 | 39.179 | 3.148 | 1.00 | 77.82 |
| 2718 | C | THR A | 148 | −10.204 | 40.891 | 2.029 | 1.00 | 54.60 |
| 2719 | O | THR A | 148 | −10.789 | 41.941 | 1.746 | 1.00 | 54.60 |
| 2720 | N | TYR A | 149 | −8.958 | 40.639 | 1.661 | 1.00 | 38.49 |
| 2721 | CA | TYR A | 149 | −8.181 | 41.622 | 0.950 | 1.00 | 38.49 |
| 2722 | CB | TYR A | 149 | −6.775 | 41.604 | 1.518 | 1.00 | 47.71 |
| 2723 | CG | TYR A | 149 | −6.654 | 41.954 | 2.987 | 1.00 | 47.71 |
| 2724 | CD1 | TYR A | 149 | −7.128 | 41.123 | 3.982 | 1.00 | 47.71 |
| 2725 | CE1 | TYR A | 149 | −6.952 | 41.468 | 5.327 | 1.00 | 47.71 |
| 2726 | CD2 | TYR A | 149 | −6.010 | 43.124 | 3.370 | 1.00 | 47.71 |
| 2727 | CE2 | TYR A | 149 | −5.832 | 43.470 | 4.691 | 1.00 | 47.71 |
| 2728 | CZ | TYR A | 149 | −6.297 | 42.656 | 5.669 | 1.00 | 47.71 |
| 2729 | OH | TYR A | 149 | −6.098 | 43.066 | 6.973 | 1.00 | 47.71 |
| 2730 | C | TYR A | 149 | −8.098 | 41.368 | −0.543 | 1.00 | 38.49 |
| 2731 | O | TYR A | 149 | −8.451 | 40.272 | −1.006 | 1.00 | 38.49 |
| 2732 | N | TYR A | 150 | −7.639 | 42.382 | −1.279 | 1.00 | 53.38 |
| 2733 | CA | TYR A | 150 | −7.385 | 42.305 | −2.716 | 1.00 | 53.38 |
| 2734 | CB | TYR A | 150 | −8.681 | 42.142 | −3.520 | 1.00 | 86.43 |
| 2735 | CG | TYR A | 150 | −9.564 | 43.353 | −3.735 | 1.00 | 86.43 |
| 2736 | CD1 | TYR A | 150 | −9.167 | 44.393 | −4.563 | 1.00 | 86.43 |
| 2737 | CE1 | TYR A | 150 | −9.992 | 45.488 | −4.795 | 1.00 | 86.43 |
| 2738 | CD2 | TYR A | 150 | −10.816 | 43.436 | −3.142 | 1.00 | 86.43 |
| 2739 | CE2 | TYR A | 150 | −11.652 | 44.522 | −3.365 | 1.00 | 86.43 |
| 2740 | CZ | TYR A | 150 | −11.234 | 45.547 | −4.190 | 1.00 | 86.43 |
| 2741 | OH | TYR A | 150 | −12.049 | 46.642 | −4.381 | 1.00 | 86.43 |
| 2742 | C | TYR A | 150 | −6.653 | 43.598 | −3.028 | 1.00 | 53.38 |
| 2743 | O | TYR A | 150 | −6.726 | 44.536 | −2.225 | 1.00 | 53.38 |
| 2744 | N | CYS A | 151 | −5.900 | 43.660 | −4.127 | 1.00 | 73.39 |
| 2745 | CA | CYS A | 151 | −5.179 | 44.894 | −4.462 | 1.00 | 73.39 |
| 2746 | C | CYS A | 151 | −5.388 | 45.311 | −5.900 | 1.00 | 73.39 |
| 2747 | O | CYS A | 151 | −5.741 | 44.487 | −6.721 | 1.00 | 73.39 |
| 2748 | CB | CYS A | 151 | −3.680 | 44.745 | −4.197 | 1.00 | 73.27 |
| 2749 | SG | CYS A | 151 | −2.861 | 43.358 | −5.059 | 1.00 | 73.27 |
| 2750 | N | THR A | 152 | −5.177 | 46.593 | −6.192 | 1.00 | 98.75 |
| 2751 | CA | THR A | 152 | −5.335 | 47.121 | −7.537 | 1.00 | 98.75 |
| 2752 | CB | THR A | 152 | −6.478 | 48.154 | −7.602 | 1.00 | 109.76 |
| 2753 | OG1 | THR A | 152 | −6.138 | 49.310 | −6.821 | 1.00 | 109.76 |
| 2754 | CG2 | THR A | 152 | −7.746 | 47.558 | −7.048 | 1.00 | 109.76 |
| 2755 | C | THR A | 152 | −4.025 | 47.793 | −7.905 | 1.00 | 98.75 |
| 2756 | O | THR A | 152 | −3.329 | 48.322 | −7.032 | 1.00 | 98.75 |
| 2757 | N | GLY A | 153 | −3.681 | 47.764 | −9.188 | 1.00 | 91.16 |
| 2758 | CA | GLY A | 153 | −2.444 | 48.385 | −9.622 | 1.00 | 91.16 |
| 2759 | C | GLY A | 153 | −2.392 | 48.562 | −11.122 | 1.00 | 91.16 |
| 2760 | O | GLY A | 153 | −3.163 | 47.947 | −11.843 | 1.00 | 91.16 |
| 2761 | N | LYS A | 154 | −1.488 | 49.409 | −11.597 | 1.00 | 71.11 |
| 2762 | CA | LYS A | 154 | −1.359 | 49.643 | −13.023 | 1.00 | 71.11 |
| 2763 | CB | LYS A | 154 | −1.229 | 51.140 | −13.299 | 1.00 | 173.07 |
| 2764 | CG | LYS A | 154 | −1.235 | 51.523 | −14.769 | 1.00 | 173.07 |
| 2765 | CD | LYS A | 154 | −1.155 | 53.036 | −14.911 | 1.00 | 173.07 |
| 2766 | CE | LYS A | 154 | −1.050 | 53.490 | −16.359 | 1.00 | 173.07 |
| 2767 | NZ | LYS A | 154 | −0.857 | 54.960 | −16.420 | 1.00 | 173.07 |
| 2768 | C | LYS A | 154 | −0.120 | 48.907 | −13.500 | 1.00 | 71.11 |
| 2769 | O | LYS A | 154 | 0.963 | 49.009 | −12.900 | 1.00 | 71.11 |
| 2770 | N | VAL A | 155 | −0.289 | 48.128 | −14.563 | 1.00 | 155.85 |
| 2771 | CA | VAL A | 155 | 0.813 | 47.372 | −15.153 | 1.00 | 155.85 |
| 2772 | CB | VAL A | 155 | 0.510 | 45.884 | −15.201 | 1.00 | 196.06 |
| 2773 | CG1 | VAL A | 155 | 1.673 | 45.144 | −15.841 | 1.00 | 196.06 |
| 2774 | CG2 | VAL A | 155 | 0.273 | 45.373 | −13.809 | 1.00 | 196.06 |
| 2775 | C | VAL A | 155 | 0.960 | 47.894 | −16.560 | 1.00 | 155.85 |
| 2776 | O | VAL A | 155 | −0.013 | 47.951 | −17.309 | 1.00 | 155.85 |
| 2777 | N | TRP A | 156 | 2.187 | 48.251 | −16.912 | 1.00 | 136.77 |
| 2778 | CA | TRP A | 156 | 2.437 | 48.821 | −18.215 | 1.00 | 136.77 |
| 2779 | CB | TRP A | 156 | 1.888 | 47.941 | −19.308 | 1.00 | 169.17 |
| 2780 | CG | TRP A | 156 | 2.584 | 46.701 | −19.394 | 1.00 | 169.17 |
| 2781 | CD2 | TRP A | 156 | 3.991 | 46.538 | −19.596 | 1.00 | 169.17 |
| 2782 | CE2 | TRP A | 156 | 4.260 | 45.184 | −19.532 | 1.00 | 169.17 |
| 2783 | CE3 | TRP A | 156 | 5.037 | 47.423 | −19.855 | 1.00 | 169.17 |
| 2784 | CD1 | TRP A | 156 | 2.066 | 45.478 | −19.202 | 1.00 | 169.17 |
| 2785 | NE1 | TRP A | 156 | 3.053 | 44.565 | −19.283 | 1.00 | 169.17 |
| 2786 | CZ2 | TRP A | 156 | 5.536 | 44.634 | −19.734 | 1.00 | 169.17 |
| 2787 | CZ3 | TRP A | 156 | 6.293 | 46.924 | −20.012 | 1.00 | 169.17 |
| 2788 | CH2 | TRP A | 156 | 6.542 | 45.522 | −19.971 | 1.00 | 169.17 |
| 2789 | C | TRP A | 156 | 1.664 | 50.102 | −18.251 | 1.00 | 136.77 |
| 2790 | O | TRP A | 156 | 2.130 | 51.132 | −17.775 | 1.00 | 136.77 |
| 2791 | N | GLN A | 157 | 0.445 | 50.004 | −18.777 | 1.00 | 192.06 |
| 2792 | CA | GLN A | 157 | −0.395 | 51.166 | −18.902 | 1.00 | 192.06 |
| 2793 | CB | GLN A | 157 | −0.133 | 51.791 | −20.263 | 1.00 | 249.57 |
| 2794 | CG | GLN A | 157 | 1.231 | 52.449 | −20.291 | 1.00 | 249.57 |
| 2795 | CD | GLN A | 157 | 1.374 | 53.420 | −19.126 | 1.00 | 249.57 |
| 2796 | OE1 | GLN A | 157 | 0.539 | 54.293 | −18.950 | 1.00 | 249.57 |
| 2797 | NE2 | GLN A | 157 | 2.428 | 53.271 | −18.339 | 1.00 | 249.57 |
| 2798 | C | GLN A | 157 | −1.873 | 50.913 | −18.673 | 1.00 | 192.06 |
| 2799 | O | GLN A | 157 | −2.717 | 51.753 | −18.989 | 1.00 | 192.06 |
| 2800 | N | LEU A | 158 | −2.181 | 49.753 | −18.108 | 1.00 | 96.98 |
| 2801 | CA | LEU A | 158 | −3.565 | 49.394 | −17.813 | 1.00 | 96.98 |
| 2802 | CB | LEU A | 158 | −4.018 | 48.234 | −18.697 | 1.00 | 92.50 |
| 2803 | CG | LEU A | 158 | −4.362 | 48.530 | −20.148 | 1.00 | 92.50 |
| 2804 | CD1 | LEU A | 158 | −5.577 | 47.691 | −20.496 | 1.00 | 92.50 |
| 2805 | CD2 | LEU A | 158 | −4.695 | 50.015 | −20.343 | 1.00 | 92.50 |
| 2806 | C | LEU A | 158 | −3.758 | 49.028 | −16.345 | 1.00 | 96.98 |
| 2807 | O | LEU A | 158 | −2.821 | 48.596 | −15.661 | 1.00 | 96.98 |
| 2808 | N | ASP A | 159 | −4.983 | 49.216 | −15.869 | 1.00 | 119.40 |
| 2809 | CA | ASP A | 159 | −5.330 | 48.925 | −14.485 | 1.00 | 119.40 |
| 2810 | CB | ASP A | 159 | −6.442 | 49.875 | −14.018 | 1.00 | 201.99 |
| 2811 | CG | ASP A | 159 | −6.096 | 51.345 | −14.236 | 1.00 | 201.99 |
| 2812 | OD1 | ASP A | 159 | −5.103 | 51.832 | −13.651 | 1.00 | 201.99 |
| 2813 | OD2 | ASP A | 159 | −6.823 | 52.018 | −15.000 | 1.00 | 201.99 |
| 2814 | C | ASP A | 159 | −5.793 | 47.473 | −14.334 | 1.00 | 119.40 |
| 2815 | O | ASP A | 159 | −6.417 | 46.919 | −15.235 | 1.00 | 119.40 |
| 2816 | N | TYR A | 160 | −5.475 | 46.860 | −13.197 | 1.00 | 117.93 |
| 2817 | CA | TYR A | 160 | −5.875 | 45.481 | −12.929 | 1.00 | 117.93 |
| 2818 | CB | TYR A | 160 | −4.769 | 44.503 | −13.308 | 1.00 | 127.75 |
| 2819 | CG | TYR A | 160 | −4.261 | 44.648 | −14.715 | 1.00 | 127.75 |
| 2820 | CD1 | TYR A | 160 | −3.184 | 45.483 | −14.997 | 1.00 | 127.75 |
| 2821 | CE1 | TYR A | 160 | −2.707 | 45.625 | −16.277 | 1.00 | 127.75 |
| 2822 | CD2 | TYR A | 160 | −4.855 | 43.952 | −15.766 | 1.00 | 127.75 |
| 2823 | CE2 | TYR A | 160 | −4.386 | 44.087 | −17.061 | 1.00 | 127.75 |
| 2824 | CZ | TYR A | 160 | −3.309 | 44.932 | −17.297 | 1.00 | 127.75 |
| 2825 | OH | TYR A | 160 | −2.808 | 45.145 | −18.541 | 1.00 | 127.75 |
| 2826 | C | TYR A | 160 | −6.239 | 45.240 | −11.471 | 1.00 | 117.93 |
| 2827 | O | TYR A | 160 | −5.674 | 45.836 | −10.557 | 1.00 | 117.93 |
| 2828 | N | GLU A | 161 | −7.184 | 44.335 | −11.275 | 1.00 | 99.07 |
| 2829 | CA | GLU A | 161 | −7.665 | 43.963 | −9.960 | 1.00 | 99.07 |
| 2830 | CB | GLU A | 161 | −9.179 | 44.113 | −9.945 | 1.00 | 160.66 |
| 2831 | CG | GLU A | 161 | −9.877 | 43.683 | −8.681 | 1.00 | 160.66 |
| 2832 | CD | GLU A | 161 | −11.290 | 44.226 | −8.624 | 1.00 | 160.66 |
| 2833 | OE1 | GLU A | 161 | −12.117 | 43.690 | −7.856 | 1.00 | 160.66 |
| 2834 | OE2 | GLU A | 161 | −11.564 | 45.206 | −9.350 | 1.00 | 160.66 |
| 2835 | C | GLU A | 161 | −7.258 | 42.507 | −9.699 | 1.00 | 99.07 |
| 2836 | O | GLU A | 161 | −7.346 | 41.672 | −10.598 | 1.00 | 99.07 |
| 2837 | N | SER A | 162 | −6.806 | 42.206 | −8.481 | 1.00 | 84.68 |
| 2838 | CA | SER A | 162 | −6.378 | 40.856 | −8.119 | 1.00 | 84.68 |
| 2839 | CB | SER A | 162 | −5.247 | 40.923 | −7.102 | 1.00 | 134.29 |
| 2840 | OG | SER A | 162 | −5.670 | 41.599 | −5.932 | 1.00 | 134.29 |
| 2841 | C | SER A | 162 | −7.520 | 40.029 | −7.536 | 1.00 | 84.68 |
| 2842 | O | SER A | 162 | −8.592 | 40.555 | −7.230 | 1.00 | 84.68 |
| 2843 | N | GLU A | 163 | −7.292 | 38.729 | −7.382 | 1.00 | 56.84 |
| 2844 | CA | GLU A | 163 | −8.316 | 37.829 | −6.842 | 1.00 | 56.84 |
| 2845 | CB | GLU A | 163 | −7.885 | 36.370 | −7.015 | 1.00 | 162.97 |
| 2846 | CG | GLU A | 163 | −7.984 | 35.836 | −8.438 | 1.00 | 162.97 |
| 2847 | CD | GLU A | 163 | −9.417 | 35.601 | −8.869 | 1.00 | 162.97 |
| 2848 | OE1 | GLU A | 163 | −10.122 | 34.835 | −8.175 | 1.00 | 162.97 |
| 2849 | OE2 | GLU A | 163 | −9.835 | 36.176 | −9.900 | 1.00 | 162.97 |
| 2850 | C | GLU A | 163 | −8.437 | 38.151 | −5.368 | 1.00 | 56.84 |
| 2851 | O | GLU A | 163 | −7.439 | 38.433 | −4.720 | 1.00 | 56.84 |
| 2852 | N | PRO A | 164 | −9.660 | 38.122 | −4.805 | 1.00 | 48.99 |
| 2853 | CD | PRO A | 164 | −10.959 | 37.760 | −5.379 | 1.00 | 93.04 |
| 2854 | CA | PRO A | 164 | −9.789 | 38.423 | −3.371 | 1.00 | 48.99 |
| 2855 | CB | PRO A | 164 | −11.295 | 38.547 | −3.196 | 1.00 | 93.04 |
| 2856 | CG | PRO A | 164 | −11.814 | 37.544 | −4.135 | 1.00 | 93.04 |
| 2857 | C | PRO A | 164 | −9.201 | 37.307 | −2.528 | 1.00 | 48.99 |
| 2858 | O | PRO A | 164 | −9.101 | 36.160 | −2.977 | 1.00 | 48.99 |
| 2859 | N | LEU A | 165 | −8.802 | 37.625 | −1.303 | 1.00 | 69.69 |
| 2860 | CA | LEU A | 165 | −8.236 | 36.609 | −0.426 | 1.00 | 69.69 |
| 2861 | CB | LEU A | 165 | −6.733 | 36.661 | −0.465 | 1.00 | 37.26 |
| 2862 | CG | LEU A | 165 | −6.041 | 35.774 | 0.560 | 1.00 | 37.26 |
| 2863 | CD1 | LEU A | 165 | −6.655 | 34.407 | 0.459 | 1.00 | 37.26 |
| 2864 | CD2 | LEU A | 165 | −4.535 | 35.665 | 0.301 | 1.00 | 37.26 |
| 2865 | C | LEU A | 165 | −8.661 | 36.800 | 1.000 | 1.00 | 69.69 |
| 2866 | O | LEU A | 165 | −8.430 | 37.863 | 1.562 | 1.00 | 69.69 |
| 2867 | N | ASN A | 166 | −9.272 | 35.777 | 1.585 | 1.00 | 67.70 |
| 2868 | CA | ASN A | 166 | −9.725 | 35.861 | 2.962 | 1.00 | 67.70 |
| 2869 | CB | ASN A | 166 | −10.806 | 34.849 | 3.224 | 1.00 | 76.12 |
| 2870 | CG | ASN A | 166 | −12.182 | 35.396 | 2.998 | 1.00 | 76.12 |
| 2871 | OD1 | ASN A | 166 | −12.414 | 36.571 | 3.171 | 1.00 | 76.12 |
| 2872 | ND2 | ASN A | 166 | −13.106 | 34.519 | 2.633 | 1.00 | 76.12 |
| 2873 | C | ASN A | 166 | −8.606 | 35.605 | 3.943 | 1.00 | 67.70 |
| 2874 | O | ASN A | 166 | −7.724 | 34.792 | 3.687 | 1.00 | 67.70 |
| 2875 | N | ILE A | 167 | −8.665 | 36.273 | 5.090 | 1.00 | 54.98 |
| 2876 | CA | ILE A | 167 | −7.634 | 36.127 | 6.105 | 1.00 | 54.98 |
| 2877 | CB | ILE A | 167 | −6.686 | 37.292 | 6.049 | 1.00 | 41.48 |
| 2878 | CG2 | ILE A | 167 | −6.883 | 37.357 | 7.277 | 1.00 | 41.48 |
| 2879 | CG1 | ILE A | 167 | −5.770 | 37.131 | 4.855 | 1.00 | 41.48 |
| 2880 | CD1 | ILE A | 167 | −4.655 | 38.164 | 4.815 | 1.00 | 41.48 |
| 2881 | C | ILE A | 167 | −8.248 | 36.093 | 7.478 | 1.00 | 54.98 |
| 2882 | O | ILE A | 167 | −9.113 | 36.914 | 7.783 | 1.00 | 54.98 |
| 2883 | N | THR A | 168 | −7.820 | 35.163 | 8.317 | 1.00 | 55.70 |
| 2884 | CA | THR A | 168 | −8.391 | 35.122 | 9.642 | 1.00 | 55.70 |
| 2885 | CB | THR A | 168 | −9.241 | 33.875 | 9.837 | 1.00 | 71.33 |
| 2886 | OG1 | THR A | 168 | −10.289 | 33.866 | 8.860 | 1.00 | 71.33 |
| 2887 | CG2 | THR A | 168 | −9.857 | 33.869 | 11.209 | 1.00 | 71.33 |
| 2888 | C | THR A | 168 | −7.339 | 35.171 | 10.697 | 1.00 | 55.70 |
| 2889 | O | THR A | 168 | −6.295 | 34.539 | 10.587 | 1.00 | 55.70 |
| 2890 | N | VAL A | 169 | −7.618 | 35.950 | 11.724 | 1.00 | 64.35 |
| 2891 | CA | VAL A | 169 | −6.725 | 36.090 | 12.863 | 1.00 | 64.35 |
| 2892 | CB | VAL A | 169 | −6.370 | 37.560 | 13.087 | 1.00 | 77.36 |
| 2893 | CG1 | VAL A | 169 | −5.895 | 37.772 | 14.468 | 1.00 | 77.36 |
| 2894 | CG2 | VAL A | 169 | −5.314 | 37.978 | 12.128 | 1.00 | 77.36 |
| 2895 | C | VAL A | 169 | −7.539 | 35.567 | 14.048 | 1.00 | 64.35 |
| 2896 | O | VAL A | 169 | −8.510 | 36.203 | 14.463 | 1.00 | 64.35 |
| 2897 | N | ILE A | 170 | −7.175 | 34.395 | 14.562 | 1.00 | 76.83 |
| 2898 | CA | ILE A | 170 | −7.889 | 33.797 | 15.690 | 1.00 | 76.83 |
| 2899 | CB | ILE A | 170 | −7.898 | 32.250 | 15.590 | 1.00 | 133.66 |
| 2900 | CG2 | ILE A | 170 | −8.437 | 31.821 | 14.237 | 1.00 | 133.66 |
| 2901 | CG1 | ILE A | 170 | −6.482 | 31.697 | 15.761 | 1.00 | 133.66 |
| 2902 | CD1 | ILE A | 170 | −6.386 | 30.180 | 15.647 | 1.00 | 133.66 |
| 2903 | C | ILE A | 170 | −7.196 | 34.228 | 16.976 | 1.00 | 76.83 |
| 2904 | O | ILE A | 170 | −6.164 | 34.887 | 16.922 | 1.00 | 76.83 |
| 2905 | N | LYS A | 171 | −7.757 | 33.870 | 18.127 | 1.00 | 125.94 |
| 2906 | CA | LYS A | 171 | −7.152 | 34.252 | 19.397 | 1.00 | 125.94 |
| 2907 | CB | LYS A | 171 | −8.004 | 35.328 | 20.069 | 1.00 | 198.00 |
| 2908 | CG | LYS A | 171 | −9.449 | 34.922 | 20.293 | 1.00 | 198.00 |
| 2909 | CD | LYS A | 171 | −10.354 | 36.141 | 20.399 | 1.00 | 198.00 |
| 2910 | CE | LYS A | 171 | −9.952 | 37.059 | 21.546 | 1.00 | 198.00 |
| 2911 | NZ | LYS A | 171 | −10.825 | 38.268 | 21.607 | 1.00 | 198.00 |
| 2912 | C | LYS A | 171 | −6.957 | 33.072 | 20.338 | 1.00 | 125.94 |
| 2913 | O | LYS A | 171 | −6.326 | 33.204 | 21.388 | 1.00 | 125.94 |
| 2914 | C1 | NAG A | 221 | 13.561 | 29.146 | −11.328 | 1.00 | 244.51 |
| 2915 | C2 | NAG A | 221 | 13.758 | 30.631 | −11.596 | 1.00 | 244.51 |
| 2916 | N2 | NAG A | 221 | 12.475 | 31.303 | −11.575 | 1.00 | 244.51 |
| 2917 | C7 | NAG A | 221 | 12.407 | 32.594 | −11.273 | 1.00 | 244.51 |
| 2918 | O7 | NAG A | 221 | 13.396 | 33.270 | −10.988 | 1.00 | 244.51 |
| 2919 | C8 | NAG A | 221 | 11.029 | 33.233 | −11.281 | 1.00 | 244.51 |
| 2920 | C3 | NAG A | 221 | 14.405 | 30.847 | −12.952 | 1.00 | 244.51 |
| 2921 | O3 | NAG A | 221 | 14.740 | 32.219 | −13.099 | 1.00 | 244.51 |
| 2922 | C4 | NAG A | 221 | 15.661 | 29.997 | −13.135 | 1.00 | 244.51 |
| 2923 | O4 | NAG A | 221 | 16.044 | 30.091 | −14.520 | 1.00 | 244.51 |
| 2924 | C5 | NAG A | 221 | 15.375 | 28.530 | −12.759 | 1.00 | 244.51 |
| 2925 | O5 | NAG A | 221 | 14.809 | 28.456 | −11.436 | 1.00 | 244.51 |
| 2926 | C6 | NAG A | 221 | 16.622 | 27.665 | −12.740 | 1.00 | 244.51 |
| 2927 | O6 | NAG A | 221 | 17.566 | 28.140 | −11.790 | 1.00 | 244.51 |
| 2928 | C1 | NAG A | 222 | 17.330 | 29.723 | −14.890 | 1.00 | 195.02 |
| 2929 | C2 | NAG A | 222 | 17.910 | 30.770 | −15.864 | 1.00 | 195.02 |
| 2930 | N2 | NAG A | 222 | 17.966 | 32.078 | −15.229 | 1.00 | 195.02 |
| 2931 | C7 | NAG A | 222 | 19.134 | 32.692 | −15.052 | 1.00 | 195.02 |
| 2932 | O7 | NAG A | 222 | 20.210 | 32.206 | −15.404 | 1.00 | 195.02 |
| 2933 | C8 | NAG A | 222 | 19.106 | 34.055 | −14.383 | 1.00 | 195.02 |
| 2934 | C3 | NAG A | 222 | 17.061 | 30.835 | −17.148 | 1.00 | 195.02 |
| 2935 | O3 | NAG A | 222 | 17.694 | 31.675 | −18.105 | 1.00 | 195.02 |
| 2936 | C4 | NAG A | 222 | 16.869 | 29.431 | −17.744 | 1.00 | 195.02 |
| 2937 | O4 | NAG A | 222 | 15.938 | 29.494 | −18.814 | 1.00 | 195.02 |
| 2938 | C5 | NAG A | 222 | 16.356 | 28.454 | −16.676 | 1.00 | 195.02 |
| 2939 | O5 | NAG A | 222 | 17.249 | 28.441 | −15.538 | 1.00 | 195.02 |
| 2940 | C6 | NAG A | 222 | 16.248 | 27.029 | −17.174 | 1.00 | 195.02 |
| 2941 | O6 | NAG A | 222 | 15.013 | 26.448 | −16.789 | 1.00 | 195.02 |
| 2942 | C1 | NAG A | 242 | −3.473 | 17.670 | −6.472 | 1.00 | 81.55 |
| 2943 | C2 | NAG A | 242 | −3.080 | 17.582 | −7.921 | 1.00 | 81.55 |
| 2944 | N2 | NAG A | 242 | −1.712 | 17.148 | −8.025 | 1.00 | 81.55 |
| 2945 | C7 | NAG A | 242 | −1.420 | 16.075 | −8.748 | 1.00 | 81.55 |
| 2946 | O7 | NAG A | 242 | −2.270 | 15.414 | −9.324 | 1.00 | 81.55 |
| 2947 | C8 | NAG A | 242 | 0.033 | 15.657 | −8.846 | 1.00 | 81.55 |
| 2948 | C3 | NAG A | 242 | −3.225 | 18.933 | −8.583 | 1.00 | 81.55 |
| 2949 | O3 | NAG A | 242 | −2.918 | 18.814 | −9.969 | 1.00 | 81.55 |
| 2950 | C4 | NAG A | 242 | −4.642 | 19.456 | −8.403 | 1.00 | 81.55 |
| 2951 | O4 | NAG A | 242 | −4.712 | 20.825 | −8.846 | 1.00 | 81.55 |
| 2952 | C5 | NAG A | 242 | −5.062 | 19.392 | −6.945 | 1.00 | 81.55 |
| 2953 | O5 | NAG A | 242 | −4.830 | 18.086 | −6.394 | 1.00 | 81.55 |
| 2954 | C6 | NAG A | 242 | −6.547 | 19.630 | −6.824 | 1.00 | 81.55 |
| 2955 | O6 | NAG A | 242 | −6.826 | 20.697 | −5.933 | 1.00 | 81.55 |
| 2956 | C1 | NAG A | 243 | −5.536 | 21.071 | −9.934 | 1.00 | 123.88 |
| 2957 | C2 | NAG A | 243 | −6.020 | 22.528 | −9.929 | 1.00 | 123.88 |
| 2958 | N2 | NAG A | 243 | −6.814 | 22.800 | −8.743 | 1.00 | 123.88 |
| 2959 | C7 | NAG A | 243 | −6.607 | 23.908 | −8.041 | 1.00 | 123.88 |
| 2960 | O7 | NAG A | 243 | −5.746 | 24.727 | −8.337 | 1.00 | 123.88 |
| 2961 | C8 | NAG A | 243 | −7.482 | 24.135 | −6.820 | 1.00 | 123.88 |
| 2962 | C3 | NAG A | 243 | −6.875 | 22.766 | −11.173 | 1.00 | 123.88 |
| 2963 | O3 | NAG A | 243 | −7.276 | 24.126 | −11.231 | 1.00 | 123.88 |
| 2964 | C4 | NAG A | 243 | −6.109 | 22.379 | −12.449 | 1.00 | 123.88 |
| 2965 | O4 | NAG A | 243 | −7.002 | 22.470 | −13.597 | 1.00 | 123.88 |
| 2966 | C5 | NAG A | 243 | −5.608 | 20.937 | −12.312 | 1.00 | 123.88 |
| 2967 | O5 | NAG A | 243 | −4.793 | 20.809 | −11.132 | 1.00 | 123.88 |
| 2968 | C6 | NAG A | 243 | −4.789 | 20.444 | −13.485 | 1.00 | 123.88 |
| 2969 | O6 | NAG A | 243 | −3.560 | 21.141 | −13.577 | 1.00 | 123.88 |
| 2970 | C1 | MAN A | 244 | −6.640 | 23.134 | −14.739 | 1.00 | 177.21 |
| 2971 | C2 | MAN A | 244 | −6.289 | 24.639 | −14.645 | 1.00 | 177.21 |
| 2972 | O2 | MAN A | 244 | −4.892 | 24.794 | −14.586 | 1.00 | 177.21 |
| 2973 | C3 | MAN A | 244 | −6.845 | 25.182 | −15.998 | 1.00 | 177.21 |
| 2974 | O3 | MAN A | 244 | −6.636 | 26.575 | −16.149 | 1.00 | 177.21 |
| 2975 | C4 | MAN A | 244 | −6.314 | 24.396 | −17.244 | 1.00 | 177.21 |
| 2976 | O4 | MAN A | 244 | −6.840 | 24.954 | −18.451 | 1.00 | 177.21 |
| 2977 | C5 | MAN A | 244 | −6.779 | 22.928 | −17.096 | 1.00 | 177.21 |
| 2978 | O5 | MAN A | 244 | −6.232 | 22.337 | −15.891 | 1.00 | 177.21 |
| 2979 | C6 | MAN A | 244 | −6.487 | 22.037 | −18.309 | 1.00 | 177.21 |
| 2980 | O6 | MAN A | 244 | −5.159 | 21.562 | −18.301 | 1.00 | 177.21 |
| 2981 | C1 | NAG A | 250 | 18.849 | 18.682 | −1.016 | 1.00 | 245.89 |
| 2982 | C2 | NAG A | 250 | 19.989 | 19.613 | −0.566 | 1.00 | 245.89 |
| 2983 | N2 | NAG A | 250 | 20.115 | 19.601 | 0.880 | 1.00 | 245.89 |
| 2984 | C7 | NAG A | 250 | 21.178 | 19.048 | 1.458 | 1.00 | 245.89 |
| 2985 | O7 | NAG A | 250 | 22.091 | 18.518 | 0.819 | 1.00 | 245.89 |
| 2986 | C8 | NAG A | 250 | 21.237 | 19.081 | 2.980 | 1.00 | 245.89 |
| 2987 | C3 | NAG A | 250 | 19.696 | 21.039 | −1.050 | 1.00 | 245.89 |
| 2988 | O3 | NAG A | 250 | 20.782 | 21.896 | −0.728 | 1.00 | 245.89 |
| 2989 | O4 | NAG A | 250 | 19.457 | 21.047 | −2.564 | 1.00 | 245.89 |
| 2990 | O4 | NAG A | 250 | 19.058 | 22.347 | −2.977 | 1.00 | 245.89 |
| 2991 | C5 | NAG A | 250 | 18.367 | 20.028 | −2.935 | 1.00 | 245.89 |
| 2992 | O5 | NAG A | 250 | 18.721 | 18.715 | −2.444 | 1.00 | 245.89 |
| 2993 | C6 | NAG A | 250 | 18.165 | 19.903 | −4.436 | 1.00 | 245.89 |
| 2994 | O6 | NAG A | 250 | 17.400 | 18.748 | −4.760 | 1.00 | 245.89 |
| 2995 | C1 | NAG A | 274 | 2.176 | 9.666 | 16.692 | 1.00 | 235.37 |
| 2996 | C2 | NAG A | 274 | 1.514 | 10.512 | 17.789 | 1.00 | 235.37 |
| 2997 | N2 | NAG A | 274 | 2.519 | 11.269 | 18.514 | 1.00 | 235.37 |
| 2998 | C7 | NAG A | 274 | 2.186 | 12.397 | 19.137 | 1.00 | 235.37 |
| 2999 | O7 | NAG A | 274 | 1.042 | 12.855 | 19.128 | 1.00 | 235.37 |
| 3000 | C8 | NAG A | 274 | 3.289 | 13.134 | 19.882 | 1.00 | 235.37 |
| 3001 | C3 | NAG A | 274 | 0.750 | 9.604 | 18.761 | 1.00 | 235.37 |
| 3002 | O3 | NAG A | 274 | 0.023 | 10.398 | 19.687 | 1.00 | 235.37 |
| 3003 | C4 | NAG A | 274 | −0.216 | 8.687 | 18.005 | 1.00 | 235.37 |
| 3004 | O4 | NAG A | 274 | −0.794 | 7.758 | 18.909 | 1.00 | 235.37 |
| 3005 | C5 | NAG A | 274 | 0.534 | 7.934 | 16.900 | 1.00 | 235.37 |
| 3006 | O5 | NAG A | 274 | 1.187 | 8.871 | 16.018 | 1.00 | 235.37 |
| 3007 | C6 | NAG A | 274 | −0.384 | 7.085 | 16.044 | 1.00 | 235.37 |
| 3008 | O6 | NAG A | 274 | 0.294 | 6.598 | 14.895 | 1.00 | 235.37 |
| 3009 | C1 | NAG A | 335 | 7.685 | 42.617 | −1.591 | 1.00 | 248.30 |
| 3010 | C2 | NAG A | 335 | 8.870 | 42.060 | −0.765 | 1.00 | 248.30 |
| 3011 | N2 | NAG A | 335 | 8.767 | 42.587 | 0.583 | 1.00 | 248.30 |
| 3012 | C7 | NAG A | 335 | 8.573 | 41.777 | 1.618 | 1.00 | 248.30 |
| 3013 | O7 | NAG A | 335 | 8.483 | 40.553 | 11.511 | 1.00 | 248.30 |
| 3014 | C8 | NAG A | 335 | 8.472 | 42.430 | 2.987 | 1.00 | 248.30 |
| 3015 | C3 | NAG A | 335 | 10.258 | 42.417 | −1.325 | 1.00 | 248.30 |
| 3016 | O3 | NAG A | 335 | 11.229 | 41.541 | −0.771 | 1.00 | 248.30 |
| 3017 | C4 | NAG A | 335 | 10.290 | 42.300 | −2.841 | 1.00 | 248.30 |
| 3018 | O4 | NAG A | 335 | 11.560 | 42.706 | −3.329 | 1.00 | 248.30 |
| 3019 | C5 | NAG A | 335 | 9.195 | 43.189 | −3.414 | 1.00 | 248.30 |
| 3020 | O5 | NAG A | 335 | 7.904 | 42.673 | −3.021 | 1.00 | 248.30 |
| 3021 | C6 | NAG A | 335 | 9.222 | 43.210 | −4.935 | 1.00 | 248.30 |
| 3022 | O6 | NAG A | 335 | 9.423 | 44.524 | −5.434 | 1.00 | 248.30 |
| 3023 | C1 | NAG A | 340 | 0.521 | 43.731 | 20.574 | 1.00 | 249.48 |
| 3024 | C2 | NAG A | 340 | −0.261 | 42.929 | 21.588 | 1.00 | 249.48 |
| 3025 | N2 | NAG A | 340 | −1.284 | 42.144 | 20.930 | 1.00 | 249.48 |
| 3026 | C7 | NAG A | 340 | −1.377 | 40.843 | 21.191 | 1.00 | 249.48 |
| 3027 | O7 | NAG A | 340 | −0.627 | 40.266 | 21.988 | 1.00 | 249.48 |
| 3028 | C8 | NAG A | 340 | −2.460 | 40.060 | 20.472 | 1.00 | 249.48 |
| 3029 | C3 | NAG A | 340 | −0.877 | 43.866 | 22.605 | 1.00 | 249.48 |
| 3030 | O3 | NAG A | 340 | −1.567 | 43.103 | 23.596 | 1.00 | 249.48 |
| 3031 | C4 | NAG A | 340 | 0.234 | 44.689 | 23.266 | 1.00 | 249.48 |
| 3032 | O4 | NAG A | 340 | −0.370 | 45.703 | 24.068 | 1.00 | 249.48 |
| 3033 | C5 | NAG A | 340 | 1.188 | 45.334 | 22.220 | 1.00 | 249.48 |
| 3034 | O5 | NAG A | 340 | 1.601 | 44.382 | 21.233 | 1.00 | 249.48 |
| 3035 | C6 | NAG A | 340 | 2.460 | 45.780 | 22.881 | 1.00 | 249.48 |
| 3036 | O6 | NAG A | 340 | 3.548 | 45.816 | 21.985 | 1.00 | 249.48 |
| 3037 | C1 | NAG A | 366 | −14.447 | 34.952 | 2.337 | 1.00 | 170.79 |
| 3038 | C2 | NAG A | 366 | −15.009 | 34.055 | 1.250 | 1.00 | 170.79 |
| 3039 | N2 | NAG A | 366 | −14.171 | 34.149 | 0.073 | 1.00 | 170.79 |
| 3040 | C7 | NAG A | 366 | −13.171 | 33.289 | −0.105 | 1.00 | 170.79 |
| 3041 | O7 | NAG A | 366 | −12.912 | 32.383 | 0.691 | 1.00 | 170.79 |
| 3042 | C8 | NAG A | 366 | −12.329 | 33.454 | −1.361 | 1.00 | 170.79 |
| 3043 | C3 | NAG A | 366 | −16.425 | 34.482 | 0.910 | 1.00 | 170.79 |
| 3044 | O3 | NAG A | 366 | −16.997 | 33.542 | 0.014 | 1.00 | 170.79 |
| 3045 | C4 | NAG A | 366 | −17.290 | 34.565 | 2.168 | 1.00 | 170.79 |
| 3046 | O4 | NAG A | 366 | −18.549 | 35.187 | 1.824 | 1.00 | 170.79 |
| 3047 | C5 | NAG A | 366 | −16.584 | 35.380 | 3.275 | 1.00 | 170.79 |
| 3048 | O5 | NAG A | 366 | −15.258 | 34.873 | 3.503 | 1.00 | 170.79 |
| 3049 | C6 | NAG A | 366 | −17.297 | 35.315 | 4.613 | 1.00 | 170.79 |
| 3050 | O6 | NAG A | 366 | −16.620 | 36.092 | 5.592 | 1.00 | 170.79 |
| 3051 | C1 | NAG A | 367 | −19.711 | 34.493 | 2.163 | 1.00 | 247.02 |
| 3052 | C2 | NAG A | 367 | −20.892 | 35.462 | 2.268 | 1.00 | 247.02 |
| 3053 | N2 | NAG A | 367 | −20.619 | 36.488 | 3.255 | 1.00 | 247.02 |
| 3054 | C7 | NAG A | 367 | −20.363 | 37.730 | 2.856 | 1.00 | 247.02 |
| 3055 | O7 | NAG A | 367 | −20.347 | 38.061 | 1.668 | 1.00 | 247.02 |
| 3056 | C8 | NAG A | 367 | −20.084 | 38.762 | 3.937 | 1.00 | 247.02 |
| 3057 | C3 | NAG A | 367 | −22.151 | 34.676 | 2.640 | 1.00 | 247.02 |
| 3058 | O3 | NAG A | 367 | −23.265 | 35.554 | 2.696 | 1.00 | 247.02 |
| 3059 | C4 | NAG A | 367 | −22.395 | 33.586 | 1.591 | 1.00 | 247.02 |
| 3060 | O4 | NAG A | 367 | −23.511 | 32.793 | 1.970 | 1.00 | 247.02 |
| 3061 | C5 | NAG A | 367 | −21.148 | 32.698 | 1.448 | 1.00 | 247.02 |
| 3062 | O5 | NAG A | 367 | −19.981 | 33.508 | 1.147 | 1.00 | 247.02 |
| 3063 | C6 | NAG A | 367 | −21.291 | 31.682 | 0.332 | 1.00 | 247.02 |
| 3064 | O6 | NAG A | 367 | −20.416 | 31.974 | −0.749 | 1.00 | 247.02 |
| 3065 | CB | LYS B | 4 | 28.538 | 57.342 | 22.861 | 1.00 | 248.35 |
| 3066 | CG | LYS B | 4 | 28.723 | 58.799 | 22.474 | 1.00 | 248.35 |
| 3067 | CD | LYS B | 4 | 28.723 | 59.692 | 23.702 | 1.00 | 248.35 |
| 3068 | CE | LYS B | 4 | 28.914 | 61.151 | 23.330 | 1.00 | 248.35 |
| 3069 | NZ | LYS B | 4 | 28.914 | 62.022 | 24.537 | 1.00 | 248.35 |
| 3070 | C | LYS B | 4 | 29.934 | 56.599 | 20.941 | 1.00 | 249.33 |
| 3071 | O | LYS B | 4 | 30.913 | 57.081 | 21.514 | 1.00 | 249.33 |
| 3072 | N | LYS B | 4 | 28.491 | 54.970 | 22.165 | 1.00 | 249.33 |
| 3073 | CA | LYS B | 4 | 28.619 | 56.377 | 21.683 | 1.00 | 249.33 |
| 3074 | N | PRO B | 5 | 29.974 | 56.244 | 19.648 | 1.00 | 115.49 |
| 3075 | CD | PRO B | 5 | 28.994 | 55.392 | 18.958 | 1.00 | 70.51 |
| 3076 | CA | PRO B | 5 | 31.186 | 56.414 | 18.835 | 1.00 | 115.49 |
| 3077 | CB | PRO B | 5 | 31.037 | 55.337 | 17.765 | 1.00 | 70.51 |
| 3078 | CG | PRO B | 5 | 29.573 | 55.299 | 17.553 | 1.00 | 70.51 |
| 3079 | C | PRO B | 5 | 31.329 | 57.807 | 18.247 | 1.00 | 115.49 |
| 3080 | O | PRO B | 5 | 30.350 | 58.537 | 18.126 | 1.00 | 115.49 |
| 3081 | N | LYS B | 6 | 32.553 | 58.174 | 17.885 | 1.00 | 105.72 |
| 3082 | CA | LYS B | 6 | 32.811 | 59.492 | 17.331 | 1.00 | 105.72 |
| 3083 | CB | LYS B | 6 | 33.258 | 60.458 | 18.449 | 1.00 | 206.94 |
| 3084 | CG | LYS B | 6 | 33.432 | 61.901 | 17.991 | 1.00 | 206.94 |
| 3085 | CD | LYS B | 6 | 33.697 | 62.850 | 19.152 | 1.00 | 206.94 |
| 3086 | CE | LYS B | 6 | 33.826 | 64.287 | 18.654 | 1.00 | 206.94 |
| 3087 | NZ | LYS B | 6 | 34.006 | 65.275 | 19.759 | 1.00 | 206.94 |
| 3088 | C | LYS B | 6 | 33.857 | 59.429 | 16.222 | 1.00 | 105.72 |
| 3089 | O | LYS B | 6 | 35.018 | 59.109 | 16.467 | 1.00 | 105.72 |
| 3090 | N | VAL B | 7 | 33.433 | 59.742 | 15.002 | 1.00 | 68.17 |
| 3091 | CA | VAL B | 7 | 34.317 | 59.728 | 13.843 | 1.00 | 68.17 |
| 3092 | CB | VAL B | 7 | 33.553 | 59.960 | 12.545 | 1.00 | 86.47 |
| 3093 | CG1 | VAL B | 7 | 34.487 | 59.738 | 11.359 | 1.00 | 86.47 |
| 3094 | CG2 | VAL B | 7 | 32.346 | 59.072 | 12.479 | 1.00 | 86.47 |
| 3095 | C | VAL B | 7 | 35.401 | 60.796 | 13.845 | 1.00 | 68.17 |
| 3096 | O | VAL B | 7 | 35.094 | 61.988 | 13.803 | 1.00 | 68.17 |
| 3097 | N | SER B | 8 | 36.661 | 60.385 | 13.864 | 1.00 | 61.68 |
| 3098 | CA | SER B | 8 | 37.741 | 61.355 | 13.837 | 1.00 | 61.68 |
| 3099 | CB | SER B | 8 | 38.836 | 60.969 | 14.842 | 1.00 | 135.50 |
| 3100 | OG | SER B | 8 | 39.289 | 59.647 | 14.622 | 1.00 | 135.50 |
| 3101 | C | SER B | 8 | 38.303 | 61.405 | 12.406 | 1.00 | 61.68 |
| 3102 | O | SER B | 8 | 38.019 | 60.525 | 11.594 | 1.00 | 61.68 |
| 3103 | N | LEU B | 9 | 39.092 | 62.435 | 12.106 | 1.00 | 91.60 |
| 3104 | CA | LEU B | 9 | 39.699 | 62.594 | 10.790 | 1.00 | 91.60 |
| 3105 | CB | LEU B | 9 | 39.080 | 63.779 | 10.053 | 1.00 | 67.13 |
| 3106 | CG | LEU B | 9 | 37.601 | 63.806 | 9.688 | 1.00 | 67.13 |
| 3107 | CD1 | LEU B | 9 | 37.378 | 64.735 | 8.524 | 1.00 | 67.13 |
| 3108 | CD2 | LEU B | 9 | 37.167 | 62.435 | 9.291 | 1.00 | 67.13 |
| 3109 | C | LEU B | 9 | 41.195 | 62.847 | 10.897 | 1.00 | 91.60 |
| 3110 | O | LEU B | 9 | 41.675 | 63.347 | 11.915 | 1.00 | 91.60 |
| 3111 | N | ASN B | 10 | 41.928 | 62.519 | 9.835 | 1.00 | 84.59 |
| 3112 | CA | ASN B | 10 | 43.369 | 62.742 | 9.800 | 1.00 | 84.59 |
| 3113 | CB | ASN B | 10 | 44.107 | 61.640 | 10.548 | 1.00 | 140.61 |
| 3114 | CG | ASN B | 10 | 45.558 | 61.978 | 10.763 | 1.00 | 140.61 |
| 3115 | OD1 | ASN B | 10 | 45.889 | 62.905 | 11.505 | 1.00 | 140.61 |
| 3116 | ND2 | ASN B | 10 | 46.438 | 61.241 | 10.099 | 1.00 | 140.61 |
| 3117 | C | ASN B | 10 | 43.876 | 62.812 | 8.367 | 1.00 | 84.59 |
| 3118 | O | ASN B | 10 | 43.883 | 61.805 | 7.656 | 1.00 | 84.59 |
| 3119 | N | PRO B | 11 | 44.310 | 64.010 | 7.917 | 1.00 | 77.61 |
| 3120 | CD | PRO B | 11 | 44.699 | 64.185 | 6.506 | 1.00 | 115.85 |
| 3121 | CA | PRO B | 11 | 44.370 | 65.289 | 8.638 | 1.00 | 77.61 |
| 3122 | CB | PRO B | 11 | 44.811 | 66.264 | 7.544 | 1.00 | 115.85 |
| 3123 | CG | PRO B | 11 | 45.560 | 65.394 | 6.570 | 1.00 | 115.85 |
| 3124 | C | PRO B | 11 | 43.042 | 65.731 | 9.290 | 1.00 | 77.61 |
| 3125 | O | PRO B | 11 | 41.982 | 65.231 | 8.937 | 1.00 | 77.61 |
| 3126 | N | PRO B | 12 | 43.085 | 66.680 | 10.239 | 1.00 | 88.06 |
| 3127 | CD | PRO B | 12 | 44.279 | 67.378 | 10.753 | 1.00 | 174.82 |
| 3128 | CA | PRO B | 12 | 41.883 | 67.170 | 10.921 | 1.00 | 88.06 |
| 3129 | CB | PRO B | 12 | 42.433 | 68.119 | 11.982 | 1.00 | 174.82 |
| 3130 | CG | PRO B | 12 | 43.854 | 67.699 | 12.148 | 1.00 | 174.82 |
| 3131 | C | PRO B | 12 | 40.993 | 67.939 | 9.924 | 1.00 | 88.06 |
| 3132 | O | PRO B | 12 | 39.781 | 68.071 | 10.108 | 1.00 | 88.06 |
| 3133 | N | TRP B | 13 | 41.623 | 68.464 | 8.880 | 1.00 | 96.43 |
| 3134 | CA | TRP B | 13 | 40.932 | 69.239 | 7.859 | 1.00 | 96.43 |
| 3135 | CB | TRP B | 13 | 41.907 | 69.605 | 6.731 | 1.00 | 96.49 |
| 3136 | CG | TRP B | 13 | 43.190 | 70.134 | 7.232 | 1.00 | 96.49 |
| 3137 | CD2 | TRP B | 13 | 43.376 | 70.974 | 8.358 | 1.00 | 96.49 |
| 3138 | CE2 | TRP B | 13 | 44.757 | 71.168 | 8.509 | 1.00 | 96.49 |
| 3139 | CE3 | TRP B | 13 | 42.504 | 71.588 | 9.268 | 1.00 | 96.49 |
| 3140 | CD1 | TRP B | 13 | 44.423 | 69.863 | 6.745 | 1.00 | 96.49 |
| 3141 | NE1 | TRP B | 13 | 45.373 | 70.474 | 7.506 | 1.00 | 96.49 |
| 3142 | CZ2 | TRP B | 13 | 45.298 | 71.947 | 9.532 | 1.00 | 96.49 |
| 3143 | CZ3 | TRP B | 13 | 43.034 | 72.363 | 10.283 | 1.00 | 96.49 |
| 3144 | CH2 | TRP B | 13 | 44.424 | 72.536 | 10.410 | 1.00 | 96.49 |
| 3145 | C | TRP B | 13 | 39.742 | 68.497 | 7.281 | 1.00 | 96.43 |
| 3146 | O | TRP B | 13 | 39.882 | 67.403 | 6.738 | 1.00 | 96.43 |
| 3147 | N | ASN B | 14 | 38.567 | 69.102 | 7.407 | 1.00 | 72.14 |
| 3148 | CA | ASN B | 14 | 37.352 | 68.509 | 6.867 | 1.00 | 72.14 |
| 3149 | CB | ASN B | 14 | 36.239 | 68.455 | 7.931 | 1.00 | 117.87 |
| 3150 | CG | ASN B | 14 | 35.712 | 69.812 | 8.309 | 1.00 | 117.87 |
| 3151 | OD1 | ASN B | 14 | 36.462 | 70.695 | 8.718 | 1.00 | 117.87 |
| 3152 | ND2 | ASN B | 14 | 34.407 | 69.984 | 8.184 | 1.00 | 117.87 |
| 3153 | C | ASN B | 14 | 36.858 | 69.201 | 5.588 | 1.00 | 72.14 |
| 3154 | O | ASN B | 14 | 35.721 | 69.018 | 5.177 | 1.00 | 72.14 |
| 3155 | N | ARG B | 15 | 37.715 | 70.009 | 4.973 | 1.00 | 61.00 |
| 3156 | CA | ARG B | 15 | 37.399 | 70.653 | 3.701 | 1.00 | 61.00 |
| 3157 | CB | ARG B | 15 | 37.241 | 72.149 | 3.841 | 1.00 | 68.74 |
| 3158 | CG | ARG B | 15 | 36.513 | 72.569 | 5.064 | 1.00 | 68.74 |
| 3159 | CD | ARG B | 15 | 36.354 | 74.075 | 5.045 | 1.00 | 68.74 |
| 3160 | NE | ARG B | 15 | 35.436 | 74.525 | 4.007 | 1.00 | 68.74 |
| 3161 | CZ | ARG B | 15 | 35.531 | 75.714 | 3.429 | 1.00 | 68.74 |
| 3162 | NH1 | ARG B | 15 | 36.501 | 76.533 | 3.794 | 1.00 | 68.74 |
| 3163 | NH2 | ARG B | 15 | 34.660 | 76.093 | 2.498 | 1.00 | 68.74 |
| 3164 | C | ARG B | 15 | 38.662 | 70.393 | 2.900 | 1.00 | 61.00 |
| 3165 | O | ARG B | 15 | 39.707 | 70.950 | 3.199 | 1.00 | 61.00 |
| 3166 | N | ILE B | 16 | 38.587 | 69.540 | 1.895 | 1.00 | 73.69 |
| 3167 | CA | ILE B | 16 | 39.770 | 69.256 | 1.135 | 1.00 | 73.69 |
| 3168 | CB | ILE B | 16 | 40.194 | 67.833 | 1.339 | 1.00 | 63.86 |
| 3169 | CG2 | ILE B | 16 | 40.624 | 67.645 | 2.767 | 1.00 | 63.86 |
| 3170 | CG1 | ILE B | 16 | 39.044 | 66.895 | 0.994 | 1.00 | 63.86 |
| 3171 | CD1 | ILE B | 16 | 39.388 | 65.448 | 1.178 | 1.00 | 63.86 |
| 3172 | C | ILE B | 16 | 39.621 | 69.493 | −0.340 | 1.00 | 73.69 |
| 3173 | O | ILE B | 16 | 38.516 | 69.651 | −0.866 | 1.00 | 73.69 |
| 3174 | N | PHE B | 17 | 40.770 | 69.491 | −0.998 | 1.00 | 99.56 |
| 3175 | CA | PHE B | 17 | 40.889 | 69.696 | −2.425 | 1.00 | 99.56 |
| 3176 | CB | PHE B | 17 | 42.282 | 70.211 | −2.720 | 1.00 | 81.03 |
| 3177 | CG | PHE B | 17 | 42.400 | 71.703 | −2.699 | 1.00 | 81.03 |
| 3178 | CD1 | PHE B | 17 | 43.515 | 72.315 | −2.128 | 1.00 | 81.03 |
| 3179 | CD2 | PHE B | 17 | 41.453 | 72.497 | −3.344 | 1.00 | 81.03 |
| 3180 | CE1 | PHE B | 17 | 43.685 | 73.688 | −2.204 | 1.00 | 81.03 |
| 3181 | CE2 | PHE B | 17 | 41.613 | 73.877 | −3.428 | 1.00 | 81.03 |
| 3182 | CZ | PHE B | 17 | 42.733 | 74.475 | −2.860 | 1.00 | 81.03 |
| 3183 | C | PHE B | 17 | 40.678 | 68.392 | −3.169 | 1.00 | 99.56 |
| 3184 | O | PHE B | 17 | 40.804 | 67.321 | −2.591 | 1.00 | 99.56 |
| 3185 | N | LYS B | 18 | 40.374 | 68.484 | −4.459 | 1.00 | 100.47 |
| 3186 | CA | LYS B | 18 | 40.160 | 67.302 | −5.288 | 1.00 | 100.47 |
| 3187 | CB | LYS B | 18 | 39.700 | 67.733 | −6.682 | 1.00 | 201.96 |
| 3188 | CG | LYS B | 18 | 39.302 | 66.601 | −7.612 | 1.00 | 201.96 |
| 3189 | CD | LYS B | 18 | 38.552 | 67.166 | −8.809 | 1.00 | 201.96 |
| 3190 | CE | LYS B | 18 | 38.122 | 66.086 | −9.776 | 1.00 | 201.96 |
| 3191 | NZ | LYS B | 18 | 39.299 | 65.382 | −10.345 | 1.00 | 201.96 |
| 3192 | C | LYS B | 18 | 41.448 | 66.492 | −5.394 | 1.00 | 100.47 |
| 3193 | O | LYS B | 18 | 42.518 | 67.028 | −5.671 | 1.00 | 100.47 |
| 3194 | N | GLY B | 19 | 41.362 | 65.197 | −5.143 | 1.00 | 65.25 |
| 3195 | CA | GLY B | 19 | 42.547 | 64.371 | −5.264 | 1.00 | 85.25 |
| 3196 | C | GLY B | 19 | 43.350 | 64.115 | −4.008 | 1.00 | 85.25 |
| 3197 | O | GLY B | 19 | 44.237 | 63.274 | −3.996 | 1.00 | 85.25 |
| 3198 | N | GLU B | 20 | 43.057 | 64.825 | −2.937 | 1.00 | 70.09 |
| 3199 | CA | GLU B | 20 | 43.804 | 64.606 | −1.701 | 1.00 | 70.09 |
| 3200 | CB | GLU B | 20 | 43.685 | 65.846 | −0.813 | 1.00 | 167.13 |
| 3201 | CG | GLU B | 20 | 44.020 | 67.133 | −1.566 | 1.00 | 167.13 |
| 3202 | CD | GLU B | 20 | 44.034 | 68.357 | −0.677 | 1.00 | 167.13 |
| 3203 | OE1 | GLU B | 20 | 43.009 | 68.622 | −0.013 | 1.00 | 167.13 |
| 3204 | OE2 | GLU B | 20 | 45.070 | 69.056 | −0.655 | 1.00 | 167.13 |
| 3205 | C | GLU B | 20 | 43.296 | 63.356 | −0.967 | 1.00 | 70.09 |
| 3206 | O | GLU B | 20 | 42.273 | 62.769 | −1.368 | 1.00 | 70.09 |
| 3207 | N | ASN B | 21 | 44.002 | 62.935 | 0.086 | 1.00 | 77.36 |
| 3208 | CA | ASN B | 21 | 43.579 | 61.747 | 0.830 | 1.00 | 77.36 |
| 3209 | CB | ASN B | 21 | 44.626 | 60.630 | 0.802 | 1.00 | 155.50 |
| 3210 | CG | ASN B | 21 | 45.285 | 60.472 | −0.537 | 1.00 | 155.50 |
| 3211 | OD1 | ASN B | 21 | 44.634 | 60.520 | −1.585 | 1.00 | 155.50 |
| 3212 | ND2 | ASN B | 21 | 46.598 | 60.265 | −0.490 | 1.00 | 155.50 |
| 3213 | C | ASN B | 21 | 43.300 | 62.066 | 2.287 | 1.00 | 77.36 |
| 3214 | O | ASN B | 21 | 43.997 | 62.877 | 2.892 | 1.00 | 77.36 |
| 3215 | N | VAL B | 22 | 42.286 | 61.409 | 2.848 | 1.00 | 68.07 |
| 3216 | CA | VAL B | 22 | 41.899 | 61.602 | 4.241 | 1.00 | 68.07 |
| 3217 | CB | VAL B | 22 | 40.732 | 62.572 | 4.364 | 1.00 | 74.66 |
| 3218 | CG1 | VAL B | 22 | 39.514 | 62.023 | 3.658 | 1.00 | 74.66 |
| 3219 | CG2 | VAL B | 22 | 40.438 | 62.807 | 5.811 | 1.00 | 74.66 |
| 3220 | C | VAL B | 22 | 41.469 | 60.270 | 4.829 | 1.00 | 68.07 |
| 3221 | O | VAL B | 22 | 40.964 | 59.391 | 4.120 | 1.00 | 68.07 |
| 3222 | N | THR B | 23 | 41.646 | 60.123 | 6.132 | 1.00 | 73.02 |
| 3223 | CA | THR B | 23 | 41.316 | 58.865 | 6.791 | 1.00 | 73.02 |
| 3224 | CB | THR B | 23 | 42.576 | 58.274 | 7.428 | 1.00 | 107.36 |
| 3225 | OG1 | THR B | 23 | 43.602 | 58.155 | 6.435 | 1.00 | 107.36 |
| 3226 | CG2 | THR B | 23 | 42.288 | 56.919 | 8.018 | 1.00 | 107.36 |
| 3227 | C | THR B | 23 | 40.278 | 59.057 | 7.885 | 1.00 | 73.02 |
| 3228 | O | THR B | 23 | 40.446 | 59.918 | 8.739 | 1.00 | 73.02 |
| 3229 | N | LEU B | 24 | 39.211 | 58.261 | 7.888 | 1.00 | 82.14 |
| 3230 | CA | LEU B | 24 | 38.180 | 58.423 | 8.920 | 1.00 | 82.14 |
| 3231 | CB | LEU B | 24 | 36.771 | 58.497 | 8.323 | 1.00 | 67.63 |
| 3232 | CG | LEU B | 24 | 36.534 | 59.233 | 6.996 | 1.00 | 67.63 |
| 3233 | CD1 | LEU B | 24 | 35.063 | 59.448 | 6.787 | 1.00 | 67.63 |
| 3234 | CD2 | LEU B | 24 | 37.249 | 60.554 | 6.987 | 1.00 | 67.63 |
| 3235 | C | LEU B | 24 | 38.205 | 57.286 | 9.904 | 1.00 | 82.14 |
| 3236 | O | LEU B | 24 | 37.732 | 56.195 | 9.615 | 1.00 | 82.14 |
| 3237 | N | THR B | 25 | 38.735 | 57.551 | 11.086 | 1.00 | 78.19 |
| 3238 | CA | THR B | 25 | 38.817 | 56.519 | 12.099 | 1.00 | 78.19 |
| 3239 | CB | THR B | 25 | 40.047 | 56.755 | 12.971 | 1.00 | 154.05 |
| 3240 | OG1 | THR B | 25 | 41.200 | 56.846 | 12.124 | 1.00 | 154.05 |
| 3241 | CG2 | THR B | 25 | 40.231 | 55.618 | 13.949 | 1.00 | 154.05 |
| 3242 | C | THR B | 25 | 37.554 | 56.489 | 12.941 | 1.00 | 78.19 |
| 3243 | O | THR B | 25 | 37.022 | 57.532 | 13.310 | 1.00 | 78.19 |
| 3244 | N | CYS B | 26 | 37.044 | 55.296 | 13.210 | 1.00 | 82.94 |
| 3245 | CA | CYS B | 26 | 35.860 | 55.184 | 14.049 | 1.00 | 82.94 |
| 3246 | C | CYS B | 26 | 36.280 | 55.187 | 15.510 | 1.00 | 82.94 |
| 3247 | O | CYS B | 26 | 37.254 | 54.549 | 15.896 | 1.00 | 82.94 |
| 3248 | CB | CYS B | 26 | 35.094 | 53.908 | 13.763 | 1.00 | 125.46 |
| 3249 | SG | CYS B | 26 | 33.481 | 53.869 | 14.604 | 1.00 | 125.46 |
| 3250 | N | ASN B | 27 | 35.535 | 55.931 | 16.309 | 1.00 | 247.26 |
| 3251 | CA | ASN B | 27 | 35.784 | 56.058 | 17.730 | 1.00 | 247.26 |
| 3252 | CB | ASN B | 27 | 34.500 | 55.740 | 18.470 | 1.00 | 240.69 |
| 3253 | CG | ASN B | 27 | 34.506 | 56.280 | 19.864 | 1.00 | 240.69 |
| 3254 | OD1 | ASN B | 27 | 35.033 | 57.371 | 20.107 | 1.00 | 240.69 |
| 3255 | ND2 | ASN B | 27 | 33.909 | 55.541 | 20.797 | 1.00 | 240.69 |
| 3256 | C | ASN B | 27 | 36.922 | 55.201 | 18.289 | 1.00 | 247.26 |
| 3257 | O | ASN B | 27 | 36.702 | 54.072 | 18.722 | 1.00 | 247.26 |
| 3258 | N | GLY B | 28 | 38.135 | 55.748 | 18.286 | 1.00 | 195.90 |
| 3259 | CA | GLY B | 28 | 39.286 | 55.021 | 18.792 | 1.00 | 195.90 |
| 3260 | C | GLY B | 28 | 40.518 | 55.841 | 18.506 | 1.00 | 195.90 |
| 3261 | O | GLY B | 28 | 40.788 | 56.161 | 17.355 | 1.00 | 195.90 |
| 3262 | N | ASN B | 29 | 41.274 | 56.181 | 19.541 | 1.00 | 230.48 |
| 3263 | CA | ASN B | 29 | 42.456 | 57.003 | 19.352 | 1.00 | 230.48 |
| 3264 | CB | ASN B | 29 | 42.882 | 57.612 | 20.690 | 1.00 | 249.51 |
| 3265 | CG | ASN B | 29 | 43.919 | 58.710 | 20.527 | 1.00 | 249.51 |
| 3266 | OD1 | ASN B | 29 | 44.154 | 59.198 | 19.421 | 1.00 | 249.51 |
| 3267 | ND2 | ASN B | 29 | 44.534 | 59.115 | 21.634 | 1.00 | 249.51 |
| 3268 | C | ASN B | 29 | 43.644 | 56.302 | 18.699 | 1.00 | 230.48 |
| 3269 | O | ASN B | 29 | 44.198 | 56.804 | 17.716 | 1.00 | 230.48 |
| 3270 | N | ASN B | 30 | 44.040 | 55.149 | 19.229 | 1.00 | 218.47 |
| 3271 | CA | ASN B | 30 | 45.185 | 54.441 | 18.667 | 1.00 | 218.47 |
| 3272 | CB | ASN B | 30 | 46.364 | 54.509 | 19.631 | 1.00 | 238.45 |
| 3273 | CG | ASN B | 30 | 46.841 | 55.909 | 19.854 | 1.00 | 238.45 |
| 3274 | OD1 | ASN B | 30 | 46.936 | 56.337 | 20.986 | 1.00 | 238.45 |
| 3275 | ND2 | ASN B | 30 | 47.139 | 56.635 | 18.778 | 1.00 | 238.45 |
| 3276 | C | ASN B | 30 | 44.936 | 52.990 | 18.293 | 1.00 | 218.47 |
| 3277 | O | ASN B | 30 | 44.881 | 52.646 | 17.109 | 1.00 | 218.47 |
| 3278 | N | PHE B | 31 | 44.779 | 52.137 | 19.300 | 1.00 | 249.37 |
| 3279 | CA | PHE B | 31 | 44.573 | 50.724 | 19.037 | 1.00 | 249.37 |
| 3280 | CB | PHE B | 31 | 45.620 | 49.901 | 19.799 | 1.00 | 234.42 |
| 3281 | CG | PHE B | 31 | 47.045 | 50.311 | 19.513 | 1.00 | 234.42 |
| 3282 | CD1 | PHE B | 31 | 47.600 | 51.429 | 20.131 | 1.00 | 234.42 |
| 3283 | CD2 | PHE B | 31 | 47.822 | 49.594 | 18.607 | 1.00 | 234.42 |
| 3284 | CE1 | PHE B | 31 | 48.909 | 51.828 | 19.854 | 1.00 | 234.42 |
| 3285 | CE2 | PHE B | 31 | 49.131 | 49.985 | 18.322 | 1.00 | 234.42 |
| 3286 | CZ | PHE B | 31 | 49.674 | 51.107 | 18.947 | 1.00 | 234.42 |
| 3287 | C | PHE B | 31 | 43.166 | 50.221 | 19.340 | 1.00 | 249.37 |
| 3288 | O | PHE B | 31 | 42.638 | 50.399 | 20.440 | 1.00 | 249.37 |
| 3289 | N | PHE B | 32 | 42.579 | 49.581 | 18.332 | 1.00 | 162.47 |
| 3290 | CA | PHE B | 32 | 41.233 | 49.034 | 18.408 | 1.00 | 162.47 |
| 3291 | CB | PHE B | 32 | 40.337 | 49.732 | 17.386 | 1.00 | 249.69 |
| 3292 | CG | PHE B | 32 | 38.872 | 49.480 | 17.598 | 1.00 | 249.69 |
| 3293 | CD1 | PHE B | 32 | 38.241 | 49.969 | 18.718 | 1.00 | 249.69 |
| 3294 | CD2 | PHE B | 32 | 38.130 | 48.733 | 16.690 | 1.00 | 249.69 |
| 3295 | CE1 | PHE B | 32 | 36.894 | 49.721 | 18.931 | 1.00 | 249.69 |
| 3296 | CE2 | PHE B | 32 | 36.772 | 48.482 | 16.901 | 1.00 | 249.69 |
| 3297 | CZ | PHE B | 32 | 36.170 | 48.988 | 18.039 | 1.00 | 249.69 |
| 3298 | C | PHE B | 32 | 41.243 | 47.533 | 18.118 | 1.00 | 162.47 |
| 3299 | O | PHE B | 32 | 42.275 | 46.987 | 17.714 | 1.00 | 162.47 |
| 3300 | N | GLU B | 33 | 40.097 | 46.869 | 18.298 | 1.00 | 249.28 |
| 3301 | CA | GLU B | 33 | 40.035 | 45.425 | 18.046 | 1.00 | 249.28 |
| 3302 | CB | GLU B | 33 | 39.767 | 44.643 | 19.298 | 1.00 | 249.25 |
| 3303 | CG | GLU B | 33 | 39.978 | 43.139 | 19.158 | 1.00 | 249.25 |
| 3304 | CD | GLU B | 33 | 41.395 | 42.776 | 18.727 | 1.00 | 249.25 |
| 3305 | OE1 | GLU B | 33 | 42.373 | 43.401 | 19.192 | 1.00 | 249.25 |
| 3306 | OE2 | GLU B | 33 | 41.565 | 41.829 | 17.938 | 1.00 | 249.25 |
| 3307 | C | GLU B | 33 | 38.993 | 44.918 | 17.083 | 1.00 | 249.28 |
| 3308 | O | GLU B | 33 | 39.312 | 44.164 | 16.171 | 1.00 | 249.28 |
| 3309 | N | VAL B | 34 | 37.732 | 45.243 | 17.348 | 1.00 | 234.23 |
| 3310 | CA | VAL B | 34 | 36.657 | 44.756 | 16.507 | 1.00 | 234.23 |
| 3311 | CB | VAL B | 34 | 35.301 | 45.388 | 16.902 | 1.00 | 191.69 |
| 3312 | CG1 | VAL B | 34 | 34.197 | 44.865 | 15.998 | 1.00 | 191.69 |
| 3313 | CG2 | VAL B | 34 | 34.978 | 45.059 | 18.343 | 1.00 | 191.69 |
| 3314 | C | VAL B | 34 | 36.919 | 44.972 | 15.029 | 1.00 | 234.23 |
| 3315 | O | VAL B | 34 | 37.592 | 45.923 | 14.632 | 1.00 | 234.23 |
| 3316 | N | SER B | 35 | 36.395 | 44.052 | 14.229 | 1.00 | 249.39 |
| 3317 | CA | SER B | 35 | 36.536 | 44.110 | 12.789 | 1.00 | 249.39 |
| 3318 | CB | SER B | 35 | 37.053 | 42.775 | 12.246 | 1.00 | 187.29 |
| 3319 | OG | SER B | 35 | 36.078 | 41.759 | 12.404 | 1.00 | 187.29 |
| 3320 | C | SER B | 35 | 35.161 | 44.414 | 12.202 | 1.00 | 249.39 |
| 3321 | O | SER B | 35 | 35.008 | 44.523 | 10.988 | 1.00 | 249.39 |
| 3322 | N | SER B | 36 | 34.160 | 44.541 | 13.074 | 1.00 | 236.03 |
| 3323 | CA | SER B | 36 | 32.796 | 44.846 | 12.641 | 1.00 | 236.03 |
| 3324 | CB | SER B | 36 | 31.770 | 43.967 | 13.369 | 1.00 | 174.69 |
| 3325 | OG | SER B | 36 | 31.663 | 44.323 | 14.735 | 1.00 | 174.69 |
| 3326 | C | SER B | 36 | 32.488 | 46.310 | 12.911 | 1.00 | 236.03 |
| 3327 | O | SER B | 36 | 32.037 | 46.684 | 13.992 | 1.00 | 236.03 |
| 3328 | N | THR B | 37 | 32.752 | 47.137 | 11.910 | 1.00 | 186.56 |
| 3329 | CA | THR B | 37 | 32.516 | 48.565 | 11.996 | 1.00 | 186.56 |
| 3330 | CB | THR B | 37 | 33.852 | 49.349 | 11.926 | 1.00 | 204.69 |
| 3331 | OG1 | THR B | 37 | 34.720 | 48.919 | 12.983 | 1.00 | 204.69 |
| 3332 | CG2 | THR B | 37 | 33.611 | 50.838 | 12.065 | 1.00 | 204.69 |
| 3333 | C | THR B | 37 | 31.649 | 48.899 | 10.789 | 1.00 | 186.56 |
| 3334 | O | THR B | 37 | 31.837 | 48.338 | 9.708 | 1.00 | 186.56 |
| 3335 | N | LYS B | 38 | 30.692 | 49.800 | 10.972 | 1.00 | 233.53 |
| 3336 | CA | LYS B | 38 | 29.803 | 50.182 | 9.883 | 1.00 | 233.53 |
| 3337 | CB | LYS B | 38 | 28.358 | 50.059 | 10.341 | 1.00 | 159.29 |
| 3338 | CG | LYS B | 38 | 28.005 | 48.688 | 10.851 | 1.00 | 159.29 |
| 3339 | CD | LYS B | 38 | 26.556 | 48.633 | 11.299 | 1.00 | 159.29 |
| 3340 | CE | LYS B | 38 | 26.179 | 47.229 | 11.738 | 1.00 | 159.29 |
| 3341 | NZ | LYS B | 38 | 24.755 | 47.156 | 12.158 | 1.00 | 159.29 |
| 3342 | C | LYS B | 38 | 30.055 | 51.604 | 9.402 | 1.00 | 233.53 |
| 3343 | O | LYS B | 38 | 30.349 | 52.490 | 10.203 | 1.00 | 233.53 |
| 3344 | N | TRP B | 39 | 29.936 | 51.818 | 8.092 | 1.00 | 87.42 |
| 3345 | CA | TRP B | 39 | 30.140 | 53.149 | 7.521 | 1.00 | 87.42 |
| 3346 | CB | TRP B | 39 | 31.422 | 53.229 | 6.688 | 1.00 | 107.80 |
| 3347 | CG | TRP B | 39 | 32.678 | 53.035 | 7.471 | 1.00 | 107.80 |
| 3348 | CD2 | TRP B | 39 | 33.240 | 53.923 | 8.438 | 1.00 | 107.80 |
| 3349 | CE2 | TRP B | 39 | 34.427 | 53.326 | 8.904 | 1.00 | 107.80 |
| 3350 | CE3 | TRP B | 39 | 32.857 | 55.160 | 8.954 | 1.00 | 107.80 |
| 3351 | CD1 | TRP B | 39 | 33.521 | 51.968 | 7.395 | 1.00 | 107.80 |
| 3352 | NE1 | TRP B | 39 | 34.574 | 52.135 | 8.253 | 1.00 | 107.80 |
| 3353 | CZ2 | TRP B | 39 | 35.236 | 53.930 | 9.860 | 1.00 | 107.80 |
| 3354 | CZ3 | TRP B | 39 | 33.659 | 55.755 | 9.899 | 1.00 | 107.80 |
| 3355 | CH2 | TRP B | 39 | 34.839 | 55.141 | 10.346 | 1.00 | 107.80 |
| 3356 | C | TRP B | 39 | 28.973 | 53.500 | 6.637 | 1.00 | 87.42 |
| 3357 | O | TRP B | 39 | 28.580 | 52.712 | 5.799 | 1.00 | 87.42 |
| 3358 | N | PHE B | 40 | 28.429 | 54.694 | 6.818 | 1.00 | 127.18 |
| 3359 | CA | PHE B | 40 | 27.289 | 55.111 | 6.025 | 1.00 | 127.18 |
| 3360 | CB | PHE B | 40 | 26.052 | 55.264 | 6.908 | 1.00 | 155.57 |
| 3361 | CG | PHE B | 40 | 25.695 | 54.032 | 7.687 | 1.00 | 155.57 |
| 3362 | CD1 | PHE B | 40 | 26.374 | 53.715 | 8.858 | 1.00 | 155.57 |
| 3363 | CD2 | PHE B | 40 | 24.666 | 53.200 | 7.261 | 1.00 | 155.57 |
| 3364 | CE1 | PHE B | 40 | 26.024 | 52.589 | 9.600 | 1.00 | 155.57 |
| 3365 | CE2 | PHE B | 40 | 24.308 | 52.074 | 7.991 | 1.00 | 155.57 |
| 3366 | CZ | PHE B | 40 | 24.987 | 51.764 | 9.162 | 1.00 | 155.57 |
| 3367 | C | PHE B | 40 | 27.523 | 56.414 | 5.281 | 1.00 | 127.18 |
| 3368 | O | PHE B | 40 | 27.208 | 57.495 | 5.773 | 1.00 | 127.18 |
| 3369 | N | HIS B | 41 | 28.078 | 56.306 | 4.084 | 1.00 | 72.05 |
| 3370 | CA | HIS B | 41 | 28.329 | 57.484 | 3.260 | 1.00 | 72.05 |
| 3371 | CB | HIS B | 41 | 29.355 | 57.132 | 2.173 | 1.00 | 83.13 |
| 3372 | CG | HIS B | 41 | 29.650 | 58.256 | 1.230 | 1.00 | 83.13 |
| 3373 | CD2 | HIS B | 41 | 29.801 | 58.274 | −0.114 | 1.00 | 83.13 |
| 3374 | ND1 | HIS B | 41 | 29.837 | 59.553 | 1.656 | 1.00 | 83.13 |
| 3375 | CE1 | HIS B | 41 | 30.087 | 60.323 | 0.614 | 1.00 | 83.13 |
| 3376 | NE2 | HIS B | 41 | 30.071 | 59.571 | −0.472 | 1.00 | 83.13 |
| 3377 | C | HIS B | 41 | 27.010 | 57.961 | 2.633 | 1.00 | 72.05 |
| 3378 | O | HIS B | 41 | 26.458 | 57.298 | 1.761 | 1.00 | 72.05 |
| 3379 | N | ASN B | 42 | 26.527 | 59.123 | 3.069 | 1.00 | 104.44 |
| 3380 | CA | ASN B | 42 | 25.256 | 59.683 | 2.600 | 1.00 | 104.44 |
| 3381 | CB | ASN B | 42 | 25.240 | 59.870 | 1.077 | 1.00 | 64.53 |
| 3382 | CG | ASN B | 42 | 26.091 | 61.039 | 0.625 | 1.00 | 64.53 |
| 3383 | OD1 | ASN B | 42 | 27.195 | 61.213 | 1.144 | 1.00 | 64.53 |
| 3384 | ND2 | ASN B | 42 | 25.618 | 61.828 | −0.348 | 1.00 | 64.53 |
| 3385 | C | ASN B | 42 | 24.114 | 58.751 | 2.999 | 1.00 | 104.44 |
| 3386 | O | ASN B | 42 | 23.089 | 58.706 | 2.334 | 1.00 | 104.44 |
| 3387 | N | GLY B | 43 | 24.293 | 58.003 | 4.083 | 1.00 | 163.92 |
| 3388 | CA | GLY B | 43 | 23.246 | 57.092 | 4.522 | 1.00 | 163.92 |
| 3389 | C | GLY B | 43 | 23.405 | 55.677 | 3.991 | 1.00 | 163.92 |
| 3390 | O | GLY B | 43 | 23.159 | 54.701 | 4.702 | 1.00 | 163.92 |
| 3391 | N | SER B | 44 | 23.816 | 55.562 | 2.735 | 1.00 | 175.12 |
| 3392 | CA | SER B | 44 | 24.017 | 54.262 | 2.106 | 1.00 | 175.12 |
| 3393 | CB | SER B | 44 | 24.326 | 54.445 | 0.620 | 1.00 | 173.04 |
| 3394 | OG | SER B | 44 | 23.344 | 55.253 | −0.002 | 1.00 | 173.04 |
| 3395 | C | SER B | 44 | 25.178 | 53.524 | 2.772 | 1.00 | 175.12 |
| 3396 | O | SER B | 44 | 26.275 | 54.070 | 2.899 | 1.00 | 175.12 |
| 3397 | N | LEU B | 45 | 24.944 | 52.285 | 3.197 | 1.00 | 151.43 |
| 3398 | CA | LEU B | 45 | 25.991 | 51.495 | 3.846 | 1.00 | 151.43 |
| 3399 | CB | LEU B | 45 | 25.458 | 50.101 | 4.198 | 1.00 | 163.91 |
| 3400 | CG | LEU B | 45 | 26.424 | 49.160 | 4.922 | 1.00 | 163.91 |
| 3401 | CD1 | LEU B | 45 | 26.972 | 49.825 | 6.176 | 1.00 | 163.91 |
| 3402 | CD2 | LEU B | 45 | 25.701 | 47.872 | 5.275 | 1.00 | 163.91 |
| 3403 | C | LEU B | 45 | 27.220 | 51.376 | 2.944 | 1.00 | 151.43 |
| 3404 | O | LEU B | 45 | 27.089 | 51.318 | 1.722 | 1.00 | 151.43 |
| 3405 | N | SER B | 46 | 28.411 | 51.350 | 3.541 | 1.00 | 127.11 |
| 3406 | CA | SER B | 46 | 29.646 | 51.241 | 2.770 | 1.00 | 127.11 |
| 3407 | CB | SER B | 46 | 30.724 | 52.142 | 3.366 | 1.00 | 226.86 |
| 3408 | OG | SER B | 46 | 31.902 | 52.103 | 2.574 | 1.00 | 226.86 |
| 3409 | C | SER B | 46 | 30.103 | 49.791 | 2.810 | 1.00 | 127.11 |
| 3410 | O | SER B | 46 | 29.622 | 49.009 | 3.626 | 1.00 | 127.11 |
| 3411 | N | GLU B | 47 | 31.030 | 49.425 | 1.927 | 1.00 | 149.05 |
| 3412 | CA | GLU B | 47 | 31.486 | 48.041 | 1.929 | 1.00 | 149.05 |
| 3413 | CB | GLU B | 47 | 31.711 | 47.509 | 0.484 | 1.00 | 195.89 |
| 3414 | CG | GLU B | 47 | 30.777 | 48.077 | −0.608 | 1.00 | 195.89 |
| 3415 | CD | GLU B | 47 | 31.343 | 47.934 | −2.030 | 1.00 | 195.89 |
| 3416 | OE1 | GLU B | 47 | 32.086 | 48.832 | −2.508 | 1.00 | 195.89 |
| 3417 | OE2 | GLU B | 47 | 31.042 | 46.909 | −2.685 | 1.00 | 195.89 |
| 3418 | C | GLU B | 47 | 32.738 | 47.807 | 2.808 | 1.00 | 149.05 |
| 3419 | O | GLU B | 47 | 33.224 | 46.684 | 2.891 | 1.00 | 149.05 |
| 3420 | N | GLU B | 48 | 33.291 | 48.851 | 3.436 | 1.00 | 101.79 |
| 3421 | CA | GLU B | 48 | 34.458 | 48.628 | 4.299 | 1.00 | 101.79 |
| 3422 | CB | GLU B | 48 | 35.331 | 49.904 | 4.457 | 1.00 | 223.78 |
| 3423 | CG | GLU B | 48 | 36.479 | 49.790 | 5.499 | 1.00 | 223.78 |
| 3424 | CD | GLU B | 48 | 37.584 | 48.801 | 5.127 | 1.00 | 223.78 |
| 3425 | OE1 | GLU B | 48 | 38.340 | 49.074 | 4.170 | 1.00 | 223.78 |
| 3426 | OE2 | GLU B | 48 | 37.703 | 47.751 | 5.799 | 1.00 | 223.78 |
| 3427 | C | GLU B | 48 | 33.949 | 48.158 | 5.661 | 1.00 | 101.79 |
| 3428 | O | GLU B | 48 | 32.788 | 48.397 | 6.021 | 1.00 | 101.79 |
| 3429 | N | THR B | 49 | 34.812 | 47.476 | 6.410 | 1.00 | 169.38 |
| 3430 | CA | THR B | 49 | 34.445 | 46.976 | 7.728 | 1.00 | 169.38 |
| 3431 | CB | THR B | 49 | 34.268 | 45.441 | 7.707 | 1.00 | 162.45 |
| 3432 | OG1 | THR B | 49 | 35.467 | 44.824 | 7.222 | 1.00 | 162.45 |
| 3433 | CG2 | THR B | 49 | 33.110 | 45.061 | 6.797 | 1.00 | 162.45 |
| 3434 | C | THR B | 49 | 35.501 | 47.369 | 8.762 | 1.00 | 169.38 |
| 3435 | O | THR B | 49 | 35.190 | 47.530 | 9.940 | 1.00 | 169.38 |
| 3436 | N | ASN B | 50 | 36.745 | 47.531 | 8.319 | 1.00 | 110.90 |
| 3437 | CA | ASN B | 50 | 37.830 | 47.919 | 9.213 | 1.00 | 110.90 |
| 3438 | CB | ASN B | 50 | 39.130 | 48.104 | 8.418 | 1.00 | 249.40 |
| 3439 | CG | ASN B | 50 | 40.355 | 48.183 | 9.311 | 1.00 | 249.40 |
| 3440 | OD1 | ASN B | 50 | 40.232 | 48.440 | 10.508 | 1.00 | 249.40 |
| 3441 | ND2 | ASN B | 50 | 41.539 | 47.979 | 8.738 | 1.00 | 249.40 |
| 3442 | C | ASN B | 50 | 37.403 | 49.246 | 9.854 | 1.00 | 110.90 |
| 3443 | O | ASN B | 50 | 36.644 | 50.010 | 9.250 | 1.00 | 110.90 |
| 3444 | N | SER B | 51 | 37.872 | 49.520 | 11.072 | 1.00 | 116.16 |
| 3445 | CA | SER B | 51 | 37.515 | 50.763 | 11.761 | 1.00 | 116.16 |
| 3446 | CB | SER B | 51 | 38.004 | 50.728 | 13.210 | 1.00 | 152.88 |
| 3447 | OG | SER B | 51 | 39.421 | 50.782 | 13.277 | 1.00 | 152.88 |
| 3448 | C | SER B | 51 | 38.084 | 52.007 | 11.066 | 1.00 | 116.16 |
| 3449 | O | SER B | 51 | 37.632 | 53.121 | 11.313 | 1.00 | 116.16 |
| 3450 | N | SER B | 52 | 39.080 | 51.819 | 10.206 | 1.00 | 154.44 |
| 3451 | CA | SER B | 52 | 39.684 | 52.939 | 9.501 | 1.00 | 154.44 |
| 3452 | CB | SER B | 52 | 41.210 | 52.899 | 9.637 | 1.00 | 81.31 |
| 3453 | OG | SER B | 52 | 41.611 | 53.078 | 10.987 | 1.00 | 81.31 |
| 3454 | C | SER B | 52 | 39.294 | 52.908 | 8.036 | 1.00 | 154.44 |
| 3455 | O | SER B | 52 | 39.754 | 52.057 | 7.273 | 1.00 | 154.44 |
| 3456 | N | LEU B | 53 | 38.433 | 53.844 | 7.656 | 1.00 | 115.15 |
| 3457 | CA | LEU B | 53 | 37.961 | 53.963 | 6.280 | 1.00 | 115.15 |
| 3458 | CB | LEU B | 53 | 36.477 | 54.348 | 6.281 | 1.00 | 65.06 |
| 3459 | CG | LEU B | 53 | 35.882 | 55.021 | 5.036 | 1.00 | 65.06 |
| 3460 | CD1 | LEU B | 53 | 36.353 | 54.301 | 3.774 | 1.00 | 65.06 |
| 3461 | CD2 | LEU B | 53 | 34.357 | 55.039 | 5.132 | 1.00 | 65.06 |
| 3462 | C | LEU B | 53 | 38.775 | 55.005 | 5.509 | 1.00 | 115.15 |
| 3463 | O | LEU B | 53 | 38.547 | 56.209 | 5.659 | 1.00 | 115.15 |
| 3464 | N | ASN B | 54 | 39.712 | 54.549 | 4.676 | 1.00 | 78.33 |
| 3465 | CA | ASN B | 54 | 40.533 | 55.488 | 3.918 | 1.00 | 78.33 |
| 3466 | CB | ASN B | 54 | 41.826 | 54.832 | 3.460 | 1.00 | 116.91 |
| 3467 | CG | ASN B | 54 | 42.792 | 54.609 | 4.598 | 1.00 | 116.91 |
| 3468 | OD1 | ASN B | 54 | 43.166 | 55.545 | 5.307 | 1.00 | 116.91 |
| 3469 | ND2 | ASN B | 54 | 43.204 | 53.364 | 4.782 | 1.00 | 116.91 |
| 3470 | C | ASN B | 54 | 39.834 | 56.084 | 2.716 | 1.00 | 78.33 |
| 3471 | O | ASN B | 54 | 38.853 | 55.548 | 2.226 | 1.00 | 78.33 |
| 3472 | N | ILE B | 55 | 40.333 | 57.227 | 2.269 | 1.00 | 83.98 |
| 3473 | CA | ILE B | 55 | 39.800 | 57.906 | 1.100 | 1.00 | 83.98 |
| 3474 | CB | ILE B | 55 | 38.973 | 59.141 | 1.493 | 1.00 | 67.63 |
| 3475 | CG2 | ILE B | 55 | 38.828 | 60.092 | 0.322 | 1.00 | 67.63 |
| 3476 | CG1 | ILE B | 55 | 37.598 | 58.686 | 1.980 | 1.00 | 67.63 |
| 3477 | CD1 | ILE B | 55 | 36.675 | 59.826 | 2.438 | 1.00 | 67.63 |
| 3473 | C | ILE B | 55 | 41.015 | 58.329 | 0.298 | 1.00 | 83.98 |
| 3479 | O | ILE B | 55 | 41.882 | 59.043 | 0.805 | 1.00 | 83.98 |
| 3480 | N | VAL B | 56 | 41.099 | 57.866 | 0.942 | 1.00 | 110.45 |
| 3481 | CA | VAL B | 56 | 42.231 | 58.216 | −1.781 | 1.00 | 110.45 |
| 3482 | CB | VAL B | 56 | 42.737 | 56.993 | −2.541 | 1.00 | 102.53 |
| 3483 | CG1 | VAL B | 56 | 44.131 | 57.246 | −3.058 | 1.00 | 102.53 |
| 3484 | CG2 | VAL B | 56 | 42.749 | 55.791 | −1.618 | 1.00 | 102.53 |
| 3485 | C | VAL B | 56 | 41.796 | 59.306 | −2.748 | 1.00 | 110.45 |
| 3486 | O | VAL B | 56 | 40.783 | 59.952 | −2.515 | 1.00 | 110.45 |
| 3487 | N | ASN B | 57 | 42.556 | 59.515 | −3.820 | 1.00 | 137.41 |
| 3488 | CA | ASN B | 57 | 42.235 | 60.554 | −4.794 | 1.00 | 137.41 |
| 3489 | CB | ASN B | 57 | 42.508 | 60.063 | −6.216 | 1.00 | 211.36 |
| 3490 | CG | ASN B | 57 | 43.990 | 59.900 | −6.493 | 1.00 | 211.36 |
| 3491 | OD1 | ASN B | 57 | 44.776 | 60.825 | −6.294 | 1.00 | 211.36 |
| 3492 | ND2 | ASN B | 57 | 44.379 | 58.721 | −6.959 | 1.00 | 211.36 |
| 3493 | C | ASN B | 57 | 40.795 | 61.032 | −4.667 | 1.00 | 137.41 |
| 3494 | O | ASN B | 57 | 39.885 | 60.501 | −5.305 | 1.00 | 137.41 |
| 3495 | N | ALA B | 58 | 40.608 | 62.038 | −3.818 | 1.00 | 74.20 |
| 3496 | CA | ALA B | 58 | 39.303 | 62.603 | −3.561 | 1.00 | 74.20 |
| 3497 | CB | ALA B | 58 | 39.440 | 63.783 | −2.609 | 1.00 | 169.14 |
| 3498 | C | ALA B | 58 | 38.534 | 63.034 | −4.817 | 1.00 | 74.20 |
| 3499 | O | ALA B | 58 | 38.983 | 63.895 | −5.579 | 1.00 | 74.20 |
| 3500 | N | LYS B | 59 | 37.366 | 62.433 | −5.015 | 1.00 | 107.95 |
| 3501 | CA | LYS B | 59 | 36.507 | 62.766 | −6.137 | 1.00 | 107.95 |
| 3502 | CB | LYS B | 59 | 36.037 | 61.485 | −6.837 | 1.00 | 214.35 |
| 3503 | CG | LYS B | 59 | 37.184 | 60.618 | −7.354 | 1.00 | 214.35 |
| 3504 | CD | LYS B | 59 | 36.703 | 59.292 | −7.930 | 1.00 | 214.35 |
| 3505 | CE | LYS B | 59 | 37.872 | 58.451 | −8.432 | 1.00 | 214.35 |
| 3506 | NZ | LYS B | 59 | 37.428 | 57.138 | −8.972 | 1.00 | 214.35 |
| 3507 | C | LYS B | 59 | 35.330 | 63.514 | −5.521 | 1.00 | 107.95 |
| 3508 | O | LYS B | 59 | 34.924 | 63.205 | −4.397 | 1.00 | 107.95 |
| 3509 | N | PHE B | 60 | 34.798 | 64.502 | −6.234 | 1.00 | 88.51 |
| 3510 | CA | PHE B | 60 | 33.670 | 65.268 | −5.716 | 1.00 | 88.51 |
| 3511 | CB | PHE B | 60 | 33.032 | 66.051 | −6.845 | 1.00 | 104.07 |
| 3512 | CG | PHE B | 60 | 33.926 | 67.085 | −7.419 | 1.00 | 104.07 |
| 3513 | CD1 | PHE B | 60 | 33.803 | 67.477 | −8.736 | 1.00 | 104.07 |
| 3514 | CD2 | PHE B | 60 | 34.893 | 67.682 | −6.635 | 1.00 | 104.07 |
| 3515 | CE1 | PHE B | 60 | 34.629 | 68.451 | −9.266 | 1.00 | 104.07 |
| 3516 | CE2 | PHE B | 60 | 35.725 | 68.654 | −7.155 | 1.00 | 104.07 |
| 3517 | CZ | PHE B | 60 | 35.592 | 69.038 | −8.473 | 1.00 | 104.07 |
| 3518 | C | PHE B | 60 | 32.616 | 64.397 | −5.032 | 1.00 | 88.51 |
| 3519 | O | PHE B | 60 | 31.988 | 64.816 | −4.060 | 1.00 | 88.51 |
| 3520 | N | GLU B | 61 | 32.438 | 63.177 | −5.536 | 1.00 | 122.82 |
| 3521 | CA | GLU B | 61 | 31.453 | 62.251 | −4.988 | 1.00 | 122.82 |
| 3522 | CB | GLU B | 61 | 31.362 | 60.981 | −5.838 | 1.00 | 242.03 |
| 3523 | CG | GLU B | 61 | 30.921 | 61.202 | −7.268 | 1.00 | 242.03 |
| 3524 | CD | GLU B | 61 | 31.866 | 62.107 | −8.030 | 1.00 | 242.03 |
| 3525 | OE1 | GLU B | 61 | 33.080 | 61.812 | −8.056 | 1.00 | 242.03 |
| 3526 | OE2 | GLU B | 61 | 31.395 | 63.111 | −8.604 | 1.00 | 242.03 |
| 3527 | C | GLU B | 61 | 31.772 | 61.856 | −3.563 | 1.00 | 122.82 |
| 3528 | O | GLU B | 61 | 30.884 | 61.426 | −2.829 | 1.00 | 122.82 |
| 3529 | N | ASP B | 62 | 33.038 | 61.982 | −3.173 | 1.00 | 75.67 |
| 3530 | CA | ASP B | 62 | 33.435 | 61.622 | −1.821 | 1.00 | 75.67 |
| 3531 | CB | ASP B | 62 | 34.954 | 61.524 | −1.708 | 1.00 | 186.17 |
| 3532 | CG | ASP B | 62 | 35.544 | 60.572 | −2.723 | 1.00 | 186.17 |
| 3533 | OD1 | ASP B | 62 | 34.918 | 59.528 | −2.996 | 1.00 | 186.17 |
| 3534 | OD2 | ASP B | 62 | 36.638 | 60.860 | −3.242 | 1.00 | 186.17 |
| 3535 | C | ASP B | 62 | 32.889 | 62.643 | −0.834 | 1.00 | 75.67 |
| 3536 | O | ASP B | 62 | 32.765 | 62.359 | 0.354 | 1.00 | 75.67 |
| 3537 | N | SER B | 63 | 32.553 | 63.832 | −1.330 | 1.00 | 56.90 |
| 3538 | CA | SER B | 63 | 31.993 | 64.872 | −0.471 | 1.00 | 56.90 |
| 3539 | CB | SER B | 63 | 31.659 | 66.117 | −1.286 | 1.00 | 80.16 |
| 3540 | OG | SER B | 63 | 32.823 | 66.739 | −1.783 | 1.00 | 80.16 |
| 3541 | C | SER B | 63 | 30.710 | 64.291 | 0.102 | 1.00 | 56.90 |
| 3542 | O | SER B | 63 | 29.919 | 63.744 | −0.643 | 1.00 | 56.90 |
| 3543 | N | GLY B | 64 | 30.482 | 64.385 | 1.407 | 1.00 | 91.31 |
| 3544 | CA | GLY B | 64 | 29.254 | 63.819 | 1.941 | 1.00 | 91.31 |
| 3545 | C | GLY B | 64 | 29.177 | 63.712 | 3.447 | 1.00 | 91.31 |
| 3546 | O | GLY B | 64 | 30.012 | 64.259 | 4.164 | 1.00 | 91.31 |
| 3547 | N | GLU B | 65 | 28.154 | 63.014 | 3.922 | 1.00 | 66.19 |
| 3548 | CA | GLU B | 65 | 27.919 | 62.813 | 5.351 | 1.00 | 66.19 |
| 3549 | CB | GLU B | 65 | 26.443 | 63.045 | 5.642 | 1.00 | 122.59 |
| 3550 | CG | GLU B | 65 | 25.981 | 62.639 | 7.018 | 1.00 | 122.59 |
| 3551 | CD | GLU B | 65 | 24.468 | 62.585 | 7.107 | 1.00 | 122.59 |
| 3552 | OE1 | GLU B | 65 | 23.856 | 61.738 | 6.417 | 1.00 | 122.59 |
| 3553 | OE2 | GLU B | 65 | 23.890 | 63.390 | 7.863 | 1.00 | 122.59 |
| 3554 | C | GLU B | 65 | 28.311 | 61.374 | 5.711 | 1.00 | 66.19 |
| 3555 | O | GLU B | 65 | 27.826 | 60.434 | 5.088 | 1.00 | 66.19 |
| 3556 | N | TYR B | 66 | 29.183 | 61.182 | 6.697 | 1.00 | 58.72 |
| 3557 | CA | TYR B | 66 | 29.603 | 59.833 | 7.060 | 1.00 | 58.72 |
| 3558 | CB | TYR B | 66 | 31.093 | 59.680 | 6.855 | 1.00 | 55.88 |
| 3559 | CG | TYR B | 66 | 31.576 | 59.790 | 5.452 | 1.00 | 55.88 |
| 3560 | CD1 | TYR B | 66 | 31.703 | 61.020 | 4.825 | 1.00 | 55.83 |
| 3561 | CE1 | TYR B | 66 | 32.243 | 61.109 | 3.548 | 1.00 | 55.88 |
| 3562 | CD2 | TYR B | 66 | 31.986 | 58.657 | 4.772 | 1.00 | 55.88 |
| 3563 | CE2 | TYR B | 66 | 32.521 | 58.726 | 3.505 | 1.00 | 55.88 |
| 3564 | CZ | TYR B | 66 | 32.655 | 59.947 | 2.896 | 1.00 | 55.88 |
| 3565 | OH | TYR B | 66 | 33.230 | 59.979 | 1.643 | 1.00 | 55.88 |
| 3566 | C | TYR B | 66 | 29.320 | 59.544 | 8.522 | 1.00 | 58.72 |
| 3567 | O | TYR B | 66 | 29.111 | 60.482 | 9.311 | 1.00 | 58.72 |
| 3568 | N | LYS B | 67 | 29.347 | 58.257 | 8.884 | 1.00 | 128.26 |
| 3569 | CA | LYS B | 67 | 29.129 | 57.823 | 10.269 | 1.00 | 128.26 |
| 3570 | CB | LYS B | 67 | 27.689 | 58.067 | 10.679 | 1.00 | 129.32 |
| 3571 | CG | LYS B | 67 | 26.702 | 57.535 | 9.689 | 1.00 | 129.32 |
| 3572 | CD | LYS B | 67 | 25.301 | 57.905 | 10.098 | 1.00 | 129.32 |
| 3573 | CE | LYS B | 67 | 24.314 | 57.545 | 9.007 | 1.00 | 129.32 |
| 3574 | N2 | LYS B | 67 | 22.936 | 57.963 | 9.372 | 1.00 | 129.32 |
| 3575 | C | LYS B | 67 | 29.460 | 56.351 | 10.466 | 1.00 | 128.26 |
| 3576 | O | LYS B | 67 | 29.434 | 55.570 | 9.516 | 1.00 | 128.26 |
| 3577 | N | CYS B | 68 | 29.793 | 55.971 | 11.696 | 1.00 | 93.88 |
| 3578 | CA | CYS B | 68 | 30.107 | 54.581 | 11.964 | 1.00 | 93.88 |
| 3579 | C | CYS B | 68 | 29.262 | 54.113 | 13.122 | 1.00 | 93.88 |
| 3580 | O | CYS B | 68 | 28.693 | 54.923 | 13.850 | 1.00 | 93.88 |
| 3581 | CB | CYS B | 68 | 31.609 | 54.381 | 12.247 | 1.00 | 200.62 |
| 3582 | SG | CYS B | 68 | 32.359 | 55.270 | 13.646 | 1.00 | 200.62 |
| 3583 | N | GLN B | 69 | 29.148 | 52.797 | 13.255 | 1.00 | 198.52 |
| 3584 | CA | GLN B | 69 | 28.375 | 52.172 | 14.318 | 1.00 | 198.52 |
| 3585 | CB | GLN B | 69 | 26.897 | 52.107 | 13.926 | 1.00 | 207.53 |
| 3586 | CG | GLN B | 69 | 26.082 | 51.114 | 14.734 | 1.00 | 207.53 |
| 3587 | CD | GLN B | 69 | 24.654 | 50.983 | 14.233 | 1.00 | 207.53 |
| 3588 | OE1 | GLN B | 69 | 24.419 | 50.759 | 13.044 | 1.00 | 207.53 |
| 3589 | NE2 | GLN B | 69 | 23.692 | 51.117 | 15.143 | 1.00 | 207.53 |
| 3590 | C | GLN B | 69 | 28.921 | 50.768 | 14.527 | 1.00 | 198.52 |
| 3591 | O | GLN B | 69 | 29.474 | 50.173 | 13.600 | 1.00 | 198.52 |
| 3592 | N | HIS B | 70 | 28.772 | 50.240 | 15.739 | 1.00 | 126.14 |
| 3593 | CA | HIS B | 70 | 29.266 | 48.903 | 16.020 | 1.00 | 126.14 |
| 3594 | CB | HIS B | 70 | 30.134 | 48.915 | 17.265 | 1.00 | 193.31 |
| 3595 | CG | HIS B | 70 | 31.435 | 49.650 | 17.083 | 1.00 | 193.31 |
| 3596 | CD2 | HIS B | 70 | 31.809 | 50.892 | 17.450 | 1.00 | 193.31 |
| 3597 | ND1 | HIS B | 70 | 32.502 | 49.089 | 16.411 | 1.00 | 193.31 |
| 3598 | CE1 | HIS B | 70 | 33.480 | 49.982 | 16.371 | 1.00 | 193.31 |
| 3599 | NE2 | HIS B | 70 | 33.095 | 51.074 | 16.992 | 1.00 | 193.31 |
| 3600 | C | HIS B | 70 | 28.144 | 47.890 | 16.193 | 1.00 | 126.14 |
| 3601 | O | HIS B | 70 | 26.974 | 48.180 | 15.915 | 1.00 | 126.14 |
| 3602 | N | GLN B | 71 | 28.511 | 46.697 | 16.639 | 1.00 | 181.78 |
| 3603 | CA | GLN B | 71 | 27.558 | 45.621 | 16.836 | 1.00 | 181.78 |
| 3604 | CB | GLN B | 71 | 28.277 | 44.424 | 17.456 | 1.00 | 249.38 |
| 3605 | CG | GLN B | 71 | 27.687 | 43.082 | 17.057 | 1.00 | 249.38 |
| 3606 | CD | GLN B | 71 | 27.525 | 42.946 | 15.553 | 1.00 | 249.38 |
| 3607 | OE1 | GLN B | 71 | 28.501 | 42.814 | 14.816 | 1.00 | 249.38 |
| 3608 | NE2 | GLN B | 71 | 26.283 | 42.994 | 15.091 | 1.00 | 249.38 |
| 3609 | C | GLN B | 71 | 26.374 | 46.062 | 17.711 | 1.00 | 181.78 |
| 3610 | O | GLN B | 71 | 25.214 | 45.948 | 17.300 | 1.00 | 181.78 |
| 3611 | N | GLN B | 72 | 26.666 | 46.582 | 18.902 | 1.00 | 249.48 |
| 3612 | CA | GLN B | 72 | 25.627 | 47.029 | 19.838 | 1.00 | 249.48 |
| 3613 | CB | GLN B | 72 | 25.631 | 46.132 | 21.084 | 1.00 | 225.39 |
| 3614 | CG | GLN B | 72 | 24.511 | 46.421 | 22.083 | 1.00 | 225.39 |
| 3615 | CD | GLN B | 72 | 24.526 | 45.478 | 23.273 | 1.00 | 225.39 |
| 3616 | OE1 | GLN B | 72 | 24.436 | 44.260 | 23.115 | 1.00 | 225.39 |
| 3617 | NE2 | GLN B | 72 | 24.641 | 46.038 | 24.471 | 1.00 | 225.39 |
| 3618 | C | GLN B | 72 | 25.812 | 48.487 | 20.262 | 1.00 | 249.48 |
| 3619 | O | GLN B | 72 | 25.935 | 48.787 | 21.455 | 1.00 | 249.48 |
| 3620 | N | VAL B | 73 | 25.821 | 49.395 | 19.288 | 1.00 | 181.22 |
| 3621 | CA | VAL B | 73 | 26.005 | 50.817 | 19.583 | 1.00 | 181.22 |
| 3622 | CB | VAL B | 73 | 27.465 | 51.205 | 19.481 | 1.00 | 249.28 |
| 3623 | CG1 | VAL B | 73 | 27.738 | 52.552 | 20.109 | 1.00 | 249.28 |
| 3624 | CG2 | VAL B | 73 | 28.193 | 50.235 | 20.151 | 1.00 | 249.28 |
| 3625 | C | VAL B | 73 | 25.240 | 51.690 | 18.626 | 1.00 | 181.22 |
| 3626 | O | VAL B | 73 | 25.071 | 51.348 | 17.462 | 1.00 | 181.22 |
| 3627 | N | ASN B | 74 | 24.776 | 52.826 | 19.122 | 1.00 | 246.14 |
| 3628 | CA | ASN B | 74 | 24.042 | 53.744 | 18.278 | 1.00 | 246.14 |
| 3629 | CB | ASN B | 74 | 23.201 | 54.681 | 19.141 | 1.00 | 197.13 |
| 3630 | CG | ASN B | 74 | 22.296 | 53.925 | 20.082 | 1.00 | 197.13 |
| 3631 | OD1 | ASN B | 74 | 21.682 | 52.931 | 19.688 | 1.00 | 197.13 |
| 3632 | ND2 | ASN B | 74 | 22.202 | 54.394 | 21.324 | 1.00 | 197.13 |
| 3633 | C | ASN B | 74 | 25.027 | 54.526 | 17.418 | 1.00 | 246.14 |
| 3634 | O | ASN B | 74 | 26.004 | 55.081 | 17.922 | 1.00 | 246.14 |
| 3635 | N | GLU B | 75 | 24.761 | 54.542 | 16.114 | 1.00 | 146.48 |
| 3636 | CA | GLU B | 75 | 25.597 | 55.232 | 15.135 | 1.00 | 146.48 |
| 3637 | CB | GLU B | 75 | 24.848 | 55.331 | 13.807 | 1.00 | 234.88 |
| 3638 | CG | GLU B | 75 | 23.346 | 55.500 | 13.966 | 1.00 | 234.88 |
| 3639 | CD | GLU B | 75 | 22.604 | 55.365 | 12.648 | 1.00 | 234.88 |
| 3640 | OE1 | GLU B | 75 | 22.784 | 54.328 | 11.970 | 1.00 | 234.88 |
| 3641 | OE2 | GLU B | 75 | 21.840 | 56.291 | 12.295 | 1.00 | 234.88 |
| 3642 | C | GLU B | 75 | 26.075 | 56.613 | 15.579 | 1.00 | 146.48 |
| 3643 | O | GLU B | 75 | 25.344 | 57.361 | 16.239 | 1.00 | 146.48 |
| 3644 | N | SER B | 76 | 27.311 | 56.939 | 15.201 | 1.00 | 102.01 |
| 3645 | CA | SER B | 76 | 27.958 | 58.200 | 15.564 | 1.00 | 102.01 |
| 3646 | CB | SER B | 76 | 29.420 | 58.153 | 15.147 | 1.00 | 220.64 |
| 3647 | OG | SER B | 76 | 29.501 | 58.098 | 13.732 | 1.00 | 220.64 |
| 3648 | C | SER B | 76 | 27.336 | 59.426 | 14.930 | 1.00 | 102.01 |
| 3649 | O | SER B | 76 | 26.652 | 59.332 | 13.921 | 1.00 | 102.01 |
| 3650 | N | GLU B | 77 | 27.604 | 60.583 | 15.522 | 1.00 | 133.62 |
| 3651 | CA | GLU B | 77 | 27.102 | 61.838 | 14.988 | 1.00 | 133.62 |
| 3652 | CB | GLU B | 77 | 27.429 | 62.993 | 15.941 | 1.00 | 240.61 |
| 3653 | CG | GLU B | 77 | 26.732 | 62.894 | 17.288 | 1.00 | 240.61 |
| 3654 | CD | GLU B | 77 | 25.218 | 62.995 | 17.179 | 1.00 | 240.61 |
| 3655 | OE1 | GLU B | 77 | 24.686 | 62.847 | 16.060 | 1.00 | 240.61 |
| 3656 | OE2 | GLU B | 77 | 24.558 | 63.213 | 18.217 | 1.00 | 240.61 |
| 3657 | C | GLU B | 77 | 27.798 | 62.057 | 13.651 | 1.00 | 133.62 |
| 3658 | O | GLU B | 77 | 29.023 | 62.177 | 13.597 | 1.00 | 133.62 |
| 3659 | N | PRO B | 78 | 27.030 | 62.096 | 12.549 | 1.00 | 89.52 |
| 3660 | CD | PRO B | 78 | 25.577 | 61.836 | 12.507 | 1.00 | 87.18 |
| 3661 | CA | PRO B | 78 | 27.543 | 62.294 | 11.196 | 1.00 | 89.52 |
| 3662 | CB | PRO B | 78 | 26.295 | 62.633 | 10.408 | 1.00 | 87.18 |
| 3663 | CG | PRO B | 78 | 25.313 | 61.698 | 11.009 | 1.00 | 87.18 |
| 3664 | C | PRO B | 78 | 28.586 | 63.370 | 11.098 | 1.00 | 89.52 |
| 3665 | O | PRO B | 78 | 28.644 | 64.258 | 11.943 | 1.00 | 89.52 |
| 3666 | N | VAL B | 79 | 29.430 | 63.270 | 10.082 | 1.00 | 99.13 |
| 3667 | CA | VAL B | 79 | 30.475 | 64.256 | 9.872 | 1.00 | 99.13 |
| 3668 | CB | VAL B | 79 | 31.833 | 63.720 | 10.291 | 1.00 | 115.84 |
| 3669 | CG1 | VAL B | 79 | 32.929 | 64.618 | 9.749 | 1.00 | 115.84 |
| 3670 | CG2 | VAL B | 79 | 31.908 | 63.671 | 11.798 | 1.00 | 115.84 |
| 3671 | C | VAL B | 79 | 30.512 | 64.571 | 8.401 | 1.00 | 99.13 |
| 3672 | O | VAL B | 79 | 30.573 | 63.654 | 7.592 | 1.00 | 99.13 |
| 3673 | N | TYR B | 80 | 30.480 | 65.853 | 8.044 | 1.00 | 70.58 |
| 3674 | CA | TYR B | 80 | 30.490 | 66.213 | 6.637 | 1.00 | 70.58 |
| 3675 | CB | TYR B | 80 | 29.622 | 67.426 | 6.352 | 1.00 | 173.93 |
| 3676 | CG | TYR B | 80 | 29.319 | 67.524 | 4.884 | 1.00 | 173.93 |
| 3677 | CD1 | TYR B | 80 | 28.546 | 66.550 | 4.261 | 1.00 | 173.93 |
| 3678 | CE1 | TYR B | 80 | 28.266 | 66.604 | 2.913 | 1.00 | 173.93 |
| 3679 | CD2 | TYR B | 80 | 29.824 | 68.559 | 4.106 | 1.00 | 173.93 |
| 3680 | CE2 | TYR B | 80 | 29.561 | 68.613 | 2.729 | 1.00 | 173.93 |
| 3681 | CZ | TYR B | 80 | 28.769 | 67.630 | 2.148 | 1.00 | 173.93 |
| 3682 | OH | TYR B | 80 | 28.432 | 67.702 | 0.811 | 1.00 | 173.93 |
| 3683 | C | TYR B | 80 | 31.858 | 66.511 | 6.103 | 1.00 | 70.58 |
| 3684 | O | TYR B | 80 | 32.657 | 67.183 | 6.739 | 1.00 | 70.58 |
| 3685 | N | LEU B | 81 | 32.109 | 66.033 | 4.902 | 1.00 | 86.81 |
| 3686 | CA | LEU B | 81 | 33.375 | 66.256 | 4.254 | 1.00 | 86.81 |
| 3687 | CB | LEU B | 81 | 34.030 | 64.899 | 3.970 | 1.00 | 52.23 |
| 3688 | CG | LEU B | 81 | 35.301 | 65.038 | 3.148 | 1.00 | 52.23 |
| 3689 | CD1 | LEU B | 81 | 36.303 | 65.810 | 3.970 | 1.00 | 52.23 |
| 3690 | CD2 | LEU B | 81 | 35.843 | 63.724 | 2.783 | 1.00 | 52.23 |
| 3691 | C | LEU B | 81 | 33.090 | 66.999 | 2.944 | 1.00 | 86.81 |
| 3692 | O | LEU B | 81 | 32.240 | 66.576 | 2.171 | 1.00 | 86.81 |
| 3693 | N | GLU B | 82 | 33.777 | 68.104 | 2.682 | 1.00 | 81.52 |
| 3694 | CA | GLU B | 82 | 33.537 | 68.808 | 1.430 | 1.00 | 81.52 |
| 3695 | CB | GLU B | 82 | 33.000 | 70.212 | 1.700 | 1.00 | 166.15 |
| 3696 | CG | GLU B | 82 | 32.168 | 70.757 | 0.552 | 1.00 | 166.15 |
| 3697 | CD | GLU B | 82 | 31.619 | 72.139 | 0.827 | 1.00 | 166.15 |
| 3698 | OE1 | GLU B | 82 | 31.239 | 72.409 | 1.988 | 1.00 | 166.15 |
| 3699 | OE2 | GLU B | 82 | 31.557 | 72.953 | −0.118 | 1.00 | 166.15 |
| 3700 | C | GLU B | 82 | 34.800 | 68.898 | 0.584 | 1.00 | 81.52 |
| 3701 | O | GLU B | 82 | 35.856 | 69.283 | 1.072 | 1.00 | 81.52 |
| 3702 | N | VAL B | 83 | 34.691 | 68.555 | −0.692 | 1.00 | 81.14 |
| 3703 | CA | VAL B | 83 | 35.842 | 68.595 | −1.584 | 1.00 | 81.14 |
| 3704 | CB | VAL B | 83 | 35.910 | 67.346 | −2.417 | 1.00 | 54.13 |
| 3705 | CG1 | VAL B | 83 | 37.014 | 67.472 | −3.433 | 1.00 | 54.13 |
| 3706 | CG2 | VAL B | 83 | 36.159 | 66.136 | −1.512 | 1.00 | 54.13 |
| 3707 | C | VAL B | 83 | 35.848 | 69.781 | −2.535 | 1.00 | 81.14 |
| 3708 | O | VAL B | 83 | 34.831 | 70.075 | −3.168 | 1.00 | 81.14 |
| 3709 | N | PHE B | 84 | 37.000 | 70.441 | −2.667 | 1.00 | 61.79 |
| 3710 | CA | PHE B | 84 | 37.084 | 71.612 | −3.530 | 1.00 | 61.79 |
| 3711 | CB | PHE B | 84 | 37.407 | 72.864 | −2.729 | 1.00 | 77.82 |
| 3712 | CG | PHE B | 84 | 36.432 | 73.162 | −1.660 | 1.00 | 77.82 |
| 3713 | CD1 | PHE B | 84 | 36.408 | 72.410 | −0.500 | 1.00 | 77.82 |
| 3714 | CD2 | PHE B | 84 | 35.555 | 74.223 | −1.790 | 1.00 | 77.82 |
| 3715 | CE1 | PHE B | 84 | 35.513 | 72.699 | 0.522 | 1.00 | 77.82 |
| 3716 | CE2 | PHE B | 84 | 34.650 | 74.529 | −0.779 | 1.00 | 77.82 |
| 3717 | CZ | PHE B | 84 | 34.634 | 73.766 | 0.384 | 1.00 | 77.82 |
| 3718 | C | PHE B | 84 | 38.081 | 71.568 | −4.654 | 1.00 | 61.79 |
| 3719 | O | PHE B | 84 | 38.978 | 70.728 | −4.701 | 1.00 | 61.79 |
| 3720 | N | SER B | 85 | 37.893 | 72.538 | −5.543 | 1.00 | 129.28 |
| 3721 | CA | SER B | 85 | 38.736 | 72.777 | −6.696 | 1.00 | 129.28 |
| 3722 | CB | SER B | 85 | 38.066 | 72.284 | −7.980 | 1.00 | 132.41 |
| 3723 | OG | SER B | 85 | 38.879 | 72.526 | −9.116 | 1.00 | 132.41 |
| 3724 | C | SER B | 85 | 38.840 | 74.296 | −6.713 | 1.00 | 129.28 |
| 3725 | O | SER B | 85 | 37.845 | 74.988 | −6.967 | 1.00 | 129.28 |
| 3726 | N | ASP B | 86 | 40.026 | 74.811 | −6.395 | 1.00 | 77.53 |
| 3727 | CA | ASP B | 86 | 40.255 | 76.257 | −6.385 | 1.00 | 77.53 |
| 3728 | CB | ASP B | 86 | 39.348 | 76.933 | −5.354 | 1.00 | 206.86 |
| 3729 | CG | ASP B | 86 | 38.874 | 78.300 | −5.809 | 1.00 | 206.86 |
| 3730 | OD1 | ASP B | 86 | 39.733 | 79.139 | −6.163 | 1.00 | 206.86 |
| 3731 | OD2 | ASP B | 86 | 37.644 | 78.535 | −5.812 | 1.00 | 206.86 |
| 3732 | C | ASP B | 86 | 41.719 | 76.537 | −6.065 | 1.00 | 77.53 |
| 3733 | O | ASP B | 86 | 42.423 | 75.643 | −5.601 | 1.00 | 77.53 |
| 3734 | N | TRP B | 87 | 42.186 | 77.759 | −6.313 | 1.00 | 63.09 |
| 3735 | CA | TRP B | 87 | 43.589 | 78.072 | −6.048 | 1.00 | 63.09 |
| 3736 | CB | TRP B | 87 | 43.934 | 79.488 | −6.505 | 1.00 | 213.86 |
| 3737 | CG | TRP B | 87 | 44.332 | 79.502 | −7.919 | 1.00 | 213.86 |
| 3738 | CD2 | TRP B | 87 | 43.467 | 79.711 | −9.031 | 1.00 | 213.86 |
| 3739 | CE2 | TRP B | 87 | 44.225 | 79.492 | −10.196 | 1.00 | 213.86 |
| 3740 | CE3 | TRP B | 87 | 42.115 | 80.053 | −9.157 | 1.00 | 213.86 |
| 3741 | CD1 | TRP B | 87 | 45.561 | 79.186 | −8.435 | 1.00 | 213.86 |
| 3742 | NE1 | TRP B | 87 | 45.500 | 79.175 | −9.807 | 1.00 | 213.86 |
| 3743 | CZ2 | TRP B | 87 | 43.674 | 79.604 | −11.468 | 1.00 | 213.86 |
| 3744 | CZ3 | TRP B | 87 | 41.570 | 80.162 | −10.423 | 1.00 | 213.86 |
| 3745 | CH2 | TRP B | 87 | 42.347 | 79.943 | −11.559 | 1.00 | 213.86 |
| 3746 | C | TRP B | 87 | 43.913 | 77.935 | −4.589 | 1.00 | 63.09 |
| 3747 | O | TRP B | 87 | 44.856 | 77.221 | −4.208 | 1.00 | 63.09 |
| 3748 | N | LEU B | 88 | 43.110 | 78.622 | −3.783 | 1.00 | 95.94 |
| 3749 | CA | LEU B | 88 | 43.280 | 78.617 | −2.349 | 1.00 | 95.94 |
| 3750 | CB | LEU B | 88 | 43.600 | 80.021 | −1.861 | 1.00 | 93.07 |
| 3751 | CG | LEU B | 88 | 44.931 | 80.558 | −2.325 | 1.00 | 93.07 |
| 3752 | CD1 | LEU B | 88 | 45.167 | 81.882 | −1.668 | 1.00 | 93.07 |
| 3753 | CD2 | LEU B | 88 | 46.019 | 79.559 | −1.955 | 1.00 | 93.07 |
| 3754 | C | LEU B | 88 | 42.050 | 78.126 | −1.621 | 1.00 | 95.94 |
| 3755 | O | LEU B | 88 | 40.927 | 78.425 | −2.004 | 1.00 | 95.94 |
| 3756 | N | LEU B | 89 | 42.276 | 77.380 | −0.550 | 1.00 | 57.56 |
| 3757 | CA | LEU B | 89 | 41.191 | 76.863 | 0.265 | 1.00 | 57.56 |
| 3758 | CB | LEU B | 89 | 41.063 | 75.370 | 0.059 | 1.00 | 98.29 |
| 3759 | CG | LEU B | 89 | 39.972 | 74.802 | 0.940 | 1.00 | 98.29 |
| 3760 | CD1 | LEU B | 89 | 38.700 | 75.640 | 0.767 | 1.00 | 98.29 |
| 3761 | CD2 | LEU B | 89 | 39.741 | 73.357 | 0.564 | 1.00 | 98.29 |
| 3762 | C | LEU B | 89 | 41.488 | 77.138 | 1.724 | 1.00 | 57.56 |
| 3763 | O | LEU B | 89 | 42.566 | 76.832 | 2.192 | 1.00 | 57.56 |
| 3764 | N | LEU B | 90 | 40.553 | 77.737 | 2.444 | 1.00 | 82.03 |
| 3765 | CA | LEU B | 90 | 40.787 | 78.008 | 3.857 | 1.00 | 82.03 |
| 3766 | CB | LEU B | 90 | 40.005 | 79.244 | 4.303 | 1.00 | 51.54 |
| 3767 | CG | LEU B | 90 | 40.073 | 79.537 | 5.807 | 1.00 | 51.54 |
| 3768 | CD1 | LEU B | 90 | 41.486 | 79.805 | 6.154 | 1.00 | 51.54 |
| 3769 | CD2 | LEU B | 90 | 39.203 | 80.720 | 6.203 | 1.00 | 51.54 |
| 3770 | C | LEU B | 90 | 40.347 | 76.808 | 4.674 | 1.00 | 82.03 |
| 3771 | O | LEU B | 90 | 39.173 | 76.431 | 4.667 | 1.00 | 82.03 |
| 3772 | N | GLN B | 91 | 41.274 | 76.199 | 5.391 | 1.00 | 55.00 |
| 3773 | CA | GLN B | 91 | 40.904 | 75.024 | 6.182 | 1.00 | 55.00 |
| 3774 | CB | GLN B | 91 | 41.909 | 73.900 | 5.955 | 1.00 | 79.62 |
| 3775 | CG | GLN B | 91 | 42.017 | 73.500 | 4.501 | 1.00 | 79.62 |
| 3776 | CD | GLN B | 91 | 42.871 | 72.287 | 4.316 | 1.00 | 79.62 |
| 3777 | OE1 | GLN B | 91 | 44.072 | 72.334 | 4.524 | 1.00 | 79.62 |
| 3778 | NE2 | GLN B | 91 | 42.253 | 71.180 | 3.942 | 1.00 | 79.62 |
| 3779 | C | GLN B | 91 | 40.793 | 75.316 | 7.670 | 1.00 | 55.00 |
| 3780 | O | GLN B | 91 | 41.552 | 76.118 | 8.212 | 1.00 | 55.00 |
| 3781 | N | ALA B | 92 | 39.846 | 74.680 | 8.344 | 1.00 | 72.63 |
| 3782 | CA | ALA B | 92 | 39.692 | 74.939 | 9.760 | 1.00 | 72.63 |
| 3783 | CB | ALA B | 92 | 38.406 | 75.678 | 10.004 | 1.00 | 131.49 |
| 3784 | C | ALA B | 92 | 39.691 | 73.632 | 10.519 | 1.00 | 72.63 |
| 3785 | O | ALA B | 92 | 39.122 | 72.634 | 10.050 | 1.00 | 72.63 |
| 3786 | N | SER B | 93 | 40.338 | 73.624 | 11.685 | 1.00 | 73.84 |
| 3787 | CA | SER B | 93 | 40.381 | 72.421 | 12.512 | 1.00 | 73.84 |
| 3788 | CB | SER B | 93 | 41.018 | 72.709 | 13.873 | 1.00 | 152.84 |
| 3789 | OG | SER B | 93 | 40.445 | 73.845 | 14.491 | 1.00 | 152.84 |
| 3790 | C | SER B | 93 | 38.934 | 72.013 | 12.691 | 1.00 | 73.84 |
| 3791 | O | SER B | 93 | 38.515 | 70.973 | 12.179 | 1.00 | 73.84 |
| 3792 | N | ALA B | 94 | 38.167 | 72.859 | 13.378 | 1.00 | 105.05 |
| 3793 | CA | ALA B | 94 | 36.743 | 72.624 | 13.619 | 1.00 | 105.05 |
| 3794 | CB | ALA B | 94 | 36.517 | 72.246 | 15.061 | 1.00 | 185.57 |
| 3795 | C | ALA B | 94 | 35.978 | 73.898 | 13.280 | 1.00 | 105.05 |
| 3796 | O | ALA B | 94 | 36.478 | 74.988 | 13.524 | 1.00 | 105.05 |
| 3797 | N | GLU B | 95 | 34.776 | 73.763 | 12.724 | 1.00 | 101.72 |
| 3798 | CA | GLU B | 95 | 34.005 | 74.936 | 12.340 | 1.00 | 101.72 |
| 3799 | CB | GLU B | 95 | 33.081 | 74.601 | 11.175 | 1.00 | 160.65 |
| 3800 | CG | GLU B | 95 | 33.822 | 74.120 | 9.941 | 1.00 | 160.65 |
| 3801 | CD | GLU B | 95 | 32.955 | 74.120 | 8.692 | 1.00 | 160.65 |
| 3802 | OE1 | GLU B | 95 | 33.455 | 73.705 | 7.625 | 1.00 | 160.65 |
| 3803 | OE2 | GLU B | 95 | 31.779 | 74.538 | 8.771 | 1.00 | 160.65 |
| 3804 | C | GLU B | 95 | 33.205 | 75.550 | 13.473 | 1.00 | 101.72 |
| 3805 | O | GLU B | 95 | 32.732 | 76.677 | 13.354 | 1.00 | 101.72 |
| 3806 | N | VAL B | 96 | 33.050 | 74.807 | 14.565 | 1.00 | 87.11 |
| 3807 | CA | VAL B | 96 | 32.322 | 75.296 | 15.730 | 1.00 | 87.11 |
| 3808 | CB | VAL B | 96 | 30.947 | 74.746 | 15.781 | 1.00 | 166.75 |
| 3809 | CG1 | VAL B | 96 | 30.147 | 75.595 | 16.714 | 1.00 | 166.75 |
| 3810 | CG2 | VAL B | 96 | 30.349 | 74.728 | 14.367 | 1.00 | 166.75 |
| 3811 | C | VAL B | 96 | 33.096 | 74.866 | 16.955 | 1.00 | 87.11 |
| 3812 | O | VAL B | 96 | 33.528 | 73.724 | 17.052 | 1.00 | 87.11 |
| 3813 | N | VAL B | 97 | 33.260 | 75.781 | 17.900 | 1.00 | 103.60 |
| 3814 | CA | VAL B | 97 | 34.080 | 75.505 | 19.067 | 1.00 | 103.60 |
| 3815 | CB | VAL B | 97 | 35.444 | 76.140 | 18.858 | 1.00 | 67.08 |
| 3816 | CG1 | VAL B | 97 | 36.415 | 75.622 | 19.857 | 1.00 | 67.08 |
| 3817 | CG2 | VAL B | 97 | 35.924 | 75.882 | 17.456 | 1.00 | 67.08 |
| 3818 | C | VAL B | 97 | 33.591 | 76.003 | 20.417 | 1.00 | 103.60 |
| 3819 | O | VAL B | 97 | 33.142 | 77.136 | 20.533 | 1.00 | 103.60 |
| 3820 | N | MET B | 98 | 33.730 | 75.168 | 21.441 | 1.00 | 173.13 |
| 3821 | CA | MET B | 98 | 33.341 | 75.542 | 22.798 | 1.00 | 173.13 |
| 3822 | CB | MET B | 98 | 33.361 | 74.306 | 23.696 | 1.00 | 240.86 |
| 3823 | CG | MET B | 98 | 32.369 | 73.237 | 23.290 | 1.00 | 240.86 |
| 3824 | SD | MET B | 98 | 30.722 | 73.639 | 23.866 | 1.00 | 240.86 |
| 3825 | CE | MET B | 98 | 30.921 | 73.267 | 25.612 | 1.00 | 240.86 |
| 3826 | C | MET B | 98 | 34.341 | 76.573 | 23.323 | 1.00 | 173.13 |
| 3827 | O | MET B | 98 | 35.547 | 76.380 | 23.185 | 1.00 | 173.13 |
| 3828 | N | GLU B | 99 | 33.849 | 77.658 | 23.918 | 1.00 | 116.59 |
| 3829 | CA | GLU B | 99 | 34.731 | 78.695 | 24.451 | 1.00 | 116.59 |
| 3830 | CB | GLU B | 99 | 33.954 | 79.631 | 25.376 | 1.00 | 249.41 |
| 3831 | CG | GLU B | 99 | 34.610 | 80.985 | 25.567 | 1.00 | 249.41 |
| 3832 | CD | GLU B | 99 | 34.016 | 81.763 | 26.727 | 1.00 | 249.41 |
| 3833 | OE1 | GLU B | 99 | 32.782 | 81.690 | 26.920 | 1.00 | 249.41 |
| 3834 | OE2 | GLU B | 99 | 34.781 | 82.455 | 27.435 | 1.00 | 249.41 |
| 3835 | C | GLU B | 99 | 35.853 | 78.023 | 25.247 | 1.00 | 116.59 |
| 3836 | O | GLU B | 99 | 35.582 | 77.203 | 26.127 | 1.00 | 116.59 |
| 3837 | N | GLY B | 100 | 37.106 | 78.351 | 24.935 | 1.00 | 84.75 |
| 3838 | CA | GLY B | 100 | 38.221 | 77.751 | 25.651 | 1.00 | 84.75 |
| 3839 | C | GLY B | 100 | 39.031 | 76.722 | 24.883 | 1.00 | 84.75 |
| 3840 | O | GLY B | 100 | 40.171 | 76.451 | 25.243 | 1.00 | 84.75 |
| 3841 | N | GLN B | 101 | 38.464 | 76.151 | 23.824 | 1.00 | 108.09 |
| 3842 | CA | GLN B | 101 | 39.167 | 75.134 | 23.033 | 1.00 | 108.09 |
| 3843 | CB | GLN B | 101 | 38.151 | 74.231 | 22.324 | 1.00 | 249.17 |
| 3844 | CG | GLN B | 101 | 37.313 | 73.397 | 23.267 | 1.00 | 249.17 |
| 3845 | CD | GLN B | 101 | 38.163 | 72.685 | 24.297 | 1.00 | 249.17 |
| 3846 | OE1 | GLN B | 101 | 38.643 | 73.292 | 25.255 | 1.00 | 249.17 |
| 3847 | NE2 | GLN B | 101 | 38.370 | 71.395 | 24.094 | 1.00 | 249.17 |
| 3848 | C | GLN B | 101 | 40.159 | 75.687 | 22.000 | 1.00 | 108.09 |
| 3849 | O | GLN B | 101 | 40.186 | 76.880 | 21.723 | 1.00 | 108.09 |
| 3850 | N | PRO B | 102 | 40.996 | 74.815 | 21.422 | 1.00 | 84.30 |
| 3851 | CD | PRO B | 102 | 41.175 | 73.371 | 21.665 | 1.00 | 171.21 |
| 3852 | CA | PRO B | 102 | 41.948 | 75.301 | 20.429 | 1.00 | 84.30 |
| 3853 | CB | PRO B | 102 | 43.006 | 74.212 | 20.431 | 1.00 | 171.21 |
| 3854 | CG | PRO B | 102 | 42.177 | 72.979 | 20.592 | 1.00 | 171.21 |
| 3855 | C | PRO B | 102 | 41.270 | 75.466 | 19.051 | 1.00 | 84.30 |
| 3856 | O | PRO B | 102 | 40.260 | 74.799 | 18.745 | 1.00 | 84.30 |
| 3857 | N | LEU B | 103 | 41.828 | 76.351 | 18.228 | 1.00 | 75.78 |
| 3858 | CA | LEU B | 103 | 41.299 | 76.588 | 16.900 | 1.00 | 75.78 |
| 3859 | CB | LEU B | 103 | 40.437 | 77.830 | 16.910 | 1.00 | 79.40 |
| 3860 | CG | LEU B | 103 | 39.866 | 78.063 | 15.515 | 1.00 | 79.40 |
| 3861 | CD1 | LEU B | 103 | 38.942 | 76.897 | 15.142 | 1.00 | 79.40 |
| 3862 | CD2 | LEU B | 103 | 39.117 | 79.391 | 15.483 | 1.00 | 79.40 |
| 3863 | C | LEU B | 103 | 42.411 | 76.793 | 15.892 | 1.00 | 75.78 |
| 3864 | O | LEU B | 103 | 43.216 | 77.686 | 16.078 | 1.00 | 75.78 |
| 3865 | N | PHE B | 104 | 42.470 | 75.993 | 14.828 | 1.00 | 73.92 |
| 3866 | CA | PHE B | 104 | 43.524 | 76.182 | 13.838 | 1.00 | 73.92 |
| 3867 | CB | PHE B | 104 | 44.441 | 74.953 | 13.752 | 1.00 | 179.34 |
| 3868 | CG | PHE B | 104 | 45.088 | 74.577 | 15.054 | 1.00 | 179.34 |
| 3869 | CD1 | PHE B | 104 | 44.366 | 73.908 | 16.035 | 1.00 | 179.34 |
| 3870 | CD2 | PHE B | 104 | 46.423 | 74.880 | 15.299 | 1.00 | 179.34 |
| 3871 | CE1 | PHE B | 104 | 44.960 | 73.550 | 17.248 | 1.00 | 179.34 |
| 3872 | CE2 | PHE B | 104 | 47.028 | 74.527 | 16.512 | 1.00 | 179.34 |
| 3873 | CZ | PHE B | 104 | 46.295 | 73.857 | 17.485 | 1.00 | 179.34 |
| 3874 | C | PHE B | 104 | 42.958 | 76.472 | 12.448 | 1.00 | 73.92 |
| 3875 | O | PHE B | 104 | 42.121 | 75.717 | 11.947 | 1.00 | 73.92 |
| 3876 | N | LEU B | 105 | 43.387 | 77.581 | 11.838 | 1.00 | 49.92 |
| 3877 | CA | LEU B | 105 | 42.985 | 77.920 | 10.468 | 1.00 | 49.92 |
| 3878 | CB | LEU B | 105 | 42.503 | 79.354 | 10.385 | 1.00 | 78.62 |
| 3879 | CG | LEU B | 105 | 41.409 | 79.667 | 11.381 | 1.00 | 78.62 |
| 3880 | CD1 | LEU B | 105 | 40.828 | 81.064 | 11.137 | 1.00 | 78.62 |
| 3881 | CD2 | LEU B | 105 | 40.368 | 78.612 | 11.223 | 1.00 | 78.62 |
| 3882 | C | LEU B | 105 | 44.224 | 77.773 | 9.580 | 1.00 | 49.92 |
| 3883 | O | LEU B | 105 | 45.327 | 78.095 | 9.991 | 1.00 | 49.92 |
| 3884 | N | ARG B | 106 | 44.051 | 77.317 | 8.355 | 1.00 | 79.70 |
| 3885 | CA | ARG B | 106 | 45.195 | 77.131 | 7.494 | 1.00 | 79.70 |
| 3886 | CB | ARG B | 106 | 45.537 | 75.649 | 7.475 | 1.00 | 126.47 |
| 3887 | CG | ARG B | 106 | 46.633 | 75.284 | 6.526 | 1.00 | 126.47 |
| 3888 | CD | ARG B | 106 | 46.738 | 73.775 | 6.383 | 1.00 | 126.47 |
| 3889 | NE | ARG B | 106 | 47.760 | 73.414 | 5.412 | 1.00 | 126.47 |
| 3890 | CZ | ARG B | 106 | 47.802 | 72.255 | 4.773 | 1.00 | 126.47 |
| 3891 | NH1 | ARG B | 106 | 46.869 | 71.343 | 5.006 | 1.00 | 126.47 |
| 3892 | NH2 | ARG B | 106 | 48.768 | 72.018 | 3.891 | 1.00 | 126.47 |
| 3893 | C | ARG B | 106 | 44.900 | 77.615 | 6.083 | 1.00 | 79.70 |
| 3894 | O | ARG B | 106 | 43.899 | 77.192 | 5.483 | 1.00 | 79.70 |
| 3895 | N | CYS B | 107 | 45.730 | 78.518 | 5.553 | 1.00 | 64.58 |
| 3896 | CA | CYS B | 107 | 45.507 | 78.971 | 4.177 | 1.00 | 64.58 |
| 3897 | C | CYS B | 107 | 46.217 | 77.938 | 3.331 | 1.00 | 64.58 |
| 3898 | O | CYS B | 107 | 47.442 | 77.869 | 3.318 | 1.00 | 64.58 |
| 3899 | CB | CYS B | 107 | 46.087 | 80.353 | 3.919 | 1.00 | 107.35 |
| 3900 | SG | CYS B | 107 | 45.402 | 81.142 | 2.422 | 1.00 | 107.35 |
| 3901 | N | HIS B | 108 | 45.435 | 77.124 | 2.639 | 1.00 | 77.57 |
| 3902 | CA | HIS B | 108 | 45.970 | 76.037 | 1.845 | 1.00 | 77.57 |
| 3903 | CB | HIS B | 108 | 45.151 | 74.790 | 2.131 | 1.00 | 100.22 |
| 3904 | CG | HIS B | 108 | 45.702 | 73.548 | 1.513 | 1.00 | 100.22 |
| 3905 | CD2 | HIS B | 108 | 45.138 | 72.631 | 0.691 | 1.00 | 100.22 |
| 3906 | ND1 | HIS B | 108 | 46.977 | 73.098 | 1.776 | 1.00 | 100.22 |
| 3907 | CE1 | HIS B | 108 | 47.170 | 71.951 | 1.149 | 1.00 | 100.22 |
| 3908 | NE2 | HIS B | 108 | 46.070 | 71.645 | 0.483 | 1.00 | 100.22 |
| 3909 | C | HIS B | 108 | 46.002 | 76.296 | 0.352 | 1.00 | 77.57 |
| 3910 | O | HIS B | 108 | 44.981 | 76.659 | −0.262 | 1.00 | 77.57 |
| 3911 | N | GLY B | 109 | 47.180 | 76.079 | −0.231 | 1.00 | 82.92 |
| 3912 | CA | GLY B | 109 | 47.338 | 76.303 | −1.652 | 1.00 | 82.92 |
| 3913 | C | GLY B | 109 | 47.018 | 75.057 | −2.430 | 1.00 | 82.92 |
| 3914 | O | GLY B | 109 | 47.115 | 73.962 | −1.886 | 1.00 | 82.92 |
| 3915 | N | TRP B | 110 | 46.628 | 75.221 | −3.692 | 1.00 | 89.13 |
| 3916 | CA | TRP B | 110 | 46.294 | 74.086 | −4.536 | 1.00 | 89.13 |
| 3917 | CB | TRP B | 110 | 45.749 | 74.564 | −5.874 | 1.00 | 136.31 |
| 3918 | CG | TRP B | 110 | 45.538 | 73.457 | −6.838 | 1.00 | 136.31 |
| 3919 | CD2 | TRP B | 110 | 44.323 | 72.730 | −7.054 | 1.00 | 136.31 |
| 3920 | CE2 | TRP B | 110 | 44.590 | 71.748 | −8.028 | 1.00 | 136.31 |
| 3921 | CE3 | TRP B | 110 | 43.025 | 72.815 | −6.517 | 1.00 | 136.31 |
| 3922 | CD1 | TRP B | 110 | 46.464 | 72.907 | −7.659 | 1.00 | 136.31 |
| 3923 | NE1 | TRP B | 110 | 45.908 | 71.879 | −8.380 | 1.00 | 136.31 |
| 3924 | CZ2 | TRP B | 110 | 43.615 | 70.853 | −8.484 | 1.00 | 136.31 |
| 3925 | CZ3 | TRP B | 110 | 42.052 | 71.923 | −6.969 | 1.00 | 136.31 |
| 3926 | CH2 | TRP B | 110 | 42.356 | 70.954 | −7.943 | 1.00 | 136.31 |
| 3927 | C | TRP B | 110 | 47.525 | 73.218 | −4.759 | 1.00 | 89.13 |
| 3928 | O | TRP B | 110 | 48.662 | 73.698 | −4.730 | 1.00 | 89.13 |
| 3929 | N | ARG B | 111 | 47.294 | 71.927 | −4.960 | 1.00 | 107.42 |
| 3930 | CA | ARG B | 111 | 48.376 | 70.980 | −5.192 | 1.00 | 107.42 |
| 3931 | CB | ARG B | 111 | 48.900 | 71.128 | −6.598 | 1.00 | 249.40 |
| 3932 | CG | ARG B | 111 | 48.148 | 70.283 | −7.546 | 1.00 | 249.40 |
| 3933 | CD | ARG B | 111 | 48.856 | 70.281 | −8.825 | 1.00 | 249.40 |
| 3934 | NE | ARG B | 111 | 48.857 | 68.940 | −9.379 | 1.00 | 249.40 |
| 3935 | CZ | ARG B | 111 | 49.507 | 67.898 | −8.862 | 1.00 | 249.40 |
| 3936 | NH1 | ARG B | 111 | 50.222 | 68.039 | −7.753 | 1.00 | 249.40 |
| 3937 | NH2 | ARG B | 111 | 49.435 | 66.714 | −9.465 | 1.00 | 249.40 |
| 3938 | C | ARG B | 111 | 49.528 | 71.110 | −4.237 | 1.00 | 107.42 |
| 3939 | O | ARG B | 111 | 50.645 | 70.724 | −4.550 | 1.00 | 107.42 |
| 3940 | N | ASN B | 112 | 49.249 | 71.676 | −3.075 | 1.00 | 103.89 |
| 3941 | CA | ASN B | 112 | 50.250 | 71.869 | −2.050 | 1.00 | 103.89 |
| 3942 | CB | ASN B | 112 | 50.805 | 70.525 | −1.599 | 1.00 | 101.72 |
| 3943 | CG | ASN B | 112 | 51.387 | 70.592 | −0.214 | 1.00 | 101.72 |
| 3944 | OD1 | ASN B | 112 | 51.759 | 71.673 | 0.261 | 1.00 | 101.72 |
| 3945 | ND2 | ASN B | 112 | 51.479 | 69.442 | 0.449 | 1.00 | 101.72 |
| 3946 | C | ASN B | 112 | 51.405 | 72.778 | −2.470 | 1.00 | 103.89 |
| 3947 | O | ASN B | 112 | 52.504 | 72.698 | −1.905 | 1.00 | 103.89 |
| 3948 | N | TRP B | 113 | 51.177 | 73.636 | −3.460 | 1.00 | 84.24 |
| 3949 | CA | TRP B | 113 | 52.232 | 74.553 | −3.854 | 1.00 | 84.24 |
| 3950 | CB | TRP B | 113 | 51.806 | 75.411 | −5.031 | 1.00 | 165.30 |
| 3951 | CG | TRP B | 113 | 51.859 | 74.694 | −6.297 | 1.00 | 165.30 |
| 3952 | CD2 | TRP B | 113 | 50.952 | 74.827 | −7.383 | 1.00 | 165.30 |
| 3953 | CE2 | TRP B | 113 | 51.420 | 73.995 | −8.423 | 1.00 | 165.30 |
| 3954 | CE3 | TRP B | 113 | 49.785 | 75.580 | −7.589 | 1.00 | 165.30 |
| 3955 | CD1 | TRP B | 113 | 52.816 | 73.805 | −6.695 | 1.00 | 165.30 |
| 3956 | NE1 | TRP B | 113 | 52.561 | 73.380 | −7.973 | 1.00 | 165.30 |
| 3957 | CZ2 | TRP B | 113 | 50.763 | 73.890 | −9.652 | 1.00 | 165.30 |
| 3958 | CZ3 | TRP B | 113 | 49.128 | 75.478 | −8.808 | 1.00 | 165.30 |
| 3959 | CH2 | TRP B | 113 | 49.619 | 74.634 | −9.826 | 1.00 | 165.30 |
| 3960 | C | TRP B | 113 | 52.597 | 75.473 | −2.697 | 1.00 | 84.24 |
| 3961 | O | TRP B | 113 | 52.201 | 75.258 | −1.543 | 1.00 | 84.24 |
| 3962 | N | ASP B | 114 | 53.370 | 76.501 | −3.013 | 1.00 | 127.07 |
| 3963 | CA | ASP B | 114 | 53.773 | 77.459 | −2.006 | 1.00 | 127.07 |
| 3964 | CB | ASP B | 114 | 55.289 | 77.629 | −2.007 | 1.00 | 190.00 |
| 3965 | CG | ASP B | 114 | 55.992 | 76.535 | −1.236 | 1.00 | 190.00 |
| 3966 | OD1 | ASP B | 114 | 55.702 | 76.387 | −0.030 | 1.00 | 190.00 |
| 3967 | OD2 | ASP B | 114 | 56.831 | 75.825 | −1.829 | 1.00 | 190.00 |
| 3968 | C | ASP B | 114 | 53.098 | 78.794 | −2.255 | 1.00 | 127.07 |
| 3969 | O | ASP B | 114 | 52.985 | 79.253 | −3.402 | 1.00 | 127.07 |
| 3970 | N | VAL B | 115 | 52.641 | 79.406 | −1.165 | 1.00 | 97.38 |
| 3971 | CA | VAL B | 115 | 51.969 | 80.696 | −1.229 | 1.00 | 97.38 |
| 3972 | CB | VAL B | 115 | 50.571 | 80.635 | −0.623 | 1.00 | 112.10 |
| 3973 | CG1 | VAL B | 115 | 49.833 | 81.905 | −0.946 | 1.00 | 112.10 |
| 3974 | CG2 | VAL B | 115 | 49.830 | 79.436 | −1.155 | 1.00 | 112.10 |
| 3975 | C | VAL B | 115 | 52.767 | 81.723 | −0.451 | 1.00 | 97.38 |
| 3976 | O | VAL B | 115 | 53.333 | 81.428 | 0.613 | 1.00 | 97.38 |
| 3977 | N | TYR B | 116 | 52.804 | 82.940 | −0.985 | 1.00 | 76.30 |
| 3978 | CA | TYR B | 116 | 53.547 | 84.019 | −0.335 | 1.00 | 76.30 |
| 3979 | CB | TYR B | 116 | 54.745 | 84.433 | −1.206 | 1.00 | 116.08 |
| 3980 | CG | TYR B | 116 | 55.758 | 83.328 | −1.431 | 1.00 | 116.08 |
| 3981 | CD1 | TYR B | 116 | 55.720 | 82.529 | −2.581 | 1.00 | 116.08 |
| 3982 | CE1 | TYR B | 116 | 56.634 | 81.486 | −2.770 | 1.00 | 116.08 |
| 3983 | CD2 | TYR B | 116 | 56.736 | 83.061 | −0.476 | 1.00 | 116.08 |
| 3984 | CE2 | TYR B | 116 | 57.657 | 82.024 | −0.650 | 1.00 | 116.08 |
| 3985 | CZ | TYR B | 116 | 57.602 | 81.238 | −1.798 | 1.00 | 116.08 |
| 3986 | OH | TYR B | 116 | 58.498 | 80.201 | −1.964 | 1.00 | 116.08 |
| 3987 | C | TYR B | 116 | 52.654 | 85.227 | −0.059 | 1.00 | 76.30 |
| 3988 | O | TYR B | 116 | 51.502 | 85.276 | −0.514 | 1.00 | 76.30 |
| 3989 | N | LYS B | 117 | 53.193 | 86.190 | 0.692 | 1.00 | 87.81 |
| 3990 | CA | LYS B | 117 | 52.463 | 87.411 | 1.036 | 1.00 | 87.81 |
| 3991 | CB | LYS B | 117 | 52.371 | 88.354 | −0.171 | 1.00 | 224.81 |
| 3992 | CG | LYS B | 117 | 53.560 | 89.283 | −0.373 | 1.00 | 224.81 |
| 3993 | CD | LYS B | 117 | 53.183 | 90.436 | −1.295 | 1.00 | 224.81 |
| 3994 | CE | LYS B | 117 | 52.028 | 91.248 | −0.711 | 1.00 | 224.81 |
| 3995 | NZ | LYS B | 117 | 51.604 | 92.373 | −1.593 | 1.00 | 224.81 |
| 3996 | C | LYS B | 117 | 51.059 | 87.049 | 1.489 | 1.00 | 87.81 |
| 3997 | O | LYS B | 117 | 50.060 | 87.542 | 0.946 | 1.00 | 87.81 |
| 3998 | N | VAL B | 118 | 50.983 | 86.194 | 2.498 | 1.00 | 60.09 |
| 3999 | CA | VAL B | 118 | 49.696 | 85.737 | 2.994 | 1.00 | 60.09 |
| 4000 | CB | VAL B | 118 | 49.815 | 84.344 | 3.577 | 1.00 | 85.68 |
| 4001 | CG1 | VAL B | 118 | 48.782 | 84.141 | 4.647 | 1.00 | 85.68 |
| 4002 | CG2 | VAL B | 118 | 49.604 | 83.325 | 2.485 | 1.00 | 85.68 |
| 4003 | C | VAL B | 118 | 49.066 | 86.622 | 4.034 | 1.00 | 60.09 |
| 4004 | O | VAL B | 118 | 49.752 | 87.066 | 4.963 | 1.00 | 60.09 |
| 4005 | N | ILE B | 119 | 47.753 | 86.837 | 3.901 | 1.00 | 64.26 |
| 4006 | CA | ILE B | 119 | 47.003 | 87.686 | 4.830 | 1.00 | 64.26 |
| 4007 | CB | ILE B | 119 | 46.704 | 89.027 | 4.196 | 1.00 | 68.56 |
| 4008 | CG2 | ILE B | 119 | 46.039 | 89.920 | 5.184 | 1.00 | 68.56 |
| 4009 | CG1 | ILE B | 119 | 47.998 | 89.654 | 3.705 | 1.00 | 68.56 |
| 4010 | CD1 | ILE B | 119 | 47.766 | 90.828 | 2.843 | 1.00 | 68.56 |
| 4011 | C | ILE B | 119 | 45.672 | 87.052 | 5.173 | 1.00 | 64.26 |
| 4012 | O | ILE B | 119 | 44.890 | 86.771 | 4.259 | 1.00 | 64.26 |
| 4013 | N | TYR B | 120 | 45.402 | 86.803 | 6.458 | 1.00 | 74.17 |
| 4014 | CA | TYR B | 120 | 44.110 | 86.216 | 6.816 | 1.00 | 74.17 |
| 4015 | CB | TYR B | 120 | 44.176 | 85.345 | 8.066 | 1.00 | 67.28 |
| 4016 | CG | TYR B | 120 | 44.901 | 84.067 | 7.887 | 1.00 | 67.28 |
| 4017 | CD1 | TYR B | 120 | 46.269 | 84.024 | 8.002 | 1.00 | 67.28 |
| 4018 | CE1 | TYR B | 120 | 46.975 | 82.840 | 7.818 | 1.00 | 67.28 |
| 4019 | CD2 | TYR B | 120 | 44.225 | 82.896 | 7.582 | 1.00 | 67.28 |
| 4020 | CE2 | TYR B | 120 | 44.909 | 81.695 | 7.390 | 1.00 | 67.28 |
| 4021 | CZ | TYR B | 120 | 46.286 | 81.680 | 7.511 | 1.00 | 67.28 |
| 4022 | OH | TYR B | 120 | 46.966 | 80.507 | 7.340 | 1.00 | 67.28 |
| 4023 | C | TYR B | 120 | 43.185 | 87.348 | 7.125 | 1.00 | 74.17 |
| 4024 | O | TYR B | 120 | 43.613 | 88.351 | 7.669 | 1.00 | 74.17 |
| 4025 | N | TYR B | 121 | 41.916 | 87.180 | 6.799 | 1.00 | 60.66 |
| 4026 | CA | TYR B | 121 | 40.938 | 88.213 | 7.080 | 1.00 | 60.66 |
| 4027 | CB | TYR B | 121 | 40.355 | 88.760 | 5.776 | 1.00 | 108.81 |
| 4028 | CG | TYR B | 121 | 41.299 | 89.557 | 4.908 | 1.00 | 108.81 |
| 4029 | CD1 | TYR B | 121 | 42.398 | 88.961 | 4.308 | 1.00 | 108.81 |
| 4030 | CE1 | TYR B | 121 | 43.239 | 89.678 | 3.449 | 1.00 | 108.81 |
| 4031 | CD2 | TYR B | 121 | 41.058 | 90.900 | 4.640 | 1.00 | 108.81 |
| 4032 | CE2 | TYR B | 121 | 41.890 | 91.629 | 3.788 | 1.00 | 108.81 |
| 4033 | CZ | TYR B | 121 | 42.976 | 91.009 | 3.195 | 1.00 | 108.81 |
| 4034 | OH | TYR B | 121 | 43.794 | 91.710 | 2.340 | 1.00 | 108.81 |
| 4035 | C | TYR B | 121 | 39.781 | 87.692 | 7.936 | 1.00 | 60.66 |
| 4036 | O | TYR B | 121 | 39.301 | 86.560 | 7.736 | 1.00 | 60.66 |
| 4037 | N | LYS B | 122 | 39.332 | 88.510 | 8.885 | 1.00 | 76.13 |
| 4038 | CA | LYS B | 122 | 38.194 | 88.138 | 9.715 | 1.00 | 76.13 |
| 4039 | CB | LYS B | 122 | 38.594 | 87.874 | 11.168 | 1.00 | 102.31 |
| 4040 | CG | LYS B | 122 | 37.410 | 87.462 | 12.032 | 1.00 | 102.31 |
| 4041 | CD | LYS B | 122 | 37.738 | 87.523 | 13.489 | 1.00 | 102.31 |
| 4042 | CE | LYS B | 122 | 36.509 | 87.285 | 14.327 | 1.00 | 102.31 |
| 4043 | NZ | LYS B | 122 | 36.834 | 87.504 | 15.762 | 1.00 | 102.31 |
| 4044 | C | LYS B | 122 | 37.200 | 89.289 | 9.679 | 1.00 | 76.13 |
| 4045 | O | LYS B | 122 | 37.507 | 90.390 | 10.145 | 1.00 | 76.13 |
| 4046 | N | ASP B | 123 | 36.013 | 89.034 | 9.131 | 1.00 | 98.55 |
| 4047 | CA | ASP B | 123 | 34.968 | 90.049 | 9.023 | 1.00 | 98.55 |
| 4043 | CB | ASP B | 123 | 34.492 | 90.473 | 10.414 | 1.00 | 136.85 |
| 4049 | CG | ASP B | 123 | 33.604 | 89.429 | 11.059 | 1.00 | 136.85 |
| 4050 | OD1 | ASP B | 123 | 32.692 | 88.925 | 10.363 | 1.00 | 136.85 |
| 4051 | OD2 | ASP B | 123 | 33.810 | 89.122 | 12.256 | 1.00 | 136.85 |
| 4052 | C | ASP B | 123 | 35.420 | 91.268 | 8.217 | 1.00 | 98.55 |
| 4053 | O | ASP B | 123 | 35.168 | 92.418 | 8.597 | 1.00 | 98.55 |
| 4054 | N | GLY B | 124 | 36.094 | 90.997 | 7.099 | 1.00 | 109.74 |
| 4055 | CA | GLY B | 124 | 36.578 | 92.050 | 6.224 | 1.00 | 109.74 |
| 4056 | C | GLY B | 124 | 37.817 | 92.800 | 6.688 | 1.00 | 109.74 |
| 4057 | O | GLY B | 124 | 38.371 | 93.600 | 5.938 | 1.00 | 109.74 |
| 4058 | N | GLU B | 125 | 38.269 | 92.542 | 7.911 | 1.00 | 80.11 |
| 4059 | CA | GLU B | 125 | 39.438 | 93.230 | 8.468 | 1.00 | 80.11 |
| 4060 | CB | GLU B | 125 | 39.276 | 93.432 | 9.990 | 1.00 | 173.35 |
| 4061 | CG | GLU B | 125 | 38.192 | 94.412 | 10.446 | 1.00 | 173.35 |
| 4062 | CD | GLU B | 125 | 38.621 | 95.865 | 10.344 | 1.00 | 173.35 |
| 4063 | OE1 | GLU B | 125 | 39.591 | 96.256 | 11.030 | 1.00 | 173.35 |
| 4064 | OE2 | GLU B | 125 | 37.982 | 96.615 | 9.577 | 1.00 | 173.35 |
| 4065 | C | GLU B | 125 | 40.723 | 92.462 | 8.243 | 1.00 | 80.11 |
| 4066 | O | GLU B | 125 | 40.728 | 91.235 | 8.308 | 1.00 | 80.11 |
| 4067 | N | ALA B | 126 | 41.817 | 93.171 | 7.986 | 1.00 | 116.19 |
| 4068 | CA | ALA B | 126 | 43.101 | 92.501 | 7.826 | 1.00 | 116.19 |
| 4069 | CB | ALA B | 126 | 44.165 | 93.513 | 7.450 | 1.00 | 157.65 |
| 4070 | C | ALA B | 126 | 43.385 | 91.901 | 9.216 | 1.00 | 116.19 |
| 4071 | O | ALA B | 126 | 43.051 | 92.516 | 10.227 | 1.00 | 116.19 |
| 4072 | N | LEU B | 127 | 43.985 | 90.715 | 9.286 | 1.00 | 101.69 |
| 4073 | CA | LEU B | 127 | 44.246 | 90.109 | 10.586 | 1.00 | 101.69 |
| 4074 | CB | LEU B | 127 | 43.383 | 88.875 | 10.761 | 1.00 | 85.89 |
| 4075 | CG | LEU B | 127 | 43.207 | 88.660 | 12.259 | 1.00 | 85.89 |
| 4076 | CD1 | LEU B | 127 | 42.594 | 89.929 | 12.853 | 1.00 | 85.89 |
| 4077 | CD2 | LEU B | 127 | 42.337 | 87.450 | 12.551 | 1.00 | 85.89 |
| 4078 | C | LEU B | 127 | 45.696 | 89.750 | 10.902 | 1.00 | 101.69 |
| 4079 | O | LEU B | 127 | 46.240 | 90.197 | 11.910 | 1.00 | 101.69 |
| 4080 | N | LYS B | 128 | 46.306 | 88.916 | 10.070 | 1.00 | 84.42 |
| 4081 | CA | LYS B | 128 | 47.701 | 88.530 | 10.256 | 1.00 | 84.42 |
| 4082 | CB | LYS B | 128 | 47.794 | 87.118 | 10.847 | 1.00 | 200.20 |
| 4083 | CG | LYS B | 128 | 47.160 | 86.955 | 12.220 | 1.00 | 200.20 |
| 4084 | CD | LYS B | 128 | 47.968 | 87.634 | 13.317 | 1.00 | 200.20 |
| 4085 | CE | LYS B | 128 | 47.352 | 87.350 | 14.684 | 1.00 | 200.20 |
| 4086 | NZ | LYS B | 128 | 48.172 | 87.864 | 15.817 | 1.00 | 200.20 |
| 4087 | C | LYS B | 128 | 48.360 | 88.558 | 8.877 | 1.00 | 84.42 |
| 4088 | O | LYS B | 128 | 47.675 | 88.404 | 7.853 | 1.00 | 84.42 |
| 4089 | N | TYR B | 129 | 49.675 | 88.750 | 8.839 | 1.00 | 107.40 |
| 4090 | CA | TYR B | 129 | 50.387 | 88.773 | 7.566 | 1.00 | 107.40 |
| 4091 | CB | TYR B | 129 | 50.519 | 90.208 | 7.067 | 1.00 | 112.63 |
| 4092 | CG | TYR B | 129 | 51.618 | 90.368 | 6.043 | 1.00 | 112.63 |
| 4093 | CD1 | TYR B | 129 | 51.417 | 90.021 | 4.712 | 1.00 | 112.63 |
| 4094 | CE1 | TYR B | 129 | 52.442 | 90.116 | 3.781 | 1.00 | 112.63 |
| 4095 | CD2 | TYR B | 129 | 52.879 | 90.814 | 6.422 | 1.00 | 112.63 |
| 4096 | CE2 | TYR B | 129 | 53.915 | 90.911 | 5.502 | 1.00 | 112.63 |
| 4097 | CZ | TYR B | 129 | 53.693 | 90.562 | 4.183 | 1.00 | 112.63 |
| 4098 | OH | TYR B | 129 | 54.719 | 90.665 | 3.260 | 1.00 | 112.63 |
| 4099 | C | TYR B | 129 | 51.779 | 88.146 | 7.621 | 1.00 | 107.40 |
| 4100 | O | TYR B | 129 | 52.518 | 88.373 | 8.575 | 1.00 | 107.40 |
| 4101 | N | TRP B | 130 | 52.138 | 87.376 | 6.587 | 1.00 | 87.42 |
| 4102 | CA | TRP B | 130 | 53.454 | 86.735 | 6.524 | 1.00 | 87.42 |
| 4103 | CB | TRP B | 130 | 53.400 | 85.311 | 7.090 | 1.00 | 190.57 |
| 4104 | CG | TRP B | 130 | 52.744 | 85.171 | 8.423 | 1.00 | 190.57 |
| 4105 | CD2 | TRP B | 130 | 53.401 | 84.954 | 9.674 | 1.00 | 190.57 |
| 4106 | CE2 | TRP B | 130 | 52.392 | 84.829 | 10.656 | 1.00 | 190.57 |
| 4107 | CE3 | TRP B | 130 | 54.746 | 84.861 | 10.062 | 1.00 | 190.57 |
| 4108 | CD1 | TRP B | 130 | 51.406 | 85.166 | 8.687 | 1.00 | 190.57 |
| 4109 | NE1 | TRP B | 130 | 51.185 | 84.959 | 10.025 | 1.00 | 190.57 |
| 4110 | CZ2 | TRP B | 130 | 52.686 | 84.607 | 12.010 | 1.00 | 190.57 |
| 4111 | CZ3 | TRP B | 130 | 55.041 | 84.641 | 11.412 | 1.00 | 190.57 |
| 4112 | CH2 | TRP B | 130 | 54.011 | 84.510 | 12.366 | 1.00 | 190.57 |
| 4113 | C | TRP B | 130 | 53.968 | 86.652 | 5.085 | 1.00 | 87.42 |
| 4114 | O | TRP B | 130 | 53.209 | 86.847 | 4.127 | 1.00 | 87.42 |
| 4115 | N | TYR B | 131 | 55.259 | 86.362 | 4.940 | 1.00 | 97.57 |
| 4116 | CA | TYR B | 131 | 55.848 | 86.212 | 3.621 | 1.00 | 97.57 |
| 4117 | CB | TYR B | 131 | 57.339 | 86.504 | 3.647 | 1.00 | 249.42 |
| 4118 | CG | TYR B | 131 | 57.881 | 86.590 | 2.250 | 1.00 | 249.42 |
| 4119 | CD1 | TYR B | 131 | 57.611 | 87.707 | 1.453 | 1.00 | 249.42 |
| 4120 | CE1 | TYR B | 131 | 57.982 | 87.746 | 0.133 | 1.00 | 249.42 |
| 4121 | CD2 | TYR B | 131 | 58.554 | 85.514 | 1.676 | 1.00 | 249.42 |
| 4122 | CE2 | TYR B | 131 | 58.929 | 85.544 | 0.355 | 1.00 | 249.42 |
| 4123 | CZ | TYR B | 131 | 58.628 | 86.660 | −0.404 | 1.00 | 249.42 |
| 4124 | OH | TYR B | 131 | 58.902 | 86.675 | −1.726 | 1.00 | 249.42 |
| 4125 | C | TYR B | 131 | 55.619 | 84.751 | 3.231 | 1.00 | 97.57 |
| 4126 | O | TYR B | 131 | 54.661 | 84.439 | 2.509 | 1.00 | 97.57 |
| 4127 | N | GLU B | 132 | 56.517 | 83.862 | 3.669 | 1.00 | 249.33 |
| 4128 | CA | GLU B | 132 | 56.333 | 82.428 | 3.432 | 1.00 | 249.33 |
| 4129 | CB | GLU B | 132 | 57.528 | 81.602 | 3.941 | 1.00 | 249.46 |
| 4130 | CG | GLU B | 132 | 58.788 | 81.623 | 3.066 | 1.00 | 249.46 |
| 4131 | CD | GLU B | 132 | 59.162 | 80.239 | 2.532 | 1.00 | 249.46 |
| 4132 | OE1 | GLU B | 132 | 58.693 | 79.231 | 3.102 | 1.00 | 249.46 |
| 4133 | OE2 | GLU B | 132 | 59.935 | 80.161 | 1.551 | 1.00 | 249.46 |
| 4134 | C | GLU B | 132 | 55.158 | 82.276 | 4.384 | 1.00 | 249.33 |
| 4135 | O | GLU B | 132 | 55.259 | 82.685 | 5.543 | 1.00 | 249.33 |
| 4136 | N | ASN B | 133 | 54.047 | 81.711 | 3.924 | 1.00 | 134.43 |
| 4137 | CA | ASN B | 133 | 52.884 | 81.642 | 4.798 | 1.00 | 134.43 |
| 4138 | CB | ASN B | 133 | 51.649 | 81.176 | 4.033 | 1.00 | 135.42 |
| 4139 | CG | ASN B | 133 | 51.534 | 79.690 | 3.981 | 1.00 | 135.42 |
| 4140 | OD1 | ASN B | 133 | 52.439 | 78.995 | 3.617 | 1.00 | 135.42 |
| 4141 | ND2 | ASN B | 133 | 50.358 | 79.176 | 4.337 | 1.00 | 135.42 |
| 4142 | C | ASN B | 133 | 53.019 | 80.848 | 6.080 | 1.00 | 134.43 |
| 4143 | O | ASN B | 133 | 54.026 | 80.185 | 6.338 | 1.00 | 134.43 |
| 4144 | N | HIS B | 134 | 51.962 | 80.932 | 6.875 | 1.00 | 135.01 |
| 4145 | CA | HIS B | 134 | 51.905 | 80.302 | 8.174 | 1.00 | 135.01 |
| 4146 | CB | HIS B | 134 | 52.150 | 81.381 | 9.224 | 1.00 | 225.09 |
| 4147 | CG | HIS B | 134 | 52.262 | 80.865 | 10.622 | 1.00 | 225.09 |
| 4148 | CD2 | HIS B | 134 | 51.493 | 81.086 | 11.714 | 1.00 | 225.09 |
| 4149 | ND1 | HIS B | 134 | 53.283 | 80.038 | 11.032 | 1.00 | 225.09 |
| 4150 | CE1 | HIS B | 134 | 53.140 | 79.772 | 12.320 | 1.00 | 225.09 |
| 4151 | NE2 | HIS B | 134 | 52.063 | 80.396 | 12.756 | 1.00 | 225.09 |
| 4152 | C | HIS B | 134 | 50.531 | 79.673 | 8.355 | 1.00 | 135.01 |
| 4153 | O | HIS B | 134 | 49.789 | 79.494 | 7.385 | 1.00 | 135.01 |
| 4154 | N | ASN B | 135 | 50.197 | 79.346 | 9.601 | 1.00 | 105.44 |
| 4155 | CA | ASN B | 135 | 48.928 | 78.730 | 9.922 | 1.00 | 105.44 |
| 4156 | CB | ASN B | 135 | 49.090 | 77.209 | 10.001 | 1.00 | 235.21 |
| 4157 | CG | ASN B | 135 | 49.415 | 76.600 | 8.653 | 1.00 | 235.21 |
| 4158 | OD1 | ASN B | 135 | 48.779 | 76.948 | 7.657 | 1.00 | 235.21 |
| 4159 | ND2 | ASN B | 135 | 50.383 | 75.689 | 8.604 | 1.00 | 235.21 |
| 4160 | C | ASN B | 135 | 48.399 | 79.280 | 11.223 | 1.00 | 105.44 |
| 4161 | O | ASN B | 135 | 48.611 | 78.700 | 12.279 | 1.00 | 105.44 |
| 4162 | N | ILE B | 136 | 47.718 | 80.417 | 11.134 | 1.00 | 66.02 |
| 4163 | CA | ILE B | 136 | 47.123 | 81.076 | 12.304 | 1.00 | 66.02 |
| 4164 | CB | ILE B | 136 | 46.015 | 82.066 | 11.860 | 1.00 | 141.15 |
| 4165 | CG2 | ILE B | 136 | 45.045 | 81.385 | 10.926 | 1.00 | 141.15 |
| 4166 | CG1 | ILE B | 136 | 45.283 | 82.620 | 13.068 | 1.00 | 141.15 |
| 4167 | CD1 | ILE B | 136 | 44.290 | 83.673 | 12.695 | 1.00 | 141.15 |
| 4168 | C | ILE B | 136 | 46.555 | 80.069 | 13.307 | 1.00 | 66.02 |
| 4169 | O | ILE B | 136 | 45.602 | 79.339 | 13.022 | 1.00 | 66.02 |
| 4170 | N | SER B | 137 | 47.160 | 80.045 | 14.486 | 1.00 | 95.21 |
| 4171 | CA | SER B | 137 | 46.768 | 79.114 | 15.538 | 1.00 | 95.21 |
| 4172 | CB | SER B | 137 | 47.968 | 78.233 | 15.897 | 1.00 | 97.51 |
| 4173 | OG | SER B | 137 | 47.742 | 77.531 | 17.105 | 1.00 | 97.51 |
| 4174 | C | SER B | 137 | 46.218 | 79.776 | 16.807 | 1.00 | 95.21 |
| 4175 | O | SER B | 137 | 46.625 | 80.869 | 17.185 | 1.00 | 95.21 |
| 4176 | N | ILE B | 138 | 45.298 | 79.095 | 17.472 | 1.00 | 236.44 |
| 4177 | CA | ILE B | 138 | 44.698 | 79.625 | 18.688 | 1.00 | 236.44 |
| 4178 | CB | ILE B | 138 | 43.295 | 80.153 | 18.420 | 1.00 | 113.67 |
| 4179 | CG2 | ILE B | 138 | 42.601 | 80.445 | 19.737 | 1.00 | 113.67 |
| 4180 | CG1 | ILE B | 138 | 43.363 | 81.397 | 17.533 | 1.00 | 113.67 |
| 4181 | CD1 | ILE B | 138 | 42.021 | 81.787 | 16.948 | 1.00 | 113.67 |
| 4182 | C | ILE B | 138 | 44.580 | 78.558 | 19.761 | 1.00 | 236.44 |
| 4183 | O | ILE B | 138 | 43.936 | 77.531 | 19.555 | 1.00 | 236.44 |
| 4184 | N | THR B | 139 | 45.180 | 78.816 | 20.915 | 1.00 | 117.60 |
| 4185 | CA | THR B | 139 | 45.131 | 77.865 | 22.018 | 1.00 | 117.60 |
| 4186 | CB | THR B | 139 | 46.259 | 78.143 | 23.020 | 1.00 | 212.12 |
| 4187 | OG1 | THR B | 139 | 46.227 | 79.521 | 23.406 | 1.00 | 212.12 |
| 4188 | CG2 | THR B | 139 | 47.609 | 77.830 | 22.390 | 1.00 | 212.12 |
| 4189 | C | THR B | 139 | 43.780 | 77.942 | 22.733 | 1.00 | 117.60 |
| 4190 | O | THR B | 139 | 42.898 | 77.092 | 22.541 | 1.00 | 117.60 |
| 4191 | N | ASN B | 140 | 43.633 | 78.960 | 23.573 | 1.00 | 147.27 |
| 4192 | CA | ASN B | 140 | 42.396 | 79.189 | 24.308 | 1.00 | 147.27 |
| 4193 | CB | ASN B | 140 | 42.685 | 79.890 | 25.631 | 1.00 | 247.00 |
| 4194 | CG | ASN B | 140 | 41.426 | 80.263 | 26.369 | 1.00 | 247.00 |
| 4195 | OD1 | ASN B | 140 | 40.498 | 80.833 | 25.789 | 1.00 | 247.00 |
| 4196 | ND2 | ASN B | 140 | 41.395 | 79.951 | 27.658 | 1.00 | 247.00 |
| 4197 | C | ASN B | 140 | 41.572 | 80.103 | 23.421 | 1.00 | 147.27 |
| 4198 | O | ASN B | 140 | 42.004 | 81.206 | 23.092 | 1.00 | 147.27 |
| 4199 | N | ALA B | 141 | 40.383 | 79.652 | 23.048 | 1.00 | 102.95 |
| 4200 | CA | ALA B | 141 | 39.537 | 80.432 | 22.163 | 1.00 | 102.95 |
| 4201 | CB | ALA B | 141 | 38.862 | 79.519 | 21.162 | 1.00 | 101.29 |
| 4202 | C | ALA B | 141 | 38.493 | 81.273 | 22.850 | 1.00 | 102.95 |
| 4203 | O | ALA B | 141 | 37.722 | 80.782 | 23.680 | 1.00 | 102.95 |
| 4204 | N | THR B | 142 | 38.458 | 82.546 | 22.474 | 1.00 | 139.44 |
| 4205 | CA | THR B | 142 | 37.495 | 83.478 | 23.021 | 1.00 | 139.44 |
| 4206 | CB | THR B | 142 | 38.055 | 84.903 | 22.959 | 1.00 | 140.37 |
| 4207 | OG1 | THR B | 142 | 39.366 | 84.918 | 23.544 | 1.00 | 140.37 |
| 4208 | CG2 | THR B | 142 | 37.166 | 85.858 | 23.725 | 1.00 | 140.37 |
| 4209 | C | THR B | 142 | 36.220 | 83.361 | 22.184 | 1.00 | 139.44 |
| 4210 | O | THR B | 142 | 36.216 | 82.675 | 21.160 | 1.00 | 139.44 |
| 4211 | N | VAL B | 143 | 35.132 | 83.988 | 22.623 | 1.00 | 168.09 |
| 4212 | CA | VAL B | 143 | 33.884 | 83.922 | 21.866 | 1.00 | 168.09 |
| 4213 | CB | VAL B | 143 | 32.633 | 84.134 | 22.755 | 1.00 | 243.26 |
| 4214 | CG1 | VAL B | 143 | 32.616 | 85.552 | 23.305 | 1.00 | 243.26 |
| 4215 | CG2 | VAL B | 143 | 31.366 | 83.867 | 21.946 | 1.00 | 243.26 |
| 4216 | C | VAL B | 143 | 33.925 | 85.029 | 20.834 | 1.00 | 168.09 |
| 4217 | O | VAL B | 143 | 33.150 | 85.035 | 19.878 | 1.00 | 168.09 |
| 4218 | N | GLU B | 144 | 34.839 | 85.971 | 21.035 | 1.00 | 126.58 |
| 4219 | CA | GLU B | 144 | 34.975 | 87.081 | 20.108 | 1.00 | 126.58 |
| 4220 | CB | GLU B | 144 | 35.750 | 88.229 | 20.751 | 1.00 | 249.26 |
| 4221 | CG | GLU B | 144 | 35.040 | 88.850 | 21.940 | 1.00 | 249.26 |
| 4222 | CD | GLU B | 144 | 35.771 | 88.603 | 23.243 | 1.00 | 249.26 |
| 4223 | OE1 | GLU B | 144 | 36.942 | 89.024 | 23.353 | 1.00 | 249.26 |
| 4224 | OE2 | GLU B | 144 | 35.181 | 87.989 | 24.157 | 1.00 | 249.26 |
| 4225 | C | GLU B | 144 | 35.673 | 86.623 | 18.840 | 1.00 | 126.58 |
| 4226 | O | GLU B | 144 | 35.633 | 87.305 | 17.826 | 1.00 | 126.58 |
| 4227 | N | ASP B | 145 | 36.307 | 85.457 | 18.903 | 1.00 | 80.30 |
| 4228 | CA | ASP B | 145 | 36.997 | 84.893 | 17.752 | 1.00 | 80.30 |
| 4229 | CB | ASP B | 145 | 37.911 | 83.753 | 18.189 | 1.00 | 204.44 |
| 4230 | CG | ASP B | 145 | 39.132 | 84.250 | 18.918 | 1.00 | 204.44 |
| 4231 | OD1 | ASP B | 145 | 39.896 | 85.030 | 18.310 | 1.00 | 204.44 |
| 4232 | OD2 | ASP B | 145 | 39.326 | 83.869 | 20.092 | 1.00 | 204.44 |
| 4233 | C | ASP B | 145 | 36.026 | 84.395 | 16.699 | 1.00 | 80.30 |
| 4234 | O | ASP B | 145 | 36.421 | 84.133 | 15.569 | 1.00 | 80.30 |
| 4235 | N | SER B | 146 | 34.755 | 84.263 | 17.062 | 1.00 | 110.67 |
| 4236 | CA | SER B | 146 | 33.761 | 83.800 | 16.108 | 1.00 | 110.67 |
| 4237 | CB | SER B | 146 | 32.421 | 83.569 | 16.815 | 1.00 | 166.23 |
| 4238 | OG | SER B | 146 | 32.547 | 82.573 | 17.814 | 1.00 | 166.23 |
| 4239 | C | SER B | 146 | 33.646 | 84.870 | 15.022 | 1.00 | 110.67 |
| 4240 | O | SER B | 146 | 33.736 | 86.063 | 15.302 | 1.00 | 110.67 |
| 4241 | N | GLY B | 147 | 33.487 | 84.436 | 13.778 | 1.00 | 85.62 |
| 4242 | CA | GLY B | 147 | 33.375 | 85.365 | 12.670 | 1.00 | 85.62 |
| 4243 | C | GLY B | 147 | 33.473 | 84.635 | 11.353 | 1.00 | 85.62 |
| 4244 | O | GLY B | 147 | 33.311 | 83.417 | 11.301 | 1.00 | 85.62 |
| 4245 | N | THR B | 148 | 33.737 | 85.372 | 10.279 | 1.00 | 63.43 |
| 4246 | CA | THR B | 148 | 33.851 | 84.756 | 8.952 | 1.00 | 63.43 |
| 4247 | CB | THR B | 148 | 32.729 | 85.244 | 7.991 | 1.00 | 111.42 |
| 4248 | OG1 | THR B | 148 | 33.253 | 86.223 | 7.103 | 1.00 | 111.42 |
| 4249 | CG2 | THR B | 148 | 31.609 | 85.879 | 8.767 | 1.00 | 111.42 |
| 4250 | C | THR B | 148 | 35.227 | 85.092 | 8.397 | 1.00 | 63.43 |
| 4251 | O | THR B | 148 | 35.568 | 86.244 | 8.176 | 1.00 | 63.43 |
| 4252 | N | TYR B | 149 | 36.024 | 84.069 | 8.183 | 1.00 | 61.50 |
| 4253 | CA | TYR B | 149 | 37.366 | 84.273 | 7.705 | 1.00 | 61.50 |
| 4254 | CB | TYR B | 149 | 38.298 | 83.380 | 8.514 | 1.00 | 61.70 |
| 4255 | CG | TYR B | 149 | 38.353 | 83.629 | 10.007 | 1.00 | 61.70 |
| 4256 | CD1 | TYR B | 149 | 37.273 | 83.361 | 10.839 | 1.00 | 61.70 |
| 4257 | CE1 | TYR B | 149 | 37.373 | 83.572 | 12.217 | 1.00 | 61.70 |
| 4258 | CD2 | TYR B | 149 | 39.514 | 84.106 | 10.587 | 1.00 | 61.70 |
| 4259 | CE2 | TYR B | 149 | 39.626 | 84.317 | 11.939 | 1.00 | 61.70 |
| 4260 | CZ | TYR B | 149 | 38.571 | 84.060 | 12.757 | 1.00 | 61.70 |
| 4261 | OH | TYR B | 149 | 38.744 | 84.327 | 14.104 | 1.00 | 61.70 |
| 4262 | C | TYR B | 149 | 37.540 | 83.934 | 6.223 | 1.00 | 61.50 |
| 4263 | O | TYR B | 149 | 36.666 | 83.307 | 5.605 | 1.00 | 61.50 |
| 4264 | N | TYR B | 150 | 38.674 | 84.372 | 5.669 | 1.00 | 57.66 |
| 4265 | CA | TYR B | 150 | 39.090 | 84.071 | 4.302 | 1.00 | 57.66 |
| 4266 | CB | TYR B | 150 | 38.189 | 84.760 | 3.264 | 1.00 | 101.41 |
| 4267 | CG | TYR B | 150 | 38.386 | 86.234 | 3.018 | 1.00 | 101.41 |
| 4268 | CD1 | TYR B | 150 | 39.493 | 86.704 | 2.338 | 1.00 | 101.41 |
| 4269 | CE1 | TYR B | 150 | 39.653 | 88.065 | 2.074 | 1.00 | 101.41 |
| 4270 | CD2 | TYR B | 150 | 37.435 | 87.160 | 3.428 | 1.00 | 101.41 |
| 4271 | CE2 | TYR B | 150 | 37.582 | 88.520 | 3.163 | 1.00 | 101.41 |
| 4272 | CZ | TYR B | 150 | 38.693 | 88.967 | 2.489 | 1.00 | 101.41 |
| 4273 | OH | TYR B | 150 | 38.855 | 90.316 | 2.245 | 1.00 | 101.41 |
| 4274 | C | TYR B | 150 | 40.539 | 84.536 | 4.251 | 1.00 | 57.66 |
| 4275 | O | TYR B | 150 | 40.952 | 85.331 | 5.109 | 1.00 | 57.66 |
| 4276 | N | CYS B | 151 | 41.340 | 84.020 | 3.318 | 1.00 | 78.79 |
| 4277 | CA | CYS B | 151 | 42.736 | 84.458 | 3.227 | 1.00 | 78.79 |
| 4278 | C | CYS B | 151 | 43.124 | 84.856 | 1.804 | 1.00 | 78.79 |
| 4279 | O | CYS B | 151 | 42.464 | 84.461 | 0.849 | 1.00 | 78.79 |
| 4280 | CB | CYS B | 151 | 43.680 | 83.369 | 3.736 | 1.00 | 103.97 |
| 4281 | SG | CYS B | 151 | 43.510 | 81.730 | 2.951 | 1.00 | 103.97 |
| 4282 | N | THR B | 152 | 44.174 | 85.668 | 1.673 | 1.00 | 109.05 |
| 4283 | CA | THR B | 152 | 44.663 | 86.107 | 0.370 | 1.00 | 109.05 |
| 4284 | CB | THR B | 152 | 44.524 | 87.630 | 0.206 | 1.00 | 169.15 |
| 4285 | OG1 | THR B | 152 | 45.394 | 88.296 | 1.133 | 1.00 | 169.15 |
| 4286 | CG2 | THR B | 152 | 43.097 | 88.056 | 0.475 | 1.00 | 169.15 |
| 4287 | C | THR B | 152 | 46.139 | 85.728 | 0.297 | 1.00 | 109.05 |
| 4288 | O | THR B | 152 | 46.839 | 85.740 | 1.317 | 1.00 | 109.05 |
| 4289 | N | GLY B | 153 | 46.611 | 65.381 | −0.898 | 1.00 | 135.93 |
| 4290 | CA | GLY B | 153 | 48.007 | 85.000 | −1.054 | 1.00 | 135.93 |
| 4291 | C | GLY B | 153 | 48.447 | 85.023 | −2.501 | 1.00 | 135.93 |
| 4292 | O | GLY B | 153 | 47.618 | 85.025 | −3.404 | 1.00 | 135.93 |
| 4293 | N | LYS B | 154 | 49.751 | 85.038 | −2.734 | 1.00 | 88.42 |
| 4294 | CA | LYS B | 154 | 50.252 | 85.068 | −4.096 | 1.00 | 88.42 |
| 4295 | CB | LYS B | 154 | 51.392 | 86.090 | −4.216 | 1.00 | 187.09 |
| 4296 | CG | LYS B | 154 | 51.920 | 86.317 | −5.630 | 1.00 | 187.09 |
| 4297 | CD | LYS B | 154 | 53.003 | 87.393 | −5.619 | 1.00 | 187.09 |
| 4298 | CE | LYS B | 154 | 53.634 | 87.579 | −6.975 | 1.00 | 187.09 |
| 4299 | NZ | LYS B | 154 | 54.766 | 88.490 | −6.839 | 1.00 | 187.09 |
| 4300 | C | LYS B | 154 | 50.744 | 83.673 | −4.404 | 1.00 | 88.42 |
| 4301 | O | LYS B | 154 | 51.450 | 83.057 | −3.592 | 1.00 | 88.42 |
| 4302 | N | VAL B | 155 | 50.332 | 83.166 | −5.561 | 1.00 | 135.91 |
| 4303 | CA | VAL B | 155 | 50.742 | 81.845 | −6.018 | 1.00 | 135.91 |
| 4304 | CB | VAL B | 155 | 49.550 | 80.923 | −6.254 | 1.00 | 118.28 |
| 4305 | CG1 | VAL B | 155 | 50.030 | 79.574 | −6.748 | 1.00 | 118.28 |
| 4306 | CG2 | VAL B | 155 | 48.773 | 80.767 | −4.968 | 1.00 | 118.28 |
| 4307 | C | VAL B | 155 | 51.459 | 82.067 | −7.332 | 1.00 | 135.91 |
| 4308 | O | VAL B | 155 | 50.938 | 82.731 | −8.237 | 1.00 | 135.91 |
| 4309 | N | TRP B | 156 | 52.655 | 81.505 | −7.433 | 1.00 | 121.66 |
| 4310 | CA | TRP B | 156 | 53.453 | 81.702 | −8.624 | 1.00 | 121.66 |
| 4311 | CB | TRP B | 156 | 52.679 | 81.359 | −9.884 | 1.00 | 200.98 |
| 4312 | CG | TRP B | 156 | 52.385 | 79.963 | −9.953 | 1.00 | 200.98 |
| 4313 | CD2 | TRP B | 156 | 53.333 | 78.905 | −9.901 | 1.00 | 200.98 |
| 4314 | CE2 | TRP B | 156 | 52.609 | 77.699 | −9.939 | 1.00 | 200.98 |
| 4315 | CE3 | TRP B | 156 | 54.730 | 78.860 | −9.823 | 1.00 | 200.98 |
| 4316 | CD1 | TRP B | 156 | 51.160 | 79.393 | −10.027 | 1.00 | 200.98 |
| 4317 | NE1 | TRP B | 156 | 51.283 | 78.024 | −10.013 | 1.00 | 200.98 |
| 4318 | CZ2 | TRP B | 156 | 53.232 | 76.454 | −9.905 | 1.00 | 200.98 |
| 4319 | CZ3 | TRP B | 156 | 55.352 | 77.622 | −9.790 | 1.00 | 200.98 |
| 4320 | CH2 | TRP B | 156 | 54.599 | 76.432 | −9.834 | 1.00 | 200.98 |
| 4321 | C | TRP B | 156 | 53.739 | 83.168 | −8.671 | 1.00 | 121.66 |
| 4322 | O | TRP B | 156 | 54.677 | 83.647 | −8.029 | 1.00 | 121.66 |
| 4323 | N | GLN B | 157 | 52.883 | 83.878 | −9.403 | 1.00 | 111.84 |
| 4324 | CA | GLN B | 157 | 53.057 | 85.297 | −9.568 | 1.00 | 111.84 |
| 4325 | CB | GLN B | 157 | 53.912 | 85.525 | −10.808 | 1.00 | 249.48 |
| 4326 | CG | GLN B | 157 | 55.364 | 85.155 | −10.531 | 1.00 | 249.48 |
| 4327 | CD | GLN B | 157 | 55.818 | 85.806 | −9.260 | 1.00 | 249.48 |
| 4328 | OE1 | GLN B | 157 | 55.599 | 86.983 | −9.081 | 1.00 | 249.48 |
| 4329 | NE2 | GLN B | 157 | 56.437 | 85.047 | −8.360 | 1.00 | 249.48 |
| 4330 | C | GLN B | 157 | 51.781 | 86.092 | −9.614 | 1.00 | 111.84 |
| 4331 | O | GLN B | 157 | 51.785 | 87.265 | −9.988 | 1.00 | 111.84 |
| 4332 | N | LEU B | 158 | 50.688 | 85.447 | −9.217 | 1.00 | 140.68 |
| 4333 | CA | LEU B | 158 | 49.392 | 86.104 | −9.195 | 1.00 | 140.68 |
| 4334 | CB | LEU B | 158 | 48.463 | 85.512 | −10.253 | 1.00 | 225.85 |
| 4335 | CG | LEU B | 158 | 48.673 | 85.926 | −11.710 | 1.00 | 225.85 |
| 4336 | CD1 | LEU B | 158 | 47.296 | 86.093 | −12.325 | 1.00 | 225.85 |
| 4337 | CD2 | LEU B | 158 | 49.447 | 87.239 | −11.827 | 1.00 | 225.85 |
| 4338 | C | LEU B | 158 | 48.724 | 86.035 | −7.829 | 1.00 | 140.68 |
| 4339 | O | LEU B | 158 | 48.980 | 85.125 | −7.039 | 1.00 | 140.68 |
| 4340 | N | ASP B | 159 | 47.870 | 87.017 | −7.560 | 1.00 | 142.12 |
| 4341 | CA | ASP B | 159 | 47.162 | 87.101 | −6.291 | 1.00 | 142.12 |
| 4342 | CB | ASP B | 159 | 46.879 | 88.574 | −5.943 | 1.00 | 249.27 |
| 4343 | CG | ASP B | 159 | 48.138 | 89.443 | −5.949 | 1.00 | 249.27 |
| 4344 | OD1 | ASP B | 159 | 49.066 | 89.181 | −5.152 | 1.00 | 249.27 |
| 4345 | OD2 | ASP B | 159 | 48.194 | 90.399 | −6.754 | 1.00 | 249.27 |
| 4346 | C | ASP B | 159 | 45.846 | 86.325 | −6.363 | 1.00 | 142.12 |
| 4347 | O | ASP B | 159 | 45.204 | 86.280 | −7.414 | 1.00 | 142.12 |
| 4348 | N | TYR B | 160 | 45.456 | 85.711 | −5.244 | 1.00 | 173.61 |
| 4349 | CA | TYR B | 160 | 44.209 | 84.946 | −5.164 | 1.00 | 173.61 |
| 4350 | CB | TYR B | 160 | 44.442 | 83.463 | −5.430 | 1.00 | 249.32 |
| 4351 | CG | TYR B | 160 | 45.173 | 83.176 | −6.709 | 1.00 | 249.32 |
| 4352 | CD1 | TYR B | 160 | 46.561 | 83.098 | −6.731 | 1.00 | 249.32 |
| 4353 | CE1 | TYR B | 160 | 47.246 | 82.846 | −7.908 | 1.00 | 249.32 |
| 4354 | CD2 | TYR B | 160 | 44.481 | 82.995 | −7.903 | 1.00 | 249.32 |
| 4355 | CE2 | TYR B | 160 | 45.155 | 82.744 | −9.091 | 1.00 | 249.32 |
| 4356 | CZ | TYR B | 160 | 46.540 | 82.671 | −9.085 | 1.00 | 249.32 |
| 4357 | OH | TYR B | 160 | 47.221 | 82.428 | −10.254 | 1.00 | 249.32 |
| 4358 | C | TYR B | 160 | 43.517 | 85.072 | −3.818 | 1.00 | 173.61 |
| 4359 | O | TYR B | 160 | 44.155 | 85.185 | −2.768 | 1.00 | 173.61 |
| 4360 | N | GLU B | 161 | 42.194 | 85.014 | −3.871 | 1.00 | 90.61 |
| 4361 | CA | GLU B | 161 | 41.341 | 85.130 | −2.695 | 1.00 | 90.61 |
| 4362 | CB | GLU B | 161 | 40.333 | 86.257 | −2.926 | 1.00 | 219.32 |
| 4363 | CG | GLU B | 161 | 39.312 | 86.469 | −1.834 | 1.00 | 219.32 |
| 4364 | CD | GLU B | 161 | 38.626 | 87.808 | −1.980 | 1.00 | 219.32 |
| 4365 | OE1 | GLU B | 161 | 37.530 | 87.986 | −1.409 | 1.00 | 219.32 |
| 4366 | OE2 | GLU B | 161 | 39.197 | 88.688 | −2.663 | 1.00 | 219.32 |
| 4367 | C | GLU B | 161 | 40.634 | 83.792 | −2.513 | 1.00 | 90.61 |
| 4368 | O | GLU B | 161 | 40.215 | 83.179 | −3.486 | 1.00 | 90.61 |
| 4369 | N | SER B | 162 | 40.522 | 83.335 | −1.270 | 1.00 | 91.35 |
| 4370 | CA | SER B | 162 | 39.884 | 82.057 | −0.957 | 1.00 | 91.35 |
| 4371 | CB | SER B | 162 | 40.575 | 81.414 | 0.251 | 1.00 | 56.32 |
| 4372 | OG | SER B | 162 | 40.507 | 82.266 | 1.391 | 1.00 | 56.32 |
| 4373 | C | SER B | 162 | 38.401 | 82.220 | −0.664 | 1.00 | 91.35 |
| 4374 | O | SER B | 162 | 37.909 | 83.344 | −0.522 | 1.00 | 91.35 |
| 4375 | N | GLU B | 163 | 37.690 | 81.097 | −0.574 | 1.00 | 79.84 |
| 4376 | CA | GLU B | 163 | 36.253 | 81.114 | −0.287 | 1.00 | 79.84 |
| 4377 | CB | GLU B | 163 | 35.639 | 79.737 | −0.529 | 1.00 | 200.03 |
| 4378 | CG | GLU B | 163 | 35.475 | 79.360 | −1.993 | 1.00 | 200.03 |
| 4379 | CD | GLU B | 163 | 34.355 | 80.127 | −2.674 | 1.00 | 200.03 |
| 4380 | OE1 | GLU B | 163 | 33.206 | 80.054 | −2.187 | 1.00 | 200.03 |
| 4381 | OE2 | GLU B | 163 | 34.621 | 80.795 | −3.698 | 1.00 | 200.03 |
| 4382 | C | GLU B | 163 | 36.094 | 81.500 | 1.169 | 1.00 | 79.84 |
| 4383 | O | GLU B | 163 | 36.886 | 81.075 | 1.996 | 1.00 | 79.84 |
| 4384 | N | PRO B | 164 | 35.068 | 82.315 | 1.508 | 1.00 | 60.77 |
| 4385 | CD | PRO B | 164 | 34.003 | 82.898 | 0.682 | 1.00 | 73.97 |
| 4386 | CA | PRO B | 164 | 34.904 | 82.700 | 2.920 | 1.00 | 60.77 |
| 4387 | CB | PRO B | 164 | 33.877 | 83.829 | 2.847 | 1.00 | 73.97 |
| 4388 | CG | PRO B | 164 | 33.008 | 83.388 | 1.730 | 1.00 | 73.97 |
| 4389 | C | PRO B | 164 | 34.431 | 81.530 | 3.744 | 1.00 | 60.77 |
| 4390 | O | PRO B | 164 | 33.842 | 80.588 | 3.223 | 1.00 | 60.77 |
| 4391 | N | LEU B | 165 | 34.700 | 81.568 | 5.035 | 1.00 | 63.41 |
| 4392 | CA | LEU B | 165 | 34.288 | 80.472 | 5.889 | 1.00 | 63.41 |
| 4393 | CB | LEU B | 165 | 35.440 | 79.499 | 6.070 | 1.00 | 68.59 |
| 4394 | CG | LEU B | 165 | 35.185 | 78.450 | 7.138 | 1.00 | 68.59 |
| 4395 | CD1 | LEU B | 165 | 33.824 | 77.858 | 6.851 | 1.00 | 68.59 |
| 4396 | CD2 | LEU B | 165 | 36.264 | 77.380 | 7.140 | 1.00 | 68.59 |
| 4397 | C | LEU B | 165 | 33.847 | 80.963 | 7.250 | 1.00 | 63.41 |
| 4398 | O | LEU B | 165 | 34.613 | 81.635 | 7.953 | 1.00 | 63.41 |
| 4399 | N | ASN B | 166 | 32.623 | 80.613 | 7.628 | 1.00 | 64.97 |
| 4400 | CA | ASN B | 166 | 32.078 | 81.041 | 8.911 | 1.00 | 64.97 |
| 4401 | CB | ASN B | 166 | 30.556 | 81.011 | 8.900 | 1.00 | 96.05 |
| 4402 | CG | ASN B | 166 | 29.945 | 82.338 | 8.491 | 1.00 | 96.05 |
| 4403 | OD1 | ASN B | 166 | 30.504 | 83.403 | 8.737 | 1.00 | 96.05 |
| 4404 | ND2 | ASN B | 166 | 28.773 | 82.270 | 7.884 | 1.00 | 96.05 |
| 4405 | C | ASN B | 166 | 32.556 | 80.174 | 10.040 | 1.00 | 64.97 |
| 4406 | O | ASN B | 166 | 32.754 | 78.988 | 9.860 | 1.00 | 64.97 |
| 4407 | N | ILE B | 167 | 32.720 | 80.766 | 11.213 | 1.00 | 77.41 |
| 4408 | CA | ILE B | 167 | 33.183 | 80.034 | 12.375 | 1.00 | 77.41 |
| 4409 | CB | ILE B | 167 | 34.653 | 80.263 | 12.591 | 1.00 | 59.98 |
| 4410 | CG2 | ILE B | 167 | 35.050 | 79.859 | 13.985 | 1.00 | 59.98 |
| 4411 | CG1 | ILE B | 167 | 35.434 | 79.484 | 11.546 | 1.00 | 59.98 |
| 4412 | CD1 | ILE B | 167 | 36.942 | 79.537 | 11.784 | 1.00 | 59.98 |
| 4413 | C | ILE B | 167 | 32.467 | 80.488 | 13.622 | 1.00 | 77.41 |
| 4414 | O | ILE B | 167 | 32.375 | 81.676 | 13.896 | 1.00 | 77.41 |
| 4415 | N | THR B | 168 | 31.972 | 79.548 | 14.405 | 1.00 | 104.04 |
| 4416 | CA | THR B | 168 | 31.283 | 79.938 | 15.610 | 1.00 | 104.04 |
| 4417 | CB | THR B | 168 | 29.817 | 79.572 | 15.536 | 1.00 | 107.45 |
| 4418 | OG1 | THR B | 168 | 29.239 | 80.179 | 14.374 | 1.00 | 107.45 |
| 4419 | CG2 | THR B | 168 | 29.096 | 80.067 | 16.766 | 1.00 | 107.45 |
| 4420 | C | THR B | 168 | 31.888 | 79.326 | 16.850 | 1.00 | 104.04 |
| 4421 | O | THR B | 168 | 32.254 | 78.155 | 16.886 | 1.00 | 104.04 |
| 4422 | N | VAL B | 169 | 32.012 | 80.155 | 17.867 | 1.00 | 108.46 |
| 4423 | CA | VAL B | 169 | 32.544 | 79.737 | 19.146 | 1.00 | 108.46 |
| 4424 | CB | VAL B | 169 | 33.748 | 80.618 | 19.563 | 1.00 | 68.82 |
| 4425 | CG1 | VAL B | 169 | 33.974 | 80.539 | 21.049 | 1.00 | 68.82 |
| 4426 | CG2 | VAL B | 169 | 34.981 | 80.174 | 18.834 | 1.00 | 68.82 |
| 4427 | C | VAL B | 169 | 31.394 | 79.942 | 20.129 | 1.00 | 108.46 |
| 4428 | O | VAL B | 169 | 31.047 | 81.082 | 20.455 | 1.00 | 08.46 |
| 4429 | N | ILE B | 170 | 30.790 | 78.844 | 20.579 | 1.00 | 128.18 |
| 4430 | CA | ILE B | 170 | 29.679 | 78.917 | 21.525 | 1.00 | 128.18 |
| 4431 | CB | ILE B | 170 | 28.680 | 77.760 | 21.285 | 1.00 | 141.23 |
| 4432 | CG2 | ILE B | 170 | 28.276 | 77.749 | 19.833 | 1.00 | 141.23 |
| 4433 | CG1 | ILE B | 170 | 29.321 | 76.414 | 21.631 | 1.00 | 141.23 |
| 4434 | CD1 | ILE B | 170 | 28.423 | 75.218 | 21.463 | 1.00 | 141.23 |
| 4435 | C | ILE B | 170 | 30.228 | 78.854 | 22.946 | 1.00 | 128.18 |
| 4436 | O | ILE B | 170 | 31.426 | 78.679 | 23.142 | 1.00 | 128.18 |
| 4437 | N | LYS B | 171 | 29.365 | 79.005 | 23.941 | 1.00 | 164.76 |
| 4438 | CA | LYS B | 171 | 29.816 | 78.956 | 25.328 | 1.00 | 164.76 |
| 4439 | CB | LYS B | 171 | 29.779 | 80.358 | 25.929 | 1.00 | 211.84 |
| 4440 | CG | LYS B | 171 | 28.416 | 81.020 | 25.858 | 1.00 | 211.84 |
| 4441 | CD | LYS B | 171 | 28.536 | 82.537 | 25.911 | 1.00 | 211.84 |
| 4442 | CE | LYS B | 171 | 29.207 | 83.011 | 27.190 | 1.00 | 211.84 |
| 4443 | N2 | LYS B | 171 | 29.341 | 84.494 | 27.217 | 1.00 | 211.84 |
| 4444 | C | LYS B | 171 | 28.987 | 77.998 | 26.180 | 1.00 | 164.76 |
| 4445 | O | LYS B | 171 | 29.329 | 77.724 | 27.330 | 1.00 | 164.76 |
| 4446 | C1 | NAG B | 221 | 47.345 | 59.956 | −1.693 | 1.00 | 249.77 |
| 4447 | C2 | NAG B | 221 | 48.521 | 60.923 | −1.796 | 1.00 | 249.77 |
| 4448 | N2 | NAG B | 221 | 48.022 | 62.275 | −1.936 | 1.00 | 249.77 |
| 4449 | C7 | NAG B | 221 | 48.763 | 63.299 | −1.535 | 1.00 | 249.77 |
| 4450 | O7 | NAG B | 221 | 49.873 | 63.160 | −1.022 | 1.00 | 249.77 |
| 4451 | C8 | NAG B | 221 | 48.181 | 64.690 | −1.724 | 1.00 | 249.77 |
| 4452 | C3 | NAG B | 221 | 49.387 | 60.591 | −3.002 | 1.00 | 249.77 |
| 4453 | O3 | NAG B | 221 | 50.560 | 61.387 | −2.974 | 1.00 | 249.77 |
| 4454 | C4 | NAG B | 221 | 49.783 | 59.115 | −3.044 | 1.00 | 249.77 |
| 4455 | O4 | NAG B | 221 | 50.388 | 58.867 | −4.330 | 1.00 | 249.77 |
| 4456 | C5 | NAG B | 221 | 48.535 | 58.221 | −2.850 | 1.00 | 249.77 |
| 4457 | O5 | NAG B | 221 | 47.825 | 58.605 | −1.651 | 1.00 | 249.77 |
| 4458 | C6 | NAG B | 221 | 48.869 | 56.745 | −2.696 | 1.00 | 249.77 |
| 4459 | O6 | NAG B | 221 | 49.689 | 56.518 | −1.557 | 1.00 | 249.77 |
| 4460 | C1 | NAG B | 222 | 51.148 | 57.718 | −4.505 | 1.00 | 249.77 |
| 4461 | C2 | NAG B | 222 | 52.440 | 58.058 | −5.267 | 1.00 | 249.77 |
| 4462 | N2 | NAG B | 222 | 53.222 | 59.027 | −4.521 | 1.00 | 249.77 |
| 4463 | C7 | NAG B | 222 | 54.445 | 58.717 | −4.103 | 1.00 | 249.77 |
| 4464 | O7 | NAG B | 222 | 54.970 | 57.622 | −4.314 | 1.00 | 249.77 |
| 4465 | C8 | NAG B | 222 | 55.199 | 59.786 | −3.332 | 1.00 | 249.77 |
| 4466 | C3 | NAG B | 222 | 52.103 | 58.614 | −6.661 | 1.00 | 249.77 |
| 4467 | O3 | NAG B | 222 | 53.301 | 58.775 | −7.409 | 1.00 | 249.77 |
| 4468 | C4 | NAG B | 222 | 51.148 | 57.668 | −7.412 | 1.00 | 249.77 |
| 4469 | O4 | NAG B | 222 | 50.712 | 58.282 | −8.619 | 1.00 | 249.77 |
| 4470 | C5 | NAG B | 222 | 49.930 | 57.333 | −6.541 | 1.00 | 249.77 |
| 4471 | O5 | NAG B | 222 | 50.362 | 56.787 | −5.270 | 1.00 | 249.77 |
| 4472 | C6 | NAG B | 222 | 49.003 | 56.316 | −7.180 | 1.00 | 249.77 |
| 4473 | O6 | NAG B | 222 | 47.646 | 56.720 | −7.068 | 1.00 | 249.77 |
| 4474 | C1 | NAG B | 242 | 26.466 | 62.870 | −0.923 | 1.00 | 89.47 |
| 4475 | C2 | NAG B | 242 | 26.972 | 62.476 | −2.293 | 1.00 | 89.47 |
| 4476 | N2 | NAG B | 242 | 27.712 | 61.243 | −2.203 | 1.00 | 89.47 |
| 4477 | C7 | NAG B | 242 | 27.358 | 60.216 | −2.956 | 1.00 | 89.47 |
| 4478 | O7 | NAG B | 242 | 26.416 | 60.270 | −3.732 | 1.00 | 89.47 |
| 4479 | C8 | NAG B | 242 | 28.159 | 58.938 | −2.829 | 1.00 | 89.47 |
| 4480 | C3 | NAG B | 242 | 27.882 | 63.561 | −2.855 | 1.00 | 89.47 |
| 4481 | O3 | NAG B | 242 | 28.253 | 63.234 | −4.180 | 1.00 | 89.47 |
| 4482 | C4 | NAG B | 242 | 27.180 | 64.901 | −2.854 | 1.00 | 89.47 |
| 4483 | O4 | NAG B | 242 | 28.116 | 65.947 | −3.186 | 1.00 | 89.47 |
| 4484 | C5 | NAG B | 242 | 26.567 | 65.197 | −1.493 | 1.00 | 89.47 |
| 4485 | O5 | NAG B | 242 | 25.753 | 64.083 | −1.046 | 1.00 | 89.47 |
| 4486 | C6 | NAG B | 242 | 25.657 | 66.413 | −1.634 | 1.00 | 89.47 |
| 4487 | O6 | NAG B | 242 | 25.965 | 67.439 | −0.691 | 1.00 | 89.47 |
| 4488 | C1 | NAG B | 243 | 27.860 | 66.616 | −4.363 | 1.00 | 124.06 |
| 4489 | C2 | NAG B | 243 | 28.444 | 68.031 | −4.311 | 1.00 | 124.06 |
| 4490 | N2 | NAG B | 243 | 27.812 | 68.814 | −3.263 | 1.00 | 124.06 |
| 4491 | C7 | NAG B | 243 | 28.560 | 69.543 | −2.441 | 1.00 | 124.06 |
| 4492 | O7 | NAG B | 243 | 29.786 | 69.568 | −2.502 | 1.00 | 124.06 |
| 4493 | C8 | NAG B | 243 | 27.853 | 70.353 | −1.378 | 1.00 | 124.06 |
| 4494 | C3 | NAG B | 243 | 28.214 | 68.724 | −5.658 | 1.00 | 124.06 |
| 4495 | O3 | NAG B | 243 | 28.825 | 70.012 | −5.653 | 1.00 | 124.06 |
| 4496 | C4 | NAG B | 243 | 28.765 | 67.860 | −6.816 | 1.00 | 124.06 |
| 4497 | O4 | NAG B | 243 | 28.392 | 68.459 | −8.089 | 1.00 | 124.06 |
| 4498 | C5 | NAG B | 243 | 28.162 | 66.455 | −6.717 | 1.00 | 124.06 |
| 4499 | O5 | NAG B | 243 | 28.449 | 65.870 | −5.432 | 1.00 | 124.06 |
| 4500 | C6 | NAG B | 243 | 28.638 | 65.499 | −7.762 | 1.00 | 124.06 |
| 4501 | O6 | NAG B | 243 | 30.003 | 65.214 | −7.571 | 1.00 | 124.06 |
| 4502 | C1 | MAN B | 244 | 29.308 | 68.650 | −9.080 | 1.00 | 182.20 |
| 4503 | C2 | MAN B | 244 | 30.527 | 69.553 | −8.800 | 1.00 | 182.20 |
| 4504 | O2 | MAN B | 244 | 31.636 | 68.751 | −8.489 | 1.00 | 182.20 |
| 4505 | C3 | MAN B | 244 | 30.736 | 70.260 | −10.177 | 1.00 | 182.20 |
| 4506 | O3 | MAN B | 244 | 31.834 | 71.153 | −10.165 | 1.00 | 182.20 |
| 4507 | C4 | MAN B | 244 | 30.850 | 69.264 | −11.367 | 1.00 | 182.20 |
| 4508 | O4 | MAN B | 244 | 31.059 | 69.973 | −12.588 | 1.00 | 182.20 |
| 4509 | C5 | MAN B | 244 | 29.519 | 68.480 | −11.433 | 1.00 | 182.20 |
| 4510 | O5 | MAN B | 244 | 29.290 | 67.732 | −10.210 | 1.00 | 182.20 |
| 4511 | C6 | MAN B | 244 | 29.376 | 67.561 | −12.650 | 1.00 | 182.20 |
| 4512 | O6 | MAN B | 244 | 30.030 | 66.327 | −12.454 | 1.00 | 182.20 |
| 4513 | C1 | NAG B | 250 | 42.367 | 49.115 | 8.367 | 1.00 | 249.70 |
| 4514 | C2 | NAG B | 250 | 43.729 | 49.074 | 9.087 | 1.00 | 249.70 |
| 4515 | N2 | NAG B | 250 | 43.544 | 49.049 | 10.526 | 1.00 | 249.70 |
| 4516 | C7 | NAG B | 250 | 43.853 | 47.960 | 11.227 | 1.00 | 249.70 |
| 4517 | O7 | NAG B | 250 | 44.295 | 46.930 | 10.709 | 1.00 | 249.70 |
| 4518 | C8 | NAG B | 250 | 43.632 | 48.021 | 12.734 | 1.00 | 249.70 |
| 4519 | C3 | NAG B | 250 | 44.545 | 50.311 | 8.692 | 1.00 | 249.70 |
| 4520 | O3 | NAG B | 250 | 45.842 | 50.245 | 9.269 | 1.00 | 249.70 |
| 4521 | C4 | NAG B | 250 | 44.660 | 50.407 | 7.167 | 1.00 | 249.70 |
| 4522 | O4 | NAG B | 250 | 45.304 | 51.625 | 6.813 | 1.00 | 249.70 |
| 4523 | C5 | NAG B | 250 | 43.262 | 50.349 | 6.521 | 1.00 | 249.70 |
| 4524 | O5 | NAG B | 250 | 42.562 | 49.158 | 6.946 | 1.00 | 249.70 |
| 4525 | C6 | NAG B | 250 | 43.315 | 50.314 | 5.003 | 1.00 | 249.70 |
| 4526 | O6 | NAG B | 250 | 42.060 | 49.940 | 4.449 | 1.00 | 249.70 |
| 4527 | C1 | NAG B | 274 | 20.954 | 54.260 | 22.053 | 1.00 | 246.89 |
| 4528 | C2 | NAG B | 274 | 20.822 | 55.380 | 23.099 | 1.00 | 246.89 |
| 4529 | N2 | NAG 8 | 274 | 21.918 | 55.314 | 24.050 | 1.00 | 246.89 |
| 4530 | C7 | NAG B | 274 | 22.298 | 56.407 | 24.706 | 1.00 | 246.89 |
| 4531 | O7 | NAG B | 274 | 21.767 | 57.506 | 24.541 | 1.00 | 246.89 |
| 4532 | C8 | NAG B | 274 | 23.446 | 56.263 | 25.690 | 1.00 | 246.89 |
| 4533 | C3 | NAG B | 274 | 19.484 | 55.246 | 23.844 | 1.00 | 246.89 |
| 4534 | O3 | NAG B | 274 | 19.302 | 56.360 | 24.707 | 1.00 | 246.89 |
| 4535 | C4 | NAG B | 274 | 18.314 | 55.163 | 22.856 | 1.00 | 246.89 |
| 4536 | O4 | NAG B | 274 | 17.111 | 54.887 | 23.563 | 1.00 | 246.89 |
| 4537 | C5 | NAG B | 274 | 18.576 | 54.059 | 21.820 | 1.00 | 246.89 |
| 4538 | O5 | NAG B | 274 | 19.837 | 54.291 | 21.151 | 1.00 | 246.89 |
| 4539 | C6 | NAG B | 274 | 17.507 | 53.987 | 20.743 | 1.00 | 246.89 |
| 4540 | O6 | NAG B | 274 | 17.896 | 53.120 | 19.688 | 1.00 | 246.89 |
| 4541 | C1 | NAG B | 335 | 50.085 | 74.386 | 8.041 | 1.00 | 247.49 |
| 4542 | C2 | NAG B | 335 | 50.430 | 73.230 | 9.006 | 1.00 | 247.49 |
| 4543 | N2 | NAG B | 335 | 50.451 | 73.760 | 10.357 | 1.00 | 247.49 |
| 4544 | C7 | NAG B | 335 | 49.583 | 73.330 | 11.267 | 1.00 | 247.49 |
| 4545 | O7 | NAG B | 335 | 48.734 | 72.471 | 11.036 | 1.00 | 247.49 |
| 4546 | C8 | NAG B | 335 | 49.680 | 73.947 | 12.649 | 1.00 | 247.49 |
| 4547 | C3 | NAG B | 335 | 51.781 | 72.552 | 8.725 | 1.00 | 247.49 |
| 4548 | O3 | NAG B | 335 | 51.808 | 71.282 | 9.359 | 1.00 | 247.49 |
| 4549 | C4 | NAG B | 335 | 52.016 | 72.369 | 7.231 | 1.00 | 247.49 |
| 4550 | O4 | NAG B | 335 | 53.304 | 71.813 | 7.004 | 1.00 | 247.49 |
| 4551 | C5 | NAG B | 335 | 51.906 | 73.729 | 6.561 | 1.00 | 247.49 |
| 4552 | O5 | NAG B | 335 | 50.550 | 74.212 | 6.679 | 1.00 | 247.49 |
| 4553 | C6 | NAG B | 335 | 52.229 | 73.654 | 5.078 | 1.00 | 247.49 |
| 4554 | O6 | NAG B | 335 | 53.343 | 74.471 | 4.748 | 1.00 | 247.49 |
| 4555 | C1 | NAG B | 340 | 41.414 | 81.009 | 28.648 | 1.00 | 249.67 |
| 4556 | C2 | NAG B | 340 | 40.114 | 80.981 | 29.434 | 1.00 | 249.67 |
| 4557 | N2 | NAG B | 340 | 38.971 | 81.033 | 28.539 | 1.00 | 249.67 |
| 4558 | C7 | NAG B | 340 | 37.997 | 80.133 | 28.666 | 1.00 | 249.67 |
| 4559 | O7 | NAG B | 340 | 38.012 | 79.245 | 29.526 | 1.00 | 249.67 |
| 4560 | C8 | NAG B | 340 | 36.831 | 80.226 | 27.702 | 1.00 | 249.67 |
| 4561 | C3 | NAG B | 340 | 40.092 | 82.143 | 30.420 | 1.00 | 249.67 |
| 4562 | O3 | NAG B | 340 | 38.904 | 82.071 | 31.207 | 1.00 | 249.67 |
| 4563 | C4 | NAG B | 340 | 41.329 | 82.067 | 31.330 | 1.00 | 249.67 |
| 4564 | O4 | NAG B | 340 | 41.393 | 83.255 | 32.105 | 1.00 | 249.67 |
| 4565 | C5 | NAG B | 340 | 42.643 | 81.894 | 30.520 | 1.00 | 249.67 |
| 4566 | O5 | NAG B | 340 | 42.519 | 80.841 | 29.539 | 1.00 | 249.67 |
| 4567 | C6 | NAG B | 340 | 43.832 | 81.515 | 31.388 | 1.00 | 249.67 |
| 4568 | O6 | NAG B | 340 | 44.745 | 80.677 | 30.696 | 1.00 | 249.67 |
| 4569 | C1 | NAG B | 366 | 28.147 | 83.475 | 7.400 | 1.00 | 133.05 |
| 4570 | C2 | NAG B | 366 | 27.352 | 83.132 | 6.154 | 1.00 | 133.05 |
| 4571 | N2 | NAG B | 366 | 28.247 | 82.591 | 5.149 | 1.00 | 133.05 |
| 4572 | C7 | NAG B | 366 | 28.452 | 81.278 | 5.075 | 1.00 | 133.05 |
| 4573 | O7 | NAG B | 366 | 27.909 | 80.467 | 5.829 | 1.00 | 133.05 |
| 4574 | C8 | NAG B | 366 | 29.408 | 80.789 | 3.998 | 1.00 | 133.05 |
| 4575 | C3 | NAG B | 366 | 26.651 | 84.373 | 5.618 | 1.00 | 133.05 |
| 4576 | O3 | NAG B | 366 | 25.783 | 84.003 | 4.553 | 1.00 | 133.05 |
| 4577 | C4 | NAG B | 366 | 25.842 | 85.068 | 6.713 | 1.00 | 133.05 |
| 4578 | O4 | NAG B | 366 | 25.403 | 86.347 | 6.211 | 1.00 | 133.05 |
| 4579 | C5 | NAG B | 366 | 26.688 | 85.270 | 7.986 | 1.00 | 133.05 |
| 4580 | O5 | NAG B | 366 | 27.291 | 84.029 | 8.400 | 1.00 | 133.05 |
| 4581 | C6 | NAG B | 366 | 25.864 | 85.757 | 9.163 | 1.00 | 133.05 |
| 4582 | O6 | NAG B | 366 | 26.677 | 85.957 | 10.310 | 1.00 | 133.05 |
| 4583 | C1 | NAG B | 367 | 24.042 | 86.610 | 6.284 | 1.00 | 230.72 |
| 4584 | C2 | NAG B | 367 | 23.806 | 88.121 | 6.264 | 1.00 | 230.72 |
| 4585 | N2 | NAG B | 367 | 24.497 | 88.757 | 7.369 | 1.00 | 230.72 |
| 4586 | C7 | NAG B | 367 | 25.574 | 89.501 | 7.133 | 1.00 | 230.72 |
| 4587 | O7 | NAG B | 367 | 26.030 | 89.681 | 6.002 | 1.00 | 230.72 |
| 4588 | C8 | NAG B | 367 | 26.251 | 90.141 | 8.334 | 1.00 | 230.72 |
| 4589 | C3 | NAG B | 367 | 22.301 | 88.392 | 6.337 | 1.00 | 230.72 |
| 4590 | O3 | NAG B | 367 | 22.054 | 89.791 | 6.274 | 1.00 | 230.72 |
| 4591 | C4 | NAG B | 367 | 21.604 | 87.688 | 5.169 | 1.00 | 230.72 |
| 4592 | O4 | NAG B | 367 | 20.197 | 87.854 | 5.276 | 1.00 | 230.72 |
| 4593 | C5 | NAG B | 367 | 21.956 | 86.193 | 5.170 | 1.00 | 230.72 |
| 4594 | O5 | NAG B | 367 | 23.395 | 86.007 | 5.152 | 1.00 | 230.72 |
| 4595 | C6 | NAG B | 367 | 21.396 | 85.477 | 3.959 | 1.00 | 230.72 |
| 4596 | O6 | NAG B | 367 | 22.431 | 85.064 | 3.078 | 1.00 | 230.72 |
| 4597 | CB | LYS D | 4 | 55.111 | 67.727 | 55.236 | 1.00 | 220.56 |
| 4598 | CG | LYS D | 4 | 54.671 | 66.297 | 54.972 | 1.00 | 220.56 |
| 4599 | CD | LYS D | 4 | 54.274 | 65.601 | 56.262 | 1.00 | 220.56 |
| 4600 | CE | LYS D | 4 | 53.817 | 64.172 | 56.007 | 1.00 | 220.56 |
| 4601 | NZ | LYS D | 4 | 53.427 | 63.496 | 57.274 | 1.00 | 220.56 |
| 4602 | C | LYS D | 4 | 54.245 | 68.471 | 53.028 | 1.00 | 175.22 |
| 4603 | O | LYS D | 4 | 53.112 | 68.293 | 53.459 | 1.00 | 175.22 |
| 4604 | N | LYS D | 4 | 55.813 | 69.908 | 54.320 | 1.00 | 175.22 |
| 4605 | CA | LYS D | 4 | 55.442 | 68.509 | 53.968 | 1.00 | 175.22 |
| 4606 | N | PRO D | 5 | 54.485 | 68.654 | 51.722 | 1.00 | 119.81 |
| 4607 | CD | PRO D | 5 | 55.727 | 69.166 | 51.125 | 1.00 | 90.67 |
| 4608 | CA | PRO D | 5 | 53.397 | 68.631 | 50.737 | 1.00 | 119.81 |
| 4609 | CB | PRO D | 5 | 53.950 | 69.490 | 49.602 | 1.00 | 90.67 |
| 4610 | CG | PRO D | 5 | 55.400 | 69.182 | 49.643 | 1.00 | 90.67 |
| 4611 | C | PRO D | 5 | 53.035 | 67.215 | 50.281 | 1.00 | 119.81 |
| 4612 | O | PRO D | 5 | 53.836 | 66.281 | 50.412 | 1.00 | 119.81 |
| 4613 | N | LYS D | 6 | 51.824 | 67.054 | 49.752 | 1.00 | 96.52 |
| 4614 | CA | LYS D | 6 | 51.373 | 65.747 | 49.285 | 1.00 | 96.52 |
| 4615 | CB | LYS D | 6 | 50.549 | 65.060 | 50.379 | 1.00 | 171.50 |
| 4616 | CG | LYS D | 6 | 50.141 | 63.639 | 50.041 | 1.00 | 171.50 |
| 4617 | CD | LYS D | 6 | 49.490 | 62.929 | 51.225 | 1.00 | 171.50 |
| 4618 | CE | LYS D | 6 | 49.128 | 61.483 | 50.860 | 1.00 | 171.50 |
| 4619 | NZ | LYS D | 6 | 48.560 | 60.707 | 52.003 | 1.00 | 171.50 |
| 4620 | C | LYS D | 6 | 50.557 | 65.881 | 47.994 | 1.00 | 96.52 |
| 4621 | O | LYS D | 6 | 49.495 | 66.491 | 47.981 | 1.00 | 96.52 |
| 4622 | N | VAL D | 7 | 51.072 | 65.306 | 46.911 | 1.00 | 68.94 |
| 4623 | CA | VAL D | 7 | 50.422 | 65.353 | 45.604 | 1.00 | 68.94 |
| 4624 | CB | VAL D | 7 | 51.321 | 64.793 | 44.498 | 1.00 | 87.54 |
| 4625 | CG1 | VAL D | 7 | 50.661 | 65.026 | 43.147 | 1.00 | 87.54 |
| 4626 | CG2 | VAL D | 7 | 52.693 | 65.408 | 44.566 | 1.00 | 87.54 |
| 4627 | C | VAL D | 7 | 49.159 | 64.529 | 45.521 | 1.00 | 68.94 |
| 4628 | O | VAL D | 7 | 49.213 | 63.311 | 45.658 | 1.00 | 68.94 |
| 4629 | N | SER D | 8 | 48.033 | 65.178 | 45.263 | 1.00 | 67.77 |
| 4630 | CA | SER D | 8 | 46.766 | 64.465 | 45.138 | 1.00 | 67.77 |
| 4631 | CB | SER D | 8 | 45.651 | 65.209 | 45.877 | 1.00 | 176.15 |
| 4632 | OG | SER D | 8 | 45.554 | 66.551 | 45.438 | 1.00 | 176.15 |
| 4633 | C | SER D | 8 | 46.434 | 64.349 | 43.651 | 1.00 | 67.77 |
| 4634 | O | SER D | 8 | 47.041 | 65.043 | 42.834 | 1.00 | 67.77 |
| 4635 | N | LEU D | 9 | 45.500 | 63.459 | 43.304 | 1.00 | 116.14 |
| 4636 | CA | LEU D | 9 | 45.098 | 63.252 | 41.912 | 1.00 | 116.14 |
| 4637 | CB | LEU D | 9 | 45.531 | 61.883 | 41.396 | 1.00 | 98.23 |
| 4638 | CG | LEU D | 9 | 47.001 | 61.491 | 41.352 | 1.00 | 98.23 |
| 4639 | CD1 | LEU D | 9 | 47.193 | 60.359 | 40.372 | 1.00 | 98.23 |
| 4640 | CD2 | LEU D | 9 | 47.818 | 62.671 | 40.913 | 1.00 | 98.23 |
| 4641 | C | LEU D | 9 | 43.596 | 63.326 | 41.770 | 1.00 | 116.14 |
| 4642 | O | LEU D | 9 | 42.865 | 63.094 | 42.732 | 1.00 | 116.14 |
| 4643 | N | ASN D | 10 | 43.135 | 63.630 | 40.560 | 1.00 | 87.18 |
| 4644 | CA | ASN D | 10 | 41.699 | 63.718 | 40.284 | 1.00 | 87.18 |
| 4645 | CB | ASN D | 10 | 41.130 | 65.052 | 40.768 | 1.00 | 123.83 |
| 4646 | CG | ASN D | 10 | 39.625 | 65.064 | 40.746 | 1.00 | 123.83 |
| 4647 | OD1 | ASN D | 10 | 38.973 | 64.342 | 41.505 | 1.00 | 123.83 |
| 4648 | ND2 | ASN D | 10 | 39.058 | 65.867 | 39.857 | 1.00 | 123.83 |
| 4649 | C | ASN D | 10 | 41.419 | 63.561 | 38.797 | 1.00 | 87.18 |
| 4650 | O | ASN D | 10 | 41.732 | 64.453 | 38.000 | 1.00 | 87.18 |
| 4651 | N | PRO D | 11 | 40.804 | 62.432 | 38.402 | 1.00 | 137.25 |
| 4652 | CD | PRO D | 11 | 40.609 | 62.151 | 36.972 | 1.00 | 119.64 |
| 4653 | CA | PRO D | 11 | 40.349 | 61.301 | 39.221 | 1.00 | 137.25 |
| 4654 | CB | PRO D | 11 | 39.877 | 60.298 | 38.167 | 1.00 | 119.64 |
| 4655 | CG | PRO D | 11 | 39.503 | 61.164 | 37.007 | 1.00 | 119.64 |
| 4656 | C | PRO D | 11 | 41.422 | 60.689 | 40.148 | 1.00 | 137.25 |
| 4657 | O | PRO D | 11 | 42.614 | 60.926 | 39.952 | 1.00 | 137.25 |
| 4658 | N | PRO D | 12 | 41.017 | 59.899 | 41.164 | 1.00 | 96.57 |
| 4659 | CD | PRO D | 12 | 39.630 | 59.557 | 41.534 | 1.00 | 83.91 |
| 4660 | CA | PRO D | 12 | 41.951 | 59.269 | 42.104 | 1.00 | 96.57 |
| 4661 | CB | PRO D | 12 | 41.041 | 58.629 | 43.151 | 1.00 | 83.91 |
| 4662 | CG | PRO D | 12 | 39.761 | 59.344 | 43.011 | 1.00 | 83.91 |
| 4663 | C | PRO D | 12 | 42.774 | 58.205 | 41.374 | 1.00 | 96.57 |
| 4664 | O | PRO D | 12 | 43.874 | 57.834 | 41.802 | 1.00 | 96.57 |
| 4665 | N | TRP D | 13 | 42.222 | 57.717 | 40.268 | 1.00 | 86.01 |
| 4666 | CA | TRP D | 13 | 42.869 | 56.675 | 39.486 | 1.00 | 86.01 |
| 4667 | CB | TRP D | 13 | 42.032 | 56.366 | 38.247 | 1.00 | 97.24 |
| 4668 | CG | TRP D | 13 | 40.601 | 56.191 | 38.568 | 1.00 | 97.24 |
| 4669 | CD2 | TRP D | 13 | 40.049 | 55.556 | 39.724 | 1.00 | 97.24 |
| 4670 | CE2 | TRP D | 13 | 38.651 | 55.664 | 39.631 | 1.00 | 97.24 |
| 4671 | CE3 | TRP D | 13 | 40.604 | 54.902 | 40.836 | 1.00 | 97.24 |
| 4672 | CD1 | TRP D | 13 | 39.546 | 56.637 | 37.836 | 1.00 | 97.24 |
| 4673 | NE1 | TRP D | 13 | 38.370 | 56.330 | 38.469 | 1.00 | 97.24 |
| 4674 | CZ2 | TRP D | 13 | 37.795 | 55.147 | 40.609 | 1.00 | 97.24 |
| 4675 | CZ3 | TRP D | 13 | 39.753 | 54.387 | 41.804 | 1.00 | 97.24 |
| 4676 | CH2 | TRP D | 13 | 33.364 | 54.515 | 41.686 | 1.00 | 97.24 |
| 4677 | C | TRP D | 13 | 44.278 | 57.041 | 39.075 | 1.00 | 86.01 |
| 4678 | O | TRP D | 13 | 44.493 | 58.036 | 38.401 | 1.00 | 86.01 |
| 4679 | N | ASN D | 14 | 45.244 | 56.231 | 39.488 | 1.00 | 79.52 |
| 4680 | CA | ASN D | 14 | 46.627 | 56.488 | 39.122 | 1.00 | 79.52 |
| 4681 | CB | ASN D | 14 | 47.534 | 56.449 | 40.358 | 1.00 | 103.31 |
| 4682 | CG | ASN D | 14 | 47.664 | 55.067 | 40.958 | 1.00 | 103.31 |
| 4683 | OD1 | ASN D | 14 | 46.671 | 54.415 | 41.283 | 1.00 | 103.31 |
| 4684 | ND2 | ASN D | 14 | 48.895 | 54.618 | 41.124 | 1.00 | 103.31 |
| 4685 | C | ASN D | 14 | 47.153 | 55.545 | 38.031 | 1.00 | 79.52 |
| 4686 | O | ASN D | 14 | 48.358 | 55.444 | 37.825 | 1.00 | 79.52 |
| 4687 | N | ARG D | 15 | 46.248 | 54.842 | 37.351 | 1.00 | 58.96 |
| 4688 | CA | ARG D | 15 | 46.609 | 53.977 | 36.231 | 1.00 | 58.96 |
| 4689 | CB | ARG D | 15 | 46.413 | 52.517 | 36.552 | 1.00 | 70.76 |
| 4690 | CG | ARG D | 15 | 46.829 | 52.131 | 37.918 | 1.00 | 70.76 |
| 4691 | CD | ARG D | 15 | 46.633 | 50.641 | 38.077 | 1.00 | 70.76 |
| 4692 | NE | ARG D | 15 | 47.557 | 49.869 | 37.263 | 1.00 | 70.76 |
| 4693 | CZ | ARG D | 15 | 47.280 | 48.660 | 36.802 | 1.00 | 70.76 |
| 4694 | NH1 | ARG D | 15 | 46.108 | 48.121 | 37.078 | 1.00 | 70.76 |
| 4695 | NH2 | ARG D | 15 | 48.170 | 47.981 | 36.079 | 1.00 | 70.76 |
| 4696 | C | ARG D | 15 | 45.573 | 54.375 | 35.202 | 1.00 | 58.96 |
| 4697 | O | ARG D | 15 | 44.384 | 54.102 | 35.367 | 1.00 | 58.96 |
| 4698 | N | ILE D | 16 | 46.006 | 55.037 | 34.144 | 1.00 | 65.25 |
| 4699 | CA | ILE D | 16 | 45.052 | 55.457 | 33.146 | 1.00 | 65.25 |
| 4700 | CB | ILE D | 16 | 44.923 | 56.967 | 33.117 | 1.00 | 107.28 |
| 4701 | CG2 | ILE D | 16 | 44.319 | 57.455 | 34.414 | 1.00 | 107.28 |
| 4702 | CG1 | ILE D | 16 | 46.303 | 57.587 | 32.876 | 1.00 | 107.28 |
| 4703 | CD1 | ILE D | 16 | 46.295 | 59.099 | 32.854 | 1.00 | 107.28 |
| 4704 | C | ILE D | 16 | 45.380 | 54.992 | 31.754 | 1.00 | 65.25 |
| 4705 | O | ILE D | 16 | 46.492 | 54.553 | 31.461 | 1.00 | 65.25 |
| 4706 | N | PHE D | 17 | 44.373 | 55.117 | 30.905 | 1.00 | 82.89 |
| 4707 | CA | PHE D | 17 | 44.429 | 54.750 | 29.509 | 1.00 | 82.89 |
| 4708 | CB | PHE D | 17 | 43.011 | 54.508 | 29.030 | 1.00 | 73.74 |
| 4709 | CG | PHE D | 17 | 42.550 | 53.099 | 29.186 | 1.00 | 73.74 |
| 4710 | CD1 | PHE D | 17 | 41.245 | 52.822 | 29.578 | 1.00 | 73.74 |
| 4711 | CD2 | PHE D | 17 | 43.379 | 52.052 | 28.825 | 1.00 | 73.74 |
| 4712 | CE1 | PHE D | 17 | 40.779 | 51.529 | 29.625 | 1.00 | 73.74 |
| 4713 | CE2 | PHE D | 17 | 42.918 | 50.741 | 28.866 | 1.00 | 73.74 |
| 4714 | CZ | PHE D | 17 | 41.609 | 50.484 | 29.258 | 1.00 | 73.74 |
| 4715 | C | PHE D | 17 | 45.066 | 55.863 | 28.677 | 1.00 | 82.89 |
| 4716 | O | PHE D | 17 | 45.154 | 57.009 | 29.117 | 1.00 | 82.89 |
| 4717 | N | LYS D | 18 | 45.502 | 55.531 | 27.469 | 1.00 | 90.77 |
| 4718 | CA | LYS D | 18 | 46.117 | 56.516 | 26.588 | 1.00 | 90.77 |
| 4719 | CB | LYS D | 18 | 46.681 | 55.810 | 25.357 | 1.00 | 139.85 |
| 4720 | CG | LYS D | 18 | 47.467 | 56.691 | 24.410 | 1.00 | 139.85 |
| 4721 | CD | LYS D | 18 | 48.254 | 55.822 | 23.441 | 1.00 | 139.85 |
| 4722 | CE | LYS D | 18 | 49.094 | 56.637 | 22.472 | 1.00 | 139.85 |
| 4723 | NZ | LYS D | 18 | 48.256 | 57.533 | 21.630 | 1.00 | 139.85 |
| 4724 | C | LYS D | 18 | 45.079 | 57.556 | 26.156 | 1.00 | 90.77 |
| 4725 | O | LYS D | 18 | 43.975 | 57.212 | 25.731 | 1.00 | 90.77 |
| 4726 | N | GLY D | 19 | 45.420 | 58.832 | 26.284 | 1.00 | 135.30 |
| 4727 | CA | GLY D | 19 | 44.501 | 59.869 | 25.859 | 1.00 | 135.30 |
| 4728 | C | GLY D | 19 | 43.585 | 60.458 | 26.909 | 1.00 | 135.30 |
| 4729 | O | GLY D | 19 | 42.914 | 61.451 | 26.641 | 1.00 | 135.30 |
| 4730 | N | GLU D | 20 | 43.539 | 59.863 | 28.096 | 1.00 | 90.73 |
| 4731 | CA | GLU D | 20 | 42.679 | 60.387 | 29.158 | 1.00 | 90.73 |
| 4732 | CB | GLU D | 20 | 42.370 | 59.283 | 30.165 | 1.00 | 145.66 |
| 4733 | CG | GLU D | 20 | 41.858 | 58.007 | 29.497 | 1.00 | 145.66 |
| 4734 | CD | GLU D | 20 | 41.421 | 56.945 | 30.491 | 1.00 | 145.66 |
| 4735 | OE1 | GLU D | 20 | 42.233 | 56.567 | 31.363 | 1.00 | 145.66 |
| 4736 | OE2 | GLU D | 20 | 40.265 | 56.483 | 30.393 | 1.00 | 145.66 |
| 4737 | C | GLU D | 20 | 43.339 | 61.593 | 29.844 | 1.00 | 90.73 |
| 4738 | O | GLU D | 20 | 44.510 | 61.887 | 29.590 | 1.00 | 90.73 |
| 4739 | N | ASN D | 21 | 42.592 | 62.311 | 30.682 | 1.00 | 106.51 |
| 4740 | CA | ASN D | 21 | 43.163 | 63.469 | 31.364 | 1.00 | 106.51 |
| 4741 | CB | ASN D | 21 | 42.409 | 64.761 | 31.030 | 1.00 | 191.80 |
| 4742 | CG | ASN D | 21 | 42.014 | 64.865 | 29.580 | 1.00 | 191.80 |
| 4743 | OD1 | ASN D | 21 | 42.781 | 64.543 | 28.677 | 1.00 | 191.80 |
| 4744 | ND2 | ASN D | 21 | 40.797 | 65.346 | 29.365 | 1.00 | 191.80 |
| 4745 | C | ASN D | 21 | 43.127 | 63.300 | 32.872 | 1.00 | 106.51 |
| 4746 | O | ASN D | 21 | 42.165 | 62.756 | 33.424 | 1.00 | 106.51 |
| 4747 | N | VAL D | 22 | 44.170 | 63.792 | 33.533 | 1.00 | 83.07 |
| 4748 | CA | VAL D | 22 | 44.267 | 63.727 | 34.984 | 1.00 | 83.07 |
| 4749 | CB | VAL D | 22 | 45.143 | 62.571 | 35.425 | 1.00 | 85.11 |
| 4750 | CG1 | VAL D | 22 | 46.555 | 62.774 | 34.923 | 1.00 | 85.11 |
| 4751 | CG2 | VAL D | 22 | 45.134 | 62.475 | 36.937 | 1.00 | 85.11 |
| 4752 | C | VAL D | 22 | 44.885 | 65.015 | 35.514 | 1.00 | 83.07 |
| 4753 | O | VAL D | 22 | 45.701 | 65.643 | 34.833 | 1.00 | 83.07 |
| 4754 | N | THR D | 23 | 44.517 | 65.401 | 36.731 | 1.00 | 66.58 |
| 4755 | CA | THR D | 23 | 45.024 | 66.635 | 37.318 | 1.00 | 66.58 |
| 4756 | CB | THR D | 23 | 43.848 | 67.553 | 37.646 | 1.00 | 160.89 |
| 4757 | OG1 | THR D | 23 | 43.036 | 67.717 | 36.477 | 1.00 | 160.89 |
| 4758 | CG2 | THR D | 23 | 44.344 | 68.902 | 38.121 | 1.00 | 160.89 |
| 4759 | C | THR D | 23 | 45.820 | 66.391 | 38.598 | 1.00 | 66.58 |
| 4760 | O | THR D | 23 | 45.330 | 65.713 | 39.498 | 1.00 | 66.58 |
| 4761 | N | LEU D | 24 | 47.035 | 66.923 | 38.705 | 1.00 | 91.98 |
| 4762 | CA | LEU D | 24 | 47.810 | 66.708 | 39.933 | 1.00 | 91.98 |
| 4763 | CB | LEU D | 24 | 49.235 | 66.263 | 39.632 | 1.00 | 82.54 |
| 4764 | CG | LEU D | 24 | 49.491 | 65.315 | 38.471 | 1.00 | 82.54 |
| 4765 | CD1 | LEU D | 24 | 50.891 | 64.750 | 38.587 | 1.00 | 82.54 |
| 4766 | CD2 | LEU D | 24 | 48.509 | 64.210 | 38.476 | 1.00 | 82.54 |
| 4767 | C | LEU D | 24 | 47.882 | 67.967 | 40.785 | 1.00 | 91.98 |
| 4768 | O | LEU D | 24 | 48.622 | 68.895 | 40.479 | 1.00 | 91.98 |
| 4769 | N | THR D | 25 | 47.131 | 67.992 | 41.873 | 1.00 | 89.48 |
| 4770 | CA | THR D | 25 | 47.122 | 69.153 | 42.732 | 1.00 | 89.48 |
| 4771 | CB | THR D | 25 | 45.754 | 69.300 | 43.385 | 1.00 | 145.87 |
| 4772 | OG1 | THR D | 25 | 44.757 | 69.342 | 42.357 | 1.00 | 145.87 |
| 4773 | CG2 | THR D | 25 | 45.686 | 70.568 | 44.198 | 1.00 | 145.87 |
| 4774 | C | THR D | 25 | 48.199 | 69.028 | 43.794 | 1.00 | 89.48 |
| 4775 | O | THR D | 25 | 48.404 | 67.956 | 44.359 | 1.00 | 89.48 |
| 4776 | N | CYS D | 26 | 48.909 | 70.117 | 44.050 | 1.00 | 125.74 |
| 4777 | CA | CYS D | 26 | 49.942 | 70.082 | 45.070 | 1.00 | 125.74 |
| 4778 | C | CYS D | 26 | 49.298 | 70.358 | 46.407 | 1.00 | 125.74 |
| 4779 | O | CYS D | 26 | 48.415 | 71.196 | 46.512 | 1.00 | 125.74 |
| 4780 | CB | CYS D | 26 | 51.034 | 71.118 | 44.810 | 1.00 | 105.78 |
| 4781 | SG | CYS D | 26 | 52.476 | 70.930 | 45.922 | 1.00 | 105.78 |
| 4782 | N | ASN D | 27 | 49.751 | 69.628 | 47.416 | 1.00 | 184.56 |
| 4783 | CA | ASN D | 27 | 49.263 | 69.743 | 48.776 | 1.00 | 184.56 |
| 4784 | CB | ASN D | 27 | 50.450 | 69.894 | 49.698 | 1.00 | 249.08 |
| 4785 | CG | ASN D | 27 | 50.107 | 69.554 | 51.100 | 1.00 | 249.08 |
| 4786 | OD1 | ASN D | 27 | 49.328 | 68.630 | 51.334 | 1.00 | 249.08 |
| 4787 | ND2 | ASN D | 27 | 50.683 | 70.281 | 52.054 | 1.00 | 249.08 |
| 4788 | C | ASN D | 27 | 48.283 | 70.880 | 49.023 | 1.00 | 184.56 |
| 4789 | O | ASN D | 27 | 48.686 | 71.988 | 49.365 | 1.00 | 184.56 |
| 4790 | N | GLY D | 28 | 46.995 | 70.600 | 48.844 | 1.00 | 249.39 |
| 4791 | CA | GLY D | 28 | 45.972 | 71.612 | 49.043 | 1.00 | 249.39 |
| 4792 | C | GLY D | 28 | 44.644 | 71.030 | 48.616 | 1.00 | 249.39 |
| 4793 | O | GLY D | 28 | 44.494 | 70.609 | 47.470 | 1.00 | 249.39 |
| 4794 | N | ASN D | 29 | 43.674 | 71.006 | 49.524 | 1.00 | 249.47 |
| 4795 | CA | ASN D | 29 | 42.377 | 70.429 | 49.206 | 1.00 | 249.47 |
| 4796 | CB | ASN D | 29 | 41.619 | 70.112 | 50.497 | 1.00 | 246.79 |
| 4797 | CG | ASN D | 29 | 40.397 | 69.249 | 50.256 | 1.00 | 246.79 |
| 4798 | OD1 | ASN D | 29 | 40.234 | 68.672 | 49.182 | 1.00 | 246.79 |
| 4799 | ND2 | ASN D | 29 | 39.536 | 69.146 | 51.262 | 1.00 | 246.79 |
| 4800 | C | ASN D | 29 | 41.494 | 71.261 | 48.274 | 1.00 | 249.47 |
| 4801 | O | ASN D | 29 | 41.005 | 70.746 | 47.265 | 1.00 | 249.47 |
| 4802 | N | ASN D | 30 | 41.294 | 72.538 | 48.594 | 1.00 | 206.51 |
| 4803 | CA | ASN D | 30 | 40.437 | 73.386 | 47.766 | 1.00 | 206.51 |
| 4804 | CB | ASN D | 30 | 39.137 | 73.705 | 48.518 | 1.00 | 210.57 |
| 4805 | CG | ASN D | 30 | 38.302 | 72.469 | 48.794 | 1.00 | 210.57 |
| 4806 | OD1 | ASN D | 30 | 37.878 | 72.232 | 49.926 | 1.00 | 210.57 |
| 4807 | ND2 | ASN D | 30 | 38.054 | 71.678 | 47.757 | 1.00 | 210.57 |
| 4808 | C | ASN D | 30 | 41.073 | 74.685 | 47.297 | 1.00 | 206.51 |
| 4809 | O | ASN D | 30 | 41.381 | 74.848 | 46.115 | 1.00 | 206.51 |
| 4810 | N | PHE D | 31 | 41.266 | 75.614 | 48.224 | 1.00 | 230.41 |
| 4811 | CA | PHE D | 31 | 41.829 | 76.899 | 47.860 | 1.00 | 230.41 |
| 4812 | CB | PHE D | 31 | 40.891 | 78.020 | 48.330 | 1.00 | 249.56 |
| 4813 | CG | PHE D | 31 | 39.472 | 77.878 | 47.828 | 1.00 | 249.56 |
| 4814 | CD1 | PHE D | 31 | 38.588 | 76.991 | 48.440 | 1.00 | 249.56 |
| 4815 | CD2 | PHE D | 31 | 39.030 | 78.608 | 46.725 | 1.00 | 249.56 |
| 4816 | CE1 | PHE D | 31 | 37.283 | 76.839 | 47.969 | 1.00 | 249.56 |
| 4817 | CE2 | PHE D | 31 | 37.727 | 78.463 | 46.245 | 1.00 | 249.56 |
| 4818 | CZ | PHE D | 31 | 36.853 | 77.574 | 46.866 | 1.00 | 249.56 |
| 4819 | C | PHE D | 31 | 43.249 | 77.132 | 48.360 | 1.00 | 230.41 |
| 4820 | O | PHE D | 31 | 43.542 | 76.994 | 49.552 | 1.00 | 230.41 |
| 4821 | N | PHE D | 32 | 44.122 | 77.486 | 47.416 | 1.00 | 186.13 |
| 4822 | CA | PHE D | 32 | 45.531 | 77.753 | 47.683 | 1.00 | 186.13 |
| 4823 | CB | PHE D | 32 | 46.392 | 76.753 | 46.925 | 1.00 | 237.35 |
| 4824 | CG | PHE D | 32 | 47.810 | 76.726 | 47.381 | 1.00 | 237.35 |
| 4825 | CD1 | PHE D | 32 | 48.121 | 76.283 | 48.659 | 1.00 | 237.35 |
| 4826 | CD2 | PHE D | 32 | 48.835 | 77.167 | 46.552 | 1.00 | 237.35 |
| 4827 | CE1 | PHE D | 32 | 49.434 | 76.277 | 49.112 | 1.00 | 237.35 |
| 4828 | CE2 | PHE D | 32 | 50.156 | 77.1 66 | 46.998 | 1.00 | 237.35 |
| 4829 | CZ | PHE D | 32 | 50.454 | 76.719 | 48.286 | 1.00 | 237.35 |
| 4830 | C | PHE D | 32 | 45.909 | 79.173 | 47.251 | 1.00 | 186.13 |
| 4831 | O | PHE D | 32 | 45.122 | 79.856 | 46.601 | 1.00 | 186.13 |
| 4832 | N | GLU D | 33 | 47.117 | 79.617 | 47.595 | 1.00 | 249.49 |
| 4833 | CA | GLU D | 33 | 47.539 | 80.969 | 47.225 | 1.00 | 249.49 |
| 4834 | CB | GLU D | 33 | 47.683 | 81.860 | 48.457 | 1.00 | 249.38 |
| 4835 | CG | GLU D | 33 | 47.919 | 83.321 | 48.090 | 1.00 | 249.38 |
| 4836 | CD | GLU D | 33 | 46.730 | 83.912 | 47.362 | 1.00 | 249.38 |
| 4837 | OE1 | GLU D | 33 | 45.593 | 83.561 | 47.740 | 1.00 | 249.38 |
| 4838 | OE2 | GLU D | 33 | 46.914 | 84.731 | 46.433 | 1.00 | 249.38 |
| 4839 | C | GLU D | 33 | 48.822 | 81.120 | 46.422 | 1.00 | 249.49 |
| 4840 | O | GLU D | 33 | 48.826 | 81.765 | 45.372 | 1.00 | 249.49 |
| 4841 | N | VAL D | 34 | 49.918 | 80.566 | 46.929 | 1.00 | 207.78 |
| 4842 | CA | VAL D | 34 | 51.194 | 80.698 | 46.247 | 1.00 | 207.78 |
| 4843 | CB | VAL D | 34 | 52.284 | 79.859 | 46.944 | 1.00 | 207.37 |
| 4844 | CG1 | VAL D | 34 | 53.608 | 80.005 | 46.212 | 1.00 | 207.37 |
| 4845 | CG2 | VAL D | 34 | 52.437 | 80.316 | 48.384 | 1.00 | 207.37 |
| 4846 | C | VAL D | 34 | 51.130 | 80.333 | 44.770 | 1.00 | 207.78 |
| 4847 | O | VAL D | 34 | 50.333 | 79.492 | 44.343 | 1.00 | 207.78 |
| 4848 | N | SER D | 35 | 51.966 | 81.007 | 43.992 | 1.00 | 228.15 |
| 4849 | CA | SER D | 35 | 52.043 | 80.778 | 42.563 | 1.00 | 228.15 |
| 4850 | CB | SER D | 35 | 51.944 | 82.104 | 41.810 | 1.00 | 249.21 |
| 4851 | OG | SER D | 35 | 53.093 | 82.901 | 42.038 | 1.00 | 249.21 |
| 4852 | C | SER D | 35 | 53.386 | 80.116 | 42.275 | 1.00 | 228.15 |
| 4853 | O | SER D | 35 | 53.703 | 79.813 | 41.126 | 1.00 | 228.15 |
| 4854 | N | SER D | 36 | 54.177 | 79.906 | 43.326 | 1.00 | 238.59 |
| 4855 | CA | SER D | 36 | 55.481 | 79.265 | 43.185 | 1.00 | 238.59 |
| 4856 | CB | SER D | 36 | 56.552 | 80.002 | 43.997 | 1.00 | 200.88 |
| 4857 | OG | SER D | 36 | 56.368 | 79.808 | 45.389 | 1.00 | 200.88 |
| 4858 | C | SER D | 36 | 55.395 | 77.821 | 43.653 | 1.00 | 238.59 |
| 4859 | O | SER D | 36 | 55.568 | 77.519 | 44.835 | 1.00 | 238.59 |
| 4860 | N | THR D | 37 | 55.115 | 76.935 | 42.706 | 1.00 | 119.38 |
| 4861 | CA | THR D | 37 | 55.004 | 75.514 | 42.974 | 1.00 | 119.38 |
| 4862 | CB | THR D | 37 | 53.561 | 75.034 | 42.738 | 1.00 | 138.47 |
| 4863 | OG1 | THR D | 37 | 52.664 | 75.794 | 43.557 | 1.00 | 138.47 |
| 4864 | CG2 | THR D | 37 | 53.426 | 73.568 | 43.078 | 1.00 | 138.47 |
| 4865 | C | THR D | 37 | 55.950 | 74.838 | 41.992 | 1.00 | 119.38 |
| 4866 | O | THR D | 37 | 56.054 | 75.258 | 40.841 | 1.00 | 119.38 |
| 4867 | N | LYS D | 38 | 56.653 | 73.808 | 42.446 | 1.00 | 140.44 |
| 4868 | CA | LYS D | 38 | 57.594 | 73.098 | 41.585 | 1.00 | 140.44 |
| 4869 | CB | LYS D | 38 | 58.938 | 72.982 | 42.288 | 1.00 | 200.36 |
| 4870 | CG | LYS D | 38 | 59.508 | 74.309 | 42.714 | 1.00 | 200.36 |
| 4371 | CD | LYS D | 38 | 60.837 | 74.125 | 43.415 | 1.00 | 200.36 |
| 4872 | CE | LYS D | 38 | 61.449 | 75.465 | 43.766 | 1.00 | 200.36 |
| 4873 | NZ | LYS D | 38 | 62.770 | 75.308 | 44.427 | 1.00 | 200.36 |
| 4874 | C | LYS D | 38 | 57.100 | 71.701 | 41.218 | 1.00 | 140.44 |
| 4875 | O | LYS D | 38 | 56.507 | 71.013 | 42.045 | 1.00 | 140.44 |
| 4876 | N | TRP D | 39 | 57.341 | 71.284 | 39.976 | 1.00 | 125.62 |
| 4877 | CA | TRP D | 39 | 56.934 | 69.953 | 39.520 | 1.00 | 125.62 |
| 4878 | CB | TRP D | 39 | 55.830 | 70.028 | 38.470 | 1.00 | 111.13 |
| 4879 | CG | TRP D | 39 | 54.540 | 70.582 | 38.973 | 1.00 | 111.13 |
| 4880 | CD2 | TRP D | 39 | 53.645 | 69.978 | 39.915 | 1.00 | 111.13 |
| 4881 | CE2 | TRP D | 39 | 52.567 | 70.867 | 40.090 | 1.00 | 111.13 |
| 4882 | CE3 | TRP D | 39 | 53.651 | 68.776 | 40.628 | 1.00 | 111.13 |
| 4883 | CD1 | TRP D | 39 | 53.984 | 71.774 | 38.628 | 1.00 | 111.13 |
| 4884 | NE1 | TRP D | 39 | 52.798 | 71.953 | 39.295 | 1.00 | 111.13 |
| 4885 | CZ2 | TRP D | 39 | 51.503 | 70.588 | 40.948 | 1.00 | 111.13 |
| 4886 | CZ3 | TRP D | 39 | 52.589 | 68.503 | 41.482 | 1.00 | 111.13 |
| 4887 | CH2 | TRP D | 39 | 51.531 | 69.405 | 41.633 | 1.00 | 111.13 |
| 4888 | C | TRP D | 39 | 58.115 | 69.240 | 38.913 | 1.00 | 125.62 |
| 4889 | O | TRP D | 39 | 58.809 | 69.797 | 38.077 | 1.00 | 125.62 |
| 4890 | N | PHE D | 40 | 58.331 | 68.000 | 39.318 | 1.00 | 94.48 |
| 4891 | CA | PHE D | 40 | 59.458 | 67.260 | 38.800 | 1.00 | 94.48 |
| 4892 | CB | PHE D | 40 | 60.475 | 66.976 | 39.910 | 1.00 | 162.61 |
| 4893 | CG | PHE D | 40 | 60.977 | 68.203 | 40.607 | 1.00 | 162.61 |
| 4894 | CD1 | PHE D | 40 | 60.217 | 68.816 | 41.594 | 1.00 | 162.61 |
| 4895 | CD2 | PHE D | 40 | 62.222 | 68.734 | 40.292 | 1.00 | 162.61 |
| 4896 | CE1 | PHE D | 40 | 60.687 | 69.941 | 42.264 | 1.00 | 162.61 |
| 4897 | CE2 | PHE D | 40 | 62.705 | 69.859 | 40.953 | 1.00 | 162.61 |
| 4898 | CZ | PHE D | 40 | 61.934 | 70.465 | 41.945 | 1.00 | 162.61 |
| 4899 | C | PHE D | 40 | 59.063 | 65.951 | 38.152 | 1.00 | 94.48 |
| 4900 | O | PHE D | 40 | 59.061 | 64.906 | 38.803 | 1.00 | 94.48 |
| 4901 | N | HIS D | 41 | 58.727 | 66.006 | 36.869 | 1.00 | 72.94 |
| 4902 | CA | HIS D | 41 | 58.368 | 64.797 | 36.133 | 1.00 | 72.94 |
| 4903 | CB | HIS D | 41 | 57.649 | 65.192 | 34.848 | 1.00 | 108.26 |
| 4904 | CG | HIS D | 41 | 57.262 | 64.034 | 33.991 | 1.00 | 108.26 |
| 4905 | CD2 | HIS D | 41 | 57.305 | 63.866 | 32.649 | 1.00 | 108.26 |
| 4906 | ND1 | HIS D | 41 | 56.717 | 62.879 | 34.507 | 1.00 | 108.26 |
| 4907 | CE1 | HIS D | 41 | 56.441 | 62.049 | 33.519 | 1.00 | 108.26 |
| 4908 | NE2 | HIS D | 41 | 56.788 | 62.625 | 32.381 | 1.00 | 108.26 |
| 4909 | C | HIS D | 41 | 59.642 | 63.973 | 35.816 | 1.00 | 72.94 |
| 4910 | O | HIS D | 41 | 60.482 | 64.384 | 35.014 | 1.00 | 72.94 |
| 4911 | N | ASN D | 42 | 59.770 | 62.805 | 36.445 | 1.00 | 79.39 |
| 4912 | CA | ASN D | 42 | 60.939 | 61.934 | 36.277 | 1.00 | 79.39 |
| 4913 | CB | ASN D | 42 | 61.153 | 61.529 | 34.808 | 1.00 | 100.05 |
| 4914 | CG | ASN D | 42 | 60.141 | 60.504 | 34.327 | 1.00 | 100.05 |
| 4915 | OD1 | ASN D | 42 | 58.962 | 60.635 | 34.637 | 1.00 | 100.05 |
| 4916 | ND2 | ASN D | 42 | 60.578 | 59.502 | 33.560 | 1.00 | 100.05 |
| 4917 | C | ASN D | 42 | 62.190 | 62.653 | 36.783 | 1.00 | 79.39 |
| 4918 | O | ASN D | 42 | 63.298 | 62.386 | 36.318 | 1.00 | 79.39 |
| 4919 | N | GLY D | 43 | 62.013 | 63.562 | 37.739 | 1.00 | 194.33 |
| 4920 | CA | GLY D | 43 | 63.147 | 64.294 | 38.279 | 1.00 | 194.33 |
| 4921 | C | GLY D | 43 | 63.397 | 65.624 | 37.584 | 1.00 | 194.33 |
| 4922 | O | GLY D | 43 | 63.744 | 66.616 | 38.226 | 1.00 | 194.33 |
| 4923 | N | SER D | 44 | 63.221 | 65.644 | 36.267 | 1.00 | 226.19 |
| 4924 | CA | SER D | 44 | 63.422 | 66.852 | 35.476 | 1.00 | 226.19 |
| 4925 | CB | SER D | 44 | 63.315 | 66.520 | 33.987 | 1.00 | 149.84 |
| 4926 | OG | SER D | 44 | 64.180 | 65.450 | 33.639 | 1.00 | 149.84 |
| 4927 | C | SER D | 44 | 62.376 | 67.899 | 35.837 | 1.00 | 226.19 |
| 4928 | O | SER D | 44 | 61.179 | 67.617 | 35.812 | 1.00 | 226.19 |
| 4929 | N | LEU D | 45 | 62.824 | 69.107 | 36.172 | 1.00 | 151.08 |
| 4930 | CA | LEU D | 45 | 61.897 | 70.180 | 36.531 | 1.00 | 151.08 |
| 4931 | CB | LEU D | 45 | 62.673 | 71.473 | 36.830 | 1.00 | 168.08 |
| 4932 | CG | LEU D | 45 | 61.854 | 72.699 | 37.259 | 1.00 | 168.08 |
| 4933 | CD1 | LEU D | 45 | 60.980 | 72.345 | 38.451 | 1.00 | 168.08 |
| 4934 | CD2 | LEU D | 45 | 62.788 | 73.853 | 37.607 | 1.00 | 168.08 |
| 4935 | C | LEU D | 45 | 60.885 | 70.422 | 35.398 | 1.00 | 151.03 |
| 4936 | O | LEU D | 45 | 61.215 | 70.281 | 34.219 | 1.00 | 151.08 |
| 4937 | N | SER D | 46 | 59.650 | 70.772 | 35.755 | 1.00 | 118.65 |
| 4938 | CA | SER D | 46 | 58.614 | 71.032 | 34.762 | 1.00 | 118.65 |
| 4939 | CB | SER D | 46 | 57.279 | 70.467 | 35.236 | 1.00 | 145.92 |
| 4940 | OG | SER D | 46 | 56.288 | 70.641 | 34.240 | 1.00 | 145.92 |
| 4941 | C | SER D | 46 | 58.499 | 72.537 | 34.569 | 1.00 | 118.65 |
| 4942 | O | SER D | 46 | 59.012 | 73.306 | 35.378 | 1.00 | 118.65 |
| 4943 | N | GLU D | 47 | 57.828 | 72.965 | 33.505 | 1.00 | 181.92 |
| 4944 | CA | GLU D | 47 | 57.679 | 74.395 | 33.255 | 1.00 | 181.92 |
| 4945 | CB | GLU D | 47 | 57.725 | 74.692 | 31.746 | 1.00 | 232.55 |
| 4946 | CG | GLU D | 47 | 58.747 | 73.882 | 30.951 | 1.00 | 232.55 |
| 4947 | CD | GLU D | 47 | 58.494 | 73.918 | 29.437 | 1.00 | 232.55 |
| 4948 | OE1 | GLU D | 47 | 57.657 | 73.130 | 28.947 | 1.00 | 232.55 |
| 4949 | OE2 | GLU D | 47 | 59.123 | 74.749 | 28.747 | 1.00 | 232.55 |
| 4950 | C | GLU D | 47 | 56.398 | 74.999 | 33.858 | 1.00 | 181.92 |
| 4951 | O | GLU D | 47 | 56.185 | 76.203 | 33.732 | 1.00 | 181.92 |
| 4952 | N | GLU D | 48 | 55.538 | 74.196 | 34.492 | 1.00 | 100.90 |
| 4953 | CA | GLU D | 48 | 54.330 | 74.777 | 35.104 | 1.00 | 100.90 |
| 4954 | CB | GLU D | 48 | 53.184 | 73.753 | 35.227 | 1.00 | 175.62 |
| 4955 | CG | GLU D | 48 | 51.945 | 74.246 | 36.025 | 1.00 | 175.62 |
| 4956 | CD | GLU D | 48 | 51.173 | 75.384 | 35.355 | 1.00 | 175.62 |
| 4957 | OE1 | GLU D | 48 | 50.544 | 75.147 | 34.300 | 1.00 | 175.62 |
| 4958 | OE2 | GLU D | 48 | 51.193 | 76.517 | 35.887 | 1.00 | 175.62 |
| 4959 | C | GLU D | 48 | 54.698 | 75.309 | 36.490 | 1.00 | 100.90 |
| 4960 | O | GLU D | 48 | 55.679 | 74.868 | 37.096 | 1.00 | 100.90 |
| 4961 | N | THR D | 49 | 53.927 | 76.275 | 36.977 | 1.00 | 145.22 |
| 4962 | CA | THR D | 49 | 54.186 | 76.860 | 38.287 | 1.00 | 145.22 |
| 4963 | CB | THR D | 49 | 54.710 | 78.308 | 38.155 | 1.00 | 156.90 |
| 4964 | OG1 | THR D | 49 | 53.774 | 79.094 | 37.405 | 1.00 | 156.90 |
| 4965 | CG2 | THR D | 49 | 56.059 | 78.319 | 37.442 | 1.00 | 156.90 |
| 4966 | C | THR D | 49 | 52.921 | 76.841 | 39.150 | 1.00 | 145.22 |
| 4967 | O | THR D | 49 | 53.002 | 76.780 | 40.384 | 1.00 | 145.22 |
| 4968 | N | ASN D | 50 | 51.757 | 76.881 | 38.500 | 1.00 | 138.33 |
| 4969 | CA | ASN D | 50 | 50.486 | 76.844 | 39.213 | 1.00 | 138.33 |
| 4970 | CB | ASN D | 50 | 49.323 | 76.826 | 38.220 | 1.00 | 234.43 |
| 4971 | CG | ASN D | 50 | 47.991 | 77.129 | 38.880 | 1.00 | 234.43 |
| 4972 | OD1 | ASN D | 50 | 47.868 | 77.026 | 40.100 | 1.00 | 234.43 |
| 4973 | ND2 | ASN D | 50 | 46.989 | 77.490 | 38.081 | 1.00 | 234.43 |
| 4974 | C | ASN D | 50 | 50.480 | 75.564 | 40.054 | 1.00 | 138.33 |
| 4975 | O | ASN D | 50 | 51.104 | 74.574 | 39.683 | 1.00 | 138.33 |
| 4976 | N | SER D | 51 | 49.782 | 75.578 | 41.183 | 1.00 | 192.24 |
| 4977 | CA | SER D | 51 | 49.733 | 74.400 | 42.046 | 1.00 | 192.24 |
| 4978 | CB | SER D | 51 | 49.042 | 74.734 | 43.376 | 1.00 | 111.18 |
| 4979 | OG | SER D | 51 | 47.655 | 74.994 | 43.211 | 1.00 | 111.18 |
| 4980 | C | SER D | 51 | 49.023 | 73.213 | 41.399 | 1.00 | 192.24 |
| 4981 | O | SER D | 51 | 49.166 | 72.078 | 41.848 | 1.00 | 192.24 |
| 4982 | N | SER D | 52 | 48.252 | 73.470 | 40.351 | 1.00 | 99.05 |
| 4983 | CA | SER D | 52 | 47.526 | 72.400 | 39.677 | 1.00 | 99.05 |
| 4984 | CB | SER D | 52 | 46.041 | 72.765 | 39.522 | 1.00 | 120.15 |
| 4985 | OG | SER D | 52 | 45.402 | 72.880 | 40.781 | 1.00 | 120.15 |
| 4986 | C | SER D | 52 | 48.147 | 72.145 | 38.314 | 1.00 | 99.05 |
| 4987 | O | SER D | 52 | 48.052 | 72.970 | 37.410 | 1.00 | 99.05 |
| 4988 | N | LEU D | 53 | 48.797 | 70.997 | 38.183 | 1.00 | 107.39 |
| 4989 | CA | LEU D | 53 | 49.443 | 70.602 | 36.938 | 1.00 | 107.39 |
| 4990 | CB | LEU D | 53 | 50.774 | 69.910 | 37.246 | 1.00 | 80.33 |
| 4991 | CG | LEU D | 53 | 51.398 | 68.972 | 36.203 | 1.00 | 80.33 |
| 4992 | CD1 | LEU D | 53 | 51.298 | 69.580 | 34.817 | 1.00 | 80.33 |
| 4993 | CD2 | LEU D | 53 | 52.856 | 68.675 | 36.589 | 1.00 | 80.33 |
| 4994 | C | LEU D | 53 | 48.543 | 69.654 | 36.158 | 1.00 | 107.39 |
| 4995 | O | LEU D | 53 | 48.472 | 68.474 | 36.476 | 1.00 | 107.39 |
| 4996 | N | ASN D | 54 | 47.876 | 70.159 | 35.130 | 1.00 | 103.21 |
| 4997 | CA | ASN D | 54 | 46.989 | 69.314 | 34.339 | 1.00 | 103.21 |
| 4998 | CB | ASN D | 54 | 45.977 | 70.162 | 33.573 | 1.00 | 126.61 |
| 4999 | CG | ASN D | 54 | 44.932 | 70.755 | 34.475 | 1.00 | 126.61 |
| 5000 | OD1 | ASN D | 54 | 44.260 | 70.038 | 35.217 | 1.00 | 126.61 |
| 5001 | ND2 | ASN D | 54 | 44.781 | 72.072 | 34.420 | 1.00 | 126.61 |
| 5002 | C | ASN D | 54 | 47.732 | 68.434 | 33.362 | 1.00 | 103.21 |
| 5003 | O | ASN D | 54 | 48.882 | 68.690 | 33.026 | 1.00 | 103.21 |
| 5004 | N | ILE D | 55 | 47.056 | 67.381 | 32.921 | 1.00 | 179.18 |
| 5005 | CA | ILE D | 55 | 47.601 | 66.449 | 31.947 | 1.00 | 179.18 |
| 5006 | CB | ILE D | 55 | 48.061 | 65.127 | 32.606 | 1.00 | 94.31 |
| 5007 | CG2 | ILE D | 55 | 48.187 | 64.027 | 31.553 | 1.00 | 94.31 |
| 5008 | CG1 | ILE D | 55 | 49.393 | 65.356 | 33.324 | 1.00 | 94.31 |
| 5009 | CD1 | ILE D | 55 | 49.946 | 64.146 | 34.029 | 1.00 | 94.31 |
| 5010 | C | ILE D | 55 | 46.473 | 66.173 | 30.975 | 1.00 | 179.18 |
| 5011 | O | ILE D | 55 | 45.402 | 65.719 | 31.373 | 1.00 | 179.18 |
| 5012 | N | VAL D | 56 | 46.701 | 66.476 | 29.704 | 1.00 | 148.36 |
| 5013 | CA | VAL D | 56 | 45.674 | 66.254 | 28.704 | 1.00 | 148.36 |
| 5014 | CB | VAL D | 56 | 45.589 | 67.433 | 27.737 | 1.00 | 191.19 |
| 5015 | CG1 | VAL D | 56 | 44.260 | 67.393 | 26.992 | 1.00 | 191.19 |
| 5016 | CG2 | VAL D | 56 | 45.729 | 68.734 | 28.509 | 1.00 | 191.19 |
| 5017 | C | VAL D | 56 | 45.998 | 64.975 | 27.956 | 1.00 | 148.36 |
| 5018 | O | VAL D | 56 | 46.797 | 64.180 | 28.445 | 1.00 | 148.36 |
| 5019 | N | ASN D | 57 | 45.386 | 64.774 | 26.789 | 1.00 | 142.96 |
| 5020 | CA | ASN D | 57 | 45.604 | 63.560 | 25.999 | 1.00 | 142.96 |
| 5021 | CB | ASN D | 57 | 45.673 | 63.895 | 24.509 | 1.00 | 249.24 |
| 5022 | CG | ASN D | 57 | 44.331 | 64.323 | 23.952 | 1.00 | 249.24 |
| 5023 | OD1 | ASN D | 57 | 43.331 | 63.617 | 24.101 | 1.00 | 249.24 |
| 5024 | ND2 | ASN D | 57 | 44.298 | 65.482 | 23.307 | 1.00 | 249.24 |
| 5025 | C | ASN D | 57 | 46.861 | 62.815 | 26.436 | 1.00 | 142.96 |
| 5026 | O | ASN D | 57 | 47.956 | 63.052 | 25.919 | 1.00 | 142.96 |
| 5027 | N | ALA D | 58 | 46.683 | 61.917 | 27.403 | 1.00 | 155.81 |
| 5028 | CA | ALA D | 58 | 47.775 | 61.140 | 27.966 | 1.00 | 155.81 |
| 5029 | CB | ALA D | 58 | 47.245 | 60.191 | 29.002 | 1.00 | 45.44 |
| 5030 | C | ALA D | 58 | 48.595 | 60.375 | 26.939 | 1.00 | 155.81 |
| 5031 | O | ALA D | 58 | 48.086 | 59.524 | 26.214 | 1.00 | 155.81 |
| 5032 | N | LYS D | 59 | 49.881 | 60.690 | 26.890 | 1.00 | 75.94 |
| 5033 | CA | LYS D | 59 | 50.807 | 60.036 | 25.979 | 1.00 | 75.94 |
| 5034 | CB | LYS D | 59 | 51.654 | 61.085 | 25.248 | 1.00 | 205.66 |
| 5035 | CG | LYS D | 59 | 50.830 | 62.088 | 24.439 | 1.00 | 205.66 |
| 5036 | CD | LYS D | 59 | 51.689 | 63.187 | 23.829 | 1.00 | 205.66 |
| 5037 | CE | LYS D | 59 | 50.838 | 64.174 | 23.036 | 1.00 | 205.66 |
| 5038 | NZ | LYS D | 59 | 51.652 | 65.278 | 22.452 | 1.00 | 205.66 |
| 5039 | C | LYS D | 59 | 51.673 | 59.164 | 26.886 | 1.00 | 75.94 |
| 5040 | O | LYS D | 59 | 51.945 | 59.548 | 28.028 | 1.00 | 75.94 |
| 5041 | N | PHE D | 60 | 52.082 | 57.991 | 26.398 | 1.00 | 73.11 |
| 5042 | CA | PHE D | 60 | 52.916 | 57.070 | 27.183 | 1.00 | 73.11 |
| 5043 | CB | PHE D | 60 | 53.521 | 56.025 | 26.277 | 1.00 | 111.86 |
| 5044 | CG | PHE D | 60 | 52.512 | 55.131 | 25.662 | 1.00 | 111.86 |
| 5045 | CD1 | PHE D | 60 | 52.745 | 54.540 | 24.431 | 1.00 | 111.86 |
| 5046 | CD2 | PHE D | 60 | 51.323 | 54.863 | 26.314 | 1.00 | 111.86 |
| 5047 | CE1 | PHE D | 60 | 51.810 | 53.690 | 23.853 | 1.00 | 111.86 |
| 5048 | CE2 | PHE D | 60 | 50.379 | 54.018 | 25.750 | 1.00 | 111.86 |
| 5049 | CZ | PHE D | 60 | 50.622 | 53.429 | 24.517 | 1.00 | 111.86 |
| 5050 | C | PHE D | 60 | 54.026 | 57.782 | 27.946 | 1.00 | 73.11 |
| 5051 | O | PHE D | 60 | 54.401 | 57.368 | 29.037 | 1.00 | 73.11 |
| 5052 | N | GLU D | 61 | 54.544 | 58.863 | 27.369 | 1.00 | 133.04 |
| 5053 | CA | GLU D | 61 | 55.611 | 59.639 | 27.993 | 1.00 | 133.04 |
| 5054 | CB | GLU D | 61 | 56.112 | 60.736 | 27.046 | 1.00 | 249.40 |
| 5055 | CG | GLU D | 61 | 56.707 | 60.232 | 25.750 | 1.00 | 249.40 |
| 5056 | CD | GLU D | 61 | 55.711 | 59.447 | 24.926 | 1.00 | 249.40 |
| 5057 | OE1 | GLU D | 61 | 54.616 | 59.981 | 24.643 | 1.00 | 249.40 |
| 5058 | OE2 | GLU D | 61 | 56.023 | 58.296 | 24.561 | 1.00 | 249.40 |
| 5059 | C | GLU D | 61 | 55.162 | 60.298 | 29.289 | 1.00 | 133.04 |
| 5060 | O | GLU D | 61 | 55.995 | 60.638 | 30.124 | 1.00 | 133.04 |
| 5061 | N | ASP D | 62 | 53.854 | 60.493 | 29.452 | 1.00 | 85.82 |
| 5062 | CA | ASP D | 62 | 53.344 | 61.130 | 30.659 | 1.00 | 85.82 |
| 5063 | CB | ASP D | 62 | 51.887 | 61.546 | 30.471 | 1.00 | 150.56 |
| 5064 | CG | ASP D | 62 | 51.694 | 62.436 | 29.258 | 1.00 | 150.56 |
| 5065 | OD1 | ASP D | 62 | 52.584 | 63.274 | 28.983 | 1.00 | 150.56 |
| 5066 | OD2 | ASP D | 62 | 50.650 | 62.302 | 28.585 | 1.00 | 150.56 |
| 5067 | C | ASP D | 62 | 53.484 | 60.179 | 31.838 | 1.00 | 85.82 |
| 5068 | O | ASP D | 62 | 53.464 | 60.605 | 32.999 | 1.00 | 85.82 |
| 5069 | N | SER D | 63 | 53.636 | 58.888 | 31.532 | 1.00 | 75.53 |
| 5070 | CA | SER D | 63 | 53.798 | 57.658 | 32.566 | 1.00 | 75.53 |
| 5071 | CB | SER D | 63 | 53.958 | 56.471 | 31.933 | 1.00 | 120.69 |
| 5072 | OG | SER D | 63 | 52.776 | 56.060 | 31.271 | 1.00 | 120.69 |
| 5073 | C | SER D | 63 | 55.064 | 58.219 | 33.303 | 1.00 | 75.53 |
| 5074 | O | SER D | 63 | 56.071 | 58.440 | 32.671 | 1.00 | 75.53 |
| 5075 | N | GLY D | 64 | 55.038 | 58.291 | 34.624 | 1.00 | 67.79 |
| 5076 | CA | GLY D | 64 | 56.262 | 58.644 | 35.318 | 1.00 | 67.79 |
| 5077 | C | GLY D | 64 | 56.113 | 58.959 | 36.798 | 1.00 | 67.79 |
| 5078 | O | GLY D | 64 | 55.060 | 58.708 | 37.391 | 1.00 | 67.79 |
| 5079 | N | GLU D | 65 | 57.173 | 59.500 | 37.396 | 1.00 | 63.91 |
| 5080 | CA | GLU D | 65 | 57.212 | 59.843 | 38.817 | 1.00 | 63.91 |
| 5081 | CB | GLU D | 65 | 58.542 | 59.353 | 39.392 | 1.00 | 198.27 |
| 5082 | CG | GLU D | 65 | 58.869 | 59.830 | 40.778 | 1.00 | 198.27 |
| 5083 | CD | GLU D | 65 | 60.319 | 59.576 | 41.129 | 1.00 | 198.27 |
| 5084 | OE1 | GLU D | 65 | 61.203 | 60.162 | 40.465 | 1.00 | 198.27 |
| 5085 | OE2 | GLU D | 65 | 60.576 | 58.786 | 42.061 | 1.00 | 198.27 |
| 5086 | C | GLU D | 65 | 57.098 | 61.355 | 38.943 | 1.00 | 63.91 |
| 5087 | O | GLU D | 65 | 57.862 | 62.078 | 38.322 | 1.00 | 63.91 |
| 5088 | N | TYR D | 66 | 56.151 | 61.849 | 39.727 | 1.00 | 104.89 |
| 5089 | CA | TYR D | 66 | 55.995 | 63.295 | 39.869 | 1.00 | 104.89 |
| 5090 | CB | TYR D | 66 | 54.621 | 63.739 | 39.384 | 1.00 | 61.05 |
| 5091 | CG | TYR D | 66 | 54.348 | 63.543 | 37.922 | 1.00 | 61.05 |
| 5092 | CD1 | TYR D | 66 | 54.057 | 62.291 | 37.403 | 1.00 | 61.05 |
| 5093 | CE1 | TYR D | 66 | 53.721 | 62.142 | 36.065 | 1.00 | 61.05 |
| 5094 | CD2 | TYR D | 66 | 54.307 | 64.633 | 37.065 | 1.00 | 61.05 |
| 5095 | CE2 | TYR D | 66 | 53.972 | 64.489 | 35.737 | 1.00 | 61.05 |
| 5096 | CZ | TYR D | 66 | 53.679 | 63.253 | 35.239 | 1.00 | 61.05 |
| 5097 | OH | TYR D | 66 | 53.338 | 63.149 | 33.911 | 1.00 | 61.05 |
| 5098 | C | TYR D | 66 | 56.119 | 63.743 | 41.314 | 1.00 | 104.89 |
| 5099 | O | TYR D | 66 | 55.990 | 62.923 | 42.228 | 1.00 | 104.89 |
| 5100 | N | LYS D | 67 | 56.343 | 65.046 | 41.518 | 1.00 | 107.31 |
| 5101 | CA | LYS D | 67 | 56.437 | 65.618 | 42.867 | 1.00 | 107.31 |
| 5102 | CB | LYS D | 67 | 57.700 | 65.134 | 43.562 | 1.00 | 121.51 |
| 5103 | CG | LYS D | 67 | 58.926 | 65.307 | 42.721 | 1.00 | 121.51 |
| 5104 | CD | LYS D | 67 | 60.124 | 64.712 | 43.416 | 1.00 | 121.51 |
| 5105 | CE | LYS D | 67 | 61.319 | 64.708 | 42.491 | 1.00 | 121.51 |
| 5106 | NZ | LYS D | 67 | 62.482 | 64.056 | 43.140 | 1.00 | 121.51 |
| 5107 | C | LYS D | 67 | 56.419 | 67.137 | 42.839 | 1.00 | 107.31 |
| 5108 | O | LYS D | 67 | 56.758 | 67.744 | 41.836 | 1.00 | 107.31 |
| 5109 | N | CYS D | 68 | 55.994 | 67.747 | 43.937 | 1.00 | 110.81 |
| 5110 | CA | CYS D | 68 | 55.962 | 69.190 | 44.011 | 1.00 | 110.81 |
| 5111 | C | CYS D | 68 | 56.694 | 69.634 | 45.260 | 1.00 | 110.81 |
| 5112 | O | CYS D | 68 | 56.922 | 68.842 | 46.170 | 1.00 | 110.81 |
| 5113 | CB | CYS D | 68 | 54.518 | 69.727 | 43.988 | 1.00 | 140.31 |
| 5114 | SG | CYS D | 68 | 53.384 | 69.216 | 45.310 | 1.00 | 140.31 |
| 5115 | N | GLN D | 69 | 57.093 | 70.900 | 45.274 | 1.00 | 126.93 |
| 5116 | CA | GLN D | 69 | 57.804 | 71.490 | 46.396 | 1.00 | 126.93 |
| 5117 | CB | GLN D | 69 | 59.300 | 71.191 | 46.281 | 1.00 | 112.91 |
| 5118 | CG | GLN D | 69 | 60.185 | 72.102 | 47.115 | 1.00 | 112.91 |
| 5119 | CD | GLN D | 69 | 61.665 | 71.867 | 46.875 | 1.00 | 112.91 |
| 5120 | OE1 | GLN D | 69 | 62.131 | 71.871 | 45.730 | 1.00 | 112.91 |
| 5121 | NE2 | GLN D | 69 | 62.416 | 71.667 | 47.959 | 1.00 | 112.91 |
| 5122 | C | GLN D | 69 | 57.566 | 72.990 | 46.341 | 1.00 | 126.93 |
| 5123 | O | GLN D | 69 | 57.314 | 73.542 | 45.269 | 1.00 | 126.93 |
| 5124 | N | HIS D | 70 | 57.642 | 73.652 | 47.488 | 1.00 | 191.71 |
| 5125 | CA | HIS D | 70 | 57.429 | 75.090 | 47.528 | 1.00 | 191.71 |
| 5126 | CB | HIS D | 70 | 56.372 | 75.434 | 48.577 | 1.00 | 178.35 |
| 5127 | CG | HIS D | 70 | 54.997 | 74.961 | 48.219 | 1.00 | 178.35 |
| 5128 | CD2 | HIS D | 70 | 54.315 | 73.848 | 48.585 | 1.00 | 178.35 |
| 5129 | ND1 | HIS D | 70 | 54.173 | 75.648 | 47.353 | 1.00 | 178.35 |
| 5130 | CE1 | HIS D | 70 | 53.041 | 74.981 | 47.202 | 1.00 | 178.35 |
| 5131 | NE2 | HIS D | 70 | 53.101 | 73.886 | 47.940 | 1.00 | 178.35 |
| 5132 | C | HIS D | 70 | 58.711 | 75.856 | 47.808 | 1.00 | 191.71 |
| 5133 | O | HIS D | 70 | 59.813 | 75.299 | 47.784 | 1.00 | 191.71 |
| 5134 | N | GLN D | 71 | 58.553 | 77.145 | 48.068 | 1.00 | 249.37 |
| 5135 | CA | GLN D | 71 | 59.681 | 78.018 | 48.338 | 1.00 | 249.37 |
| 5136 | CB | GLN D | 71 | 59.161 | 79.419 | 48.681 | 1.00 | 212.46 |
| 5137 | CG | GLN D | 71 | 60.101 | 80.540 | 48.260 | 1.00 | 212.46 |
| 5138 | CD | GLN D | 71 | 60.527 | 80.432 | 46.806 | 1.00 | 212.46 |
| 5139 | OE1 | GLN D | 71 | 59.736 | 80.661 | 45.894 | 1.00 | 212.46 |
| 5140 | NE2 | GLN D | 71 | 61.786 | 80.066 | 46.587 | 1.00 | 212.46 |
| 5141 | C | GLN D | 71 | 60.570 | 77.474 | 49.462 | 1.00 | 249.37 |
| 5142 | O | GLN D | 71 | 61.775 | 77.297 | 49.269 | 1.00 | 249.37 |
| 5143 | N | GLN D | 72 | 59.974 | 77.195 | 50.622 | 1.00 | 156.64 |
| 5144 | CA | GLN D | 72 | 60.728 | 76.683 | 51.771 | 1.00 | 156.64 |
| 5145 | CB | GLN D | 72 | 60.738 | 77.728 | 52.895 | 1.00 | 249.31 |
| 5146 | CG | GLN D | 72 | 61.596 | 77.360 | 54.104 | 1.00 | 249.31 |
| 5147 | CD | GLN D | 72 | 61.612 | 78.445 | 55.168 | 1.00 | 249.31 |
| 5148 | OE1 | GLN D | 72 | 62.001 | 79.584 | 54.904 | 1.00 | 249.31 |
| 5149 | NE2 | GLN D | 72 | 61.187 | 78.096 | 56.378 | 1.00 | 249.31 |
| 5150 | C | GLN D | 72 | 60.149 | 75.374 | 52.302 | 1.00 | 156.64 |
| 5151 | O | GLN D | 72 | 59.772 | 75.277 | 53.472 | 1.00 | 156.64 |
| 5152 | N | VAL D | 73 | 60.084 | 74.362 | 51.446 | 1.00 | 234.28 |
| 5153 | CA | VAL D | 73 | 59.530 | 73.078 | 51.852 | 1.00 | 234.28 |
| 5154 | CB | VAL D | 73 | 58.026 | 73.002 | 51.529 | 1.00 | 131.77 |
| 5155 | CG1 | VAL D | 73 | 57.398 | 71.832 | 52.244 | 1.00 | 131.77 |
| 5156 | CG2 | VAL D | 73 | 57.350 | 74.292 | 51.910 | 1.00 | 131.77 |
| 5157 | C | VAL D | 73 | 60.224 | 71.930 | 51.141 | 1.00 | 234.28 |
| 5158 | O | VAL D | 73 | 60.652 | 72.060 | 49.995 | 1.00 | 234.28 |
| 5159 | N | ASN D | 74 | 60.337 | 70.802 | 51.824 | 1.00 | 160.29 |
| 5160 | CA | ASN D | 74 | 60.971 | 69.646 | 51.228 | 1.00 | 160.29 |
| 5161 | CB | ASN D | 74 | 61.437 | 68.687 | 52.321 | 1.00 | 140.71 |
| 5162 | CG | ASN D | 74 | 62.337 | 69.366 | 53.332 | 1.00 | 140.71 |
| 5163 | OD1 | ASN D | 74 | 63.216 | 70.152 | 52.957 | 1.00 | 140.71 |
| 5164 | ND2 | ASN D | 74 | 62.130 | 69.065 | 54.613 | 1.00 | 140.71 |
| 5165 | C | ASN D | 74 | 59.985 | 68.964 | 50.286 | 1.00 | 160.29 |
| 5166 | O | ASN D | 74 | 58.839 | 68.692 | 50.653 | 1.00 | 160.29 |
| 5167 | N | GLU D | 75 | 60.446 | 68.711 | 49.064 | 1.00 | 155.73 |
| 5168 | CA | GLU D | 75 | 59.644 | 68.074 | 48.028 | 1.00 | 155.73 |
| 5169 | CB | GLU D | 75 | 60.555 | 67.631 | 46.881 | 1.00 | 134.99 |
| 5170 | CG | GLU D | 75 | 61.940 | 67.190 | 47.322 | 1.00 | 134.99 |
| 5171 | CD | GLU D | 75 | 62.884 | 66.978 | 46.149 | 1.00 | 134.99 |
| 5172 | OE1 | GLU D | 75 | 63.056 | 67.916 | 45.340 | 1.00 | 134.99 |
| 5173 | OE2 | GLU D | 75 | 63.460 | 65.875 | 46.038 | 1.00 | 134.99 |
| 5174 | C | GLU D | 75 | 58.794 | 66.907 | 48.520 | 1.00 | 155.73 |
| 5175 | O | GLU D | 75 | 59.207 | 66.137 | 49.385 | 1.00 | 155.73 |
| 5176 | N | SER D | 76 | 57.601 | 66.792 | 47.946 | 1.00 | 91.19 |
| 5177 | CA | SER D | 76 | 56.635 | 65.760 | 48.302 | 1.00 | 91.19 |
| 5178 | CB | SER D | 76 | 55.314 | 66.052 | 47.619 | 1.00 | 86.66 |
| 5179 | OG | SER D | 76 | 55.477 | 65.917 | 46.224 | 1.00 | 86.66 |
| 5180 | C | SER D | 76 | 57.050 | 64.349 | 47.909 | 1.00 | 91.19 |
| 5181 | O | SER D | 76 | 57.892 | 64.165 | 47.020 | 1.00 | 91.19 |
| 5182 | N | GLU D | 77 | 56.431 | 63.362 | 48.562 | 1.00 | 100.72 |
| 5183 | CA | GLU D | 77 | 56.701 | 61.959 | 48.272 | 1.00 | 100.72 |
| 5184 | CB | GLU D | 77 | 55.971 | 61.046 | 49.259 | 1.00 | 188.13 |
| 5185 | CG | GLU D | 77 | 56.457 | 61.191 | 50.694 | 1.00 | 188.13 |
| 5186 | CD | GLU D | 77 | 57.912 | 60.781 | 50.876 | 1.00 | 188.13 |
| 5187 | OE1 | GLU D | 77 | 58.637 | 60.666 | 49.867 | 1.00 | 188.13 |
| 5188 | OE2 | GLU D | 77 | 58.343 | 60.589 | 52.036 | 1.00 | 188.13 |
| 5189 | C | GLU D | 77 | 56.203 | 61.715 | 46.857 | 1.00 | 100.72 |
| 5190 | O | GLU D | 77 | 55.012 | 61.851 | 46.588 | 1.00 | 100.72 |
| 5191 | N | PRO D | 78 | 57.108 | 61.380 | 45.928 | 1.00 | 89.58 |
| 5192 | CD | PRO D | 78 | 58.577 | 61.416 | 46.096 | 1.00 | 142.24 |
| 5193 | CA | PRO D | 78 | 56.752 | 61.125 | 44.532 | 1.00 | 89.58 |
| 5194 | CB | PRO D | 78 | 58.018 | 60.508 | 43.955 | 1.00 | 142.24 |
| 5195 | CG | PRO D | 78 | 59.097 | 61.270 | 44.669 | 1.00 | 142.24 |
| 5196 | C | PRO D | 78 | 55.529 | 60.249 | 44.351 | 1.00 | 89.58 |
| 5197 | O | PRO D | 78 | 55.169 | 59.471 | 45.234 | 1.00 | 89.58 |
| 5198 | N | VAL D | 79 | 54.889 | 60.397 | 43.201 | 1.00 | 92.62 |
| 5199 | CA | VAL D | 79 | 53.713 | 59.620 | 42.893 | 1.00 | 92.62 |
| 5200 | CB | VAL D | 79 | 52.466 | 60.484 | 42.999 | 1.00 | 66.56 |
| 5201 | CG1 | VAL D | 79 | 51.284 | 59.785 | 42.333 | 1.00 | 66.56 |
| 5202 | CG2 | VAL D | 79 | 52.177 | 60.763 | 44.458 | 1.00 | 66.56 |
| 5203 | C | VAL D | 79 | 53.834 | 59.092 | 41.483 | 1.00 | 92.62 |
| 5204 | O | VAL D | 79 | 54.122 | 59.880 | 40.566 | 1.00 | 92.62 |
| 5205 | N | TYR D | 80 | 53.625 | 57.782 | 41.295 | 1.00 | 61.19 |
| 5206 | CA | TYR D | 80 | 53.757 | 57.256 | 39.952 | 1.00 | 61.19 |
| 5207 | CB | TYR D | 80 | 54.372 | 55.878 | 39.936 | 1.00 | 249.26 |
| 5208 | CG | TYR D | 80 | 54.869 | 55.534 | 38.557 | 1.00 | 249.26 |
| 5209 | CD1 | TYR D | 80 | 55.895 | 56.275 | 37.979 | 1.00 | 249.26 |
| 5210 | CE1 | TYR D | 80 | 56.370 | 55.971 | 36.708 | 1.00 | 249.26 |
| 5211 | CD2 | TYR D | 80 | 54.335 | 54.491 | 37.816 | 1.00 | 249.26 |
| 5212 | CE2 | TYR D | 80 | 54.829 | 54.208 | 36.532 | 1.00 | 249.26 |
| 5213 | CZ | TYR D | 80 | 55.822 | 54.920 | 35.993 | 1.00 | 249.26 |
| 5214 | OH | TYR D | 80 | 56.359 | 54.647 | 34.755 | 1.00 | 249.26 |
| 5215 | C | TYR D | 80 | 52.471 | 57.184 | 39.194 | 1.00 | 61.19 |
| 5216 | O | TYR D | 80 | 51.448 | 56.804 | 39.737 | 1.00 | 61.19 |
| 5217 | N | LEU D | 81 | 52.529 | 57.540 | 37.924 | 1.00 | 59.82 |
| 5218 | CA | LEU D | 81 | 51.354 | 57.492 | 37.090 | 1.00 | 59.82 |
| 5219 | CB | LEU D | 81 | 51.089 | 58.875 | 36.535 | 1.00 | 66.30 |
| 5220 | CG | LEU D | 81 | 49.972 | 58.868 | 35.515 | 1.00 | 66.30 |
| 5221 | CD1 | LEU D | 81 | 48.705 | 58.435 | 36.202 | 1.00 | 66.30 |
| 5222 | CD2 | LEU D | 81 | 49.808 | 60.240 | 34.932 | 1.00 | 66.30 |
| 5223 | C | LEU D | 81 | 51.664 | 56.531 | 35.945 | 1.00 | 59.82 |
| 5224 | O | LEU D | 81 | 52.715 | 56.663 | 35.333 | 1.00 | 59.82 |
| 5225 | N | GLU D | 82 | 50.795 | 55.561 | 35.658 | 1.00 | 81.20 |
| 5226 | CA | GLU D | 82 | 51.069 | 54.640 | 34.557 | 1.00 | 81.20 |
| 5227 | CB | GLU D | 82 | 51.229 | 53.211 | 35.072 | 1.00 | 125.93 |
| 5228 | CG | GLU D | 82 | 52.081 | 52.353 | 34.149 | 1.00 | 125.93 |
| 5229 | CD | GLU D | 82 | 52.264 | 50.938 | 34.661 | 1.00 | 125.93 |
| 5230 | OE1 | GLU D | 82 | 52.389 | 50.771 | 35.897 | 1.00 | 125.93 |
| 5231 | OE2 | GLU D | 82 | 52.299 | 50.001 | 33.829 | 1.00 | 125.93 |
| 5232 | C | GLU D | 82 | 49.959 | 54.695 | 33.498 | 1.00 | 81.20 |
| 5233 | O | GLU D | 82 | 48.765 | 54.633 | 33.821 | 1.00 | 81.20 |
| 5234 | N | VAL D | 83 | 50.348 | 54.809 | 32.230 | 1.00 | 74.09 |
| 5235 | CA | VAL D | 83 | 49.379 | 54.891 | 31.140 | 1.00 | 74.09 |
| 5236 | CB | VAL D | 83 | 49.747 | 56.013 | 30.177 | 1.00 | 86.03 |
| 5237 | CG1 | VAL D | 83 | 48.810 | 55.997 | 28.998 | 1.00 | 86.03 |
| 5238 | CG2 | VAL D | 83 | 49.675 | 57.340 | 30.895 | 1.00 | 86.03 |
| 5239 | C | VAL D | 83 | 49.250 | 53.603 | 30.340 | 1.00 | 74.09 |
| 5240 | O | VAL D | 83 | 50.237 | 53.000 | 29.949 | 1.00 | 74.09 |
| 5241 | N | PHE D | 84 | 48.023 | 53.191 | 30.067 | 1.00 | 81.44 |
| 5242 | CA | PHE D | 84 | 47.811 | 51.957 | 29.331 | 1.00 | 81.44 |
| 5243 | CB | PHE D | 84 | 47.087 | 50.944 | 30.191 | 1.00 | 68.59 |
| 5244 | CG | PHE D | 84 | 47.803 | 50.598 | 31.437 | 1.00 | 68.59 |
| 5245 | CD1 | PHE D | 84 | 47.835 | 51.485 | 32.486 | 1.00 | 68.59 |
| 5246 | CD2 | PHE D | 84 | 48.418 | 49.359 | 31.583 | 1.00 | 68.59 |
| 5247 | CE1 | PHE D | 84 | 48.470 | 51.153 | 33.667 | 1.00 | 68.59 |
| 5248 | CE2 | PHE D | 84 | 49.058 | 49.016 | 32.765 | 1.00 | 68.59 |
| 5249 | CZ | PHE D | 84 | 49.078 | 49.917 | 33.809 | 1.00 | 68.59 |
| 5250 | C | PHE D | 84 | 47.029 | 52.029 | 28.041 | 1.00 | 81.44 |
| 5251 | O | PHE D | 84 | 46.324 | 52.998 | 27.746 | 1.00 | 81.44 |
| 5252 | N | SER D | 85 | 47.149 | 50.938 | 27.301 | 1.00 | 99.54 |
| 5253 | CA | SER D | 85 | 46.462 | 50.731 | 26.049 | 1.00 | 99.54 |
| 5254 | CB | SER D | 85 | 47.414 | 50.901 | 24.866 | 1.00 | 104.48 |
| 5255 | OG | SER D | 85 | 46.741 | 50.652 | 23.644 | 1.00 | 104.48 |
| 5256 | C | SER D | 85 | 46.015 | 49.277 | 26.155 | 1.00 | 99.54 |
| 5257 | O | SER D | 85 | 46.843 | 48.362 | 26.130 | 1.00 | 99.54 |
| 5258 | N | ASP D | 86 | 44.713 | 49.067 | 26.315 | 1.00 | 64.12 |
| 5259 | CA | ASP D | 86 | 44.166 | 47.724 | 26.426 | 1.00 | 64.12 |
| 5260 | CB | ASP D | 86 | 44.715 | 47.030 | 27.676 | 1.00 | 91.49 |
| 5261 | CG | ASP D | 86 | 44.939 | 45.556 | 27.454 | 1.00 | 91.49 |
| 5262 | OD1 | ASP D | 86 | 43.981 | 44.874 | 27.027 | 1.00 | 91.49 |
| 5263 | OD2 | ASP D | 86 | 46.065 | 45.077 | 27.697 | 1.00 | 91.49 |
| 5264 | C | ASP D | 86 | 42.631 | 47.816 | 26.481 | 1.00 | 64.12 |
| 5265 | O | ASP D | 86 | 42.085 | 48.907 | 26.673 | 1.00 | 64.12 |
| 5266 | N | TRP D | 87 | 41.937 | 46.686 | 26.293 | 1.00 | 76.92 |
| 5267 | CA | TRP D | 87 | 40.470 | 46.689 | 26.321 | 1.00 | 76.92 |
| 5268 | CB | TRP D | 87 | 39.893 | 45.330 | 25.950 | 1.00 | 235.26 |
| 5269 | CG | TRP D | 87 | 39.745 | 45.196 | 24.519 | 1.00 | 235.26 |
| 5270 | CD2 | TRP D | 87 | 40.716 | 44.672 | 23.629 | 1.00 | 235.26 |
| 5271 | CE2 | TRP D | 87 | 40.219 | 44.865 | 22.334 | 1.00 | 235.26 |
| 5272 | CE3 | TRP D | 87 | 41.945 | 44.025 | 23.805 | 1.00 | 235.26 |
| 5273 | CD1 | TRP D | 87 | 38.730 | 45.693 | 23.748 | 1.00 | 235.26 |
| 5274 | NE1 | TRP D | 87 | 39.014 | 45.491 | 22.409 | 1.00 | 235.26 |
| 5275 | CZ2 | TRP D | 87 | 40.942 | 44.480 | 21.244 | 1.00 | 235.26 |
| 5276 | CZ3 | TRP D | 87 | 42.651 | 43.619 | 22.683 | 1.00 | 235.26 |
| 5277 | CH2 | TRP D | 87 | 42.147 | 43.865 | 21.422 | 1.00 | 235.26 |
| 5278 | C | TRP D | 87 | 39.956 | 47.074 | 27.680 | 1.00 | 76.92 |
| 5279 | O | TRP D | 87 | 39.124 | 47.968 | 27.818 | 1.00 | 76.92 |
| 5280 | N | LEU D | 88 | 40.465 | 46.386 | 28.690 | 1.00 | 86.84 |
| 5281 | CA | LEU D | 88 | 40.070 | 46.643 | 30.064 | 1.00 | 86.84 |
| 5282 | CB | LEU D | 88 | 39.344 | 45.435 | 30.635 | 1.00 | 73.66 |
| 5283 | CG | LEU D | 88 | 38.028 | 45.109 | 29.953 | 1.00 | 73.66 |
| 5284 | CD1 | LEU D | 88 | 37.368 | 43.952 | 30.664 | 1.00 | 73.66 |
| 5285 | CD2 | LEU D | 88 | 37.156 | 46.335 | 29.991 | 1.00 | 73.66 |
| 5286 | C | LEU D | 88 | 41.248 | 46.962 | 30.953 | 1.00 | 86.84 |
| 5287 | O | LEU D | 88 | 42.330 | 46.396 | 30.820 | 1.00 | 86.84 |
| 5288 | N | LEU D | 89 | 41.022 | 47.870 | 31.883 | 1.00 | 45.19 |
| 5289 | CA | LEU D | 89 | 42.067 | 48.266 | 32.809 | 1.00 | 45.19 |
| 5290 | CB | LEU D | 89 | 42.573 | 49.655 | 32.473 | 1.00 | 158.38 |
| 5291 | CG | LEU D | 89 | 43.628 | 50.105 | 33.471 | 1.00 | 158.38 |
| 5292 | CD1 | LEU D | 89 | 44.671 | 49.002 | 33.642 | 1.00 | 158.38 |
| 5293 | CD2 | LEU D | 89 | 44.255 | 51.392 | 32.982 | 1.00 | 158.38 |
| 5294 | C | LEU D | 89 | 41.502 | 48.263 | 34.219 | 1.00 | 45.19 |
| 5295 | O | LEU D | 89 | 40.455 | 48.848 | 34.463 | 1.00 | 45.19 |
| 5296 | N | LEU D | 90 | 42.164 | 47.592 | 35.153 | 1.00 | 80.53 |
| 5297 | CA | LEU D | 90 | 41.666 | 47.579 | 36.523 | 1.00 | 80.53 |
| 5298 | CB | LEU D | 90 | 42.086 | 46.305 | 37.234 | 1.00 | 38.85 |
| 5299 | CG | LEU D | 90 | 41.710 | 46.256 | 38.724 | 1.00 | 38.85 |
| 5300 | CD1 | LEU D | 90 | 40.189 | 46.295 | 38.793 | 1.00 | 38.85 |
| 5301 | CD2 | LEU D | 90 | 42.228 | 45.002 | 39.432 | 1.00 | 38.85 |
| 5302 | C | LEU D | 90 | 42.245 | 48.766 | 37.280 | 1.00 | 80.53 |
| 5303 | O | LEU D | 90 | 43.445 | 48.858 | 37.467 | 1.00 | 80.53 |
| 5304 | N | GLN D | 91 | 41.400 | 49.670 | 37.742 | 1.00 | 44.32 |
| 5305 | CA | GLN D | 91 | 41.899 | 50.833 | 38.464 | 1.00 | 44.32 |
| 5306 | CB | GLN D | 91 | 41.209 | 52.089 | 37.953 | 1.00 | 57.44 |
| 5307 | CG | GLN D | 91 | 41.391 | 52.283 | 36.487 | 1.00 | 57.44 |
| 5308 | CD | GLN D | 91 | 40.897 | 53.611 | 36.016 | 1.00 | 57.44 |
| 5309 | OE1 | GLN D | 91 | 39.700 | 53.857 | 35.979 | 1.00 | 57.44 |
| 5310 | NE2 | GLN D | 91 | 41.816 | 54.489 | 35.664 | 1.00 | 57.44 |
| 5311 | C | GLN D | 91 | 41.685 | 50.714 | 39.963 | 1.00 | 44.32 |
| 5312 | O | GLN D | 91 | 40.691 | 50.176 | 40.435 | 1.00 | 44.32 |
| 5313 | N | ALA D | 92 | 42.613 | 51.230 | 40.737 | 1.00 | 48.50 |
| 5314 | CA | ALA D | 92 | 42.451 | 51.152 | 42.169 | 1.00 | 48.50 |
| 5315 | CB | ALA D | 92 | 43.463 | 50.199 | 42.739 | 1.00 | 52.70 |
| 5316 | C | ALA D | 92 | 42.636 | 52.538 | 42.787 | 1.00 | 48.50 |
| 5317 | O | ALA D | 92 | 43.475 | 53.347 | 42.341 | 1.00 | 48.50 |
| 5318 | N | SER D | 93 | 41.846 | 52.825 | 43.811 | 1.00 | 53.99 |
| 5319 | CA | SER D | 93 | 41.960 | 54.102 | 44.481 | 1.00 | 53.99 |
| 5320 | CB | SER D | 93 | 41.048 | 54.158 | 45.713 | 1.00 | 83.49 |
| 5321 | OG | SER D | 93 | 41.207 | 53.030 | 46.543 | 1.00 | 83.49 |
| 5322 | C | SER D | 93 | 43.412 | 54.212 | 44.877 | 1.00 | 53.99 |
| 5323 | O | SER D | 93 | 44.134 | 55.046 | 44.361 | 1.00 | 53.99 |
| 5324 | N | ALA D | 94 | 43.850 | 53.338 | 45.764 | 1.00 | 62.76 |
| 5325 | CA | ALA D | 94 | 45.232 | 53.342 | 46.220 | 1.00 | 62.76 |
| 5326 | CB | ALA D | 94 | 45.301 | 53.851 | 47.636 | 1.00 | 112.27 |
| 5327 | C | ALA D | 94 | 45.723 | 51.909 | 46.150 | 1.00 | 62.76 |
| 5328 | O | ALA D | 94 | 44.942 | 50.990 | 46.361 | 1.00 | 62.76 |
| 5329 | N | GLU D | 95 | 47.006 | 51.704 | 45.854 | 1.00 | 73.31 |
| 5330 | CA | GLU D | 95 | 47.535 | 50.339 | 45.746 | 1.00 | 73.31 |
| 5331 | CB | GLU D | 95 | 48.677 | 50.301 | 44.746 | 1.00 | 116.96 |
| 5332 | CG | GLU D | 95 | 48.262 | 50.756 | 43.364 | 1.00 | 116.96 |
| 5333 | CD | GLU D | 95 | 49.287 | 50.405 | 42.301 | 1.00 | 116.96 |
| 5334 | OE1 | GLU D | 95 | 49.057 | 50.758 | 41.121 | 1.00 | 116.96 |
| 5335 | OE2 | GLU D | 95 | 50.320 | 49.776 | 42.643 | 1.00 | 116.96 |
| 5336 | C | GLU D | 95 | 47.987 | 49.724 | 47.063 | 1.00 | 73.31 |
| 5337 | O | GLU D | 95 | 48.194 | 48.517 | 47.143 | 1.00 | 73.31 |
| 5338 | N | VAL D | 96 | 48.139 | 50.563 | 48.089 | 1.00 | 71.30 |
| 5339 | CA | VAL D | 96 | 48.557 | 50.126 | 49.422 | 1.00 | 71.30 |
| 5340 | CB | VAL D | 96 | 50.010 | 50.433 | 49.657 | 1.00 | 83.19 |
| 5341 | CG1 | VAL D | 96 | 50.502 | 49.611 | 50.812 | 1.00 | 83.19 |
| 5342 | CG2 | VAL D | 96 | 50.802 | 50.132 | 48.410 | 1.00 | 83.19 |
| 5343 | C | VAL D | 96 | 47.713 | 50.869 | 50.435 | 1.00 | 71.30 |
| 5344 | O | VAL D | 96 | 47.560 | 52.071 | 50.347 | 1.00 | 71.30 |
| 5345 | N | VAL D | 97 | 47.190 | 50.159 | 51.420 | 1.00 | 69.41 |
| 5346 | CA | VAL D | 97 | 46.277 | 50.778 | 52.365 | 1.00 | 69.41 |
| 5347 | CB | VAL D | 97 | 44.849 | 50.417 | 51.970 | 1.00 | 60.29 |
| 5348 | CG1 | VAL D | 97 | 43.889 | 51.256 | 52.717 | 1.00 | 60.29 |
| 5349 | CG2 | VAL D | 97 | 44.654 | 50.562 | 50.501 | 1.00 | 60.29 |
| 5350 | C | VAL D | 97 | 46.410 | 50.374 | 53.828 | 1.00 | 69.41 |
| 5351 | O | VAL D | 97 | 46.540 | 49.185 | 54.136 | 1.00 | 69.41 |
| 5352 | N | MET D | 98 | 46.316 | 51.350 | 54.730 | 1.00 | 72.66 |
| 5353 | CA | MET D | 98 | 46.389 | 51.084 | 56.169 | 1.00 | 72.66 |
| 5354 | CB | MET D | 98 | 46.498 | 52.404 | 56.921 | 1.00 | 249.19 |
| 5355 | CG | MET D | 98 | 47.751 | 53.177 | 56.594 | 1.00 | 249.19 |
| 5356 | SD | MET D | 98 | 49.140 | 52.518 | 57.501 | 1.00 | 249.19 |
| 5357 | CE | MET D | 98 | 48.761 | 53.180 | 59.122 | 1.00 | 249.19 |
| 5358 | C | MET D | 98 | 45.110 | 50.363 | 56.592 | 1.00 | 72.66 |
| 5359 | O | MET D | 98 | 44.014 | 50.780 | 56.201 | 1.00 | 72.66 |
| 5360 | N | GLU D | 99 | 45.234 | 49.288 | 57.373 | 1.00 | 68.49 |
| 5361 | CA | GLU D | 99 | 44.063 | 48.535 | 57.828 | 1.00 | 68.49 |
| 5362 | CB | GLU D | 99 | 44.441 | 47.605 | 58.977 | 1.00 | 249.24 |
| 5363 | CG | GLU D | 99 | 43.474 | 46.454 | 59.176 | 1.00 | 249.24 |
| 5364 | CD | GLU D | 99 | 43.683 | 45.744 | 60.499 | 1.00 | 249.24 |
| 5365 | OE1 | GLU D | 99 | 44.852 | 45.590 | 60.913 | 1.00 | 249.24 |
| 5366 | OE2 | GLU D | 99 | 42.679 | 45.331 | 61.120 | 1.00 | 249.24 |
| 5367 | C | GLU D | 99 | 43.007 | 49.529 | 58.315 | 1.00 | 68.49 |
| 5368 | O | GLU D | 99 | 43.308 | 50.396 | 59.129 | 1.00 | 68.49 |
| 5369 | N | GLY D | 100 | 41.786 | 49.439 | 57.807 | 1.00 | 99.19 |
| 5370 | CA | GLY D | 100 | 40.757 | 50.360 | 58.251 | 1.00 | 99.19 |
| 5371 | C | GLY D | 100 | 40.336 | 51.428 | 57.256 | 1.00 | 99.19 |
| 5372 | O | GLY D | 100 | 39.252 | 52.016 | 57.398 | 1.00 | 99.19 |
| 5373 | N | GLN D | 101 | 41.167 | 51.678 | 56.244 | 1.00 | 64.03 |
| 5374 | CA | GLN D | 101 | 40.845 | 52.709 | 55.249 | 1.00 | 64.03 |
| 5375 | CB | GLN D | 101 | 42.121 | 53.294 | 54.653 | 1.00 | 115.74 |
| 5376 | CG | GLN D | 101 | 42.956 | 54.053 | 55.650 | 1.00 | 115.74 |
| 5377 | CD | GLN D | 101 | 42.145 | 55.055 | 56.435 | 1.00 | 115.74 |
| 5378 | OE1 | GLN D | 101 | 41.427 | 54.698 | 57.365 | 1.00 | 115.74 |
| 5379 | NE2 | GLN D | 101 | 42.246 | 56.318 | 56.053 | 1.00 | 115.74 |
| 5380 | C | GLN D | 101 | 39.939 | 52.240 | 54.118 | 1.00 | 64.03 |
| 5381 | O | GLN D | 101 | 39.701 | 51.050 | 53.960 | 1.00 | 64.03 |
| 5382 | N | PRO D | 102 | 39.411 | 53.178 | 53.317 | 1.00 | 85.32 |
| 5383 | CD | PRO D | 102 | 39.527 | 54.647 | 53.374 | 1.00 | 90.00 |
| 5384 | CA | PRO D | 102 | 38.536 | 52.761 | 52.218 | 1.00 | 85.32 |
| 5385 | CB | PRO D | 102 | 37.759 | 54.032 | 51.911 | 1.00 | 90.00 |
| 5386 | CG | PRO D | 102 | 38.814 | 55.078 | 52.098 | 1.00 | 90.00 |
| 5387 | C | PRO D | 102 | 39.365 | 52.273 | 51.026 | 1.00 | 85.32 |
| 5388 | O | PRO D | 102 | 40.528 | 52.659 | 50.867 | 1.00 | 85.32 |
| 5389 | N | LEU D | 103 | 38.760 | 51.430 | 50.194 | 1.00 | 84.38 |
| 5390 | CA | LEU D | 103 | 39.424 | 50.903 | 49.016 | 1.00 | 84.38 |
| 5391 | CB | LEU D | 103 | 39.973 | 49.525 | 49.315 | 1.00 | 75.67 |
| 5392 | CG | LEU D | 103 | 40.655 | 48.977 | 48.070 | 1.00 | 75.67 |
| 5393 | CD1 | LEU D | 103 | 41.849 | 49.845 | 47.739 | 1.00 | 75.67 |
| 5394 | CD2 | LEU D | 103 | 41.095 | 47.543 | 48.305 | 1.00 | 75.67 |
| 5395 | C | LEU D | 103 | 38.467 | 50.792 | 47.854 | 1.00 | 84.38 |
| 5396 | O | LEU D | 103 | 37.453 | 50.135 | 47.974 | 1.00 | 84.38 |
| 5397 | N | PHE D | 104 | 38.771 | 51.419 | 46.728 | 1.00 | 75.73 |
| 5398 | CA | PHE D | 104 | 37.865 | 51.312 | 45.586 | 1.00 | 75.73 |
| 5399 | CB | PHE D | 104 | 37.272 | 52.679 | 45.208 | 1.00 | 163.52 |
| 5400 | CG | PHE D | 104 | 36.530 | 53.359 | 46.322 | 1.00 | 163.52 |
| 5401 | CD1 | PHE D | 104 | 37.222 | 53.984 | 47.352 | 1.00 | 163.52 |
| 5402 | CD2 | PHE D | 104 | 35.139 | 53.381 | 46.342 | 1.00 | 163.52 |
| 5403 | CE1 | PHE D | 104 | 36.542 | 54.625 | 48.393 | 1.00 | 163.52 |
| 5404 | CE2 | PHE D | 104 | 34.446 | 54.020 | 47.381 | 1.00 | 163.52 |
| 5405 | CZ | PHE D | 104 | 35.152 | 54.643 | 48.407 | 1.00 | 163.52 |
| 5406 | C | PHE D | 104 | 38.550 | 50.717 | 44.353 | 1.00 | 75.73 |
| 5407 | O | PHE D | 104 | 39.617 | 51.181 | 43.942 | 1.00 | 75.73 |
| 5408 | N | LEU D | 105 | 37.950 | 49.684 | 43.769 | 1.00 | 46.40 |
| 5409 | CA | LEU D | 105 | 38.504 | 49.069 | 42.561 | 1.00 | 46.40 |
| 5410 | CB | LEU D | 105 | 38.633 | 47.555 | 42.722 | 1.00 | 51.89 |
| 5411 | CG | LEU D | 105 | 39.461 | 47.169 | 43.932 | 1.00 | 51.89 |
| 5412 | CD1 | LEU D | 105 | 39.723 | 45.660 | 43.969 | 1.00 | 51.89 |
| 5413 | CD2 | LEU D | 105 | 40.750 | 47.942 | 43.836 | 1.00 | 51.89 |
| 5414 | C | LEU D | 105 | 37.518 | 49.366 | 41.456 | 1.00 | 46.40 |
| 5415 | O | LEU D | 105 | 36.330 | 49.413 | 41.701 | 1.00 | 46.40 |
| 5416 | N | ARG D | 106 | 37.988 | 49.551 | 40.236 | 1.00 | 68.20 |
| 5417 | CA | ARG D | 106 | 37.073 | 49.852 | 39.159 | 1.00 | 68.20 |
| 5418 | CB | ARG D | 106 | 37.090 | 51.354 | 38.922 | 1.00 | 103.77 |
| 5419 | CG | ARG D | 106 | 36.259 | 51.801 | 37.762 | 1.00 | 103.77 |
| 5420 | CD | ARG D | 106 | 36.514 | 53.271 | 37.452 | 1.00 | 103.77 |
| 5421 | NE | ARG D | 106 | 35.766 | 53.701 | 36.275 | 1.00 | 103.77 |
| 5422 | CZ | ARG D | 106 | 36.095 | 54.738 | 35.519 | 1.00 | 103.77 |
| 5423 | NH1 | ARG D | 106 | 37.170 | 55.458 | 35.811 | 1.00 | 103.77 |
| 5424 | NH2 | ARG D | 106 | 35.353 | 55.044 | 34.462 | 1.00 | 103.77 |
| 5425 | C | ARG D | 106 | 37.457 | 49.119 | 37.876 | 1.00 | 68.20 |
| 5426 | O | ARG D | 106 | 38.595 | 49.240 | 37.415 | 1.00 | 68.20 |
| 5427 | N | CYS D | 107 | 36.535 | 48.340 | 37.309 | 1.00 | 54.86 |
| 5428 | CA | CYS D | 107 | 36.842 | 47.659 | 36.053 | 1.00 | 54.86 |
| 5429 | C | CYS D | 107 | 36.528 | 48.688 | 34.983 | 1.00 | 54.86 |
| 5430 | O | CYS D | 107 | 35.365 | 49.000 | 34.720 | 1.00 | 54.86 |
| 5431 | CB | CYS D | 107 | 35.984 | 46.421 | 35.850 | 1.00 | 81.59 |
| 5432 | SG | CYS D | 107 | 36.664 | 45.289 | 34.601 | 1.00 | 81.59 |
| 5433 | N | HIS D | 108 | 37.578 | 49.236 | 34.384 | 1.00 | 77.64 |
| 5434 | CA | HIS D | 108 | 37.449 | 50.285 | 33.386 | 1.00 | 77.64 |
| 5435 | CB | HIS D | 108 | 38.460 | 51.352 | 33.687 | 1.00 | 84.93 |
| 5436 | CG | HIS D | 108 | 38.301 | 52.573 | 32.853 | 1.00 | 84.93 |
| 5437 | CD2 | HIS D | 108 | 39.176 | 53.230 | 32.060 | 1.00 | 84.93 |
| 5438 | ND1 | HIS D | 108 | 37.136 | 53.301 | 32.834 | 1.00 | 84.93 |
| 5439 | CE1 | HIS D | 108 | 37.306 | 54.364 | 32.068 | 1.00 | 84.93 |
| 5440 | NE2 | HIS D | 108 | 38.534 | 54.346 | 31.587 | 1.00 | 84.93 |
| 5441 | C | HIS D | 108 | 37.608 | 49.848 | 31.945 | 1.00 | 77.64 |
| 5442 | O | HIS D | 108 | 38.604 | 49.229 | 31.559 | 1.00 | 77.64 |
| 5443 | N | GLY D | 109 | 36.618 | 50.206 | 31.143 | 1.00 | 64.08 |
| 5444 | CA | GLY D | 109 | 36.637 | 49.820 | 29.750 | 1.00 | 64.08 |
| 5445 | C | GLY D | 109 | 37.367 | 50.854 | 28.945 | 1.00 | 64.08 |
| 5446 | O | GLY D | 109 | 37.498 | 52.002 | 29.379 | 1.00 | 64.08 |
| 5447 | N | TRP D | 110 | 37.858 | 50.446 | 27.781 | 1.00 | 110.56 |
| 5448 | CA | TRP D | 110 | 38.575 | 51.353 | 26.906 | 1.00 | 110.56 |
| 5449 | CB | TRP D | 110 | 39.206 | 50.578 | 25.749 | 1.00 | 129.78 |
| 5450 | CG | TRP D | 110 | 39.819 | 51.456 | 24.721 | 1.00 | 129.78 |
| 5451 | CD2 | TRP D | 110 | 41.184 | 51.879 | 24.659 | 1.00 | 129.78 |
| 5452 | CE2 | TRP D | 110 | 41.307 | 52.743 | 23.557 | 1.00 | 129.78 |
| 5453 | CE3 | TRP D | 110 | 42.322 | 51.608 | 25.434 | 1.00 | 129.78 |
| 5454 | CD1 | TRP D | 110 | 39.184 | 52.062 | 23.682 | 1.00 | 129.78 |
| 5455 | NE1 | TRP D | 110 | 40.068 | 52.836 | 22.977 | 1.00 | 129.78 |
| 5456 | CZ2 | TRP D | 110 | 42.514 | 53.345 | 23.204 | 1.00 | 129.78 |
| 5457 | CZ3 | TRP D | 110 | 43.525 | 52.208 | 25.083 | 1.00 | 129.78 |
| 5458 | CH2 | TRP D | 110 | 43.609 | 53.068 | 23.980 | 1.00 | 129.78 |
| 5459 | C | TRP D | 110 | 37.623 | 52.414 | 26.377 | 1.00 | 110.56 |
| 5460 | O | TRP D | 110 | 36.417 | 52.183 | 26.252 | 1.00 | 110.56 |
| 5461 | N | ARG D | 111 | 38.170 | 53.591 | 26.091 | 1.00 | 110.12 |
| 5462 | CA | ARG D | 111 | 37.377 | 54.696 | 25.564 | 1.00 | 110.12 |
| 5463 | CB | ARG D | 111 | 37.068 | 54.455 | 24.113 | 1.00 | 249.23 |
| 5464 | CG | ARG D | 111 | 38.127 | 54.981 | 23.233 | 1.00 | 249.23 |
| 5465 | CD | ARG D | 111 | 37.639 | 54.963 | 21.844 | 1.00 | 249.23 |
| 5466 | NE | ARG D | 111 | 38.039 | 56.180 | 21.160 | 1.00 | 249.23 |
| 5467 | CZ | ARG D | 111 | 37.564 | 57.390 | 21.444 | 1.00 | 249.23 |
| 5468 | NH1 | ARG D | 111 | 36.661 | 57.561 | 22.411 | 1.00 | 249.23 |
| 5469 | NH2 | ARG D | 111 | 38.007 | 58.437 | 20.760 | 1.00 | 249.23 |
| 5470 | C | ARG D | 111 | 36.070 | 54.939 | 26.286 | 1.00 | 110.12 |
| 5471 | O | ARG D | 111 | 35.117 | 55.496 | 25.736 | 1.00 | 110.12 |
| 5472 | N | ASN D | 112 | 36.031 | 54.502 | 27.527 | 1.00 | 80.55 |
| 5473 | CA | ASN D | 112 | 34.859 | 54.663 | 28.349 | 1.00 | 80.55 |
| 5474 | CB | ASN D | 112 | 34.546 | 56.137 | 28.546 | 1.00 | 69.20 |
| 5475 | CG | ASN D | 112 | 33.765 | 56.379 | 29.815 | 1.00 | 69.20 |
| 5476 | OD1 | ASN D | 112 | 33.075 | 55.484 | 30.307 | 1.00 | 69.20 |
| 5477 | ND2 | ASN D | 112 | 33.863 | 57.586 | 30.355 | 1.00 | 69.20 |
| 5478 | C | ASN D | 112 | 33.621 | 53.963 | 27.813 | 1.00 | 80.55 |
| 5479 | O | ASN D | 112 | 32.500 | 54.357 | 28.143 | 1.00 | 80.55 |
| 5480 | N | TRP D | 113 | 33.804 | 52.930 | 26.998 | 1.00 | 104.63 |
| 5481 | CA | TRP D | 113 | 32.649 | 52.207 | 26.504 | 1.00 | 104.63 |
| 5482 | CB | TRP D | 113 | 33.045 | 51.128 | 25.519 | 1.00 | 141.29 |
| 5483 | CG | TRP D | 113 | 33.355 | 51.652 | 24.198 | 1.00 | 141.29 |
| 5484 | CD2 | TRP D | 113 | 34.368 | 51.180 | 23.311 | 1.00 | 141.29 |
| 5485 | CE2 | TRP D | 113 | 34.278 | 51.944 | 22.133 | 1.00 | 141.29 |
| 5486 | CE3 | TRP D | 113 | 35.343 | 50.182 | 23.397 | 1.00 | 141.29 |
| 5487 | CD1 | TRP D | 113 | 32.705 | 52.655 | 23.541 | 1.00 | 141.29 |
| 5488 | NE1 | TRP D | 113 | 33.254 | 52.837 | 22.296 | 1.00 | 141.29 |
| 5489 | CZ2 | TRP D | 113 | 35.126 | 51.743 | 21.057 | 1.00 | 141.29 |
| 5490 | CZ3 | TRP D | 113 | 36.188 | 49.984 | 22.324 | 1.00 | 141.29 |
| 5491 | CH2 | TRP D | 113 | 36.075 | 50.761 | 21.173 | 1.00 | 141.29 |
| 5492 | C | TRP D | 113 | 31.928 | 51.542 | 27.656 | 1.00 | 104.63 |
| 5493 | O | TRP D | 113 | 32.215 | 51.806 | 28.828 | 1.00 | 104.63 |
| 5494 | N | ASP D | 114 | 30.990 | 50.668 | 27.313 | 1.00 | 117.64 |
| 5495 | CA | ASP D | 114 | 30.229 | 49.960 | 28.320 | 1.00 | 117.64 |
| 5496 | CB | ASP D | 114 | 28.725 | 50.109 | 28.065 | 1.00 | 192.42 |
| 5497 | CG | ASP D | 114 | 28.176 | 51.431 | 28.576 | 1.00 | 192.42 |
| 5498 | OD1 | ASP D | 114 | 28.288 | 51.685 | 29.796 | 1.00 | 192.42 |
| 5499 | OD2 | ASP D | 114 | 27.636 | 52.214 | 27.764 | 1.00 | 192.42 |
| 5500 | C | ASP D | 114 | 30.619 | 48.498 | 28.345 | 1.00 | 117.64 |
| 5501 | O | ASP D | 114 | 30.831 | 47.875 | 27.301 | 1.00 | 117.64 |
| 5502 | N | VAL D | 115 | 30.730 | 47.967 | 29.559 | 1.00 | 73.71 |
| 5503 | CA | VAL D | 115 | 31.084 | 46.577 | 29.766 | 1.00 | 73.71 |
| 5504 | CB | VAL D | 115 | 32.340 | 46.448 | 30.614 | 1.00 | 75.80 |
| 5505 | CG1 | VAL D | 115 | 32.827 | 45.011 | 30.593 | 1.00 | 75.80 |
| 5506 | CG2 | VAL D | 115 | 33.403 | 47.378 | 30.086 | 1.00 | 75.80 |
| 5507 | C | VAL D | 115 | 29.947 | 45.862 | 30.481 | 1.00 | 73.71 |
| 5508 | O | VAL D | 115 | 29.301 | 46.431 | 31.368 | 1.00 | 73.71 |
| 5509 | N | TYR D | 116 | 29.700 | 44.615 | 30.078 | 1.00 | 69.51 |
| 5510 | CA | TYR D | 116 | 28.642 | 43.810 | 30.672 | 1.00 | 69.51 |
| 5511 | CB | TYR D | 116 | 27.563 | 43.539 | 29.638 | 1.00 | 100.20 |
| 5512 | CG | TYR D | 116 | 26.886 | 44.780 | 29.133 | 1.00 | 100.20 |
| 5513 | CD1 | TYR D | 116 | 27.276 | 45.376 | 27.942 | 1.00 | 100.20 |
| 5514 | CE1 | TYR D | 116 | 26.660 | 46.549 | 27.481 | 1.00 | 100.20 |
| 5515 | CD2 | TYR D | 116 | 25.866 | 45.375 | 29.858 | 1.00 | 100.20 |
| 5516 | CE2 | TYR D | 116 | 25.243 | 46.545 | 29.412 | 1.00 | 100.20 |
| 5517 | CZ | TYR D | 116 | 25.648 | 47.127 | 28.225 | 1.00 | 100.20 |
| 5518 | OH | TYR D | 116 | 25.060 | 48.293 | 27.795 | 1.00 | 100.20 |
| 5519 | C | TYR D | 116 | 29.179 | 42.488 | 31.222 | 1.00 | 69.51 |
| 5520 | O | TYR D | 116 | 30.341 | 42.127 | 30.986 | 1.00 | 69.51 |
| 5521 | N | LYS D | 117 | 28.327 | 41.766 | 31.947 | 1.00 | 88.92 |
| 5522 | CA | LYS D | 117 | 28.708 | 40.492 | 32.541 | 1.00 | 88.92 |
| 5523 | CB | LYS D | 117 | 28.772 | 39.397 | 31.480 | 1.00 | 111.93 |
| 5524 | CG | LYS D | 117 | 27.453 | 38.715 | 31.180 | 1.00 | 111.93 |
| 5525 | CD | LYS D | 117 | 27.695 | 37.387 | 30.471 | 1.00 | 11.93 |
| 5526 | CE | LYS D | 117 | 28.540 | 36.435 | 31.338 | 1.00 | 111.93 |
| 5527 | NZ | LYS D | 117 | 28.852 | 35.125 | 30.675 | 1.00 | 111.93 |
| 5528 | C | LYS D | 117 | 30.069 | 40.625 | 33.213 | 1.00 | 88.92 |
| 5529 | O | LYS D | 117 | 31.002 | 39.882 | 32.909 | 1.00 | 88.92 |
| 5530 | N | VAL D | 118 | 30.182 | 41.578 | 34.129 | 1.00 | 81.88 |
| 5531 | CA | VAL D | 118 | 32.433 | 41.816 | 34.828 | 1.00 | 81.88 |
| 5532 | CB | VAL D | 118 | 31.524 | 43.274 | 35.241 | 1.00 | 84.78 |
| 5533 | CG1 | VAL D | 118 | 32.404 | 43.434 | 36.459 | 1.00 | 84.78 |
| 5534 | CG2 | VAL D | 118 | 32.104 | 44.055 | 34.101 | 1.00 | 84.78 |
| 5535 | C | VAL D | 118 | 31.693 | 40.949 | 36.052 | 1.00 | 81.88 |
| 5536 | O | VAL D | 118 | 30.803 | 40.742 | 36.893 | 1.00 | 81.88 |
| 5537 | N | ILE D | 119 | 32.928 | 40.468 | 36.171 | 1.00 | 56.52 |
| 5538 | CA | ILE D | 119 | 33.296 | 39.637 | 37.310 | 1.00 | 56.52 |
| 5539 | CB | ILE D | 119 | 33.364 | 38.181 | 36.895 | 1.00 | 59.73 |
| 5540 | CG2 | ILE D | 119 | 33.652 | 37.309 | 38.094 | 1.00 | 59.73 |
| 5541 | CG1 | ILE D | 119 | 32.058 | 37.776 | 36.217 | 1.00 | 59.73 |
| 5542 | CD1 | ILE D | 119 | 32.154 | 36.446 | 35.534 | 1.00 | 59.73 |
| 5543 | C | ILE D | 119 | 34.662 | 40.027 | 37.826 | 1.00 | 56.52 |
| 5544 | O | ILE D | 119 | 35.611 | 40.026 | 37.0357 | 1.00 | 56.52 |
| 5545 | N | TYR D | 120 | 34.785 | 40.378 | 39.104 | 1.00 | 151.66 |
| 5546 | CA | TYR D | 120 | 36.115 | 40.736 | 39.618 | 1.00 | 51.66 |
| 5547 | CB | TYR D | 120 | 36.064 | 41.770 | 40.742 | 1.00 | 57.63 |
| 5548 | CG | TYR D | 120 | 35.658 | 43.139 | 40.320 | 1.00 | 57.63 |
| 5549 | CD1 | TYR D | 120 | 34.336 | 43.470 | 40.170 | 1.00 | 57.63 |
| 5550 | CE1 | TYR D | 120 | 33.960 | 44.720 | 39.744 | 1.00 | 57.63 |
| 5551 | CD2 | TYR D | 120 | 36.599 | 44.093 | 40.038 | 1.00 | 57.63 |
| 5552 | CE2 | TYR D | 120 | 36.237 | 45.353 | 39.609 | 1.00 | 57.63 |
| 5553 | CZ | TYR D | 120 | 34.915 | 45.656 | 39.464 | 1.00 | 57.63 |
| 5554 | OH | TYR D | 120 | 34.549 | 46.902 | 39.039 | 1.00 | 57.63 |
| 5555 | C | TYR D | 120 | 36.702 | 39.486 | 40.200 | 1.00 | 51.66 |
| 5556 | O | TYR D | 120 | 35.971 | 38.657 | 40.725 | 1.00 | 51.66 |
| 5557 | N | TYR D | 121 | 38.015 | 39.353 | 40.123 | 1.00 | 46.59 |
| 5558 | CA | TYR D | 121 | 38.667 | 38.180 | 40.684 | 1.00 | 46.59 |
| 5559 | CB | TYR D | 121 | 39.304 | 37.344 | 39.572 | 1.00 | 81.03 |
| 5560 | CG | TYR D | 121 | 38.357 | 36.640 | 38.623 | 1.00 | 81.03 |
| 5561 | CD1 | TYR D | 121 | 37.541 | 37.362 | 37.761 | 1.00 | 81.03 |
| 5562 | CE1 | TYR D | 121 | 36.705 | 36.721 | 36.856 | 1.00 | 81.03 |
| 5563 | CD2 | TYR D | 121 | 38.311 | 35.244 | 38.562 | 1.00 | 81.03 |
| 5564 | CE2 | TYR D | 121 | 37.478 | 34.597 | 37.666 | 1.00 | 81.03 |
| 5565 | CZ | TYR D | 121 | 36.672 | 35.345 | 36.808 | 1.00 | 81.03 |
| 5566 | OH | TYR D | 121 | 35.835 | 34.720 | 35.894 | 1.00 | 81.03 |
| 5567 | C | TYR D | 121 | 39.771 | 38.566 | 41.683 | 1.00 | 46.59 |
| 5568 | O | TYR D | 121 | 40.518 | 39.538 | 41.473 | 1.00 | 46.59 |
| 5569 | N | LYS D | 122 | 39.876 | 37.809 | 42.770 | 1.00 | 72.20 |
| 5570 | CA | LYS D | 122 | 40.920 | 38.054 | 43.759 | 1.00 | 72.20 |
| 5571 | CB | LYS D | 122 | 40.357 | 38.585 | 45.073 | 1.00 | 128.16 |
| 5572 | CG | LYS D | 122 | 41.440 | 38.842 | 46.100 | 1.00 | 128.16 |
| 5573 | CD | LYS D | 122 | 40.869 | 39.066 | 47.470 | 1.00 | 128.16 |
| 5574 | CE | LYS D | 122 | 41.973 | 39.176 | 48.496 | 1.00 | 128.16 |
| 5575 | NZ | LYS D | 122 | 41.394 | 39.233 | 49.865 | 1.00 | 128.16 |
| 5576 | C | LYS D | 122 | 41.598 | 36.736 | 44.028 | 1.00 | 72.20 |
| 5576 | O | LYS D | 122 | 40.977 | 35.813 | 44.536 | 1.00 | 72.20 |
| 5578 | N | ASP D | 123 | 42.876 | 36.658 | 43.692 | 1.00 | 101.46 |
| 5579 | CA | ASP D | 123 | 43.660 | 35.450 | 43.884 | 1.00 | 101.46 |
| 5580 | CB | ASP D | 123 | 43.802 | 35.135 | 45.375 | 1.00 | 177.22 |
| 5581 | CG | ASP D | 123 | 44.795 | 36.049 | 46.065 | 1.00 | 177.22 |
| 5582 | OD1 | ASP D | 123 | 45.903 | 36.238 | 45.518 | 1.00 | 177.22 |
| 5583 | OD2 | ASP D | 123 | 44.477 | 36.572 | 47.154 | 1.00 | 177.22 |
| 5584 | C | ASP D | 123 | 43.079 | 34.258 | 43.138 | 1.00 | 101.46 |
| 5585 | O | ASP D | 123 | 43.017 | 33.147 | 43.668 | 1.00 | 101.46 |
| 5586 | N | GLY D | 124 | 42.661 | 34.502 | 41.898 | 1.00 | 69.52 |
| 5587 | CA | GLY D | 124 | 42.103 | 33.456 | 41.056 | 1.00 | 89.52 |
| 5588 | C | GLY D | 124 | 40.673 | 33.041 | 41.346 | 1.00 | 89.52 |
| 5589 | O | GLY D | 124 | 40.092 | 32.261 | 40.587 | 1.00 | 89.52 |
| 5590 | N | GLU D | 125 | 40.097 | 33.559 | 42.428 | 1.00 | 72.85 |
| 5591 | CA | GLU D | 125 | 38.730 | 33.206 | 42.826 | 1.00 | 72.85 |
| 5592 | CB | GLU D | 125 | 38.599 | 33.194 | 44.362 | 1.00 | 232.74 |
| 5593 | CG | GLU D | 125 | 39.348 | 32.082 | 45.103 | 1.00 | 232.74 |
| 5594 | CD | GLU D | 125 | 38.625 | 30.746 | 45.068 | 1.00 | 232.74 |
| 5595 | OE1 | GLU D | 125 | 37.493 | 30.662 | 45.593 | 1.00 | 232.74 |
| 5596 | OE2 | GLU D | 125 | 39.194 | 29.780 | 44.517 | 1.00 | 232.74 |
| 5597 | C | GLU D | 125 | 37.706 | 34.202 | 42.280 | 1.00 | 72.85 |
| 5598 | O | GLU D | 125 | 37.974 | 35.404 | 42.183 | 1.00 | 72.85 |
| 5599 | N | ALA D | 126 | 36.527 | 33.708 | 41.926 | 1.00 | 95.10 |
| 5600 | CA | ALA D | 126 | 35.472 | 34.595 | 41.450 | 1.00 | 95.10 |
| 5601 | CB | ALA D | 126 | 34.290 | 33.791 | 40.991 | 1.00 | 132.03 |
| 5602 | C | ALA D | 126 | 35.119 | 35.403 | 42.693 | 1.00 | 95.10 |
| 5603 | O | ALA D | 126 | 35.153 | 34.869 | 43.802 | 1.00 | 95.10 |
| 5604 | N | LEU D | 127 | 34.782 | 36.678 | 42.531 | 1.00 | 64.20 |
| 5605 | CA | LEU D | 127 | 34.470 | 37.522 | 43.697 | 1.00 | 64.20 |
| 5606 | CB | LEU D | 127 | 35.559 | 38.566 | 43.919 | 1.00 | 89.10 |
| 5607 | CG | LEU D | 127 | 35.546 | 38.957 | 45.392 | 1.00 | 89.10 |
| 5608 | CD1 | LEU D | 127 | 35.768 | 37.691 | 46.219 | 1.00 | 89.10 |
| 5609 | CD2 | LEU D | 127 | 36.612 | 39.988 | 45.686 | 1.00 | 89.10 |
| 5610 | C | LEU D | 127 | 33.138 | 38.237 | 43.722 | 1.00 | 64.20 |
| 5611 | O | LEU D | 127 | 32.408 | 38.098 | 44.686 | 1.00 | 64.20 |
| 5612 | N | LYS D | 128 | 32.859 | 39.041 | 42.702 | 1.00 | 63.83 |
| 5613 | CA | LYS D | 128 | 31.584 | 39.742 | 42.583 | 1.00 | 63.83 |
| 5614 | CB | LYS D | 128 | 31.737 | 41.203 | 43.000 | 1.00 | 126.01 |
| 5615 | CG | LYS D | 128 | 32.165 | 41.409 | 44.431 | 1.00 | 126.01 |
| 5616 | CD | LYS D | 128 | 31.058 | 41.080 | 45.416 | 1.00 | 126.01 |
| 5617 | CE | LYS D | 128 | 31.491 | 41.410 | 46.843 | 1.00 | 126.01 |
| 5618 | NZ | LYS D | 128 | 30.404 | 41.252 | 47.855 | 1.00 | 126.01 |
| 5619 | C | LYS D | 128 | 31.160 | 39.675 | 41.109 | 1.00 | 63.83 |
| 5620 | O | LYS D | 128 | 32.021 | 39.580 | 40.219 | 1.00 | 63.83 |
| 5621 | N | TYR D | 129 | 29.857 | 39.741 | 40.833 | 1.00 | 62.64 |
| 5622 | CA | TYR D | 129 | 29.387 | 39.683 | 39.444 | 1.00 | 62.64 |
| 5623 | CB | TYR D | 129 | 28.984 | 38.268 | 39.098 | 1.00 | 80.75 |
| 5624 | CG | TYR D | 129 | 28.046 | 38.200 | 37.928 | 1.00 | 80.75 |
| 5625 | CD1 | TYR D | 129 | 28.521 | 38.321 | 36.629 | 1.00 | 80.75 |
| 5626 | CE1 | TYR D | 129 | 27.652 | 38.296 | 35.533 | 1.00 | 80.75 |
| 5627 | CD2 | TYR D | 129 | 26.682 | 38.057 | 38.118 | 1.00 | 80.75 |
| 5628 | CE2 | TYR D | 129 | 25.803 | 38.042 | 37.037 | 1.00 | 80.75 |
| 5629 | CZ | TYR D | 129 | 26.288 | 38.160 | 35.741 | 1.00 | 80.75 |
| 5630 | OH | TYR D | 129 | 25.412 | 38.145 | 34.662 | 1.00 | 80.75 |
| 5631 | C | TYR D | 129 | 28.192 | 40.564 | 39.182 | 1.00 | 62.64 |
| 5632 | O | TYR D | 129 | 27.268 | 40.602 | 39.996 | 1.00 | 62.64 |
| 5633 | N | TRP D | 130 | 28.190 | 41.252 | 38.042 | 1.00 | 93.45 |
| 5634 | CA | TRP D | 130 | 27.076 | 42.123 | 37.680 | 1.00 | 93.45 |
| 5635 | CB | TRP D | 130 | 27.356 | 43.561 | 38.092 | 1.00 | 113.53 |
| 5636 | CG | TRP D | 130 | 27.799 | 43.749 | 39.506 | 1.00 | 113.53 |
| 5637 | CD2 | TRP D | 130 | 27.020 | 44.284 | 40.583 | 1.00 | 113.53 |
| 5638 | CE2 | TRP D | 130 | 27.863 | 44.336 | 41.718 | 1.00 | 113.53 |
| 5639 | CE3 | TRP D | 130 | 25.690 | 44.716 | 40.700 | 1.00 | 113.53 |
| 5640 | CD1 | TRP D | 130 | 29.043 | 43.512 | 40.018 | 1.00 | 113.53 |
| 5641 | NE1 | TRP D | 130 | 29.092 | 43.864 | 41.347 | 1.00 | 113.53 |
| 5642 | CZ2 | TRP D | 130 | 27.413 | 44.811 | 42.961 | 1.00 | 113.53 |
| 5643 | CZ3 | TRP D | 130 | 25.242 | 45.191 | 41.945 | 1.00 | 113.53 |
| 5644 | CH2 | TRP D | 130 | 26.104 | 45.238 | 43.051 | 1.00 | 113.53 |
| 5645 | C | TRP D | 130 | 26.817 | 42.119 | 36.181 | 1.00 | 93.45 |
| 5646 | O | TRP D | 130 | 27.643 | 41.649 | 35.404 | 1.00 | 93.45 |
| 5647 | N | TYR D | 131 | 25.667 | 42.650 | 35.773 | 1.00 | 68.85 |
| 5648 | CA | TYR D | 131 | 25.343 | 42.732 | 34.351 | 1.00 | 68.85 |
| 5649 | CB | TYR D | 131 | 23.835 | 42.746 | 34.119 | 1.00 | 129.65 |
| 5650 | CG | TYR D | 131 | 23.515 | 42.570 | 32.657 | 1.00 | 129.65 |
| 5651 | CD1 | TYR D | 131 | 23.660 | 41.330 | 32.046 | 1.00 | 129.65 |
| 5652 | CE1 | TYR D | 131 | 23.494 | 41.183 | 30.685 | 1.00 | 129.65 |
| 5653 | CD2 | TYR D | 131 | 23.182 | 43.661 | 31.862 | 1.00 | 129.65 |
| 5654 | CE2 | TYR D | 131 | 23.015 | 43.523 | 30.495 | 1.00 | 129.65 |
| 5655 | CZ | TYR D | 131 | 23.177 | 42.282 | 29.915 | 1.00 | 129.65 |
| 5656 | OH | TYR D | 131 | 23.056 | 42.154 | 28.555 | 1.00 | 129.65 |
| 5657 | C | TYR D | 131 | 25.953 | 44.035 | 33.846 | 1.00 | 68.85 |
| 5658 | O | TYR D | 131 | 27.035 | 44.028 | 33.249 | 1.00 | 68.85 |
| 5659 | N | GLU D | 132 | 25.234 | 45.142 | 34.045 | 1.00 | 110.47 |
| 5660 | CA | GLU D | 132 | 25.761 | 46.455 | 33.684 | 1.00 | 110.47 |
| 5661 | CB | GLU D | 132 | 24.715 | 47.569 | 33.878 | 1.00 | 169.41 |
| 5662 | CG | GLU D | 132 | 23.632 | 47.688 | 32.798 | 1.00 | 169.41 |
| 5663 | CD | GLU D | 132 | 23.679 | 49.022 | 32.059 | 1.00 | 169.41 |
| 5664 | OE1 | GLU D | 132 | 24.276 | 49.981 | 32.594 | 1.00 | 169.41 |
| 5665 | OE2 | GLU D | 132 | 23.107 | 49.114 | 30.951 | 1.00 | 169.41 |
| 5666 | C | GLU D | 132 | 26.765 | 46.491 | 34.814 | 1.00 | 110.47 |
| 5667 | O | GLU D | 132 | 26.391 | 46.249 | 35.965 | 1.00 | 110.47 |
| 5668 | N | ASN D | 133 | 20.023 | 40.777 | 34.515 | 1.00 | 115.67 |
| 5669 | CA | ASN D | 133 | 29.030 | 46.736 | 35.574 | 1.00 | 115.67 |
| 5670 | CB | ASN D | 133 | 30.448 | 46.807 | 35.003 | 1.00 | 113.79 |
| 5671 | CG | ASN D | 133 | 30.911 | 48.198 | 34.814 | 1.00 | 113.79 |
| 5672 | OD1 | ASN D | 133 | 30.200 | 49.009 | 34.233 | 1.00 | 113.79 |
| 5673 | ND2 | ASN D | 133 | 32.110 | 48.501 | 35.296 | 1.00 | 113.79 |
| 5674 | C | ASN D | 133 | 28.888 | 47.723 | 36.718 | 1.00 | 115.67 |
| 5675 | O | ASN D | 133 | 28.054 | 48.618 | 36.717 | 1.00 | 115.67 |
| 5676 | N | HIS D | 134 | 29.747 | 47.518 | 37.702 | 1.00 | 133.45 |
| 5677 | CA | HIS D | 134 | 29.748 | 48.283 | 38.921 | 1.00 | 133.45 |
| 5678 | CB | HIS D | 134 | 29.100 | 47.430 | 40.006 | 1.00 | 207.58 |
| 5679 | CG | HIS D | 134 | 28.893 | 48.141 | 41.302 | 1.00 | 207.58 |
| 5680 | CD2 | HIS D | 134 | 29.411 | 47.919 | 42.534 | 1.00 | 207.58 |
| 5681 | ND1 | HIS D | 134 | 28.039 | 49.213 | 41.435 | 1.00 | 207.58 |
| 5682 | CE1 | HIS D | 134 | 28.038 | 49.620 | 42.690 | 1.00 | 207.58 |
| 5683 | NE2 | HIS D | 134 | 28.862 | 48.851 | 43.378 | 1.00 | 207.58 |
| 5684 | C | HIS D | 134 | 31.186 | 48.609 | 39.290 | 1.00 | 133.45 |
| 5685 | O | HIS D | 134 | 32.091 | 48.473 | 38.469 | 1.00 | 133.45 |
| 5686 | N | ASN D | 135 | 31.388 | 49.015 | 40.537 | 1.00 | 73.71 |
| 5687 | CA | ASN D | 135 | 32.701 | 49.394 | 41.017 | 1.00 | 73.71 |
| 5688 | CB | ASN D | 135 | 32.877 | 50.923 | 40.922 | 1.00 | 127.04 |
| 5689 | CG | ASN D | 135 | 32.913 | 51.402 | 39.490 | 1.00 | 127.04 |
| 5690 | OD1 | ASN D | 135 | 33.606 | 50.790 | 38.668 | 1.00 | 127.04 |
| 5691 | ND2 | ASN D | 135 | 32.193 | 52.478 | 39.170 | 1.00 | 127.04 |
| 5692 | C | ASN D | 135 | 32.869 | 48.930 | 42.441 | 1.00 | 73.71 |
| 5693 | O | ASN D | 135 | 32.604 | 49.672 | 43.363 | 1.00 | 73.71 |
| 5694 | N | ILE D | 136 | 33.307 | 47.689 | 42.606 | 1.00 | 72.93 |
| 5695 | CA | ILE D | 136 | 33.534 | 47.094 | 43.918 | 1.00 | 72.93 |
| 5696 | CB | ILE D | 136 | 34.435 | 45.852 | 43.786 | 1.00 | 89.30 |
| 5697 | CG2 | ILE D | 136 | 35.652 | 46.164 | 42.961 | 1.00 | 89.30 |
| 5698 | CG1 | ILE D | 136 | 34.828 | 45.348 | 45.159 | 1.00 | 89.30 |
| 5699 | CD1 | ILE D | 136 | 35.595 | 44.057 | 45.088 | 1.00 | 89.30 |
| 5700 | C | ILE D | 136 | 34.139 | 48.097 | 44.892 | 1.00 | 72.93 |
| 5701 | O | ILE D | 136 | 35.241 | 48.602 | 44.689 | 1.00 | 72.93 |
| 5702 | N | SER D | 137 | 33.393 | 48.393 | 45.952 | 1.00 | 72.75 |
| 5703 | CA | SER D | 137 | 33.810 | 49.378 | 46.952 | 1.00 | 72.75 |
| 5704 | CB | SER D | 137 | 32.797 | 50.514 | 46.982 | 1.00 | 69.16 |
| 5705 | OG | SER D | 137 | 32.966 | 51.300 | 48.135 | 1.00 | 69.16 |
| 5706 | C | SER D | 137 | 33.965 | 48.811 | 48.356 | 1.00 | 72.75 |
| 5707 | O | SER D | 137 | 33.227 | 47.922 | 48.765 | 1.00 | 72.75 |
| 5708 | N | ILE D | 138 | 34.914 | 49.348 | 49.106 | 1.00 | 112.14 |
| 5709 | CA | ILE D | 138 | 35.171 | 48.888 | 50.464 | 1.00 | 112.14 |
| 5710 | CB | ILE D | 138 | 36.436 | 48.063 | 50.515 | 1.00 | 65.89 |
| 5711 | CG2 | ILE D | 138 | 36.827 | 47.824 | 51.962 | 1.00 | 65.89 |
| 5712 | CG1 | ILE D | 138 | 36.239 | 46.759 | 49.745 | 1.00 | 65.89 |
| 5713 | CD1 | ILE D | 138 | 37.523 | 46.004 | 49.492 | 1.00 | 65.89 |
| 5714 | C | ILE D | 138 | 35.359 | 50.050 | 51.431 | 1.00 | 112.14 |
| 5715 | O | ILE D | 138 | 36.253 | 50.879 | 51.244 | 1.00 | 112.14 |
| 5716 | N | THR D | 139 | 34.544 | 50.092 | 52.483 | 1.00 | 70.90 |
| 5717 | CA | THR D | 139 | 34.628 | 51.172 | 53.464 | 1.00 | 70.90 |
| 5718 | CB | THR D | 139 | 33.330 | 51.276 | 54.253 | 1.00 | 212.07 |
| 5719 | OG1 | THR D | 139 | 32.988 | 49.984 | 54.771 | 1.00 | 212.07 |
| 5720 | CG2 | THR D | 139 | 32.212 | 51.772 | 53.352 | 1.00 | 212.07 |
| 5721 | C | THR D | 139 | 35.791 | 50.913 | 54.409 | 1.00 | 70.90 |
| 5722 | O | THR D | 139 | 36.851 | 51.523 | 54.280 | 1.00 | 70.90 |
| 5723 | N | ASN D | 140 | 35.586 | 50.005 | 55.356 | 1.00 | 80.70 |
| 5724 | CA | ASN D | 140 | 36.606 | 49.640 | 56.333 | 1.00 | 80.70 |
| 5725 | CB | ASN D | 140 | 35.957 | 49.206 | 57.644 | 1.00 | 232.63 |
| 5726 | CG | ASN D | 140 | 36.967 | 48.690 | 58.636 | 1.00 | 232.63 |
| 5727 | OD1 | ASN D | 140 | 37.818 | 47.864 | 58.318 | 1.00 | 232.63 |
| 5728 | ND2 | ASN D | 140 | 36.862 | 49.170 | 59.865 | 1.00 | 232.63 |
| 5729 | C | ASN D | 140 | 37.344 | 48.464 | 55.709 | 1.00 | 80.70 |
| 5730 | O | ASN D | 140 | 36.732 | 47.447 | 55.411 | 1.00 | 80.70 |
| 5731 | N | ALA D | 141 | 38.650 | 48.593 | 55.503 | 1.00 | 77.89 |
| 5732 | CA | ALA D | 141 | 39.428 | 47.522 | 54.870 | 1.00 | 77.89 |
| 5733 | CB | ALA D | 141 | 40.437 | 48.086 | 53.919 | 1.00 | 47.27 |
| 5734 | C | ALA D | 141 | 40.142 | 46.600 | 55.822 | 1.00 | 77.89 |
| 5735 | O | ALA D | 141 | 40.885 | 47.017 | 56.703 | 1.00 | 77.89 |
| 5736 | N | THR D | 142 | 39.941 | 45.317 | 55.606 | 1.00 | 73.90 |
| 5737 | CA | THR D | 142 | 40.557 | 44.300 | 56.434 | 1.00 | 73.90 |
| 5738 | CB | THR D | 142 | 39.702 | 43.013 | 56.412 | 1.00 | 158.80 |
| 5739 | OG1 | THR D | 142 | 38.346 | 43.343 | 56.745 | 1.00 | 158.80 |
| 5740 | CG2 | THR D | 142 | 40.214 | 42.014 | 57.416 | 1.00 | 158.80 |
| 5741 | C | THR D | 142 | 41.927 | 44.055 | 55.837 | 1.00 | 73.90 |
| 5742 | O | THR D | 142 | 42.236 | 44.606 | 54.779 | 1.00 | 73.90 |
| 5743 | N | VAL D | 143 | 42.756 | 43.268 | 56.521 | 1.00 | 104.07 |
| 5744 | CA | VAL D | 143 | 44.088 | 42.953 | 56.008 | 1.00 | 104.07 |
| 5745 | CB | VAL O | 143 | 45.093 | 42.610 | 57.127 | 1.00 | 127.52 |
| 5746 | CG1 | VAL D | 143 | 44.701 | 41.320 | 57.807 | 1.00 | 127.52 |
| 5747 | CG2 | VAL D | 143 | 46.495 | 42.479 | 56.545 | 1.00 | 127.52 |
| 5748 | C | VAL D | 143 | 43.955 | 41.736 | 55.107 | 1.00 | 104.07 |
| 5749 | O | VAL D | 143 | 44.846 | 41.424 | 54.322 | 1.00 | 104.07 |
| 5750 | N | GLU D | 144 | 42.829 | 41.046 | 55.222 | 1.00 | 87.28 |
| 5751 | CA | GLU D | 144 | 42.603 | 39.874 | 54.400 | 1.00 | 87.28 |
| 5752 | CB | GLU D | 144 | 41.492 | 39.011 | 55.003 | 1.00 | 215.80 |
| 5753 | CG | GLU D | 144 | 41.840 | 38.428 | 56.363 | 1.00 | 215.80 |
| 5754 | CD | GLU D | 144 | 40.992 | 39.001 | 57.475 | 1.00 | 215.80 |
| 5755 | OE1 | GLU D | 144 | 39.756 | 38.856 | 57.409 | 1.00 | 215.80 |
| 5756 | OE2 | GLU D | 144 | 41.555 | 39.596 | 58.416 | 1.00 | 215.80 |
| 5757 | C | GLU D | 144 | 42.245 | 40.287 | 52.982 | 1.00 | 87.28 |
| 5758 | O | GLU D | 144 | 42.288 | 39.478 | 52.074 | 1.00 | 87.28 |
| 5759 | N | ASP D | 145 | 41.898 | 41.556 | 52.801 | 1.00 | 67.03 |
| 5760 | CA | ASP D | 145 | 41.533 | 42.083 | 51.491 | 1.00 | 67.03 |
| 5761 | CB | ASP D | 145 | 40.847 | 43.441 | 51.634 | 1.00 | 129.44 |
| 5762 | CG | ASP D | 145 | 39.448 | 43.320 | 52.163 | 1.00 | 129.44 |
| 5763 | OD1 | ASP D | 145 | 38.636 | 42.636 | 51.510 | 1.00 | 129.44 |
| 5764 | OD2 | ASP D | 145 | 39.153 | 43.899 | 53.228 | 1.00 | 129.44 |
| 5765 | C | ASP D | 145 | 42.751 | 42.217 | 50.587 | 1.00 | 67.03 |
| 5766 | O | ASP D | 145 | 42.634 | 42.396 | 49.365 | 1.00 | 67.03 |
| 5767 | N | SER D | 146 | 43.932 | 42.121 | 51.177 | 1.00 | 89.85 |
| 5768 | CA | SER D | 146 | 45.140 | 42.235 | 50.383 | 1.00 | 89.85 |
| 5769 | CB | SER D | 146 | 46.366 | 42.277 | 51.300 | 1.00 | 212.33 |
| 5770 | OG | SER D | 146 | 46.317 | 43.408 | 52.152 | 1.00 | 212.33 |
| 5771 | C | SER D | 146 | 45.185 | 41.034 | 49.452 | 1.00 | 89.85 |
| 5772 | O | SER D | 146 | 44.810 | 39.933 | 49.836 | 1.00 | 89.85 |
| 5773 | N | GLY D | 147 | 45.604 | 41.256 | 48.213 | 1.00 | 67.51 |
| 5774 | CA | GLY D | 147 | 45.698 | 40.164 | 47.254 | 1.00 | 67.51 |
| 5775 | C | GLY D | 147 | 46.000 | 40.693 | 45.865 | 1.00 | 67.51 |
| 5776 | O | GLY D | 147 | 46.475 | 41.825 | 45.724 | 1.00 | 67.51 |
| 5777 | N | THR D | 148 | 45.740 | 39.888 | 44.835 | 1.00 | 62.13 |
| 5778 | CA | THR D | 148 | 45.975 | 40.327 | 43.454 | 1.00 | 62.13 |
| 5779 | CB | THR D | 148 | 47.073 | 39.493 | 42.770 | 1.00 | 85.26 |
| 5780 | OG1 | THR D | 148 | 46.483 | 38.564 | 41.871 | 1.00 | 85.26 |
| 5781 | CG2 | THR D | 148 | 47.863 | 38.729 | 43.800 | 1.00 | 85.26 |
| 5782 | C | THR D | 148 | 44.665 | 40.210 | 42.689 | 1.00 | 62.13 |
| 5783 | O | THR D | 148 | 44.106 | 39.134 | 42.527 | 1.00 | 62.13 |
| 5784 | N | TYR D | 149 | 44.164 | 41.339 | 42.230 | 1.00 | 42.52 |
| 5785 | CA | TYR D | 149 | 42.894 | 41.349 | 41.547 | 1.00 | 42.52 |
| 5786 | CB | TYR D | 149 | 42.072 | 42.518 | 42.079 | 1.00 | 42.86 |
| 5787 | CG | TYR D | 149 | 41.722 | 42.498 | 43.543 | 1.00 | 42.86 |
| 5788 | CD1 | TYR D | 149 | 42.689 | 42.662 | 44.522 | 1.00 | 42.86 |
| 5789 | CE1 | TYR D | 149 | 42.339 | 42.702 | 45.880 | 1.00 | 42.86 |
| 5790 | CD2 | TYR D | 149 | 40.404 | 42.373 | 43.936 | 1.00 | 42.86 |
| 5791 | CE2 | TYR D | 149 | 40.038 | 42.412 | 45.251 | 1.00 | 42.86 |
| 5792 | CZ | TYR D | 149 | 40.998 | 42.570 | 46.237 | 1.00 | 42.86 |
| 5793 | OH | TYR D | 149 | 40.592 | 42.542 | 47.568 | 1.00 | 42.86 |
| 5794 | C | TYR D | 149 | 43.028 | 41.506 | 40.046 | 1.00 | 42.52 |
| 5795 | O | TYR D | 149 | 44.102 | 41.847 | 39.556 | 1.00 | 42.52 |
| 5796 | N | TYR D | 150 | 41.921 | 41.262 | 39.340 | 1.00 | 57.99 |
| 5797 | CA | TYR D | 150 | 41.799 | 41.429 | 37.892 | 1.00 | 57.99 |
| 5798 | CB | TYR D | 150 | 42.675 | 40.420 | 37.108 | 1.00 | 88.00 |
| 5799 | CG | TYR D | 150 | 42.197 | 38.986 | 36.975 | 1.00 | 88.00 |
| 5800 | CD1 | TYR D | 150 | 41.124 | 38.661 | 36.156 | 1.00 | 88.00 |
| 5801 | CE1 | TYR D | 150 | 40.693 | 37.342 | 36.010 | 1.00 | 88.00 |
| 5802 | CD2 | TYR D | 150 | 42.840 | 37.948 | 37.649 | 1.00 | 88.00 |
| 5803 | CE2 | TYR D | 150 | 42.422 | 36.626 | 37.513 | 1.00 | 88.00 |
| 5804 | CZ | TYR D | 150 | 41.342 | 36.327 | 36.692 | 1.00 | 88.00 |
| 5805 | OH | TYR D | 150 | 40.898 | 35.020 | 36.578 | 1.00 | 88.00 |
| 5806 | C | TYR D | 150 | 40.293 | 41.260 | 37.623 | 1.00 | 57.99 |
| 5807 | O | TYR D | 150 | 39.569 | 40.702 | 38.470 | 1.00 | 57.99 |
| 5808 | N | CYS D | 151 | 39.793 | 41.782 | 36.500 | 1.00 | 62.97 |
| 5809 | CA | CYS D | 151 | 38.365 | 41.650 | 36.193 | 1.00 | 62.97 |
| 5810 | C | CYS D | 151 | 38.136 | 41.175 | 34.780 | 1.00 | 62.97 |
| 5811 | O | CYS D | 151 | 39.009 | 41.329 | 33.931 | 1.00 | 62.97 |
| 5812 | CB | CYS D | 151 | 37.636 | 42.966 | 36.413 | 1.00 | 102.16 |
| 5813 | SG | CYS D | 151 | 38.287 | 44.417 | 35.527 | 1.00 | 102.16 |
| 5814 | N | THR D | 152 | 36.975 | 40.566 | 34.538 | 1.00 | 73.20 |
| 5815 | CA | THR D | 152 | 36.613 | 40.055 | 33.215 | 1.00 | 73.20 |
| 5816 | CB | THR D | 152 | 36.437 | 38.527 | 33.230 | 1.00 | 136.00 |
| 5817 | OG1 | THR D | 152 | 35.288 | 38.183 | 34.017 | 1.00 | 136.00 |
| 5818 | CG2 | THR D | 152 | 37.664 | 37.855 | 33.814 | 1.00 | 136.00 |
| 5819 | C | THR D | 152 | 35.286 | 40.688 | 32.830 | 1.00 | 73.20 |
| 5820 | O | THR D | 152 | 34.434 | 40.945 | 33.698 | 1.00 | 73.20 |
| 5821 | N | GLY D | 153 | 35.105 | 40.950 | 31.538 | 1.00 | 64.84 |
| 5822 | CA | GLY D | 153 | 33.863 | 41.572 | 31.099 | 1.00 | 64.84 |
| 5823 | C | GLY D | 153 | 33.682 | 41.463 | 29.609 | 1.00 | 64.84 |
| 5824 | O | GLY D | 153 | 34.636 | 41.184 | 28.899 | 1.00 | 64.84 |
| 5825 | N | LYS D | 154 | 32.462 | 41.680 | 29.133 | 1.00 | 72.61 |
| 5826 | CA | LYS D | 154 | 32.180 | 41.576 | 27.706 | 1.00 | 72.61 |
| 5827 | CB | LYS D | 154 | 30.881 | 40.800 | 27.484 | 1.00 | 205.73 |
| 5828 | CG | LYS D | 154 | 30.546 | 40.515 | 26.030 | 1.00 | 205.73 |
| 5829 | CD | LYS D | 154 | 29.274 | 39.697 | 25.956 | 1.00 | 205.73 |
| 5830 | CE | LYS D | 154 | 28.825 | 39.439 | 24.533 | 1.00 | 205.73 |
| 5831 | NZ | LYS D | 154 | 27.516 | 38.730 | 24.533 | 1.00 | 205.73 |
| 5832 | C | LYS D | 154 | 32.056 | 42.967 | 27.134 | 1.00 | 72.61 |
| 5833 | O | LYS D | 154 | 31.329 | 43.802 | 27.662 | 1.00 | 72.61 |
| 5834 | N | VAL D | 155 | 32.792 | 43.226 | 26.065 | 1.00 | 92.93 |
| 5835 | CA | VAL D | 155 | 32.751 | 44.532 | 25.426 | 1.00 | 92.93 |
| 5836 | CB | VAL D | 155 | 34.140 | 45.153 | 25.313 | 1.00 | 130.27 |
| 5837 | CG1 | VAL D | 155 | 34.065 | 46.494 | 24.609 | 1.00 | 130.27 |
| 5838 | CG2 | VAL D | 155 | 34.714 | 45.331 | 26.689 | 1.00 | 130.27 |
| 5839 | C | VAL D | 155 | 32.216 | 44.275 | 24.040 | 1.00 | 92.93 |
| 5840 | O | VAL D | 155 | 32.715 | 43.395 | 23.330 | 1.00 | 92.93 |
| 5841 | N | TRP D | 156 | 31.205 | 45.046 | 23.653 | 1.00 | 158.38 |
| 5842 | CA | TRP D | 156 | 30.579 | 44.860 | 22.358 | 1.00 | 158.38 |
| 5843 | CB | TRP D | 156 | 31.605 | 44.866 | 21.235 | 1.00 | 243.82 |
| 5844 | CG | TRP D | 156 | 32.236 | 46.155 | 21.100 | 1.00 | 243.82 |
| 5845 | CD2 | TRP D | 156 | 31.589 | 47.366 | 20.812 | 1.00 | 243.82 |
| 5846 | CE2 | TRP D | 156 | 32.575 | 48.376 | 20.781 | 1.00 | 243.82 |
| 5847 | CE3 | TRP D | 156 | 30.261 | 47.713 | 20.608 | 1.00 | 243.82 |
| 5848 | CD1 | TRP D | 156 | 33.550 | 46.428 | 21.198 | 1.00 | 243.82 |
| 5849 | NE1 | TRP D | 156 | 33.771 | 47.767 | 21.014 | 1.00 | 243.82 |
| 5850 | CZ2 | TRP D | 156 | 32.278 | 49.703 | 20.533 | 1.00 | 243.82 |
| 5851 | CZ3 | TRP D | 156 | 29.993 | 49.006 | 20.358 | 1.00 | 243.82 |
| 5852 | CH2 | TRP D | 156 | 30.983 | 49.997 | 20.320 | 1.00 | 243.82 |
| 5853 | C | TRP D | 156 | 29.982 | 43.492 | 22.407 | 1.00 | 158.38 |
| 5854 | O | TRP D | 156 | 28.886 | 43.299 | 22.908 | 1.00 | 158.38 |
| 5855 | N | GLN D | 157 | 30.752 | 42.528 | 21.924 | 1.00 | 148.04 |
| 5856 | CA | GLN D | 157 | 30.284 | 41.168 | 21.881 | 1.00 | 148.04 |
| 5857 | CB | GLN D | 157 | 29.612 | 40.948 | 20.533 | 1.00 | 249.45 |
| 5858 | CG | GLN D | 157 | 28.288 | 41.684 | 20.483 | 1.00 | 249.45 |
| 5859 | CD | GLN D | 157 | 27.435 | 41.306 | 21.676 | 1.00 | 249.45 |
| 5860 | OE1 | GLN D | 157 | 27.203 | 40.132 | 21.914 | 1.00 | 249.45 |
| 5861 | NE2 | GLN D | 157 | 26.963 | 42.292 | 22.421 | 1.00 | 249.45 |
| 5862 | C | GLN D | 157 | 31.348 | 40.127 | 22.150 | 1.00 | 148.04 |
| 5863 | O | GLN D | 157 | 31.140 | 38.936 | 21.912 | 1.00 | 148.04 |
| 5864 | N | LEU D | 158 | 32.488 | 40.579 | 22.665 | 1.00 | 85.54 |
| 5865 | CA | LEU D | 158 | 33.584 | 39.676 | 22.996 | 1.00 | 85.54 |
| 5866 | CB | LEU D | 158 | 34.779 | 39.917 | 22.073 | 1.00 | 127.62 |
| 5867 | CG | LEU D | 158 | 34.714 | 39.361 | 20.649 | 1.00 | 127.62 |
| 5868 | CD1 | LEU D | 158 | 36.094 | 38.823 | 20.329 | 1.00 | 127.62 |
| 5869 | CD2 | LEU D | 158 | 33.694 | 38.231 | 20.514 | 1.00 | 127.62 |
| 5870 | C | LEU D | 158 | 34.022 | 39.797 | 24.457 | 1.00 | 85.54 |
| 5871 | O | LEU D | 158 | 33.857 | 40.847 | 25.090 | 1.00 | 85.54 |
| 5872 | N | ASP D | 159 | 34.562 | 38.706 | 24.986 | 1.00 | 91.88 |
| 5873 | CA | ASP D | 159 | 35.024 | 38.662 | 26.363 | 1.00 | 91.88 |
| 5874 | CB | ASP D | 159 | 34.915 | 37.229 | 26.901 | 1.00 | 249.49 |
| 5875 | CG | ASP D | 159 | 33.518 | 36.644 | 26.743 | 1.00 | 249.49 |
| 5876 | OD1 | ASP D | 159 | 32.559 | 37.204 | 27.319 | 1.00 | 249.49 |
| 5877 | OD2 | ASP D | 159 | 33.380 | 35.620 | 26.039 | 1.00 | 249.49 |
| 5878 | C | ASP D | 159 | 36.476 | 39.142 | 26.462 | 1.00 | 91.88 |
| 5879 | O | ASP D | 159 | 37.270 | 38.921 | 25.543 | 1.00 | 91.88 |
| 5880 | N | TYR D | 160 | 36.818 | 39.807 | 27.568 | 1.00 | 90.02 |
| 5881 | CA | TYR D | 160 | 38.179 | 40.298 | 27.785 | 1.00 | 90.02 |
| 5882 | CB | TYR D | 160 | 38.334 | 41.742 | 27.323 | 1.00 | 132.54 |
| 5883 | CG | TYR D | 160 | 37.907 | 41.996 | 25.905 | 1.00 | 132.54 |
| 5884 | CD1 | TYR D | 160 | 36.605 | 42.362 | 25.617 | 1.00 | 132.54 |
| 5885 | CE1 | TYR D | 160 | 36.196 | 42.585 | 24.315 | 1.00 | 132.54 |
| 5886 | CD2 | TYR D | 160 | 38.800 | 41.857 | 24.851 | 1.00 | 132.54 |
| 5887 | CE2 | TYR D | 160 | 38.405 | 42.075 | 23.539 | 1.00 | 132.54 |
| 5888 | CZ | TYR D | 160 | 37.101 | 42.437 | 23.278 | 1.00 | 132.54 |
| 5889 | OH | TYR D | 160 | 36.697 | 42.638 | 21.977 | 1.00 | 132.54 |
| 5890 | C | TYR D | 160 | 38.594 | 40.222 | 29.239 | 1.00 | 90.02 |
| 5891 | O | TYR D | 160 | 37.782 | 40.394 | 30.143 | 1.00 | 90.02 |
| 5892 | N | GLU D | 161 | 39.884 | 39.979 | 29.436 | 1.00 | 92.36 |
| 5893 | CA | GLU D | 161 | 40.492 | 39.862 | 30.750 | 1.00 | 92.36 |
| 5894 | CB | GLU D | 161 | 41.247 | 38.536 | 30.815 | 1.00 | 148.75 |
| 5895 | CG | GLU D | 161 | 42.005 | 38.266 | 32.084 | 1.00 | 148.75 |
| 5896 | CD | GLU D | 161 | 42.398 | 36.810 | 32.189 | 1.00 | 148.75 |
| 5897 | OE1 | GLU D | 161 | 43.333 | 36.490 | 32.957 | 1.00 | 148.75 |
| 5898 | OE2 | GLU D | 161 | 41.757 | 35.985 | 31.506 | 1.00 | 148.75 |
| 5899 | C | GLU D | 161 | 41.448 | 41.051 | 30.923 | 1.00 | 92.36 |
| 5900 | O | GLU D | 161 | 42.157 | 41.424 | 29.985 | 1.00 | 92.36 |
| 5901 | N | SER D | 162 | 41.450 | 41.655 | 32.110 | 1.00 | 74.96 |
| 5902 | CA | SER D | 162 | 42.323 | 42.789 | 32.404 | 1.00 | 74.96 |
| 5903 | CB | SER D | 162 | 41.652 | 43.705 | 33.398 | 1.00 | 62.82 |
| 5904 | OG | SER D | 162 | 41.377 | 42.998 | 34.594 | 1.00 | 62.82 |
| 5905 | C | SER D | 162 | 43.671 | 42.370 | 32.989 | 1.00 | 74.96 |
| 5906 | O | SER D | 162 | 43.876 | 41.208 | 33.354 | 1.00 | 74.96 |
| 5907 | N | GLU D | 163 | 44.593 | 43.325 | 33.088 | 1.00 | 68.26 |
| 5908 | CA | GLU D | 163 | 45.929 | 43.034 | 33.625 | 1.00 | 68.26 |
| 5909 | CB | GLU D | 163 | 46.877 | 44.206 | 33.368 | 1.00 | 242.79 |
| 5910 | CG | GLU D | 163 | 47.352 | 44.338 | 31.925 | 1.00 | 242.79 |
| 5911 | CD | GLU D | 163 | 48.358 | 43.266 | 31.540 | 1.00 | 242.79 |
| 5912 | OE1 | GLU D | 163 | 49.400 | 43.158 | 32.222 | 1.00 | 242.79 |
| 5913 | OE2 | GLU D | 163 | 48.113 | 42.537 | 30.556 | 1.00 | 242.79 |
| 5914 | C | GLU D | 163 | 45.768 | 42.820 | 35.117 | 1.00 | 68.26 |
| 5915 | O | GLU D | 163 | 44.970 | 43.504 | 35.751 | 1.00 | 68.26 |
| 5916 | N | PRO D | 164 | 46.511 | 41.863 | 35.698 | 1.00 | 51.48 |
| 5917 | CD | PRO D | 164 | 47.539 | 40.983 | 35.148 | 1.00 | 112.85 |
| 5918 | CA | PRO D | 164 | 46.359 | 41.659 | 37.137 | 1.00 | 51.48 |
| 5919 | CB | PRO D | 164 | 47.112 | 40.364 | 37.367 | 1.00 | 112.85 |
| 5920 | CG | PRO D | 164 | 48.211 | 40.465 | 36.404 | 1.00 | 112.85 |
| 5921 | C | PRO D | 164 | 46.955 | 42.827 | 37.920 | 1.00 | 51.48 |
| 5922 | O | PRO D | 164 | 47.839 | 43.536 | 37.411 | 1.00 | 51.48 |
| 5923 | N | LEU D | 165 | 46.480 | 43.045 | 39.145 | 1.00 | 58.03 |
| 5924 | CA | LEU D | 165 | 46.988 | 44.134 | 39.944 | 1.00 | 58.03 |
| 5925 | CB | LEU D | 165 | 46.085 | 45.333 | 39.824 | 1.00 | 67.82 |
| 5926 | CG | LEU D | 165 | 46.417 | 46.436 | 40.816 | 1.00 | 67.82 |
| 5927 | CD1 | LEU D | 165 | 47.878 | 46.685 | 40.736 | 1.00 | 67.82 |
| 5928 | CD2 | LEU D | 165 | 45.653 | 47.711 | 40.495 | 1.00 | 67.82 |
| 5929 | C | LEU D | 165 | 47.080 | 43.744 | 41.384 | 1.00 | 58.03 |
| 5930 | O | LEU D | 165 | 46.082 | 43.313 | 41.957 | 1.00 | 58.03 |
| 5931 | N | ASN D | 166 | 48.274 | 43.892 | 41.964 | 1.00 | 50.98 |
| 5932 | CA | ASN D | 166 | 48.513 | 43.551 | 43.367 | 1.00 | 50.98 |
| 5933 | CB | ASN D | 166 | 49.984 | 43.249 | 43.618 | 1.00 | 110.65 |
| 5934 | CG | ASN D | 166 | 50.324 | 41.777 | 43.461 | 1.00 | 110.65 |
| 5935 | OD1 | ASN D | 166 | 49.514 | 40.900 | 43.700 | 1.00 | 110.65 |
| 5936 | ND2 | ASN D | 166 | 51.557 | 41.517 | 43.077 | 1.00 | 110.65 |
| 5937 | C | ASN D | 166 | 48.084 | 44.660 | 44.311 | 1.00 | 50.98 |
| 5938 | O | ASN D | 166 | 48.175 | 45.818 | 43.992 | 1.00 | 50.98 |
| 5939 | N | ILE D | 167 | 47.626 | 44.290 | 45.489 | 1.00 | 69.36 |
| 5940 | CA | ILE D | 167 | 47.167 | 45.267 | 46.443 | 1.00 | 69.36 |
| 5941 | CB | ILE D | 167 | 45.659 | 45.375 | 46.397 | 1.00 | 42.00 |
| 5942 | CG2 | ILE D | 167 | 45.152 | 46.062 | 47.661 | 1.00 | 42.00 |
| 5943 | CG1 | ILE D | 167 | 45.241 | 46.105 | 45.129 | 1.00 | 42.00 |
| 5944 | CD1 | ILE D | 167 | 43.748 | 46.402 | 45.115 | 1.00 | 42.00 |
| 5945 | C | ILE D | 167 | 47.557 | 44.842 | 47.833 | 1.00 | 69.36 |
| 5946 | O | ILE D | 167 | 47.366 | 43.682 | 48.218 | 1.00 | 69.36 |
| 5947 | N | THR D | 168 | 48.090 | 45.774 | 48.603 | 1.00 | 69.22 |
| 5948 | CA | THR D | 168 | 48.480 | 45.418 | 49.945 | 1.00 | 69.22 |
| 5949 | CB | THR D | 168 | 49.988 | 45.453 | 50.107 | 1.00 | 70.81 |
| 5950 | OG1 | THR D | 168 | 50.575 | 44.544 | 49.169 | 1.00 | 70.81 |
| 5951 | CG2 | THR D | 168 | 50.372 | 45.030 | 51.511 | 1.00 | 70.81 |
| 5952 | C | THR D | 168 | 47.847 | 46.301 | 50.987 | 1.00 | 69.22 |
| 5953 | O | THR D | 168 | 47.754 | 47.509 | 50.828 | 1.00 | 69.22 |
| 5954 | N | VAL D | 169 | 47.387 | 45.670 | 52.051 | 1.00 | 66.57 |
| 5955 | CA | VAL D | 169 | 46.774 | 46.361 | 53.155 | 1.00 | 66.57 |
| 5956 | CB | VAL D | 169 | 45.379 | 45.806 | 53.417 | 1.00 | 62.74 |
| 5957 | CG1 | VAL D | 169 | 44.945 | 46.137 | 54.819 | 1.00 | 62.74 |
| 5958 | CG2 | VAL D | 169 | 44.418 | 46.381 | 52.416 | 1.00 | 62.74 |
| 5959 | C | VAL D | 169 | 47.693 | 46.061 | 54.334 | 1.00 | 66.57 |
| 5960 | O | VAL D | 169 | 47.740 | 44.932 | 54.805 | 1.00 | 66.57 |
| 5961 | N | ILE D | 170 | 48.460 | 47.051 | 54.780 | 1.00 | 82.22 |
| 5962 | CA | ILE D | 170 | 49.360 | 46.864 | 55.913 | 1.00 | 82.22 |
| 5963 | CB | ILE D | 170 | 50.599 | 47.738 | 55.759 | 1.00 | 114.31 |
| 5964 | CG2 | ILE D | 170 | 51.201 | 47.504 | 54.406 | 1.00 | 114.31 |
| 5965 | CG1 | ILE D | 170 | 50.232 | 49.218 | 55.860 | 1.00 | 114.31 |
| 5966 | CD1 | ILE D | 170 | 51.416 | 50.187 | 55.670 | 1.00 | 114.31 |
| 5967 | C | ILE D | 170 | 48.613 | 47.237 | 57.189 | 1.00 | 82.22 |
| 5968 | O | ILE D | 170 | 47.459 | 47.676 | 57.122 | 1.00 | 82.22 |
| 5969 | N | LYS D | 171 | 49.245 | 47.053 | 58.347 | 1.00 | 108.79 |
| 5970 | CA | LYS D | 171 | 48.598 | 47.390 | 59.620 | 1.00 | 108.79 |
| 5971 | CB | LYS D | 171 | 48.214 | 46.117 | 60.360 | 1.00 | 188.56 |
| 5972 | CG | LYS D | 171 | 49.380 | 45.194 | 60.614 | 1.00 | 188.56 |
| 5973 | CD | LYS D | 171 | 48.910 | 43.762 | 60.800 | 1.00 | 188.56 |
| 5974 | CE | LYS D | 171 | 47.946 | 43.624 | 61.976 | 1.00 | 188.56 |
| 5975 | NZ | LYS D | 171 | 47.459 | 42.220 | 62.130 | 1.00 | 188.56 |
| 5976 | C | LYS D | 171 | 49.453 | 48.270 | 60.524 | 1.00 | 108.79 |
| 5977 | O | LYS D | 171 | 48.981 | 48.761 | 61.549 | 1.00 | 108.79 |
| 5978 | C1 | NAG D | 221 | 40.344 | 65.629 | 28.022 | 1.00 | 249.77 |
| 5979 | C2 | NAG D | 221 | 39.010 | 64.922 | 27.810 | 1.00 | 249.77 |
| 5980 | N2 | NAG D | 221 | 39.203 | 63.489 | 27.903 | 1.00 | 249.77 |
| 5981 | C7 | NAG D | 221 | 38.191 | 62.705 | 28.261 | 1.00 | 249.77 |
| 5982 | O7 | NAG D | 221 | 37.073 | 63.139 | 28.545 | 1.00 | 249.77 |
| 5983 | C8 | NAG D | 221 | 38.462 | 61.211 | 28.324 | 1.00 | 249.77 |
| 5984 | C3 | NAG D | 221 | 38.434 | 65.256 | 26.441 | 1.00 | 249.77 |
| 5985 | O3 | NAG D | 221 | 37.116 | 64.735 | 26.342 | 1.00 | 249.77 |
| 5986 | C4 | NAG D | 221 | 38.404 | 66.763 | 26.173 | 1.00 | 249.77 |
| 5987 | O4 | NAG D | 221 | 38.077 | 66.947 | 24.777 | 1.00 | 249.77 |
| 5988 | C5 | NAG D | 221 | 39.780 | 67.394 | 26.498 | 1.00 | 249.77 |
| 5989 | O5 | NAG D | 221 | 40.191 | 67.042 | 27.838 | 1.00 | 249.77 |
| 5990 | C6 | NAG D | 221 | 39.770 | 68.913 | 26.439 | 1.00 | 249.77 |
| 5991 | O6 | NAG D | 221 | 38.854 | 69.459 | 27.379 | 1.00 | 249.77 |
| 5992 | C1 | NAG D | 222 | 37.635 | 68.189 | 24.343 | 1.00 | 233.91 |
| 5993 | C2 | NAG D | 222 | 36.436 | 68.019 | 23.396 | 1.00 | 233.91 |
| 5994 | N2 | NAG D | 222 | 35.346 | 67.346 | 24.082 | 1.00 | 233.91 |
| 5995 | C7 | NAG D | 222 | 34.173 | 67.955 | 24.234 | 1.00 | 233.91 |
| 5996 | O7 | NAG D | 222 | 33.947 | 69.093 | 23.816 | 1.00 | 233.91 |
| 5997 | C8 | NAG D | 222 | 33.082 | 67.183 | 24.963 | 1.00 | 233.91 |
| 5998 | C3 | NAG D | 222 | 36.855 | 67.215 | 22.156 | 1.00 | 233.91 |
| 5999 | O3 | NAG D | 222 | 35.790 | 67.196 | 21.217 | 1.00 | 233.91 |
| 6000 | C4 | NAG D | 222 | 38.102 | 67.829 | 21.503 | 1.00 | 233.91 |
| 6001 | O4 | NAG D | 222 | 38.567 | 66.974 | 20.471 | 1.00 | 233.91 |
| 6002 | C5 | NAG D | 222 | 39.211 | 68.020 | 22.542 | 1.00 | 233.91 |
| 6003 | O5 | NAG D | 222 | 38.722 | 68.817 | 23.644 | 1.00 | 233.91 |
| 6004 | C6 | NAG D | 222 | 40.435 | 68.722 | 21.989 | 1.00 | 233.91 |
| 6005 | O6 | NAG D | 222 | 41.628 | 68.076 | 22.406 | 1.00 | 233.91 |
| 6006 | C1 | NAG D | 242 | 59.627 | 58.578 | 32.960 | 1.00 | 107.57 |
| 6007 | C2 | NAG D | 242 | 59.450 | 58.871 | 31.486 | 1.00 | 107.57 |
| 6008 | N2 | NAG D | 242 | 59.010 | 60.232 | 31.316 | 1.00 | 107.57 |
| 6009 | C7 | NAG D | 242 | 59.707 | 61.044 | 30.534 | 1.00 | 107.57 |
| 6010 | O7 | NAG D | 242 | 60.732 | 60.679 | 29.950 | 1.00 | 107.57 |
| 6011 | C8 | NAG D | 242 | 59.199 | 62.478 | 30.373 | 1.00 | 107.57 |
| 6012 | C3 | NAG D | 242 | 58.412 | 57.932 | 30.887 | 1.00 | 107.57 |
| 6013 | O3 | NAG D | 242 | 58.316 | 58.138 | 29.483 | 1.00 | 107.57 |
| 6014 | C4 | NAG D | 242 | 58.806 | 56.496 | 31.148 | 1.00 | 107.57 |

| | | | | | | | | | |
|---|---|---|---|---|---|---|---|---|---|
| 6015 | O4 | NAG | D | 242 | 57.728 | 55.629 | 30.752 | 1.00 | 107.57 |
| 6016 | C5 | NAG | D | 242 | 59.118 | 56.268 | 32.625 | 1.00 | 107.57 |
| 6017 | O5 | NAG | D | 242 | 60.064 | 57.236 | 33.114 | 1.00 | 107.57 |
| 6018 | C6 | NAG | D | 242 | 59.783 | 54.930 | 32.783 | 1.00 | 107.57 |
| 6019 | O6 | NAG | D | 242 | 59.082 | 54.107 | 33.697 | 1.00 | 107.57 |
| 6020 | C1 | NAG | D | 243 | 57.985 | 54.762 | 29.705 | 1.00 | 125.30 |
| 6021 | C2 | NAG | D | 243 | 57.074 | 53.527 | 29.789 | 1.00 | 125.30 |
| 6022 | N2 | NAG | D | 243 | 57.321 | 52.782 | 31.013 | 1.00 | 125.30 |
| 6023 | C7 | NAG | D | 243 | 56.303 | 52.357 | 31.758 | 1.00 | 125.30 |
| 6024 | O7 | NAG | D | 243 | 55.129 | 52.583 | 31.473 | 1.00 | 125.30 |
| 6025 | C8 | NAG | D | 243 | 56.634 | 51.574 | 33.018 | 1.00 | 125.30 |
| 6026 | C3 | NAG | D | 243 | 57.345 | 52.629 | 28.586 | 1.00 | 125.30 |
| 6027 | O3 | NAG | D | 243 | 56.458 | 51.521 | 28.595 | 1.00 | 125.30 |
| 6028 | C4 | NAG | D | 243 | 57.191 | 53.414 | 27.277 | 1.00 | 125.30 |
| 6029 | O4 | NAG | D | 243 | 57.612 | 52.582 | 26.156 | 1.00 | 125.30 |
| 6030 | C5 | NAG | D | 243 | 58.083 | 54.659 | 27.339 | 1.00 | 125.30 |
| 6031 | O5 | NAG | D | 243 | 57.744 | 55.459 | 28.483 | 1.00 | 125.30 |
| 6032 | C6 | NAG | D | 243 | 57.985 | 55.549 | 26.119 | 1.00 | 125.30 |
| 6033 | O6 | NAG | D | 243 | 56.713 | 56.172 | 26.043 | 1.00 | 125.30 |
| 6034 | C1 | MAN | D | 244 | 56.846 | 52.424 | 25.031 | 1.00 | 205.85 |
| 6035 | C2 | MAN | D | 244 | 55.417 | 51.844 | 25.171 | 1.00 | 205.85 |
| 6036 | O2 | MAN | D | 244 | 54.487 | 52.897 | 25.184 | 1.00 | 205.85 |
| 6037 | C3 | MAN | D | 244 | 55.275 | 51.012 | 23.858 | 1.00 | 205.85 |
| 6038 | O3 | MAN | D | 244 | 54.016 | 50.380 | 23.757 | 1.00 | 205.85 |
| 6039 | C4 | MAN | D | 244 | 55.586 | 51.831 | 22.569 | 1.00 | 205.85 |
| 6040 | O4 | MAN | D | 244 | 55.419 | 51.015 | 21.411 | 1.00 | 205.85 |
| 6041 | C5 | MAN | D | 244 | 57.054 | 52.305 | 22.669 | 1.00 | 205.85 |
| 6042 | O5 | MAN | D | 244 | 57.244 | 53.154 | 23.833 | 1.00 | 205.85 |
| 6043 | C6 | MAN | D | 244 | 57.597 | 52.991 | 21.412 | 1.00 | 205.85 |
| 6044 | O6 | MAN | D | 244 | 57.221 | 54.349 | 21.357 | 1.00 | 205.85 |
| 6045 | C1 | NAG | D | 250 | 45.992 | 76.510 | 37.679 | 1.00 | 248.68 |
| 6046 | C2 | NAG | D | 250 | 44.579 | 76.931 | 38.128 | 1.00 | 248.68 |
| 6047 | N2 | NAG | D | 250 | 44.536 | 77.116 | 39.567 | 1.00 | 248.68 |
| 6048 | C7 | NAG | D | 250 | 44.384 | 78.333 | 40.083 | 1.00 | 248.68 |
| 6049 | O7 | NAG | D | 250 | 44.277 | 79.347 | 39.391 | 1.00 | 248.68 |
| 6050 | C8 | NAG | D | 250 | 44.348 | 78.442 | 41.599 | 1.00 | 248.68 |
| 6051 | C3 | NAG | D | 250 | 43.573 | 75.849 | 37.715 | 1.00 | 248.68 |
| 6052 | O3 | NAG | D | 250 | 42.252 | 76.265 | 38.034 | 1.00 | 248.68 |
| 6053 | C4 | NAG | D | 250 | 43.682 | 75.570 | 36.213 | 1.00 | 248.68 |
| 6054 | O4 | NAG | D | 250 | 42.841 | 74.477 | 35.869 | 1.00 | 248.68 |
| 6055 | C5 | NAG | D | 250 | 45.139 | 75.244 | 35.834 | 1.00 | 248.68 |
| 6056 | O5 | NAG | D | 250 | 46.017 | 76.312 | 36.259 | 1.00 | 248.68 |
| 6057 | C6 | NAG | D | 250 | 45.335 | 75.070 | 34.335 | 1.00 | 248.68 |
| 6058 | O6 | NAG | D | 250 | 46.713 | 75.089 | 33.986 | 1.00 | 248.68 |
| 6059 | C1 | NAG | D | 274 | 63.247 | 69.025 | 55.540 | 1.00 | 209.92 |
| 6060 | C2 | NAG | D | 274 | 62.953 | 68.056 | 56.695 | 1.00 | 209.92 |
| 6061 | N2 | NAG | D | 274 | 61.768 | 68.477 | 57.416 | 1.00 | 209.92 |
| 6062 | C7 | NAG | D | 274 | 61.053 | 67.585 | 58.098 | 1.00 | 209.92 |
| 6063 | O7 | NAG | D | 274 | 61.342 | 66.390 | 58.145 | 1.00 | 209.92 |
| 6064 | C8 | NAG | D | 274 | 59.826 | 68.096 | 58.835 | 1.00 | 209.92 |
| 6065 | C3 | NAG | D | 274 | 64.147 | 68.007 | 57.654 | 1.00 | 209.92 |
| 6066 | O3 | NAG | D | 274 | 63.927 | 67.009 | 58.639 | 1.00 | 209.92 |
| 6067 | C4 | NAG | D | 274 | 65.443 | 67.703 | 56.893 | 1.00 | 209.92 |
| 6068 | O4 | NAG | D | 274 | 66.552 | 67.817 | 57.775 | 1.00 | 209.92 |
| 6069 | C5 | NAG | D | 274 | 65.610 | 68.683 | 55.725 | 1.00 | 209.92 |
| 6070 | O5 | NAG | D | 274 | 64.452 | 68.631 | 54.865 | 1.00 | 209.92 |
| 6071 | C6 | NAG | D | 274 | 66.820 | 68.373 | 54.862 | 1.00 | 209.92 |
| 6072 | O6 | NAG | D | 274 | 66.810 | 69.142 | 53.667 | 1.00 | 209.92 |
| 6073 | C1 | NAG | D | 335 | 32.860 | 53.594 | 38.525 | 1.00 | 187.23 |
| 6074 | C2 | NAG | D | 335 | 32.657 | 54.924 | 39.281 | 1.00 | 187.23 |
| 6075 | N2 | NAG | D | 335 | 32.302 | 54.604 | 40.651 | 1.00 | 187.23 |
| 6076 | C7 | NAG | D | 335 | 33.089 | 54.970 | 41.656 | 1.00 | 187.23 |
| 6077 | O7 | NAG | D | 335 | 34.133 | 55.601 | 41.498 | 1.00 | 187.23 |
| 6078 | C8 | NAG | D | 335 | 32.640 | 54.583 | 43.054 | 1.00 | 187.23 |
| 6079 | C3 | NAG | D | 335 | 31.561 | 55.826 | 38.691 | 1.00 | 187.23 |
| 6080 | O3 | NAG | D | 335 | 31.736 | 57.155 | 39.169 | 1.00 | 187.23 |
| 6081 | C4 | NAG | D | 335 | 31.606 | 55.833 | 37.168 | 1.00 | 187.23 |
| 6082 | O4 | NAG | D | 335 | 30.534 | 56.616 | 36.658 | 1.00 | 187.23 |
| 6083 | C5 | NAG | D | 335 | 31.498 | 54.394 | 36.668 | 1.00 | 187.23 |
| 6084 | O5 | NAG | D | 335 | 32.666 | 53.657 | 37.089 | 1.00 | 187.23 |
| 6085 | C6 | NAG | D | 335 | 31.442 | 54.317 | 35.144 | 1.00 | 187.23 |
| 6086 | O6 | NAG | D | 335 | 30.243 | 53.705 | 34.692 | 1.00 | 187.23 |
| 6087 | C1 | NAG | D | 340 | 36.447 | 48.280 | 60.935 | 1.00 | 247.88 |
| 6088 | C2 | NAG | D | 340 | 37.563 | 48.157 | 61.941 | 1.00 | 247.88 |
| 6089 | N2 | NAG | D | 340 | 38.786 | 47.736 | 61.296 | 1.00 | 247.88 |
| 6090 | C7 | NAG | D | 340 | 39.907 | 48.420 | 61.502 | 1.00 | 247.88 |
| 6091 | O7 | NAG | D | 340 | 39.959 | 49.402 | 62.248 | 1.00 | 247.88 |
| 6092 | C8 | NAG | D | 340 | 41.160 | 47.954 | 60.781 | 1.00 | 247.88 |
| 6093 | C3 | NAG | D | 340 | 37.180 | 47.173 | 63.025 | 1.00 | 247.88 |
| 6094 | O3 | NAG | D | 340 | 38.213 | 47.101 | 64.002 | 1.00 | 247.88 |
| 6095 | C4 | NAG | D | 340 | 35.881 | 47.637 | 63.677 | 1.00 | 247.88 |
| 6096 | O4 | NAG | D | 340 | 35.406 | 46.605 | 64.547 | 1.00 | 247.88 |
| 6097 | C5 | NAG | D | 340 | 34.778 | 47.988 | 62.613 | 1.00 | 247.88 |
| 6098 | O5 | NAG | D | 340 | 35.305 | 48.810 | 61.587 | 1.00 | 247.88 |
| 6099 | C6 | NAG | D | 340 | 33.729 | 48.850 | 63.239 | 1.00 | 247.88 |
| 6100 | O6 | NAG | D | 340 | 33.003 | 49.565 | 62.297 | 1.00 | 247.88 |
| 6101 | C1 | NAG | D | 366 | 51.975 | 40.156 | 42.859 | 1.00 | 179.92 |
| 6102 | C2 | NAG | D | 366 | 53.015 | 40.152 | 41.753 | 1.00 | 179.92 |
| 6103 | N2 | NAG | D | 366 | 52.433 | 40.714 | 40.551 | 1.00 | 179.92 |
| 6104 | C7 | NAG | D | 366 | 52.553 | 42.013 | 40.303 | 1.00 | 179.92 |
| 6105 | O7 | NAG | D | 366 | 53.160 | 42.786 | 41.048 | 1.00 | 179.92 |
| 6106 | C8 | NAG | D | 366 | 51.908 | 42.532 | 39.029 | 1.00 | 179.92 |
| 6107 | C3 | NAG | D | 366 | 53.483 | 38.733 | 41.488 | 1.00 | 179.92 |
| 6108 | O3 | NAG | D | 366 | 54.558 | 38.758 | 40.562 | 1.00 | 179.92 |
| 6109 | C4 | NAG | D | 366 | 53.939 | 38.053 | 42.783 | 1.00 | 179.92 |
| 6110 | O4 | NAG | D | 366 | 54.150 | 36.651 | 42.516 | 1.00 | 179.92 |
| 6111 | C5 | NAG | D | 366 | 52.883 | 38.216 | 43.899 | 1.00 | 179.92 |
| 6112 | O5 | NAG | D | 366 | 52.522 | 39.602 | 44.056 | 1.00 | 179.92 |
| 6113 | C6 | NAG | D | 366 | 53.364 | 37.740 | 45.257 | 1.00 | 179.92 |
| 6114 | O6 | NAG | D | 366 | 52.346 | 37.880 | 46.242 | 1.00 | 179.92 |
| 6115 | C1 | NAG | D | 367 | 55.386 | 36.120 | 42.861 | 1.00 | 249.52 |
| 6116 | C2 | NAG | D | 367 | 55.270 | 34.606 | 43.041 | 1.00 | 249.52 |
| 6117 | N2 | NAG | D | 367 | 54.288 | 34.289 | 44.061 | 1.00 | 249.52 |
| 6118 | C7 | NAG | D | 367 | 53.121 | 33.757 | 43.710 | 1.00 | 249.52 |
| 6119 | O7 | NAG | D | 367 | 52.823 | 33.510 | 42.539 | 1.00 | 249.52 |
| 6120 | C8 | NAG | D | 367 | 52.132 | 33.450 | 44.825 | 1.00 | 249.52 |
| 6121 | C3 | NAG | D | 367 | 56.643 | 34.041 | 43.413 | 1.00 | 249.52 |
| 6122 | O3 | NAG | D | 367 | 56.568 | 32.629 | 43.535 | 1.00 | 249.52 |
| 6123 | C4 | NAG | D | 367 | 57.655 | 34.411 | 42.327 | 1.00 | 249.52 |
| 6124 | O4 | NAG | D | 367 | 58.951 | 33.973 | 42.709 | 1.00 | 249.52 |
| 6125 | C5 | NAG | D | 367 | 57.659 | 35.931 | 42.108 | 1.00 | 249.52 |
| 6126 | O5 | NAG | D | 367 | 56.321 | 36.405 | 41.807 | 1.00 | 249.52 |
| 6127 | C6 | NAG | D | 367 | 58.553 | 36.343 | 40.953 | 1.00 | 249.52 |
| 6128 | O6 | NAG | D | 367 | 57.795 | 36.836 | 39.858 | 1.00 | 249.52 |
| 6129 | CB | LYS | E | 4 | 12.130 | 63.790 | 1.727 | 1.00 | 181.25 |
| 6130 | CG | LYS | E | 4 | 10.709 | 63.348 | 1.434 | 1.00 | 181.25 |
| 6131 | CD | LYS | E | 4 | 9.964 | 63.056 | 2.721 | 1.00 | 181.25 |
| 6132 | CE | LYS | E | 4 | 8.534 | 62.631 | 2.447 | 1.00 | 181.25 |
| 6133 | NZ | LYS | E | 4 | 7.791 | 62.349 | 3.709 | 1.00 | 181.25 |
| 6134 | C | LYS | E | 4 | 12.157 | 65.259 | −0.281 | 1.00 | 249.30 |
| 6135 | O | LYS | E | 4 | 11.355 | 65.991 | 0.294 | 1.00 | 249.30 |
| 6136 | N | LYS | E | 4 | 14.286 | 64.661 | 0.874 | 1.00 | 249.30 |
| 6137 | CA | LYS | E | 4 | 12.924 | 64.186 | 0.485 | 1.00 | 249.30 |
| 6138 | N | PRO | E | 5 | 12.400 | 65.365 | −1.597 | 1.00 | 120.68 |
| 6139 | CD | PRO | E | 5 | 13.529 | 64.775 | −2.329 | 1.00 | 144.78 |
| 6140 | CA | PRO | E | 5 | 11.713 | 66.365 | −2.422 | 1.00 | 120.68 |
| 6141 | CB | PRO | E | 5 | 12.699 | 66.600 | −3.566 | 1.00 | 144.78 |
| 6142 | CG | PRO | E | 5 | 13.298 | 65.263 | −3.751 | 1.00 | 144.78 |
| 6143 | C | PRO | E | 5 | 10.345 | 65.902 | −2.912 | 1.00 | 120.68 |
| 6144 | O | PRO | E | 5 | 10.065 | 64.705 | −2.963 | 1.00 | 120.68 |
| 6145 | N | LYS | E | 6 | 9.489 | 66.856 | −3.263 | 1.00 | 141.31 |
| 6146 | CA | LYS | E | 6 | 8.153 | 66.534 | −3.743 | 1.00 | 141.31 |
| 6147 | CB | LYS | E | 6 | 7.152 | 66.622 | −2.590 | 1.00 | 196.63 |
| 6148 | CG | LYS | E | 6 | 5.747 | 66.182 | −2.959 | 1.00 | 196.63 |
| 6149 | CD | LYS | E | 6 | 4.834 | 66.110 | −1.741 | 1.00 | 196.63 |
| 6150 | CE | LYS | E | 6 | 3.443 | 65.623 | −2.137 | 1.00 | 196.63 |
| 6151 | NZ | LYS | E | 6 | 2.521 | 65.453 | −0.973 | 1.00 | 196.63 |
| 6152 | C | LYS | E | 6 | 7.735 | 67.460 | −4.883 | 1.00 | 141.31 |
| 6153 | O | LYS | E | 6 | 7.596 | 68.669 | −4.698 | 1.00 | 141.31 |
| 6154 | N | VAL | E | 7 | 7.526 | 66.874 | −6.059 | 1.00 | 81.07 |
| 6155 | CA | VAL | E | 7 | 7.145 | 67.622 | −7.259 | 1.00 | 81.07 |
| 6156 | CB | VAL | E | 7 | 7.188 | 66.745 | −8.530 | 1.00 | 76.53 |
| 6157 | CG1 | VAL | E | 7 | 6.965 | 67.610 | −9.757 | 1.00 | 76.53 |
| 6158 | CG2 | VAL | E | 7 | 8.488 | 66.003 | −8.626 | 1.00 | 76.53 |
| 6159 | C | VAL | E | 7 | 5.738 | 68.181 | −7.212 | 1.00 | 81.07 |
| 6160 | O | VAL | E | 7 | 4.778 | 67.426 | −7.151 | 1.00 | 81.07 |
| 6161 | N | SER | E | 8 | 5.606 | 69.498 | −7.268 | 1.00 | 146.33 |
| 6162 | CA | SER | E | 8 | 4.287 | 70.111 | −7.266 | 1.00 | 146.33 |
| 6163 | CB | SER | E | 8 | 4.268 | 71.325 | −6.332 | 1.00 | 208.51 |
| 8164 | OG | SER | E | 8 | 5.288 | 72.253 | −6.669 | 1.00 | 208.51 |
| 6165 | C | SER | E | 8 | 3.948 | 70.536 | −8.692 | 1.00 | 146.33 |
| 6166 | O | SER | E | 8 | 4.829 | 70.605 | −9.548 | 1.00 | 146.33 |
| 6167 | N | LEU | E | 9 | 2.671 | 70.806 | −8.946 | 1.00 | 130.86 |
| 6168 | CA | LEU | E | 9 | 2.221 | 71.236 | −10.269 | 1.00 | 130.86 |
| 6169 | CB | LEU | E | 9 | 1.358 | 70.166 | −10.929 | 1.00 | 129.83 |
| 6170 | CG | LEU | E | 9 | 1.921 | 68.783 | −11.217 | 1.00 | 129.83 |
| 6171 | CD1 | LEU | E | 9 | 1.089 | 68.115 | −12.291 | 1.00 | 129.83 |
| 6172 | CD2 | LEU | E | 9 | 3.337 | 68.911 | −11.693 | 1.00 | 129.83 |
| 6173 | C | LEU | E | 9 | 1.393 | 72.513 | −10.206 | 1.00 | 130.86 |
| 6174 | O | LEU | E | 9 | 0.783 | 72.822 | −9.184 | 1.00 | 130.86 |
| 6175 | N | ASN | E | 10 | 1.356 | 73.248 | −11.311 | 1.00 | 238.98 |
| 6176 | CA | ASN | E | 10 | 0.572 | 74.473 | −11.372 | 1.00 | 238.98 |
| 6177 | CB | ASN | E | 10 | 1.327 | 75.629 | −10.726 | 1.00 | 166.05 |
| 6178 | CG | ASN | E | 10 | 0.451 | 76.844 | −10.530 | 1.00 | 166.05 |
| 6179 | OD1 | ASN | E | 10 | −0.489 | 76.822 | −9.737 | 1.00 | 166.05 |
| 6180 | ND2 | ASN | E | 10 | 0.745 | 77.911 | −11.263 | 1.00 | 166.05 |
| 6181 | C | ASN | E | 10 | 0.235 | 74.821 | −12.817 | 1.00 | 238.98 |
| 6182 | O | ASN | E | 10 | 1.112 | 75.191 | −13.598 | 1.00 | 238.98 |
| 6183 | N | PRO | E | 11 | −1.053 | 74.715 | −13.195 | 1.00 | 125.56 |
| 6184 | CD | PRO | E | 11 | −1.439 | 74.939 | −14.595 | 1.00 | 163.67 |
| 6185 | CA | PRO | E | 11 | −2.214 | 74.304 | −12.392 | 1.00 | 125.56 |
| 6186 | CB | PRO | E | 11 | −3.341 | 74.258 | −13.429 | 1.00 | 163.67 |
| 6187 | CG | PRO | E | 11 | −2.903 | 75.247 | −14.467 | 1.00 | 163.67 |
| 6188 | C | PRO | E | 11 | −2.053 | 72.952 | −11.676 | 1.00 | 125.56 |
| 6189 | O | PRO | E | 11 | −1.179 | 72.163 | −12.027 | 1.00 | 125.56 |
| 6190 | N | PRO | E | 12 | −2.887 | 72.668 | −10.663 | 1.00 | 68.14 |
| 6191 | CD | PRO | E | 12 | −3.978 | 73.517 | −10.138 | 1.00 | 156.84 |
| 6192 | CA | PRO | E | 12 | −2.826 | 71.412 | −9.907 | 1.00 | 68.14 |
| 6193 | CB | PRO | E | 12 | −3.863 | 71.600 | −8.802 | 1.00 | 156.84 |
| 6194 | CG | PRO | E | 12 | −4.037 | 73.077 | −8.709 | 1.00 | 156.84 |
| 6195 | C | PRO | E | 12 | −3.214 | 70.234 | −10.814 | 1.00 | 68.14 |
| 6196 | O | PRO | E | 12 | −2.835 | 69.084 | −10.562 | 1.00 | 68.14 |
| 6197 | N | TRP | E | 13 | −3.987 | 70.547 | −11.857 | 1.00 | 90.03 |
| 6198 | CA | TRP | E | 13 | −4.488 | 69.551 | −12.800 | 1.00 | 90.03 |
| 6199 | CB | TRP | E | 13 | −5.267 | 70.231 | −13.916 | 1.00 | 120.89 |
| 6200 | CG | TRP | E | 13 | −6.235 | 71.215 | −13.407 | 1.00 | 120.89 |
| 6201 | CD2 | TRP | E | 13 | −7.024 | 71.111 | −12.227 | 1.00 | 120.89 |
| 6202 | CE2 | TRP | E | 13 | −7.750 | 72.310 | −12.102 | 1.00 | 120.89 |
| 6203 | CE3 | TRP | E | 13 | −7.191 | 70.121 | −11.255 | 1.00 | 120.89 |
| 6204 | CD1 | TRP | E | 13 | −6.509 | 72.430 | −13.945 | 1.00 | 120.89 |
| 6205 | NE1 | TRP | E | 13 | −7.418 | 73.100 | −13.167 | 1.00 | 120.89 |
| 6206 | CZ2 | TRP | E | 13 | −8.627 | 72.548 | −11.042 | 1.00 | 120.89 |
| 6207 | CZ3 | TRP | E | 13 | −8.065 | 70.359 | −10.204 | 1.00 | 120.89 |
| 6208 | CH2 | TRP | E | 13 | −8.768 | 71.561 | −10.104 | 1.00 | 120.89 |
| 6209 | C | TRP | E | 13 | −3.377 | 68.727 | −13.398 | 1.00 | 90.03 |
| 6210 | O | TRP | E | 13 | −2.479 | 69.264 | −14.031 | 1.00 | 90.03 |
| 6211 | N | ASN | E | 14 | −3.443 | 67.415 | −13.192 | 1.00 | 80.76 |
| 6212 | CA | ASN | E | 14 | −2.431 | 66.516 | −13.720 | 1.00 | 80.76 |
| 6213 | CB | ASN | E | 14 | −1.883 | 65.579 | −12.622 | 1.00 | 101.28 |
| 6214 | CG | ASN | E | 14 | −2.896 | 64.571 | −12.133 | 1.00 | 101.28 |
| 6215 | OD1 | ASN | E | 14 | −3.979 | 64.933 | −11.674 | 1.00 | 101.28 |
| 6216 | ND2 | ASN | E | 14 | −2.542 | 63.293 | −12.211 | 1.00 | 101.28 |
| 6217 | C | ASN | E | 14 | −2.917 | 65.715 | −14.921 | 1.00 | 80.76 |
| 6218 | O | ASN | E | 14 | −2.303 | 64.709 | −15.288 | 1.00 | 80.76 |
| 6219 | N | ARG | E | 15 | −4.026 | 66.154 | −15.523 | 1.00 | 74.26 |
| 6220 | CA | ARG | E | 15 | −4.554 | 65.520 | −16.732 | 1.00 | 74.26 |
| 6221 | CB | ARG | E | 15 | −5.855 | 64.779 | −16.490 | 1.00 | 82.67 |
| 6222 | CG | ARG | E | 15 | −5.888 | 63.996 | −15.236 | 1.00 | 82.67 |
| 6223 | CD | ARG | E | 15 | −7.202 | 63.278 | −15.142 | 1.00 | 82.67 |
| 6224 | NE | ARG | E | 15 | −7.314 | 62.199 | −16.114 | 1.00 | 82.67 |
| 6225 | CZ | ARG | E | 15 | −8.470 | 61.801 | −16.627 | 1.00 | 82.67 |
| 6226 | NH1 | ARG | E | 15 | −9.582 | 62.415 | −16.260 | 1.00 | 82.67 |
| 6227 | NH2 | ARG | E | 15 | −8.523 | 60.784 | −17.483 | 1.00 | 82.67 |
| 6228 | C | ARG | E | 15 | −4.860 | 66.730 | −17.570 | 1.00 | 74.26 |
| 6229 | O | ARG | E | 15 | −5.753 | 67.509 | −17.232 | 1.00 | 74.26 |
| 6230 | N | ILE | E | 16 | −4.116 | 66.920 | −18.647 | 1.00 | 117.86 |
| 6231 | CA | ILE | E | 16 | −4.363 | 68.085 | −19.460 | 1.00 | 117.86 |
| 6232 | CB | ILE | E | 16 | −3.213 | 69.070 | −19.378 | 1.00 | 89.68 |
| 6233 | CG2 | ILE | E | 16 | −3.128 | 69.663 | −17.980 | 1.00 | 89.68 |
| 6234 | CG1 | ILE | E | 16 | −1.917 | 68.359 | −19.758 | 1.00 | 89.68 |
| 6235 | CD1 | ILE | E | 16 | −0.707 | 69.252 | −19.676 | 1.00 | 89.68 |
| 6236 | C | ILE | E | 16 | −4.589 | 67.773 | −20.909 | 1.00 | 117.86 |
| 6237 | O | ILE | E | 16 | −4.302 | 66.678 | −21.390 | 1.00 | 117.86 |
| 6238 | N | PHE | E | 17 | −5.103 | 68.784 | −21.591 | 1.00 | 150.96 |
| 6239 | CA | PHE | E | 17 | −5.417 | 68.746 | −23.003 | 1.00 | 150.96 |
| 6240 | CB | PHE | E | 17 | −6.466 | 69.815 | −23.287 | 1.00 | 92.59 |
| 6241 | CG | PHE | E | 17 | −7.872 | 69.337 | −23.168 | 1.00 | 92.59 |
| 6242 | CD1 | PHE | E | 17 | −8.846 | 70.151 | −22.597 | 1.00 | 92.59 |
| 6243 | CD2 | PHE | E | 17 | −8.251 | 68.120 | −23.724 | 1.00 | 92.59 |
| 6244 | CE1 | PHE | E | 17 | −10.193 | 69.757 | −22.577 | 1.00 | 92.59 |
| 6245 | CE2 | PHE | E | 17 | −9.588 | 67.717 | −23.711 | 1.00 | 92.59 |
| 6246 | CZ | PHE | E | 17 | −10.567 | 68.546 | −23.141 | 1.00 | 92.59 |
| 6247 | C | PHE | E | 17 | −4.169 | 69.021 | −23.835 | 1.00 | 150.96 |
| 6248 | O | PHE | E | 17 | −3.184 | 69.562 | −23.333 | 1.00 | 150.96 |
| 6249 | N | LYS | E | 18 | −4.222 | 68.665 | −25.112 | 1.00 | 145.64 |
| 6250 | CA | LYS | E | 18 | −3.099 | 68.891 | −26.010 | 1.00 | 145.64 |
| 6251 | CB | LYS | E | 18 | −3.370 | 68.206 | −27.350 | 1.00 | 192.00 |
| 6252 | CG | LYS | E | 18 | −2.210 | 68.241 | −28.329 | 1.00 | 192.00 |
| 6253 | CD | LYS | E | 18 | −2.457 | 67.244 | −29.441 | 1.00 | 192.00 |
| 6254 | CE | LYS | E | 18 | −1.359 | 67.245 | −30.495 | 1.00 | 192.00 |
| 6255 | NZ | LYS | E | 18 | −1.218 | 68.567 | −31.165 | 1.00 | 192.00 |
| 6256 | C | LYS | E | 18 | −2.878 | 70.388 | −26.224 | 1.00 | 145.64 |
| 6257 | O | LYS | E | 18 | −3.814 | 71.129 | −26.517 | 1.00 | 145.64 |
| 6258 | N | GLY | E | 19 | −1.638 | 70.835 | −26.061 | 1.00 | 249.22 |
| 6259 | CA | GLY | E | 19 | −1.339 | 72.237 | −26.278 | 1.00 | 249.22 |
| 6260 | C | GLY | E | 19 | −1.364 | 73.144 | −25.065 | 1.00 | 249.22 |
| 6261 | O | GLY | E | 19 | −0.954 | 74.298 | −25.156 | 1.00 | 249.22 |
| 6262 | N | GLU | E | 20 | −1.837 | 72.648 | −23.930 | 1.00 | 144.61 |
| 6263 | CA | GLU | E | 20 | −1.882 | 73.481 | −22.732 | 1.00 | 144.61 |
| 6264 | CB | GLU | E | 20 | −2.930 | 72.929 | −21.759 | 1.00 | 147.82 |
| 6265 | CG | GLU | E | 20 | −4.288 | 72.690 | −22.425 | 1.00 | 147.82 |
| 6266 | CD | GLU | E | 20 | −5.371 | 72.257 | −21.449 | 1.00 | 147.82 |
| 6267 | OE1 | GLU | E | 20 | −5.166 | 71.259 | −20.729 | 1.00 | 147.82 |
| 6268 | OE2 | GLU | E | 20 | −6.435 | 72.910 | −21.411 | 1.00 | 147.82 |
| 6269 | C | GLU | E | 20 | −0.501 | 73.546 | −22.071 | 1.00 | 144.61 |
| 6270 | O | GLU | E | 20 | 0.412 | 72.815 | −22.466 | 1.00 | 144.61 |
| 6271 | N | ASN | E | 21 | −0.335 | 74.432 | −21.089 | 1.00 | 165.65 |
| 6272 | CA | ASN | E | 21 | 0.951 | 74.549 | −20.407 | 1.00 | 165.65 |
| 6273 | CB | ASN | E | 21 | 1.551 | 75.953 | −20.547 | 1.00 | 216.79 |
| 6274 | CG | ASN | E | 21 | 1.361 | 76.551 | −21.918 | 1.00 | 216.79 |
| 6275 | OD1 | ASN | E | 21 | 1.528 | 75.886 | −22.943 | 1.00 | 216.79 |
| 6276 | ND2 | ASN | E | 21 | 1.027 | 77.836 | −21.921 | 1.00 | 216.79 |
| 6277 | C | ASN | E | 21 | 0.837 | 74.248 | −18.917 | 1.00 | 165.65 |
| 6278 | O | ASN | E | 21 | −0.147 | 74.602 | −18.268 | 1.00 | 165.65 |
| 6279 | N | VAL | E | 22 | 1.868 | 73.608 | −18.380 | 1.00 | 160.77 |
| 6280 | CA | VAL | E | 22 | 1.912 | 73.265 | −16.968 | 1.00 | 160.77 |
| 6281 | CB | VAL | E | 22 | 1.497 | 71.820 | −16.737 | 1.00 | 158.92 |
| 6282 | CG1 | VAL | E | 22 | 2.481 | 70.881 | −17.426 | 1.00 | 158.92 |
| 6283 | CG2 | VAL | E | 22 | 1.448 | 71.543 | −15.256 | 1.00 | 158.92 |
| 6284 | C | VAL | E | 22 | 3.342 | 73.442 | −16.467 | 1.00 | 160.77 |
| 6285 | O | VAL | E | 22 | 4.306 | 73.287 | −17.229 | 1.00 | 160.77 |
| 6286 | N | THR | E | 23 | 3.478 | 73.743 | −15.180 | 1.00 | 119.61 |
| 6287 | CA | THR | E | 23 | 4.789 | 73.972 | −14.581 | 1.00 | 119.61 |
| 6288 | CB | THR | E | 23 | 4.862 | 75.412 | −14.037 | 1.00 | 249.32 |
| 6289 | OG1 | THR | E | 23 | 4.505 | 76.335 | −15.075 | 1.00 | 249.32 |
| 6290 | CG2 | THR | E | 23 | 6.255 | 75.728 | −13.537 | 1.00 | 249.32 |
| 6291 | C | THR | E | 23 | 5.089 | 73.004 | −13.434 | 1.00 | 119.61 |
| 6292 | O | THR | E | 23 | 4.291 | 72.881 | −12.515 | 1.00 | 119.61 |
| 6293 | N | LEU | E | 24 | 6.233 | 72.326 | −13.467 | 1.00 | 105.17 |
| 6294 | CA | LEU | E | 24 | 6.556 | 71.397 | −12.387 | 1.00 | 105.17 |
| 6295 | CB | LEU | E | 24 | 7.032 | 70.045 | −12.922 | 1.00 | 144.47 |
| 6296 | CG | LEU | E | 24 | 6.394 | 69.466 | −14.178 | 1.00 | 144.47 |
| 6297 | CD1 | LEU | E | 24 | 6.782 | 68.008 | −14.314 | 1.00 | 144.47 |
| 6298 | CD2 | LEU | E | 24 | 4.904 | 69.607 | −14.118 | 1.00 | 144.47 |
| 6299 | C | LEU | E | 24 | 7.635 | 71.944 | −11.482 | 1.00 | 105.17 |
| 6300 | O | LEU | E | 24 | 8.814 | 71.943 | −11.821 | 1.00 | 105.17 |
| 6301 | N | THR | E | 25 | 7.238 | 72.386 | −10.306 | 1.00 | 95.95 |
| 6302 | CA | THR | E | 25 | 8.206 | 72.926 | −9.380 | 1.00 | 95.95 |
| 6303 | CB | THR | E | 25 | 7.552 | 74.012 | −8.528 | 1.00 | 178.12 |
| 6304 | OG1 | THR | E | 25 | 6.961 | 74.986 | −9.397 | 1.00 | 178.12 |
| 6305 | CG2 | THR | E | 25 | 8.578 | 74.689 | −7.639 | 1.00 | 178.12 |
| 6306 | C | THR | E | 25 | 8.786 | 71.833 | −8.486 | 1.00 | 95.95 |
| 6307 | O | THR | E | 25 | 8.062 | 70.964 | −8.004 | 1.00 | 95.95 |
| 6308 | N | CYS | E | 26 | 10.098 | 71.858 | −8.279 | 1.00 | 175.10 |
| 6309 | CA | CYS | E | 26 | 10.709 | 70.859 | −7.421 | 1.00 | 175.10 |
| 6310 | C | CYS | E | 26 | 10.598 | 71.335 | −5.991 | 1.00 | 175.10 |
| 6311 | O | CYS | E | 26 | 10.769 | 72.518 | −5.698 | 1.00 | 175.10 |
| 6312 | CB | CYS | E | 26 | 12.178 | 70.637 | −7.771 | 1.00 | 230.60 |
| 6313 | SG | CYS | E | 26 | 12.906 | 69.220 | −6.890 | 1.00 | 230.60 |
| 6314 | N | ASN | E | 27 | 10.293 | 70.392 | −5.114 | 1.00 | 159.04 |
| 6315 | CA | ASN | E | 27 | 10.141 | 70.641 | −3.696 | 1.00 | 159.04 |
| 6316 | CB | ASN | E | 27 | 10.980 | 69.628 | −2.940 | 1.00 | 118.85 |
| 6317 | CG | ASN | E | 27 | 10.546 | 69.486 | −1.511 | 1.00 | 118.85 |
| 6318 | OD1 | ASN | E | 27 | 9.351 | 69.537 | −1.211 | 1.00 | 118.85 |
| 6319 | ND2 | ASN | E | 27 | 11.506 | 69.298 | −0.613 | 1.00 | 118.85 |
| 6320 | C | ASN | E | 27 | 10.502 | 72.054 | −3.237 | 1.00 | 159.04 |
| 6321 | O | ASN | E | 27 | 11.646 | 72.331 | −2.882 | 1.00 | 159.04 |
| 6322 | N | GLY | E | 28 | 9.516 | 72.944 | −3.238 | 1.00 | 225.84 |
| 6323 | CA | GLY | E | 28 | 9.750 | 74.315 | −2.824 | 1.00 | 225.84 |
| 6324 | C | GLY | E | 28 | 8.487 | 75.101 | −3.091 | 1.00 | 225.84 |
| 6325 | O | GLY | E | 28 | 8.021 | 75.153 | −4.227 | 1.00 | 225.84 |
| 6326 | N | ASN | E | 29 | 7.929 | 75.722 | −2.059 | 1.00 | 249.43 |
| 6327 | CA | ASN | E | 29 | 6.693 | 76.466 | −2.228 | 1.00 | 249.43 |
| 6328 | CB | ASN | E | 29 | 6.026 | 76.682 | −0.870 | 1.00 | 249.43 |
| 6329 | CG | ASN | E | 29 | 4.607 | 77.193 | −0.996 | 1.00 | 249.43 |
| 6330 | OD1 | ASN | E | 29 | 3.999 | 77.145 | −2.066 | 1.00 | 249.43 |
| 6331 | ND2 | ASN | E | 29 | 4.067 | 77.674 | 0.104 | 1.00 | 249.43 |
| 6332 | C | ASN | E | 29 | 6.820 | 77.799 | −2.962 | 1.00 | 249.43 |
| 6333 | O | ASN | E | 29 | 6.084 | 78.052 | −3.920 | 1.00 | 249.43 |
| 6334 | N | ASN | E | 30 | 7.746 | 78.649 | −2.530 | 1.00 | 249.58 |
| 6335 | CA | ASN | E | 30 | 7.903 | 79.952 | −3.169 | 1.00 | 249.58 |
| 6336 | CB | ASN | E | 30 | 7.420 | 81.060 | −2.229 | 1.00 | 249.27 |
| 6337 | CG | ASN | E | 30 | 5.941 | 80.965 | −1.929 | 1.00 | 249.27 |
| 6338 | OD1 | ASN | E | 30 | 5.532 | 80.984 | −0.770 | 1.00 | 249.27 |
| 6339 | ND2 | ASN | E | 30 | 5.128 | 80.865 | −2.974 | 1.00 | 249.27 |
| 6340 | C | ASN | E | 30 | 9.313 | 80.279 | −3.633 | 1.00 | 249.58 |
| 6341 | O | ASN | E | 30 | 9.589 | 80.313 | −4.833 | 1.00 | 249.58 |
| 6342 | N | PHE | E | 31 | 10.206 | 80.526 | −2.682 | 1.00 | 249.39 |
| 6343 | CA | PHE | E | 31 | 11.567 | 80.882 | −3.038 | 1.00 | 249.39 |
| 6344 | CB | PHE | E | 31 | 11.939 | 82.212 | −2.368 | 1.00 | 249.51 |
| 6345 | CG | PHE | E | 31 | 10.976 | 83.336 | −2.673 | 1.00 | 249.51 |
| 6346 | CD1 | PHE | E | 31 | 9.760 | 83.431 | −2.003 | 1.00 | 249.51 |
| 6347 | CD2 | PHE | E | 31 | 11.275 | 84.285 | −3.649 | 1.00 | 249.51 |
| 6348 | CE1 | PHE | E | 31 | 8.855 | 84.455 | −2.294 | 1.00 | 249.51 |
| 6349 | CE2 | PHE | E | 31 | 10.378 | 85.313 | −3.949 | 1.00 | 249.51 |
| 6350 | CZ | PHE | E | 31 | 9.164 | 85.396 | −3.270 | 1.00 | 249.51 |
| 6351 | C | PHE | E | 31 | 12.602 | 79.806 | −2.729 | 1.00 | 249.39 |
| 6352 | O | PHE | E | 31 | 12.696 | 79.305 | −1.605 | 1.00 | 249.39 |
| 6353 | N | PHE | E | 32 | 13.374 | 79.462 | −3.760 | 1.00 | 249.36 |
| 6354 | CA | PHE | E | 32 | 14.421 | 78.447 | −3.678 | 1.00 | 249.36 |
| 6355 | CB | PHE | E | 32 | 14.088 | 77.296 | −4.623 | 1.00 | 231.13 |
| 6356 | CG | PHE | E | 32 | 14.910 | 76.075 | −4.386 | 1.00 | 231.13 |
| 6357 | CD1 | PHE | E | 32 | 14.771 | 75.366 | −3.200 | 1.00 | 231.13 |
| 6358 | CD2 | PHE | E | 32 | 15.844 | 75.644 | −5.325 | 1.00 | 231.13 |
| 6359 | CE1 | PHE | E | 32 | 15.547 | 74.245 | −2.944 | 1.00 | 231.13 |
| 6360 | CE2 | PHE | E | 32 | 16.628 | 74.520 | −5.079 | 1.00 | 231.13 |
| 6361 | CZ | PHE | E | 32 | 16.477 | 73.819 | −3.881 | 1.00 | 231.13 |
| 6362 | C | PHE | E | 32 | 15.779 | 79.040 | −4.063 | 1.00 | 249.36 |
| 6363 | O | PHE | E | 32 | 15.849 | 80.171 | −4.540 | 1.00 | 249.36 |
| 6364 | N | GLU | E | 33 | 16.857 | 78.277 | −3.876 | 1.00 | 249.65 |
| 6365 | CA | GLU | E | 33 | 18.190 | 78.784 | −4.212 | 1.00 | 249.65 |
| 6366 | CB | GLU | E | 33 | 19.035 | 78.993 | −2.958 | 1.00 | 249.51 |
| 6367 | CG | GLU | E | 33 | 20.347 | 79.715 | −3.258 | 1.00 | 249.51 |
| 6368 | CD | GLU | E | 33 | 20.106 | 81.122 | −3.769 | 1.00 | 249.51 |
| 6369 | OE1 | GLU | E | 33 | 19.170 | 81.769 | −3.253 | 1.00 | 249.51 |
| 6370 | OE2 | GLU | E | 33 | 20.849 | 81.591 | −4.659 | 1.00 | 249.51 |
| 6371 | C | GLU | E | 33 | 19.038 | 77.975 | −5.180 | 1.00 | 249.65 |
| 6372 | O | GLU | E | 33 | 19.533 | 78.510 | −6.173 | 1.00 | 249.65 |
| 6373 | N | VAL | E | 34 | 19.242 | 76.699 | −4.873 | 1.00 | 249.34 |
| 6374 | CA | VAL | E | 34 | 20.073 | 75.864 | −5.721 | 1.00 | 249.34 |
| 6375 | CB | VAL | E | 34 | 20.055 | 74.399 | −5.244 | 1.00 | 177.29 |
| 6376 | CG1 | VAL | E | 34 | 20.927 | 73.540 | −6.146 | 1.00 | 177.29 |
| 6377 | CG2 | VAL | E | 34 | 20.562 | 74.325 | −3.815 | 1.00 | 177.29 |
| 6378 | C | VAL | E | 34 | 19.694 | 75.933 | −7.196 | 1.00 | 249.34 |
| 6379 | O | VAL | E | 34 | 18.530 | 76.142 | −7.555 | 1.00 | 249.34 |
| 6380 | N | SER | E | 35 | 20.705 | 75.782 | −8.040 | 1.00 | 249.49 |
| 6381 | CA | SER | E | 35 | 20.523 | 75.809 | −9.479 | 1.00 | 249.49 |
| 6382 | CB | SER | E | 35 | 21.517 | 76.779 | −10.122 | 1.00 | 217.44 |
| 6383 | OG | SER | E | 35 | 22.845 | 76.294 | −10.006 | 1.00 | 217.44 |
| 6384 | C | SER | E | 35 | 20.763 | 74.397 | −10.001 | 1.00 | 249.49 |
| 6385 | O | SER | E | 35 | 20.658 | 74.144 | −11.199 | 1.00 | 249.49 |
| 6386 | N | SER | E | 36 | 21.094 | 73.480 | −9.092 | 1.00 | 249.36 |
| 6387 | CA | SER | E | 36 | 21.335 | 72.089 | −9.464 | 1.00 | 249.36 |
| 6388 | CB | SER | E | 36 | 22.586 | 71.540 | −8.769 | 1.00 | 172.90 |
| 6389 | OG | SER | E | 36 | 22.371 | 71.370 | −7.379 | 1.00 | 172.90 |
| 6390 | C | SER | E | 36 | 20.128 | 71.242 | −9.085 | 1.00 | 249.36 |
| 6391 | O | SER | E | 36 | 20.020 | 70.741 | −7.964 | 1.00 | 249.36 |
| 6392 | N | THR | E | 37 | 19.214 | 71.104 | −10.036 | 1.00 | 210.16 |
| 6393 | CA | THR | E | 37 | 18.007 | 70.321 | −9.849 | 1.00 | 210.16 |
| 6394 | CB | THR | E | 37 | 16.754 | 71.225 | −9.905 | 1.00 | 202.55 |
| 6395 | OG1 | THR | E | 37 | 16.859 | 72.253 | −8.913 | 1.00 | 202.55 |
| 6396 | CG2 | THR | E | 37 | 15.499 | 70.414 | −9.647 | 1.00 | 202.55 |
| 6397 | C | THR | E | 37 | 17.982 | 69.325 | −11.000 | 1.00 | 210.16 |
| 6398 | O | THR | E | 37 | 18.352 | 69.662 | −12.126 | 1.00 | 210.16 |
| 6399 | N | LYS | E | 38 | 17.565 | 68.098 | −10.718 | 1.00 | 223.06 |
| 6400 | CA | LYS | E | 38 | 17.517 | 67.070 | −11.749 | 1.00 | 223.06 |
| 6401 | CB | LYS | E | 38 | 18.234 | 65.818 | −11.256 | 1.00 | 249.17 |
| 6402 | CG | LYS | E | 38 | 19.660 | 66.069 | −10.828 | 1.00 | 249.17 |
| 6403 | CD | LYS | E | 38 | 20.313 | 64.794 | −10.338 | 1.00 | 249.17 |
| 6404 | CE | LYS | E | 38 | 21.769 | 65.032 | −9.996 | 1.00 | 249.17 |
| 6405 | NZ | LYS | E | 38 | 22.436 | 63.783 | −9.543 | 1.00 | 249.17 |
| 6406 | C | LYS | E | 38 | 16.086 | 66.711 | −12.133 | 1.00 | 223.06 |
| 6407 | O | LYS | E | 38 | 15.204 | 66.678 | −11.281 | 1.00 | 223.06 |
| 6408 | N | TRP | E | 39 | 15.858 | 66.451 | −13.418 | 1.00 | 178.64 |
| 6409 | CA | TRP | E | 39 | 14.530 | 66.068 | −13.895 | 1.00 | 178.64 |
| 6410 | CB | TRP | E | 39 | 13.911 | 67.160 | −14.768 | 1.00 | 178.88 |
| 6411 | CG | TRP | E | 39 | 13.622 | 68.431 | −14.049 | 1.00 | 178.88 |
| 6412 | CD2 | TRP | E | 39 | 12.634 | 68.651 | −13.034 | 1.00 | 178.88 |
| 6413 | CE2 | TRP | E | 39 | 12.721 | 70.008 | −12.652 | 1.00 | 178.88 |
| 6414 | CE3 | TRP | E | 39 | 11.685 | 67.832 | −12.407 | 1.00 | 178.88 |
| 6415 | CD1 | TRP | E | 39 | 14.249 | 69.627 | −14.235 | 1.00 | 178.88 |
| 6416 | NE1 | TRP | E | 39 | 13.712 | 70.580 | −13.400 | 1.00 | 178.88 |
| 6417 | CZ2 | TRP | E | 39 | 11.896 | 70.564 | −11.678 | 1.00 | 178.88 |
| 6418 | CZ3 | TRP | E | 39 | 10.865 | 68.390 | −11.436 | 1.00 | 178.88 |
| 6419 | CH2 | TRP | E | 39 | 10.978 | 69.744 | −11.081 | 1.00 | 178.88 |
| 6420 | C | TRP | E | 39 | 14.641 | 64.796 | −14.710 | 1.00 | 178.64 |
| 6421 | O | TRP | E | 39 | 15.495 | 64.687 | −15.582 | 1.00 | 178.64 |
| 6422 | N | PHE | E | 40 | 13.771 | 63.838 | −14.432 | 1.00 | 223.76 |
| 6423 | CA | PHE | E | 40 | 13.811 | 62.585 | −15.159 | 1.00 | 223.76 |
| 6424 | CB | PHE | E | 40 | 14.209 | 61.445 | −14.223 | 1.00 | 188.15 |
| 6425 | CG | PHE | E | 40 | 15.514 | 61.660 | −13.529 | 1.00 | 188.15 |
| 6426 | CD1 | PHE | E | 40 | 15.592 | 62.472 | −12.407 | 1.00 | 188.15 |
| 6427 | CD2 | PHE | E | 40 | 16.663 | 61.029 | −13.984 | 1.00 | 188.15 |
| 6428 | CE1 | PHE | E | 40 | 16.797 | 62.656 | −11.746 | 1.00 | 188.15 |
| 6429 | CE2 | PHE | E | 40 | 17.875 | 61.204 | −13.333 | 1.00 | 188.15 |
| 6430 | CZ | PHE | E | 40 | 17.942 | 62.020 | −12.207 | 1.00 | 188.15 |
| 6431 | C | PHE | E | 40 | 12.490 | 62.235 | −15.834 | 1.00 | 223.76 |
| 6432 | O | PHE | E | 40 | 11.665 | 61.521 | −15.266 | 1.00 | 223.76 |
| 6433 | N | HIS | E | 41 | 12.294 | 62.737 | −17.048 | 1.00 | 123.84 |
| 6434 | CA | HIS | E | 41 | 11.080 | 62.448 | −17.801 | 1.00 | 123.84 |
| 6435 | CB | HIS | E | 41 | 10.940 | 63.454 | −18.937 | 1.00 | 124.43 |
| 6436 | CG | HIS | E | 41 | 9.749 | 63.222 | −19.801 | 1.00 | 124.43 |
| 6437 | CD2 | HIS | E | 41 | 9.597 | 63.290 | −21.144 | 1.00 | 124.43 |
| 6438 | ND1 | HIS | E | 41 | 8.510 | 62.907 | −19.289 | 1.00 | 124.43 |
| 6439 | CE1 | HIS | E | 41 | 7.645 | 62.789 | −20.278 | 1.00 | 124.43 |
| 6440 | NE2 | HIS | E | 41 | 8.280 | 63.016 | −21.415 | 1.00 | 124.43 |
| 6441 | C | HIS | E | 41 | 11.136 | 61.013 | −18.349 | 1.00 | 123.84 |
| 6442 | O | HIS | E | 41 | 11.924 | 60.715 | −19.243 | 1.00 | 123.84 |
| 6443 | N | ASN | E | 42 | 10.298 | 60.132 | −17.809 | 1.00 | 190.21 |
| 6444 | CA | ASN | E | 42 | 10.269 | 58.717 | −18.206 | 1.00 | 190.21 |
| 6445 | CB | ASN | E | 42 | 10.027 | 58.550 | −19.720 | 1.00 | 194.75 |
| 6446 | CG | ASN | E | 42 | 8.588 | 58.839 | −20.123 | 1.00 | 194.75 |
| 6447 | OD1 | ASN | E | 42 | 8.009 | 59.813 | −19.653 | 1.00 | 194.75 |
| 6448 | ND2 | ASN | E | 42 | 8.017 | 58.019 | −21.005 | 1.00 | 194.75 |
| 6449 | C | ASN | E | 42 | 11.593 | 58.050 | −17.826 | 1.00 | 190.21 |
| 6450 | O | ASN | E | 42 | 12.003 | 57.072 | −18.446 | 1.00 | 190.21 |
| 6451 | N | GLY | E | 43 | 12.263 | 58.580 | −16.806 | 1.00 | 203.91 |
| 6452 | CA | GLY | E | 43 | 13.533 | 58.010 | −16.386 | 1.00 | 203.91 |
| 6453 | C | GLY | E | 43 | 14.734 | 58.697 | −17.020 | 1.00 | 203.91 |
| 6454 | O | GLY | E | 43 | 15.758 | 58.901 | −16.364 | 1.00 | 203.91 |
| 6455 | N | SER | E | 44 | 14.609 | 59.053 | −18.297 | 1.00 | 245.20 |
| 6456 | CA | SER | E | 44 | 15.683 | 59.723 | −19.030 | 1.00 | 245.20 |
| 6457 | CB | SER | E | 44 | 15.312 | 59.846 | −20.512 | 1.00 | 220.02 |
| 6458 | OG | SER | E | 44 | 14.940 | 58.591 | −21.055 | 1.00 | 220.02 |
| 6459 | C | SER | E | 44 | 15.929 | 61.114 | −18.452 | 1.00 | 245.20 |
| 6460 | O | SER | E | 44 | 14.999 | 61.907 | −18.326 | 1.00 | 245.20 |
| 6461 | N | LEU | E | 45 | 17.177 | 61.412 | −18.101 | 1.00 | 174.49 |
| 6462 | CA | LEU | E | 45 | 17.519 | 62.718 | −17.541 | 1.00 | 174.49 |
| 6463 | CB | LEU | E | 45 | 19.028 | 62.804 | −17.280 | 1.00 | 249.38 |
| 6464 | CG | LEU | E | 45 | 19.550 | 64.104 | −16.660 | 1.00 | 249.38 |
| 6465 | CD1 | LEU | E | 45 | 18.785 | 64.404 | −15.381 | 1.00 | 249.38 |
| 6466 | CD2 | LEU | E | 45 | 21.043 | 63.982 | −16.375 | 1.00 | 249.38 |
| 6467 | C | LEU | E | 45 | 17.095 | 63.834 | −18.498 | 1.00 | 174.49 |
| 6468 | O | LEU | E | 45 | 17.140 | 63.672 | −19.717 | 1.00 | 174.49 |
| 6469 | N | SER | E | 46 | 16.673 | 64.965 | −17.945 | 1.00 | 153.34 |
| 6470 | CA | SER | E | 46 | 16.247 | 66.094 | −18.766 | 1.00 | 153.34 |
| 6471 | CB | SER | E | 46 | 15.016 | 66.766 | −18.148 | 1.00 | 249.33 |
| 6472 | OG | SER | E | 46 | 14.541 | 67.822 | −18.971 | 1.00 | 249.33 |
| 6473 | C | SER | E | 46 | 17.394 | 67.088 | −18.845 | 1.00 | 153.34 |
| 6474 | O | SER | E | 46 | 18.345 | 66.994 | −18.072 | 1.00 | 153.34 |
| 6475 | N | GLU | E | 47 | 17.310 | 68.043 | −19.768 | 1.00 | 221.85 |
| 6476 | CA | GLU | E | 47 | 18.371 | 69.035 | −19.903 | 1.00 | 221.85 |
| 6477 | CB | GLU | E | 47 | 18.589 | 69.401 | −21.384 | 1.00 | 249.45 |
| 6478 | CG | GLU | E | 47 | 18.515 | 68.232 | −22.369 | 1.00 | 249.45 |
| 6479 | CD | GLU | E | 47 | 18.351 | 68.687 | −23.823 | 1.00 | 249.45 |
| 6480 | OE1 | GLU | E | 47 | 17.207 | 68.973 | −24.239 | 1.00 | 249.45 |
| 6481 | OE2 | GLU | E | 47 | 19.372 | 68.774 | −24.540 | 1.00 | 249.45 |
| 6482 | C | GLU | E | 47 | 18.128 | 70.317 | −19.081 | 1.00 | 221.85 |
| 6483 | O | GLU | E | 47 | 18.974 | 71.207 | −19.091 | 1.00 | 221.85 |
| 6484 | N | GLU | E | 48 | 16.993 | 70.438 | −18.387 | 1.00 | 204.14 |
| 6485 | CA | GLU | E | 48 | 16.775 | 71.643 | −17.573 | 1.00 | 204.14 |
| 6486 | CB | GLU | E | 48 | 15.275 | 71.939 | −17.355 | 1.00 | 206.52 |
| 6487 | CG | GLU | E | 48 | 14.973 | 73.087 | −16.352 | 1.00 | 206.52 |
| 6488 | CD | GLU | E | 48 | 15.418 | 74.469 | −16.823 | 1.00 | 206.52 |
| 6489 | OE1 | GLU | E | 48 | 14.812 | 74.995 | −17.778 | 1.00 | 206.52 |
| 6490 | OE2 | GLU | E | 48 | 16.370 | 75.032 | −16.236 | 1.00 | 206.52 |
| 6491 | C | GLU | E | 48 | 17.471 | 71.443 | −16.221 | 1.00 | 204.14 |
| 6492 | O | GLU | E | 48 | 17.724 | 70.311 | −15.798 | 1.00 | 204.14 |
| 6493 | N | THR | E | 49 | 17.803 | 72.545 | −15.556 | 1.00 | 206.12 |
| 6494 | CA | THR | E | 49 | 18.472 | 72.476 | −14.266 | 1.00 | 206.12 |
| 6495 | CB | THR | E | 49 | 19.947 | 72.928 | −14.386 | 1.00 | 224.40 |
| 6496 | OG1 | THR | E | 49 | 20.006 | 74.247 | −14.942 | 1.00 | 224.40 |
| 6497 | CG2 | THR | E | 49 | 20.721 | 71.972 | −15.288 | 1.00 | 224.40 |
| 6498 | C | THR | E | 49 | 17.747 | 73.335 | −13.233 | 1.00 | 206.12 |
| 6499 | O | THR | E | 49 | 17.781 | 73.043 | −12.035 | 1.00 | 206.12 |
| 6500 | N | ASN | E | 50 | 17.081 | 74.388 | −13.702 | 1.00 | 231.11 |
| 6501 | CA | ASN | E | 50 | 16.330 | 75.272 | −12.819 | 1.00 | 231.11 |
| 6502 | CB | ASN | E | 50 | 15.602 | 76.349 | −13.640 | 1.00 | 176.85 |
| 6503 | CG | ASN | E | 50 | 15.085 | 77.491 | −12.783 | 1.00 | 176.85 |
| 6504 | OD1 | ASN | E | 50 | 14.962 | 77.346 | −11.568 | 1.00 | 176.85 |
| 6505 | ND2 | ASN | E | 50 | 14.770 | 78.622 | −13.410 | 1.00 | 176.85 |
| 6506 | C | ASN | E | 50 | 15.316 | 74.396 | −12.076 | 1.00 | 231.11 |
| 6507 | O | ASN | E | 50 | 14.884 | 73.368 | −12.597 | 1.00 | 231.11 |
| 6508 | N | SER | E | 51 | 14.942 | 74.792 | −10.863 | 1.00 | 235.89 |
| 6509 | CA | SER | E | 51 | 13.985 | 74.015 | −10.077 | 1.00 | 235.89 |
| 6510 | CB | SER | E | 51 | 13.895 | 74.561 | −8.645 | 1.00 | 153.05 |
| 6511 | OG | SER | E | 51 | 13.254 | 75.826 | −8.609 | 1.00 | 153.05 |
| 6512 | C | SER | E | 51 | 12.587 | 73.995 | −10.696 | 1.00 | 235.89 |
| 6513 | O | SER | E | 51 | 11.765 | 73.145 | −10.358 | 1.00 | 235.89 |
| 6514 | N | SER | E | 52 | 12.314 | 74.932 | −11.597 | 1.00 | 154.90 |
| 6515 | CA | SER | E | 52 | 11.009 | 74.997 | −12.245 | 1.00 | 154.90 |
| 6516 | CB | SER | E | 52 | 10.435 | 76.415 | −12.157 | 1.00 | 199.68 |
| 6517 | OG | SER | E | 52 | 10.195 | 76.786 | −10.809 | 1.00 | 199.68 |
| 6518 | C | SER | E | 52 | 11.109 | 74.569 | −13.700 | 1.00 | 154.90 |
| 6519 | O | SER | E | 52 | 11.656 | 75.288 | −14.538 | 1.00 | 154.90 |
| 6520 | N | LEU | E | 53 | 10.582 | 73.385 | −13.985 | 1.00 | 130.79 |
| 6521 | CA | LEU | E | 53 | 10.590 | 72.827 | −15.332 | 1.00 | 130.79 |
| 6522 | CB | LEU | E | 53 | 10.833 | 71.315 | −15.264 | 1.00 | 134.25 |
| 6523 | CG | LEU | E | 53 | 10.394 | 70.457 | −16.457 | 1.00 | 134.25 |
| 6524 | CD1 | LEU | E | 53 | 10.802 | 71.095 | −17.779 | 1.00 | 134.25 |
| 6525 | CD2 | LEU | E | 53 | 10.999 | 69.065 | −16.304 | 1.00 | 134.25 |
| 6526 | C | LEU | E | 53 | 9.271 | 73.112 | −16.044 | 1.00 | 130.79 |
| 6527 | O | LEU | E | 53 | 8.279 | 72.435 | −15.810 | 1.00 | 130.79 |
| 6528 | N | ASN | E | 54 | 9.258 | 74.109 | −16.920 | 1.00 | 200.88 |
| 6529 | CA | ASN | E | 54 | 8.031 | 74.440 | −17.632 | 1.00 | 200.88 |
| 6530 | CB | ASN | E | 54 | 8.095 | 75.864 | −18.181 | 1.00 | 249.13 |
| 6531 | CG | ASN | E | 54 | 7.990 | 76.907 | −17.096 | 1.00 | 249.13 |
| 6532 | OD1 | ASN | E | 54 | 7.029 | 76.923 | −16.328 | 1.00 | 249.13 |
| 6533 | ND2 | ASN | E | 54 | 8.975 | 77.790 | −17.026 | 1.00 | 249.13 |
| 6534 | C | ASN | E | 54 | 7.719 | 73.486 | −18.771 | 1.00 | 200.88 |
| 6535 | O | ASN | E | 54 | 8.589 | 72.769 | −19.265 | 1.00 | 200.88 |
| 6536 | N | ILE | E | 55 | 6.453 | 73.481 | −19.168 | 1.00 | 204.06 |
| 6537 | CA | ILE | E | 55 | 5.985 | 72.657 | −20.269 | 1.00 | 204.06 |
| 6538 | CB | ILE | E | 55 | 5.212 | 71.417 | −19.770 | 1.00 | 202.84 |
| 6539 | CG2 | ILE | E | 55 | 4.367 | 70.839 | −20.896 | 1.00 | 202.84 |
| 6540 | CG1 | ILE | E | 55 | 6.205 | 70.376 | −19.246 | 1.00 | 202.84 |
| 6541 | CD1 | ILE | E | 55 | 5.569 | 69.111 | −18.716 | 1.00 | 202.84 |
| 6542 | C | ILE | E | 55 | 5.065 | 73.547 | −21.080 | 1.00 | 204.06 |
| 6543 | O | ILE | E | 55 | 4.086 | 74.074 | −20.559 | 1.00 | 204.06 |
| 6544 | N | VAL | E | 56 | 5.395 | 73.739 | −22.349 | 1.00 | 244.52 |
| 6545 | CA | VAL | E | 56 | 4.580 | 74.585 | −23.202 | 1.00 | 244.52 |
| 6546 | CB | VAL | E | 56 | 5.458 | 75.494 | −24.072 | 1.00 | 219.78 |
| 6547 | CG1 | VAL | E | 56 | 4.629 | 76.643 | −24.618 | 1.00 | 219.78 |
| 6548 | CG2 | VAL | E | 56 | 6.622 | 76.026 | −23.246 | 1.00 | 219.78 |
| 6549 | C | VAL | E | 56 | 3.711 | 73.696 | −24.073 | 1.00 | 244.52 |
| 6550 | O | VAL | E | 56 | 3.545 | 72.522 | −23.758 | 1.00 | 244.52 |
| 6551 | N | ASN | E | 57 | 3.160 | 74.247 | −25.152 | 1.00 | 153.88 |
| 6552 | CA | ASN | E | 57 | 2.290 | 73.486 | −26.047 | 1.00 | 153.88 |
| 6553 | CB | ASN | E | 57 | 2.564 | 73.854 | −27.506 | 1.00 | 249.23 |
| 6554 | CG | ASN | E | 57 | 2.105 | 75.254 | −27.843 | 1.00 | 249.23 |
| 6555 | OD1 | ASN | E | 57 | 0.954 | 75.617 | −27.601 | 1.00 | 249.23 |
| 6556 | ND2 | ASN | E | 57 | 3.003 | 76.051 | −28.404 | 1.00 | 249.23 |
| 6557 | C | ASN | E | 57 | 2.438 | 71.983 | −25.847 | 1.00 | 153.88 |
| 6558 | O | ASN | E | 57 | 3.263 | 71.323 | −26.489 | 1.00 | 153.88 |
| 6559 | N | ALA | E | 58 | 1.624 | 71.458 | −24.936 | 1.00 | 183.15 |
| 6560 | CA | ALA | E | 58 | 1.638 | 70.047 | −24.587 | 1.00 | 183.15 |
| 6561 | CB | ALA | E | 58 | 0.552 | 69.763 | −23.565 | 1.00 | 127.72 |
| 6562 | C | ALA | E | 58 | 1.492 | 69.103 | −25.766 | 1.00 | 183.15 |
| 6563 | O | ALA | E | 58 | 0.486 | 69.115 | −26.474 | 1.00 | 183.15 |
| 6564 | N | LYS | E | 59 | 2.510 | 68.281 | −25.968 | 1.00 | 111.87 |
| 6565 | CA | LYS | E | 59 | 2.495 | 67.293 | −27.035 | 1.00 | 111.87 |
| 6566 | CB | LYS | E | 59 | 3.816 | 67.338 | −27.815 | 1.00 | 249.40 |
| 6567 | CG | LYS | E | 59 | 4.115 | 68.702 | −28.436 | 1.00 | 249.40 |
| 6568 | CD | LYS | E | 59 | 5.489 | 68.753 | −29.090 | 1.00 | 249.40 |
| 6569 | CE | LYS | E | 59 | 5.764 | 70.129 | −29.691 | 1.00 | 249.40 |
| 6570 | NZ | LYS | E | 59 | 7.117 | 70.219 | −30.310 | 1.00 | 249.40 |
| 6571 | C | LYS | E | 59 | 2.319 | 65.942 | −26.334 | 1.00 | 111.87 |
| 6572 | O | LYS | E | 59 | 2.824 | 65.746 | −25.226 | 1.00 | 111.87 |
| 6573 | N | PHE | E | 60 | 1.597 | 65.020 | −26.960 | 1.00 | 223.03 |
| 6574 | CA | PHE | E | 60 | 1.368 | 63.703 | −26.366 | 1.00 | 223.03 |
| 6575 | CB | PHE | E | 60 | 0.846 | 62.744 | −27.427 | 1.00 | 249.06 |
| 6576 | CG | PHE | E | 60 | −0.496 | 63.120 | −27.953 | 1.00 | 249.06 |
| 6577 | CD1 | PHE | E | 60 | −0.876 | 62.774 | −29.237 | 1.00 | 249.06 |
| 6578 | CD2 | PHE | E | 60 | −1.390 | 63.823 | −27.155 | 1.00 | 249.06 |
| 6579 | CE1 | PHE | E | 60 | −2.124 | 63.119 | −29.727 | 1.00 | 249.06 |
| 6580 | CE2 | PHE | E | 60 | −2.640 | 64.176 | −27.633 | 1.00 | 249.06 |
| 6581 | CZ | PHE | E | 60 | −3.010 | 63.822 | −28.922 | 1.00 | 249.06 |
| 6582 | C | PHE | E | 60 | 2.610 | 63.115 | −25.720 | 1.00 | 223.03 |
| 6583 | O | PHE | E | 60 | 2.520 | 62.404 | −24.721 | 1.00 | 223.03 |
| 6584 | N | GLU | E | 61 | 3.771 | 63.417 | −26.296 | 1.00 | 190.77 |
| 6585 | CA | GLU | E | 61 | 5.044 | 62.917 | −25.783 | 1.00 | 190.77 |
| 6586 | CB | GLU | E | 61 | 6.196 | 63.299 | −26.718 | 1.00 | 249.27 |
| 6587 | CG | GLU | E | 61 | 6.096 | 62.728 | −28.116 | 1.00 | 249.27 |
| 6588 | CD | GLU | E | 61 | 4.851 | 63.190 | −28.838 | 1.00 | 249.27 |
| 6589 | OE1 | GLU | E | 61 | 4.635 | 64.418 | −28.924 | 1.00 | 249.27 |
| 6590 | OE2 | GLU | E | 61 | 4.090 | 62.327 | −29.320 | 1.00 | 249.27 |
| 6591 | C | GLU | E | 61 | 5.357 | 63.449 | −24.395 | 1.00 | 190.77 |
| 6592 | O | GLU | E | 61 | 6.140 | 62.842 | −23.663 | 1.00 | 190.77 |
| 6593 | N | ASP | E | 62 | 4.765 | 64.588 | −24.040 | 1.00 | 156.70 |
| 6594 | CA | ASP | E | 62 | 5.006 | 65.174 | −22.727 | 1.00 | 156.70 |
| 6595 | CB | ASP | E | 62 | 4.489 | 66.613 | −22.678 | 1.00 | 165.21 |
| 6596 | CG | ASP | E | 62 | 5.062 | 67.477 | −23.792 | 1.00 | 165.21 |
| 6597 | OD1 | ASP | E | 62 | 6.251 | 67.299 | −24.133 | 1.00 | 165.21 |
| 6598 | OD2 | ASP | E | 62 | 4.329 | 68.341 | −24.320 | 1.00 | 165.21 |
| 6599 | C | ASP | E | 62 | 4.341 | 64.324 | −21.643 | 1.00 | 156.70 |
| 6600 | O | ASP | E | 62 | 4.711 | 64.394 | −20.470 | 1.00 | 158.70 |
| 6601 | N | SER | E | 63 | 3.358 | 63.523 | −22.045 | 1.00 | 140.02 |
| 6602 | CA | SER | E | 63 | 2.672 | 62.635 | −21.118 | 1.00 | 140.02 |
| 6603 | CB | SER | E | 63 | 1.618 | 61.796 | −21.856 | 1.00 | 116.21 |
| 6604 | OG | SER | E | 63 | 0.557 | 62.589 | −22.368 | 1.00 | 116.21 |
| 6605 | C | SER | E | 63 | 3.744 | 61.710 | −20.557 | 1.00 | 140.02 |
| 6606 | O | SER | E | 63 | 4.509 | 61.128 | −21.315 | 1.00 | 140.02 |
| 6607 | N | GLY | E | 64 | 3.818 | 61.572 | −19.243 | 1.00 | 94.90 |
| 6608 | CA | GLY | E | 64 | 4.835 | 60.694 | −18.698 | 1.00 | 94.90 |
| 6609 | C | GLY | E | 64 | 5.050 | 60.749 | −17.195 | 1.00 | 94.90 |
| 6610 | O | GLY | E | 64 | 4.252 | 61.333 | −16.462 | 1.00 | 94.90 |
| 6611 | N | GLU | E | 65 | 6.127 | 60.121 | −16.737 | 1.00 | 137.73 |
| 6612 | CA | GLU | E | 65 | 6.476 | 60.071 | −15.323 | 1.00 | 137.73 |
| 6613 | CB | GLU | E | 65 | 6.875 | 58.635 | −14.971 | 1.00 | 170.42 |
| 6614 | CG | GLU | E | 65 | 7.492 | 58.437 | −13.609 | 1.00 | 170.42 |
| 6615 | CD | GLU | E | 65 | 8.153 | 57.072 | −13.484 | 1.00 | 170.42 |
| 6616 | OE1 | GLU | E | 65 | 9.121 | 56.807 | −14.233 | 1.00 | 170.42 |
| 6617 | OE2 | GLU | E | 65 | 7.706 | 56.263 | −12.644 | 1.00 | 170.42 |
| 6618 | C | GLU | E | 65 | 7.645 | 61.025 | −15.096 | 1.00 | 137.73 |
| 6619 | O | GLU | E | 65 | 8.653 | 60.925 | −15.789 | 1.00 | 137.73 |
| 6620 | N | TYR | E | 66 | 7.513 | 61.952 | −14.147 | 1.00 | 117.13 |
| 6621 | CA | TYR | E | 66 | 8.588 | 62.908 | −13.864 | 1.00 | 117.13 |
| 6622 | CB | TYR | E | 66 | 8.123 | 64.321 | −14.112 | 1.00 | 93.74 |
| 6623 | CG | TYR | E | 66 | 7.767 | 64.647 | −15.528 | 1.00 | 93.74 |
| 6624 | CD1 | TYR | E | 66 | 6.586 | 64.214 | −16.090 | 1.00 | 93.74 |
| 6625 | CE1 | TYR | E | 66 | 6.220 | 64.609 | −17.382 | 1.00 | 93.74 |
| 6626 | CD2 | TYR | E | 66 | 8.582 | 65.471 | −16.285 | 1.00 | 93.74 |
| 6627 | CE2 | TYR | E | 66 | 8.230 | 65.873 | −17.564 | 1.00 | 93.74 |
| 6628 | CZ | TYR | E | 66 | 7.050 | 65.445 | −18.110 | 1.00 | 93.74 |
| 6629 | OH | TYR | E | 66 | 6.702 | 65.872 | −19.376 | 1.00 | 93.74 |
| 6630 | C | TYR | E | 66 | 9.062 | 62.852 | −12.426 | 1.00 | 117.13 |
| 6631 | O | TYR | E | 66 | 8.359 | 62.335 | −11.564 | 1.00 | 117.13 |
| 6632 | N | LYS | E | 67 | 10.248 | 63.402 | −12.169 | 1.00 | 125.36 |
| 6633 | CA | LYS | E | 67 | 10.802 | 63.443 | −10.815 | 1.00 | 125.36 |
| 6634 | CB | LYS | E | 67 | 11.186 | 62.037 | −10.352 | 1.00 | 181.51 |
| 6635 | CG | LYS | E | 67 | 12.026 | 61.282 | −11.345 | 1.00 | 181.51 |
| 6636 | CD | LYS | E | 67 | 12.264 | 59.876 | −10.876 | 1.00 | 181.51 |
| 6637 | CE | LYS | E | 67 | 12.938 | 59.066 | −11.956 | 1.00 | 181.51 |
| 6638 | NZ | LYS | E | 67 | 13.120 | 57.655 | −11.522 | 1.00 | 181.51 |
| 6639 | C | LYS | E | 67 | 12.014 | 64.362 | −10.732 | 1.00 | 125.36 |
| 6640 | O | LYS | E | 67 | 12.671 | 64.622 | −11.737 | 1.00 | 125.36 |
| 6641 | N | CYS | E | 68 | 12.289 | 64.881 | −9.541 | 1.00 | 114.74 |
| 6642 | CA | CYS | E | 68 | 13.451 | 65.729 | −9.370 | 1.00 | 114.74 |
| 6643 | C | CYS | E | 68 | 14.297 | 65.211 | −8.210 | 1.00 | 114.74 |
| 6644 | O | CYS | E | 68 | 13.824 | 64.428 | −7.388 | 1.00 | 114.74 |
| 6645 | CB | CYS | E | 68 | 13.047 | 67.197 | −9.159 | 1.00 | 167.12 |
| 6646 | SG | CYS | E | 68 | 12.001 | 67.607 | −7.729 | 1.00 | 167.12 |
| 6647 | N | GLN | E | 69 | 15.561 | 65.619 | −8.180 | 1.00 | 152.39 |
| 6648 | CA | GLN | E | 69 | 16.493 | 65.217 | −7.139 | 1.00 | 152.39 |
| 6649 | CB | GLN | E | 69 | 17.120 | 63.861 | −7.482 | 1.00 | 180.76 |
| 6650 | CG | GLN | E | 69 | 18.398 | 63.553 | −6.725 | 1.00 | 180.76 |
| 6651 | CD | GLN | E | 69 | 19.065 | 62.274 | −7.191 | 1.00 | 180.76 |
| 6652 | OE1 | GLN | E | 69 | 19.315 | 62.089 | −8.383 | 1.00 | 180.76 |
| 6653 | NE2 | GLN | E | 69 | 19.364 | 61.386 | −6.250 | 1.00 | 180.76 |
| 6654 | C | GLN | E | 69 | 17.566 | 66.292 | −7.067 | 1.00 | 152.39 |
| 6655 | O | GLN | E | 69 | 17.822 | 66.998 | −8.048 | 1.00 | 152.39 |
| 6656 | N | HIS | E | 70 | 18.186 | 66.429 | −5.902 | 1.00 | 249.25 |
| 6657 | CA | HIS | E | 70 | 19.226 | 67.429 | −5.730 | 1.00 | 249.25 |
| 6658 | CB | HIS | E | 70 | 18.911 | 68.308 | −4.519 | 1.00 | 185.63 |
| 6659 | CG | HIS | E | 70 | 17.717 | 69.187 | −4.713 | 1.00 | 185.63 |
| 6660 | CD2 | HIS | E | 70 | 16.426 | 69.026 | −4.338 | 1.00 | 185.63 |
| 6661 | ND1 | HIS | E | 70 | 17.769 | 70.377 | −5.406 | 1.00 | 185.63 |
| 6662 | CE1 | HIS | E | 70 | 16.560 | 70.915 | −5.449 | 1.00 | 185.63 |
| 6663 | NE2 | HIS | E | 70 | 15.729 | 70.114 | −4.806 | 1.00 | 185.63 |
| 6664 | C | HIS | E | 70 | 20.605 | 66.806 | −5.583 | 1.00 | 249.25 |
| 6665 | O | HIS | E | 70 | 20.793 | 65.603 | −5.787 | 1.00 | 249.25 |
| 6666 | N | GLN | E | 71 | 21.568 | 67.644 | −5.225 | 1.00 | 214.79 |
| 6667 | CA | GLN | E | 71 | 22.945 | 67.209 | −5.061 | 1.00 | 214.79 |
| 6668 | CB | GLN | E | 71 | 23.787 | 68.387 | −4.553 | 1.00 | 249.44 |
| 6669 | CG | GLN | E | 71 | 25.227 | 68.347 | −5.024 | 1.00 | 249.44 |
| 6670 | CD | GLN | E | 71 | 25.359 | 68.172 | −6.526 | 1.00 | 249.44 |
| 6671 | OE1 | GLN | E | 71 | 25.049 | 69.077 | −7.304 | 1.00 | 249.44 |
| 6672 | NE2 | GLN | E | 71 | 25.815 | 67.001 | −6.940 | 1.00 | 249.44 |
| 6673 | C | GLN | E | 71 | 23.055 | 66.005 | −4.119 | 1.00 | 214.79 |
| 6674 | O | GLN | E | 71 | 23.602 | 64.967 | −4.496 | 1.00 | 214.79 |
| 6675 | N | GLN | E | 72 | 22.517 | 66.140 | −2.906 | 1.00 | 224.12 |
| 6676 | CA | GLN | E | 72 | 22.569 | 65.060 | −1.906 | 1.00 | 224.12 |
| 6677 | CB | GLN | E | 72 | 23.396 | 65.488 | −0.694 | 1.00 | 220.92 |
| 6678 | CG | GLN | E | 72 | 23.660 | 64.401 | 0.347 | 1.00 | 220.92 |
| 6679 | CD | GLN | E | 72 | 24.599 | 64.863 | 1.460 | 1.00 | 220.92 |
| 6680 | OE1 | GLN | E | 72 | 25.746 | 65.260 | 1.224 | 1.00 | 220.92 |
| 6681 | NE2 | GLN | E | 72 | 24.108 | 64.811 | 2.684 | 1.00 | 220.92 |
| 6682 | C | GLN | E | 72 | 21.190 | 64.677 | −1.396 | 1.00 | 224.12 |
| 6683 | O | GLN | E | 72 | 20.938 | 64.719 | −0.192 | 1.00 | 224.12 |
| 6684 | N | VAL | E | 73 | 20.301 | 64.293 | −2.298 | 1.00 | 249.50 |
| 6685 | CA | VAL | E | 73 | 18.953 | 63.928 | −1.899 | 1.00 | 249.50 |
| 6686 | CB | VAL | E | 73 | 18.006 | 65.126 | −2.032 | 1.00 | 213.59 |
| 6687 | CG1 | VAL | E | 73 | 16.699 | 64.850 | −1.308 | 1.00 | 213.59 |
| 6688 | CG2 | VAL | E | 73 | 18.676 | 66.352 | −1.536 | 1.00 | 213.59 |
| 6689 | C | VAL | E | 73 | 18.410 | 62.814 | −2.776 | 1.00 | 249.50 |
| 6690 | O | VAL | E | 73 | 18.724 | 62.740 | −3.964 | 1.00 | 249.50 |
| 6691 | N | ASN | E | 74 | 17.584 | 61.952 | −2.192 | 1.00 | 249.52 |
| 6692 | CA | ASN | E | 74 | 16.994 | 60.858 | −2.946 | 1.00 | 249.52 |
| 6693 | CB | ASN | E | 74 | 16.515 | 59.760 | −1.994 | 1.00 | 169.17 |
| 6694 | CG | ASN | E | 74 | 17.620 | 59.273 | −1.081 | 1.00 | 169.17 |
| 6695 | OD1 | ASN | E | 74 | 18.757 | 59.080 | −1.522 | 1.00 | 169.17 |
| 6696 | ND2 | ASN | E | 74 | 17.291 | 59.065 | 0.192 | 1.00 | 169.17 |
| 6697 | C | ASN | E | 74 | 15.838 | 61.389 | −3.787 | 1.00 | 249.52 |
| 6698 | O | ASN | E | 74 | 14.956 | 62.085 | −3.283 | 1.00 | 249.52 |
| 6699 | N | GLU | E | 75 | 15.870 | 61.071 | −5.077 | 1.00 | 220.79 |
| 6700 | CA | GLU | E | 75 | 14.851 | 61.501 | −6.025 | 1.00 | 220.79 |
| 6701 | CB | GLU | E | 75 | 14.992 | 60.694 | −7.316 | 1.00 | 206.72 |
| 6702 | CG | GLU | E | 75 | 15.456 | 59.259 | −7.100 | 1.00 | 206.72 |
| 6703 | CD | GLU | E | 75 | 15.805 | 58.557 | −8.399 | 1.00 | 206.72 |
| 6704 | OE1 | GLU | E | 75 | 16.636 | 59.096 | −9.159 | 1.00 | 206.72 |
| 6705 | OE2 | GLU | E | 75 | 15.254 | 57.465 | −8.660 | 1.00 | 206.72 |
| 6706 | C | GLU | E | 75 | 13.420 | 61.414 | −5.488 | 1.00 | 220.79 |
| 6707 | O | GLU | E | 75 | 13.071 | 60.495 | −4.747 | 1.00 | 220.79 |
| 6708 | N | SER | E | 76 | 12.604 | 62.385 | −5.885 | 1.00 | 123.65 |
| 6709 | CA | SER | E | 76 | 11.211 | 62.502 | −5.457 | 1.00 | 123.65 |
| 6710 | CB | SER | E | 76 | 10.646 | 63.837 | −5.918 | 1.00 | 156.05 |
| 6711 | OG | SER | E | 76 | 10.583 | 63.854 | −7.332 | 1.00 | 156.05 |
| 6712 | C | SER | E | 76 | 10.310 | 61.422 | −5.997 | 1.00 | 123.65 |
| 6713 | O | SER | E | 76 | 10.623 | 60.793 | −7.006 | 1.00 | 123.65 |
| 6714 | N | GLU | E | 77 | 9.172 | 61.235 | −5.333 | 1.00 | 207.91 |
| 6715 | CA | GLU | E | 77 | 8.193 | 60.247 | −5.762 | 1.00 | 207.91 |
| 6716 | CB | GLU | E | 77 | 7.055 | 60.135 | −4.744 | 1.00 | 181.88 |
| 6717 | CG | GLU | E | 77 | 7.495 | 59.613 | −3.388 | 1.00 | 181.88 |
| 6718 | CD | GLU | E | 77 | 8.000 | 58.181 | −3.440 | 1.00 | 181.88 |
| 6719 | OE1 | GLU | E | 77 | 8.281 | 57.684 | −4.551 | 1.00 | 181.88 |
| 6720 | OE2 | GLU | E | 77 | 8.127 | 57.550 | −2.369 | 1.00 | 181.88 |
| 6721 | C | GLU | E | 77 | 7.645 | 60.711 | −7.103 | 1.00 | 207.91 |
| 6722 | O | GLU | E | 77 | 7.050 | 61.788 | −7.189 | 1.00 | 207.91 |
| 6723 | N | PRO | E | 78 | 7.857 | 59.919 | −8.171 | 1.00 | 80.79 |
| 6724 | CD | PRO | E | 78 | 8.716 | 58.718 | −8.203 | 1.00 | 240.65 |
| 6725 | CA | PRO | E | 78 | 7.390 | 60.237 | −9.522 | 1.00 | 80.79 |
| 6726 | CB | PRO | E | 78 | 7.588 | 58.930 | −10.260 | 1.00 | 240.65 |
| 6727 | CG | PRO | E | 78 | 8.884 | 58.468 | −9.699 | 1.00 | 240.65 |
| 6728 | C | PRO | E | 78 | 5.967 | 60.738 | −9.594 | 1.00 | 80.79 |
| 6729 | O | PRO | E | 78 | 5.145 | 60.436 | −8.738 | 1.00 | 80.79 |
| 6730 | N | VAL | E | 79 | 5.681 | 61.517 | −10.622 | 1.00 | 112.90 |
| 6731 | CA | VAL | E | 79 | 4.351 | 62.067 | −10.801 | 1.00 | 112.90 |
| 6732 | CB | VAL | E | 79 | 4.314 | 63.550 | −10.455 | 1.00 | 137.47 |
| 6733 | CG1 | VAL | E | 79 | 3.032 | 64.176 | −10.987 | 1.00 | 137.47 |
| 6734 | CG2 | VAL | E | 79 | 4.403 | 63.715 | −8.951 | 1.00 | 137.47 |
| 6735 | C | VAL | E | 79 | 3.978 | 61.908 | −12.251 | 1.00 | 112.90 |
| 6736 | O | VAL | E | 79 | 4.737 | 62.312 | −13.132 | 1.00 | 112.90 |
| 6737 | N | TYR | E | 80 | 2.807 | 61.334 | −12.508 | 1.00 | 70.76 |
| 6738 | CA | TYR | E | 80 | 2.402 | 61.134 | −13.886 | 1.00 | 70.76 |
| 6739 | CB | TYR | E | 80 | 1.630 | 59.841 | −14.062 | 1.00 | 159.99 |
| 6740 | CG | TYR | E | 80 | 1.595 | 59.441 | −15.510 | 1.00 | 159.99 |
| 6741 | CD1 | TYR | E | 80 | 2.763 | 59.078 | −16.169 | 1.00 | 159.99 |
| 6742 | CE1 | TYR | E | 80 | 2.758 | 58.714 | −17.498 | 1.00 | 159.99 |
| 6743 | CD2 | TYR | E | 80 | 0.408 | 59.437 | −16.231 | 1.00 | 159.99 |
| 6744 | CE2 | TYR | E | 80 | 0.395 | 59.076 | −17.589 | 1.00 | 159.99 |
| 6745 | CZ | TYR | E | 80 | 1.583 | 58.712 | −18.206 | 1.00 | 159.99 |
| 6746 | OH | TYR | E | 80 | 1.597 | 58.323 | −19.528 | 1.00 | 159.99 |
| 6747 | C | TYR | E | 80 | 1.562 | 62.238 | −14.446 | 1.00 | 70.76 |
| 6748 | O | TYR | E | 80 | 0.661 | 62.746 | −13.795 | 1.00 | 70.76 |
| 6749 | N | LEU | E | 81 | 1.838 | 62.587 | −15.684 | 1.00 | 117.40 |
| 6750 | CA | LEU | E | 81 | 1.086 | 63.620 | −16.343 | 1.00 | 117.40 |
| 6751 | CB | LEU | E | 81 | 2.037 | 64.731 | −16.752 | 1.00 | 104.60 |
| 6752 | CG | LEU | E | 81 | 1.348 | 65.795 | −17.590 | 1.00 | 104.60 |
| 6753 | CD1 | LEU | E | 81 | 0.269 | 66.455 | −16.750 | 1.00 | 104.60 |
| 6754 | CD2 | LEU | E | 81 | 2.357 | 66.811 | −18.058 | 1.00 | 104.60 |
| 6755 | C | LEU | E | 81 | 0.455 | 62.990 | −17.575 | 1.00 | 117.40 |
| 6756 | O | LEU | E | 81 | 1.148 | 62.313 | −18.325 | 1.00 | 117.40 |
| 6757 | N | GLU | E | 82 | −0.844 | 63.186 | −17.792 | 1.00 | 85.04 |
| 6758 | CA | GLU | E | 82 | −1.474 | 62.609 | −18.982 | 1.00 | 85.04 |
| 6759 | CB | GLU | E | 82 | −2.520 | 61.566 | −18.598 | 1.00 | 152.39 |
| 6760 | CG | GLU | E | 82 | −2.736 | 60.525 | −19.684 | 1.00 | 152.39 |
| 6761 | CD | GLU | E | 82 | −3.765 | 59.482 | −19.297 | 1.00 | 152.39 |
| 6762 | OE1 | GLU | E | 82 | −3.785 | 59.083 | −18.107 | 1.00 | 152.39 |
| 6763 | OE2 | GLU | E | 82 | −4.542 | 59.055 | −20.185 | 1.00 | 152.39 |
| 6764 | C | GLU | E | 82 | −2.112 | 63.691 | −19.854 | 1.00 | 85.04 |
| 6765 | O | GLU | E | 82 | −2.843 | 64.562 | −19.363 | 1.00 | 85.04 |
| 6766 | N | VAL | E | 83 | −1.829 | 63.634 | −21.152 | 1.00 | 88.74 |
| 6767 | CA | VAL | E | 83 | −2.355 | 64.628 | −22.085 | 1.00 | 88.74 |
| 6768 | CB | VAL | E | 83 | −1.258 | 65.146 | −23.010 | 1.00 | 170.11 |
| 6769 | CG1 | VAL | E | 83 | −1.862 | 66.064 | −24.054 | 1.00 | 170.11 |
| 6770 | CG2 | VAL | E | 83 | −0.213 | 65.878 | −22.197 | 1.00 | 170.11 |
| 6771 | C | VAL | E | 83 | −3.482 | 64.102 | −22.949 | 1.00 | 88.74 |
| 6772 | O | VAL | E | 83 | −3.391 | 63.013 | −23.507 | 1.00 | 88.74 |
| 6773 | N | PHE | E | 84 | −4.534 | 64.895 | −23.091 | 1.00 | 98.95 |
| 6774 | CA | PHE | E | 84 | −5.675 | 64.451 | −23.873 | 1.00 | 98.95 |
| 6775 | CB | PHE | E | 84 | −6.917 | 64.273 | −22.986 | 1.00 | 118.45 |
| 6776 | CG | PHE | E | 84 | −6.734 | 63.316 | −21.864 | 1.00 | 118.45 |
| 6777 | CD1 | PHE | E | 84 | −5.997 | 63.674 | −20.749 | 1.00 | 118.45 |
| 6778 | CD2 | PHE | E | 84 | −7.324 | 62.065 | −21.911 | 1.00 | 118.45 |
| 6779 | CE1 | PHE | E | 84 | −5.839 | 62.795 | −19.701 | 1.00 | 118.45 |
| 6780 | CE2 | PHE | E | 84 | −7.172 | 61.173 | −20.865 | 1.00 | 118.45 |
| 6781 | CZ | PHE | E | 84 | −6.434 | 61.541 | −19.756 | 1.00 | 118.45 |
| 6782 | C | PHE | E | 84 | −6.114 | 65.318 | −25.034 | 1.00 | 98.95 |
| 6783 | O | PHE | E | 84 | −5.736 | 66.484 | −25.178 | 1.00 | 98.95 |
| 6784 | N | SER | E | 85 | −6.967 | 64.697 | −25.837 | 1.00 | 152.83 |
| 6785 | CA | SER | E | 85 | −7.592 | 65.304 | −26.990 | 1.00 | 152.83 |
| 6786 | CB | SER | E | 85 | −6.937 | 64.819 | −28.279 | 1.00 | 197.25 |
| 6787 | OG | SER | E | 85 | −7.565 | 65.403 | −29.405 | 1.00 | 197.25 |
| 6788 | C | SER | E | 85 | −9.030 | 64.791 | −26.914 | 1.00 | 152.83 |
| 6789 | O | SER | E | 85 | −9.279 | 63.598 | −27.101 | 1.00 | 152.83 |
| 6790 | N | ASP | E | 86 | −9.962 | 65.685 | −26.600 | 1.00 | 101.99 |
| 6791 | CA | ASP | E | 86 | −11.375 | 65.330 | −26.494 | 1.00 | 101.99 |
| 6792 | CB | ASP | E | 86 | −11.573 | 64.296 | −25.386 | 1.00 | 136.02 |
| 6793 | CG | ASP | E | 86 | −12.655 | 63.310 | −25.715 | 1.00 | 136.02 |
| 6794 | OD1 | ASP | E | 86 | −13.782 | 63.752 | −26.031 | 1.00 | 136.02 |
| 6795 | OD2 | ASP | E | 86 | −12.375 | 62.095 | −25.664 | 1.00 | 136.02 |
| 6796 | C | ASP | E | 86 | −12.199 | 66.594 | −26.197 | 1.00 | 101.99 |
| 6797 | O | ASP | E | 86 | −11.646 | 67.635 | −25.830 | 1.00 | 101.99 |
| 6798 | N | TRP | E | 87 | −13.516 | 66.516 | −26.356 | 1.00 | 94.76 |
| 6799 | CA | TRP | E | 87 | −14.351 | 67.689 | −26.106 | 1.00 | 94.76 |
| 6800 | CB | TRP | E | 87 | −15.806 | 67.417 | −26.477 | 1.00 | 229.11 |
| 6801 | CG | TRP | E | 87 | −16.051 | 67.695 | −27.896 | 1.00 | 229.11 |
| 6802 | CD2 | TRP | E | 87 | −15.956 | 66.755 | −28.964 | 1.00 | 229.11 |
| 6803 | CE2 | TRP | E | 87 | −16.120 | 67.470 | −30.165 | 1.00 | 229.11 |
| 6804 | CE3 | TRP | E | 87 | −15.735 | 65.372 | −29.026 | 1.00 | 229.11 |
| 6805 | CD1 | TRP | E | 87 | −16.281 | 68.917 | −28.471 | 1.00 | 229.11 |
| 6806 | NE1 | TRP | E | 87 | −16.319 | 68.787 | −29.837 | 1.00 | 229.11 |
| 6807 | CZ2 | TRP | E | 87 | −16.070 | 66.850 | −31.411 | 1.00 | 229.11 |
| 6808 | CZ3 | TRP | E | 87 | −15.685 | 64.755 | −30.265 | 1.00 | 229.11 |
| 6809 | CH2 | TRP | E | 87 | −15.855 | 65.491 | −31.438 | 1.00 | 229.11 |
| 6810 | C | TRP | E | 87 | −14.273 | 68.108 | −24.671 | 1.00 | 94.76 |
| 6811 | O | TRP | E | 87 | −13.962 | 69.260 | −24.355 | 1.00 | 94.76 |
| 6812 | N | LEU | E | 88 | −14.546 | 67.147 | −23.802 | 1.00 | 160.64 |
| 6813 | CA | LEU | E | 88 | −14.527 | 67.385 | −22.379 | 1.00 | 160.64 |
| 6814 | CB | LEU | E | 88 | −15.912 | 67.160 | −21.803 | 1.00 | 93.36 |
| 6815 | CG | LEU | E | 88 | −16.950 | 68.149 | −22.276 | 1.00 | 93.36 |
| 6816 | CD1 | LEU | E | 88 | −18.247 | 67.872 | −21.532 | 1.00 | 93.36 |
| 6817 | CD2 | LEU | E | 88 | −16.456 | 69.560 | −22.001 | 1.00 | 93.36 |
| 6818 | C | LEU | E | 88 | −13.553 | 66.490 | −21.645 | 1.00 | 160.64 |
| 6819 | O | LEU | E | 88 | −13.401 | 65.314 | −21.968 | 1.00 | 160.64 |
| 6820 | N | LEU | E | 89 | −12.908 | 67.057 | −20.635 | 1.00 | 107.53 |
| 6821 | CA | LEU | E | 89 | −11.961 | 66.321 | −19.828 | 1.00 | 107.53 |
| 6822 | CB | LEU | E | 89 | −10.552 | 66.785 | −20.131 | 1.00 | 83.31 |
| 6823 | CG | LEU | E | 89 | −9.538 | 66.057 | −19.267 | 1.00 | 83.31 |
| 6824 | CD1 | LEU | E | 89 | −9.821 | 64.541 | −19.311 | 1.00 | 83.31 |
| 6825 | CD2 | LEU | E | 89 | −8.138 | 66.385 | −19.757 | 1.00 | 83.31 |
| 6826 | C | LEU | E | 89 | −12.252 | 66.573 | −18.366 | 1.00 | 107.53 |
| 6827 | O | LEU | E | 89 | −12.378 | 67.718 | −17.954 | 1.00 | 107.53 |
| 6828 | N | LEU | E | 90 | −12.366 | 65.510 | −17.576 | 1.00 | 62.89 |
| 6829 | CA | LEU | E | 90 | −12.629 | 65.676 | −16.142 | 1.00 | 62.89 |
| 6830 | CB | LEU | E | 90 | −13.400 | 64.487 | −15.588 | 1.00 | 49.26 |
| 6831 | CG | LEU | E | 90 | −13.609 | 64.523 | −14.077 | 1.00 | 49.26 |
| 6832 | CD1 | LEU | E | 90 | −14.422 | 65.760 | −13.775 | 1.00 | 49.26 |
| 6833 | CD2 | LEU | E | 90 | −14.308 | 63.261 | −13.579 | 1.00 | 49.26 |
| 6834 | C | LEU | E | 90 | −11.300 | 65.773 | −15.404 | 1.00 | 62.89 |
| 6835 | O | LEU | E | 90 | −10.515 | 64.830 | −15.410 | 1.00 | 62.89 |
| 6836 | N | GLN | E | 91 | −11.043 | 66.907 | −14.764 | 1.00 | 69.52 |
| 6837 | CA | GLN | E | 91 | −9.785 | 67.083 | −14.064 | 1.00 | 69.52 |
| 6838 | CB | GLN | E | 91 | −9.210 | 68.449 | −14.388 | 1.00 | 103.30 |
| 6839 | CG | GLN | E | 91 | −8.977 | 68.644 | −15.857 | 1.00 | 103.30 |
| 6840 | CD | GLN | E | 91 | −8.226 | 69.921 | −16.149 | 1.00 | 103.30 |
| 6841 | OE1 | GLN | E | 91 | −8.750 | 71.021 | −15.967 | 1.00 | 103.30 |
| 6842 | NE2 | GLN | E | 91 | −6.979 | 69.783 | −16.599 | 1.00 | 103.30 |
| 6843 | C | GLN | E | 91 | −9.965 | 66.953 | −12.584 | 1.00 | 69.52 |
| 6844 | O | GLN | E | 91 | −10.984 | 67.372 | −12.033 | 1.00 | 69.52 |
| 6845 | N | ALA | E | 92 | −8.972 | 66.375 | −11.925 | 1.00 | 61.94 |
| 6846 | CA | ALA | E | 92 | −9.070 | 66.223 | −10.483 | 1.00 | 61.94 |
| 6847 | CB | ALA | E | 92 | −9.241 | 64.773 | −10.125 | 1.00 | 129.56 |
| 6848 | C | ALA | E | 92 | −7.838 | 66.792 | −9.794 | 1.00 | 61.94 |
| 6849 | O | ALA | E | 92 | −6.715 | 66.663 | −10.314 | 1.00 | 61.94 |
| 6850 | N | SER | E | 93 | −8.045 | 67.437 | −8.643 | 1.00 | 82.97 |
| 6851 | CA | SER | E | 93 | −6.930 | 68.004 | −7.904 | 1.00 | 82.97 |
| 6852 | CB | SER | E | 93 | −7.388 | 68.550 | −6.552 | 1.00 | 72.43 |
| 6853 | OG | SER | E | 93 | −8.203 | 67.620 | −5.871 | 1.00 | 72.43 |
| 6854 | C | SER | E | 93 | −5.965 | 66.855 | −7.717 | 1.00 | 82.97 |
| 6855 | O | SER | E | 93 | −4.873 | 66.846 | −8.282 | 1.00 | 82.97 |
| 6856 | N | ALA | E | 94 | −6.395 | 65.865 | −6.948 | 1.00 | 109.69 |
| 6857 | CA | ALA | E | 94 | −5.588 | 64.680 | −6.683 | 1.00 | 109.69 |
| 6858 | CB | ALA | E | 94 | −5.086 | 64.711 | −5.262 | 1.00 | 145.34 |
| 6859 | C | ALA | E | 94 | −6.468 | 63.455 | −6.910 | 1.00 | 109.69 |
| 6860 | O | ALA | E | 94 | −7.652 | 63.488 | −6.620 | 1.00 | 109.69 |
| 6861 | N | GLU | E | 95 | −5.902 | 62.372 | −7.431 | 1.00 | 77.50 |
| 6862 | CA | GLU | E | 95 | −6.688 | 61.172 | −7.711 | 1.00 | 77.50 |
| 6863 | CB | GLU | E | 95 | −6.065 | 60.391 | −8.859 | 1.00 | 137.79 |
| 6864 | CG | GLU | E | 95 | −5.979 | 61.195 | −10.136 | 1.00 | 137.79 |
| 6865 | CD | GLU | E | 95 | −5.700 | 60.335 | −11.358 | 1.00 | 137.79 |
| 6866 | OE1 | GLU | E | 95 | −5.582 | 60.901 | −12.468 | 1.00 | 137.79 |
| 6867 | OE2 | GLU | E | 95 | −5.607 | 59.094 | −11.210 | 1.00 | 137.79 |
| 6868 | C | GLU | E | 95 | −6.869 | 60.258 | −6.514 | 1.00 | 77.50 |
| 6869 | O | GLU | E | 95 | −7.723 | 59.376 | −6.538 | 1.00 | 77.50 |
| 6870 | N | VAL | E | 96 | −6.067 | 60.468 | −5.470 | 1.00 | 83.99 |
| 6871 | CA | VAL | E | 96 | −6.148 | 59.673 | −4.241 | 1.00 | 83.99 |
| 6872 | CB | VAL | E | 96 | −5.042 | 58.641 | −4.191 | 1.00 | 76.64 |
| 6873 | CG1 | VAL | E | 96 | −5.384 | 57.579 | −3.164 | 1.00 | 76.64 |
| 6874 | CG2 | VAL | E | 96 | −4.856 | 58.037 | −5.568 | 1.00 | 76.64 |
| 6875 | C | VAL | E | 96 | −6.009 | 60.634 | −3.071 | 1.00 | 83.99 |
| 6876 | O | VAL | E | 96 | −5.127 | 61.491 | −3.057 | 1.00 | 83.99 |
| 6877 | N | VAL | E | 97 | −6.863 | 60.475 | −2.071 | 1.00 | 86.29 |
| 6878 | CA | VAL | E | 97 | −6.880 | 61.409 | −0.959 | 1.00 | 86.29 |
| 6879 | CB | VAL | E | 97 | −8.028 | 62.389 | −1.152 | 1.00 | 60.14 |
| 6880 | CG1 | VAL | E | 97 | −7.861 | 63.550 | −0.242 | 1.00 | 80.14 |
| 6881 | CG2 | VAL | E | 97 | −8.102 | 62.821 | −2.571 | 1.00 | 80.14 |
| 6882 | C | VAL | E | 97 | −7.073 | 60.838 | 0.440 | 1.00 | 86.29 |
| 6883 | O | VAL | E | 97 | −7.940 | 59.988 | 0.646 | 1.00 | 86.29 |
| 6884 | N | MET | E | 98 | −6.305 | 61.349 | 1.405 | 1.00 | 72.65 |
| 6885 | CA | MET | E | 98 | −6.430 | 60.922 | 2.799 | 1.00 | 72.65 |
| 6886 | CB | MET | E | 98 | −5.268 | 61.476 | 3.603 | 1.00 | 162.98 |
| 6887 | CG | MET | E | 98 | −3.950 | 60.925 | 3.147 | 1.00 | 162.98 |
| 6888 | SD | MET | E | 98 | −3.643 | 59.309 | 3.816 | 1.00 | 162.98 |
| 6889 | CE | MET | E | 98 | −3.249 | 59.757 | 5.511 | 1.00 | 162.98 |
| 6890 | C | MET | E | 98 | −7.747 | 61.457 | 3.359 | 1.00 | 72.65 |
| 6891 | O | MET | E | 98 | −8.065 | 62.625 | 3.165 | 1.00 | 72.65 |
| 6892 | N | GLU | E | 99 | −8.515 | 60.612 | 4.042 | 1.00 | 91.21 |
| 6893 | CA | GLU | E | 99 | −9.789 | 61.038 | 4.615 | 1.00 | 91.21 |
| 6894 | CB | GLU | E | 99 | −10.288 | 60.011 | 5.630 | 1.00 | 221.21 |
| 6895 | CG | GLU | E | 99 | −11.780 | 60.081 | 5.888 | 1.00 | 221.21 |
| 6896 | CD | GLU | E | 99 | −12.193 | 59.288 | 7.112 | 1.00 | 221.21 |
| 6897 | OE1 | GLU | E | 99 | −11.617 | 58.201 | 7.342 | 1.00 | 221.21 |
| 6898 | OE2 | GLU | E | 99 | −13.102 | 59.747 | 7.836 | 1.00 | 221.21 |
| 6899 | C | GLU | E | 99 | −9.575 | 62.376 | 5.322 | 1.00 | 91.21 |
| 6900 | O | GLU | E | 99 | −8.664 | 62.499 | 6.140 | 1.00 | 91.21 |
| 6901 | N | GLY | E | 100 | −10.388 | 63.379 | 4.993 | 1.00 | 149.52 |
| 6902 | CA | GLY | E | 100 | −10.248 | 64.679 | 5.632 | 1.00 | 149.52 |
| 6903 | C | GLY | E | 100 | −9.666 | 65.799 | 4.782 | 1.00 | 149.52 |
| 6904 | O | GLY | E | 100 | −9.830 | 66.974 | 5.111 | 1.00 | 149.52 |
| 6905 | N | GLN | E | 101 | −8.982 | 65.450 | 3.697 | 1.00 | 88.18 |
| 6906 | CA | GLN | E | 101 | −8.381 | 66.447 | 2.803 | 1.00 | 88.18 |
| 6907 | CB | GLN | E | 101 | −7.183 | 65.843 | 2.072 | 1.00 | 168.37 |
| 6908 | CG | GLN | E | 101 | −6.053 | 65.456 | 2.988 | 1.00 | 168.37 |
| 6909 | CD | GLN | E | 101 | −5.680 | 66.573 | 3.934 | 1.00 | 168.37 |
| 6910 | OE1 | GLN | E | 101 | −6.368 | 66.812 | 4.923 | 1.00 | 168.37 |
| 6911 | NE2 | GLN | E | 101 | −4.598 | 67.272 | 3.630 | 1.00 | 168.37 |
| 6912 | C | GLN | E | 101 | −9.334 | 67.070 | 1.767 | 1.00 | 88.18 |
| 6913 | O | GLN | E | 101 | −10.447 | 66.597 | 1.540 | 1.00 | 88.18 |
| 6914 | N | PRO | E | 102 | −8.894 | 68.148 | 1.116 | 1.00 | 90.47 |
| 6915 | CD | PRO | E | 102 | −7.628 | 68.898 | 1.252 | 1.00 | 130.24 |
| 6916 | CA | PRO | E | 102 | −9.766 | 68.772 | 0.125 | 1.00 | 90.47 |
| 6917 | CB | PRO | E | 102 | −9.195 | 70.179 | 0.022 | 1.00 | 130.24 |
| 6918 | CG | PRO | E | 102 | −7.727 | 69.923 | 0.124 | 1.00 | 130.24 |
| 6919 | C | PRO | E | 102 | −9.708 | 68.020 | −1.203 | 1.00 | 90.47 |
| 6920 | O | PRO | E | 102 | −8.713 | 67.360 | −1.510 | 1.00 | 90.47 |
| 6921 | N | LEU | E | 103 | −10.775 | 68.133 | −1.988 | 1.00 | 92.93 |
| 6922 | CA | LEU | E | 103 | −10.853 | 67.475 | −3.283 | 1.00 | 92.93 |
| 6923 | CB | LEU | E | 103 | −11.638 | 66.186 | −3.160 | 1.00 | 78.05 |
| 6924 | CG | LEU | E | 103 | −11.718 | 65.529 | −4.528 | 1.00 | 78.05 |
| 6925 | CD1 | LEU | E | 103 | −10.330 | 65.172 | −4.969 | 1.00 | 78.05 |
| 6926 | CD2 | LEU | E | 103 | −12.618 | 64.299 | −4.466 | 1.00 | 78.05 |
| 6927 | C | LEU | E | 103 | −11.556 | 68.364 | −4.296 | 1.00 | 92.93 |
| 6928 | O | LEU | E | 103 | −12.690 | 68.784 | −4.071 | 1.00 | 92.93 |
| 6929 | N | PHE | E | 104 | −10.906 | 68.654 | −5.413 | 1.00 | 62.11 |
| 6930 | CA | PHE | E | 104 | −11.557 | 69.491 | −6.406 | 1.00 | 62.11 |
| 6931 | CB | PHE | E | 104 | −10.792 | 70.814 | −6.607 | 1.00 | 152.14 |
| 6932 | CG | PHE | E | 104 | −10.639 | 71.636 | −5.354 | 1.00 | 152.14 |
| 6933 | CD1 | PHE | E | 104 | −9.688 | 71.298 | −4.401 | 1.00 | 152.14 |
| 6934 | CD2 | PHE | E | 104 | −11.439 | 72.760 | −5.132 | 1.00 | 152.14 |
| 6935 | CE1 | PHE | E | 104 | −9.537 | 72.054 | −3.235 | 1.00 | 152.14 |
| 6936 | CE2 | PHE | E | 104 | −11.297 | 73.526 | −3.967 | 1.00 | 152.14 |
| 6937 | CZ | PHE | E | 104 | −10.338 | 73.173 | −3.020 | 1.00 | 152.14 |
| 6938 | C | PHE | E | 104 | −11.672 | 68.775 | −7.759 | 1.00 | 62.11 |
| 6939 | O | PHE | E | 104 | −10.686 | 68.246 | −8.269 | 1.00 | 62.11 |
| 6940 | N | LEU | E | 105 | −12.871 | 68.748 | −8.336 | 1.00 | 83.91 |
| 6941 | CA | LEU | E | 105 | −13.074 | 68.140 | −9.651 | 1.00 | 83.91 |
| 6942 | CB | LEU | E | 105 | −14.208 | 67.119 | −9.618 | 1.00 | 47.95 |
| 6943 | CG | LEU | E | 105 | −14.010 | 66.057 | −8.551 | 1.00 | 47.95 |
| 6944 | CD1 | LEU | E | 105 | −15.076 | 64.958 | −8.677 | 1.00 | 47.95 |
| 6945 | CD2 | LEU | E | 105 | −12.633 | 65.508 | −8.729 | 1.00 | 47.95 |
| 6946 | C | LEU | E | 105 | −13.454 | 69.279 | −10.565 | 1.00 | 83.91 |
| 6947 | O | LEU | E | 105 | −14.110 | 70.224 | −10.133 | 1.00 | 83.91 |
| 6948 | N | ARG | E | 106 | −13.063 | 69.201 | −11.824 | 1.00 | 105.55 |
| 6949 | CA | ARG | E | 106 | −13.391 | 70.273 | −12.737 | 1.00 | 105.55 |
| 6950 | CB | ARG | E | 106 | −12.182 | 71.181 | −12.874 | 1.00 | 149.26 |
| 6951 | CG | ARG | E | 106 | −12.358 | 72.290 | −13.865 | 1.00 | 149.26 |
| 6952 | CD | ARG | E | 106 | −11.036 | 72.987 | −14.126 | 1.00 | 149.26 |
| 6953 | NE | ARG | E | 106 | −11.165 | 74.010 | −15.158 | 1.00 | 149.26 |
| 6954 | CZ | ARG | E | 106 | −10.153 | 74.477 | −15.880 | 1.00 | 149.26 |
| 6955 | NH1 | ARG | E | 106 | −8.925 | 74.013 | −15.691 | 1.00 | 149.26 |
| 6956 | NH2 | ARG | E | 106 | −10.374 | 75.404 | −16.800 | 1.00 | 149.26 |
| 6957 | C | ARG | E | 106 | −13.804 | 69.761 | −14.110 | 1.00 | 105.55 |
| 6958 | O | ARG | E | 106 | −13.044 | 69.027 | −14.734 | 1.00 | 105.55 |
| 6959 | N | CYS | E | 107 | −15.000 | 70.118 | −14.585 | 1.00 | 115.02 |
| 6960 | CA | CYS | E | 107 | −15.400 | 69.665 | −15.913 | 1.00 | 115.02 |
| 6961 | C | CYS | E | 107 | −14.789 | 70.673 | −16.856 | 1.00 | 115.02 |
| 6962 | O | CYS | E | 107 | −15.230 | 71.813 | −16.932 | 1.00 | 115.02 |
| 6963 | CB | CYS | E | 107 | −16.914 | 69.630 | −16.079 | 1.00 | 134.10 |
| 6964 | SG | CYS | E | 107 | −17.441 | 68.605 | −17.498 | 1.00 | 134.10 |
| 6965 | N | HIS | E | 108 | −13.755 | 70.242 | −17.562 | 1.00 | 96.54 |
| 6966 | CA | HIS | E | 108 | −13.017 | 71.109 | −18.460 | 1.00 | 96.54 |
| 6967 | CB | HIS | E | 108 | −11.535 | 70.876 | −18.240 | 1.00 | 124.72 |
| 6968 | CG | HIS | E | 108 | −10.657 | 71.846 | −18.959 | 1.00 | 124.72 |
| 6969 | CD2 | HIS | E | 108 | −9.644 | 71.651 | −19.836 | 1.00 | 124.72 |
| 6970 | ND1 | HIS | E | 108 | −10.737 | 73.207 | −18.759 | 1.00 | 124.72 |
| 6971 | CE1 | HIS | E | 108 | −9.806 | 73.807 | −19.480 | 1.00 | 124.72 |
| 6972 | NE2 | HIS | E | 108 | −9.128 | 72.886 | −20.142 | 1.00 | 124.72 |
| 6973 | C | HIS | E | 108 | −13.328 | 70.954 | −19.938 | 1.00 | 96.54 |
| 6974 | O | HIS | E | 108 | −13.224 | 69.864 | −20.506 | 1.00 | 96.54 |
| 6975 | N | GLY | E | 109 | −13.680 | 72.070 | −20.563 | 1.00 | 176.91 |
| 6976 | CA | GLY | E | 109 | −13.999 | 72.050 | −21.973 | 1.00 | 176.91 |
| 6977 | C | GLY | E | 109 | −12.753 | 72.194 | −22.815 | 1.00 | 176.91 |
| 6978 | O | GLY | E | 109 | −11.737 | 72.706 | −22.349 | 1.00 | 176.91 |
| 6979 | N | TRP | E | 110 | −12.826 | 71.732 | −24.056 | 1.00 | 146.96 |
| 6980 | CA | TRP | E | 110 | −11.696 | 71.836 | −24.956 | 1.00 | 146.96 |
| 6981 | CB | TRP | E | 110 | −11.982 | 71.061 | −26.241 | 1.00 | 177.84 |
| 6982 | CG | TRP | E | 110 | −10.936 | 71.248 | −27.286 | 1.00 | 177.84 |
| 6983 | CD2 | TRP | E | 110 | −9.797 | 70.415 | −27.514 | 1.00 | 177.84 |
| 6984 | CE2 | TRP | E | 110 | −9.056 | 70.996 | −28.564 | 1.00 | 177.84 |
| 6985 | CE3 | TRP | E | 110 | −9.331 | 69.226 | −26.932 | 1.00 | 177.84 |
| 6986 | CD1 | TRP | E | 110 | −10.845 | 72.273 | −28.180 | 1.00 | 177.84 |
| 6987 | NE1 | TRP | E | 110 | −9.718 | 72.131 | −28.953 | 1.00 | 177.84 |
| 6988 | CZ2 | TRP | E | 110 | −7.875 | 70.433 | −29.047 | 1.00 | 177.84 |
| 6989 | CZ3 | TRP | E | 110 | −8.153 | 68.667 | −27.413 | 1.00 | 177.84 |
| 6990 | CH2 | TRP | E | 110 | −7.439 | 69.273 | −28.459 | 1.00 | 177.84 |
| 6991 | C | TRP | E | 110 | −11.404 | 73.303 | −25.275 | 1.00 | 146.96 |
| 6992 | O | TRP | E | 110 | −12.300 | 74.154 | −25.255 | 1.00 | 146.96 |
| 6993 | N | ARG | E | 111 | −10.136 | 73.594 | −25.551 | 1.00 | 199.97 |
| 6994 | CA | ARG | E | 111 | −9.716 | 74.948 | −25.887 | 1.00 | 199.97 |
| 6995 | CB | ARG | E | 111 | −10.136 | 75.282 | −27.295 | 1.00 | 249.42 |
| 6996 | CG | ARG | E | 111 | −9.116 | 74.871 | −28.285 | 1.00 | 249.42 |
| 6997 | CD | ARG | E | 111 | −9.462 | 75.454 | −29.595 | 1.00 | 249.42 |
| 6998 | NE | ARG | E | 111 | −8.270 | 75.956 | −30.249 | 1.00 | 249.42 |
| 6999 | CZ | ARG | E | 111 | −7.559 | 76.996 | −29.814 | 1.00 | 249.42 |
| 7000 | NH1 | ARG | E | 111 | −7.918 | 77.656 | −28.711 | 1.00 | 249.42 |
| 7001 | NH2 | ARG | E | 111 | −6.472 | 77.367 | −30.483 | 1.00 | 249.42 |
| 7002 | C | ARG | E | 111 | −10.262 | 76.009 | −24.972 | 1.00 | 199.97 |
| 7003 | O | ARG | E | 111 | −10.388 | 77.173 | −25.350 | 1.00 | 199.97 |
| 7004 | N | ASN | E | 112 | −10.597 | 75.590 | −23.767 | 1.00 | 112.19 |
| 7005 | CA | ASN | E | 112 | −11.129 | 76.482 | −22.769 | 1.00 | 112.19 |
| 7006 | CB | ASN | E | 112 | −10.101 | 77.553 | −22.432 | 1.00 | 133.79 |
| 7007 | CG | ASN | E | 112 | −10.330 | 78.144 | −21.061 | 1.00 | 133.79 |
| 7008 | OD1 | ASN | E | 112 | −11.446 | 78.097 | −20.531 | 1.00 | 133.79 |
| 7009 | ND2 | ASN | E | 112 | −9.280 | 78.710 | −20.478 | 1.00 | 133.79 |
| 7010 | C | ASN | E | 112 | −12.445 | 77.147 | −23.163 | 1.00 | 112.19 |
| 7011 | O | ASN | E | 112 | −12.776 | 78.218 | −22.651 | 1.00 | 112.19 |
| 7012 | N | TRP | E | 113 | −13.200 | 76.532 | −24.067 | 1.00 | 154.00 |
| 7013 | CA | TRP | E | 113 | −14.478 | 77.116 | −24.441 | 1.00 | 154.00 |
| 7014 | CB | TRP | E | 113 | −15.153 | 76.326 | −25.542 | 1.00 | 235.27 |
| 7015 | CG | TRP | E | 113 | −14.586 | 76.595 | −26.860 | 1.00 | 235.27 |
| 7016 | CD2 | TRP | E | 113 | −14.400 | 75.651 | −27.909 | 1.00 | 235.27 |
| 7017 | CE2 | TRP | E | 113 | −13.885 | 76.353 | −29.018 | 1.00 | 235.27 |
| 7018 | CE3 | TRP | E | 113 | −14.628 | 74.270 | −28.027 | 1.00 | 235.27 |
| 7019 | CD1 | TRP | E | 113 | −14.180 | 77.804 | −27.348 | 1.00 | 235.27 |
| 7020 | NE1 | TRP | E | 113 | −13.758 | 77.667 | −28.647 | 1.00 | 235.27 |
| 7021 | CZ2 | TRP | E | 113 | −13.592 | 75.726 | −30.229 | 1.00 | 235.27 |
| 7022 | CZ3 | TRP | E | 113 | −14.335 | 73.643 | −29.233 | 1.00 | 235.27 |
| 7023 | CH2 | TRP | E | 113 | −13.818 | 74.373 | −30.318 | 1.00 | 235.27 |
| 7024 | C | TRP | E | 113 | −15.400 | 77.133 | −23.246 | 1.00 | 154.00 |
| 7025 | O | TRP | E | 113 | −14.983 | 76.886 | −22.116 | 1.00 | 154.00 |
| 7026 | N | ASP | E | 114 | −16.664 | 77.430 | −23.503 | 1.00 | 242.58 |
| 7027 | CA | ASP | E | 114 | −17.649 | 77.470 | −22.442 | 1.00 | 242.58 |
| 7028 | CB | ASP | E | 114 | −18.418 | 78.794 | −22.471 | 1.00 | 249.32 |
| 7029 | CG | ASP | E | 114 | −17.656 | 79.922 | −21.803 | 1.00 | 249.32 |
| 7030 | OD1 | ASP | E | 114 | −17.348 | 79.791 | −20.599 | 1.00 | 249.32 |
| 7031 | OD2 | ASP | E | 114 | −17.365 | 80.933 | −22.475 | 1.00 | 249.32 |
| 7032 | C | ASP | E | 114 | −18.606 | 76.306 | −22.572 | 1.00 | 242.58 |
| 7033 | O | ASP | E | 114 | −19.027 | 75.942 | −23.672 | 1.00 | 242.58 |
| 7034 | N | VAL | E | 115 | −18.931 | 75.717 | −21.430 | 1.00 | 148.92 |
| 7035 | CA | VAL | E | 115 | −19.846 | 74.594 | −21.391 | 1.00 | 148.92 |
| 7036 | CB | VAL | E | 115 | −19.199 | 73.377 | −20.746 | 1.00 | 243.92 |
| 7037 | CG1 | VAL | E | 115 | −20.086 | 72.162 | −20.949 | 1.00 | 243.92 |
| 7038 | CG2 | VAL | E | 115 | −17.821 | 73.161 | −21.331 | 1.00 | 243.92 |
| 7039 | C | VAL | E | 115 | −21.075 | 74.959 | −20.581 | 1.00 | 148.92 |
| 7040 | O | VAL | E | 115 | −20.985 | 75.672 | −19.577 | 1.00 | 148.92 |
| 7041 | N | TYR | E | 116 | −22.226 | 74.466 | −21.020 | 1.00 | 86.50 |
| 7042 | CA | TYR | E | 116 | −23.470 | 74.752 | −20.320 | 1.00 | 86.50 |
| 7043 | CB | TYR | E | 116 | −24.374 | 75.633 | −21.192 | 1.00 | 249.77 |
| 7044 | CG | TYR | E | 116 | −23.782 | 76.991 | −21.517 | 1.00 | 249.77 |
| 7045 | CD1 | TYR | E | 116 | −23.088 | 77.210 | −22.709 | 1.00 | 249.77 |
| 7046 | CE1 | TYR | E | 116 | −22.513 | 78.454 | −22.994 | 1.00 | 249.77 |
| 7047 | CD2 | TYR | E | 116 | −23.889 | 78.048 | −20.616 | 1.00 | 249.77 |
| 7048 | CE2 | TYR | E | 116 | −23.319 | 79.293 | −20.886 | 1.00 | 249.77 |
| 7049 | CZ | TYR | E | 116 | −22.631 | 79.488 | −22.075 | 1.00 | 249.77 |
| 7050 | OH | TYR | E | 116 | −22.051 | 80.710 | −22.336 | 1.00 | 249.77 |
| 7051 | C | TYR | E | 116 | −24.208 | 73.466 | −19.940 | 1.00 | 86.50 |
| 7052 | O | TYR | E | 116 | −23.829 | 72.356 | −20.362 | 1.00 | 86.50 |
| 7053 | N | LYS | E | 117 | −25.277 | 73.630 | −19.164 | 1.00 | 217.77 |
| 7054 | CA | LYS | E | 117 | −26.078 | 72.500 | −18.707 | 1.00 | 217.77 |
| 7055 | CB | LYS | E | 117 | −26.963 | 71.967 | −19.832 | 1.00 | 191.92 |
| 7056 | CG | LYS | E | 117 | −28.295 | 72.674 | −19.997 | 1.00 | 191.92 |
| 7057 | CD | LYS | E | 117 | −29.246 | 71.813 | −20.819 | 1.00 | 191.92 |
| 7058 | CE | LYS | E | 117 | −29.481 | 70.462 | −20.143 | 1.00 | 191.92 |
| 7059 | NZ | LYS | E | 117 | −30.376 | 69.560 | −20.915 | 1.00 | 191.92 |
| 7060 | C | LYS | E | 117 | −25.161 | 71.387 | −18.230 | 1.00 | 217.77 |
| 7061 | O | LYS | E | 117 | −25.228 | 70.262 | −18.724 | 1.00 | 217.77 |
| 7062 | N | VAL | E | 118 | −24.306 | 71.709 | −17.268 | 1.00 | 181.28 |
| 7063 | CA | VAL | E | 118 | −23.356 | 70.743 | −16.731 | 1.00 | 181.28 |
| 7064 | CB | VAL | E | 118 | −22.089 | 71.444 | −16.254 | 1.00 | 157.61 |
| 7065 | CG1 | VAL | E | 118 | −21.427 | 70.628 | −15.171 | 1.00 | 157.61 |
| 7066 | CG2 | VAL | E | 118 | −21.141 | 71.619 | −17.420 | 1.00 | 157.61 |
| 7067 | C | VAL | E | 118 | −23.857 | 69.864 | −15.598 | 1.00 | 181.28 |
| 7068 | O | VAL | E | 118 | −24.500 | 70.335 | −14.661 | 1.00 | 181.28 |
| 7069 | N | ILE | E | 119 | −23.514 | 68.585 | −15.674 | 1.00 | 95.37 |
| 7070 | CA | ILE | E | 119 | −23.932 | 67.630 | −14.665 | 1.00 | 95.37 |
| 7071 | CB | ILE | E | 119 | −25.093 | 66.783 | −15.184 | 1.00 | 80.86 |
| 7072 | CG2 | ILE | E | 119 | −25.598 | 65.858 | −14.102 | 1.00 | 80.86 |
| 7073 | CG1 | ILE | E | 119 | −26.198 | 67.695 | −15.696 | 1.00 | 80.86 |
| 7074 | CD1 | ILE | E | 119 | −27.227 | 66.956 | −16.527 | 1.00 | 80.86 |
| 7075 | C | ILE | E | 119 | −22.791 | 66.678 | −14.348 | 1.00 | 95.37 |
| 7076 | O | ILE | E | 119 | −22.280 | 66.017 | −15.249 | 1.00 | 95.37 |
| 7077 | N | TYR | E | 120 | −22.373 | 66.602 | −13.088 | 1.00 | 103.71 |
| 7078 | CA | TYR | E | 120 | −21.315 | 65.664 | −12.732 | 1.00 | 103.71 |
| 7079 | CB | TYR | E | 120 | −20.499 | 66.128 | −11.550 | 1.00 | 87.63 |
| 7080 | CG | TYR | E | 120 | −19.634 | 67.303 | −11.821 | 1.00 | 87.63 |
| 7081 | CD1 | TYR | E | 120 | −20.141 | 68.575 | −11.746 | 1.00 | 87.63 |
| 7082 | CE1 | TYR | E | 120 | −19.351 | 69.666 | −12.010 | 1.00 | 87.63 |
| 7083 | CD2 | TYR | E | 120 | −18.300 | 67.141 | −12.171 | 1.00 | 87.63 |
| 7084 | CE2 | TYR | E | 120 | −17.486 | 68.226 | −12.447 | 1.00 | 87.63 |
| 7085 | CZ | TYR | E | 120 | −18.017 | 69.490 | −12.366 | 1.00 | 87.63 |
| 7086 | OH | TYR | E | 120 | −17.220 | 70.579 | −12.651 | 1.00 | 87.63 |
| 7087 | C | TYR | E | 120 | −21.972 | 64.380 | −12.305 | 1.00 | 103.71 |
| 7088 | O | TYR | E | 120 | −23.037 | 64.401 | −11.694 | 1.00 | 103.71 |
| 7089 | N | TYR | E | 121 | −21.324 | 63.263 | −12.596 | 1.00 | 62.69 |
| 7090 | CA | TYR | E | 121 | −21.857 | 61.963 | −12.221 | 1.00 | 62.69 |
| 7091 | CB | TYR | E | 121 | −22.202 | 61.148 | −13.476 | 1.00 | 95.66 |
| 7092 | CG | TYR | E | 121 | −23.364 | 61.653 | −14.309 | 1.00 | 95.66 |
| 7093 | CD1 | TYR | E | 121 | −23.288 | 62.865 | −14.992 | 1.00 | 95.66 |
| 7094 | CE1 | TYR | E | 121 | −24.334 | 63.305 | −15.806 | 1.00 | 95.66 |
| 7095 | CD2 | TYR | E | 121 | −24.522 | 60.885 | −14.455 | 1.00 | 95.66 |
| 7096 | CE2 | TYR | E | 121 | −25.576 | 61.315 | −15.269 | 1.00 | 95.66 |
| 7097 | CZ | TYR | E | 121 | −25.477 | 62.527 | −15.945 | 1.00 | 95.66 |
| 7098 | OH | TYR | E | 121 | −26.515 | 62.943 | −16.761 | 1.00 | 95.66 |
| 7099 | C | TYR | E | 121 | −20.873 | 61.165 | −11.368 | 1.00 | 62.69 |
| 7100 | O | TYR | E | 121 | −19.667 | 61.179 | −11.620 | 1.00 | 62.69 |
| 7101 | N | LYS | E | 122 | −21.391 | 60.478 | −10.356 | 1.00 | 76.05 |
| 7102 | CA | LYS | E | 122 | −20.562 | 59.633 | −9.521 | 1.00 | 76.05 |
| 7103 | CB | LYS | E | 122 | −20.410 | 60.198 | −8.114 | 1.00 | 107.43 |
| 7104 | CG | LYS | E | 122 | −19.516 | 59.339 | −7.238 | 1.00 | 107.43 |
| 7105 | CD | LYS | E | 122 | −19.635 | 59.698 | −5.779 | 1.00 | 107.43 |
| 7106 | CE | LYS | E | 122 | −18.887 | 58.706 | −4.914 | 1.00 | 107.43 |
| 7107 | NZ | LYS | E | 122 | −19.161 | 58.998 | −3.484 | 1.00 | 107.43 |
| 7108 | C | LYS | E | 122 | −21.223 | 58.263 | −9.440 | 1.00 | 76.05 |
| 7109 | O | LYS | E | 122 | −22.325 | 58.127 | −8.898 | 1.00 | 76.05 |
| 7110 | N | ASP | E | 123 | −20.543 | 57.252 | −9.973 | 1.00 | 138.97 |
| 7111 | CA | ASP | E | 123 | −21.059 | 55.892 | −9.976 | 1.00 | 138.97 |
| 7112 | CB | ASP | E | 123 | −21.188 | 55.363 | −8.545 | 1.00 | 185.30 |
| 7113 | CG | ASP | E | 123 | −19.849 | 54.991 | −7.945 | 1.00 | 185.30 |
| 7114 | OD1 | ASP | E | 123 | −19.068 | 54.303 | −8.638 | 1.00 | 185.30 |
| 7115 | OD2 | ASP | E | 123 | −19.578 | 55.374 | −6.785 | 1.00 | 185.30 |
| 7116 | C | ASP | E | 123 | −22.396 | 55.792 | −10.702 | 1.00 | 138.97 |
| 7117 | O | ASP | E | 123 | −23.333 | 55.147 | −10.225 | 1.00 | 138.97 |
| 7118 | N | GLY | E | 124 | −22.472 | 56.443 | −11.862 | 1.00 | 163.35 |
| 7119 | CA | GLY | E | 124 | −23.675 | 56.412 | −12.675 | 1.00 | 163.35 |
| 7120 | C | GLY | E | 124 | −24.838 | 57.262 | −12.200 | 1.00 | 163.35 |
| 7121 | O | GLY | E | 124 | −25.840 | 57.388 | −12.907 | 1.00 | 163.35 |
| 7122 | N | GLU | E | 125 | −24.711 | 57.854 | −11.016 | 1.00 | 131.17 |
| 7123 | CA | GLU | E | 125 | −25.777 | 58.684 | −10.447 | 1.00 | 131.17 |
| 7124 | CB | GLU | E | 125 | −25.822 | 58.535 | −8.911 | 1.00 | 143.41 |
| 7125 | CG | GLU | E | 125 | −26.269 | 57.168 | −8.368 | 1.00 | 143.41 |
| 7126 | CD | GLU | E | 125 | −27.780 | 56.971 | −8.384 | 1.00 | 143.41 |
| 7127 | OE1 | GLU | E | 125 | −28.492 | 57.744 | −7.700 | 1.00 | 143.41 |
| 7128 | OE2 | GLU | E | 125 | −28.249 | 56.040 | −9.077 | 1.00 | 143.41 |
| 7129 | C | GLU | E | 125 | −25.597 | 60.160 | −10.771 | 1.00 | 131.17 |
| 7130 | O | GLU | E | 125 | −24.483 | 60.656 | −10.809 | 1.00 | 131.17 |
| 7131 | N | ALA | E | 126 | −26.695 | 60.864 | −11.006 | 1.00 | 115.32 |
| 7132 | CA | ALA | E | 126 | −26.604 | 62.290 | −11.265 | 1.00 | 115.32 |
| 7133 | CB | ALA | E | 126 | −27.979 | 62.844 | −11.605 | 1.00 | 168.61 |
| 7134 | C | ALA | E | 126 | −26.104 | 62.855 | −9.935 | 1.00 | 115.32 |
| 7135 | O | ALA | E | 126 | −26.467 | 62.339 | −8.876 | 1.00 | 115.32 |
| 7136 | N | LEU | E | 127 | −25.275 | 63.897 | −9.967 | 1.00 | 118.41 |
| 7137 | CA | LEU | E | 127 | −24.752 | 64.458 | −8.714 | 1.00 | 118.41 |
| 7138 | CB | LEU | E | 127 | −23.271 | 64.162 | −8.577 | 1.00 | 111.80 |
| 7139 | CG | LEU | E | 127 | −22.934 | 64.190 | −7.090 | 1.00 | 111.80 |
| 7140 | CD1 | LEU | E | 127 | −23.811 | 63.158 | −6.384 | 1.00 | 111.80 |
| 7141 | CD2 | LEU | E | 127 | −21.465 | 63.889 | −6.868 | 1.00 | 111.80 |
| 7142 | C | LEU | E | 127 | −24.957 | 65.947 | −8.453 | 1.00 | 118.41 |
| 7143 | O | LEU | E | 127 | −25.470 | 66.328 | −7.404 | 1.00 | 118.41 |
| 7144 | N | LYS | E | 128 | −24.498 | 66.785 | −9.373 | 1.00 | 133.41 |
| 7145 | CA | LYS | E | 128 | −24.677 | 68.228 | −9.259 | 1.00 | 133.41 |
| 7146 | CB | LYS | E | 128 | −23.405 | 68.893 | −8.760 | 1.00 | 171.72 |
| 7147 | CG | LYS | E | 128 | −22.965 | 68.445 | −7.388 | 1.00 | 171.72 |
| 7148 | CD | LYS | E | 128 | −23.865 | 68.977 | −6.284 | 1.00 | 171.72 |
| 7149 | CE | LYS | E | 128 | −23.287 | 68.616 | −4.917 | 1.00 | 171.72 |
| 7150 | NZ | LYS | E | 128 | −24.024 | 69.227 | −3.779 | 1.00 | 171.72 |
| 7151 | C | LYS | E | 128 | −25.015 | 68.757 | −10.653 | 1.00 | 133.41 |
| 7152 | O | LYS | E | 128 | −24.626 | 68.153 | −11.657 | 1.00 | 133.41 |
| 7153 | N | TYR | E | 129 | −25.733 | 69.876 | −10.729 | 1.00 | 159.58 |
| 7154 | CA | TYR | E | 129 | −26.106 | 70.442 | −12.029 | 1.00 | 159.58 |
| 7155 | CB | TYR | E | 129 | −27.496 | 69.983 | −12.438 | 1.00 | 246.12 |
| 7156 | CG | TYR | E | 129 | −28.122 | 70.887 | −13.441 | 1.00 | 246.12 |
| 7157 | CD1 | TYR | E | 129 | −27.756 | 70.809 | −14.781 | 1.00 | 246.12 |
| 7158 | CE1 | TYR | E | 129 | −28.298 | 71.675 | −15.715 | 1.00 | 246.12 |
| 7159 | CD2 | TYR | E | 129 | −29.063 | 71.857 | −13.055 | 1.00 | 246.12 |
| 7160 | CE2 | TYR | E | 129 | −29.624 | 72.721 | −13.990 | 1.00 | 246.12 |
| 7161 | CZ | TYR | E | 129 | −29.236 | 72.624 | −15.326 | 1.00 | 246.12 |
| 7162 | OH | TYR | E | 129 | −29.822 | 73.442 | −16.274 | 1.00 | 246.12 |
| 7163 | C | TYR | E | 129 | −26.106 | 71.953 | −12.048 | 1.00 | 159.58 |
| 7164 | O | TYR | E | 129 | −26.579 | 72.589 | −11.112 | 1.00 | 159.58 |
| 7165 | N | TRP | E | 130 | −25.600 | 72.526 | −13.137 | 1.00 | 184.49 |
| 7166 | CA | TRP | E | 130 | −25.557 | 73.976 | −13.280 | 1.00 | 184.49 |
| 7167 | CB | TRP | E | 130 | −24.211 | 74.535 | −12.817 | 1.00 | 245.42 |
| 7168 | CG | TRP | E | 130 | −23.751 | 74.067 | −11.472 | 1.00 | 245.42 |
| 7169 | CD2 | TRP | E | 130 | −23.750 | 74.828 | −10.257 | 1.00 | 245.42 |
| 7170 | CE2 | TRP | E | 130 | −23.186 | 74.005 | −9.254 | 1.00 | 245.42 |
| 7171 | CE3 | TRP | E | 130 | −24.179 | 76.119 | −9.918 | 1.00 | 245.42 |
| 7172 | CD1 | TRP | E | 130 | −23.202 | 72.856 | −11.166 | 1.00 | 245.42 |
| 7173 | NE1 | TRP | E | 130 | −22.859 | 72.811 | −9.835 | 1.00 | 245.42 |
| 7174 | CZ2 | TRP | E | 130 | −23.034 | 74.438 | −7.930 | 1.00 | 245.42 |
| 7175 | CZ3 | TRP | E | 130 | −24.028 | 76.548 | −8.600 | 1.00 | 245.42 |
| 7176 | CH2 | TRP | E | 130 | −23.454 | 75.709 | −7.626 | 1.00 | 245.42 |
| 7177 | C | TRP | E | 130 | −25.768 | 74.407 | −14.725 | 1.00 | 184.49 |
| 7178 | O | TRP | E | 130 | −25.711 | 73.584 | −15.638 | 1.00 | 184.49 |
| 7179 | N | TYR | E | 131 | −26.014 | 75.701 | −14.930 | 1.00 | 185.41 |
| 7180 | CA | TYR | E | 131 | −26.187 | 76.228 | −16.277 | 1.00 | 185.41 |
| 7181 | CB | TYR | E | 131 | −27.063 | 77.477 | −16.282 | 1.00 | 249.42 |
| 7182 | CG | TYR | E | 131 | −27.438 | 77.858 | −17.685 | 1.00 | 249.42 |
| 7183 | CD1 | TYR | E | 131 | −28.391 | 77.125 | −18.389 | 1.00 | 249.42 |
| 7184 | CE1 | TYR | E | 131 | −28.654 | 77.384 | −19.728 | 1.00 | 249.42 |
| 7185 | CD2 | TYR | E | 131 | −26.755 | 78.869 | −18.358 | 1.00 | 249.42 |
| 7186 | CE2 | TYR | E | 131 | −26.997 | 79.127 | −19.703 | 1.00 | 249.42 |
| 7187 | CZ | TYR | E | 131 | −27.951 | 78.384 | −20.380 | 1.00 | 249.42 |
| 7188 | OH | TYR | E | 131 | −28.189 | 78.642 | −21.710 | 1.00 | 249.42 |
| 7189 | C | TYR | E | 131 | −24.780 | 76.577 | −16.758 | 1.00 | 185.41 |
| 7190 | O | TYR | E | 131 | −24.141 | 75.784 | −17.455 | 1.00 | 185.41 |
| 7191 | N | GLU | E | 132 | −24.310 | 77.777 | −16.415 | 1.00 | 229.70 |
| 7192 | CA | GLU | E | 132 | −22.942 | 78.159 | −16.751 | 1.00 | 229.70 |
| 7193 | CB | GLU | E | 132 | −22.638 | 79.608 | −16.344 | 1.00 | 249.20 |
| 7194 | CG | GLU | E | 132 | −23.207 | 80.700 | −17.258 | 1.00 | 249.20 |
| 7195 | CD | GLU | E | 132 | −22.123 | 81.565 | −17.898 | 1.00 | 249.20 |
| 7196 | OE1 | GLU | E | 132 | −20.982 | 81.562 | −17.392 | 1.00 | 249.20 |
| 7197 | OE2 | GLU | E | 132 | −22.412 | 82.255 | −18.899 | 1.00 | 249.20 |
| 7198 | C | GLU | E | 132 | −22.290 | 77.200 | −15.773 | 1.00 | 229.70 |
| 7199 | O | GLU | E | 132 | −22.652 | 77.190 | −14.595 | 1.00 | 229.70 |
| 7200 | N | ASN | E | 133 | −21.348 | 76.385 | −16.233 | 1.00 | 219.94 |
| 7201 | CA | ASN | E | 133 | −20.764 | 75.401 | −15.332 | 1.00 | 219.94 |
| 7202 | CB | ASN | E | 133 | −19.878 | 74.415 | −16.095 | 1.00 | 129.08 |
| 7203 | CG | ASN | E | 133 | −18.469 | 74.898 | −16.252 | 1.00 | 129.08 |
| 7204 | OD1 | ASN | E | 133 | −18.235 | 76.020 | −16.692 | 1.00 | 129.08 |
| 7205 | ND2 | ASN | E | 133 | −17.510 | 74.046 | −15.904 | 1.00 | 129.08 |
| 7206 | C | ASN | E | 133 | −20.023 | 75.919 | −14.118 | 1.00 | 219.94 |
| 7207 | O | ASN | E | 133 | −19.802 | 77.115 | −13.944 | 1.00 | 219.94 |
| 7208 | N | HIS | E | 134 | −19.638 | 74.965 | −13.286 | 1.00 | 192.14 |
| 7209 | CA | HIS | E | 134 | −18.970 | 75.231 | −12.036 | 1.00 | 192.14 |
| 7210 | CB | HIS | E | 134 | −20.007 | 75.079 | −10.924 | 1.00 | 214.14 |
| 7211 | CG | HIS | E | 134 | −19.514 | 75.463 | −9.567 | 1.00 | 214.14 |
| 7212 | CD2 | HIS | E | 134 | −19.356 | 74.731 | −8.436 | 1.00 | 214.14 |
| 7213 | ND1 | HIS | E | 134 | −19.138 | 76.749 | −9.245 | 1.00 | 214.14 |
| 7214 | CE1 | HIS | E | 134 | −18.771 | 76.794 | −7.976 | 1.00 | 214.14 |
| 7215 | NE2 | HIS | E | 134 | −18.895 | 75.584 | −7.463 | 1.00 | 214.14 |
| 7216 | C | HIS | E | 134 | −17.828 | 74.231 | −11.860 | 1.00 | 192.14 |
| 7217 | O | HIS | E | 134 | −17.412 | 73.574 | −12.820 | 1.00 | 192.14 |
| 7218 | N | ASN | E | 135 | −17.336 | 74.114 | −10.630 | 1.00 | 109.49 |
| 7219 | CA | ASN | E | 135 | −16.246 | 73.208 | −10.311 | 1.00 | 109.49 |
| 7220 | CB | ASN | E | 135 | −14.921 | 73.967 | −10.346 | 1.00 | 216.32 |
| 7221 | CG | ASN | E | 135 | −14.571 | 74.433 | −11.741 | 1.00 | 216.32 |
| 7222 | OD1 | ASN | E | 135 | −14.694 | 73.661 | −12.691 | 1.00 | 216.32 |
| 7223 | ND2 | ASN | E | 135 | −14.125 | 75.677 | −11.883 | 1.00 | 216.32 |
| 7224 | C | ASN | E | 135 | −16.462 | 72.573 | −8.957 | 1.00 | 109.49 |
| 7225 | O | ASN | E | 135 | −15.960 | 73.058 | −7.948 | 1.00 | 109.49 |
| 7226 | N | ILE | E | 136 | −17.223 | 71.484 | −8.949 | 1.00 | 98.56 |
| 7227 | CA | ILE | E | 136 | −17.541 | 70.753 | −7.725 | 1.00 | 98.56 |
| 7228 | CB | ILE | E | 136 | −18.026 | 69.322 | −8.063 | 1.00 | 164.42 |
| 7229 | CG2 | ILE | E | 136 | −17.070 | 68.654 | −9.020 | 1.00 | 164.42 |
| 7230 | CG1 | ILE | E | 136 | −18.182 | 68.505 | −6.792 | 1.00 | 164.42 |
| 7231 | CD1 | ILE | E | 136 | −18.771 | 67.146 | −7.055 | 1.00 | 164.42 |
| 7232 | C | ILE | E | 136 | −16.351 | 70.717 | −6.779 | 1.00 | 98.56 |
| 7233 | O | ILE | E | 136 | −15.300 | 70.168 | −7.104 | 1.00 | 98.56 |
| 7234 | N | SER | E | 137 | −16.524 | 71.329 | −5.613 | 1.00 | 114.80 |
| 7235 | CA | SER | E | 137 | −15.462 | 71.405 | −4.622 | 1.00 | 114.80 |
| 7236 | CB | SER | E | 137 | −15.128 | 72.866 | −4.360 | 1.00 | 96.11 |
| 7237 | OG | SER | E | 137 | −14.336 | 72.997 | −3.196 | 1.00 | 96.11 |
| 7238 | C | SER | E | 137 | −15.798 | 70.714 | −3.303 | 1.00 | 114.80 |
| 7239 | O | SER | E | 137 | −16.955 | 70.690 | −2.879 | 1.00 | 114.80 |
| 7240 | N | ILE | E | 138 | −14.773 | 70.169 | −2.652 | 1.00 | 105.32 |
| 7241 | CA | ILE | E | 138 | −14.939 | 69.466 | −1.382 | 1.00 | 105.32 |
| 7242 | CB | ILE | E | 138 | −14.851 | 67.969 | −1.577 | 1.00 | 81.88 |
| 7243 | CG2 | ILE | E | 138 | −14.767 | 67.296 | −0.223 | 1.00 | 81.88 |
| 7244 | CG1 | ILE | E | 138 | −16.049 | 67.484 | −2.408 | 1.00 | 81.88 |
| 7245 | CD1 | ILE | E | 138 | −15.917 | 66.048 | −2.912 | 1.00 | 81.88 |
| 7246 | C | ILE | E | 138 | −13.869 | 69.833 | −0.374 | 1.00 | 105.32 |
| 7247 | O | ILE | E | 138 | −12.686 | 69.614 | −0.619 | 1.00 | 105.32 |
| 7248 | N | THR | E | 139 | −14.283 | 70.362 | 0.772 | 1.00 | 128.39 |
| 7249 | CA | THR | E | 139 | −13.333 | 70.758 | 1.807 | 1.00 | 128.39 |
| 7250 | CB | THR | E | 139 | −13.986 | 71.743 | 2.788 | 1.00 | 173.94 |
| 7251 | OG1 | THR | E | 139 | −15.220 | 71.194 | 3.265 | 1.00 | 173.94 |
| 7252 | CG2 | THR | E | 139 | −14.267 | 73.071 | 2.094 | 1.00 | 173.94 |
| 7253 | C | THR | E | 139 | −12.825 | 69.535 | 2.566 | 1.00 | 128.39 |
| 7254 | O | THR | E | 139 | −11.709 | 69.058 | 2.337 | 1.00 | 128.39 |
| 7255 | N | ASN | E | 140 | −13.650 | 69.041 | 3.480 | 1.00 | 224.25 |
| 7256 | CA | ASN | E | 140 | −13.323 | 67.859 | 4.263 | 1.00 | 224.25 |
| 7257 | CB | ASN | E | 140 | −14.012 | 67.918 | 5.625 | 1.00 | 231.48 |
| 7258 | CG | ASN | E | 140 | −13.813 | 66.656 | 6.429 | 1.00 | 231.48 |
| 7259 | OD1 | ASN | E | 140 | −13.998 | 65.547 | 5.923 | 1.00 | 231.48 |
| 7260 | ND2 | ASN | E | 140 | −13.443 | 66.823 | 7.692 | 1.00 | 231.48 |
| 7261 | C | ASN | E | 140 | −13.874 | 66.691 | 3.464 | 1.00 | 224.25 |
| 7262 | O | ASN | E | 140 | −15.073 | 66.629 | 3.204 | 1.00 | 224.25 |
| 7263 | N | ALA | E | 141 | −13.004 | 65.763 | 3.086 | 1.00 | 132.13 |
| 7264 | CA | ALA | E | 141 | −13.420 | 64.623 | 2.289 | 1.00 | 132.13 |
| 7265 | CB | ALA | E | 141 | −12.374 | 64.323 | 1.257 | 1.00 | 95.25 |
| 7266 | C | ALA | E | 141 | −13.730 | 63.367 | 3.072 | 1.00 | 132.13 |
| 7267 | O | ALA | E | 141 | −12.932 | 62.901 | 3.884 | 1.00 | 132.13 |
| 7268 | N | THR | E | 142 | −14.903 | 62.811 | 2.802 | 1.00 | 103.70 |
| 7269 | CA | THR | E | 142 | −15.339 | 61.588 | 3.454 | 1.00 | 103.70 |
| 7270 | CB | THR | E | 142 | −16.873 | 61.526 | 3.477 | 1.00 | 152.79 |
| 7271 | OG1 | THR | E | 142 | −17.384 | 62.747 | 4.028 | 1.00 | 152.79 |
| 7272 | CG2 | THR | E | 142 | −17.346 | 60.376 | 4.329 | 1.00 | 152.79 |
| 7273 | C | THR | E | 142 | −14.767 | 60.417 | 2.650 | 1.00 | 103.70 |
| 7274 | O | THR | E | 142 | −14.198 | 60.624 | 1.575 | 1.00 | 103.70 |
| 7275 | N | VAL | E | 143 | −14.887 | 59.197 | 3.164 | 1.00 | 124.54 |
| 7276 | CA | VAL | E | 143 | −14.369 | 58.042 | 2.437 | 1.00 | 124.54 |
| 7277 | CB | VAL | E | 143 | −14.026 | 56.863 | 3.365 | 1.00 | 132.81 |
| 7278 | CG1 | VAL | E | 143 | −15.292 | 56.315 | 3.999 | 1.00 | 132.81 |
| 7279 | CG2 | VAL | E | 143 | −13.310 | 55.773 | 2.577 | 1.00 | 132.81 |
| 7280 | C | VAL | E | 143 | −15.448 | 57.583 | 1.484 | 1.00 | 124.54 |
| 7281 | O | VAL | E | 143 | −15.187 | 56.822 | 0.555 | 1.00 | 124.54 |
| 7282 | N | GLU | E | 144 | −16.668 | 58.047 | 1.722 | 1.00 | 123.03 |
| 7283 | CA | GLU | E | 144 | −17.781 | 57.678 | 0.863 | 1.00 | 123.03 |
| 7284 | CB | GLU | E | 144 | −19.108 | 57.964 | 1.558 | 1.00 | 249.45 |
| 7285 | CG | GLU | E | 144 | −19.323 | 57.136 | 2.812 | 1.00 | 249.45 |
| 7286 | CD | GLU | E | 144 | −19.316 | 57.978 | 4.073 | 1.00 | 249.45 |
| 7287 | OE1 | GLU | E | 144 | −20.163 | 58.892 | 4.176 | 1.00 | 249.45 |
| 7288 | OE2 | GLU | E | 144 | −18.470 | 57.730 | 4.958 | 1.00 | 249.45 |
| 7289 | C | GLU | E | 144 | −17.709 | 58.428 | −0.460 | 1.00 | 123.03 |
| 7290 | O | GLU | E | 144 | −18.373 | 58.051 | −1.416 | 1.00 | 123.03 |
| 7291 | N | ASP | E | 145 | −16.897 | 59.485 | −0.509 | 1.00 | 78.25 |
| 7292 | CA | ASP | E | 145 | −16.721 | 60.284 | −1.726 | 1.00 | 78.25 |
| 7293 | CB | ASP | E | 145 | −16.017 | 61.593 | −1.396 | 1.00 | 126.03 |
| 7294 | CG | ASP | E | 145 | −16.912 | 62.551 | −0.679 | 1.00 | 126.03 |
| 7295 | OD1 | ASP | E | 145 | −17.955 | 62.900 | −1.272 | 1.00 | 126.03 |
| 7296 | OD2 | ASP | E | 145 | −16.581 | 62.944 | 0.465 | 1.00 | 126.03 |
| 7297 | C | ASP | E | 145 | −15.918 | 59.528 | −2.789 | 1.00 | 78.25 |
| 7298 | O | ASP | E | 145 | −15.889 | 59.920 | −3.952 | 1.00 | 78.25 |
| 7299 | N | SER | E | 146 | −15.263 | 58.442 | −2.388 | 1.00 | 91.78 |
| 7300 | CA | SER | E | 146 | −14.482 | 57.660 | −3.331 | 1.00 | 91.78 |
| 7301 | CB | SER | E | 146 | −13.708 | 56.568 | −2.591 | 1.00 | 200.20 |
| 7302 | OG | SER | E | 146 | −12.805 | 57.145 | −1.661 | 1.00 | 200.20 |
| 7303 | C | SER | E | 146 | −15.448 | 57.065 | −4.335 | 1.00 | 91.78 |
| 7304 | O | SER | E | 146 | −16.558 | 56.675 | −3.981 | 1.00 | 91.78 |
| 7305 | N | GLY | E | 147 | −15.039 | 57.020 | −5.592 | 1.00 | 97.94 |
| 7306 | CA | GLY | E | 147 | −15.910 | 56.484 | −6.622 | 1.00 | 97.94 |
| 7307 | C | GLY | E | 147 | −15.365 | 56.777 | −8.007 | 1.00 | 97.94 |
| 7308 | O | GLY | E | 147 | −14.177 | 57.079 | −8.146 | 1.00 | 97.94 |
| 7309 | N | THR | E | 148 | −16.211 | 56.677 | −9.033 | 1.00 | 66.84 |
| 7310 | CA | THR | E | 148 | −15.767 | 56.955 | −10.399 | 1.00 | 66.84 |
| 7311 | CB | THR | E | 148 | −15.794 | 55.674 | −11.285 | 1.00 | 76.59 |
| 7312 | OG1 | THR | E | 148 | −16.932 | 55.699 | −12.141 | 1.00 | 76.59 |
| 7313 | CG2 | THR | E | 148 | −15.880 | 54.450 | −10.424 | 1.00 | 76.59 |
| 7314 | C | THR | E | 148 | −16.662 | 58.055 | −10.978 | 1.00 | 66.84 |
| 7315 | O | THR | E | 148 | −17.884 | 57.889 | −11.145 | 1.00 | 66.84 |
| 7316 | N | TYR | E | 149 | −16.041 | 59.187 | −11.273 | 1.00 | 55.84 |
| 7317 | CA | TYR | E | 149 | −16.768 | 60.335 | −11.774 | 1.00 | 55.84 |
| 7318 | CB | TYR | E | 149 | −16.262 | 61.585 | −11.070 | 1.00 | 63.87 |
| 7319 | CG | TYR | E | 149 | −16.445 | 61.611 | −9.575 | 1.00 | 63.87 |
| 7320 | CD1 | TYR | E | 149 | −15.728 | 60.766 | −8.730 | 1.00 | 63.87 |
| 7321 | CE1 | TYR | E | 149 | −15.895 | 60.837 | −7.342 | 1.00 | 63.87 |
| 7322 | CD2 | TYR | E | 149 | −17.326 | 62.515 | −9.003 | 1.00 | 63.87 |
| 7323 | CE2 | TYR | E | 149 | −17.502 | 62.597 | −7.639 | 1.00 | 63.87 |
| 7324 | CZ | TYR | E | 149 | −16.793 | 61.761 | −6.812 | 1.00 | 63.87 |
| 7325 | OH | TYR | E | 149 | −17.022 | 61.871 | −5.455 | 1.00 | 63.87 |
| 7326 | C | TYR | E | 149 | −16.630 | 60.541 | −13.274 | 1.00 | 55.84 |
| 7327 | O | TYR | E | 149 | −15.789 | 59.909 | −13.918 | 1.00 | 55.84 |
| 7328 | N | TYR | E | 150 | −17.478 | 61.414 | −13.817 | 1.00 | 90.25 |
| 7329 | CA | TYR | E | 150 | −17.463 | 61.805 | −15.227 | 1.00 | 90.25 |
| 7330 | CB | TYR | E | 150 | −17.811 | 60.622 | −16.175 | 1.00 | 141.76 |
| 7331 | CG | TYR | E | 150 | −19.266 | 60.196 | −16.334 | 1.00 | 141.76 |
| 7332 | CD1 | TYR | E | 150 | −20.183 | 60.995 | −17.006 | 1.00 | 141.76 |
| 7333 | CE1 | TYR | E | 150 | −21.503 | 60.584 | −17.180 | 1.00 | 141.76 |
| 7334 | CD2 | TYR | E | 150 | −19.711 | 58.965 | −15.839 | 1.00 | 141.76 |
| 7335 | CE2 | TYR | E | 150 | −21.030 | 58.545 | −16.010 | 1.00 | 141.76 |
| 7336 | CZ | TYR | E | 150 | −21.924 | 59.359 | −16.678 | 1.00 | 141.76 |
| 7337 | OH | TYR | E | 150 | −23.239 | 58.958 | −16.823 | 1.00 | 141.76 |
| 7338 | C | TYR | E | 150 | −18.481 | 62.934 | −15.293 | 1.00 | 90.25 |
| 7339 | O | TYR | E | 150 | −19.308 | 63.056 | −14.388 | 1.00 | 90.25 |
| 7340 | N | CYS | E | 151 | −18.404 | 63.792 | −16.309 | 1.00 | 89.54 |
| 7341 | CA | CYS | E | 151 | −19.360 | 64.895 | −16.421 | 1.00 | 89.54 |
| 7342 | C | CYS | E | 151 | −19.945 | 65.031 | −17.811 | 1.00 | 89.54 |
| 7343 | O | CYS | E | 151 | −19.354 | 64.573 | −18.778 | 1.00 | 89.54 |
| 7344 | CB | CYS | E | 151 | −18.713 | 66.218 | −16.017 | 1.00 | 116.04 |
| 7345 | SG | CYS | E | 151 | −17.189 | 66.664 | −16.901 | 1.00 | 116.04 |
| 7346 | N | THR | E | 152 | −21.118 | 65.650 | −17.904 | 1.00 | 145.06 |
| 7347 | CA | THR | E | 152 | −21.783 | 65.851 | −19.186 | 1.00 | 145.06 |
| 7348 | CB | THR | E | 152 | −23.138 | 65.100 | −19.247 | 1.00 | 246.19 |
| 7349 | OG1 | THR | E | 152 | −24.059 | 65.683 | −18.316 | 1.00 | 246.19 |
| 7350 | CG2 | THR | E | 152 | −22.944 | 63.634 | −18.897 | 1.00 | 246.19 |
| 7351 | C | THR | E | 152 | −22.032 | 67.345 | −19.332 | 1.00 | 145.06 |
| 7352 | O | THR | E | 152 | −22.255 | 68.039 | −18.337 | 1.00 | 145.06 |
| 7353 | N | GLY | E | 153 | −21.984 | 67.845 | −20.562 | 1.00 | 193.40 |
| 7354 | CA | GLY | E | 153 | −22.209 | 69.263 | −20.777 | 1.00 | 193.40 |
| 7355 | C | GLY | E | 153 | −22.478 | 69.578 | −22.227 | 1.00 | 193.40 |
| 7356 | O | GLY | E | 153 | −22.181 | 68.768 | −23.105 | 1.00 | 193.40 |
| 7357 | N | LYS | E | 154 | −23.044 | 70.751 | −22.486 | 1.00 | 120.13 |
| 7358 | CA | LYS | E | 154 | −23.331 | 71.135 | −23.857 | 1.00 | 120.13 |
| 7359 | CB | LYS | E | 154 | −24.722 | 71.763 | −23.950 | 1.00 | 168.61 |
| 7360 | CG | LYS | E | 154 | −25.193 | 72.073 | −25.366 | 1.00 | 168.61 |
| 7361 | CD | LYS | E | 154 | −26.607 | 72.634 | −25.316 | 1.00 | 168.61 |
| 7362 | CE | LYS | E | 154 | −27.120 | 73.053 | −26.682 | 1.00 | 168.61 |
| 7363 | NZ | LYS | E | 154 | −28.455 | 73.682 | −26.542 | 1.00 | 168.61 |
| 7364 | C | LYS | E | 154 | −22.271 | 72.122 | −24.309 | 1.00 | 120.13 |
| 7365 | O | LYS | E | 154 | −21.969 | 73.090 | −23.596 | 1.00 | 120.13 |
| 7366 | N | VAL | E | 155 | −21.685 | 71.856 | −25.475 | 1.00 | 169.05 |
| 7367 | CA | VAL | E | 155 | −20.666 | 72.731 | −26.041 | 1.00 | 169.05 |
| 7368 | CB | VAL | E | 155 | −19.362 | 71.985 | −26.310 | 1.00 | 148.26 |
| 7369 | CG1 | VAL | E | 155 | −18.328 | 72.927 | −26.910 | 1.00 | 148.26 |
| 7370 | CG2 | VAL | E | 155 | −18.847 | 71.420 | −25.025 | 1.00 | 148.26 |
| 7371 | C | VAL | E | 155 | −21.235 | 73.223 | −27.351 | 1.00 | 169.05 |
| 7372 | O | VAL | E | 155 | −21.684 | 72.425 | −28.174 | 1.00 | 169.05 |
| 7373 | N | TRP | E | 156 | −21.207 | 74.537 | −27.537 | 1.00 | 249.39 |
| 7374 | CA | TRP | E | 156 | −21.767 | 75.132 | −28.733 | 1.00 | 249.39 |
| 7375 | CB | TRP | E | 156 | −21.199 | 74.494 | −29.991 | 1.00 | 249.75 |
| 7376 | CG | TRP | E | 156 | −19.797 | 74.775 | −30.144 | 1.00 | 249.75 |
| 7377 | CD2 | TRP | E | 156 | −19.204 | 76.069 | −30.193 | 1.00 | 249.75 |
| 7378 | CE2 | TRP | E | 156 | −17.813 | 75.880 | −30.292 | 1.00 | 249.75 |
| 7379 | CE3 | TRP | E | 156 | −19.720 | 77.368 | −30.164 | 1.00 | 249.75 |
| 7380 | CD1 | TRP | E | 156 | −18.784 | 73.875 | −30.220 | 1.00 | 249.75 |
| 7381 | NE1 | TRP | E | 156 | −17.583 | 74.533 | −30.307 | 1.00 | 249.75 |
| 7382 | CZ2 | TRP | E | 156 | −16.919 | 76.947 | −30.375 | 1.00 | 249.75 |
| 7383 | CZ3 | TRP | E | 156 | −18.838 | 78.426 | −30.229 | 1.00 | 249.75 |
| 7384 | CH2 | TRP | E | 156 | −17.440 | 78.210 | −30.344 | 1.00 | 249.75 |
| 7385 | C | TRP | E | 156 | −23.225 | 74.814 | −28.688 | 1.00 | 249.39 |
| 7386 | O | TRP | E | 156 | −24.004 | 75.512 | −28.053 | 1.00 | 249.39 |
| 7387 | N | GLN | E | 157 | −23.571 | 73.711 | −29.338 | 1.00 | 249.35 |
| 7388 | CA | GLN | E | 157 | −24.945 | 73.299 | −29.404 | 1.00 | 249.35 |
| 7389 | CB | GLN | E | 157 | −25.559 | 73.927 | −30.645 | 1.00 | 249.42 |
| 7390 | CG | GLN | E | 157 | −25.723 | 75.415 | −30.458 | 1.00 | 249.42 |
| 7391 | CD | GLN | E | 157 | −26.491 | 75.691 | −29.186 | 1.00 | 249.42 |
| 7392 | OE1 | GLN | E | 157 | −27.538 | 75.108 | −28.977 | 1.00 | 249.42 |
| 7393 | NE2 | GLN | E | 157 | −25.980 | 76.575 | −28.340 | 1.00 | 249.42 |
| 7394 | C | GLN | E | 157 | −25.152 | 71.797 | −29.367 | 1.00 | 249.35 |
| 7395 | O | GLN | E | 157 | −26.238 | 71.302 | −29.670 | 1.00 | 249.35 |
| 7396 | N | LEU | E | 158 | −24.109 | 71.074 | −28.973 | 1.00 | 232.10 |
| 7397 | CA | LEU | E | 158 | −24.192 | 69.626 | −28.872 | 1.00 | 232.10 |
| 7398 | CB | LEU | E | 158 | −23.321 | 68.956 | −29.935 | 1.00 | 212.17 |
| 7399 | CG | LEU | E | 158 | −23.853 | 68.895 | −31.367 | 1.00 | 212.17 |
| 7400 | CD1 | LEU | E | 158 | −23.486 | 67.531 | −31.928 | 1.00 | 212.17 |
| 7401 | CD2 | LEU | E | 158 | −25.370 | 69.079 | −31.415 | 1.00 | 212.17 |
| 7402 | C | LEU | E | 158 | −23.788 | 69.124 | −27.492 | 1.00 | 232.10 |
| 7403 | O | LEU | E | 158 | −23.007 | 69.767 | −26.784 | 1.00 | 232.10 |
| 7404 | N | ASP | E | 159 | −24.333 | 67.969 | −27.122 | 1.00 | 245.44 |
| 7405 | CA | ASP | E | 159 | −24.058 | 67.353 | −25.830 | 1.00 | 245.44 |
| 7406 | CB | ASP | E | 159 | −25.270 | 66.525 | −25.381 | 1.00 | 211.53 |
| 7407 | CG | ASP | E | 159 | −26.567 | 67.329 | −25.373 | 1.00 | 211.53 |
| 7408 | OD1 | ASP | E | 159 | −26.659 | 68.326 | −24.626 | 1.00 | 211.53 |
| 7409 | OD2 | ASP | E | 159 | −27.502 | 66.961 | −26.118 | 1.00 | 211.53 |
| 7410 | C | ASP | E | 159 | −22.822 | 66.454 | −25.908 | 1.00 | 245.44 |
| 7411 | O | ASP | E | 159 | −22.578 | 65.818 | −26.936 | 1.00 | 245.44 |
| 7412 | N | TYR | E | 160 | −22.046 | 66.411 | −24.824 | 1.00 | 168.54 |
| 7413 | CA | TYR | E | 160 | −20.842 | 65.579 | −24.759 | 1.00 | 168.54 |
| 7414 | CB | TYR | E | 160 | −19.592 | 66.368 | −25.140 | 1.00 | 216.78 |
| 7415 | CG | TYR | E | 160 | −19.684 | 67.078 | −26.461 | 1.00 | 216.78 |
| 7416 | CD1 | TYR | E | 160 | −20.168 | 68.381 | −26.533 | 1.00 | 216.78 |
| 7417 | CE1 | TYR | E | 160 | −20.270 | 69.041 | −27.747 | 1.00 | 216.78 |
| 7418 | CD2 | TYR | E | 160 | −19.301 | 66.446 | −27.644 | 1.00 | 216.78 |
| 7419 | CE2 | TYR | E | 160 | −19.401 | 67.097 | −28.868 | 1.00 | 216.78 |
| 7420 | CZ | TYR | E | 160 | −19.887 | 68.395 | −28.909 | 1.00 | 216.78 |
| 7421 | OH | TYR | E | 160 | −20.003 | 69.045 | −30.114 | 1.00 | 216.78 |
| 7422 | C | TYR | E | 160 | −20.609 | 64.973 | −23.383 | 1.00 | 168.54 |
| 7423 | O | TYR | E | 160 | −20.898 | 65.581 | −22.351 | 1.00 | 168.54 |
| 7424 | N | GLU | E | 161 | −20.048 | 63.771 | −23.399 | 1.00 | 118.65 |
| 7425 | CA | GLU | E | 161 | −19.738 | 63.004 | −22.200 | 1.00 | 118.65 |
| 7426 | CB | GLU | E | 161 | −20.378 | 61.624 | −22.330 | 1.00 | 174.81 |
| 7427 | CG | GLU | E | 161 | −20.107 | 60.665 | −21.205 | 1.00 | 174.81 |
| 7428 | CD | GLU | E | 161 | −21.068 | 59.496 | −21.241 | 1.00 | 174.81 |
| 7429 | OE1 | GLU | E | 161 | −20.765 | 58.447 | −20.632 | 1.00 | 174.81 |
| 7430 | OE2 | GLU | E | 161 | −22.138 | 59.638 | −21.876 | 1.00 | 174.81 |
| 7431 | C | GLU | E | 161 | −18.214 | 62.890 | −22.087 | 1.00 | 118.65 |
| 7432 | O | GLU | E | 161 | −17.529 | 62.655 | −23.085 | 1.00 | 118.65 |
| 7433 | N | SER | E | 162 | −17.688 | 63.066 | −20.877 | 1.00 | 111.13 |
| 7434 | CA | SER | E | 162 | −16.249 | 63.005 | −20.643 | 1.00 | 111.13 |
| 7435 | CB | SER | E | 162 | −15.864 | 63.955 | −19.512 | 1.00 | 104.26 |
| 7436 | OG | SER | E | 162 | −16.548 | 63.616 | −18.315 | 1.00 | 104.26 |
| 7437 | C | SER | E | 162 | −15.790 | 61.603 | −20.296 | 1.00 | 111.13 |
| 7438 | O | SER | E | 162 | −16.609 | 60.725 | −20.057 | 1.00 | 111.13 |
| 7439 | N | GLU | E | 163 | −14.478 | 61.391 | −20.272 | 1.00 | 89.90 |
| 7440 | CA | GLU | E | 163 | −13.915 | 60.079 | −19.943 | 1.00 | 89.90 |
| 7441 | CB | GLU | E | 163 | −12.417 | 60.050 | −20.253 | 1.00 | 240.41 |
| 7442 | CG | GLU | E | 163 | −12.072 | 59.972 | −21.731 | 1.00 | 240.41 |
| 7443 | CD | GLU | E | 163 | −12.359 | 58.606 | −22.321 | 1.00 | 240.41 |
| 7444 | OE1 | GLU | E | 163 | −11.812 | 57.609 | −21.802 | 1.00 | 240.41 |
| 7445 | OE2 | GLU | E | 163 | −13.128 | 58.527 | −23.303 | 1.00 | 240.41 |
| 7446 | C | GLU | E | 163 | −14.131 | 59.856 | −18.455 | 1.00 | 89.90 |
| 7447 | O | GLU | E | 163 | −14.028 | 60.795 | −17.668 | 1.00 | 89.90 |
| 7448 | N | PRO | E | 164 | −14.433 | 58.609 | −18.041 | 1.00 | 64.59 |
| 7449 | CD | PRO | E | 164 | −14.576 | 57.360 | −18.788 | 1.00 | 100.06 |
| 7450 | CA | PRO | E | 164 | −14.639 | 58.395 | −16.606 | 1.00 | 64.59 |
| 7451 | CB | PRO | E | 164 | −15.248 | 57.008 | −16.568 | 1.00 | 100.06 |
| 7452 | CG | PRO | E | 164 | −14.564 | 56.333 | −17.682 | 1.00 | 100.06 |
| 7453 | C | PRO | E | 164 | −13.327 | 58.488 | −15.846 | 1.00 | 64.59 |
| 7454 | O | PRO | E | 164 | −12.243 | 58.317 | −16.415 | 1.00 | 64.59 |
| 7455 | N | LEU | E | 165 | −13.405 | 58.777 | −14.554 | 1.00 | 77.47 |
| 7456 | CA | LEU | E | 165 | −12.197 | 58.901 | −13.758 | 1.00 | 77.47 |
| 7457 | CB | LEU | E | 165 | −11.771 | 60.364 | −13.685 | 1.00 | 63.52 |
| 7458 | CG | LEU | E | 165 | −10.647 | 60.607 | −12.688 | 1.00 | 63.52 |
| 7459 | CD1 | LEU | E | 165 | −9.568 | 59.589 | −12.978 | 1.00 | 63.52 |
| 7460 | CD2 | LEU | E | 165 | −10.088 | 61.981 | −12.800 | 1.00 | 63.52 |
| 7461 | C | LEU | E | 165 | −12.415 | 58.362 | −12.360 | 1.00 | 77.47 |
| 7462 | O | LEU | E | 165 | −13.328 | 58.803 | −11.665 | 1.00 | 77.47 |
| 7463 | N | ASN | E | 166 | −11.580 | 57.407 | −11.959 | 1.00 | 93.16 |
| 7464 | CA | ASN | E | 166 | −11.684 | 56.801 | −10.635 | 1.00 | 93.16 |
| 7465 | CB | ASN | E | 166 | −11.050 | 55.421 | −10.614 | 1.00 | 96.38 |
| 7466 | CG | ASN | E | 166 | −12.037 | 54.314 | −10.900 | 1.00 | 96.38 |
| 7467 | OD1 | ASN | E | 166 | −13.220 | 54.408 | −10.581 | 1.00 | 96.38 |
| 7468 | ND2 | ASN | E | 166 | −11.534 | 53.242 | −11.486 | 1.00 | 96.38 |
| 7469 | C | ASN | E | 166 | −10.999 | 57.633 | −9.582 | 1.00 | 93.16 |
| 7470 | O | ASN | E | 166 | −9.975 | 58.232 | −9.837 | 1.00 | 93.16 |
| 7471 | N | ILE | E | 167 | −11.544 | 57.639 | −8.380 | 1.00 | 64.50 |
| 7472 | CA | ILE | E | 167 | −10.971 | 58.424 | −7.303 | 1.00 | 64.50 |
| 7473 | CB | ILE | E | 167 | −11.751 | 59.709 | −7.114 | 1.00 | 67.49 |
| 7474 | CG2 | ILE | E | 167 | −11.452 | 60.314 | −5.762 | 1.00 | 67.49 |
| 7475 | CG1 | ILE | E | 167 | −11.427 | 60.672 | −8.243 | 1.00 | 67.49 |
| 7476 | CD1 | ILE | E | 167 | −12.043 | 62.011 | −8.046 | 1.00 | 67.49 |
| 7477 | C | ILE | E | 167 | −11.054 | 57.651 | −6.012 | 1.00 | 64.50 |
| 7478 | O | ILE | E | 167 | −12.117 | 57.081 | −5.675 | 1.00 | 64.50 |
| 7479 | N | THR | E | 188 | −9.963 | 57.632 | −5.266 | 1.00 | 85.23 |
| 7480 | CA | THR | E | 168 | −9.997 | 56.899 | −4.025 | 1.00 | 85.23 |
| 7481 | CB | THR | E | 168 | −9.077 | 55.692 | −4.075 | 1.00 | 118.46 |
| 7482 | OG1 | THR | E | 168 | −9.470 | 54.848 | −5.162 | 1.00 | 118.46 |
| 7483 | CG2 | THR | E | 168 | −9.178 | 54.910 | −2.787 | 1.00 | 118.46 |
| 7484 | C | THR | E | 168 | −9.621 | 57.759 | −2.856 | 1.00 | 85.23 |
| 7485 | O | THR | E | 168 | −8.681 | 58.558 | −2.931 | 1.00 | 85.23 |
| 7486 | N | VAL | E | 169 | −10.379 | 57.600 | −1.781 | 1.00 | 97.28 |
| 7487 | CA | VAL | E | 169 | −10.150 | 58.320 | −0.544 | 1.00 | 97.28 |
| 7488 | CB | VAL | E | 169 | −11.420 | 59.087 | −0.122 | 1.00 | 79.18 |
| 7489 | CG1 | VAL | E | 169 | −11.346 | 59.434 | 1.333 | 1.00 | 79.18 |
| 7490 | CG2 | VAL | E | 169 | −11.565 | 60.340 | −0.927 | 1.00 | 79.18 |
| 7491 | C | VAL | E | 169 | −9.809 | 57.241 | 0.489 | 1.00 | 97.28 |
| 7492 | O | VAL | E | 169 | −10.681 | 56.475 | 0.905 | 1.00 | 97.28 |
| 7493 | N | ILE | E | 170 | −8.538 | 57.156 | 0.876 | 1.00 | 87.96 |
| 7494 | CA | ILE | E | 170 | −8.113 | 56.159 | 1.856 | 1.00 | 87.96 |
| 7495 | CB | ILE | E | 170 | −6.663 | 55.682 | 1.574 | 1.00 | 99.84 |
| 7496 | CG2 | ILE | E | 170 | −6.530 | 55.259 | 0.124 | 1.00 | 99.84 |
| 7497 | CG1 | ILE | E | 170 | −5.666 | 56.808 | 1.822 | 1.00 | 99.84 |
| 7498 | CD1 | ILE | E | 170 | −4.217 | 56.427 | 1.528 | 1.00 | 99.84 |
| 7499 | C | ILE | E | 170 | −8.209 | 56.759 | 3.253 | 1.00 | 87.96 |
| 7500 | O | ILE | E | 170 | −8.544 | 57.933 | 3.392 | 1.00 | 87.96 |
| 7501 | N | LYS | E | 171 | −7.932 | 55.970 | 4.286 | 1.00 | 171.73 |
| 7502 | CA | LYS | E | 171 | −8.001 | 56.486 | 5.650 | 1.00 | 171.73 |
| 7503 | CB | LYS | E | 171 | −9.242 | 55.933 | 6.353 | 1.00 | 217.19 |
| 7504 | CG | LYS | E | 171 | −9.308 | 54.421 | 6.380 | 1.00 | 217.19 |
| 7505 | CD | LYS | E | 171 | −10.739 | 53.932 | 6.537 | 1.00 | 217.19 |
| 7506 | CE | LYS | E | 171 | −11.376 | 54.437 | 7.821 | 1.00 | 217.19 |
| 7507 | NZ | LYS | E | 171 | −12.786 | 53.971 | 7.947 | 1.00 | 217.19 |
| 7508 | C | LYS | E | 171 | −6.749 | 56.162 | 6.454 | 1.00 | 171.73 |
| 7509 | O | LYS | E | 171 | −6.573 | 56.658 | 7.565 | 1.00 | 171.73 |
| 7510 | C1 | NAG | E | 221 | 0.947 | 78.578 | −23.161 | 1.00 | 249.29 |
| 7511 | C2 | NAG | E | 221 | −0.412 | 79.265 | −23.224 | 1.00 | 249.29 |
| 7512 | N2 | NAG | E | 221 | −1.456 | 78.261 | −23.255 | 1.00 | 249.29 |
| 7513 | C7 | NAG | E | 221 | −2.671 | 78.553 | −22.807 | 1.00 | 249.29 |
| 7514 | O7 | NAG | E | 221 | −2.963 | 79.651 | −22.339 | 1.00 | 249.29 |
| 7515 | C8 | NAG | E | 221 | −3.720 | 77.456 | −22.880 | 1.00 | 249.29 |
| 7516 | C3 | NAG | E | 221 | −0.518 | 80.128 | −24.473 | 1.00 | 249.29 |
| 7517 | O3 | NAG | E | 221 | −1.714 | 80.890 | −24.425 | 1.00 | 249.29 |
| 7518 | C4 | NAG | E | 221 | 0.670 | 81.073 | −24.631 | 1.00 | 249.29 |
| 7519 | O4 | NAG | E | 221 | 0.579 | 81.653 | −25.947 | 1.00 | 249.29 |
| 7520 | C5 | NAG | E | 221 | 1.997 | 80.296 | −24.470 | 1.00 | 249.29 |
| 7521 | O5 | NAG | E | 221 | 1.994 | 79.555 | −23.228 | 1.00 | 249.29 |
| 7522 | C6 | NAG | E | 221 | 3.222 | 81.198 | −24.429 | 1.00 | 249.29 |
| 7523 | O6 | NAG | E | 221 | 3.160 | 82.105 | −23.335 | 1.00 | 249.29 |
| 7524 | C1 | NAG | E | 222 | 1.316 | 82.790 | −26.227 | 1.00 | 249.77 |
| 7525 | C2 | NAG | E | 222 | 0.449 | 83.797 | −27.008 | 1.00 | 249.77 |
| 7526 | N2 | NAG | E | 222 | −0.713 | 84.171 | −26.221 | 1.00 | 249.77 |
| 7527 | C7 | NAG | E | 222 | −0.903 | 85.441 | −25.867 | 1.00 | 249.77 |
| 7528 | O7 | NAG | E | 222 | −0.130 | 86.350 | −26.178 | 1.00 | 249.77 |
| 7529 | C8 | NAG | E | 222 | −2.140 | 85.750 | −25.043 | 1.00 | 249.77 |
| 7530 | C3 | NAG | E | 222 | 0.003 | 83.194 | −28.351 | 1.00 | 249.77 |
| 7531 | O3 | NAG | E | 222 | −0.664 | 84.182 | −29.124 | 1.00 | 249.77 |
| 7532 | C4 | NAG | E | 222 | 1.211 | 82.656 | −29.133 | 1.00 | 249.77 |
| 7533 | O4 | NAG | E | 222 | 0.762 | 81.952 | −30.285 | 1.00 | 249.77 |
| 7534 | C5 | NAG | E | 222 | 2.048 | 81.716 | −28.248 | 1.00 | 249.77 |
| 7535 | O5 | NAG | E | 222 | 2.440 | 82.386 | −27.023 | 1.00 | 249.77 |
| 7536 | C6 | NAG | E | 222 | 3.319 | 81.240 | −28.926 | 1.00 | 249.77 |
| 7537 | O6 | NAG | E | 222 | 3.494 | 79.843 | −28.749 | 1.00 | 249.77 |
| 7538 | C1 | NAG | E | 242 | 6.691 | 58.325 | −21.511 | 1.00 | 184.18 |
| 7539 | C2 | NAG | E | 242 | 6.772 | 58.888 | −22.927 | 1.00 | 184.18 |
| 7540 | N2 | NAG | E | 242 | 7.616 | 60.057 | −22.949 | 1.00 | 184.18 |
| 7541 | C7 | NAG | E | 242 | 8.669 | 60.081 | −23.755 | 1.00 | 184.18 |
| 7542 | O7 | NAG | E | 242 | 8.972 | 59.137 | −24.489 | 1.00 | 184.18 |
| 7543 | C8 | NAG | E | 242 | 9.523 | 61.338 | −23.746 | 1.00 | 184.18 |
| 7544 | C3 | NAG | E | 242 | 5.382 | 59.264 | −23.429 | 1.00 | 184.18 |
| 7545 | O3 | NAG | E | 242 | 5.460 | 59.693 | −24.778 | 1.00 | 184.18 |
| 7546 | C4 | NAG | E | 242 | 4.452 | 58.056 | −23.332 | 1.00 | 184.18 |
| 7547 | O4 | NAG | E | 242 | 3.102 | 58.481 | −23.616 | 1.00 | 184.18 |
| 7548 | C5 | NAG | E | 242 | 4.513 | 57.446 | −21.911 | 1.00 | 184.18 |
| 7549 | O5 | NAG | E | 242 | 5.874 | 57.166 | −21.520 | 1.00 | 184.18 |
| 7550 | C6 | NAG | E | 242 | 3.835 | 56.114 | −21.900 | 1.00 | 184.18 |
| 7551 | O6 | NAG | E | 242 | 2.768 | 56.046 | −20.979 | 1.00 | 184.18 |
| 7552 | C1 | NAG | E | 243 | 2.525 | 57.919 | −24.745 | 1.00 | 162.87 |
| 7553 | C2 | NAG | E | 243 | 0.990 | 57.891 | −24.616 | 1.00 | 162.87 |
| 7554 | N2 | NAG | E | 243 | 0.580 | 57.065 | −23.493 | 1.00 | 162.87 |
| 7555 | C7 | NAG | E | 243 | −0.334 | 57.510 | −22.639 | 1.00 | 162.87 |
| 7556 | O7 | NAG | E | 243 | −0.848 | 58.623 | −22.738 | 1.00 | 162.87 |
| 7557 | C8 | NAG | E | 243 | −0.729 | 56.592 | −21.497 | 1.00 | 162.87 |
| 7558 | C3 | NAG | E | 243 | 0.393 | 57.321 | −25.904 | 1.00 | 162.87 |
| 7559 | O3 | NAG | E | 243 | −1.018 | 57.363 | −25.842 | 1.00 | 162.87 |
| 7560 | C4 | NAG | E | 243 | 0.891 | 58.108 | −27.133 | 1.00 | 162.87 |
| 7561 | O4 | NAG | E | 243 | 0.428 | 57.479 | −28.366 | 1.00 | 162.87 |
| 7562 | C5 | NAG | E | 243 | 2.430 | 58.133 | −27.118 | 1.00 | 162.87 |
| 7563 | O5 | NAG | E | 243 | 2.904 | 58.707 | −25.885 | 1.00 | 162.87 |
| 7564 | C6 | NAG | E | 243 | 3.044 | 58.927 | −28.250 | 1.00 | 162.87 |
| 7565 | O6 | NAG | E | 243 | 2.770 | 60.311 | −28.097 | 1.00 | 162.87 |
| 7566 | C1 | MAN | E | 244 | −0.169 | 58.185 | −29.362 | 1.00 | 177.48 |
| 7567 | C2 | MAN | E | 244 | −1.467 | 58.963 | −29.047 | 1.00 | 177.48 |
| 7568 | O2 | MAN | E | 244 | −1.159 | 60.326 | −28.837 | 1.00 | 177.48 |
| 7569 | C3 | MAN | E | 244 | −2.273 | 58.794 | −30.382 | 1.00 | 177.48 |
| 7570 | O3 | MAN | E | 244 | −3.531 | 59.444 | −30.342 | 1.00 | 177.48 |
| 7571 | C4 | MAN | E | 244 | −1.469 | 59.230 | −31.646 | 1.00 | 177.48 |
| 7572 | O4 | MAN | E | 244 | −2.267 | 59.074 | −32.823 | 1.00 | 177.48 |
| 7573 | C5 | MAN | E | 244 | −0.223 | 58.317 | −31.725 | 1.00 | 177.48 |
| 7574 | O5 | MAN | E | 244 | 0.620 | 58.472 | −30.547 | 1.00 | 177.48 |
| 7575 | C6 | MAN | E | 244 | 0.611 | 58.487 | −33.000 | 1.00 | 177.48 |
| 7576 | O6 | MAN | E | 244 | 1.488 | 59.592 | −32.913 | 1.00 | 177.48 |
| 7577 | C1 | NAG | E | 250 | 13.381 | 78.909 | −13.725 | 1.00 | 249.71 |
| 7578 | C2 | NAG | E | 250 | 12.909 | 80.209 | −13.049 | 1.00 | 249.71 |
| 7579 | N2 | NAG | E | 250 | 13.077 | 80.124 | −11.608 | 1.00 | 249.71 |
| 7580 | C7 | NAG | E | 250 | 13.987 | 80.876 | −10.993 | 1.00 | 249.71 |
| 7581 | O7 | NAG | E | 250 | 14.727 | 81.658 | −11.592 | 1.00 | 249.71 |
| 7582 | C8 | NAG | E | 250 | 14.097 | 80.733 | −9.481 | 1.00 | 249.71 |
| 7583 | C3 | NAG | E | 250 | 11.429 | 80.446 | −13.387 | 1.00 | 249.71 |
| 7584 | O3 | NAG | E | 250 | 11.000 | 81.693 | −12.858 | 1.00 | 249.71 |
| 7585 | C4 | NAG | E | 250 | 11.216 | 80.427 | −14.906 | 1.00 | 249.71 |
| 7586 | O4 | NAG | E | 250 | 9.826 | 80.512 | −15.194 | 1.00 | 249.71 |
| 7587 | C5 | NAG | E | 250 | 11.793 | 79.133 | −15.504 | 1.00 | 249.71 |
| 7588 | O5 | NAG | E | 250 | 13.187 | 78.993 | −15.143 | 1.00 | 249.71 |
| 7589 | C6 | NAG | E | 250 | 11.720 | 79.107 | −17.018 | 1.00 | 249.71 |
| 7590 | O6 | NAG | E | 250 | 12.531 | 78.071 | −17.553 | 1.00 | 249.71 |
| 7591 | C1 | NAG | E | 274 | 17.952 | 58.017 | 0.947 | 1.00 | 232.95 |
| 7592 | C2 | NAG | E | 274 | 17.034 | 57.505 | 2.065 | 1.00 | 232.95 |
| 7593 | N2 | NAG | E | 274 | 16.704 | 58.587 | 2.972 | 1.00 | 232.95 |
| 7594 | C7 | NAG | E | 274 | 15.587 | 58.533 | 3.690 | 1.00 | 232.95 |
| 7595 | O7 | NAG | E | 274 | 14.789 | 57.594 | 3.617 | 1.00 | 232.95 |
| 7596 | C8 | NAG | E | 274 | 15.307 | 59.699 | 4.627 | 1.00 | 232.95 |
| 7597 | C3 | NAG | E | 274 | 17.729 | 56.379 | 2.842 | 1.00 | 232.95 |
| 7598 | O3 | NAG | E | 274 | 16.822 | 55.816 | 3.780 | 1.00 | 232.95 |
| 7599 | C4 | NAG | E | 274 | 18.227 | 55.288 | 1.888 | 1.00 | 232.95 |
| 7600 | O4 | NAG | E | 274 | 18.999 | 54.339 | 2.613 | 1.00 | 232.95 |
| 7601 | C5 | NAG | E | 274 | 19.081 | 55.909 | 0.772 | 1.00 | 232.95 |
| 7602 | O5 | NAG | E | 274 | 18.329 | 56.933 | 0.083 | 1.00 | 232.95 |
| 7603 | C6 | NAG | E | 274 | 19.520 | 54.898 | −0.274 | 1.00 | 232.95 |
| 7604 | O6 | NAG | E | 274 | 20.106 | 55.536 | −1.399 | 1.00 | 232.95 |
| 7605 | C1 | NAG | E | 335 | −12.841 | 75.891 | −12.527 | 1.00 | 244.27 |
| 7606 | C2 | NAG | E | 335 | −11.869 | 76.721 | −11.656 | 1.00 | 244.27 |
| 7607 | N2 | NAG | E | 335 | −12.291 | 76.605 | −10.271 | 1.00 | 244.27 |
| 7608 | C7 | NAG | E | 335 | −11.503 | 76.035 | −9.365 | 1.00 | 244.27 |
| 7609 | O7 | NAG | E | 335 | −10.386 | 75.589 | −9.628 | 1.00 | 244.27 |
| 7610 | C8 | NAG | E | 335 | −12.039 | 75.956 | −7.947 | 1.00 | 244.27 |
| 7611 | C3 | NAG | E | 335 | −11.803 | 78.214 | −12.025 | 1.00 | 244.27 |
| 7612 | O3 | NAG | E | 335 | −10.618 | 78.779 | −11.480 | 1.00 | 244.27 |
| 7613 | C4 | NAG | E | 335 | −11.806 | 78.418 | −13.537 | 1.00 | 244.27 |
| 7614 | O4 | NAG | E | 335 | −11.818 | 79.805 | −13.844 | 1.00 | 244.27 |
| 7615 | C5 | NAG | E | 335 | −13.044 | 77.739 | −14.108 | 1.00 | 244.27 |
| 7616 | O5 | NAG | E | 335 | −12.940 | 76.310 | −13.913 | 1.00 | 244.27 |
| 7617 | C6 | NAG | E | 335 | −13.184 | 77.982 | −15.605 | 1.00 | 244.27 |
| 7618 | O6 | NAG | E | 335 | −14.397 | 78.652 | −15.913 | 1.00 | 244.27 |
| 7619 | C1 | NAG | E | 340 | −14.368 | 66.477 | 8.751 | 1.00 | 249.77 |
| 7620 | C2 | NAG | E | 340 | −13.779 | 65.349 | 9.574 | 1.00 | 249.77 |
| 7621 | N2 | NAG | E | 340 | −13.415 | 64.233 | 8.721 | 1.00 | 249.77 |
| 7622 | C7 | NAG | E | 340 | −12.195 | 63.711 | 8.818 | 1.00 | 249.77 |
| 7623 | O7 | NAG | E | 340 | −11.344 | 64.132 | 9.612 | 1.00 | 249.77 |
| 7624 | C8 | NAG | E | 340 | −11.863 | 62.550 | 7.900 | 1.00 | 249.77 |
| 7625 | C3 | NAG | E | 340 | −14.783 | 64.920 | 10.636 | 1.00 | 249.77 |
| 7626 | O3 | NAG | E | 340 | −14.195 | 63.909 | 11.453 | 1.00 | 249.77 |
| 7627 | C4 | NAG | E | 340 | −15.166 | 66.132 | 11.500 | 1.00 | 249.77 |
| 7628 | O4 | NAG | E | 340 | −16.238 | 65.759 | 12.355 | 1.00 | 249.77 |
| 7629 | C5 | NAG | E | 340 | −15.575 | 67.356 | 10.636 | 1.00 | 249.77 |
| 7630 | O5 | NAG | E | 340 | −14.610 | 67.605 | 9.591 | 1.00 | 249.77 |
| 7631 | C6 | NAG | E | 340 | −15.666 | 68.648 | 11.433 | 1.00 | 249.77 |
| 7632 | O6 | NAG | E | 340 | −15.300 | 69.781 | 10.659 | 1.00 | 249.77 |
| 7633 | C1 | NAG | E | 366 | −12.398 | 52.150 | −11.858 | 1.00 | 131.22 |
| 7634 | C2 | NAG | E | 366 | −11.828 | 51.489 | −13.095 | 1.00 | 131.22 |
| 7635 | N2 | NAG | E | 366 | −11.760 | 52.463 | −14.162 | 1.00 | 131.22 |
| 7636 | C7 | NAG | E | 366 | −10.652 | 53.170 | −14.339 | 1.00 | 131.22 |
| 7637 | O7 | NAG | E | 366 | −9.658 | 53.028 | −13.631 | 1.00 | 131.22 |
| 7638 | C8 | NAG | E | 366 | −10.642 | 54.189 | −15.474 | 1.00 | 131.22 |
| 7639 | C3 | NAG | E | 366 | −12.712 | 50.337 | −13.517 | 1.00 | 131.22 |
| 7640 | O3 | NAG | E | 366 | −12.088 | 49.646 | −14.588 | 1.00 | 131.22 |
| 7641 | C4 | NAG | E | 366 | −12.958 | 49.373 | −12.351 | 1.00 | 131.22 |
| 7642 | O4 | NAG | E | 366 | −13.982 | 48.430 | −12.735 | 1.00 | 131.22 |
| 7643 | C5 | NAG | E | 366 | −13.414 | 50.137 | −11.096 | 1.00 | 131.22 |
| 7644 | O5 | NAG | E | 366 | −12.496 | 51.204 | −10.795 | 1.00 | 131.22 |
| 7645 | C6 | NAG | E | 366 | −13.478 | 49.261 | −9.862 | 1.00 | 131.22 |
| 7646 | O6 | NAG | E | 366 | −13.939 | 49.998 | −8.740 | 1.00 | 131.22 |
| 7647 | C1 | NAG | E | 367 | −13.682 | 47.077 | −12.614 | 1.00 | 245.35 |
| 7648 | C2 | NAG | E | 367 | −14.975 | 46.261 | −12.520 | 1.00 | 245.35 |
| 7649 | N2 | NAG | E | 367 | −15.776 | 46.701 | −11.394 | 1.00 | 245.35 |
| 7650 | C7 | NAG | E | 367 | −16.904 | 47.372 | −11.610 | 1.00 | 245.35 |
| 7651 | O7 | NAG | E | 367 | −17.315 | 47.646 | −12.739 | 1.00 | 245.35 |
| 7652 | C8 | NAG | E | 367 | −17.698 | 47.808 | −10.389 | 1.00 | 245.35 |
| 7653 | C3 | NAG | E | 367 | −14.620 | 44.778 | −12.391 | 1.00 | 245.35 |
| 7654 | O3 | NAG | E | 367 | −15.804 | 43.995 | −12.351 | 1.00 | 245.35 |
| 7655 | C4 | NAG | E | 367 | −13.757 | 44.354 | −13.584 | 1.00 | 245.35 |
| 7656 | O4 | NAG | E | 367 | −13.340 | 43.005 | −13.423 | 1.00 | 245.35 |
| 7657 | C5 | NAG | E | 367 | −12.529 | 45.270 | −13.701 | 1.00 | 245.35 |
| 7658 | O5 | NAG | E | 367 | −12.935 | 46.662 | −13.772 | 1.00 | 245.35 |
| 7659 | C6 | NAG | E | 367 | −11.710 | 44.973 | −14.941 | 1.00 | 245.35 |
| 7660 | O6 | NAG | E | 367 | −11.792 | 46.031 | −15.884 | 1.00 | 245.35 |

TABLE 6
Atomic coordinates of PhFcεRIα₁₋₁₇₂, Form T2

| ATOM | ATOM | | | | | | | | |
|---|---|---|---|---|---|---|---|---|---|
| NUMBER | TYPE | RESIDUE | | # | X | Y | Z | occ | B |
| 1 | CB | LYS | C | 4 | 16.063 | 45.227 | 50.293 | 1.00 | 240.56 |
| 2 | CG | LYS | C | 4 | 17.178 | 44.372 | 49.692 | 1.00 | 240.56 |
| 3 | CD | LYS | C | 4 | 18.081 | 43.766 | 50.766 | 1.00 | 240.56 |
| 4 | CE | LYS | C | 4 | 19.152 | 42.864 | 50.151 | 1.00 | 240.56 |
| 5 | NZ | LYS | C | 4 | 20.054 | 42.261 | 51.173 | 1.00 | 240.56 |
| 6 | C | LYS | C | 4 | 14.440 | 44.631 | 48.479 | 1.00 | 248.46 |
| 7 | O | LYS | C | 4 | 14.364 | 43.506 | 48.972 | 1.00 | 248.46 |
| 8 | N | LYS | C | 4 | 14.039 | 46.614 | 49.935 | 1.00 | 248.46 |
| 9 | CA | LYS | C | 4 | 15.077 | 45.783 | 49.257 | 1.00 | 248.46 |
| 10 | N | PRO | C | 5 | 13.962 | 44.902 | 47.256 | 1.00 | 240.49 |
| 11 | CD | PRO | C | 5 | 13.761 | 46.229 | 46.635 | 1.00 | 226.60 |
| 12 | CA | PRO | C | 5 | 13.338 | 43.853 | 46.448 | 1.00 | 240.49 |
| 13 | CB | PRO | C | 5 | 12.401 | 44.636 | 45.543 | 1.00 | 226.60 |
| 14 | CG | PRO | C | 5 | 13.189 | 45.881 | 45.274 | 1.00 | 226.60 |
| 15 | C | PRO | C | 5 | 14.379 | 43.053 | 45.660 | 1.00 | 240.49 |
| 16 | O | PRO | C | 5 | 15.487 | 43.534 | 45.409 | 1.00 | 240.49 |
| 17 | N | LYS | C | 6 | 14.022 | 41.831 | 45.280 | 1.00 | 200.38 |
| 18 | CA | LYS | C | 6 | 14.932 | 40.986 | 44.518 | 1.00 | 200.38 |
| 19 | CB | LYS | C | 6 | 15.670 | 40.017 | 45.446 | 1.00 | 249.33 |
| 20 | CG | LYS | C | 6 | 16.701 | 39.153 | 44.729 | 1.00 | 249.33 |
| 21 | CD | LYS | C | 6 | 17.530 | 38.312 | 45.692 | 1.00 | 249.33 |
| 22 | CE | LYS | C | 6 | 18.564 | 37.480 | 44.943 | 1.00 | 249.33 |
| 23 | NZ | LYS | C | 6 | 19.471 | 36.732 | 45.855 | 1.00 | 249.33 |
| 24 | C | LYS | C | 6 | 14.168 | 40.207 | 43.449 | 1.00 | 200.38 |
| 25 | O | LYS | C | 6 | 13.352 | 39.327 | 43.755 | 1.00 | 200.38 |
| 26 | N | VAL | C | 7 | 14.451 | 40.538 | 42.190 | 1.00 | 184.84 |
| 27 | CA | VAL | C | 7 | 13.799 | 39.902 | 41.052 | 1.00 | 184.84 |
| 28 | CB | VAL | C | 7 | 14.155 | 40.623 | 39.744 | 1.00 | 175.84 |
| 29 | CG1 | VAL | C | 7 | 13.207 | 40.181 | 38.645 | 1.00 | 175.84 |
| 30 | CG2 | VAL | C | 7 | 14.108 | 42.134 | 39.944 | 1.00 | 175.84 |
| 31 | C | VAL | C | 7 | 14.153 | 38.431 | 40.884 | 1.00 | 184.84 |
| 32 | O | VAL | C | 7 | 15.316 | 38.073 | 40.746 | 1.00 | 184.84 |
| 33 | N | SER | C | 8 | 13.132 | 37.584 | 40.887 | 1.00 | 212.94 |
| 34 | CA | SER | C | 8 | 13.318 | 36.148 | 40.720 | 1.00 | 212.94 |
| 35 | CB | SER | C | 8 | 12.487 | 35.385 | 41.758 | 1.00 | 203.15 |
| 36 | OG | SER | C | 8 | 11.148 | 35.858 | 41.801 | 1.00 | 203.15 |
| 37 | C | SER | C | 8 | 12.886 | 35.755 | 39.307 | 1.00 | 212.94 |
| 38 | O | SER | C | 8 | 12.169 | 36.508 | 38.646 | 1.00 | 212.94 |
| 39 | N | LEU | C | 9 | 13.330 | 34.593 | 38.834 | 1.00 | 249.13 |
| 40 | CA | LEU | C | 9 | 12.955 | 34.137 | 37.495 | 1.00 | 249.13 |
| 41 | CB | LEU | C | 9 | 14.150 | 34.163 | 36.540 | 1.00 | 143.92 |
| 42 | CG | LEU | C | 9 | 14.916 | 35.465 | 36.269 | 1.00 | 143.92 |
| 43 | CD1 | LEU | C | 9 | 15.771 | 35.258 | 35.022 | 1.00 | 143.92 |
| 44 | CD2 | LEU | C | 9 | 13.966 | 36.637 | 36.063 | 1.00 | 143.92 |
| 45 | C | LEU | C | 9 | 12.395 | 32.728 | 37.507 | 1.00 | 249.13 |
| 46 | O | LEU | C | 9 | 12.617 | 31.964 | 38.445 | 1.00 | 249.13 |
| 47 | N | ASN | C | 10 | 11.667 | 32.389 | 36.451 | 1.00 | 171.60 |
| 48 | CA | ASN | C | 10 | 11.095 | 31.064 | 36.326 | 1.00 | 171.60 |
| 49 | CB | ASN | C | 10 | 9.847 | 30.927 | 37.201 | 1.00 | 226.23 |
| 50 | CG | ASN | C | 10 | 9.428 | 29.487 | 37.375 | 1.00 | 226.23 |
| 51 | OD1 | ASN | C | 10 | 10.163 | 28.684 | 37.948 | 1.00 | 226.23 |
| 52 | ND2 | ASN | C | 10 | 8.251 | 29.146 | 36.870 | 1.00 | 226.23 |
| 53 | C | ASN | C | 10 | 10.724 | 30.744 | 34.882 | 1.00 | 171.60 |
| 54 | O | ASN | C | 10 | 9.817 | 31.353 | 34.315 | 1.00 | 171.60 |
| 55 | N | PRO | C | 11 | 11.452 | 29.806 | 34.238 | 1.00 | 202.18 |
| 56 | CD | PRO | C | 11 | 11.153 | 29.449 | 32.850 | 1.00 | 161.79 |
| 57 | CA | PRO | C | 11 | 12.551 | 28.981 | 34.761 | 1.00 | 202.18 |
| 58 | CB | PRO | C | 11 | 13.028 | 28.248 | 33.517 | 1.00 | 161.79 |
| 59 | CG | PRO | C | 11 | 11.770 | 28.086 | 32.742 | 1.00 | 161.79 |
| 60 | C | PRO | C | 11 | 13.687 | 29.788 | 35.394 | 1.00 | 202.18 |
| 61 | O | PRO | C | 11 | 13.753 | 31.010 | 35.265 | 1.00 | 202.18 |
| 62 | N | PRO | C | 12 | 14.598 | 29.101 | 36.104 | 1.00 | 182.42 |
| 63 | CD | PRO | C | 12 | 14.562 | 27.680 | 36.472 | 1.00 | 171.80 |
| 64 | CA | PRO | C | 12 | 15.721 | 29.778 | 36.762 | 1.00 | 182.42 |
| 65 | CB | PRO | C | 12 | 16.307 | 28.681 | 37.663 | 1.00 | 171.80 |
| 66 | CG | PRO | C | 12 | 15.169 | 27.708 | 37.846 | 1.00 | 171.80 |
| 67 | C | PRO | C | 12 | 16.722 | 30.257 | 35.712 | 1.00 | 182.42 |
| 68 | O | PRO | C | 12 | 17.453 | 31.230 | 35.923 | 1.00 | 182.42 |
| 69 | N | TRP | C | 13 | 16.730 | 29.550 | 34.584 | 1.00 | 151.94 |
| 70 | CA | TRP | C | 13 | 17.611 | 29.809 | 33.436 | 1.00 | 151.94 |
| 71 | CB | TRP | C | 13 | 17.185 | 28.895 | 32.289 | 1.00 | 165.82 |
| 72 | CG | TRP | C | 13 | 17.027 | 27.463 | 32.702 | 1.00 | 165.82 |
| 73 | CD2 | TRP | C | 13 | 17.776 | 26.791 | 33.712 | 1.00 | 165.82 |
| 74 | CE2 | TRP | C | 13 | 17.299 | 25.464 | 33.766 | 1.00 | 165.82 |
| 75 | CE3 | TRP | C | 13 | 18.805 | 27.183 | 34.579 | 1.00 | 165.82 |
| 76 | CD1 | TRP | C | 13 | 16.156 | 26.543 | 32.188 | 1.00 | 165.82 |
| 77 | NE1 | TRP | C | 13 | 16.314 | 25.336 | 32.821 | 1.00 | 165.82 |
| 78 | CZ2 | TRP | C | 13 | 17.815 | 24.525 | 34.659 | 1.00 | 165.82 |
| 79 | CZ3 | TRP | C | 13 | 19.320 | 26.256 | 35.464 | 1.00 | 165.82 |
| 80 | CH2 | TRP | C | 13 | 18.823 | 24.940 | 35.500 | 1.00 | 165.82 |
| 81 | C | TRP | C | 13 | 17.566 | 31.249 | 32.961 | 1.00 | 151.94 |
| 82 | O | TRP | C | 13 | 16.525 | 31.704 | 32.481 | 1.00 | 151.94 |
| 83 | N | ASN | C | 14 | 18.689 | 31.956 | 33.060 | 1.00 | 109.70 |
| 84 | CA | ASN | C | 14 | 18.712 | 33.359 | 32.634 | 1.00 | 109.70 |
| 85 | CB | ASN | C | 14 | 19.343 | 34.241 | 33.714 | 1.00 | 189.16 |
| 86 | CG | ASN | C | 14 | 20.795 | 33.911 | 33.958 | 1.00 | 189.16 |
| 87 | OD1 | ASN | C | 14 | 21.146 | 32.771 | 34.277 | 1.00 | 189.16 |
| 88 | ND2 | ASN | C | 14 | 21.656 | 34.913 | 33.812 | 1.00 | 189.16 |
| 89 | C | ASN | C | 14 | 19.434 | 33.562 | 31.296 | 1.00 | 109.70 |
| 90 | O | ASN | C | 14 | 19.917 | 34.660 | 30.972 | 1.00 | 109.70 |
| 91 | N | ARG | C | 15 | 19.490 | 32.477 | 30.524 | 1.00 | 195.68 |
| 92 | CA | ARG | C | 15 | 20.095 | 32.443 | 29.188 | 1.00 | 195.68 |
| 93 | CB | ARG | C | 15 | 21.443 | 31.715 | 29.200 | 1.00 | 140.72 |
| 94 | CG | ARG | C | 15 | 22.458 | 32.166 | 30.254 | 1.00 | 140.72 |
| 95 | CD | ARG | C | 15 | 23.806 | 31.453 | 30.030 | 1.00 | 140.72 |
| 96 | NE | ARG | C | 15 | 24.581 | 32.035 | 28.924 | 1.00 | 140.72 |
| 97 | CZ | ARG | C | 15 | 25.331 | 31.329 | 28.082 | 1.00 | 140.72 |
| 98 | NH1 | ARG | C | 15 | 25.419 | 30.009 | 28.192 | 1.00 | 140.72 |
| 99 | NH2 | ARG | C | 15 | 26.009 | 31.945 | 27.140 | 1.00 | 140.72 |
| 100 | C | ARG | C | 15 | 19.108 | 31.603 | 28.383 | 1.00 | 195.68 |
| 101 | O | ARG | C | 15 | 19.088 | 30.381 | 28.503 | 1.00 | 195.68 |
| 102 | N | ILE | C | 16 | 18.293 | 32.239 | 27.561 | 1.00 | 140.34 |
| 103 | CA | ILE | C | 16 | 17.297 | 31.485 | 26.804 | 1.00 | 140.34 |
| 104 | CB | ILE | C | 16 | 15.887 | 31.866 | 27.249 | 1.00 | 206.77 |
| 105 | CG2 | ILE | C | 16 | 15.573 | 31.233 | 28.597 | 1.00 | 206.77 |
| 106 | CG1 | ILE | C | 16 | 15.773 | 33.396 | 27.268 | 1.00 | 206.77 |
| 107 | CD1 | ILE | C | 16 | 14.370 | 33.921 | 27.429 | 1.00 | 206.77 |
| 108 | C | ILE | C | 16 | 17.327 | 31.634 | 25.280 | 1.00 | 140.34 |
| 109 | O | ILE | C | 16 | 17.796 | 32.633 | 24.729 | 1.00 | 140.34 |
| 110 | N | PHE | C | 17 | 16.789 | 30.629 | 24.604 | 1.00 | 146.56 |
| 111 | CA | PHE | C | 17 | 16.713 | 30.628 | 23.155 | 1.00 | 146.56 |
| 112 | CB | PHE | C | 17 | 16.294 | 29.246 | 22.661 | 1.00 | 145.27 |
| 113 | CG | PHE | C | 17 | 17.440 | 28.331 | 22.377 | 1.00 | 145.27 |
| 114 | CD1 | PHE | C | 17 | 17.332 | 26.958 | 22.623 | 1.00 | 145.27 |
| 115 | CD2 | PHE | C | 17 | 18.618 | 28.832 | 21.834 | 1.00 | 145.27 |
| 116 | CE1 | PHE | C | 17 | 18.377 | 26.099 | 22.333 | 1.00 | 145.27 |
| 117 | CE2 | PHE | C | 17 | 19.673 | 27.979 | 21.537 | 1.00 | 145.27 |
| 118 | CZ | PHE | C | 17 | 19.554 | 26.604 | 21.788 | 1.00 | 145.27 |
| 119 | C | PHE | C | 17 | 15.690 | 31.647 | 22.693 | 1.00 | 146.56 |
| 120 | O | PHE | C | 17 | 15.030 | 32.293 | 23.492 | 1.00 | 146.56 |
| 121 | N | LYS | C | 18 | 15.555 | 31.769 | 21.382 | 1.00 | 131.41 |
| 122 | CA | LYS | C | 18 | 14.614 | 32.698 | 20.755 | 1.00 | 131.41 |
| 123 | CB | LYS | C | 18 | 15.113 | 33.048 | 19.348 | 1.00 | 248.17 |
| 124 | CG | LYS | C | 18 | 14.275 | 34.053 | 18.584 | 1.00 | 248.17 |
| 125 | CD | LYS | C | 18 | 14.973 | 34.434 | 17.285 | 1.00 | 248.17 |
| 126 | CE | LYS | C | 18 | 14.134 | 35.379 | 16.440 | 1.00 | 248.17 |
| 127 | NZ | LYS | C | 18 | 12.913 | 34.721 | 15.900 | 1.00 | 248.17 |
| 128 | C | LYS | C | 18 | 13.203 | 32.089 | 20.684 | 1.00 | 131.41 |
| 129 | O | LYS | C | 18 | 13.013 | 30.957 | 20.227 | 1.00 | 131.41 |
| 130 | N | GLY | C | 19 | 12.218 | 32.849 | 21.159 | 1.00 | 243.18 |
| 131 | CA | GLY | C | 19 | 10.842 | 32.386 | 21.134 | 1.00 | 243.18 |
| 132 | C | GLY | C | 19 | 10.346 | 31.737 | 22.415 | 1.00 | 243.18 |
| 133 | O | GLY | C | 19 | 9.146 | 31.500 | 22.566 | 1.00 | 243.18 |
| 134 | N | GLU | C | 20 | 11.256 | 31.447 | 23.341 | 1.00 | 154.05 |
| 135 | CA | GLU | C | 20 | 10.892 | 30.810 | 24.615 | 1.00 | 154.05 |
| 136 | CB | GLU | C | 20 | 12.136 | 30.161 | 25.259 | 1.00 | 176.57 |
| 137 | CG | GLU | C | 20 | 12.994 | 29.290 | 24.335 | 1.00 | 176.57 |
| 138 | CD | GLU | C | 20 | 14.115 | 28.594 | 25.077 | 1.00 | 176.57 |
| 139 | OE1 | GLU | C | 20 | 14.898 | 29.282 | 25.777 | 1.00 | 176.57 |
| 140 | OE2 | GLU | C | 20 | 14.217 | 27.356 | 24.959 | 1.00 | 176.57 |
| 141 | C | GLU | C | 20 | 10.297 | 31.833 | 25.582 | 1.00 | 154.05 |
| 142 | O | GLU | C | 20 | 10.532 | 33.032 | 25.419 | 1.00 | 154.05 |
| 143 | N | ASN | C | 21 | 9.550 | 31.365 | 26.587 | 1.00 | 173.20 |
| 144 | CA | ASN | C | 21 | 8.957 | 32.290 | 27.559 | 1.00 | 173.20 |
| 145 | CB | ASN | C | 21 | 7.446 | 32.074 | 27.682 | 1.00 | 249.69 |
| 146 | CG | ASN | C | 21 | 6.794 | 31.675 | 26.378 | 1.00 | 249.69 |
| 147 | OD1 | ASN | C | 21 | 7.014 | 32.277 | 25.326 | 1.00 | 249.69 |
| 148 | ND2 | ASN | C | 21 | 5.961 | 30.647 | 26.472 | 1.00 | 249.69 |
| 149 | C | ASN | C | 21 | 9.559 | 32.227 | 28.975 | 1.00 | 173.20 |
| 150 | O | ASN | C | 21 | 9.892 | 31.148 | 29.474 | 1.00 | 173.20 |
| 151 | N | VAL | C | 22 | 9.661 | 33.393 | 29.617 | 1.00 | 186.44 |
| 152 | CA | VAL | C | 22 | 10.209 | 33.508 | 30.964 | 1.00 | 186.44 |
| 153 | CB | VAL | C | 22 | 11.664 | 34.016 | 30.926 | 1.00 | 163.28 |
| 154 | CG1 | VAL | C | 22 | 11.701 | 35.486 | 30.538 | 1.00 | 163.28 |
| 155 | CG2 | VAL | C | 22 | 12.315 | 33.802 | 32.273 | 1.00 | 163.28 |
| 156 | C | VAL | C | 22 | 9.379 | 34.489 | 31.797 | 1.00 | 186.44 |
| 157 | O | VAL | C | 22 | 8.852 | 35.463 | 31.271 | 1.00 | 186.44 |
| 158 | N | THR | C | 23 | 9.289 | 34.241 | 33.102 | 1.00 | 165.76 |
| 159 | CA | THR | C | 23 | 8.512 | 35.092 | 34.014 | 1.00 | 165.76 |
| 160 | CB | THR | C | 23 | 7.425 | 34.263 | 34.728 | 1.00 | 249.09 |
| 161 | OG1 | THR | C | 23 | 6.671 | 33.521 | 33.760 | 1.00 | 249.09 |
| 162 | CG2 | THR | C | 23 | 6.492 | 35.177 | 35.511 | 1.00 | 249.09 |
| 163 | C | THR | C | 23 | 9.348 | 35.780 | 35.098 | 1.00 | 165.76 |
| 164 | O | THR | C | 23 | 10.061 | 35.119 | 35.850 | 1.00 | 165.76 |
| 165 | N | LEU | C | 24 | 9.239 | 37.099 | 35.195 | 1.00 | 173.95 |
| 166 | CA | LEU | C | 24 | 9.990 | 37.842 | 36.206 | 1.00 | 173.95 |
| 167 | CB | LEU | C | 24 | 10.661 | 39.079 | 35.589 | 1.00 | 128.36 |
| 168 | CG | LEU | C | 24 | 11.163 | 39.097 | 34.140 | 1.00 | 128.36 |
| 169 | CD1 | LEU | C | 24 | 12.080 | 40.307 | 33.939 | 1.00 | 128.36 |
| 170 | CD2 | LEU | C | 24 | 11.903 | 37.824 | 33.821 | 1.00 | 128.36 |
| 171 | C | LEU | C | 24 | 9.089 | 38.297 | 37.365 | 1.00 | 173.95 |
| 172 | O | LEU | C | 24 | 8.276 | 39.207 | 37.208 | 1.00 | 173.95 |
| 173 | N | THR | C | 25 | 9.249 | 37.669 | 38.526 | 1.00 | 172.54 |
| 174 | CA | THR | C | 25 | 8.463 | 37.995 | 39.717 | 1.00 | 172.54 |
| 175 | CB | THR | C | 25 | 8.096 | 36.712 | 40.504 | 1.00 | 195.25 |
| 176 | OG1 | THR | C | 25 | 7.369 | 35.824 | 39.645 | 1.00 | 195.25 |
| 177 | CG2 | THR | C | 25 | 7.244 | 37.045 | 41.724 | 1.00 | 195.25 |
| 178 | C | THR | C | 25 | 9.253 | 38.923 | 40.636 | 1.00 | 172.54 |
| 179 | O | THR | C | 25 | 10.427 | 38.681 | 40.895 | 1.00 | 172.54 |
| 180 | N | CYS | C | 26 | 8.610 | 39.978 | 41.130 | 1.00 | 199.84 |
| 181 | CA | CYS | C | 26 | 9.269 | 40.937 | 42.025 | 1.00 | 199.84 |
| 182 | C | CYS | C | 26 | 9.272 | 40.407 | 43.458 | 1.00 | 199.84 |
| 183 | O | CYS | C | 26 | 8.303 | 39.775 | 43.889 | 1.00 | 199.84 |
| 184 | CB | CYS | C | 26 | 8.556 | 42.292 | 41.955 | 1.00 | 211.93 |
| 185 | SG | CYS | C | 26 | 9.426 | 43.668 | 42.769 | 1.00 | 211.93 |
| 186 | N | ASN | C | 27 | 10.358 | 40.673 | 44.186 | 1.00 | 249.36 |
| 187 | CA | ASN | C | 27 | 10.531 | 40.203 | 45.564 | 1.00 | 249.36 |
| 188 | CB | ASN | C | 27 | 11.176 | 41.291 | 46.437 | 1.00 | 249.69 |
| 189 | CG | ASN | C | 27 | 11.614 | 40.764 | 47.804 | 1.00 | 249.69 |
| 190 | OD1 | ASN | C | 27 | 12.279 | 39.728 | 47.907 | 1.00 | 249.69 |
| 191 | ND2 | ASN | C | 27 | 11.246 | 41.481 | 48.858 | 1.00 | 249.69 |
| 192 | C | ASN | C | 27 | 9.245 | 39.705 | 46.225 | 1.00 | 249.36 |
| 193 | O | ASN | C | 27 | 8.484 | 40.481 | 46.815 | 1.00 | 249.36 |
| 194 | N | GLY | C | 28 | 9.029 | 38.395 | 46.116 | 1.00 | 249.69 |
| 195 | CA | GLY | C | 28 | 7.858 | 37.746 | 46.685 | 1.00 | 249.69 |
| 196 | C | GLY | C | 28 | 7.872 | 36.313 | 46.199 | 1.00 | 249.69 |
| 197 | O | GLY | C | 28 | 7.839 | 36.074 | 44.991 | 1.00 | 249.69 |
| 198 | N | ASN | C | 29 | 7.927 | 35.361 | 47.129 | 1.00 | 249.69 |
| 199 | CA | ASN | C | 29 | 7.980 | 33.942 | 46.771 | 1.00 | 249.69 |
| 200 | CB | ASN | C | 29 | 8.454 | 33.111 | 47.988 | 1.00 | 249.69 |
| 201 | CG | ASN | C | 29 | 8.804 | 31.655 | 47.627 | 1.00 | 249.69 |
| 202 | OD1 | ASN | C | 29 | 8.854 | 31.278 | 46.450 | 1.00 | 249.69 |
| 203 | ND2 | ASN | C | 29 | 9.055 | 30.840 | 48.650 | 1.00 | 249.69 |
| 204 | C | ASN | C | 29 | 6.655 | 33.386 | 46.224 | 1.00 | 249.69 |
| 205 | O | ASN | C | 29 | 6.633 | 32.784 | 45.140 | 1.00 | 249.69 |
| 206 | N | ASN | C | 30 | 5.554 | 33.594 | 46.946 | 1.00 | 249.69 |
| 207 | CA | ASN | C | 30 | 4.270 | 33.055 | 46.497 | 1.00 | 249.69 |
| 208 | CB | ASN | C | 30 | 3.852 | 31.902 | 47.424 | 1.00 | 249.69 |
| 209 | CG | ASN | C | 30 | 4.822 | 30.717 | 47.372 | 1.00 | 249.69 |
| 210 | OD1 | ASN | C | 30 | 5.230 | 30.182 | 48.410 | 1.00 | 249.69 |
| 211 | ND2 | ASN | C | 30 | 5.186 | 30.299 | 46.163 | 1.00 | 249.69 |
| 212 | C | ASN | C | 30 | 3.119 | 34.055 | 46.361 | 1.00 | 249.69 |
| 213 | O | ASN | C | 30 | 2.662 | 34.325 | 45.248 | 1.00 | 249.69 |
| 214 | N | PHE | C | 31 | 2.650 | 34.602 | 47.482 | 1.00 | 249.69 |
| 215 | CA | PHE | C | 31 | 1.531 | 35.546 | 47.446 | 1.00 | 249.69 |
| 216 | CB | PHE | C | 31 | 0.361 | 35.003 | 48.290 | 1.00 | 249.52 |
| 217 | CG | PHE | C | 31 | −0.075 | 33.609 | 47.903 | 1.00 | 249.52 |
| 218 | CD1 | PHE | C | 31 | 0.636 | 32.498 | 48.348 | 1.00 | 249.52 |
| 219 | CD2 | PHE | C | 31 | −1.176 | 33.411 | 47.071 | 1.00 | 249.52 |
| 220 | CE1 | PHE | C | 31 | 0.261 | 31.211 | 47.966 | 1.00 | 249.52 |
| 221 | CE2 | PHE | C | 31 | −1.557 | 32.128 | 46.684 | 1.00 | 249.52 |
| 222 | CZ | PHE | C | 31 | −0.838 | 31.026 | 47.132 | 1.00 | 249.52 |
| 223 | C | PHE | C | 31 | 1.872 | 36.984 | 47.884 | 1.00 | 249.69 |
| 224 | O | PHE | C | 31 | 2.350 | 37.221 | 49.003 | 1.00 | 249.69 |
| 225 | N | PHE | C | 32 | 1.605 | 37.936 | 46.986 | 1.00 | 249.62 |
| 226 | CA | PHE | C | 32 | 1.872 | 39.354 | 47.227 | 1.00 | 249.62 |
| 227 | CB | PHE | C | 32 | 2.862 | 39.873 | 46.176 | 1.00 | 249.69 |
| 228 | CG | PHE | C | 32 | 3.487 | 41.203 | 46.520 | 1.00 | 249.69 |
| 229 | CD1 | PHE | C | 32 | 4.351 | 41.325 | 47.611 | 1.00 | 249.69 |
| 230 | CD2 | PHE | C | 32 | 3.224 | 42.334 | 45.741 | 1.00 | 249.69 |
| 231 | CE1 | PHE | C | 32 | 4.948 | 42.554 | 47.918 | 1.00 | 249.69 |
| 232 | CE2 | PHE | C | 32 | 3.814 | 43.566 | 46.039 | 1.00 | 249.69 |
| 233 | CZ | PHE | C | 32 | 4.678 | 43.673 | 47.130 | 1.00 | 249.69 |
| 234 | C | PHE | C | 32 | 0.569 | 40.161 | 47.176 | 1.00 | 249.62 |
| 235 | O | PHE | C | 32 | −0.470 | 39.650 | 46.738 | 1.00 | 249.62 |
| 236 | N | GLU | C | 33 | 0.636 | 41.424 | 47.595 | 1.00 | 238.93 |
| 237 | CA | GLU | C | 33 | −0.554 | 42.273 | 47.631 | 1.00 | 238.93 |
| 238 | CB | GLU | C | 33 | −0.811 | 42.705 | 49.079 | 1.00 | 249.69 |
| 239 | CG | GLU | C | 33 | −2.234 | 43.193 | 49.339 | 1.00 | 249.69 |
| 240 | CD | GLU | C | 33 | −3.285 | 42.246 | 48.762 | 1.00 | 249.69 |
| 241 | OE1 | GLU | C | 33 | −3.144 | 41.010 | 48.947 | 1.00 | 249.69 |
| 242 | OE2 | GLU | C | 33 | −4.250 | 42.733 | 48.124 | 1.00 | 249.69 |
| 243 | C | GLU | C | 33 | −0.613 | 43.512 | 46.721 | 1.00 | 238.93 |
| 244 | O | GLU | C | 33 | −1.589 | 43.716 | 45.998 | 1.00 | 238.93 |
| 245 | N | VAL | C | 34 | 0.420 | 44.344 | 46.762 | 1.00 | 237.42 |
| 246 | CA | VAL | C | 34 | 0.452 | 45.563 | 45.959 | 1.00 | 237.42 |
| 247 | CB | VAL | C | 34 | 1.760 | 46.350 | 46.235 | 1.00 | 249.69 |
| 248 | CG1 | VAL | C | 34 | 1.775 | 47.644 | 45.447 | 1.00 | 249.69 |
| 249 | CG2 | VAL | C | 34 | 1.875 | 46.644 | 47.726 | 1.00 | 249.69 |
| 250 | C | VAL | C | 34 | 0.284 | 45.376 | 44.447 | 1.00 | 237.42 |
| 251 | O | VAL | C | 34 | 0.665 | 44.351 | 43.880 | 1.00 | 237.42 |
| 252 | N | SER | C | 35 | −0.305 | 46.386 | 43.812 | 1.00 | 249.64 |
| 253 | CA | SER | C | 35 | −0.535 | 46.390 | 42.370 | 1.00 | 249.64 |
| 254 | CB | SER | C | 35 | −1.976 | 46.787 | 42.058 | 1.00 | 249.69 |
| 255 | OG | SER | C | 35 | −2.186 | 48.165 | 42.327 | 1.00 | 249.69 |
| 256 | C | SER | C | 35 | 0.403 | 47.409 | 41.729 | 1.00 | 249.64 |
| 257 | O | SER | C | 35 | 0.418 | 47.573 | 40.504 | 1.00 | 249.64 |
| 258 | N | SER | C | 36 | 1.171 | 48.101 | 42.573 | 1.00 | 249.69 |
| 259 | CA | SER | C | 36 | 2.129 | 49.109 | 42.112 | 1.00 | 249.69 |
| 260 | CB | SER | C | 36 | 2.054 | 50.374 | 42.987 | 1.00 | 249.69 |
| 261 | OG | SER | C | 36 | 2.599 | 50.160 | 44.280 | 1.00 | 249.69 |
| 262 | C | SER | C | 36 | 3.555 | 48.551 | 42.130 | 1.00 | 249.69 |
| 263 | O | SER | C | 36 | 4.261 | 48.626 | 43.142 | 1.00 | 249.69 |
| 264 | N | THR | C | 37 | 3.961 | 47.977 | 40.999 | 1.00 | 198.99 |
| 265 | CA | THR | C | 37 | 5.286 | 47.408 | 40.863 | 1.00 | 198.99 |
| 266 | CB | THR | C | 37 | 5.205 | 45.867 | 40.697 | 1.00 | 176.65 |
| 267 | OG1 | THR | C | 37 | 4.557 | 45.280 | 41.840 | 1.00 | 176.65 |
| 268 | CG2 | THR | C | 37 | 6.597 | 45.275 | 40.573 | 1.00 | 176.65 |
| 269 | C | THR | C | 37 | 5.905 | 48.053 | 39.632 | 1.00 | 198.99 |
| 270 | O | THR | C | 37 | 5.232 | 48.246 | 38.619 | 1.00 | 198.99 |
| 271 | N | LYS | C | 38 | 7.182 | 48.400 | 39.723 | 1.00 | 249.69 |
| 272 | CA | LYS | C | 38 | 7.865 | 49.041 | 38.606 | 1.00 | 249.69 |
| 273 | CB | LYS | C | 38 | 8.609 | 50.287 | 39.109 | 1.00 | 249.38 |
| 274 | CG | LYS | C | 38 | 7.697 | 51.314 | 39.792 | 1.00 | 249.38 |
| 275 | CD | LYS | C | 38 | 8.467 | 52.537 | 40.303 | 1.00 | 249.38 |
| 276 | CE | LYS | C | 38 | 7.527 | 53.572 | 40.930 | 1.00 | 249.38 |
| 277 | NZ | LYS | C | 38 | 8.240 | 54.792 | 41.414 | 1.00 | 249.38 |
| 278 | C | LYS | C | 38 | 8.837 | 48.092 | 37.894 | 1.00 | 249.69 |
| 279 | O | LYS | C | 38 | 9.473 | 47.247 | 38.519 | 1.00 | 249.69 |
| 280 | N | TRP | C | 39 | 8.933 | 48.221 | 36.576 | 1.00 | 205.23 |
| 281 | CA | TRP | C | 39 | 9.837 | 47.391 | 35.790 | 1.00 | 205.23 |
| 282 | CB | TRP | C | 39 | 9.052 | 46.417 | 34.916 | 1.00 | 163.48 |
| 283 | CG | TRP | C | 39 | 8.273 | 45.376 | 35.653 | 1.00 | 163.48 |
| 284 | CD2 | TRP | C | 39 | 8.795 | 44.365 | 36.525 | 1.00 | 163.48 |
| 285 | CE2 | TRP | C | 39 | 7.715 | 43.525 | 36.893 | 1.00 | 163.48 |
| 286 | CE3 | TRP | C | 39 | 10.069 | 44.083 | 37.032 | 1.00 | 163.48 |
| 287 | CD1 | TRP | C | 39 | 6.939 | 45.125 | 35.542 | 1.00 | 163.48 |
| 288 | NE1 | TRP | C | 39 | 6.591 | 44.013 | 36.278 | 1.00 | 163.48 |
| 289 | CZ2 | TRP | C | 39 | 7.866 | 42.419 | 37.737 | 1.00 | 163.48 |
| 290 | CZ3 | TRP | C | 39 | 10.225 | 42.976 | 37.881 | 1.00 | 163.48 |
| 291 | CH2 | TRP | C | 39 | 9.125 | 42.162 | 38.220 | 1.00 | 163.48 |
| 292 | C | TRP | C | 39 | 10.637 | 48.332 | 34.908 | 1.00 | 205.23 |
| 293 | O | TRP | C | 39 | 10.076 | 49.233 | 34.280 | 1.00 | 205.23 |
| 294 | N | PHE | C | 40 | 11.947 | 48.138 | 34.857 | 1.00 | 127.08 |
| 295 | CA | PHE | C | 40 | 12.800 | 49.016 | 34.034 | 1.00 | 127.08 |
| 296 | CB | PHE | C | 40 | 13.686 | 49.895 | 34.930 | 1.00 | 249.69 |
| 297 | CG | PHE | C | 40 | 12.922 | 50.766 | 35.900 | 1.00 | 249.69 |
| 298 | CD1 | PHE | C | 40 | 12.431 | 50.242 | 37.097 | 1.00 | 249.69 |
| 299 | CD2 | PHE | C | 40 | 12.724 | 52.121 | 35.630 | 1.00 | 249.69 |
| 300 | CE1 | PHE | C | 40 | 11.762 | 51.055 | 38.010 | 1.00 | 249.69 |
| 301 | CE2 | PHE | C | 40 | 12.054 | 52.941 | 36.539 | 1.00 | 249.69 |
| 302 | CZ | PHE | C | 40 | 11.574 | 52.408 | 37.731 | 1.00 | 249.69 |
| 303 | C | PHE | C | 40 | 13.714 | 48.294 | 33.012 | 1.00 | 127.08 |
| 304 | O | PHE | C | 40 | 14.938 | 48.204 | 33.191 | 1.00 | 127.08 |
| 305 | N | HIS | C | 41 | 13.118 | 47.801 | 31.936 | 1.00 | 117.94 |
| 306 | CA | HIS | C | 41 | 13.846 | 47.101 | 30.884 | 1.00 | 117.94 |
| 307 | CB | HIS | C | 41 | 12.846 | 46.566 | 29.848 | 1.00 | 198.34 |
| 308 | CG | HIS | C | 41 | 13.482 | 45.817 | 28.723 | 1.00 | 198.34 |
| 309 | CD2 | HIS | C | 41 | 13.214 | 45.791 | 27.395 | 1.00 | 198.34 |
| 310 | ND1 | HIS | C | 41 | 14.515 | 44.930 | 28.924 | 1.00 | 198.34 |
| 311 | CE1 | HIS | C | 41 | 14.856 | 44.390 | 27.769 | 1.00 | 198.34 |
| 312 | NE2 | HIS | C | 41 | 14.082 | 44.895 | 26.826 | 1.00 | 198.34 |
| 313 | C | HIS | C | 41 | 14.863 | 48.015 | 30.192 | 1.00 | 117.94 |
| 314 | O | HIS | C | 41 | 14.509 | 48.859 | 29.389 | 1.00 | 117.94 |
| 315 | N | ASN | C | 42 | 16.135 | 47.813 | 30.481 | 1.00 | 147.15 |
| 316 | CA | ASN | C | 42 | 17.216 | 48.618 | 29.912 | 1.00 | 147.15 |
| 317 | CB | ASN | C | 42 | 17.135 | 48.679 | 28.370 | 1.00 | 208.25 |
| 318 | CG | ASN | C | 42 | 17.652 | 47.411 | 27.699 | 1.00 | 208.25 |
| 319 | OD1 | ASN | C | 42 | 17.253 | 46.309 | 28.074 | 1.00 | 208.25 |
| 320 | ND2 | ASN | C | 42 | 18.527 | 47.562 | 26.702 | 1.00 | 208.25 |
| 321 | C | ASN | C | 42 | 17.140 | 50.019 | 30.506 | 1.00 | 147.15 |
| 322 | O | ASN | C | 42 | 17.627 | 50.986 | 29.917 | 1.00 | 147.15 |
| 323 | N | GLY | C | 43 | 16.527 | 50.115 | 31.683 | 1.00 | 230.72 |
| 324 | CA | GLY | C | 43 | 16.372 | 51.400 | 32.344 | 1.00 | 230.72 |
| 325 | C | GLY | C | 43 | 15.019 | 52.031 | 32.048 | 1.00 | 230.72 |
| 326 | O | GLY | C | 43 | 14.369 | 52.590 | 32.933 | 1.00 | 230.72 |
| 327 | N | SER | C | 44 | 14.596 | 51.937 | 30.790 | 1.00 | 208.53 |
| 328 | CA | SER | C | 44 | 13.320 | 52.490 | 30.334 | 1.00 | 208.53 |
| 329 | CB | SER | C | 44 | 13.133 | 52.231 | 28.833 | 1.00 | 178.10 |
| 330 | OG | SER | C | 44 | 14.168 | 52.830 | 28.070 | 1.00 | 178.10 |
| 331 | C | SER | C | 44 | 12.146 | 51.881 | 31.079 | 1.00 | 208.53 |
| 332 | O | SER | C | 44 | 11.961 | 50.670 | 31.066 | 1.00 | 208.53 |
| 333 | N | LEU | C | 45 | 11.338 | 52.719 | 31.713 | 1.00 | 211.15 |
| 334 | CA | LEU | C | 45 | 10.186 | 52.214 | 32.442 | 1.00 | 211.15 |
| 335 | CB | LEU | C | 45 | 9.346 | 53.372 | 32.985 | 1.00 | 239.89 |
| 336 | CG | LEU | C | 45 | 8.132 | 52.948 | 33.821 | 1.00 | 239.89 |
| 337 | CD1 | LEU | C | 45 | 8.571 | 52.034 | 34.952 | 1.00 | 239.89 |
| 338 | CD2 | LEU | C | 45 | 7.433 | 54.178 | 34.368 | 1.00 | 239.89 |
| 339 | C | LEU | C | 45 | 9.330 | 51.325 | 31.540 | 1.00 | 211.15 |
| 340 | O | LEU | C | 45 | 9.278 | 51.528 | 30.323 | 1.00 | 211.15 |
| 341 | N | SER | C | 46 | 8.669 | 50.339 | 32.143 | 1.00 | 166.46 |
| 342 | CA | SER | C | 46 | 7.826 | 49.404 | 31.400 | 1.00 | 166.46 |
| 343 | CB | SER | C | 46 | 8.138 | 47.964 | 31.815 | 1.00 | 249.69 |
| 344 | OG | SER | C | 46 | 7.394 | 47.032 | 31.043 | 1.00 | 249.69 |
| 345 | C | SER | C | 46 | 6.345 | 49.671 | 31.608 | 1.00 | 166.46 |
| 346 | O | SER | C | 46 | 5.973 | 50.451 | 32.488 | 1.00 | 166.46 |
| 347 | N | GLU | C | 47 | 5.512 | 48.996 | 30.813 | 1.00 | 202.45 |
| 348 | CA | GLU | C | 47 | 4.064 | 49.168 | 30.864 | 1.00 | 202.45 |
| 349 | CB | GLU | C | 47 | 3.485 | 49.010 | 29.458 | 1.00 | 249.69 |
| 350 | CG | GLU | C | 47 | 4.000 | 50.047 | 28.469 | 1.00 | 249.69 |
| 351 | CD | GLU | C | 47 | 3.429 | 49.860 | 27.078 | 1.00 | 249.69 |
| 352 | OE1 | GLU | C | 47 | 3.693 | 48.805 | 26.462 | 1.00 | 249.69 |
| 353 | OE2 | GLU | C | 47 | 2.715 | 50.769 | 26.600 | 1.00 | 249.69 |
| 354 | C | GLU | C | 47 | 3.296 | 48.271 | 31.832 | 1.00 | 202.45 |
| 355 | O | GLU | C | 47 | 2.108 | 48.506 | 32.090 | 1.00 | 202.45 |
| 356 | N | GLU | C | 48 | 3.948 | 47.243 | 32.361 | 1.00 | 214.28 |
| 357 | CA | GLU | C | 48 | 3.264 | 46.372 | 33.301 | 1.00 | 214.28 |
| 358 | CB | GLU | C | 48 | 3.882 | 44.973 | 33.294 | 1.00 | 197.36 |
| 359 | CG | GLU | C | 48 | 3.286 | 44.027 | 34.340 | 1.00 | 197.36 |
| 360 | CD | GLU | C | 48 | 1.825 | 43.715 | 34.097 | 1.00 | 197.36 |
| 361 | OE1 | GLU | C | 48 | 1.535 | 42.984 | 33.130 | 1.00 | 197.36 |
| 362 | OE2 | GLU | C | 48 | 0.964 | 44.203 | 34.866 | 1.00 | 197.36 |
| 363 | C | GLU | C | 48 | 3.343 | 46.977 | 34.702 | 1.00 | 214.28 |
| 364 | O | GLU | C | 48 | 4.236 | 47.788 | 34.995 | 1.00 | 214.28 |
| 365 | N | THR | C | 49 | 2.398 | 46.584 | 35.557 | 1.00 | 211.95 |
| 366 | CA | THR | C | 49 | 2.335 | 47.069 | 36.932 | 1.00 | 211.95 |
| 367 | CB | THR | C | 49 | 1.126 | 48.003 | 37.123 | 1.00 | 249.69 |
| 368 | OG1 | THR | C | 49 | −0.069 | 47.327 | 36.706 | 1.00 | 249.69 |
| 369 | CG2 | THR | C | 49 | 1.305 | 49.278 | 36.301 | 1.00 | 249.69 |
| 370 | C | THR | C | 49 | 2.220 | 45.895 | 37.901 | 1.00 | 211.95 |
| 371 | O | THR | C | 49 | 2.631 | 45.988 | 39.055 | 1.00 | 211.95 |
| 372 | N | ASN | C | 50 | 1.650 | 44.797 | 37.421 | 1.00 | 207.90 |
| 373 | CA | ASN | C | 50 | 1.502 | 43.601 | 38.234 | 1.00 | 207.90 |
| 374 | CB | ASN | C | 50 | 0.856 | 42.486 | 37.403 | 1.00 | 210.82 |
| 375 | CG | ASN | C | 50 | 0.443 | 41.295 | 38.245 | 1.00 | 210.82 |
| 376 | OD1 | ASN | C | 50 | 0.925 | 41.126 | 39.365 | 1.00 | 210.82 |
| 377 | ND2 | ASN | C | 50 | −0.437 | 40.456 | 37.705 | 1.00 | 210.82 |
| 378 | C | ASN | C | 50 | 2.914 | 43.187 | 38.670 | 1.00 | 207.90 |
| 379 | O | ASN | C | 50 | 3.888 | 43.479 | 37.978 | 1.00 | 207.90 |
| 380 | N | SER | C | 51 | 3.036 | 42.509 | 39.808 | 1.00 | 249.50 |
| 381 | CA | SER | C | 51 | 4.352 | 42.086 | 40.286 | 1.00 | 249.50 |
| 382 | CB | SER | C | 51 | 4.260 | 41.569 | 41.728 | 1.00 | 249.69 |
| 383 | OG | SER | C | 51 | 3.632 | 40.295 | 41.780 | 1.00 | 249.69 |
| 384 | C | SER | C | 51 | 4.994 | 41.012 | 39.395 | 1.00 | 249.50 |
| 385 | O | SER | C | 51 | 6.196 | 40.775 | 39.483 | 1.00 | 249.50 |
| 386 | N | SER | C | 52 | 4.195 | 40.367 | 38.544 | 1.00 | 228.11 |
| 387 | CA | SER | C | 52 | 4.705 | 39.328 | 37.645 | 1.00 | 228.11 |
| 388 | CB | SER | C | 52 | 3.867 | 38.049 | 37.741 | 1.00 | 168.18 |
| 389 | OG | SER | C | 52 | 3.908 | 37.491 | 39.042 | 1.00 | 168.18 |
| 390 | C | SER | C | 52 | 4.726 | 39.783 | 36.194 | 1.00 | 228.11 |
| 391 | O | SER | C | 52 | 3.692 | 39.843 | 35.528 | 1.00 | 228.11 |
| 392 | N | LEU | C | 53 | 5.919 | 40.096 | 35.708 | 1.00 | 153.71 |
| 393 | CA | LEU | C | 53 | 6.111 | 40.542 | 34.332 | 1.00 | 153.71 |
| 394 | CB | LEU | C | 53 | 7.219 | 41.594 | 34.278 | 1.00 | 123.91 |
| 395 | CG | LEU | C | 53 | 7.891 | 41.882 | 32.939 | 1.00 | 123.91 |
| 396 | CD1 | LEU | C | 53 | 6.841 | 42.018 | 31.823 | 1.00 | 123.91 |
| 397 | CD2 | LEU | C | 53 | 8.744 | 43.157 | 33.084 | 1.00 | 123.91 |
| 398 | C | LEU | C | 53 | 6.476 | 39.373 | 33.439 | 1.00 | 153.71 |
| 399 | O | LEU | C | 53 | 7.604 | 38.887 | 33.461 | 1.00 | 153.71 |
| 400 | N | ASN | C | 54 | 5.514 | 38.918 | 32.655 | 1.00 | 221.05 |
| 401 | CA | ASN | C | 54 | 5.772 | 37.804 | 31.773 | 1.00 | 221.05 |
| 402 | CB | ASN | C | 54 | 4.474 | 37.081 | 31.431 | 1.00 | 192.59 |
| 403 | CG | ASN | C | 54 | 3.924 | 36.312 | 32.601 | 1.00 | 192.59 |
| 404 | OD1 | ASN | C | 54 | 4.626 | 35.511 | 33.211 | 1.00 | 192.59 |
| 405 | ND2 | ASN | C | 54 | 2.661 | 36.548 | 32.922 | 1.00 | 192.59 |
| 406 | C | ASN | C | 54 | 6.477 | 38.221 | 30.497 | 1.00 | 221.05 |
| 407 | O | ASN | C | 54 | 6.451 | 39.391 | 30.098 | 1.00 | 221.05 |
| 408 | N | ILE | C | 55 | 7.116 | 37.234 | 29.873 | 1.00 | 249.69 |
| 409 | CA | ILE | C | 55 | 7.850 | 37.402 | 28.624 | 1.00 | 249.69 |
| 410 | CB | ILE | C | 55 | 9.374 | 37.380 | 28.869 | 1.00 | 131.97 |
| 411 | CG2 | ILE | C | 55 | 10.103 | 36.988 | 27.599 | 1.00 | 131.97 |
| 412 | CG1 | ILE | C | 55 | 9.822 | 38.756 | 29.380 | 1.00 | 131.97 |
| 413 | CD1 | ILE | C | 55 | 11.301 | 38.863 | 29.665 | 1.00 | 131.97 |
| 414 | C | ILE | C | 55 | 7.468 | 36.235 | 27.720 | 1.00 | 249.69 |
| 415 | O | ILE | C | 55 | 7.742 | 35.080 | 28.048 | 1.00 | 249.69 |
| 416 | N | VAL | C | 56 | 6.829 | 36.531 | 26.595 | 1.00 | 201.86 |
| 417 | CA | VAL | C | 56 | 6.422 | 35.474 | 25.687 | 1.00 | 201.86 |
| 418 | CB | VAL | C | 56 | 5.043 | 35.759 | 25.089 | 1.00 | 231.54 |
| 419 | CG1 | VAL | C | 56 | 4.431 | 34.468 | 24.565 | 1.00 | 231.54 |
| 420 | CG2 | VAL | C | 56 | 4.144 | 36.385 | 26.138 | 1.00 | 231.54 |
| 421 | C | VAL | C | 56 | 7.454 | 35.345 | 24.578 | 1.00 | 201.86 |
| 422 | O | VAL | C | 56 | 8.595 | 35.775 | 24.747 | 1.00 | 201.86 |
| 423 | N | ASN | C | 57 | 7.056 | 34.758 | 23.451 | 1.00 | 157.94 |
| 424 | CA | ASN | C | 57 | 7.953 | 34.542 | 22.310 | 1.00 | 157.94 |
| 425 | CB | ASN | C | 57 | 7.179 | 34.657 | 20.994 | 1.00 | 249.57 |
| 426 | CG | ASN | C | 57 | 6.212 | 33.499 | 20.793 | 1.00 | 249.57 |
| 427 | OD1 | ASN | C | 57 | 6.593 | 32.333 | 20.911 | 1.00 | 249.57 |
| 428 | ND2 | ASN | C | 57 | 4.958 | 33.812 | 20.488 | 1.00 | 249.57 |
| 429 | C | ASN | C | 57 | 9.147 | 35.472 | 22.324 | 1.00 | 157.94 |
| 430 | O | ASN | C | 57 | 9.103 | 36.592 | 21.825 | 1.00 | 157.94 |
| 431 | N | ALA | C | 58 | 10.213 | 34.960 | 22.924 | 1.00 | 146.95 |
| 432 | CA | ALA | C | 58 | 11.477 | 35.658 | 23.112 | 1.00 | 146.95 |
| 433 | CB | ALA | C | 58 | 12.467 | 34.717 | 23.796 | 1.00 | 132.39 |
| 434 | C | ALA | C | 58 | 12.122 | 36.270 | 21.878 | 1.00 | 146.95 |
| 435 | O | ALA | C | 58 | 12.657 | 35.566 | 21.014 | 1.00 | 146.95 |
| 436 | N | LYS | C | 59 | 12.087 | 37.596 | 21.816 | 1.00 | 135.91 |
| 437 | CA | LYS | C | 59 | 12.680 | 38.350 | 20.710 | 1.00 | 135.91 |
| 438 | CB | LYS | C | 59 | 11.742 | 39.483 | 20.270 | 1.00 | 248.43 |
| 439 | CG | LYS | C | 59 | 10.375 | 39.002 | 19.795 | 1.00 | 248.43 |
| 440 | CD | LYS | C | 59 | 9.436 | 40.157 | 19.482 | 1.00 | 248.43 |
| 441 | CE | LYS | C | 59 | 8.053 | 39.641 | 19.094 | 1.00 | 248.43 |
| 442 | NZ | LYS | C | 59 | 7.100 | 40.738 | 18.771 | 1.00 | 248.43 |
| 443 | C | LYS | C | 59 | 13.986 | 38.928 | 21.228 | 1.00 | 135.91 |
| 444 | O | LYS | C | 59 | 14.052 | 39.415 | 22.354 | 1.00 | 135.91 |
| 445 | N | PHE | C | 60 | 15.020 | 38.866 | 20.406 | 1.00 | 130.99 |
| 446 | CA | PHE | C | 60 | 16.330 | 39.375 | 20.784 | 1.00 | 130.99 |
| 447 | CB | PHE | C | 60 | 17.171 | 39.581 | 19.523 | 1.00 | 226.68 |
| 448 | CG | PHE | C | 60 | 17.469 | 38.309 | 18.781 | 1.00 | 226.68 |
| 449 | CD1 | PHE | C | 60 | 17.704 | 38.327 | 17.410 | 1.00 | 226.68 |
| 450 | CD2 | PHE | C | 60 | 17.535 | 37.093 | 19.458 | 1.00 | 226.68 |
| 451 | CE1 | PHE | C | 60 | 17.998 | 37.156 | 16.724 | 1.00 | 226.68 |
| 452 | CE2 | PHE | C | 60 | 17.829 | 35.919 | 18.782 | 1.00 | 226.68 |
| 453 | CZ | PHE | C | 60 | 18.061 | 35.951 | 17.411 | 1.00 | 226.68 |
| 454 | C | PHE | C | 60 | 16.296 | 40.672 | 21.597 | 1.00 | 130.99 |
| 455 | O | PHE | C | 60 | 17.171 | 40.914 | 22.439 | 1.00 | 130.99 |
| 456 | N | GLU | C | 61 | 15.289 | 41.507 | 21.338 | 1.00 | 229.15 |
| 457 | CA | GLU | C | 61 | 15.136 | 42.789 | 22.028 | 1.00 | 229.15 |
| 458 | CB | GLU | C | 61 | 14.021 | 43.603 | 21.363 | 1.00 | 236.43 |
| 459 | CG | GLU | C | 61 | 14.258 | 43.926 | 19.878 | 1.00 | 236.43 |
| 460 | CD | GLU | C | 61 | 14.424 | 42.686 | 19.003 | 1.00 | 236.43 |
| 461 | OE1 | GLU | C | 61 | 13.554 | 41.786 | 19.062 | 1.00 | 236.43 |
| 462 | OE2 | GLU | C | 61 | 15.423 | 42.616 | 18.250 | 1.00 | 236.43 |
| 463 | C | GLU | C | 61 | 14.832 | 42.608 | 23.508 | 1.00 | 229.15 |
| 464 | O | GLU | C | 61 | 15.107 | 43.491 | 24.316 | 1.00 | 229.15 |
| 465 | N | ASP | C | 62 | 14.260 | 41.456 | 23.849 | 1.00 | 169.19 |
| 466 | CA | ASP | C | 62 | 13.926 | 41.142 | 25.233 | 1.00 | 169.19 |
| 467 | CB | ASP | C | 62 | 13.066 | 39.884 | 25.316 | 1.00 | 219.70 |
| 468 | CG | ASP | C | 62 | 11.857 | 39.951 | 24.419 | 1.00 | 219.70 |
| 469 | OD1 | ASP | C | 62 | 11.324 | 41.064 | 24.225 | 1.00 | 219.70 |
| 470 | OD2 | ASP | C | 62 | 11.430 | 38.891 | 23.919 | 1.00 | 219.70 |
| 471 | C | ASP | C | 62 | 15.184 | 40.932 | 26.066 | 1.00 | 169.19 |
| 472 | O | ASP | C | 62 | 15.152 | 41.049 | 27.289 | 1.00 | 169.19 |
| 473 | N | SER | C | 63 | 16.289 | 40.608 | 25.400 | 1.00 | 159.66 |
| 474 | CA | SER | C | 63 | 17.564 | 40.400 | 26.084 | 1.00 | 159.66 |
| 475 | CB | SER | C | 63 | 18.659 | 39.965 | 25.089 | 1.00 | 141.40 |
| 476 | OG | SER | C | 63 | 18.325 | 38.774 | 24.394 | 1.00 | 141.40 |
| 477 | C | SER | C | 63 | 17.962 | 41.730 | 26.714 | 1.00 | 159.66 |
| 478 | O | SER | C | 63 | 18.006 | 42.746 | 26.029 | 1.00 | 159.66 |
| 479 | N | GLY | C | 64 | 18.242 | 41.730 | 28.009 | 1.00 | 163.83 |
| 480 | CA | GLY | C | 64 | 18.620 | 42.974 | 28.641 | 1.00 | 163.83 |
| 481 | C | GLY | C | 64 | 18.666 | 42.973 | 30.154 | 1.00 | 163.83 |
| 482 | O | GLY | C | 64 | 18.652 | 41.917 | 30.792 | 1.00 | 163.83 |
| 483 | N | GLU | C | 65 | 18.719 | 44.181 | 30.713 | 1.00 | 155.20 |
| 484 | CA | GLU | C | 65 | 18.792 | 44.422 | 32.152 | 1.00 | 155.20 |
| 485 | CB | GLU | C | 65 | 19.859 | 45.482 | 32.390 | 1.00 | 246.28 |
| 486 | CG | GLU | C | 65 | 19.972 | 45.990 | 33.800 | 1.00 | 246.28 |
| 487 | CD | GLU | C | 65 | 20.739 | 47.294 | 33.859 | 1.00 | 246.28 |
| 488 | OE1 | GLU | C | 65 | 20.270 | 48.276 | 33.250 | 1.00 | 246.28 |
| 489 | OE2 | GLU | C | 65 | 21.806 | 47.341 | 34.506 | 1.00 | 246.28 |
| 490 | C | GLU | C | 65 | 17.444 | 44.883 | 32.727 | 1.00 | 155.20 |
| 491 | O | GLU | C | 65 | 16.907 | 45.897 | 32.306 | 1.00 | 155.20 |
| 492 | N | TYR | C | 66 | 16.899 | 44.149 | 33.692 | 1.00 | 218.21 |
| 493 | CA | TYR | C | 66 | 15.614 | 44.507 | 34.299 | 1.00 | 218.21 |
| 494 | CB | TYR | C | 66 | 14.600 | 43.380 | 34.131 | 1.00 | 195.34 |
| 495 | CG | TYR | C | 66 | 14.195 | 43.050 | 32.722 | 1.00 | 195.34 |
| 496 | CD1 | TYR | C | 66 | 15.027 | 42.319 | 31.889 | 1.00 | 195.34 |
| 497 | CE1 | TYR | C | 66 | 14.607 | 41.929 | 30.619 | 1.00 | 195.34 |
| 498 | CD2 | TYR | C | 66 | 12.934 | 43.398 | 32.250 | 1.00 | 195.34 |
| 499 | CE2 | TYR | C | 66 | 12.505 | 43.016 | 30.985 | 1.00 | 195.34 |
| 500 | CZ | TYR | C | 66 | 13.342 | 42.277 | 30.175 | 1.00 | 195.34 |
| 501 | OH | TYR | C | 66 | 12.896 | 41.868 | 28.938 | 1.00 | 195.34 |
| 502 | C | TYR | C | 66 | 15.691 | 44.815 | 35.795 | 1.00 | 218.21 |
| 503 | O | TYR | C | 66 | 16.721 | 44.584 | 36.431 | 1.00 | 218.21 |
| 504 | N | LYS | C | 67 | 14.577 | 45.311 | 36.350 | 1.00 | 178.65 |
| 505 | CA | LYS | C | 67 | 14.467 | 45.652 | 37.782 | 1.00 | 178.65 |
| 506 | CB | LYS | C | 67 | 15.471 | 46.748 | 38.152 | 1.00 | 172.69 |
| 507 | CG | LYS | C | 67 | 15.399 | 47.981 | 37.275 | 1.00 | 172.69 |
| 508 | CD | LYS | C | 67 | 16.474 | 48.976 | 37.663 | 1.00 | 172.69 |
| 509 | CE | LYS | C | 67 | 16.722 | 50.003 | 36.565 | 1.00 | 172.69 |
| 510 | NZ | LYS | C | 67 | 17.749 | 51.022 | 36.952 | 1.00 | 172.69 |
| 511 | C | LYS | C | 67 | 13.078 | 46.103 | 38.229 | 1.00 | 178.65 |
| 512 | O | LYS | C | 67 | 12.289 | 46.623 | 37.437 | 1.00 | 178.65 |
| 513 | N | CYS | C | 68 | 12.794 | 45.898 | 39.512 | 1.00 | 193.02 |
| 514 | CA | CYS | C | 68 | 11.523 | 46.307 | 40.083 | 1.00 | 193.02 |
| 515 | C | CYS | C | 68 | 11.724 | 47.110 | 41.369 | 1.00 | 193.02 |
| 516 | O | CYS | C | 68 | 12.709 | 46.929 | 42.091 | 1.00 | 193.02 |
| 517 | CB | CYS | C | 68 | 10.604 | 45.104 | 40.336 | 1.00 | 142.23 |
| 518 | SG | CYS | C | 68 | 11.079 | 43.935 | 41.620 | 1.00 | 142.23 |
| 519 | N | GLN | C | 69 | 10.780 | 48.008 | 41.636 | 1.00 | 226.79 |
| 520 | CA | GLN | C | 69 | 10.806 | 48.882 | 42.802 | 1.00 | 226.79 |
| 521 | CB | GLN | C | 69 | 11.511 | 50.191 | 42.437 | 1.00 | 248.82 |
| 522 | CG | GLN | C | 69 | 11.193 | 51.363 | 43.344 | 1.00 | 248.82 |
| 523 | CD | GLN | C | 69 | 11.804 | 52.664 | 42.851 | 1.00 | 248.82 |
| 524 | OE1 | GLN | C | 69 | 11.623 | 53.050 | 41.694 | 1.00 | 248.82 |
| 525 | NE2 | GLN | C | 69 | 12.526 | 53.352 | 43.730 | 1.00 | 248.82 |
| 526 | C | GLN | C | 69 | 9.370 | 49.163 | 43.221 | 1.00 | 226.79 |
| 527 | O | GLN | C | 69 | 8.470 | 49.208 | 42.382 | 1.00 | 226.79 |
| 528 | N | HIS | C | 70 | 9.149 | 49.349 | 44.515 | 1.00 | 241.71 |
| 529 | CA | HIS | C | 70 | 7.806 | 49.635 | 45.003 | 1.00 | 241.71 |
| 530 | CB | HIS | C | 70 | 7.524 | 48.852 | 46.292 | 1.00 | 246.85 |
| 531 | CG | HIS | C | 70 | 7.366 | 47.378 | 46.075 | 1.00 | 246.85 |
| 532 | CD2 | HIS | C | 70 | 7.971 | 46.319 | 46.666 | 1.00 | 246.85 |
| 533 | ND1 | HIS | C | 70 | 6.487 | 46.852 | 45.155 | 1.00 | 246.85 |
| 534 | CE1 | HIS | C | 70 | 6.556 | 45.530 | 45.184 | 1.00 | 246.85 |
| 535 | NE2 | HIS | C | 70 | 7.448 | 45.184 | 46.094 | 1.00 | 246.85 |
| 536 | C | HIS | C | 70 | 7.601 | 51.127 | 45.236 | 1.00 | 241.71 |
| 537 | O | HIS | C | 70 | 8.435 | 51.946 | 44.851 | 1.00 | 241.71 |
| 538 | N | GLN | C | 71 | 6.485 | 51.470 | 45.872 | 1.00 | 248.91 |
| 539 | CA | GLN | C | 71 | 6.139 | 52.861 | 46.161 | 1.00 | 248.91 |
| 540 | CB | GLN | C | 71 | 4.804 | 52.897 | 46.935 | 1.00 | 249.69 |
| 541 | CG | GLN | C | 71 | 4.049 | 54.235 | 46.919 | 1.00 | 249.69 |
| 542 | CD | GLN | C | 71 | 3.630 | 54.682 | 45.519 | 1.00 | 249.69 |
| 543 | OE1 | GLN | C | 71 | 3.071 | 53.904 | 44.741 | 1.00 | 249.69 |
| 544 | NE2 | GLN | C | 71 | 3.889 | 55.949 | 45.201 | 1.00 | 249.69 |
| 545 | C | GLN | C | 71 | 7.243 | 53.579 | 46.956 | 1.00 | 248.91 |
| 546 | O | GLN | C | 71 | 7.670 | 54.680 | 46.599 | 1.00 | 248.91 |
| 547 | N | GLN | C | 72 | 7.705 | 52.942 | 48.026 | 1.00 | 236.85 |
| 548 | CA | GLN | C | 72 | 8.741 | 53.519 | 48.875 | 1.00 | 236.85 |
| 549 | CB | GLN | C | 72 | 8.117 | 53.962 | 50.201 | 1.00 | 249.69 |
| 550 | CG | GLN | C | 72 | 9.064 | 54.624 | 51.198 | 1.00 | 249.69 |
| 551 | CD | GLN | C | 72 | 8.391 | 54.885 | 52.545 | 1.00 | 249.69 |
| 552 | OE1 | GLN | C | 72 | 7.360 | 55.560 | 52.617 | 1.00 | 249.69 |
| 553 | NE2 | GLN | C | 72 | 8.973 | 54.348 | 53.617 | 1.00 | 249.69 |
| 554 | C | GLN | C | 72 | 9.860 | 52.501 | 49.127 | 1.00 | 236.85 |
| 555 | O | GLN | C | 72 | 10.188 | 52.188 | 50.274 | 1.00 | 236.85 |
| 556 | N | VAL | C | 73 | 10.435 | 51.975 | 48.050 | 1.00 | 249.69 |
| 557 | CA | VAL | C | 73 | 11.519 | 51.001 | 48.163 | 1.00 | 249.69 |
| 558 | CB | VAL | C | 73 | 11.016 | 49.546 | 47.988 | 1.00 | 190.27 |
| 559 | CG1 | VAL | C | 73 | 12.100 | 48.583 | 48.424 | 1.00 | 190.27 |
| 560 | CG2 | VAL | C | 73 | 9.740 | 49.315 | 48.789 | 1.00 | 190.27 |
| 561 | C | VAL | C | 73 | 12.547 | 51.280 | 47.077 | 1.00 | 249.69 |
| 562 | O | VAL | C | 73 | 12.195 | 51.674 | 45.966 | 1.00 | 249.69 |
| 563 | N | ASN | C | 74 | 13.819 | 51.080 | 47.397 | 1.00 | 225.53 |
| 564 | CA | ASN | C | 74 | 14.877 | 51.314 | 46.426 | 1.00 | 225.53 |
| 565 | CB | ASN | C | 74 | 16.220 | 51.480 | 47.153 | 1.00 | 240.44 |
| 566 | CG | ASN | C | 74 | 16.174 | 52.577 | 48.221 | 1.00 | 240.44 |
| 567 | OD1 | ASN | C | 74 | 15.597 | 53.643 | 47.999 | 1.00 | 240.44 |
| 568 | ND2 | ASN | C | 74 | 16.786 | 52.316 | 49.374 | 1.00 | 240.44 |
| 569 | C | ASN | C | 74 | 14.929 | 50.162 | 45.407 | 1.00 | 225.53 |
| 570 | O | ASN | C | 74 | 14.963 | 48.987 | 45.778 | 1.00 | 225.53 |
| 571 | N | GLU | C | 75 | 14.918 | 50.516 | 44.123 | 1.00 | 249.69 |
| 572 | CA | GLU | C | 75 | 14.943 | 49.543 | 43.030 | 1.00 | 249.69 |
| 573 | CB | GLU | C | 75 | 15.262 | 50.260 | 41.708 | 1.00 | 249.60 |
| 574 | CG | GLU | C | 75 | 16.260 | 51.404 | 41.834 | 1.00 | 249.60 |
| 575 | CD | GLU | C | 75 | 16.362 | 52.238 | 40.567 | 1.00 | 249.60 |
| 576 | OE1 | GLU | C | 75 | 15.308 | 52.660 | 40.046 | 1.00 | 249.60 |
| 577 | OE2 | GLU | C | 75 | 17.493 | 52.480 | 40.096 | 1.00 | 249.60 |
| 578 | C | GLU | C | 75 | 15.886 | 48.356 | 43.233 | 1.00 | 249.69 |
| 579 | O | GLU | C | 75 | 16.998 | 48.508 | 43.733 | 1.00 | 249.69 |
| 580 | N | SER | C | 76 | 15.421 | 47.175 | 42.830 | 1.00 | 230.56 |
| 581 | CA | SER | C | 76 | 16.178 | 45.932 | 42.968 | 1.00 | 230.56 |
| 582 | CB | SER | C | 76 | 15.307 | 44.734 | 42.596 | 1.00 | 187.63 |
| 583 | OG | SER | C | 76 | 15.123 | 44.661 | 41.190 | 1.00 | 187.63 |
| 584 | C | SER | C | 76 | 17.435 | 45.877 | 42.115 | 1.00 | 230.56 |
| 585 | O | SER | C | 76 | 17.565 | 46.600 | 41.124 | 1.00 | 230.56 |
| 586 | N | GLU | C | 77 | 18.356 | 45.002 | 42.510 | 1.00 | 249.20 |
| 587 | CA | GLU | C | 77 | 19.602 | 44.825 | 41.778 | 1.00 | 249.20 |
| 588 | CB | GLU | C | 77 | 20.531 | 43.861 | 42.527 | 1.00 | 249.69 |
| 589 | CG | GLU | C | 77 | 21.030 | 44.390 | 43.870 | 1.00 | 249.69 |
| 590 | CD | GLU | C | 77 | 21.895 | 45.639 | 43.734 | 1.00 | 249.69 |
| 591 | OE1 | GLU | C | 77 | 22.002 | 46.187 | 42.611 | 1.00 | 249.69 |
| 592 | OE2 | GLU | C | 77 | 22.468 | 46.078 | 44.755 | 1.00 | 249.69 |
| 593 | C | GLU | C | 77 | 19.257 | 44.256 | 40.410 | 1.00 | 249.20 |
| 594 | O | GLU | C | 77 | 18.786 | 43.124 | 40.304 | 1.00 | 249.20 |
| 595 | N | PRO | C | 78 | 19.492 | 45.042 | 39.346 | 1.00 | 211.01 |
| 596 | CD | PRO | C | 78 | 20.275 | 46.292 | 39.342 | 1.00 | 171.69 |
| 597 | CA | PRO | C | 78 | 19.193 | 44.608 | 37.977 | 1.00 | 211.01 |
| 598 | CB | PRO | C | 78 | 20.023 | 45.581 | 37.127 | 1.00 | 171.69 |
| 599 | CG | PRO | C | 78 | 20.054 | 46.826 | 37.951 | 1.00 | 171.69 |
| 600 | C | PRO | C | 78 | 19.580 | 43.155 | 37.720 | 1.00 | 211.01 |
| 601 | O | PRO | C | 78 | 20.416 | 42.597 | 38.420 | 1.00 | 211.01 |
| 602 | N | VAL | C | 79 | 18.950 | 42.537 | 36.728 | 1.00 | 200.35 |
| 603 | CA | VAL | C | 79 | 19.282 | 41.166 | 36.344 | 1.00 | 200.35 |
| 604 | CB | VAL | C | 79 | 18.203 | 40.146 | 36.754 | 1.00 | 129.43 |
| 605 | CG1 | VAL | C | 79 | 18.471 | 38.805 | 36.090 | 1.00 | 129.43 |
| 606 | CG2 | VAL | C | 79 | 18.208 | 39.966 | 38.259 | 1.00 | 129.43 |
| 607 | C | VAL | C | 79 | 19.371 | 41.203 | 34.835 | 1.00 | 200.35 |
| 608 | O | VAL | C | 79 | 18.589 | 41.902 | 34.191 | 1.00 | 200.35 |
| 609 | N | TYR | C | 80 | 20.324 | 40.476 | 34.262 | 1.00 | 130.20 |
| 610 | CA | TYR | C | 80 | 20.458 | 40.495 | 32.817 | 1.00 | 130.20 |
| 611 | CB | TYR | C | 80 | 21.910 | 40.686 | 32.410 | 1.00 | 206.89 |
| 612 | CG | TYR | C | 80 | 22.046 | 41.126 | 30.971 | 1.00 | 206.89 |
| 613 | CD1 | TYR | C | 80 | 21.927 | 42.471 | 30.618 | 1.00 | 206.89 |
| 614 | CE1 | TYR | C | 80 | 22.011 | 42.880 | 29.290 | 1.00 | 206.89 |
| 615 | CD2 | TYR | C | 80 | 22.254 | 40.199 | 29.954 | 1.00 | 206.89 |
| 616 | CE2 | TYR | C | 80 | 22.341 | 40.598 | 28.621 | 1.00 | 206.89 |
| 617 | CZ | TYR | C | 80 | 22.217 | 41.939 | 28.299 | 1.00 | 206.89 |
| 618 | OH | TYR | C | 80 | 22.299 | 42.339 | 26.988 | 1.00 | 206.89 |
| 619 | C | TYR | C | 80 | 19.928 | 39.237 | 32.170 | 1.00 | 130.20 |
| 620 | O | TYR | C | 80 | 20.195 | 38.133 | 32.638 | 1.00 | 130.20 |
| 621 | N | LEU | C | 81 | 19.172 | 39.406 | 31.093 | 1.00 | 124.74 |
| 622 | CA | LEU | C | 81 | 18.624 | 38.266 | 30.390 | 1.00 | 124.74 |
| 623 | CB | LEU | C | 81 | 17.103 | 38.367 | 30.285 | 1.00 | 90.50 |
| 624 | CG | LEU | C | 81 | 16.470 | 37.260 | 29.428 | 1.00 | 90.50 |
| 625 | CD1 | LEU | C | 81 | 16.710 | 35.944 | 30.122 | 1.00 | 90.50 |
| 626 | CD2 | LEU | C | 81 | 14.994 | 37.467 | 29.222 | 1.00 | 90.50 |
| 627 | C | LEU | C | 81 | 19.216 | 38.222 | 28.998 | 1.00 | 124.74 |
| 628 | O | LEU | C | 81 | 19.179 | 39.232 | 28.300 | 1.00 | 124.74 |
| 629 | N | GLU | C | 82 | 19.771 | 37.075 | 28.595 | 1.00 | 106.68 |
| 630 | CA | GLU | C | 82 | 20.322 | 36.967 | 27.253 | 1.00 | 106.68 |
| 631 | CB | GLU | C | 82 | 21.797 | 36.601 | 27.305 | 1.00 | 249.60 |
| 632 | CG | GLU | C | 82 | 22.564 | 37.080 | 26.079 | 1.00 | 249.60 |
| 633 | CD | GLU | C | 82 | 24.041 | 36.748 | 26.143 | 1.00 | 249.60 |
| 634 | OE1 | GLU | C | 82 | 24.609 | 36.761 | 27.257 | 1.00 | 249.60 |
| 635 | OE2 | GLU | C | 82 | 24.637 | 36.489 | 25.075 | 1.00 | 249.60 |
| 636 | C | GLU | C | 82 | 19.546 | 35.917 | 26.454 | 1.00 | 106.68 |
| 637 | O | GLU | C | 82 | 19.224 | 34.834 | 26.994 | 1.00 | 106.68 |
| 638 | N | VAL | C | 83 | 19.234 | 36.244 | 25.186 | 1.00 | 145.59 |
| 639 | CA | VAL | C | 83 | 18.513 | 35.318 | 24.292 | 1.00 | 145.59 |
| 640 | CB | VAL | C | 83 | 17.270 | 35.943 | 23.679 | 1.00 | 134.02 |
| 641 | CG1 | VAL | C | 83 | 16.562 | 34.914 | 22.796 | 1.00 | 134.02 |
| 642 | CG2 | VAL | C | 83 | 16.343 | 36.429 | 24.781 | 1.00 | 134.02 |
| 643 | C | VAL | C | 83 | 19.417 | 34.835 | 23.164 | 1.00 | 145.59 |
| 644 | O | VAL | C | 83 | 20.212 | 35.603 | 22.600 | 1.00 | 145.59 |
| 645 | N | PHE | C | 84 | 19.259 | 33.562 | 22.822 | 1.00 | 150.61 |
| 646 | CA | PHE | C | 84 | 20.117 | 32.945 | 21.833 | 1.00 | 150.61 |
| 647 | CB | PHE | C | 84 | 21.072 | 31.978 | 22.515 | 1.00 | 134.04 |
| 648 | CG | PHE | C | 84 | 21.985 | 32.603 | 23.516 | 1.00 | 134.04 |
| 649 | CD1 | PHE | C | 84 | 21.566 | 32.852 | 24.818 | 1.00 | 134.04 |
| 650 | CD2 | PHE | C | 84 | 23.282 | 32.918 | 23.158 | 1.00 | 134.04 |
| 651 | CE1 | PHE | C | 84 | 22.433 | 33.403 | 25.743 | 1.00 | 134.04 |
| 652 | CE2 | PHE | C | 84 | 24.151 | 33.468 | 24.078 | 1.00 | 134.04 |
| 653 | CZ | PHE | C | 84 | 23.729 | 33.712 | 25.370 | 1.00 | 134.04 |
| 654 | C | PHE | C | 84 | 19.487 | 32.151 | 20.719 | 1.00 | 150.61 |
| 655 | O | PHE | C | 84 | 18.363 | 31.654 | 20.842 | 1.00 | 150.61 |
| 656 | N | SER | C | 85 | 20.276 | 31.999 | 19.653 | 1.00 | 176.12 |
| 657 | CA | SER | C | 85 | 19.898 | 31.208 | 18.491 | 1.00 | 176.12 |
| 658 | CB | SER | C | 85 | 19.635 | 32.073 | 17.269 | 1.00 | 141.64 |
| 659 | OG | SER | C | 85 | 19.275 | 31.250 | 16.175 | 1.00 | 141.64 |
| 660 | C | SER | C | 85 | 21.092 | 30.334 | 18.214 | 1.00 | 176.12 |
| 661 | O | SER | C | 85 | 22.171 | 30.838 | 17.876 | 1.00 | 176.12 |
| 662 | N | ASP | C | 86 | 20.900 | 29.030 | 18.377 | 1.00 | 126.17 |
| 663 | CA | ASP | C | 86 | 21.976 | 28.062 | 18.156 | 1.00 | 126.17 |
| 664 | CB | ASP | C | 86 | 23.135 | 28.325 | 19.122 | 1.00 | 148.28 |
| 665 | CG | ASP | C | 86 | 24.477 | 28.085 | 18.490 | 1.00 | 148.28 |
| 666 | OD1 | ASP | C | 86 | 24.674 | 26.995 | 17.898 | 1.00 | 148.28 |
| 667 | OD2 | ASP | C | 86 | 25.329 | 28.999 | 18.587 | 1.00 | 148.28 |
| 668 | C | ASP | C | 86 | 21.448 | 26.656 | 18.392 | 1.00 | 126.17 |
| 669 | O | ASP | C | 86 | 20.356 | 26.480 | 18.922 | 1.00 | 126.17 |
| 670 | N | TRP | C | 87 | 22.220 | 25.650 | 18.003 | 1.00 | 154.85 |
| 671 | CA | TRP | C | 87 | 21.780 | 24.277 | 18.204 | 1.00 | 154.85 |
| 672 | CB | TRP | C | 87 | 22.714 | 23.312 | 17.473 | 1.00 | 249.47 |
| 673 | CG | TRP | C | 87 | 22.275 | 23.058 | 16.067 | 1.00 | 249.47 |
| 674 | CD2 | TRP | C | 87 | 22.713 | 23.754 | 14.895 | 1.00 | 249.47 |
| 675 | CE2 | TRP | C | 87 | 22.000 | 23.217 | 13.798 | 1.00 | 249.47 |
| 676 | CE3 | TRP | C | 87 | 23.645 | 24.782 | 14.661 | 1.00 | 249.47 |
| 677 | CD1 | TRP | C | 87 | 21.335 | 22.152 | 15.650 | 1.00 | 249.47 |
| 678 | NE1 | TRP | C | 87 | 21.165 | 22.243 | 14.290 | 1.00 | 249.47 |
| 679 | CZ2 | TRP | C | 87 | 22.184 | 23.670 | 12.489 | 1.00 | 249.47 |
| 680 | CZ3 | TRP | C | 87 | 23.828 | 25.232 | 13.361 | 1.00 | 249.47 |
| 681 | CH2 | TRP | C | 87 | 23.098 | 24.675 | 12.291 | 1.00 | 249.47 |
| 682 | C | TRP | C | 87 | 21.715 | 23.947 | 19.683 | 1.00 | 154.85 |
| 683 | O | TRP | C | 87 | 20.676 | 23.513 | 20.170 | 1.00 | 154.85 |
| 684 | N | LEU | C | 88 | 22.820 | 24.160 | 20.394 | 1.00 | 160.72 |
| 685 | CA | LEU | C | 88 | 22.851 | 23.888 | 21.820 | 1.00 | 160.72 |
| 686 | CB | LEU | C | 88 | 23.811 | 22.726 | 22.122 | 1.00 | 161.67 |
| 687 | CG | LEU | C | 88 | 23.421 | 21.360 | 21.543 | 1.00 | 161.67 |
| 688 | CD1 | LEU | C | 88 | 24.392 | 20.299 | 22.027 | 1.00 | 161.67 |
| 689 | CD2 | LEU | C | 88 | 22.005 | 21.001 | 21.965 | 1.00 | 161.67 |
| 690 | C | LEU | C | 88 | 23.251 | 25.124 | 22.623 | 1.00 | 160.72 |
| 691 | O | LEU | C | 88 | 24.103 | 25.909 | 22.192 | 1.00 | 160.72 |
| 692 | N | LEU | C | 89 | 22.613 | 25.306 | 23.780 | 1.00 | 139.08 |
| 693 | CA | LEU | C | 89 | 22.929 | 26.426 | 24.654 | 1.00 | 139.08 |
| 694 | CB | LEU | C | 89 | 21.801 | 27.440 | 24.663 | 1.00 | 166.24 |
| 695 | CG | LEU | C | 89 | 22.043 | 28.607 | 25.629 | 1.00 | 166.24 |
| 696 | CD1 | LEU | C | 89 | 23.405 | 29.250 | 25.356 | 1.00 | 166.24 |
| 697 | CD2 | LEU | C | 89 | 20.917 | 29.623 | 25.492 | 1.00 | 166.24 |
| 698 | C | LEU | C | 89 | 23.123 | 25.891 | 26.057 | 1.00 | 139.08 |
| 699 | O | LEU | C | 89 | 22.297 | 25.098 | 26.533 | 1.00 | 139.08 |
| 700 | N | LEU | C | 90 | 24.212 | 26.304 | 26.715 | 1.00 | 149.33 |
| 701 | CA | LEU | C | 90 | 24.490 | 25.844 | 28.077 | 1.00 | 149.33 |
| 702 | CB | LEU | C | 90 | 25.993 | 25.806 | 28.323 | 1.00 | 143.04 |
| 703 | CG | LEU | C | 90 | 26.370 | 25.474 | 29.765 | 1.00 | 143.04 |
| 704 | CD1 | LEU | C | 90 | 25.808 | 24.104 | 30.144 | 1.00 | 143.04 |
| 705 | CD2 | LEU | C | 90 | 27.884 | 25.509 | 29.938 | 1.00 | 143.04 |
| 706 | C | LEU | C | 90 | 23.834 | 26.755 | 29.106 | 1.00 | 149.33 |
| 707 | O | LEU | C | 90 | 24.213 | 27.914 | 29.243 | 1.00 | 149.33 |
| 708 | N | GLN | C | 91 | 22.861 | 26.226 | 29.839 | 1.00 | 125.14 |
| 709 | CA | GLN | C | 91 | 22.166 | 27.026 | 30.825 | 1.00 | 125.14 |
| 710 | CB | GLN | C | 91 | 20.656 | 26.784 | 30.745 | 1.00 | 164.13 |
| 711 | CG | GLN | C | 91 | 20.043 | 27.113 | 29.398 | 1.00 | 164.13 |
| 712 | CD | GLN | C | 91 | 18.552 | 26.873 | 29.373 | 1.00 | 164.13 |
| 713 | OE1 | GLN | C | 91 | 18.078 | 25.769 | 29.661 | 1.00 | 164.13 |
| 714 | NE2 | GLN | C | 91 | 17.799 | 27.910 | 29.035 | 1.00 | 164.13 |
| 715 | C | GLN | C | 91 | 22.633 | 26.746 | 32.238 | 1.00 | 125.14 |
| 716 | O | GLN | C | 91 | 22.832 | 25.583 | 32.625 | 1.00 | 125.14 |
| 717 | N | ALA | C | 92 | 22.787 | 27.820 | 33.014 | 1.00 | 120.03 |
| 718 | CA | ALA | C | 92 | 23.217 | 27.706 | 34.404 | 1.00 | 120.03 |
| 719 | CB | ALA | C | 92 | 24.586 | 28.363 | 34.567 | 1.00 | 230.41 |
| 720 | C | ALA | C | 92 | 22.204 | 28.360 | 35.331 | 1.00 | 120.03 |
| 721 | O | ALA | C | 92 | 21.618 | 29.392 | 34.993 | 1.00 | 120.03 |
| 722 | N | SER | C | 93 | 22.009 | 27.738 | 36.490 | 1.00 | 162.82 |
| 723 | CA | SER | C | 93 | 21.091 | 28.244 | 37.499 | 1.00 | 162.82 |
| 724 | CB | SER | C | 93 | 21.158 | 27.396 | 38.784 | 1.00 | 102.92 |
| 725 | OG | SER | C | 93 | 22.476 | 27.269 | 39.270 | 1.00 | 102.92 |
| 726 | C | SER | C | 93 | 21.472 | 29.682 | 37.798 | 1.00 | 162.82 |
| 727 | O | SER | C | 93 | 20.699 | 30.618 | 37.567 | 1.00 | 162.82 |
| 728 | N | ALA | C | 94 | 22.679 | 29.849 | 38.313 | 1.00 | 108.42 |
| 729 | CA | ALA | C | 94 | 23.224 | 31.174 | 38.620 | 1.00 | 108.42 |
| 730 | CB | ALA | C | 94 | 23.252 | 31.403 | 40.121 | 1.00 | 218.96 |
| 731 | C | ALA | C | 94 | 24.643 | 31.150 | 38.051 | 1.00 | 108.42 |
| 732 | O | ALA | C | 94 | 25.237 | 30.083 | 37.932 | 1.00 | 108.42 |
| 733 | N | GLU | C | 95 | 25.180 | 32.303 | 37.678 | 1.00 | 153.28 |
| 734 | CA | GLU | C | 95 | 26.518 | 32.317 | 37.122 | 1.00 | 153.28 |
| 735 | CB | GLU | C | 95 | 26.615 | 33.364 | 36.025 | 1.00 | 202.07 |
| 736 | CG | GLU | C | 95 | 25.708 | 33.060 | 34.858 | 1.00 | 202.07 |
| 737 | CD | GLU | C | 95 | 25.982 | 33.949 | 33.677 | 1.00 | 202.07 |
| 738 | OE1 | GLU | C | 95 | 25.257 | 33.821 | 32.668 | 1.00 | 202.07 |
| 739 | OE2 | GLU | C | 95 | 26.925 | 34.772 | 33.751 | 1.00 | 202.07 |
| 740 | C | GLU | C | 95 | 27.586 | 32.559 | 38.176 | 1.00 | 153.28 |
| 741 | O | GLU | C | 95 | 28.757 | 32.209 | 37.973 | 1.00 | 153.28 |
| 742 | N | VAL | C | 96 | 27.180 | 33.151 | 39.302 | 1.00 | 129.17 |
| 743 | CA | VAL | C | 96 | 28.105 | 33.428 | 40.407 | 1.00 | 129.17 |
| 744 | CB | VAL | C | 96 | 28.289 | 34.930 | 40.613 | 1.00 | 121.01 |
| 745 | CG1 | VAL | C | 96 | 29.526 | 35.175 | 41.441 | 1.00 | 121.01 |
| 746 | CG2 | VAL | C | 96 | 28.379 | 35.630 | 39.273 | 1.00 | 121.01 |
| 747 | C | VAL | C | 96 | 27.548 | 32.826 | 41.694 | 1.00 | 129.17 |
| 748 | O | VAL | C | 96 | 26.380 | 33.012 | 42.009 | 1.00 | 129.17 |
| 749 | N | VAL | C | 97 | 28.383 | 32.123 | 42.449 | 1.00 | 144.84 |
| 750 | CA | VAL | C | 97 | 27.885 | 31.495 | 43.658 | 1.00 | 144.84 |
| 751 | CB | VAL | C | 97 | 27.584 | 30.011 | 43.391 | 1.00 | 123.55 |
| 752 | CG1 | VAL | C | 97 | 26.631 | 29.492 | 44.443 | 1.00 | 123.55 |
| 753 | CG2 | VAL | C | 97 | 27.013 | 29.816 | 42.007 | 1.00 | 123.55 |
| 754 | C | VAL | C | 97 | 28.756 | 31.574 | 44.921 | 1.00 | 144.84 |
| 755 | O | VAL | C | 97 | 29.987 | 31.649 | 44.847 | 1.00 | 144.84 |
| 756 | N | MET | C | 98 | 28.083 | 31.537 | 46.073 | 1.00 | 143.10 |
| 757 | CA | MET | C | 98 | 28.713 | 31.568 | 47.396 | 1.00 | 143.10 |
| 758 | CB | MET | C | 98 | 27.725 | 32.094 | 48.440 | 1.00 | 249.69 |
| 759 | CG | MET | C | 98 | 27.288 | 33.530 | 48.256 | 1.00 | 249.69 |
| 760 | SD | MET | C | 98 | 28.558 | 34.688 | 48.776 | 1.00 | 249.69 |
| 761 | CE | MET | C | 98 | 28.400 | 34.595 | 50.561 | 1.00 | 249.69 |
| 762 | C | MET | C | 98 | 29.093 | 30.140 | 47.792 | 1.00 | 143.10 |
| 763 | O | MET | C | 98 | 28.224 | 29.260 | 47.820 | 1.00 | 143.10 |
| 764 | N | GLU | C | 99 | 30.366 | 29.905 | 48.115 | 1.00 | 134.64 |
| 765 | CA | GLU | C | 99 | 30.817 | 28.560 | 48.495 | 1.00 | 134.64 |
| 766 | CB | GLU | C | 99 | 32.113 | 28.640 | 49.296 | 1.00 | 249.69 |
| 767 | CG | GLU | C | 99 | 32.954 | 27.373 | 49.225 | 1.00 | 249.69 |
| 768 | CD | GLU | C | 99 | 34.077 | 27.361 | 50.242 | 1.00 | 249.69 |
| 769 | OE1 | GLU | C | 99 | 34.676 | 28.433 | 50.488 | 1.00 | 249.69 |
| 770 | OE2 | GLU | C | 99 | 34.370 | 26.275 | 50.787 | 1.00 | 249.69 |
| 771 | C | GLU | C | 99 | 29.760 | 27.848 | 49.328 | 1.00 | 134.64 |
| 772 | O | GLU | C | 99 | 29.307 | 28.382 | 50.333 | 1.00 | 134.64 |
| 773 | N | GLY | C | 100 | 29.348 | 26.660 | 48.899 | 1.00 | 174.64 |
| 774 | CA | GLY | C | 100 | 28.349 | 25.925 | 49.654 | 1.00 | 174.64 |
| 775 | C | GLY | C | 100 | 26.950 | 25.885 | 49.070 | 1.00 | 174.64 |
| 776 | O | GLY | C | 100 | 26.164 | 25.012 | 49.424 | 1.00 | 174.64 |
| 777 | N | GLN | C | 101 | 26.630 | 26.825 | 48.185 | 1.00 | 145.91 |
| 778 | CA | GLN | C | 101 | 25.309 | 26.876 | 47.562 | 1.00 | 145.91 |
| 779 | CB | GLN | C | 101 | 25.060 | 28.258 | 46.960 | 1.00 | 202.42 |
| 780 | CG | GLN | C | 101 | 24.842 | 29.331 | 47.995 | 1.00 | 202.42 |
| 781 | CD | GLN | C | 101 | 23.913 | 28.865 | 49.091 | 1.00 | 202.42 |
| 782 | OE1 | GLN | C | 101 | 24.266 | 27.997 | 49.893 | 1.00 | 202.42 |
| 783 | NE2 | GLN | C | 101 | 22.712 | 29.428 | 49.123 | 1.00 | 202.42 |
| 784 | C | GLN | C | 101 | 25.106 | 25.805 | 46.487 | 1.00 | 145.91 |
| 785 | O | GLN | C | 101 | 26.031 | 25.063 | 46.149 | 1.00 | 145.91 |
| 786 | N | PRO | C | 102 | 23.886 | 25.701 | 45.939 | 1.00 | 126.29 |
| 787 | CD | PRO | C | 102 | 22.626 | 26.310 | 46.410 | 1.00 | 226.98 |
| 788 | CA | PRO | C | 102 | 23.621 | 24.698 | 44.908 | 1.00 | 126.29 |
| 789 | CB | PRO | C | 102 | 22.151 | 24.385 | 45.124 | 1.00 | 226.98 |
| 790 | CG | PRO | C | 102 | 21.598 | 25.737 | 45.443 | 1.00 | 226.98 |
| 791 | C | PRO | C | 102 | 23.911 | 25.213 | 43.497 | 1.00 | 126.29 |
| 792 | O | PRO | C | 102 | 23.787 | 26.412 | 43.199 | 1.00 | 126.29 |
| 793 | N | LEU | C | 103 | 24.286 | 24.291 | 42.620 | 1.00 | 131.06 |
| 794 | CA | LEU | C | 103 | 24.592 | 24.644 | 41.240 | 1.00 | 131.06 |
| 795 | CB | LEU | C | 103 | 26.086 | 24.692 | 41.058 | 1.00 | 130.31 |
| 796 | CG | LEU | C | 103 | 26.385 | 25.294 | 39.703 | 1.00 | 130.31 |
| 797 | CD1 | LEU | C | 103 | 25.983 | 26.756 | 39.788 | 1.00 | 130.31 |
| 798 | CD2 | LEU | C | 103 | 27.857 | 25.143 | 39.332 | 1.00 | 130.31 |
| 799 | C | LEU | C | 103 | 24.020 | 23.658 | 40.214 | 1.00 | 131.06 |
| 800 | O | LEU | C | 103 | 24.265 | 22.458 | 40.297 | 1.00 | 131.06 |
| 801 | N | PHE | C | 104 | 23.267 | 24.151 | 39.239 | 1.00 | 115.91 |
| 802 | CA | PHE | C | 104 | 22.698 | 23.265 | 38.229 | 1.00 | 115.91 |
| 803 | CB | PHE | C | 104 | 21.177 | 23.174 | 38.354 | 1.00 | 184.63 |
| 804 | CG | PHE | C | 104 | 20.701 | 22.781 | 39.706 | 1.00 | 184.63 |
| 805 | CD1 | PHE | C | 104 | 20.673 | 23.704 | 40.748 | 1.00 | 184.63 |
| 806 | CD2 | PHE | C | 104 | 20.284 | 21.483 | 39.950 | 1.00 | 184.63 |
| 807 | CE1 | PHE | C | 104 | 20.232 | 23.335 | 42.025 | 1.00 | 184.63 |
| 808 | CE2 | PHE | C | 104 | 19.841 | 21.104 | 41.225 | 1.00 | 184.63 |
| 809 | CZ | PHE | C | 104 | 19.815 | 22.034 | 42.264 | 1.00 | 184.63 |
| 810 | C | PHE | C | 104 | 23.026 | 23.754 | 36.826 | 1.00 | 115.91 |
| 811 | O | PHE | C | 104 | 22.731 | 24.898 | 36.464 | 1.00 | 115.91 |
| 812 | N | LEU | C | 105 | 23.636 | 22.890 | 36.025 | 1.00 | 135.47 |
| 813 | CA | LEU | C | 105 | 23.955 | 23.247 | 34.643 | 1.00 | 135.47 |
| 814 | CB | LEU | C | 105 | 25.417 | 23.009 | 34.331 | 1.00 | 111.96 |
| 815 | CG | LEU | C | 105 | 26.347 | 23.800 | 35.242 | 1.00 | 111.96 |
| 816 | CD1 | LEU | C | 105 | 27.796 | 23.589 | 34.767 | 1.00 | 111.96 |
| 817 | CD2 | LEU | C | 105 | 25.961 | 25.269 | 35.233 | 1.00 | 111.96 |
| 818 | C | LEU | C | 105 | 23.101 | 22.381 | 33.740 | 1.00 | 135.47 |
| 819 | O | LEU | C | 105 | 22.734 | 21.264 | 34.094 | 1.00 | 135.47 |
| 820 | N | ARG | C | 106 | 22.782 | 22.888 | 32.564 | 1.00 | 142.54 |
| 821 | CA | ARG | C | 106 | 21.928 | 22.134 | 31.679 | 1.00 | 142.54 |
| 822 | CB | ARG | C | 106 | 20.500 | 22.619 | 31.876 | 1.00 | 187.32 |
| 823 | CG | ARG | C | 106 | 19.479 | 21.927 | 31.044 | 1.00 | 187.32 |
| 824 | CD | ARG | C | 106 | 18.129 | 22.598 | 31.190 | 1.00 | 187.32 |
| 825 | NE | ARG | C | 106 | 17.177 | 22.007 | 30.262 | 1.00 | 187.32 |
| 826 | CZ | ARG | C | 106 | 16.158 | 22.658 | 29.719 | 1.00 | 187.32 |
| 827 | NH1 | ARG | C | 106 | 15.956 | 23.937 | 30.020 | 1.00 | 187.32 |
| 828 | NH2 | ARG | C | 106 | 15.359 | 22.032 | 28.858 | 1.00 | 187.32 |
| 829 | C | ARG | C | 106 | 22.347 | 22.297 | 30.232 | 1.00 | 142.54 |
| 830 | O | ARG | C | 106 | 22.485 | 23.424 | 29.750 | 1.00 | 142.54 |
| 831 | N | CYS | C | 107 | 22.580 | 21.177 | 29.547 | 1.00 | 145.66 |
| 832 | CA | CYS | C | 107 | 22.950 | 21.221 | 28.129 | 1.00 | 145.66 |
| 833 | C | CYS | C | 107 | 21.612 | 21.282 | 27.439 | 1.00 | 145.66 |
| 834 | O | CYS | C | 107 | 20.923 | 20.257 | 27.350 | 1.00 | 145.66 |
| 835 | CB | CYS | C | 107 | 23.679 | 19.945 | 27.717 | 1.00 | 147.17 |
| 836 | SG | CYS | C | 107 | 24.521 | 20.003 | 26.086 | 1.00 | 147.17 |
| 837 | N | HIS | C | 108 | 21.242 | 22.477 | 26.975 | 1.00 | 187.51 |
| 838 | CA | HIS | C | 108 | 19.945 | 22.697 | 26.334 | 1.00 | 187.51 |
| 839 | CB | HIS | C | 108 | 19.369 | 24.051 | 26.763 | 1.00 | 249.50 |
| 840 | CG | HIS | C | 108 | 17.934 | 24.251 | 26.389 | 1.00 | 249.50 |
| 841 | CD2 | HIS | C | 108 | 17.311 | 25.271 | 25.756 | 1.00 | 249.50 |
| 842 | ND1 | HIS | C | 108 | 16.945 | 23.348 | 26.724 | 1.00 | 249.50 |
| 843 | CE1 | HIS | C | 108 | 15.777 | 23.808 | 26.317 | 1.00 | 249.50 |
| 844 | NE2 | HIS | C | 108 | 15.969 | 24.974 | 25.727 | 1.00 | 249.50 |
| 845 | C | HIS | C | 108 | 19.929 | 22.622 | 24.824 | 1.00 | 187.51 |
| 846 | O | HIS | C | 108 | 20.677 | 23.334 | 24.148 | 1.00 | 187.51 |
| 847 | N | GLY | C | 109 | 19.049 | 21.765 | 24.310 | 1.00 | 207.18 |
| 848 | CA | GLY | C | 109 | 18.916 | 21.601 | 22.879 | 1.00 | 207.18 |
| 849 | C | GLY | C | 109 | 17.989 | 22.668 | 22.337 | 1.00 | 207.18 |
| 850 | O | GLY | C | 109 | 17.304 | 23.341 | 23.106 | 1.00 | 207.18 |
| 851 | N | TRP | C | 110 | 17.976 | 22.837 | 21.017 | 1.00 | 133.19 |
| 852 | CA | TRP | C | 110 | 17.119 | 23.828 | 20.384 | 1.00 | 133.19 |
| 853 | CB | TRP | C | 110 | 17.724 | 24.269 | 19.044 | 1.00 | 164.64 |
| 854 | CG | TRP | C | 110 | 16.806 | 25.128 | 18.221 | 1.00 | 164.64 |
| 855 | CD2 | TRP | C | 110 | 16.829 | 26.561 | 18.110 | 1.00 | 164.64 |
| 856 | CE2 | TRP | C | 110 | 15.742 | 26.928 | 17.286 | 1.00 | 164.64 |
| 857 | CE3 | TRP | C | 110 | 17.659 | 27.566 | 18.625 | 1.00 | 164.64 |
| 858 | CD1 | TRP | C | 110 | 15.753 | 24.710 | 17.479 | 1.00 | 164.64 |
| 859 | NE1 | TRP | C | 110 | 15.103 | 25.780 | 16.917 | 1.00 | 164.64 |
| 860 | CZ2 | TRP | C | 110 | 15.460 | 28.261 | 16.964 | 1.00 | 164.64 |
| 861 | CZ3 | TRP | C | 110 | 17.380 | 28.901 | 18.301 | 1.00 | 164.64 |
| 862 | CH2 | TRP | C | 110 | 16.285 | 29.231 | 17.474 | 1.00 | 164.64 |
| 863 | C | TRP | C | 110 | 15.711 | 23.243 | 20.194 | 1.00 | 133.19 |
| 864 | O | TRP | C | 110 | 15.535 | 22.018 | 20.064 | 1.00 | 133.19 |
| 865 | N | ARG | C | 111 | 14.709 | 24.124 | 20.194 | 1.00 | 142.65 |
| 866 | CA | ARG | C | 111 | 13.309 | 23.711 | 20.051 | 1.00 | 142.65 |
| 867 | CB | ARG | C | 111 | 13.020 | 23.259 | 18.618 | 1.00 | 249.69 |
| 868 | CG | ARG | C | 111 | 12.569 | 24.383 | 17.699 | 1.00 | 249.69 |
| 869 | CD | ARG | C | 111 | 11.976 | 23.832 | 16.409 | 1.00 | 249.69 |
| 870 | NE | ARG | C | 111 | 10.799 | 24.593 | 16.004 | 1.00 | 249.69 |
| 871 | CZ | ARG | C | 111 | 9.704 | 24.725 | 16.755 | 1.00 | 249.69 |
| 872 | NH1 | ARG | C | 111 | 9.634 | 24.144 | 17.952 | 1.00 | 249.69 |
| 873 | NH2 | ARG | C | 111 | 8.674 | 25.440 | 16.311 | 1.00 | 249.69 |
| 874 | C | ARG | C | 111 | 12.979 | 22.588 | 21.013 | 1.00 | 142.65 |
| 875 | O | ARG | C | 111 | 12.125 | 21.759 | 20.747 | 1.00 | 142.65 |
| 876 | N | ASN | C | 112 | 13.675 | 22.582 | 22.137 | 1.00 | 230.43 |
| 877 | CA | ASN | C | 112 | 13.477 | 21.574 | 23.156 | 1.00 | 230.43 |
| 878 | CB | ASN | C | 112 | 12.133 | 21.797 | 23.858 | 1.00 | 249.69 |
| 879 | CG | ASN | C | 112 | 12.030 | 21.045 | 25.178 | 1.00 | 249.69 |
| 880 | OD1 | ASN | C | 112 | 12.829 | 20.147 | 25.466 | 1.00 | 249.69 |
| 881 | ND2 | ASN | C | 112 | 11.036 | 21.404 | 25.983 | 1.00 | 249.69 |
| 882 | C | ASN | C | 112 | 13.531 | 20.163 | 22.573 | 1.00 | 230.43 |
| 883 | O | ASN | C | 112 | 12.862 | 19.257 | 23.075 | 1.00 | 230.43 |
| 884 | N | TRP | C | 113 | 14.310 | 19.970 | 21.511 | 1.00 | 206.47 |
| 885 | CA | TRP | C | 113 | 14.426 | 18.637 | 20.916 | 1.00 | 206.47 |
| 886 | CB | TRP | C | 113 | 15.220 | 18.665 | 19.611 | 1.00 | 233.56 |
| 887 | CG | TRP | C | 113 | 14.430 | 19.060 | 18.428 | 1.00 | 233.56 |
| 888 | CD2 | TRP | C | 113 | 14.905 | 19.800 | 17.309 | 1.00 | 233.56 |
| 889 | CE2 | TRP | C | 113 | 13.832 | 19.895 | 16.392 | 1.00 | 233.56 |
| 890 | CE3 | TRP | C | 113 | 16.129 | 20.395 | 16.990 | 1.00 | 233.56 |
| 891 | CD1 | TRP | C | 113 | 13.132 | 18.740 | 18.158 | 1.00 | 233.56 |
| 892 | NE1 | TRP | C | 113 | 12.764 | 19.239 | 16.937 | 1.00 | 233.56 |
| 893 | CZ2 | TRP | C | 113 | 13.952 | 20.566 | 15.166 | 1.00 | 233.56 |
| 894 | CZ3 | TRP | C | 113 | 16.253 | 21.061 | 15.772 | 1.00 | 233.56 |
| 895 | CH2 | TRP | C | 113 | 15.165 | 21.139 | 14.873 | 1.00 | 233.56 |
| 896 | C | TRP | C | 113 | 15.137 | 17.714 | 21.887 | 1.00 | 206.47 |
| 897 | O | TRP | C | 113 | 15.328 | 18.062 | 23.050 | 1.00 | 206.47 |
| 898 | N | ASP | C | 114 | 15.535 | 16.540 | 21.408 | 1.00 | 249.46 |
| 899 | CA | ASP | C | 114 | 16.242 | 15.589 | 22.256 | 1.00 | 249.46 |
| 900 | CB | ASP | C | 114 | 15.542 | 14.218 | 22.229 | 1.00 | 249.69 |
| 901 | CG | ASP | C | 114 | 14.352 | 14.137 | 23.189 | 1.00 | 249.69 |
| 902 | OD1 | ASP | C | 114 | 14.546 | 14.363 | 24.407 | 1.00 | 249.69 |
| 903 | OD2 | ASP | C | 114 | 13.227 | 13.838 | 22.729 | 1.00 | 249.69 |
| 904 | C | ASP | C | 114 | 17.704 | 15.447 | 21.834 | 1.00 | 249.46 |
| 905 | O | ASP | C | 114 | 18.008 | 15.291 | 20.643 | 1.00 | 249.46 |
| 906 | N | VAL | C | 115 | 18.602 | 15.512 | 22.818 | 1.00 | 150.51 |
| 907 | CA | VAL | C | 115 | 20.027 | 15.388 | 22.545 | 1.00 | 150.51 |
| 908 | CB | VAL | C | 115 | 20.831 | 16.572 | 23.147 | 1.00 | 133.46 |
| 909 | CG1 | VAL | C | 115 | 22.243 | 16.592 | 22.569 | 1.00 | 133.46 |
| 910 | CG2 | VAL | C | 115 | 20.140 | 17.884 | 22.859 | 1.00 | 133.46 |
| 911 | C | VAL | C | 115 | 20.559 | 14.078 | 23.135 | 1.00 | 150.51 |
| 912 | O | VAL | C | 115 | 20.153 | 13.669 | 24.225 | 1.00 | 150.51 |
| 913 | N | TYR | C | 116 | 21.468 | 13.432 | 22.404 | 1.00 | 198.57 |
| 914 | CA | TYR | C | 116 | 22.066 | 12.170 | 22.826 | 1.00 | 198.57 |
| 915 | CB | TYR | C | 116 | 21.673 | 11.070 | 21.847 | 1.00 | 249.69 |
| 916 | CG | TYR | C | 116 | 20.185 | 10.787 | 21.832 | 1.00 | 249.69 |
| 917 | CD1 | TYR | C | 116 | 19.350 | 11.339 | 20.856 | 1.00 | 249.69 |
| 918 | CE1 | TYR | C | 116 | 17.974 | 11.099 | 20.866 | 1.00 | 249.69 |
| 919 | CD2 | TYR | C | 116 | 19.608 | 9.987 | 22.816 | 1.00 | 249.69 |
| 920 | CE2 | TYR | C | 116 | 18.241 | 9.741 | 22.838 | 1.00 | 249.69 |
| 921 | CZ | TYR | C | 116 | 17.429 | 10.296 | 21.865 | 1.00 | 249.69 |
| 922 | OH | TYR | C | 116 | 16.075 | 10.047 | 21.894 | 1.00 | 249.69 |
| 923 | C | TYR | C | 116 | 23.582 | 12.257 | 22.914 | 1.00 | 198.57 |
| 924 | O | TYR | C | 116 | 24.174 | 13.239 | 22.455 | 1.00 | 198.57 |
| 925 | N | LYS | C | 117 | 24.198 | 11.230 | 23.500 | 1.00 | 159.55 |
| 926 | CA | LYS | C | 117 | 25.660 | 11.186 | 23.661 | 1.00 | 159.55 |
| 927 | CB | LYS | C | 117 | 26.357 | 10.893 | 22.320 | 1.00 | 249.69 |
| 928 | CG | LYS | C | 117 | 26.455 | 9.408 | 21.969 | 1.00 | 249.69 |
| 929 | CD | LYS | C | 117 | 27.543 | 9.165 | 20.910 | 1.00 | 249.69 |
| 930 | CE | LYS | C | 117 | 28.915 | 9.642 | 21.399 | 1.00 | 249.69 |
| 931 | NZ | LYS | C | 117 | 30.027 | 9.430 | 20.424 | 1.00 | 249.69 |
| 932 | C | LYS | C | 117 | 26.242 | 12.486 | 24.245 | 1.00 | 159.55 |
| 933 | O | LYS | C | 117 | 27.200 | 13.081 | 23.698 | 1.00 | 159.55 |
| 934 | N | VAL | C | 118 | 25.675 | 12.904 | 25.371 | 1.00 | 141.55 |
| 935 | CA | VAL | C | 118 | 26.095 | 14.127 | 26.016 | 1.00 | 141.55 |
| 936 | CB | VAL | C | 118 | 24.919 | 14.737 | 26.790 | 1.00 | 150.57 |
| 937 | CG1 | VAL | C | 118 | 25.416 | 15.626 | 27.908 | 1.00 | 150.57 |
| 938 | CG2 | VAL | C | 118 | 24.067 | 15.543 | 25.830 | 1.00 | 150.57 |
| 939 | C | VAL | C | 118 | 27.305 | 14.028 | 26.927 | 1.00 | 141.55 |
| 940 | O | VAL | C | 118 | 27.466 | 13.067 | 27.672 | 1.00 | 141.55 |
| 941 | N | ILE | C | 119 | 28.140 | 15.062 | 26.850 | 1.00 | 119.98 |
| 942 | CA | ILE | C | 119 | 29.358 | 15.192 | 27.640 | 1.00 | 119.98 |
| 943 | CB | ILE | C | 119 | 30.578 | 14.818 | 26.826 | 1.00 | 122.95 |
| 944 | CG2 | ILE | C | 119 | 31.814 | 14.861 | 27.700 | 1.00 | 122.95 |
| 945 | CG1 | ILE | C | 119 | 30.368 | 13.438 | 26.214 | 1.00 | 122.95 |
| 946 | CD1 | ILE | C | 119 | 31.187 | 13.228 | 24.988 | 1.00 | 122.95 |
| 947 | C | ILE | C | 119 | 29.528 | 16.649 | 28.029 | 1.00 | 119.98 |
| 948 | O | ILE | C | 119 | 29.454 | 17.518 | 27.179 | 1.00 | 119.98 |
| 949 | N | TYR | C | 120 | 29.744 | 16.919 | 29.310 | 1.00 | 136.43 |
| 950 | CA | TYR | C | 120 | 29.962 | 18.289 | 29.759 | 1.00 | 136.43 |
| 951 | CB | TYR | C | 120 | 29.334 | 18.542 | 31.119 | 1.00 | 134.07 |
| 952 | CG | TYR | C | 120 | 27.833 | 18.525 | 31.107 | 1.00 | 134.07 |
| 953 | CD1 | TYR | C | 120 | 27.125 | 17.332 | 31.234 | 1.00 | 134.07 |
| 954 | CE1 | TYR | C | 120 | 25.733 | 17.310 | 31.200 | 1.00 | 134.07 |
| 955 | CD2 | TYR | C | 120 | 27.113 | 19.704 | 30.949 | 1.00 | 134.07 |
| 956 | CE2 | TYR | C | 120 | 25.721 | 19.705 | 30.912 | 1.00 | 134.07 |
| 957 | CZ | TYR | C | 120 | 25.037 | 18.507 | 31.037 | 1.00 | 134.07 |
| 958 | OH | TYR | C | 120 | 23.658 | 18.523 | 30.988 | 1.00 | 134.07 |
| 959 | C | TYR | C | 120 | 31.455 | 18.459 | 29.877 | 1.00 | 136.43 |
| 960 | O | TYR | C | 120 | 32.171 | 17.497 | 30.131 | 1.00 | 136.43 |
| 961 | N | TYR | C | 121 | 31.935 | 19.674 | 29.682 | 1.00 | 132.15 |
| 962 | CA | TYR | C | 121 | 33.366 | 19.916 | 29.782 | 1.00 | 132.15 |
| 963 | CB | TYR | C | 121 | 33.991 | 20.177 | 28.405 | 1.00 | 142.37 |
| 964 | CG | TYR | C | 121 | 34.032 | 19.017 | 27.437 | 1.00 | 142.37 |
| 965 | CD1 | TYR | C | 121 | 32.863 | 18.383 | 27.007 | 1.00 | 142.37 |
| 966 | CE1 | TYR | C | 121 | 32.889 | 17.388 | 26.038 | 1.00 | 142.37 |
| 967 | CD2 | TYR | C | 121 | 35.237 | 18.620 | 26.879 | 1.00 | 142.37 |
| 968 | CE2 | TYR | C | 121 | 35.284 | 17.616 | 25.900 | 1.00 | 142.37 |
| 969 | CZ | TYR | C | 121 | 34.098 | 17.005 | 25.481 | 1.00 | 142.37 |
| 970 | OH | TYR | C | 121 | 34.141 | 16.024 | 24.499 | 1.00 | 142.37 |
| 971 | C | TYR | C | 121 | 33.668 | 21.119 | 30.667 | 1.00 | 132.15 |
| 972 | O | TYR | c | 121 | 33.025 | 22.159 | 30.543 | 1.00 | 132.15 |
| 973 | N | LYS | c | 122 | 34.650 | 20.977 | 31.554 | 1.00 | 126.92 |
| 974 | CA | LYS | c | 122 | 35.056 | 22.076 | 32.413 | 1.00 | 126.92 |
| 975 | CB | LYS | C | 122 | 34.793 | 21.754 | 33.878 | 1.00 | 206.28 |
| 976 | CG | LYS | C | 122 | 35.177 | 22.888 | 34.807 | 1.00 | 206.28 |
| 977 | CD | LYS | C | 122 | 35.209 | 22.420 | 36.238 | 1.00 | 206.28 |
| 978 | CE | LYS | C | 122 | 35.768 | 23.480 | 37.149 | 1.00 | 206.28 |
| 979 | NZ | LYS | C | 122 | 35.923 | 22.930 | 38.515 | 1.00 | 206.28 |
| 980 | C | LYS | C | 122 | 36.544 | 22.299 | 32.193 | 1.00 | 126.92 |
| 981 | O | LYS | C | 122 | 37.349 | 21.420 | 32.461 | 1.00 | 126.92 |
| 982 | N | ASP | C | 123 | 36.902 | 23.477 | 31.699 | 1.00 | 135.65 |
| 983 | CA | ASP | C | 123 | 38.294 | 23.827 | 31.435 | 1.00 | 135.65 |
| 984 | CB | ASP | C | 123 | 39.093 | 23.949 | 32.740 | 1.00 | 170.94 |
| 985 | CG | ASP | C | 123 | 38.763 | 25.212 | 33.507 | 1.00 | 170.94 |
| 986 | OD1 | ASP | C | 123 | 38.780 | 26.308 | 32.904 | 1.00 | 170.94 |
| 987 | OD2 | ASP | C | 123 | 38.496 | 25.117 | 34.721 | 1.00 | 170.94 |
| 988 | C | ASP | C | 123 | 38.987 | 22.847 | 30.509 | 1.00 | 135.65 |
| 989 | O | ASP | C | 123 | 40.082 | 22.372 | 30.818 | 1.00 | 135.65 |
| 990 | N | GLY | C | 124 | 38.345 | 22.547 | 29.378 | 1.00 | 178.68 |
| 991 | CA | GLY | C | 124 | 38.917 | 21.638 | 28.395 | 1.00 | 178.68 |
| 992 | C | GLY | C | 124 | 38.936 | 20.152 | 28.718 | 1.00 | 178.68 |
| 993 | O | GLY | C | 124 | 39.295 | 19.336 | 27.879 | 1.00 | 178.68 |
| 994 | N | GLU | C | 125 | 38.539 | 19.797 | 29.929 | 1.00 | 128.18 |
| 995 | CA | GLU | C | 125 | 38.536 | 18.407 | 30.368 | 1.00 | 128.18 |
| 996 | CB | GLU | C | 125 | 38.963 | 18.324 | 31.847 | 1.00 | 249.69 |
| 997 | CG | GLU | C | 125 | 40.422 | 18.660 | 32.115 | 1.00 | 249.69 |
| 998 | CD | GLU | C | 125 | 41.355 | 17.552 | 31.693 | 1.00 | 249.69 |
| 999 | OE1 | GLU | C | 125 | 41.251 | 16.453 | 32.273 | 1.00 | 249.69 |
| 1000 | OE2 | GLU | C | 125 | 42.189 | 17.777 | 30.786 | 1.00 | 249.69 |
| 1001 | C | GLU | C | 125 | 37.176 | 17.749 | 30.215 | 1.00 | 128.18 |
| 1002 | O | GLU | C | 125 | 36.149 | 18.395 | 30.403 | 1.00 | 128.18 |
| 1003 | N | ALA | C | 126 | 37.165 | 16.468 | 29.866 | 1.00 | 114.67 |
| 1004 | CA | ALA | C | 126 | 35.904 | 15.742 | 29.753 | 1.00 | 114.67 |
| 1005 | CB | ALA | C | 126 | 36.156 | 14.376 | 29.162 | 1.00 | 125.78 |
| 1006 | C | ALA | C | 126 | 35.397 | 15.622 | 31.195 | 1.00 | 114.67 |
| 1007 | O | ALA | C | 126 | 36.190 | 15.374 | 32.099 | 1.00 | 114.67 |
| 1008 | N | LEU | C | 127 | 34.101 | 15.788 | 31.437 | 1.00 | 136.95 |
| 1009 | CA | LEU | C | 127 | 33.633 | 15.718 | 32.816 | 1.00 | 136.95 |
| 1010 | CB | LEU | C | 127 | 33.090 | 17.062 | 33.235 | 1.00 | 112.43 |
| 1011 | CG | LEU | C | 127 | 33.259 | 17.216 | 34.734 | 1.00 | 112.43 |
| 1012 | CD1 | LEU | C | 127 | 34.725 | 17.020 | 35.093 | 1.00 | 112.43 |
| 1013 | CD2 | LEU | C | 127 | 32.768 | 18.588 | 35.170 | 1.00 | 112.43 |
| 1014 | C | LEU | C | 127 | 32.612 | 14.648 | 33.156 | 1.00 | 136.95 |
| 1015 | O | LEU | C | 127 | 32.870 | 13.803 | 34.019 | 1.00 | 136.95 |
| 1016 | N | LYS | C | 128 | 31.444 | 14.696 | 32.518 | 1.00 | 111.10 |
| 1017 | CA | LYS | C | 128 | 30.397 | 13.689 | 32.750 | 1.00 | 111.10 |
| 1018 | CB | LYS | C | 128 | 29.228 | 14.302 | 33.525 | 1.00 | 196.03 |
| 1019 | CG | LYS | C | 128 | 29.586 | 14.833 | 34.905 | 1.00 | 196.03 |
| 1020 | CD | LYS | C | 128 | 29.864 | 13.714 | 35.892 | 1.00 | 196.03 |
| 1021 | CE | LYS | C | 128 | 30.150 | 14.277 | 37.285 | 1.00 | 196.03 |
| 1022 | NZ | LYS | C | 128 | 30.192 | 13.214 | 38.329 | 1.00 | 196.03 |
| 1023 | C | LYS | C | 128 | 29.929 | 13.215 | 31.379 | 1.00 | 111.10 |
| 1024 | O | LYS | C | 128 | 30.196 | 13.874 | 30.360 | 1.00 | 111.10 |
| 1025 | N | TYR | C | 129 | 29.243 | 12.078 | 31.339 | 1.00 | 147.54 |
| 1026 | CA | TYR | C | 129 | 28.753 | 11.567 | 30.058 | 1.00 | 147.54 |
| 1027 | CB | TYR | C | 129 | 29.834 | 10.755 | 29.363 | 1.00 | 149.35 |
| 1028 | CG | TYR | C | 129 | 29.282 | 9.856 | 28.292 | 1.00 | 149.35 |
| 1029 | CD1 | TYR | C | 129 | 29.066 | 10.331 | 27.005 | 1.00 | 149.35 |
| 1030 | CE1 | TYR | C | 129 | 28.507 | 9.509 | 26.029 | 1.00 | 149.35 |
| 1031 | CD2 | TYR | C | 129 | 28.929 | 8.534 | 28.584 | 1.00 | 149.35 |
| 1032 | CE2 | TYR | C | 129 | 28.371 | 7.707 | 27.619 | 1.00 | 149.35 |
| 1033 | CZ | TYR | C | 129 | 28.161 | 8.194 | 26.340 | 1.00 | 149.35 |
| 1034 | OH | TYR | C | 129 | 27.613 | 7.363 | 25.373 | 1.00 | 149.35 |
| 1035 | C | TYR | C | 129 | 27.500 | 10.704 | 30.177 | 1.00 | 147.54 |
| 1036 | O | TYR | C | 129 | 27.384 | 9.893 | 31.098 | 1.00 | 147.54 |
| 1037 | N | TRP | C | 130 | 26.571 | 10.876 | 29.231 | 1.00 | 199.38 |
| 1038 | CA | TRP | C | 130 | 25.323 | 10.109 | 29.216 | 1.00 | 199.38 |
| 1039 | CB | TRP | C | 130 | 24.219 | 10.809 | 30.022 | 1.00 | 218.46 |
| 1040 | CG | TRP | C | 130 | 24.599 | 11.335 | 31.370 | 1.00 | 218.46 |
| 1041 | CD2 | TRP | C | 130 | 24.246 | 10.782 | 32.632 | 1.00 | 218.46 |
| 1042 | CE2 | TRP | C | 130 | 24.770 | 11.627 | 33.631 | 1.00 | 218.46 |
| 1043 | CE3 | TRP | C | 130 | 23.517 | 9.648 | 33.029 | 1.00 | 218.46 |
| 1044 | CD1 | TRP | C | 130 | 25.325 | 12.465 | 31.639 | 1.00 | 218.46 |
| 1045 | NE1 | TRP | C | 130 | 25.431 | 12.652 | 32.992 | 1.00 | 218.46 |
| 1046 | CZ2 | TRP | C | 130 | 24.605 | 11.381 | 34.996 | 1.00 | 218.46 |
| 1047 | CZ3 | TRP | C | 130 | 23.349 | 9.398 | 34.390 | 1.00 | 218.46 |
| 1048 | CH2 | TRP | C | 130 | 23.895 | 10.258 | 35.357 | 1.00 | 218.46 |
| 1049 | C | TRP | C | 130 | 24.794 | 9.910 | 27.794 | 1.00 | 199.38 |
| 1050 | O | TRP | C | 130 | 25.272 | 10.526 | 26.839 | 1.00 | 199.38 |
| 1051 | N | TYR | C | 131 | 23.787 | 9.053 | 27.671 | 1.00 | 229.56 |
| 1052 | CA | TYR | C | 131 | 23.148 | 8.778 | 26.386 | 1.00 | 229.56 |
| 1053 | CB | TYR | C | 131 | 22.591 | 7.356 | 26.372 | 1.00 | 246.37 |
| 1054 | CG | TYR | C | 131 | 22.180 | 6.894 | 24.998 | 1.00 | 246.37 |
| 1055 | CD1 | TYR | C | 131 | 23.142 | 6.593 | 24.031 | 1.00 | 246.37 |
| 1056 | CE1 | TYR | C | 131 | 22.771 | 6.206 | 22.748 | 1.00 | 246.37 |
| 1057 | CD2 | TYR | C | 131 | 20.834 | 6.795 | 24.647 | 1.00 | 246.37 |
| 1058 | CE2 | TYR | C | 131 | 20.454 | 6.411 | 23.370 | 1.00 | 246.37 |
| 1059 | CZ | TYR | C | 131 | 21.422 | 6.120 | 22.425 | 1.00 | 246.37 |
| 1060 | OH | TYR | C | 131 | 21.037 | 5.759 | 21.152 | 1.00 | 246.37 |
| 1061 | C | TYR | C | 131 | 22.003 | 9.783 | 26.290 | 1.00 | 229.56 |
| 1062 | O | TYR | C | 131 | 22.194 | 10.887 | 25.767 | 1.00 | 229.56 |
| 1063 | N | GLU | C | 132 | 20.814 | 9.387 | 26.766 | 1.00 | 249.69 |
| 1064 | CA | GLU | C | 132 | 19.674 | 10.303 | 26.812 | 1.00 | 249.69 |
| 1065 | CB | GLU | C | 132 | 18.455 | 9.632 | 27.444 | 1.00 | 249.69 |
| 1066 | CG | GLU | C | 132 | 17.670 | 8.696 | 26.529 | 1.00 | 249.69 |
| 1067 | CD | GLU | C | 132 | 16.251 | 9.186 | 26.293 | 1.00 | 249.69 |
| 1068 | OE1 | GLU | C | 132 | 15.815 | 10.108 | 27.021 | 1.00 | 249.69 |
| 1069 | OE2 | GLU | C | 132 | 15.566 | 8.651 | 25.388 | 1.00 | 249.69 |
| 1070 | C | GLU | C | 132 | 20.299 | 11.278 | 27.798 | 1.00 | 249.69 |
| 1071 | O | GLU | C | 132 | 20.759 | 10.849 | 28.865 | 1.00 | 249.69 |
| 1072 | N | ASN | C | 133 | 20.318 | 12.573 | 27.483 | 1.00 | 172.36 |
| 1073 | CA | ASN | C | 133 | 21.034 | 13.476 | 28.374 | 1.00 | 172.36 |
| 1074 | CB | ASN | C | 133 | 21.319 | 14.844 | 27.675 | 1.00 | 175.23 |
| 1075 | CG | ASN | C | 133 | 20.166 | 15.840 | 27.735 | 1.00 | 175.23 |
| 1076 | OD1 | ASN | C | 133 | 18.999 | 15.492 | 27.529 | 1.00 | 175.23 |
| 1077 | ND2 | ASN | C | 133 | 20.506 | 17.113 | 27.974 | 1.00 | 175.23 |
| 1078 | C | ASN | C | 133 | 20.565 | 13.626 | 29.815 | 1.00 | 172.36 |
| 1079 | O | ASN | C | 133 | 19.680 | 12.906 | 30.290 | 1.00 | 172.36 |
| 1080 | N | HIS | C | 134 | 21.238 | 14.521 | 30.522 | 1.00 | 165.75 |
| 1081 | CA | HIS | C | 134 | 20.960 | 14.757 | 31.918 | 1.00 | 165.75 |
| 1082 | CB | HIS | C | 134 | 21.912 | 13.910 | 32.756 | 1.00 | 249.69 |
| 1083 | CG | HIS | C | 134 | 21.588 | 13.916 | 34.223 | 1.00 | 249.69 |
| 1084 | CD2 | HIS | C | 134 | 22.305 | 14.372 | 35.277 | 1.00 | 249.69 |
| 1085 | ND1 | HIS | C | 134 | 20.407 | 13.441 | 34.715 | 1.00 | 249.69 |
| 1086 | CE1 | HIS | C | 134 | 20.390 | 13.599 | 36.041 | 1.00 | 249.69 |
| 1087 | NE2 | HIS | C | 134 | 21.526 | 14.161 | 36.397 | 1.00 | 249.69 |
| 1088 | C | HIS | C | 134 | 21.166 | 16.233 | 32.200 | 1.00 | 165.75 |
| 1089 | O | HIS | C | 134 | 21.223 | 17.050 | 31.285 | 1.00 | 165.75 |
| 1090 | N | ASN | C | 135 | 21.278 | 16.572 | 33.470 | 1.00 | 159.28 |
| 1091 | CA | ASN | C | 135 | 21.467 | 17.948 | 33.879 | 1.00 | 159.28 |
| 1092 | CB | ASN | C | 135 | 20.111 | 18.596 | 34.178 | 1.00 | 249.51 |
| 1093 | CG | ASN | C | 135 | 19.266 | 18.779 | 32.923 | 1.00 | 249.51 |
| 1094 | OD1 | ASN | C | 135 | 19.783 | 19.248 | 31.903 | 1.00 | 249.51 |
| 1095 | ND2 | ASN | C | 135 | 17.974 | 18.439 | 32.990 | 1.00 | 249.51 |
| 1096 | C | ASN | C | 135 | 22.374 | 18.001 | 35.116 | 1.00 | 159.28 |
| 1097 | O | ASN | C | 135 | 21.887 | 18.086 | 36.253 | 1.00 | 159.28 |
| 1098 | N | ILE | C | 136 | 23.692 | 17.946 | 34.884 | 1.00 | 134.89 |
| 1099 | CA | ILE | C | 136 | 24.734 | 17.989 | 35.934 | 1.00 | 134.89 |
| 1100 | CB | ILE | C | 136 | 26.090 | 18.399 | 35.318 | 1.00 | 169.29 |
| 1101 | CG2 | ILE | C | 136 | 25.962 | 19.723 | 34.593 | 1.00 | 169.29 |
| 1102 | CG1 | ILE | C | 136 | 27.139 | 18.510 | 36.407 | 1.00 | 169.29 |
| 1103 | CD1 | ILE | C | 136 | 28.472 | 18.980 | 35.884 | 1.00 | 169.29 |
| 1104 | C | ILE | C | 136 | 24.408 | 18.906 | 37.123 | 1.00 | 134.89 |
| 1105 | O | ILE | C | 136 | 24.498 | 20.125 | 37.049 | 1.00 | 134.89 |
| 1106 | N | SER | C | 137 | 24.055 | 18.280 | 38.232 | 1.00 | 137.39 |
| 1107 | CA | SER | C | 137 | 23.678 | 19.000 | 39.422 | 1.00 | 137.39 |
| 1108 | CB | SER | C | 137 | 22.367 | 18.437 | 39.940 | 1.00 | 121.23 |
| 1109 | OG | SER | C | 137 | 22.076 | 18.966 | 41.222 | 1.00 | 121.23 |
| 1110 | C | SER | C | 137 | 24.687 | 19.025 | 40.566 | 1.00 | 137.39 |
| 1111 | O | SER | C | 137 | 25.458 | 18.081 | 40.775 | 1.00 | 137.39 |
| 1112 | N | ILE | C | 138 | 24.647 | 20.113 | 41.331 | 1.00 | 121.18 |
| 1113 | CA | ILE | C | 138 | 25.539 | 20.309 | 42.478 | 1.00 | 121.18 |
| 1114 | CB | ILE | C | 138 | 26.683 | 21.238 | 42.109 | 1.00 | 97.12 |
| 1115 | CG2 | ILE | C | 138 | 27.385 | 21.749 | 43.358 | 1.00 | 97.12 |
| 1116 | CG1 | ILE | C | 138 | 27.646 | 20.502 | 41.187 | 1.00 | 97.12 |
| 1117 | CD1 | ILE | C | 138 | 28.637 | 21.411 | 40.528 | 1.00 | 97.12 |
| 1118 | C | ILE | C | 138 | 24.814 | 20.886 | 43.697 | 1.00 | 121.18 |
| 1119 | O | ILE | C | 138 | 24.212 | 21.966 | 43.651 | 1.00 | 121.18 |
| 1120 | N | THR | C | 139 | 24.890 | 20.145 | 44.796 | 1.00 | 175.86 |
| 1121 | CA | THR | C | 139 | 24.253 | 20.532 | 46.042 | 1.00 | 175.86 |
| 1122 | CB | THR | C | 139 | 24.065 | 19.297 | 46.929 | 1.00 | 224.21 |
| 1123 | OG1 | THR | C | 139 | 25.324 | 18.622 | 47.063 | 1.00 | 224.21 |
| 1124 | CG2 | THR | C | 139 | 23.061 | 18.341 | 46.298 | 1.00 | 224.21 |
| 1125 | C | THR | C | 139 | 25.144 | 21.539 | 46.745 | 1.00 | 175.86 |
| 1126 | O | THR | C | 139 | 24.927 | 22.746 | 46.654 | 1.00 | 175.86 |
| 1127 | N | ASN | C | 140 | 26.149 | 21.024 | 47.447 | 1.00 | 196.19 |
| 1128 | CA | ASN | C | 140 | 27.111 | 21.852 | 48.165 | 1.00 | 196.19 |
| 1129 | CB | ASN | C | 140 | 27.710 | 21.053 | 49.330 | 1.00 | 249.69 |
| 1130 | CG | ASN | C | 140 | 28.741 | 21.837 | 50.109 | 1.00 | 249.69 |
| 1131 | OD1 | ASN | C | 140 | 29.656 | 22.417 | 49.523 | 1.00 | 249.69 |
| 1132 | ND2 | ASN | C | 140 | 28.610 | 21.845 | 51.432 | 1.00 | 249.69 |
| 1133 | C | ASN | C | 140 | 28.184 | 22.213 | 47.146 | 1.00 | 196.19 |
| 1134 | O | ASN | C | 140 | 28.799 | 21.327 | 46.560 | 1.00 | 196.19 |
| 1135 | N | ALA | C | 141 | 28.400 | 23.507 | 46.932 | 1.00 | 134.37 |
| 1136 | CA | ALA | C | 141 | 29.383 | 23.954 | 45.949 | 1.00 | 134.37 |
| 1137 | CB | ALA | C | 141 | 28.834 | 25.131 | 45.148 | 1.00 | 79.03 |
| 1138 | C | ALA | C | 141 | 30.763 | 24.317 | 46.507 | 1.00 | 134.37 |
| 1139 | O | ALA | C | 141 | 30.896 | 25.112 | 47.452 | 1.00 | 134.37 |
| 1140 | N | THR | C | 142 | 31.793 | 23.724 | 45.898 | 1.00 | 119.18 |
| 1141 | CA | THR | C | 142 | 33.183 | 23.954 | 46.281 | 1.00 | 119.18 |
| 1142 | CB | THR | C | 142 | 34.057 | 22.720 | 46.002 | 1.00 | 209.05 |
| 1143 | OG1 | THR | C | 142 | 33.458 | 21.553 | 46.578 | 1.00 | 209.05 |
| 1144 | CG2 | THR | C | 142 | 35.431 | 22.907 | 46.602 | 1.00 | 209.05 |
| 1145 | C | THR | C | 142 | 33.691 | 25.093 | 45.416 | 1.00 | 119.18 |
| 1146 | O | THR | C | 142 | 33.128 | 25.381 | 44.356 | 1.00 | 119.18 |
| 1147 | N | VAL | C | 143 | 34.752 | 25.741 | 45.865 | 1.00 | 145.21 |
| 1148 | CA | VAL | C | 143 | 35.299 | 26.836 | 45.096 | 1.00 | 145.21 |
| 1149 | CB | VAL | C | 143 | 36.295 | 27.672 | 45.910 | 1.00 | 137.97 |
| 1150 | CG1 | VAL | C | 143 | 37.596 | 26.915 | 46.096 | 1.00 | 137.97 |
| 1151 | CG2 | VAL | C | 143 | 36.549 | 28.993 | 45.202 | 1.00 | 137.97 |
| 1152 | C | VAL | C | 143 | 36.023 | 26.287 | 43.879 | 1.00 | 145.21 |
| 1153 | O | VAL | C | 143 | 36.183 | 26.981 | 42.881 | 1.00 | 145.21 |
| 1154 | N | GLU | C | 144 | 36.469 | 25.040 | 43.956 | 1.00 | 198.77 |
| 1155 | CA | GLU | C | 144 | 37.176 | 24.445 | 42.833 | 1.00 | 198.77 |
| 1156 | CB | GLU | C | 144 | 37.858 | 23.142 | 43.241 | 1.00 | 249.69 |
| 1157 | CG | GLU | C | 144 | 38.885 | 23.312 | 44.330 | 1.00 | 249.69 |
| 1158 | CD | GLU | C | 144 | 38.447 | 22.661 | 45.624 | 1.00 | 249.69 |
| 1159 | OE1 | GLU | C | 144 | 38.251 | 21.428 | 45.621 | 1.00 | 249.69 |
| 1160 | OE2 | GLU | C | 144 | 38.293 | 23.377 | 46.639 | 1.00 | 249.69 |
| 1161 | C | GLU | C | 144 | 36.217 | 24.179 | 41.694 | 1.00 | 198.77 |
| 1162 | O | GLU | C | 144 | 36.656 | 23.892 | 40.581 | 1.00 | 198.77 |
| 1163 | N | ASP | C | 145 | 34.912 | 24.274 | 41.969 | 1.00 | 130.51 |
| 1164 | CA | ASP | C | 145 | 33.904 | 24.049 | 40.930 | 1.00 | 130.51 |
| 1165 | CB | ASP | C | 145 | 32.523 | 23.868 | 41.555 | 1.00 | 171.03 |
| 1166 | CG | ASP | C | 145 | 32.326 | 22.490 | 42.136 | 1.00 | 171.03 |
| 1167 | OD1 | ASP | C | 145 | 32.478 | 21.503 | 41.380 | 1.00 | 171.03 |
| 1168 | OD2 | ASP | C | 145 | 32.011 | 22.392 | 43.340 | 1.00 | 171.03 |
| 1169 | C | ASP | C | 145 | 33.863 | 25.213 | 39.939 | 1.00 | 130.51 |
| 1170 | O | ASP | C | 145 | 33.299 | 25.086 | 38.851 | 1.00 | 130.51 |
| 1171 | N | SER | C | 146 | 34.461 | 26.342 | 40.327 | 1.00 | 126.39 |
| 1172 | CA | SER | C | 146 | 34.505 | 27.546 | 39.491 | 1.00 | 126.39 |
| 1173 | CB | SER | C | 146 | 35.120 | 28.723 | 40.270 | 1.00 | 126.60 |
| 1174 | OG | SER | C | 146 | 34.403 | 29.041 | 41.449 | 1.00 | 126.60 |
| 1175 | C | SER | C | 146 | 35.343 | 27.301 | 38.244 | 1.00 | 126.39 |
| 1176 | O | SER | C | 146 | 36.478 | 26.850 | 38.351 | 1.00 | 126.39 |
| 1177 | N | GLY | C | 147 | 34.798 | 27.614 | 37.072 | 1.00 | 222.43 |
| 1178 | CA | GLY | C | 147 | 35.550 | 27.410 | 35.846 | 1.00 | 222.43 |
| 1179 | C | GLY | C | 147 | 34.769 | 27.771 | 34.607 | 1.00 | 222.43 |
| 1180 | O | GLY | C | 147 | 33.801 | 28.520 | 34.682 | 1.00 | 222.43 |
| 1181 | N | THR | C | 148 | 35.187 | 27.236 | 33.463 | 1.00 | 128.05 |
| 1182 | CA | THR | C | 148 | 34.508 | 27.506 | 32.186 | 1.00 | 128.05 |
| 1183 | CB | THR | C | 148 | 35.474 | 28.253 | 31.201 | 1.00 | 134.31 |
| 1184 | OG1 | THR | C | 148 | 36.046 | 27.326 | 30.281 | 1.00 | 134.31 |
| 1185 | CG2 | THR | C | 148 | 36.609 | 28.943 | 31.973 | 1.00 | 134.31 |
| 1186 | C | THR | C | 148 | 33.949 | 26.223 | 31.538 | 1.00 | 128.05 |
| 1187 | O | THR | C | 148 | 34.679 | 25.363 | 31.076 | 1.00 | 128.05 |
| 1188 | N | TYR | C | 149 | 32.634 | 26.114 | 31.517 | 1.00 | 106.87 |
| 1189 | CA | TYR | C | 149 | 31.975 | 24.945 | 30.975 | 1.00 | 106.87 |
| 1190 | CB | TYR | C | 149 | 30.819 | 24.510 | 31.905 | 1.00 | 100.05 |
| 1191 | CG | TYR | C | 149 | 31.204 | 24.226 | 33.336 | 1.00 | 100.05 |
| 1192 | CD1 | TYR | C | 149 | 31.489 | 25.254 | 34.210 | 1.00 | 100.05 |
| 1193 | CE1 | TYR | C | 149 | 31.799 | 24.992 | 35.538 | 1.00 | 100.05 |
| 1194 | CD2 | TYR | C | 149 | 31.247 | 22.922 | 33.816 | 1.00 | 100.05 |
| 1195 | CE2 | TYR | C | 149 | 31.557 | 22.643 | 35.125 | 1.00 | 100.05 |
| 1196 | CZ | TYR | C | 149 | 31.829 | 23.676 | 35.993 | 1.00 | 100.05 |
| 1197 | OH | TYR | C | 149 | 32.111 | 23.390 | 37.325 | 1.00 | 100.05 |
| 1198 | C | TYR | C | 149 | 31.404 | 25.100 | 29.567 | 1.00 | 106.87 |
| 1199 | O | TYR | C | 149 | 31.228 | 26.210 | 29.077 | 1.00 | 106.87 |
| 1200 | N | TYR | C | 150 | 31.105 | 23.953 | 28.946 | 1.00 | 107.82 |
| 1201 | CA | TYR | C | 150 | 30.490 | 23.838 | 27.614 | 1.00 | 107.82 |
| 1202 | CB | TYR | C | 150 | 31.451 | 24.307 | 26.499 | 1.00 | 160.56 |
| 1203 | CG | TYR | C | 150 | 32.523 | 23.336 | 26.058 | 1.00 | 160.56 |
| 1204 | CD1 | TYR | C | 150 | 32.201 | 22.195 | 25.317 | 1.00 | 160.56 |
| 1205 | CE1 | TYR | C | 150 | 33.199 | 21.307 | 24.872 | 1.00 | 160.56 |
| 1206 | CD2 | TYR | C | 150 | 33.873 | 23.576 | 26.351 | 1.00 | 160.56 |
| 1207 | CE2 | TYR | C | 150 | 34.880 | 22.698 | 25.912 | 1.00 | 160.56 |
| 1208 | CZ | TYR | C | 150 | 34.533 | 21.564 | 25.169 | 1.00 | 160.56 |
| 1209 | OH | TYR | C | 150 | 35.507 | 20.693 | 24.719 | 1.00 | 160.56 |
| 1210 | C | TYR | C | 150 | 30.111 | 22.370 | 27.462 | 1.00 | 107.82 |
| 1211 | O | TYR | C | 150 | 30.700 | 21.521 | 28.119 | 1.00 | 107.82 |
| 1212 | N | CYS | C | 151 | 29.112 | 22.074 | 26.641 | 1.00 | 88.46 |
| 1213 | CA | CYS | C | 151 | 28.711 | 20.694 | 26.450 | 1.00 | 88.46 |
| 1214 | C | CYS | C | 151 | 28.660 | 20.293 | 24.976 | 1.00 | 88.46 |
| 1215 | O | CYS | C | 151 | 28.585 | 21.155 | 24.103 | 1.00 | 88.46 |
| 1216 | CB | CYS | C | 151 | 27.359 | 20.425 | 27.087 | 1.00 | 149.34 |
| 1217 | SG | CYS | C | 151 | 25.995 | 21.380 | 26.366 | 1.00 | 149.34 |
| 1218 | N | THR | C | 152 | 28.711 | 18.981 | 24.715 | 1.00 | 145.00 |
| 1219 | CA | THR | C | 152 | 28.675 | 18.446 | 23.358 | 1.00 | 145.00 |
| 1220 | CB | THR | C | 152 | 30.034 | 17.838 | 22.951 | 1.00 | 154.69 |
| 1221 | OG1 | THR | C | 152 | 30.213 | 16.582 | 23.614 | 1.00 | 154.69 |
| 1222 | CG2 | THR | C | 152 | 31.182 | 18.762 | 23.334 | 1.00 | 154.69 |
| 1223 | C | THR | C | 152 | 27.631 | 17.343 | 23.278 | 1.00 | 145.00 |
| 1224 | O | THR | C | 152 | 27.420 | 16.609 | 24.240 | 1.00 | 145.00 |
| 1225 | N | GLY | C | 153 | 26.988 | 17.213 | 22.125 | 1.00 | 161.71 |
| 1226 | CA | GLY | C | 153 | 25.980 | 16.180 | 21.982 | 1.00 | 161.71 |
| 1227 | C | GLY | C | 153 | 25.515 | 15.979 | 20.558 | 1.00 | 161.71 |
| 1228 | O | GLY | C | 153 | 25.830 | 16.783 | 19.670 | 1.00 | 161.71 |
| 1229 | N | LYS | C | 154 | 24.759 | 14.905 | 20.345 | 1.00 | 155.18 |
| 1230 | CA | LYS | C | 154 | 24.249 | 14.582 | 19.022 | 1.00 | 155.18 |
| 1231 | CB | LYS | C | 154 | 24.531 | 13.110 | 18.710 | 1.00 | 249.69 |
| 1232 | CG | LYS | C | 154 | 24.159 | 12.677 | 17.303 | 1.00 | 249.69 |
| 1233 | CD | LYS | C | 154 | 24.540 | 11.227 | 17.071 | 1.00 | 249.69 |
| 1234 | CE | LYS | C | 154 | 24.106 | 10.749 | 15.692 | 1.00 | 249.69 |
| 1235 | NZ | LYS | C | 154 | 24.460 | 9.317 | 15.459 | 1.00 | 249.69 |
| 1236 | C | LYS | C | 154 | 22.748 | 14.876 | 18.895 | 1.00 | 155.18 |
| 1237 | O | LYS | C | 154 | 21.927 | 14.291 | 19.599 | 1.00 | 155.18 |
| 1238 | N | VAL | C | 155 | 22.410 | 15.798 | 17.993 | 1.00 | 207.77 |
| 1239 | CA | VAL | C | 155 | 21.031 | 16.198 | 17.727 | 1.00 | 207.77 |
| 1240 | CB | VAL | C | 155 | 20.918 | 17.729 | 17.614 | 1.00 | 240.62 |
| 1241 | CG1 | VAL | C | 155 | 19.500 | 18.129 | 17.274 | 1.00 | 240.62 |
| 1242 | CG2 | VAL | C | 155 | 21.336 | 18.369 | 18.918 | 1.00 | 240.62 |
| 1243 | C | VAL | C | 155 | 20.663 | 15.572 | 16.392 | 1.00 | 207.77 |
| 1244 | O | VAL | C | 155 | 21.387 | 15.758 | 15.410 | 1.00 | 207.77 |
| 1245 | N | TRP | C | 156 | 19.536 | 14.860 | 16.343 | 1.00 | 218.56 |
| 1246 | CA | TRP | C | 156 | 19.108 | 14.172 | 15.113 | 1.00 | 218.56 |
| 1247 | CB | TRP | C | 156 | 19.013 | 15.124 | 13.897 | 1.00 | 249.69 |
| 1248 | CG | TRP | C | 156 | 17.915 | 16.165 | 13.921 | 1.00 | 249.69 |
| 1249 | CD2 | TRP | C | 156 | 16.500 | 15.936 | 13.835 | 1.00 | 249.69 |
| 1250 | CE2 | TRP | C | 156 | 15.869 | 17.203 | 13.878 | 1.00 | 249.69 |
| 1251 | CE3 | TRP | C | 156 | 15.705 | 14.788 | 13.725 | 1.00 | 249.69 |
| 1252 | CD1 | TRP | C | 156 | 18.077 | 17.523 | 14.010 | 1.00 | 249.69 |
| 1253 | NE1 | TRP | C | 156 | 16.854 | 18.152 | 13.983 | 1.00 | 249.69 |
| 1254 | CZ2 | TRP | C | 156 | 14.481 | 17.354 | 13.816 | 1.00 | 249.69 |
| 1255 | CZ3 | TRP | C | 156 | 14.324 | 14.939 | 13.660 | 1.00 | 249.69 |
| 1256 | CH2 | TRP | C | 156 | 13.728 | 16.214 | 13.709 | 1.00 | 249.69 |
| 1257 | C | TRP | C | 156 | 20.213 | 13.163 | 14.835 | 1.00 | 218.56 |
| 1258 | O | TRP | C | 156 | 20.243 | 12.080 | 15.416 | 1.00 | 218.56 |
| 1259 | N | GLN | C | 157 | 21.130 | 13.548 | 13.949 | 1.00 | 165.87 |
| 1260 | CA | GLN | C | 157 | 22.264 | 12.707 | 13.578 | 1.00 | 165.87 |
| 1261 | CB | GLN | C | 157 | 21.918 | 11.902 | 12.321 | 1.00 | 216.87 |
| 1262 | CG | GLN | C | 157 | 20.967 | 10.737 | 12.583 | 1.00 | 216.87 |
| 1263 | CD | GLN | C | 157 | 21.564 | 9.669 | 13.496 | 1.00 | 216.87 |
| 1264 | OE1 | GLN | C | 157 | 22.561 | 9.032 | 13.154 | 1.00 | 216.87 |
| 1265 | NE2 | GLN | C | 157 | 20.951 | 9.468 | 14.661 | 1.00 | 216.87 |
| 1266 | C | GLN | C | 157 | 23.592 | 13.461 | 13.378 | 1.00 | 165.87 |
| 1267 | O | GLN | C | 157 | 24.495 | 12.979 | 12.705 | 1.00 | 165.87 |
| 1268 | N | LEU | C | 158 | 23.706 | 14.648 | 13.960 | 1.00 | 219.11 |
| 1269 | CA | LEU | C | 158 | 24.940 | 15.419 | 13.858 | 1.00 | 219.11 |
| 1270 | CB | LEU | C | 158 | 24.767 | 16.628 | 12.929 | 1.00 | 248.45 |
| 1271 | CG | LEU | C | 158 | 24.766 | 16.392 | 11.415 | 1.00 | 248.45 |
| 1272 | CD1 | LEU | C | 158 | 25.460 | 17.580 | 10.748 | 1.00 | 248.45 |
| 1273 | CD2 | LEU | C | 158 | 25.498 | 15.110 | 11.054 | 1.00 | 248.45 |
| 1274 | C | LEU | C | 158 | 25.415 | 15.892 | 15.230 | 1.00 | 219.11 |
| 1275 | O | LEU | C | 158 | 24.619 | 16.116 | 16.136 | 1.00 | 219.11 |
| 1276 | N | ASP | C | 159 | 26.724 | 16.040 | 15.374 | 1.00 | 202.98 |
| 1277 | CA | ASP | C | 159 | 27.314 | 16.485 | 16.629 | 1.00 | 202.98 |
| 1278 | CB | ASP | C | 159 | 28.746 | 15.957 | 16.757 | 1.00 | 249.69 |
| 1279 | CG | ASP | C | 159 | 28.834 | 14.458 | 16.563 | 1.00 | 249.69 |
| 1280 | OD1 | ASP | C | 159 | 28.215 | 13.718 | 17.356 | 1.00 | 249.69 |
| 1281 | OD2 | ASP | C | 159 | 29.522 | 14.017 | 15.616 | 1.00 | 249.69 |
| 1282 | C | ASP | C | 159 | 27.341 | 18.007 | 16.704 | 1.00 | 202.98 |
| 1283 | O | ASP | C | 159 | 27.474 | 18.690 | 15.682 | 1.00 | 202.98 |
| 1284 | N | TYR | C | 160 | 27.209 | 18.541 | 17.915 | 1.00 | 193.11 |
| 1285 | CA | TYR | C | 160 | 27.246 | 19.988 | 18.104 | 1.00 | 193.11 |
| 1286 | CB | TYR | C | 160 | 25.852 | 20.584 | 18.043 | 1.00 | 187.38 |
| 1287 | CG | TYR | C | 160 | 25.114 | 20.253 | 16.778 | 1.00 | 187.38 |
| 1288 | CD1 | TYR | C | 160 | 24.305 | 19.113 | 16.702 | 1.00 | 187.38 |
| 1289 | CE1 | TYR | C | 160 | 23.579 | 18.813 | 15.553 | 1.00 | 187.38 |
| 1290 | CD2 | TYR | C | 160 | 25.195 | 21.087 | 15.665 | 1.00 | 187.38 |
| 1291 | CE2 | TYR | C | 160 | 24.479 | 20.796 | 14.504 | 1.00 | 187.38 |
| 1292 | CZ | TYR | C | 160 | 23.666 | 19.657 | 14.458 | 1.00 | 187.38 |
| 1293 | OH | TYR | C | 160 | 22.920 | 19.374 | 13.337 | 1.00 | 187.38 |
| 1294 | C | TYR | C | 160 | 27.895 | 20.388 | 19.413 | 1.00 | 193.11 |
| 1295 | O | TYR | C | 160 | 27.769 | 19.705 | 20.429 | 1.00 | 193.11 |
| 1296 | N | GLU | C | 161 | 28.585 | 21.519 | 19.360 | 1.00 | 171.02 |
| 1297 | CA | GLU | C | 161 | 29.296 | 22.064 | 20.494 | 1.00 | 171.02 |
| 1298 | CB | GLU | C | 161 | 30.740 | 22.358 | 20.052 | 1.00 | 238.76 |
| 1299 | CG | GLU | C | 161 | 31.660 | 22.975 | 21.085 | 1.00 | 238.76 |
| 1300 | CD | GLU | C | 161 | 33.121 | 22.878 | 20.671 | 1.00 | 238.76 |
| 1301 | OE1 | GLU | C | 161 | 33.948 | 23.655 | 21.196 | 1.00 | 238.76 |
| 1302 | OE2 | GLU | C | 161 | 33.445 | 22.015 | 19.827 | 1.00 | 238.76 |
| 1303 | C | GLU | C | 161 | 28.560 | 23.321 | 20.952 | 1.00 | 171.02 |
| 1304 | O | GLU | C | 161 | 28.044 | 24.068 | 20.135 | 1.00 | 171.02 |
| 1305 | N | SER | C | 162 | 28.500 | 23.533 | 22.263 | 1.00 | 160.21 |
| 1306 | CA | SER | C | 162 | 27.820 | 24.693 | 22.840 | 1.00 | 160.21 |
| 1307 | CB | SER | C | 162 | 27.182 | 24.308 | 24.174 | 1.00 | 143.02 |
| 1308 | OG | SER | C | 162 | 28.169 | 23.903 | 25.109 | 1.00 | 143.02 |
| 1309 | C | SER | C | 162 | 28.767 | 25.856 | 23.078 | 1.00 | 160.21 |
| 1310 | O | SER | C | 162 | 29.978 | 25.678 | 23.147 | 1.00 | 160.21 |
| 1311 | N | GLU | C | 163 | 28.211 | 27.053 | 23.200 | 1.00 | 142.78 |
| 1312 | CA | GLU | C | 163 | 29.043 | 28.207 | 23.471 | 1.00 | 142.78 |
| 1313 | CB | GLU | C | 163 | 28.195 | 29.492 | 23.498 | 1.00 | 247.65 |
| 1314 | CG | GLU | C | 163 | 27.742 | 30.009 | 22.130 | 1.00 | 247.65 |
| 1315 | CD | GLU | C | 163 | 28.870 | 30.651 | 21.331 | 1.00 | 247.65 |
| 1316 | OE1 | GLU | C | 163 | 29.506 | 31.604 | 21.841 | 1.00 | 247.65 |
| 1317 | OE2 | GLU | C | 163 | 29.114 | 30.207 | 20.189 | 1.00 | 247.65 |
| 1318 | C | GLU | C | 163 | 29.632 | 27.931 | 24.857 | 1.00 | 142.78 |
| 1319 | O | GLU | C | 163 | 28.995 | 27.254 | 25.666 | 1.00 | 142.78 |
| 1320 | N | PRO | C | 164 | 30.843 | 28.429 | 25.144 | 1.00 | 104.76 |
| 1321 | CD | PRO | C | 164 | 31.762 | 29.158 | 24.241 | 1.00 | 198.66 |
| 1322 | CA | PRO | C | 164 | 31.481 | 28.209 | 26.442 | 1.00 | 104.76 |
| 1323 | CB | PRO | C | 164 | 32.947 | 28.445 | 26.144 | 1.00 | 198.66 |
| 1324 | CG | PRO | C | 164 | 32.874 | 29.581 | 25.180 | 1.00 | 198.66 |
| 1325 | C | PRO | C | 164 | 30.943 | 29.170 | 27.501 | 1.00 | 104.76 |
| 1326 | O | PRO | C | 164 | 30.623 | 30.318 | 27.189 | 1.00 | 104.76 |
| 1327 | N | LEU | C | 165 | 30.872 | 28.719 | 28.751 | 1.00 | 150.81 |
| 1328 | CA | LEU | C | 165 | 30.352 | 29.562 | 29.820 | 1.00 | 150.81 |
| 1329 | CB | LEU | C | 165 | 28.962 | 29.067 | 30.202 | 1.00 | 114.56 |
| 1330 | CG | LEU | C | 165 | 28.295 | 29.843 | 31.329 | 1.00 | 114.56 |
| 1331 | CD1 | LEU | C | 165 | 28.627 | 31.336 | 31.179 | 1.00 | 114.56 |
| 1332 | CD2 | LEU | C | 165 | 26.778 | 29.586 | 31.296 | 1.00 | 114.56 |
| 1333 | C | LEU | C | 165 | 31.213 | 29.644 | 31.065 | 1.00 | 150.81 |
| 1334 | O | LEU | C | 165 | 31.648 | 28.620 | 31.589 | 1.00 | 150.81 |
| 1335 | N | ASN | C | 166 | 31.445 | 30.864 | 31.538 | 1.00 | 123.03 |
| 1336 | CA | ASN | C | 166 | 32.247 | 31.058 | 32.744 | 1.00 | 123.03 |
| 1337 | CB | ASN | C | 166 | 32.969 | 32.409 | 32.716 | 1.00 | 146.40 |
| 1338 | CG | ASN | C | 166 | 34.388 | 32.314 | 32.177 | 1.00 | 146.40 |
| 1339 | OD1 | ASN | C | 166 | 35.031 | 31.273 | 32.254 | 1.00 | 146.40 |
| 1340 | ND2 | ASN | C | 166 | 34.888 | 33.429 | 31.660 | 1.00 | 146.40 |
| 1341 | C | ASN | C | 166 | 31.402 | 30.985 | 34.019 | 1.00 | 123.03 |
| 1342 | O | ASN | C | 166 | 30.257 | 31.409 | 34.022 | 1.00 | 123.03 |
| 1343 | N | ILE | C | 167 | 31.976 | 30.458 | 35.103 | 1.00 | 149.03 |
| 1344 | CA | ILE | C | 167 | 31.266 | 30.339 | 36.374 | 1.00 | 149.03 |
| 1345 | CB | ILE | C | 167 | 30.670 | 28.946 | 36.551 | 1.00 | 98.22 |
| 1346 | CG2 | ILE | C | 167 | 30.085 | 28.799 | 37.947 | 1.00 | 98.22 |
| 1347 | CG1 | ILE | C | 167 | 29.610 | 28.707 | 35.473 | 1.00 | 98.22 |
| 1348 | CD1 | ILE | C | 167 | 29.025 | 27.336 | 35.526 | 1.00 | 98.22 |
| 1349 | C | ILE | C | 167 | 32.178 | 30.592 | 37.548 | 1.00 | 149.03 |
| 1350 | O | ILE | C | 167 | 33.233 | 29.983 | 37.667 | 1.00 | 149.03 |
| 1351 | N | THR | C | 168 | 31.755 | 31.471 | 38.440 | 1.00 | 107.43 |
| 1352 | CA | THR | C | 168 | 32.586 | 31.792 | 39.591 | 1.00 | 107.43 |
| 1353 | CB | THR | C | 168 | 33.120 | 33.225 | 39.487 | 1.00 | 120.91 |
| 1354 | OG1 | THR | C | 168 | 33.823 | 33.372 | 38.246 | 1.00 | 120.91 |
| 1355 | CG2 | THR | C | 168 | 34.054 | 33.526 | 40.633 | 1.00 | 120.91 |
| 1356 | C | THR | C | 168 | 31.955 | 31.603 | 40.965 | 1.00 | 107.43 |
| 1357 | O | THR | C | 168 | 30.943 | 32.192 | 41.312 | 1.00 | 107.43 |
| 1358 | N | VAL | C | 169 | 32.594 | 30.761 | 41.750 | 1.00 | 107.46 |
| 1359 | CA | VAL | C | 169 | 32.152 | 30.470 | 43.092 | 1.00 | 107.46 |
| 1360 | CB | VAL | C | 169 | 32.206 | 28.935 | 43.374 | 1.00 | 105.06 |
| 1361 | CG1 | VAL | C | 169 | 32.281 | 28.657 | 44.853 | 1.00 | 105.06 |
| 1362 | CG2 | VAL | C | 169 | 30.966 | 28.269 | 42.794 | 1.00 | 105.06 |
| 1363 | C | VAL | C | 169 | 33.083 | 31.217 | 44.025 | 1.00 | 107.46 |
| 1364 | O | VAL | C | 169 | 34.266 | 30.874 | 44.135 | 1.00 | 107.46 |
| 1365 | N | ILE | C | 170 | 32.548 | 32.248 | 44.677 | 1.00 | 143.55 |
| 1366 | CA | ILE | C | 170 | 33.320 | 33.068 | 45.614 | 1.00 | 143.55 |
| 1367 | CB | ILE | C | 170 | 32.910 | 34.549 | 45.488 | 1.00 | 150.86 |
| 1368 | CG2 | ILE | C | 170 | 32.957 | 34.967 | 44.028 | 1.00 | 150.86 |
| 1369 | CG1 | ILE | C | 170 | 31.487 | 34.741 | 46.000 | 1.00 | 150.86 |
| 1370 | CD1 | ILE | C | 170 | 31.020 | 36.188 | 45.961 | 1.00 | 150.86 |
| 1371 | C | ILE | C | 170 | 33.102 | 32.586 | 47.056 | 1.00 | 143.55 |
| 1372 | O | ILE | C | 170 | 32.173 | 31.824 | 47.309 | 1.00 | 143.55 |
| 1373 | N | LYS | C | 171 | 33.939 | 33.028 | 47.994 | 1.00 | 170.19 |
| 1374 | CA | LYS | C | 171 | 33.795 | 32.588 | 49.379 | 1.00 | 170.19 |
| 1375 | CB | LYS | C | 171 | 35.038 | 31.812 | 49.790 | 1.00 | 247.79 |
| 1376 | CG | LYS | C | 171 | 36.307 | 32.611 | 49.598 | 1.00 | 247.79 |
| 1377 | CD | LYS | C | 171 | 37.503 | 31.712 | 49.375 | 1.00 | 247.79 |
| 1378 | CE | LYS | C | 171 | 37.723 | 30.756 | 50.537 | 1.00 | 247.79 |
| 1379 | NZ | LYS | C | 171 | 38.942 | 29.921 | 50.335 | 1.00 | 247.79 |
| 1380 | C | LYS | C | 171 | 33.539 | 33.715 | 50.378 | 1.00 | 170.19 |
| 1381 | O | LYS | C | 171 | 33.540 | 33.498 | 51.596 | 1.00 | 170.19 |
| 1382 | C1 | NAG | C | 221 | 5.113 | 30.265 | 25.361 | 1.00 | 249.69 |
| 1383 | C2 | NAG | C | 221 | 5.275 | 28.765 | 25.132 | 1.00 | 249.69 |
| 1384 | N2 | NAG | C | 221 | 6.660 | 28.481 | 24.798 | 1.00 | 249.69 |
| 1385 | C7 | NAG | C | 221 | 7.164 | 27.267 | 25.015 | 1.00 | 249.69 |
| 1386 | O7 | NAG | C | 221 | 6.500 | 26.331 | 25.485 | 1.00 | 249.69 |
| 1387 | C8 | NAG | C | 221 | 8.624 | 27.050 | 24.648 | 1.00 | 249.69 |
| 1388 | C3 | NAG | C | 221 | 4.349 | 28.288 | 24.010 | 1.00 | 249.69 |
| 1389 | O3 | NAG | C | 221 | 4.386 | 26.868 | 23.925 | 1.00 | 249.69 |
| 1390 | C4 | NAG | C | 221 | 2.899 | 28.741 | 24.228 | 1.00 | 249.69 |
| 1391 | O4 | NAG | C | 221 | 2.183 | 28.474 | 23.002 | 1.00 | 249.69 |
| 1392 | C5 | NAG | C | 221 | 2.851 | 30.255 | 24.559 | 1.00 | 249.69 |
| 1393 | O5 | NAG | C | 221 | 3.741 | 30.568 | 25.655 | 1.00 | 249.69 |
| 1394 | C6 | NAG | C | 221 | 1.472 | 30.743 | 24.975 | 1.00 | 249.69 |
| 1395 | O6 | NAG | C | 221 | 0.977 | 30.009 | 26.087 | 1.00 | 249.69 |
| 1396 | C1 | NAG | C | 222 | 0.788 | 28.434 | 23.006 | 1.00 | 249.69 |
| 1397 | C2 | NAG | C | 222 | 0.312 | 27.230 | 22.166 | 1.00 | 249.69 |
| 1398 | N2 | NAG | C | 222 | 0.806 | 25.988 | 22.749 | 1.00 | 249.69 |
| 1399 | C7 | NAG | C | 222 | −0.041 | 25.044 | 23.166 | 1.00 | 249.69 |
| 1400 | O7 | NAG | C | 222 | −1.270 | 25.153 | 23.088 | 1.00 | 249.69 |
| 1401 | C8 | NAG | C | 222 | 0.570 | 23.783 | 23.761 | 1.00 | 249.69 |
| 1402 | C3 | NAG | C | 222 | 0.819 | 27.382 | 20.711 | 1.00 | 249.69 |
| 1403 | O3 | NAG | C | 222 | 0.285 | 26.347 | 19.894 | 1.00 | 249.69 |
| 1404 | C4 | NAG | C | 222 | 0.422 | 28.755 | 20.130 | 1.00 | 249.69 |
| 1405 | O4 | NAG | C | 222 | 1.038 | 28.935 | 18.860 | 1.00 | 249.69 |
| 1406 | C5 | NAG | C | 222 | 0.860 | 29.881 | 21.083 | 1.00 | 249.69 |
| 1407 | O5 | NAG | C | 222 | 0.308 | 29.658 | 22.408 | 1.00 | 249.69 |
| 1408 | C6 | NAG | C | 222 | 0.423 | 31.266 | 20.635 | 1.00 | 249.69 |
| 1409 | O6 | NAG | C | 222 | 1.512 | 32.185 | 20.656 | 1.00 | 249.69 |
| 1410 | C1 | NAG | C | 242 | 18.968 | 46.404 | 25.932 | 1.00 | 249.69 |
| 1411 | C2 | NAG | C | 242 | 18.118 | 46.230 | 24.662 | 1.00 | 249.69 |
| 1412 | N2 | NAG | C | 242 | 16.700 | 46.211 | 24.968 | 1.00 | 249.69 |
| 1413 | C7 | NAG | C | 242 | 15.905 | 47.139 | 24.446 | 1.00 | 249.69 |
| 1414 | O7 | NAG | C | 242 | 16.318 | 48.033 | 23.707 | 1.00 | 249.69 |
| 1415 | C8 | NAG | C | 242 | 14.433 | 47.071 | 24.794 | 1.00 | 249.69 |
| 1416 | C3 | NAG | C | 242 | 18.532 | 44.931 | 23.964 | 1.00 | 249.69 |
| 1417 | O3 | NAG | C | 242 | 17.775 | 44.760 | 22.773 | 1.00 | 249.69 |
| 1418 | C4 | NAG | C | 242 | 20.036 | 44.983 | 23.636 | 1.00 | 249.69 |
| 1419 | O4 | NAG | C | 242 | 20.457 | 43.699 | 23.125 | 1.00 | 249.69 |
| 1420 | C5 | NAG | C | 242 | 20.872 | 45.340 | 24.894 | 1.00 | 249.69 |
| 1421 | O5 | NAG | C | 242 | 20.352 | 46.526 | 25.559 | 1.00 | 249.69 |
| 1422 | C6 | NAG | C | 242 | 22.318 | 45.643 | 24.539 | 1.00 | 249.69 |
| 1423 | O6 | NAG | C | 242 | 23.194 | 44.624 | 24.998 | 1.00 | 249.69 |
| 1424 | C1 | NAG | C | 243 | 21.000 | 43.678 | 21.849 | 1.00 | 249.69 |
| 1425 | C2 | NAG | C | 243 | 21.827 | 42.403 | 21.660 | 1.00 | 249.69 |
| 1426 | N2 | NAG | C | 243 | 22.908 | 42.331 | 22.621 | 1.00 | 249.69 |
| 1427 | C7 | NAG | C | 243 | 23.110 | 41.201 | 23.298 | 1.00 | 249.69 |
| 1428 | O7 | NAG | C | 243 | 22.404 | 40.193 | 23.157 | 1.00 | 249.69 |
| 1429 | C8 | NAG | C | 243 | 24.264 | 41.186 | 24.287 | 1.00 | 249.69 |
| 1430 | C3 | NAG | C | 243 | 22.382 | 42.377 | 20.246 | 1.00 | 249.69 |
| 1431 | O3 | NAG | C | 243 | 23.150 | 41.195 | 20.045 | 1.00 | 249.69 |
| 1432 | C4 | NAG | C | 243 | 21.223 | 42.406 | 19.276 | 1.00 | 249.69 |
| 1433 | O4 | NAG | C | 243 | 21.794 | 42.333 | 17.983 | 1.00 | 249.69 |
| 1434 | C5 | NAG | C | 243 | 20.366 | 43.682 | 19.518 | 1.00 | 249.69 |
| 1435 | O5 | NAG | C | 243 | 19.915 | 43.690 | 20.906 | 1.00 | 249.69 |
| 1436 | C6 | NAG | C | 243 | 19.112 | 43.738 | 18.662 | 1.00 | 249.69 |
| 1437 | O6 | NAG | C | 243 | 18.229 | 42.666 | 18.966 | 1.00 | 249.69 |
| 1438 | C1 | MAN | C | 244 | 21.150 | 41.717 | 16.941 | 1.00 | 247.75 |
| 1439 | C2 | MAN | C | 244 | 21.485 | 42.608 | 15.841 | 1.00 | 247.75 |
| 1440 | O2 | MAN | C | 244 | 22.880 | 42.966 | 15.943 | 1.00 | 247.75 |
| 1441 | C3 | MAN | C | 244 | 21.041 | 42.012 | 14.541 | 1.00 | 247.75 |
| 1442 | O3 | MAN | C | 244 | 21.229 | 42.927 | 13.482 | 1.00 | 247.75 |
| 1443 | C4 | MAN | C | 244 | 21.699 | 40.671 | 14.305 | 1.00 | 247.75 |
| 1444 | O4 | MAN | C | 244 | 21.301 | 40.157 | 13.050 | 1.00 | 247.75 |
| 1445 | C5 | MAN | C | 244 | 21.269 | 39.743 | 15.442 | 1.00 | 247.75 |
| 1446 | 05 | MAN | C | 244 | 21.734 | 40.330 | 16.721 | 1.00 | 247.75 |
| 1447 | C6 | MAN | C | 244 | 21.705 | 38.271 | 15.255 | 1.00 | 247.75 |
| 1448 | 06 | MAN | C | 244 | 23.038 | 38.030 | 15.676 | 1.00 | 247.75 |
| 1449 | C1 | NAG | C | 250 | 0.024 | 39.200 | 37.140 | 1.00 | 249.69 |
| 1450 | C2 | NAG | C | 250 | −0.633 | 37.995 | 37.844 | 1.00 | 249.69 |
| 1451 | N2 | NAG | C | 250 | −0.363 | 38.033 | 39.271 | 1.00 | 249.69 |
| 1452 | C7 | NAG | C | 250 | −1.342 | 38.313 | 40.126 | 1.00 | 249.69 |
| 1453 | O7 | NAG | C | 250 | −2.500 | 38.550 | 39.764 | 1.00 | 249.69 |
| 1454 | C8 | NAG | C | 250 | −0.985 | 38.336 | 41.607 | 1.00 | 249.69 |
| 1455 | C3 | NAG | C | 250 | −0.084 | 36.691 | 37.242 | 1.00 | 249.69 |
| 1456 | O3 | NAG | C | 250 | −0.751 | 35.573 | 37.814 | 1.00 | 249.69 |
| 1457 | C4 | NAG | C | 250 | −0.273 | 36.691 | 35.716 | 1.00 | 249.69 |
| 1458 | O4 | NAG | C | 250 | 0.355 | 35.542 | 35.160 | 1.00 | 249.69 |
| 1459 | C5 | NAG | C | 250 | 0.338 | 37.973 | 35.105 | 1.00 | 249.69 |
| 1460 | O5 | NAG | C | 250 | −0.235 | 39.149 | 35.731 | 1.00 | 249.69 |
| 1461 | C6 | NAG | C | 250 | 0.100 | 38.106 | 33.606 | 1.00 | 249.69 |
| 1462 | O6 | NAG | C | 250 | 0.341 | 39.435 | 33.163 | 1.00 | 249.69 |
| 1463 | C1 | NAG | C | 274 | 17.463 | 53.378 | 50.102 | 1.00 | 249.69 |
| 1464 | C2 | NAG | C | 274 | 18.624 | 52.801 | 50.945 | 1.00 | 249.69 |
| 1465 | N2 | NAG | C | 274 | 18.123 | 51.805 | 51.883 | 1.00 | 249.69 |
| 1466 | C7 | NAG | C | 274 | 18.919 | 50.834 | 52.330 | 1.00 | 249.69 |
| 1467 | O7 | NAG | C | 274 | 20.099 | 50.723 | 51.992 | 1.00 | 249.69 |
| 1468 | C8 | NAG | C | 274 | 18.316 | 49.836 | 53.303 | 1.00 | 249.69 |
| 1469 | C3 | NAG | C | 274 | 19.337 | 53.945 | 51.704 | 1.00 | 249.69 |
| 1470 | O3 | NAG | C | 274 | 20.487 | 53.442 | 52.377 | 1.00 | 249.69 |
| 1471 | C4 | NAG | C | 274 | 19.755 | 55.062 | 50.730 | 1.00 | 249.69 |
| 1472 | O4 | NAG | C | 274 | 20.286 | 56.164 | 51.457 | 1.00 | 249.69 |
| 1473 | C5 | NAG | C | 274 | 18.548 | 55.520 | 49.899 | 1.00 | 249.69 |
| 1474 | O5 | NAG | C | 274 | 17.957 | 54.391 | 49.203 | 1.00 | 249.69 |
| 1475 | C6 | NAG | C | 274 | 18.929 | 56.550 | 48.849 | 1.00 | 249.69 |
| 1476 | O6 | NAG | C | 274 | 17.844 | 56.817 | 47.970 | 1.00 | 249.69 |
| 1477 | C1 | NAG | C | 335 | 16.958 | 19.435 | 32.669 | 1.00 | 249.69 |
| 1478 | C2 | NAG | C | 335 | 15.937 | 19.674 | 33.820 | 1.00 | 249.69 |
| 1479 | N2 | NAG | C | 335 | 16.535 | 19.244 | 35.073 | 1.00 | 249.69 |
| 1480 | C7 | NAG | C | 335 | 16.783 | 20.124 | 36.042 | 1.00 | 249.69 |
| 1481 | O7 | NAG | C | 335 | 16.517 | 21.327 | 35.947 | 1.00 | 249.69 |
| 1482 | C8 | NAG | C | 335 | 17.416 | 19.588 | 37.314 | 1.00 | 249.69 |
| 1483 | C3 | NAG | C | 335 | 14.586 | 18.951 | 33.638 | 1.00 | 249.69 |
| 1484 | O3 | NAG | C | 335 | 13.605 | 19.572 | 34.457 | 1.00 | 249.69 |
| 1485 | C4 | NAG | C | 335 | 14.117 | 18.995 | 32.190 | 1.00 | 249.69 |
| 1486 | O4 | NAG | C | 335 | 12.912 | 18.250 | 32.042 | 1.00 | 249.69 |
| 1487 | C5 | NAG | C | 335 | 15.219 | 18.405 | 31.318 | 1.00 | 249.69 |
| 1488 | O5 | NAG | C | 335 | 16.370 | 19.273 | 31.353 | 1.00 | 249.69 |
| 1489 | C6 | NAG | C | 335 | 14.799 | 18.275 | 29.862 | 1.00 | 249.69 |
| 1490 | O6 | NAG | C | 335 | 14.956 | 16.942 | 29.398 | 1.00 | 249.69 |
| 1491 | C1 | NAG | C | 340 | 29.647 | 21.246 | 52.250 | 1.00 | 249.46 |
| 1492 | C2 | NAG | C | 340 | 30.433 | 22.313 | 53.032 | 1.00 | 249.46 |
| 1493 | N2 | NAG | C | 340 | 30.974 | 23.304 | 52.117 | 1.00 | 249.46 |
| 1494 | C7 | NAG | C | 340 | 30.836 | 24.605 | 52.373 | 1.00 | 249.46 |
| 1495 | O7 | NAG | C | 340 | 30.269 | 25.044 | 53.381 | 1.00 | 249.46 |
| 1496 | C8 | NAG | C | 340 | 31.425 | 25.569 | 51.356 | 1.00 | 249.46 |
| 1497 | C3 | NAG | C | 340 | 31.568 | 21.625 | 53.818 | 1.00 | 249.46 |
| 1498 | O3 | NAG | C | 340 | 32.255 | 22.575 | 54.628 | 1.00 | 249.46 |
| 1499 | C4 | NAG | C | 340 | 30.996 | 20.503 | 54.702 | 1.00 | 249.46 |
| 1500 | O4 | NAG | C | 340 | 32.063 | 19.789 | 55.308 | 1.00 | 249.46 |
| 1501 | C5 | NAG | C | 340 | 30.136 | 19.545 | 53.853 | 1.00 | 249.46 |
| 1502 | O5 | NAG | C | 340 | 29.101 | 20.280 | 53.154 | 1.00 | 249.46 |
| 1503 | C6 | NAG | C | 340 | 29.442 | 18.463 | 54.660 | 1.00 | 249.46 |
| 1504 | O6 | NAG | C | 340 | 28.518 | 17.737 | 53.851 | 1.00 | 249.46 |
| 1505 | C1 | NAG | C | 366 | 36.171 | 33.414 | 30.999 | 1.00 | 209.37 |
| 1506 | C2 | NAG | C | 366 | 36.136 | 34.345 | 29.797 | 1.00 | 209.37 |
| 1507 | N2 | NAG | C | 366 | 35.092 | 33.912 | 28.886 | 1.00 | 209.37 |
| 1508 | C7 | NAG | C | 366 | 33.862 | 34.405 | 28.999 | 1.00 | 209.37 |
| 1509 | O7 | NAG | C | 366 | 33.555 | 35.244 | 29.848 | 1.00 | 209.37 |
| 1510 | C8 | NAG | C | 366 | 32.813 | 33.903 | 28.017 | 1.00 | 209.37 |
| 1511 | C3 | NAG | C | 366 | 37.487 | 34.322 | 29.088 | 1.00 | 209.37 |
| 1512 | O3 | NAG | C | 366 | 37.518 | 35.319 | 28.073 | 1.00 | 209.37 |
| 1513 | C4 | NAG | C | 366 | 38.646 | 34.557 | 30.067 | 1.00 | 209.37 |
| 1514 | O4 | NAG | C | 366 | 39.884 | 34.256 | 29.386 | 1.00 | 209.37 |
| 1515 | C5 | NAG | C | 366 | 38.505 | 33.652 | 31.302 | 1.00 | 209.37 |
| 1516 | O5 | NAG | C | 366 | 37.207 | 33.813 | 31.891 | 1.00 | 209.37 |
| 1517 | C6 | NAG | C | 366 | 39.518 | 33.935 | 32.390 | 1.00 | 209.37 |
| 1518 | O6 | NAG | C | 366 | 39.449 | 32.957 | 33.413 | 1.00 | 209.37 |
| 1519 | C1 | NAG | C | 367 | 40.870 | 35.232 | 29.397 | 1.00 | 249.69 |
| 1520 | C2 | NAG | C | 367 | 42.234 | 34.596 | 29.111 | 1.00 | 249.69 |
| 1521 | N2 | NAG | C | 367 | 42.528 | 33.546 | 30.070 | 1.00 | 249.69 |
| 1522 | C7 | NAG | C | 367 | 42.583 | 32.277 | 29.668 | 1.00 | 249.69 |
| 1523 | O7 | NAG | C | 367 | 42.394 | 31.931 | 28.498 | 1.00 | 249.69 |
| 1524 | C8 | NAG | C | 367 | 42.895 | 31.227 | 30.725 | 1.00 | 249.69 |
| 1525 | C3 | NAG | C | 367 | 43.292 | 35.695 | 29.166 | 1.00 | 249.69 |
| 1526 | O3 | NAG | C | 367 | 44.574 | 35.149 | 28.892 | 1.00 | 249.69 |
| 1527 | C4 | NAG | C | 367 | 42.950 | 36.779 | 28.132 | 1.00 | 249.69 |
| 1528 | O4 | NAG | C | 367 | 43.876 | 37.854 | 28.245 | 1.00 | 249.69 |
| 1529 | C5 | NAG | C | 367 | 41.511 | 37.296 | 28.348 | 1.00 | 249.69 |
| 1530 | O5 | NAG | C | 367 | 40.568 | 36.196 | 28.373 | 1.00 | 249.69 |
| 1531 | C6 | NAG | C | 367 | 41.060 | 38.236 | 27.251 | 1.00 | 249.69 |
| 1532 | O6 | NAG | C | 367 | 40.020 | 37.661 | 26.474 | 1.00 | 249.69 |
| 1533 | CB | LYS | A | 4 | 3.684 | 19.933 | 14.932 | 1.00 | 249.69 |
| 1534 | CG | LYS | A | 4 | 2.729 | 21.022 | 14.456 | 1.00 | 249.69 |
| 1535 | CD | LYS | A | 4 | 2.217 | 21.880 | 15.610 | 1.00 | 249.69 |
| 1536 | CE | LYS | A | 4 | 1.292 | 22.987 | 15.108 | 1.00 | 249.69 |
| 1537 | NZ | LYS | A | 4 | 0.762 | 23.841 | 16.212 | 1.00 | 249.69 |
| 1538 | C | LYS | A | 4 | 5.030 | 20.019 | 12.832 | 1.00 | 249.22 |
| 1539 | O | LYS | A | 4 | 5.450 | 21.116 | 13.205 | 1.00 | 249.22 |
| 1540 | N | LYS | A | 4 | 5.205 | 18.061 | 14.356 | 1.00 | 249.22 |
| 1541 | CA | LYS | A | 4 | 4.291 | 19.100 | 13.797 | 1.00 | 249.22 |
| 1542 | N | PRO | A | 5 | 5.213 | 19.581 | 11.582 | 1.00 | 249.41 |
| 1543 | CD | PRO | A | 5 | 4.979 | 18.215 | 11.068 | 1.00 | 133.18 |
| 1544 | CA | PRO | A | 5 | 5.912 | 20.398 | 10.589 | 1.00 | 249.41 |
| 1545 | CB | PRO | A | 5 | 6.459 | 19.360 | 9.606 | 1.00 | 133.18 |
| 1546 | CG | PRO | A | 5 | 5.376 | 18.335 | 9.599 | 1.00 | 133.18 |
| 1547 | C | PRO | A | 5 | 4.969 | 21.407 | 9.927 | 1.00 | 249.41 |
| 1548 | O | PRO | A | 5 | 3.754 | 21.219 | 9.927 | 1.00 | 249.41 |
| 1549 | N | LYS | A | 6 | 5.529 | 22.477 | 9.377 | 1.00 | 196.60 |
| 1550 | CA | LYS | A | 6 | 4.724 | 23.489 | 8.709 | 1.00 | 196.60 |
| 1551 | CB | LYS | A | 6 | 4.429 | 24.652 | 9.660 | 1.00 | 249.69 |
| 1552 | CG | LYS | A | 6 | 3.524 | 25.719 | 9.050 | 1.00 | 249.69 |
| 1553 | CD | LYS | A | 6 | 3.113 | 26.783 | 10.067 | 1.00 | 249.69 |
| 1554 | CE | LYS | A | 6 | 2.180 | 27.812 | 9.436 | 1.00 | 249.69 |
| 1555 | NZ | LYS | A | 6 | 1.664 | 28.800 | 10.424 | 1.00 | 249.69 |
| 1556 | C | LYS | A | 6 | 5.433 | 24.000 | 7.458 | 1.00 | 196.60 |
| 1557 | O | LYS | A | 6 | 6.478 | 24.664 | 7.539 | 1.00 | 196.60 |
| 1558 | N | VAL | A | 7 | 4.850 | 23.695 | 6.304 | 1.00 | 192.34 |
| 1559 | CA | VAL | A | 7 | 5.416 | 24.094 | 5.029 | 1.00 | 192.34 |
| 1560 | CB | VAL | A | 7 | 4.656 | 23.429 | 3.870 | 1.00 | 160.27 |
| 1561 | CG1 | VAL | A | 7 | 5.470 | 23.549 | 2.587 | 1.00 | 160.27 |
| 1562 | CG2 | VAL | A | 7 | 4.363 | 21.983 | 4.195 | 1.00 | 160.27 |
| 1563 | C | VAL | A | 7 | 5.403 | 25.607 | 4.807 | 1.00 | 192.34 |
| 1564 | O | VAL | A | 7 | 4.350 | 26.253 | 4.868 | 1.00 | 192.34 |
| 1565 | N | SER | A | 8 | 6.582 | 26.165 | 4.544 | 1.00 | 184.23 |
| 1566 | CA | SER | A | 8 | 6.726 | 27.594 | 4.284 | 1.00 | 184.23 |
| 1567 | CB | SER | A | 8 | 7.897 | 28.148 | 5.099 | 1.00 | 230.08 |
| 1568 | OG | SER | A | 8 | 9.063 | 27.354 | 4.945 | 1.00 | 230.08 |
| 1569 | C | SER | A | 8 | 6.978 | 27.814 | 2.789 | 1.00 | 184.23 |
| 1570 | O | SER | A | 8 | 7.389 | 26.889 | 2.087 | 1.00 | 184.23 |
| 1571 | N | LEU | A | 9 | 6.726 | 29.025 | 2.297 | 1.00 | 167.11 |
| 1572 | CA | LEU | A | 9 | 6.948 | 29.312 | 0.880 | 1.00 | 167.11 |
| 1573 | CB | LEU | A | 9 | 5.626 | 29.535 | 0.147 | 1.00 | 178.21 |
| 1574 | CG | LEU | A | 9 | 4.541 | 28.451 | 0.105 | 1.00 | 178.21 |
| 1575 | CD1 | LEU | A | 9 | 3.549 | 28.821 | −0.980 | 1.00 | 178.21 |
| 1576 | CD2 | LEU | A | 9 | 5.128 | 27.083 | −0.188 | 1.00 | 178.21 |
| 1577 | C | LEU | A | 9 | 7.817 | 30.533 | 0.666 | 1.00 | 167.11 |
| 1578 | O | LEU | A | 9 | 7.946 | 31.373 | 1.552 | 1.00 | 167.11 |
| 1579 | N | ASN | A | 10 | 8.405 | 30.629 | −0.522 | 1.00 | 147.32 |
| 1580 | CA | ASN | A | 10 | 9.260 | 31.769 | −0.855 | 1.00 | 147.32 |
| 1581 | CB | ASN | A | 10 | 10.634 | 31.610 | −0.219 | 1.00 | 249.69 |
| 1582 | CG | ASN | A | 10 | 11.421 | 32.902 | −0.234 | 1.00 | 249.69 |
| 1583 | OD1 | ASN | A | 10 | 11.028 | 33.886 | 0.395 | 1.00 | 249.69 |
| 1584 | ND2 | ASN | A | 10 | 12.534 | 32.911 | −0.959 | 1.00 | 249.69 |
| 1585 | C | ASN | A | 10 | 9.396 | 31.902 | −2.374 | 1.00 | 147.32 |
| 1586 | O | ASN | A | 10 | 10.037 | 31.073 | −3.022 | 1.00 | 147.32 |
| 1587 | N | PRO | A | 11 | 8.851 | 32.979 | −2.953 | 1.00 | 237.62 |
| 1588 | CD | PRO | A | 11 | 8.944 | 33.177 | −4.413 | 1.00 | 161.80 |
| 1589 | CA | PRO | A | 11 | 8.057 | 34.058 | −2.348 | 1.00 | 237.62 |
| 1590 | CB | PRO | A | 11 | 7.554 | 34.834 | −3.552 | 1.00 | 161.80 |
| 1591 | CG | PRO | A | 11 | 8.646 | 34.638 | −4.548 | 1.00 | 161.80 |
| 1592 | C | PRO | A | 11 | 6.921 | 33.599 | −1.438 | 1.00 | 237.62 |
| 1593 | O | PRO | A | 11 | 6.554 | 32.428 | −1.435 | 1.00 | 237.62 |
| 1594 | N | PRO | A | 12 | 6.338 | 34.529 | −0.662 | 1.00 | 147.54 |
| 1595 | CD | PRO | A | 12 | 6.781 | 35.905 | −0.441 | 1.00 | 140.19 |
| 1596 | CA | PRO | A | 12 | 5.229 | 34.189 | 0.236 | 1.00 | 147.54 |
| 1597 | CB | PRO | A | 12 | 5.107 | 35.433 | 1.112 | 1.00 | 140.19 |
| 1598 | CG | PRO | A | 12 | 6.465 | 36.081 | 1.016 | 1.00 | 140.19 |
| 1599 | C | PRO | A | 12 | 3.967 | 33.943 | −0.572 | 1.00 | 147.54 |
| 1600 | O | PRO | A | 12 | 3.063 | 33.202 | −0.148 | 1.00 | 147.54 |
| 1601 | N | TRP | A | 13 | 3.929 | 34.576 | −1.744 | 1.00 | 165.94 |
| 1602 | CA | TRP | A | 13 | 2.824 | 34.492 | −2.698 | 1.00 | 165.94 |
| 1603 | CB | TRP | A | 13 | 3.247 | 35.209 | −3.968 | 1.00 | 139.27 |
| 1604 | CG | TRP | A | 13 | 3.825 | 36.552 | −3.699 | 1.00 | 139.27 |
| 1605 | CD2 | TRP | A | 13 | 3.455 | 37.438 | −2.648 | 1.00 | 139.27 |
| 1606 | CE2 | TRP | A | 13 | 4.233 | 38.603 | −2.790 | 1.00 | 139.27 |
| 1607 | CE3 | TRP | A | 13 | 2.546 | 37.363 | −1.594 | 1.00 | 139.27 |
| 1608 | CD1 | TRP | A | 13 | 4.784 | 37.201 | −4.428 | 1.00 | 139.27 |
| 1609 | NE1 | TRP | A | 13 | 5.035 | 38.438 | −3.891 | 1.00 | 139.27 |
| 1610 | CZ2 | TRP | A | 13 | 4.122 | 39.682 | −1.917 | 1.00 | 139.27 |
| 1611 | CZ3 | TRP | A | 13 | 2.433 | 38.434 | −0.731 | 1.00 | 139.27 |
| 1612 | CH2 | TRP | A | 13 | 3.218 | 39.577 | −0.892 | 1.00 | 139.27 |
| 1613 | C | TRP | A | 13 | 2.428 | 33.061 | −3.048 | 1.00 | 165.94 |
| 1614 | O | TRP | A | 13 | 3.219 | 32.342 | −3.663 | 1.00 | 165.94 |
| 1615 | N | ASN | A | 14 | 1.213 | 32.652 | −2.689 | 1.00 | 109.00 |
| 1616 | CA | ASN | A | 14 | 0.782 | 31.293 | −2.990 | 1.00 | 109.00 |
| 1617 | CB | ASN | A | 14 | 0.167 | 30.660 | −1.746 | 1.00 | 167.27 |
| 1618 | CG | ASN | A | 14 | −1.091 | 31.352 | −1.312 | 1.00 | 167.27 |
| 1619 | OD1 | ASN | A | 14 | −1.088 | 32.556 | −1.050 | 1.00 | 167.27 |
| 1620 | ND2 | ASN | A | 14 | −2.188 | 30.596 | −1.234 | 1.00 | 167.27 |
| 1621 | C | ASN | A | 14 | −0.200 | 31.224 | −4.164 | 1.00 | 109.00 |
| 1622 | O | ASN | A | 14 | −0.981 | 30.275 | −4.295 | 1.00 | 109.00 |
| 1623 | N | ARG | A | 15 | −0.153 | 32.255 | −5.006 | 1.00 | 160.32 |
| 1624 | CA | ARG | A | 15 | −0.977 | 32.384 | −6.220 | 1.00 | 160.32 |
| 1625 | CB | ARG | A | 15 | −2.094 | 33.426 | −6.042 | 1.00 | 119.95 |
| 1626 | CG | ARG | A | 15 | −2.974 | 33.286 | −4.790 | 1.00 | 119.95 |
| 1627 | CD | ARG | A | 15 | −4.127 | 34.296 | −4.834 | 1.00 | 119.95 |
| 1628 | NE | ARG | A | 15 | −5.205 | 33.881 | −5.737 | 1.00 | 119.95 |
| 1629 | CZ | ARG | A | 15 | −5.920 | 34.709 | −6.500 | 1.00 | 119.95 |
| 1630 | NH1 | ARG | A | 15 | −5.674 | 36.015 | −6.492 | 1.00 | 119.95 |
| 1631 | NH2 | ARG | A | 15 | −6.894 | 34.239 | −7.267 | 1.00 | 119.95 |
| 1632 | C | ARG | A | 15 | 0.012 | 32.914 | −7.260 | 1.00 | 160.32 |
| 1633 | O | ARG | A | 15 | 0.338 | 34.098 | −7.259 | 1.00 | 160.32 |
| 1634 | N | ILE | A | 16 | 0.490 | 32.054 | −8.148 | 1.00 | 135.68 |
| 1635 | CA | ILE | A | 16 | 1.479 | 32.491 | −9.124 | 1.00 | 135.68 |
| 1636 | CB | ILE | A | 16 | 2.803 | 31.783 | −8.904 | 1.00 | 134.22 |
| 1637 | CG2 | ILE | A | 16 | 3.532 | 32.401 | −7.704 | 1.00 | 134.22 |
| 1638 | CG1 | ILE | A | 16 | 2.534 | 30.272 | −8.762 | 1.00 | 134.22 |
| 1639 | CD1 | ILE | A | 16 | 3.763 | 29.404 | −8.762 | 1.00 | 134.22 |
| 1640 | C | ILE | A | 16 | 1.141 | 32.283 | −10.581 | 1.00 | 135.68 |
| 1641 | O | ILE | A | 16 | 0.358 | 31.408 | −10.938 | 1.00 | 135.68 |
| 1642 | N | PHE | A | 17 | 1.774 | 33.090 | −11.425 | 1.00 | 145.71 |
| 1643 | CA | PHE | A | 17 | 1.589 | 33.015 | −12.870 | 1.00 | 145.71 |
| 1644 | CB | PHE | A | 17 | 2.211 | 34.246 | −13.547 | 1.00 | 146.10 |
| 1645 | CG | PHE | A | 17 | 1.276 | 35.401 | −13.687 | 1.00 | 146.10 |
| 1646 | CD1 | PHE | A | 17 | 1.752 | 36.702 | −13.601 | 1.00 | 146.10 |
| 1647 | CD2 | PHE | A | 17 | −0.067 | 35.195 | −13.957 | 1.00 | 146.10 |
| 1648 | CE1 | PHE | A | 17 | 0.901 | 37.798 | −13.781 | 1.00 | 146.10 |
| 1649 | CE2 | PHE | A | 17 | −0.927 | 36.273 | −14.142 | 1.00 | 146.10 |
| 1650 | CZ | PHE | A | 17 | −0.437 | 37.586 | −14.054 | 1.00 | 146.10 |
| 1651 | C | PHE | A | 17 | 2.240 | 31.744 | −13.417 | 1.00 | 145.71 |
| 1652 | O | PHE | A | 17 | 2.882 | 30.991 | −12.692 | 1.00 | 145.71 |
| 1653 | N | LYS | A | 18 | 2.074 | 31.534 | −14.713 | 1.00 | 190.00 |
| 1654 | CA | LYS | A | 18 | 2.625 | 30.380 | −15.402 | 1.00 | 190.00 |
| 1655 | CB | LYS | A | 18 | 1.798 | 30.115 | −16.669 | 1.00 | 249.19 |
| 1656 | CG | LYS | A | 18 | 2.212 | 28.904 | −17.483 | 1.00 | 249.19 |
| 1657 | CD | LYS | A | 18 | 1.206 | 28.655 | −18.601 | 1.00 | 249.19 |
| 1658 | CE | LYS | A | 18 | 1.619 | 27.498 | −19.493 | 1.00 | 249.19 |
| 1659 | NZ | LYS | A | 18 | 2.837 | 27.814 | −20.292 | 1.00 | 249.19 |
| 1660 | C | LYS | A | 18 | 4.101 | 30.602 | −15.765 | 1.00 | 190.00 |
| 1661 | O | LYS | A | 18 | 4.472 | 31.613 | −16.368 | 1.00 | 190.00 |
| 1662 | N | GLY | A | 19 | 4.945 | 29.648 | −15.390 | 1.00 | 217.86 |
| 1663 | CA | GLY | A | 19 | 6.356 | 29.759 | −15.698 | 1.00 | 217.86 |
| 1664 | C | GLY | A | 19 | 7.219 | 30.324 | −14.582 | 1.00 | 217.86 |
| 1665 | O | GLY | A | 19 | 6.449 | 30.261 | −14.675 | 1.00 | 217.86 |
| 1666 | N | GLU | A | 20 | 6.593 | 30.874 | −13.537 | 1.00 | 170.23 |
| 1667 | CA | GLU | A | 20 | 7.330 | 31.452 | −12.399 | 1.00 | 170.23 |
| 1668 | CB | GLU | A | 20 | 6.435 | 32.409 | −11.611 | 1.00 | 186.84 |
| 1669 | CG | GLU | A | 20 | 5.663 | 33.418 | −12.440 | 1.00 | 186.84 |
| 1670 | CD | GLU | A | 20 | 4.890 | 34.410 | −11.578 | 1.00 | 186.84 |
| 1671 | OE1 | GLU | A | 20 | 4.121 | 33.972 | −10.688 | 1.00 | 186.84 |
| 1672 | OE2 | GLU | A | 20 | 5.053 | 35.633 | −11.793 | 1.00 | 186.84 |
| 1673 | C | GLU | A | 20 | 7.823 | 30.341 | −11.456 | 1.00 | 170.23 |
| 1674 | O | GLU | A | 20 | 7.274 | 29.232 | −11.466 | 1.00 | 170.23 |
| 1675 | N | ASN | A | 21 | 8.838 | 30.634 | −10.636 | 1.00 | 187.01 |
| 1676 | CA | ASN | A | 21 | 9.372 | 29.622 | −9.707 | 1.00 | 187.01 |
| 1677 | CB | ASN | A | 21 | 10.888 | 29.456 | −9.859 | 1.00 | 249.69 |
| 1678 | CG | ASN | A | 21 | 11.371 | 29.621 | −11.291 | 1.00 | 249.69 |
| 1679 | OD1 | ASN | A | 21 | 10.828 | 29.039 | −12.233 | 1.00 | 249.69 |
| 1680 | ND2 | ASN | A | 21 | 12.423 | 30.420 | −11.435 | 1.00 | 249.69 |
| 1681 | C | ASN | A | 21 | 9.087 | 29.907 | −8.230 | 1.00 | 187.01 |
| 1682 | O | ASN | A | 21 | 9.136 | 31.054 | −7.786 | 1.00 | 187.01 |
| 1683 | N | VAL | A | 22 | 8.816 | 28.842 | −7.477 | 1.00 | 223.09 |
| 1684 | CA | VAL | A | 22 | 8.516 | 28.936 | −6.050 | 1.00 | 223.09 |
| 1685 | CB | VAL | A | 22 | 6.995 | 28.809 | −5.785 | 1.00 | 159.07 |
| 1686 | CG1 | VAL | A | 22 | 6.530 | 27.388 | −6.039 | 1.00 | 159.07 |
| 1687 | CG2 | VAL | A | 22 | 6.680 | 29.212 | −4.363 | 1.00 | 159.07 |
| 1688 | C | VAL | A | 22 | 9.228 | 27.825 | −5.280 | 1.00 | 223.09 |
| 1689 | O | VAL | A | 22 | 9.418 | 26.731 | −5.801 | 1.00 | 223.09 |
| 1690 | N | THR | A | 23 | 9.600 | 28.102 | −4.033 | 1.00 | 162.43 |
| 1691 | CA | THR | A | 23 | 10.307 | 27.125 | −3.197 | 1.00 | 162.43 |
| 1692 | CB | THR | A | 23 | 11.677 | 27.680 | −2.758 | 1.00 | 218.62 |
| 1693 | OG1 | THR | A | 23 | 12.384 | 28.165 | −3.905 | 1.00 | 218.62 |
| 1694 | CG2 | THR | A | 23 | 12.498 | 26.594 | −2.071 | 1.00 | 218.62 |
| 1695 | C | THR | A | 23 | 9.549 | 26.715 | −1.924 | 1.00 | 162.43 |
| 1696 | O | THR | A | 23 | 9.185 | 27.571 | −1.114 | 1.00 | 162.43 |
| 1697 | N | LEU | A | 24 | 9.337 | 25.410 | −1.736 | 1.00 | 159.52 |
| 1698 | CA | LEU | A | 24 | 8.635 | 24.916 | −0.549 | 1.00 | 159.52 |
| 1699 | CB | LEU | A | 24 | 7.593 | 23.860 | −0.923 | 1.00 | 128.43 |
| 1700 | CG | LEU | A | 24 | 6.845 | 23.919 | −2.252 | 1.00 | 128.43 |
| 1701 | CD1 | LEU | A | 24 | 5.664 | 22.963 | −2.175 | 1.00 | 128.43 |
| 1702 | CD2 | LEU | A | 24 | 6.352 | 25.322 | −2.557 | 1.00 | 128.43 |
| 1703 | C | LEU | A | 24 | 9.600 | 24.299 | 0.464 | 1.00 | 159.52 |
| 1704 | O | LEU | A | 24 | 10.111 | 23.201 | 0.247 | 1.00 | 159.52 |
| 1705 | N | THR | A | 25 | 9.827 | 24.999 | 1.574 | 1.00 | 201.17 |
| 1706 | CA | THR | A | 25 | 10.722 | 24.533 | 2.637 | 1.00 | 201.17 |
| 1707 | CB | THR | A | 25 | 11.524 | 25.712 | 3.227 | 1.00 | 221.92 |
| 1708 | OG1 | THR | A | 25 | 12.249 | 26.363 | 2.178 | 1.00 | 221.92 |
| 1709 | CG2 | THR | A | 25 | 12.501 | 25.225 | 4.293 | 1.00 | 221.92 |
| 1710 | C | THR | A | 25 | 9.919 | 23.875 | 3.767 | 1.00 | 201.17 |
| 1711 | O | THR | A | 25 | 8.912 | 24.429 | 4.215 | 1.00 | 201.17 |
| 1712 | N | CYS | A | 26 | 10.363 | 22.707 | 4.232 | 1.00 | 178.89 |
| 1713 | CA | CYS | A | 26 | 9.668 | 21.995 | 5.311 | 1.00 | 178.89 |
| 1714 | C | CYS | A | 26 | 10.061 | 22.556 | 6.672 | 1.00 | 178.89 |
| 1715 | O | CYS | A | 26 | 11.220 | 22.904 | 6.885 | 1.00 | 178.89 |
| 1716 | CB | CYS | A | 26 | 9.989 | 20.504 | 5.257 | 1.00 | 171.78 |
| 1717 | SG | CYS | A | 26 | 8.970 | 19.467 | 6.366 | 1.00 | 171.78 |
| 1718 | N | ASN | A | 27 | 9.095 | 22.623 | 7.589 | 1.00 | 234.74 |
| 1719 | CA | ASN | A | 27 | 9.307 | 23.180 | 8.929 | 1.00 | 234.74 |
| 1720 | CB | ASN | A | 27 | 8.591 | 22.337 | 9.987 | 1.00 | 249.69 |
| 1721 | CG | ASN | A | 27 | 8.555 | 23.020 | 11.351 | 1.00 | 249.69 |
| 1722 | OD1 | ASN | A | 27 | 8.194 | 24.193 | 11.463 | 1.00 | 249.69 |
| 1723 | ND2 | ASN | A | 27 | 8.928 | 22.284 | 12.395 | 1.00 | 249.69 |
| 1724 | C | ASN | A | 27 | 10.772 | 23.362 | 9.323 | 1.00 | 234.74 |
| 1725 | O | ASN | A | 27 | 11.425 | 22.453 | 9.832 | 1.00 | 234.74 |
| 1726 | N | GLY | A | 28 | 11.267 | 24.569 | 9.076 | 1.00 | 249.69 |
| 1727 | CA | GLY | A | 28 | 12.641 | 24.927 | 9.380 | 1.00 | 249.69 |
| 1728 | C | GLY | A | 28 | 12.886 | 26.299 | 8.768 | 1.00 | 249.69 |
| 1729 | O | GLY | A | 28 | 12.749 | 26.475 | 7.551 | 1.00 | 249.69 |
| 1730 | N | ASN | A | 29 | 13.240 | 27.275 | 9.600 | 1.00 | 249.69 |
| 1731 | CA | ASN | A | 29 | 13.468 | 28.641 | 9.124 | 1.00 | 249.69 |
| 1732 | CB | ASN | A | 29 | 13.452 | 29.617 | 10.321 | 1.00 | 249.69 |
| 1733 | CG | ASN | A | 29 | 13.401 | 31.093 | 9.896 | 1.00 | 249.69 |
| 1734 | OD1 | ASN | A | 29 | 13.221 | 31.413 | 8.716 | 1.00 | 249.69 |
| 1735 | ND2 | ASN | A | 29 | 13.548 | 31.993 | 10.868 | 1.00 | 249.69 |
| 1736 | C | ASN | A | 29 | 14.761 | 28.813 | 8.314 | 1.00 | 249.69 |
| 1737 | O | ASN | A | 29 | 14.726 | 29.331 | 7.190 | 1.00 | 249.69 |
| 1738 | N | ASN | A | 30 | 15.890 | 28.365 | 8.861 | 1.00 | 249.69 |
| 1739 | CA | ASN | A | 30 | 17.157 | 28.533 | 8.158 | 1.00 | 249.69 |
| 1740 | CB | ASN | A | 30 | 18.002 | 29.581 | 8.895 | 1.00 | 249.69 |
| 1741 | CG | ASN | A | 30 | 17.349 | 30.959 | 8.911 | 1.00 | 249.69 |
| 1742 | OD1 | ASN | A | 30 | 17.266 | 31.607 | 9.960 | 1.00 | 249.69 |
| 1743 | ND2 | ASN | A | 30 | 16.888 | 31.416 | 7.744 | 1.00 | 249.69 |
| 1744 | C | ASN | A | 30 | 17.985 | 27.267 | 7.919 | 1.00 | 249.69 |
| 1745 | O | ASN | A | 30 | 18.147 | 26.838 | 6.774 | 1.00 | 249.69 |
| 1746 | N | PHE | A | −31 | 18.512 | 26.668 | 8.987 | 1.00 | 249.69 |
| 1747 | CA | PHE | A | 31 | 19.345 | 25.474 | 8.836 | 1.00 | 249.69 |
| 1748 | CB | PHE | A | 31 | 20.748 | 25.733 | 9.416 | 1.00 | 249.69 |
| 1749 | CG | PHE | A | 31 | 21.429 | 26.957 | 8.841 | 1.00 | 249.69 |
| 1750 | CD1 | PHE | A | 31 | 21.106 | 28.234 | 9.300 | 1.00 | 249.69 |
| 1751 | CD2 | PHE | A | 31 | 22.372 | 26.835 | 7.822 | 1.00 | 249.69 |
| 1752 | CE1 | PHE | A | 31 | 21.706 | 29.372 | 8.749 | 1.00 | 249.69 |
| 1753 | CE2 | PHE | A | 31 | 22.978 | 27.971 | 7.263 | 1.00 | 249.69 |
| 1754 | CZ | PHE | A | 31 | 22.644 | 29.238 | 7.730 | 1.00 | 249.69 |
| 1755 | C | PHE | A | 31 | 18.752 | 24.200 | 9.450 | 1.00 | 249.69 |
| 1756 | O | PHE | A | 31 | 18.444 | 24.144 | 10.647 | 1.00 | 249.69 |
| 1757 | N | PHE | A | 32 | 18.608 | 23.175 | 8.608 | 1.00 | 249.69 |
| 1758 | CA | PHE | A | 32 | 18.052 | 21.879 | 9.017 | 1.00 | 249.69 |
| 1759 | CB | PHE | A | 32 | 16.789 | 21.579 | 8.201 | 1.00 | 249.69 |
| 1760 | CG | PHE | A | 32 | 15.943 | 20.469 | 8.766 | 1.00 | 249.69 |
| 1761 | CD1 | PHE | A | 32 | 15.293 | 20.621 | 9.991 | 1.00 | 249.69 |
| 1762 | CD2 | PHE | A | 32 | 15.785 | 19.274 | 8.064 | 1.00 | 249.69 |
| 1763 | CE1 | PHE | A | 32 | 14.496 | 19.598 | 10.509 | 1.00 | 249.69 |
| 1764 | CE2 | PHE | A | 32 | 14.990 | 18.245 | 8.573 | 1.00 | 249.69 |
| 1765 | CZ | PHE | A | 32 | 14.345 | 18.408 | 9.798 | 1.00 | 249.69 |
| 1766 | C | PHE | A | 32 | 19.088 | 20.757 | 8.821 | 1.00 | 249.69 |
| 1767 | O | PHE | A | 32 | 20.125 | 20.964 | 8.170 | 1.00 | 249.69 |
| 1768 | N | GLU | A | 33 | 18.798 | 19.569 | 9.358 | 1.00 | 231.29 |
| 1769 | CA | GLU | A | 33 | 19.741 | 18.455 | 9.270 | 1.00 | 231.29 |
| 1770 | CB | GLU | A | 33 | 20.145 | 18.036 | 10.688 | 1.00 | 249.69 |
| 1771 | CG | GLU | A | 33 | 21.430 | 17.234 | 10.751 | 1.00 | 249.69 |
| 1772 | CD | GLU | A | 33 | 22.544 | 17.870 | 9.925 | 1.00 | 249.69 |
| 1773 | OE1 | GLU | A | 33 | 22.781 | 19.095 | 10.061 | 1.00 | 249.69 |
| 1774 | OE2 | GLU | A | 33 | 23.193 | 17.142 | 9.140 | 1.00 | 249.69 |
| 1775 | C | GLU | A | 33 | 19.334 | 17.212 | 8.477 | 1.00 | 231.29 |
| 1776 | O | GLU | A | 33 | 20.088 | 16.746 | 7.624 | 1.00 | 231.29 |
| 1777 | N | VAL | A | 34 | 18.156 | 16.671 | 8.765 | 1.00 | 249.69 |
| 1778 | CA | VAL | A | 34 | 17.677 | 15.462 | 8.096 | 1.00 | 249.69 |
| 1779 | CB | VAL | A | 34 | 16.288 | 15.045 | 8.664 | 1.00 | 206.86 |
| 1780 | CG1 | VAL | A | 34 | 15.809 | 13.765 | 8.012 | 1.00 | 206.86 |
| 1781 | CG2 | VAL | A | 34 | 16.382 | 14.858 | 10.166 | 1.00 | 206.86 |
| 1782 | C | VAL | A | 34 | 17.599 | 15.536 | 6.560 | 1.00 | 249.69 |
| 1783 | O | VAL | A | 34 | 17.381 | 16.608 | 5.977 | 1.00 | 249.69 |
| 1784 | N | SER | A | 35 | 17.793 | 14.378 | 5.920 | 1.00 | 249.69 |
| 1785 | CA | SER | A | 35 | 17.744 | 14.245 | 4.458 | 1.00 | 249.69 |
| 1786 | CB | SER | A | 35 | 18.968 | 13.478 | 3.941 | 1.00 | 177.13 |
| 1787 | OG | SER | A | 35 | 18.874 | 12.099 | 4.268 | 1.00 | 177.13 |
| 1788 | C | SER | A | 35 | 16.483 | 13.467 | 4.082 | 1.00 | 249.69 |
| 1789 | O | SER | A | 35 | 16.208 | 13.245 | 2.902 | 1.00 | 249.69 |
| 1790 | N | SER | A | 36 | 15.739 | 13.038 | 5.100 | 1.00 | 238.60 |
| 1791 | CA | SER | A | 36 | 14.506 | 12.290 | 4.902 | 1.00 | 238.60 |
| 1792 | CB | SER | A | 36 | 14.437 | 11.091 | 5.862 | 1.00 | 249.69 |
| 1793 | OG | SER | A | 36 | 14.205 | 11.498 | 7.203 | 1.00 | 249.69 |
| 1794 | C | SER | A | 36 | 13.298 | 13.200 | 5.121 | 1.00 | 238.60 |
| 1795 | O | SER | A | 36 | 12.807 | 13.368 | 6.238 | 1.00 | 238.60 |
| 1796 | N | THR | A | 37 | 12.835 | 13.795 | 4.030 | 1.00 | 223.58 |
| 1797 | CA | THR | A | 37 | 11.686 | 14.678 | 4.061 | 1.00 | 223.58 |
| 1798 | CB | THR | A | 37 | 12.108 | 16.135 | 3.751 | 1.00 | 216.59 |
| 1799 | OG1 | THR | A | 37 | 13.071 | 16.572 | 4.723 | 1.00 | 216.59 |
| 1800 | CG2 | THR | A | 37 | 10.904 | 17.061 | 3.786 | 1.00 | 216.59 |
| 1801 | C | THR | A | 37 | 10.706 | 14.165 | 3.004 | 1.00 | 223.58 |
| 1802 | O | THR | A | 37 | 11.104 | 13.773 | 1.901 | 1.00 | 223.58 |
| 1803 | N | LYS | A | 38 | 9.425 | 14.148 | 3.349 | 1.00 | 249.69 |
| 1804 | CA | LYS | A | 38 | 8.410 | 13.655 | 2.430 | 1.00 | 249.69 |
| 1805 | CB | LYS | A | 38 | 7.490 | 12.670 | 3.166 | 1.00 | 249.69 |
| 1806 | CG | LYS | A | 38 | 8.232 | 11.473 | 3.770 | 1.00 | 249.69 |
| 1807 | CD | LYS | A | 38 | 7.296 | 10.511 | 4.515 | 1.00 | 249.69 |
| 1808 | CE | LYS | A | 38 | 8.060 | 9.293 | 5.053 | 1.00 | 249.69 |
| 1809 | NZ | LYS | A | 38 | 7.181 | 8.326 | 5.770 | 1.00 | 249.69 |
| 1810 | C | LYS | A | 38 | 7.588 | 14.782 | 1.806 | 1.00 | 249.69 |
| 1811 | O | LYS | A | 38 | 7.301 | 15.793 | 2.456 | 1.00 | 249.69 |
| 1812 | N | TRP | A | 39 | 7.229 | 14.611 | 0.536 | 1.00 | 201.19 |
| 1813 | CA | TRP | A | 39 | 6.425 | 15.604 | −0.171 | 1.00 | 201.19 |
| 1814 | CB | TRP | A | 39 | 7.256 | 16.294 | −1.250 | 1.00 | 173.49 |
| 1815 | CG | TRP | A | 39 | 8.384 | 17.170 | −0.741 | 1.00 | 173.49 |
| 1816 | CD2 | TRP | A | 39 | 8.282 | 18.328 | 0.122 | 1.00 | 173.49 |
| 1817 | CE2 | TRP | A | 39 | 9.574 | 18.892 | 0.223 | 1.00 | 173.49 |
| 1818 | CE3 | TRP | A | 39 | 7.223 | 18.950 | 0.812 | 1.00 | 173.49 |
| 1819 | CD1 | TRP | A | 39 | 9.703 | 17.079 | −1.099 | 1.00 | 173.49 |
| 1820 | NE1 | TRP | A | 39 | 10.418 | 18.112 | −0.528 | 1.00 | 173.49 |
| 1821 | CZ2 | TRP | A | 39 | 9.836 | 20.031 | 0.972 | 1.00 | 173.49 |
| 1822 | CZ3 | TRP | A | 39 | 7.489 | 20.083 | 1.554 | 1.00 | 173.49 |
| 1823 | CH2 | TRP | A | 39 | 8.785 | 20.611 | 1.629 | 1.00 | 173.49 |
| 1824 | C | TRP | A | 39 | 5.263 | 14.870 | −0.821 | 1.00 | 201.19 |
| 1825 | O | TRP | A | 39 | 5.473 | 13.844 | −1.463 | 1.00 | 201.19 |
| 1826 | N | PHE | A | 40 | 4.045 | 15.385 | −0.655 | 1.00 | 233.06 |
| 1827 | CA | PHE | A | 40 | 2.875 | 14.733 | −1.231 | 1.00 | 233.06 |
| 1828 | CB | PHE | A | 40 | 1.983 | 14.154 | −0.122 | 1.00 | 249.42 |
| 1829 | CG | PHE | A | 40 | 2.671 | 13.151 | 0.775 | 1.00 | 249.42 |
| 1830 | CD1 | PHE | A | 40 | 3.484 | 13.580 | 1.820 | 1.00 | 249.42 |
| 1831 | CD2 | PHE | A | 40 | 2.482 | 11.778 | 0.592 | 1.00 | 249.42 |
| 1832 | CE1 | PHE | A | 40 | 4.098 | 12.658 | 2.674 | 1.00 | 249.42 |
| 1833 | CE2 | PHE | A | 40 | 3.089 | 10.854 | 1.435 | 1.00 | 249.42 |
| 1834 | CZ | PHE | A | 40 | 3.899 | 11.294 | 2.479 | 1.00 | 249.42 |
| 1835 | C | PHE | A | 40 | 2.023 | 15.621 | −2.139 | 1.00 | 233.06 |
| 1836 | O | PHE | A | 40 | 0.945 | 16.063 | −1.744 | 1.00 | 233.06 |
| 1837 | N | HIS | A | 41 | 2.506 | 15.858 | −3.358 | 1.00 | 146.58 |
| 1838 | CA | HIS | A | 41 | 1.787 | 16.676 | −4.340 | 1.00 | 146.58 |
| 1839 | CB | HIS | A | 41 | 2.663 | 16.905 | −5.569 | 1.00 | 196.00 |
| 1840 | CG | HIS | A | 41 | 2.012 | 17.747 | −6.619 | 1.00 | 196.00 |
| 1841 | CD2 | HIS | A | 41 | 2.035 | 17.661 | −7.971 | 1.00 | 196.00 |
| 1842 | ND1 | HIS | A | 41 | 1.259 | 18.864 | −6.322 | 1.00 | 196.00 |
| 1843 | CE1 | HIS | A | 41 | 0.849 | 19.429 | −7.441 | 1.00 | 196.00 |
| 1844 | NE2 | HIS | A | 41 | 1.308 | 18.719 | −8.457 | 1.00 | 196.00 |
| 1845 | C | HIS | A | 41 | 0.459 | 16.041 | −4.776 | 1.00 | 146.58 |
| 1846 | O | HIS | A | 41 | 0.458 | 15.095 | −5.564 | 1.00 | 146.58 |
| 1847 | N | ASN | A | 42 | −0.660 | 16.586 | −4.280 | 1.00 | 208.40 |
| 1848 | CA | ASN | A | 42 | −2.004 | 16.067 | −4.570 | 1.00 | 208.40 |
| 1849 | CB | ASN | A | 42 | −2.229 | 15.933 | −6.087 | 1.00 | 249.69 |
| 1850 | CG | ASN | A | 42 | −2.538 | 17.270 | −6.763 | 1.00 | 249.69 |
| 1851 | OD1 | ASN | A | 42 | −1.824 | 18.251 | −6.553 | 1.00 | 249.69 |
| 1852 | ND2 | ASN | A | 42 | −3.591 | 17.305 | −7.583 | 1.00 | 249.69 |
| 1853 | C | ASN | A | 42 | −2.173 | 14.703 | −3.887 | 1.00 | 208.40 |
| 1854 | O | ASN | A | 42 | −2.981 | 13.871 | −4.302 | 1.00 | 208.40 |
| 1855 | N | GLY | A | 43 | −1.401 | 14.499 | −2.824 | 1.00 | 249.69 |
| 1856 | CA | GLY | A | 43 | −1.445 | 13.248 | −2.092 | 1.00 | 249.69 |
| 1857 | C | GLY | A | 43 | −0.354 | 12.288 | −2.555 | 1.00 | 249.69 |
| 1858 | O | GLY | A | 43 | 0.302 | 11.620 | −1.744 | 1.00 | 249.69 |
| 1859 | N | SER | A | 44 | −0.158 | 12.222 | −3.870 | 1.00 | 243.81 |
| 1860 | CA | SER | A | 44 | 0.845 | 11.350 | −4.481 | 1.00 | 243.81 |
| 1861 | CB | SER | A | 44 | 0.812 | 11.493 | −6.004 | 1.00 | 249.69 |
| 1862 | OG | SER | A | 44 | −0.450 | 11.141 | −6.535 | 1.00 | 249.69 |
| 1863 | C | SER | A | 44 | 2.250 | 11.676 | −4.002 | 1.00 | 243.81 |
| 1864 | O | SER | A | 44 | 2.714 | 12.806 | −4.162 | 1.00 | 243.81 |
| 1865 | N | LEU | A | 45 | 2.936 | 10.687 | −3.437 | 1.00 | 249.69 |
| 1866 | CA | LEU | A | 45 | 4.294 | 10.912 | −2.958 | 1.00 | 249.69 |
| 1867 | CB | LEU | A | 45 | 4.913 | 9.605 | −2.458 | 1.00 | 240.25 |
| 1868 | CG | LEU | A | 45 | 6.324 | 9.745 | −1.879 | 1.00 | 240.25 |
| 1869 | CD1 | LEU | A | 45 | 6.328 | 10.787 | −0.773 | 1.00 | 240.25 |
| 1870 | CD2 | LEU | A | 45 | 6.798 | 8.405 | −1.351 | 1.00 | 240.25 |
| 1871 | C | LEU | A | 45 | 5.160 | 11.512 | −4.070 | 1.00 | 249.69 |
| 1872 | O | LEU | A | 45 | 4.939 | 11.248 | −5.256 | 1.00 | 249.69 |
| 1873 | N | SER | A | 46 | 6.136 | 12.329 | −3.675 | 1.00 | 216.07 |
| 1874 | CA | SER | A | 46 | 7.028 | 12.988 | −4.621 | 1.00 | 216.07 |
| 1875 | CB | SER | A | 46 | 7.156 | 14.473 | −4.270 | 1.00 | 249.69 |
| 1876 | OG | SER | A | 46 | 7.934 | 15.159 | −5.237 | 1.00 | 249.69 |
| 1877 | C | SER | A | 46 | 8.409 | 12.344 | −4.645 | 1.00 | 216.07 |
| 1878 | O | SER | A | 46 | 8.733 | 11.515 | −3.795 | 1.00 | 216.07 |
| 1879 | N | GLU | A | 47 | 9.223 | 12.753 | −5.616 | 1.00 | 204.74 |
| 1880 | CA | GLU | A | 47 | 10.572 | 12.214 | −5.797 | 1.00 | 204.74 |
| 1881 | CB | GLU | A | 47 | 10.901 | 12.162 | −7.289 | 1.00 | 249.69 |
| 1882 | CG | GLU | A | 47 | 9.973 | 11.256 | −8.078 | 1.00 | 249.69 |
| 1883 | CD | GLU | A | 47 | 10.299 | 11.239 | −9.554 | 1.00 | 249.69 |
| 1884 | OE1 | GLU | A | 47 | 10.185 | 12.307 | −10.203 | 1.00 | 249.69 |
| 1885 | OE2 | GLU | A | 47 | 10.673 | 10.158 | −10.066 | 1.00 | 249.69 |
| 1886 | C | GLU | A | 47 | 11.702 | 12.933 | −5.059 | 1.00 | 204.74 |
| 1887 | O | GLU | A | 47 | 12.819 | 12.424 | −4.995 | 1.00 | 204.74 |
| 1888 | N | GLU | A | 48 | 11.429 | 14.113 | −4.512 | 1.00 | 206.77 |
| 1889 | CA | GLU | A | 48 | 12.459 | 14.833 | −3.780 | 1.00 | 206.77 |
| 1890 | CB | GLU | A | 48 | 12.206 | 16.344 | −3.812 | 1.00 | 249.43 |
| 1891 | CG | GLU | A | 48 | 13.200 | 17.159 | −2.982 | 1.00 | 249.43 |
| 1892 | CD | GLU | A | 48 | 14.627 | 17.087 | −3.507 | 1.00 | 249.43 |
| 1893 | OE1 | GLU | A | 48 | 14.902 | 17.685 | −4.569 | 1.00 | 249.43 |
| 1894 | OE2 | GLU | A | 48 | 15.472 | 16.431 | −2.859 | 1.00 | 249.43 |
| 1895 | C | GLU | A | 48 | 12.492 | 14.344 | −2.335 | 1.00 | 206.77 |
| 1896 | O | GLU | A | 48 | 11.500 | 13.825 | −1.811 | 1.00 | 206.77 |
| 1897 | N | THR | A | 49 | 13.648 | 14.506 | −1.702 | 1.00 | 249.69 |
| 1898 | CA | THR | A | 49 | 13.844 | 14.085 | −0.324 | 1.00 | 249.69 |
| 1899 | CB | THR | A | 49 | 14.806 | 12.890 | −0.252 | 1.00 | 249.53 |
| 1900 | OG1 | THR | A | 49 | 16.030 | 13.218 | −0.928 | 1.00 | 249.53 |
| 1901 | CG2 | THR | A | 49 | 14.174 | 11.671 | −0.909 | 1.00 | 249.53 |
| 1902 | C | THR | A | 49 | 14.417 | 15.232 | 0.500 | 1.00 | 249.69 |
| 1903 | O | THR | A | 49 | 14.224 | 15.294 | 1.716 | 1.00 | 249.69 |
| 1904 | N | ASN | A | 50 | 15.128 | 16.136 | −0.166 | 1.00 | 249.69 |
| 1905 | CA | ASN | A | 50 | 15.710 | 17.294 | 0.504 | 1.00 | 249.69 |
| 1906 | CB | ASN | A | 50 | 16.438 | 18.174 | −0.519 | 1.00 | 232.42 |
| 1907 | CG | ASN | A | 50 | 17.276 | 19.254 | 0.134 | 1.00 | 232.42 |
| 1908 | OD1 | ASN | A | 50 | 17.063 | 19.590 | 1.301 | 1.00 | 232.42 |
| 1909 | ND2 | ASN | A | 50 | 18.219 | 19.813 | −0.615 | 1.00 | 232.42 |
| 1910 | C | ASN | A | 50 | 14.552 | 18.073 | 1.142 | 1.00 | 249.69 |
| 1911 | O | ASN | A | 50 | 13.423 | 18.003 | 0.658 | 1.00 | 249.69 |
| 1912 | N | SER | A | 51 | 14.817 | 18.808 | 2.217 | 1.00 | 181.87 |
| 1913 | CA | SER | A | 51 | 13.759 | 19.568 | 2.873 | 1.00 | 181.87 |
| 1914 | CB | SER | A | 51 | 14.240 | 20.110 | 4.220 | 1.00 | 249.47 |
| 1915 | OG | SER | A | 51 | 15.152 | 21.181 | 4.047 | 1.00 | 249.47 |
| 1916 | C | SER | A | 51 | 13.249 | 20.725 | 2.016 | 1.00 | 181.87 |
| 1917 | O | SER | A | 51 | 12.180 | 21.269 | 2.293 | 1.00 | 181.87 |
| 1918 | N | SER | A | 52 | 14.007 | 21.104 | 0.984 | 1.00 | 193.91 |
| 1919 | CA | SER | A | 52 | 13.606 | 22.199 | 0.087 | 1.00 | 193.91 |
| 1920 | CB | SER | A | 52 | 14.735 | 23.217 | −0.086 | 1.00 | 144.08 |
| 1921 | OG | SER | A | 52 | 15.064 | 23.831 | 1.139 | 1.00 | 144.08 |
| 1922 | C | SER | A | 52 | 13.196 | 21.706 | −1.297 | 1.00 | 193.91 |
| 1923 | O | SER | A | 52 | 14.045 | 21.367 | −2.126 | 1.00 | 193.91 |
| 1924 | N | LEU | A | 53 | 11.890 | 21.680 | −1.539 | 1.00 | 177.42 |
| 1925 | CA | LEU | A | 53 | 11.346 | 21.239 | −2.817 | 1.00 | 177.42 |
| 1926 | CB | LEU | A | 53 | 10.034 | 20.488 | −2.595 | 1.00 | 145.45 |
| 1927 | CG | LEU | A | 53 | 9.082 | 20.340 | −3.785 | 1.00 | 145.45 |
| 1928 | CD1 | LEU | A | 53 | 9.821 | 19.891 | −5.043 | 1.00 | 145.45 |
| 1929 | CD2 | LEU | A | 53 | 7.997 | 19.343 | −3.401 | 1.00 | 145.45 |
| 1930 | C | LEU | A | 53 | 11.108 | 22.423 | −3.737 | 1.00 | 177.42 |
| 1931 | O | LEU | A | 53 | 10.143 | 23.168 | −3.574 | 1.00 | 177.42 |
| 1932 | N | ASN | A | 54 | 11.991 | 22.591 | −4.709 | 1.00 | 220.39 |
| 1933 | CA | ASN | A | 54 | 11.845 | 23.692 | −5.635 | 1.00 | 220.39 |
| 1934 | CB | ASN | A | 54 | 13.187 | 24.045 | −6.254 | 1.00 | 193.36 |
| 1935 | CG | ASN | A | 54 | 14.109 | 24.677 | −5.264 | 1.00 | 193.36 |
| 1936 | OD1 | ASN | A | 54 | 13.746 | 25.644 | −4.602 | 1.00 | 193.36 |
| 1937 | ND2 | ASN | A | 54 | 15.311 | 24.140 | −5.145 | 1.00 | 193.36 |
| 1938 | C | ASN | A | 54 | 10.834 | 23.415 | −6.729 | 1.00 | 220.39 |
| 1939 | O | ASN | A | 54 | 10.486 | 22.267 | −7.009 | 1.00 | 220.39 |
| 1940 | N | ILE | A | 55 | 10.362 | 24.496 | −7.333 | 1.00 | 206.48 |
| 1941 | CA | ILE | A | 55 | 9.393 | 24.451 | −8.415 | 1.00 | 206.48 |
| 1942 | CB | ILE | A | 55 | 7.984 | 24.867 | −7.921 | 1.00 | 168.43 |
| 1943 | CG2 | ILE | A | 55 | 7.135 | 25.353 | −9.080 | 1.00 | 168.43 |
| 1944 | CG1 | ILE | A | 55 | 7.316 | 23.696 | −7.206 | 1.00 | 168.43 |
| 1945 | CD1 | ILE | A | 55 | 5.920 | 24.004 | −6.681 | 1.00 | 168.43 |
| 1946 | C | ILE | A | 55 | 9.877 | 25.442 | −9.459 | 1.00 | 206.48 |
| 1947 | O | ILE | A | 55 | 9.979 | 26.641 | −9.190 | 1.00 | 206.48 |
| 1948 | N | VAL | A | 56 | 10.194 | 24.943 | −10.646 | 1.00 | 242.77 |
| 1949 | CA | VAL | A | 56 | 10.667 | 25.821 | −11.700 | 1.00 | 242.77 |
| 1950 | CB | VAL | A | 56 | 11.790 | 25.165 | −12.499 | 1.00 | 249.69 |
| 1951 | CG1 | VAL | A | 56 | 12.589 | 26.233 | −13.240 | 1.00 | 249.69 |
| 1952 | CG2 | VAL | A | 56 | 12.687 | 24.377 | −11.562 | 1.00 | 249.69 |
| 1953 | C | VAL | A | 56 | 9.511 | 26.168 | −12.624 | 1.00 | 242.77 |
| 1954 | O | VAL | A | 56 | 8.354 | 26.060 | −12.225 | 1.00 | 242.77 |
| 1955 | N | ASN | A | 57 | 9.822 | 26.580 | −13.853 | 1.00 | 177.18 |
| 1956 | CA | ASN | A | 57 | 8.804 | 26.971 | −14.835 | 1.00 | 177.18 |
| 1957 | CB | ASN | A | 57 | 9.265 | 26.619 | −16.250 | 1.00 | 249.69 |
| 1958 | CG | ASN | A | 57 | 10.430 | 27.489 | −16.705 | 1.00 | 249.69 |
| 1959 | OD1 | ASN | A | 57 | 10.372 | 28.721 | −16.617 | 1.00 | 249.69 |
| 1960 | ND2 | ASN | A | 57 | 11.494 | 26.853 | −17.187 | 1.00 | 249.69 |
| 1961 | C | ASN | A | 57 | 7.436 | 26.374 | −14.547 | 1.00 | 177.18 |
| 1962 | o | ASN | A | 57 | 7.105 | 25.264 | −14.964 | 1.00 | 177.18 |
| 1963 | N | ALA | A | 58 | 6.661 | 27.166 | −13.816 | 1.00 | 241.59 |
| 1964 | CA | ALA | A | 58 | 5.322 | 26.838 | −13.362 | 1.00 | 241.59 |
| 1965 | CB | ALA | A | 58 | 4.739 | 28.038 | −12.623 | 1.00 | 177.10 |
| 1966 | C | ALA | A | 58 | 4.339 | 26.363 | −14.416 | 1.00 | 241.59 |
| 1967 | O | ALA | A | 58 | 3.857 | 27.134 | −15.237 | 1.00 | 241.59 |
| 1968 | N | LYS | A | 59 | 4.031 | 25.077 | −14.363 | 1.00 | 126.26 |
| 1969 | CA | LYS | A | 59 | 3.078 | 24.446 | −15.277 | 1.00 | 126.26 |
| 1970 | CB | LYS | A | 59 | 3.620 | 23.088 | −15.761 | 1.00 | 249.69 |
| 1971 | CG | LYS | A | 59 | 4.959 | 23.183 | −16.494 | 1.00 | 249.69 |
| 1972 | CD | LYS | A | 59 | 5.515 | 21.808 | −16.859 | 1.00 | 249.69 |
| 1973 | CE | LYS | A | 59 | 6.883 | 21.939 | −17.528 | 1.00 | 249.69 |
| 1974 | NZ | LYS | A | 59 | 7.458 | 20.619 | −17.910 | 1.00 | 249.69 |
| 1975 | C | LYS | A | 59 | 1.790 | 24.246 | −14.486 | 1.00 | 126.26 |
| 1976 | O | LYS | A | 59 | 1.810 | 23.891 | −13.311 | 1.00 | 126.26 |
| 1977 | N | PHE | A | 60 | 0.672 | 24.490 | −15.139 | 1.00 | 178.77 |
| 1978 | CA | PHE | A | 60 | −0.622 | 24.356 | −14.497 | 1.00 | 178.77 |
| 1979 | CB | PHE | A | 60 | −1.715 | 24.325 | −15.570 | 1.00 | 238.68 |
| 1980 | CG | PHE | A | 60 | −1.824 | 25.601 | −16.362 | 1.00 | 238.68 |
| 1981 | CD1 | PHE | A | 60 | −2.296 | 25.585 | −17.670 | 1.00 | 238.68 |
| 1982 | CD2 | PHE | A | 60 | −1.468 | 26.820 | −15.794 | 1.00 | 238.68 |
| 1983 | CE1 | PHE | A | 60 | −2.411 | 26.758 | −18.400 | 1.00 | 238.68 |
| 1984 | CE2 | PHE | A | 60 | −1.580 | 28.002 | −16.515 | 1.00 | 238.68 |
| 1985 | CZ | PHE | A | 60 | −2.053 | 27.969 | −17.821 | 1.00 | 238.68 |
| 1986 | C | PHE | A | 60 | −0.746 | 23.132 | −13.592 | 1.00 | 178.77 |
| 1987 | O | PHE | A | 60 | −1.468 | 23.162 | −12.588 | 1.00 | 178.77 |
| 1988 | N | GLU | A | 61 | −0.040 | 22.063 | −13.948 | 1.00 | 249.03 |
| 1989 | CA | GLU | A | 61 | −0.076 | 20.822 | −13.181 | 1.00 | 249.03 |
| 1990 | CB | GLU | A | 61 | 0.665 | 19.719 | −13.945 | 1.00 | 249.30 |
| 1991 | CG | GLU | A | 61 | 0.091 | 19.402 | −15.330 | 1.00 | 249.30 |
| 1992 | CD | GLU | A | 61 | 0.076 | 20.605 | −16.264 | 1.00 | 249.30 |
| 1993 | OE1 | GLU | A | 61 | 1.132 | 21.254 | −16.436 | 1.00 | 249.30 |
| 1994 | OE2 | GLU | A | 61 | −0.997 | 20.895 | −16.833 | 1.00 | 249.30 |
| 1995 | C | GLU | A | 61 | 0.537 | 20.991 | −11.792 | 1.00 | 249.03 |
| 1996 | O | GLU | A | 61 | 0.222 | 20.236 | −10.870 | 1.00 | 249.03 |
| 1997 | N | ASP | A | 62 | 1.412 | 21.984 | −11.648 | 1.00 | 157.91 |
| 1998 | CA | ASP | A | 62 | 2.062 | 22.251 | −10.372 | 1.00 | 157.91 |
| 1999 | CB | ASP | A | 62 | 3.191 | 23.264 | −10.539 | 1.00 | 172.18 |
| 2000 | CG | ASP | A | 62 | 4.167 | 22.856 | −11.598 | 1.00 | 172.18 |
| 2001 | OD1 | ASP | A | 62 | 4.368 | 21.633 | −11.779 | 1.00 | 172.18 |
| 2002 | OD2 | ASP | A | 62 | 4.743 | 23.753 | −12.244 | 1.00 | 172.18 |
| 2003 | C | ASP | A | 62 | 1.058 | 22.795 | −9.366 | 1.00 | 157.91 |
| 2004 | O | ASP | A | 62 | 1.266 | 22.700 | −8.159 | 1.00 | 157.91 |
| 2005 | N | SER | A | 63 | −0.026 | 23.384 | −9.864 | 1.00 | 191.12 |
| 2006 | CA | SER | A | 63 | −1.061 | 23.933 | −8.991 | 1.00 | 191.12 |
| 2007 | CB | SER | A | 63 | −2.179 | 24.576 | −9.822 | 1.00 | 203.60 |
| 2008 | OG | SER | A | 63 | −1.685 | 25.593 | −10.671 | 1.00 | 203.60 |
| 2009 | C | SER | A | 63 | −1.634 | 22.778 | −8.186 | 1.00 | 191.12 |
| 2010 | O | SER | A | 63 | −2.040 | 21.773 | −8.753 | 1.00 | 191.12 |
| 2011 | N | GLY | A | 64 | −1.662 | 22.907 | −6.870 | 1.00 | 195.42 |
| 2012 | CA | GLY | A | 64 | −2.199 | 21.821 | −6.087 | 1.00 | 195.42 |
| 2013 | C | GLY | A | 64 | −1.967 | 21.897 | −4.596 | 1.00 | 195.42 |
| 2014 | O | GLY | A | 64 | −1.583 | 22.940 | −4.069 | 1.00 | 195.42 |
| 2015 | N | GLU | A | 65 | −2.199 | 20.765 | −3.933 | 1.00 | 249.69 |
| 2016 | CA | GLU | A | 65 | −2.064 | 20.613 | −2.484 | 1.00 | 249.69 |
| 2017 | CB | GLU | A | 65 | −3.302 | 19.876 | −1.969 | 1.00 | 246.11 |
| 2018 | CG | GLU | A | 65 | −3.277 | 19.481 | −0.514 | 1.00 | 246.11 |
| 2019 | CD | GLU | A | 65 | −4.310 | 18.417 | −0.207 | 1.00 | 246.11 |
| 2020 | OE1 | GLU | A | 65 | −4.201 | 17.309 | −0.779 | 1.00 | 246.11 |
| 2021 | OE2 | GLU | A | 65 | −5.230 | 18.684 | 0.597 | 1.00 | 246.11 |
| 2022 | C | GLU | A | 65 | −0.790 | 19.844 | −2.112 | 1.00 | 249.69 |
| 2023 | O | GLU | A | 65 | −0.613 | 18.711 | −2.540 | 1.00 | 249.69 |
| 2024 | N | TYR | A | 66 | 0.083 | 20.456 | −1.308 | 1.00 | 196.27 |
| 2025 | CA | TYR | A | 66 | 1.334 | 19.818 | −0.890 | 1.00 | 196.27 |
| 2026 | CB | TYR | A | 66 | 2.534 | 20.641 | −1.324 | 1.00 | 181.47 |
| 2027 | CG | TYR | A | 66 | 2.737 | 20.798 | −2.807 | 1.00 | 181.47 |
| 2028 | CD1 | TYR | A | 66 | 1.966 | 21.687 | −3.549 | 1.00 | 181.47 |
| 2029 | CE1 | TYR | A | 66 | 2.227 | 21.911 | −4.896 | 1.00 | 181.47 |
| 2030 | CD2 | TYR | A | 66 | 3.769 | 20.122 | −3.454 | 1.00 | 181.47 |
| 2031 | CE2 | TYR | A | 66 | 4.040 | 20.332 | −4.800 | 1.00 | 181.47 |
| 2032 | CZ | TYR | A | 66 | 3.268 | 21.230 | −5.513 | 1.00 | 181.47 |
| 2033 | OH | TYR | A | 66 | 3.561 | 21.460 | −6.838 | 1.00 | 181.47 |
| 2034 | C | TYR | A | 66 | 1.462 | 19.616 | 0.622 | 1.00 | 196.27 |
| 2035 | O | TYR | A | 66 | 0.665 | 20.149 | 1.402 | 1.00 | 196.27 |
| 2036 | N | LYS | A | 67 | 2.493 | 18.862 | 1.021 | 1.00 | 214.47 |
| 2037 | CA | LYS | A | 67 | 2.778 | 18.572 | 2.435 | 1.00 | 214.47 |
| 2038 | CB | LYS | A | 67 | 1.630 | 17.783 | 3.059 | 1.00 | 179.29 |
| 2039 | CG | LYS | A | 67 | 1.262 | 16.527 | 2.300 | 1.00 | 179.29 |
| 2040 | CD | LYS | A | 67 | 0.071 | 15.859 | 2.955 | 1.00 | 179.29 |
| 2041 | CE | LYS | A | 67 | −0.626 | 14.887 | 2.008 | 1.00 | 179.29 |
| 2042 | NZ | LYS | A | 67 | −1.808 | 14.194 | 2.632 | 1.00 | 179.29 |
| 2043 | C | LYS | A | 67 | 4.077 | 17.799 | 2.681 | 1.00 | 214.47 |
| 2044 | O | LYS | A | 67 | 4.546 | 17.060 | 1.826 | 1.00 | 214.47 |
| 2045 | N | CYS | A | 68 | 4.644 | 17.981 | 3.869 | 1.00 | 202.66 |
| 2046 | CA | CYS | A | 68 | 5.865 | 17.291 | 4.259 | 1.00 | 202.66 |
| 2047 | C | CYS | A | 68 | 5.713 | 16.618 | 5.621 | 1.00 | 202.66 |
| 2048 | O | CYS | A | 68 | 4.961 | 17.075 | 6.483 | 1.00 | 202.66 |
| 2049 | CB | CYS | A | 68 | 7.067 | 18.245 | 4.273 | 1.00 | 195.74 |
| 2050 | SG | CYS | A | 68 | 7.101 | 19.551 | 5.556 | 1.00 | 195.74 |
| 2051 | N | GLN | A | 69 | 6.439 | 15.519 | 5.797 | 1.00 | 233.18 |
| 2052 | CA | GLN | A | 69 | 6.420 | 14.730 | 7.024 | 1.00 | 233.18 |
| 2053 | CB | GLN | A | 69 | 5.367 | 13.631 | 6.896 | 1.00 | 249.69 |
| 2054 | CG | GLN | A | 69 | 5.562 | 12.460 | 7.835 | 1.00 | 249.69 |
| 2055 | CD | GLN | A | 69 | 4.580 | 11.333 | 7.569 | 1.00 | 249.69 |
| 2056 | OE1 | GLN | A | 69 | 4.451 | 10.865 | 6.436 | 1.00 | 249.69 |
| 2057 | NE2 | GLN | A | 69 | 3.888 | 10.885 | 8.615 | 1.00 | 249.69 |
| 2058 | C | GLN | A | 69 | 7.798 | 14.111 | 7.224 | 1.00 | 233.18 |
| 2059 | O | GLN | A | 69 | 8.485 | 13.796 | 6.254 | 1.00 | 233.18 |
| 2060 | N | HIS | A | 70 | 8.206 | 13.942 | 8.477 | 1.00 | 249.54 |
| 2061 | CA | HIS | A | 70 | 9.508 | 13.348 | 8.757 | 1.00 | 249.54 |
| 2062 | CB | HIS | A | 70 | 10.202 | 14.086 | 9.904 | 1.00 | 249.69 |
| 2063 | CG | HIS | A | 70 | 10.674 | 15.458 | 9.536 | 1.00 | 249.69 |
| 2064 | CD2 | HIS | A | 70 | 10.459 | 16.662 | 10.116 | 1.00 | 249.69 |
| 2065 | ND1 | HIS | A | 70 | 11.475 | 15.699 | 8.439 | 1.00 | 249.69 |
| 2066 | CE1 | HIS | A | 70 | 11.731 | 16.992 | 8.359 | 1.00 | 249.69 |
| 2067 | NE2 | HIS | A | 70 | 11.126 | 17.600 | 9.366 | 1.00 | 249.69 |
| 2068 | C | HIS | A | 70 | 9.393 | 11.867 | 9.084 | 1.00 | 249.54 |
| 2069 | O | HIS | A | 70 | 8.327 | 11.270 | 8.917 | 1.00 | 249.54 |
| 2070 | N | GLN | A | 71 | 10.496 | 11.283 | 9.549 | 1.00 | 249.69 |
| 2071 | CA | GLN | A | 71 | 10.546 | 9.863 | 9.894 | 1.00 | 249.69 |
| 2072 | CB | GLN | A | 71 | 11.944 | 9.520 | 10.429 | 1.00 | 249.69 |
| 2073 | CG | GLN | A | 71 | 12.318 | 8.033 | 10.415 | 1.00 | 249.69 |
| 2074 | CD | GLN | A | 71 | 12.356 | 7.432 | 9.015 | 1.00 | 249.69 |
| 2075 | OE1 | GLN | A | 71 | 12.933 | 8.009 | 8.090 | 1.00 | 249.69 |
| 2076 | NE2 | GLN | A | 71 | 11.749 | 6.257 | 8.859 | 1.00 | 249.69 |
| 2077 | C | GLN | A | 71 | 9.474 | 9.485 | 10.925 | 1.00 | 249.69 |
| 2078 | O | GLN | A | 71 | 8.737 | 8.505 | 10.747 | 1.00 | 249.69 |
| 2079 | N | GLN | A | 72 | 9.383 | 10.270 | 11.995 | 1.00 | 249.69 |
| 2080 | CA | GLN | A | 72 | 8.413 | 10.013 | 13.056 | 1.00 | 249.69 |
| 2081 | CB | GLN | A | 72 | 9.148 | 9.484 | 14.292 | 1.00 | 249.69 |
| 2082 | CG | GLN | A | 72 | 8.266 | 9.132 | 15.487 | 1.00 | 249.69 |
| 2083 | CD | GLN | A | 72 | 9.085 | 8.768 | 16.717 | 1.00 | 249.69 |
| 2084 | OE1 | GLN | A | 72 | 9.910 | 7.853 | 16.679 | 1.00 | 249.69 |
| 2085 | NE2 | GLN | A | 72 | 8.860 | 9.486 | 17.817 | 1.00 | 249.69 |
| 2086 | C | GLN | A | 72 | 7.634 | 11.288 | 13.402 | 1.00 | 249.69 |
| 2087 | O | GLN | A | 72 | 7.602 | 11.722 | 14.558 | 1.00 | 249.69 |
| 2088 | N | VAL | A | 73 | 7.011 | 11.891 | 12.393 | 1.00 | 249.69 |
| 2089 | CA | VAL | A | 73 | 6.233 | 13.108 | 12.595 | 1.00 | 249.69 |
| 2090 | CB | VAL | A | 73 | 7.036 | 14.377 | 12.200 | 1.00 | 239.35 |
| 2091 | CG1 | VAL | A | 73 | 6.321 | 15.615 | 12.720 | 1.00 | 239.35 |
| 2092 | CG2 | VAL | A | 73 | 8.449 | 14.304 | 12.750 | 1.00 | 239.35 |
| 2093 | C | VAL | A | 73 | 4.979 | 13.047 | 11.731 | 1.00 | 249.69 |
| 2094 | O | VAL | A | 73 | 5.014 | 12.526 | 10.619 | 1.00 | 249.69 |
| 2095 | N | ASN | A | 74 | 3.875 | 13.578 | 12.245 | 1.00 | 249.69 |
| 2096 | CA | ASN | A | 74 | 2.627 | 13.580 | 11.494 | 1.00 | 249.69 |
| 2097 | CB | ASN | A | 74 | 1.448 | 13.799 | 12.450 | 1.00 | 244.75 |
| 2098 | CG | ASN | A | 74 | 1.421 | 12.775 | 13.581 | 1.00 | 244.75 |
| 2099 | OD1 | ASN | A | 74 | 1.672 | 11.588 | 13.354 | 1.00 | 244.75 |
| 2100 | ND2 | ASN | A | 74 | 1.112 | 13.230 | 14.793 | 1.00 | 244.75 |
| 2101 | C | ASN | A | 74 | 2.667 | 14.663 | 10.404 | 1.00 | 249.69 |
| 2102 | O | ASN | A | 74 | 2.979 | 15.828 | 10.680 | 1.00 | 249.69 |
| 2103 | N | GLU | A | 75 | 2.362 | 14.262 | 9.167 | 1.00 | 249.69 |
| 2104 | CA | GLU | A | 75 | 2.370 | 15.160 | 8.008 | 1.00 | 249.69 |
| 2105 | CB | GLU | A | 75 | 1.656 | 14.485 | 6.826 | 1.00 | 249.69 |
| 2106 | CG | GLU | A | 75 | 0.447 | 13.641 | 7.216 | 1.00 | 249.69 |
| 2107 | CD | GLU | A | 75 | −0.086 | 12.806 | 6.064 | 1.00 | 249.69 |
| 2108 | OE1 | GLU | A | 75 | 0.722 | 12.115 | 5.408 | 1.00 | 249.69 |
| 2109 | OE2 | GLU | A | 75 | −1.312 | 12.831 | 5.820 | 1.00 | 249.69 |
| 2110 | C | GLU | A | 75 | 1.786 | 16.556 | 8.262 | 1.00 | 249.69 |
| 2111 | O | GLU | A | 75 | 0.776 | 16.714 | 8.954 | 1.00 | 249.69 |
| 2112 | N | SER | A | 76 | 2.437 | 17.562 | 7.682 | 1.00 | 249.69 |
| 2113 | CA | SER | A | 76 | 2.037 | 18.959 | 7.833 | 1.00 | 249.69 |
| 2114 | CB | SER | A | 76 | 3.093 | 19.877 | 7.212 | 1.00 | 185.73 |
| 2115 | OG | SER | A | 76 | 3.026 | 19.838 | 5.796 | 1.00 | 185.73 |
| 2116 | C | SER | A | 76 | 0.691 | 19.291 | 7.206 | 1.00 | 249.69 |
| 2117 | O | SER | A | 76 | 0.212 | 18.589 | 6.316 | 1.00 | 249.69 |
| 2118 | N | GLU | A | 77 | 0.093 | 20.382 | 7.677 | 1.00 | 249.69 |
| 2119 | CA | GLU | A | 77 | −1.187 | 20.845 | 7.153 | 1.00 | 249.69 |
| 2120 | CB | GLU | A | 77 | −1.695 | 22.053 | 7.952 | 1.00 | 249.63 |
| 2121 | CG | GLU | A | 77 | −2.038 | 21.734 | 9.394 | 1.00 | 249.63 |
| 2122 | CD | GLU | A | 77 | −3.175 | 20.742 | 9.515 | 1.00 | 249.63 |
| 2123 | OE1 | GLU | A | 77 | −3.606 | 20.194 | 8.477 | 1.00 | 249.63 |
| 2124 | OE2 | GLU | A | 77 | −3.633 | 20.508 | 10.653 | 1.00 | 249.63 |
| 2125 | C | GLU | A | 77 | −0.961 | 21.250 | 5.701 | 1.00 | 249.69 |
| 2126 | O | GLU | A | 77 | −0.262 | 22.226 | 5.423 | 1.00 | 249.69 |
| 2127 | N | PRO | A | 78 | −1.556 | 20.502 | 4.757 | 1.00 | 227.00 |
| 2128 | CD | PRO | A | 78 | −2.599 | 19.491 | 4.999 | 1.00 | 247.88 |
| 2129 | CA | PRO | A | 78 | −1.413 | 20.781 | 3.321 | 1.00 | 227.00 |
| 2130 | CB | PRO | A | 78 | −2.583 | 20.012 | 2.710 | 1.00 | 247.88 |
| 2131 | CG | PRO | A | 78 | −2.752 | 18.854 | 3.641 | 1.00 | 247.88 |
| 2132 | C | PRO | A | 78 | −1.488 | 22.271 | 2.998 | 1.00 | 227.00 |
| 2133 | O | PRO | A | 78 | −2.039 | 23.054 | 3.771 | 1.00 | 227.00 |
| 2134 | N | VAL | A | 79 | −0.910 | 22.665 | 1.871 | 1.00 | 169.93 |
| 2135 | CA | VAL | A | 79 | −0.966 | 24.059 | 1.435 | 1.00 | 169.93 |
| 2136 | CB | VAL | A | 79 | 0.390 | 24.785 | 1.549 | 1.00 | 154.03 |
| 2137 | CG1 | VAL | A | 79 | 0.329 | 26.125 | 0.825 | 1.00 | 154.03 |
| 2138 | CG2 | VAL | A | 79 | 0.721 | 25.022 | 3.014 | 1.00 | 154.03 |
| 2139 | C | VAL | A | 79 | −1.342 | 23.987 | −0.017 | 1.00 | 169.93 |
| 2140 | O | VAL | A | 79 | −0.883 | 23.087 | −0.710 | 1.00 | 169.93 |
| 2141 | N | TYR | A | 80 | −2.175 | 24.911 | −0.487 | 1.00 | 161.75 |
| 2142 | CA | TYR | A | 80 | −2.581 | 24.874 | −1.888 | 1.00 | 161.75 |
| 2143 | CB | TYR | A | 80 | −4.096 | 25.028 | −2.025 | 1.00 | 221.72 |
| 2144 | CG | TYR | A | 80 | −4.606 | 24.573 | −3.372 | 1.00 | 221.72 |
| 2145 | CD1 | TYR | A | 80 | −4.874 | 23.227 | −3.618 | 1.00 | 221.72 |
| 2146 | CE1 | TYR | A | 80 | −5.296 | 22.792 | −4.874 | 1.00 | 221.72 |
| 2147 | CD2 | TYR | A | 80 | −4.773 | 25.478 | −4.418 | 1.00 | 221.72 |
| 2148 | CE2 | TYR | A | 80 | −5.193 | 25.052 | −5.679 | 1.00 | 221.72 |
| 2149 | CZ | TYR | A | 80 | −5.451 | 23.708 | −5.896 | 1.00 | 221.72 |
| 2150 | OH | TYR | A | 80 | −5.860 | 23.276 | −7.134 | 1.00 | 221.72 |
| 2151 | C | TYR | A | 80 | −1.895 | 25.939 | −2.725 | 1.00 | 161.75 |
| 2152 | O | TYR | A | 80 | −1.812 | 27.096 | −2.329 | 1.00 | 161.75 |
| 2153 | N | LEU | A | 81 | −1.405 | 25.534 | −3.889 | 1.00 | 159.92 |
| 2154 | CA | LEU | A | 81 | −0.741 | 26.451 | −4.789 | 1.00 | 159.92 |
| 2155 | CB | LEU | A | 81 | 0.652 | 25.951 | −5.138 | 1.00 | 117.26 |
| 2156 | CG | LEU | A | 81 | 1.353 | 26.823 | −6.188 | 1.00 | 117.26 |
| 2157 | CD1 | LEU | A | 81 | 1.556 | 28.213 | −5.608 | 1.00 | 117.26 |
| 2158 | CD2 | LEU | A | 81 | 2.692 | 26.221 | −6.600 | 1.00 | 117.26 |
| 2159 | C | LEU | A | 81 | −1.550 | 26.562 | −6.067 | 1.00 | 159.92 |
| 2160 | O | LEU | A | 81 | −1.879 | 25.541 | −6.678 | 1.00 | 159.92 |
| 2161 | N | GLU | A | 82 | −1.879 | 27.786 | −6.476 | 1.00 | 176.90 |
| 2162 | CA | GLU | A | 82 | −2.637 | 27.978 | −7.709 | 1.00 | 176.90 |
| 2163 | CB | GLU | A | 82 | −3.950 | 28.697 | −7.427 | 1.00 | 239.33 |
| 2164 | CG | GLU | A | 82 | −5.021 | 28.382 | −8.454 | 1.00 | 239.33 |
| 2165 | CD | GLU | A | 82 | −6.337 | 29.072 | −8.166 | 1.00 | 239.33 |
| 2166 | OE1 | GLU | A | 82 | −6.678 | 29.235 | −6.970 | 1.00 | 239.33 |
| 2167 | OE2 | GLU | A | 82 | −7.038 | 29.437 | −9.139 | 1.00 | 239.33 |
| 2168 | C | GLU | A | 82 | −1.815 | 28.772 | −8.720 | 1.00 | 176.90 |
| 2169 | O | GLU | A | 82 | −1.176 | 29.768 | −8.369 | 1.00 | 176.90 |
| 2170 | N | VAL | A | 83 | −1.818 | 28.315 | −9.973 | 1.00 | 167.08 |
| 2171 | CA | VAL | A | 83 | −1.078 | 28.988 | −11.050 | 1.00 | 167.08 |
| 2172 | CB | VAL | A | 83 | −0.163 | 28.034 | −11.817 | 1.00 | 127.07 |
| 2173 | CG1 | VAL | A | 83 | 0.595 | 28.807 | −12.899 | 1.00 | 127.07 |
| 2174 | CG2 | VAL | A | 83 | 0.800 | 27.368 | −10.847 | 1.00 | 127.07 |
| 2175 | C | VAL | A | 83 | −2.036 | 29.634 | −12.043 | 1.00 | 167.08 |
| 2176 | O | VAL | A | 83 | −3.077 | 29.071 | −12.390 | 1.00 | 167.08 |
| 2177 | N | PHE | A | 84 | −1.653 | 30.810 | −12.524 | 1.00 | 136.71 |
| 2178 | CA | PHE | A | 84 | −2.502 | 31.588 | −13.412 | 1.00 | 136.71 |
| 2179 | CB | PHE | A | 84 | −3.039 | 32.805 | −12.669 | 1.00 | 180.02 |
| 2180 | CG | PHE | A | 84 | −3.878 | 32.481 | −11.481 | 1.00 | 180.02 |
| 2181 | CD1 | PHE | A | 84 | −3.296 | 32.143 | −10.263 | 1.00 | 180.02 |
| 2182 | CD2 | PHE | A | 84 | −5.259 | 32.534 | −11.575 | 1.00 | 180.02 |
| 2183 | CE1 | PHE | A | 84 | −4.085 | 31.871 | −9.157 | 1.00 | 180.02 |
| 2184 | CE2 | PHE | A | 84 | −6.055 | 32.266 | −10.484 | 1.00 | 180.02 |
| 2185 | CZ | PHE | A | 84 | −5.471 | 31.933 | −9.274 | 1.00 | 180.02 |
| 2186 | C | PHE | A | 84 | −1.917 | 32.125 | −14.692 | 1.00 | 136.71 |
| 2187 | O | PHE | A | 84 | −0.710 | 32.289 | −14.838 | 1.00 | 136.71 |
| 2188 | N | SER | A | 85 | −2.822 | 32.440 | −15.606 | 1.00 | 185.25 |
| 2189 | CA | SER | A | 85 | −2.470 | 33.050 | −16.871 | 1.00 | 185.25 |
| 2190 | CB | SER | A | 85 | −2.639 | 32.088 | −18.036 | 1.00 | 191.52 |
| 2191 | OG | SER | A | 85 | −2.269 | 32.736 | −19.246 | 1.00 | 191.52 |
| 2192 | C | SER | A | 85 | −3.462 | 34.193 | −17.012 | 1.00 | 185.25 |
| 2193 | O | SER | A | 85 | −4.680 | 33.960 | −17.105 | 1.00 | 185.25 |
| 2194 | N | ASP | A | 86 | −2.940 | 35.422 | −17.002 | 1.00 | 167.09 |
| 2195 | CA | ASP | A | 86 | −3.769 | 36.623 | −17.117 | 1.00 | 167.09 |
| 2196 | CB | ASP | A | 86 | −4.744 | 36.701 | −15.951 | 1.00 | 156.75 |
| 2197 | CG | ASP | A | 86 | −6.072 | 37.252 | −16.358 | 1.00 | 156.75 |
| 2198 | OD1 | ASP | A | 86 | −6.110 | 38.341 | −16.985 | 1.00 | 156.75 |
| 2199 | OD2 | ASP | A | 86 | −7.083 | 36.587 | −16.045 | 1.00 | 156.75 |
| 2200 | C | ASP | A | 86 | −2.888 | 37.852 | −17.101 | 1.00 | 167.09 |
| 2201 | O | ASP | A | 86 | −1.708 | 37.760 | −16.775 | 1.00 | 167.09 |
| 2202 | N | TRP | A | 87 | −3.455 | 39.005 | −17.438 | 1.00 | 147.13 |
| 2203 | CA | TRP | A | 87 | −2.665 | 40.233 | −17.435 | 1.00 | 147.13 |
| 2204 | CB | TRP | A | 87 | −3.446 | 41.371 | −18.079 | 1.00 | 200.84 |
| 2205 | CG | TRP | A | 87 | −3.221 | 41.441 | −19.553 | 1.00 | 200.84 |
| 2206 | CD2 | TRP | A | 87 | −4.022 | 40.824 | −20.563 | 1.00 | 200.84 |
| 2207 | CE2 | TRP | A | 87 | −3.413 | 41.103 | −21.798 | 1.00 | 200.84 |
| 2208 | CE3 | TRP | A | 87 | −5.199 | 40.061 | −20.542 | 1.00 | 200.84 |
| 2209 | CD1 | TRP | A | 87 | −2.185 | 42.053 | −20.202 | 1.00 | 200.84 |
| 2210 | NE1 | TRP | A | 87 | −2.292 | 41.854 | −21.551 | 1.00 | 200.84 |
| 2211 | CZ2 | TRP | A | 87 | −3.942 | 40.645 | −23.002 | 1.00 | 200.84 |
| 2212 | CZ3 | TRP | A | 87 | −5.726 | 39.602 | −21.752 | 1.00 | 200.84 |
| 2213 | CH2 | TRP | A | 87 | −5.095 | 39.899 | −22.961 | 1.00 | 200.84 |
| 2214 | C | TRP | A | 87 | −2.233 | 40.608 | −16.017 | 1.00 | 147.13 |
| 2215 | O | TRP | A | 87 | −1.040 | 40.785 | −15.743 | 1.00 | 147.13 |
| 2216 | N | LEU | A | 88 | −3.198 | 40.715 | −15.108 | 1.00 | 135.82 |
| 2217 | CA | LEU | A | 88 | −2.886 | 41.049 | −13.725 | 1.00 | 135.82 |
| 2218 | CB | LEU | A | 88 | −3.469 | 42.416 | −13.366 | 1.00 | 139.19 |
| 2219 | CG | LEU | A | 88 | −2.870 | 43.605 | −14.131 | 1.00 | 139.19 |
| 2220 | CD1 | LEU | A | 88 | −3.435 | 44.912 | −13.593 | 1.00 | 139.19 |
| 2221 | CD2 | LEU | A | 88 | −1.360 | 43.608 | −14.008 | 1.00 | 139.19 |
| 2222 | C | LEU | A | 88 | −3.417 | 39.996 | −12.772 | 1.00 | 135.82 |
| 2223 | O | LEU | A | 88 | −4.496 | 39.439 | −12.976 | 1.00 | 135.82 |
| 2224 | N | LEU | A | 89 | −2.644 | 39.710 | −11.736 | 1.00 | 146.34 |
| 2225 | CA | LEU | A | 89 | −3.051 | 38.737 | −10.728 | 1.00 | 146.34 |
| 2226 | CB | LEU | A | 89 | −2.210 | 37.466 | −10.826 | 1.00 | 125.53 |
| 2227 | CG | LEU | A | 89 | −2.519 | 36.431 | −9.741 | 1.00 | 125.53 |
| 2228 | CD1 | LEU | A | 89 | −4.026 | 36.143 | −9.713 | 1.00 | 125.53 |
| 2229 | CD2 | LEU | A | 89 | −1.719 | 35.168 | −10.009 | 1.00 | 125.53 |
| 2230 | C | LEU | A | 89 | −2.854 | 39.355 | −9.354 | 1.00 | 146.34 |
| 2231 | O | LEU | A | 89 | −1.785 | 39.903 | −9.070 | 1.00 | 146.34 |
| 2232 | N | LEU | A | 90 | −3.875 | 39.282 | −8.502 | 1.00 | 124.61 |
| 2233 | CA | LEU | A | 90 | −3.762 | 39.862 | −7.173 | 1.00 | 124.61 |
| 2234 | CB | LEU | A | 90 | −5.132 | 40.294 | −6.687 | 1.00 | 89.03 |
| 2235 | CG | LEU | A | 90 | −5.136 | 40.759 | −5.234 | 1.00 | 89.03 |
| 2236 | CD1 | LEU | A | 90 | −4.192 | 41.932 | −5.091 | 1.00 | 89.03 |
| 2237 | CD2 | LEU | A | 90 | −6.549 | 41.132 | −4.782 | 1.00 | 89.03 |
| 2238 | C | LEU | A | 90 | −3.160 | 38.861 | −6.196 | 1.00 | 124.61 |
| 2239 | O | LEU | A | 90 | −3.766 | 37.842 | −5.902 | 1.00 | 124.61 |
| 2240 | N | GLN | A | 91 | −1.972 | 39.147 | −5.686 | 1.00 | 143.99 |
| 2241 | CA | GLN | A | 91 | −1.335 | 38.229 | −4.757 | 1.00 | 143.99 |
| 2242 | CB | GLN | A | 91 | 0.139 | 38.062 | −5.110 | 1.00 | 163.37 |
| 2243 | CG | GLN | A | 91 | 0.382 | 37.527 | −6.497 | 1.00 | 163.37 |
| 2244 | CD | GLN | A | 91 | 1.861 | 37.383 | −6.798 | 1.00 | 163.37 |
| 2245 | OE1 | GLN | A | 91 | 2.620 | 38.356 | −6.721 | 1.00 | 163.37 |
| 2246 | NE2 | GLN | A | 91 | 2.283 | 36.166 | −7.143 | 1.00 | 163.37 |
| 2247 | C | GLN | A | 91 | −1.463 | 38.676 | −3.304 | 1.00 | 143.99 |
| 2248 | O | GLN | A | 91 | −1.322 | 39.872 | −2.991 | 1.00 | 143.99 |
| 2249 | N | ALA | A | 92 | −1.725 | 37.706 | −2.421 | 1.00 | 122.21 |
| 2250 | CA | ALA | A | 92 | −1.862 | 37.978 | −0.991 | 1.00 | 122.21 |
| 2251 | CB | ALA | A | 92 | −3.283 | 37.702 | −0.548 | 1.00 | 218.43 |
| 2252 | C | ALA | A | 92 | −0.892 | 37.128 | −0.190 | 1.00 | 122.21 |
| 2253 | O | ALA | A | 92 | −0.653 | 35.960 | −0.527 | 1.00 | 122.21 |
| 2254 | N | SER | A | 93 | −0.338 | 37.733 | 0.861 | 1.00 | 143.19 |
| 2255 | CA | SER | A | 93 | 0.612 | 37.067 | 1.742 | 1.00 | 143.19 |
| 2256 | CB | SER | A | 93 | 0.964 | 37.975 | 2.935 | 1.00 | 121.60 |
| 2257 | OG | SER | A | 93 | −0.192 | 38.446 | 3.608 | 1.00 | 121.60 |
| 2258 | C | SER | A | 93 | −0.030 | 35.790 | 2.220 | 1.00 | 143.19 |
| 2259 | O | SER | A | 93 | 0.452 | 34.690 | 1.941 | 1.00 | 143.19 |
| 2260 | N | ALA | A | 94 | −1.121 | 35.958 | 2.948 | 1.00 | 129.43 |
| 2261 | CA | ALA | A | 94 | −1.880 | 34.831 | 3.446 | 1.00 | 129.43 |
| 2262 | CB | ALA | A | 94 | −1.688 | 34.686 | 4.950 | 1.00 | 204.58 |
| 2263 | C | ALA | A | 94 | −3.322 | 35.190 | 3.108 | 1.00 | 129.43 |
| 2264 | O | ALA | A | 94 | −3.620 | 36.387 | 2.969 | 1.00 | 129.43 |
| 2265 | N | GLU | A | 95 | −4.208 | 34.193 | 2.955 | 1.00 | 144.02 |
| 2266 | CA | GLU | A | 95 | −5.601 | 34.499 | 2.632 | 1.00 | 144.02 |
| 2267 | CB | GLU | A | 95 | −6.144 | 33.467 | 1.668 | 1.00 | 173.81 |
| 2268 | CG | GLU | A | 95 | −5.434 | 33.492 | 0.344 | 1.00 | 173.81 |
| 2269 | CD | GLU | A | 95 | −6.123 | 32.642 | −0.695 | 1.00 | 173.81 |
| 2270 | OE1 | GLU | A | 95 | −5.590 | 32.539 | −1.831 | 1.00 | 173.81 |
| 2271 | OE2 | GLU | A | 95 | −7.201 | 32.078 | −0.378 | 1.00 | 173.81 |
| 2272 | C | GLU | A | 95 | −6.488 | 34.587 | 3.879 | 1.00 | 144.02 |
| 2273 | O | GLU | A | 95 | −7.548 | 35.222 | 3.857 | 1.00 | 144.02 |
| 2274 | N | VAL | A | 96 | −6.044 | 33.951 | 4.963 | 1.00 | 165.64 |
| 2275 | CA | VAL | A | 96 | −6.778 | 33.966 | 6.222 | 1.00 | 165.64 |
| 2276 | CB | VAL | A | 96 | −7.256 | 32.573 | 6.592 | 1.00 | 130.98 |
| 2277 | CG1 | VAL | A | 96 | −8.370 | 32.683 | 7.632 | 1.00 | 130.98 |
| 2278 | CG2 | VAL | A | 96 | −7.722 | 31.830 | 5.359 | 1.00 | 130.98 |
| 2279 | C | VAL | A | 96 | −5.864 | 34.458 | 7.335 | 1.00 | 165.64 |
| 2280 | O | VAL | A | 96 | −4.743 | 33.962 | 7.475 | 1.00 | 165.64 |
| 2281 | N | VAL | A | 97 | −6.339 | 35.404 | 8.147 | 1.00 | 117.82 |
| 2282 | CA | VAL | A | 97 | −5.483 | 35.949 | 9.204 | 1.00 | 117.82 |
| 2283 | CB | VAL | A | 97 | −4.908 | 37.299 | 8.784 | 1.00 | 171.13 |
| 2284 | CG1 | VAL | A | 97 | −3.692 | 37.597 | 9.605 | 1.00 | 171.13 |
| 2285 | CG2 | VAL | A | 97 | −4.577 | 37.296 | 7.307 | 1.00 | 171.13 |
| 2286 | C | VAL | A | 97 | −6.078 | 36.149 | 10.585 | 1.00 | 117.82 |
| 2287 | O | VAL | A | 97 | −7.269 | 36.404 | 10.713 | 1.00 | 117.82 |
| 2288 | N | MET | A | 98 | −5.221 | 36.046 | 11.606 | 1.00 | 130.77 |
| 2289 | CA | MET | A | 98 | −5.592 | 36.228 | 13.025 | 1.00 | 130.77 |
| 2290 | CB | MET | A | 98 | −4.587 | 35.504 | 13.927 | 1.00 | 249.69 |
| 2291 | CG | MET | A | 98 | −4.534 | 33.992 | 13.785 | 1.00 | 249.69 |
| 2292 | SD | MET | A | 98 | −5.915 | 33.193 | 14.587 | 1.00 | 249.69 |
| 2293 | CE | MET | A | 98 | −5.438 | 33.351 | 16.321 | 1.00 | 249.69 |
| 2294 | C | MET | A | 98 | −5.545 | 37.712 | 13.360 | 1.00 | 130.77 |
| 2295 | O | MET | A | 98 | −4.509 | 38.332 | 13.187 | 1.00 | 130.77 |
| 2296 | N | GLU | A | 99 | −6.641 | 38.276 | 13.852 | 1.00 | 130.20 |
| 2297 | CA | GLU | A | 99 | −6.679 | 39.701 | 14.167 | 1.00 | 130.20 |
| 2298 | CB | GLU | A | 99 | −7.773 | 39.989 | 15.194 | 1.00 | 216.65 |
| 2299 | CG | GLU | A | 99 | −8.283 | 41.423 | 15.162 | 1.00 | 216.65 |
| 2300 | CD | GLU | A | 99 | −9.161 | 41.757 | 16.355 | 1.00 | 216.65 |
| 2301 | OE1 | GLU | A | 99 | −9.931 | 40.874 | 16.795 | 1.00 | 216.65 |
| 2302 | OE2 | GLU | A | 99 | −9.088 | 42.904 | 16.844 | 1.00 | 216.65 |
| 2303 | C | GLU | A | 99 | −5.341 | 40.155 | 14.729 | 1.00 | 130.20 |
| 2304 | O | GLU | A | 99 | −4.832 | 39.538 | 15.672 | 1.00 | 130.20 |
| 2305 | N | GLY | A | 100 | −4.752 | 41.206 | 14.157 | 1.00 | 150.30 |
| 2306 | CA | GLY | A | 100 | −3.476 | 41.685 | 14.674 | 1.00 | 150.30 |
| 2307 | C | GLY | A | 100 | −2.232 | 41.342 | 13.869 | 1.00 | 150.30 |
| 2308 | O | GLY | A | 100 | −1.210 | 42.016 | 13.999 | 1.00 | 150.30 |
| 2309 | N | GLN | A | 101 | −2.305 | 40.305 | 13.043 | 1.00 | 149.89 |
| 2310 | CA | GLN | A | 101 | −1.173 | 39.891 | 12.209 | 1.00 | 149.89 |
| 2311 | CB | GLN | A | 101 | −1.385 | 38.471 | 11.699 | 1.00 | 220.06 |
| 2312 | CG | GLN | A | 101 | −1.255 | 37.429 | 12.777 | 1.00 | 220.06 |
| 2313 | CD | GLN | A | 101 | −0.056 | 37.694 | 13.650 | 1.00 | 220.06 |
| 2314 | OE1 | GLN | A | 101 | −0.048 | 38.646 | 14.431 | 1.00 | 220.06 |
| 2315 | NE2 | GLN | A | 101 | 0.974 | 36.862 | 13.516 | 1.00 | 220.06 |
| 2316 | C | GLN | A | 101 | −0.918 | 40.831 | 11.033 | 1.00 | 149.89 |
| 2317 | O | GLN | A | 101 | −1.663 | 41.773 | 10.795 | 1.00 | 149.89 |
| 2318 | N | PRO | A | 102 | 0.161 | 40.572 | 10.277 | 1.00 | 131.84 |
| 2319 | CD | PRO | A | 102 | 1.289 | 39.692 | 10.571 | 1.00 | 202.39 |
| 2320 | CA | PRO | A | 102 | 0.440 | 41.436 | 9.131 | 1.00 | 131.84 |
| 2321 | CB | PRO | A | 102 | 1.966 | 41.376 | 9.067 | 1.00 | 202.39 |
| 2322 | CG | PRO | A | 102 | 2.222 | 39.941 | 9.395 | 1.00 | 202.39 |
| 2323 | C | PRO | A | 102 | −0.216 | 40.960 | 7.837 | 1.00 | 131.84 |
| 2324 | O | PRO | A | 102 | −0.418 | 39.749 | 7.627 | 1.00 | 131.84 |
| 2325 | N | LEU | A | 103 | −0.531 | 41.915 | 6.960 | 1.00 | 120.38 |
| 2326 | CA | LEU | A | 103 | −1.150 | 41.589 | 5.695 | 1.00 | 120.38 |
| 2327 | CB | LEU | A | 103 | −2.620 | 41.909 | 5.782 | 1.00 | 119.50 |
| 2328 | CG | LEU | A | 103 | −3.321 | 41.329 | 4.569 | 1.00 | 119.50 |
| 2329 | CD1 | LEU | A | 103 | −3.261 | 39.821 | 4.686 | 1.00 | 119.50 |
| 2330 | CD2 | LEU | A | 103 | −4.753 | 41.816 | 4.475 | 1.00 | 119.50 |
| 2331 | C | LEU | A | 103 | −0.542 | 42.345 | 4.508 | 1.00 | 120.38 |
| 2332 | O | LEU | A | 103 | −0.471 | 43.582 | 4.537 | 1.00 | 120.38 |
| 2333 | N | PHE | A | 104 | −0.116 | 41.629 | 3.462 | 1.00 | 130.73 |
| 2334 | CA | PHE | A | 104 | 0.451 | 42.300 | 2.288 | 1.00 | 130.73 |
| 2335 | CB | PHE | A | 104 | 1.943 | 42.002 | 2.144 | 1.00 | 196.69 |
| 2336 | CG | PHE | A | 104 | 2.747 | 42.332 | 3.353 | 1.00 | 196.69 |
| 2337 | CD1 | PHE | A | 104 | 2.753 | 41.480 | 4.446 | 1.00 | 196.69 |
| 2338 | CD2 | PHE | A | 104 | 3.493 | 43.499 | 3.410 | 1.00 | 196.69 |
| 2339 | CE1 | PHE | A | 104 | 3.495 | 41.787 | 5.589 | 1.00 | 196.69 |
| 2340 | CE2 | PHE | A | 104 | 4.237 | 43.815 | 4.545 | 1.00 | 196.69 |
| 2341 | CZ | PHE | A | 104 | 4.238 | 42.960 | 5.638 | 1.00 | 196.69 |
| 2342 | C | PHE | A | 104 | −0.236 | 41.868 | 1.004 | 1.00 | 130.73 |
| 2343 | O | PHE | A | 104 | −0.279 | 40.673 | 0.695 | 1.00 | 130.73 |
| 2344 | N | LEU | A | 105 | −0.774 | 42.830 | 0.258 | 1.00 | 119.49 |
| 2345 | CA | LEU | A | 105 | −1.421 | 42.514 | −1.015 | 1.00 | 119.49 |
| 2346 | CB | LEU | A | 105 | −2.813 | 43.107 | −1.066 | 1.00 | 119.62 |
| 2347 | CG | LEU | A | 105 | −3.735 | 42.614 | 0.049 | 1.00 | 119.62 |
| 2348 | CD1 | LEU | A | 105 | −5.138 | 43.152 | −0.172 | 1.00 | 119.62 |
| 2349 | CD2 | LEU | A | 105 | −3.738 | 41.092 | 0.063 | 1.00 | 119.62 |
| 2350 | C | LEU | A | 105 | −0.579 | 43.084 | −2.137 | 1.00 | 119.49 |
| 2351 | O | LEU | A | 105 | 0.091 | 44.104 | −1.971 | 1.00 | 119.49 |
| 2352 | N | ARG | A | 106 | −0.614 | 42.446 | −3.291 | 1.00 | 119.67 |
| 2353 | CA | ARG | A | 106 | 0.216 | 42.926 | −4.373 | 1.00 | 119.67 |
| 2354 | CB | ARG | A | 106 | 1.510 | 42.109 | −4.377 | 1.00 | 158.51 |
| 2355 | CG | ARG | A | 106 | 2.513 | 42.493 | −5.412 | 1.00 | 158.51 |
| 2356 | CD | ARG | A | 106 | 3.656 | 41.503 | −5.450 | 1.00 | 158.51 |
| 2357 | NE | ARG | A | 106 | 4.522 | 41.787 | −6.581 | 1.00 | 158.51 |
| 2358 | CZ | ARG | A | 106 | 5.231 | 40.876 | −7.233 | 1.00 | 158.51 |
| 2359 | NH1 | ARG | A | 106 | 5.180 | 39.604 | −6.860 | 1.00 | 158.51 |
| 2360 | NH2 | ARG | A | 106 | 5.973 | 41.239 | −8.280 | 1.00 | 158.51 |
| 2361 | C | ARG | A | 106 | −0.505 | 42.808 | −5.717 | 1.00 | 119.67 |
| 2362 | O | ARG | A | 106 | −1.026 | 41.737 | −6.069 | 1.00 | 119.67 |
| 2363 | N | CYS | A | 107 | −0.564 | 43.915 | −6.455 | 1.00 | 120.58 |
| 2364 | CA | CYS | A | 107 | −1.189 | 43.904 | −7.774 | 1.00 | 120.58 |
| 2365 | C | CYS | A | 107 | −0.053 | 43.480 | −8.666 | 1.00 | 120.58 |
| 2366 | O | CYS | A | 107 | 0.836 | 44.299 | −8.953 | 1.00 | 120.58 |
| 2367 | CB | CYS | A | 107 | −1.645 | 45.304 | −8.168 | 1.00 | 140.98 |
| 2368 | SG | CYS | A | 107 | −2.754 | 45.384 | −9.622 | 1.00 | 140.98 |
| 2369 | N | HIS | A | 108 | −0.069 | 42.210 | −9.083 | 1.00 | 148.29 |
| 2370 | CA | HIS | A | 108 | 1.002 | 41.644 | −9.914 | 1.00 | 148.29 |
| 2371 | CB | HIS | A | 108 | 1.309 | 40.222 | −9.470 | 1.00 | 171.13 |
| 2372 | CG | HIS | A | 108 | 2.556 | 39.660 | −10.068 | 1.00 | 171.13 |
| 2373 | CD2 | HIS | A | 108 | 2.793 | 38.483 | −10.691 | 1.00 | 171.13 |
| 2374 | ND1 | HIS | A | 108 | 3.773 | 40.308 | −10.001 | 1.00 | 171.13 |
| 2375 | CE1 | HIS | A | 108 | 4.702 | 39.549 | −10.548 | 1.00 | 171.13 |
| 2376 | NE2 | HIS | A | 108 | 4.136 | 38.434 | −10.975 | 1.00 | 171.13 |
| 2377 | C | HIS | A | 108 | 0.759 | 41.632 | −11.411 | 1.00 | 148.29 |
| 2378 | O | HIS | A | 108 | −0.248 | 41.082 | −11.896 | 1.00 | 148.29 |
| 2379 | N | GLY | A | 109 | 1.701 | 42.229 | −12.136 | 1.00 | 189.63 |
| 2380 | CA | GLY | A | 109 | 1.593 | 42.286 | −13.579 | 1.00 | 189.63 |
| 2381 | C | GLY | A | 109 | 2.109 | 41.002 | −14.172 | 1.00 | 189.63 |
| 2382 | O | GLY | A | 109 | 2.735 | 40.217 | −13.469 | 1.00 | 189.63 |
| 2383 | N | TRP | A | 110 | 1.836 | 40.782 | −15.454 | 1.00 | 151.88 |
| 2384 | CA | TRP | A | 110 | 2.302 | 39.582 | −16.136 | 1.00 | 151.88 |
| 2385 | CB | TRP | A | 110 | 1.381 | 39.246 | −17.307 | 1.00 | 208.61 |
| 2386 | CG | TRP | A | 110 | 1.896 | 38.147 | −18.184 | 1.00 | 208.61 |
| 2387 | CD2 | TRP | A | 110 | 1.495 | 36.764 | −18.170 | 1.00 | 208.61 |
| 2388 | CE2 | TRP | A | 110 | 2.277 | 36.100 | −19.135 | 1.00 | 208.61 |
| 2389 | CE3 | TRP | A | 110 | 0.556 | 36.022 | −17.434 | 1.00 | 208.61 |
| 2390 | CD1 | TRP | A | 110 | 2.863 | 38.255 | −19.134 | 1.00 | 208.61 |
| 2391 | NE1 | TRP | A | 110 | 3.103 | 37.034 | −19.708 | 1.00 | 208.61 |
| 2392 | CZ2 | TRP | A | 110 | 2.147 | 34.723 | −19.387 | 1.00 | 208.61 |
| 2393 | CZ3 | TRP | A | 110 | 0.429 | 34.653 | −17.688 | 1.00 | 208.61 |
| 2394 | CH2 | TRP | A | 110 | 1.218 | 34.024 | −18.658 | 1.00 | 208.61 |
| 2395 | C | TRP | A | 110 | 3.747 | 39.773 | −16.615 | 1.00 | 151.88 |
| 2396 | O | TRP | A | 110 | 4.182 | 40.909 | −16.869 | 1.00 | 151.88 |
| 2397 | N | ARG | A | 111 | 4.490 | 38.666 | −16.714 | 1.00 | 149.24 |
| 2398 | CA | ARG | A | 111 | 5.892 | 38.712 | −17.125 | 1.00 | 149.24 |
| 2399 | CB | ARG | A | 111 | 6.013 | 39.012 | −18.619 | 1.00 | 249.69 |
| 2400 | CG | ARG | A | 111 | 6.011 | 37.777 | −19.494 | 1.00 | 249.69 |
| 2401 | CD | ARG | A | 111 | 6.475 | 38.106 | −20.902 | 1.00 | 249.69 |
| 2402 | NE | ARG | A | 111 | 7.340 | 37.059 | −21.427 | 1.00 | 249.69 |
| 2403 | CZ | ARG | A | 111 | 8.490 | 36.693 | −20.868 | 1.00 | 249.69 |
| 2404 | NH1 | ARG | A | 111 | 8.916 | 37.285 | −19.757 | 1.00 | 249.69 |
| 2405 | NH2 | ARG | A | 111 | 9.215 | 35.721 | −21.411 | 1.00 | 249.69 |
| 2406 | C | ARG | A | 111 | 6.655 | 39.777 | −16.336 | 1.00 | 149.24 |
| 2407 | O | ARG | A | 111 | 7.605 | 40.391 | −16.825 | 1.00 | 149.24 |
| 2408 | N | ASN | A | 112 | 6.219 | 39.988 | −15.107 | 1.00 | 174.92 |
| 2409 | CA | ASN | A | 112 | 6.837 | 40.958 | −14.231 | 1.00 | 174.92 |
| 2410 | CB | ASN | A | 112 | 8.189 | 40.434 | −13.735 | 1.00 | 206.21 |
| 2411 | CG | ASN | A | 112 | 8.698 | 41.184 | −12.514 | 1.00 | 206.21 |
| 2412 | OD1 | ASN | A | 112 | 8.201 | 42.256 | −12.171 | 1.00 | 206.21 |
| 2413 | ND2 | ASN | A | 112 | 9.703 | 40.620 | −11.858 | 1.00 | 206.21 |
| 2414 | C | ASN | A | 112 | 7.029 | 42.293 | −14.937 | 1.00 | 174.92 |
| 2415 | O | ASN | A | 112 | 7.992 | 43.001 | −14.647 | 1.00 | 174.92 |
| 2416 | N | TRP | A | 113 | 6.129 | 42.643 | −15.861 | 1.00 | 198.50 |
| 2417 | CA | TRP | A | 113 | 6.233 | 43.932 | −16.547 | 1.00 | 198.50 |
| 2418 | CB | TRP | A | 113 | 5.232 | 44.051 | −17.676 | 1.00 | 235.99 |
| 2419 | CG | TRP | A | 113 | 5.669 | 43.414 | −18.929 | 1.00 | 235.99 |
| 2420 | CD2 | TRP | A | 113 | 4.825 | 42.775 | −19.896 | 1.00 | 235.99 |
| 2421 | CE2 | TRP | A | 113 | 5.653 | 42.372 | −20.965 | 1.00 | 235.99 |
| 2422 | CE3 | TRP | A | 113 | 3.455 | 42.498 | −19.949 | 1.00 | 235.99 |
| 2423 | CD1 | TRP | A | 113 | 6.927 | 43.381 | −19.440 | 1.00 | 235.99 |
| 2424 | NE1 | TRP | A | 113 | 6.931 | 42.753 | −20.665 | 1.00 | 235.99 |
| 2425 | CZ2 | TRP | A | 113 | 5.150 | 41.705 | −22.088 | 1.00 | 235.99 |
| 2426 | CZ3 | TRP | A | 113 | 2.952 | 41.837 | −21.067 | 1.00 | 235.99 |
| 2427 | CH2 | TRP | A | 113 | 3.801 | 41.452 | −22.124 | 1.00 | 235.99 |
| 2428 | C | TRP | A | 113 | 5.948 | 45.050 | −15.563 | 1.00 | 198.50 |
| 2429 | O | TRP | A | 113 | 5.891 | 44.821 | −14.356 | 1.00 | 198.50 |
| 2430 | N | ASP | A | 114 | 5.765 | 46.262 | −16.069 | 1.00 | 220.71 |
| 2431 | CA | ASP | A | 114 | 5.476 | 47.388 | −15.188 | 1.00 | 220.71 |
| 2432 | CB | ASP | A | 114 | 6.471 | 48.542 | −15.432 | 1.00 | 249.69 |
| 2433 | CG | ASP | A | 114 | 7.802 | 48.353 | −14.692 | 1.00 | 249.69 |
| 2434 | OD1 | ASP | A | 114 | 7.791 | 48.237 | −13.446 | 1.00 | 249.69 |
| 2435 | OD2 | ASP | A | 114 | 8.863 | 48.329 | −15.356 | 1.00 | 249.69 |
| 2436 | C | ASP | A | 114 | 4.037 | 47.881 | −15.366 | 1.00 | 220.71 |
| 2437 | O | ASP | A | 114 | 3.569 | 48.065 | −16.501 | 1.00 | 220.71 |
| 2438 | N | VAL | A | 115 | 3.337 | 48.072 | −14.242 | 1.00 | 122.27 |
| 2439 | CA | VAL | A | 115 | 1.960 | 48.556 | −14.268 | 1.00 | 122.27 |
| 2440 | CB | VAL | A | 115 | 1.032 | 47.648 | −13.440 | 1.00 | 142.42 |
| 2441 | CG1 | VAL | A | 115 | −0.418 | 47.957 | −13.777 | 1.00 | 142.42 |
| 2442 | CG2 | VAL | A | 115 | 1.328 | 46.195 | −13.716 | 1.00 | 142.42 |
| 2443 | C | VAL | A | 115 | 1.889 | 49.978 | −13.705 | 1.00 | 122.27 |
| 2444 | O | VAL | A | 115 | 2.566 | 50.311 | −12.726 | 1.00 | 122.27 |
| 2445 | N | TYR | A | 116 | 1.061 | 50.800 | −14.336 | 1.00 | 125.74 |
| 2446 | CA | TYR | A | 116 | 0.885 | 52.181 | −13.923 | 1.00 | 125.74 |
| 2447 | CB | TYR | A | 116 | 1.328 | 53.115 | −15.058 | 1.00 | 233.81 |
| 2448 | CG | TYR | A | 116 | 2.797 | 53.014 | −15.357 | 1.00 | 233.81 |
| 2449 | CD1 | TYR | A | 116 | 3.272 | 52.218 | −16.397 | 1.00 | 233.81 |
| 2450 | CE1 | TYR | A | 116 | 4.644 | 52.092 | −16.639 | 1.00 | 233.81 |
| 2451 | CD2 | TYR | A | 116 | 3.720 | 53.683 | −14.568 | 1.00 | 233.81 |
| 2452 | CE2 | TYR | A | 116 | 5.089 | 53.566 | −14.796 | 1.00 | 233.81 |
| 2453 | CZ | TYR | A | 116 | 5.551 | 52.773 | −15.832 | 1.00 | 233.81 |
| 2454 | OH | TYR | A | 116 | 6.914 | 52.664 | −16.054 | 1.00 | 233.81 |
| 2455 | C | TYR | A | 116 | −0.578 | 52.470 | −13.539 | 1.00 | 125.74 |
| 2456 | O | TYR | A | 116 | −1.451 | 51.626 | −13.747 | 1.00 | 125.74 |
| 2457 | N | LYS | A | 117 | −0.833 | 53.665 | −12.992 | 1.00 | 145.27 |
| 2458 | CA | LYS | A | 117 | −2.176 | 54.095 | −12.586 | 1.00 | 145.27 |
| 2459 | CB | LYS | A | 117 | −3.020 | 54.515 | −13.801 | 1.00 | 191.01 |
| 2460 | CG | LYS | A | 117 | −2.807 | 55.955 | −14.269 | 1.00 | 191.01 |
| 2461 | CD | LYS | A | 117 | −3.969 | 56.437 | −15.140 | 1.00 | 191.01 |
| 2462 | CE | LYS | A | 117 | −5.304 | 56.363 | −14.377 | 1.00 | 191.01 |
| 2463 | NZ | LYS | A | 117 | −6.504 | 56.811 | −15.161 | 1.00 | 191.01 |
| 2464 | C | LYS | A | 117 | −2.913 | 53.014 | −11.814 | 1.00 | 145.27 |
| 2465 | O | LYS | A | 117 | −4.053 | 52.653 | −12.141 | 1.00 | 145.27 |
| 2466 | N | VAL | A | 118 | −2.269 | 52.518 | −10.767 | 1.00 | 149.03 |
| 2467 | CA | VAL | A | 118 | −2.858 | 51.469 | −9.959 | 1.00 | 149.03 |
| 2468 | CB | VAL | A | 118 | −1.761 | 50.602 | −9.356 | 1.00 | 99.24 |
| 2469 | CG1 | VAL | A | 118 | −2.233 | 49.921 | −8.081 | 1.00 | 99.24 |
| 2470 | CG2 | VAL | A | 118 | −1.364 | 49.558 | −10.374 | 1.00 | 99.24 |
| 2471 | C | VAL | A | 118 | −3.816 | 51.912 | −8.858 | 1.00 | 149.03 |
| 2472 | O | VAL | A | 118 | −3.601 | 52.931 | −8.179 | 1.00 | 149.03 |
| 2473 | N | ILE | A | 119 | −4.878 | 51.115 | −8.706 | 1.00 | 111.26 |
| 2474 | CA | ILE | A | 119 | −5.923 | 51.325 | −7.714 | 1.00 | 111.26 |
| 2475 | CB | ILE | A | 119 | −7.157 | 51.950 | −8.351 | 1.00 | 110.34 |
| 2476 | CG2 | ILE | A | 119 | −8.183 | 52.293 | −7.286 | 1.00 | 110.34 |
| 2477 | CG1 | ILE | A | 119 | −6.752 | 53.187 | −9.108 | 1.00 | 110.34 |
| 2478 | CD1 | ILE | A | 119 | −7.726 | 53.537 | −10.166 | 1.00 | 110.34 |
| 2479 | C | ILE | A | 119 | −6.349 | 49.972 | −7.162 | 1.00 | 111.26 |
| 2480 | O | ILE | A | 119 | −6.641 | 49.054 | −7.919 | 1.00 | 111.26 |
| 2481 | N | TYR | A | 120 | −6.378 | 49.843 | −5.848 | 1.00 | 126.83 |
| 2482 | CA | TYR | A | 120 | −6.829 | 48.603 | −5.266 | 1.00 | 126.83 |
| 2483 | CB | TYR | A | 120 | −6.039 | 48.270 | −4.015 | 1.00 | 126.10 |
| 2484 | CG | TYR | A | 120 | −4.615 | 47.904 | −4.280 | 1.00 | 126.10 |
| 2485 | CD1 | TYR | A | 120 | −3.636 | 48.884 | −4.399 | 1.00 | 126.10 |
| 2486 | CE1 | TYR | A | 120 | −2.315 | 48.542 | −4.657 | 1.00 | 126.10 |
| 2487 | CD2 | TYR | A | 120 | −4.243 | 46.569 | −4.427 | 1.00 | 126.10 |
| 2488 | CE2 | TYR | A | 120 | −2.930 | 46.213 | −4.687 | 1.00 | 126.10 |
| 2489 | CZ | TYR | A | 120 | −1.968 | 47.197 | −4.802 | 1.00 | 126.10 |
| 2490 | OH | TYR | A | 120 | −0.662 | 46.817 | −5.067 | 1.00 | 126.10 |
| 2491 | C | TYR | A | 120 | −8.280 | 48.818 | −4.889 | 1.00 | 126.83 |
| 2492 | O | TYR | A | 120 | −8.672 | 49.956 | −4.606 | 1.00 | 126.83 |
| 2493 | N | TYR | A | 121 | −9.084 | 47.753 | −4.890 | 1.00 | 106.78 |
| 2494 | CA | TYR | A | 121 | −10.496 | 47.884 | −4.516 | 1.00 | 106.78 |
| 2495 | CB | TYR | A | 121 | −11.417 | 47.731 | −5.734 | 1.00 | 155.39 |
| 2496 | CG | TYR | A | 121 | −11.362 | 48.833 | −6.777 | 1.00 | 155.39 |
| 2497 | CD1 | TYR | A | 121 | −10.181 | 49.122 | −7.459 | 1.00 | 155.39 |
| 2498 | CE1 | TYR | A | 121 | −10.149 | 50.069 | −8.499 | 1.00 | 155.39 |
| 2499 | CD2 | TYR | A | 121 | −12.516 | 49.522 | −7.151 | 1.00 | 155.39 |
| 2500 | CE2 | TYR | A | 121 | −12.496 | 50.467 | −8.187 | 1.00 | 155.39 |
| 2501 | CZ | TYR | A | 121 | −11.309 | 50.732 | −8.858 | 1.00 | 155.39 |
| 2502 | OH | TYR | A | 121 | −11.272 | 51.633 | −9.903 | 1.00 | 155.39 |
| 2503 | C | TYR | A | 121 | −10.892 | 46.838 | −3.482 | 1.00 | 106.78 |
| 2504 | O | TYR | A | 121 | −10.544 | 45.657 | −3.611 | 1.00 | 106.78 |
| 2505 | N | LYS | A | 122 | −11.618 | 47.278 | −2.457 | 1.00 | 141.82 |
| 2506 | CA | LYS | A | 122 | −12.108 | 46.362 | −1.440 | 1.00 | 141.82 |
| 2507 | CB | LYS | A | 122 | −11.511 | 46.674 | −0.080 | 1.00 | 249.31 |
| 2508 | CG | LYS | A | 122 | −11.983 | 45.716 | 0.997 | 1.00 | 249.31 |
| 2509 | CD | LYS | A | 122 | −11.631 | 46.233 | 2.367 | 1.00 | 249.31 |
| 2510 | CE | LYS | A | 122 | −12.252 | 45.380 | 3.451 | 1.00 | 249.31 |
| 2511 | NZ | LYS | A | 122 | −12.022 | 46.005 | 4.773 | 1.00 | 249.31 |
| 2512 | C | LYS | A | 122 | −13.631 | 46.511 | −1.375 | 1.00 | 141.82 |
| 2513 | O | LYS | A | 122 | −14.136 | 47.587 | −1.042 | 1.00 | 141.82 |
| 2514 | N | ASP | A | 123 | −14.349 | 45.433 | −1.703 | 1.00 | 127.31 |
| 2515 | CA | ASP | A | 123 | −15.801 | 45.436 | −1.701 | 1.00 | 127.31 |
| 2516 | CB | ASP | A | 123 | −16.344 | 45.574 | −0.276 | 1.00 | 199.26 |
| 2517 | CG | ASP | A | 123 | −16.186 | 44.302 | 0.531 | 1.00 | 199.26 |
| 2518 | OD1 | ASP | A | 123 | −16.571 | 43.223 | 0.031 | 1.00 | 199.26 |
| 2519 | OD2 | ASP | A | 123 | −15.685 | 44.378 | 1.668 | 1.00 | 199.26 |
| 2520 | C | ASP | A | 123 | −16.395 | 46.531 | −2.584 | 1.00 | 127.31 |
| 2521 | O | ASP | A | 123 | −17.238 | 47.311 | −2.132 | 1.00 | 127.31 |
| 2522 | N | GLY | A | 124 | −15.955 | 46.579 | −3.842 | 1.00 | 152.83 |
| 2523 | CA | GLY | A | 124 | −16.468 | 47.561 | −4.788 | 1.00 | 152.83 |
| 2524 | C | GLY | A | 124 | −16.067 | 49.007 | −4.590 | 1.00 | 152.83 |
| 2525 | O | GLY | A | 124 | −16.394 | 49.847 | −5.425 | 1.00 | 152.83 |
| 2526 | N | GLU | A | 125 | −15.355 | 49.297 | −3.502 | 1.00 | 121.95 |
| 2527 | CA | GLU | A | 125 | −14.912 | 50.668 | −3.185 | 1.00 | 121.95 |
| 2528 | CB | GLU | A | 125 | −15.037 | 50.926 | −1.673 | 1.00 | 249.20 |
| 2529 | CG | GLU | A | 125 | −16.464 | 51.006 | −1.143 | 1.00 | 249.20 |
| 2530 | CD | GLU | A | 125 | −17.154 | 52.304 | −1.519 | 1.00 | 249.20 |
| 2531 | OE1 | GLU | A | 125 | −16.680 | 53.373 | −1.081 | 1.00 | 249.20 |
| 2532 | OE2 | GLU | A | 125 | −18.168 | 52.253 | −2.250 | 1.00 | 249.20 |
| 2533 | C | GLU | A | 125 | −13.479 | 50.956 | −3.612 | 1.00 | 121.95 |
| 2534 | O | GLU | A | 125 | −12.616 | 50.071 | −3.548 | 1.00 | 121.95 |
| 2535 | N | ALA | A | 126 | −13.236 | 52.185 | −4.059 | 1.00 | 117.59 |
| 2536 | CA | ALA | A | 126 | −11.886 | 52.572 | −4.444 | 1.00 | 117.59 |
| 2537 | CB | ALA | A | 126 | −11.912 | 53.935 | −5.116 | 1.00 | 242.07 |
| 2538 | C | ALA | A | 126 | −11.102 | 52.638 | −3.129 | 1.00 | 117.59 |
| 2539 | O | ALA | A | 126 | −11.619 | 53.123 | −2.132 | 1.00 | 117.59 |
| 2540 | N | LEU | A | 127 | −9.862 | 52.176 | −3.112 | 1.00 | 119.50 |
| 2541 | CA | LEU | A | 127 | −9.140 | 52.177 | −1.857 | 1.00 | 119.50 |
| 2542 | CB | LEU | A | 127 | −8.845 | 50.751 | −1.411 | 1.00 | 117.16 |
| 2543 | CG | LEU | A | 127 | −8.750 | 50.671 | 0.099 | 1.00 | 117.16 |
| 2544 | CD1 | LEU | A | 127 | −10.045 | 51.245 | 0.705 | 1.00 | 117.16 |
| 2545 | CD2 | LEU | A | 127 | −8.534 | 49.234 | 0.528 | 1.00 | 117.16 |
| 2546 | C | LEU | A | 127 | −7.859 | 52.960 | −1.813 | 1.00 | 119.50 |
| 2547 | O | LEU | A | 127 | −7.738 | 53.890 | −1.030 | 1.00 | 119.50 |
| 2548 | N | LYS | A | 128 | −6.880 | 52.566 | −2.616 | 1.00 | 140.05 |
| 2549 | CA | LYS | A | 128 | −5.603 | 53.269 | −2.661 | 1.00 | 140.05 |
| 2550 | CB | LYS | A | 128 | −4.503 | 52.411 | −2.036 | 1.00 | 182.59 |
| 2551 | CG | LYS | A | 128 | −4.725 | 52.060 | −0.576 | 1.00 | 182.59 |
| 2552 | CD | LYS | A | 128 | −4.526 | 53.260 | 0.337 | 1.00 | 182.59 |
| 2553 | CE | LYS | A | 128 | −4.657 | 52.855 | 1.804 | 1.00 | 182.59 |
| 2554 | NZ | LYS | A | 128 | −4.240 | 53.938 | 2.743 | 1.00 | 182.59 |
| 2555 | C | LYS | A | 128 | −5.293 | 53.534 | −4.127 | 1.00 | 140.05 |
| 2556 | O | LYS | A | 128 | −5.875 | 52.899 | −5.010 | 1.00 | 140.05 |
| 2557 | N | TYR | A | 129 | −4.386 | 54.467 | −4.393 | 1.00 | 141.11 |
| 2558 | CA | TYR | A | 129 | −4.021 | 54.780 | −5.779 | 1.00 | 141.11 |
| 2559 | CB | TYR | A | 129 | −4.977 | 55.812 | −6.344 | 1.00 | 146.88 |
| 2560 | CG | TYR | A | 129 | −4.437 | 56.491 | −7.574 | 1.00 | 146.88 |
| 2561 | CD1 | TYR | A | 129 | −4.560 | 55.909 | −8.824 | 1.00 | 146.88 |
| 2562 | CE1 | TYR | A | 129 | −4.010 | 56.518 | −9.957 | 1.00 | 146.88 |
| 2563 | CD2 | TYR | A | 129 | −3.749 | 57.705 | −7.474 | 1.00 | 146.88 |
| 2564 | CE2 | TYR | A | 129 | −3.191 | 58.313 | −8.593 | 1.00 | 146.88 |
| 2565 | CZ | TYR | A | 129 | −3.327 | 57.717 | −9.835 | 1.00 | 146.88 |
| 2566 | OH | TYR | A | 129 | −2.781 | 58.324 | −10.946 | 1.00 | 146.88 |
| 2567 | C | TYR | A | 129 | −2.588 | 55.294 | −5.941 | 1.00 | 141.11 |
| 2568 | O | TYR | A | 129 | −2.098 | 56.070 | −5.107 | 1.00 | 141.11 |
| 2569 | N | TRP | A | 130 | −1.919 | 54.861 | −7.014 | 1.00 | 137.60 |
| 2570 | CA | TRP | A | 130 | −0.545 | 55.292 | −7.282 | 1.00 | 137.60 |
| 2571 | CB | TRP | A | 130 | 0.487 | 54.377 | −6.621 | 1.00 | 197.40 |
| 2572 | CG | TRP | A | 130 | 0.244 | 54.027 | −5.190 | 1.00 | 197.40 |
| 2573 | CD2 | TRP | A | 130 | 0.940 | 54.526 | −4.060 | 1.00 | 197.40 |
| 2574 | CE2 | TRP | A | 130 | 0.426 | 53.878 | −2.911 | 1.00 | 197.40 |
| 2575 | CE3 | TRP | A | 130 | 1.974 | 55.458 | −3.892 | 1.00 | 197.40 |
| 2576 | CD1 | TRP | A | 130 | −0.661 | 53.124 | −4.702 | 1.00 | 197.40 |
| 2577 | NE1 | TRP | A | 130 | −0.561 | 53.018 | −3.335 | 1.00 | 197.40 |
| 2578 | CZ2 | TRP | A | 130 | 0.893 | 54.128 | −1.624 | 1.00 | 197.40 |
| 2579 | CZ3 | TRP | A | 130 | 2.452 | 55.713 | −2.598 | 1.00 | 197.40 |
| 2580 | CH2 | TRP | A | 130 | 1.906 | 55.055 | −1.484 | 1.00 | 197.40 |
| 2581 | C | TRP | A | 130 | −0.262 | 55.287 | −8.780 | 1.00 | 137.60 |
| 2582 | O | TRP | A | 130 | −1.055 | 54.764 | −9.576 | 1.00 | 137.60 |
| 2583 | N | TYR | A | 131 | 0.879 | 55.868 | −9.156 | 1.00 | 159.17 |
| 2584 | CA | TYR | A | 131 | 1.313 | 55.920 | −10.554 | 1.00 | 159.17 |
| 2585 | CB | TYR | A | 131 | 2.164 | 57.155 | −10.788 | 1.00 | 169.09 |
| 2586 | CG | TYR | A | 131 | 2.407 | 57.426 | −12.241 | 1.00 | 169.09 |
| 2587 | CD1 | TYR | A | 131 | 1.394 | 57.927 | −13.051 | 1.00 | 169.09 |
| 2588 | CE1 | TYR | A | 131 | 1.603 | 58.144 | −14.415 | 1.00 | 169.09 |
| 2589 | CD2 | TYR | A | 131 | 3.642 | 57.150 | −12.821 | 1.00 | 169.09 |
| 2590 | CE2 | TYR | A | 131 | 3.863 | 57.363 | −14.183 | 1.00 | 169.09 |
| 2591 | CZ | TYR | A | 131 | 2.842 | 57.858 | −14.974 | 1.00 | 169.09 |
| 2592 | OH | TYR | A | 131 | 3.073 | 58.051 | −16.318 | 1.00 | 169.09 |
| 2593 | C | TYR | A | 131 | 2.152 | 54.655 | −10.756 | 1.00 | 159.17 |
| 2594 | O | TYR | A | 131 | 1.619 | 53.613 | −11.140 | 1.00 | 159.17 |
| 2595 | N | GLU | A | 132 | 3.464 | 54.754 | −10.524 | 1.00 | 172.97 |
| 2596 | CA | GLU | A | 132 | 4.328 | 53.577 | −10.594 | 1.00 | 172.97 |
| 2597 | CB | GLU | A | 132 | 5.777 | 53.933 | −10.237 | 1.00 | 249.69 |
| 2598 | CG | GLU | A | 132 | 6.593 | 54.598 | −11.341 | 1.00 | 249.69 |
| 2599 | CD | GLU | A | 132 | 7.784 | 53.750 | −11.767 | 1.00 | 249.69 |
| 2600 | OE1 | GLU | A | 132 | 8.097 | 52.767 | −11.058 | 1.00 | 249.69 |
| 2601 | OE2 | GLU | A | 132 | 8.411 | 54.063 | −12.804 | 1.00 | 249.69 |
| 2602 | C | GLU | A | 132 | 3.677 | 52.838 | −9.436 | 1.00 | 172.97 |
| 2603 | O | GLU | A | 132 | 3.531 | 53.413 | −8.343 | 1.00 | 172.97 |
| 2604 | N | ASN | A | 133 | 3.285 | 51.580 | −9.628 | 1.00 | 204.08 |
| 2605 | CA | ASN | A | 133 | 2.559 | 50.925 | −8.545 | 1.00 | 204.08 |
| 2606 | CB | ASN | A | 133 | 1.839 | 49.637 | −9.065 | 1.00 | 217.94 |
| 2607 | CG | ASN | A | 133 | 2.705 | 48.389 | −9.085 | 1.00 | 217.94 |
| 2608 | OD1 | ASN | A | 133 | 3.862 | 48.418 | −9.493 | 1.00 | 217.94 |
| 2609 | ND2 | ASN | A | 133 | 2.114 | 47.262 | −8.676 | 1.00 | 217.94 |
| 2610 | C | ASN | A | 133 | 3.229 | 50.722 | −7.190 | 1.00 | 204.08 |
| 2611 | O | ASN | A | 133 | 4.322 | 51.214 | −6.922 | 1.00 | 204.08 |
| 2612 | N | HIS | A | 134 | 2.512 | 50.050 | −6.314 | 1.00 | 188.50 |
| 2613 | CA | HIS | A | 134 | 2.986 | 49.818 | −4.979 | 1.00 | 188.50 |
| 2614 | CB | HIS | A | 134 | 2.434 | 50.910 | −4.067 | 1.00 | 249.69 |
| 2615 | CG | HIS | A | 134 | 3.005 | 50.881 | −2.679 | 1.00 | 249.69 |
| 2616 | CD2 | HIS | A | 134 | 2.405 | 50.671 | −1.481 | 1.00 | 249.69 |
| 2617 | ND1 | HIS | A | 134 | 4.338 | 51.055 | −2.429 | 1.00 | 249.69 |
| 2618 | CE1 | HIS | A | 134 | 4.557 | 50.953 | −1.120 | 1.00 | 249.69 |
| 2619 | NE2 | HIS | A | 134 | 3.399 | 50.720 | −0.530 | 1.00 | 249.69 |
| 2620 | C | HIS | A | 134 | 2.485 | 48.456 | −4.542 | 1.00 | 188.50 |
| 2621 | O | HIS | A | 134 | 2.068 | 47.636 | −5.374 | 1.00 | 188.50 |
| 2622 | N | ASN | A | 135 | 2.518 | 48.217 | −3.234 | 1.00 | 122.11 |
| 2623 | CA | ASN | A | 135 | 2.076 | 46.946 | −2.670 | 1.00 | 122.11 |
| 2624 | CB | ASN | A | 135 | 3.274 | 45.986 | −2.547 | 1.00 | 249.69 |
| 2625 | CG | ASN | A | 135 | 3.803 | 45.530 | −3.906 | 1.00 | 249.69 |
| 2626 | OD1 | ASN | A | 135 | 3.010 | 45.158 | −4.776 | 1.00 | 249.69 |
| 2627 | ND2 | ASN | A | 135 | 5.128 | 45.534 | −4.086 | 1.00 | 249.69 |
| 2628 | C | ASN | A | 135 | 1.431 | 47.190 | −1.301 | 1.00 | 122.11 |
| 2629 | O | ASN | A | 135 | 2.081 | 47.042 | −0.271 | 1.00 | 122.11 |
| 2630 | N | ILE | A | 136 | 0.151 | 47.572 | −1.313 | 1.00 | 110.87 |
| 2631 | CA | ILE | A | 136 | −0.653 | 47.846 | −0.115 | 1.00 | 110.87 |
| 2632 | CB | ILE | A | 136 | −2.147 | 47.756 | −0.452 | 1.00 | 153.19 |
| 2633 | CG2 | ILE | A | 136 | −2.474 | 46.406 | −1.055 | 1.00 | 153.19 |
| 2634 | CG1 | ILE | A | 136 | −2.973 | 47.969 | 0.801 | 1.00 | 153.19 |
| 2635 | CD1 | ILE | A | 136 | −4.463 | 47.811 | 0.553 | 1.00 | 153.19 |
| 2636 | C | ILE | A | 136 | −0.350 | 46.912 | 1.075 | 1.00 | 110.87 |
| 2637 | O | ILE | A | 136 | −0.773 | 45.732 | 1.121 | 1.00 | 110.87 |
| 2638 | N | SER | A | 137 | 0.351 | 47.475 | 2.061 | 1.00 | 139.22 |
| 2639 | CA | SER | A | 137 | 0.763 | 46.734 | 3.254 | 1.00 | 139.22 |
| 2640 | CB | SER | A | 137 | 2.242 | 46.951 | 3.488 | 1.00 | 151.42 |
| 2641 | OG | SER | A | 137 | 2.597 | 46.432 | 4.746 | 1.00 | 151.42 |
| 2642 | C | SER | A | 137 | 0.032 | 47.027 | 4.554 | 1.00 | 139.22 |
| 2643 | O | SER | A | 137 | −0.378 | 48.149 | 4.823 | 1.00 | 139.22 |
| 2644 | N | ILE | A | 138 | −0.080 | 45.991 | 5.374 | 1.00 | 158.60 |
| 2645 | CA | ILE | A | 138 | −0.760 | 46.072 | 6.659 | 1.00 | 158.60 |
| 2646 | CB | ILE | A | 138 | −2.136 | 45.423 | 6.577 | 1.00 | 139.81 |
| 2647 | CG2 | ILE | A | 138 | −2.695 | 45.183 | 7.958 | 1.00 | 139.81 |
| 2648 | CG1 | ILE | A | 138 | −3.058 | 46.314 | 5.766 | 1.00 | 139.81 |
| 2649 | CD1 | ILE | A | 138 | −4.349 | 45.652 | 5.392 | 1.00 | 139.81 |
| 2650 | C | ILE | A | 138 | 0.014 | 45.396 | 7.780 | 1.00 | 158.60 |
| 2651 | O | ILE | A | 138 | 0.321 | 44.203 | 7.716 | 1.00 | 158.60 |
| 2652 | N | THR | A | 139 | 0.309 | 46.174 | 8.816 | 1.00 | 172.47 |
| 2653 | CA | THR | A | 139 | 1.053 | 45.695 | 9.975 | 1.00 | 172.47 |
| 2654 | CB | THR | A | 139 | 1.698 | 46.877 | 10.697 | 1.00 | 249.69 |
| 2655 | OG1 | THR | A | 139 | 0.689 | 47.853 | 10.990 | 1.00 | 249.69 |
| 2656 | CG2 | THR | A | 139 | 2.763 | 47.522 | 9.811 | 1.00 | 249.69 |
| 2657 | C | THR | A | 139 | 0.107 | 44.974 | 10.920 | 1.00 | 172.47 |
| 2658 | O | THR | A | 139 | 0.027 | 43.744 | 10.930 | 1.00 | 172.47 |
| 2659 | N | ASN | A | 140 | −0.601 | 45.760 | 11.719 | 1.00 | 189.96 |
| 2660 | CA | ASN | A | 140 | −1.573 | 45.233 | 12.662 | 1.00 | 189.96 |
| 2661 | CB | ASN | A | 140 | −1.738 | 46.209 | 13.830 | 1.00 | 189.00 |
| 2662 | CG | ASN | A | 140 | −2.767 | 45.750 | 14.835 | 1.00 | 189.00 |
| 2663 | OD1 | ASN | A | 140 | −3.887 | 45.403 | 14.466 | 1.00 | 189.00 |
| 2664 | ND2 | ASN | A | 140 | −2.398 | 45.764 | 16.111 | 1.00 | 189.00 |
| 2665 | C | ASN | A | 140 | −2.877 | 45.113 | 11.879 | 1.00 | 189.96 |
| 2666 | O | ASN | A | 140 | −3.367 | 46.110 | 11.335 | 1.00 | 189.96 |
| 2667 | N | ALA | A | 141 | −3.437 | 43.904 | 11.818 | 1.00 | 161.77 |
| 2668 | CA | ALA | A | 141 | −4.669 | 43.674 | 11.062 | 1.00 | 161.77 |
| 2669 | CB | ALA | A | 141 | −4.569 | 42.364 | 10.287 | 1.00 | 147.99 |
| 2670 | C | ALA | A | 141 | −5.962 | 43.694 | 11.874 | 1.00 | 161.77 |
| 2671 | O | ALA | A | 141 | −6.105 | 42.992 | 12.888 | 1.00 | 161.77 |
| 2672 | N | THR | A | 142 | −6.902 | 44.510 | 11.402 | 1.00 | 148.72 |
| 2673 | CA | THR | A | 142 | −8.208 | 44.652 | 12.032 | 1.00 | 148.72 |
| 2674 | CB | THR | A | 142 | −8.792 | 46.049 | 11.793 | 1.00 | 197.74 |
| 2675 | OG1 | THR | A | 142 | −7.820 | 47.046 | 12.140 | 1.00 | 197.74 |
| 2676 | CG2 | THR | A | 142 | −10.036 | 46.245 | 12.636 | 1.00 | 197.74 |
| 2677 | C | THR | A | 142 | −9.126 | 43.636 | 11.376 | 1.00 | 148.72 |
| 2678 | O | THR | A | 142 | −8.849 | 43.153 | 10.277 | 1.00 | 148.72 |
| 2679 | N | VAL | A | 143 | −10.224 | 43.310 | 12.037 | 1.00 | 167.20 |
| 2680 | CA | VAL | A | 143 | −11.143 | 42.347 | 11.456 | 1.00 | 167.20 |
| 2681 | CB | VAL | A | 143 | −12.142 | 41.824 | 12.479 | 1.00 | 139.08 |
| 2682 | CG1 | VAL | A | 143 | −13.146 | 42.910 | 12.826 | 1.00 | 139.08 |
| 2683 | CG2 | VAL | A | 143 | −12.838 | 40.590 | 11.934 | 1.00 | 139.08 |
| 2684 | C | VAL | A | 143 | −11.930 | 43.001 | 10.334 | 1.00 | 167.20 |
| 2685 | O | VAL | A | 143 | −12.432 | 42.315 | 9.446 | 1.00 | 167.20 |
| 2686 | N | GLU | A | 144 | −12.047 | 44.327 | 10.372 | 1.00 | 193.27 |
| 2687 | CA | GLU | A | 144 | −12.785 | 45.043 | 9.333 | 1.00 | 193.27 |
| 2688 | CB | GLU | A | 144 | −13.048 | 46.488 | 9.742 | 1.00 | 249.50 |
| 2689 | CG | GLU | A | 144 | −13.868 | 46.624 | 11.002 | 1.00 | 249.50 |
| 2690 | CD | GLU | A | 144 | −13.060 | 47.202 | 12.141 | 1.00 | 249.50 |
| 2691 | OE1 | GLU | A | 144 | −12.590 | 48.352 | 12.003 | 1.00 | 249.50 |
| 2692 | OE2 | GLU | A | 144 | −12.888 | 46.511 | 13.170 | 1.00 | 249.50 |
| 2693 | C | GLU | A | 144 | −12.022 | 45.019 | 8.019 | 1.00 | 193.27 |
| 2694 | O | GLU | A | 144 | −12.572 | 45.370 | 6.981 | 1.00 | 193.27 |
| 2695 | N | ASP | A | 145 | −10.755 | 44.606 | 8.069 | 1.00 | 179.52 |
| 2696 | CA | ASP | A | 145 | −9.931 | 44.524 | 6.866 | 1.00 | 179.52 |
| 2697 | CB | ASP | A | 145 | −8.449 | 44.383 | 7.228 | 1.00 | 162.18 |
| 2698 | CG | ASP | A | 145 | −7.836 | 45.691 | 7.667 | 1.00 | 162.18 |
| 2699 | OD1 | ASP | A | 145 | −7.903 | 46.662 | 6.883 | 1.00 | 162.18 |
| 2700 | OD2 | ASP | A | 145 | −7.286 | 45.750 | 8.786 | 1.00 | 162.18 |
| 2701 | C | ASP | A | 145 | −10.357 | 43.348 | 6.000 | 1.00 | 179.52 |
| 2702 | O | ASP | A | 145 | −10.003 | 43.286 | 4.819 | 1.00 | 179.52 |
| 2703 | N | SER | A | 146 | −11.117 | 42.421 | 6.592 | 1.00 | 145.41 |
| 2704 | CA | SER | A | 146 | −11.600 | 41.223 | 5.886 | 1.00 | 145.41 |
| 2705 | CB | SER | A | 146 | −12.318 | 40.267 | 6.856 | 1.00 | 152.87 |
| 2706 | OG | SER | A | 146 | −11.477 | 39.816 | 7.907 | 1.00 | 152.87 |
| 2707 | C | SER | A | 146 | −12.565 | 41.619 | 4.782 | 1.00 | 145.41 |
| 2708 | O | SER | A | 146 | −13.518 | 42.349 | 5.037 | 1.00 | 145.41 |
| 2709 | N | GLY | A | 147 | −12.323 | 41.140 | 3.563 | 1.00 | 168.10 |
| 2710 | CA | GLY | A | 147 | −13.215 | 41.477 | 2.467 | 1.00 | 168.10 |
| 2711 | C | GLY | A | 147 | −12.794 | 40.871 | 1.149 | 1.00 | 168.10 |
| 2712 | O | GLY | A | 147 | −12.011 | 39.907 | 1.129 | 1.00 | 168.10 |
| 2713 | N | THR | A | 148 | −13.306 | 41.428 | 0.048 | 1.00 | 117.05 |
| 2714 | CA | THR | A | 148 | −12.961 | 40.922 | −1.283 | 1.00 | 117.05 |
| 2715 | CB | THR | A | 148 | −14.234 | 40.419 | −2.014 | 1.00 | 146.29 |
| 2716 | OG1 | THR | A | 148 | −14.738 | 41.446 | −2.867 | 1.00 | 146.29 |
| 2717 | CG2 | THR | A | 148 | −15.327 | 40.059 | −1.012 | 1.00 | 146.29 |
| 2718 | C | THR | A | 148 | −12.235 | 42.004 | −2.116 | 1.00 | 117.05 |
| 2719 | O | THR | A | 148 | −12.833 | 43.019 | −2.502 | 1.00 | 117.05 |
| 2720 | N | TYR | A | 149 | −10.948 | 41.776 | −2.386 | 1.00 | 131.53 |
| 2721 | CA | TYR | A | 149 | −10.136 | 42.735 | −3.128 | 1.00 | 131.53 |
| 2722 | CB | TYR | A | 149 | −8.772 | 42.906 | −2.453 | 1.00 | 104.84 |
| 2723 | CG | TYR | A | 149 | −8.803 | 43.338 | −1.003 | 1.00 | 104.84 |
| 2724 | CD1 | TYR | A | 149 | −9.126 | 42.435 | 0.010 | 1.00 | 104.84 |
| 2725 | CE1 | TYR | A | 149 | −9.127 | 42.823 | 1.361 | 1.00 | 104.84 |
| 2726 | CD2 | TYR | A | 149 | −8.477 | 44.643 | −0.639 | 1.00 | 104.84 |
| 2727 | CE2 | TYR | A | 149 | −8.467 | 45.042 | 0.700 | 1.00 | 104.84 |
| 2728 | CZ | TYR | A | 149 | −8.792 | 44.133 | 1.696 | 1.00 | 104.84 |
| 2729 | OH | TYR | A | 149 | −8.778 | 44.535 | 3.019 | 1.00 | 104.84 |
| 2730 | C | TYR | A | 149 | −9.881 | 42.371 | −4.589 | 1.00 | 131.53 |
| 2731 | O | TYR | A | 149 | −10.064 | 41.216 | −4.989 | 1.00 | 131.53 |
| 2732 | N | TYR | A | 150 | −9.454 | 43.380 | −5.362 | 1.00 | 105.68 |
| 2733 | CA | TYR | A | 150 | −9.090 | 43.272 | −6.784 | 1.00 | 105.68 |
| 2734 | CB | TYR | A | 150 | −10.319 | 43.023 | −7.660 | 1.00 | 139.51 |
| 2735 | CG | TYR | A | 150 | −11.173 | 44.231 | −7.964 | 1.00 | 139.51 |
| 2736 | CD1 | TYR | A | 150 | −10.738 | 45.210 | −8.856 | 1.00 | 139.51 |
| 2737 | CE1 | TYR | A | 150 | −11.568 | 46.306 | −9.205 | 1.00 | 139.51 |
| 2738 | CD2 | TYR | A | 150 | −12.456 | 44.369 | −7.413 | 1.00 | 139.51 |
| 2739 | CE2 | TYR | A | 150 | −13.294 | 45.456 | −7.759 | 1.00 | 139.51 |
| 2740 | CZ | TYR | A | 150 | −12.839 | 46.418 | −8.660 | 1.00 | 139.51 |
| 2741 | OH | TYR | A | 150 | −13.648 | 47.469 | −9.041 | 1.00 | 139.51 |
| 2742 | C | TYR | A | 150 | −8.429 | 44.587 | −7.146 | 1.00 | 105.68 |
| 2743 | O | TYR | A | 150 | −8.720 | 45.604 | −6.525 | 1.00 | 105.68 |
| 2744 | N | CYS | A | 151 | −7.536 | 44.585 | −8.128 | 1.00 | 99.57 |
| 2745 | CA | CYS | A | 151 | −6.868 | 45.830 | −8.510 | 1.00 | 99.57 |
| 2746 | C | CYS | A | 151 | −6.994 | 46.133 | −9.992 | 1.00 | 99.57 |
| 2747 | O | CYS | A | 151 | −7.274 | 45.244 | −10.786 | 1.00 | 99.57 |
| 2748 | CB | CYS | A | 151 | −5.393 | 45.773 | −8.136 | 1.00 | 148.54 |
| 2749 | SG | CYS | A | 151 | −4.448 | 44.452 | −8.958 | 1.00 | 148.54 |
| 2750 | N | THR | A | 152 | −6.782 | 47.396 | −10.359 | 1.00 | 107.45 |
| 2751 | CA | THR | A | 152 | −6.857 | 47.838 | −11.756 | 1.00 | 107.45 |
| 2752 | CB | THR | A | 152 | −8.058 | 48.753 | −11.986 | 1.00 | 135.55 |
| 2753 | OG1 | THR | A | 152 | −7.802 | 50.045 | −11.407 | 1.00 | 135.55 |
| 2754 | CG2 | THR | A | 152 | −9.295 | 48.160 | −11.345 | 1.00 | 135.55 |
| 2755 | C | THR | A | 152 | −5.600 | 48.637 | −12.118 | 1.00 | 107.45 |
| 2756 | O | THR | A | 152 | −5.043 | 49.358 | −11.285 | 1.00 | 107.45 |
| 2757 | N | GLY | A | 153 | −5.159 | 48.518 | −13.359 | 1.00 | 123.72 |
| 2758 | CA | GLY | A | 153 | −3.971 | 49.241 | −13.744 | 1.00 | 123.72 |
| 2759 | C | GLY | A | 153 | −3.749 | 49.248 | −15.239 | 1.00 | 123.72 |
| 2760 | O | GLY | A | 153 | −4.388 | 48.477 | −15.965 | 1.00 | 123.72 |
| 2761 | N | LYS | A | 154 | −2.834 | 50.113 | −15.691 | 1.00 | 129.98 |
| 2762 | CA | LYS | A | 154 | −2.515 | 50.256 | −17.107 | 1.00 | 129.98 |
| 2763 | CB | LYS | A | 154 | −2.490 | 51.740 | −17.481 | 1.00 | 212.29 |
| 2764 | CG | LYS | A | 154 | −2.291 | 52.012 | −18.945 | 1.00 | 212.29 |
| 2765 | CD | LYS | A | 154 | −2.351 | 53.502 | −19.225 | 1.00 | 212.29 |
| 2766 | CE | LYS | A | 154 | −2.074 | 53.803 | −20.693 | 1.00 | 212.29 |
| 2767 | NZ | LYS | A | 154 | −2.101 | 55.266 | −20.986 | 1.00 | 212.29 |
| 2768 | C | LYS | A | 154 | −1.179 | 49.591 | −17.445 | 1.00 | 129.98 |
| 2769 | O | LYS | A | 154 | −0.130 | 49.969 | −16.923 | 1.00 | 129.98 |
| 2770 | N | VAL | A | 155 | −1.242 | 48.579 | −18.311 | 1.00 | 168.08 |
| 2771 | CA | VAL | A | 155 | −0.071 | 47.827 | −18.769 | 1.00 | 168.08 |
| 2772 | CB | VAL | A | 155 | −0.355 | 46.305 | −18.759 | 1.00 | 187.90 |
| 2773 | CG1 | VAL | A | 155 | 0.825 | 45.540 | −19.304 | 1.00 | 187.90 |
| 2774 | CG2 | VAL | A | 155 | −0.658 | 45.849 | −17.343 | 1.00 | 187.90 |
| 2775 | C | VAL | A | 155 | 0.193 | 48.277 | −20.196 | 1.00 | 168.08 |
| 2776 | O | VAL | A | 155 | −0.717 | 48.244 | −21.025 | 1.00 | 168.08 |
| 2777 | N | TRP | A | 156 | 1.429 | 48.672 | −20.493 | 1.00 | 179.87 |
| 2778 | CA | TRP | A | 156 | 1.765 | 49.167 | −21.834 | 1.00 | 179.87 |
| 2779 | CB | TRP | A | 156 | 1.399 | 48.169 | −22.954 | 1.00 | 249.69 |
| 2780 | CG | TRP | A | 156 | 2.200 | 46.890 | −23.019 | 1.00 | 249.69 |
| 2781 | CD2 | TRP | A | 156 | 3.591 | 46.752 | −23.359 | 1.00 | 249.69 |
| 2782 | CE2 | TRP | A | 156 | 3.900 | 45.367 | −23.302 | 1.00 | 249.69 |
| 2783 | CE3 | TRP | A | 156 | 4.606 | 47.657 | −23.714 | 1.00 | 249.69 |
| 2784 | CD1 | TRP | A | 156 | 1.740 | 45.625 | −22.774 | 1.00 | 249.69 |
| 2785 | NE1 | TRP | A | 156 | 2.754 | 44.707 | −22.945 | 1.00 | 249.69 |
| 2786 | CZ2 | TRP | A | 156 | 5.175 | 44.871 | −23.581 | 1.00 | 249.69 |
| 2787 | CZ3 | TRP | A | 156 | 5.876 | 47.161 | −23.993 | 1.00 | 249.69 |
| 2788 | CH2 | TRP | A | 156 | 6.148 | 45.779 | −23.921 | 1.00 | 249.69 |
| 2789 | C | TRP | A | 156 | 0.905 | 50.400 | −22.020 | 1.00 | 179.87 |
| 2790 | O | TRP | A | 156 | 1.242 | 51.485 | −21.535 | 1.00 | 179.87 |
| 2791 | N | GLN | A | 157 | −0.221 | 50.212 | −22.712 | 1.00 | 176.07 |
| 2792 | CA | GLN | A | 157 | −1.161 | 51.301 | −22.959 | 1.00 | 176.07 |
| 2793 | CB | GLN | A | 157 | −0.864 | 51.955 | −24.313 | 1.00 | 249.69 |
| 2794 | CG | GLN | A | 157 | 0.381 | 52.851 | −24.311 | 1.00 | 249.69 |
| 2795 | CD | GLN | A | 157 | 0.232 | 54.078 | −23.412 | 1.00 | 249.69 |
| 2796 | OE1 | GLN | A | 157 | −0.638 | 54.922 | −23.635 | 1.00 | 249.69 |
| 2797 | NE2 | GLN | A | 157 | 1.086 | 54.180 | −22.391 | 1.00 | 249.69 |
| 2798 | C | GLN | A | 157 | −2.643 | 50.904 | −22.872 | 1.00 | 176.07 |
| 2799 | O | GLN | A | 157 | −3.504 | 51.584 | −23.430 | 1.00 | 176.07 |
| 2800 | N | LEU | A | 158 | −2.937 | 49.811 | −22.171 | 1.00 | 165.71 |
| 2801 | CA | LEU | A | 158 | −4.320 | 49.375 | −21.989 | 1.00 | 165.71 |
| 2802 | CB | LEU | A | 158 | −4.611 | 48.122 | −22.809 | 1.00 | 232.32 |
| 2803 | CG | LEU | A | 158 | −4.809 | 48.278 | −24.317 | 1.00 | 232.32 |
| 2804 | CD1 | LEU | A | 158 | −5.868 | 47.274 | −24.764 | 1.00 | 232.32 |
| 2805 | CD2 | LEU | A | 158 | −5.267 | 49.687 | −24.663 | 1.00 | 232.32 |
| 2806 | C | LEU | A | 158 | −4.642 | 49.105 | −20.520 | 1.00 | 165.71 |
| 2807 | O | LEU | A | 158 | −3.764 | 48.726 | −19.741 | 1.00 | 165.71 |
| 2808 | N | ASP | A | 159 | −5.903 | 49.301 | −20.148 | 1.00 | 172.26 |
| 2809 | CA | ASP | A | 159 | −6.321 | 49.080 | −18.772 | 1.00 | 172.26 |
| 2810 | CB | ASP | A | 159 | −7.518 | 49.974 | −18.431 | 1.00 | 159.32 |
| 2811 | CG | ASP | A | 159 | −7.259 | 51.435 | −18.725 | 1.00 | 159.32 |
| 2812 | OD1 | ASP | A | 159 | −6.345 | 52.022 | −18.103 | 1.00 | 159.32 |
| 2813 | OD2 | ASP | A | 159 | −7.974 | 51.995 | −19.586 | 1.00 | 159.32 |
| 2814 | C | ASP | A | 159 | −6.702 | 47.618 | −18.556 | 1.00 | 172.26 |
| 2815 | O | ASP | A | 159 | −7.192 | 46.960 | −19.479 | 1.00 | 172.26 |
| 2816 | N | TYR | A | 160 | −6.468 | 47.113 | −17.343 | 1.00 | 165.86 |
| 2817 | CA | TYR | A | 160 | −6.826 | 45.741 | −17.016 | 1.00 | 165.86 |
| 2818 | CB | TYR | A | 160 | −5.667 | 44.810 | −17.253 | 1.00 | 170.32 |
| 2819 | CG | TYR | A | 160 | −5.121 | 44.887 | −18.643 | 1.00 | 170.32 |
| 2820 | CD1 | TYR | A | 160 | −4.101 | 45.789 | −18.959 | 1.00 | 170.32 |
| 2821 | CE1 | TYR | A | 160 | −3.558 | 45.844 | −20.241 | 1.00 | 170.32 |
| 2822 | CD2 | TYR | A | 160 | −5.600 | 44.042 | −19.645 | 1.00 | 170.32 |
| 2823 | CE2 | TYR | A | 160 | −5.072 | 44.088 | −20.937 | 1.00 | 170.32 |
| 2824 | CZ | TYR | A | 160 | −4.046 | 44.988 | −21.228 | 1.00 | 170.32 |
| 2825 | OH | TYR | A | 160 | −3.493 | 45.018 | −22.495 | 1.00 | 170.32 |
| 2826 | C | TYR | A | 160 | −7.301 | 45.590 | −15.586 | 1.00 | 165.86 |
| 2827 | O | TYR | A | 160 | −6.845 | 46.289 | −14.675 | 1.00 | 165.86 |
| 2828 | N | GLU | A | 161 | −8.228 | 44.660 | −15.410 | 1.00 | 140.66 |
| 2829 | CA | GLU | A | 161 | −8.828 | 44.380 | −14.119 | 1.00 | 140.66 |
| 2830 | CB | GLU | A | 161 | −10.356 | 44.448 | −14.272 | 1.00 | 201.20 |
| 2831 | CG | GLU | A | 161 | −11.197 | 44.125 | −13.044 | 1.00 | 201.20 |
| 2832 | CD | GLU | A | 161 | −12.653 | 44.551 | −13.222 | 1.00 | 201.20 |
| 2833 | OE1 | GLU | A | 161 | −13.525 | 44.011 | −12.499 | 1.00 | 201.20 |
| 2834 | OE2 | GLU | A | 161 | −12.920 | 45.434 | −14.076 | 1.00 | 201.20 |
| 2835 | C | GLU | A | 161 | −8.339 | 42.987 | −13.688 | 1.00 | 140.66 |
| 2836 | O | GLU | A | 161 | −8.148 | 42.095 | −14.523 | 1.00 | 140.66 |
| 2837 | N | SER | A | 162 | −8.109 | 42.815 | −12.389 | 1.00 | 133.72 |
| 2838 | CA | SER | A | 162 | −7.628 | 41.552 | −11.841 | 1.00 | 133.72 |
| 2839 | CB | SER | A | 162 | −6.687 | 41.835 | −10.672 | 1.00 | 153.53 |
| 2840 | OG | SER | A | 162 | −7.366 | 42.515 | −9.616 | 1.00 | 153.53 |
| 2841 | C | SER | A | 162 | −8.766 | 40.686 | −11.341 | 1.00 | 133.72 |
| 2842 | O | SER | A | 162 | −9.852 | 41.187 | −11.066 | 1.00 | 133.72 |
| 2843 | N | GLU | A | 163 | −8.517 | 39.386 | −11.222 | 1.00 | 190.14 |
| 2844 | CA | GLU | A | 163 | −9.539 | 38.488 | −10.699 | 1.00 | 190.14 |
| 2845 | CB | GLU | A | 163 | −9.037 | 37.041 | −10.704 | 1.00 | 249.69 |
| 2846 | CG | GLU | A | 163 | −8.981 | 36.372 | −12.079 | 1.00 | 249.69 |
| 2847 | CD | GLU | A | 163 | −10.356 | 36.015 | −12.616 | 1.00 | 249.69 |
| 2848 | OE1 | GLU | A | 163 | −11.096 | 35.284 | −11.928 | 1.00 | 249.69 |
| 2849 | OE2 | GLU | A | 163 | −10.694 | 36.462 | −13.730 | 1.00 | 249.69 |
| 2850 | C | GLU | A | 163 | −9.764 | 38.962 | −9.264 | 1.00 | 190.14 |
| 2851 | O | GLU | A | 163 | −8.835 | 39.462 | −8.631 | 1.00 | 190.14 |
| 2852 | N | PRO | A | 164 | −10.991 | 38.833 | −8.736 | 1.00 | 116.57 |
| 2853 | CD | PRO | A | 164 | −12.208 | 38.343 | −9.412 | 1.00 | 155.66 |
| 2854 | CA | PRO | A | 164 | −11.302 | 39.267 | −7.368 | 1.00 | 116.57 |
| 2855 | CB | PRO | A | 164 | −12.814 | 39.411 | −7.394 | 1.00 | 155.66 |
| 2856 | CG | PRO | A | 164 | −13.205 | 38.276 | −8.263 | 1.00 | 155.66 |
| 2857 | C | PRO | A | 164 | −10.827 | 38.258 | −6.325 | 1.00 | 116.57 |
| 2858 | O | PRO | A | 164 | −10.826 | 37.056 | −6.578 | 1.00 | 116.57 |
| 2859 | N | LEU | A | 165 | −10.441 | 38.735 | −5.150 | 1.00 | 151.49 |
| 2860 | CA | LEU | A | 165 | −9.949 | 37.832 | −4.122 | 1.00 | 151.49 |
| 2861 | CB | LEU | A | 165 | −8.413 | 37.955 | −4.030 | 1.00 | 116.01 |
| 2862 | CG | LEU | A | 165 | −7.716 | 37.061 | −2.995 | 1.00 | 116.01 |
| 2863 | CD1 | LEU | A | 165 | −8.414 | 35.693 | −2.953 | 1.00 | 116.01 |
| 2864 | CD2 | LEU | A | 165 | −6.244 | 36.924 | −3.335 | 1.00 | 116.01 |
| 2865 | C | LEU | A | 165 | −10.570 | 38.031 | −2.739 | 1.00 | 151.49 |
| 2866 | O | LEU | A | 165 | −10.651 | 39.156 | −2.252 | 1.00 | 151.49 |
| 2867 | N | ASN | A | 166 | −10.996 | 36.932 | −2.113 | 1.00 | 136.62 |
| 2868 | CA | ASN | A | 166 | −11.583 | 36.988 | −0.779 | 1.00 | 136.62 |
| 2869 | CB | ASN | A | 166 | −12.582 | 35.861 | −0.576 | 1.00 | 179.00 |
| 2870 | CG | ASN | A | 166 | −13.998 | 36.278 | −0.898 | 1.00 | 179.00 |
| 2871 | OD1 | ASN | A | 166 | −14.338 | 37.458 | −0.835 | 1.00 | 179.00 |
| 2872 | ND2 | ASN | A | 166 | −14.839 | 35.305 | −1.223 | 1.00 | 179.00 |
| 2873 | C | ASN | A | 166 | −10.535 | 36.890 | 0.307 | 1.00 | 136.62 |
| 2874 | O | ASN | A | 166 | −9.561 | 36.155 | 0.165 | 1.00 | 136.62 |
| 2875 | N | ILE | A | 167 | −10.741 | 37.619 | 1.401 | 1.00 | 136.70 |
| 2876 | CA | ILE | A | 167 | −9.795 | 37.605 | 2.521 | 1.00 | 136.70 |
| 2877 | CB | ILE | A | 167 | −8.857 | 38.814 | 2.472 | 1.00 | 129.99 |
| 2878 | CG2 | ILE | A | 167 | −7.953 | 38.809 | 3.694 | 1.00 | 129.99 |
| 2879 | CG1 | ILE | A | 167 | −8.012 | 38.756 | 1.209 | 1.00 | 129.99 |
| 2880 | CD1 | ILE | A | 167 | −7.114 | 39.934 | 1.059 | 1.00 | 129.99 |
| 2881 | C | ILE | A | 167 | −10.496 | 37.621 | 3.867 | 1.00 | 136.70 |
| 2882 | O | ILE | A | 167 | −11.317 | 38.494 | 4.126 | 1.00 | 136.70 |
| 2883 | N | THR | A | 168 | −10.148 | 36.685 | 4.738 | 1.00 | 125.45 |
| 2884 | CA | THR | A | 168 | −10.808 | 36.661 | 6.016 | 1.00 | 125.45 |
| 2885 | CB | THR | A | 168 | −11.677 | 35.424 | 6.131 | 1.00 | 120.45 |
| 2886 | OG1 | THR | A | 168 | −12.602 | 35.394 | 5.041 | 1.00 | 120.45 |
| 2887 | CG2 | THR | A | 168 | −12.451 | 35.445 | 7.433 | 1.00 | 120.45 |
| 2888 | C | THR | A | 168 | −9.910 | 36.749 | 7.232 | 1.00 | 125.45 |
| 2889 | O | THR | A | 168 | −8.989 | 35.928 | 7.431 | 1.00 | 125.45 |
| 2890 | N | VAL | A | 169 | −10.194 | 37.757 | 8.050 | 1.00 | 104.30 |
| 2891 | CA | VAL | A | 169 | −9.444 | 37.979 | 9.276 | 1.00 | 104.30 |
| 2892 | CB | VAL | A | 169 | −9.061 | 39.471 | 9.422 | 1.00 | 108.18 |
| 2893 | CG1 | VAL | A | 169 | −8.798 | 39.826 | 10.864 | 1.00 | 108.18 |
| 2894 | CG2 | VAL | A | 169 | −7.798 | 39.750 | 8.602 | 1.00 | 108.18 |
| 2895 | C | VAL | A | 169 | −10.333 | 37.534 | 10.433 | 1.00 | 104.30 |
| 2896 | O | VAL | A | 169 | −11.331 | 38.182 | 10.729 | 1.00 | 104.30 |
| 2897 | N | ILE | A | 170 | −9.972 | 36.416 | 11.069 | 1.00 | 193.03 |
| 2898 | CA | ILE | A | 170 | −10.727 | 35.860 | 12.191 | 1.00 | 193.03 |
| 2899 | CB | ILE | A | 170 | −10.701 | 34.321 | 12.132 | 1.00 | 177.52 |
| 2900 | CG2 | ILE | A | 170 | −11.108 | 33.853 | 10.743 | 1.00 | 177.52 |
| 2901 | CG1 | ILE | A | 170 | −9.291 | 33.800 | 12.404 | 1.00 | 177.52 |
| 2902 | CD1 | ILE | A | 170 | −9.174 | 32.283 | 12.422 | 1.00 | 177.52 |
| 2903 | C | ILE | A | 170 | −10.133 | 36.337 | 13.513 | 1.00 | 193.03 |
| 2904 | O | ILE | A | 170 | −9.014 | 36.850 | 13.542 | 1.00 | 193.03 |
| 2905 | N | LYS | A | 171 | −10.858 | 36.172 | 14.614 | 1.00 | 156.56 |
| 2906 | CA | LYS | A | 171 | −10.335 | 36.621 | 15.921 | 1.00 | 156.56 |
| 2907 | CB | LYS | A | 171 | −11.244 | 37.705 | 16.466 | 1.00 | 223.67 |
| 2908 | CG | LYS | A | 171 | −12.675 | 37.247 | 16.553 | 1.00 | 223.67 |
| 2909 | CD | LYS | A | 171 | −13.639 | 38.405 | 16.449 | 1.00 | 223.67 |
| 2910 | CE | LYS | A | 171 | −13.412 | 39.423 | 17.548 | 1.00 | 223.67 |
| 2911 | NZ | LYS | A | 171 | −14.415 | 40.529 | 17.484 | 1.00 | 223.67 |
| 2912 | C | LYS | A | 171 | −10.169 | 35.507 | 16.984 | 1.00 | 156.56 |
| 2913 | O | LYS | A | 171 | −9.911 | 35.776 | 18.164 | 1.00 | 156.56 |
| 2914 | C1 | NAG | A | 221 | 13.115 | 30.531 | −12.704 | 1.00 | 229.93 |
| 2915 | C2 | NAG | A | 221 | 13.292 | 32.012 | −13.027 | 1.00 | 229.93 |
| 2916 | N2 | NAG | A | 221 | 11.991 | 32.635 | −13.150 | 1.00 | 229.93 |
| 2917 | C7 | NAG | A | 221 | 11.855 | 33.943 | −12.957 | 1.00 | 229.93 |
| 2918 | O7 | NAG | A | 221 | 12.801 | 34.685 | −12.679 | 1.00 | 229.93 |
| 2919 | C8 | NAG | A | 221 | 10.451 | 34.518 | −13.100 | 1.00 | 229.93 |
| 2920 | C3 | NAG | A | 221 | 14.066 | 32.185 | −14.322 | 1.00 | 229.93 |
| 2921 | O3 | NAG | A | 221 | 14.354 | 33.560 | −14.516 | 1.00 | 229.93 |
| 2922 | C4 | NAG | A | 221 | 15.380 | 31.386 | −14.319 | 1.00 | 229.93 |
| 2923 | O4 | NAG | A | 221 | 15.903 | 31.411 | −15.666 | 1.00 | 229.93 |
| 2924 | C5 | NAG | A | 221 | 15.121 | 29.925 | −13.874 | 1.00 | 229.93 |
| 2925 | O5 | NAG | A | 221 | 14.399 | 29.895 | −12.623 | 1.00 | 229.93 |
| 2926 | C6 | NAG | A | 221 | 16.390 | 29.114 | −13.656 | 1.00 | 229.93 |
| 2927 | O6 | NAG | A | 221 | 17.244 | 29.725 | −12.701 | 1.00 | 229.93 |
| 2928 | C1 | NAG | A | 222 | 17.240 | 31.098 | −15.903 | 1.00 | 249.69 |
| 2929 | C2 | NAG | A | 222 | 17.830 | 32.101 | −16.914 | 1.00 | 249.69 |
| 2930 | N2 | NAG | A | 222 | 17.769 | 33.452 | −16.374 | 1.00 | 249.69 |
| 2931 | C7 | NAG | A | 222 | 18.879 | 34.168 | −16.205 | 1.00 | 249.69 |
| 2932 | O7 | NAG | A | 222 | 20.003 | 33.746 | −16.487 | 1.00 | 249.69 |
| 2933 | C8 | NAG | A | 222 | 18.718 | 35.569 | −15.634 | 1.00 | 249.69 |
| 2934 | C3 | NAG | A | 222 | 17.038 | 32.023 | −18.236 | 1.00 | 249.69 |
| 2935 | O3 | NAG | A | 222 | 17.639 | 32.861 | −19.217 | 1.00 | 249.69 |
| 2936 | C4 | NAG | A | 222 | 16.979 | 30.571 | −18.752 | 1.00 | 249.69 |
| 2937 | O4 | NAG | A | 222 | 16.114 | 30.501 | −19.878 | 1.00 | 249.69 |
| 2938 | C5 | NAG | A | 222 | 16.463 | 29.634 | −17.646 | 1.00 | 249.69 |
| 2939 | O5 | NAG | A | 222 | 17.286 | 29.769 | −16.459 | 1.00 | 249.69 |
| 2940 | C6 | NAG | A | 222 | 16.462 | 28.165 | −18.038 | 1.00 | 249.69 |
| 2941 | O6 | NAG | A | 222 | 15.210 | 27.555 | −17.749 | 1.00 | 249.69 |
| 2942 | C1 | NAG | A | 242 | −3.871 | 18.493 | −8.371 | 1.00 | 249.50 |
| 2943 | C2 | NAG | A | 242 | −3.270 | 18.370 | −9.775 | 1.00 | 249.50 |
| 2944 | N2 | NAG | A | 242 | −1.860 | 18.040 | −9.718 | 1.00 | 249.50 |
| 2945 | C7 | NAG | A | 242 | −1.426 | 16.919 | −10.287 | 1.00 | 249.50 |
| 2946 | O7 | NAG | A | 242 | −2.178 | 16.128 | −10.862 | 1.00 | 249.50 |
| 2947 | C8 | NAG | A | 242 | 0.063 | 16.621 | −10.205 | 1.00 | 249.50 |
| 2948 | C3 | NAG | A | 242 | −3.480 | 19.691 | −10.511 | 1.00 | 249.50 |
| 2949 | O3 | NAG | A | 242 | −2.951 | 19.600 | −11.829 | 1.00 | 249.50 |
| 2950 | C4 | NAG | A | 242 | −4.979 | 20.019 | −10.567 | 1.00 | 249.50 |
| 2951 | O4 | NAG | A | 242 | −5.159 | 21.345 | −11.115 | 1.00 | 249.50 |
| 2952 | C5 | NAG | A | 242 | −5.622 | 19.952 | −9.158 | 1.00 | 249.50 |
| 2953 | O5 | NAG | A | 242 | −5.285 | 18.711 | −8.481 | 1.00 | 249.50 |
| 2954 | C6 | NAG | A | 242 | −7.140 | 20.004 | −9.235 | 1.00 | 249.50 |
| 2955 | O6 | NAG | A | 242 | −7.650 | 21.225 | −8.725 | 1.00 | 249.50 |
| 2956 | C1 | NAG | A | 243 | −5.905 | 21.444 | −12.280 | 1.00 | 249.69 |
| 2957 | C2 | NAG | A | 243 | −6.423 | 22.875 | −12.441 | 1.00 | 249.69 |
| 2958 | N2 | NAG | A | 243 | −7.258 | 23.262 | −11.323 | 1.00 | 249.69 |
| 2959 | C7 | NAG | A | 243 | −7.047 | 24.432 | −10.724 | 1.00 | 249.69 |
| 2960 | O7 | NAG | A | 243 | −6.150 | 25.215 | −11.062 | 1.00 | 249.69 |
| 2961 | C8 | NAG | A | 243 | −7.964 | 24.787 | −9.565 | 1.00 | 249.69 |
| 2962 | C3 | NAG | A | 243 | −7.210 | 22.971 | −13.739 | 1.00 | 249.69 |
| 2963 | O3 | NAG | A | 243 | −7.711 | 24.291 | −13.913 | 1.00 | 249.69 |
| 2964 | C4 | NAG | A | 243 | −6.286 | 22.613 | −14.888 | 1.00 | 249.69 |
| 2965 | O4 | NAG | A | 243 | −7.053 | 22.760 | −16.068 | 1.00 | 249.69 |
| 2966 | C5 | NAG | A | 243 | −5.731 | 21.178 | −14.681 | 1.00 | 249.69 |
| 2967 | O5 | NAG | A | 243 | −5.049 | 21.113 | −13.392 | 1.00 | 249.69 |
| 2968 | C6 | NAG | A | 243 | −4.717 | 20.769 | −15.727 | 1.00 | 249.69 |
| 2969 | O6 | NAG | A | 243 | −3.570 | 21.598 | −15.679 | 1.00 | 249.69 |
| 2970 | C1 | MAN | A | 244 | −6.484 | 23.132 | −17.256 | 1.00 | 249.69 |
| 2971 | C2 | MAN | A | 244 | −7.225 | 22.291 | −18.199 | 1.00 | 249.69 |
| 2972 | O2 | MAN | A | 244 | −8.623 | 22.309 | −17.842 | 1.00 | 249.69 |
| 2973 | C3 | MAN | A | 244 | −6.903 | 22.695 | −19.610 | 1.00 | 249.69 |
| 2974 | O3 | MAN | A | 244 | −7.502 | 21.811 | −20.538 | 1.00 | 249.69 |
| 2975 | C4 | MAN | A | 244 | −7.252 | 24.155 | −19.854 | 1.00 | 249.69 |
| 2976 | O4 | MAN | A | 244 | −6.977 | 24.497 | −21.200 | 1.00 | 249.69 |
| 2977 | C5 | MAN | A | 244 | −6.404 | 24.996 | −18.895 | 1.00 | 249.69 |
| 2978 | O5 | MAN | A | 244 | −6.748 | 24.610 | −17.507 | 1.00 | 249.69 |
| 2979 | C6 | MAN | A | 244 | −6.499 | 26.518 | −19.137 | 1.00 | 249.69 |
| 2980 | O6 | MAN | A | 244 | −7.631 | 27.105 | −18.519 | 1.00 | 249.69 |
| 2981 | C1 | NAG | A | 250 | 17.983 | 21.117 | −1.207 | 1.00 | 249.69 |
| 2982 | C2 | NAG | A | 250 | 19.036 | 22.142 | −0.738 | 1.00 | 249.69 |
| 2983 | N2 | NAG | A | 250 | 19.037 | 22.235 | 0.709 | 1.00 | 249.69 |
| 2984 | C7 | NAG | A | 250 | 20.062 | 21.752 | 1.406 | 1.00 | 249.69 |
| 2985 | O7 | NAG | A | 250 | 21.042 | 21.214 | 0.876 | 1.00 | 249.69 |
| 2986 | C8 | NAG | A | 250 | 19.990 | 21.883 | 2.924 | 1.00 | 249.69 |
| 2987 | C3 | NAG | A | 250 | 18.721 | 23.516 | −1.350 | 1.00 | 249.69 |
| 2988 | O3 | NAG | A | 250 | 19.736 | 24.449 | −1.001 | 1.00 | 249.69 |
| 2989 | C4 | NAG | A | 250 | 18.617 | 23.400 | −2.878 | 1.00 | 249.69 |
| 2990 | O4 | NAG | A | 250 | 18.193 | 24.648 | −3.416 | 1.00 | 249.69 |
| 2991 | C5 | NAG | A | 250 | 17.612 | 22.286 | −3.261 | 1.00 | 249.69 |
| 2992 | O5 | NAG | A | 250 | 17.986 | 21.032 | −2.636 | 1.00 | 249.69 |
| 2993 | C6 | NAG | A | 250 | 17.526 | 22.023 | −4.759 | 1.00 | 249.69 |
| 2994 | O6 | NAG | A | 250 | 16.887 | 20.775 | −5.028 | 1.00 | 249.69 |
| 2995 | C1 | NAG | A | 274 | 0.355 | 12.405 | 15.723 | 1.00 | 249.69 |
| 2996 | C2 | NAG | A | 274 | −0.462 | 13.289 | 16.690 | 1.00 | 249.69 |
| 2997 | N2 | NAG | A | 274 | 0.423 | 14.159 | 17.448 | 1.00 | 249.69 |
| 2998 | C7 | NAG | A | 274 | −0.025 | 15.313 | 17.944 | 1.00 | 249.69 |
| 2999 | O7 | NAG | A | 274 | −1.188 | 15.702 | 17.799 | 1.00 | 249.69 |
| 3000 | C8 | NAG | A | 274 | 0.966 | 16.171 | 18.721 | 1.00 | 249.69 |
| 3001 | C3 | NAG | A | 274 | −1.276 | 12.407 | 17.651 | 1.00 | 249.69 |
| 3002 | O3 | NAG | A | 274 | −2.130 | 13.222 | 18.443 | 1.00 | 249.69 |
| 3003 | C4 | NAG | A | 274 | −2.117 | 11.387 | 16.868 | 1.00 | 249.69 |
| 3004 | O4 | NAG | A | 274 | −2.765 | 10.493 | 17.768 | 1.00 | 249.69 |
| 3005 | C5 | NAG | A | 274 | −1.221 | 10.596 | 15.901 | 1.00 | 249.69 |
| 3006 | O5 | NAG | A | 274 | −0.517 | 11.505 | 15.017 | 1.00 | 249.69 |
| 3007 | C6 | NAG | A | 274 | −2.018 | 9.637 | 15.026 | 1.00 | 249.69 |
| 3008 | O6 | NAG | A | 274 | −1.206 | 9.058 | 14.010 | 1.00 | 249.69 |
| 3009 | C1 | NAG | A | 335 | 5.793 | 44.302 | −4.488 | 1.00 | 249.69 |
| 3010 | C2 | NAG | A | 335 | 6.924 | 43.869 | −3.512 | 1.00 | 249.69 |
| 3011 | N2 | NAG | A | 335 | 6.696 | 44.490 | −2.220 | 1.00 | 249.69 |
| 3012 | C7 | NAG | A | 335 | 6.442 | 43.744 | −1.148 | 1.00 | 249.69 |
| 3013 | O7 | NAG | A | 335 | 6.394 | 42.513 | −1.175 | 1.00 | 249.69 |
| 3014 | C8 | NAG | A | 335 | 6.211 | 44.481 | 0.156 | 1.00 | 249.69 |
| 3015 | C3 | NAG | A | 335 | 8.352 | 44.222 | −3.983 | 1.00 | 249.69 |
| 3016 | O3 | NAG | A | 335 | 9.296 | 43.421 | −3.281 | 1.00 | 249.69 |
| 3017 | C4 | NAG | A | 335 | 8.520 | 43.993 | −5.483 | 1.00 | 249.69 |
| 3018 | O4 | NAG | A | 335 | 9.821 | 44.401 | −5.897 | 1.00 | 249.69 |
| 3019 | C5 | NAG | A | 335 | 7.450 | 44.802 | −6.205 | 1.00 | 249.69 |
| 3020 | O5 | NAG | A | 335 | 6.149 | 44.255 | −5.895 | 1.00 | 249.69 |
| 3021 | C6 | NAG | A | 335 | 7.609 | 44.762 | −7.718 | 1.00 | 249.69 |
| 3022 | O6 | NAG | A | 335 | 7.688 | 46.071 | −8.267 | 1.00 | 249.69 |
| 3023 | C1 | NAG | A | 340 | −3.087 | 46.639 | 17.035 | 1.00 | 249.69 |
| 3024 | C2 | NAG | A | 340 | −3.935 | 45.839 | 18.030 | 1.00 | 249.69 |
| 3025 | N2 | NAG | A | 340 | −4.856 | 44.975 | 17.311 | 1.00 | 249.69 |
| 3026 | C7 | NAG | A | 340 | −4.995 | 43.695 | 17.659 | 1.00 | 249.69 |
| 3027 | O7 | NAG | A | 340 | −4.379 | 43.170 | 18.595 | 1.00 | 249.69 |
| 3028 | C8 | NAG | A | 340 | −5.979 | 42.868 | 16.843 | 1.00 | 249.69 |
| 3029 | C3 | NAG | A | 340 | −4.707 | 46.820 | 18.927 | 1.00 | 249.69 |
| 3030 | O3 | NAG | A | 340 | −5.434 | 46.110 | 19.924 | 1.00 | 249.69 |
| 3031 | C4 | NAG | A | 340 | −3.738 | 47.802 | 19.596 | 1.00 | 249.69 |
| 3032 | O4 | NAG | A | 340 | −4.485 | 48.790 | 20.299 | 1.00 | 249.69 |
| 3033 | C5 | NAG | A | 340 | −2.841 | 48.473 | 18.533 | 1.00 | 249.69 |
| 3034 | O5 | NAG | A | 340 | −2.166 | 47.471 | 17.739 | 1.00 | 249.69 |
| 3035 | C6 | NAG | A | 340 | −1.761 | 49.371 | 19.114 | 1.00 | 249.69 |
| 3036 | O6 | NAG | A | 340 | −0.846 | 49.785 | 18.103 | 1.00 | 249.69 |
| 3037 | C1 | NAG | A | 366 | −16.179 | 35.618 | −1.670 | 1.00 | 221.62 |
| 3038 | C2 | NAG | A | 366 | −16.600 | 34.642 | −2.761 | 1.00 | 221.62 |
| 3039 | N2 | NAG | A | 366 | −15.672 | 34.736 | −3.871 | 1.00 | 221.62 |
| 3040 | C7 | NAG | A | 366 | −14.602 | 33.944 | −3.922 | 1.00 | 221.62 |
| 3041 | O7 | NAG | A | 366 | −14.351 | 33.096 | −3.062 | 1.00 | 221.62 |
| 3042 | C8 | NAG | A | 366 | −13.672 | 34.112 | −5.114 | 1.00 | 221.62 |
| 3043 | C3 | NAG | A | 366 | −18.011 | 34.981 | −3.236 | 1.00 | 221.62 |
| 3044 | O3 | NAG | A | 366 | −18.470 | 33.973 | −4.125 | 1.00 | 221.62 |
| 3045 | C4 | NAG | A | 366 | −18.991 | 35.113 | −2.065 | 1.00 | 221.62 |
| 3046 | O4 | NAG | A | 366 | −20.223 | 35.683 | −2.557 | 1.00 | 221.62 |
| 3047 | C5 | NAG | A | 366 | −18.409 | 36.017 | −0.964 | 1.00 | 221.62 |
| 3048 | O5 | NAG | A | 366 | −17.100 | 35.560 | −0.585 | 1.00 | 221.62 |
| 3049 | C6 | NAG | A | 366 | −19.246 | 36.056 | 0.304 | 1.00 | 221.62 |
| 3050 | O6 | NAG | A | 366 | −18.758 | 37.042 | 1.205 | 1.00 | 221.62 |
| 3051 | C1 | NAG | A | 367 | −21.391 | 34.987 | −2.286 | 1.00 | 249.69 |
| 3052 | C2 | NAG | A | 367 | −22.592 | 35.932 | −2.385 | 1.00 | 249.69 |
| 3053 | N2 | NAG | A | 367 | −22.437 | 37.053 | −1.478 | 1.00 | 249.69 |
| 3054 | C7 | NAG | A | 367 | −22.260 | 38.275 | −1.969 | 1.00 | 249.69 |
| 3055 | O7 | NAG | A | 367 | −22.222 | 38.512 | −3.181 | 1.00 | 249.69 |
| 3056 | C8 | NAG | A | 367 | −22.101 | 39.407 | −0.966 | 1.00 | 249.69 |
| 3057 | C3 | NAG | A | 367 | −23.858 | 35.142 | −2.058 | 1.00 | 249.69 |
| 3058 | O3 | NAG | A | 367 | −24.998 | 35.986 | −2.163 | 1.00 | 249.69 |
| 3059 | C4 | NAG | A | 367 | −23.984 | 33.958 | −3.031 | 1.00 | 249.69 |
| 3060 | O4 | NAG | A | 367 | −25.101 | 33.153 | −2.664 | 1.00 | 249.69 |
| 3061 | C5 | NAG | A | 367 | −22.694 | 33.108 | −3.015 | 1.00 | 249.69 |
| 3062 | O5 | NAG | A | 367 | −21.528 | 33.938 | −3.257 | 1.00 | 249.69 |
| 3063 | C6 | NAG | A | 367 | −22.696 | 32.031 | −4.083 | 1.00 | 249.69 |
| 3064 | O6 | NAG | A | 367 | −21.707 | 32.291 | −5.071 | 1.00 | 249.69 |
| 3065 | CB | LYS | B | 4 | 31.112 | 63.164 | 23.840 | 1.00 | 249.69 |
| 3066 | CG | LYS | B | 4 | 31.172 | 64.583 | 23.260 | 1.00 | 249.69 |
| 3067 | CD | LYS | B | 4 | 31.232 | 65.658 | 24.353 | 1.00 | 249.69 |
| 3068 | CE | LYS | B | 4 | 31.339 | 67.065 | 23.748 | 1.00 | 249.69 |
| 3069 | NZ | LYS | B | 4 | 31.384 | 68.141 | 24.779 | 1.00 | 249.69 |
| 3070 | C | LYS | B | 4 | 32.410 | 62.191 | 21.928 | 1.00 | 249.69 |
| 3071 | O | LYS | B | 4 | 33.409 | 62.759 | 22.371 | 1.00 | 249.69 |
| 3072 | N | LYS | B | 4 | 31.072 | 60.721 | 23.434 | 1.00 | 249.69 |
| 3073 | CA | LYS | B | 4 | 31.141 | 62.056 | 22.777 | 1.00 | 249.69 |
| 3074 | N | PRO | B | 5 | 32.389 | 61.656 | 20.698 | 1.00 | 223.70 |
| 3075 | CD | PRO | B | 5 | 31.376 | 60.752 | 20.128 | 1.00 | 195.56 |
| 3076 | CA | PRO | B | 5 | 33.562 | 61.750 | 19.825 | 1.00 | 223.70 |
| 3077 | CB | PRO | B | 5 | 33.387 | 60.550 | 18.906 | 1.00 | 195.56 |
| 3078 | CG | PRO | B | 5 | 31.904 | 60.498 | 18.724 | 1.00 | 195.56 |
| 3079 | C | PRO | B | 5 | 33.598 | 63.077 | 19.060 | 1.00 | 223.70 |
| 3080 | O | PRO | B | 5 | 32.576 | 63.741 | 18.895 | 1.00 | 223.70 |
| 3081 | N | LYS | B | 6 | 34.780 | 63.472 | 18.605 | 1.00 | 208.07 |
| 3082 | CA | LYS | B | 6 | 34.916 | 64.713 | 17.858 | 1.00 | 208.07 |
| 3083 | CB | LYS | B | 6 | 35.357 | 65.849 | 18.783 | 1.00 | 245.96 |
| 3084 | CG | LYS | B | 6 | 35.467 | 67.195 | 18.074 | 1.00 | 245.96 |
| 3085 | CD | LYS | B | 6 | 35.756 | 68.337 | 19.039 | 1.00 | 245.96 |
| 3086 | CE | LYS | B | 6 | 35.816 | 69.677 | 18.304 | 1.00 | 245.96 |
| 3087 | NZ | LYS | B | 6 | 35.973 | 70.834 | 19.231 | 1.00 | 245.96 |
| 3088 | C | LYS | B | 6 | 35.920 | 64.543 | 16.721 | 1.00 | 208.07 |
| 3089 | O | LYS | B | 6 | 37.122 | 64.371 | 16.946 | 1.00 | 208.07 |
| 3090 | N | VAL | B | 7 | 35.414 | 64.603 | 15.497 | 1.00 | 211.91 |
| 3091 | CA | VAL | B | 7 | 36.245 | 64.443 | 14.312 | 1.00 | 211.91 |
| 3092 | CB | VAL | B | 7 | 35.379 | 64.356 | 13.053 | 1.00 | 105.28 |
| 3093 | CG1 | VAL | B | 7 | 36.218 | 63.818 | 11.881 | 1.00 | 105.28 |
| 3094 | CG2 | VAL | B | 7 | 34.151 | 63.495 | 13.325 | 1.00 | 105.28 |
| 3095 | C | VAL | B | 7 | 37.258 | 65.565 | 14.107 | 1.00 | 211.91 |
| 3096 | O | VAL | B | 7 | 36.903 | 66.741 | 14.033 | 1.00 | 211.91 |
| 3097 | N | SER | B | 8 | 38.524 | 65.185 | 14.009 | 1.00 | 208.76 |
| 3098 | CA | SER | B | 8 | 39.599 | 66.141 | 13.797 | 1.00 | 208.76 |
| 3099 | CB | SER | B | 8 | 40.749 | 65.846 | 14.770 | 1.00 | 216.01 |
| 3100 | OG | SER | B | 8 | 41.085 | 64.465 | 14.777 | 1.00 | 216.01 |
| 3101 | C | SER | B | 8 | 40.084 | 66.033 | 12.348 | 1.00 | 208.76 |
| 3102 | O | SER | B | 8 | 39.830 | 65.031 | 11.685 | 1.00 | 208.76 |
| 3103 | N | LEU | B | 9 | 40.767 | 67.061 | 11.853 | 1.00 | 194.89 |
| 3104 | CA | LEU | B | 9 | 41.278 | 67.030 | 10.487 | 1.00 | 194.89 |
| 3105 | CB | LEU | B | 9 | 40.528 | 68.017 | 9.599 | 1.00 | 159.88 |
| 3106 | CG | LEU | B | 9 | 39.017 | 67.914 | 9.401 | 1.00 | 159.88 |
| 3107 | CD1 | LEU | B | 9 | 38.658 | 68.762 | 8.190 | 1.00 | 159.88 |
| 3108 | CD2 | LEU | B | 9 | 38.575 | 66.489 | 9.175 | 1.00 | 159.88 |
| 3109 | C | LEU | B | 9 | 42.760 | 67.363 | 10.413 | 1.00 | 194.89 |
| 3110 | O | LEU | B | 9 | 43.318 | 67.981 | 11.315 | 1.00 | 194.89 |
| 3111 | N | ASN | B | 10 | 43.390 | 66.956 | 9.319 | 1.00 | 186.22 |
| 3112 | CA | ASN | B | 10 | 44.801 | 67.229 | 9.113 | 1.00 | 186.22 |
| 3113 | CB | ASN | B | 10 | 45.653 | 66.253 | 9.914 | 1.00 | 231.85 |
| 3114 | CG | ASN | B | 10 | 47.090 | 66.704 | 10.015 | 1.00 | 231.85 |
| 3115 | OD1 | ASN | B | 10 | 47.381 | 67.753 | 10.592 | 1.00 | 231.85 |
| 3116 | ND2 | ASN | B | 10 | 47.999 | 65.921 | 9.445 | 1.00 | 231.85 |
| 3117 | C | ASN | B | 10 | 45.157 | 67.123 | 7.638 | 1.00 | 186.22 |
| 3118 | O | ASN | B | 10 | 45.137 | 66.030 | 7.059 | 1.00 | 186.22 |
| 3119 | N | PRO | B | 11 | 45.503 | 68.262 | 6.991 | 1.00 | 188.99 |
| 3120 | CD | PRO | B | 11 | 45.868 | 68.247 | 5.571 | 1.00 | 219.87 |
| 3121 | CA | PRO | B | 11 | 45.592 | 69.622 | 7.539 | 1.00 | 188.99 |
| 3122 | CB | PRO | B | 11 | 45.872 | 70.458 | 6.284 | 1.00 | 219.87 |
| 3123 | CG | PRO | B | 11 | 46.650 | 69.526 | 5.431 | 1.00 | 219.87 |
| 3124 | C | PRO | B | 11 | 44.336 | 70.101 | 8.267 | 1.00 | 188.99 |
| 3125 | O | PRO | B | 11 | 43.282 | 69.480 | 8.188 | 1.00 | 188.99 |
| 3126 | N | PRO | B | 12 | 44.443 | 71.222 | 9.003 | 1.00 | 172.73 |
| 3127 | CD | PRO | B | 12 | 45.661 | 71.989 | 9.311 | 1.00 | 135.97 |
| 3128 | CA | PRO | B | 12 | 43.290 | 71.755 | 9.739 | 1.00 | 172.73 |
| 3129 | CB | PRO | B | 12 | 43.920 | 72.837 | 10.623 | 1.00 | 135.97 |
| 3130 | CG | PRO | B | 12 | 45.375 | 72.451 | 10.702 | 1.00 | 135.97 |
| 3131 | C | PRO | B | 12 | 42.274 | 72.344 | 8.744 | 1.00 | 172.73 |
| 3132 | O | PRO | B | 12 | 41.079 | 72.406 | 9.017 | 1.00 | 172.73 |
| 3133 | N | TRP | B | 13 | 42.788 | 72.770 | 7.593 | 1.00 | 154.67 |
| 3134 | CA | TRP | B | 13 | 42.024 | 73.371 | 6.503 | 1.00 | 154.67 |
| 3135 | CB | TRP | B | 13 | 42.952 | 73.560 | 5.300 | 1.00 | 165.26 |
| 3136 | CG | TRP | B | 13 | 44.249 | 74.215 | 5.658 | 1.00 | 165.26 |
| 3137 | CD2 | TRP | B | 13 | 44.455 | 75.194 | 6.673 | 1.00 | 165.26 |
| 3138 | CE2 | TRP | B | 13 | 45.818 | 75.545 | 6.650 | 1.00 | 165.26 |
| 3139 | CE3 | TRP | B | 13 | 43.612 | 75.818 | 7.601 | 1.00 | 165.26 |
| 3140 | CD1 | TRP | B | 13 | 45.469 | 74.009 | 5.073 | 1.00 | 165.26 |
| 3141 | NE1 | TRP | B | 13 | 46.419 | 74.806 | 5.668 | 1.00 | 165.26 |
| 3142 | CZ2 | TRP | B | 13 | 46.356 | 76.486 | 7.521 | 1.00 | 165.26 |
| 3143 | CZ3 | TRP | B | 13 | 44.146 | 76.753 | 8.464 | 1.00 | 165.26 |
| 3144 | CH2 | TRP | B | 13 | 45.506 | 77.080 | 8.420 | 1.00 | 165.26 |
| 3145 | C | TRP | B | 13 | 40.840 | 72.520 | 6.088 | 1.00 | 154.67 |
| 3146 | O | TRP | B | 13 | 41.023 | 71.414 | 5.593 | 1.00 | 154.67 |
| 3147 | N | ASN | B | 14 | 39.627 | 73.038 | 6.265 | 1.00 | 126.91 |
| 3148 | CA | ASN | B | 14 | 38.416 | 72.290 | 5.885 | 1.00 | 126.91 |
| 3149 | CB | ASN | B | 14 | 37.397 | 72.308 | 7.035 | 1.00 | 191.01 |
| 3150 | CG | ASN | B | 14 | 36.877 | 73.687 | 7.331 | 1.00 | 191.01 |
| 3151 | OD1 | ASN | B | 14 | 37.643 | 74.604 | 7.635 | 1.00 | 191.01 |
| 3152 | ND2 | ASN | B | 14 | 35.564 | 73.848 | 7.246 | 1.00 | 191.01 |
| 3153 | C | ASN | B | 14 | 37.778 | 72.796 | 4.590 | 1.00 | 126.91 |
| 3154 | O | ASN | B | 14 | 36.595 | 72.577 | 4.336 | 1.00 | 126.91 |
| 3155 | N | ARG | B | 15 | 38.606 | 73.477 | 3.799 | 1.00 | 109.47 |
| 3156 | CA | ARG | B | 15 | 38.240 | 74.033 | 2.509 | 1.00 | 109.47 |
| 3157 | CB | ARG | B | 15 | 38.096 | 75.576 | 2.571 | 1.00 | 119.97 |
| 3158 | CG | ARG | B | 15 | 37.202 | 76.153 | 3.671 | 1.00 | 119.97 |
| 3159 | CD | ARG | B | 15 | 37.010 | 77.677 | 3.482 | 1.00 | 119.97 |
| 3160 | NE | ARG | B | 15 | 36.034 | 78.008 | 2.444 | 1.00 | 119.97 |
| 3161 | CZ | ARG | B | 15 | 36.135 | 79.040 | 1.615 | 1.00 | 119.97 |
| 3162 | NH1 | ARG | B | 15 | 37.176 | 79.846 | 1.693 | 1.00 | 119.97 |
| 3163 | NH2 | ARG | B | 15 | 35.187 | 79.273 | 0.715 | 1.00 | 119.97 |
| 3164 | C | ARG | B | 15 | 39.432 | 73.688 | 1.616 | 1.00 | 109.47 |
| 3165 | O | ARG | B | 15 | 40.462 | 74.353 | 1.689 | 1.00 | 109.47 |
| 3166 | N | ILE | B | 16 | 39.307 | 72.675 | 0.769 | 1.00 | 122.07 |
| 3167 | CA | ILE | B | 16 | 40.431 | 72.294 | −0.072 | 1.00 | 122.07 |
| 3168 | CB | ILE | B | 16 | 40.914 | 70.905 | 0.303 | 1.00 | 121.17 |
| 3169 | CG2 | ILE | B | 16 | 41.691 | 70.954 | 1.608 | 1.00 | 121.17 |
| 3170 | CG1 | ILE | B | 16 | 39.708 | 69.975 | 0.377 | 1.00 | 121.17 |
| 3171 | CD1 | ILE | B | 16 | 40.058 | 68.526 | 0.495 | 1.00 | 121.17 |
| 3172 | C | ILE | B | 16 | 40.206 | 72.279 | −1.571 | 1.00 | 122.07 |
| 3173 | O | ILE | B | 16 | 39.087 | 72.146 | −2.041 | 1.00 | 122.07 |
| 3174 | N | PHE | B | 17 | 41.299 | 72.390 | −2.314 | 1.00 | 169.19 |
| 3175 | CA | PHE | B | 17 | 41.255 | 72.361 | −3.770 | 1.00 | 169.19 |
| 3176 | CB | PHE | B | 17 | 42.595 | 72.815 | −4.351 | 1.00 | 156.59 |
| 3177 | CG | PHE | B | 17 | 42.685 | 74.285 | −4.609 | 1.00 | 156.59 |
| 3178 | CD1 | PHE | B | 17 | 43.888 | 74.960 | −4.420 | 1.00 | 156.59 |
| 3179 | CD2 | PHE | B | 17 | 41.586 | 74.990 | −5.076 | 1.00 | 156.59 |
| 3180 | CE1 | PHE | B | 17 | 43.987 | 76.315 | −4.690 | 1.00 | 156.59 |
| 3181 | CE2 | PHE | B | 17 | 41.684 | 76.350 | −5.349 | 1.00 | 156.59 |
| 3182 | CZ | PHE | B | 17 | 42.886 | 77.013 | −5.156 | 1.00 | 156.59 |
| 3183 | C | PHE | B | 17 | 40.970 | 70.947 | −4.258 | 1.00 | 169.19 |
| 3184 | O | PHE | B | 17 | 40.883 | 70.006 | −3.460 | 1.00 | 169.19 |
| 3185 | N | LYS | B | 18 | 40.853 | 70.811 | −5.575 | 1.00 | 133.30 |
| 3186 | CA | LYS | B | 18 | 40.573 | 69.531 | −6.208 | 1.00 | 133.30 |
| 3187 | CB | LYS | B | 18 | 39.922 | 69.777 | −7.575 | 1.00 | 237.07 |
| 3188 | CG | LYS | B | 18 | 39.500 | 68.537 | −8.339 | 1.00 | 237.07 |
| 3189 | CD | LYS | B | 18 | 38.720 | 68.925 | −9.585 | 1.00 | 237.07 |
| 3190 | CE | LYS | B | 18 | 38.347 | 67.714 | −10.424 | 1.00 | 237.07 |
| 3191 | NZ | LYS | B | 18 | 39.539 | 67.079 | −11.054 | 1.00 | 237.07 |
| 3192 | C | LYS | B | 18 | 41.851 | 68.693 | −6.363 | 1.00 | 133.30 |
| 3193 | O | LYS | B | 18 | 42.864 | 69.166 | −6.884 | 1.00 | 133.30 |
| 3194 | N | GLY | B | 19 | 41.799 | 67.448 | −5.899 | 1.00 | 182.54 |
| 3195 | CA | GLY | B | 19 | 42.942 | 66.562 | −6.017 | 1.00 | 182.54 |
| 3196 | C | GLY | B | 19 | 43.823 | 66.498 | −4.794 | 1.00 | 182.54 |
| 3197 | O | GLY | B | 19 | 44.703 | 65.642 | −4.724 | 1.00 | 182.54 |
| 3198 | N | GLU | B | 20 | 43.591 | 67.391 | −3.835 | 1.00 | 116.49 |
| 3199 | CA | GLU | B | 20 | 44.396 | 67.424 | −2.606 | 1.00 | 116.49 |
| 3200 | CB | GLU | B | 20 | 44.276 | 68.800 | −1.920 | 1.00 | 185.38 |
| 3201 | CG | GLU | B | 20 | 44.484 | 70.019 | −2.822 | 1.00 | 185.38 |
| 3202 | CD | GLU | B | 20 | 44.476 | 71.336 | −2.047 | 1.00 | 185.38 |
| 3203 | OE1 | GLU | B | 20 | 43.513 | 71.585 | −1.288 | 1.00 | 185.38 |
| 3204 | OE2 | GLU | B | 20 | 45.433 | 72.125 | −2.205 | 1.00 | 185.38 |
| 3205 | C | GLU | B | 20 | 43.948 | 66.330 | −1.622 | 1.00 | 116.49 |
| 3206 | O | GLU | B | 20 | 42.816 | 65.854 | −1.729 | 1.00 | 116.49 |
| 3207 | N | ASN | B | 21 | 44.810 | 65.944 | −0.669 | 1.00 | 130.57 |
| 3208 | CA | ASN | B | 21 | 44.430 | 64.911 | 0.300 | 1.00 | 130.57 |
| 3209 | CB | ASN | B | 21 | 45.473 | 63.791 | 0.353 | 1.00 | 248.12 |
| 3210 | CG | ASN | B | 21 | 46.097 | 63.492 | −0.992 | 1.00 | 248.12 |
| 3211 | OD1 | ASN | B | 21 | 45.414 | 63.347 | −2.004 | 1.00 | 248.12 |
| 3212 | ND2 | ASN | B | 21 | 47.420 | 63.384 | −0.980 | 1.00 | 248.12 |
| 3213 | C | ASN | B | 21 | 44.229 | 65.432 | 1.730 | 1.00 | 130.57 |
| 3214 | O | ASN | B | 21 | 44.972 | 66.308 | 2.194 | 1.00 | 130.57 |
| 3215 | N | VAL | B | 22 | 43.243 | 64.865 | 2.428 | 1.00 | 161.15 |
| 3216 | CA | VAL | B | 22 | 42.933 | 65.242 | 3.810 | 1.00 | 161.15 |
| 3217 | CB | VAL | B | 22 | 41.702 | 66.157 | 3.876 | 1.00 | 160.53 |
| 3218 | CG1 | VAL | B | 22 | 40.447 | 65.365 | 3.549 | 1.00 | 160.53 |
| 3219 | CG2 | VAL | B | 22 | 41.584 | 66.778 | 5.249 | 1.00 | 160.53 |
| 3220 | C | VAL | B | 22 | 42.633 | 64.002 | 4.652 | 1.00 | 161.15 |
| 3221 | O | VAL | B | 22 | 42.062 | 63.035 | 4.141 | 1.00 | 161.15 |
| 3222 | N | THR | B | 23 | 42.985 | 64.041 | 5.940 | 1.00 | 186.22 |
| 3223 | CA | THR | B | 23 | 42.771 | 62.898 | 6.849 | 1.00 | 186.22 |
| 3224 | CB | THR | B | 23 | 44.108 | 62.440 | 7.478 | 1.00 | 249.25 |
| 3225 | OG1 | THR | B | 23 | 45.086 | 62.250 | 6.448 | 1.00 | 249.25 |
| 3226 | CG2 | THR | B | 23 | 43.919 | 61.136 | 8.238 | 1.00 | 249.25 |
| 3227 | C | THR | B | 23 | 41.804 | 63.173 | 8.004 | 1.00 | 186.22 |
| 3228 | O | THR | B | 23 | 42.015 | 64.109 | 8.783 | 1.00 | 186.22 |
| 3229 | N | LEU | B | 24 | 40.764 | 62.345 | 8.132 | 1.00 | 183.26 |
| 3230 | CA | LEU | B | 24 | 39.782 | 62.522 | 9.208 | 1.00 | 183.26 |
| 3231 | CB | LEU | B | 24 | 38.339 | 62.398 | 8.677 | 1.00 | 177.01 |
| 3232 | CG | LEU | B | 24 | 37.949 | 62.842 | 7.258 | 1.00 | 177.01 |
| 3233 | CD1 | LEU | B | 24 | 36.435 | 62.918 | 7.183 | 1.00 | 177.01 |
| 3234 | CD2 | LEU | B | 24 | 38.553 | 64.190 | 6.910 | 1.00 | 177.01 |
| 3235 | C | LEU | B | 24 | 39.974 | 61.501 | 10.329 | 1.00 | 183.26 |
| 3236 | O | LEU | B | 24 | 39.661 | 60.321 | 10.162 | 1.00 | 183.26 |
| 3237 | N | THR | B | 25 | 40.476 | 61.965 | 11.472 | 1.00 | 238.46 |
| 3238 | CA | THR | B | 25 | 40.717 | 61.111 | 12.636 | 1.00 | 238.46 |
| 3239 | CB | THR | B | 25 | 42.027 | 61.517 | 13.351 | 1.00 | 207.80 |
| 3240 | OG1 | THR | B | 25 | 43.116 | 61.448 | 12.424 | 1.00 | 207.80 |
| 3241 | CG2 | THR | B | 25 | 42.312 | 60.594 | 14.527 | 1.00 | 207.80 |
| 3242 | C | THR | B | 25 | 39.562 | 61.234 | 13.632 | 1.00 | 238.46 |
| 3243 | O | THR | B | 25 | 39.133 | 62.342 | 13.949 | 1.00 | 238.46 |
| 3244 | N | CYS | B | 26 | 39.069 | 60.099 | 14.126 | 1.00 | 203.48 |
| 3245 | CA | CYS | B | 26 | 37.965 | 60.098 | 15.088 | 1.00 | 203.48 |
| 3246 | C | CYS | B | 26 | 38.484 | 60.356 | 16.500 | 1.00 | 203.48 |
| 3247 | O | CYS | B | 26 | 39.563 | 59.880 | 16.861 | 1.00 | 203.48 |
| 3248 | CB | CYS | B | 26 | 37.227 | 58.767 | 15.036 | 1.00 | 181.40 |
| 3249 | SG | CYS | B | 26 | 35.662 | 58.718 | 15.964 | 1.00 | 181.40 |
| 3250 | N | ASN | B | 27 | 37.708 | 61.100 | 17.294 | 1.00 | 249.69 |
| 3251 | CA | ASN | B | 27 | 38.087 | 61.472 | 18.666 | 1.00 | 249.69 |
| 3252 | CB | ASN | B | 27 | 36.876 | 61.388 | 19.608 | 1.00 | 249.69 |
| 3253 | CG | ASN | B | 27 | 37.148 | 62.023 | 20.969 | 1.00 | 249.69 |
| 3254 | OD1 | ASN | B | 27 | 37.644 | 63.155 | 21.060 | 1.00 | 249.69 |
| 3255 | ND2 | ASN | B | 27 | 36.820 | 61.299 | 22.033 | 1.00 | 249.69 |
| 3256 | C | ASN | B | 27 | 39.259 | 60.677 | 19.254 | 1.00 | 249.69 |
| 3257 | O | ASN | B | 27 | 39.081 | 59.605 | 19.837 | 1.00 | 249.69 |
| 3258 | N | GLY | B | 28 | 40.455 | 61.234 | 19.090 | 1.00 | 244.48 |
| 3259 | CA | GLY | B | 28 | 41.676 | 60.619 | 19.577 | 1.00 | 244.48 |
| 3260 | C | GLY | B | 28 | 42.824 | 61.439 | 19.026 | 1.00 | 244.48 |
| 3261 | O | GLY | B | 28 | 42.970 | 61.561 | 17.809 | 1.00 | 244.48 |
| 3262 | N | ASN | B | 29 | 43.638 | 62.008 | 19.912 | 1.00 | 249.69 |
| 3263 | CA | ASN | B | 29 | 44.763 | 62.853 | 19.497 | 1.00 | 249.69 |
| 3264 | CB | ASN | B | 29 | 45.261 | 63.688 | 20.698 | 1.00 | 249.69 |
| 3265 | CG | ASN | B | 29 | 46.236 | 64.797 | 20.295 | 1.00 | 249.69 |
| 3266 | OD1 | ASN | B | 29 | 46.441 | 65.073 | 19.106 | 1.00 | 249.69 |
| 3267 | ND2 | ASN | B | 29 | 46.830 | 65.444 | 21.293 | 1.00 | 249.69 |
| 3268 | C | ASN | B | 29 | 45.930 | 62.076 | 18.865 | 1.00 | 249.69 |
| 3269 | O | ASN | B | 29 | 46.375 | 62.412 | 17.757 | 1.00 | 249.69 |
| 3270 | N | ASN | B | 30 | 46.412 | 61.034 | 19.543 | 1.00 | 249.69 |
| 3271 | CA | ASN | B | 30 | 47.543 | 60.279 | 19.017 | 1.00 | 249.69 |
| 3272 | CB | ASN | B | 30 | 48.783 | 60.555 | 19.881 | 1.00 | 249.69 |
| 3273 | CG | ASN | B | 30 | 49.224 | 62.017 | 19.831 | 1.00 | 249.69 |
| 3274 | OD1 | ASN | B | 30 | 49.500 | 62.634 | 20.869 | 1.00 | 249.69 |
| 3275 | ND2 | ASN | B | 30 | 49.301 | 62.574 | 18.622 | 1.00 | 249.69 |
| 3276 | C | ASN | B | 30 | 47.341 | 58.772 | 18.864 | 1.00 | 249.69 |
| 3277 | O | ASN | B | 30 | 47.289 | 58.267 | 17.736 | 1.00 | 249.69 |
| 3278 | N | PHE | B | 31 | 47.227 | 58.056 | 19.985 | 1.00 | 249.69 |
| 3279 | CA | PHE | B | 31 | 47.068 | 56.598 | 19.933 | 1.00 | 249.69 |
| 3280 | CB | PHE | B | 31 | 48.220 | 55.917 | 20.703 | 1.00 | 249.69 |
| 3281 | CG | PHE | B | 31 | 49.601 | 56.344 | 20.239 | 1.00 | 249.69 |
| 3282 | CD1 | PHE | B | 31 | 50.151 | 57.556 | 20.662 | 1.00 | 249.69 |
| 3283 | CD2 | PHE | B | 31 | 50.335 | 55.552 | 19.345 | 1.00 | 249.69 |
| 3284 | CE1 | PHE | B | 31 | 51.403 | 57.976 | 20.203 | 1.00 | 249.69 |
| 3285 | CE2 | PHE | B | 31 | 51.589 | 55.967 | 18.881 | 1.00 | 249.69 |
| 3286 | CZ | PHE | B | 31 | 52.121 | 57.182 | 19.313 | 1.00 | 249.69 |
| 3287 | C | PHE | B | 31 | 45.718 | 56.071 | 20.433 | 1.00 | 249.69 |
| 3288 | O | PHE | B | 31 | 45.325 | 56.313 | 21.577 | 1.00 | 249.69 |
| 3289 | N | PHE | B | 32 | 45.027 | 55.341 | 19.556 | 1.00 | 249.69 |
| 3290 | CA | PHE | B | 32 | 43.717 | 54.761 | 19.859 | 1.00 | 249.69 |
| 3291 | CB | PHE | B | 32 | 42.670 | 55.314 | 18.875 | 1.00 | 249.69 |
| 3292 | CG | PHE | B | 32 | 41.238 | 55.081 | 19.304 | 1.00 | 249.69 |
| 3293 | CD1 | PHE | B | 32 | 40.722 | 55.709 | 20.443 | 1.00 | 249.69 |
| 3294 | CD2 | PHE | B | 32 | 40.404 | 54.237 | 18.565 | 1.00 | 249.69 |
| 3295 | CE1 | PHE | B | 32 | 39.398 | 55.499 | 20.835 | 1.00 | 249.69 |
| 3296 | CE2 | PHE | B | 32 | 39.081 | 54.022 | 18.950 | 1.00 | 249.69 |
| 3297 | CZ | PHE | B | 32 | 38.578 | 54.655 | 20.087 | 1.00 | 249.69 |
| 3298 | C | PHE | B | 32 | 43.771 | 53.220 | 19.785 | 1.00 | 249.69 |
| 3299 | O | PHE | B | 32 | 44.746 | 52.646 | 19.285 | 1.00 | 249.69 |
| 3300 | N | GLU | B | 33 | 42.714 | 52.559 | 20.259 | 1.00 | 249.69 |
| 3301 | CA | GLU | B | 33 | 42.688 | 51.104 | 20.273 | 1.00 | 249.69 |
| 3302 | CB | GLU | B | 33 | 42.563 | 50.633 | 21.724 | 1.00 | 249.69 |
| 3303 | CG | GLU | B | 33 | 42.965 | 49.185 | 21.932 | 1.00 | 249.69 |
| 3304 | CD | GLU | B | 33 | 44.299 | 48.858 | 21.285 | 1.00 | 249.69 |
| 3305 | OE1 | GLU | B | 33 | 45.257 | 49.641 | 21.471 | 1.00 | 249.69 |
| 3306 | OE2 | GLU | B | 33 | 44.389 | 47.819 | 20.593 | 1.00 | 249.69 |
| 3307 | C | GLU | B | 33 | 41.644 | 50.384 | 19.409 | 1.00 | 249.69 |
| 3308 | O | GLU | B | 33 | 41.991 | 49.478 | 18.645 | 1.00 | 249.69 |
| 3309 | N | VAL | B | 34 | 40.375 | 50.773 | 19.532 | 1.00 | 249.69 |
| 3310 | CA | VAL | B | 34 | 39.290 | 50.129 | 18.784 | 1.00 | 249.69 |
| 3311 | CB | VAL | B | 34 | 37.920 | 50.767 | 19.158 | 1.00 | 247.84 |
| 3312 | CG1 | VAL | B | 34 | 36.794 | 50.060 | 18.427 | 1.00 | 247.84 |
| 3313 | CG2 | VAL | B | 34 | 37.698 | 50.681 | 20.661 | 1.00 | 247.84 |
| 3314 | C | VAL | B | 34 | 39.448 | 50.119 | 17.252 | 1.00 | 249.69 |
| 3315 | O | VAL | B | 34 | 40.059 | 51.023 | 16.663 | 1.00 | 249.69 |
| 3316 | N | SER | B | 35 | 38.895 | 49.077 | 16.627 | 1.00 | 249.69 |
| 3317 | CA | SER | B | 35 | 38.934 | 48.909 | 15.175 | 1.00 | 249.69 |
| 3318 | CB | SER | B | 35 | 39.389 | 47.500 | 14.806 | 1.00 | 240.73 |
| 3319 | OG | SER | B | 35 | 38.373 | 46.555 | 15.104 | 1.00 | 240.73 |
| 3320 | C | SER | B | 35 | 37.529 | 49.126 | 14.625 | 1.00 | 249.69 |
| 3321 | O | SER | B | 35 | 37.305 | 49.055 | 13.412 | 1.00 | 249.69 |
| 3322 | N | SER | B | 36 | 36.583 | 49.371 | 15.533 | 1.00 | 249.69 |
| 3323 | CA | SER | B | 36 | 35.186 | 49.611 | 15.162 | 1.00 | 249.69 |
| 3324 | CB | SER | B | 36 | 34.233 | 48.825 | 16.081 | 1.00 | 249.69 |
| 3325 | OG | SER | B | 36 | 34.184 | 49.372 | 17.391 | 1.00 | 249.69 |
| 3326 | C | SER | B | 36 | 34.857 | 51.108 | 15.231 | 1.00 | 249.69 |
| 3327 | O | SER | B | 36 | 34.462 | 51.639 | 16.278 | 1.00 | 249.69 |
| 3328 | N | THR | B | 37 | 35.037 | 51.780 | 14.102 | 1.00 | 249.69 |
| 3329 | CA | THR | B | 37 | 34.765 | 53.198 | 14.004 | 1.00 | 249.69 |
| 3330 | CB | THR | B | 37 | 36.076 | 53.989 | 13.773 | 1.00 | 184.28 |
| 3331 | OG1 | THR | B | 37 | 36.977 | 53.759 | 14.866 | 1.00 | 184.28 |
| 3332 | CG2 | THR | B | 37 | 35.787 | 55.477 | 13.679 | 1.00 | 184.28 |
| 3333 | C | THR | B | 37 | 33.808 | 53.375 | 12.823 | 1.00 | 249.69 |
| 3334 | O | THR | B | 37 | 33.964 | 52.726 | 11.782 | 1.00 | 249.69 |
| 3335 | N | LYS | B | 38 | 32.809 | 54.236 | 12.988 | 1.00 | 236.74 |
| 3336 | CA | LYS | B | 38 | 31.831 | 54.464 | 11.930 | 1.00 | 236.74 |
| 3337 | CB | LYS | B | 38 | 30.421 | 54.378 | 12.519 | 1.00 | 245.72 |
| 3338 | CG | LYS | B | 38 | 30.118 | 53.037 | 13.196 | 1.00 | 245.72 |
| 3339 | CD | LYS | B | 38 | 28.713 | 52.984 | 13.800 | 1.00 | 245.72 |
| 3340 | CE | LYS | B | 38 | 28.418 | 51.615 | 14.417 | 1.00 | 245.72 |
| 3341 | NZ | LYS | B | 38 | 27.042 | 51.521 | 14.992 | 1.00 | 245.72 |
| 3342 | C | LYS | B | 38 | 32.035 | 55.806 | 11.227 | 1.00 | 236.74 |
| 3343 | O | LYS | B | 38 | 32.415 | 56.797 | 11.847 | 1.00 | 236.74 |
| 3344 | N | TRP | B | 39 | 31.805 | 55.821 | 9.920 | 1.00 | 197.18 |
| 3345 | CA | TRP | B | 39 | 31.946 | 57.042 | 9.141 | 1.00 | 197.18 |
| 3346 | CB | TRP | B | 39 | 33.131 | 56.937 | 8.184 | 1.00 | 174.88 |
| 3347 | CG | TRP | B | 39 | 34.474 | 56.888 | 8.840 | 1.00 | 174.88 |
| 3348 | CD2 | TRP | B | 39 | 35.068 | 57.877 | 9.709 | 1.00 | 174.88 |
| 3349 | CE2 | TRP | B | 39 | 36.391 | 57.457 | 9.977 | 1.00 | 174.88 |
| 3350 | CE3 | TRP | B | 39 | 34.614 | 59.069 | 10.278 | 1.00 | 174.88 |
| 3351 | CD1 | TRP | B | 39 | 35.433 | 55.942 | 8.634 | 1.00 | 174.88 |
| 3352 | NE1 | TRP | B | 39 | 36.589 | 56.276 | 9.311 | 1.00 | 174.88 |
| 3353 | CZ2 | TRP | B | 39 | 37.265 | 58.190 | 10.788 | 1.00 | 174.88 |
| 3354 | CZ3 | TRP | B | 39 | 35.488 | 59.797 | 11.032 | 1.00 | 174.88 |
| 3355 | CH2 | TRP | B | 39 | 36.798 | 59.357 | 11.324 | 1.00 | 174.88 |
| 3356 | C | TRP | B | 39 | 30.667 | 57.211 | 8.335 | 1.00 | 197.18 |
| 3357 | O | TRP | B | 39 | 30.215 | 56.251 | 7.708 | 1.00 | 197.18 |
| 3358 | N | PHE | B | 40 | 30.086 | 58.413 | 8.348 | 1.00 | 196.05 |
| 3359 | CA | PHE | B | 40 | 28.848 | 58.653 | 7.609 | 1.00 | 196.05 |
| 3360 | CB | PHE | B | 40 | 27.674 | 58.878 | 8.572 | 1.00 | 216.66 |
| 3361 | CG | PHE | B | 40 | 27.425 | 57.738 | 9.528 | 1.00 | 216.66 |
| 3362 | CD1 | PHE | B | 40 | 28.199 | 57.602 | 10.679 | 1.00 | 216.66 |
| 3363 | CD2 | PHE | B | 40 | 26.396 | 56.824 | 9.297 | 1.00 | 216.66 |
| 3364 | CE1 | PHE | B | 40 | 27.951 | 56.578 | 11.590 | 1.00 | 216.66 |
| 3365 | CE2 | PHE | B | 40 | 26.139 | 55.800 | 10.194 | 1.00 | 216.66 |
| 3366 | CZ | PHE | B | 40 | 26.917 | 55.674 | 11.345 | 1.00 | 216.66 |
| 3367 | C | PHE | B | 40 | 28.896 | 59.825 | 6.616 | 1.00 | 196.05 |
| 3368 | O | PHE | B | 40 | 28.336 | 60.888 | 6.877 | 1.00 | 196.05 |
| 3369 | N | HIS | B | 41 | 29.552 | 59.613 | 5.476 | 1.00 | 132.87 |
| 3370 | CA | HIS | B | 41 | 29.665 | 60.618 | 4.418 | 1.00 | 132.87 |
| 3371 | CB | HIS | B | 41 | 30.576 | 60.097 | 3.315 | 1.00 | 148.70 |
| 3372 | CG | HIS | B | 41 | 30.772 | 61.067 | 2.198 | 1.00 | 148.70 |
| 3373 | CD2 | HIS | B | 41 | 30.886 | 60.877 | 0.864 | 1.00 | 148.70 |
| 3374 | ND1 | HIS | B | 41 | 30.949 | 62.416 | 2.414 | 1.00 | 148.70 |
| 3375 | CE1 | HIS | B | 41 | 31.166 | 63.019 | 1.259 | 1.00 | 148.70 |
| 3376 | NE2 | HIS | B | 41 | 31.135 | 62.108 | 0.304 | 1.00 | 148.70 |
| 3377 | C | HIS | B | 41 | 28.311 | 60.975 | 3.804 | 1.00 | 132.87 |
| 3378 | O | HIS | B | 41 | 27.783 | 60.210 | 2.996 | 1.00 | 132.87 |
| 3379 | N | ASN | B | 42 | 27.777 | 62.147 | 4.163 | 1.00 | 209.30 |
| 3380 | CA | ASN | B | 42 | 26.467 | 62.614 | 3.684 | 1.00 | 209.30 |
| 3381 | CB | ASN | B | 42 | 26.371 | 62.546 | 2.148 | 1.00 | 240.31 |
| 3382 | CG | ASN | B | 42 | 27.092 | 63.700 | 1.461 | 1.00 | 240.31 |
| 3383 | OD1 | ASN | B | 42 | 28.241 | 63.989 | 1.789 | 1.00 | 240.31 |
| 3384 | ND2 | ASN | B | 42 | 26.427 | 64.350 | 0.505 | 1.00 | 240.31 |
| 3385 | C | ASN | B | 42 | 25.375 | 61.745 | 4.316 | 1.00 | 209.30 |
| 3386 | O | ASN | B | 42 | 24.271 | 61.613 | 3.774 | 1.00 | 209.30 |
| 3387 | N | GLY | B | 43 | 25.695 | 61.168 | 5.475 | 1.00 | 171.14 |
| 3388 | CA | GLY | B | 43 | 24.758 | 60.306 | 6.176 | 1.00 | 171.14 |
| 3389 | C | GLY | B | 43 | 24.985 | 58.836 | 5.841 | 1.00 | 171.14 |
| 3390 | O | GLY | B | 43 | 24.963 | 57.969 | 6.717 | 1.00 | 171.14 |
| 3391 | N | SER | B | 44 | 25.211 | 58.563 | 4.560 | 1.00 | 214.62 |
| 3392 | CA | SER | B | 44 | 25.444 | 57.207 | 4.071 | 1.00 | 214.62 |
| 3393 | CB | SER | B | 44 | 25.676 | 57.228 | 2.555 | 1.00 | 249.69 |
| 3394 | OG | SER | B | 44 | 24.567 | 57.774 | 1.864 | 1.00 | 249.69 |
| 3395 | C | SER | B | 44 | 26.651 | 56.559 | 4.733 | 1.00 | 214.62 |
| 3396 | O | SER | B | 44 | 27.757 | 57.088 | 4.657 | 1.00 | 214.62 |
| 3397 | N | LEU | B | 45 | 26.448 | 55.404 | 5.359 | 1.00 | 180.32 |
| 3398 | CA | LEU | B | 45 | 27.555 | 54.713 | 6.008 | 1.00 | 180.32 |
| 3399 | CB | LEU | B | 45 | 27.095 | 53.361 | 6.558 | 1.00 | 218.81 |
| 3400 | CG | LEU | B | 45 | 28.170 | 52.573 | 7.318 | 1.00 | 218.81 |
| 3401 | CD1 | LEU | B | 45 | 28.753 | 53.429 | 8.432 | 1.00 | 218.81 |
| 3402 | CD2 | LEU | B | 45 | 27.568 | 51.300 | 7.883 | 1.00 | 218.81 |
| 3403 | C | LEU | B | 45 | 28.722 | 54.516 | 5.030 | 1.00 | 180.32 |
| 3404 | O | LEU | B | 45 | 28.517 | 54.379 | 3.819 | 1.00 | 180.32 |
| 3405 | N | SER | B | 46 | 29.942 | 54.517 | 5.565 | 1.00 | 248.98 |
| 3406 | CA | SER | B | 46 | 31.145 | 54.357 | 4.757 | 1.00 | 248.98 |
| 3407 | CB | SER | B | 46 | 32.188 | 55.405 | 5.149 | 1.00 | 249.38 |
| 3408 | OG | SER | B | 46 | 33.322 | 55.338 | 4.301 | 1.00 | 249.38 |
| 3409 | C | SER | B | 46 | 31.747 | 52.967 | 4.898 | 1.00 | 248.98 |
| 3410 | O | SER | B | 46 | 31.351 | 52.195 | 5.773 | 1.00 | 248.98 |
| 3411 | N | GLU | B | 47 | 32.727 | 52.670 | 4.045 | 1.00 | 249.69 |
| 3412 | CA | GLU | B | 47 | 33.387 | 51.365 | 4.032 | 1.00 | 249.69 |
| 3413 | CB | GLU | B | 47 | 33.757 | 50.996 | 2.593 | 1.00 | 249.69 |
| 3414 | CG | GLU | B | 47 | 32.553 | 50.869 | 1.674 | 1.00 | 249.69 |
| 3415 | CD | GLU | B | 47 | 32.945 | 50.524 | 0.256 | 1.00 | 249.69 |
| 3416 | OE1 | GLU | B | 47 | 33.631 | 51.350 | −0.388 | 1.00 | 249.69 |
| 3417 | OE2 | GLU | B | 47 | 32.572 | 49.426 | −0.214 | 1.00 | 249.69 |
| 3418 | C | GLU | B | 47 | 34.618 | 51.211 | 4.926 | 1.00 | 249.69 |
| 3419 | O | GLU | B | 47 | 35.099 | 50.096 | 5.132 | 1.00 | 249.69 |
| 3420 | N | GLU | B | 48 | 35.139 | 52.316 | 5.446 | 1.00 | 184.88 |
| 3421 | CA | GLU | B | 48 | 36.297 | 52.225 | 6.316 | 1.00 | 184.88 |
| 3422 | CB | GLU | B | 48 | 37.110 | 53.523 | 6.288 | 1.00 | 208.21 |
| 3423 | CG | GLU | B | 48 | 38.293 | 53.542 | 7.265 | 1.00 | 208.21 |
| 3424 | CD | GLU | B | 48 | 39.366 | 52.513 | 6.941 | 1.00 | 208.21 |
| 3425 | OE1 | GLU | B | 48 | 40.084 | 52.698 | 5.937 | 1.00 | 208.21 |
| 3426 | OE2 | GLU | B | 48 | 39.493 | 51.517 | 7.685 | 1.00 | 208.21 |
| 3427 | C | GLU | B | 48 | 35.834 | 51.929 | 7.738 | 1.00 | 184.88 |
| 3428 | O | GLU | B | 48 | 34.687 | 52.213 | 8.104 | 1.00 | 184.88 |
| 3429 | N | THR | B | 49 | 36.731 | 51.348 | 8.530 | 1.00 | 237.20 |
| 3430 | CA | THR | B | 49 | 36.443 | 51.003 | 9.917 | 1.00 | 237.20 |
| 3431 | CB | THR | B | 49 | 36.348 | 49.477 | 10.090 | 1.00 | 231.33 |
| 3432 | OG1 | THR | B | 49 | 37.542 | 48.864 | 9.586 | 1.00 | 231.33 |
| 3433 | CG2 | THR | B | 49 | 35.144 | 48.933 | 9.336 | 1.00 | 231.33 |
| 3434 | C | THR | B | 49 | 37.540 | 51.551 | 10.829 | 1.00 | 237.20 |
| 3435 | O | THR | B | 49 | 37.303 | 51.819 | 12.006 | 1.00 | 237.20 |
| 3436 | N | ASN | B | 50 | 38.739 | 51.713 | 10.278 | 1.00 | 222.02 |
| 3437 | CA | ASN | B | 50 | 39.863 | 52.246 | 11.036 | 1.00 | 222.02 |
| 3438 | CB | ASN | B | 50 | 41.101 | 52.350 | 10.132 | 1.00 | 245.81 |
| 3439 | CG | ASN | B | 50 | 42.369 | 52.650 | 10.910 | 1.00 | 245.81 |
| 3440 | OD1 | ASN | B | 50 | 42.309 | 53.119 | 12.045 | 1.00 | 245.81 |
| 3441 | ND2 | ASN | B | 50 | 43.520 | 52.400 | 10.299 | 1.00 | 245.81 |
| 3442 | C | ASN | B | 50 | 39.447 | 53.636 | 11.531 | 1.00 | 222.02 |
| 3443 | O | ASN | B | 50 | 38.625 | 54.290 | 10.901 | 1.00 | 222.02 |
| 3444 | N | SER | B | 51 | 40.004 | 54.089 | 12.651 | 1.00 | 208.27 |
| 3445 | CA | SER | B | 51 | 39.652 | 55.405 | 13.176 | 1.00 | 208.27 |
| 3446 | CB | SER | B | 51 | 40.219 | 55.590 | 14.590 | 1.00 | 249.69 |
| 3447 | OG | SER | B | 51 | 41.624 | 55.785 | 14.565 | 1.00 | 249.69 |
| 3448 | C | SER | B | 51 | 40.135 | 56.555 | 12.276 | 1.00 | 208.27 |
| 3449 | O | SER | B | 51 | 39.672 | 57.690 | 12.416 | 1.00 | 208.27 |
| 3450 | N | SER | B | 52 | 41.059 | 56.265 | 11.360 | 1.00 | 249.32 |
| 3451 | CA | SER | B | 52 | 41.588 | 57.283 | 10.446 | 1.00 | 249.32 |
| 3452 | CB | SER | B | 52 | 43.125 | 57.283 | 10.456 | 1.00 | 193.43 |
| 3453 | OG | SER | B | 52 | 43.642 | 57.584 | 11.741 | 1.00 | 193.43 |
| 3454 | C | SER | B | 52 | 41.106 | 57.076 | 9.014 | 1.00 | 249.32 |
| 3455 | O | SER | B | 52 | 41.596 | 56.199 | 8.299 | 1.00 | 249.32 |
| 3456 | N | LEU | B | 53 | 40.147 | 57.895 | 8.601 | 1.00 | 187.33 |
| 3457 | CA | LEU | B | 53 | 39.601 | 57.820 | 7.255 | 1.00 | 187.33 |
| 3458 | CB | LEU | B | 53 | 38.107 | 58.131 | 7.283 | 1.00 | 113.92 |
| 3459 | CG | LEU | B | 53 | 37.410 | 58.539 | 5.975 | 1.00 | 113.92 |
| 3460 | CD1 | LEU | B | 53 | 37.839 | 57.637 | 4.810 | 1.00 | 113.92 |
| 3461 | CD2 | LEU | B | 53 | 35.889 | 58.503 | 6.185 | 1.00 | 113.92 |
| 3462 | C | LEU | B | 53 | 40.310 | 58.794 | 6.331 | 1.00 | 187.33 |
| 3463 | O | LEU | B | 53 | 40.085 | 60.010 | 6.397 | 1.00 | 187.33 |
| 3464 | N | ASN | B | 54 | 41.169 | 58.261 | 5.467 | 1.00 | 190.27 |
| 3465 | CA | ASN | B | 54 | 41.899 | 59.112 | 4.547 | 1.00 | 190.27 |
| 3466 | CB | ASN | B | 54 | 43.209 | 58.458 | 4.126 | 1.00 | 248.26 |
| 3467 | CG | ASN | B | 54 | 44.214 | 58.414 | 5.254 | 1.00 | 248.26 |
| 3468 | OD1 | ASN | B | 54 | 44.519 | 59.432 | 5.876 | 1.00 | 248.26 |
| 3469 | ND2 | ASN | B | 54 | 44.737 | 57.232 | 5.525 | 1.00 | 248.26 |
| 3470 | C | ASN | B | 54 | 41.096 | 59.481 | 3.320 | 1.00 | 190.27 |
| 3471 | O | ASN | B | 54 | 40.134 | 58.800 | 2.957 | 1.00 | 190.27 |
| 3472 | N | ILE | B | 55 | 41.515 | 60.580 | 2.700 | 1.00 | 195.13 |
| 3473 | CA | ILE | B | 55 | 40.900 | 61.127 | 1.503 | 1.00 | 195.13 |
| 3474 | CB | ILE | B | 55 | 40.101 | 62.413 | 1.829 | 1.00 | 126.27 |
| 3475 | CG2 | ILE | B | 55 | 39.946 | 63.268 | 0.581 | 1.00 | 126.27 |
| 3476 | CG1 | ILE | B | 55 | 38.743 | 62.032 | 2.431 | 1.00 | 126.27 |
| 3477 | CD1 | ILE | B | 55 | 37.857 | 63.206 | 2.786 | 1.00 | 126.27 |
| 3478 | C | ILE | B | 55 | 42.017 | 61.473 | 0.540 | 1.00 | 195.13 |
| 3479 | O | ILE | B | 55 | 42.836 | 62.346 | 0.825 | 1.00 | 195.13 |
| 3480 | N | VAL | B | 56 | 42.057 | 60.789 | −0.594 | 1.00 | 178.85 |
| 3481 | CA | VAL | B | 56 | 43.099 | 61.058 | −1.569 | 1.00 | 178.85 |
| 3482 | CB | VAL | B | 56 | 43.587 | 59.773 | −2.227 | 1.00 | 249.69 |
| 3483 | CG1 | VAL | B | 56 | 44.960 | 60.002 | −2.841 | 1.00 | 249.69 |
| 3484 | CG2 | VAL | B | 56 | 43.642 | 58.662 | −1.189 | 1.00 | 249.69 |
| 3485 | C | VAL | B | 56 | 42.580 | 62.012 | −2.630 | 1.00 | 178.85 |
| 3486 | O | VAL | B | 56 | 41.612 | 62.729 | −2.376 | 1.00 | 178.85 |
| 3487 | N | ASN | B | 57 | 43.217 | 62.025 | −3.804 | 1.00 | 192.84 |
| 3488 | CA | ASN | B | 57 | 42.832 | 62.923 | −4.895 | 1.00 | 192.84 |
| 3489 | CB | ASN | B | 57 | 43.085 | 62.261 | −6.239 | 1.00 | 201.25 |
| 3490 | CG | ASN | B | 57 | 44.560 | 62.119 | −6.530 | 1.00 | 201.25 |
| 3491 | OD1 | ASN | B | 57 | 45.309 | 63.090 | −6.446 | 1.00 | 201.25 |
| 3492 | ND2 | ASN | B | 57 | 44.988 | 60.908 | −6.875 | 1.00 | 201.25 |
| 3493 | C | ASN | B | 57 | 41.397 | 63.405 | −4.791 | 1.00 | 192.84 |
| 3494 | O | ASN | B | 57 | 40.470 | 62.757 | −5.267 | 1.00 | 192.84 |
| 3495 | N | ALA | B | 58 | 41.253 | 64.565 | −4.151 | 1.00 | 127.65 |
| 3496 | CA | ALA | B | 58 | 39.980 | 65.227 | −3.870 | 1.00 | 127.65 |
| 3497 | CB | ALA | B | 58 | 40.256 | 66.555 | −3.201 | 1.00 | 133.74 |
| 3498 | C | ALA | B | 58 | 39.029 | 65.436 | −5.041 | 1.00 | 127.65 |
| 3499 | O | ALA | B | 58 | 39.244 | 66.294 | −5.891 | 1.00 | 127.65 |
| 3500 | N | LYS | B | 59 | 37.956 | 64.658 | −5.060 | 1.00 | 124.63 |
| 3501 | CA | LYS | B | 59 | 36.946 | 64.744 | −6.109 | 1.00 | 124.63 |
| 3502 | CB | LYS | B | 59 | 36.504 | 63.344 | −6.550 | 1.00 | 240.20 |
| 3503 | CG | LYS | B | 59 | 37.632 | 62.491 | −7.108 | 1.00 | 240.20 |
| 3504 | CD | LYS | B | 59 | 37.182 | 61.069 | −7.421 | 1.00 | 240.20 |
| 3505 | CE | LYS | B | 59 | 38.354 | 60.214 | −7.901 | 1.00 | 240.20 |
| 3506 | NZ | LYS | B | 59 | 37.959 | 58.816 | −8.237 | 1.00 | 240.20 |
| 3507 | C | LYS | B | 59 | 35.778 | 65.493 | −5.511 | 1.00 | 124.63 |
| 3508 | O | LYS | B | 59 | 35.423 | 65.278 | −4.355 | 1.00 | 124.63 |
| 3509 | N | PHE | B | 60 | 35.188 | 66.378 | −6.301 | 1.00 | 146.51 |
| 3510 | CA | PHE | B | 60 | 34.064 | 67.179 | −5.839 | 1.00 | 146.51 |
| 3511 | CB | PHE | B | 60 | 33.350 | 67.788 | −7.049 | 1.00 | 206.05 |
| 3512 | CG | PHE | B | 60 | 34.189 | 68.770 | −7.815 | 1.00 | 206.05 |
| 3513 | CD1 | PHE | B | 60 | 33.968 | 68.976 | −9.169 | 1.00 | 206.05 |
| 3514 | CD2 | PHE | B | 60 | 35.193 | 69.499 | −7.177 | 1.00 | 206.05 |
| 3515 | CE1 | PHE | B | 60 | 34.732 | 69.890 | −9.882 | 1.00 | 206.05 |
| 3516 | CE2 | PHE | B | 60 | 35.963 | 70.417 | −7.877 | 1.00 | 206.05 |
| 3517 | CZ | PHE | B | 60 | 35.732 | 70.612 | −9.234 | 1.00 | 206.05 |
| 3518 | C | PHE | B | 60 | 33.073 | 66.407 | −4.968 | 1.00 | 146.51 |
| 3519 | O | PHE | B | 60 | 32.479 | 66.957 | −4.044 | 1.00 | 146.51 |
| 3520 | N | GLU | B | 61 | 32.911 | 65.125 | −5.257 | 1.00 | 158.05 |
| 3521 | CA | GLU | B | 61 | 31.977 | 64.284 | −4.520 | 1.00 | 158.05 |
| 3522 | CB | GLU | B | 61 | 31.872 | 62.911 | −5.197 | 1.00 | 249.69 |
| 3523 | CG | GLU | B | 61 | 31.394 | 62.950 | −6.651 | 1.00 | 249.69 |
| 3524 | CD | GLU | B | 61 | 32.288 | 63.798 | −7.556 | 1.00 | 249.69 |
| 3525 | OE1 | GLU | B | 61 | 33.522 | 63.580 | −7.574 | 1.00 | 249.69 |
| 3526 | OE2 | GLU | B | 61 | 31.753 | 64.685 | −8.256 | 1.00 | 249.69 |
| 3527 | C | GLU | B | 61 | 32.391 | 64.113 | −3.067 | 1.00 | 158.05 |
| 3528 | O | GLU | B | 61 | 31.555 | 63.831 | −2.213 | 1.00 | 158.05 |
| 3529 | N | ASP | B | 62 | 33.682 | 64.285 | −2.789 | 1.00 | 134.92 |
| 3530 | CA | ASP | B | 62 | 34.200 | 64.139 | −1.426 | 1.00 | 134.92 |
| 3531 | CB | ASP | B | 62 | 35.729 | 64.128 | −1.432 | 1.00 | 133.92 |
| 3532 | CG | ASP | B | 62 | 36.294 | 63.118 | −2.393 | 1.00 | 133.92 |
| 3533 | OD1 | ASP | B | 62 | 35.669 | 62.048 | −2.551 | 1.00 | 133.92 |
| 3534 | OD2 | ASP | B | 62 | 37.364 | 63.397 | −2.972 | 1.00 | 133.92 |
| 3535 | C | ASP | B | 62 | 33.709 | 65.279 | −0.543 | 1.00 | 134.92 |
| 3536 | O | ASP | B | 62 | 33.663 | 65.143 | 0.685 | 1.00 | 134.92 |
| 3537 | N | SER | B | 63 | 33.358 | 66.400 | −1.177 | 1.00 | 128.21 |
| 3538 | CA | SER | B | 63 | 32.857 | 67.553 | −0.449 | 1.00 | 128.21 |
| 3539 | CB | SER | B | 63 | 32.552 | 68.710 | −1.407 | 1.00 | 212.43 |
| 3540 | OG | SER | B | 63 | 33.696 | 69.095 | −2.153 | 1.00 | 212.43 |
| 3541 | C | SER | B | 63 | 31.574 | 67.137 | 0.261 | 1.00 | 128.21 |
| 3542 | O | SER | B | 63 | 30.660 | 66.629 | −0.377 | 1.00 | 128.21 |
| 3543 | N | GLY | B | 64 | 31.500 | 67.347 | 1.571 | 1.00 | 137.99 |
| 3544 | CA | GLY | B | 64 | 30.298 | 66.963 | 2.274 | 1.00 | 137.99 |
| 3545 | C | GLY | B | 64 | 30.367 | 66.973 | 3.784 | 1.00 | 137.99 |
| 3546 | O | GLY | B | 64 | 31.282 | 67.545 | 4.385 | 1.00 | 137.99 |
| 3547 | N | GLU | B | 65 | 29.378 | 66.312 | 4.383 | 1.00 | 125.38 |
| 3548 | CA | GLU | B | 65 | 29.219 | 66.217 | 5.830 | 1.00 | 125.38 |
| 3549 | CB | GLU | B | 65 | 27.747 | 66.504 | 6.159 | 1.00 | 249.57 |
| 3550 | CG | GLU | B | 65 | 27.329 | 66.290 | 7.594 | 1.00 | 249.57 |
| 3551 | CD | GLU | B | 65 | 25.818 | 66.198 | 7.731 | 1.00 | 249.57 |
| 3552 | OE1 | GLU | B | 65 | 25.227 | 65.270 | 7.143 | 1.00 | 249.57 |
| 3553 | OE2 | GLU | B | 65 | 25.217 | 67.046 | 8.416 | 1.00 | 249.57 |
| 3554 | C | GLU | B | 65 | 29.617 | 64.843 | 6.339 | 1.00 | 125.38 |
| 3555 | O | GLU | B | 65 | 29.050 | 63.844 | 5.900 | 1.00 | 125.38 |
| 3556 | N | TYR | B | 66 | 30.575 | 64.790 | 7.265 | 1.00 | 138.71 |
| 3557 | CA | TYR | B | 66 | 31.029 | 63.510 | 7.826 | 1.00 | 138.71 |
| 3558 | CB | TYR | B | 66 | 32.512 | 63.294 | 7.560 | 1.00 | 186.43 |
| 3559 | CG | TYR | B | 66 | 32.917 | 63.170 | 6.122 | 1.00 | 186.43 |
| 3560 | CD1 | TYR | B | 66 | 32.997 | 64.287 | 5.302 | 1.00 | 186.43 |
| 3561 | CE1 | TYR | B | 66 | 33.473 | 64.189 | 3.998 | 1.00 | 186.43 |
| 3562 | CD2 | TYR | B | 66 | 33.309 | 61.943 | 5.607 | 1.00 | 186.43 |
| 3563 | CE2 | TYR | B | 66 | 33.785 | 61.827 | 4.314 | 1.00 | 186.43 |
| 3564 | CZ | TYR | B | 66 | 33.871 | 62.957 | 3.508 | 1.00 | 186.43 |
| 3565 | OH | TYR | B | 66 | 34.375 | 62.850 | 2.220 | 1.00 | 186.43 |
| 3566 | C | TYR | B | 66 | 30.823 | 63.378 | 9.345 | 1.00 | 138.71 |
| 3567 | O | TYR | B | 66 | 30.510 | 64.370 | 10.022 | 1.00 | 138.71 |
| 3568 | N | LYS | B | 67 | 31.029 | 62.155 | 9.862 | 1.00 | 179.70 |
| 3569 | CA | LYS | B | 67 | 30.895 | 61.847 | 11.295 | 1.00 | 179.70 |
| 3570 | CB | LYS | B | 67 | 29.456 | 62.047 | 11.754 | 1.00 | 159.69 |
| 3571 | CG | LYS | B | 67 | 28.447 | 61.317 | 10.919 | 1.00 | 159.69 |
| 3572 | CD | LYS | B | 67 | 27.057 | 61.654 | 11.392 | 1.00 | 159.69 |
| 3573 | CE | LYS | B | 67 | 26.005 | 61.334 | 10.324 | 1.00 | 159.69 |
| 3574 | NZ | LYS | B | 67 | 24.591 | 61.613 | 10.773 | 1.00 | 159.69 |
| 3575 | C | LYS | B | 67 | 31.323 | 60.434 | 11.691 | 1.00 | 179.70 |
| 3576 | O | LYS | B | 67 | 31.284 | 59.505 | 10.885 | 1.00 | 179.70 |
| 3577 | N | CYS | B | 68 | 31.723 | 60.285 | 12.953 | 1.00 | 162.03 |
| 3578 | CA | CYS | B | 68 | 32.142 | 58.993 | 13.489 | 1.00 | 162.03 |
| 3579 | C | CYS | B | 68 | 31.445 | 58.691 | 14.810 | 1.00 | 162.03 |
| 3580 | O | CYS | B | 68 | 31.102 | 59.589 | 15.578 | 1.00 | 162.03 |
| 3581 | CB | CYS | B | 68 | 33.670 | 58.915 | 13.665 | 1.00 | 220.63 |
| 3582 | SG | CYS | B | 68 | 34.426 | 59.969 | 14.951 | 1.00 | 220.63 |
| 3583 | N | GLN | B | 69 | 31.239 | 57.402 | 15.049 | 1.00 | 249.69 |
| 3584 | CA | GLN | B | 69 | 30.573 | 56.906 | 16.245 | 1.00 | 249.69 |
| 3585 | CB | GLN | B | 69 | 29.078 | 56.763 | 15.965 | 1.00 | 222.67 |
| 3586 | CG | GLN | B | 69 | 28.343 | 55.832 | 16.904 | 1.00 | 222.67 |
| 3587 | CD | GLN | B | 69 | 26.898 | 55.619 | 16.496 | 1.00 | 222.67 |
| 3588 | OE1 | GLN | B | 69 | 26.612 | 55.268 | 15.351 | 1.00 | 222.67 |
| 3589 | NE2 | GLN | B | 69 | 25.977 | 55.823 | 17.435 | 1.00 | 222.67 |
| 3590 | C | GLN | B | 69 | 31.168 | 55.546 | 16.606 | 1.00 | 249.69 |
| 3591 | O | GLN | B | 69 | 31.580 | 54.788 | 15.727 | 1.00 | 249.69 |
| 3592 | N | HIS | B | 70 | 31.219 | 55.235 | 17.897 | 1.00 | 249.63 |
| 3593 | CA | HIS | B | 70 | 31.762 | 53.956 | 18.331 | 1.00 | 249.63 |
| 3594 | CB | HIS | B | 70 | 32.637 | 54.139 | 19.570 | 1.00 | 248.69 |
| 3595 | CG | HIS | B | 70 | 33.932 | 54.841 | 19.289 | 1.00 | 248.69 |
| 3596 | CD2 | HIS | B | 70 | 34.500 | 55.925 | 19.864 | 1.00 | 248.69 |
| 3597 | ND1 | HIS | B | 70 | 34.798 | 54.422 | 18.304 | 1.00 | 248.69 |
| 3598 | CE1 | HIS | B | 70 | 35.852 | 55.223 | 18.283 | 1.00 | 248.69 |
| 3599 | NE2 | HIS | B | 70 | 35.696 | 56.141 | 19.217 | 1.00 | 248.69 |
| 3600 | C | HIS | B | 70 | 30.656 | 52.952 | 18.618 | 1.00 | 249.63 |
| 3601 | O | HIS | B | 70 | 29.488 | 53.196 | 18.308 | 1.00 | 249.63 |
| 3602 | N | GLN | B | 71 | 31.032 | 51.824 | 19.211 | 1.00 | 249.69 |
| 3603 | CA | GLN | B | 71 | 30.087 | 50.755 | 19.541 | 1.00 | 249.69 |
| 3604 | CB | GLN | B | 71 | 30.836 | 49.613 | 20.251 | 1.00 | 249.69 |
| 3605 | CG | GLN | B | 71 | 30.136 | 48.247 | 20.254 | 1.00 | 249.69 |
| 3606 | CD | GLN | B | 71 | 29.921 | 47.672 | 18.850 | 1.00 | 249.69 |
| 3607 | OE1 | GLN | B | 71 | 30.834 | 47.657 | 18.016 | 1.00 | 249.69 |
| 3608 | NE2 | GLN | B | 71 | 28.710 | 47.183 | 18.594 | 1.00 | 249.69 |
| 3609 | C | GLN | B | 71 | 28.923 | 51.253 | 20.410 | 1.00 | 249.69 |
| 3610 | O | GLN | B | 71 | 27.756 | 50.990 | 20.118 | 1.00 | 249.69 |
| 3611 | N | GLN | B | 72 | 29.243 | 51.983 | 21.471 | 1.00 | 249.69 |
| 3612 | CA | GLN | B | 72 | 28.217 | 52.506 | 22.371 | 1.00 | 249.69 |
| 3613 | CB | GLN | B | 72 | 28.211 | 51.678 | 23.671 | 1.00 | 249.69 |
| 3614 | CG | GLN | B | 72 | 27.259 | 52.118 | 24.772 | 1.00 | 249.69 |
| 3615 | CD | GLN | B | 72 | 27.523 | 51.390 | 26.084 | 1.00 | 249.69 |
| 3616 | OE1 | GLN | B | 72 | 27.540 | 50.159 | 26.130 | 1.00 | 249.69 |
| 3617 | NE2 | GLN | B | 72 | 27.729 | 52.151 | 27.156 | 1.00 | 249.69 |
| 3618 | C | GLN | B | 72 | 28.463 | 53.997 | 22.656 | 1.00 | 249.69 |
| 3619 | O | GLN | B | 72 | 28.598 | 54.423 | 23.809 | 1.00 | 249.69 |
| 3620 | N | VAL | B | 73 | 28.533 | 54.787 | 21.588 | 1.00 | 249.69 |
| 3621 | CA | VAL | B | 73 | 28.747 | 56.226 | 21.718 | 1.00 | 249.69 |
| 3622 | CB | VAL | B | 73 | 30.224 | 56.626 | 21.458 | 1.00 | 227.88 |
| 3623 | CG1 | VAL | B | 73 | 30.454 | 58.055 | 21.917 | 1.00 | 227.88 |
| 3624 | CG2 | VAL | B | 73 | 31.167 | 55.684 | 22.175 | 1.00 | 227.88 |
| 3625 | C | VAL | B | 73 | 27.875 | 56.941 | 20.690 | 1.00 | 249.69 |
| 3626 | O | VAL | B | 73 | 27.681 | 56.449 | 19.579 | 1.00 | 249.69 |
| 3627 | N | ASN | B | 74 | 27.348 | 58.101 | 21.062 | 1.00 | 249.69 |
| 3628 | CA | ASN | B | 74 | 26.504 | 58.866 | 20.156 | 1.00 | 249.69 |
| 3629 | CB | ASN | B | 74 | 25.675 | 59.883 | 20.956 | 1.00 | 218.87 |
| 3630 | CG | ASN | B | 74 | 24.852 | 59.227 | 22.059 | 1.00 | 218.87 |
| 3631 | OD1 | ASN | B | 74 | 24.270 | 58.159 | 21.845 | 1.00 | 218.87 |
| 3632 | ND2 | ASN | B | 74 | 24.793 | 59.865 | 23.226 | 1.00 | 218.87 |
| 3633 | C | ASN | B | 74 | 27.368 | 59.564 | 19.100 | 1.00 | 249.69 |
| 3634 | O | ASN | B | 74 | 28.342 | 60.240 | 19.428 | 1.00 | 249.69 |
| 3635 | N | GLU | B | 75 | 27.005 | 59.383 | 17.833 | 1.00 | 242.43 |
| 3636 | CA | GLU | B | 75 | 27.735 | 59.968 | 16.704 | 1.00 | 242.43 |
| 3637 | CB | GLU | B | 75 | 26.889 | 59.853 | 15.429 | 1.00 | 240.14 |
| 3638 | CG | GLU | B | 75 | 25.394 | 60.044 | 15.649 | 1.00 | 240.14 |
| 3639 | CD | GLU | B | 75 | 24.571 | 59.681 | 14.423 | 1.00 | 240.14 |
| 3640 | OE1 | GLU | B | 75 | 24.782 | 58.580 | 13.867 | 1.00 | 240.14 |
| 3641 | OE2 | GLU | B | 75 | 23.709 | 60.493 | 14.021 | 1.00 | 240.14 |
| 3642 | C | GLU | B | 75 | 28.199 | 61.410 | 16.900 | 1.00 | 242.43 |
| 3643 | O | GLU | B | 75 | 27.478 | 62.241 | 17.453 | 1.00 | 242.43 |
| 3644 | N | SER | B | 76 | 29.413 | 61.690 | 16.427 | 1.00 | 249.69 |
| 3645 | CA | SER | B | 76 | 30.030 | 63.012 | 16.552 | 1.00 | 249.69 |
| 3646 | CB | SER | B | 76 | 31.491 | 62.960 | 16.091 | 1.00 | 193.15 |
| 3647 | OG | SER | B | 76 | 31.574 | 62.865 | 14.681 | 1.00 | 193.15 |
| 3648 | C | SER | B | 76 | 29.323 | 64.104 | 15.766 | 1.00 | 249.69 |
| 3649 | O | SER | B | 76 | 28.595 | 63.825 | 14.813 | 1.00 | 249.69 |
| 3650 | N | GLU | B | 77 | 29.555 | 65.351 | 16.175 | 1.00 | 227.37 |
| 3651 | CA | GLU | B | 77 | 28.969 | 66.496 | 15.500 | 1.00 | 227.37 |
| 3652 | CB | GLU | B | 77 | 29.300 | 67.790 | 16.252 | 1.00 | 249.69 |
| 3653 | CG | GLU | B | 77 | 28.667 | 67.874 | 17.635 | 1.00 | 249.69 |
| 3654 | CD | GLU | B | 77 | 27.144 | 67.885 | 17.588 | 1.00 | 249.69 |
| 3655 | OE1 | GLU | B | 77 | 26.566 | 67.679 | 16.493 | 1.00 | 249.69 |
| 3656 | OE2 | GLU | B | 77 | 26.521 | 68.094 | 18.654 | 1.00 | 249.69 |
| 3657 | C | GLU | B | 77 | 29.556 | 66.554 | 14.099 | 1.00 | 227.37 |
| 3658 | O | GLU | B | 77 | 30.747 | 66.802 | 13.926 | 1.00 | 227.37 |
| 3659 | N | PRO | B | 78 | 28.708 | 66.324 | 13.072 | 1.00 | 151.66 |
| 3660 | CD | PRO | B | 78 | 27.233 | 66.271 | 13.163 | 1.00 | 139.99 |
| 3661 | CA | PRO | B | 78 | 29.168 | 66.339 | 11.671 | 1.00 | 151.66 |
| 3662 | CB | PRO | B | 78 | 27.852 | 66.486 | 10.892 | 1.00 | 139.99 |
| 3663 | CG | PRO | B | 78 | 26.833 | 65.808 | 11.784 | 1.00 | 139.99 |
| 3664 | C | PRO | B | 78 | 30.136 | 67.473 | 11.369 | 1.00 | 151.66 |
| 3665 | O | PRO | B | 78 | 30.182 | 68.466 | 12.086 | 1.00 | 151.66 |
| 3666 | N | VAL | B | 79 | 30.929 | 67.306 | 10.321 | 1.00 | 174.84 |
| 3667 | CA | VAL | B | 79 | 31.855 | 68.352 | 9.905 | 1.00 | 174.84 |
| 3668 | CB | VAL | B | 79 | 33.336 | 68.021 | 10.222 | 1.00 | 152.46 |
| 3669 | CG1 | VAL | B | 79 | 34.256 | 69.029 | 9.536 | 1.00 | 152.46 |
| 3670 | CG2 | VAL | B | 79 | 33.566 | 68.076 | 11.714 | 1.00 | 152.46 |
| 3671 | C | VAL | B | 79 | 31.682 | 68.435 | 8.405 | 1.00 | 174.84 |
| 3672 | O | VAL | B | 79 | 31.482 | 67.408 | 7.752 | 1.00 | 174.84 |
| 3673 | N | TYR | B | 80 | 31.738 | 69.642 | 7.850 | 1.00 | 124.58 |
| 3674 | CA | TYR | B | 80 | 31.564 | 69.771 | 6.414 | 1.00 | 124.58 |
| 3675 | CB | TYR | B | 80 | 30.573 | 70.877 | 6.084 | 1.00 | 201.47 |
| 3676 | CG | TYR | B | 80 | 30.044 | 70.777 | 4.675 | 1.00 | 201.47 |
| 3677 | CD1 | TYR | B | 80 | 28.979 | 69.936 | 4.370 | 1.00 | 201.47 |
| 3678 | CE1 | TYR | B | 80 | 28.515 | 69.801 | 3.072 | 1.00 | 201.47 |
| 3679 | CD2 | TYR | B | 80 | 30.636 | 71.484 | 3.641 | 1.00 | 201.47 |
| 3680 | CE2 | TYR | B | 80 | 30.180 | 71.356 | 2.334 | 1.00 | 201.47 |
| 3681 | CZ | TYR | B | 80 | 29.122 | 70.514 | 2.057 | 1.00 | 201.47 |
| 3682 | OH | TYR | B | 80 | 28.676 | 70.383 | 0.762 | 1.00 | 201.47 |
| 3683 | C | TYR | B | 80 | 32.861 | 70.049 | 5.704 | 1.00 | 124.58 |
| 3684 | O | TYR | B | 80 | 33.655 | 70.887 | 6.140 | 1.00 | 124.58 |
| 3685 | N | LEU | B | 81 | 33.082 | 69.344 | 4.606 | 1.00 | 114.91 |
| 3686 | CA | LEU | B | 81 | 34.296 | 69.544 | 3.839 | 1.00 | 114.91 |
| 3687 | CB | LEU | B | 81 | 35.033 | 68.223 | 3.661 | 1.00 | 104.59 |
| 3688 | CG | LEU | B | 81 | 36.234 | 68.327 | 2.720 | 1.00 | 104.59 |
| 3689 | CD1 | LEU | B | 81 | 37.234 | 69.306 | 3.327 | 1.00 | 104.59 |
| 3690 | CD2 | LEU | B | 81 | 36.881 | 66.972 | 2.487 | 1.00 | 104.59 |
| 3691 | C | LEU | B | 81 | 33.917 | 70.087 | 2.482 | 1.00 | 114.91 |
| 3692 | O | LEU | B | 81 | 33.039 | 69.517 | 1.836 | 1.00 | 114.91 |
| 3693 | N | GLU | B | 82 | 34.545 | 71.184 | 2.049 | 1.00 | 120.70 |
| 3694 | CA | GLU | B | 82 | 34.244 | 71.734 | 0.725 | 1.00 | 120.70 |
| 3695 | CB | GLU | B | 82 | 33.716 | 73.163 | 0.833 | 1.00 | 201.66 |
| 3696 | CG | GLU | B | 82 | 32.820 | 73.551 | −0.332 | 1.00 | 201.66 |
| 3697 | CD | GLU | B | 82 | 32.280 | 74.954 | −0.214 | 1.00 | 201.66 |
| 3698 | OE1 | GLU | B | 82 | 32.022 | 75.401 | 0.925 | 1.00 | 201.66 |
| 3699 | OE2 | GLU | B | 82 | 32.100 | 75.610 | −1.264 | 1.00 | 201.66 |
| 3700 | C | GLU | B | 82 | 35.482 | 71.691 | −0.174 | 1.00 | 120.70 |
| 3701 | O | GLU | B | 82 | 36.583 | 72.004 | 0.276 | 1.00 | 120.70 |
| 3702 | N | VAL | B | 83 | 35.299 | 71.276 | −1.430 | 1.00 | 157.10 |
| 3703 | CA | VAL | B | 83 | 36.398 | 71.204 | −2.390 | 1.00 | 157.10 |
| 3704 | CB | VAL | B | 83 | 36.519 | 69.829 | −3.035 | 1.00 | 142.28 |
| 3705 | CG1 | VAL | B | 83 | 37.699 | 69.820 | −4.005 | 1.00 | 142.28 |
| 3706 | CG2 | VAL | B | 83 | 36.708 | 68.770 | −1.955 | 1.00 | 142.28 |
| 3707 | C | VAL | B | 83 | 36.227 | 72.239 | −3.494 | 1.00 | 157.10 |
| 3708 | O | VAL | B | 83 | 35.120 | 72.455 | −3.992 | 1.00 | 157.10 |
| 3709 | N | PHE | B | 84 | 37.344 | 72.845 | −3.895 | 1.00 | 122.91 |
| 3710 | CA | PHE | B | 84 | 37.331 | 73.921 | −4.875 | 1.00 | 122.91 |
| 3711 | CB | PHE | B | 84 | 37.654 | 75.240 | −4.180 | 1.00 | 156.44 |
| 3712 | CG | PHE | B | 84 | 36.687 | 75.629 | −3.106 | 1.00 | 156.44 |
| 3713 | CD1 | PHE | B | 84 | 36.773 | 75.093 | −1.821 | 1.00 | 156.44 |
| 3714 | CD2 | PHE | B | 84 | 35.696 | 76.562 | −3.382 | 1.00 | 156.44 |
| 3715 | CE1 | PHE | B | 84 | 35.885 | 75.485 | −0.833 | 1.00 | 156.44 |
| 3716 | CE2 | PHE | B | 84 | 34.805 | 76.958 | −2.401 | 1.00 | 156.44 |
| 3717 | CZ | PHE | B | 84 | 34.903 | 76.419 | −1.121 | 1.00 | 156.44 |
| 3718 | C | PHE | B | 84 | 38.259 | 73.854 | −6.045 | 1.00 | 122.91 |
| 3719 | O | PHE | B | 84 | 39.300 | 73.198 | −6.007 | 1.00 | 122.91 |
| 3720 | N | SER | B | 85 | 37.884 | 74.614 | −7.064 | 1.00 | 152.63 |
| 3721 | CA | SER | B | 85 | 38.690 | 74.765 | −8.267 | 1.00 | 152.63 |
| 3722 | CB | SER | B | 85 | 38.054 | 74.080 | −9.467 | 1.00 | 143.15 |
| 3723 | OG | SER | B | 85 | 38.872 | 74.255 | −10.617 | 1.00 | 143.15 |
| 3724 | C | SER | B | 85 | 38.741 | 76.262 | −8.523 | 1.00 | 152.63 |
| 3725 | O | SER | B | 85 | 37.715 | 76.882 | −8.796 | 1.00 | 152.63 |
| 3726 | N | ASP | B | 86 | 39.933 | 76.836 | −8.412 | 1.00 | 139.36 |
| 3727 | CA | ASP | B | 86 | 40.120 | 78.263 | −8.613 | 1.00 | 139.36 |
| 3728 | CB | ASP | B | 86 | 39.314 | 79.047 | −7.577 | 1.00 | 472.94 |
| 3729 | CG | ASP | B | 86 | 38.724 | 80.313 | −8.143 | 1.00 | 172.94 |
| 3730 | OD1 | ASP | B | 86 | 39.476 | 81.114 | −8.744 | 1.00 | 172.94 |
| 3731 | OD2 | ASP | B | 86 | 37.505 | 80.511 | −7.977 | 1.00 | 172.94 |
| 3732 | C | ASP | B | 86 | 41.606 | 78.609 | −8.458 | 1.00 | 139.36 |
| 3733 | O | ASP | B | 86 | 42.379 | 77.794 | −7.963 | 1.00 | 139.36 |
| 3734 | N | TRP | B | 87 | 42.003 | 79.814 | −8.863 | 1.00 | 121.62 |
| 3735 | CA | TRP | B | 87 | 43.393 | 80.224 | −8.745 | 1.00 | 121.62 |
| 3736 | CB | TRP | B | 87 | 43.617 | 81.544 | −9.459 | 1.00 | 247.13 |
| 3737 | CG | TRP | B | 87 | 43.989 | 81.344 | −10.891 | 1.00 | 247.13 |
| 3738 | CD2 | TRP | B | 87 | 43.103 | 81.328 | −12.013 | 1.00 | 247.13 |
| 3739 | CE2 | TRP | B | 87 | 43.881 | 81.063 | −13.164 | 1.00 | 247.13 |
| 3740 | CE3 | TRP | B | 87 | 41.722 | 81.521 | −12.166 | 1.00 | 247.13 |
| 3741 | CD1 | TRP | B | 87 | 45.237 | 81.085 | −11.386 | 1.00 | 247.13 |
| 3742 | NE1 | TRP | B | 87 | 45.183 | 80.918 | −12.752 | 1.00 | 247.13 |
| 3743 | CZ2 | TRP | B | 87 | 43.328 | 80.982 | −14.445 | 1.00 | 247.13 |
| 3744 | CZ3 | TRP | B | 87 | 41.170 | 81.442 | −13.443 | 1.00 | 247.13 |
| 3745 | CH2 | TRP | B | 87 | 41.973 | 81.171 | −14.563 | 1.00 | 247.13 |
| 3746 | C | TRP | B | 87 | 43.788 | 80.336 | −7.288 | 1.00 | 121.62 |
| 3747 | O | TRP | B | 87 | 44.771 | 79.726 | −6.870 | 1.00 | 121.62 |
| 3748 | N | LEU | B | 88 | 43.030 | 81.106 | −6.510 | 1.00 | 133.11 |
| 3749 | CA | LEU | B | 88 | 43.333 | 81.246 | −5.092 | 1.00 | 133.11 |
| 3750 | CB | LEU | B | 88 | 43.787 | 82.678 | −4.789 | 1.00 | 136.45 |
| 3751 | CG | LEU | B | 88 | 45.105 | 83.123 | −5.444 | 1.00 | 136.45 |
| 3752 | CD1 | LEU | B | 88 | 45.489 | 84.525 | −4.973 | 1.00 | 136.45 |
| 3753 | CD2 | LEU | B | 88 | 46.196 | 82.131 | −5.093 | 1.00 | 136.45 |
| 3754 | C | LEU | B | 88 | 42.137 | 80.876 | −4.219 | 1.00 | 133.11 |
| 3755 | O | LEU | B | 88 | 40.985 | 81.159 | −4.577 | 1.00 | 133.11 |
| 3756 | N | LEU | B | 89 | 42.413 | 80.222 | −3.086 | 1.00 | 126.96 |
| 3757 | CA | LEU | B | 89 | 41.363 | 79.834 | −2.143 | 1.00 | 126.96 |
| 3758 | CB | LEU | B | 89 | 41.152 | 78.335 | −2.153 | 1.00 | 166.49 |
| 3759 | CG | LEU | B | 89 | 40.113 | 77.865 | −1.131 | 1.00 | 166.49 |
| 3760 | CD1 | LEU | B | 89 | 38.812 | 78.646 | −1.306 | 1.00 | 166.49 |
| 3761 | CD2 | LEU | B | 89 | 39.881 | 76.365 | −1.300 | 1.00 | 166.49 |
| 3762 | C | LEU | B | 89 | 41.777 | 80.255 | −0.749 | 1.00 | 126.96 |
| 3763 | O | LEU | B | 89 | 42.900 | 79.978 | −0.336 | 1.00 | 126.96 |
| 3764 | N | LEU | B | 90 | 40.883 | 80.929 | −0.029 | 1.00 | 113.68 |
| 3765 | CA | LEU | B | 90 | 41.196 | 81.391 | 1.326 | 1.00 | 113.68 |
| 3766 | CB | LEU | B | 90 | 40.422 | 82.674 | 1.646 | 1.00 | 125.93 |
| 3767 | CG | LEU | B | 90 | 40.536 | 83.133 | 3.101 | 1.00 | 125.93 |
| 3768 | CD1 | LEU | B | 90 | 41.984 | 83.406 | 3.418 | 1.00 | 125.93 |
| 3769 | CD2 | LEU | B | 90 | 39.691 | 84.379 | 3.334 | 1.00 | 125.93 |
| 3770 | C | LEU | B | 90 | 40.856 | 80.326 | 2.356 | 1.00 | 113.68 |
| 3771 | O | LEU | B | 90 | 39.693 | 80.010 | 2.566 | 1.00 | 113.68 |
| 3772 | N | GLN | B | 91 | 41.870 | 79.780 | 3.010 | 1.00 | 98.97 |
| 3773 | CA | GLN | B | 91 | 41.644 | 78.731 | 4.000 | 1.00 | 98.97 |
| 3774 | CB | GLN | B | 91 | 42.662 | 77.614 | 3.842 | 1.00 | 171.07 |
| 3775 | CG | GLN | B | 91 | 42.650 | 76.964 | 2.481 | 1.00 | 171.07 |
| 3776 | CD | GLN | B | 91 | 43.685 | 75.865 | 2.363 | 1.00 | 171.07 |
| 3777 | OE1 | GLN | B | 91 | 44.878 | 76.095 | 2.555 | 1.00 | 171.07 |
| 3778 | NE2 | GLN | B | 91 | 43.232 | 74.660 | 2.046 | 1.00 | 171.07 |
| 3779 | C | GLN | B | 91 | 41.707 | 79.237 | 5.422 | 1.00 | 98.97 |
| 3780 | O | GLN | B | 91 | 42.592 | 80.023 | 5.780 | 1.00 | 98.97 |
| 3781 | N | ALA | B | 92 | 40.769 | 78.773 | 6.244 | 1.00 | 123.57 |
| 3782 | CA | ALA | B | 92 | 40.707 | 79.189 | 7.645 | 1.00 | 123.57 |
| 3783 | CB | ALA | B | 92 | 39.422 | 79.967 | 7.914 | 1.00 | 155.46 |
| 3784 | C | ALA | B | 92 | 40.774 | 77.974 | 8.537 | 1.00 | 123.57 |
| 3785 | O | ALA | B | 92 | 40.215 | 76.918 | 8.208 | 1.00 | 123.57 |
| 3786 | N | SER | B | 93 | 41.473 | 78.126 | 9.662 | 1.00 | 119.49 |
| 3787 | CA | SER | B | 93 | 41.631 | 77.046 | 10.652 | 1.00 | 119.49 |
| 3788 | CB | SER | B | 93 | 42.377 | 77.561 | 11.897 | 1.00 | 144.43 |
| 3789 | OG | SER | B | 93 | 41.794 | 78.730 | 12.443 | 1.00 | 144.43 |
| 3790 | C | SER | B | 93 | 40.238 | 76.547 | 11.026 | 1.00 | 119.49 |
| 3791 | O | SER | B | 93 | 39.891 | 75.389 | 10.783 | 1.00 | 119.49 |
| 3792 | N | ALA | B | 94 | 39.447 | 77.448 | 11.605 | 1.00 | 139.25 |
| 3793 | CA | ALA | B | 94 | 38.072 | 77.173 | 11.989 | 1.00 | 139.25 |
| 3794 | CB | ALA | B | 94 | 37.961 | 77.053 | 13.497 | 1.00 | 173.88 |
| 3795 | C | ALA | B | 94 | 37.289 | 78.378 | 11.484 | 1.00 | 139.25 |
| 3796 | O | ALA | B | 94 | 37.843 | 79.465 | 11.346 | 1.00 | 139.25 |
| 3797 | N | GLU | B | 95 | 36.010 | 78.197 | 11.188 | 1.00 | 153.31 |
| 3798 | CA | GLU | B | 95 | 35.217 | 79.312 | 10.694 | 1.00 | 153.31 |
| 3799 | CB | GLU | B | 95 | 34.219 | 78.813 | 9.648 | 1.00 | 193.17 |
| 3800 | CG | GLU | B | 95 | 34.894 | 78.235 | 8.419 | 1.00 | 193.17 |
| 3801 | CD | GLU | B | 95 | 33.929 | 77.974 | 7.284 | 1.00 | 193.17 |
| 3802 | OE1 | GLU | B | 95 | 34.372 | 77.478 | 6.230 | 1.00 | 193.17 |
| 3803 | OE2 | GLU | B | 95 | 32.727 | 78.269 | 7.442 | 1.00 | 193.17 |
| 3804 | C | GLU | B | 95 | 34.493 | 80.058 | 11.817 | 1.00 | 153.31 |
| 3805 | O | GLU | B | 95 | 34.153 | 81.241 | 11.678 | 1.00 | 153.31 |
| 3806 | N | VAL | B | 96 | 34.272 | 79.364 | 12.930 | 1.00 | 119.30 |
| 3807 | CA | VAL | B | 96 | 33.599 | 79.942 | 14.087 | 1.00 | 119.30 |
| 3808 | CB | VAL | B | 96 | 32.262 | 79.239 | 14.355 | 1.00 | 135.82 |
| 3809 | CG1 | VAL | B | 96 | 31.387 | 80.129 | 15.250 | 1.00 | 135.82 |
| 3810 | CG2 | VAL | B | 96 | 31.566 | 78.905 | 13.045 | 1.00 | 135.82 |
| 3811 | C | VAL | B | 96 | 34.482 | 79.788 | 15.330 | 1.00 | 119.30 |
| 3812 | O | VAL | B | 96 | 34.967 | 78.690 | 15.622 | 1.00 | 119.30 |
| 3813 | N | VAL | B | 97 | 34.664 | 80.871 | 16.082 | 1.00 | 139.44 |
| 3814 | CA | VAL | B | 97 | 35.530 | 80.799 | 17.247 | 1.00 | 139.44 |
| 3815 | CB | VAL | B | 97 | 36.882 | 81.401 | 16.927 | 1.00 | 116.73 |
| 3816 | CG1 | VAL | B | 97 | 37.890 | 80.880 | 17.910 | 1.00 | 116.73 |
| 3817 | CG2 | VAL | B | 97 | 37.283 | 81.074 | 15.506 | 1.00 | 116.73 |
| 3818 | C | VAL | B | 97 | 35.070 | 81.451 | 18.543 | 1.00 | 139.44 |
| 3819 | O | VAL | B | 97 | 34.355 | 82.445 | 18.528 | 1.00 | 139.44 |
| 3820 | N | MET | B | 98 | 35.528 | 80.882 | 19.659 | 1.00 | 148.58 |
| 3821 | CA | MET | B | 98 | 35.228 | 81.363 | 21.015 | 1.00 | 148.58 |
| 3822 | CB | MET | B | 98 | 35.399 | 80.224 | 22.034 | 1.00 | 249.69 |
| 3823 | CG | MET | B | 98 | 34.439 | 79.059 | 21.898 | 1.00 | 249.69 |
| 3824 | SD | MET | B | 98 | 32.799 | 79.444 | 22.531 | 1.00 | 249.69 |
| 3825 | CE | MET | B | 98 | 33.078 | 79.331 | 24.290 | 1.00 | 249.69 |
| 3826 | C | MET | B | 98 | 36.217 | 82.467 | 21.365 | 1.00 | 148.58 |
| 3827 | O | MET | B | 98 | 37.425 | 82.250 | 21.304 | 1.00 | 148.58 |
| 3828 | N | GLU | B | 99 | 35.721 | 83.636 | 21.756 | 1.00 | 152.43 |
| 3829 | CA | GLU | B | 99 | 36.604 | 84.754 | 22.102 | 1.00 | 152.43 |
| 3830 | CB | GLU | B | 99 | 35.865 | 85.773 | 22.970 | 1.00 | 244.45 |
| 3831 | CG | GLU | B | 99 | 36.447 | 87.177 | 22.890 | 1.00 | 244.45 |
| 3832 | CD | GLU | B | 99 | 35.891 | 88.095 | 23.954 | 1.00 | 244.45 |
| 3833 | OE1 | GLU | B | 99 | 34.681 | 87.989 | 24.264 | 1.00 | 244.45 |
| 3834 | OE2 | GLU | B | 99 | 36.663 | 88.930 | 24.468 | 1.00 | 244.45 |
| 3835 | C | GLU | B | 99 | 37.831 | 84.264 | 22.859 | 1.00 | 152.43 |
| 3836 | O | GLU | B | 99 | 37.708 | 83.591 | 23.877 | 1.00 | 152.43 |
| 3837 | N | GLY | B | 100 | 39.015 | 84.586 | 22.363 | 1.00 | 146.37 |
| 3838 | CA | GLY | B | 100 | 40.216 | 84.144 | 23.053 | 1.00 | 146.37 |
| 3839 | C | GLY | B | 100 | 41.006 | 83.025 | 22.397 | 1.00 | 146.37 |
| 3840 | O | GLY | B | 100 | 42.205 | 82.880 | 22.659 | 1.00 | 146.37 |
| 3841 | N | GLN | B | 101 | 40.353 | 82.237 | 21.552 | 1.00 | 128.49 |
| 3842 | CA | GLN | B | 101 | 41.009 | 81.129 | 20.851 | 1.00 | 128.49 |
| 3843 | CB | GLN | B | 101 | 39.964 | 80.177 | 20.290 | 1.00 | 190.16 |
| 3844 | CG | GLN | B | 101 | 39.253 | 79.391 | 21.346 | 1.00 | 190.16 |
| 3845 | CD | GLN | B | 101 | 40.226 | 78.874 | 22.370 | 1.00 | 190.16 |
| 3846 | OE1 | GLN | B | 101 | 40.792 | 79.645 | 23.140 | 1.00 | 190.16 |
| 3847 | NE2 | GLN | B | 101 | 40.441 | 77.565 | 22.378 | 1.00 | 190.16 |
| 3848 | C | GLN | B | 101 | 41.952 | 81.596 | 19.731 | 1.00 | 128.49 |
| 3849 | O | GLN | B | 101 | 42.038 | 82.780 | 19.407 | 1.00 | 128.49 |
| 3850 | N | PRO | B | 102 | 42.675 | 80.637 | 19.115 | 1.00 | 164.10 |
| 3851 | CD | PRO | B | 102 | 42.886 | 79.258 | 19.540 | 1.00 | 192.64 |
| 3852 | CA | PRO | B | 102 | 43.581 | 81.010 | 18.018 | 1.00 | 164.10 |
| 3853 | CB | PRO | B | 102 | 44.669 | 79.950 | 18.152 | 1.00 | 192.64 |
| 3854 | CG | PRO | B | 102 | 43.863 | 78.733 | 18.490 | 1.00 | 192.64 |
| 3855 | C | PRO | B | 102 | 42.928 | 80.983 | 16.652 | 1.00 | 164.10 |
| 3856 | O | PRO | B | 102 | 42.017 | 80.208 | 16.389 | 1.00 | 164.10 |
| 3857 | N | LEU | B | 103 | 43.418 | 81.831 | 15.761 | 1.00 | 147.70 |
| 3858 | CA | LEU | B | 103 | 42.870 | 81.913 | 14.425 | 1.00 | 147.70 |
| 3859 | CB | LEU | B | 103 | 41.981 | 83.136 | 14.328 | 1.00 | 111.28 |
| 3860 | CG | LEU | B | 103 | 41.244 | 83.102 | 13.004 | 1.00 | 111.28 |
| 3861 | CD1 | LEU | B | 103 | 40.261 | 81.951 | 13.100 | 1.00 | 111.28 |
| 3862 | CD2 | LEU | B | 103 | 40.546 | 84.415 | 12.716 | 1.00 | 111.28 |
| 3863 | C | LEU | B | 103 | 43.939 | 81.997 | 13.331 | 1.00 | 147.70 |
| 3864 | O | LEU | B | 103 | 44.797 | 82.868 | 13.379 | 1.00 | 147.70 |
| 3865 | N | PHE | B | 104 | 43.890 | 81.112 | 12.339 | 1.00 | 122.26 |
| 3866 | CA | PHE | B | 104 | 44.880 | 81.157 | 11.261 | 1.00 | 122.26 |
| 3867 | CB | PHE | B | 104 | 45.820 | 79.951 | 11.311 | 1.00 | 249.69 |
| 3868 | CG | PHE | B | 104 | 46.501 | 79.766 | 12.627 | 1.00 | 249.69 |
| 3869 | CD1 | PHE | B | 104 | 45.816 | 79.216 | 13.707 | 1.00 | 249.69 |
| 3870 | CD2 | PHE | B | 104 | 47.831 | 80.137 | 12.793 | 1.00 | 249.69 |
| 3871 | CE1 | PHE | B | 104 | 46.444 | 79.041 | 14.939 | 1.00 | 249.69 |
| 3872 | CE2 | PHE | B | 104 | 48.467 | 79.967 | 14.020 | 1.00 | 249.69 |
| 3873 | CZ | PHE | B | 104 | 47.773 | 79.416 | 15.096 | 1.00 | 249.69 |
| 3874 | C | PHE | B | 104 | 44.227 | 81.185 | 9.885 | 1.00 | 122.26 |
| 3875 | O | PHE | B | 104 | 43.451 | 80.290 | 9.546 | 1.00 | 122.26 |
| 3876 | N | LEU | B | 105 | 44.541 | 82.207 | 9.093 | 1.00 | 122.11 |
| 3877 | CA | LEU | B | 105 | 44.001 | 82.309 | 7.743 | 1.00 | 122.11 |
| 3878 | CB | LEU | B | 105 | 43.390 | 83.676 | 7.495 | 1.00 | 103.82 |
| 3879 | CG | LEU | B | 105 | 42.270 | 84.000 | 8.466 | 1.00 | 103.32 |
| 3880 | CD1 | LEU | B | 105 | 41.606 | 85.309 | 8.040 | 1.00 | 103.82 |
| 3881 | CD2 | LEU | B | 105 | 41.254 | 82.847 | 8.512 | 1.00 | 103.82 |
| 3882 | C | LEU | B | 105 | 45.130 | 82.083 | 6.776 | 1.00 | 122.11 |
| 3883 | O | LEU | B | 105 | 46.272 | 82.387 | 7.065 | 1.00 | 122.11 |
| 3884 | N | ARG | B | 106 | 44.820 | 81.560 | 5.608 | 1.00 | 131.12 |
| 3885 | CA | ARC | B | 106 | 45.870 | 81.289 | 4.645 | 1.00 | 131.12 |
| 3886 | CB | ARG | B | 106 | 46.287 | 79.828 | 4.792 | 1.00 | 175.38 |
| 3887 | CG | ARG | B | 106 | 47.384 | 79.387 | 3.874 | 1.00 | 175.38 |
| 3888 | CD | ARG | B | 106 | 47.593 | 77.897 | 3.980 | 1.00 | 175.38 |
| 3889 | NE | ARG | B | 106 | 48.544 | 77.460 | 2.972 | 1.00 | 175.38 |
| 3890 | CZ | ARG | B | 106 | 48.536 | 76.259 | 2.404 | 1.00 | 175.38 |
| 3891 | NH1 | ARG | B | 106 | 47.618 | 75.361 | 2.743 | 1.00 | 175.38 |
| 3892 | NH2 | ARG | B | 106 | 49.438 | 75.963 | 1.477 | 1.00 | 175.38 |
| 3893 | C | ARG | B | 106 | 45.418 | 81.578 | 3.220 | 1.00 | 131.12 |
| 3894 | O | ARG | B | 106 | 44.374 | 81.096 | 2.784 | 1.00 | 131.12 |
| 3895 | N | CYS | B | 107 | 46.184 | 82.394 | 2.507 | 1.00 | 139.30 |
| 3896 | CA | CYS | B | 107 | 45.852 | 82.704 | 1.124 | 1.00 | 139.30 |
| 3897 | C | CYS | B | 107 | 46.515 | 81.564 | 0.378 | 1.00 | 139.30 |
| 3898 | O | CYS | B | 107 | 47.747 | 81.560 | 0.228 | 1.00 | 139.30 |
| 3899 | CB | CYS | B | 107 | 46.472 | 84.034 | 0.698 | 1.00 | 152.04 |
| 3900 | SG | CYS | B | 107 | 45.853 | 84.704 | −0.878 | 1.00 | 152.04 |
| 3901 | N | HIS | B | 108 | 45.703 | 80.604 | −0.073 | 1.00 | 155.54 |
| 3902 | CA | HIS | B | 108 | 46.205 | 79.419 | −0.771 | 1.00 | 155.54 |
| 3903 | CB | HIS | B | 108 | 45.429 | 78.180 | −0.319 | 1.00 | 137.70 |
| 3904 | CG | HIS | B | 108 | 46.047 | 76.880 | −0.745 | 1.00 | 137.70 |
| 3905 | CD2 | HIS | B | 108 | 45.514 | 75.802 | −1.357 | 1.00 | 137.70 |
| 3906 | ND1 | HIS | B | 108 | 47.366 | 76.564 | −0.486 | 1.00 | 137.70 |
| 3907 | CE1 | HIS | B | 108 | 47.609 | 75.341 | −0.919 | 1.00 | 137.70 |
| 3908 | NE2 | HIS | B | 108 | 46.506 | 74.854 | −1.453 | 1.00 | 137.70 |
| 3909 | C | HIS | B | 108 | 46.182 | 79.487 | −2.289 | 1.00 | 155.54 |
| 3910 | O | HIS | B | 108 | 45.137 | 79.731 | −2.900 | 1.00 | 155.54 |
| 3911 | N | GLY | B | 109 | 47.351 | 79.253 | −2.881 | 1.00 | 164.07 |
| 3912 | CA | GLY | B | 109 | 47.480 | 79.271 | −4.321 | 1.00 | 164.07 |
| 3913 | C | GLY | B | 109 | 47.090 | 77.915 | −4.866 | 1.00 | 164.07 |
| 3914 | O | GLY | B | 109 | 46.967 | 76.949 | −4.098 | 1.00 | 164.07 |
| 3915 | N | TRP | B | 110 | 46.886 | 77.833 | −6.180 | 1.00 | 153.07 |
| 3916 | CA | TRP | B | 110 | 46.506 | 76.568 | −6.812 | 1.00 | 153.07 |
| 3917 | CB | TRP | B | 110 | 45.719 | 76.839 | −8.097 | 1.00 | 171.40 |
| 3918 | CG | TRP | B | 110 | 45.467 | 75.611 | −8.920 | 1.00 | 171.40 |
| 3919 | CD2 | TRP | B | 110 | 44.256 | 74.833 | −8.973 | 1.00 | 171.40 |
| 3920 | CE2 | TRP | B | 110 | 44.496 | 73.764 | −9.855 | 1.00 | 171.40 |
| 3921 | CE3 | TRP | B | 110 | 42.998 | 74.937 | −8.353 | 1.00 | 171.40 |
| 3922 | CD1 | TRP | B | 110 | 46.348 | 75.000 | −9.745 | 1.00 | 171.40 |
| 3923 | NE1 | TRP | B | 110 | 45.778 | 73.889 | −10.311 | 1.00 | 171.40 |
| 3924 | CZ2 | TRP | B | 110 | 43.521 | 72.799 | −10.140 | 1.00 | 171.40 |
| 3925 | CZ3 | TRP | B | 110 | 42.026 | 73.974 | −8.639 | 1.00 | 171.40 |
| 3926 | CH2 | TRP | B | 110 | 42.297 | 72.921 | −9.526 | 1.00 | 171.40 |
| 3927 | C | TRP | B | 110 | 47.743 | 75.723 | −7.098 | 1.00 | 153.07 |
| 3928 | O | TRP | B | 110 | 48.833 | 76.257 | −7.296 | 1.00 | 153.07 |
| 3929 | N | ARG | B | 111 | 47.567 | 74.404 | −7.104 | 1.00 | 188.19 |
| 3930 | CA | ARG | B | 111 | 48.671 | 73.481 | −7.333 | 1.00 | 188.19 |
| 3931 | CB | ARG | B | 111 | 49.124 | 73.522 | −8.790 | 1.00 | 249.69 |
| 3932 | CG | ARG | B | 111 | 48.394 | 72.532 | −9.682 | 1.00 | 249.69 |
| 3933 | CD | ARG | B | 111 | 49.101 | 72.359 | −11.018 | 1.00 | 249.69 |
| 3934 | NE | ARG | B | 111 | 49.095 | 70.961 | −11.448 | 1.00 | 249.69 |
| 3935 | CZ | ARG | B | 111 | 49.641 | 69.965 | −10.751 | 1.00 | 249.69 |
| 3936 | NH1 | ARG | B | 111 | 50.241 | 70.210 | −9.587 | 1.00 | 249.69 |
| 3937 | NH2 | ARG | B | 111 | 49.594 | 68.722 | −11.216 | 1.00 | 249.69 |
| 3938 | C | ARG | B | 111 | 49.845 | 73.814 | −6.431 | 1.00 | 188.19 |
| 3939 | O | ARG | B | 111 | 50.999 | 73.588 | −6.787 | 1.00 | 188.19 |
| 3940 | N | ASN | B | 112 | 49.534 | 74.350 | −5.260 | 1.00 | 160.66 |
| 3941 | CA | ASN | B | 112 | 50.542 | 74.729 | −4.291 | 1.00 | 160.66 |
| 3942 | CB | ASN | B | 112 | 51.146 | 73.478 | −3.648 | 1.00 | 209.47 |
| 3943 | CG | ASN | B | 112 | 51.910 | 73.791 | −2.372 | 1.00 | 209.47 |
| 3944 | OD1 | ASN | B | 112 | 52.226 | 74.947 | −2.083 | 1.00 | 209.47 |
| 3945 | ND2 | ASN | B | 112 | 52.221 | 72.757 | −1.607 | 1.00 | 209.47 |
| 3946 | C | ASN | B | 112 | 51.652 | 75.583 | −4.931 | 1.00 | 160.66 |
| 3947 | O | ASN | B | 112 | 52.808 | 75.522 | −4.489 | 1.00 | 160.66 |
| 3948 | N | TRP | B | 113 | 51.319 | 76.368 | −5.966 | 1.00 | 180.98 |
| 3949 | CA | TRP | B | 113 | 52.321 | 77.223 | −6.608 | 1.00 | 180.98 |
| 3950 | CB | TRP | B | 113 | 51.781 | 77.888 | −7.863 | 1.00 | 248.40 |
| 3951 | CG | TRP | B | 113 | 51.824 | 77.037 | −9.064 | 1.00 | 248.40 |
| 3952 | CD2 | TRP | B | 113 | 50.876 | 77.029 | −10.131 | 1.00 | 248.40 |
| 3953 | CE2 | TRP | B | 113 | 51.344 | 76.102 | −11.092 | 1.00 | 248.40 |
| 3954 | CE3 | TRP | B | 113 | 49.678 | 77.717 | −10.370 | 1.00 | 248.40 |
| 3955 | CD1 | TRP | B | 113 | 52.799 | 76.145 | −9.405 | 1.00 | 248.40 |
| 3956 | NE1 | TRP | B | 113 | 52.513 | 75.582 | −10.623 | 1.00 | 248.40 |
| 3957 | CZ2 | TRP | B | 113 | 50.639 | 75.845 | −12.289 | 1.00 | 248.40 |
| 3958 | CZ3 | TRP | B | 113 | 48.984 | 77.461 | −11.557 | 1.00 | 248.40 |
| 3959 | CH2 | TRP | B | 113 | 49.472 | 76.530 | −12.504 | 1.00 | 248.40 |
| 3960 | C | TRP | B | 113 | 52.741 | 78.311 | −5.641 | 1.00 | 180.98 |
| 3961 | O | TRP | B | 113 | 52.414 | 78.261 | −4.452 | 1.00 | 180.98 |
| 3962 | N | ASP | B | 114 | 53.465 | 79.305 | −6.144 | 1.00 | 240.61 |
| 3963 | CA | ASP | B | 114 | 53.905 | 80.407 | −5.292 | 1.00 | 240.61 |
| 3964 | CB | ASP | B | 114 | 55.427 | 80.596 | −5.390 | 1.00 | 249.69 |
| 3965 | CG | ASP | B | 114 | 56.207 | 79.633 | −4.489 | 1.00 | 249.69 |
| 3966 | OD1 | ASP | B | 114 | 55.981 | 79.644 | −3.260 | 1.00 | 249.69 |
| 3967 | OD2 | ASP | B | 114 | 57.054 | 78.868 | −5.007 | 1.00 | 249.69 |
| 3968 | C | ASP | B | 114 | 53.185 | 81.707 | −5.655 | 1.00 | 240.61 |
| 3969 | O | ASP | 8 | 114 | 53.064 | 82.062 | −6.841 | 1.00 | 240.61 |
| 3970 | N | VAL | B | 115 | 52.691 | 82.403 | −4.627 | 1.00 | 153.55 |
| 3971 | CA | VAL | B | 115 | 51.976 | 83.665 | −4.828 | 1.00 | 153.55 |
| 3972 | CB | VAL | B | 115 | 50.603 | 83.666 | −4.135 | 1.00 | 146.14 |
| 3973 | CG1 | VAL | B | 115 | 49.770 | 84.824 | −4.640 | 1.00 | 146.14 |
| 3974 | CG2 | VAL | B | 115 | 49.887 | 82.359 | −4.385 | 1.00 | 146.14 |
| 3975 | C | VAL | B | 115 | 52.790 | 84.823 | −4.256 | 1.00 | 153.55 |
| 3976 | O | VAL | B | 115 | 53.410 | 84.708 | −3.194 | 1.00 | 153.55 |
| 3977 | N | TYR | B | 116 | 52.781 | 85.939 | −4.975 | 1.00 | 165.97 |
| 3978 | CA | TYR | B | 116 | 53.518 | 87.128 | −4.575 | 1.00 | 165.97 |
| 3979 | CB | TYR | B | 116 | 54.595 | 87.441 | −5.619 | 1.00 | 249.69 |
| 3980 | CG | TYR | B | 116 | 55.660 | 86.371 | −5.722 | 1.00 | 249.69 |
| 3981 | CD1 | TYR | B | 116 | 55.612 | 85.393 | −6.716 | 1.00 | 249.69 |
| 3982 | CE1 | TYR | B | 116 | 56.582 | 84.386 | −6.784 | 1.00 | 249.69 |
| 3983 | CD2 | TYR | B | 116 | 56.700 | 86.319 | −4.802 | 1.00 | 249.69 |
| 3984 | CE2 | TYR | B | 116 | 57.671 | 85.322 | −4.857 | 1.00 | 249.69 |
| 3985 | CZ | TYR | B | 116 | 57.611 | 84.360 | −5.847 | 1.00 | 249.69 |
| 3986 | OH | TYR | B | 116 | 58.575 | 83.375 | −5.898 | 1.00 | 249.69 |
| 3987 | C | TYR | B | 116 | 52.593 | 88.332 | −4.405 | 1.00 | 165.97 |
| 3988 | O | TYR | B | 116 | 51.423 | 88.278 | −4.782 | 1.00 | 165.97 |
| 3989 | N | LYS | B | 117 | 53.129 | 89.413 | −3.840 | 1.00 | 123.89 |
| 3990 | CA | LYS | B | 117 | 52.369 | 90.641 | −3.609 | 1.00 | 123.89 |
| 3991 | CB | LYS | B | 117 | 52.154 | 91.413 | −4.924 | 1.00 | 248.40 |
| 3992 | CG | LYS | B | 117 | 53.310 | 92.323 | −5.333 | 1.00 | 243.40 |
| 3993 | CD | LYS | B | 117 | 52.850 | 93.384 | −6.340 | 1.00 | 248.40 |
| 3994 | CE | LYS | B | 117 | 51.729 | 94.253 | −5.761 | 1.00 | 248.40 |
| 3995 | NZ | LYS | B | 117 | 51.232 | 95.311 | −6.687 | 1.00 | 248.40 |
| 3996 | C | LYS | B | 117 | 51.019 | 90.378 | −2.948 | 1.00 | 123.89 |
| 3997 | O | LYS | B | 117 | 49.972 | 90.844 | −3.409 | 1.00 | 123.89 |
| 3998 | N | VAL | B | 118 | 51.055 | 89.650 | −1.846 | 1.00 | 126.95 |
| 3999 | CA | VAL | B | 118 | 49.843 | 89.314 | −1.142 | 1.00 | 126.95 |
| 4000 | CB | VAL | B | 118 | 50.033 | 87.999 | −0.408 | 1.00 | 114.58 |
| 4001 | CG1 | VAL | B | 118 | 49.078 | 87.892 | 0.760 | 1.00 | 114.58 |
| 4002 | CG2 | VAL | B | 118 | 49.789 | 86.867 | −1.372 | 1.00 | 114.58 |
| 4003 | C | VAL | B | 118 | 49.299 | 90.352 | −0.177 | 1.00 | 126.95 |
| 4004 | O | VAL | B | 118 | 50.051 | 91.007 | 0.547 | 1.00 | 126.95 |
| 4005 | N | ILE | B | 119 | 47.972 | 90.477 | −0.179 | 1.00 | 120.13 |
| 4006 | CA | ILE | B | 119 | 47.244 | 91.402 | 0.686 | 1.00 | 120.13 |
| 4007 | CB | ILE | B | 119 | 46.813 | 92.633 | −0.073 | 1.00 | 113.05 |
| 4008 | CG2 | ILE | B | 119 | 46.149 | 93.628 | 0.860 | 1.00 | 113.05 |
| 4009 | CG1 | ILE | B | 119 | 48.021 | 93.233 | −0.750 | 1.00 | 113.05 |
| 4010 | CD1 | ILE | B | 119 | 47.645 | 94.049 | −1.930 | 1.00 | 113.05 |
| 4011 | C | ILE | B | 119 | 45.971 | 90.716 | 1.152 | 1.00 | 120.13 |
| 4012 | O | ILE | B | 119 | 45.244 | 90.135 | 0.347 | 1.00 | 120.13 |
| 4013 | N | TYR | B | 120 | 45.700 | 90.761 | 2.448 | 1.00 | 131.55 |
| 4014 | CA | TYR | B | 120 | 44.475 | 90.162 | 2.952 | 1.00 | 131.55 |
| 4015 | CB | TYR | B | 120 | 44.704 | 89.457 | 4.286 | 1.00 | 143.62 |
| 4016 | CG | TYR | B | 120 | 45.550 | 88.215 | 4.186 | 1.00 | 143.62 |
| 4017 | CD1 | TYR | B | 120 | 46.935 | 88.292 | 4.258 | 1.00 | 143.62 |
| 4018 | CE1 | TYR | B | 120 | 47.725 | 87.153 | 4.175 | 1.00 | 143.62 |
| 4019 | CD2 | TYR | B | 120 | 44.966 | 86.963 | 4.019 | 1.00 | 143.62 |
| 4020 | CE2 | TYR | B | 120 | 45.733 | 85.821 | 3.930 | 1.00 | 143.62 |
| 4021 | CZ | TYR | B | 120 | 47.117 | 85.918 | 4.010 | 1.00 | 143.62 |
| 4022 | OH | TYR | B | 120 | 47.898 | 84.776 | 3.937 | 1.00 | 143.62 |
| 4023 | C | TYR | B | 120 | 43.506 | 91.304 | 3.146 | 1.00 | 131.55 |
| 4024 | O | TYR | B | 120 | 43.919 | 92.426 | 3.396 | 1.00 | 131.55 |
| 4025 | N | TYR | B | 121 | 42.221 | 91.024 | 3.013 | 1.00 | 134.10 |
| 4026 | CA | TYR | B | 121 | 41.210 | 92.047 | 3.197 | 1.00 | 134.10 |
| 4027 | CB | TYR | B | 121 | 40.572 | 92.433 | 1.867 | 1.00 | 128.02 |
| 4028 | CG | TYR | B | 121 | 41.444 | 93.147 | 0.886 | 1.00 | 128.02 |
| 4029 | CD1 | TYR | B | 121 | 42.598 | 92.562 | 0.407 | 1.00 | 128.02 |
| 4030 | CE1 | TYR | B | 121 | 43.370 | 93.184 | −0.612 | 1.00 | 128.02 |
| 4031 | CD2 | TYR | B | 121 | 41.060 | 94.376 | 0.350 | 1.00 | 128.02 |
| 4032 | CE2 | TYR | B | 121 | 41.810 | 95.003 | −0.661 | 1.00 | 128.02 |
| 4033 | CZ | TYR | B | 121 | 42.970 | 94.395 | −1.142 | 1.00 | 128.02 |
| 4034 | OH | TYR | B | 121 | 43.710 | 94.979 | −2.155 | 1.00 | 128.02 |
| 4035 | C | TYR | B | 121 | 40.085 | 91.591 | 4.149 | 1.00 | 134.10 |
| 4036 | O | TYR | B | 121 | 39.556 | 90.483 | 4.039 | 1.00 | 134.10 |
| 4037 | N | LYS | B | 122 | 39.716 | 92.457 | 5.086 | 1.00 | 124.90 |
| 4038 | CA | LYS | B | 122 | 38.635 | 92.165 | 6.013 | 1.00 | 124.90 |
| 4039 | CB | LYS | B | 122 | 39.152 | 92.121 | 7.442 | 1.00 | 161.09 |
| 4040 | CG | LYS | B | 122 | 38.059 | 91.814 | 8.433 | 1.00 | 161.09 |
| 4041 | CD | LYS | B | 122 | 38.511 | 92.092 | 9.842 | 1.00 | 161.09 |
| 4042 | CE | LYS | B | 122 | 37.370 | 91.925 | 10.817 | 1.00 | 161.09 |
| 4043 | NZ | LYS | B | 122 | 37.815 | 92.313 | 12.166 | 1.00 | 161.09 |
| 4044 | C | LYS | B | 122 | 37.606 | 93.289 | 5.874 | 1.00 | 124.90 |
| 4045 | O | LYS | B | 122 | 37.892 | 94.448 | 6.146 | 1.00 | 124.90 |
| 4046 | N | ASP | B | 123 | 36.409 | 92.939 | 5.437 | 1.00 | 160.91 |
| 4047 | CA | ASP | B | 123 | 35.338 | 93.908 | 5.250 | 1.00 | 160.91 |
| 4048 | CB | ASP | B | 123 | 34.877 | 94.474 | 6.591 | 1.00 | 179.88 |
| 4049 | CG | ASP | B | 123 | 34.061 | 93.486 | 7.385 | 1.00 | 179.88 |
| 4050 | OD1 | ASP | B | 123 | 33.105 | 92.913 | 6.817 | 1.00 | 179.88 |
| 4051 | OD2 | ASP | B | 123 | 34.365 | 93.289 | 8.583 | 1.00 | 179.88 |
| 4052 | C | ASP | B | 123 | 35.719 | 95.056 | 4.324 | 1.00 | 160.91 |
| 4053 | O | ASP | B | 123 | 35.556 | 96.222 | 4.671 | 1.00 | 160.91 |
| 4054 | N | GLY | B | 124 | 36.231 | 94.718 | 3.144 | 1.00 | 138.47 |
| 4055 | CA | GLY | B | 124 | 36.604 | 95.728 | 2.160 | 1.00 | 138.47 |
| 4056 | C | GLY | B | 124 | 37.853 | 96.561 | 2.426 | 1.00 | 138.47 |
| 4057 | O | GLY | B | 124 | 38.295 | 97.314 | 1.543 | 1.00 | 138.47 |
| 4058 | N | GLU | B | 125 | 38.430 | 96.420 | 3.621 | 1.00 | 138.22 |
| 4059 | CA | GLU | B | 125 | 39.627 | 97.166 | 4.009 | 1.00 | 138.22 |
| 4060 | CB | GLU | B | 125 | 39.534 | 97.532 | 5.496 | 1.00 | 228.57 |
| 4061 | CG | GLU | B | 125 | 38.461 | 98.543 | 5.849 | 1.00 | 228.57 |
| 4062 | CD | GLU | B | 125 | 38.835 | 99.938 | 5.425 | 1.00 | 228.57 |
| 4063 | OE1 | GLU | B | 125 | 39.838 | 100.460 | 5.952 | 1.00 | 228.57 |
| 4064 | OE2 | GLU | B | 125 | 38.132 | 100.515 | 4.567 | 1.00 | 228.57 |
| 4065 | C | GLU | B | 125 | 40.934 | 96.411 | 3.763 | 1.00 | 138.22 |
| 4066 | O | GLU | B | 125 | 41.001 | 95.195 | 3.933 | 1.00 | 138.22 |
| 4067 | N | ALA | B | 126 | 41.974 | 97.132 | 3.360 | 1.00 | 132.93 |
| 4068 | CA | ALA | B | 126 | 43.266 | 96.495 | 3.155 | 1.00 | 132.93 |
| 4069 | CB | ALA | B | 126 | 44.225 | 97.478 | 2.531 | 1.00 | 135.56 |
| 4070 | C | ALA | B | 126 | 43.730 | 96.115 | 4.563 | 1.00 | 132.93 |
| 4071 | O | ALA | B | 126 | 43.549 | 96.898 | 5.489 | 1.00 | 132.93 |
| 4072 | N | LEU | B | 127 | 44.325 | 94.941 | 4.751 | 1.00 | 121.91 |
| 4073 | CA | LEU | B | 127 | 44.734 | 94.563 | 6.102 | 1.00 | 121.91 |
| 4074 | CB | LEU | B | 127 | 43.939 | 93.352 | 6.540 | 1.00 | 110.11 |
| 4075 | CG | LEU | B | 127 | 43.777 | 93.371 | 8.046 | 1.00 | 110.11 |
| 4076 | CD1 | LEU | B | 127 | 43.167 | 94.684 | 8.464 | 1.00 | 110.11 |
| 4077 | CD2 | LEU | B | 127 | 42.902 | 92.200 | 8.476 | 1.00 | 110.11 |
| 4078 | C | LEU | B | 127 | 46.214 | 94.312 | 6.354 | 1.00 | 121.91 |
| 4079 | O | LEU | B | 127 | 46.818 | 94.960 | 7.211 | 1.00 | 121.91 |
| 4080 | N | LYS | B | 128 | 46.781 | 93.344 | 5.643 | 1.00 | 133.29 |
| 4081 | CA | LYS | B | 128 | 48.199 | 93.025 | 5.779 | 1.00 | 133.29 |
| 4082 | CB | LYS | B | 128 | 48.384 | 91.693 | 6.524 | 1.00 | 231.48 |
| 4083 | CG | LYS | B | 128 | 47.832 | 91.675 | 7.942 | 1.00 | 231.48 |
| 4084 | CD | LYS | B | 128 | 48.657 | 92.524 | 8.899 | 1.00 | 231.48 |
| 4085 | CE | LYS | B | 128 | 48.119 | 92.423 | 10.322 | 1.00 | 231.48 |
| 4086 | NZ | LYS | B | 128 | 49.040 | 93.021 | 11.325 | 1.00 | 231.48 |
| 4087 | C | LYS | B | 128 | 48.770 | 92.918 | 4.365 | 1.00 | 133.29 |
| 4088 | O | LYS | B | 128 | 48.009 | 92.760 | 3.392 | 1.00 | 133.29 |
| 4089 | N | TYR | B | 129 | 50.095 | 92.999 | 4.243 | 1.00 | 155.64 |
| 4090 | CA | TYR | B | 129 | 50.722 | 92.894 | 2.930 | 1.00 | 155.64 |
| 4091 | CB | TYR | B | 129 | 50.745 | 94.261 | 2.262 | 1.00 | 155.17 |
| 4092 | CG | TYR | B | 129 | 51.721 | 94.324 | 1.124 | 1.00 | 155.17 |
| 4093 | CD1 | TYR | B | 129 | 51.372 | 93.892 | −0.149 | 1.00 | 155.17 |
| 4094 | CE1 | TYR | B | 129 | 52.298 | 93.900 | −1.184 | 1.00 | 155.17 |
| 4095 | CD2 | TYR | B | 129 | 53.026 | 94.765 | 1.340 | 1.00 | 155.17 |
| 4096 | CE2 | TYR | B | 129 | 53.965 | 94.775 | 0.315 | 1.00 | 155.17 |
| 4097 | CZ | TYR | B | 129 | 53.593 | 94.340 | −0.950 | 1.00 | 155.17 |
| 4098 | OH | TYR | B | 129 | 54.521 | 94.360 | −1.976 | 1.00 | 155.17 |
| 4099 | C | TYR | B | 129 | 52.144 | 92.321 | 2.965 | 1.00 | 155.64 |
| 4100 | O | TYR | B | 129 | 52.930 | 92.644 | 3.857 | 1.00 | 155.64 |
| 4101 | N | TRP | B | 130 | 52.466 | 91.477 | 1.982 | 1.00 | 137.92 |
| 4102 | CA | TRP | B | 130 | 53.792 | 90.873 | 1.883 | 1.00 | 137.92 |
| 4103 | CB | TRP | B | 130 | 53.871 | 89.553 | 2.659 | 1.00 | 181.18 |
| 4104 | CG | TRP | B | 130 | 53.301 | 89.553 | 4.048 | 1.00 | 181.18 |
| 4105 | CD2 | TRP | B | 130 | 54.032 | 89.540 | 5.275 | 1.00 | 181.18 |
| 4106 | CE2 | TRP | B | 130 | 53.094 | 89.489 | 6.329 | 1.00 | 181.18 |
| 4107 | CE3 | TRP | B | 130 | 55.398 | 89.564 | 5.588 | 1.00 | 181.18 |
| 4108 | CD1 | TRP | B | 130 | 51.980 | 89.516 | 4.397 | 1.00 | 181.18 |
| 4109 | NE1 | TRP | B | 130 | 51.843 | 89.470 | 5.767 | 1.00 | 181.18 |
| 4110 | CZ2 | TRP | B | 130 | 53.470 | 89.458 | 7.667 | 1.00 | 181.18 |
| 4111 | CZ3 | TRP | B | 130 | 55.778 | 89.532 | 6.928 | 1.00 | 181.18 |
| 4112 | CH2 | TRP | B | 130 | 54.816 | 89.490 | 7.949 | 1.00 | 181.18 |
| 4113 | C | TRP | B | 130 | 54.173 | 90.577 | 0.427 | 1.00 | 137.92 |
| 4114 | O | TRP | B | 130 | 53.340 | 90.655 | −0.480 | 1.00 | 137.92 |
| 4115 | N | TYR | B | 131 | 55.439 | 90.218 | 0.220 | 1.00 | 207.31 |
| 4116 | CA | TYR | B | 131 | 55.946 | 89.871 | −1.103 | 1.00 | 207.31 |
| 4117 | CB | TYR | B | 131 | 57.427 | 90.207 | −1.198 | 1.00 | 239.97 |
| 4118 | CG | TYR | B | 131 | 57.952 | 90.151 | −2.603 | 1.00 | 239.97 |
| 4119 | CD1 | TYR | B | 131 | 57.608 | 91.130 | −3.530 | 1.00 | 239.97 |
| 4120 | CE1 | TYR | B | 131 | 58.061 | 91.065 | −4.843 | 1.00 | 239.97 |
| 4121 | CD2 | TYR | B | 131 | 58.768 | 89.101 | −3.020 | 1.00 | 239.97 |
| 4122 | CE2 | TYR | B | 131 | 59.226 | 89.023 | −4.327 | 1.00 | 239.97 |
| 4123 | CZ | TYR | B | 131 | 58.871 | 90.007 | −5.235 | 1.00 | 239.97 |
| 4124 | OH | TYR | B | 131 | 59.314 | 89.922 | −6.534 | 1.00 | 239.97 |
| 4125 | C | TYR | B | 131 | 55.745 | 88.361 | −1.207 | 1.00 | 207.31 |
| 4126 | O | TYR | B | 131 | 54.695 | 87.903 | −1.669 | 1.00 | 207.31 |
| 4127 | N | GLU | B | 132 | 56.760 | 87.590 | −0.805 | 1.00 | 245.95 |
| 4128 | CA | GLU | B | 132 | 56.632 | 86.133 | −0.780 | 1.00 | 245.95 |
| 4129 | CB | GLU | B | 132 | 57.906 | 85.470 | −0.236 | 1.00 | 249.39 |
| 4130 | CG | GLU | B | 132 | 59.071 | 85.349 | −1.220 | 1.00 | 249.39 |
| 4131 | CD | GLU | B | 132 | 59.448 | 83.901 | −1.491 | 1.00 | 249.39 |
| 4132 | OE1 | GLU | B | 132 | 58.976 | 83.011 | −0.751 | 1.00 | 249.39 |
| 4133 | OE2 | GLU | B | 132 | 60.223 | 83.649 | −2.438 | 1.00 | 249.39 |
| 4134 | C | GLU | B | 132 | 55.535 | 86.097 | 0.271 | 1.00 | 245.95 |
| 4135 | O | GLU | B | 132 | 55.697 | 86.700 | 1.341 | 1.00 | 245.95 |
| 4136 | N | ASN | B | 133 | 54.430 | 85.403 | 0.003 | 1.00 | 176.11 |
| 4137 | CA | ASN | B | 133 | 53.339 | 85.462 | 0.965 | 1.00 | 176.11 |
| 4138 | CB | ASN | B | 133 | 52.010 | 84.935 | 0.335 | 1.00 | 160.36 |
| 4139 | CG | ASN | B | 133 | 51.822 | 83.434 | 0.412 | 1.00 | 160.36 |
| 4140 | OD1 | ASN | B | 133 | 52.741 | 82.660 | 0.161 | 1.00 | 160.36 |
| 4141 | ND2 | ASN | B | 133 | 50.589 | 83.015 | 0.716 | 1.00 | 160.36 |
| 4142 | C | ASN | B | 133 | 53.555 | 84.965 | 2.388 | 1.00 | 176.11 |
| 4143 | O | ASN | B | 133 | 54.665 | 84.633 | 2.802 | 1.00 | 176.11 |
| 4144 | N | HIS | B | 134 | 52.479 | 84.997 | 3.151 | 1.00 | 182.29 |
| 4145 | CA | HIS | B | 134 | 52.525 | 84.611 | 4.534 | 1.00 | 182.29 |
| 4146 | CB | HIS | B | 134 | 52.743 | 85.860 | 5.391 | 1.00 | 249.69 |
| 4147 | CG | HIS | B | 134 | 53.003 | 85.559 | 6.845 | 1.00 | 249.69 |
| 4148 | CD2 | HIS | B | 134 | 52.286 | 85.883 | 7.951 | 1.00 | 249.69 |
| 4149 | ND1 | HIS | B | 134 | 54.081 | 84.836 | 7.264 | 1.00 | 249.69 |
| 4150 | CE1 | HIS | B | 134 | 54.036 | 84.709 | 8.595 | 1.00 | 249.69 |
| 4151 | NE2 | HIS | B | 134 | 52.961 | 85.335 | 9.023 | 1.00 | 249.69 |
| 4152 | C | HIS | B | 134 | 51.200 | 83.948 | 4.873 | 1.00 | 182.29 |
| 4153 | O | HIS | B | 134 | 50.446 | 83.556 | 3.984 | 1.00 | 182.29 |
| 4154 | N | ASN | B | 135 | 50.922 | 83.832 | 6.163 | 1.00 | 162.56 |
| 4155 | CA | ASN | B | 135 | 49.712 | 83.206 | 6.644 | 1.00 | 162.56 |
| 4156 | CB | ASN | B | 135 | 49.964 | 81.712 | 6.908 | 1.00 | 240.35 |
| 4157 | CG | ASN | B | 135 | 50.213 | 80.928 | 5.625 | 1.00 | 240.35 |
| 4158 | OD1 | ASN | B | 135 | 49.484 | 81.111 | 4.647 | 1.00 | 240.35 |
| 4159 | ND2 | ASN | B | 135 | 51.216 | 80.048 | 5.620 | 1.00 | 240.35 |
| 4160 | C | ASN | B | 135 | 49.252 | 83.905 | 7.919 | 1.00 | 162.56 |
| 4161 | O | ASN | B | 135 | 49.536 | 83.439 | 9.028 | 1.00 | 162.56 |
| 4162 | N | ILE | B | 136 | 48.544 | 85.024 | 7.745 | 1.00 | 151.20 |
| 4163 | CA | ILE | B | 136 | 47.996 | 85.840 | 8.846 | 1.00 | 151.20 |
| 4164 | CB | ILE | B | 136 | 46.845 | 86.737 | 8.329 | 1.00 | 153.45 |
| 4165 | CG2 | ILE | B | 136 | 45.775 | 85.891 | 7.675 | 1.00 | 153.45 |
| 4166 | CG1 | ILE | B | 136 | 46.230 | 87.526 | 9.477 | 1.00 | 153.45 |
| 4167 | CD1 | ILE | B | 136 | 45.072 | 88.386 | 9.042 | 1.00 | 153.45 |
| 4168 | C | ILE | B | 136 | 47.495 | 85.052 | 10.063 | 1.00 | 151.20 |
| 4169 | O | ILE | B | 136 | 46.403 | 84.466 | 10.041 | 1.00 | 151.20 |
| 4170 | N | SER | B | 137 | 48.291 | 85.084 | 11.133 | 1.00 | 137.33 |
| 4171 | CA | SER | B | 137 | 47.981 | 84.351 | 12.353 | 1.00 | 137.33 |
| 4172 | CB | SER | B | 137 | 49.199 | 83.553 | 12.782 | 1.00 | 185.84 |
| 4173 | OG | SER | B | 137 | 48.986 | 82.999 | 14.062 | 1.00 | 185.84 |
| 4174 | C | SER | B | 137 | 47.481 | 85.154 | 13.549 | 1.00 | 137.33 |
| 4175 | O | SER | B | 137 | 47.862 | 86.294 | 13.764 | 1.00 | 137.33 |
| 4176 | N | ILE | B | 138 | 46.641 | 84.509 | 14.348 | 1.00 | 147.08 |
| 4177 | CA | ILE | B | 138 | 46.043 | 85.114 | 15.529 | 1.00 | 147.08 |
| 4178 | CB | ILE | B | 138 | 44.592 | 85.533 | 15.243 | 1.00 | 140.24 |
| 4179 | CG2 | ILE | B | 138 | 43.867 | 85.830 | 16.544 | 1.00 | 140.24 |
| 4180 | CG1 | ILE | B | 138 | 44.581 | 86.736 | 14.307 | 1.00 | 140.24 |
| 4181 | CD1 | ILE | B | 138 | 43.214 | 87.030 | 13.737 | 1.00 | 140.24 |
| 4182 | C | ILE | B | 138 | 46.047 | 84.165 | 16.723 | 1.00 | 147.08 |
| 4183 | O | ILE | B | 138 | 45.494 | 83.059 | 16.678 | 1.00 | 147.08 |
| 4184 | N | THR | B | 139 | 46.666 | 84.623 | 17.800 | 1.00 | 249.69 |
| 4185 | CA | THR | B | 139 | 46.764 | 83.857 | 19.032 | 1.00 | 249.69 |
| 4186 | CB | THR | B | 139 | 47.944 | 84.369 | 19.853 | 1.00 | 249.31 |
| 4187 | OG1 | THR | B | 139 | 47.808 | 85.783 | 20.034 | 1.00 | 249.31 |
| 4188 | CG2 | THR | B | 139 | 49.251 | 84.099 | 19.122 | 1.00 | 249.31 |
| 4189 | C | THR | B | 139 | 45.473 | 84.015 | 19.832 | 1.00 | 249.69 |
| 4190 | O | THR | B | 139 | 44.591 | 83.158 | 19.795 | 1.00 | 249.69 |
| 4191 | N | ASN | B | 140 | 45.382 | 85.121 | 20.559 | 1.00 | 177.26 |
| 4192 | CA | ASN | B | 140 | 44.211 | 85.439 | 21.351 | 1.00 | 177.26 |
| 4193 | CB | ASN | B | 140 | 44.612 | 86.358 | 22.504 | 1.00 | 249.69 |
| 4194 | CG | ASN | B | 140 | 43.435 | 86.774 | 23.349 | 1.00 | 249.69 |
| 4195 | OD1 | ASN | B | 140 | 42.422 | 87.240 | 22.822 | 1.00 | 249.69 |
| 4196 | ND2 | ASN | B | 140 | 43.566 | 86.631 | 24.665 | 1.00 | 249.69 |
| 4197 | C | ASN | B | 140 | 43.247 | 86.161 | 20.396 | 1.00 | 177.26 |
| 4198 | O | ASN | B | 140 | 43.596 | 87.200 | 19.812 | 1.00 | 177.26 |
| 4199 | N | ALA | B | 141 | 42.040 | 85.615 | 20.236 | 1.00 | 147.92 |
| 4200 | CA | ALA | B | 141 | 41.050 | 86.199 | 19.327 | 1.00 | 147.92 |
| 4201 | CB | ALA | B | 141 | 40.337 | 85.085 | 18.551 | 1.00 | 170.82 |
| 4202 | C | ALA | B | 141 | 40.015 | 87.128 | 19.967 | 1.00 | 147.92 |
| 4203 | O | ALA | B | 141 | 39.333 | 86.783 | 20.942 | 1.00 | 147.92 |
| 4204 | N | THR | B | 142 | 39.910 | 88.316 | 19.389 | 1.00 | 150.18 |
| 4205 | CA | THR | B | 142 | 38.966 | 89.325 | 19.840 | 1.00 | 150.18 |
| 4206 | CB | THR | B | 142 | 39.487 | 90.741 | 19.549 | 1.00 | 194.09 |
| 4207 | OG1 | THR | B | 142 | 40.823 | 90.870 | 20.055 | 1.00 | 194.09 |
| 4208 | CG2 | THR | B | 142 | 38.595 | 91.777 | 20.202 | 1.00 | 194.09 |
| 4209 | C | THR | B | 142 | 37.681 | 89.119 | 19.053 | 1.00 | 150.18 |
| 4210 | O | THR | B | 142 | 37.682 | 88.485 | 17.996 | 1.00 | 150.18 |
| 4211 | N | VAL | B | 143 | 36.580 | 89.650 | 19.560 | 1.00 | 151.70 |
| 4212 | CA | VAL | B | 143 | 35.325 | 89.489 | 18.859 | 1.00 | 151.70 |
| 4213 | CB | VAL | B | 143 | 34.128 | 89.829 | 19.752 | 1.00 | 138.48 |
| 4214 | CG1 | VAL | B | 143 | 34.053 | 91.341 | 19.973 | 1.00 | 138.48 |
| 4215 | CG2 | VAL | B | 143 | 32.853 | 89.308 | 19.122 | 1.00 | 138.48 |
| 4216 | C | VAL | B | 143 | 35.313 | 90.418 | 17.658 | 1.00 | 151.70 |
| 4217 | O | VAL | B | 143 | 34.595 | 90.168 | 16.688 | 1.00 | 151.70 |
| 4218 | N | GLU | B | 144 | 36.106 | 91.488 | 17.716 | 1.00 | 158.91 |
| 4219 | CA | GLU | B | 144 | 36.149 | 92.427 | 16.609 | 1.00 | 158.91 |
| 4220 | CB | GLU | B | 144 | 36.870 | 93.708 | 16.996 | 1.00 | 246.81 |
| 4221 | CG | GLU | B | 144 | 36.208 | 94.445 | 18.127 | 1.00 | 246.81 |
| 4222 | CD | GLU | B | 144 | 37.054 | 94.424 | 19.371 | 1.00 | 246.81 |
| 4223 | OE1 | GLU | B | 144 | 38.183 | 94.958 | 19.317 | 1.00 | 246.81 |
| 4224 | OE2 | GLU | B | 144 | 36.599 | 93.872 | 20.396 | 1.00 | 246.81 |
| 4225 | C | GLU | B | 144 | 36.827 | 91.811 | 15.407 | 1.00 | 158.91 |
| 4226 | O | GLU | B | 144 | 36.756 | 92.346 | 14.312 | 1.00 | 158.91 |
| 4227 | N | ASP | B | 145 | 37.485 | 90.676 | 15.606 | 1.00 | 116.08 |
| 4228 | CA | ASP | B | 145 | 38.161 | 89.992 | 14.501 | 1.00 | 116.08 |
| 4229 | CB | ASP | B | 145 | 39.135 | 88.943 | 15.039 | 1.00 | 169.83 |
| 4230 | CG | ASP | B | 145 | 40.399 | 89.560 | 15.570 | 1.00 | 169.83 |
| 4231 | OD1 | ASP | B | 145 | 41.061 | 90.286 | 14.797 | 1.00 | 169.83 |
| 4232 | OD2 | ASP | B | 145 | 40.728 | 89.327 | 16.756 | 1.00 | 169.83 |
| 4233 | C | ASP | B | 145 | 37.149 | 89.332 | 13.575 | 1.00 | 116.08 |
| 4234 | O | ASP | B | 145 | 37.481 | 88.992 | 12.450 | 1.00 | 116.08 |
| 4235 | N | SER | B | 146 | 35.918 | 89.161 | 14.054 | 1.00 | 118.95 |
| 4236 | CA | SER | B | 146 | 34.858 | 88.538 | 13.267 | 1.00 | 118.95 |
| 4237 | CB | SER | B | 146 | 33.592 | 88.361 | 14.116 | 1.00 | 193.19 |
| 4238 | OG | SER | B | 146 | 33.830 | 87.569 | 15.261 | 1.00 | 193.19 |
| 4239 | C | SER | B | 146 | 34.511 | 89.394 | 12.048 | 1.00 | 118.95 |
| 4240 | O | SER | B | 146 | 34.247 | 90.591 | 12.186 | 1.00 | 118.95 |
| 4241 | N | GLY | B | 147 | 34.487 | 88.787 | 10.863 | 1.00 | 175.20 |
| 4242 | CA | GLY | B | 147 | 34.150 | 89.543 | 9.668 | 1.00 | 175.20 |
| 4243 | C | GLY | B | 147 | 34.222 | 88.715 | 8.406 | 1.00 | 175.20 |
| 4244 | O | GLY | B | 147 | 34.162 | 87.487 | 8.467 | 1.00 | 175.20 |
| 4245 | N | THR | B | 148 | 34.350 | 89.383 | 7.260 | 1.00 | 119.45 |
| 4246 | CA | THR | B | 143 | 34.445 | 88.689 | 5.967 | 1.00 | 119.45 |
| 4247 | CB | THR | B | 148 | 33.224 | 89.059 | 5.057 | 1.00 | 97.36 |
| 4248 | OG1 | THR | B | 148 | 33.606 | 90.066 | 4.118 | 1.00 | 97.36 |
| 4249 | CG2 | THR | B | 148 | 32.069 | 89.581 | 5.907 | 1.00 | 97.36 |
| 4250 | C | THR | B | 148 | 35.802 | 88.969 | 5.256 | 1.00 | 119.45 |
| 4251 | O | THR | B | 148 | 36.102 | 90.077 | 4.838 | 1.00 | 119.45 |
| 4252 | N | TYR | B | 149 | 36.618 | 87.934 | 5.139 | 1.00 | 115.49 |
| 4253 | CA | TYR | B | 149 | 37.918 | 88.061 | 4.528 | 1.00 | 115.49 |
| 4254 | CB | TYR | B | 149 | 38.966 | 87.350 | 5.381 | 1.00 | 121.92 |
| 4255 | CG | TYR | B | 149 | 39.073 | 87.793 | 6.830 | 1.00 | 121.92 |
| 4256 | CD1 | TYR | B | 149 | 38.125 | 87.403 | 7.771 | 1.00 | 121.92 |
| 4257 | CE1 | TYR | B | 149 | 38.255 | 87.752 | 9.102 | 1.00 | 121.92 |
| 4258 | CD2 | TYR | B | 149 | 40.153 | 88.557 | 7.264 | 1.00 | 121.92 |
| 4259 | CE2 | TYR | B | 149 | 40.293 | 88.910 | 8.578 | 1.00 | 121.92 |
| 4260 | CZ | TYR | B | 149 | 39.346 | 88.509 | 9.496 | 1.00 | 121.92 |
| 4261 | OH | TYR | B | 149 | 39.501 | 88.875 | 10.821 | 1.00 | 121.92 |
| 4262 | C | TYR | B | 149 | 38.027 | 87.492 | 3.104 | 1.00 | 115.49 |
| 4263 | O | TYR | B | 149 | 37.182 | 86.708 | 2.670 | 1.00 | 115.49 |
| 4264 | N | TYR | B | 150 | 39.095 | 87.895 | 2.402 | 1.00 | 105.13 |
| 4265 | CA | TYR | B | 150 | 39.437 | 87.467 | 1.042 | 1.00 | 105.13 |
| 4266 | CB | TYR | B | 150 | 38.454 | 88.037 | 0.005 | 1.00 | 148.24 |
| 4267 | CG | TYR | B | 150 | 38.643 | 89.480 | −0.407 | 1.00 | 148.24 |
| 4268 | CD1 | TYR | B | 150 | 39.713 | 89.864 | −1.214 | 1.00 | 148.24 |
| 4269 | CE1 | TYR | B | 150 | 39.871 | 91.204 | −1.645 | 1.00 | 148.24 |
| 4270 | CD2 | TYR | B | 150 | 37.724 | 90.461 | −0.031 | 1.00 | 148.24 |
| 4271 | CE2 | TYR | B | 150 | 37.867 | 91.803 | −0.454 | 1.00 | 148.24 |
| 4272 | CZ | TYR | B | 150 | 38.946 | 92.168 | −1.268 | 1.00 | 148.24 |
| 4273 | OH | TYR | B | 150 | 39.096 | 93.473 | −1.715 | 1.00 | 148.24 |
| 4274 | C | TYR | B | 150 | 40.836 | 88.014 | 0.809 | 1.00 | 105.13 |
| 4275 | O | TYR | B | 150 | 41.228 | 89.015 | 1.414 | 1.00 | 105.13 |
| 4276 | N | CYS | B | 151 | 41.612 | 87.372 | −0.057 | 1.00 | 132.14 |
| 4277 | CA | CYS | B | 151 | 42.986 | 87.840 | −0.332 | 1.00 | 132.14 |
| 4278 | C | CYS | B | 151 | 43.242 | 88.046 | −1.822 | 1.00 | 132.14 |
| 4279 | O | CYS | B | 151 | 42.511 | 87.547 | −2.671 | 1.00 | 132.14 |
| 4280 | CB | CYS | B | 151 | 44.018 | 86.847 | 0.236 | 1.00 | 149.67 |
| 4281 | SG | CYS | B | 151 | 43.926 | 85.150 | −0.462 | 1.00 | 149.67 |
| 4282 | N | THR | B | 152 | 44.288 | 88.798 | −2.129 | 1.00 | 118.50 |
| 4283 | CA | THR | B | 152 | 44.661 | 89.083 | −3.516 | 1.00 | 118.50 |
| 4284 | CB | THR | B | 152 | 44.403 | 90.547 | −3.887 | 1.00 | 151.99 |
| 4285 | OG1 | THR | B | 152 | 45.395 | 91.385 | −3.266 | 1.00 | 151.99 |
| 4286 | CG2 | THR | B | 152 | 43.014 | 90.976 | −3.430 | 1.00 | 151.99 |
| 4287 | C | THR | B | 152 | 46.154 | 88.854 | −3.689 | 1.00 | 118.50 |
| 4288 | O | THR | B | 152 | 46.951 | 89.115 | −2.768 | 1.00 | 118.50 |
| 4289 | N | GLY | B | 153 | 46.544 | 88.378 | −4.864 | 1.00 | 134.78 |
| 4290 | CA | GLY | B | 153 | 47.956 | 88.132 | −5.089 | 1.00 | 134.78 |
| 4291 | C | GLY | B | 153 | 48.309 | 87.906 | −6.545 | 1.00 | 134.78 |
| 4292 | O | GLY | B | 153 | 47.419 | 87.747 | −7.387 | 1.00 | 134.78 |
| 4293 | N | LYS | B | 154 | 49.605 | 87.885 | −6.842 | 1.00 | 183.95 |
| 4294 | CA | LYS | B | 154 | 50.077 | 87.673 | −8.199 | 1.00 | 183.95 |
| 4295 | CB | LYS | B | 154 | 51.115 | 88.730 | −8.542 | 1.00 | 202.68 |
| 4296 | CG | LYS | B | 154 | 51.611 | 88.679 | −9.971 | 1.00 | 202.68 |
| 4297 | CD | LYS | B | 154 | 52.591 | 89.815 | −10.237 | 1.00 | 202.68 |
| 4298 | CE | LYS | B | 154 | 53.158 | 89.756 | −11.649 | 1.00 | 202.68 |
| 4299 | NZ | LYS | B | 154 | 54.131 | 90.860 | −11.921 | 1.00 | 202.68 |
| 4300 | C | LYS | B | 154 | 50.672 | 86.268 | −8.377 | 1.00 | 183.95 |
| 4301 | O | LYS | B | 154 | 51.657 | 85.900 | −7.729 | 1.00 | 183.95 |
| 4302 | N | VAL | B | 155 | 50.048 | 85.484 | −9.253 | 1.00 | 214.72 |
| 4303 | CA | VAL | B | 155 | 50.480 | 84.117 | −9.558 | 1.00 | 214.72 |
| 4304 | CB | VAL | B | 155 | 49.275 | 83.155 | −9.620 | 1.00 | 178.34 |
| 4305 | CG1 | VAL | B | 155 | 49.717 | 81.766 | −10.018 | 1.00 | 178.34 |
| 4306 | CG2 | VAL | B | 155 | 48.581 | 83.118 | −8.276 | 1.00 | 178.34 |
| 4307 | C | VAL | B | 155 | 51.122 | 84.193 | −10.931 | 1.00 | 214.72 |
| 4308 | O | VAL | B | 155 | 50.530 | 84.738 | −11.865 | 1.00 | 214.72 |
| 4309 | N | TRP | B | 156 | 52.321 | 83.636 | −11.061 | 1.00 | 210.74 |
| 4310 | CA | TRP | B | 156 | 53.050 | 83.695 | −12.334 | 1.00 | 210.74 |
| 4311 | CB | TRP | B | 156 | 52.245 | 83.105 | −13.514 | 1.00 | 249.69 |
| 4312 | CG | TRP | B | 156 | 51.997 | 81.615 | −13.504 | 1.00 | 249.69 |
| 4313 | CD2 | TRP | B | 156 | 52.972 | 80.576 | −13.670 | 1.00 | 249.69 |
| 4314 | CE2 | TRP | B | 156 | 52.277 | 79.346 | −13.606 | 1.00 | 249.69 |
| 4315 | CE3 | TRP | B | 156 | 54.358 | 80.563 | −13.864 | 1.00 | 249.69 |
| 4316 | CD1 | TRP | B | 156 | 50.790 | 80.988 | −13.351 | 1.00 | 249.69 |
| 4317 | NE1 | TRP | B | 156 | 50.950 | 79.628 | −13.414 | 1.00 | 249.69 |
| 4318 | CZ2 | TRP | B | 156 | 52.925 | 78.114 | −13.729 | 1.00 | 249.69 |
| 4319 | CZ3 | TRP | B | 156 | 55.003 | 79.336 | −13.990 | 1.00 | 249.69 |
| 4320 | CH2 | TRP | B | 156 | 54.285 | 78.129 | −13.915 | 1.00 | 249.69 |
| 4321 | C | TRP | B | 156 | 53.249 | 85.183 | −12.595 | 1.00 | 210.74 |
| 4322 | O | TRP | B | 156 | 54.162 | 85.808 | −12.059 | 1.00 | 210.74 |
| 4323 | N | GLN | B | 157 | 52.355 | 85.739 | −13.408 | 1.00 | 156.28 |
| 4324 | CA | GLN | B | 157 | 52.406 | 87.144 | −13.767 | 1.00 | 156.28 |
| 4325 | CB | GLN | B | 157 | 53.203 | 87.338 | −15.074 | 1.00 | 249.69 |
| 4326 | CG | GLN | B | 157 | 54.719 | 87.195 | −14.910 | 1.00 | 249.69 |
| 4327 | CD | GLN | B | 157 | 55.327 | 88.274 | −14.019 | 1.00 | 249.69 |
| 4328 | OE1 | GLN | B | 157 | 55.277 | 89.465 | −14.340 | 1.00 | 249.69 |
| 4329 | NE2 | GLN | B | 157 | 55.906 | 87.860 | −12.893 | 1.00 | 249.69 |
| 4330 | C | GLN | B | 157 | 51.035 | 87.808 | −13.891 | 1.00 | 156.28 |
| 4331 | O | GLN | B | 157 | 50.893 | 88.830 | −14.565 | 1.00 | 156.28 |
| 4332 | N | LEU | B | 158 | 50.028 | 87.237 | −13.243 | 1.00 | 204.79 |
| 4333 | CA | LEU | B | 158 | 48.698 | 87.836 | −13.267 | 1.00 | 204.79 |
| 4334 | CB | LEU | B | 158 | 47.745 | 87.025 | −14.156 | 1.00 | 162.40 |
| 4335 | CG | LEU | B | 158 | 47.849 | 87.176 | −15.669 | 1.00 | 162.40 |
| 4336 | CD1 | LEU | B | 158 | 46.441 | 87.082 | −16.258 | 1.00 | 162.40 |
| 4337 | CD2 | LEU | B | 158 | 48.464 | 88.518 | −16.037 | 1.00 | 162.40 |
| 4338 | C | LEU | B | 158 | 48.124 | 87.959 | −11.852 | 1.00 | 204.79 |
| 4339 | O | LEU | B | 158 | 48.450 | 87.172 | −10.964 | 1.00 | 204.79 |
| 4340 | N | ASP | B | 159 | 47.277 | 88.961 | −11.648 | 1.00 | 211.13 |
| 4341 | CA | ASP | B | 159 | 46.669 | 89.179 | −10.351 | 1.00 | 211.13 |
| 4342 | CB | ASP | B | 159 | 46.316 | 90.659 | −10.178 | 1.00 | 203.85 |
| 4343 | CG | ASP | B | 159 | 47.491 | 91.574 | −10.416 | 1.00 | 203.85 |
| 4344 | OD1 | ASP | B | 159 | 48.489 | 91.461 | −9.680 | 1.00 | 203.85 |
| 4345 | OD2 | ASP | B | 159 | 47.413 | 92.407 | −11.342 | 1.00 | 203.85 |
| 4346 | C | ASP | B | 159 | 45.397 | 88.344 | −10.208 | 1.00 | 211.13 |
| 4347 | O | ASP | B | 159 | 44.695 | 88.096 | −11.193 | 1.00 | 211.13 |
| 4348 | N | TYR | B | 160 | 45.101 | 87.907 | −8.984 | 1.00 | 157.46 |
| 4349 | CA | TYR | B | 160 | 43.891 | 87.129 | −8.729 | 1.00 | 157.46 |
| 4350 | CB | TYR | B | 160 | 44.174 | 85.638 | −8.836 | 1.00 | 182.37 |
| 4351 | CG | TYR | B | 160 | 44.781 | 85.232 | −10.147 | 1.00 | 182.37 |
| 4352 | CD1 | TYR | B | 160 | 46.161 | 85.213 | −10.315 | 1.00 | 182.37 |
| 4353 | CE1 | TYR | B | 160 | 46.733 | 84.810 | −11.517 | 1.00 | 182.37 |
| 4354 | CD2 | TYR | B | 160 | 43.977 | 84.849 | −11.217 | 1.00 | 182.37 |
| 4355 | CE2 | TYR | B | 160 | 44.537 | 84.446 | −12.433 | 1.00 | 182.37 |
| 4356 | CZ | TYR | B | 160 | 45.917 | 84.423 | −12.578 | 1.00 | 182.37 |
| 4357 | OH | TYR | B | 160 | 46.481 | 83.987 | −13.765 | 1.00 | 182.37 |
| 4358 | C | TYR | B | 160 | 43.275 | 87.423 | −7.372 | 1.00 | 157.46 |
| 4359 | O | TYR | B | 160 | 43.977 | 87.686 | −6.377 | 1.00 | 157.46 |
| 4360 | N | GLU | B | 161 | 41.947 | 87.358 | −7.357 | 1.00 | 159.09 |
| 4361 | CA | GLU | B | 161 | 41.161 | 87.625 | −6.168 | 1.00 | 159.09 |
| 4362 | CB | GLU | B | 161 | 40.085 | 88.664 | −6.527 | 1.00 | 208.73 |
| 4363 | CG | GLU | B | 161 | 39.125 | 89.081 | −5.420 | 1.00 | 208.73 |
| 4364 | CD | GLU | B | 161 | 38.358 | 90.353 | −5.769 | 1.00 | 208.73 |
| 4365 | OE1 | GLU | B | 161 | 37.289 | 90.587 | −5.169 | 1.00 | 208.73 |
| 4366 | OE2 | GLU | B | 161 | 38.829 | 91.125 | −6.633 | 1.00 | 208.73 |
| 4367 | C | GLU | B | 161 | 40.553 | 86.311 | −5.703 | 1.00 | 159.09 |
| 4368 | O | GLU | B | 161 | 40.167 | 85.483 | −6.531 | 1.00 | 159.09 |
| 4369 | N | SER | B | 162 | 40.491 | 86.120 | −4.386 | 1.00 | 135.59 |
| 4370 | CA | SER | B | 162 | 39.945 | 84.894 | −3.798 | 1.00 | 135.59 |
| 4371 | CB | SER | B | 162 | 40.698 | 84.552 | −2.508 | 1.00 | 156.53 |
| 4372 | OG | SER | B | 162 | 40.507 | 85.561 | −1.523 | 1.00 | 156.53 |
| 4373 | C | SER | B | 162 | 38.467 | 85.022 | −3.475 | 1.00 | 135.59 |
| 4374 | O | SER | B | 162 | 37.948 | 86.125 | −3.362 | 1.00 | 135.59 |
| 4375 | N | GLU | B | 163 | 37.785 | 83.891 | −3.332 | 1.00 | 183.98 |
| 4376 | CA | GLU | B | 163 | 36.382 | 83.933 | −2.974 | 1.00 | 183.98 |
| 4377 | CB | GLU | B | 163 | 35.794 | 82.514 | −2.941 | 1.00 | 249.53 |
| 4378 | CG | GLU | B | 163 | 35.543 | 81.880 | −4.311 | 1.00 | 249.53 |
| 4379 | CD | GLU | B | 163 | 34.339 | 82.475 | −5.030 | 1.00 | 249.53 |
| 4380 | OE1 | GLU | B | 163 | 33.229 | 82.464 | −4.456 | 1.00 | 249.53 |
| 4381 | OE2 | GLU | B | 163 | 34.498 | 82.945 | −6.173 | 1.00 | 249.53 |
| 4382 | C | GLU | B | 163 | 36.369 | 84.544 | −1.569 | 1.00 | 183.98 |
| 4383 | O | GLU | B | 163 | 37.334 | 84.379 | −0.818 | 1.00 | 183.98 |
| 4384 | N | PRO | B | 164 | 35.304 | 85.273 | −1.199 | 1.00 | 111.61 |
| 4385 | CD | PRO | B | 164 | 34.138 | 85.647 | −2.030 | 1.00 | 194.84 |
| 4386 | CA | PRO | B | 164 | 35.206 | 85.903 | 0.122 | 1.00 | 111.61 |
| 4387 | CB | PRO | B | 164 | 34.171 | 86.986 | −0.090 | 1.00 | 194.84 |
| 4388 | CG | PRO | B | 164 | 33.217 | 86.310 | −1.015 | 1.00 | 194.84 |
| 4389 | C | PRO | B | 164 | 34.778 | 84.904 | 1.197 | 1.00 | 111.61 |
| 4390 | O | PRO | B | 164 | 33.994 | 83.997 | 0.926 | 1.00 | 111.61 |
| 4391 | N | LEU | B | 165 | 35.268 | 85.078 | 2.417 | 1.00 | 125.70 |
| 4392 | CA | LEU | B | 165 | 34.918 | 84.150 | 3.482 | 1.00 | 125.70 |
| 4393 | CB | LEU | B | 165 | 36.104 | 83.241 | 3.771 | 1.00 | 110.19 |
| 4394 | CG | LEU | B | 165 | 35.906 | 82.190 | 4.868 | 1.00 | 110.19 |
| 4395 | CD1 | LEU | B | 165 | 34.494 | 81.640 | 4.805 | 1.00 | 110.19 |
| 4396 | CD2 | LEU | B | 165 | 36.944 | 81.076 | 4.711 | 1.00 | 110.19 |
| 4397 | C | LEU | B | 165 | 34.468 | 84.800 | 4.775 | 1.00 | 125.70 |
| 4398 | O | LEU | B | 165 | 35.127 | 85.716 | 5.274 | 1.00 | 125.70 |
| 4399 | N | ASN | B | 166 | 33.348 | 84.317 | 5.319 | 1.00 | 120.14 |
| 4400 | CA | ASN | B | 166 | 32.809 | 84.855 | 6.577 | 1.00 | 120.14 |
| 4401 | CB | ASN | B | 166 | 31.283 | 84.695 | 6.635 | 1.00 | 189.75 |
| 4402 | CG | ASN | B | 166 | 30.537 | 85.923 | 6.141 | 1.00 | 189.75 |
| 4403 | OD1 | ASN | B | 166 | 31.053 | 87.039 | 6.184 | 1.00 | 189.75 |
| 4404 | ND2 | ASN | B | 166 | 29.303 | 85.713 | 5.694 | 1.00 | 189.75 |
| 4405 | C | ASN | B | 166 | 33.420 | 84.164 | 7.793 | 1.00 | 120.14 |
| 4406 | O | ASN | B | 166 | 33.718 | 82.978 | 7.752 | 1.00 | 120.14 |
| 4407 | N | ILE | B | 167 | 33.594 | 84.911 | 8.875 | 1.00 | 133.16 |
| 4408 | CA | ILE | B | 167 | 34.163 | 84.359 | 10.087 | 1.00 | 133.16 |
| 4409 | CB | ILE | B | 167 | 35.634 | 84.660 | 10.178 | 1.00 | 105.73 |
| 4410 | CG2 | ILE | B | 167 | 36.159 | 84.179 | 11.513 | 1.00 | 105.73 |
| 4411 | CG1 | ILE | B | 167 | 36.355 | 83.972 | 9.038 | 1.00 | 105.73 |
| 4412 | CD1 | ILE | B | 167 | 37.820 | 84.256 | 9.039 | 1.00 | 105.73 |
| 4413 | C | ILE | B | 167 | 33.510 | 84.926 | 11.319 | 1.00 | 133.16 |
| 4414 | O | ILE | B | 167 | 33.451 | 86.140 | 11.480 | 1.00 | 133.16 |
| 4415 | N | THR | B | 168 | 33.058 | 84.057 | 12.213 | 1.00 | 113.92 |
| 4416 | CA | THR | B | 168 | 32.409 | 84.557 | 13.402 | 1.00 | 113.92 |
| 4417 | CB | THR | B | 168 | 30.931 | 84.189 | 13.377 | 1.00 | 138.53 |
| 4418 | OG1 | THR | B | 168 | 30.347 | 84.678 | 12.163 | 1.00 | 138.53 |
| 4419 | CG2 | THR | B | 168 | 30.214 | 84.810 | 14.563 | 1.00 | 138.53 |
| 4420 | C | THR | B | 168 | 33.018 | 84.135 | 14.728 | 1.00 | 113.92 |
| 4421 | O | THR | B | 168 | 33.161 | 82.955 | 15.019 | 1.00 | 113.92 |
| 4422 | N | VAL | B | 169 | 33.381 | 85.129 | 15.526 | 1.00 | 134.74 |
| 4423 | CA | VAL | B | 169 | 33.953 | 84.905 | 16.840 | 1.00 | 134.74 |
| 4424 | CB | VAL | B | 169 | 35.207 | 85.795 | 17.049 | 1.00 | 119.12 |
| 4425 | CG1 | VAL | B | 169 | 35.482 | 85.994 | 18.518 | 1.00 | 119.12 |
| 4426 | CG2 | VAL | B | 169 | 36.403 | 85.141 | 16.397 | 1.00 | 119.12 |
| 4427 | C | VAL | B | 169 | 32.864 | 85.249 | 17.868 | 1.00 | 134.74 |
| 4428 | O | VAL | B | 169 | 32.511 | 86.415 | 18.047 | 1.00 | 134.74 |
| 4429 | N | ILE | B | 170 | 32.326 | 84.222 | 18.527 | 1.00 | 162.52 |
| 4430 | CA | ILE | B | 170 | 31.270 | 84.383 | 19.532 | 1.00 | 162.52 |
| 4431 | CB | ILE | B | 170 | 30.271 | 83.207 | 19.449 | 1.00 | 162.30 |
| 4432 | CG2 | ILE | B | 170 | 29.809 | 83.021 | 18.010 | 1.00 | 162.30 |
| 4433 | CG1 | ILE | B | 170 | 30.946 | 81.906 | 19.881 | 1.00 | 162.30 |
| 4434 | CD1 | ILE | B | 170 | 30.029 | 80.693 | 19.859 | 1.00 | 162.30 |
| 4435 | C | ILE | B | 170 | 31.881 | 84.444 | 20.932 | 1.00 | 162.52 |
| 4436 | O | ILE | B | 170 | 33.039 | 84.092 | 21.122 | 1.00 | 162.52 |
| 4437 | N | LYS | B | 171 | 31.114 | 84.869 | 21.923 | 1.00 | 183.47 |
| 4438 | CA | LYS | B | 171 | 31.659 | 84.966 | 23.278 | 1.00 | 183.47 |
| 4439 | CB | LYS | B | 171 | 31.632 | 86.425 | 23.716 | 1.00 | 228.46 |
| 4440 | CG | LYS | B | 171 | 30.255 | 87.040 | 23.602 | 1.00 | 228.46 |
| 4441 | CD | LYS | B | 171 | 30.316 | 88.539 | 23.398 | 1.00 | 228.46 |
| 4442 | CE | LYS | B | 171 | 31.046 | 89.232 | 24.538 | 1.00 | 228.46 |
| 4443 | NZ | LYS | B | 171 | 31.040 | 90.714 | 24.377 | 1.00 | 228.46 |
| 4444 | C | LYS | B | 171 | 30.941 | 84.101 | 24.319 | 1.00 | 183.47 |
| 4445 | O | LYS | B | 171 | 31.193 | 84.218 | 25.525 | 1.00 | 183.47 |
| 4446 | C1 | NAG | B | 221 | 48.145 | 62.916 | −2.146 | 1.00 | 249.69 |
| 4447 | C2 | NAG | B | 221 | 49.283 | 63.891 | −2.430 | 1.00 | 249.69 |
| 4448 | N2 | NAG | B | 221 | 48.728 | 65.201 | −2.707 | 1.00 | 249.69 |
| 4449 | C7 | NAG | B | 221 | 49.464 | 66.290 | −2.515 | 1.00 | 249.69 |
| 4450 | O7 | NAG | B | 221 | 50.628 | 66.249 | −2.115 | 1.00 | 249.69 |
| 4451 | C8 | NAG | B | 221 | 48.813 | 67.630 | −2.819 | 1.00 | 249.69 |
| 4452 | C3 | NAG | B | 221 | 50.117 | 63.412 | −3.614 | 1.00 | 249.69 |
| 4453 | O3 | NAG | B | 221 | 51.258 | 64.240 | −3.757 | 1.00 | 249.69 |
| 4454 | C4 | NAG | B | 221 | 50.568 | 61.956 | −3.448 | 1.00 | 249.69 |
| 4455 | O4 | NAG | B | 221 | 51.118 | 61.532 | −4.713 | 1.00 | 249.69 |
| 4456 | C5 | NAG | B | 221 | 49.362 | 61.063 | −3.063 | 1.00 | 249.69 |
| 4457 | O5 | NAG | B | 221 | 48.675 | 61.604 | −1.912 | 1.00 | 249.69 |
| 4458 | C6 | NAG | B | 221 | 49.751 | 59.637 | −2.698 | 1.00 | 249.69 |
| 4459 | O6 | NAG | B | 221 | 50.700 | 59.612 | −1.642 | 1.00 | 249.69 |
| 4460 | C1 | NAG | B | 222 | 51.927 | 60.395 | −4.782 | 1.00 | 249.69 |
| 4461 | C2 | NAG | B | 222 | 53.144 | 60.683 | −5.692 | 1.00 | 249.69 |
| 4462 | N2 | NAG | B | 222 | 53.932 | 61.775 | −5.134 | 1.00 | 249.69 |
| 4463 | C7 | NAG | B | 222 | 55.211 | 61.597 | −4.802 | 1.00 | 249.69 |
| 4464 | O7 | NAG | B | 222 | 55.800 | 60.524 | −4.944 | 1.00 | 249.69 |
| 4465 | C8 | NAG | B | 222 | 55.954 | 62.796 | −4.227 | 1.00 | 249.69 |
| 4466 | C3 | NAG | B | 222 | 52.654 | 61.043 | −7.123 | 1.00 | 249.69 |
| 4467 | O3 | NAG | B | 222 | 53.764 | 61.185 | −8.006 | 1.00 | 249.69 |
| 4468 | C4 | NAG | B | 222 | 51.697 | 59.961 | −7.668 | 1.00 | 249.69 |
| 4469 | O4 | NAG | B | 222 | 51.134 | 60.393 | −8.900 | 1.00 | 249.69 |
| 4470 | C5 | NAG | B | 222 | 50.571 | 59.682 | −6.652 | 1.00 | 249.69 |
| 4471 | O5 | NAG | B | 222 | 51.140 | 59.328 | −5.356 | 1.00 | 249.69 |
| 4472 | C6 | NAG | B | 222 | 49.642 | 58.551 | −7.073 | 1.00 | 249.69 |
| 4473 | O6 | NAG | B | 222 | 48.276 | 58.935 | −6.979 | 1.00 | 249.69 |
| 4474 | C1 | NAG | B | 242 | 27.093 | 65.374 | −0.289 | 1.00 | 220.33 |
| 4475 | C2 | NAG | B | 242 | 27.619 | 64.790 | −1.606 | 1.00 | 220.33 |
| 4476 | N2 | NAG | B | 242 | 28.439 | 63.617 | −1.372 | 1.00 | 220.33 |
| 4477 | C7 | NAG | B | 242 | 28.079 | 62.446 | −1.890 | 1.00 | 220.33 |
| 4478 | O7 | NAG | B | 242 | 27.061 | 62.304 | −2.564 | 1.00 | 220.33 |
| 4479 | C8 | NAG | B | 242 | 28.977 | 61.252 | −1.616 | 1.00 | 220.33 |
| 4480 | C3 | NAG | B | 242 | 28.417 | 65.869 | −2.342 | 1.00 | 220.33 |
| 4481 | O3 | NAG | B | 242 | 28.893 | 65.358 | −3.579 | 1.00 | 220.33 |
| 4482 | C4 | NAG | B | 242 | 27.524 | 67.092 | −2.588 | 1.00 | 220.33 |
| 4483 | O4 | NAG | B | 242 | 28.320 | 68.165 | −3.127 | 1.00 | 220.33 |
| 4484 | C5 | NAG | B | 242 | 26.849 | 67.565 | −1.278 | 1.00 | 220.33 |
| 4485 | O5 | NAG | B | 242 | 26.201 | 66.460 | −0.583 | 1.00 | 220.33 |
| 4486 | C6 | NAG | B | 242 | 25.764 | 68.596 | −1.552 | 1.00 | 220.33 |
| 4487 | O6 | NAG | B | 242 | 26.133 | 69.886 | −1.090 | 1.00 | 220.33 |
| 4488 | C1 | NAG | B | 243 | 27.960 | 68.648 | −4.371 | 1.00 | 233.97 |
| 4489 | C2 | NAG | B | 243 | 28.552 | 70.043 | −4.570 | 1.00 | 233.97 |
| 4490 | N2 | NAG | B | 243 | 28.067 | 70.964 | −3.561 | 1.00 | 233.97 |
| 4491 | C7 | NAG | B | 243 | 28.929 | 71.745 | −2.911 | 1.00 | 233.97 |
| 4492 | O7 | NAG | B | 243 | 30.147 | 71.719 | −3.114 | 1.00 | 233.97 |
| 4493 | C8 | NAG | B | 243 | 28.358 | 72.696 | −1.871 | 1.00 | 233.97 |
| 4494 | C3 | NAG | B | 243 | 28.185 | 70.544 | −5.960 | 1.00 | 233.97 |
| 4495 | O3 | NAG | B | 243 | 28.726 | 71.840 | −6.174 | 1.00 | 233.97 |
| 4496 | C4 | NAG | B | 243 | 28.751 | 69.586 | −6.984 | 1.00 | 233.97 |
| 4497 | O4 | NAG | B | 243 | 28.443 | 70.118 | −8.263 | 1.00 | 233.97 |
| 4498 | C5 | NAG | B | 243 | 28.175 | 68.165 | −6.727 | 1.00 | 233.97 |
| 4499 | O5 | NAG | B | 243 | 28.488 | 67.756 | −5.361 | 1.00 | 233.97 |
| 4500 | C6 | NAG | B | 243 | 28.776 | 67.113 | −7.637 | 1.00 | 233.97 |
| 4501 | O6 | NAG | B | 243 | 30.175 | 66.991 | −7.430 | 1.00 | 233.97 |
| 4502 | C1 | MAN | B | 244 | 29.240 | 69.921 | −9.345 | 1.00 | 229.91 |
| 4503 | C2 | MAN | B | 244 | 28.260 | 69.705 | −10.400 | 1.00 | 229.91 |
| 4504 | O2 | MAN | B | 244 | 27.196 | 70.659 | −10.238 | 1.00 | 229.91 |
| 4505 | C3 | MAN | B | 244 | 28.928 | 69.691 | −11.752 | 1.00 | 229.91 |
| 4506 | O3 | MAN | B | 244 | 28.001 | 69.355 | −12.770 | 1.00 | 229.91 |
| 4507 | C4 | MAN | B | 244 | 29.658 | 70.989 | −12.013 | 1.00 | 229.91 |
| 4508 | O4 | MAN | B | 244 | 30.237 | 70.964 | −13.307 | 1.00 | 229.91 |
| 4509 | C5 | MAN | B | 244 | 30.732 | 71.128 | −10.933 | 1.00 | 229.91 |
| 4510 | O5 | MAN | B | 244 | 30.062 | 71.170 | −9.601 | 1.00 | 229.91 |
| 4511 | C6 | MAN | B | 244 | 31.699 | 72.322 | −11.158 | 1.00 | 229.91 |
| 4512 | O6 | MAN | B | 244 | 31.180 | 73.559 | −10.690 | 1.00 | 229.91 |
| 4513 | C1 | NAG | B | 250 | 44.268 | 53.492 | 9.707 | 1.00 | 249.69 |
| 4514 | C2 | NAG | B | 250 | 45.671 | 53.603 | 10.328 | 1.00 | 249.69 |
| 4515 | N2 | NAG | B | 250 | 45.573 | 53.779 | 11.763 | 1.00 | 249.69 |
| 4516 | C7 | NAG | B | 250 | 45.937 | 52.792 | 12.578 | 1.00 | 249.69 |
| 4517 | O7 | NAG | B | 250 | 46.363 | 51.706 | 12.172 | 1.00 | 249.69 |
| 4518 | C8 | NAG | B | 250 | 45.811 | 53.044 | 14.074 | 1.00 | 249.69 |
| 4519 | C3 | NAG | B | 250 | 46.415 | 54.790 | 9.702 | 1.00 | 249.69 |
| 4520 | O3 | NAG | B | 250 | 47.749 | 54.843 | 10.194 | 1.00 | 249.69 |
| 4521 | C4 | NAG | B | 250 | 46.432 | 54.657 | 8.172 | 1.00 | 249.69 |
| 4522 | O4 | NAG | B | 250 | 47.008 | 55.826 | 7.602 | 1.00 | 249.69 |
| 4523 | C5 | NAG | B | 250 | 44.994 | 54.460 | 7.640 | 1.00 | 249.69 |
| 4524 | O5 | NAG | B | 250 | 44.369 | 53.324 | 8.287 | 1.00 | 249.69 |
| 4525 | C6 | NAG | B | 250 | 44.929 | 54.206 | 6.139 | 1.00 | 249.69 |
| 4526 | O6 | NAG | B | 250 | 43.668 | 53.664 | 5.761 | 1.00 | 249.69 |
| 4527 | C1 | NAG | B | 274 | 23.582 | 59.809 | 24.027 | 1.00 | 249.69 |
| 4528 | O2 | NAG | B | 274 | 23.459 | 61.065 | 24.903 | 1.00 | 249.69 |
| 4529 | N2 | NAG | B | 274 | 24.613 | 61.181 | 25.777 | 1.00 | 249.69 |
| 4530 | C7 | NAG | B | 274 | 24.999 | 62.374 | 26.223 | 1.00 | 249.69 |
| 4531 | O7 | NAG | B | 274 | 24.418 | 63.422 | 25.934 | 1.00 | 249.69 |
| 4532 | O8 | NAG | B | 274 | 26.218 | 62.415 | 27.133 | 1.00 | 249.69 |
| 4533 | C3 | NAG | B | 274 | 22.167 | 60.997 | 25.741 | 1.00 | 249.69 |
| 4534 | O3 | NAG | B | 274 | 21.983 | 62.216 | 26.451 | 1.00 | 249.69 |
| 4535 | C4 | NAG | B | 274 | 20.951 | 60.745 | 24.836 | 1.00 | 249.69 |
| 4536 | O4 | NAG | B | 274 | 19.788 | 60.553 | 25.637 | 1.00 | 249.69 |
| 4537 | C5 | NAG | B | 274 | 21.198 | 59.506 | 23.958 | 1.00 | 249.69 |
| 4538 | O5 | NAG | B | 274 | 22.418 | 59.674 | 23.192 | 1.00 | 249.69 |
| 4539 | C6 | NAG | B | 274 | 20.073 | 59.255 | 22.962 | 1.00 | 249.69 |
| 4540 | O6 | NAG | B | 274 | 20.404 | 58.209 | 22.054 | 1.00 | 249.69 |
| 4541 | O1 | NAG | B | 335 | 50.936 | 78.660 | 5.286 | 1.00 | 249.69 |
| 4542 | C2 | NAG | B | 335 | 51.372 | 77.658 | 6.389 | 1.00 | 249.69 |
| 4543 | N2 | NAG | B | 335 | 51.470 | 78.372 | 7.651 | 1.00 | 249.69 |
| 4544 | C7 | NAG | B | 335 | 50.669 | 78.063 | 8.668 | 1.00 | 249.69 |
| 4545 | O7 | NAG | B | 335 | 49.823 | 77.166 | 8.611 | 1.00 | 249.69 |
| 4546 | C8 | NAG | B | 335 | 50.837 | 78.865 | 9.950 | 1.00 | 249.69 |
| 4547 | C3 | NAG | B | 335 | 52.711 | 76.938 | 6.115 | 1.00 | 249.69 |
| 4548 | O3 | NAG | B | 335 | 52.790 | 75.759 | 6.909 | 1.00 | 249.69 |
| 4549 | C4 | NAG | B | 335 | 52.852 | 76.553 | 4.647 | 1.00 | 249.69 |
| 4550 | O4 | NAG | B | 335 | 54.131 | 75.970 | 4.410 | 1.00 | 249.69 |
| 4551 | C5 | NAG | B | 335 | 52.678 | 77.814 | 3.812 | 1.00 | 249.69 |
| 4552 | O5 | NAG | B | 335 | 51.319 | 78.289 | 3.939 | 1.00 | 249.69 |
| 4553 | C6 | NAG | B | 335 | 52.935 | 77.564 | 2.334 | 1.00 | 249.69 |
| 4554 | O6 | NAG | B | 335 | 53.923 | 78.447 | 1.826 | 1.00 | 249.69 |
| 4555 | C1 | NAG | B | 340 | 43.529 | 87.808 | 25.515 | 1.00 | 249.69 |
| 4556 | C2 | NAG | B | 340 | 42.252 | 87.842 | 26.379 | 1.00 | 249.69 |
| 4557 | N2 | NAG | B | 340 | 41.073 | 87.751 | 25.533 | 1.00 | 249.69 |
| 4558 | C7 | NAG | B | 340 | 40.086 | 86.909 | 25.835 | 1.00 | 249.69 |
| 4559 | O7 | NAG | B | 340 | 40.099 | 86.177 | 26.833 | 1.00 | 249.69 |
| 4560 | C8 | NAG | B | 340 | 38.898 | 86.878 | 24.882 | 1.00 | 249.69 |
| 4561 | C3 | NAG | B | 340 | 42.235 | 89.153 | 27.182 | 1.00 | 249.69 |
| 4562 | O3 | NAG | B | 340 | 41.117 | 89.172 | 28.061 | 1.00 | 249.69 |
| 4563 | C4 | NAG | B | 340 | 43.537 | 89.304 | 27.991 | 1.00 | 249.69 |
| 4564 | O4 | NAG | B | 340 | 43.566 | 90.587 | 28.606 | 1.00 | 249.69 |
| 4565 | C5 | NAG | B | 340 | 44.768 | 89.134 | 27.069 | 1.00 | 249.69 |
| 4566 | O5 | NAG | B | 340 | 44.691 | 87.877 | 26.352 | 1.00 | 249.69 |
| 4567 | C6 | NAG | B | 340 | 46.101 | 89.143 | 27.805 | 1.00 | 249.69 |
| 4568 | O6 | NAG | B | 340 | 47.172 | 88.783 | 26.936 | 1.00 | 249.69 |
| 4569 | C1 | NAG | B | 366 | 28.566 | 86.792 | 5.084 | 1.00 | 212.59 |
| 4570 | C2 | NAG | B | 366 | 27.738 | 86.264 | 3.928 | 1.00 | 212.59 |
| 4571 | N2 | NAG | B | 366 | 28.623 | 85.657 | 2.952 | 1.00 | 212.59 |
| 4572 | C7 | NAG | B | 366 | 28.903 | 84.360 | 3.019 | 1.00 | 212.59 |
| 4573 | O7 | NAG | B | 366 | 28.430 | 83.623 | 3.883 | 1.00 | 212.59 |
| 4574 | C8 | NAG | B | 366 | 29.845 | 83.792 | 1.966 | 1.00 | 212.59 |
| 4575 | C3 | NAG | B | 366 | 26.966 | 87.413 | 3.282 | 1.00 | 212.59 |
| 4576 | O3 | NAG | B | 366 | 26.061 | 86.895 | 2.319 | 1.00 | 212.59 |
| 4577 | C4 | NAG | B | 366 | 26.186 | 88.236 | 4.315 | 1.00 | 212.59 |
| 4578 | O4 | NAG | B | 366 | 25.698 | 89.443 | 3.682 | 1.00 | 212.59 |
| 4579 | C5 | NAG | B | 366 | 27.096 | 88.607 | 5.499 | 1.00 | 212.59 |
| 4580 | O5 | NAG | B | 366 | 27.723 | 87.423 | 6.036 | 1.00 | 212.59 |
| 4581 | C6 | NAG | B | 366 | 26.361 | 89.288 | 6.648 | 1.00 | 212.59 |
| 4582 | O6 | NAG | B | 366 | 27.276 | 89.790 | 7.613 | 1.00 | 212.59 |
| 4583 | C1 | NAG | B | 367 | 24.341 | 89.710 | 3.786 | 1.00 | 243.26 |
| 4584 | C2 | NAG | B | 367 | 24.090 | 91.194 | 3.541 | 1.00 | 243.26 |
| 4585 | N2 | NAG | B | 367 | 24.852 | 92.006 | 4.472 | 1.00 | 243.26 |
| 4586 | C7 | NAG | B | 367 | 25.846 | 92.768 | 4.025 | 1.00 | 243.26 |
| 4587 | O7 | NAG | B | 367 | 26.167 | 92.827 | 2.834 | 1.00 | 243.26 |
| 4588 | C8 | NAG | B | 367 | 26.602 | 93.591 | 5.058 | 1.00 | 243.26 |
| 4589 | C3 | NAG | B | 367 | 22.591 | 91.455 | 3.687 | 1.00 | 243.26 |
| 4590 | O3 | NAG | B | 367 | 22.313 | 92.825 | 3.445 | 1.00 | 243.26 |
| 4591 | C4 | NAG | B | 367 | 21.820 | 90.586 | 2.689 | 1.00 | 243.26 |
| 4592 | O4 | NAG | B | 367 | 20.423 | 90.749 | 2.897 | 1.00 | 243.26 |
| 4593 | C5 | NAG | B | 367 | 22.208 | 89.105 | 2.859 | 1.00 | 243.26 |
| 4594 | O5 | NAG | B | 367 | 23.647 | 88.941 | 2.791 | 1.00 | 243.26 |
| 4595 | C6 | NAG | B | 367 | 21.611 | 88.219 | 1.777 | 1.00 | 243.26 |
| 4596 | O6 | NAG | B | 367 | 22.614 | 87.692 | 0.915 | 1.00 | 243.26 |
| 4597 | CB | LYS | D | 4 | 55.929 | 67.814 | 61.471 | 1.00 | 249.69 |
| 4598 | CG | LYS | D | 4 | 55.569 | 66.389 | 61.069 | 1.00 | 249.69 |
| 4599 | CD | LYS | D | 4 | 55.219 | 65.523 | 62.280 | 1.00 | 249.69 |
| 4600 | CE | LYS | D | 4 | 54.831 | 64.103 | 61.856 | 1.00 | 249.69 |
| 4601 | NZ | LYS | D | 4 | 54.503 | 63.215 | 63.020 | 1.00 | 249.69 |
| 4602 | C | LYS | D | 4 | 54.982 | 68.782 | 59.376 | 1.00 | 226.67 |
| 4603 | O | LYS | D | 4 | 53.862 | 68.517 | 59.816 | 1.00 | 226.67 |
| 4604 | N | LYS | D | 4 | 56.551 | 70.102 | 60.766 | 1.00 | 226.67 |
| 4605 | CA | LYS | D | 4 | 56.206 | 68.740 | 60.282 | 1.00 | 226.67 |
| 4606 | N | PRO | D | 5 | 55.175 | 69.129 | 58.098 | 1.00 | 199.21 |
| 4607 | CD | PRO | D | 5 | 56.399 | 69.692 | 57.504 | 1.00 | 157.97 |
| 4608 | CA | PRO | D | 5 | 54.056 | 69.192 | 57.153 | 1.00 | 199.21 |
| 4609 | CB | PRO | D | 5 | 54.551 | 70.184 | 56.106 | 1.00 | 157.97 |
| 4610 | CG | PRO | D | 5 | 56.009 | 69.877 | 56.038 | 1.00 | 157.97 |
| 4611 | C | PRO | D | 5 | 53.742 | 67.819 | 56.558 | 1.00 | 199.21 |
| 4612 | O | PRO | D | 5 | 54.592 | 66.931 | 56.558 | 1.00 | 199.21 |
| 4613 | N | LYS | D | 6 | 52.523 | 67.641 | 56.064 | 1.00 | 205.80 |
| 4614 | CA | LYS | D | 6 | 52.136 | 66.371 | 55.468 | 1.00 | 205.80 |
| 4615 | CB | LYS | D | 6 | 51.395 | 65.500 | 56.489 | 1.00 | 249.69 |
| 4616 | CG | LYS | D | 6 | 51.007 | 64.131 | 55.942 | 1.00 | 249.69 |
| 4617 | CD | LYS | D | 6 | 50.433 | 63.220 | 57.018 | 1.00 | 249.69 |
| 4618 | CE | LYS | D | 6 | 50.116 | 61.838 | 56.448 | 1.00 | 249.69 |
| 4619 | NZ | LYS | D | 6 | 49.665 | 60.880 | 57.497 | 1.00 | 249.69 |
| 4620 | C | LYS | D | 6 | 51.263 | 66.599 | 54.246 | 1.00 | 205.80 |
| 4621 | O | LYS | D | 6 | 50.132 | 67.075 | 54.362 | 1.00 | 205.80 |
| 4622 | N | VAL | D | 7 | 51.797 | 66.245 | 53.080 | 1.00 | 180.35 |
| 4623 | CA | VAL | D | 7 | 51.082 | 66.425 | 51.823 | 1.00 | 180.35 |
| 4624 | CB | VAL | D | 7 | 52.002 | 66.148 | 50.636 | 1.00 | 112.97 |
| 4625 | CG1 | VAL | D | 7 | 51.369 | 66.692 | 49.350 | 1.00 | 112.97 |
| 4626 | CG2 | VAL | D | 7 | 53.374 | 66.752 | 50.884 | 1.00 | 112.97 |
| 4627 | C | VAL | D | 7 | 49.846 | 65.540 | 51.677 | 1.00 | 180.35 |
| 4628 | O | VAL | D | 7 | 49.935 | 64.315 | 51.772 | 1.00 | 180.35 |
| 4629 | N | SER | D | 8 | 48.699 | 66.171 | 51.443 | 1.00 | 191.62 |
| 4630 | CA | SER | D | 8 | 47.441 | 65.455 | 51.269 | 1.00 | 191.62 |
| 4631 | CB | SER | D | 8 | 46.339 | 66.118 | 52.114 | 1.00 | 215.34 |
| 4632 | OG | SER | D | 8 | 46.315 | 67.528 | 51.940 | 1.00 | 215.34 |
| 4633 | C | SER | D | 8 | 47.066 | 65.475 | 49.790 | 1.00 | 191.62 |
| 4634 | O | SER | D | 8 | 47.587 | 66.285 | 49.026 | 1.00 | 191.62 |
| 4635 | N | LEU | D | 9 | 46.175 | 64.579 | 49.374 | 1.00 | 183.49 |
| 4636 | CA | LEU | D | 9 | 45.753 | 64.552 | 47.973 | 1.00 | 183.49 |
| 4637 | CB | LEU | D | 9 | 46.289 | 63.316 | 47.250 | 1.00 | 153.82 |
| 4638 | CG | LEU | D | 9 | 47.793 | 63.054 | 47.150 | 1.00 | 153.82 |
| 4639 | CD1 | LEU | D | 9 | 48.011 | 61.992 | 46.080 | 1.00 | 153.82 |
| 4640 | CD2 | LEU | D | 9 | 48.557 | 64.312 | 46.800 | 1.00 | 153.82 |
| 4641 | C | LEU | D | 9 | 44.243 | 64.561 | 47.836 | 1.00 | 183.49 |
| 4642 | O | LEU | D | 9 | 43.522 | 64.243 | 48.781 | 1.00 | 183.49 |
| 4643 | N | ASN | D | 10 | 43.769 | 64.929 | 46.650 | 1.00 | 161.08 |
| 4644 | CA | ASN | D | 10 | 42.340 | 64.954 | 46.383 | 1.00 | 161.08 |
| 4645 | CB | ASN | D | 10 | 41.701 | 66.192 | 46.999 | 1.00 | 220.60 |
| 4646 | CG | ASN | D | 10 | 40.195 | 66.089 | 47.052 | 1.00 | 220.60 |
| 4647 | OD1 | ASN | D | 10 | 39.645 | 65.222 | 47.732 | 1.00 | 220.60 |
| 4648 | ND2 | ASN | D | 10 | 39.515 | 66.966 | 46.328 | 1.00 | 220.60 |
| 4649 | C | ASN | D | 10 | 42.077 | 64.931 | 44.883 | 1.00 | 161.08 |
| 4650 | O | ASN | D | 10 | 42.376 | 65.903 | 44.187 | 1.00 | 161.08 |
| 4651 | N | PRO | D | 11 | 41.505 | 63.830 | 44.368 | 1.00 | 193.66 |
| 4652 | CD | PRO | D | 11 | 41.212 | 63.723 | 42.927 | 1.00 | 148.22 |
| 4653 | CA | PRO | D | 11 | 41.077 | 62.602 | 45.052 | 1.00 | 193.66 |
| 4654 | CB | PRO | D | 11 | 40.656 | 61.698 | 43.905 | 1.00 | 148.22 |
| 4655 | CG | PRO | D | 11 | 40.146 | 62.658 | 42.901 | 1.00 | 148.22 |
| 4656 | C | PRO | D | 11 | 42.161 | 61.959 | 45.924 | 1.00 | 193.66 |
| 4657 | O | PRO | D | 11 | 43.336 | 62.325 | 45.849 | 1.00 | 193.66 |
| 4658 | N | PRO | D | 12 | 41.772 | 60.982 | 46.769 | 1.00 | 193.56 |
| 4659 | CD | PRO | D | 12 | 40.402 | 60.544 | 47.062 | 1.00 | 138.53 |
| 4660 | CA | PRO | D | 12 | 42.731 | 60.293 | 47.644 | 1.00 | 193.56 |
| 4661 | CB | PRO | D | 12 | 41.824 | 59.503 | 48.588 | 1.00 | 138.53 |
| 4662 | CG | PRO | D | 12 | 40.494 | 60.225 | 48.520 | 1.00 | 138.53 |
| 4663 | C | PRO | D | 12 | 43.633 | 59.379 | 46.825 | 1.00 | 193.56 |
| 4664 | O | PRO | D | 12 | 44.775 | 59.096 | 47.204 | 1.00 | 193.56 |
| 4665 | N | TRP | D | 13 | 43.081 | 53.919 | 45.700 | 1.00 | 115.99 |
| 4666 | CA | TRP | D | 13 | 43.745 | 58.039 | 44.727 | 1.00 | 115.99 |
| 4667 | CB | TRP | D | 13 | 42.854 | 57.917 | 43.495 | 1.00 | 155.11 |
| 4668 | CG | TRP | D | 13 | 41.432 | 57.624 | 43.839 | 1.00 | 155.11 |
| 4669 | CD2 | TRP | D | 13 | 40.964 | 56.869 | 44.959 | 1.00 | 155.11 |
| 4670 | CE2 | TRP | D | 13 | 39.560 | 56.822 | 44.873 | 1.00 | 155.11 |
| 4671 | CE3 | TRP | D | 13 | 41.596 | 56.233 | 46.022 | 1.00 | 155.11 |
| 4672 | CD1 | TRP | D | 13 | 40.330 | 57.988 | 43.138 | 1.00 | 155.11 |
| 4673 | NE1 | TRP | D | 13 | 39.192 | 57.509 | 43.751 | 1.00 | 155.11 |
| 4674 | CZ2 | TRP | D | 13 | 38.778 | 56.160 | 45.808 | 1.00 | 155.11 |
| 4675 | CZ3 | TRP | D | 13 | 40.824 | 55.569 | 46.957 | 1.00 | 155.11 |
| 4676 | CH2 | TRP | D | 13 | 39.426 | 55.538 | 46.844 | 1.00 | 155.11 |
| 4677 | C | TRP | D | 13 | 45.119 | 58.540 | 44.288 | 1.00 | 115.99 |
| 4678 | O | TRP | D | 13 | 45.213 | 59.565 | 43.618 | 1.00 | 115.99 |
| 4679 | N | ASN | D | 14 | 46.176 | 57.810 | 44.644 | 1.00 | 127.73 |
| 4680 | CA | ASN | D | 14 | 47.541 | 58.211 | 44.268 | 1.00 | 127.73 |
| 4681 | CB | ASN | D | 14 | 48.472 | 58.128 | 45.485 | 1.00 | 164.43 |
| 4682 | CG | ASN | D | 14 | 48.644 | 56.717 | 45.996 | 1.00 | 164.43 |
| 4683 | OD1 | ASN | D | 14 | 47.674 | 56.039 | 46.369 | 1.00 | 164.43 |
| 4684 | ND2 | ASN | D | 14 | 49.888 | 56.265 | 46.023 | 1.00 | 164.43 |
| 4685 | C | ASN | D | 14 | 48.124 | 57.395 | 43.094 | 1.00 | 127.73 |
| 4686 | O | ASN | D | 14 | 49.361 | 57.291 | 42.929 | 1.00 | 127.73 |
| 4687 | N | ARG | D | 15 | 47.202 | 56.825 | 42.304 | 1.00 | 124.12 |
| 4688 | CA | ARG | D | 15 | 47.484 | 56.018 | 41.111 | 1.00 | 124.12 |
| 4689 | CB | ARG | D | 15 | 47.249 | 54.517 | 41.374 | 1.00 | 138.52 |
| 4690 | CG | ARG | D | 15 | 47.935 | 53.919 | 42.607 | 1.00 | 138.52 |
| 4691 | CD | ARG | D | 15 | 47.775 | 52.394 | 42.630 | 1.00 | 138.52 |
| 4692 | NE | ARG | D | 15 | 48.696 | 51.717 | 41.716 | 1.00 | 138.52 |
| 4693 | CZ | ARG | D | 15 | 48.387 | 50.631 | 41.012 | 1.00 | 138.52 |
| 4694 | NH1 | ARG | D | 15 | 47.175 | 50.091 | 41.111 | 1.00 | 138.52 |
| 4695 | NH2 | ARG | D | 15 | 49.292 | 50.084 | 40.211 | 1.00 | 138.52 |
| 4696 | C | ARG | D | 15 | 46.436 | 56.487 | 40.117 | 1.00 | 124.12 |
| 4697 | O | ARG | D | 15 | 45.277 | 56.068 | 40.197 | 1.00 | 124.12 |
| 4698 | N | ILE | D | 16 | 46.825 | 57.344 | 39.182 | 1.00 | 134.05 |
| 4699 | CA | ILE | D | 16 | 45.853 | 57.861 | 38.222 | 1.00 | 134.05 |
| 4700 | CB | ILE | D | 16 | 45.666 | 59.359 | 38.405 | 1.00 | 185.30 |
| 4701 | CG2 | ILE | D | 16 | 44.824 | 59.635 | 39.645 | 1.00 | 185.30 |
| 4702 | CG1 | ILE | D | 16 | 47.047 | 60.016 | 38.464 | 1.00 | 185.30 |
| 4703 | CD1 | ILE | D | 16 | 47.030 | 61.514 | 38.379 | 1.00 | 185.30 |
| 4704 | C | ILE | D | 16 | 46.150 | 57.638 | 36.740 | 1.00 | 134.05 |
| 4705 | O | ILE | D | 16 | 47.301 | 57.474 | 36.330 | 1.00 | 134.05 |
| 4706 | N | PHE | D | 17 | 45.088 | 57.650 | 35.944 | 1.00 | 221.22 |
| 4707 | CA | PHE | D | 17 | 45.198 | 57.475 | 34.508 | 1.00 | 221.22 |
| 4708 | CB | PHE | D | 17 | 43.814 | 57.258 | 33.908 | 1.00 | 170.58 |
| 4709 | CG | PHE | D | 17 | 43.398 | 55.818 | 33.833 | 1.00 | 170.58 |
| 4710 | CD1 | PHE | D | 17 | 42.060 | 55.454 | 34.005 | 1.00 | 170.58 |
| 4711 | CD2 | PHE | D | 17 | 44.330 | 54.832 | 33.544 | 1.00 | 170.58 |
| 4712 | CE1 | PHE | D | 17 | 41.658 | 54.141 | 33.886 | 1.00 | 170.58 |
| 4713 | CE2 | PHE | D | 17 | 43.932 | 53.511 | 33.422 | 1.00 | 170.58 |
| 4714 | CZ | PHE | D | 17 | 42.590 | 53.167 | 33.594 | 1.00 | 170.58 |
| 4715 | C | PHE | D | 17 | 45.825 | 58.706 | 33.880 | 1.00 | 221.22 |
| 4716 | O | PHE | D | 17 | 46.106 | 59.689 | 34.562 | 1.00 | 221.22 |
| 4717 | N | LYS | D | 18 | 46.023 | 58.646 | 32.569 | 1.00 | 189.75 |
| 4718 | CA | LYS | D | 18 | 46.615 | 59.743 | 31.808 | 1.00 | 189.75 |
| 4719 | CB | LYS | D | 18 | 47.255 | 59.178 | 30.538 | 1.00 | 249.69 |
| 4720 | CG | LYS | D | 18 | 47.978 | 60.189 | 29.663 | 1.00 | 249.69 |
| 4721 | CD | LYS | D | 18 | 48.719 | 59.471 | 28.531 | 1.00 | 249.69 |
| 4722 | CE | LYS | D | 18 | 49.392 | 60.449 | 27.572 | 1.00 | 249.69 |
| 4723 | NZ | LYS | D | 18 | 48.405 | 61.242 | 26.779 | 1.00 | 249.69 |
| 4724 | C | LYS | D | 18 | 45.573 | 60.806 | 31.450 | 1.00 | 189.75 |
| 4725 | O | LYS | D | 18 | 44.509 | 60.493 | 30.912 | 1.00 | 189.75 |
| 4726 | N | GLY | D | 19 | 45.887 | 62.060 | 31.766 | 1.00 | 246.53 |
| 4727 | CA | GLY | D | 19 | 44.979 | 63.151 | 31.467 | 1.00 | 246.53 |
| 4728 | C | GLY | D | 19 | 44.072 | 63.582 | 32.607 | 1.00 | 246.53 |
| 4729 | O | GLY | D | 19 | 43.415 | 64.620 | 32.512 | 1.00 | 246.53 |
| 4730 | N | GLU | D | 20 | 44.029 | 62.794 | 33.680 | 1.00 | 150.48 |
| 4731 | CA | GLU | D | 20 | 43.189 | 63.103 | 34.849 | 1.00 | 150.48 |
| 4732 | CB | GLU | D | 20 | 42.969 | 61.840 | 35.704 | 1.00 | 195.02 |
| 4733 | CG | GLU | D | 20 | 42.534 | 60.576 | 34.943 | 1.00 | 195.02 |
| 4734 | CD | GLU | D | 20 | 42.202 | 59.403 | 35.877 | 1.00 | 195.02 |
| 4735 | OE1 | GLU | D | 20 | 43.045 | 59.054 | 36.735 | 1.00 | 195.02 |
| 4736 | OE2 | GLU | D | 20 | 41.093 | 58.831 | 35.748 | 1.00 | 195.02 |
| 4737 | C | GLU | D | 20 | 43.844 | 64.181 | 35.717 | 1.00 | 150.48 |
| 4738 | O | GLU | D | 20 | 45.062 | 64.375 | 35.641 | 1.00 | 150.48 |
| 4739 | N | ASN | D | 21 | 43.054 | 64.870 | 36.545 | 1.00 | 166.05 |
| 4740 | CA | ASN | D | 21 | 43.621 | 65.916 | 37.407 | 1.00 | 166.05 |
| 4741 | CB | ASN | D | 21 | 42.869 | 67.242 | 37.240 | 1.00 | 249.69 |
| 4742 | CG | ASN | D | 21 | 42.390 | 67.487 | 35.822 | 1.00 | 249.69 |
| 4743 | OD1 | ASN | D | 21 | 43.129 | 67.337 | 34.850 | 1.00 | 249.69 |
| 4744 | ND2 | ASN | D | 21 | 41.130 | 67.891 | 35.723 | 1.00 | 249.69 |
| 4745 | C | ASN | D | 21 | 43.632 | 65.566 | 38.903 | 1.00 | 166.05 |
| 4746 | O | ASN | D | 21 | 42.697 | 64.941 | 39.418 | 1.00 | 166.05 |
| 4747 | N | VAL | D | 22 | 44.685 | 66.001 | 39.593 | 1.00 | 232.99 |
| 4748 | CA | VAL | D | 22 | 44.836 | 65.753 | 41.022 | 1.00 | 232.99 |
| 4749 | CB | VAL | D | 22 | 45.811 | 64.598 | 41.274 | 1.00 | 144.01 |
| 4750 | CG1 | VAL | D | 22 | 47.232 | 65.032 | 40.944 | 1.00 | 144.01 |
| 4751 | CG2 | VAL | D | 22 | 45.711 | 64.149 | 42.718 | 1.00 | 144.01 |
| 4752 | C | VAL | D | 22 | 45.367 | 67.003 | 41.726 | 1.00 | 232.99 |
| 4753 | O | VAL | D | 22 | 46.132 | 67.762 | 41.135 | 1.00 | 232.99 |
| 4754 | N | THR | D | 23 | 44.977 | 67.204 | 42.986 | 1.00 | 149.70 |
| 4755 | CA | THR | D | 23 | 45.409 | 68.376 | 43.760 | 1.00 | 149.70 |
| 4756 | CB | THR | D | 23 | 44.185 | 69.205 | 44.222 | 1.00 | 239.97 |
| 4757 | OG1 | THR | D | 23 | 43.328 | 69.469 | 43.101 | 1.00 | 239.97 |
| 4758 | CG2 | THR | D | 23 | 44.641 | 70.527 | 44.839 | 1.00 | 239.97 |
| 4759 | C | THR | D | 23 | 46.242 | 68.035 | 45.013 | 1.00 | 149.70 |
| 4760 | O | THR | D | 23 | 45.802 | 67.260 | 45.865 | 1.00 | 149.70 |
| 4761 | N | LEU | D | 24 | 47.426 | 68.633 | 45.130 | 1.00 | 183.97 |
| 4762 | CA | LEU | D | 24 | 48.291 | 68.390 | 46.282 | 1.00 | 183.97 |
| 4763 | CB | LEU | D | 24 | 49.736 | 68.117 | 45.847 | 1.00 | 149.27 |
| 4764 | CG | LEU | D | 24 | 50.067 | 67.414 | 44.528 | 1.00 | 149.27 |
| 4765 | CD1 | LEU | D | 24 | 51.537 | 66.998 | 44.556 | 1.00 | 149.27 |
| 4766 | CD2 | LEU | D | 24 | 49.182 | 66.203 | 44.315 | 1.00 | 149.27 |
| 4767 | C | LEU | D | 24 | 48.292 | 69.583 | 47.247 | 1.00 | 183.97 |
| 4768 | O | LEU | D | 24 | 48.884 | 70.627 | 46.961 | 1.00 | 183.97 |
| 4769 | N | THR | D | 25 | 47.642 | 69.414 | 48.394 | 1.00 | 179.50 |
| 4770 | CA | THR | D | 25 | 47.555 | 70.453 | 49.422 | 1.00 | 179.50 |
| 4771 | CB | THR | D | 25 | 46.149 | 70.455 | 50.074 | 1.00 | 206.28 |
| 4772 | OG1 | THR | D | 25 | 45.152 | 70.641 | 49.059 | 1.00 | 206.28 |
| 4773 | CG2 | THR | D | 25 | 46.035 | 71.567 | 51.109 | 1.00 | 206.28 |
| 4774 | C | THR | D | 25 | 48.608 | 70.207 | 50.510 | 1.00 | 179.50 |
| 4775 | O | THR | D | 25 | 48.762 | 69.083 | 50.983 | 1.00 | 179.50 |
| 4776 | N | CYS | D | 26 | 49.321 | 71.258 | 50.907 | 1.00 | 232.65 |
| 4777 | CA | CYS | D | 26 | 50.349 | 71.137 | 51.941 | 1.00 | 232.65 |
| 4778 | C | CYS | D | 26 | 49.723 | 71.185 | 53.337 | 1.00 | 232.65 |
| 4779 | O | CYS | D | 26 | 48.767 | 71.928 | 53.562 | 1.00 | 232.65 |
| 4780 | CB | CYS | D | 26 | 51.377 | 72.252 | 51.784 | 1.00 | 181.06 |
| 4781 | SG | CYS | D | 26 | 52.866 | 72.078 | 52.815 | 1.00 | 181.06 |
| 4782 | N | ASN | D | 27 | 50.274 | 70.400 | 54.266 | 1.00 | 211.42 |
| 4783 | CA | ASN | D | 27 | 49.762 | 70.299 | 55.633 | 1.00 | 211.42 |
| 4784 | CB | ASN | D | 27 | 50.909 | 70.245 | 56.640 | 1.00 | 249.69 |
| 4785 | CG | ASN | D | 27 | 50.435 | 69.883 | 58.041 | 1.00 | 249.69 |
| 4786 | OD1 | ASN | D | 27 | 49.691 | 68.915 | 58.228 | 1.00 | 249.69 |
| 4787 | ND2 | ASN | D | 27 | 50.866 | 70.658 | 59.035 | 1.00 | 249.69 |
| 4788 | C | ASN | D | 27 | 48.782 | 71.397 | 56.027 | 1.00 | 211.42 |
| 4789 | O | ASN | D | 27 | 49.176 | 72.473 | 56.478 | 1.00 | 211.42 |
| 4790 | N | GLY | D | 28 | 47.499 | 71.096 | 55.848 | 1.00 | 232.09 |
| 4791 | CA | GLY | D | 28 | 46.428 | 72.024 | 56.166 | 1.00 | 232.09 |
| 4792 | C | GLY | D | 28 | 45.145 | 71.413 | 55.640 | 1.00 | 232.09 |
| 4793 | O | GLY | D | 28 | 45.028 | 71.150 | 54.442 | 1.00 | 232.09 |
| 4794 | N | ASN | D | 29 | 44.183 | 71.179 | 56.527 | 1.00 | 249.69 |
| 4795 | CA | ASN | D | 29 | 42.916 | 70.564 | 56.138 | 1.00 | 249.69 |
| 4796 | CB | ASN | D | 29 | 42.185 | 70.041 | 57.392 | 1.00 | 249.66 |
| 4797 | CG | ASN | D | 29 | 40.997 | 69.139 | 57.060 | 1.00 | 249.66 |
| 4798 | OD1 | ASN | D | 29 | 40.786 | 68.758 | 55.903 | 1.00 | 249.66 |
| 4799 | ND2 | ASN | D | 29 | 40.223 | 68.786 | 58.082 | 1.00 | 249.66 |
| 4800 | C | ASN | D | 29 | 41.992 | 71.490 | 55.331 | 1.00 | 249.69 |
| 4801 | O | ASN | D | 29 | 41.536 | 71.122 | 54.239 | 1.00 | 249.69 |
| 4802 | N | ASN | D | 30 | 41.730 | 72.692 | 55.843 | 1.00 | 249.69 |
| 4803 | CA | ASN | D | 30 | 40.830 | 73.606 | 55.146 | 1.00 | 249.69 |
| 4804 | CB | ASN | D | 30 | 39.518 | 73.728 | 55.941 | 1.00 | 249.69 |
| 4805 | CG | ASN | D | 30 | 38.761 | 72.406 | 56.039 | 1.00 | 249.69 |
| 4806 | OD1 | ASN | D | 30 | 38.314 | 72.011 | 57.123 | 1.00 | 249.69 |
| 4807 | ND2 | ASN | D | 30 | 38.607 | 71.718 | 54.903 | 1.00 | 249.69 |
| 4808 | C | ASN | D | 30 | 41.378 | 75.001 | 54.827 | 1.00 | 249.69 |
| 4809 | O | ASN | D | 30 | 41.596 | 75.328 | 53.656 | 1.00 | 249.69 |
| 4810 | N | PHE | D | 31 | 41.599 | 75.820 | 55.856 | 1.00 | 244.83 |
| 4811 | CA | PHE | D | 31 | 42.085 | 77.179 | 55.639 | 1.00 | 244.83 |
| 4812 | CB | PHE | D | 31 | 41.091 | 78.191 | 56.235 | 1.00 | 249.48 |
| 4813 | CG | PHE | D | 31 | 39.675 | 78.021 | 55.735 | 1.00 | 249.48 |
| 4814 | CD1 | PHE | D | 31 | 38.843 | 77.036 | 56.268 | 1.00 | 249.48 |
| 4815 | CD2 | PHE | D | 31 | 39.182 | 78.824 | 54.704 | 1.00 | 249.48 |
| 4816 | CE1 | PHE | D | 31 | 37.543 | 76.849 | 55.782 | 1.00 | 249.48 |
| 4817 | CE2 | PHE | D | 31 | 37.880 | 78.644 | 54.208 | 1.00 | 249.48 |
| 4818 | CZ | PHE | D | 31 | 37.061 | 77.654 | 54.750 | 1.00 | 249.48 |
| 4819 | C | PHE | D | 31 | 43.496 | 77.454 | 56.169 | 1.00 | 244.83 |
| 4820 | O | PHE | D | 31 | 43.773 | 77.287 | 57.358 | 1.00 | 244.83 |
| 4821 | N | PHE | D | 32 | 44.378 | 77.888 | 55.265 | 1.00 | 249.69 |
| 4822 | CA | PHE | D | 32 | 45.775 | 78.197 | 55.591 | 1.00 | 249.69 |
| 4823 | CB | PHE | D | 32 | 46.711 | 77.304 | 54.761 | 1.00 | 249.69 |
| 4824 | CG | PHE | D | 32 | 48.130 | 77.274 | 55.266 | 1.00 | 249.69 |
| 4825 | CD1 | PHE | D | 32 | 48.435 | 76.713 | 56.514 | 1.00 | 249.69 |
| 4826 | CD2 | PHE | D | 32 | 49.164 | 77.800 | 54.494 | 1.00 | 249.69 |
| 4827 | CE1 | PHE | D | 32 | 49.752 | 76.678 | 56.983 | 1.00 | 249.69 |
| 4828 | CE2 | PHE | D | 32 | 50.487 | 77.770 | 54.954 | 1.00 | 249.69 |
| 4829 | CZ | PHE | D | 32 | 50.779 | 77.207 | 56.198 | 1.00 | 249.69 |
| 4830 | C | PHE | D | 32 | 46.086 | 79.681 | 55.325 | 1.00 | 249.69 |
| 4831 | O | PHE | D | 32 | 45.300 | 80.381 | 54.671 | 1.00 | 249.69 |
| 4832 | N | GLU | D | 33 | 47.241 | 80.150 | 55.802 | 1.00 | 237.86 |
| 4833 | CA | GLU | D | 33 | 47.603 | 81.555 | 55.640 | 1.00 | 237.86 |
| 4834 | CB | GLU | D | 33 | 47.766 | 82.184 | 57.029 | 1.00 | 249.69 |
| 4835 | CG | GLU | D | 33 | 47.688 | 83.708 | 57.034 | 1.00 | 249.69 |
| 4836 | CD | GLU | D | 33 | 46.478 | 84.238 | 56.256 | 1.00 | 249.69 |
| 4837 | OE1 | GLU | D | 33 | 45.351 | 83.734 | 56.476 | 1.00 | 249.69 |
| 4838 | OE2 | GLU | D | 33 | 46.656 | 85.163 | 55.429 | 1.00 | 249.69 |
| 4839 | C | GLU | D | 33 | 48.821 | 81.908 | 54.779 | 1.00 | 237.86 |
| 4840 | O | GLU | D | 33 | 48.729 | 82.759 | 53.896 | 1.00 | 237.86 |
| 4841 | N | VAL | D | 34 | 49.957 | 81.269 | 55.041 | 1.00 | 249.69 |
| 4842 | CA | VAL | D | 34 | 51.188 | 81.553 | 54.302 | 1.00 | 249.69 |
| 4843 | CB | VAL | D | 34 | 52.357 | 80.686 | 54.850 | 1.00 | 245.39 |
| 4844 | CG1 | VAL | D | 34 | 53.648 | 81.015 | 54.126 | 1.00 | 245.39 |
| 4845 | CG2 | VAL | D | 34 | 52.522 | 80.928 | 56.343 | 1.00 | 245.39 |
| 4846 | C | VAL | D | 34 | 51.103 | 81.386 | 52.773 | 1.00 | 249.69 |
| 4847 | O | VAL | D | 34 | 50.330 | 80.567 | 52.254 | 1.00 | 249.69 |
| 4848 | N | SER | D | 35 | 51.900 | 82.188 | 52.065 | 1.00 | 249.69 |
| 4849 | CA | SER | D | 35 | 51.963 | 82.160 | 50.603 | 1.00 | 249.69 |
| 4850 | CB | SER | D | 35 | 51.850 | 83.577 | 50.033 | 1.00 | 241.64 |
| 4851 | OG | SER | D | 35 | 53.028 | 84.324 | 50.300 | 1.00 | 241.64 |
| 4852 | C | SER | D | 35 | 53.307 | 81.559 | 50.188 | 1.00 | 249.69 |
| 4853 | O | SER | D | 35 | 53.587 | 81.400 | 48.997 | 1.00 | 249.69 |
| 4854 | N | SER | D | 36 | 54.137 | 81.243 | 51.184 | 1.00 | 249.69 |
| 4855 | CA | SER | D | 36 | 55.455 | 80.654 | 50.953 | 1.00 | 249.69 |
| 4856 | CB | SER | D | 36 | 56.516 | 81.325 | 51.841 | 1.00 | 249.69 |
| 4857 | OG | SER | D | 36 | 56.379 | 80.950 | 53.201 | 1.00 | 249.69 |
| 4858 | C | SER | D | 36 | 55.430 | 79.150 | 51.227 | 1.00 | 249.69 |
| 4859 | O | SER | D | 36 | 55.650 | 78.692 | 52.354 | 1.00 | 249.69 |
| 4860 | N | THR | D | 37 | 55.147 | 78.389 | 50.176 | 1.00 | 216.36 |
| 4861 | CA | THR | D | 37 | 55.091 | 76.944 | 50.267 | 1.00 | 216.36 |
| 4862 | CB | THR | D | 37 | 53.651 | 76.439 | 50.029 | 1.00 | 218.49 |
| 4863 | OG1 | THR | D | 37 | 52.773 | 77.009 | 51.009 | 1.00 | 218.49 |
| 4864 | CG2 | THR | D | 37 | 53.595 | 74.930 | 50.138 | 1.00 | 218.49 |
| 4865 | C | THR | D | 37 | 56.027 | 76.399 | 49.193 | 1.00 | 216.36 |
| 4866 | O | THR | D | 37 | 56.067 | 76.908 | 48.070 | 1.00 | 216.36 |
| 4867 | N | LYS | D | 38 | 56.792 | 75.372 | 49.539 | 1.00 | 249.68 |
| 4868 | CA | LYS | D | 38 | 57.737 | 74.790 | 48.591 | 1.00 | 249.68 |
| 4869 | CB | LYS | D | 38 | 59.115 | 74.638 | 49.265 | 1.00 | 248.74 |
| 4870 | CG | LYS | D | 38 | 59.701 | 75.962 | 49.786 | 1.00 | 248.74 |
| 4871 | CD | LYS | D | 38 | 61.060 | 75.782 | 50.467 | 1.00 | 248.74 |
| 4872 | CE | LYS | D | 38 | 61.627 | 77.123 | 50.933 | 1.00 | 248.74 |
| 4873 | NZ | LYS | D | 38 | 62.953 | 76.993 | 51.599 | 1.00 | 248.74 |
| 4874 | C | LYS | D | 38 | 57.259 | 73.445 | 48.044 | 1.00 | 249.68 |
| 4875 | O | LYS | D | 38 | 56.626 | 72.660 | 48.747 | 1.00 | 249.68 |
| 4876 | N | TRP | D | 39 | 57.552 | 73.197 | 46.775 | 1.00 | 200.98 |
| 4877 | CA | TRP | D | 39 | 57.178 | 71.944 | 46.137 | 1.00 | 200.98 |
| 4878 | CB | TRP | D | 39 | 56.085 | 72.188 | 45.096 | 1.00 | 193.49 |
| 4879 | CG | TRP | D | 39 | 54.754 | 72.634 | 45.638 | 1.00 | 193.49 |
| 4880 | CD2 | TRP | D | 39 | 53.910 | 71.924 | 46.562 | 1.00 | 193.49 |
| 4881 | CE2 | TRP | D | 39 | 52.708 | 72.649 | 46.669 | 1.00 | 193.49 |
| 4882 | CE3 | TRP | D | 39 | 54.053 | 70.747 | 47.307 | 1.00 | 193.49 |
| 4883 | CD1 | TRP | D | 39 | 54.051 | 73.727 | 45.247 | 1.00 | 193.49 |
| 4884 | NE1 | TRP | D | 39 | 52.822 | 73.745 | 45.854 | 1.00 | 193.49 |
| 4885 | CZ2 | TRP | D | 39 | 51.648 | 72.242 | 47.487 | 1.00 | 193.49 |
| 4886 | CZ3 | TRP | D | 39 | 52.994 | 70.335 | 48.124 | 1.00 | 193.49 |
| 4887 | CH2 | TRP | D | 39 | 51.807 | 71.084 | 48.204 | 1.00 | 193.49 |
| 4888 | C | TRP | D | 39 | 58.428 | 71.393 | 45.451 | 1.00 | 200.98 |
| 4889 | O | TRP | D | 39 | 59.127 | 72.127 | 44.763 | 1.00 | 200.98 |
| 4890 | N | PHE | D | 40 | 58.712 | 70.108 | 45.635 | 1.00 | 160.00 |
| 4891 | CA | PHE | D | 40 | 59.896 | 69.514 | 45.027 | 1.00 | 160.00 |
| 4892 | CB | PHE | D | 40 | 60.943 | 69.163 | 46.100 | 1.00 | 228.90 |
| 4893 | CG | PHE | D | 40 | 61.397 | 70.336 | 46.936 | 1.00 | 228.90 |
| 4894 | CD1 | PHE | D | 40 | 60.620 | 70.798 | 47.999 | 1.00 | 228.90 |
| 4895 | CD2 | PHE | D | 40 | 62.611 | 70.962 | 46.677 | 1.00 | 228.90 |
| 4896 | CE1 | PHE | D | 40 | 61.049 | 71.862 | 48.794 | 1.00 | 228.90 |
| 4897 | CE2 | PHE | D | 40 | 63.046 | 72.028 | 47.465 | 1.00 | 228.90 |
| 4898 | CZ | PHE | D | 40 | 62.263 | 72.477 | 48.526 | 1.00 | 228.90 |
| 4899 | C | PHE | D | 40 | 59.620 | 68.266 | 44.183 | 1.00 | 160.00 |
| 4900 | O | PHE | D | 40 | 59.908 | 67.142 | 44.615 | 1.00 | 160.00 |
| 4901 | N | HIS | D | 41 | 59.088 | 68.469 | 42.976 | 1.00 | 161.00 |
| 4902 | CA | HIS | D | 41 | 58.786 | 67.371 | 42.052 | 1.00 | 161.00 |
| 4903 | CB | HIS | D | 41 | 58.044 | 67.915 | 40.844 | 1.00 | 195.26 |
| 4904 | CG | HIS | D | 41 | 57.679 | 66.868 | 39.847 | 1.00 | 195.26 |
| 4905 | CD2 | HIS | D | 41 | 57.680 | 66.881 | 38.493 | 1.00 | 195.26 |
| 4906 | ND1 | HIS | D | 41 | 57.205 | 65.626 | 40.216 | 1.00 | 195.26 |
| 4907 | CE1 | HIS | D | 41 | 56.928 | 64.923 | 39.133 | 1.00 | 195.26 |
| 4908 | NE2 | HIS | D | 41 | 57.207 | 65.660 | 38.073 | 1.00 | 195.26 |
| 4909 | C | HIS | D | 41 | 60.056 | 66.641 | 41.588 | 1.00 | 161.00 |
| 4910 | O | HIS | D | 41 | 60.798 | 67.153 | 40.751 | 1.00 | 161.00 |
| 4911 | N | ASN | D | 42 | 60.280 | 65.437 | 42.118 | 1.00 | 193.10 |
| 4912 | CA | ASN | D | 42 | 61.464 | 64.633 | 41.801 | 1.00 | 193.10 |
| 4913 | CB | ASN | D | 42 | 61.638 | 64.471 | 40.281 | 1.00 | 195.59 |
| 4914 | CG | ASN | D | 42 | 60.700 | 63.415 | 39.686 | 1.00 | 195.59 |
| 4915 | OD1 | ASN | D | 42 | 59.501 | 63.435 | 39.953 | 1.00 | 195.59 |
| 4916 | ND2 | ASN | D | 42 | 61.240 | 62.506 | 38.873 | 1.00 | 195.59 |
| 4917 | C | ASN | D | 42 | 62.690 | 65.318 | 42.397 | 1.00 | 193.10 |
| 4918 | O | ASN | D | 42 | 63.810 | 65.146 | 41.919 | 1.00 | 193.10 |
| 4919 | N | GLY | D | 43 | 62.462 | 66.092 | 43.456 | 1.00 | 217.79 |
| 4920 | CA | GLY | D | 43 | 63.540 | 66.809 | 44.120 | 1.00 | 217.79 |
| 4921 | C | GLY | D | 43 | 63.681 | 68.227 | 43.596 | 1.00 | 217.79 |
| 4922 | O | GLY | D | 43 | 63.883 | 69.166 | 44.372 | 1.00 | 217.79 |
| 4923 | N | SER | D | 44 | 63.567 | 68.373 | 42.275 | 1.00 | 249.69 |
| 4924 | CA | SER | D | 44 | 63.675 | 69.669 | 41.598 | 1.00 | 249.69 |
| 4925 | CB | SER | D | 44 | 63.508 | 69.497 | 40.083 | 1.00 | 225.92 |
| 4926 | OG | SER | D | 44 | 64.485 | 68.631 | 39.546 | 1.00 | 225.92 |
| 4927 | C | SER | D | 44 | 62.620 | 70.652 | 42.085 | 1.00 | 249.69 |
| 4928 | O | SER | D | 44 | 61.423 | 70.374 | 41.997 | 1.00 | 249.69 |
| 4929 | N | LEU | D | 45 | 63.056 | 71.806 | 42.579 | 1.00 | 241.20 |
| 4930 | CA | LEU | D | 45 | 62.110 | 72.805 | 43.063 | 1.00 | 241.20 |
| 4931 | CB | LEU | D | 45 | 62.841 | 74.084 | 43.488 | 1.00 | 237.73 |
| 4932 | CG | LEU | D | 45 | 61.948 | 75.186 | 44.070 | 1.00 | 237.73 |
| 4933 | CD1 | LEU | D | 45 | 61.151 | 74.631 | 45.239 | 1.00 | 237.73 |
| 4934 | CD2 | LEU | D | 45 | 62.798 | 76.363 | 44.515 | 1.00 | 237.73 |
| 4935 | C | LEU | D | 45 | 61.074 | 73.125 | 41.980 | 1.00 | 241.20 |
| 4936 | O | LEU | D | 45 | 61.365 | 73.051 | 40.783 | 1.00 | 241.20 |
| 4937 | N | SER | D | 46 | 59.865 | 73.470 | 42.416 | 1.00 | 233.99 |
| 4938 | CA | SER | D | 46 | 58.772 | 73.787 | 41.503 | 1.00 | 233.99 |
| 4939 | CB | SER | D | 46 | 57.494 | 73.050 | 41.932 | 1.00 | 249.22 |
| 4940 | OG | SER | D | 46 | 56.444 | 73.250 | 40.995 | 1.00 | 249.22 |
| 4941 | C | SER | D | 46 | 58.506 | 75.285 | 41.441 | 1.00 | 233.99 |
| 4942 | O | SER | D | 46 | 59.042 | 76.064 | 42.232 | 1.00 | 233.99 |
| 4943 | N | GLU | D | 47 | 57.648 | 75.671 | 40.502 | 1.00 | 249.69 |
| 4944 | CA | GLU | D | 47 | 57.306 | 77.074 | 40.285 | 1.00 | 249.69 |
| 4945 | CB | GLU | D | 47 | 57.093 | 77.311 | 38.786 | 1.00 | 249.69 |
| 4946 | CG | GLU | D | 47 | 58.330 | 77.026 | 37.945 | 1.00 | 249.69 |
| 4947 | CD | GLU | D | 47 | 58.089 | 77.248 | 36.471 | 1.00 | 249.69 |
| 4948 | OE1 | GLU | D | 47 | 57.260 | 76.516 | 35.888 | 1.00 | 249.69 |
| 4949 | OE2 | GLU | D | 47 | 58.728 | 78.156 | 35.899 | 1.00 | 249.69 |
| 4950 | C | GLU | D | 47 | 56.102 | 77.618 | 41.063 | 1.00 | 249.69 |
| 4951 | O | GLU | D | 47 | 55.889 | 78.827 | 41.111 | 1.00 | 249.69 |
| 4952 | N | GLU | D | 48 | 55.306 | 76.740 | 41.661 | 1.00 | 194.51 |
| 4953 | CA | GLU | D | 48 | 54.159 | 77.204 | 42.424 | 1.00 | 194.51 |
| 4954 | CB | GLU | D | 48 | 53.081 | 76.117 | 42.492 | 1.00 | 249.69 |
| 4955 | CG | GLU | D | 48 | 51.885 | 76.488 | 43.366 | 1.00 | 249.69 |
| 4956 | CD | GLU | D | 48 | 51.102 | 77.675 | 42.836 | 1.00 | 249.69 |
| 4957 | OE1 | GLU | D | 48 | 50.401 | 77.515 | 41.814 | 1.00 | 249.69 |
| 4958 | OE2 | GLU | D | 48 | 51.189 | 78.768 | 43.437 | 1.00 | 249.69 |
| 4959 | C | GLU | D | 48 | 54.611 | 77.576 | 43.826 | 1.00 | 194.51 |
| 4960 | O | GLU | D | 48 | 55.645 | 77.100 | 44.311 | 1.00 | 194.51 |
| 4961 | N | THR | D | 49 | 53.834 | 78.438 | 44.472 | 1.00 | 208.13 |
| 4962 | CA | THR | D | 49 | 54.134 | 78.889 | 45.831 | 1.00 | 208.13 |
| 4963 | CB | THR | D | 49 | 54.570 | 80.363 | 45.826 | 1.00 | 249.69 |
| 4964 | OG1 | THR | D | 49 | 53.575 | 81.153 | 45.159 | 1.00 | 249.69 |
| 4965 | CG2 | THR | D | 49 | 55.902 | 80.516 | 45.103 | 1.00 | 249.69 |
| 4966 | C | THR | D | 49 | 52.905 | 78.729 | 46.737 | 1.00 | 208.13 |
| 4967 | O | THR | D | 49 | 53.022 | 78.579 | 47.958 | 1.00 | 208.13 |
| 4968 | N | ASN | D | 50 | 51.725 | 78.765 | 46.127 | 1.00 | 217.97 |
| 4969 | CA | ASN | D | 50 | 50.477 | 78.601 | 46.861 | 1.00 | 217.97 |
| 4970 | CB | ASN | D | 50 | 49.294 | 78.643 | 45.885 | 1.00 | 202.82 |
| 4971 | CG | ASN | D | 50 | 47.963 | 78.742 | 46.592 | 1.00 | 202.82 |
| 4972 | OD1 | ASN | D | 50 | 47.874 | 78.441 | 47.781 | 1.00 | 202.82 |
| 4973 | ND2 | ASN | D | 50 | 46.924 | 79.156 | 45.865 | 1.00 | 202.82 |
| 4974 | C | ASN | D | 50 | 50.539 | 77.236 | 47.545 | 1.00 | 217.97 |
| 4975 | O | ASN | D | 50 | 51.219 | 76.338 | 47.072 | 1.00 | 217.97 |
| 4976 | N | SER | D | 51 | 49.834 | 77.071 | 48.653 | 1.00 | 198.36 |
| 4977 | CA | SER | D | 51 | 49.854 | 75.790 | 49.352 | 1.00 | 198.36 |
| 4978 | CB | SER | D | 51 | 49.201 | 75.920 | 50.738 | 1.00 | 249.69 |
| 4979 | OG | SER | D | 51 | 47.794 | 76.051 | 50.640 | 1.00 | 249.69 |
| 4980 | C | SER | D | 51 | 49.166 | 74.663 | 48.566 | 1.00 | 198.36 |
| 4981 | O | SER | D | 51 | 49.350 | 73.484 | 48.876 | 1.00 | 198.36 |
| 4982 | N | SER | D | 52 | 48.375 | 75.019 | 47.555 | 1.00 | 249.63 |
| 4983 | CA | SER | D | 52 | 47.679 | 74.019 | 46.739 | 1.00 | 249.63 |
| 4984 | CB | SER | D | 52 | 46.187 | 74.334 | 46.625 | 1.00 | 163.31 |
| 4985 | OG | SER | D | 52 | 45.563 | 74.298 | 47.891 | 1.00 | 163.31 |
| 4986 | C | SER | D | 52 | 48.258 | 73.922 | 45.336 | 1.00 | 249.63 |
| 4987 | O | SER | D | 52 | 48.011 | 74.780 | 44.489 | 1.00 | 249.63 |
| 4988 | N | LEU | D | 53 | 49.024 | 72.867 | 45.096 | 1.00 | 224.52 |
| 4989 | CA | LEU | D | 53 | 49.637 | 72.642 | 43.799 | 1.00 | 224.52 |
| 4990 | CB | LEU | D | 53 | 51.016 | 72.017 | 43.989 | 1.00 | 138.37 |
| 4991 | CG | LEU | D | 53 | 51.627 | 71.271 | 42.806 | 1.00 | 138.37 |
| 4992 | CD1 | LEU | D | 53 | 51.483 | 72.075 | 41.504 | 1.00 | 138.37 |
| 4993 | CD2 | LEU | D | 53 | 53.089 | 70.983 | 43.128 | 1.00 | 138.37 |
| 4994 | C | LEU | D | 53 | 48.761 | 71.740 | 42.951 | 1.00 | 224.52 |
| 4995 | O | LEU | D | 53 | 48.703 | 70.536 | 43.177 | 1.00 | 224.52 |
| 4996 | N | ASN | D | 54 | 48.080 | 72.325 | 41.973 | 1.00 | 200.53 |
| 4997 | CA | ASN | D | 54 | 47.219 | 71.538 | 41.115 | 1.00 | 200.53 |
| 4998 | CB | ASN | D | 54 | 46.121 | 72.402 | 40.513 | 1.00 | 228.73 |
| 4999 | CG | ASN | D | 54 | 45.105 | 72.817 | 41.535 | 1.00 | 228.73 |
| 5000 | OD1 | ASN | D | 54 | 44.559 | 71.982 | 42.255 | 1.00 | 228.73 |
| 5001 | ND2 | ASN | D | 54 | 44.839 | 74.111 | 41.608 | 1.00 | 228.73 |
| 5002 | C | ASN | D | 54 | 47.977 | 70.834 | 40.003 | 1.00 | 200.53 |
| 5003 | O | ASN | D | 54 | 49.102 | 71.221 | 39.639 | 1.00 | 200.53 |
| 5004 | N | ILE | D | 55 | 47.341 | 69.785 | 39.482 | 1.00 | 249.24 |
| 5005 | CA | ILE | D | 55 | 47.874 | 68.962 | 38.403 | 1.00 | 249.24 |
| 5006 | CB | ILE | D | 55 | 48.369 | 67.589 | 38.934 | 1.00 | 185.84 |
| 5007 | CG2 | ILE | D | 55 | 48.373 | 66.564 | 37.819 | 1.00 | 185.84 |
| 5008 | CG1 | ILE | D | 55 | 49.764 | 67.751 | 39.564 | 1.00 | 185.84 |
| 5009 | CD1 | ILE | D | 55 | 50.346 | 66.474 | 40.129 | 1.00 | 185.84 |
| 5010 | C | ILE | D | 55 | 46.742 | 68.738 | 37.416 | 1.00 | 249.24 |
| 5011 | O | ILE | D | 55 | 45.735 | 68.117 | 37.750 | 1.00 | 249.24 |
| 5012 | N | VAL | D | 56 | 46.903 | 69.251 | 36.205 | 1.00 | 249.05 |
| 5013 | CA | VAL | D | 56 | 45.870 | 69.090 | 35.198 | 1.00 | 249.05 |
| 5014 | CB | VAL | D | 56 | 45.719 | 70.362 | 34.349 | 1.00 | 249.53 |
| 5015 | CG1 | VAL | D | 56 | 44.353 | 70.370 | 33.664 | 1.00 | 249.53 |
| 5016 | CG2 | VAL | D | 56 | 45.886 | 71.589 | 35.230 | 1.00 | 249.53 |
| 5017 | C | VAL | D | 56 | 46.211 | 67.906 | 34.301 | 1.00 | 249.05 |
| 5018 | O | VAL | D | 56 | 46.980 | 67.034 | 34.704 | 1.00 | 249.05 |
| 5019 | N | ASN | D | 57 | 45.641 | 67.879 | 33.094 | 1.00 | 232.44 |
| 5020 | CA | ASN | D | 57 | 45.859 | 66.786 | 32.143 | 1.00 | 232.44 |
| 5021 | CB | ASN | D | 57 | 45.815 | 67.311 | 30.708 | 1.00 | 224.76 |
| 5022 | CG | ASN | D | 57 | 44.410 | 67.743 | 30.292 | 1.00 | 224.76 |
| 5023 | OD1 | ASN | D | 57 | 43.446 | 66.989 | 30.437 | 1.00 | 224.76 |
| 5024 | ND2 | ASN | D | 57 | 44.292 | 68.958 | 29.772 | 1.00 | 224.76 |
| 5025 | C | ASN | D | 57 | 47.149 | 66.031 | 32.410 | 1.00 | 232.44 |
| 5026 | O | ASN | D | 57 | 48.227 | 66.398 | 31.940 | 1.00 | 232.44 |
| 5027 | N | ALA | D | 58 | 46.988 | 64.963 | 33.186 | 1.00 | 180.25 |
| 5028 | CA | ALA | D | 58 | 48.062 | 64.086 | 33.631 | 1.00 | 180.25 |
| 5029 | CB | ALA | D | 58 | 47.470 | 62.956 | 34.453 | 1.00 | 155.06 |
| 5030 | C | ALA | D | 58 | 48.976 | 63.508 | 32.559 | 1.00 | 180.25 |
| 5031 | O | ALA | D | 58 | 48.587 | 62.620 | 31.794 | 1.00 | 180.25 |
| 5032 | N | LYS | D | 59 | 50.209 | 64.009 | 32.531 | 1.00 | 167.78 |
| 5033 | CA | LYS | D | 59 | 51.224 | 63.558 | 31.585 | 1.00 | 167.78 |
| 5034 | CB | LYS | D | 59 | 51.992 | 64.761 | 31.017 | 1.00 | 249.69 |
| 5035 | CG | LYS | D | 59 | 51.110 | 65.772 | 30.290 | 1.00 | 249.69 |
| 5036 | CD | LYS | D | 59 | 51.889 | 67.003 | 29.845 | 1.00 | 249.69 |
| 5037 | CE | LYS | D | 59 | 50.965 | 68.013 | 29.165 | 1.00 | 249.69 |
| 5038 | NZ | LYS | D | 59 | 51.690 | 69.228 | 28.694 | 1.00 | 249.69 |
| 5039 | C | LYS | D | 59 | 52.159 | 62.654 | 32.356 | 1.00 | 167.78 |
| 5040 | O | LYS | D | 59 | 52.494 | 62.936 | 33.500 | 1.00 | 167.78 |
| 5041 | N | PHE | D | 60 | 52.566 | 61.564 | 31.727 | 1.00 | 220.31 |
| 5042 | CA | PHE | D | 60 | 53.457 | 60.608 | 32.364 | 1.00 | 220.31 |
| 5043 | CB | PHE | D | 60 | 54.062 | 59.699 | 31.294 | 1.00 | 243.71 |
| 5044 | CG | PHE | D | 60 | 53.053 | 58.843 | 30.590 | 1.00 | 243.71 |
| 5045 | CD1 | PHE | D | 60 | 53.280 | 58.409 | 29.293 | 1.00 | 243.71 |
| 5046 | CD2 | PHE | D | 60 | 51.881 | 58.454 | 31.232 | 1.00 | 243.71 |
| 5047 | CE1 | PHE | D | 60 | 52.357 | 57.600 | 28.639 | 1.00 | 243.71 |
| 5048 | CE2 | PHE | D | 60 | 50.954 | 57.647 | 30.589 | 1.00 | 243.71 |
| 5049 | CZ | PHE | D | 60 | 51.194 | 57.217 | 29.287 | 1.00 | 243.71 |
| 5050 | C | PHE | D | 60 | 54.571 | 61.240 | 33.202 | 1.00 | 220.31 |
| 5051 | O | PHE | D | 60 | 55.007 | 60.671 | 34.207 | 1.00 | 220.31 |
| 5052 | N | GLU | D | 61 | 55.025 | 62.417 | 32.789 | 1.00 | 201.05 |
| 5053 | CA | GLU | D | 61 | 56.101 | 63.125 | 33.487 | 1.00 | 201.05 |
| 5054 | CB | GLU | D | 61 | 56.545 | 64.332 | 32.653 | 1.00 | 249.69 |
| 5055 | CG | GLU | D | 61 | 57.065 | 63.987 | 31.251 | 1.00 | 249.69 |
| 5056 | CD | GLU | D | 61 | 56.045 | 63.244 | 30.385 | 1.00 | 249.69 |
| 5057 | OE1 | GLU | D | 61 | 54.897 | 63.728 | 30.245 | 1.00 | 249.69 |
| 5058 | OE2 | GLU | D | 61 | 56.395 | 62.176 | 29.835 | 1.00 | 249.69 |
| 5059 | C | GLU | D | 61 | 55.671 | 63.588 | 34.884 | 1.00 | 201.05 |
| 5060 | O | GLU | D | 61 | 56.512 | 63.803 | 35.757 | 1.00 | 201.05 |
| 5061 | N | ASP | D | 62 | 54.359 | 63.735 | 35.086 | 1.00 | 185.73 |
| 5062 | CA | ASP | D | 62 | 53.815 | 64.165 | 36.371 | 1.00 | 185.73 |
| 5063 | CB | ASP | D | 62 | 52.334 | 64.502 | 36.245 | 1.00 | 180.28 |
| 5064 | CG | ASP | D | 62 | 52.063 | 65.480 | 35.132 | 1.00 | 180.28 |
| 5065 | OD1 | ASP | D | 62 | 52.924 | 66.360 | 34.882 | 1.00 | 180.28 |
| 5066 | OD2 | ASP | D | 62 | 50.985 | 65.379 | 34.514 | 1.00 | 180.28 |
| 5067 | C | ASP | D | 62 | 53.982 | 63.078 | 37.414 | 1.00 | 185.73 |
| 5068 | O | ASP | D | 62 | 53.979 | 63.353 | 38.606 | 1.00 | 185.73 |
| 5069 | N | SER | D | 63 | 54.106 | 61.837 | 36.960 | 1.00 | 159.27 |
| 5070 | CA | SER | D | 63 | 54.292 | 60.711 | 37.864 | 1.00 | 159.27 |
| 5071 | CB | SER | D | 63 | 54.380 | 59.390 | 37.086 | 1.00 | 168.15 |
| 5072 | OG | SER | D | 63 | 53.226 | 59.160 | 36.318 | 1.00 | 168.15 |
| 5073 | C | SER | D | 63 | 55.608 | 60.949 | 38.595 | 1.00 | 159.27 |
| 5074 | O | SER | D | 63 | 56.640 | 61.192 | 37.968 | 1.00 | 159.27 |
| 5075 | N | GLY | D | 64 | 55.584 | 60.889 | 39.918 | 1.00 | 167.46 |
| 5076 | CA | GLY | D | 64 | 56.818 | 61.106 | 40.647 | 1.00 | 167.46 |
| 5077 | C | GLY | D | 64 | 56.687 | 61.299 | 42.144 | 1.00 | 167.46 |
| 5078 | O | GLY | D | 64 | 55.643 | 61.035 | 42.741 | 1.00 | 167.46 |
| 5079 | N | GLU | D | 65 | 57.772 | 61.788 | 42.737 | 1.00 | 249.05 |
| 5080 | CA | GLU | D | 65 | 57.882 | 62.037 | 44.170 | 1.00 | 249.05 |
| 5081 | CB | GLU | D | 65 | 59.223 | 61.476 | 44.640 | 1.00 | 248.88 |
| 5082 | CG | GLU | D | 65 | 59.604 | 61.791 | 46.061 | 1.00 | 248.88 |
| 5083 | CD | GLU | D | 65 | 61.083 | 61.571 | 46.298 | 1.00 | 248.88 |
| 5084 | OE1 | GLU | D | 65 | 61.892 | 62.265 | 45.646 | 1.00 | 248.88 |
| 5085 | OE2 | GLU | D | 65 | 61.439 | 60.704 | 47.125 | 1.00 | 248.88 |
| 5086 | C | GLU | D | 65 | 57.794 | 63.533 | 44.486 | 1.00 | 249.05 |
| 5087 | O | GLU | D | 65 | 58.598 | 64.318 | 43.989 | 1.00 | 249.05 |
| 5088 | N | TYR | D | 66 | 56.828 | 63.924 | 45.319 | 1.00 | 212.16 |
| 5089 | CA | TYR | D | 66 | 56.652 | 65.335 | 45.686 | 1.00 | 212.16 |
| 5090 | CB | TYR | D | 66 | 55.264 | 65.835 | 45.288 | 1.00 | 195.47 |
| 5091 | CG | TYR | D | 66 | 54.953 | 65.854 | 43.813 | 1.00 | 195.47 |
| 5092 | CD1 | TYR | D | 66 | 54.636 | 64.683 | 43.131 | 1.00 | 195.47 |
| 5093 | CE1 | TYR | D | 66 | 54.241 | 64.715 | 41.795 | 1.00 | 195.47 |
| 5094 | CD2 | TYR | D | 66 | 54.885 | 67.060 | 43.118 | 1.00 | 195.47 |
| 5095 | CE2 | TYR | D | 66 | 54.493 | 67.105 | 41.789 | 1.00 | 195.47 |
| 5096 | CZ | TYR | D | 66 | 54.169 | 65.932 | 41.131 | 1.00 | 195.47 |
| 5097 | OH | TYR | D | 66 | 53.738 | 65.982 | 39.822 | 1.00 | 195.47 |
| 5098 | C | TYR | D | 66 | 56.819 | 65.617 | 47.183 | 1.00 | 212.16 |
| 5099 | O | TYR | D | 66 | 56.894 | 64.694 | 47.993 | 1.00 | 212.16 |
| 5100 | N | LYS | D | 67 | 56.848 | 66.906 | 47.534 | 1.00 | 190.15 |
| 5101 | CA | LYS | D | 67 | 56.984 | 67.355 | 48.926 | 1.00 | 190.15 |
| 5102 | CB | LYS | D | 67 | 58.310 | 66.886 | 49.512 | 1.00 | 181.22 |
| 5103 | CG | LYS | D | 67 | 59.513 | 67.251 | 48.669 | 1.00 | 181.22 |
| 5104 | CD | LYS | D | 67 | 60.788 | 66.691 | 49.285 | 1.00 | 181.22 |
| 5105 | CE | LYS | D | 67 | 61.948 | 66.688 | 48.285 | 1.00 | 181.22 |
| 5106 | NZ | LYS | D | 67 | 63.216 | 66.167 | 48.871 | 1.00 | 181.22 |
| 5107 | C | LYS | D | 67 | 56.878 | 68.876 | 49.119 | 1.00 | 190.15 |
| 5108 | O | LYS | D | 67 | 57.155 | 69.660 | 48.209 | 1.00 | 190.15 |
| 5109 | N | CYS | D | 68 | 56.473 | 69.282 | 50.320 | 1.00 | 199.06 |
| 5110 | CA | CYS | D | 68 | 56.346 | 70.695 | 50.654 | 1.00 | 199.06 |
| 5111 | C | CYS | D | 68 | 57.039 | 71.004 | 51.975 | 1.00 | 199.06 |
| 5112 | O | CYS | D | 68 | 57.153 | 70.155 | 52.861 | 1.00 | 199.06 |
| 5113 | CB | CYS | D | 68 | 54.871 | 71.135 | 50.708 | 1.00 | 219.86 |
| 5114 | SG | CYS | D | 68 | 53.830 | 70.461 | 52.050 | 1.00 | 219.86 |
| 5115 | N | GLN | D | 69 | 57.505 | 72.240 | 52.083 | 1.00 | 249.17 |
| 5116 | CA | GLN | D | 69 | 58.212 | 72.728 | 53.257 | 1.00 | 249.17 |
| 5117 | CB | GLN | D | 69 | 59.714 | 72.495 | 53.072 | 1.00 | 249.69 |
| 5118 | CG | GLN | D | 69 | 60.606 | 73.364 | 53.942 | 1.00 | 249.69 |
| 5119 | CD | GLN | D | 69 | 62.082 | 73.199 | 53.613 | 1.00 | 249.69 |
| 5120 | OE1 | GLN | D | 69 | 62.491 | 73.322 | 52.452 | 1.00 | 249.69 |
| 5121 | NE2 | GLN | D | 69 | 62.892 | 72.925 | 54.634 | 1.00 | 249.69 |
| 5122 | C | GLN | D | 69 | 57.925 | 74.222 | 53.407 | 1.00 | 249.17 |
| 5123 | O | GLN | D | 69 | 57.726 | 74.927 | 52.418 | 1.00 | 249.17 |
| 5124 | N | HIS | D | 70 | 57.896 | 74.706 | 54.642 | 1.00 | 249.69 |
| 5125 | CA | HIS | D | 70 | 57.642 | 76.122 | 54.874 | 1.00 | 249.69 |
| 5126 | CB | HIS | D | 70 | 56.693 | 76.309 | 56.060 | 1.00 | 249.61 |
| 5127 | CG | HIS | D | 70 | 55.290 | 75.881 | 55.773 | 1.00 | 249.61 |
| 5128 | CD2 | HIS | D | 70 | 54.464 | 75.035 | 56.426 | 1.00 | 249.61 |
| 5129 | ND1 | HIS | D | 70 | 54.583 | 76.348 | 54.683 | 1.00 | 249.61 |
| 5130 | CE1 | HIS | D | 70 | 53.382 | 75.805 | 54.680 | 1.00 | 249.61 |
| 5131 | NE2 | HIS | D | 70 | 53.280 | 75.003 | 55.727 | 1.00 | 249.61 |
| 5132 | C | HIS | D | 70 | 58.936 | 76.887 | 55.115 | 1.00 | 249.69 |
| 5133 | O | HIS | D | 70 | 60.031 | 76.352 | 54.920 | 1.00 | 249.69 |
| 5134 | N | GLN | D | 71 | 58.803 | 78.140 | 55.540 | 1.00 | 249.69 |
| 5135 | CA | GLN | D | 71 | 59.955 | 79.008 | 55.802 | 1.00 | 249.69 |
| 5136 | CB | GLN | D | 71 | 59.459 | 80.374 | 56.307 | 1.00 | 249.69 |
| 5137 | CG | GLN | D | 71 | 60.461 | 81.539 | 56.197 | 1.00 | 249.69 |
| 5138 | CD | GLN | D | 71 | 60.863 | 81.861 | 54.754 | 1.00 | 249.69 |
| 5139 | OE1 | GLN | D | 71 | 60.016 | 81.976 | 53.858 | 1.00 | 249.69 |
| 5140 | NE2 | GLN | D | 71 | 62.163 | 82.021 | 54.530 | 1.00 | 249.69 |
| 5141 | C | GLN | D | 71 | 60.929 | 78.392 | 56.816 | 1.00 | 249.69 |
| 5142 | O | GLN | D | 71 | 62.143 | 78.352 | 56.581 | 1.00 | 249.69 |
| 5143 | N | GLN | D | 72 | 60.389 | 77.905 | 57.932 | 1.00 | 249.69 |
| 5144 | CA | GLN | D | 72 | 61.203 | 77.301 | 58.984 | 1.00 | 249.69 |
| 5145 | CB | GLN | D | 72 | 61.267 | 78.257 | 60.181 | 1.00 | 247.95 |
| 5146 | CG | GLN | D | 72 | 62.117 | 77.792 | 61.354 | 1.00 | 247.95 |
| 5147 | CD | GLN | D | 72 | 61.994 | 78.719 | 62.551 | 1.00 | 247.95 |
| 5148 | OE1 | GLN | D | 72 | 62.253 | 79.917 | 62.449 | 1.00 | 247.95 |
| 5149 | NE2 | GLN | D | 72 | 61.591 | 78.168 | 63.693 | 1.00 | 247.95 |
| 5150 | C | GLN | D | 72 | 60.624 | 75.947 | 59.409 | 1.00 | 249.69 |
| 5151 | O | GLN | D | 72 | 60.335 | 75.721 | 60.584 | 1.00 | 249.69 |
| 5152 | N | VAL | D | 73 | 60.449 | 75.052 | 58.443 | 1.00 | 248.81 |
| 5153 | CA | VAL | D | 73 | 59.911 | 73.726 | 58.720 | 1.00 | 248.81 |
| 5154 | CB | VAL | D | 73 | 58.396 | 73.640 | 58.398 | 1.00 | 224.12 |
| 5155 | CG1 | VAL | D | 73 | 57.822 | 72.365 | 58.991 | 1.00 | 224.12 |
| 5156 | CG2 | VAL | D | 73 | 57.664 | 74.863 | 58.936 | 1.00 | 224.12 |
| 5157 | C | VAL | D | 73 | 60.641 | 72.704 | 57.858 | 1.00 | 248.81 |
| 5158 | O | VAL | D | 73 | 60.991 | 72.985 | 56.717 | 1.00 | 248.81 |
| 5159 | N | ASN | D | 74 | 60.869 | 71.519 | 58.404 | 1.00 | 237.91 |
| 5160 | CA | ASN | D | 74 | 61.552 | 70.475 | 57.660 | 1.00 | 237.91 |
| 5161 | CB | ASN | D | 74 | 62.098 | 69.419 | 58.631 | 1.00 | 218.21 |
| 5162 | CG | ASN | D | 74 | 63.003 | 70.022 | 59.699 | 1.00 | 218.21 |
| 5163 | OD1 | ASN | D | 74 | 63.818 | 70.893 | 59.398 | 1.00 | 218.21 |
| 5164 | ND2 | ASN | D | 74 | 62.865 | 69.553 | 60.940 | 1.00 | 218.21 |
| 5165 | C | ASN | D | 74 | 60.595 | 69.846 | 56.635 | 1.00 | 237.91 |
| 5166 | O | ASN | D | 74 | 59.477 | 69.450 | 56.973 | 1.00 | 237.91 |
| 5167 | N | GLU | D | 75 | 61.042 | 69.772 | 55.381 | 1.00 | 249.69 |
| 5168 | CA | GLU | D | 75 | 60.252 | 69.219 | 54.276 | 1.00 | 249.69 |
| 5169 | CB | GLU | D | 75 | 61.161 | 68.971 | 53.065 | 1.00 | 244.63 |
| 5170 | CG | GLU | D | 75 | 62.563 | 68.479 | 53.417 | 1.00 | 244.63 |
| 5171 | CD | GLU | D | 75 | 63.503 | 68.477 | 52.223 | 1.00 | 244.63 |
| 5172 | OE1 | GLU | D | 75 | 63.579 | 69.508 | 51.523 | 1.00 | 244.63 |
| 5173 | OE2 | GLU | D | 75 | 64.172 | 67.448 | 51.989 | 1.00 | 244.63 |
| 5174 | C | GLU | D | 75 | 59.463 | 67.954 | 54.609 | 1.00 | 249.69 |
| 5175 | O | GLU | D | 75 | 59.947 | 67.066 | 55.313 | 1.00 | 249.69 |
| 5176 | N | SER | D | 76 | 58.244 | 67.886 | 54.080 | 1.00 | 245.54 |
| 5177 | CA | SER | D | 76 | 57.338 | 66.759 | 54.313 | 1.00 | 245.54 |
| 5178 | CB | SER | D | 76 | 55.952 | 67.070 | 53.739 | 1.00 | 192.35 |
| 5179 | OG | SER | D | 76 | 55.961 | 66.977 | 52.328 | 1.00 | 192.35 |
| 5180 | C | SER | D | 76 | 57.811 | 65.437 | 53.721 | 1.00 | 245.54 |
| 5181 | O | SER | D | 76 | 58.617 | 65.413 | 52.791 | 1.00 | 245.54 |
| 5182 | N | GLU | D | 77 | 57.289 | 64.340 | 54.268 | 1.00 | 239.60 |
| 5183 | CA | GLU | D | 77 | 57.628 | 63.013 | 53.788 | 1.00 | 239.60 |
| 5184 | CB | GLU | D | 77 | 56.972 | 61.938 | 54.663 | 1.00 | 249.69 |
| 5185 | CG | GLU | D | 77 | 57.497 | 61.895 | 56.089 | 1.00 | 249.69 |
| 5186 | CD | GLU | D | 77 | 58.969 | 61.533 | 56.157 | 1.00 | 249.69 |
| 5187 | OE1 | GLU | D | 77 | 59.621 | 61.453 | 55.090 | 1.00 | 249.69 |
| 5188 | OE2 | GLU | D | 77 | 59.476 | 61.333 | 57.282 | 1.00 | 249.69 |
| 5189 | C | GLU | D | 77 | 57.106 | 62.902 | 52.366 | 1.00 | 239.60 |
| 5190 | O | GLU | D | 77 | 55.898 | 62.905 | 52.139 | 1.00 | 239.60 |
| 5191 | N | PRO | D | 78 | 58.019 | 62.800 | 51.386 | 1.00 | 190.36 |
| 5192 | CD | PRO | D | 78 | 59.457 | 62.543 | 51.572 | 1.00 | 231.46 |
| 5193 | CA | PRO | D | 78 | 57.642 | 62.695 | 49.969 | 1.00 | 190.36 |
| 5194 | CB | PRO | D | 78 | 58.919 | 62.159 | 49.320 | 1.00 | 231.46 |
| 5195 | CG | PRO | D | 78 | 60.002 | 62.732 | 50.179 | 1.00 | 231.46 |
| 5196 | C | PRO | D | 78 | 56.456 | 61.764 | 49.731 | 1.00 | 190.36 |
| 5197 | O | PRO | D | 78 | 56.163 | 60.897 | 50.553 | 1.00 | 190.36 |
| 5198 | N | VAL | D | 79 | 55.760 | 61.969 | 48.620 | 1.00 | 182.77 |
| 5199 | CA | VAL | D | 79 | 54.639 | 61.112 | 48.261 | 1.00 | 182.77 |
| 5200 | CB | VAL | D | 79 | 53.272 | 61.804 | 48.426 | 1.00 | 114.23 |
| 5201 | CG1 | VAL | D | 79 | 52.173 | 60.961 | 47.770 | 1.00 | 114.23 |
| 5202 | CG2 | VAL | D | 79 | 52.957 | 61.977 | 49.909 | 1.00 | 114.23 |
| 5203 | C | VAL | D | 79 | 54.845 | 60.799 | 46.800 | 1.00 | 182.77 |
| 5204 | O | VAL | D | 79 | 55.292 | 61.663 | 46.043 | 1.00 | 182.77 |
| 5205 | N | TYR | D | 80 | 54.538 | 59.570 | 46.397 | 1.00 | 172.05 |
| 5206 | CA | TYR | D | 80 | 54.726 | 59.204 | 45.004 | 1.00 | 172.05 |
| 5207 | CB | TYR | D | 80 | 55.475 | 57.870 | 44.888 | 1.00 | 249.30 |
| 5208 | CG | TYR | D | 80 | 56.087 | 57.651 | 43.518 | 1.00 | 249.30 |
| 5209 | CD1 | TYR | D | 80 | 57.327 | 58.197 | 43.191 | 1.00 | 249.30 |
| 5210 | CE1 | TYR | D | 80 | 57.868 | 58.042 | 41.917 | 1.00 | 249.30 |
| 5211 | CD2 | TYR | D | 80 | 55.403 | 56.940 | 42.531 | 1.00 | 249.30 |
| 5212 | CE2 | TYR | D | 80 | 55.937 | 56.780 | 41.254 | 1.00 | 249.30 |
| 5213 | CZ | TYR | D | 80 | 57.167 | 57.335 | 40.956 | 1.00 | 249.30 |
| 5214 | OH | TYR | D | 80 | 57.689 | 57.191 | 39.696 | 1.00 | 249.30 |
| 5215 | C | TYR | D | 80 | 53.407 | 59.120 | 44.238 | 1.00 | 172.05 |
| 5216 | O | TYR | D | 80 | 52.419 | 58.550 | 44.701 | 1.00 | 172.05 |
| 5217 | N | LEU | D | 81 | 53.407 | 59.702 | 43.054 | 1.00 | 138.80 |
| 5218 | CA | LEU | D | 81 | 52.239 | 59.681 | 42.207 | 1.00 | 138.80 |
| 5219 | CB | LEU | D | 81 | 51.837 | 61.099 | 41.821 | 1.00 | 163.84 |
| 5220 | CG | LEU | D | 81 | 50.683 | 61.126 | 40.812 | 1.00 | 163.84 |
| 5221 | CD1 | LEU | D | 81 | 49.462 | 60.512 | 41.463 | 1.00 | 163.84 |
| 5222 | CD2 | LEU | D | 81 | 50.394 | 62.539 | 40.357 | 1.00 | 163.84 |
| 5223 | C | LEU | D | 81 | 52.560 | 58.907 | 40.932 | 1.00 | 138.80 |
| 5224 | O | LEU | D | 81 | 53.554 | 59.196 | 40.251 | 1.00 | 138.80 |
| 5225 | N | GLU | D | 82 | 51.728 | 57.924 | 40.598 | 1.00 | 140.95 |
| 5226 | CA | GLU | D | 82 | 51.964 | 57.149 | 39.380 | 1.00 | 140.95 |
| 5227 | CB | GLU | D | 82 | 52.148 | 55.662 | 39.716 | 1.00 | 249.69 |
| 5228 | CG | GLU | D | 82 | 52.985 | 54.915 | 38.685 | 1.00 | 249.69 |
| 5229 | CD | GLU | D | 82 | 53.177 | 53.447 | 39.028 | 1.00 | 249.69 |
| 5230 | OE1 | GLU | D | 82 | 53.282 | 53.118 | 40.233 | 1.00 | 249.69 |
| 5231 | OE2 | GLU | D | 82 | 53.238 | 52.623 | 38.088 | 1.00 | 249.69 |
| 5232 | C | GLU | D | 82 | 50.798 | 57.324 | 38.415 | 1.00 | 140.95 |
| 5233 | O | GLU | D | 82 | 49.629 | 57.287 | 38.824 | 1.00 | 140.95 |
| 5234 | N | VAL | D | 83 | 51.121 | 57.533 | 37.139 | 1.00 | 204.94 |
| 5235 | CA | VAL | D | 83 | 50.098 | 57.704 | 36.106 | 1.00 | 204.94 |
| 5236 | CB | VAL | D | 83 | 50.307 | 58.985 | 35.282 | 1.00 | 154.41 |
| 5237 | CG1 | VAL | D | 83 | 49.194 | 59.119 | 34.248 | 1.00 | 154.41 |
| 5238 | CG2 | VAL | D | 83 | 50.333 | 60.203 | 36.207 | 1.00 | 154.41 |
| 5239 | C | VAL | D | 83 | 50.072 | 56.510 | 35.159 | 1.00 | 204.94 |
| 5240 | O | VAL | D | 83 | 51.125 | 55.962 | 34.781 | 1.00 | 204.94 |
| 5241 | N | PHE | D | 84 | 48.858 | 56.137 | 34.755 | 1.00 | 140.70 |
| 5242 | CA | PHE | D | 84 | 48.655 | 54.961 | 33.916 | 1.00 | 140.70 |
| 5243 | CB | PHE | D | 84 | 48.013 | 53.843 | 34.738 | 1.00 | 172.63 |
| 5244 | CG | PHE | D | 84 | 48.828 | 53.387 | 35.905 | 1.00 | 172.63 |
| 5245 | CD1 | PHE | D | 84 | 48.823 | 54.095 | 37.102 | 1.00 | 172.63 |
| 5246 | CD2 | PHE | D | 84 | 49.579 | 52.229 | 35.815 | 1.00 | 172.63 |
| 5247 | CE1 | PHE | D | 84 | 49.553 | 53.648 | 38.187 | 1.00 | 172.63 |
| 5248 | CE2 | PHE | D | 84 | 50.310 | 51.776 | 36.892 | 1.00 | 172.63 |
| 5249 | CZ | PHE | D | 84 | 50.298 | 52.483 | 38.084 | 1.00 | 172.63 |
| 5250 | C | PHE | D | 84 | 47.811 | 55.075 | 32.669 | 1.00 | 140.70 |
| 5251 | O | PHE | D | 84 | 46.952 | 55.941 | 32.546 | 1.00 | 140.70 |
| 5252 | N | SER | D | 85 | 48.057 | 54.127 | 31.773 | 1.00 | 187.78 |
| 5253 | CA | SER | D | 85 | 47.318 | 53.999 | 30.534 | 1.00 | 187.78 |
| 5254 | CB | SER | D | 85 | 48.163 | 54.364 | 29.322 | 1.00 | 228.86 |
| 5255 | OG | SER | D | 85 | 47.394 | 54.223 | 28.141 | 1.00 | 228.86 |
| 5256 | C | SER | D | 85 | 46.957 | 52.522 | 30.461 | 1.00 | 187.78 |
| 5257 | O | SER | D | 85 | 47.841 | 51.663 | 30.344 | 1.00 | 187.78 |
| 5258 | N | ASP | D | 86 | 45.657 | 52.235 | 30.551 | 1.00 | 145.83 |
| 5259 | CA | ASP | D | 86 | 45.141 | 50.864 | 30.508 | 1.00 | 145.83 |
| 5260 | CB | ASP | D | 86 | 45.692 | 50.069 | 31.690 | 1.00 | 155.62 |
| 5261 | CG | ASP | D | 86 | 45.997 | 48.650 | 31.328 | 1.00 | 155.62 |
| 5262 | OD1 | ASP | D | 86 | 45.107 | 47.971 | 30.755 | 1.00 | 155.62 |
| 5263 | OD2 | ASP | D | 86 | 47.133 | 48.214 | 31.610 | 1.00 | 155.62 |
| 5264 | C | ASP | D | 86 | 43.621 | 50.885 | 30.580 | 1.00 | 145.83 |
| 5265 | O | ASP | D | 86 | 43.019 | 51.914 | 30.878 | 1.00 | 145.83 |
| 5266 | N | TRP | D | 87 | 42.997 | 49.749 | 30.315 | 1.00 | 152.62 |
| 5267 | CA | TRP | D | 87 | 41.544 | 49.688 | 30.387 | 1.00 | 152.62 |
| 5268 | CB | TRP | D | 87 | 41.038 | 48.369 | 29.817 | 1.00 | 249.69 |
| 5269 | CG | TRP | D | 87 | 40.784 | 48.449 | 28.349 | 1.00 | 249.69 |
| 5270 | CD2 | TRP | D | 87 | 41.700 | 48.112 | 27.306 | 1.00 | 249.69 |
| 5271 | CE2 | TRP | D | 87 | 41.058 | 48.385 | 26.079 | 1.00 | 249.69 |
| 5272 | CE3 | TRP | D | 87 | 43.010 | 47.599 | 27.285 | 1.00 | 249.69 |
| 5273 | CD1 | TRP | D | 87 | 39.649 | 48.907 | 27.735 | 1.00 | 249.69 |
| 5274 | NE1 | TRP | D | 87 | 39.807 | 48.868 | 26.373 | 1.00 | 249.69 |
| 5275 | CZ2 | TRP | D | 87 | 41.674 | 48.164 | 24.845 | 1.00 | 249.69 |
| 5276 | CZ3 | TRP | D | 87 | 43.623 | 47.377 | 26.056 | 1.00 | 249.69 |
| 5277 | CH2 | TRP | D | 87 | 42.955 | 47.666 | 24.854 | 1.00 | 249.69 |
| 5278 | C | TRP | D | 87 | 41.058 | 49.854 | 31.821 | 1.00 | 152.62 |
| 5279 | O | TRP | D | 87 | 40.220 | 50.708 | 32.092 | 1.00 | 152.62 |
| 5280 | N | LEU | D | 88 | 41.578 | 49.041 | 32.739 | 1.00 | 136.73 |
| 5281 | CA | LEU | D | 88 | 41.190 | 49.161 | 34.137 | 1.00 | 136.73 |
| 5282 | CB | LEU | D | 88 | 40.415 | 47.933 | 34.574 | 1.00 | 120.19 |
| 5283 | CG | LEU | D | 88 | 39.068 | 47.722 | 33.876 | 1.00 | 120.19 |
| 5284 | CD1 | LEU | D | 88 | 38.314 | 46.521 | 34.481 | 1.00 | 120.19 |
| 5285 | CD2 | LEU | D | 88 | 38.232 | 48.972 | 34.012 | 1.00 | 120.19 |
| 5286 | C | LEU | D | 88 | 42.405 | 49.364 | 35.043 | 1.00 | 136.73 |
| 5287 | O | LEU | D | 88 | 43.486 | 48.794 | 34.806 | 1.00 | 136.73 |
| 5288 | N | LEU | D | 89 | 42.238 | 50.206 | 36.063 | 1.00 | 129.46 |
| 5289 | CA | LEU | D | 89 | 43.315 | 50.483 | 37.021 | 1.00 | 129.46 |
| 5290 | CB | LEU | D | 89 | 43.867 | 51.888 | 36.838 | 1.00 | 166.27 |
| 5291 | CG | LEU | D | 89 | 44.935 | 52.257 | 37.864 | 1.00 | 166.27 |
| 5292 | CD1 | LEU | D | 89 | 46.034 | 51.209 | 37.875 | 1.00 | 166.27 |
| 5293 | CD2 | LEU | D | 89 | 45.502 | 53.621 | 37.532 | 1.00 | 166.27 |
| 5294 | C | LEU | D | 89 | 42.751 | 50.361 | 38.425 | 1.00 | 129.46 |
| 5295 | O | LEU | D | 89 | 41.706 | 50.925 | 38.731 | 1.00 | 129.46 |
| 5296 | N | LEU | D | 90 | 43.435 | 49.611 | 39.278 | 1.00 | 149.29 |
| 5297 | CA | LEU | D | 90 | 42.966 | 49.415 | 40.640 | 1.00 | 149.29 |
| 5298 | CB | LEU | D | 90 | 43.433 | 48.070 | 41.171 | 1.00 | 119.67 |
| 5299 | CG | LEU | D | 90 | 43.122 | 47.859 | 42.640 | 1.00 | 119.67 |
| 5300 | CD1 | LEU | D | 90 | 41.612 | 47.911 | 42.843 | 1.00 | 119.67 |
| 5301 | CD2 | LEU | D | 90 | 43.686 | 46.531 | 43.108 | 1.00 | 119.67 |
| 5302 | C | LEU | D | 90 | 43.486 | 50.508 | 41.543 | 1.00 | 149.29 |
| 5303 | O | LEU | D | 90 | 44.695 | 50.591 | 41.785 | 1.00 | 149.29 |
| 5304 | N | GLN | D | 91 | 42.577 | 51.336 | 42.059 | 1.00 | 104.53 |
| 5305 | CA | GLN | D | 91 | 42.981 | 52.439 | 42.946 | 1.00 | 104.53 |
| 5306 | CB | GLN | D | 91 | 42.241 | 53.716 | 42.566 | 1.00 | 160.34 |
| 5307 | CG | GLN | D | 91 | 42.495 | 54.188 | 41.147 | 1.00 | 160.34 |
| 5308 | CD | GLN | D | 91 | 41.751 | 55.470 | 40.843 | 1.00 | 160.34 |
| 5309 | OE1 | GLN | D | 91 | 40.527 | 55.541 | 40.995 | 1.00 | 160.34 |
| 5310 | NE2 | GLN | D | 91 | 42.485 | 56.493 | 40.412 | 1.00 | 160.34 |
| 5311 | C | GLN | D | 91 | 42.756 | 52.156 | 44.424 | 1.00 | 104.53 |
| 5312 | O | GLN | D | 91 | 41.691 | 51.645 | 44.823 | 1.00 | 104.53 |
| 5313 | N | ALA | D | 92 | 43.762 | 52.506 | 45.227 | 1.00 | 107.84 |
| 5314 | CA | ALA | D | 92 | 43.673 | 52.285 | 46.657 | 1.00 | 107.84 |
| 5315 | CB | ALA | D | 92 | 44.731 | 51.297 | 47.102 | 1.00 | 189.20 |
| 5316 | C | ALA | D | 92 | 43.850 | 53.594 | 47.399 | 1.00 | 107.84 |
| 5317 | O | ALA | D | 92 | 44.683 | 54.436 | 46.987 | 1.00 | 107.84 |
| 5318 | N | SER | D | 93 | 43.062 | 53.754 | 48.477 | 1.00 | 115.75 |
| 5319 | CA | SER | D | 93 | 43.108 | 54.947 | 49.319 | 1.00 | 115.75 |
| 5320 | CB | SER | D | 93 | 42.212 | 54.756 | 50.553 | 1.00 | 115.29 |
| 5321 | OG | SER | D | 93 | 42.503 | 53.560 | 51.271 | 1.00 | 115.29 |
| 5322 | C | SER | D | 93 | 44.559 | 55.177 | 49.730 | 1.00 | 115.75 |
| 5323 | O | SER | D | 93 | 45.176 | 56.193 | 49.371 | 1.00 | 115.75 |
| 5324 | N | ALA | D | 94 | 45.094 | 54.214 | 50.476 | 1.00 | 146.78 |
| 5325 | CA | ALA | D | 94 | 46.481 | 54.242 | 50.924 | 1.00 | 146.78 |
| 5326 | CB | ALA | D | 94 | 46.552 | 54.536 | 52.412 | 1.00 | 207.94 |
| 5327 | C | ALA | D | 94 | 46.992 | 52.846 | 50.626 | 1.00 | 146.78 |
| 5328 | O | ALA | D | 94 | 46.194 | 51.905 | 50.566 | 1.00 | 146.78 |
| 5329 | N | GLU | D | 95 | 48.300 | 52.699 | 50.426 | 1.00 | 134.86 |
| 5330 | CA | GLU | D | 95 | 48.844 | 51.383 | 50.125 | 1.00 | 134.86 |
| 5331 | CB | GLU | D | 95 | 49.967 | 51.498 | 49.101 | 1.00 | 220.03 |
| 5332 | CG | GLU | D | 95 | 49.489 | 52.026 | 47.768 | 1.00 | 220.03 |
| 5333 | CD | GLU | D | 95 | 50.535 | 51.898 | 46.687 | 1.00 | 220.03 |
| 5334 | OE1 | GLU | D | 95 | 50.271 | 52.341 | 45.547 | 1.00 | 220.03 |
| 5335 | OE2 | GLU | D | 95 | 51.621 | 51.351 | 46.970 | 1.00 | 220.03 |
| 5336 | C | GLU | D | 95 | 49.335 | 50.662 | 51.376 | 1.00 | 134.86 |
| 5337 | O | GLU | D | 95 | 49.412 | 49.423 | 51.408 | 1.00 | 134.86 |
| 5338 | N | VAL | D | 96 | 49.655 | 51.439 | 52.407 | 1.00 | 128.32 |
| 5339 | CA | VAL | D | 96 | 50.122 | 50.876 | 53.668 | 1.00 | 128.32 |
| 5340 | CB | VAL | D | 96 | 51.561 | 51.292 | 53.949 | 1.00 | 128.30 |
| 5341 | CG1 | VAL | D | 96 | 52.157 | 50.377 | 55.003 | 1.00 | 128.30 |
| 5342 | CG2 | VAL | D | 96 | 52.372 | 51.260 | 52.675 | 1.00 | 128.30 |
| 5343 | C | VAL | D | 96 | 49.242 | 51.383 | 54.816 | 1.00 | 128.32 |
| 5344 | O | VAL | D | 96 | 49.010 | 52.588 | 54.932 | 1.00 | 128.32 |
| 5345 | N | VAL | D | 97 | 48.775 | 50.480 | 55.678 | 1.00 | 152.31 |
| 5346 | CA | VAL | D | 97 | 47.890 | 50.898 | 56.756 | 1.00 | 152.31 |
| 5347 | CB | VAL | D | 97 | 46.438 | 50.575 | 56.406 | 1.00 | 113.44 |
| 5348 | CG1 | VAL | D | 97 | 45.533 | 51.442 | 57.216 | 1.00 | 113.44 |
| 5349 | CG2 | VAL | D | 97 | 46.185 | 50.774 | 54.941 | 1.00 | 113.44 |
| 5350 | C | VAL | D | 97 | 48.135 | 50.330 | 58.152 | 1.00 | 152.31 |
| 5351 | O | VAL | D | 97 | 48.616 | 49.193 | 58.305 | 1.00 | 152.31 |
| 5352 | N | MET | D | 98 | 47.765 | 51.133 | 59.160 | 1.00 | 133.53 |
| 5353 | CA | MET | D | 98 | 47.880 | 50.784 | 60.590 | 1.00 | 133.53 |
| 5354 | CB | MET | D | 98 | 47.936 | 52.058 | 61.440 | 1.00 | 228.89 |
| 5355 | CG | MET | D | 98 | 49.145 | 52.951 | 61.220 | 1.00 | 228.89 |
| 5356 | SD | MET | D | 98 | 50.627 | 52.296 | 62.005 | 1.00 | 228.89 |
| 5357 | CE | MET | D | 98 | 50.300 | 52.679 | 63.720 | 1.00 | 228.89 |
| 5358 | C | MET | D | 98 | 46.651 | 49.985 | 61.010 | 1.00 | 133.53 |
| 5359 | O | MET | D | 98 | 45.535 | 50.470 | 60.868 | 1.00 | 133.53 |
| 5360 | N | GLU | D | 99 | 46.849 | 48.786 | 61.547 | 1.00 | 198.14 |
| 5361 | CA | GLU | D | 99 | 45.726 | 47.954 | 61.963 | 1.00 | 198.14 |
| 5362 | CB | GLU | D | 99 | 46.179 | 46.922 | 63.001 | 1.00 | 249.68 |
| 5363 | CG | GLU | D | 99 | 45.303 | 45.675 | 63.049 | 1.00 | 249.68 |
| 5364 | CD | GLU | D | 99 | 45.586 | 44.809 | 64.259 | 1.00 | 249.68 |
| 5365 | OE1 | GLU | D | 99 | 46.770 | 44.698 | 64.645 | 1.00 | 249.68 |
| 5366 | OE2 | GLU | D | 99 | 44.627 | 44.233 | 64.816 | 1.00 | 249.68 |
| 5367 | C | GLU | D | 99 | 44.614 | 48.812 | 62.563 | 1.00 | 198.14 |
| 5368 | O | GLU | D | 99 | 44.852 | 49.583 | 63.489 | 1.00 | 198.14 |
| 5369 | N | GLY | D | 100 | 43.402 | 48.692 | 62.031 | 1.00 | 166.74 |
| 5370 | CA | GLY | D | 100 | 42.296 | 49.467 | 62.569 | 1.00 | 166.74 |
| 5371 | C | GLY | D | 100 | 41.812 | 50.625 | 61.718 | 1.00 | 166.74 |
| 5372 | O | GLY | D | 100 | 40.670 | 51.053 | 61.861 | 1.00 | 166.74 |
| 5373 | N | GLN | D | 101 | 42.669 | 51.133 | 60.842 | 1.00 | 134.36 |
| 5374 | CA | GLN | D | 101 | 42.317 | 52.250 | 59.961 | 1.00 | 134.36 |
| 5375 | CB | GLN | D | 101 | 43.571 | 52.882 | 59.372 | 1.00 | 207.44 |
| 5376 | CG | GLN | D | 101 | 44.392 | 53.630 | 60.387 | 1.00 | 207.44 |
| 5377 | CD | GLN | D | 101 | 43.524 | 54.494 | 61.269 | 1.00 | 207.44 |
| 5378 | OE1 | GLN | D | 101 | 42.774 | 53.987 | 62.103 | 1.00 | 207.44 |
| 5379 | NE2 | GLN | D | 101 | 43.607 | 55.807 | 61.083 | 1.00 | 207.44 |
| 5380 | C | GLN | D | 101 | 41.345 | 51.862 | 58.841 | 1.00 | 134.36 |
| 5381 | O | GLN | D | 101 | 41.004 | 50.693 | 58.670 | 1.00 | 134.36 |
| 5382 | N | PRO | D | 102 | 40.894 | 52.871 | 58.051 | 1.00 | 115.73 |
| 5383 | CD | PRO | D | 102 | 40.996 | 54.307 | 58.285 | 1.00 | 176.17 |
| 5384 | CA | PRO | D | 102 | 39.973 | 52.569 | 56.946 | 1.00 | 115.73 |
| 5385 | CB | PRO | D | 102 | 39.138 | 53.843 | 56.890 | 1.00 | 176.17 |
| 5386 | CG | PRO | D | 102 | 40.181 | 54.884 | 57.136 | 1.00 | 176.17 |
| 5387 | C | PRO | D | 102 | 40.678 | 52.280 | 55.630 | 1.00 | 115.73 |
| 5388 | O | PRO | D | 102 | 41.771 | 52.810 | 55.342 | 1.00 | 115.73 |
| 5389 | N | LEU | D | 103 | 40.042 | 51.439 | 54.818 | 1.00 | 118.65 |
| 5390 | CA | LEU | D | 103 | 40.610 | 51.062 | 53.547 | 1.00 | 118.65 |
| 5391 | CB | LEU | D | 103 | 41.185 | 49.671 | 53.652 | 1.00 | 120.76 |
| 5392 | CG | LEU | D | 103 | 42.003 | 49.394 | 52.400 | 1.00 | 120.76 |
| 5393 | CD1 | LEU | D | 103 | 43.228 | 50.310 | 52.430 | 1.00 | 120.76 |
| 5394 | CD2 | LEU | D | 103 | 42.399 | 47.938 | 52.323 | 1.00 | 120.76 |
| 5395 | C | LEU | D | 103 | 39.597 | 51.065 | 52.413 | 1.00 | 118.65 |
| 5396 | O | LEU | D | 103 | 38.575 | 50.389 | 52.508 | 1.00 | 118.65 |
| 5397 | N | PHE | D | 104 | 39.875 | 51.798 | 51.332 | 1.00 | 129.85 |
| 5398 | CA | PHE | D | 104 | 38.955 | 51.830 | 50.187 | 1.00 | 129.85 |
| 5399 | CB | PHE | D | 104 | 38.327 | 53.199 | 50.024 | 1.00 | 234.23 |
| 5400 | CG | PHE | D | 104 | 37.655 | 53.699 | 51.249 | 1.00 | 234.23 |
| 5401 | CD1 | PHE | D | 104 | 38.402 | 54.218 | 52.300 | 1.00 | 234.23 |
| 5402 | CD2 | PHE | D | 104 | 36.273 | 53.651 | 51.365 | 1.00 | 234.23 |
| 5403 | CE1 | PHE | D | 104 | 37.779 | 54.688 | 53.457 | 1.00 | 234.23 |
| 5404 | CE2 | PHE | D | 104 | 35.638 | 54.116 | 52.517 | 1.00 | 234.23 |
| 5405 | CZ | PHE | D | 104 | 36.393 | 54.637 | 53.567 | 1.00 | 234.23 |
| 5406 | C | PHE | D | 104 | 39.651 | 51.480 | 48.881 | 1.00 | 129.85 |
| 5407 | O | PHE | D | 104 | 40.632 | 52.139 | 48.499 | 1.00 | 129.85 |
| 5408 | N | LEU | D | 105 | 39.152 | 50.446 | 48.198 | 1.00 | 126.08 |
| 5409 | CA | LEU | D | 105 | 39.725 | 50.039 | 46.914 | 1.00 | 126.08 |
| 5410 | CB | LEU | D | 105 | 40.031 | 48.543 | 46.910 | 1.00 | 130.08 |
| 5411 | CG | LEU | D | 105 | 41.013 | 48.106 | 47.993 | 1.00 | 130.08 |
| 5412 | CD1 | LEU | D | 105 | 41.358 | 46.640 | 47.803 | 1.00 | 130.08 |
| 5413 | CD2 | LEU | D | 105 | 42.266 | 48.976 | 47.919 | 1.00 | 130.08 |
| 5414 | C | LEU | D | 105 | 38.719 | 50.360 | 45.832 | 1.00 | 126.08 |
| 5415 | O | LEU | D | 105 | 37.510 | 50.328 | 46.061 | 1.00 | 126.08 |
| 5416 | N | ARG | D | 106 | 39.203 | 50.658 | 44.641 | 1.00 | 133.86 |
| 5417 | CA | ARG | D | 106 | 38.288 | 51.013 | 43.581 | 1.00 | 133.86 |
| 5418 | CB | ARG | D | 106 | 38.213 | 52.537 | 43.522 | 1.00 | 170.25 |
| 5419 | CG | ARG | D | 106 | 37.267 | 53.090 | 42.509 | 1.00 | 170.25 |
| 5420 | CD | ARG | D | 106 | 37.416 | 54.594 | 42.401 | 1.00 | 170.25 |
| 5421 | NE | ARG | D | 106 | 36.634 | 55.086 | 41.272 | 1.00 | 170.25 |
| 5422 | CZ | ARG | D | 106 | 36.946 | 56.155 | 40.553 | 1.00 | 170.25 |
| 5423 | NH1 | ARG | D | 106 | 38.031 | 56.855 | 40.841 | 1.00 | 170.25 |
| 5424 | NH2 | ARG | D | 106 | 36.181 | 56.503 | 39.529 | 1.00 | 170.25 |
| 5425 | C | ARG | D | 106 | 38.732 | 50.439 | 42.239 | 1.00 | 133.86 |
| 5426 | O | ARG | D | 106 | 39.882 | 50.624 | 41.824 | 1.00 | 133.86 |
| 5427 | N | CYS | D | 107 | 37.833 | 49.718 | 41.572 | 1.00 | 163.35 |
| 5428 | CA | CYS | D | 107 | 38.144 | 49.158 | 40.256 | 1.00 | 163.35 |
| 5429 | C | CYS | D | 107 | 37.800 | 50.290 | 39.316 | 1.00 | 163.35 |
| 5430 | O | CYS | D | 107 | 36.621 | 50.528 | 39.043 | 1.00 | 163.35 |
| 5431 | CB | CYS | D | 107 | 37.250 | 47.960 | 39.954 | 1.00 | 164.16 |
| 5432 | SG | CYS | D | 107 | 37.777 | 46.964 | 38.529 | 1.00 | 164.16 |
| 5433 | N | HIS | D | 108 | 38.824 | 50.991 | 38.834 | 1.00 | 196.08 |
| 5434 | CA | HIS | D | 108 | 38.626 | 52.153 | 37.962 | 1.00 | 196.08 |
| 5435 | CB | HIS | D | 108 | 39.641 | 53.252 | 38.313 | 1.00 | 199.66 |
| 5436 | CG | HIS | D | 108 | 39.354 | 54.579 | 37.678 | 1.00 | 199.66 |
| 5437 | CD2 | HIS | D | 108 | 40.151 | 55.424 | 36.978 | 1.00 | 199.66 |
| 5438 | ND1 | HIS | D | 108 | 38.135 | 55.214 | 37.796 | 1.00 | 199.66 |
| 5439 | CE1 | HIS | D | 108 | 38.197 | 56.390 | 37.202 | 1.00 | 199.66 |
| 5440 | NE2 | HIS | D | 108 | 39.411 | 56.543 | 36.699 | 1.00 | 199.66 |
| 5441 | C | HIS | D | 108 | 38.691 | 51.885 | 36.473 | 1.00 | 196.08 |
| 5442 | O | HIS | D | 108 | 39.670 | 51.330 | 35.965 | 1.00 | 196.08 |
| 5443 | N | GLY | D | 109 | 37.636 | 52.310 | 35.785 | 1.00 | 171.21 |
| 5444 | CA | GLY | D | 109 | 37.566 | 52.139 | 34.354 | 1.00 | 171.21 |
| 5445 | C | GLY | D | 109 | 38.291 | 53.283 | 33.690 | 1.00 | 171.21 |
| 5446 | O | GLY | D | 109 | 38.608 | 54.272 | 34.344 | 1.00 | 171.21 |
| 5447 | N | TRP | D | 110 | 38.566 | 53.141 | 32.395 | 1.00 | 191.90 |
| 5448 | CA | TRP | D | 110 | 39.258 | 54.181 | 31.637 | 1.00 | 191.90 |
| 5449 | CB | TRP | D | 110 | 40.029 | 53.562 | 30.453 | 1.00 | 203.94 |
| 5450 | CG | TRP | D | 110 | 40.592 | 54.575 | 29.502 | 1.00 | 203.94 |
| 5451 | CD2 | TRP | D | 110 | 41.946 | 55.053 | 29.444 | 1.00 | 203.94 |
| 5452 | CE2 | TRP | D | 110 | 41.995 | 56.035 | 28.434 | 1.00 | 203.94 |
| 5453 | CE3 | TRP | D | 110 | 43.117 | 54.754 | 30.151 | 1.00 | 203.94 |
| 5454 | CD1 | TRP | D | 110 | 39.904 | 55.256 | 28.549 | 1.00 | 203.94 |
| 5455 | NE1 | TRP | D | 110 | 40.740 | 56.136 | 27.907 | 1.00 | 203.94 |
| 5456 | CZ2 | TRP | D | 110 | 43.173 | 56.718 | 28.110 | 1.00 | 203.94 |
| 5457 | CZ3 | TRP | D | 110 | 44.291 | 55.439 | 29.823 | 1.00 | 203.94 |
| 5458 | CH2 | TRP | D | 110 | 44.305 | 56.406 | 28.809 | 1.00 | 203.94 |
| 5459 | C | TRP | D | 110 | 38.245 | 55.224 | 31.155 | 1.00 | 191.90 |
| 5460 | O | TRP | D | 110 | 37.070 | 54.922 | 30.950 | 1.00 | 191.90 |
| 5461 | N | ARG | D | 111 | 38.715 | 56.454 | 30.987 | 1.00 | 162.83 |
| 5462 | CA | ARG | D | 111 | 37.866 | 57.551 | 30.568 | 1.00 | 162.83 |
| 5463 | CB | ARG | D | 111 | 37.487 | 57.418 | 29.098 | 1.00 | 249.69 |
| 5464 | CG | ARG | D | 111 | 38.456 | 58.111 | 28.158 | 1.00 | 249.69 |
| 5465 | CD | ARG | D | 111 | 37.865 | 58.254 | 26.769 | 1.00 | 249.69 |
| 5466 | NE | ARG | D | 111 | 38.203 | 59.544 | 26.175 | 1.00 | 249.69 |
| 5467 | CZ | ARG | D | 111 | 37.865 | 60.717 | 26.705 | 1.00 | 249.69 |
| 5468 | NH1 | ARG | D | 111 | 37.178 | 60.769 | 27.840 | 1.00 | 249.69 |
| 5469 | NH2 | ARG | D | 111 | 38.209 | 61.844 | 26.098 | 1.00 | 249.69 |
| 5470 | C | ARG | D | 111 | 36.612 | 57.597 | 31.415 | 1.00 | 162.83 |
| 5471 | O | ARG | D | 111 | 35.552 | 58.005 | 30.954 | 1.00 | 162.83 |
| 5472 | N | ASN | D | 112 | 36.744 | 57.175 | 32.663 | 1.00 | 190.45 |
| 5473 | CA | ASN | D | 112 | 35.632 | 57.162 | 33.595 | 1.00 | 190.45 |
| 5474 | CB | ASN | D | 112 | 35.305 | 58.593 | 34.044 | 1.00 | 228.54 |
| 5475 | CG | ASN | D | 112 | 34.442 | 58.632 | 35.296 | 1.00 | 228.54 |
| 5476 | OD1 | ASN | D | 112 | 33.860 | 57.622 | 35.701 | 1.00 | 228.54 |
| 5477 | ND2 | ASN | D | 112 | 34.352 | 59.805 | 35.914 | 1.00 | 228.54 |
| 5478 | C | ASN | D | 112 | 34.389 | 56.509 | 32.982 | 1.00 | 190.45 |
| 5479 | O | ASN | D | 112 | 33.263 | 56.879 | 33.318 | 1.00 | 190.45 |
| 5480 | N | TRP | D | 113 | 34.582 | 55.549 | 32.078 | 1.00 | 238.93 |
| 5481 | CA | TRP | D | 113 | 33.437 | 54.868 | 31.475 | 1.00 | 238.93 |
| 5482 | CB | TRP | D | 113 | 33.872 | 53.936 | 30.353 | 1.00 | 249.51 |
| 5483 | CG | TRP | D | 113 | 34.087 | 54.608 | 29.061 | 1.00 | 249.51 |
| 5484 | CD2 | TRP | D | 113 | 35.060 | 54.251 | 28.071 | 1.00 | 249.51 |
| 5485 | CE2 | TRP | D | 113 | 34.860 | 55.110 | 26.973 | 1.00 | 249.51 |
| 5486 | CE3 | TRP | D | 113 | 36.079 | 53.296 | 28.013 | 1.00 | 249.51 |
| 5487 | CD1 | TRP | D | 113 | 33.360 | 55.626 | 28.538 | 1.00 | 249.51 |
| 5488 | NE1 | TRP | D | 113 | 33.817 | 55.940 | 27.283 | 1.00 | 249.51 |
| 5489 | CZ2 | TRP | D | 113 | 35.648 | 55.038 | 25.816 | 1.00 | 249.51 |
| 5490 | CZ3 | TRP | D | 113 | 36.862 | 53.222 | 26.864 | 1.00 | 249.51 |
| 5491 | CH2 | TRP | D | 113 | 36.637 | 54.088 | 25.779 | 1.00 | 249.51 |
| 5492 | C | TRP | D | 113 | 32.733 | 54.037 | 32.530 | 1.00 | 238.93 |
| 5493 | O | TRP | D | 113 | 33.007 | 54.178 | 33.719 | 1.00 | 238.93 |
| 5494 | N | ASP | D | 114 | 31.831 | 53.167 | 32.096 | 1.00 | 249.69 |
| 5495 | CA | ASP | D | 114 | 31.117 | 52.312 | 33.034 | 1.00 | 249.69 |
| 5496 | CB | ASP | D | 114 | 29.600 | 52.451 | 32.843 | 1.00 | 249.69 |
| 5497 | CG | ASP | D | 114 | 29.025 | 53.672 | 33.560 | 1.00 | 249.69 |
| 5498 | OD1 | ASP | D | 114 | 29.189 | 53.780 | 34.798 | 1.00 | 249.69 |
| 5499 | OD2 | ASP | D | 114 | 28.401 | 54.522 | 32.887 | 1.00 | 249.69 |
| 5500 | C | ASP | D | 114 | 31.538 | 50.845 | 32.895 | 1.00 | 249.69 |
| 5501 | O | ASP | D | 114 | 31.612 | 50.312 | 31.778 | 1.00 | 249.69 |
| 5502 | N | VAL | D | 115 | 31.825 | 50.203 | 34.032 | 1.00 | 198.18 |
| 5503 | CA | VAL | D | 115 | 32.232 | 48.800 | 34.043 | 1.00 | 198.18 |
| 5504 | CB | VAL | D | 115 | 33.535 | 48.596 | 34.828 | 1.00 | 157.60 |
| 5505 | CG1 | VAL | D | 115 | 34.102 | 47.220 | 34.521 | 1.00 | 157.60 |
| 5506 | CG2 | VAL | D | 115 | 34.539 | 49.673 | 34.470 | 1.00 | 157.60 |
| 5507 | C | VAL | D | 115 | 31.149 | 47.938 | 34.683 | 1.00 | 198.18 |
| 5508 | O | VAL | D | 115 | 30.530 | 48.329 | 35.681 | 1.00 | 198.18 |
| 5509 | N | TYR | D | 116 | 30.933 | 46.764 | 34.099 | 1.00 | 134.91 |
| 5510 | CA | TYR | D | 116 | 29.922 | 45.824 | 34.578 | 1.00 | 134.91 |
| 5511 | CB | TYR | D | 116 | 28.849 | 45.620 | 33.503 | 1.00 | 249.45 |
| 5512 | CG | TYR | D | 116 | 28.064 | 46.874 | 33.197 | 1.00 | 249.45 |
| 5513 | CD1 | TYR | D | 116 | 28.381 | 47.679 | 32.099 | 1.00 | 249.45 |
| 5514 | CE1 | TYR | D | 116 | 27.673 | 48.861 | 31.844 | 1.00 | 249.45 |
| 5515 | CD2 | TYR | D | 116 | 27.023 | 47.278 | 34.031 | 1.00 | 249.45 |
| 5516 | CE2 | TYR | D | 116 | 26.312 | 48.452 | 33.787 | 1.00 | 249.45 |
| 5517 | CZ | TYR | D | 116 | 26.638 | 49.240 | 32.696 | 1.00 | 249.45 |
| 5518 | OH | TYR | D | 116 | 25.933 | 50.399 | 32.462 | 1.00 | 249.45 |
| 5519 | C | TYR | D | 116 | 30.536 | 44.468 | 34.960 | 1.00 | 134.91 |
| 5520 | O | TYR | D | 116 | 31.706 | 44.205 | 34.670 | 1.00 | 134.91 |
| 5521 | N | LYS | D | 117 | 29.739 | 43.615 | 35.611 | 1.00 | 179.36 |
| 5522 | CA | LYS | D | 117 | 30.185 | 42.289 | 36.054 | 1.00 | 179.36 |
| 5523 | CB | LYS | D | 117 | 30.277 | 41.324 | 34.871 | 1.00 | 249.69 |
| 5524 | CG | LYS | D | 117 | 28.960 | 40.651 | 34.494 | 1.00 | 249.69 |
| 5525 | CD | LYS | D | 117 | 29.202 | 39.387 | 33.661 | 1.00 | 249.69 |
| 5526 | CE | LYS | D | 117 | 30.074 | 38.376 | 34.424 | 1.00 | 249.69 |
| 5527 | NZ | LYS | D | 117 | 30.356 | 37.108 | 33.677 | 1.00 | 249.69 |
| 5528 | C | LYS | D | 117 | 31.531 | 42.338 | 36.769 | 1.00 | 179.36 |
| 5529 | O | LYS | D | 117 | 32.463 | 41.608 | 36.425 | 1.00 | 179.36 |
| 5530 | N | VAL | D | 118 | 31.611 | 43.189 | 37.783 | 1.00 | 143.78 |
| 5531 | CA | VAL | D | 118 | 32.837 | 43.356 | 38.531 | 1.00 | 143.78 |
| 5532 | CB | VAL | D | 118 | 32.928 | 44.783 | 39.076 | 1.00 | 119.69 |
| 5533 | CG1 | VAL | D | 118 | 33.829 | 44.851 | 40.289 | 1.00 | 119.69 |
| 5534 | CG2 | VAL | D | 118 | 33.481 | 45.684 | 37.995 | 1.00 | 119.69 |
| 5535 | C | VAL | D | 118 | 33.071 | 42.366 | 39.664 | 1.00 | 143.78 |
| 5536 | O | VAL | D | 118 | 32.142 | 41.998 | 40.411 | 1.00 | 143.78 |
| 5537 | N | ILE | D | 119 | 34.341 | 41.948 | 39.775 | 1.00 | 115.37 |
| 5538 | CA | ILE | D | 119 | 34.809 | 41.005 | 40.796 | 1.00 | 115.37 |
| 5539 | CB | ILE | D | 119 | 34.981 | 39.618 | 40.220 | 1.00 | 109.20 |
| 5540 | CG2 | ILE | D | 119 | 35.367 | 38.649 | 41.306 | 1.00 | 109.20 |
| 5541 | CG1 | ILE | D | 119 | 33.691 | 39.187 | 39.545 | 1.00 | 109.20 |
| 5542 | CD1 | ILE | D | 119 | 33.917 | 38.153 | 38.481 | 1.00 | 109.20 |
| 5543 | C | ILE | D | 119 | 36.184 | 41.446 | 41.260 | 1.00 | 115.37 |
| 5544 | O | ILE | D | 119 | 37.068 | 41.721 | 40.429 | 1.00 | 115.37 |
| 5545 | N | TYR | D | 120 | 36.364 | 41.538 | 42.573 | 1.00 | 120.82 |
| 5546 | CA | TYR | D | 120 | 37.664 | 41.913 | 43.089 | 1.00 | 120.82 |
| 5547 | CB | TYR | D | 120 | 37.537 | 42.814 | 44.308 | 1.00 | 123.48 |
| 5548 | CG | TYR | D | 120 | 37.016 | 44.181 | 44.008 | 1.00 | 123.48 |
| 5549 | CD1 | TYR | D | 120 | 35.652 | 44.419 | 43.958 | 1.00 | 123.48 |
| 5550 | CE1 | TYR | D | 120 | 35.154 | 45.696 | 43.664 | 1.00 | 123.48 |
| 5551 | CD2 | TYR | D | 120 | 37.889 | 45.246 | 43.758 | 1.00 | 123.48 |
| 5552 | CE2 | TYR | D | 120 | 37.408 | 46.518 | 43.464 | 1.00 | 123.48 |
| 5553 | CZ | TYR | D | 120 | 36.036 | 46.742 | 43.419 | 1.00 | 123.48 |
| 5554 | OH | TYR | D | 120 | 35.552 | 48.010 | 43.132 | 1.00 | 123.48 |
| 5555 | C | TYR | D | 120 | 38.340 | 40.613 | 43.500 | 1.00 | 120.82 |
| 5556 | O | TYR | D | 120 | 37.656 | 39.656 | 43.881 | 1.00 | 120.82 |
| 5557 | N | TYR | D | 121 | 39.672 | 40.567 | 43.420 | 1.00 | 108.96 |
| 5558 | CA | TYR | D | 121 | 40.412 | 39.364 | 43.803 | 1.00 | 108.96 |
| 5559 | CB | TYR | D | 121 | 41.007 | 38.672 | 42.579 | 1.00 | 127.51 |
| 5560 | CG | TYR | D | 121 | 40.034 | 38.043 | 41.600 | 1.00 | 127.51 |
| 5561 | CD1 | TYR | D | 121 | 39.097 | 38.813 | 40.928 | 1.00 | 127.51 |
| 5562 | CE1 | TYR | D | 121 | 38.263 | 38.256 | 39.947 | 1.00 | 127.51 |
| 5563 | CD2 | TYR | D | 121 | 40.116 | 36.690 | 41.277 | 1.00 | 127.51 |
| 5564 | CE2 | TYR | D | 121 | 39.298 | 36.123 | 40.302 | 1.00 | 127.51 |
| 5565 | CZ | TYR | D | 121 | 38.371 | 36.912 | 39.635 | 1.00 | 127.51 |
| 5566 | OH | TYR | D | 121 | 37.566 | 36.374 | 38.642 | 1.00 | 127.51 |
| 5567 | C | TYR | D | 121 | 41.557 | 39.682 | 44.755 | 1.00 | 108.96 |
| 5568 | O | TYR | D | 121 | 42.328 | 40.624 | 44.539 | 1.00 | 108.96 |
| 5569 | N | LYS | D | 122 | 41.666 | 38.885 | 45.807 | 1.00 | 150.86 |
| 5570 | CA | LYS | D | 122 | 42.741 | 39.060 | 46.762 | 1.00 | 150.86 |
| 5571 | CB | LYS | D | 122 | 42.199 | 39.419 | 48.145 | 1.00 | 185.53 |
| 5572 | CG | LYS | D | 122 | 43.292 | 39.629 | 49.176 | 1.00 | 185.53 |
| 5573 | CD | LYS | D | 122 | 42.724 | 39.656 | 50.576 | 1.00 | 185.53 |
| 5574 | CE | LYS | D | 122 | 43.826 | 39.686 | 51.615 | 1.00 | 185.53 |
| 5575 | NZ | LYS | D | 122 | 43.245 | 39.553 | 52.970 | 1.00 | 185.53 |
| 5576 | C | LYS | D | 122 | 43.496 | 37.738 | 46.834 | 1.00 | 150.86 |
| 5577 | O | LYS | D | 122 | 42.928 | 36.707 | 47.210 | 1.00 | 150.86 |
| 5578 | N | ASP | D | 123 | 44.771 | 37.771 | 46.463 | 1.00 | 129.20 |
| 5579 | CA | ASP | D | 123 | 45.601 | 36.577 | 46.485 | 1.00 | 129.20 |
| 5580 | CB | ASP | D | 123 | 45.857 | 36.104 | 47.924 | 1.00 | 160.45 |
| 5581 | CG | ASP | D | 123 | 46.852 | 36.986 | 48.661 | 1.00 | 160.45 |
| 5582 | OD1 | ASP | D | 123 | 47.927 | 37.268 | 48.096 | 1.00 | 160.45 |
| 5583 | OD2 | ASP | D | 123 | 46.572 | 37.390 | 49.808 | 1.00 | 160.45 |
| 5584 | C | ASP | D | 123 | 44.999 | 35.441 | 45.687 | 1.00 | 129.20 |
| 5585 | O | ASP | D | 123 | 44.855 | 34.331 | 46.205 | 1.00 | 129.20 |
| 5586 | N | GLY | D | 124 | 44.643 | 35.729 | 44.434 | 1.00 | 131.93 |
| 5587 | CA | GLY | D | 124 | 44.085 | 34.715 | 43.547 | 1.00 | 131.93 |
| 5588 | C | GLY | D | 124 | 42.682 | 34.206 | 43.837 | 1.00 | 131.93 |
| 5589 | O | GLY | D | 124 | 42.137 | 33.416 | 43.058 | 1.00 | 131.93 |
| 5590 | N | GLU | D | 125 | 42.093 | 34.656 | 44.942 | 1.00 | 141.53 |
| 5591 | CA | GLU | D | 125 | 40.751 | 34.229 | 45.321 | 1.00 | 141.53 |
| 5592 | CB | GLU | D | 125 | 40.682 | 34.033 | 46.840 | 1.00 | 249.69 |
| 5593 | CG | GLU | D | 125 | 41.469 | 32.842 | 47.372 | 1.00 | 249.69 |
| 5594 | CD | GLU | D | 125 | 40.788 | 31.512 | 47.074 | 1.00 | 249.69 |
| 5595 | OE1 | GLU | D | 125 | 39.664 | 31.297 | 47.571 | 1.00 | 249.69 |
| 5596 | OE2 | GLU | D | 125 | 41.373 | 30.682 | 46.346 | 1.00 | 249.69 |
| 5597 | C | GLU | D | 125 | 39.673 | 35.224 | 44.897 | 1.00 | 141.53 |
| 5598 | O | GLU | D | 125 | 39.903 | 36.441 | 44.907 | 1.00 | 141.53 |
| 5599 | N | ALA | D | 126 | 38.503 | 34.707 | 44.520 | 1.00 | 148.59 |
| 5600 | CA | ALA | D | 126 | 37.393 | 35.570 | 44.136 | 1.00 | 148.59 |
| 5601 | CB | ALA | D | 126 | 36.274 | 34.743 | 43.560 | 1.00 | 144.26 |
| 5602 | C | ALA | D | 126 | 36.961 | 36.205 | 45.453 | 1.00 | 148.59 |
| 5603 | O | ALA | D | 126 | 36.909 | 35.516 | 46.481 | 1.00 | 148.59 |
| 5604 | N | LEU | D | 127 | 36.652 | 37.501 | 45.448 | 1.00 | 169.46 |
| 5605 | CA | LEU | D | 127 | 36.274 | 38.153 | 46.700 | 1.00 | 169.46 |
| 5606 | CB | LEU | D | 127 | 37.294 | 39.224 | 47.040 | 1.00 | 146.34 |
| 5607 | CG | LEU | D | 127 | 37.368 | 39.389 | 48.547 | 1.00 | 146.34 |
| 5608 | CD1 | LEU | D | 127 | 37.671 | 38.030 | 49.183 | 1.00 | 146.34 |
| 5609 | CD2 | LEU | D | 127 | 38.440 | 40.394 | 48.897 | 1.00 | 146.34 |
| 5610 | C | LEU | D | 127 | 34.880 | 38.750 | 46.796 | 1.00 | 169.46 |
| 5611 | O | LEU | D | 127 | 34.081 | 38.350 | 47.640 | 1.00 | 169.46 |
| 5612 | N | LYS | D | 128 | 34.609 | 39.738 | 45.957 | 1.00 | 129.34 |
| 5613 | CA | LYS | D | 128 | 33.302 | 40.371 | 45.930 | 1.00 | 129.34 |
| 5614 | CB | LYS | D | 128 | 33.390 | 41.784 | 46.505 | 1.00 | 216.92 |
| 5615 | CG | LYS | D | 128 | 33.863 | 41.849 | 47.952 | 1.00 | 216.92 |
| 5616 | CD | LYS | D | 128 | 32.806 | 41.345 | 48.935 | 1.00 | 216.92 |
| 5617 | CE | LYS | D | 128 | 33.279 | 41.519 | 50.376 | 1.00 | 216.92 |
| 5618 | NZ | LYS | D | 128 | 32.194 | 41.270 | 51.366 | 1.00 | 216.92 |
| 5619 | C | LYS | D | 128 | 32.834 | 40.419 | 44.475 | 1.00 | 129.34 |
| 5620 | O | LYS | D | 128 | 33.645 | 40.314 | 43.556 | 1.00 | 129.34 |
| 5621 | N | TYR | D | 129 | 31.532 | 40.581 | 44.261 | 1.00 | 159.52 |
| 5622 | CA | TYR | D | 129 | 31.000 | 40.642 | 42.907 | 1.00 | 159.52 |
| 5623 | CB | TYR | D | 129 | 30.682 | 39.239 | 42.432 | 1.00 | 146.13 |
| 5624 | CG | TYR | D | 129 | 29.763 | 39.234 | 41.246 | 1.00 | 146.13 |
| 5625 | CD1 | TYR | D | 129 | 30.255 | 39.420 | 39.958 | 1.00 | 146.13 |
| 5626 | CE1 | TYR | D | 129 | 29.395 | 39.478 | 38.859 | 1.00 | 146.13 |
| 5627 | CD2 | TYR | D | 129 | 28.380 | 39.103 | 41.419 | 1.00 | 146.13 |
| 5628 | CE2 | TYR | D | 129 | 27.507 | 39.162 | 40.337 | 1.00 | 146.13 |
| 5629 | CZ | TYR | D | 129 | 28.021 | 39.350 | 39.055 | 1.00 | 146.13 |
| 5630 | OH | TYR | D | 129 | 27.158 | 39.406 | 37.976 | 1.00 | 146.13 |
| 5631 | C | TYR | D | 129 | 29.747 | 41.516 | 42.767 | 1.00 | 159.52 |
| 5632 | O | TYR | D | 129 | 28.858 | 41.489 | 43.622 | 1.00 | 159.52 |
| 5633 | N | TRP | D | 130 | 29.676 | 42.283 | 41.678 | 1.00 | 181.39 |
| 5634 | CA | TRP | D | 130 | 28.519 | 43.144 | 41.418 | 1.00 | 181.39 |
| 5635 | CB | TRP | D | 130 | 28.703 | 44.540 | 42.021 | 1.00 | 248.73 |
| 5636 | CG | TRP | D | 130 | 29.193 | 44.604 | 43.436 | 1.00 | 248.73 |
| 5637 | CD2 | TRP | D | 130 | 28.426 | 44.935 | 44.598 | 1.00 | 248.73 |
| 5638 | CE2 | TRP | D | 130 | 29.302 | 44.930 | 45.703 | 1.00 | 248.73 |
| 5639 | CE3 | TRP | D | 130 | 27.074 | 45.246 | 44.816 | 1.00 | 248.73 |
| 5640 | CD1 | TRP | D | 130 | 30.471 | 44.405 | 43.872 | 1.00 | 248.73 |
| 5641 | NE1 | TRP | D | 130 | 30.551 | 44.606 | 45.229 | 1.00 | 248.73 |
| 5642 | CZ2 | TRP | D | 130 | 28.883 | 45.219 | 47.005 | 1.00 | 248.73 |
| 5643 | CZ3 | TRP | D | 130 | 26.651 | 45.532 | 46.116 | 1.00 | 248.73 |
| 5644 | CH2 | TRP | D | 130 | 27.555 | 45.511 | 47.192 | 1.00 | 248.73 |
| 5645 | C | TRP | D | 130 | 28.281 | 43.326 | 39.916 | 1.00 | 181.39 |
| 5646 | O | TRP | D | 130 | 29.126 | 42.952 | 39.090 | 1.00 | 181.39 |
| 5647 | N | TYR | D | 131 | 27.129 | 43.907 | 39.576 | 1.00 | 195.20 |
| 5648 | CA | TYR | D | 131 | 26.776 | 44.183 | 38.185 | 1.00 | 195.20 |
| 5649 | CB | TYR | D | 131 | 25.263 | 44.162 | 38.020 | 1.00 | 249.67 |
| 5650 | CG | TYR | D | 131 | 24.831 | 44.143 | 36.579 | 1.00 | 249.67 |
| 5651 | CD1 | TYR | D | 131 | 24.974 | 42.992 | 35.806 | 1.00 | 249.67 |
| 5652 | CE1 | TYR | D | 131 | 24.612 | 42.979 | 34.465 | 1.00 | 249.67 |
| 5653 | CD2 | TYR | D | 131 | 24.311 | 45.285 | 35.973 | 1.00 | 249.67 |
| 5654 | CE2 | TYR | D | 131 | 23.949 | 45.285 | 34.632 | 1.00 | 249.67 |
| 5655 | CZ | TYR | D | 131 | 24.101 | 44.131 | 33.884 | 1.00 | 249.67 |
| 5656 | OH | TYR | D | 131 | 23.751 | 44.140 | 32.553 | 1.00 | 249.67 |
| 5657 | C | TYR | D | 131 | 27.319 | 45.591 | 37.904 | 1.00 | 195.20 |
| 5658 | O | TYR | D | 131 | 28.458 | 45.746 | 37.468 | 1.00 | 195.20 |
| 5659 | N | GLU | D | 132 | 26.492 | 46.614 | 38.131 | 1.00 | 246.45 |
| 5660 | CA | GLU | D | 132 | 26.949 | 47.994 | 37.982 | 1.00 | 246.45 |
| 5661 | CB | GLU | D | 132 | 25.841 | 48.983 | 38.357 | 1.00 | 249.69 |
| 5662 | CG | GLU | D | 132 | 24.774 | 49.219 | 37.292 | 1.00 | 249.69 |
| 5663 | CD | GLU | D | 132 | 24.762 | 50.658 | 36.806 | 1.00 | 249.69 |
| 5664 | OE1 | GLU | D | 132 | 25.408 | 51.508 | 37.461 | 1.00 | 249.69 |
| 5665 | OE2 | GLU | D | 132 | 24.106 | 50.945 | 35.777 | 1.00 | 249.69 |
| 5666 | C | GLU | D | 132 | 27.976 | 47.929 | 39.090 | 1.00 | 246.45 |
| 5667 | O | GLU | D | 132 | 27.639 | 47.527 | 40.210 | 1.00 | 246.45 |
| 5668 | N | ASN | D | 133 | 29.219 | 48.320 | 38.821 | 1.00 | 125.13 |
| 5669 | CA | ASN | D | 133 | 30.220 | 48.146 | 39.877 | 1.00 | 125.13 |
| 5670 | CB | ASN | D | 133 | 31.670 | 48.261 | 39.299 | 1.00 | 124.76 |
| 5671 | CG | ASN | D | 133 | 32.189 | 49.671 | 39.168 | 1.00 | 124.76 |
| 5672 | OD1 | ASN | D | 133 | 31.488 | 50.569 | 38.725 | 1.00 | 124.76 |
| 5673 | ND2 | ASN | D | 133 | 33.462 | 49.855 | 39.512 | 1.00 | 124.76 |
| 5674 | C | ASN | D | 133 | 30.069 | 48.859 | 41.223 | 1.00 | 125.13 |
| 5675 | O | ASN | D | 133 | 29.046 | 49.479 | 41.527 | 1.00 | 125.13 |
| 5676 | N | HIS | D | 134 | 31.077 | 48.688 | 42.057 | 1.00 | 175.64 |
| 5677 | CA | HIS | D | 134 | 31.054 | 49.259 | 43.375 | 1.00 | 175.64 |
| 5678 | CB | HIS | D | 134 | 30.511 | 48.218 | 44.358 | 1.00 | 249.69 |
| 5679 | CG | HIS | D | 134 | 30.264 | 48.759 | 45.738 | 1.00 | 249.69 |
| 5680 | CD2 | HIS | D | 134 | 30.834 | 48.440 | 46.925 | 1.00 | 249.69 |
| 5681 | ND1 | HIS | D | 134 | 29.361 | 49.759 | 45.988 | 1.00 | 249.69 |
| 5682 | CE1 | HIS | D | 134 | 29.377 | 50.048 | 47.287 | 1.00 | 249.69 |
| 5683 | NE2 | HIS | D | 134 | 30.260 | 49.263 | 47.870 | 1.00 | 249.69 |
| 5684 | C | HIS | D | 134 | 32.481 | 49.650 | 43.733 | 1.00 | 175.64 |
| 5685 | O | HIS | D | 134 | 33.352 | 49.738 | 42.862 | 1.00 | 175.64 |
| 5686 | N | ASN | D | 135 | 32.714 | 49.878 | 45.020 | 1.00 | 171.27 |
| 5687 | CA | ASN | D | 135 | 34.020 | 50.269 | 45.510 | 1.00 | 171.27 |
| 5688 | CB | ASN | D | 135 | 34.116 | 51.799 | 45.567 | 1.00 | 249.69 |
| 5689 | CG | ASN | D | 135 | 34.113 | 52.439 | 44.180 | 1.00 | 249.69 |
| 5690 | OD1 | ASN | D | 135 | 34.830 | 51.972 | 43.295 | 1.00 | 249.69 |
| 5691 | ND2 | ASN | D | 135 | 33.336 | 53.512 | 43.992 | 1.00 | 249.69 |
| 5692 | C | ASN | D | 135 | 34.237 | 49.660 | 46.894 | 1.00 | 171.27 |
| 5693 | O | ASN | D | 135 | 34.009 | 50.303 | 47.907 | 1.00 | 171.27 |
| 5694 | N | ILE | D | 136 | 34.670 | 48.405 | 46.916 | 1.00 | 141.21 |
| 5695 | CA | ILE | D | 136 | 34.953 | 47.636 | 48.143 | 1.00 | 141.21 |
| 5696 | CB | ILE | D | 136 | 35.894 | 46.432 | 47.813 | 1.00 | 122.14 |
| 5697 | CG2 | ILE | D | 136 | 37.169 | 46.918 | 47.141 | 1.00 | 122.14 |
| 5698 | CG1 | ILE | D | 136 | 36.246 | 45.665 | 49.068 | 1.00 | 122.14 |
| 5699 | CD1 | ILE | D | 136 | 37.202 | 44.530 | 48.789 | 1.00 | 122.14 |
| 5700 | C | ILE | D | 136 | 35.571 | 48.458 | 49.276 | 1.00 | 141.21 |
| 5701 | O | ILE | D | 136 | 36.769 | 48.809 | 49.257 | 1.00 | 141.21 |
| 5702 | N | SER | D | 137 | 34.751 | 48.728 | 50.282 | 1.00 | 155.34 |
| 5703 | CA | SER | D | 137 | 35.189 | 49.539 | 51.415 | 1.00 | 155.34 |
| 5704 | CB | SER | D | 137 | 34.179 | 50.648 | 51.662 | 1.00 | 178.90 |
| 5705 | OG | SER | D | 137 | 34.452 | 51.311 | 52.884 | 1.00 | 178.90 |
| 5706 | C | SER | D | 137 | 35.447 | 48.817 | 52.739 | 1.00 | 155.34 |
| 5707 | O | SER | D | 137 | 34.804 | 47.815 | 53.067 | 1.00 | 155.34 |
| 5708 | N | ILE | D | 138 | 36.378 | 49.372 | 53.509 | 1.00 | 165.15 |
| 5709 | CA | ILE | D | 138 | 36.770 | 48.823 | 54.792 | 1.00 | 165.15 |
| 5710 | CB | ILE | D | 138 | 38.095 | 48.075 | 54.654 | 1.00 | 128.87 |
| 5711 | CG2 | ILE | D | 138 | 38.690 | 47.804 | 56.022 | 1.00 | 128.87 |
| 5712 | CG1 | ILE | D | 138 | 37.861 | 46.780 | 53.894 | 1.00 | 128.87 |
| 5713 | CD1 | ILE | D | 138 | 39.131 | 46.098 | 53.483 | 1.00 | 128.87 |
| 5714 | C | ILE | D | 138 | 36.919 | 49.901 | 55.863 | 1.00 | 165.15 |
| 5715 | O | ILE | D | 138 | 37.703 | 50.849 | 55.720 | 1.00 | 165.15 |
| 5716 | N | THR | D | 139 | 36.167 | 49.733 | 56.944 | 1.00 | 191.18 |
| 5717 | CA | THR | D | 139 | 36.186 | 50.663 | 58.065 | 1.00 | 191.18 |
| 5718 | CB | THR | D | 139 | 34.891 | 50.533 | 58.855 | 1.00 | 246.32 |
| 5719 | OG1 | THR | D | 139 | 34.694 | 49.160 | 59.210 | 1.00 | 246.32 |
| 5720 | CG2 | THR | D | 139 | 33.713 | 50.988 | 58.006 | 1.00 | 246.32 |
| 5721 | C | THR | D | 139 | 37.364 | 50.342 | 58.976 | 1.00 | 191.18 |
| 5722 | O | THR | D | 139 | 38.413 | 50.981 | 58.911 | 1.00 | 191.18 |
| 5723 | N | ASN | D | 140 | 37.173 | 49.343 | 59.827 | 1.00 | 193.50 |
| 5724 | CA | ASN | D | 140 | 38.211 | 48.887 | 60.742 | 1.00 | 193.50 |
| 5725 | CB | ASN | D | 140 | 37.561 | 48.246 | 61.967 | 1.00 | 183.46 |
| 5726 | CG | ASN | D | 140 | 38.567 | 47.701 | 62.943 | 1.00 | 183.46 |
| 5727 | OD1 | ASN | D | 140 | 39.474 | 46.972 | 62.554 | 1.00 | 183.46 |
| 5728 | ND2 | ASN | D | 140 | 38.403 | 48.038 | 64.218 | 1.00 | 183.46 |
| 5729 | C | ASN | D | 140 | 39.022 | 47.849 | 59.960 | 1.00 | 193.50 |
| 5730 | O | ASN | D | 140 | 38.472 | 46.846 | 59.482 | 1.00 | 193.50 |
| 5731 | N | ALA | D | 141 | 40.324 | 48.088 | 59.822 | 1.00 | 163.85 |
| 5732 | CA | ALA | D | 141 | 41.190 | 47.179 | 59.061 | 1.00 | 163.85 |
| 5733 | CB | ALA | D | 141 | 42.181 | 48.002 | 58.187 | 1.00 | 57.61 |
| 5734 | C | ALA | D | 141 | 41.956 | 46.131 | 59.872 | 1.00 | 163.85 |
| 5735 | O | ALA | D | 141 | 42.669 | 46.446 | 60.823 | 1.00 | 163.85 |
| 5736 | N | THR | D | 142 | 41.794 | 44.880 | 59.470 | 1.00 | 158.30 |
| 5737 | CA | THR | D | 142 | 42.464 | 43.765 | 60.113 | 1.00 | 158.30 |
| 5738 | CB | THR | D | 142 | 41.654 | 42.471 | 59.957 | 1.00 | 191.01 |
| 5739 | OG1 | THR | D | 142 | 40.299 | 42.703 | 60.352 | 1.00 | 191.01 |
| 5740 | CG2 | THR | D | 142 | 42.248 | 41.370 | 60.813 | 1.00 | 191.01 |
| 5741 | C | THR | D | 142 | 43.798 | 43.568 | 59.407 | 1.00 | 158.30 |
| 5742 | O | THR | D | 142 | 43.992 | 44.042 | 58.282 | 1.00 | 158.30 |
| 5743 | N | VAL | D | 143 | 44.723 | 42.867 | 60.052 | 1.00 | 168.28 |
| 5744 | CA | VAL | D | 143 | 46.017 | 42.637 | 59.430 | 1.00 | 168.28 |
| 5745 | CB | VAL | D | 143 | 47.063 | 42.169 | 60.441 | 1.00 | 249.69 |
| 5746 | CG1 | VAL | D | 143 | 46.777 | 40.734 | 60.851 | 1.00 | 249.69 |
| 5747 | CG2 | VAL | D | 143 | 48.453 | 42.298 | 59.830 | 1.00 | 249.69 |
| 5748 | C | VAL | D | 143 | 45.893 | 41.580 | 58.357 | 1.00 | 168.28 |
| 5749 | O | VAL | D | 143 | 46.711 | 41.521 | 57.446 | 1.00 | 168.28 |
| 5750 | N | GLU | D | 144 | 44.874 | 40.737 | 58.469 | 1.00 | 197.52 |
| 5751 | CA | GLU | D | 144 | 44.671 | 39.694 | 57.475 | 1.00 | 197.52 |
| 5752 | CB | GLU | D | 144 | 43.667 | 38.654 | 57.965 | 1.00 | 249.69 |
| 5753 | CG | GLU | D | 144 | 44.088 | 37.957 | 59.232 | 1.00 | 249.69 |
| 5754 | CD | GLU | D | 144 | 43.210 | 38.332 | 60.397 | 1.00 | 249.69 |
| 5755 | OE1 | GLU | D | 144 | 41.994 | 38.051 | 60.329 | 1.00 | 249.69 |
| 5756 | OE2 | GLU | D | 144 | 43.729 | 38.910 | 61.376 | 1.00 | 249.69 |
| 5757 | C | GLU | D | 144 | 44.186 | 40.286 | 56.154 | 1.00 | 197.52 |
| 5758 | O | GLU | D | 144 | 44.159 | 39.591 | 55.137 | 1.00 | 197.52 |
| 5759 | N | ASP | D | 145 | 43.805 | 41.565 | 56.173 | 1.00 | 135.76 |
| 5760 | CA | ASP | D | 145 | 43.346 | 42.243 | 54.965 | 1.00 | 135.76 |
| 5761 | CB | ASP | D | 145 | 42.617 | 43.538 | 55.311 | 1.00 | 217.88 |
| 5762 | CG | ASP | D | 145 | 41.206 | 43.293 | 55.813 | 1.00 | 217.88 |
| 5763 | OD1 | ASP | D | 145 | 40.415 | 42.658 | 55.081 | 1.00 | 217.88 |
| 5764 | OD2 | ASP | D | 145 | 40.881 | 43.737 | 56.938 | 1.00 | 217.88 |
| 5765 | C | ASP | D | 145 | 44.512 | 42.549 | 54.030 | 1.00 | 135.76 |
| 5766 | O | ASP | D | 145 | 44.319 | 42.840 | 52.851 | 1.00 | 135.76 |
| 5767 | N | SER | D | 146 | 45.728 | 42.478 | 54.559 | 1.00 | 129.49 |
| 5768 | CA | SER | D | 146 | 46.945 | 42.736 | 53.778 | 1.00 | 129.49 |
| 5769 | CB | SER | D | 146 | 48.185 | 42.741 | 54.696 | 1.00 | 138.30 |
| 5770 | OG | SER | D | 146 | 48.092 | 43.709 | 55.730 | 1.00 | 138.30 |
| 5771 | C | SER | D | 146 | 47.128 | 41.662 | 52.709 | 1.00 | 129.49 |
| 5772 | O | SER | D | 146 | 47.094 | 40.471 | 53.005 | 1.00 | 129.49 |
| 5773 | N | GLY | D | 147 | 47.335 | 42.079 | 51.466 | 1.00 | 156.91 |
| 5774 | CA | GLY | D | 147 | 47.534 | 41.109 | 50.400 | 1.00 | 156.91 |
| 5775 | C | GLY | D | 147 | 47.729 | 41.750 | 49.041 | 1.00 | 156.91 |
| 5776 | O | GLY | D | 147 | 48.071 | 42.922 | 48.948 | 1.00 | 156.91 |
| 5777 | N | THR | D | 148 | 47.514 | 40.983 | 47.980 | 1.00 | 120.73 |
| 5778 | CA | THR | D | 148 | 47.663 | 41.506 | 46.626 | 1.00 | 120.73 |
| 5779 | CB | THR | D | 148 | 48.770 | 40.742 | 45.861 | 1.00 | 132.18 |
| 5780 | OG1 | THR | D | 148 | 48.194 | 39.716 | 45.043 | 1.00 | 132.18 |
| 5781 | CG2 | THR | D | 148 | 49.728 | 40.096 | 46.837 | 1.00 | 132.18 |
| 5782 | C | THR | D | 148 | 46.320 | 41.438 | 45.848 | 1.00 | 120.73 |
| 5783 | O | THR | D | 148 | 45.808 | 40.338 | 45.526 | 1.00 | 120.73 |
| 5784 | N | TYR | D | 149 | 45.757 | 42.618 | 45.545 | 1.00 | 89.32 |
| 5785 | CA | TYR | D | 149 | 44.471 | 42.706 | 44.849 | 1.00 | 89.32 |
| 5786 | CB | TYR | D | 149 | 43.573 | 43.748 | 45.540 | 1.00 | 105.54 |
| 5787 | CG | TYR | D | 149 | 43.303 | 43.551 | 47.020 | 1.00 | 105.54 |
| 5788 | CD1 | TYR | D | 149 | 44.281 | 43.842 | 47.984 | 1.00 | 105.54 |
| 5789 | CE1 | TYR | D | 149 | 44.016 | 43.690 | 49.350 | 1.00 | 105.54 |
| 5790 | CD2 | TYR | D | 149 | 42.056 | 43.098 | 47.460 | 1.00 | 105.54 |
| 5791 | CE2 | TYR | D | 149 | 41.781 | 42.942 | 48.812 | 1.00 | 105.54 |
| 5792 | CZ | TYR | D | 149 | 42.761 | 43.237 | 49.747 | 1.00 | 105.54 |
| 5793 | OH | TYR | D | 149 | 42.470 | 43.085 | 51.077 | 1.00 | 105.54 |
| 5794 | C | TYR | D | 149 | 44.565 | 43.068 | 43.360 | 1.00 | 89.32 |
| 5795 | O | TYR | D | 149 | 45.586 | 43.579 | 42.877 | 1.00 | 89.32 |
| 5796 | N | TYR | D | 150 | 43.462 | 42.806 | 42.662 | 1.00 | 127.86 |
| 5797 | CA | TYR | D | 150 | 43.278 | 43.117 | 41.246 | 1.00 | 127.86 |
| 5798 | CB | TYR | D | 150 | 44.146 | 42.218 | 40.355 | 1.00 | 148.19 |
| 5799 | CG | TYR | D | 150 | 43.643 | 40.801 | 40.109 | 1.00 | 148.19 |
| 5800 | CD1 | TYR | D | 150 | 42.539 | 40.554 | 39.296 | 1.00 | 148.19 |
| 5801 | CE1 | TYR | D | 150 | 42.105 | 39.243 | 39.025 | 1.00 | 148.19 |
| 5802 | CD2 | TYR | D | 150 | 44.308 | 39.701 | 40.649 | 1.00 | 148.19 |
| 5803 | CE2 | TYR | D | 150 | 43.888 | 38.381 | 40.378 | 1.00 | 148.19 |
| 5804 | CZ | TYR | D | 150 | 42.785 | 38.163 | 39.563 | 1.00 | 148.19 |
| 5805 | OH | TYR | D | 150 | 42.376 | 36.876 | 39.273 | 1.00 | 148.19 |
| 5806 | C | TYR | D | 150 | 41.790 | 42.882 | 40.976 | 1.00 | 127.86 |
| 5807 | O | TYR | D | 150 | 41.157 | 42.091 | 41.681 | 1.00 | 127.86 |
| 5808 | N | CYS | D | 151 | 41.218 | 43.567 | 39.987 | 1.00 | 122.94 |
| 5809 | CA | CYS | D | 151 | 39.793 | 43.385 | 39.685 | 1.00 | 122.94 |
| 5810 | C | CYS | D | 151 | 39.559 | 43.047 | 38.224 | 1.00 | 122.94 |
| 5811 | O | CYS | D | 151 | 40.438 | 43.275 | 37.379 | 1.00 | 122.94 |
| 5812 | CB | CYS | D | 151 | 39.010 | 44.640 | 40.049 | 1.00 | 183.39 |
| 5813 | SG | CYS | D | 151 | 39.522 | 46.144 | 39.169 | 1.00 | 183.39 |
| 5814 | N | THR | D | 152 | 38.379 | 42.494 | 37.935 | 1.00 | 139.21 |
| 5815 | CA | THR | D | 152 | 38.013 | 42.119 | 36.565 | 1.00 | 139.21 |
| 5816 | CB | THR | D | 152 | 37.955 | 40.598 | 36.383 | 1.00 | 172.57 |
| 5817 | OG1 | THR | D | 152 | 36.776 | 40.090 | 37.025 | 1.00 | 172.57 |
| 5818 | CG2 | THR | D | 152 | 39.185 | 39.945 | 36.985 | 1.00 | 172.57 |
| 5819 | C | THR | D | 152 | 36.627 | 42.660 | 36.247 | 1.00 | 139.21 |
| 5820 | O | THR | D | 152 | 35.765 | 42.721 | 37.124 | 1.00 | 139.21 |
| 5821 | N | GLY | D | 153 | 36.411 | 43.037 | 34.993 | 1.00 | 182.81 |
| 5822 | CA | GLY | D | 153 | 35.115 | 43.562 | 34.620 | 1.00 | 182.81 |
| 5823 | C | GLY | D | 153 | 34.905 | 43.665 | 33.126 | 1.00 | 182.81 |
| 5824 | O | GLY | D | 153 | 35.844 | 43.520 | 32.350 | 1.00 | 182.81 |
| 5825 | N | LYS | D | 154 | 33.662 | 43.917 | 32.730 | 1.00 | 140.36 |
| 5826 | CA | LYS | D | 154 | 33.307 | 44.047 | 31.327 | 1.00 | 140.36 |
| 5827 | CB | LYS | D | 154 | 32.064 | 43.211 | 31.040 | 1.00 | 249.69 |
| 5828 | CG | LYS | D | 154 | 31.649 | 43.177 | 29.581 | 1.00 | 249.69 |
| 5829 | CD | LYS | D | 154 | 30.442 | 42.266 | 29.384 | 1.00 | 249.69 |
| 5830 | CE | LYS | D | 154 | 29.973 | 42.264 | 27.938 | 1.00 | 249.69 |
| 5831 | NZ | LYS | D | 154 | 28.786 | 41.385 | 27.740 | 1.00 | 249.69 |
| 5832 | C | LYS | D | 154 | 33.055 | 45.519 | 30.936 | 1.00 | 140.36 |
| 5833 | O | LYS | D | 154 | 32.150 | 46.184 | 31.458 | 1.00 | 140.36 |
| 5834 | N | VAL | D | 155 | 33.881 | 46.020 | 30.021 | 1.00 | 200.49 |
| 5835 | CA | VAL | D | 155 | 33.793 | 47.390 | 29.517 | 1.00 | 200.49 |
| 5836 | CB | VAL | D | 155 | 35.198 | 48.038 | 29.434 | 1.00 | 172.58 |
| 5837 | CG1 | VAL | D | 155 | 35.116 | 49.415 | 28.834 | 1.00 | 172.58 |
| 5838 | CG2 | VAL | D | 155 | 35.811 | 48.111 | 30.820 | 1.00 | 172.58 |
| 5839 | C | VAL | D | 155 | 33.211 | 47.276 | 28.116 | 1.00 | 200.49 |
| 5840 | O | VAL | D | 155 | 33.711 | 46.504 | 27.298 | 1.00 | 200.49 |
| 5841 | N | TRP | D | 156 | 32.169 | 48.051 | 27.831 | 1.00 | 193.00 |
| 5842 | CA | TRP | D | 156 | 31.502 | 47.986 | 26.522 | 1.00 | 193.00 |
| 5843 | CB | TRP | D | 156 | 32.472 | 48.222 | 25.344 | 1.00 | 249.69 |
| 5844 | CG | TRP | D | 156 | 33.061 | 49.610 | 25.206 | 1.00 | 249.69 |
| 5845 | CD2 | TRP | D | 156 | 32.372 | 50.817 | 24.849 | 1.00 | 249.69 |
| 5846 | CE2 | TRP | D | 156 | 33.330 | 51.857 | 24.815 | 1.00 | 249.69 |
| 5847 | CE3 | TRP | D | 156 | 31.047 | 51.121 | 24.550 | 1.00 | 249.69 |
| 5848 | CD1 | TRP | D | 156 | 34.372 | 49.961 | 25.377 | 1.00 | 249.69 |
| 5849 | NE1 | TRP | D | 156 | 34.537 | 51.309 | 25.141 | 1.00 | 249.69 |
| 5850 | CZ2 | TRP | D | 156 | 32.996 | 53.172 | 24.499 | 1.00 | 249.69 |
| 5851 | CZ3 | TRP | D | 156 | 30.729 | 52.433 | 24.235 | 1.00 | 249.69 |
| 5852 | CH2 | TRP | D | 156 | 31.692 | 53.438 | 24.219 | 1.00 | 249.69 |
| 5853 | C | TRP | D | 156 | 30.980 | 46.564 | 26.418 | 1.00 | 193.00 |
| 5854 | O | TRP | D | 156 | 29.921 | 46.232 | 26.957 | 1.00 | 193.00 |
| 5855 | N | GLN | D | 157 | 31.755 | 45.727 | 25.732 | 1.00 | 206.01 |
| 5856 | CA | GLN | D | 157 | 31.402 | 44.330 | 25.555 | 1.00 | 206.01 |
| 5857 | CB | GLN | D | 157 | 30.644 | 44.150 | 24.236 | 1.00 | 249.69 |
| 5858 | CG | GLN | D | 157 | 29.201 | 44.650 | 24.285 | 1.00 | 249.69 |
| 5859 | CD | GLN | D | 157 | 28.329 | 43.857 | 25.262 | 1.00 | 249.69 |
| 5860 | OE1 | GLN | D | 157 | 28.115 | 42.651 | 25.090 | 1.00 | 249.69 |
| 5861 | NE2 | GLN | D | 157 | 27.820 | 44.535 | 26.291 | 1.00 | 249.69 |
| 5862 | C | GLN | D | 157 | 32.585 | 43.353 | 25.631 | 1.00 | 206.01 |
| 5863 | O | GLN | D | 157 | 32.508 | 42.233 | 25.122 | 1.00 | 206.01 |
| 5864 | N | LEU | D | 158 | 33.674 | 43.773 | 26.269 | 1.00 | 203.26 |
| 5865 | CA | LEU | D | 158 | 34.833 | 42.900 | 26.427 | 1.00 | 203.26 |
| 5866 | CB | LEU | D | 158 | 35.991 | 43.350 | 25.529 | 1.00 | 242.89 |
| 5867 | CG | LEU | D | 158 | 35.914 | 43.035 | 24.033 | 1.00 | 242.89 |
| 5868 | CD1 | LEU | D | 158 | 37.324 | 42.735 | 23.537 | 1.00 | 242.89 |
| 5869 | CD2 | LEU | D | 158 | 35.015 | 41.829 | 23.772 | 1.00 | 242.89 |
| 5870 | C | LEU | D | 158 | 35.301 | 42.846 | 27.878 | 1.00 | 203.26 |
| 5871 | O | LEU | D | 158 | 35.127 | 43.802 | 28.629 | 1.00 | 203.26 |
| 5872 | N | ASP | D | 159 | 35.886 | 41.718 | 28.268 | 1.00 | 176.99 |
| 5873 | CA | ASP | D | 159 | 36.376 | 41.545 | 29.629 | 1.00 | 176.99 |
| 5874 | CB | ASP | D | 159 | 36.361 | 40.068 | 30.005 | 1.00 | 232.53 |
| 5875 | CG | ASP | D | 159 | 35.012 | 39.424 | 29.781 | 1.00 | 232.53 |
| 5876 | OD1 | ASP | D | 159 | 34.036 | 39.849 | 30.434 | 1.00 | 232.53 |
| 5877 | OD2 | ASP | D | 159 | 34.929 | 38.495 | 28.950 | 1.00 | 232.53 |
| 5878 | C | ASP | D | 159 | 37.805 | 42.075 | 29.760 | 1.00 | 176.99 |
| 5879 | O | ASP | D | 159 | 38.590 | 42.025 | 28.810 | 1.00 | 176.99 |
| 5880 | N | TYR | D | 160 | 38.143 | 42.588 | 30.938 | 1.00 | 175.60 |
| 5881 | CA | TYR | D | 160 | 39.484 | 43.102 | 31.170 | 1.00 | 175.60 |
| 5882 | CB | TYR | D | 160 | 39.559 | 44.592 | 30.873 | 1.00 | 205.83 |
| 5883 | CG | TYR | D | 160 | 39.112 | 44.956 | 29.483 | 1.00 | 205.83 |
| 5884 | CD1 | TYR | D | 160 | 37.778 | 45.271 | 29.216 | 1.00 | 205.83 |
| 5885 | CE1 | TYR | D | 160 | 37.361 | 45.636 | 27.939 | 1.00 | 205.83 |
| 5886 | CD2 | TYR | D | 160 | 40.022 | 45.009 | 28.435 | 1.00 | 205.83 |
| 5887 | CE2 | TYR | D | 160 | 39.614 | 45.373 | 27.148 | 1.00 | 205.83 |
| 5888 | CZ | TYR | D | 160 | 38.284 | 45.687 | 26.912 | 1.00 | 205.83 |
| 5889 | OH | TYR | D | 160 | 37.883 | 46.070 | 25.658 | 1.00 | 205.83 |
| 5890 | C | TYR | D | 160 | 39.941 | 42.855 | 32.593 | 1.00 | 175.60 |
| 5891 | O | TYR | D | 160 | 39.151 | 42.853 | 33.545 | 1.00 | 175.60 |
| 5892 | N | GLU | D | 161 | 41.243 | 42.653 | 32.718 | 1.00 | 144.68 |
| 5893 | CA | GLU | D | 161 | 41.879 | 42.385 | 33.998 | 1.00 | 144.68 |
| 5894 | CB | GLU | D | 161 | 42.697 | 41.094 | 33.859 | 1.00 | 232.05 |
| 5895 | CG | GLU | D | 161 | 43.497 | 40.642 | 35.071 | 1.00 | 232.05 |
| 5896 | CD | GLU | D | 161 | 43.969 | 39.194 | 34.942 | 1.00 | 232.05 |
| 5897 | OE1 | GLU | D | 161 | 44.936 | 38.819 | 35.643 | 1.00 | 232.05 |
| 5898 | OE2 | GLU | D | 161 | 43.363 | 38.430 | 34.151 | 1.00 | 232.05 |
| 5899 | C | GLU | D | 161 | 42.759 | 43.587 | 34.344 | 1.00 | 144.68 |
| 5900 | O | GLU | D | 161 | 43.353 | 44.206 | 33.459 | 1.00 | 144.68 |
| 5901 | N | SER | D | 162 | 42.814 | 43.922 | 35.628 | 1.00 | 134.82 |
| 5902 | CA | SER | D | 162 | 43.594 | 45.064 | 36.101 | 1.00 | 134.82 |
| 5903 | CB | SER | D | 162 | 42.881 | 45.712 | 37.288 | 1.00 | 129.75 |
| 5904 | OG | SER | D | 162 | 42.767 | 44.801 | 38.381 | 1.00 | 129.75 |
| 5905 | C | SER | D | 162 | 44.983 | 44.669 | 36.541 | 1.00 | 134.82 |
| 5906 | O | SER | D | 162 | 45.221 | 43.504 | 36.838 | 1.00 | 134.82 |
| 5907 | N | GLU | D | 163 | 45.898 | 45.634 | 36.581 | 1.00 | 145.43 |
| 5908 | CA | GLU | D | 163 | 47.238 | 45.334 | 37.050 | 1.00 | 145.43 |
| 5909 | CB | GLU | D | 163 | 48.133 | 46.575 | 36.964 | 1.00 | 249.69 |
| 5910 | CG | GLU | D | 163 | 48.587 | 46.949 | 35.552 | 1.00 | 249.69 |
| 5911 | CD | GLU | D | 163 | 49.651 | 46.008 | 35.001 | 1.00 | 249.69 |
| 5912 | OE1 | GLU | D | 163 | 50.709 | 45.850 | 35.650 | 1.00 | 249.69 |
| 5913 | OE2 | GLU | D | 163 | 49.433 | 45.429 | 33.917 | 1.00 | 249.69 |
| 5914 | C | GLU | D | 163 | 47.047 | 44.921 | 38.519 | 1.00 | 145.43 |
| 5915 | O | GLU | D | 163 | 46.101 | 45.384 | 39.168 | 1.00 | 145.43 |
| 5916 | N | PRO | D | 164 | 47.906 | 44.037 | 39.057 | 1.00 | 113.31 |
| 5917 | CD | PRO | D | 164 | 48.999 | 43.317 | 38.369 | 1.00 | 144.09 |
| 5918 | CA | PRO | D | 164 | 47.794 | 43.578 | 40.447 | 1.00 | 113.31 |
| 5919 | CB | PRO | D | 164 | 48.555 | 42.277 | 40.434 | 1.00 | 144.09 |
| 5920 | CG | PRO | D | 164 | 49.685 | 42.607 | 39.519 | 1.00 | 144.09 |
| 5921 | C | PRO | D | 164 | 48.395 | 44.576 | 41.422 | 1.00 | 113.31 |
| 5922 | O | PRO | D | 164 | 49.399 | 45.229 | 41.095 | 1.00 | 113.31 |
| 5923 | N | LEU | D | 165 | 47.807 | 44.679 | 42.613 | 1.00 | 104.79 |
| 5924 | CA | LEU | D | 165 | 48.305 | 45.642 | 43.591 | 1.00 | 104.79 |
| 5925 | CB | LEU | D | 165 | 47.329 | 46.816 | 43.687 | 1.00 | 127.61 |
| 5926 | CG | LEU | D | 165 | 47.719 | 47.920 | 44.665 | 1.00 | 127.61 |
| 5927 | CD1 | LEU | D | 165 | 49.250 | 48.130 | 44.649 | 1.00 | 127.61 |
| 5928 | CD2 | LEU | D | 165 | 46.959 | 49.193 | 44.283 | 1.00 | 127.61 |
| 5929 | C | LEU | D | 165 | 48.560 | 45.097 | 44.980 | 1.00 | 104.79 |
| 5930 | O | LEU | D | 165 | 47.691 | 44.427 | 45.545 | 1.00 | 104.79 |
| 5931 | N | ASN | D | 166 | 49.739 | 45.405 | 45.533 | 1.00 | 129.66 |
| 5932 | CA | ASN | D | 166 | 50.090 | 44.944 | 46.878 | 1.00 | 129.66 |
| 5933 | CB | ASN | D | 166 | 51.594 | 44.769 | 47.024 | 1.00 | 189.08 |
| 5934 | CG | ASN | D | 166 | 52.050 | 43.354 | 46.741 | 1.00 | 189.08 |
| 5935 | OD1 | ASN | D | 166 | 51.275 | 42.407 | 46.867 | 1.00 | 189.08 |
| 5936 | ND2 | ASN | D | 166 | 53.324 | 43.209 | 46.381 | 1.00 | 189.08 |
| 5937 | C | ASN | D | 166 | 49.612 | 45.924 | 47.955 | 1.00 | 129.66 |
| 5938 | O | ASN | D | 166 | 49.610 | 47.138 | 47.755 | 1.00 | 129.66 |
| 5939 | N | ILE | D | 167 | 49.221 | 45.387 | 49.105 | 1.00 | 126.98 |
| 5940 | CA | ILE | D | 167 | 48.731 | 46.196 | 50.211 | 1.00 | 126.98 |
| 5941 | CB | ILE | D | 167 | 47.211 | 46.242 | 50.220 | 1.00 | 113.09 |
| 5942 | CG2 | ILE | D | 167 | 46.740 | 46.998 | 51.438 | 1.00 | 113.09 |
| 5943 | CG1 | ILE | D | 167 | 46.716 | 46.889 | 48.940 | 1.00 | 113.09 |
| 5944 | CD1 | ILE | D | 167 | 45.225 | 46.900 | 48.843 | 1.00 | 113.09 |
| 5945 | C | ILE | D | 167 | 49.185 | 45.645 | 51.555 | 1.00 | 126.98 |
| 5946 | O | ILE | D | 167 | 48.978 | 44.480 | 51.875 | 1.00 | 126.98 |
| 5947 | N | THR | D | 168 | 49.769 | 46.499 | 52.369 | 1.00 | 123.13 |
| 5948 | CA | THR | D | 168 | 50.238 | 46.029 | 53.647 | 1.00 | 123.13 |
| 5949 | CB | THR | D | 168 | 51.761 | 46.052 | 53.678 | 1.00 | 145.39 |
| 5950 | OG1 | THR | D | 168 | 52.253 | 45.258 | 52.593 | 1.00 | 145.39 |
| 5951 | CG2 | THR | D | 168 | 52.281 | 45.490 | 54.987 | 1.00 | 145.39 |
| 5952 | C | THR | D | 168 | 49.695 | 46.766 | 54.864 | 1.00 | 123.13 |
| 5953 | O | THR | D | 168 | 49.839 | 47.983 | 55.000 | 1.00 | 123.13 |
| 5954 | N | VAL | D | 169 | 49.061 | 46.004 | 55.748 | 1.00 | 129.26 |
| 5955 | CA | VAL | D | 169 | 48.501 | 46.536 | 56.981 | 1.00 | 129.26 |
| 5956 | CB | VAL | D | 169 | 47.067 | 45.982 | 57.209 | 1.00 | 119.28 |
| 5957 | CG1 | VAL | D | 169 | 46.683 | 46.096 | 58.653 | 1.00 | 119.28 |
| 5958 | CG2 | VAL | D | 169 | 46.065 | 46.769 | 56.377 | 1.00 | 119.28 |
| 5959 | C | VAL | D | 169 | 49.439 | 46.104 | 58.098 | 1.00 | 129.26 |
| 5960 | O | VAL | D | 169 | 49.525 | 44.914 | 58.419 | 1.00 | 129.26 |
| 5961 | N | ILE | D | 170 | 50.162 | 47.069 | 58.664 | 1.00 | 108.63 |
| 5962 | CA | ILE | D | 170 | 51.111 | 46.798 | 59.750 | 1.00 | 108.63 |
| 5963 | CB | ILE | D | 170 | 52.340 | 47.687 | 59.607 | 1.00 | 169.72 |
| 5964 | CG2 | ILE | D | 170 | 52.891 | 47.560 | 58.200 | 1.00 | 169.72 |
| 5965 | CG1 | ILE | D | 170 | 51.963 | 49.148 | 59.833 | 1.00 | 169.72 |
| 5966 | CD1 | ILE | D | 170 | 53.138 | 50.116 | 59.741 | 1.00 | 169.72 |
| 5967 | C | ILE | D | 170 | 50.448 | 47.058 | 61.111 | 1.00 | 108.63 |
| 5968 | O | ILE | D | 170 | 49.389 | 47.687 | 61.170 | 1.00 | 108.63 |
| 5969 | N | LYS | D | 171 | 51.044 | 46.600 | 62.206 | 1.00 | 180.64 |
| 5970 | CA | LYS | D | 171 | 50.427 | 46.811 | 63.518 | 1.00 | 180.64 |
| 5971 | CB | LYS | D | 171 | 50.095 | 45.460 | 64.130 | 1.00 | 216.97 |
| 5972 | CG | LYS | D | 171 | 51.300 | 44.550 | 64.204 | 1.00 | 216.97 |
| 5973 | CD | LYS | D | 171 | 50.911 | 43.084 | 64.180 | 1.00 | 216.97 |
| 5974 | CE | LYS | D | 171 | 49.997 | 42.721 | 65.338 | 1.00 | 216.97 |
| 5975 | NZ | LYS | D | 171 | 49.677 | 41.265 | 65.351 | 1.00 | 216.97 |
| 5976 | C | LYS | D | 171 | 51.277 | 47.623 | 64.496 | 1.00 | 180.64 |
| 5977 | O | LYS | D | 171 | 50.952 | 47.715 | 65.686 | 1.00 | 180.64 |
| 5978 | C1 | NAG | D | 221 | 40.588 | 68.345 | 34.460 | 1.00 | 249.69 |
| 5979 | C2 | NAG | D | 221 | 39.263 | 67.620 | 34.228 | 1.00 | 249.69 |
| 5980 | N2 | NAG | D | 221 | 39.503 | 66.190 | 34.156 | 1.00 | 249.69 |
| 5981 | C7 | NAG | D | 221 | 38.524 | 65.328 | 34.426 | 1.00 | 249.69 |
| 5982 | O7 | NAG | D | 221 | 37.379 | 65.680 | 34.734 | 1.00 | 249.69 |
| 5983 | C8 | NAG | D | 221 | 38.865 | 63.847 | 34.336 | 1.00 | 249.69 |
| 5984 | C3 | NAG | D | 221 | 38.607 | 68.107 | 32.935 | 1.00 | 249.69 |
| 5985 | O3 | NAG | D | 221 | 37.303 | 67.542 | 32.815 | 1.00 | 249.69 |
| 5986 | C4 | NAG | D | 221 | 38.508 | 69.645 | 32.882 | 1.00 | 249.69 |
| 5987 | O4 | NAG | D | 221 | 38.122 | 70.004 | 31.534 | 1.00 | 249.69 |
| 5988 | C5 | NAG | D | 221 | 39.874 | 70.288 | 33.238 | 1.00 | 249.69 |
| 5989 | O5 | NAG | D | 221 | 40.374 | 69.764 | 34.489 | 1.00 | 249.69 |
| 5990 | C6 | NAG | D | 221 | 39.806 | 71.797 | 33.398 | 1.00 | 249.69 |
| 5991 | O6 | NAG | D | 221 | 38.830 | 72.175 | 34.359 | 1.00 | 249.69 |
| 5992 | C1 | NAG | D | 222 | 37.598 | 71.265 | 31.271 | 1.00 | 249.69 |
| 5993 | C2 | NAG | D | 222 | 36.393 | 71.128 | 30.316 | 1.00 | 249.69 |
| 5994 | N2 | NAG | D | 222 | 35.353 | 70.322 | 30.940 | 1.00 | 249.69 |
| 5995 | C7 | NAG | D | 222 | 34.138 | 70.826 | 31.159 | 1.00 | 249.69 |
| 5996 | O7 | NAG | D | 222 | 33.821 | 71.980 | 30.854 | 1.00 | 249.69 |
| 5997 | C8 | NAG | D | 222 | 33.115 | 69.911 | 31.816 | 1.00 | 249.69 |
| 5998 | C3 | NAG | D | 222 | 36.853 | 70.476 | 28.991 | 1.00 | 249.69 |
| 5999 | O3 | NAG | D | 222 | 35.784 | 70.461 | 28.055 | 1.00 | 249.69 |
| 6000 | C4 | NAG | D | 222 | 38.047 | 71.236 | 28.392 | 1.00 | 249.69 |
| 6001 | O4 | NAG | D | 222 | 38.552 | 70.527 | 27.265 | 1.00 | 249.69 |
| 6002 | C5 | NAG | D | 222 | 39.161 | 71.402 | 29.445 | 1.00 | 249.69 |
| 6003 | O5 | NAG | D | 222 | 38.632 | 72.044 | 30.639 | 1.00 | 249.69 |
| 6004 | C6 | NAG | D | 222 | 40.342 | 72.235 | 28.961 | 1.00 | 249.69 |
| 6005 | O6 | NAG | D | 222 | 41.578 | 71.582 | 29.216 | 1.00 | 249.69 |
| 6006 | C1 | NAG | D | 242 | 60.393 | 61.563 | 38.161 | 1.00 | 217.32 |
| 6007 | C2 | NAG | D | 242 | 60.080 | 62.065 | 36.753 | 1.00 | 217.32 |
| 6008 | N2 | NAG | D | 242 | 59.542 | 63.410 | 36.776 | 1.00 | 217.32 |
| 6009 | C7 | NAG | D | 242 | 60.185 | 64.390 | 36.140 | 1.00 | 217.32 |
| 6010 | O7 | NAG | D | 242 | 61.243 | 64.215 | 35.527 | 1.00 | 217.32 |
| 6011 | C8 | NAG | D | 242 | 59.572 | 65.781 | 36.193 | 1.00 | 217.32 |
| 6012 | C3 | NAG | D | 242 | 59.090 | 61.109 | 36.102 | 1.00 | 217.32 |
| 6013 | O3 | NAG | D | 242 | 58.789 | 61.543 | 34.778 | 1.00 | 217.32 |
| 6014 | C4 | NAG | D | 242 | 59.683 | 59.697 | 36.072 | 1.00 | 217.32 |
| 6015 | O4 | NAG | D | 242 | 58.682 | 58.764 | 35.604 | 1.00 | 217.32 |
| 6016 | C5 | NAG | D | 242 | 60.173 | 59.270 | 37.475 | 1.00 | 217.32 |
| 6017 | O5 | NAG | D | 242 | 61.023 | 60.283 | 38.071 | 1.00 | 217.32 |
| 6018 | C6 | NAG | D | 242 | 61.001 | 57.999 | 37.419 | 1.00 | 217.32 |
| 6019 | O6 | NAG | D | 242 | 60.329 | 56.906 | 38.029 | 1.00 | 217.32 |
| 6020 | C1 | NAG | D | 243 | 58.975 | 58.047 | 34.449 | 1.00 | 249.32 |
| 6021 | C2 | NAG | D | 243 | 58.093 | 56.797 | 34.373 | 1.00 | 249.32 |
| 6022 | N2 | NAG | D | 243 | 58.304 | 55.934 | 35.517 | 1.00 | 249.32 |
| 6023 | C7 | NAG | D | 243 | 57.260 | 55.458 | 36.184 | 1.00 | 249.32 |
| 6024 | O7 | NAG | D | 243 | 56.095 | 55.727 | 35.887 | 1.00 | 249.32 |
| 6025 | C8 | NAG | D | 243 | 57.553 | 54.550 | 37.368 | 1.00 | 249.32 |
| 6026 | C3 | NAG | D | 243 | 58.410 | 56.048 | 33.091 | 1.00 | 249.32 |
| 6027 | O3 | NAG | D | 243 | 57.609 | 54.878 | 32.999 | 1.00 | 249.32 |
| 6028 | C4 | NAG | D | 243 | 58.125 | 56.960 | 31.920 | 1.00 | 249.32 |
| 6029 | O4 | NAG | D | 243 | 58.387 | 56.198 | 30.758 | 1.00 | 249.32 |
| 6030 | C5 | NAG | D | 243 | 58.994 | 58.245 | 32.040 | 1.00 | 249.32 |
| 6031 | O5 | NAG | D | 243 | 58.710 | 58.893 | 33.315 | 1.00 | 249.32 |
| 6032 | C6 | NAG | D | 243 | 58.695 | 59.274 | 30.969 | 1.00 | 249.32 |
| 6033 | O6 | NAG | D | 243 | 57.361 | 59.747 | 31.063 | 1.00 | 249.32 |
| 6034 | C1 | MAN | D | 244 | 57.701 | 56.400 | 29.591 | 1.00 | 249.69 |
| 6035 | C2 | MAN | D | 244 | 58.764 | 56.236 | 28.599 | 1.00 | 249.69 |
| 6036 | O2 | MAN | D | 244 | 59.572 | 55.101 | 28.964 | 1.00 | 249.69 |
| 6037 | C3 | MAN | D | 244 | 58.183 | 56.214 | 27.213 | 1.00 | 249.69 |
| 6038 | O3 | MAN | D | 244 | 59.205 | 56.198 | 26.236 | 1.00 | 249.69 |
| 6039 | C4 | MAN | D | 244 | 57.187 | 55.086 | 27.057 | 1.00 | 249.69 |
| 6040 | O4 | MAN | D | 244 | 56.690 | 55.063 | 25.730 | 1.00 | 249.69 |
| 6041 | C5 | MAN | D | 244 | 56.059 | 55.334 | 28.066 | 1.00 | 249.69 |
| 6042 | O5 | MAN | D | 244 | 56.637 | 55.331 | 29.433 | 1.00 | 249.69 |
| 6043 | C6 | MAN | D | 244 | 54.855 | 54.371 | 27.914 | 1.00 | 249.69 |
| 6044 | O6 | MAN | D | 244 | 55.056 | 53.129 | 28.567 | 1.00 | 249.69 |
| 6045 | C1 | NAG | D | 250 | 45.970 | 78.192 | 45.348 | 1.00 | 249.69 |
| 6046 | C2 | NAG | D | 250 | 44.549 | 78.482 | 45.867 | 1.00 | 249.69 |
| 6047 | N2 | NAG | D | 250 | 44.538 | 78.485 | 47.317 | 1.00 | 249.69 |
| 6048 | C7 | NAG | D | 250 | 44.384 | 79.627 | 47.981 | 1.00 | 249.69 |
| 6049 | O7 | NAG | D | 250 | 44.241 | 80.713 | 47.415 | 1.00 | 249.69 |
| 6050 | C8 | NAG | D | 250 | 44.386 | 79.553 | 49.506 | 1.00 | 249.69 |
| 6051 | C3 | NAG | D | 250 | 43.581 | 77.413 | 45.337 | 1.00 | 249.69 |
| 6052 | O3 | NAG | D | 250 | 42.249 | 77.716 | 45.732 | 1.00 | 249.69 |
| 6053 | C4 | NAG | D | 250 | 43.666 | 77.341 | 43.807 | 1.00 | 249.69 |
| 6054 | O4 | NAG | D | 250 | 42.863 | 76.265 | 43.339 | 1.00 | 249.69 |
| 6055 | C5 | NAG | D | 250 | 45.136 | 77.138 | 43.368 | 1.00 | 249.69 |
| 6056 | O5 | NAG | D | 250 | 45.975 | 78.187 | 43.916 | 1.00 | 249.69 |
| 6057 | C6 | NAG | D | 250 | 45.334 | 77.155 | 41.856 | 1.00 | 249.69 |
| 6058 | O6 | NAG | D | 250 | 46.706 | 77.343 | 41.513 | 1.00 | 249.69 |
| 6059 | C1 | NAG | D | 274 | 64.018 | 69.436 | 61.817 | 1.00 | 249.69 |
| 6060 | C2 | NAG | D | 274 | 63.805 | 68.308 | 62.845 | 1.00 | 249.69 |
| 6061 | N2 | NAG | D | 274 | 62.614 | 68.567 | 63.639 | 1.00 | 249.69 |
| 6062 | C7 | NAG | D | 274 | 61.945 | 67.559 | 64.201 | 1.00 | 249.69 |
| 6063 | O7 | NAG | D | 274 | 62.289 | 66.377 | 64.093 | 1.00 | 249.69 |
| 6064 | C8 | NAG | D | 274 | 60.707 | 67.911 | 65.011 | 1.00 | 249.69 |
| 6065 | C3 | NAG | D | 274 | 65.040 | 68.194 | 63.760 | 1.00 | 249.69 |
| 6066 | O3 | NAG | D | 274 | 64.908 | 67.066 | 64.619 | 1.00 | 249.69 |
| 6067 | C4 | NAG | D | 274 | 66.321 | 68.053 | 62.922 | 1.00 | 249.69 |
| 6068 | O4 | NAG | D | 274 | 67.463 | 68.083 | 63.776 | 1.00 | 249.69 |
| 6069 | C5 | NAG | D | 274 | 66.405 | 69.191 | 61.890 | 1.00 | 249.69 |
| 6070 | O5 | NAG | D | 274 | 65.217 | 69.199 | 61.060 | 1.00 | 249.69 |
| 6071 | C6 | NAG | D | 274 | 67.605 | 69.054 | 60.964 | 1.00 | 249.69 |
| 6072 | O6 | NAG | D | 274 | 67.558 | 70.006 | 59.911 | 1.00 | 249.69 |
| 6073 | C1 | NAG | D | 335 | 33.933 | 54.753 | 43.517 | 1.00 | 249.69 |
| 6074 | C2 | NAG | D | 335 | 33.681 | 55.966 | 44.462 | 1.00 | 249.69 |
| 6075 | N2 | NAG | D | 335 | 33.369 | 55.476 | 45.797 | 1.00 | 249.69 |
| 6076 | C7 | NAG | D | 335 | 34.175 | 55.736 | 46.826 | 1.00 | 249.69 |
| 6077 | O7 | NAG | D | 335 | 35.208 | 56.398 | 46.727 | 1.00 | 249.69 |
| 6078 | C8 | NAG | D | 335 | 33.768 | 55.178 | 48.177 | 1.00 | 249.69 |
| 6079 | C3 | NAG | D | 335 | 32.547 | 56.909 | 44.003 | 1.00 | 249.69 |
| 6080 | O3 | NAG | D | 335 | 32.693 | 58.170 | 44.644 | 1.00 | 249.69 |
| 6081 | C4 | NAG | D | 335 | 32.568 | 57.114 | 42.494 | 1.00 | 249.69 |
| 6082 | O4 | NAG | D | 335 | 31.469 | 57.925 | 42.098 | 1.00 | 249.69 |
| 6083 | C5 | NAG | D | 335 | 32.490 | 55.747 | 41.830 | 1.00 | 249.69 |
| 6084 | O5 | NAG | D | 335 | 33.699 | 55.014 | 42.109 | 1.00 | 249.69 |
| 6085 | C6 | NAG | D | 335 | 32.365 | 55.844 | 40.319 | 1.00 | 249.69 |
| 6086 | O6 | NAG | D | 335 | 31.232 | 55.131 | 39.850 | 1.00 | 249.69 |
| 6087 | C1 | NAG | D | 340 | 38.129 | 47.005 | 65.199 | 1.00 | 249.69 |
| 6088 | C2 | NAG | D | 340 | 39.319 | 46.805 | 66.150 | 1.00 | 249.69 |
| 6089 | N2 | NAG | D | 340 | 40.524 | 46.521 | 65.388 | 1.00 | 249.69 |
| 6090 | C7 | NAG | D | 340 | 41.665 | 47.160 | 65.655 | 1.00 | 249.69 |
| 6091 | O7 | NAG | D | 340 | 41.779 | 48.007 | 66.549 | 1.00 | 249.69 |
| 6092 | C8 | NAG | D | 340 | 42.871 | 46.799 | 64.801 | 1.00 | 249.69 |
| 6093 | C3 | NAG | D | 340 | 39.000 | 45.640 | 67.106 | 1.00 | 249.69 |
| 6094 | O3 | NAG | D | 340 | 40.042 | 45.482 | 68.064 | 1.00 | 249.69 |
| 6095 | C4 | NAG | D | 340 | 37.670 | 45.894 | 67.828 | 1.00 | 249.69 |
| 6096 | O4 | NAG | D | 340 | 37.324 | 44.743 | 68.593 | 1.00 | 249.69 |
| 6097 | C5 | NAG | D | 340 | 36.556 | 46.207 | 66.801 | 1.00 | 249.69 |
| 6098 | O5 | NAG | D | 340 | 36.949 | 47.307 | 65.948 | 1.00 | 249.69 |
| 6099 | C6 | NAG | D | 340 | 35.226 | 46.591 | 67.427 | 1.00 | 249.69 |
| 6100 | O6 | NAG | D | 340 | 34.319 | 47.067 | 66.440 | 1.00 | 249.69 |
| 6101 | C1 | NAG | D | 366 | 53.829 | 41.917 | 45.964 | 1.00 | 214.56 |
| 6102 | C2 | NAG | D | 366 | 54.811 | 42.093 | 44.812 | 1.00 | 214.56 |
| 6103 | N2 | NAG | D | 366 | 54.141 | 42.757 | 43.705 | 1.00 | 214.56 |
| 6104 | C7 | NAG | D | 366 | 54.172 | 44.086 | 43.595 | 1.00 | 214.56 |
| 6105 | O7 | NAG | D | 366 | 54.769 | 44.819 | 44.396 | 1.00 | 214.56 |
| 6106 | C8 | NAG | D | 366 | 53.436 | 44.705 | 42.413 | 1.00 | 214.56 |
| 6107 | C3 | NAG | D | 366 | 55.328 | 40.725 | 44.367 | 1.00 | 214.56 |
| 6108 | O3 | NAG | D | 366 | 56.374 | 40.896 | 43.416 | 1.00 | 214.56 |
| 6109 | C4 | NAG | D | 366 | 55.847 | 39.896 | 45.553 | 1.00 | 214.56 |
| 6110 | O4 | NAG | D | 366 | 56.067 | 38.537 | 45.104 | 1.00 | 214.56 |
| 6111 | C5 | NAG | D | 366 | 54.830 | 39.900 | 46.709 | 1.00 | 214.56 |
| 6112 | O5 | NAG | D | 366 | 54.471 | 41.245 | 47.050 | 1.00 | 214.56 |
| 6113 | C6 | NAG | D | 366 | 55.334 | 39.248 | 47.980 | 1.00 | 214.56 |
| 6114 | O6 | NAG | D | 366 | 54.292 | 39.118 | 48.934 | 1.00 | 214.56 |
| 6115 | C1 | NAG | D | 367 | 57.323 | 37.987 | 45.329 | 1.00 | 231.83 |
| 6116 | C2 | NAG | D | 367 | 57.238 | 36.462 | 45.283 | 1.00 | 231.83 |
| 6117 | N2 | NAG | D | 367 | 56.271 | 35.974 | 46.246 | 1.00 | 231.83 |
| 6118 | C7 | NAG | D | 367 | 55.141 | 35.410 | 45.821 | 1.00 | 231.83 |
| 6119 | O7 | NAG | D | 367 | 54.861 | 35.279 | 44.620 | 1.00 | 231.83 |
| 6120 | C8 | NAG | D | 367 | 54.169 | 34.918 | 46.887 | 1.00 | 231.83 |
| 6121 | C3 | NAG | D | 367 | 58.627 | 35.898 | 45.572 | 1.00 | 231.83 |
| 6122 | O3 | NAG | D | 367 | 58.601 | 34.478 | 45.528 | 1.00 | 231.83 |
| 6123 | C4 | NAG | D | 367 | 59.611 | 36.433 | 44.526 | 1.00 | 231.83 |
| 6124 | O4 | NAG | D | 367 | 60.922 | 35.989 | 44.845 | 1.00 | 231.83 |
| 6125 | C5 | NAG | D | 367 | 59.572 | 37.974 | 44.486 | 1.00 | 231.83 |
| 6126 | O5 | NAG | D | 367 | 58.216 | 38.443 | 44.296 | 1.00 | 231.83 |
| 6127 | C6 | NAG | D | 367 | 60.403 | 38.550 | 43.358 | 1.00 | 231.83 |
| 6128 | O6 | NAG | D | 367 | 59.584 | 39.177 | 42.385 | 1.00 | 231.83 |
| 6129 | CB | LYS | E | 4 | 8.883 | 64.586 | 0.000 | 1.00 | 249.69 |
| 6130 | CG | LYS | E | 4 | 7.510 | 64.141 | −0.503 | 1.00 | 249.69 |
| 6131 | CD | LYS | E | 4 | 6.532 | 63.873 | 0.645 | 1.00 | 249.69 |
| 6132 | CE | LYS | E | 4 | 5.149 | 63.459 | 0.123 | 1.00 | 249.69 |
| 6133 | NZ | LYS | E | 4 | 4.173 | 63.179 | 1.220 | 1.00 | 249.69 |
| 6134 | C | LYS | E | 4 | 9.271 | 66.045 | −1.989 | 1.00 | 232.34 |
| 6135 | O | LYS | E | 4 | 8.420 | 66.812 | −1.537 | 1.00 | 232.34 |
| 6136 | N | LYS | E | 4 | 11.173 | 65.351 | −0.540 | 1.00 | 232.34 |
| 6137 | CA | LYS | E | 4 | 9.865 | 64.939 | −1.121 | 1.00 | 232.34 |
| 6138 | N | PRO | E | 5 | 9.723 | 66.150 | −3.249 | 1.00 | 227.45 |
| 6139 | CD | PRO | E | 5 | 10.890 | 65.481 | −3.843 | 1.00 | 124.73 |
| 6140 | CA | PRO | E | 5 | 9.204 | 67.180 | −4.150 | 1.00 | 227.45 |
| 6141 | CB | PRO | E | 5 | 10.351 | 67.382 | −5.132 | 1.00 | 124.73 |
| 6142 | CG | PRO | E | 5 | 10.883 | 66.000 | −5.275 | 1.00 | 124.73 |
| 6143 | C | PRO | E | 5 | 7.921 | 66.721 | −4.845 | 1.00 | 227.45 |
| 6144 | O | PRO | E | 5 | 7.651 | 65.522 | −4.954 | 1.00 | 227.45 |
| 6145 | N | LYS | E | 6 | 7.125 | 67.679 | −5.305 | 1.00 | 237.93 |
| 6146 | CA | LYS | E | 6 | 5.877 | 67.360 | −5.987 | 1.00 | 237.93 |
| 6147 | CB | LYS | E | 6 | 4.702 | 67.440 | −5.011 | 1.00 | 249.69 |
| 6148 | CG | LYS | E | 6 | 3.370 | 67.066 | −5.640 | 1.00 | 249.69 |
| 6149 | CD | LYS | E | 6 | 2.244 | 66.993 | −4.615 | 1.00 | 249.69 |
| 6150 | CE | LYS | E | 6 | 0.935 | 66.562 | −5.275 | 1.00 | 249.69 |
| 6151 | NZ | LYS | E | 6 | −0.156 | 66.352 | −4.287 | 1.00 | 249.69 |
| 6152 | C | LYS | E | 6 | 5.650 | 68.308 | −7.153 | 1.00 | 237.93 |
| 6153 | O | LYS | E | 6 | 5.422 | 69.505 | −6.970 | 1.00 | 237.93 |
| 6154 | N | VAL | E | 7 | 5.709 | 67.754 | −8.356 | 1.00 | 162.26 |
| 6155 | CA | VAL | E | 7 | 5.532 | 68.543 | −9.567 | 1.00 | 162.26 |
| 6156 | CB | VAL | E | 7 | 5.858 | 67.703 | −10.821 | 1.00 | 205.67 |
| 6157 | CG1 | VAL | E | 7 | 6.017 | 68.614 | −12.040 | 1.00 | 205.67 |
| 6158 | CG2 | VAL | E | 7 | 7.117 | 66.881 | −10.577 | 1.00 | 205.67 |
| 6159 | C | VAL | E | 7 | 4.118 | 69.111 | −9.723 | 1.00 | 162.26 |
| 6160 | O | VAL | E | 7 | 3.134 | 68.364 | −9.717 | 1.00 | 162.26 |
| 6161 | N | SER | E | 8 | 4.022 | 70.433 | −9.868 | 1.00 | 174.49 |
| 6162 | CA | SER | E | 8 | 2.741 | 71.111 | −10.048 | 1.00 | 174.49 |
| 6163 | CB | SER | E | 8 | 2.672 | 72.324 | −9.131 | 1.00 | 223.27 |
| 6164 | OG | SER | E | 8 | 3.850 | 73.111 | −9.231 | 1.00 | 223.27 |
| 6165 | C | SER | E | 8 | 2.616 | 71.551 | −11.501 | 1.00 | 174.49 |
| 6166 | O | SER | E | 8 | 3.624 | 71.640 | −12.215 | 1.00 | 174.49 |
| 6167 | N | LEU | E | 9 | 1.392 | 71.815 | −11.949 | 1.00 | 138.24 |
| 6168 | CA | LEU | E | 9 | 1.200 | 72.248 | −13.338 | 1.00 | 138.24 |
| 6169 | CB | LEU | E | 9 | 0.497 | 71.161 | −14.181 | 1.00 | 151.07 |
| 6170 | CG | LEU | E | 9 | 1.092 | 69.761 | −14.372 | 1.00 | 151.07 |
| 6171 | CD1 | LEU | E | 9 | 0.374 | 69.105 | −15.529 | 1.00 | 151.07 |
| 6172 | CD2 | LEU | E | 9 | 2.578 | 69.816 | −14.665 | 1.00 | 151.07 |
| 6173 | C | LEU | E | 9 | 0.394 | 73.544 | −13.447 | 1.00 | 138.24 |
| 6174 | O | LEU | E | 9 | −0.329 | 73.919 | −12.519 | 1.00 | 138.24 |
| 6175 | N | ASN | E | 10 | 0.520 | 74.217 | −14.589 | 1.00 | 163.51 |
| 6176 | CA | ASN | E | 10 | −0.215 | 75.436 | −14.823 | 1.00 | 163.51 |
| 6177 | CB | ASN | E | 10 | 0.444 | 76.592 | −14.100 | 1.00 | 242.89 |
| 6178 | CG | ASN | E | 10 | −0.467 | 77.788 | −14.008 | 1.00 | 242.89 |
| 6179 | OD1 | ASN | E | 10 | −1.520 | 77.730 | −13.369 | 1.00 | 242.89 |
| 6180 | ND2 | ASN | E | 10 | −0.077 | 78.881 | −14.650 | 1.00 | 242.89 |
| 6181 | C | ASN | E | 10 | −0.303 | 75.748 | −16.310 | 1.00 | 163.51 |
| 6182 | O | ASN | E | 10 | 0.703 | 76.073 | −16.942 | 1.00 | 163.51 |
| 6183 | N | PRO | E | 11 | −1.515 | 75.661 | −16.902 | 1.00 | 167.75 |
| 6184 | CD | PRO | E | 11 | −1.686 | 75.947 | −18.332 | 1.00 | 141.15 |
| 6185 | CA | PRO | E | 11 | −2.807 | 75.308 | −16.289 | 1.00 | 167.75 |
| 6186 | CB | PRO | E | 11 | −3.740 | 75.243 | −17.494 | 1.00 | 141.15 |
| 6187 | CG | PRO | E | 11 | −3.151 | 76.257 | −18.413 | 1.00 | 141.15 |
| 6188 | C | PRO | E | 11 | −2.804 | 73.994 | −15.505 | 1.00 | 167.75 |
| 6189 | O | PRO | E | 11 | −1.861 | 73.210 | −15.588 | 1.00 | 167.75 |
| 6190 | N | PRO | E | 12 | −3.874 | 73.736 | −14.729 | 1.00 | 129.01 |
| 6191 | CD | PRO | E | 12 | −5.008 | 74.630 | −14.438 | 1.00 | 158.46 |
| 6192 | CA | PRO | E | 12 | −3.963 | 72.506 | −13.934 | 1.00 | 129.01 |
| 6193 | CB | PRO | E | 12 | −5.164 | 72.766 | −13.028 | 1.00 | 158.46 |
| 6194 | CG | PRO | E | 12 | −5.297 | 74.281 | −13.015 | 1.00 | 158.46 |
| 6195 | C | PRO | E | 12 | −4.198 | 71.307 | −14.852 | 1.00 | 129.01 |
| 6196 | O | PRO | E | 12 | −3.823 | 70.178 | −14.535 | 1.00 | 129.01 |
| 6197 | N | TRP | E | 13 | −4.829 | 71.590 | −15.992 | 1.00 | 148.97 |
| 6198 | CA | TRP | E | 13 | −5.176 | 70.617 | −17.041 | 1.00 | 148.97 |
| 6199 | CB | TRP | E | 13 | −5.707 | 71.376 | −18.252 | 1.00 | 139.57 |
| 6200 | CG | TRP | E | 13 | −6.745 | 72.405 | −17.878 | 1.00 | 139.57 |
| 6201 | CD2 | TRP | E | 13 | −7.685 | 72.310 | −16.810 | 1.00 | 139.57 |
| 6202 | CE2 | TRP | E | 13 | −8.485 | 73.479 | −16.851 | 1.00 | 139.57 |
| 6203 | CE3 | TRP | E | 13 | −7.934 | 71.351 | −15.816 | 1.00 | 139.57 |
| 6204 | CD1 | TRP | E | 13 | −7.006 | 73.598 | −18.513 | 1.00 | 139.57 |
| 6205 | NE1 | TRP | E | 13 | −8.049 | 74.249 | −17.900 | 1.00 | 139.57 |
| 6206 | CZ2 | TRP | E | 13 | −9.511 | 73.707 | −15.934 | 1.00 | 139.57 |
| 6207 | CZ3 | TRP | E | 13 | −8.950 | 71.581 | −14.909 | 1.00 | 139.57 |
| 6208 | CH2 | TRP | E | 13 | −9.731 | 72.746 | −14.975 | 1.00 | 139.57 |
| 6209 | C | TRP | E | 13 | −4.000 | 69.772 | −17.478 | 1.00 | 148.97 |
| 6210 | O | TRP | E | 13 | −3.050 | 70.294 | −18.052 | 1.00 | 148.97 |
| 6211 | N | ASN | E | 14 | −4.069 | 68.465 | −17.236 | 1.00 | 121.67 |
| 6212 | CA | ASN | E | 14 | −2.967 | 67.569 | −17.623 | 1.00 | 121.67 |
| 6213 | CB | ASN | E | 14 | −2.569 | 66.659 | −16.443 | 1.00 | 170.43 |
| 6214 | CG | ASN | E | 14 | −3.669 | 65.717 | −16.039 | 1.00 | 170.43 |
| 6215 | OD1 | ASN | E | 14 | −4.785 | 66.134 | −15.711 | 1.00 | 170.43 |
| 6216 | ND2 | ASN | E | 14 | −3.362 | 64.432 | −16.058 | 1.00 | 170.43 |
| 6217 | C | ASN | E | 14 | −3.273 | 66.730 | −18.871 | 1.00 | 121.67 |
| 6218 | O | ASN | E | 14 | −2.662 | 65.684 | −19.103 | 1.00 | 121.67 |
| 6219 | N | ARG | E | 15 | −4.227 | 67.225 | −19.661 | 1.00 | 100.94 |
| 6220 | CA | ARG | E | 15 | −4.675 | 66.616 | −20.914 | 1.00 | 100.94 |
| 6221 | CB | ARG | E | 15 | −6.069 | 65.991 | −20.773 | 1.00 | 110.25 |
| 6222 | CG | ARG | E | 15 | −6.278 | 65.052 | −19.581 | 1.00 | 110.25 |
| 6223 | CD | ARG | E | 15 | −7.642 | 64.353 | −19.673 | 1.00 | 110.25 |
| 6224 | NE | ARG | E | 15 | −7.673 | 63.268 | −20.667 | 1.00 | 110.25 |
| 6225 | CZ | ARG | E | 15 | −8.713 | 62.965 | −21.449 | 1.00 | 110.25 |
| 6226 | NH1 | ARG | E | 15 | −9.818 | 63.665 | −21.363 | 1.00 | 110.25 |
| 6227 | NH2 | ARG | E | 15 | −8.654 | 61.949 | −22.308 | 1.00 | 110.25 |
| 6228 | C | ARG | E | 15 | −4.797 | 67.800 | −21.845 | 1.00 | 100.94 |
| 6229 | O | ARG | E | 15 | −5.779 | 68.526 | −21.776 | 1.00 | 100.94 |
| 6230 | N | ILE | E | 16 | −3.824 | 68.012 | −22.720 | 1.00 | 114.46 |
| 6231 | CA | ILE | E | 16 | −3.875 | 69.182 | −23.615 | 1.00 | 114.46 |
| 6232 | CB | ILE | E | 16 | −2.723 | 70.141 | −23.315 | 1.00 | 133.29 |
| 6233 | CG2 | ILE | E | 16 | −3.008 | 70.920 | −22.025 | 1.00 | 133.29 |
| 6234 | CG1 | ILE | E | 16 | −1.422 | 69.334 | −23.257 | 1.00 | 133.29 |
| 6235 | CD1 | ILE | E | 16 | −0.187 | 70.169 | −23.241 | 1.00 | 133.29 |
| 6236 | C | ILE | E | 16 | −3.835 | 68.931 | −25.116 | 1.00 | 114.46 |
| 6237 | O | ILE | E | 16 | −3.314 | 67.920 | −25.573 | 1.00 | 114.46 |
| 6238 | N | PHE | E | 17 | −4.371 | 69.884 | −25.869 | 1.00 | 157.55 |
| 6239 | CA | PHE | E | 17 | −4.389 | 69.799 | −27.315 | 1.00 | 157.55 |
| 6240 | CB | PHE | E | 17 | −5.291 | 70.875 | −27.896 | 1.00 | 134.86 |
| 6241 | CG | PHE | E | 17 | −6.708 | 70.444 | −28.068 | 1.00 | 134.86 |
| 6242 | CD1 | PHE | E | 17 | −7.747 | 71.354 | −27.884 | 1.00 | 134.86 |
| 6243 | CD2 | PHE | E | 17 | −7.016 | 69.142 | −28.445 | 1.00 | 134.86 |
| 6244 | CE1 | PHE | E | 17 | −9.085 | 70.974 | −28.072 | 1.00 | 134.86 |
| 6245 | CE2 | PHE | E | 17 | −8.339 | 68.753 | −28.636 | 1.00 | 134.86 |
| 6246 | CZ | PHE | E | 17 | −9.378 | 69.677 | −28.448 | 1.00 | 134.86 |
| 6247 | C | PHE | E | 17 | −2.984 | 69.975 | −27.873 | 1.00 | 157.55 |
| 6248 | O | PHE | E | 17 | −2.038 | 70.221 | −27.122 | 1.00 | 157.55 |
| 6249 | N | LYS | E | 18 | −2.860 | 69.858 | −29.196 | 1.00 | 136.71 |
| 6250 | CA | LYS | E | 18 | −1.582 | 69.998 | −29.892 | 1.00 | 136.71 |
| 6251 | CB | LYS | E | 18 | −1.660 | 69.250 | −31.219 | 1.00 | 249.69 |
| 6252 | CG | LYS | E | 18 | −0.384 | 69.249 | −32.033 | 1.00 | 249.69 |
| 6253 | CD | LYS | E | 18 | −0.530 | 68.316 | −33.230 | 1.00 | 249.69 |
| 6254 | CE | LYS | E | 18 | 0.701 | 68.337 | −34.128 | 1.00 | 249.69 |
| 6255 | NZ | LYS | E | 18 | 0.855 | 69.643 | −34.825 | 1.00 | 249.69 |
| 6256 | C | LYS | E | 18 | −1.243 | 71.471 | −30.127 | 1.00 | 136.71 |
| 6257 | O | LYS | E | 18 | −2.056 | 72.237 | −30.646 | 1.00 | 136.71 |
| 6258 | N | GLY | E | 19 | −0.042 | 71.866 | −29.726 | 1.00 | 201.41 |
| 6259 | CA | GLY | E | 19 | 0.380 | 73.242 | −29.913 | 1.00 | 201.41 |
| 6260 | C | GLY | E | 19 | 0.195 | 74.161 | −28.717 | 1.00 | 201.41 |
| 6261 | O | GLY | E | 19 | 0.717 | 75.279 | −28.708 | 1.00 | 201.41 |
| 6262 | N | GLU | E | 20 | −0.541 | 73.703 | −27.709 | 1.00 | 148.40 |
| 6263 | CA | GLU | E | 20 | −0.787 | 74.499 | −26.497 | 1.00 | 148.40 |
| 6264 | CB | GLU | E | 20 | −2.004 | 73.945 | −25.733 | 1.00 | 165.83 |
| 6265 | CG | GLU | E | 20 | −3.267 | 73.658 | −26.577 | 1.00 | 165.83 |
| 6266 | CD | GLU | E | 20 | −4.479 | 73.233 | −25.726 | 1.00 | 165.83 |
| 6267 | OE1 | GLU | E | 20 | −4.358 | 72.282 | −24.926 | 1.00 | 165.83 |
| 6268 | OE2 | GLU | E | 20 | −5.555 | 73.855 | −25.864 | 1.00 | 165.83 |
| 6269 | C | GLU | E | 20 | 0.439 | 74.469 | −25.564 | 1.00 | 148.40 |
| 6270 | O | GLU | E | 20 | 1.273 | 73.561 | −25.663 | 1.00 | 148.40 |
| 6271 | N | ASN | E | 21 | 0.552 | 75.440 | −24.653 | 1.00 | 156.11 |
| 6272 | CA | ASN | E | 21 | 1.704 | 75.474 | −23.731 | 1.00 | 156.11 |
| 6273 | CB | ASN | E | 21 | 2.412 | 76.835 | −23.774 | 1.00 | 249.69 |
| 6274 | CG | ASN | E | 21 | 2.414 | 77.468 | −25.153 | 1.00 | 249.69 |
| 6275 | OD1 | ASN | E | 21 | 2.734 | 76.825 | −26.157 | 1.00 | 249.69 |
| 6276 | ND2 | ASN | E | 21 | 2.070 | 78.754 | −25.179 | 1.00 | 249.69 |
| 6277 | C | ASN | E | 21 | 1.342 | 75.182 | −22.267 | 1.00 | 156.11 |
| 6278 | O | ASN | E | 21 | 0.288 | 75.589 | −21.769 | 1.00 | 156.11 |
| 6279 | N | VAL | E | 22 | 2.245 | 74.493 | −21.580 | 1.00 | 182.17 |
| 6280 | CA | VAL | E | 22 | 2.049 | 74.137 | −20.177 | 1.00 | 182.17 |
| 6281 | CB | VAL | E | 22 | 1.601 | 72.669 | −20.028 | 1.00 | 148.81 |
| 6282 | CG1 | VAL | E | 22 | 2.757 | 71.742 | −20.356 | 1.00 | 148.81 |
| 6283 | CG2 | VAL | E | 22 | 1.109 | 72.404 | −18.627 | 1.00 | 148.81 |
| 6284 | C | VAL | E | 22 | 3.360 | 74.313 | −19.407 | 1.00 | 182.17 |
| 6285 | O | VAL | E | 22 | 4.443 | 74.124 | −19.965 | 1.00 | 182.17 |
| 6286 | N | THR | E | 23 | 3.261 | 74.650 | −18.120 | 1.00 | 131.37 |
| 6287 | CA | THR | E | 23 | 4.447 | 74.874 | −17.281 | 1.00 | 131.37 |
| 6288 | CB | THR | E | 23 | 4.448 | 76.320 | −16.723 | 1.00 | 249.69 |
| 6289 | OG1 | THR | E | 23 | 4.251 | 77.255 | −17.794 | 1.00 | 249.69 |
| 6290 | CG2 | THR | E | 23 | 5.773 | 76.619 | −16.027 | 1.00 | 249.69 |
| 6291 | C | THR | E | 23 | 4.559 | 73.928 | −16.088 | 1.00 | 131.37 |
| 6292 | O | THR | E | 23 | 3.643 | 73.866 | −15.266 | 1.00 | 131.37 |
| 6293 | N | LEU | E | 24 | 5.694 | 73.234 | −15.974 | 1.00 | 247.08 |
| 6294 | CA | LEU | E | 24 | 5.909 | 72.305 | −14.862 | 1.00 | 247.08 |
| 6295 | CB | LEU | E | 24 | 6.490 | 70.972 | −15.357 | 1.00 | 141.93 |
| 6296 | CG | LEU | E | 24 | 6.154 | 70.407 | −16.745 | 1.00 | 141.93 |
| 6297 | CD1 | LEU | E | 24 | 6.601 | 68.946 | −16.814 | 1.00 | 141.93 |
| 6298 | CD2 | LEU | E | 24 | 4.668 | 70.513 | −17.025 | 1.00 | 141.93 |
| 6299 | C | LEU | E | 24 | 6.857 | 72.886 | −13.807 | 1.00 | 247.08 |
| 6300 | O | LEU | E | 24 | 8.064 | 72.988 | −14.032 | 1.00 | 247.08 |
| 6301 | N | THR | E | 25 | 6.302 | 73.254 | −12.653 | 1.00 | 187.47 |
| 6302 | CA | THR | E | 25 | 7.075 | 73.824 | −11.547 | 1.00 | 187.47 |
| 6303 | CB | THR | E | 25 | 6.280 | 74.957 | −10.847 | 1.00 | 161.19 |
| 6304 | OG1 | THR | E | 25 | 5.936 | 75.964 | −11.806 | 1.00 | 161.19 |
| 6305 | CG2 | THR | E | 25 | 7.100 | 75.590 | −9.725 | 1.00 | 161.19 |
| 6306 | C | THR | E | 25 | 7.391 | 72.739 | −10.512 | 1.00 | 187.47 |
| 6307 | O | THR | E | 25 | 6.513 | 71.981 | −10.121 | 1.00 | 187.47 |
| 6308 | N | CYS | E | 26 | 8.642 | 72.673 | −10.069 | 1.00 | 208.94 |
| 6309 | CA | CYS | E | 26 | 9.057 | 71.677 | −9.078 | 1.00 | 208.94 |
| 6310 | C | CYS | E | 26 | 8.680 | 72.137 | −7.667 | 1.00 | 208.94 |
| 6311 | O | CYS | E | 26 | 8.738 | 73.326 | −7.364 | 1.00 | 208.94 |
| 6312 | CB | CYS | E | 26 | 10.562 | 71.439 | −9.179 | 1.00 | 205.14 |
| 6313 | SG | CYS | E | 26 | 11.190 | 70.046 | −8.197 | 1.00 | 205.14 |
| 6314 | N | ASN | E | 27 | 8.303 | 71.189 | −6.812 | 1.00 | 249.69 |
| 6315 | CA | ASN | E | 27 | 7.875 | 71.478 | −5.439 | 1.00 | 249.69 |
| 6316 | CB | ASN | E | 27 | 8.415 | 70.418 | −4.468 | 1.00 | 249.69 |
| 6317 | CG | ASN | E | 27 | 7.792 | 70.526 | −3.076 | 1.00 | 249.69 |
| 6318 | OD1 | ASN | E | 27 | 6.569 | 70.623 | −2.936 | 1.00 | 249.69 |
| 6319 | ND2 | ASN | E | 27 | 8.632 | 70.505 | −2.043 | 1.00 | 249.69 |
| 6320 | C | ASN | E | 27 | 8.241 | 72.870 | −4.930 | 1.00 | 249.69 |
| 6321 | O | ASN | E | 27 | 9.333 | 73.095 | −4.401 | 1.00 | 249.69 |
| 6322 | N | GLY | E | 28 | 7.301 | 73.795 | −5.095 | 1.00 | 249.69 |
| 6323 | CA | GLY | E | 28 | 7.486 | 75.172 | −4.669 | 1.00 | 249.69 |
| 6324 | C | GLY | E | 28 | 6.306 | 75.960 | −5.202 | 1.00 | 249.69 |
| 6325 | O | GLY | E | 28 | 6.092 | 76.005 | −6.419 | 1.00 | 249.69 |
| 6326 | N | ASN | E | 29 | 5.537 | 76.576 | −4.305 | 1.00 | 249.69 |
| 6327 | CA | ASN | E | 29 | 4.349 | 77.334 | −4.702 | 1.00 | 249.69 |
| 6328 | CB | ASN | E | 29 | 3.447 | 77.573 | −3.470 | 1.00 | 249.69 |
| 6329 | CG | ASN | E | 29 | 2.043 | 78.092 | −3.837 | 1.00 | 249.69 |
| 6330 | OD1 | ASN | E | 29 | 1.664 | 78.143 | −5.013 | 1.00 | 249.69 |
| 6331 | ND2 | ASN | E | 29 | 1.268 | 78.469 | −2.821 | 1.00 | 249.69 |
| 6332 | C | ASN | E | 29 | 4.659 | 78.665 | −5.406 | 1.00 | 249.69 |
| 6333 | O | ASN | E | 29 | 4.147 | 78.925 | −6.509 | 1.00 | 249.69 |
| 6334 | N | ASN | E | 30 | 5.502 | 79.498 | −4.796 | 1.00 | 249.69 |
| 6335 | CA | ASN | E | 30 | 5.807 | 80.792 | −5.395 | 1.00 | 249.69 |
| 6336 | CB | ASN | E | 30 | 5.157 | 81.904 | −4.559 | 1.00 | 249.69 |
| 6337 | CG | ASN | E | 30 | 3.636 | 81.814 | −4.543 | 1.00 | 249.69 |
| 6338 | OD1 | ASN | E | 30 | 3.012 | 81.900 | −3.481 | 1.00 | 249.69 |
| 6339 | ND2 | ASN | E | 30 | 3.030 | 81.646 | −5.721 | 1.00 | 249.69 |
| 6340 | C | ASN | E | 30 | 7.288 | 81.109 | −5.624 | 1.00 | 249.69 |
| 6341 | O | ASN | E | 30 | 7.734 | 81.191 | −6.773 | 1.00 | 249.69 |
| 6342 | N | PHE | E | 31 | 8.047 | 81.290 | −4.544 | 1.00 | 249.25 |
| 6343 | CA | PHE | E | 31 | 9.464 | 81.634 | −4.672 | 1.00 | 249.25 |
| 6344 | CB | PHE | E | 31 | 9.744 | 82.979 | −3.973 | 1.00 | 249.69 |
| 6345 | CG | PHE | E | 31 | 8.853 | 84.109 | −4.448 | 1.00 | 249.69 |
| 6346 | CD1 | PHE | E | 31 | 7.546 | 84.234 | −3.971 | 1.00 | 249.69 |
| 6347 | CD2 | PHE | E | 31 | 9.307 | 85.030 | −5.397 | 1.00 | 249.69 |
| 6348 | CE1 | PHE | E | 31 | 6.702 | 85.255 | −4.433 | 1.00 | 249.69 |
| 6349 | CE2 | PHE | E | 31 | 8.470 | 86.054 | −5.864 | 1.00 | 249.69 |
| 6350 | CZ | PHE | E | 31 | 7.167 | 86.166 | −5.380 | 1.00 | 249.69 |
| 6351 | C | PHE | E | 31 | 10.439 | 80.568 | −4.166 | 1.00 | 249.25 |
| 6352 | O | PHE | E | 31 | 10.399 | 80.170 | −3.004 | 1.00 | 249.25 |
| 6353 | N | PHE | E | 32 | 11.324 | 80.127 | −5.061 | 1.00 | 241.74 |
| 6354 | CA | PHE | E | 32 | 12.329 | 79.106 | −4.757 | 1.00 | 241.74 |
| 6355 | CB | PHE | E | 32 | 12.131 | 77.894 | −5.677 | 1.00 | 249.69 |
| 6356 | CG | PHE | E | 32 | 12.858 | 76.648 | −5.224 | 1.00 | 249.69 |
| 6357 | CD1 | PHE | E | 32 | 12.489 | 75.993 | −4.048 | 1.00 | 249.69 |
| 6358 | CD2 | PHE | E | 32 | 13.905 | 76.121 | −5.984 | 1.00 | 249.69 |
| 6359 | CE1 | PHE | E | 32 | 13.150 | 74.834 | −3.639 | 1.00 | 249.69 |
| 6360 | CE2 | PHE | E | 32 | 14.570 | 74.962 | −5.582 | 1.00 | 249.69 |
| 6361 | CZ | PHE | E | 32 | 14.191 | 74.320 | −4.407 | 1.00 | 249.69 |
| 6362 | C | PHE | E | 32 | 13.743 | 79.667 | −4.926 | 1.00 | 241.74 |
| 6363 | O | PHE | E | 32 | 13.927 | 80.745 | −5.490 | 1.00 | 241.74 |
| 6364 | N | GLU | E | 33 | 14.739 | 78.916 | −4.465 | 1.00 | 249.60 |
| 6365 | CA | GLU | E | 33 | 16.114 | 79.383 | −4.531 | 1.00 | 249.60 |
| 6366 | CB | GLU | E | 33 | 16.663 | 79.497 | −3.101 | 1.00 | 249.49 |
| 6367 | CG | GLU | E | 33 | 17.893 | 80.382 | −2.971 | 1.00 | 249.49 |
| 6368 | CD | GLU | E | 33 | 17.724 | 81.732 | −3.659 | 1.00 | 249.49 |
| 6369 | OE1 | GLU | E | 33 | 16.677 | 82.386 | −3.446 | 1.00 | 249.49 |
| 6370 | OE2 | GLU | E | 33 | 18.641 | 82.137 | −4.409 | 1.00 | 249.49 |
| 6371 | C | GLU | E | 33 | 17.112 | 78.603 | −5.404 | 1.00 | 249.60 |
| 6372 | O | GLU | E | 33 | 17.820 | 79.197 | −6.225 | 1.00 | 249.60 |
| 6373 | N | VAL | E | 34 | 17.179 | 77.285 | −5.229 | 1.00 | 249.69 |
| 6374 | CA | VAL | E | 34 | 18.123 | 76.456 | −5.982 | 1.00 | 249.69 |
| 6375 | CB | VAL | E | 34 | 18.005 | 74.970 | −5.541 | 1.00 | 249.69 |
| 6376 | CG1 | VAL | E | 34 | 19.019 | 74.118 | −6.281 | 1.00 | 249.69 |
| 6377 | CG2 | VAL | E | 34 | 18.228 | 74.858 | −4.032 | 1.00 | 249.69 |
| 6378 | C | VAL | E | 34 | 18.014 | 76.540 | −7.511 | 1.00 | 249.69 |
| 6379 | O | VAL | E | 34 | 16.936 | 76.775 | −8.062 | 1.00 | 249.69 |
| 6380 | N | SER | E | 35 | 19.152 | 76.355 | −8.178 | 1.00 | 248.45 |
| 6381 | CA | SER | E | 35 | 19.231 | 76.387 | −9.638 | 1.00 | 248.45 |
| 6382 | CB | SER | E | 35 | 20.377 | 77.287 | −10.097 | 1.00 | 249.69 |
| 6383 | OG | SER | E | 35 | 21.627 | 76.682 | −9.830 | 1.00 | 249.69 |
| 6384 | C | SER | E | 35 | 19.485 | 74.969 | −10.140 | 1.00 | 248.45 |
| 6385 | O | SER | E | 35 | 19.572 | 74.732 | −11.350 | 1.00 | 248.45 |
| 6386 | N | SER | E | 36 | 19.622 | 74.036 | −9.196 | 1.00 | 249.69 |
| 6387 | CA | SER | E | 36 | 19.865 | 72.628 | −9.516 | 1.00 | 249.69 |
| 6388 | CB | SER | E | 36 | 20.966 | 72.043 | −8.614 | 1.00 | 244.12 |
| 6389 | OG | SER | E | 36 | 20.521 | 71.874 | −7.278 | 1.00 | 244.12 |
| 6390 | C | SER | E | 36 | 18.578 | 71.819 | −9.352 | 1.00 | 249.69 |
| 6391 | O | SER | E | 36 | 18.259 | 71.325 | −8.266 | 1.00 | 249.69 |
| 6392 | N | THR | E | 37 | 17.836 | 71.701 | −10.449 | 1.00 | 208.63 |
| 6393 | CA | THR | E | 37 | 16.589 | 70.958 | −10.462 | 1.00 | 208.63 |
| 6394 | CB | THR | E | 37 | 15.388 | 71.911 | −10.670 | 1.00 | 197.00 |
| 6395 | OG1 | THR | E | 37 | 15.343 | 72.880 | −9.604 | 1.00 | 197.00 |
| 6396 | CG2 | THR | E | 37 | 14.089 | 71.128 | −10.693 | 1.00 | 197.00 |
| 6397 | C | THR | E | 37 | 16.689 | 69.957 | −11.608 | 1.00 | 208.63 |
| 6398 | O | THR | E | 37 | 17.186 | 70.277 | −12.687 | 1.00 | 208.63 |
| 6399 | N | LYS | E | 38 | 16.236 | 68.739 | −11.366 | 1.00 | 223.46 |
| 6400 | CA | LYS | E | 38 | 16.309 | 67.706 | −12.384 | 1.00 | 223.46 |
| 6401 | CB | LYS | E | 38 | 16.899 | 66.425 | −11.765 | 1.00 | 231.11 |
| 6402 | CG | LYS | E | 38 | 18.295 | 66.620 | −11.158 | 1.00 | 231.11 |
| 6403 | CD | LYS | E | 38 | 18.852 | 65.344 | −10.528 | 1.00 | 231.11 |
| 6404 | CE | LYS | E | 38 | 20.261 | 65.567 | −9.976 | 1.00 | 231.11 |
| 6405 | NZ | LYS | E | 38 | 20.853 | 64.342 | −9.367 | 1.00 | 231.11 |
| 6406 | C | LYS | E | 38 | 14.947 | 67.420 | −13.018 | 1.00 | 223.46 |
| 6407 | O | LYS | E | 38 | 13.914 | 67.492 | −12.361 | 1.00 | 223.46 |
| 6408 | N | TRP | E | 39 | 14.951 | 67.117 | −14.308 | 1.00 | 249.08 |
| 6409 | CA | TRP | E | 39 | 13.721 | 66.798 | −15.022 | 1.00 | 249.08 |
| 6410 | CB | TRP | E | 39 | 13.373 | 67.909 | −16.006 | 1.00 | 173.34 |
| 6411 | CG | TRP | E | 39 | 12.996 | 69.216 | −15.384 | 1.00 | 173.34 |
| 6412 | CD2 | TRP | E | 39 | 11.899 | 69.476 | −14.483 | 1.00 | 173.34 |
| 6413 | CE2 | TRP | E | 39 | 11.857 | 70.869 | −14.267 | 1.00 | 173.34 |
| 6414 | CE3 | TRP | E | 39 | 10.957 | 68.673 | −13.840 | 1.00 | 173.34 |
| 6415 | CD1 | TRP | E | 39 | 13.550 | 70.422 | −15.660 | 1.00 | 173.34 |
| 6416 | NE1 | TRP | E | 39 | 12.871 | 71.424 | −14.998 | 1.00 | 173.34 |
| 6417 | CZ2 | TRP | E | 39 | 10.911 | 71.476 | −13.440 | 1.00 | 173.34 |
| 6418 | CZ3 | TRP | E | 39 | 10.011 | 69.281 | −13.014 | 1.00 | 173.34 |
| 6419 | CH2 | TRP | E | 39 | 9.995 | 70.668 | −12.828 | 1.00 | 173.34 |
| 6420 | C | TRP | E | 39 | 13.964 | 65.501 | −15.788 | 1.00 | 249.08 |
| 6421 | O | TRP | E | 39 | 14.993 | 65.363 | −16.450 | 1.00 | 249.08 |
| 6422 | N | PHE | E | 40 | 13.032 | 64.553 | −15.700 | 1.00 | 178.94 |
| 6423 | CA | PHE | E | 40 | 13.206 | 63.281 | −16.394 | 1.00 | 178.94 |
| 6424 | CB | PHE | E | 40 | 13.435 | 62.145 | −15.383 | 1.00 | 249.69 |
| 6425 | CG | PHE | E | 40 | 14.631 | 62.341 | −14.475 | 1.00 | 249.69 |
| 6426 | CD1 | PHE | E | 40 | 14.543 | 63.160 | −13.347 | 1.00 | 249.69 |
| 6427 | CD2 | PHE | E | 40 | 15.836 | 61.678 | −14.729 | 1.00 | 249.69 |
| 6428 | CE1 | PHE | E | 40 | 15.632 | 63.313 | −12.484 | 1.00 | 249.69 |
| 6429 | CE2 | PHE | E | 40 | 16.928 | 61.826 | −13.873 | 1.00 | 249.69 |
| 6430 | CZ | PHE | E | 40 | 16.824 | 62.645 | −12.748 | 1.00 | 249.69 |
| 6431 | C | PHE | E | 40 | 12.051 | 62.896 | −17.323 | 1.00 | 178.94 |
| 6432 | O | PHE | E | 40 | 11.245 | 62.027 | −16.988 | 1.00 | 178.94 |
| 6433 | N | HIS | E | 41 | 11.992 | 63.534 | −18.488 | 1.00 | 163.57 |
| 6434 | CA | HIS | E | 41 | 10.961 | 63.256 | −19.477 | 1.00 | 163.57 |
| 6435 | CB | HIS | E | 41 | 11.070 | 64.259 | −20.627 | 1.00 | 157.46 |
| 6436 | CG | HIS | E | 41 | 10.025 | 64.069 | −21.695 | 1.00 | 157.46 |
| 6437 | CD2 | HIS | E | 41 | 10.104 | 64.164 | −23.048 | 1.00 | 157.46 |
| 6438 | ND1 | HIS | E | 41 | 8.710 | 63.777 | −21.411 | 1.00 | 157.46 |
| 6439 | CE1 | HIS | E | 41 | 8.018 | 63.698 | −22.540 | 1.00 | 157.46 |
| 6440 | NE2 | HIS | E | 41 | 8.840 | 63.930 | −23.544 | 1.00 | 157.46 |
| 6441 | C | HIS | E | 41 | 11.067 | 61.816 | −20.031 | 1.00 | 163.57 |
| 6442 | O | HIS | E | 41 | 11.955 | 61.520 | −20.841 | 1.00 | 163.57 |
| 6443 | N | ASN | E | 42 | 10.149 | 60.937 | −19.611 | 1.00 | 166.06 |
| 6444 | CA | ASN | E | 42 | 10.139 | 59.529 | −20.031 | 1.00 | 166.06 |
| 6445 | CB | ASN | E | 42 | 10.165 | 59.393 | −21.564 | 1.00 | 227.72 |
| 6446 | CG | ASN | E | 42 | 8.800 | 59.655 | −22.205 | 1.00 | 227.72 |
| 6447 | OD1 | ASN | E | 42 | 8.150 | 60.654 | −21.900 | 1.00 | 227.72 |
| 6448 | ND2 | ASN | E | 42 | 8.370 | 58.767 | −23.101 | 1.00 | 227.72 |
| 6449 | C | ASN | E | 42 | 11.348 | 58.828 | −19.424 | 1.00 | 166.06 |
| 6450 | O | ASN | E | 42 | 11.820 | 57.822 | −19.950 | 1.00 | 166.06 |
| 6451 | N | GLY | E | 43 | 11.829 | 59.368 | −18.305 | 1.00 | 222.62 |
| 6452 | CA | GLY | E | 43 | 12.985 | 58.804 | −17.627 | 1.00 | 222.62 |
| 6453 | C | GLY | E | 43 | 14.272 | 59.493 | −18.049 | 1.00 | 222.62 |
| 6454 | O | GLY | E | 43 | 15.139 | 59.780 | −17.220 | 1.00 | 222.62 |
| 6455 | N | SER | E | 44 | 14.388 | 59.764 | −19.346 | 1.00 | 232.48 |
| 6456 | CA | SER | E | 44 | 15.560 | 60.424 | −19.919 | 1.00 | 232.48 |
| 6457 | CB | SER | E | 44 | 15.391 | 60.572 | −21.435 | 1.00 | 196.92 |
| 6458 | OG | SER | E | 44 | 15.207 | 59.322 | −22.064 | 1.00 | 196.92 |
| 6459 | C | SER | E | 44 | 15.788 | 61.806 | −19.322 | 1.00 | 232.48 |
| 6460 | O | SER | E | 44 | 14.908 | 62.661 | −19.386 | 1.00 | 232.48 |
| 6461 | N | LEU | E | 45 | 16.970 | 62.033 | −18.759 | 1.00 | 247.61 |
| 6462 | CA | LEU | E | 45 | 17.273 | 63.331 | −18.173 | 1.00 | 247.61 |
| 6463 | CB | LEU | E | 45 | 18.722 | 63.380 | −17.682 | 1.00 | 238.67 |
| 6464 | CG | LEU | E | 45 | 19.128 | 64.690 | −16.996 | 1.00 | 238.67 |
| 6465 | CD1 | LEU | E | 45 | 18.176 | 64.989 | −15.849 | 1.00 | 238.67 |
| 6466 | CD2 | LEU | E | 45 | 20.552 | 64.584 | −16.486 | 1.00 | 238.67 |
| 6467 | C | LEU | E | 45 | 17.030 | 64.445 | −19.194 | 1.00 | 247.61 |
| 6468 | O | LEU | E | 45 | 17.195 | 64.244 | −20.401 | 1.00 | 247.61 |
| 6469 | N | SER | E | 46 | 16.630 | 65.616 | −18.700 | 1.00 | 233.41 |
| 6470 | CA | SER | E | 46 | 16.339 | 66.768 | −19.550 | 1.00 | 233.41 |
| 6471 | CB | SER | E | 46 | 15.009 | 67.411 | −19.131 | 1.00 | 241.98 |
| 6472 | OG | SER | E | 46 | 14.644 | 68.461 | −20.012 | 1.00 | 241.98 |
| 6473 | C | SER | E | 46 | 17.450 | 67.806 | −19.498 | 1.00 | 233.41 |
| 6474 | O | SER | E | 46 | 18.358 | 67.721 | −18.670 | 1.00 | 233.41 |
| 6475 | N | GLU | E | 47 | 17.353 | 68.798 | −20.378 | 1.00 | 249.69 |
| 6476 | CA | GLU | E | 47 | 18.353 | 69.858 | −20.479 | 1.00 | 249.69 |
| 6477 | CB | GLU | E | 47 | 18.508 | 70.268 | −21.943 | 1.00 | 249.69 |
| 6478 | CG | GLU | E | 47 | 18.990 | 69.136 | −22.837 | 1.00 | 249.69 |
| 6479 | CD | GLU | E | 47 | 19.114 | 69.555 | −24.287 | 1.00 | 249.69 |
| 6480 | OE1 | GLU | E | 47 | 18.075 | 69.901 | −24.896 | 1.00 | 249.69 |
| 6481 | OE2 | GLU | E | 47 | 20.247 | 69.540 | −24.819 | 1.00 | 249.69 |
| 6482 | C | GLU | E | 47 | 18.118 | 71.111 | −19.629 | 1.00 | 249.69 |
| 6483 | O | GLU | E | 47 | 19.014 | 71.949 | −19.500 | 1.00 | 249.69 |
| 6484 | N | GLU | E | 48 | 16.925 | 71.256 | −19.062 | 1.00 | 197.12 |
| 6485 | CA | GLU | E | 48 | 16.652 | 72.417 | −18.231 | 1.00 | 197.12 |
| 6486 | CB | GLU | E | 48 | 15.153 | 72.734 | −18.213 | 1.00 | 231.64 |
| 6487 | CG | GLU | E | 48 | 14.768 | 73.878 | −17.276 | 1.00 | 231.64 |
| 6488 | CD | GLU | E | 48 | 15.355 | 75.216 | −17.688 | 1.00 | 231.64 |
| 6489 | OE1 | GLU | E | 48 | 14.884 | 75.786 | −18.695 | 1.00 | 231.64 |
| 6490 | OE2 | GLU | E | 48 | 16.286 | 75.698 | −17.004 | 1.00 | 231.64 |
| 6491 | C | GLU | E | 48 | 17.147 | 72.146 | −16.813 | 1.00 | 197.12 |
| 6492 | O | GLU | E | 48 | 17.288 | 70.990 | −16.397 | 1.00 | 197.12 |
| 6493 | N | THR | E | 49 | 17.420 | 73.221 | −16.080 | 1.00 | 219.19 |
| 6494 | CA | THR | E | 49 | 17.901 | 73.124 | −14.707 | 1.00 | 219.19 |
| 6495 | CB | THR | E | 49 | 19.370 | 73.557 | −14.610 | 1.00 | 249.63 |
| 6496 | OG1 | THR | E | 49 | 19.517 | 74.871 | −15.170 | 1.00 | 249.63 |
| 6497 | CG2 | THR | E | 49 | 20.263 | 72.574 | −15.363 | 1.00 | 249.63 |
| 6498 | C | THR | E | 49 | 17.060 | 74.007 | −13.794 | 1.00 | 219.19 |
| 6499 | O | THR | E | 49 | 16.949 | 73.749 | −12.597 | 1.00 | 219.19 |
| 6500 | N | ASN | E | 50 | 16.475 | 75.055 | −14.364 | 1.00 | 248.24 |
| 6501 | CA | ASN | E | 50 | 15.625 | 75.963 | −13.603 | 1.00 | 248.24 |
| 6502 | CB | ASN | E | 50 | 15.070 | 77.051 | −14.530 | 1.00 | 249.69 |
| 6503 | CG | ASN | E | 50 | 14.389 | 78.172 | −13.770 | 1.00 | 249.69 |
| 6504 | OD1 | ASN | E | 50 | 14.025 | 78.004 | −12.606 | 1.00 | 249.69 |
| 6505 | ND2 | ASN | E | 50 | 14.201 | 79.314 | −14.428 | 1.00 | 249.69 |
| 6506 | C | ASN | E | 50 | 14.479 | 75.129 | −13.024 | 1.00 | 248.24 |
| 6507 | O | ASN | E | 50 | 14.117 | 74.104 | −13.598 | 1.00 | 248.24 |
| 6508 | N | SER | E | 51 | 13.906 | 75.553 | −11.900 | 1.00 | 208.41 |
| 6509 | CA | SER | E | 51 | 12.811 | 74.797 | −11.296 | 1.00 | 208.41 |
| 6510 | CB | SER | E | 51 | 12.509 | 75.325 | −9.894 | 1.00 | 181.70 |
| 6511 | OG | SER | E | 51 | 11.848 | 76.577 | −9.948 | 1.00 | 181.70 |
| 6512 | C | SER | E | 51 | 11.524 | 74.814 | −12.142 | 1.00 | 208.41 |
| 6513 | O | SER | E | 51 | 10.625 | 73.998 | −11.929 | 1.00 | 208.41 |
| 6514 | N | SER | E | 52 | 11.434 | 75.735 | −13.100 | 1.00 | 201.05 |
| 6515 | CA | SER | E | 52 | 10.254 | 75.830 | −13.962 | 1.00 | 201.05 |
| 6516 | CB | SER | E | 52 | 9.717 | 77.259 | −13.995 | 1.00 | 181.26 |
| 6517 | OG | SER | E | 52 | 9.309 | 77.679 | −12.707 | 1.00 | 181.26 |
| 6518 | C | SER | E | 52 | 10.551 | 75.390 | −15.385 | 1.00 | 201.05 |
| 6519 | O | SER | E | 52 | 11.180 | 76.117 | −16.157 | 1.00 | 201.05 |
| 6520 | N | LEU | E | 53 | 10.088 | 74.194 | −15.724 | 1.00 | 168.60 |
| 6521 | CA | LEU | E | 53 | 10.287 | 73.643 | −17.058 | 1.00 | 168.60 |
| 6522 | CB | LEU | E | 53 | 10.514 | 72.132 | −16.970 | 1.00 | 122.87 |
| 6523 | CG | LEU | E | 53 | 10.304 | 71.282 | −18.231 | 1.00 | 122.87 |
| 6524 | CD1 | LEU | E | 53 | 10.969 | 71.906 | −19.463 | 1.00 | 122.87 |
| 6525 | CD2 | LEU | E | 53 | 10.854 | 69.900 | −17.957 | 1.00 | 122.87 |
| 6526 | C | LEU | E | 53 | 9.077 | 73.940 | −17.940 | 1.00 | 168.60 |
| 6527 | O | LEU | E | 53 | 8.020 | 73.327 | −17.805 | 1.00 | 168.60 |
| 6528 | N | ASN | E | 54 | 9.235 | 74.892 | −18.848 | 1.00 | 150.56 |
| 6529 | CA | ASN | E | 54 | 8.149 | 75.251 | −19.729 | 1.00 | 150.56 |
| 6530 | CB | ASN | E | 54 | 8.319 | 76.683 | −20.233 | 1.00 | 229.83 |
| 6531 | CG | ASN | E | 54 | 8.080 | 77.699 | −19.151 | 1.00 | 229.83 |
| 6532 | OD1 | ASN | E | 54 | 7.052 | 77.669 | −18.479 | 1.00 | 229.83 |
| 6533 | ND2 | ASN | E | 54 | 9.029 | 78.606 | −18.972 | 1.00 | 229.83 |
| 6534 | C | ASN | E | 54 | 8.032 | 74.311 | −20.905 | 1.00 | 150.56 |
| 6535 | O | ASN | E | 54 | 8.977 | 73.598 | −21.260 | 1.00 | 150.56 |
| 6536 | N | ILE | E | 55 | 6.840 | 74.322 | −21.495 | 1.00 | 211.09 |
| 6537 | CA | ILE | E | 55 | 6.499 | 73.504 | −22.654 | 1.00 | 211.09 |
| 6538 | CB | ILE | E | 55 | 5.596 | 72.306 | −22.246 | 1.00 | 170.97 |
| 6539 | CG2 | ILE | E | 55 | 4.804 | 71.809 | −23.445 | 1.00 | 170.97 |
| 6540 | CG1 | ILE | E | 55 | 6.460 | 71.189 | −21.638 | 1.00 | 170.97 |
| 6541 | CD1 | ILE | E | 55 | 5.686 | 69.960 | −21.201 | 1.00 | 170.97 |
| 6542 | C | ILE | E | 55 | 5.745 | 74.400 | −23.628 | 1.00 | 211.09 |
| 6543 | O | ILE | E | 55 | 4.659 | 74.893 | −23.314 | 1.00 | 211.09 |
| 6544 | N | VAL | E | 56 | 6.329 | 74.624 | −24.799 | 1.00 | 155.17 |
| 6545 | CA | VAL | E | 56 | 5.684 | 75.466 | −25.778 | 1.00 | 155.17 |
| 6546 | CB | VAL | E | 56 | 6.705 | 76.318 | −26.516 | 1.00 | 247.89 |
| 6547 | CG1 | VAL | E | 56 | 6.008 | 77.514 | −27.154 | 1.00 | 247.89 |
| 6548 | CG2 | VAL | E | 56 | 7.780 | 76.778 | −25.550 | 1.00 | 247.89 |
| 6549 | C | VAL | E | 56 | 4.917 | 74.606 | −26.770 | 1.00 | 155.17 |
| 6550 | O | VAL | E | 56 | 4.585 | 73.468 | −26.458 | 1.00 | 155.17 |
| 6551 | N | ASN | E | 57 | 4.639 | 75.144 | −27.959 | 1.00 | 169.22 |
| 6552 | CA | ASN | E | 57 | 3.886 | 74.432 | −28.996 | 1.00 | 169.22 |
| 6553 | CB | ASN | E | 57 | 4.365 | 74.837 | −30.389 | 1.00 | 237.27 |
| 6554 | CG | ASN | E | 57 | 3.979 | 76.263 | −30.731 | 1.00 | 237.27 |
| 6555 | OD1 | ASN | E | 57 | 2.817 | 76.650 | −30.605 | 1.00 | 237.27 |
| 6556 | ND2 | ASN | E | 57 | 4.953 | 77.055 | −31.165 | 1.00 | 237.27 |
| 6557 | C | ASN | E | 57 | 3.941 | 72.925 | −28.825 | 1.00 | 169.22 |
| 6558 | O | ASN | E | 57 | 4.860 | 72.255 | −29.300 | 1.00 | 169.22 |
| 6559 | N | ALA | E | 58 | 2.923 | 72.424 | −28.128 | 1.00 | 152.74 |
| 6560 | CA | ALA | E | 58 | 2.746 | 71.015 | −27.787 | 1.00 | 152.74 |
| 6561 | CB | ALA | E | 58 | 1.438 | 70.847 | −27.040 | 1.00 | 133.90 |
| 6562 | C | ALA | E | 58 | 2.812 | 70.012 | −28.932 | 1.00 | 152.74 |
| 6563 | O | ALA | E | 58 | 1.888 | 69.915 | −29.751 | 1.00 | 152.74 |
| 6564 | N | LYS | E | 59 | 3.909 | 69.258 | −28.966 | 1.00 | 179.74 |
| 6565 | CA | LYS | E | 59 | 4.128 | 68.226 | −29.976 | 1.00 | 179.74 |
| 6566 | CB | LYS | E | 59 | 5.572 | 68.264 | −30.469 | 1.00 | 249.69 |
| 6567 | CG | LYS | E | 59 | 5.967 | 69.596 | −31.093 | 1.00 | 249.69 |
| 6568 | CD | LYS | E | 59 | 7.446 | 69.638 | −31.470 | 1.00 | 249.69 |
| 6569 | CE | LYS | E | 59 | 7.825 | 71.010 | −32.032 | 1.00 | 249.69 |
| 6570 | NZ | LYS | E | 59 | 9.265 | 71.091 | −32.425 | 1.00 | 249.69 |
| 6571 | C | LYS | E | 59 | 3.853 | 66.896 | −29.300 | 1.00 | 179.74 |
| 6572 | O | LYS | E | 59 | 4.242 | 66.677 | −28.156 | 1.00 | 179.74 |
| 6573 | N | PHE | E | 60 | 3.175 | 66.009 | −30.009 | 1.00 | 187.69 |
| 6574 | CA | PHE | E | 60 | 2.825 | 64.706 | −29.466 | 1.00 | 187.69 |
| 6575 | CB | PHE | E | 60 | 2.441 | 63.771 | −30.613 | 1.00 | 249.39 |
| 6576 | CG | PHE | E | 60 | 1.209 | 64.201 | −31.352 | 1.00 | 249.39 |
| 6577 | CD1 | PHE | E | 60 | 1.035 | 63.865 | −32.689 | 1.00 | 249.39 |
| 6578 | CD2 | PHE | E | 60 | 0.214 | 64.930 | −30.707 | 1.00 | 249.39 |
| 6579 | CE1 | PHE | E | 60 | −0.111 | 64.248 | −33.377 | 1.00 | 249.39 |
| 6580 | CE2 | PHE | E | 60 | −0.939 | 65.319 | −31.386 | 1.00 | 249.39 |
| 6581 | CZ | PHE | E | 60 | −1.101 | 64.976 | −32.724 | 1.00 | 249.39 |
| 6582 | C | PHE | E | 60 | 3.927 | 64.073 | −28.626 | 1.00 | 187.69 |
| 6583 | O | PHE | E | 60 | 3.642 | 63.338 | −27.683 | 1.00 | 187.69 |
| 6584 | N | GLU | E | 61 | 5.181 | 64.364 | −28.965 | 1.00 | 196.09 |
| 6585 | CA | GLU | E | 61 | 6.335 | 63.808 | −28.255 | 1.00 | 196.09 |
| 6586 | CB | GLU | E | 61 | 7.623 | 64.147 | −29.010 | 1.00 | 249.51 |
| 6587 | CG | GLU | E | 61 | 7.682 | 63.618 | −30.447 | 1.00 | 249.51 |
| 6588 | CD | GLU | E | 61 | 6.550 | 64.132 | −31.336 | 1.00 | 249.51 |
| 6589 | OE1 | GLU | E | 61 | 6.341 | 65.363 | −31.403 | 1.00 | 249.51 |
| 6590 | OE2 | GLU | E | 61 | 5.872 | 63.302 | −31.976 | 1.00 | 249.51 |
| 6591 | C | GLU | E | 61 | 6.433 | 64.313 | −26.820 | 1.00 | 196.09 |
| 6592 | O | GLU | E | 61 | 7.042 | 63.668 | −25.965 | 1.00 | 196.09 |
| 6593 | N | ASP | E | 62 | 5.831 | 65.470 | −26.566 | 1.00 | 216.24 |
| 6594 | CA | ASP | E | 62 | 5.842 | 66.063 | −25.235 | 1.00 | 216.24 |
| 6595 | CB | ASP | E | 62 | 5.333 | 67.500 | −25.285 | 1.00 | 214.73 |
| 6596 | CG | ASP | E | 62 | 6.047 | 68.327 | −26.319 | 1.00 | 214.73 |
| 6597 | OD1 | ASP | E | 62 | 7.249 | 68.076 | −26.535 | 1.00 | 214.73 |
| 6598 | OD2 | ASP | E | 62 | 5.416 | 69.232 | −26.906 | 1.00 | 214.73 |
| 6599 | C | ASP | E | 62 | 4.971 | 65.264 | −24.279 | 1.00 | 216.24 |
| 6600 | O | ASP | E | 62 | 5.152 | 65.325 | −23.063 | 1.00 | 216.24 |
| 6601 | N | SER | E | 63 | 4.009 | 64.532 | −24.831 | 1.00 | 123.78 |
| 6602 | CA | SER | E | 63 | 3.127 | 63.707 | −24.015 | 1.00 | 123.78 |
| 6603 | CB | SER | E | 63 | 2.085 | 62.988 | −24.899 | 1.00 | 115.99 |
| 6604 | OG | SER | E | 63 | 1.319 | 63.897 | −25.674 | 1.00 | 115.99 |
| 6605 | C | SER | E | 63 | 4.011 | 62.669 | −23.301 | 1.00 | 123.78 |
| 6606 | O | SER | E | 63 | 4.783 | 61.957 | −23.934 | 1.00 | 123.78 |
| 6607 | N | GLY | E | 64 | 3.908 | 62.584 | −21.986 | 1.00 | 143.97 |
| 6608 | CA | GLY | E | 64 | 4.723 | 61.615 | −21.294 | 1.00 | 143.97 |
| 6609 | C | GLY | E | 64 | 4.739 | 61.721 | −19.786 | 1.00 | 143.97 |
| 6610 | O | GLY | E | 64 | 3.910 | 62.406 | −19.184 | 1.00 | 143.97 |
| 6611 | N | GLU | E | 65 | 5.707 | 61.027 | −19.190 | 1.00 | 147.25 |
| 6612 | CA | GLU | E | 65 | 5.904 | 60.964 | −17.743 | 1.00 | 147.25 |
| 6613 | CB | GLU | E | 65 | 6.138 | 59.507 | −17.358 | 1.00 | 197.50 |
| 6614 | CG | GLU | E | 65 | 6.548 | 59.268 | −15.932 | 1.00 | 197.50 |
| 6615 | CD | GLU | E | 65 | 7.152 | 57.894 | −15.759 | 1.00 | 197.50 |
| 6616 | OE1 | GLU | E | 65 | 8.198 | 57.630 | −16.379 | 1.00 | 197.50 |
| 6617 | OE2 | GLU | E | 65 | 6.587 | 57.076 | −15.015 | 1.00 | 197.50 |
| 6618 | C | GLU | E | 65 | 7.097 | 61.824 | −17.309 | 1.00 | 147.25 |
| 6619 | O | GLU | E | 65 | 8.215 | 61.604 | −17.766 | 1.00 | 147.25 |
| 6620 | N | TYR | E | 66 | 6.859 | 62.792 | −16.423 | 1.00 | 205.23 |
| 6621 | CA | TYR | E | 66 | 7.924 | 63.682 | −15.948 | 1.00 | 205.23 |
| 6622 | CB | TYR | E | 66 | 7.595 | 65.141 | −16.266 | 1.00 | 153.79 |
| 6623 | CG | TYR | E | 66 | 7.502 | 65.522 | −17.726 | 1.00 | 153.79 |
| 6624 | CD1 | TYR | E | 66 | 6.391 | 65.194 | −18.490 | 1.00 | 153.79 |
| 6625 | CE1 | TYR | E | 66 | 6.265 | 65.628 | −19.806 | 1.00 | 153.79 |
| 6626 | CD2 | TYR | E | 66 | 8.491 | 66.286 | −18.316 | 1.00 | 153.79 |
| 6627 | CE2 | TYR | E | 66 | 8.377 | 66.729 | −19.628 | 1.00 | 153.79 |
| 6628 | CZ | TYR | E | 66 | 7.265 | 66.403 | −20.368 | 1.00 | 153.79 |
| 6629 | OH | TYR | E | 66 | 7.159 | 66.870 | −21.663 | 1.00 | 153.79 |
| 6630 | C | TYR | E | 66 | 8.179 | 63.597 | −14.442 | 1.00 | 205.23 |
| 6631 | O | TYR | E | 66 | 7.402 | 62.979 | −13.710 | 1.00 | 205.23 |
| 6632 | N | LYS | E | 67 | 9.261 | 64.248 | −13.997 | 1.00 | 187.13 |
| 6633 | CA | LYS | E | 67 | 9.646 | 64.287 | −12.580 | 1.00 | 187.13 |
| 6634 | CB | LYS | E | 67 | 9.961 | 62.882 | −12.071 | 1.00 | 169.14 |
| 6635 | CG | LYS | E | 67 | 10.969 | 62.131 | −12.908 | 1.00 | 169.14 |
| 6636 | CD | LYS | E | 67 | 11.160 | 60.730 | −12.365 | 1.00 | 169.14 |
| 6637 | CE | LYS | E | 67 | 11.800 | 59.810 | −13.402 | 1.00 | 169.14 |
| 6638 | NZ | LYS | E | 67 | 12.045 | 58.414 | −12.897 | 1.00 | 169.14 |
| 6639 | C | LYS | E | 67 | 10.842 | 65.185 | −12.276 | 1.00 | 187.13 |
| 6640 | O | LYS | E | 67 | 11.685 | 65.433 | −13.141 | 1.00 | 187.13 |
| 6641 | N | CYS | E | 68 | 10.902 | 65.666 | −11.032 | 1.00 | 161.02 |
| 6642 | CA | CYS | E | 68 | 12.004 | 66.515 | −10.582 | 1.00 | 161.02 |
| 6643 | C | CYS | E | 68 | 12.590 | 66.008 | −9.267 | 1.00 | 161.02 |
| 6644 | O | CYS | E | 68 | 11.900 | 65.396 | −8.456 | 1.00 | 161.02 |
| 6645 | CB | CYS | E | 68 | 11.567 | 67.993 | −10.447 | 1.00 | 132.43 |
| 6646 | SG | CYS | E | 68 | 10.402 | 68.415 | −9.105 | 1.00 | 132.43 |
| 6647 | N | GLN | E | 69 | 13.880 | 66.274 | −9.084 | 1.00 | 233.39 |
| 6648 | CA | GLN | E | 69 | 14.623 | 65.865 | −7.903 | 1.00 | 233.39 |
| 6649 | CB | GLN | E | 69 | 15.252 | 64.494 | −8.156 | 1.00 | 249.52 |
| 6650 | CG | GLN | E | 69 | 16.420 | 64.156 | −7.253 | 1.00 | 249.52 |
| 6651 | CD | GLN | E | 69 | 17.095 | 62.850 | −7.643 | 1.00 | 249.52 |
| 6652 | OE1 | GLN | E | 69 | 17.462 | 62.650 | −8.803 | 1.00 | 249.52 |
| 6653 | NE2 | GLN | E | 69 | 17.266 | 61.957 | −6.673 | 1.00 | 249.52 |
| 6654 | C | GLN | E | 69 | 15.709 | 66.900 | −7.632 | 1.00 | 233.39 |
| 6655 | O | GLN | E | 69 | 16.242 | 67.505 | −8.563 | 1.00 | 233.39 |
| 6656 | N | HIS | E | 70 | 16.037 | 67.108 | −6.363 | 1.00 | 249.69 |
| 6657 | CA | HIS | E | 70 | 17.076 | 68.070 | −6.015 | 1.00 | 249.69 |
| 6658 | CB | HIS | E | 70 | 16.657 | 68.885 | −4.790 | 1.00 | 243.01 |
| 6659 | CG | HIS | E | 70 | 15.547 | 69.852 | −5.068 | 1.00 | 243.01 |
| 6660 | CD2 | HIS | E | 70 | 14.356 | 70.039 | −4.454 | 1.00 | 243.01 |
| 6661 | ND1 | HIS | E | 70 | 15.605 | 70.774 | −6.089 | 1.00 | 243.01 |
| 6662 | CE1 | HIS | E | 70 | 14.495 | 71.493 | −6.096 | 1.00 | 243.01 |
| 6663 | NE2 | HIS | E | 70 | 13.721 | 71.068 | −5.114 | 1.00 | 243.01 |
| 6664 | C | HIS | E | 70 | 18.414 | 67.388 | −5.761 | 1.00 | 249.69 |
| 6665 | O | HIS | E | 70 | 18.575 | 66.198 | −6.034 | 1.00 | 249.69 |
| 6666 | N | GLN | E | 71 | 19.370 | 68.148 | −5.238 | 1.00 | 231.62 |
| 6667 | CA | GLN | E | 71 | 20.708 | 67.634 | −4.952 | 1.00 | 231.62 |
| 6668 | CB | GLN | E | 71 | 21.554 | 68.756 | −4.329 | 1.00 | 249.69 |
| 6669 | CG | GLN | E | 71 | 23.080 | 68.559 | −4.380 | 1.00 | 249.69 |
| 6670 | CD | GLN | E | 71 | 23.634 | 68.488 | −5.803 | 1.00 | 249.69 |
| 6671 | OE1 | GLN | E | 71 | 23.304 | 69.316 | −6.663 | 1.00 | 249.69 |
| 6672 | NE2 | GLN | E | 71 | 24.495 | 67.503 | −6.049 | 1.00 | 249.69 |
| 6673 | C | GLN | E | 71 | 20.677 | 66.413 | −4.022 | 1.00 | 231.62 |
| 6674 | O | GLN | E | 71 | 21.311 | 65.389 | −4.298 | 1.00 | 231.62 |
| 6675 | N | GLN | E | 72 | 19.928 | 66.528 | −2.928 | 1.00 | 249.69 |
| 6676 | CA | GLN | E | 72 | 19.819 | 65.448 | −1.951 | 1.00 | 249.69 |
| 6677 | CB | GLN | E | 72 | 20.601 | 65.834 | −0.689 | 1.00 | 249.69 |
| 6678 | CG | GLN | E | 72 | 20.626 | 64.791 | 0.419 | 1.00 | 249.69 |
| 6679 | CD | GLN | E | 72 | 21.284 | 65.315 | 1.687 | 1.00 | 249.69 |
| 6680 | OE1 | GLN | E | 72 | 22.439 | 65.746 | 1.664 | 1.00 | 249.69 |
| 6681 | NE2 | GLN | E | 72 | 20.549 | 65.281 | 2.799 | 1.00 | 249.69 |
| 6682 | C | GLN | E | 72 | 18.349 | 65.170 | −1.607 | 1.00 | 249.69 |
| 6683 | O | GLN | E | 72 | 17.948 | 65.208 | −0.444 | 1.00 | 249.69 |
| 6684 | N | VAL | E | 73 | 17.544 | 64.899 | −2.627 | 1.00 | 249.30 |
| 6685 | CA | VAL | E | 73 | 16.128 | 64.614 | −2.427 | 1.00 | 249.30 |
| 6686 | CB | VAL | E | 73 | 15.243 | 65.860 | −2.707 | 1.00 | 249.69 |
| 6687 | CG1 | VAL | E | 73 | 13.838 | 65.614 | −2.192 | 1.00 | 249.69 |
| 6688 | CG2 | VAL | E | 73 | 15.843 | 67.101 | −2.058 | 1.00 | 249.69 |
| 6689 | C | VAL | E | 73 | 15.709 | 63.512 | −3.386 | 1.00 | 249.30 |
| 6690 | O | VAL | E | 73 | 16.183 | 63.451 | −4.516 | 1.00 | 249.30 |
| 6691 | N | ASN | E | 74 | 14.817 | 62.642 | −2.937 | 1.00 | 249.46 |
| 6692 | CA | ASN | E | 74 | 14.345 | 61.555 | −3.783 | 1.00 | 249.46 |
| 6693 | CB | ASN | E | 74 | 13.714 | 60.460 | −2.912 | 1.00 | 249.69 |
| 6694 | CG | ASN | E | 74 | 14.665 | 59.960 | −1.825 | 1.00 | 249.69 |
| 6695 | OD1 | ASN | E | 74 | 15.859 | 59.769 | −2.080 | 1.00 | 249.69 |
| 6696 | ND2 | ASN | E | 74 | 14.139 | 59.737 | −0.621 | 1.00 | 249.69 |
| 6697 | C | ASN | E | 74 | 13.346 | 62.081 | −4.826 | 1.00 | 249.46 |
| 6698 | O | ASN | E | 74 | 12.387 | 62.774 | −4.485 | 1.00 | 249.46 |
| 6699 | N | GLU | E | 75 | 13.587 | 61.755 | −6.094 | 1.00 | 249.69 |
| 6700 | CA | GLU | E | 75 | 12.740 | 62.194 | −7.209 | 1.00 | 249.69 |
| 6701 | CB | GLU | E | 75 | 13.082 | 61.386 | −8.468 | 1.00 | 249.69 |
| 6702 | CG | GLU | E | 75 | 13.431 | 59.924 | −8.199 | 1.00 | 249.69 |
| 6703 | CD | GLU | E | 75 | 14.002 | 59.221 | −9.425 | 1.00 | 249.69 |
| 6704 | OE1 | GLU | E | 75 | 14.946 | 59.768 | −10.042 | 1.00 | 249.69 |
| 6705 | OE2 | GLU | E | 75 | 13.515 | 58.118 | −9.768 | 1.00 | 249.69 |
| 6706 | C | GLU | E | 75 | 11.233 | 62.147 | −6.941 | 1.00 | 249.69 |
| 6707 | O | GLU | E | 75 | 10.724 | 61.213 | −6.320 | 1.00 | 249.69 |
| 6708 | N | SER | E | 76 | 10.531 | 63.168 | −7.428 | 1.00 | 190.25 |
| 6709 | CA | SER | E | 76 | 9.083 | 63.302 | −7.244 | 1.00 | 190.25 |
| 6710 | CB | SER | E | 76 | 8.613 | 64.671 | −7.743 | 1.00 | 199.25 |
| 6711 | OG | SER | E | 76 | 8.614 | 64.717 | −9.163 | 1.00 | 199.25 |
| 6712 | C | SER | E | 76 | 8.265 | 62.236 | −7.954 | 1.00 | 190.25 |
| 6713 | O | SER | E | 76 | 8.728 | 61.612 | −8.907 | 1.00 | 190.25 |
| 6714 | N | GLU | E | 77 | 7.038 | 62.044 | −7.481 | 1.00 | 235.50 |
| 6715 | CA | GLU | E | 77 | 6.135 | 61.072 | −8.079 | 1.00 | 235.50 |
| 6716 | CB | GLU | E | 77 | 4.839 | 60.973 | −7.269 | 1.00 | 249.14 |
| 6717 | CG | GLU | E | 77 | 5.029 | 60.405 | −5.870 | 1.00 | 249.14 |
| 6718 | CD | GLU | E | 77 | 5.532 | 58.966 | −5.879 | 1.00 | 249.14 |
| 6719 | OE1 | GLU | E | 77 | 5.869 | 58.450 | −6.966 | 1.00 | 249.14 |
| 6720 | OE2 | GLU | E | 77 | 5.594 | 58.349 | −4.795 | 1.00 | 249.14 |
| 6721 | C | GLU | E | 77 | 5.826 | 61.543 | −9.488 | 1.00 | 235.50 |
| 6722 | O | GLU | E | 77 | 5.181 | 62.569 | −9.672 | 1.00 | 235.50 |
| 6723 | N | PRO | E | 78 | 6.280 | 60.791 | −10.505 | 1.00 | 173.10 |
| 6724 | CD | PRO | E | 78 | 6.805 | 59.420 | −10.383 | 1.00 | 109.00 |
| 6725 | CA | PRO | E | 78 | 6.055 | 61.144 | −11.915 | 1.00 | 173.10 |
| 6726 | CB | PRO | E | 78 | 6.320 | 59.831 | −12.652 | 1.00 | 109.00 |
| 6727 | CG | PRO | E | 78 | 7.324 | 59.145 | −11.779 | 1.00 | 109.00 |
| 6728 | C | PRO | E | 78 | 4.648 | 61.657 | −12.182 | 1.00 | 173.10 |
| 6729 | O | PRO | E | 78 | 3.721 | 61.375 | −11.410 | 1.00 | 173.10 |
| 6730 | N | VAL | E | 79 | 4.490 | 62.430 | −13.253 | 1.00 | 170.14 |
| 6731 | CA | VAL | E | 79 | 3.168 | 62.929 | −13.628 | 1.00 | 170.14 |
| 6732 | CB | VAL | E | 79 | 2.986 | 64.448 | −13.378 | 1.00 | 117.67 |
| 6733 | CG1 | VAL | E | 79 | 1.708 | 64.936 | −14.057 | 1.00 | 117.67 |
| 6734 | CG2 | VAL | E | 79 | 2.879 | 64.719 | −11.889 | 1.00 | 117.67 |
| 6735 | C | VAL | E | 79 | 3.081 | 62.669 | −15.111 | 1.00 | 170.14 |
| 6736 | O | VAL | E | 79 | 4.093 | 62.798 | −15.803 | 1.00 | 170.14 |
| 6737 | N | TYR | E | 80 | 1.903 | 62.292 | −15.605 | 1.00 | 121.68 |
| 6738 | CA | TYR | E | 80 | 1.782 | 62.023 | −17.028 | 1.00 | 121.68 |
| 6739 | CB | TYR | E | 80 | 1.072 | 60.700 | −17.272 | 1.00 | 162.42 |
| 6740 | CG | TYR | E | 80 | 1.291 | 60.179 | −18.675 | 1.00 | 162.42 |
| 6741 | CD1 | TYR | E | 80 | 2.439 | 59.447 | −18.995 | 1.00 | 162.42 |
| 6742 | CE1 | TYR | E | 80 | 2.661 | 58.981 | −20.301 | 1.00 | 162.42 |
| 6743 | CD2 | TYR | E | 80 | 0.370 | 60.437 | −19.696 | 1.00 | 162.42 |
| 6744 | CE2 | TYR | E | 80 | 0.588 | 59.980 | −21.002 | 1.00 | 162.42 |
| 6745 | CZ | TYR | E | 80 | 1.732 | 59.256 | −21.290 | 1.00 | 162.42 |
| 6746 | OH | TYR | E | 80 | 1.960 | 58.803 | −22.557 | 1.00 | 162.42 |
| 6747 | C | TYR | E | 80 | 1.040 | 63.132 | −17.752 | 1.00 | 121.68 |
| 6748 | O | TYR | E | 80 | 0.022 | 63.642 | −17.277 | 1.00 | 121.68 |
| 6749 | N | LEU | E | 81 | 1.562 | 63.511 | −18.906 | 1.00 | 108.83 |
| 6750 | CA | LEU | E | 81 | 0.943 | 64.545 | −19.706 | 1.00 | 108.83 |
| 6751 | CB | LEU | E | 81 | 1.930 | 65.677 | −19.978 | 1.00 | 110.26 |
| 6752 | CG | LEU | E | 81 | 1.380 | 66.724 | −20.946 | 1.00 | 110.26 |
| 6753 | CD1 | LEU | E | 81 | 0.173 | 67.390 | −20.296 | 1.00 | 110.26 |
| 6754 | CD2 | LEU | E | 81 | 2.432 | 67.748 | −21.292 | 1.00 | 110.26 |
| 6755 | C | LEU | E | 81 | 0.514 | 63.944 | −21.028 | 1.00 | 108.83 |
| 6756 | O | LEU | E | 81 | 1.308 | 63.312 | −21.701 | 1.00 | 108.83 |
| 6757 | N | GLU | E | 82 | −0.740 | 64.132 | −21.407 | 1.00 | 99.21 |
| 6758 | CA | GLU | E | 82 | −1.194 | 63.601 | −22.679 | 1.00 | 99.21 |
| 6759 | CB | GLU | E | 82 | −2.359 | 62.625 | −22.473 | 1.00 | 224.57 |
| 6760 | CG | GLU | E | 82 | −2.479 | 61.592 | −23.590 | 1.00 | 224.57 |
| 6761 | CD | GLU | E | 82 | −3.618 | 60.615 | −23.373 | 1.00 | 224.57 |
| 6762 | OE1 | GLU | E | 82 | −3.903 | 60.282 | −22.198 | 1.00 | 224.57 |
| 6763 | OE2 | GLU | E | 82 | −4.216 | 60.166 | −24.380 | 1.00 | 224.57 |
| 6764 | C | GLU | E | 82 | −1.623 | 64.749 | −23.596 | 1.00 | 99.21 |
| 6765 | O | GLU | E | 82 | −2.287 | 65.700 | −23.142 | 1.00 | 99.21 |
| 6766 | N | VAL | E | 83 | −1.231 | 64.670 | −24.876 | 1.00 | 128.82 |
| 6767 | CA | VAL | E | 83 | −1.599 | 65.690 | −25.871 | 1.00 | 128.82 |
| 6768 | CB | VAL | E | 83 | −0.388 | 66.250 | −26.586 | 1.00 | 97.06 |
| 6769 | CG1 | VAL | E | 83 | −0.835 | 67.323 | −27.572 | 1.00 | 97.06 |
| 6770 | CG2 | VAL | E | 83 | 0.585 | 66.824 | −25.562 | 1.00 | 97.06 |
| 6771 | C | VAL | E | 83 | −2.564 | 65.129 | −26.915 | 1.00 | 128.82 |
| 6772 | O | VAL | E | 83 | −2.424 | 63.998 | −27.378 | 1.00 | 128.82 |
| 6773 | N | PHE | E | 84 | −3.528 | 65.951 | −27.302 | 1.00 | 126.16 |
| 6774 | CA | PHE | E | 84 | −4.572 | 65.524 | −28.218 | 1.00 | 126.16 |
| 6775 | CB | PHE | E | 84 | −5.893 | 65.416 | −27.473 | 1.00 | 129.94 |
| 6776 | CG | PHE | E | 84 | −5.885 | 64.434 | −26.357 | 1.00 | 129.94 |
| 6777 | CD1 | PHE | E | 84 | −5.361 | 64.755 | −25.107 | 1.00 | 129.94 |
| 6778 | CD2 | PHE | E | 84 | −6.432 | 63.191 | −26.547 | 1.00 | 129.94 |
| 6779 | CE1 | PHE | E | 84 | −5.372 | 63.832 | −24.070 | 1.00 | 129.94 |
| 6780 | CE2 | PHE | E | 84 | −6.449 | 62.262 | −25.522 | 1.00 | 129.94 |
| 6781 | CZ | PHE | E | 84 | −5.924 | 62.585 | −24.278 | 1.00 | 129.94 |
| 6782 | C | PHE | E | 84 | −4.872 | 66.377 | −29.422 | 1.00 | 126.16 |
| 6783 | O | PHE | E | 84 | −4.588 | 67.571 | −29.466 | 1.00 | 126.16 |
| 6784 | N | SER | E | 85 | −5.510 | 65.732 | −30.385 | 1.00 | 167.68 |
| 6785 | CA | SER | E | 85 | −5.960 | 66.381 | −31.604 | 1.00 | 167.68 |
| 6786 | CB | SER | E | 85 | −5.136 | 65.957 | −32.815 | 1.00 | 221.67 |
| 6787 | OG | SER | E | 85 | −5.627 | 66.593 | −33.981 | 1.00 | 221.67 |
| 6788 | C | SER | E | 85 | −7.390 | 65.909 | −31.783 | 1.00 | 167.68 |
| 6789 | O | SER | E | 85 | −7.620 | 64.718 | −32.008 | 1.00 | 167.68 |
| 6790 | N | ASP | E | 86 | −8.342 | 66.840 | −31.661 | 1.00 | 124.68 |
| 6791 | CA | ASP | E | 86 | −9.766 | 66.530 | −31.793 | 1.00 | 124.68 |
| 6792 | CB | ASP | E | 86 | −10.199 | 65.550 | −30.697 | 1.00 | 146.90 |
| 6793 | CG | ASP | E | 86 | −11.214 | 64.542 | −31.189 | 1.00 | 146.90 |
| 6794 | OD1 | ASP | E | 86 | −12.235 | 64.955 | −31.792 | 1.00 | 146.90 |
| 6795 | OD2 | ASP | E | 86 | −10.979 | 63.329 | −30.978 | 1.00 | 146.90 |
| 6796 | C | ASP | E | 86 | −10.583 | 67.792 | −31.658 | 1.00 | 124.68 |
| 6797 | O | ASP | E | 86 | −10.064 | 68.815 | −31.244 | 1.00 | 124.68 |
| 6798 | N | TRP | E | 87 | −11.864 | 67.722 | −31.995 | 1.00 | 145.58 |
| 6799 | CA | TRP | E | 87 | −12.717 | 68.901 | −31.880 | 1.00 | 145.58 |
| 6800 | CB | TRP | E | 87 | −14.072 | 68.644 | −32.537 | 1.00 | 249.10 |
| 6801 | CG | TRP | E | 87 | −14.077 | 69.003 | −33.989 | 1.00 | 249.10 |
| 6802 | CD2 | TRP | E | 87 | −13.811 | 68.127 | −35.085 | 1.00 | 249.10 |
| 6803 | CE2 | TRP | E | 87 | −13.877 | 68.898 | −36.268 | 1.00 | 249.10 |
| 6804 | CE3 | TRP | E | 87 | −13.517 | 66.759 | −35.187 | 1.00 | 249.10 |
| 6805 | CD1 | TRP | E | 87 | −14.291 | 70.241 | −34.532 | 1.00 | 249.10 |
| 6806 | NE1 | TRP | E | 87 | −14.173 | 70.189 | −35.899 | 1.00 | 249.10 |
| 6807 | CZ2 | TRP | E | 87 | −13.663 | 68.354 | −37.538 | 1.00 | 249.10 |
| 6808 | CZ3 | TRP | E | 87 | −13.302 | 66.212 | −36.451 | 1.00 | 249.10 |
| 6809 | CH2 | TRP | E | 87 | −13.379 | 67.011 | −37.610 | 1.00 | 249.10 |
| 6810 | C | TRP | E | 87 | −12.890 | 69.314 | −30.433 | 1.00 | 145.58 |
| 6811 | O | TRP | E | 87 | −12.607 | 70.467 | −30.077 | 1.00 | 145.58 |
| 6812 | N | LEU | E | 88 | −13.345 | 68.377 | −29.605 | 1.00 | 109.18 |
| 6813 | CA | LEU | E | 88 | −13.521 | 68.658 | −28.190 | 1.00 | 109.18 |
| 6814 | CB | LEU | E | 88 | −15.001 | 68.601 | −27.819 | 1.00 | 123.07 |
| 6815 | CG | LEU | E | 88 | −15.885 | 69.659 | −28.476 | 1.00 | 123.07 |
| 6816 | CD1 | LEU | E | 83 | −17.294 | 69.581 | −27.913 | 1.00 | 123.07 |
| 6817 | CD2 | LEU | E | 88 | −15.326 | 71.049 | −28.223 | 1.00 | 123.07 |
| 6818 | C | LEU | E | 88 | −12.715 | 67.699 | −27.298 | 1.00 | 109.18 |
| 6819 | O | LEU | E | 88 | −12.590 | 66.501 | −27.598 | 1.00 | 109.18 |
| 6820 | N | LEU | E | 89 | −12.147 | 68.231 | −26.214 | 1.00 | 123.09 |
| 6821 | CA | LEU | E | 89 | −11.380 | 67.420 | −25.270 | 1.00 | 123.09 |
| 6822 | CB | LEU | E | 89 | −9.891 | 67.745 | −25.358 | 1.00 | 138.74 |
| 6823 | CG | LEU | E | 89 | −9.031 | 66.974 | −24.347 | 1.00 | 138.74 |
| 6824 | CD1 | LEU | E | 89 | −9.313 | 65.467 | −24.460 | 1.00 | 138.74 |
| 6825 | CD2 | LEU | E | 89 | −7.565 | 67.265 | −24.589 | 1.00 | 138.74 |
| 6826 | C | LEU | E | 89 | −11.865 | 67.724 | −23.864 | 1.00 | 123.09 |
| 6827 | O | LEU | E | 89 | −11.959 | 68.879 | −23.478 | 1.00 | 123.09 |
| 6828 | N | LEU | E | 90 | −12.184 | 66.695 | −23.097 | 1.00 | 99.50 |
| 6829 | CA | LEU | E | 90 | −12.659 | 66.912 | −21.737 | 1.00 | 99.50 |
| 6830 | CB | LEU | E | 90 | −13.556 | 65.759 | −21.305 | 1.00 | 113.63 |
| 6831 | CG | LEU | E | 90 | −13.932 | 65.784 | −19.817 | 1.00 | 113.63 |
| 6832 | CD1 | LEU | E | 90 | −14.685 | 67.054 | −19.527 | 1.00 | 113.63 |
| 6833 | CD2 | LEU | E | 90 | −14.770 | 64.565 | −19.457 | 1.00 | 113.63 |
| 6834 | C | LEU | E | 90 | −11.502 | 67.023 | −20.773 | 1.00 | 99.50 |
| 6835 | O | LEU | E | 90 | −10.779 | 66.046 | −20.570 | 1.00 | 99.50 |
| 6836 | N | GLN | E | 91 | −11.316 | 68.194 | −20.170 | 1.00 | 92.89 |
| 6837 | CA | GLN | E | 91 | −10.202 | 68.367 | −19.228 | 1.00 | 92.89 |
| 6838 | CB | GLN | E | 91 | −9.505 | 69.690 | −19.467 | 1.00 | 161.25 |
| 6839 | CG | GLN | E | 91 | −8.933 | 69.836 | −20.848 | 1.00 | 161.25 |
| 6840 | CD | GLN | E | 91 | −8.254 | 71.177 | −21.039 | 1.00 | 161.25 |
| 6841 | OE1 | GLN | E | 91 | −8.877 | 72.232 | −20.866 | 1.00 | 161.25 |
| 6842 | NE2 | GLN | E | 91 | −6.968 | 71.149 | −21.398 | 1.00 | 161.25 |
| 6843 | C | GLN | E | 91 | −10.632 | 68.289 | −17.770 | 1.00 | 92.89 |
| 6844 | O | GLN | E | 91 | −11.680 | 68.833 | −17.391 | 1.00 | 92.89 |
| 6845 | N | ALA | E | 92 | −9.814 | 67.612 | −16.961 | 1.00 | 113.22 |
| 6846 | CA | ALA | E | 92 | −10.114 | 67.458 | −15.544 | 1.00 | 113.22 |
| 6847 | CB | ALA | E | 92 | −10.393 | 65.997 | −15.212 | 1.00 | 187.04 |
| 6848 | C | ALA | E | 92 | −8.942 | 67.975 | −14.720 | 1.00 | 113.22 |
| 6849 | O | ALA | E | 92 | −7.777 | 67.802 | −15.105 | 1.00 | 113.22 |
| 6850 | N | SER | E | 93 | −9.271 | 68.623 | −13.596 | 1.00 | 114.34 |
| 6851 | CA | SER | E | 93 | −8.279 | 69.178 | −12.676 | 1.00 | 114.34 |
| 6852 | CB | SER | E | 93 | −8.973 | 69.762 | −11.425 | 1.00 | 137.73 |
| 6853 | OG | SER | E | 93 | −9.879 | 68.858 | −10.813 | 1.00 | 137.73 |
| 6854 | C | SER | E | 93 | −7.326 | 68.043 | −12.300 | 1.00 | 114.34 |
| 6855 | O | SER | E | 93 | −6.140 | 68.078 | −12.593 | 1.00 | 114.34 |
| 6856 | N | ALA | E | 94 | −7.869 | 67.028 | −11.653 | 1.00 | 121.13 |
| 6857 | CA | ALA | E | 94 | −7.113 | 65.858 | −11.255 | 1.00 | 121.13 |
| 6858 | CB | ALA | E | 94 | −6.898 | 65.855 | −9.748 | 1.00 | 206.55 |
| 6859 | C | ALA | E | 94 | −8.008 | 64.684 | −11.671 | 1.00 | 121.13 |
| 6860 | O | ALA | E | 94 | −9.235 | 64.832 | −11.768 | 1.00 | 121.13 |
| 6861 | N | GLU | E | 95 | −7.424 | 63.520 | −11.937 | 1.00 | 135.32 |
| 6862 | CA | GLU | E | 95 | −8.238 | 62.378 | −12.347 | 1.00 | 135.32 |
| 6863 | CB | GLU | E | 95 | −7.496 | 61.564 | −13.390 | 1.00 | 189.07 |
| 6864 | CG | GLU | E | 95 | −7.262 | 62.342 | −14.660 | 1.00 | 189.07 |
| 6865 | CD | GLU | E | 95 | −6.736 | 61.482 | −15.780 | 1.00 | 189.07 |
| 6866 | OE1 | GLU | E | 95 | −6.466 | 62.025 | −16.876 | 1.00 | 189.07 |
| 6867 | OE2 | GLU | E | 95 | −6.596 | 60.259 | −15.563 | 1.00 | 189.07 |
| 6868 | C | GLU | E | 95 | −8.632 | 61.500 | −11.171 | 1.00 | 135.32 |
| 6869 | O | GLU | E | 95 | −9.632 | 60.772 | −11.247 | 1.00 | 135.32 |
| 6870 | N | VAL | E | 96 | −7.854 | 61.581 | −10.085 | 1.00 | 121.52 |
| 6871 | CA | VAL | E | 96 | −8.118 | 60.807 | −8.865 | 1.00 | 121.52 |
| 6872 | CB | VAL | E | 96 | −6.994 | 59.808 | −8.591 | 1.00 | 138.26 |
| 6873 | CG1 | VAL | E | 96 | −7.480 | 58.754 | −7.615 | 1.00 | 138.26 |
| 6874 | CG2 | VAL | E | 96 | −6.534 | 59.178 | −9.895 | 1.00 | 138.26 |
| 6875 | C | VAL | E | 96 | −8.237 | 61.741 | −7.664 | 1.00 | 121.52 |
| 6876 | O | VAL | E | 96 | −7.376 | 62.598 | −7.462 | 1.00 | 121.52 |
| 6877 | N | VAL | E | 97 | −9.278 | 61.566 | −6.854 | 1.00 | 165.05 |
| 6878 | CA | VAL | E | 97 | −9.458 | 62.462 | −5.725 | 1.00 | 165.05 |
| 6879 | CB | VAL | E | 97 | −10.499 | 63.511 | −6.048 | 1.00 | 119.89 |
| 6880 | CG1 | VAL | E | 97 | −10.338 | 64.666 | −5.113 | 1.00 | 119.89 |
| 6881 | CG2 | VAL | E | 97 | −10.373 | 63.961 | −7.482 | 1.00 | 119.89 |
| 6882 | C | VAL | E | 97 | −9.856 | 61.866 | −4.380 | 1.00 | 165.05 |
| 6883 | O | VAL | E | 97 | −10.545 | 60.844 | −4.325 | 1.00 | 165.05 |
| 6884 | N | MET | E | 98 | −9.427 | 62.544 | −3.306 | 1.00 | 159.57 |
| 6885 | CA | MET | E | 98 | −9.713 | 62.161 | −1.914 | 1.00 | 159.57 |
| 6886 | CB | MET | E | 98 | −8.657 | 62.750 | −0.973 | 1.00 | 249.69 |
| 6887 | CG | MET | E | 98 | −7.247 | 62.228 | −1.152 | 1.00 | 249.69 |
| 6888 | SD | MET | E | 98 | −7.038 | 60.577 | −0.463 | 1.00 | 249.69 |
| 6889 | CE | MET | E | 98 | −6.946 | 60.948 | 1.288 | 1.00 | 249.69 |
| 6890 | C | MET | E | 98 | −11.071 | 62.740 | −1.522 | 1.00 | 159.57 |
| 6891 | O | MET | E | 98 | −11.268 | 63.954 | −1.606 | 1.00 | 159.57 |
| 6892 | N | GLU | E | 99 | −11.993 | 61.888 | −1.075 | 1.00 | 145.00 |
| 6893 | CA | GLU | E | 99 | −13.327 | 62.349 | −0.683 | 1.00 | 145.00 |
| 6894 | CB | GLU | E | 99 | −13.989 | 61.342 | 0.261 | 1.00 | 208.72 |
| 6895 | CG | GLU | E | 99 | −15.505 | 61.399 | 0.247 | 1.00 | 208.72 |
| 6896 | CD | GLU | E | 99 | −16.126 | 60.618 | 1.385 | 1.00 | 208.72 |
| 6897 | OE1 | GLU | E | 99 | −15.593 | 59.542 | 1.731 | 1.00 | 208.72 |
| 6898 | OE2 | GLU | E | 99 | −17.154 | 61.079 | 1.923 | 1.00 | 208.72 |
| 6899 | C | GLU | E | 99 | −13.241 | 63.699 | 0.022 | 1.00 | 145.00 |
| 6900 | O | GLU | E | 99 | −12.518 | 63.854 | 0.993 | 1.00 | 145.00 |
| 6901 | N | GLY | E | 100 | −13.970 | 64.686 | −0.473 | 1.00 | 140.46 |
| 6902 | CA | GLY | E | 100 | −13.941 | 65.989 | 0.164 | 1.00 | 140.46 |
| 6903 | C | GLY | E | 100 | −13.192 | 67.070 | −0.585 | 1.00 | 140.46 |
| 6904 | O | GLY | E | 100 | −13.449 | 68.256 | −0.353 | 1.00 | 140.46 |
| 6905 | N | GLN | E | 101 | −12.279 | 66.679 | −1.475 | 1.00 | 158.90 |
| 6906 | CA | GLN | E | 101 | −11.493 | 67.641 | −2.259 | 1.00 | 158.90 |
| 6907 | CB | GLN | E | 101 | −10.255 | 66.969 | −2.835 | 1.00 | 248.74 |
| 6908 | CG | GLN | E | 101 | −9.216 | 66.625 | −1.800 | 1.00 | 248.74 |
| 6909 | CD | GLN | E | 101 | −9.002 | 67.764 | −0.831 | 1.00 | 248.74 |
| 6910 | OE1 | GLN | E | 101 | −9.873 | 68.070 | −0.017 | 1.00 | 248.74 |
| 6911 | NE2 | GLN | E | 101 | −7.848 | 68.409 | −0.919 | 1.00 | 248.74 |
| 6912 | C | GLN | E | 101 | −12.290 | 68.324 | −3.371 | 1.00 | 158.90 |
| 6913 | O | GLN | E | 101 | −13.445 | 67.987 | −3.628 | 1.00 | 158.90 |
| 6914 | N | PRO | E | 102 | −11.657 | 69.302 | −4.050 | 1.00 | 164.28 |
| 6915 | CD | PRO | E | 102 | −10.406 | 69.971 | −3.720 | 1.00 | 154.28 |
| 6916 | CA | PRO | E | 102 | −12.358 | 69.987 | −5.140 | 1.00 | 164.28 |
| 6917 | CB | PRO | E | 102 | −11.739 | 71.379 | −5.074 | 1.00 | 154.28 |
| 6918 | CG | PRO | E | 102 | −10.312 | 71.040 | −4.796 | 1.00 | 154.28 |
| 6919 | C | PRO | E | 102 | −12.161 | 69.327 | −6.496 | 1.00 | 164.28 |
| 6920 | O | PRO | E | 102 | −11.119 | 68.720 | −6.771 | 1.00 | 164.28 |
| 6921 | N | LEU | E | 103 | −13.169 | 69.467 | −7.358 | 1.00 | 176.34 |
| 6922 | CA | LEU | E | 103 | −13.127 | 68.873 | −8.679 | 1.00 | 176.34 |
| 6923 | CB | LEU | E | 103 | −13.983 | 67.617 | −8.690 | 1.00 | 122.49 |
| 6924 | CG | LEU | E | 103 | −13.722 | 66.849 | −9.971 | 1.00 | 122.49 |
| 6925 | CD1 | LEU | E | 103 | −12.310 | 66.308 | −9.883 | 1.00 | 122.49 |
| 6926 | CD2 | LEU | E | 103 | −14.724 | 65.736 | −10.156 | 1.00 | 122.49 |
| 6927 | C | LEU | E | 103 | −13.618 | 69.805 | −9.776 | 1.00 | 176.34 |
| 6928 | O | LEU | E | 103 | −14.736 | 70.305 | −9.696 | 1.00 | 176.34 |
| 6929 | N | PHE | E | 104 | −12.805 | 70.027 | −10.806 | 1.00 | 126.68 |
| 6930 | CA | PHE | E | 104 | −13.233 | 70.890 | −11.903 | 1.00 | 126.68 |
| 6931 | CB | PHE | E | 104 | −12.412 | 72.174 | −11.954 | 1.00 | 239.90 |
| 6932 | CG | PHE | E | 104 | −12.405 | 72.934 | −10.681 | 1.00 | 239.90 |
| 6933 | CD1 | PHE | E | 104 | −11.612 | 72.520 | −9.623 | 1.00 | 239.90 |
| 6934 | CD2 | PHE | E | 104 | −13.201 | 74.060 | −10.528 | 1.00 | 239.90 |
| 6935 | CE1 | PHE | E | 104 | −11.608 | 73.221 | −8.416 | 1.00 | 239.90 |
| 6936 | CE2 | PHE | E | 104 | −13.211 | 74.770 | −9.331 | 1.00 | 239.90 |
| 6937 | CZ | PHE | E | 104 | −12.410 | 74.349 | −8.269 | 1.00 | 239.90 |
| 6938 | C | PHE | E | 104 | −13.110 | 70.197 | −13.250 | 1.00 | 126.68 |
| 6939 | O | PHE | E | 104 | −12.033 | 69.723 | −13.600 | 1.00 | 126.68 |
| 6940 | N | LEU | E | 105 | −14.208 | 70.136 | −14.003 | 1.00 | 132.08 |
| 6941 | CA | LEU | E | 105 | −14.176 | 69.524 | −15.327 | 1.00 | 132.08 |
| 6942 | CB | LEU | E | 105 | −15.249 | 68.452 | −15.456 | 1.00 | 106.14 |
| 6943 | CG | LEU | E | 105 | −15.131 | 67.347 | −14.414 | 1.00 | 106.14 |
| 6944 | CD1 | LEU | E | 105 | −16.174 | 66.283 | −14.719 | 1.00 | 106.14 |
| 6945 | CD2 | LEU | E | 105 | −13.704 | 66.777 | −14.429 | 1.00 | 106.14 |
| 6946 | C | LEU | E | 105 | −14.413 | 70.618 | −16.344 | 1.00 | 132.08 |
| 6947 | O | LEU | E | 105 | −15.119 | 71.592 | −16.064 | 1.00 | 132.08 |
| 6948 | N | ARG | E | 106 | −13.848 | 70.456 | −17.532 | 1.00 | 113.14 |
| 6949 | CA | ARG | E | 106 | −13.996 | 71.486 | −18.543 | 1.00 | 113.14 |
| 6950 | CB | ARG | E | 106 | −12.753 | 72.363 | −18.492 | 1.00 | 157.66 |
| 6951 | CG | ARG | E | 106 | −12.740 | 73.498 | −19.454 | 1.00 | 157.66 |
| 6952 | CD | ARG | E | 106 | −11.397 | 74.197 | −19.428 | 1.00 | 157.66 |
| 6953 | NE | ARG | E | 106 | −11.356 | 75.192 | −20.478 | 1.00 | 157.66 |
| 6954 | CZ | ARG | E | 106 | −10.256 | 75.566 | −21.103 | 1.00 | 157.66 |
| 6955 | NH1 | ARG | E | 106 | −9.094 | 75.020 | −20.774 | 1.00 | 157.66 |
| 6956 | NH2 | ARG | E | 106 | −10.331 | 76.473 | −22.071 | 1.00 | 157.66 |
| 6957 | C | ARG | E | 106 | −14.172 | 70.905 | −19.932 | 1.00 | 113.14 |
| 6958 | O | ARG | E | 106 | −13.363 | 70.068 | −20.365 | 1.00 | 113.14 |
| 6959 | N | CYS | E | 107 | −15.235 | 71.312 | −20.620 | 1.00 | 132.92 |
| 6960 | CA | CYS | E | 107 | −15.456 | 70.829 | −21.979 | 1.00 | 132.92 |
| 6961 | C | CYS | E | 107 | −14.646 | 71.786 | −22.808 | 1.00 | 132.92 |
| 6962 | O | CYS | E | 107 | −15.068 | 72.922 | −22.996 | 1.00 | 132.92 |
| 6963 | CB | CYS | E | 107 | −16.923 | 70.942 | −22.370 | 1.00 | 146.71 |
| 6964 | SG | CYS | E | 107 | −17.372 | 70.056 | −23.927 | 1.00 | 146.71 |
| 6965 | N | HIS | E | 108 | −13.483 | 71.339 | −23.282 | 1.00 | 154.76 |
| 6966 | CA | HIS | E | 108 | −12.576 | 72.195 | −24.052 | 1.00 | 154.76 |
| 6967 | CB | HIS | E | 108 | −11.130 | 71.911 | −23.639 | 1.00 | 172.76 |
| 6968 | CG | HIS | E | 108 | −10.136 | 72.910 | −24.161 | 1.00 | 172.76 |
| 6969 | CD2 | HIS | E | 108 | −8.957 | 72.738 | −24.810 | 1.00 | 172.76 |
| 6970 | ND1 | HIS | E | 108 | −10.277 | 74.262 | −23.967 | 1.00 | 172.76 |
| 6971 | CE1 | HIS | E | 108 | −9.221 | 74.888 | −24.473 | 1.00 | 172.76 |
| 6972 | NE2 | HIS | E | 108 | −8.409 | 73.987 | −24.986 | 1.00 | 172.76 |
| 6973 | C | HIS | E | 108 | −12.688 | 72.087 | −25.560 | 1.00 | 154.76 |
| 6974 | O | HIS | E | 108 | −12.576 | 70.997 | −26.129 | 1.00 | 154.76 |
| 6975 | N | GLY | E | 109 | −12.892 | 73.240 | −26.194 | 1.00 | 128.76 |
| 6976 | CA | GLY | E | 109 | −13.007 | 73.287 | −27.637 | 1.00 | 128.76 |
| 6977 | C | GLY | E | 109 | −11.632 | 73.334 | −28.260 | 1.00 | 128.76 |
| 6978 | O | GLY | E | 109 | −10.666 | 73.574 | −27.552 | 1.00 | 128.76 |
| 6979 | N | TRP | E | 110 | −11.539 | 73.092 | −29.566 | 1.00 | 154.27 |
| 6980 | CA | TRP | E | 110 | −10.260 | 73.136 | −30.251 | 1.00 | 154.27 |
| 6981 | CB | TRP | E | 110 | −10.312 | 72.268 | −31.503 | 1.00 | 170.55 |
| 6982 | CG | TRP | E | 110 | −9.107 | 72.411 | −32.397 | 1.00 | 170.55 |
| 6983 | CD2 | TRP | E | 110 | −7.963 | 71.539 | −32.468 | 1.00 | 170.55 |
| 6984 | CE2 | TRP | E | 110 | −7.073 | 72.098 | −33.406 | 1.00 | 170.55 |
| 6985 | CE3 | TRP | E | 110 | −7.606 | 70.345 | −31.825 | 1.00 | 170.55 |
| 6986 | CD1 | TRP | E | 110 | −8.863 | 73.422 | −33.270 | 1.00 | 170.55 |
| 6987 | NE1 | TRP | E | 110 | −7.645 | 73.242 | −33.877 | 1.00 | 170.55 |
| 6988 | CZ2 | TRP | E | 110 | −5.840 | 71.501 | −33.719 | 1.00 | 170.55 |
| 6989 | CZ3 | TRP | E | 110 | −6.375 | 69.755 | −32.137 | 1.00 | 170.55 |
| 6990 | CH2 | TRP | E | 110 | −5.509 | 70.339 | −33.077 | 1.00 | 170.55 |
| 6991 | C | TRP | E | 110 | −9.897 | 74.586 | −30.600 | 1.00 | 154.27 |
| 6992 | O | TRP | E | 110 | −10.786 | 75.431 | −30.767 | 1.00 | 154.27 |
| 6993 | N | ARG | E | 111 | −8.596 | 74.878 | −30.693 | 1.00 | 180.74 |
| 6994 | CA | ARG | E | 111 | −8.116 | 76.229 | −30.994 | 1.00 | 180.74 |
| 6995 | CB | ARG | E | 111 | −8.361 | 76.580 | −32.460 | 1.00 | 249.46 |
| 6996 | CG | ARG | E | 111 | −7.220 | 76.198 | −33.381 | 1.00 | 249.46 |
| 6997 | CD | ARG | E | 111 | −7.366 | 76.870 | −34.738 | 1.00 | 249.46 |
| 6998 | NE | ARG | E | 111 | −6.076 | 77.324 | −35.248 | 1.00 | 249.46 |
| 6999 | CZ | ARG | E | 111 | −5.295 | 78.200 | −34.623 | 1.00 | 249.46 |
| 7000 | NH1 | ARG | E | 111 | −5.672 | 78.720 | −33.463 | 1.00 | 249.46 |
| 7001 | NH2 | ARG | E | 111 | −4.136 | 78.557 | −35.155 | 1.00 | 249.46 |
| 7002 | C | ARG | E | 111 | −8.804 | 77.263 | −30.104 | 1.00 | 180.74 |
| 7003 | O | ARG | E | 111 | −9.013 | 78.408 | −30.490 | 1.00 | 180.74 |
| 7004 | N | ASN | E | 112 | −9.156 | 76.839 | −28.903 | 1.00 | 179.60 |
| 7005 | CA | ASN | E | 112 | −9.815 | 77.702 | −27.949 | 1.00 | 179.60 |
| 7006 | CB | ASN | E | 112 | −8.820 | 78.719 | −27.388 | 1.00 | 235.58 |
| 7007 | CG | ASN | E | 112 | −9.314 | 79.373 | −26.110 | 1.00 | 235.58 |
| 7008 | OD1 | ASN | E | 112 | −10.493 | 79.276 | −25.757 | 1.00 | 235.58 |
| 7009 | ND2 | ASN | E | 112 | −8.413 | 80.052 | −25.412 | 1.00 | 235.58 |
| 7010 | C | ASN | E | 112 | −11.002 | 78.426 | −28.579 | 1.00 | 179.60 |
| 7011 | O | ASN | E | 112 | −11.299 | 79.551 | −28.193 | 1.00 | 179.60 |
| 7012 | N | TRP | E | 113 | −11.671 | 77.798 | −29.550 | 1.00 | 181.35 |
| 7013 | CA | TRP | E | 113 | −12.841 | 78.420 | −30.174 | 1.00 | 181.35 |
| 7014 | CB | TRP | E | 113 | −13.343 | 77.633 | −31.368 | 1.00 | 198.74 |
| 7015 | CG | TRP | E | 113 | −12.618 | 77.902 | −32.604 | 1.00 | 198.74 |
| 7016 | CD2 | TRP | E | 113 | −12.345 | 76.965 | −33.651 | 1.00 | 198.74 |
| 7017 | CE2 | TRP | E | 113 | −11.666 | 77.674 | −34.676 | 1.00 | 198.74 |
| 7018 | CE3 | TRP | E | 113 | −12.603 | 75.599 | −33.823 | 1.00 | 198.74 |
| 7019 | CD1 | TRP | E | 113 | −12.114 | 79.099 | −33.018 | 1.00 | 198.74 |
| 7020 | NE1 | TRP | E | 113 | −11.537 | 78.973 | −34.267 | 1.00 | 198.74 |
| 7021 | CZ2 | TRP | E | 113 | −11.246 | 77.052 | −35.861 | 1.00 | 198.74 |
| 7022 | CZ3 | TRP | E | 113 | −12.185 | 74.983 | −34.994 | 1.00 | 198.74 |
| 7023 | CH2 | TRP | E | 113 | −11.515 | 75.711 | −36.001 | 1.00 | 198.74 |
| 7024 | C | TRP | E | 113 | −13.968 | 78.478 | −29.164 | 1.00 | 181.35 |
| 7025 | O | TRP | E | 113 | −13.763 | 78.229 | −27.974 | 1.00 | 181.35 |
| 7026 | N | ASP | E | 114 | −15.168 | 78.806 | −29.629 | 1.00 | 198.96 |
| 7027 | CA | ASP | E | 114 | −16.312 | 78.881 | −28.724 | 1.00 | 198.96 |
| 7028 | CB | ASP | E | 114 | −17.015 | 80.250 | −28.852 | 1.00 | 241.05 |
| 7029 | CG | ASP | E | 114 | −16.337 | 81.350 | −28.026 | 1.00 | 241.05 |
| 7030 | OD1 | ASP | E | 114 | −16.217 | 81.193 | −26.790 | 1.00 | 241.05 |
| 7031 | OD2 | ASP | E | 114 | −15.928 | 82.374 | −28.614 | 1.00 | 241.05 |
| 7032 | C | ASP | E | 114 | −17.299 | 77.746 | −28.994 | 1.00 | 198.96 |
| 7033 | O | ASP | E | 114 | −17.646 | 77.470 | −30.151 | 1.00 | 198.96 |
| 7034 | N | VAL | E | 115 | −17.732 | 77.083 | −27.922 | 1.00 | 162.46 |
| 7035 | CA | VAL | E | 115 | −18.686 | 75.985 | −28.039 | 1.00 | 162.46 |
| 7036 | CB | VAL | E | 115 | −18.191 | 74.726 | −27.316 | 1.00 | 122.72 |
| 7037 | CG1 | VAL | E | 115 | −19.018 | 73.528 | −27.751 | 1.00 | 122.72 |
| 7038 | CG2 | VAL | E | 115 | −16.727 | 74.493 | −27.603 | 1.00 | 122.72 |
| 7039 | C | VAL | E | 115 | −20.033 | 76.382 | −27.434 | 1.00 | 162.46 |
| 7040 | O | VAL | E | 115 | −20.084 | 77.042 | −26.385 | 1.00 | 162.46 |
| 7041 | N | TYR | E | 116 | −21.114 | 75.972 | −28.096 | 1.00 | 116.01 |
| 7042 | CA | TYR | E | 116 | −22.468 | 76.285 | −27.648 | 1.00 | 116.01 |
| 7043 | CB | TYR | E | 116 | −23.177 | 77.143 | −28.693 | 1.00 | 231.08 |
| 7044 | CG | TYR | E | 116 | −22.540 | 78.498 | −28.877 | 1.00 | 231.08 |
| 7045 | CD1 | TYR | E | 116 | −21.643 | 78.743 | −29.918 | 1.00 | 231.08 |
| 7046 | CE1 | TYR | E | 116 | −21.024 | 79.992 | −30.062 | 1.00 | 231.08 |
| 7047 | CD2 | TYR | E | 116 | −22.805 | 79.531 | −27.984 | 1.00 | 231.08 |
| 7048 | CE2 | TYR | E | 116 | −22.194 | 80.780 | −28.114 | 1.00 | 231.08 |
| 7049 | CZ | TYR | E | 116 | −21.306 | 81.006 | −29.154 | 1.00 | 231.08 |
| 7050 | OH | TYR | E | 116 | −20.705 | 82.241 | −29.278 | 1.00 | 231.08 |
| 7051 | C | TYR | E | 116 | −23.279 | 75.007 | −27.387 | 1.00 | 116.01 |
| 7052 | O | TYR | E | 116 | −22.829 | 73.909 | −27.722 | 1.00 | 116.01 |
| 7053 | N | LYS | E | 117 | −24.472 | 75.163 | −26.792 | 1.00 | 118.22 |
| 7054 | CA | LYS | E | 117 | −25.359 | 74.042 | −26.454 | 1.00 | 118.22 |
| 7055 | CB | LYS | E | 117 | −26.062 | 73.531 | −27.701 | 1.00 | 223.92 |
| 7056 | CG | LYS | E | 117 | −27.319 | 74.305 | −28.079 | 1.00 | 223.92 |
| 7057 | CD | LYS | E | 117 | −28.221 | 73.481 | −29.013 | 1.00 | 223.92 |
| 7058 | CE | LYS | E | 117 | −28.639 | 72.150 | −28.354 | 1.00 | 223.92 |
| 7059 | NZ | LYS | E | 117 | −29.508 | 71.267 | −29.203 | 1.00 | 223.92 |
| 7060 | C | LYS | E | 117 | −24.619 | 72.877 | −25.764 | 1.00 | 118.22 |
| 7061 | O | LYS | E | 117 | −24.736 | 71.712 | −26.150 | 1.00 | 118.22 |
| 7062 | N | VAL | E | 118 | −23.883 | 73.200 | −24.714 | 1.00 | 129.60 |
| 7063 | CA | VAL | E | 118 | −23.120 | 72.205 | −23.991 | 1.00 | 129.60 |
| 7064 | CB | VAL | E | 118 | −21.888 | 72.855 | −23.349 | 1.00 | 89.69 |
| 7065 | CG1 | VAL | E | 118 | −21.403 | 72.044 | −22.155 | 1.00 | 89.69 |
| 7066 | CG2 | VAL | E | 118 | −20.796 | 72.955 | −24.377 | 1.00 | 89.69 |
| 7067 | C | VAL | E | 118 | −23.869 | 71.403 | −22.939 | 1.00 | 129.60 |
| 7068 | O | VAL | E | 118 | −24.702 | 71.913 | −22.190 | 1.00 | 129.60 |
| 7069 | N | ILE | E | 119 | −23.522 | 70.124 | −22.896 | 1.00 | 95.90 |
| 7070 | CA | ILE | E | 119 | −24.087 | 69.164 | −21.965 | 1.00 | 95.90 |
| 7071 | CB | ILE | E | 119 | −25.146 | 68.311 | −22.666 | 1.00 | 119.63 |
| 7072 | CG2 | ILE | E | 119 | −25.826 | 67.375 | −21.651 | 1.00 | 119.63 |
| 7073 | CG1 | ILE | E | 119 | −26.147 | 69.225 | −23.373 | 1.00 | 119.63 |
| 7074 | CD1 | ILE | E | 119 | −26.848 | 68.560 | −24.500 | 1.00 | 119.63 |
| 7075 | C | ILE | E | 119 | −22.989 | 68.219 | −21.503 | 1.00 | 95.90 |
| 7076 | O | ILE | E | 119 | −22.248 | 67.666 | −22.322 | 1.00 | 95.90 |
| 7077 | N | TYR | E | 120 | −22.869 | 68.034 | −20.202 | 1.00 | 107.56 |
| 7078 | CA | TYR | E | 120 | −21.875 | 67.106 | −19.719 | 1.00 | 107.56 |
| 7079 | CB | TYR | E | 120 | −21.255 | 67.604 | −18.439 | 1.00 | 104.01 |
| 7080 | CG | TYR | E | 120 | −20.386 | 68.807 | −18.628 | 1.00 | 104.01 |
| 7081 | CD1 | TYR | E | 120 | −20.926 | 70.082 | −18.631 | 1.00 | 104.01 |
| 7082 | CE1 | TYR | E | 120 | −20.114 | 71.219 | −18.804 | 1.00 | 104.01 |
| 7083 | CD2 | TYR | E | 120 | −19.015 | 68.678 | −18.804 | 1.00 | 104.01 |
| 7084 | CE2 | TYR | E | 120 | −18.202 | 69.795 | −18.983 | 1.00 | 104.01 |
| 7085 | CZ | TYR | E | 120 | −18.752 | 71.061 | −18.983 | 1.00 | 104.01 |
| 7086 | OH | TYR | E | 120 | −17.933 | 72.151 | −19.184 | 1.00 | 104.01 |
| 7087 | C | TYR | E | 120 | −22.604 | 65.814 | −19.436 | 1.00 | 107.56 |
| 7088 | O | TYR | E | 120 | −23.806 | 65.843 | −19.141 | 1.00 | 107.56 |
| 7089 | N | TYR | E | 121 | −21.908 | 64.685 | −19.536 | 1.00 | 107.48 |
| 7090 | CA | TYR | E | 121 | −22.543 | 63.401 | −19.260 | 1.00 | 107.48 |
| 7091 | CB | TYR | E | 121 | −22.756 | 62.594 | −20.561 | 1.00 | 135.25 |
| 7092 | CG | TYR | E | 121 | −23.773 | 63.118 | −21.547 | 1.00 | 135.25 |
| 7093 | CD1 | TYR | E | 121 | −23.626 | 64.371 | −22.119 | 1.00 | 135.25 |
| 7094 | CE1 | TYR | E | 121 | −24.513 | 64.825 | −23.097 | 1.00 | 135.25 |
| 7095 | CD2 | TYR | E | 121 | −24.837 | 62.324 | −21.967 | 1.00 | 135.25 |
| 7096 | CE2 | TYR | E | 121 | −25.730 | 62.763 | −22.942 | 1.00 | 135.25 |
| 7097 | CZ | TYR | E | 121 | −25.567 | 64.011 | −23.507 | 1.00 | 135.25 |
| 7098 | OH | TYR | E | 121 | −26.438 | 64.440 | −24.498 | 1.00 | 135.25 |
| 7099 | C | TYR | E | 121 | −21.706 | 62.546 | −18.293 | 1.00 | 107.48 |
| 7100 | O | TYR | E | 121 | −20.476 | 62.430 | −18.440 | 1.00 | 107.48 |
| 7101 | N | LYS | E | 122 | −22.376 | 61.938 | −17.315 | 1.00 | 117.16 |
| 7102 | CA | LYS | E | 122 | −21.694 | 61.059 | −16.384 | 1.00 | 117.16 |
| 7103 | CB | LYS | E | 122 | −21.760 | 61.615 | −14.969 | 1.00 | 184.34 |
| 7104 | CG | LYS | E | 122 | −21.046 | 60.734 | −13.965 | 1.00 | 184.34 |
| 7105 | CD | LYS | E | 122 | −21.385 | 61.133 | −12.559 | 1.00 | 184.34 |
| 7106 | CE | LYS | E | 122 | −20.792 | 60.167 | −11.568 | 1.00 | 184.34 |
| 7107 | NZ | LYS | E | 122 | −21.266 | 60.513 | −10.209 | 1.00 | 184.34 |
| 7108 | C | LYS | E | 122 | −22.391 | 59.709 | −16.426 | 1.00 | 117.16 |
| 7109 | O | LYS | E | 122 | −23.564 | 59.597 | −16.064 | 1.00 | 117.16 |
| 7110 | N | ASP | E | 123 | −21.669 | 58.684 | −16.864 | 1.00 | 145.37 |
| 7111 | CA | ASP | E | 123 | −22.228 | 57.333 | −16.960 | 1.00 | 145.37 |
| 7112 | CB | ASP | E | 123 | −22.532 | 56.766 | −15.574 | 1.00 | 150.82 |
| 7113 | CG | ASP | E | 123 | −21.271 | 56.378 | −14.821 | 1.00 | 150.82 |
| 7114 | OD1 | ASP | E | 123 | −20.424 | 55.649 | −15.391 | 1.00 | 150.82 |
| 7115 | OD2 | ASP | E | 123 | −21.128 | 56.795 | −13.657 | 1.00 | 150.82 |
| 7116 | C | ASP | E | 123 | −23.483 | 57.247 | −17.825 | 1.00 | 145.37 |
| 7117 | O | ASP | E | 123 | −24.508 | 56.696 | −17.395 | 1.00 | 145.37 |
| 7118 | N | GLY | E | 124 | −23.389 | 57.796 | −19.040 | 1.00 | 162.19 |
| 7119 | CA | GLY | E | 124 | −24.497 | 57.764 | −19.983 | 1.00 | 162.19 |
| 7120 | C | GLY | E | 124 | −25.683 | 58.666 | −19.698 | 1.00 | 162.19 |
| 7121 | O | GLY | E | 124 | −26.586 | 58.768 | −20.526 | 1.00 | 162.19 |
| 7122 | N | GLU | E | 125 | −25.683 | 59.324 | −18.541 | 1.00 | 143.04 |
| 7123 | CA | GLU | E | 125 | −26.776 | 60.216 | −18.134 | 1.00 | 143.04 |
| 7124 | CB | GLU | E | 125 | −27.041 | 60.063 | −16.627 | 1.00 | 249.69 |
| 7125 | CG | GLU | E | 125 | −27.627 | 58.724 | −16.208 | 1.00 | 249.69 |
| 7126 | CD | GLU | E | 125 | −29.094 | 58.592 | −16.573 | 1.00 | 249.69 |
| 7127 | OE1 | GLU | E | 125 | −29.901 | 59.390 | −16.051 | 1.00 | 249.69 |
| 7128 | OE2 | GLU | E | 125 | −29.440 | 57.696 | −17.379 | 1.00 | 249.69 |
| 7129 | C | GLU | E | 125 | −26.510 | 61.692 | −18.437 | 1.00 | 143.04 |
| 7130 | O | GLU | E | 125 | −25.384 | 62.166 | −18.309 | 1.00 | 143.04 |
| 7131 | N | ALA | E | 126 | −27.550 | 62.414 | −18.843 | 1.00 | 144.33 |
| 7132 | CA | ALA | E | 126 | −27.416 | 63.838 | −19.120 | 1.00 | 144.33 |
| 7133 | CB | ALA | E | 126 | −28.693 | 64.365 | −19.726 | 1.00 | 160.82 |
| 7134 | C | ALA | E | 126 | −27.187 | 64.451 | −17.754 | 1.00 | 144.33 |
| 7135 | O | ALA | E | 126 | −27.835 | 64.054 | −16.791 | 1.00 | 144.33 |
| 7136 | N | LEU | E | 127 | −26.285 | 65.419 | −17.645 | 1.00 | 143.63 |
| 7137 | CA | LEU | E | 127 | −26.002 | 65.998 | −16.319 | 1.00 | 143.63 |
| 7138 | CB | LEU | E | 127 | −24.565 | 65.687 | −15.904 | 1.00 | 101.20 |
| 7139 | CG | LEU | E | 127 | −24.442 | 65.621 | −14.395 | 1.00 | 101.20 |
| 7140 | CD1 | LEU | E | 127 | −25.446 | 64.623 | −13.859 | 1.00 | 101.20 |
| 7141 | CD2 | LEU | E | 127 | −23.029 | 65.214 | −14.036 | 1.00 | 101.20 |
| 7142 | C | LEU | E | 127 | −26.247 | 67.484 | −16.121 | 1.00 | 143.63 |
| 7143 | O | LEU | E | 127 | −27.036 | 67.875 | −15.264 | 1.00 | 143.63 |
| 7144 | N | LYS | E | 128 | −25.532 | 68.305 | −16.880 | 1.00 | 117.01 |
| 7145 | CA | LYS | E | 128 | −25.707 | 69.747 | −16.812 | 1.00 | 117.01 |
| 7146 | CB | LYS | E | 128 | −24.508 | 70.394 | −16.141 | 1.00 | 217.61 |
| 7147 | CG | LYS | E | 128 | −24.263 | 69.930 | −14.718 | 1.00 | 217.61 |
| 7148 | CD | LYS | E | 128 | −25.300 | 70.474 | −13.748 | 1.00 | 217.61 |
| 7149 | CE | LYS | E | 128 | −24.958 | 70.073 | −12.316 | 1.00 | 217.61 |
| 7150 | NZ | LYS | E | 128 | −25.780 | 70.793 | −11.306 | 1.00 | 217.61 |
| 7151 | C | LYS | E | 128 | −25.842 | 70.272 | −18.245 | 1.00 | 117.01 |
| 7152 | O | LYS | E | 128 | −25.417 | 69.599 | −19.194 | 1.00 | 117.01 |
| 7153 | N | TYR | E | 129 | −26.424 | 71.461 | −18.406 | 1.00 | 145.51 |
| 7154 | CA | TYR | E | 129 | −26.601 | 72.029 | −19.736 | 1.00 | 145.51 |
| 7155 | CB | TYR | E | 129 | −27.928 | 71.565 | −20.322 | 1.00 | 135.80 |
| 7156 | CG | TYR | E | 129 | −28.368 | 72.425 | −21.479 | 1.00 | 135.80 |
| 7157 | CD1 | TYR | E | 129 | −27.913 | 72.180 | −22.768 | 1.00 | 135.80 |
| 7158 | CE1 | TYR | E | 129 | −28.292 | 73.003 | −23.829 | 1.00 | 135.80 |
| 7159 | CD2 | TYR | E | 129 | −29.214 | 73.521 | −21.273 | 1.00 | 135.80 |
| 7160 | CE2 | TYR | E | 129 | −29.599 | 74.344 | −22.318 | 1.00 | 135.80 |
| 7161 | CZ | TYR | E | 129 | −29.135 | 74.078 | −23.595 | 1.00 | 135.80 |
| 7162 | OH | TYR | E | 129 | −29.523 | 74.878 | −24.643 | 1.00 | 135.80 |
| 7163 | C | TYR | E | 129 | −26.557 | 73.557 | −19.774 | 1.00 | 145.51 |
| 7164 | O | TYR | E | 129 | −27.124 | 74.221 | −18.907 | 1.00 | 145.51 |
| 7165 | N | TRP | E | 130 | −25.900 | 74.105 | −20.800 | 1.00 | 157.70 |
| 7166 | CA | TRP | E | 130 | −25.786 | 75.554 | −20.976 | 1.00 | 157.70 |
| 7167 | CB | TRP | E | 130 | −24.539 | 76.108 | −20.279 | 1.00 | 223.39 |
| 7168 | CG | TRP | E | 130 | −24.287 | 75.631 | −18.878 | 1.00 | 223.39 |
| 7169 | CD2 | TRP | E | 130 | −24.485 | 76.370 | −17.677 | 1.00 | 223.39 |
| 7170 | CE2 | TRP | E | 130 | −24.066 | 75.557 | −16.598 | 1.00 | 223.39 |
| 7171 | CE3 | TRP | E | 130 | −24.971 | 77.660 | −17.393 | 1.00 | 223.39 |
| 7172 | CD1 | TRP | E | 130 | −23.782 | 74.419 | −18.498 | 1.00 | 223.39 |
| 7173 | NE1 | TRP | E | 130 | −23.638 | 74.364 | −17.134 | 1.00 | 223.39 |
| 7174 | CZ2 | TRP | E | 130 | −24.117 | 75.974 | −15.268 | 1.00 | 223.39 |
| 7175 | CZ3 | TRP | E | 130 | −25.023 | 78.083 | −16.063 | 1.00 | 223.39 |
| 7176 | CH2 | TRP | E | 130 | −24.605 | 77.238 | −15.018 | 1.00 | 223.39 |
| 7177 | C | TRP | E | 130 | −25.699 | 75.944 | −22.451 | 1.00 | 157.70 |
| 7178 | O | TRP | E | 130 | −25.526 | 75.103 | −23.321 | 1.00 | 157.70 |
| 7179 | N | TYR | E | 131 | −25.812 | 77.239 | −22.718 | 1.00 | 154.17 |
| 7180 | CA | TYR | E | 131 | −25.721 | 77.775 | −24.074 | 1.00 | 154.17 |
| 7181 | CB | TYR | E | 131 | −26.551 | 79.040 | −24.193 | 1.00 | 200.28 |
| 7182 | CG | TYR | E | 131 | −26.730 | 79.484 | −25.605 | 1.00 | 200.28 |
| 7183 | CD1 | TYR | E | 131 | −27.576 | 78.794 | −26.465 | 1.00 | 200.28 |
| 7184 | CE1 | TYR | E | 131 | −27.722 | 79.186 | −27.789 | 1.00 | 200.28 |
| 7185 | CD2 | TYR | E | 131 | −26.028 | 80.579 | −26.100 | 1.00 | 200.28 |
| 7186 | CE2 | TYR | E | 131 | −26.166 | 80.979 | −27.426 | 1.00 | 200.28 |
| 7187 | CZ | TYR | E | 131 | −27.012 | 80.277 | −28.267 | 1.00 | 200.28 |
| 7188 | OH | TYR | E | 131 | −27.129 | 80.671 | −29.584 | 1.00 | 200.28 |
| 7189 | C | TYR | E | 131 | −24.238 | 78.105 | −24.250 | 1.00 | 154.17 |
| 7190 | O | TYR | E | 131 | −23.462 | 77.259 | −24.690 | 1.00 | 154.17 |
| 7191 | N | GLU | E | 132 | −23.848 | 79.340 | −23.930 | 1.00 | 210.53 |
| 7192 | CA | GLU | E | 132 | −22.436 | 79.713 | −23.979 | 1.00 | 210.53 |
| 7193 | CB | GLU | E | 132 | −22.234 | 81.158 | −23.507 | 1.00 | 249.69 |
| 7194 | CG | GLU | E | 132 | −22.565 | 82.244 | −24.531 | 1.00 | 249.69 |
| 7195 | CD | GLU | E | 132 | −21.342 | 83.062 | −24.912 | 1.00 | 249.69 |
| 7196 | OE1 | GLU | E | 132 | −20.307 | 82.939 | −24.219 | 1.00 | 249.69 |
| 7197 | OE2 | GLU | E | 132 | −21.411 | 83.832 | −25.895 | 1.00 | 249.69 |
| 7198 | C | GLU | E | 132 | −21.980 | 78.737 | −22.905 | 1.00 | 210.53 |
| 7199 | O | GLU | E | 132 | −22.554 | 78.723 | −21.809 | 1.00 | 210.53 |
| 7200 | N | ASN | E | 133 | −20.962 | 77.929 | −23.182 | 1.00 | 143.28 |
| 7201 | CA | ASN | E | 133 | −20.610 | 76.931 | −22.193 | 1.00 | 143.28 |
| 7202 | CB | ASN | E | 133 | −19.691 | 75.833 | −22.820 | 1.00 | 158.57 |
| 7203 | CG | ASN | E | 133 | −18.215 | 76.179 | −22.850 | 1.00 | 158.57 |
| 7204 | OD1 | ASN | E | 133 | −17.820 | 77.296 | −23.201 | 1.00 | 158.57 |
| 7205 | ND2 | ASN | E | 133 | −17.378 | 75.183 | −22.520 | 1.00 | 158.57 |
| 7206 | C | ASN | E | 133 | −20.152 | 77.365 | −20.796 | 1.00 | 143.28 |
| 7207 | O | ASN | E | 133 | −20.202 | 78.541 | −20.431 | 1.00 | 143.28 |
| 7208 | N | HIS | E | 134 | −19.772 | 76.381 | −19.995 | 1.00 | 154.96 |
| 7209 | CA | HIS | E | 134 | −19.363 | 76.622 | −18.635 | 1.00 | 154.96 |
| 7210 | CB | HIS | E | 134 | −20.574 | 76.432 | −17.724 | 1.00 | 249.69 |
| 7211 | CG | HIS | E | 134 | −20.319 | 76.834 | −16.296 | 1.00 | 249.69 |
| 7212 | CD2 | HIS | E | 134 | −20.339 | 76.096 | −15.160 | 1.00 | 249.69 |
| 7213 | ND1 | HIS | E | 134 | −19.981 | 78.107 | −15.945 | 1.00 | 249.69 |
| 7214 | CE1 | HIS | E | 134 | −19.792 | 78.160 | −14.625 | 1.00 | 249.69 |
| 7215 | NE2 | HIS | E | 134 | −20.003 | 76.958 | −14.134 | 1.00 | 249.69 |
| 7216 | C | HIS | E | 134 | −18.282 | 75.617 | −18.297 | 1.00 | 154.96 |
| 7217 | O | HIS | E | 134 | −17.703 | 74.988 | −19.184 | 1.00 | 154.96 |
| 7218 | N | ASN | E | 135 | −18.018 | 75.457 | −17.009 | 1.00 | 128.43 |
| 7219 | CA | ASN | E | 135 | −17.003 | 74.531 | −16.537 | 1.00 | 128.43 |
| 7220 | CB | ASN | E | 135 | −15.677 | 75.279 | −16.366 | 1.00 | 226.02 |
| 7221 | CG | ASN | E | 135 | −15.086 | 75.721 | −17.696 | 1.00 | 226.02 |
| 7222 | OD1 | ASN | E | 135 | −15.047 | 74.928 | −18.643 | 1.00 | 226.02 |
| 7223 | ND2 | ASN | E | 135 | −14.602 | 76.963 | −17.776 | 1.00 | 226.02 |
| 7224 | C | ASN | E | 135 | −17.436 | 73.896 | −15.226 | 1.00 | 128.43 |
| 7225 | O | ASN | E | 135 | −17.046 | 74.363 | −14.166 | 1.00 | 128.43 |
| 7226 | N | ILE | E | 136 | −18.253 | 72.842 | −15.318 | 1.00 | 149.25 |
| 7227 | CA | ILE | E | 136 | −18.788 | 72.102 | −14.159 | 1.00 | 149.25 |
| 7228 | CB | ILE | E | 136 | −19.268 | 70.698 | −14.588 | 1.00 | 170.48 |
| 7229 | CG2 | ILE | E | 136 | −18.140 | 69.955 | −15.266 | 1.00 | 170.48 |
| 7230 | CG1 | ILE | E | 136 | −19.748 | 69.897 | −13.378 | 1.00 | 170.48 |
| 7231 | CD1 | ILE | E | 136 | −20.169 | 68.485 | −13.726 | 1.00 | 170.48 |
| 7232 | C | ILE | E | 136 | −17.824 | 71.949 | −12.975 | 1.00 | 149.25 |
| 7233 | O | ILE | E | 136 | −16.894 | 71.133 | −13.008 | 1.00 | 149.25 |
| 7234 | N | SER | E | 137 | −18.096 | 72.718 | −11.918 | 1.00 | 150.66 |
| 7235 | CA | SER | E | 137 | −17.258 | 72.738 | −10.724 | 1.00 | 150.66 |
| 7236 | CB | SER | E | 137 | −16.914 | 74.185 | −10.367 | 1.00 | 213.03 |
| 7237 | OG | SER | E | 137 | −16.282 | 74.247 | −9.104 | 1.00 | 213.03 |
| 7238 | C | SER | E | 137 | −17.783 | 72.047 | −9.478 | 1.00 | 150.66 |
| 7239 | O | SER | E | 137 | −18.969 | 72.041 | −9.203 | 1.00 | 150.66 |
| 7240 | N | ILE | E | 138 | −16.853 | 71.504 | −8.709 | 1.00 | 157.82 |
| 7241 | CA | ILE | E | 138 | −17.154 | 70.795 | −7.483 | 1.00 | 157.82 |
| 7242 | CB | ILE | E | 138 | −17.060 | 69.286 | −7.712 | 1.00 | 122.12 |
| 7243 | CG2 | ILE | E | 138 | −17.033 | 68.548 | −6.388 | 1.00 | 122.12 |
| 7244 | CG1 | ILE | E | 138 | −18.240 | 68.834 | −8.550 | 1.00 | 122.12 |
| 7245 | CD1 | ILE | E | 138 | −18.110 | 67.429 | −9.027 | 1.00 | 122.12 |
| 7246 | C | ILE | E | 138 | −16.219 | 71.180 | −6.339 | 1.00 | 157.82 |
| 7247 | O | ILE | E | 138 | −15.000 | 71.039 | −6.435 | 1.00 | 157.82 |
| 7248 | N | THR | E | 139 | −16.813 | 71.655 | −5.251 | 1.00 | 216.52 |
| 7249 | CA | THR | E | 139 | −16.073 | 72.066 | −4.067 | 1.00 | 216.52 |
| 7250 | CB | THR | E | 139 | −16.922 | 73.033 | −3.250 | 1.00 | 203.55 |
| 7251 | OG1 | THR | E | 139 | −18.202 | 72.437 | −2.992 | 1.00 | 203.55 |
| 7252 | CG2 | THR | E | 139 | −17.135 | 74.324 | −4.026 | 1.00 | 203.55 |
| 7253 | C | THR | E | 139 | −15.745 | 70.839 | −3.224 | 1.00 | 216.52 |
| 7254 | O | THR | E | 139 | −14.637 | 70.307 | −3.273 | 1.00 | 216.52 |
| 7255 | N | ASN | E | 140 | −16.726 | 70.402 | −2.446 | 1.00 | 176.56 |
| 7256 | CA | ASN | E | 140 | −16.589 | 69.224 | −1.603 | 1.00 | 176.56 |
| 7257 | CB | ASN | E | 140 | −17.543 | 69.336 | −0.405 | 1.00 | 249.69 |
| 7258 | CG | ASN | E | 140 | −17.486 | 68.128 | 0.504 | 1.00 | 249.69 |
| 7259 | OD1 | ASN | E | 140 | −17.585 | 66.992 | 0.040 | 1.00 | 249.69 |
| 7260 | ND2 | ASN | E | 140 | −17.347 | 68.365 | 1.805 | 1.00 | 249.69 |
| 7261 | C | ASN | E | 140 | −16.974 | 68.036 | −2.490 | 1.00 | 176.56 |
| 7262 | O | ASN | E | 140 | −18.084 | 67.989 | −3.026 | 1.00 | 176.56 |
| 7263 | N | ALA | E | 141 | −16.060 | 67.084 | −2.648 | 1.00 | 151.69 |
| 7264 | CA | ALA | E | 141 | −16.311 | 65.924 | −3.498 | 1.00 | 151.69 |
| 7265 | CB | ALA | E | 141 | −15.045 | 65.594 | −4.302 | 1.00 | 113.23 |
| 7266 | C | ALA | E | 141 | −16.816 | 64.665 | −2.788 | 1.00 | 151.69 |
| 7267 | O | ALA | E | 141 | −16.218 | 64.183 | −1.826 | 1.00 | 151.69 |
| 7268 | N | THR | E | 142 | −17.925 | 64.133 | −3.292 | 1.00 | 151.96 |
| 7269 | CA | THR | E | 142 | −18.534 | 62.921 | −2.754 | 1.00 | 151.96 |
| 7270 | CB | THR | E | 142 | −20.050 | 62.908 | −2.974 | 1.00 | 230.06 |
| 7271 | OG1 | THR | E | 142 | −20.612 | 64.142 | −2.510 | 1.00 | 230.06 |
| 7272 | CG2 | THR | E | 142 | −20.678 | 61.760 | −2.218 | 1.00 | 230.06 |
| 7273 | C | THR | E | 142 | −17.947 | 61.753 | −3.520 | 1.00 | 151.96 |
| 7274 | O | THR | E | 142 | −17.415 | 61.923 | −4.625 | 1.00 | 151.96 |
| 7275 | N | VAL | E | 143 | −18.040 | 60.560 | −2.949 | 1.00 | 131.28 |
| 7276 | CA | VAL | E | 143 | −17.493 | 59.381 | −3.631 | 1.00 | 131.28 |
| 7277 | CB | VAL | E | 143 | −17.368 | 58.167 | −2.692 | 1.00 | 141.04 |
| 7278 | CG1 | VAL | E | 143 | −18.738 | 57.589 | −2.393 | 1.00 | 141.04 |
| 7279 | CG2 | VAL | E | 143 | −16.485 | 57.127 | −3.322 | 1.00 | 141.04 |
| 7280 | C | VAL | E | 143 | −18.402 | 58.987 | −4.781 | 1.00 | 131.28 |
| 7281 | O | VAL | E | 143 | −17.971 | 58.336 | −5.719 | 1.00 | 131.28 |
| 7282 | N | GLU | E | 144 | −19.667 | 59.388 | −4.697 | 1.00 | 177.47 |
| 7283 | CA | GLU | E | 144 | −20.614 | 59.063 | −5.746 | 1.00 | 177.47 |
| 7284 | CB | GLU | E | 144 | −22.048 | 59.323 | −5.298 | 1.00 | 249.69 |
| 7285 | CG | GLU | E | 144 | −22.470 | 58.500 | −4.105 | 1.00 | 249.69 |
| 7286 | CD | GLU | E | 144 | −22.694 | 59.353 | −2.886 | 1.00 | 249.69 |
| 7287 | OE1 | GLU | E | 144 | −23.589 | 60.221 | −2.939 | 1.00 | 249.69 |
| 7288 | OE2 | GLU | E | 144 | −21.977 | 59.167 | −1.880 | 1.00 | 249.69 |
| 7289 | C | GLU | E | 144 | −20.316 | 59.875 | −6.986 | 1.00 | 177.47 |
| 7290 | O | GLU | E | 144 | −20.847 | 59.583 | −8.050 | 1.00 | 177.47 |
| 7291 | N | ASP | E | 145 | −19.467 | 60.895 | −6.851 | 1.00 | 122.39 |
| 7292 | CA | ASP | E | 145 | −19.091 | 61.723 | −7.994 | 1.00 | 122.39 |
| 7293 | CB | ASP | E | 145 | −18.410 | 62.997 | −7.532 | 1.00 | 174.03 |
| 7294 | CG | ASP | E | 145 | −19.396 | 64.013 | −7.012 | 1.00 | 174.03 |
| 7295 | OD1 | ASP | E | 145 | −20.326 | 64.372 | −7.768 | 1.00 | 174.03 |
| 7296 | OD2 | ASP | E | 145 | −19.244 | 64.459 | −5.854 | 1.00 | 174.03 |
| 7297 | C | ASP | E | 145 | −18.165 | 60.969 | −8.943 | 1.00 | 122.39 |
| 7298 | O | ASP | E | 145 | −17.996 | 61.363 | −10.098 | 1.00 | 122.39 |
| 7299 | N | SER | E | 146 | −17.580 | 59.870 | −8.458 | 1.00 | 134.53 |
| 7300 | CA | SER | E | 146 | −16.672 | 59.031 | −9.263 | 1.00 | 134.53 |
| 7301 | CB | SER | E | 146 | −16.037 | 57.940 | −8.393 | 1.00 | 131.44 |
| 7302 | OG | SER | E | 146 | −15.340 | 58.481 | −7.281 | 1.00 | 131.44 |
| 7303 | C | SER | E | 146 | −17.412 | 58.362 | −10.418 | 1.00 | 134.53 |
| 7304 | O | SER | E | 146 | −18.431 | 57.729 | −10.211 | 1.00 | 134.53 |
| 7305 | N | GLY | E | 147 | −16.892 | 58.500 | −11.628 | 1.00 | 156.93 |
| 7306 | CA | GLY | E | 147 | −17.542 | 57.888 | −12.769 | 1.00 | 156.93 |
| 7307 | C | GLY | E | 147 | −16.839 | 58.176 | −14.083 | 1.00 | 156.93 |
| 7308 | O | GLY | E | 147 | −15.656 | 58.545 | −14.095 | 1.00 | 156.93 |
| 7309 | N | THR | E | 148 | −17.559 | 58.006 | −15.194 | 1.00 | 115.73 |
| 7310 | CA | THR | E | 148 | −16.993 | 58.263 | −16.530 | 1.00 | 115.73 |
| 7311 | CB | THR | E | 148 | −16.985 | 56.964 | −17.380 | 1.00 | 136.83 |
| 7312 | OG1 | THR | E | 148 | −18.127 | 56.934 | −18.238 | 1.00 | 136.83 |
| 7313 | CG2 | THR | E | 148 | −17.031 | 55.746 | −16.476 | 1.00 | 136.83 |
| 7314 | C | THR | E | 148 | −17.755 | 59.409 | −17.266 | 1.00 | 115.73 |
| 7315 | O | THR | E | 148 | −18.927 | 59.283 | −17.642 | 1.00 | 115.73 |
| 7316 | N | TYR | E | 149 | −17.068 | 60.533 | −17.457 | 1.00 | 98.03 |
| 7317 | CA | TYR | E | 149 | −17.660 | 61.699 | −18.084 | 1.00 | 98.03 |
| 7318 | CB | TYR | E | 149 | −17.292 | 62.968 | −17.301 | 1.00 | 106.49 |
| 7319 | CG | TYR | E | 149 | −17.670 | 62.986 | −15.828 | 1.00 | 106.49 |
| 7320 | CD1 | TYR | E | 149 | −16.951 | 62.244 | −14.885 | 1.00 | 106.49 |
| 7321 | CE1 | TYR | E | 149 | −17.293 | 62.279 | −13.545 | 1.00 | 106.49 |
| 7322 | CD2 | TYR | E | 149 | −18.740 | 63.766 | −15.378 | 1.00 | 106.49 |
| 7323 | CE2 | TYR | E | 149 | −19.081 | 63.812 | −14.046 | 1.00 | 106.49 |
| 7324 | CZ | TYR | E | 149 | −18.358 | 63.065 | −13.137 | 1.00 | 106.49 |
| 7325 | OH | TYR | E | 149 | −18.715 | 63.098 | −11.815 | 1.00 | 106.49 |
| 7326 | C | TYR | E | 149 | −17.229 | 61.914 | −19.518 | 1.00 | 98.03 |
| 7327 | O | TYR | E | 149 | −16.224 | 61.346 | −19.972 | 1.00 | 98.03 |
| 7328 | N | TYR | E | 150 | −18.002 | 62.767 | −20.200 | 1.00 | 87.55 |
| 7329 | CA | TYR | E | 150 | −17.780 | 63.194 | −21.595 | 1.00 | 87.55 |
| 7330 | CB | TYR | E | 150 | −18.019 | 62.028 | −22.591 | 1.00 | 125.81 |
| 7331 | CG | TYR | E | 150 | −19.456 | 61.684 | −22.936 | 1.00 | 125.81 |
| 7332 | CD1 | TYR | E | 150 | −20.224 | 62.527 | −23.739 | 1.00 | 125.81 |
| 7333 | CE1 | TYR | E | 150 | −21.551 | 62.200 | −24.087 | 1.00 | 125.81 |
| 7334 | CD2 | TYR | E | 150 | −20.041 | 60.496 | −22.482 | 1.00 | 125.81 |
| 7335 | CE2 | TYR | E | 150 | −21.371 | 60.157 | −22.828 | 1.00 | 125.81 |
| 7336 | CZ | TYR | E | 150 | −22.116 | 61.018 | −23.631 | 1.00 | 125.81 |
| 7337 | OH | TYR | E | 150 | −23.405 | 60.699 | −23.991 | 1.00 | 125.81 |
| 7338 | C | TYR | E | 150 | −18.765 | 64.338 | −21.835 | 1.00 | 87.55 |
| 7339 | O | TYR | E | 150 | −19.801 | 64.418 | −21.160 | 1.00 | 87.55 |
| 7340 | N | CYS | E | 151 | −18.456 | 65.235 | −22.763 | 1.00 | 108.53 |
| 7341 | CA | CYS | E | 151 | −19.370 | 66.343 | −23.043 | 1.00 | 108.53 |
| 7342 | C | CYS | E | 151 | −19.724 | 66.457 | −24.522 | 1.00 | 108.53 |
| 7343 | O | CYS | E | 151 | −19.030 | 65.919 | −25.385 | 1.00 | 108.53 |
| 7344 | CB | CYS | E | 151 | −18.749 | 67.647 | −22.588 | 1.00 | 127.42 |
| 7345 | SG | CYS | E | 151 | −17.166 | 68.090 | −23.414 | 1.00 | 127.42 |
| 7346 | N | THR | E | 152 | −20.816 | 67.151 | −24.810 | 1.00 | 109.73 |
| 7347 | CA | THR | E | 152 | −21.249 | 67.343 | −26.184 | 1.00 | 109.73 |
| 7348 | CB | THR | E | 152 | −22.546 | 66.577 | −26.478 | 1.00 | 160.87 |
| 7349 | OG1 | THR | E | 152 | −23.649 | 67.242 | −25.840 | 1.00 | 160.87 |
| 7350 | CG2 | THR | E | 152 | −22.443 | 65.158 | −25.963 | 1.00 | 160.87 |
| 7351 | C | THR | E | 152 | −21.530 | 68.832 | −26.420 | 1.00 | 109.73 |
| 7352 | O | THR | E | 152 | −21.983 | 69.541 | −25.509 | 1.00 | 109.73 |
| 7353 | N | GLY | E | 153 | −21.284 | 69.305 | −27.640 | 1.00 | 146.21 |
| 7354 | CA | GLY | E | 153 | −21.530 | 70.706 | −27.927 | 1.00 | 146.21 |
| 7355 | C | GLY | E | 153 | −21.486 | 71.046 | −29.398 | 1.00 | 146.21 |
| 7356 | O | GLY | E | 153 | −21.040 | 70.244 | −30.204 | 1.00 | 146.21 |
| 7357 | N | LYS | E | 154 | −21.947 | 72.247 | −29.739 | 1.00 | 118.16 |
| 7358 | CA | LYS | E | 154 | −21.973 | 72.704 | −31.114 | 1.00 | 118.16 |
| 7359 | CB | LYS | E | 154 | −23.332 | 73.316 | −31.423 | 1.00 | 235.84 |
| 7360 | CG | LYS | E | 154 | −23.517 | 73.732 | −32.861 | 1.00 | 235.84 |
| 7361 | CD | LYS | E | 154 | −24.925 | 74.260 | −33.087 | 1.00 | 235.84 |
| 7362 | CE | LYS | E | 154 | −25.120 | 74.744 | −34.517 | 1.00 | 235.84 |
| 7363 | NZ | LYS | E | 154 | −26.493 | 75.280 | −34.746 | 1.00 | 235.84 |
| 7364 | C | LYS | E | 154 | −20.864 | 73.716 | −31.376 | 1.00 | 118.16 |
| 7365 | O | LYS | E | 154 | −20.821 | 74.780 | −30.758 | 1.00 | 118.16 |
| 7366 | N | VAL | E | 155 | −19.957 | 73.364 | −32.288 | 1.00 | 164.34 |
| 7367 | CA | VAL | E | 155 | −18.825 | 74.214 | −32.686 | 1.00 | 164.34 |
| 7368 | CB | VAL | E | 155 | −17.520 | 73.384 | −32.768 | 1.00 | 138.01 |
| 7369 | CG1 | VAL | E | 155 | −16.369 | 74.233 | −33.254 | 1.00 | 138.01 |
| 7370 | CG2 | VAL | E | 155 | −17.198 | 72.808 | −31.398 | 1.00 | 138.01 |
| 7371 | C | VAL | E | 155 | −19.166 | 74.746 | −34.073 | 1.00 | 164.34 |
| 7372 | O | VAL | E | 155 | −19.503 | 73.962 | −34.965 | 1.00 | 164.34 |
| 7373 | N | TRP | E | 156 | −19.058 | 76.060 | −34.268 | 1.00 | 249.37 |
| 7374 | CA | TRP | E | 156 | −19.413 | 76.665 | −35.557 | 1.00 | 249.37 |
| 7375 | CB | TRP | E | 156 | −18.639 | 76.057 | −36.746 | 1.00 | 249.69 |
| 7376 | CG | TRP | E | 156 | −17.160 | 76.335 | −36.808 | 1.00 | 249.69 |
| 7377 | CD2 | TRP | E | 156 | −16.521 | 77.599 | −37.045 | 1.00 | 249.69 |
| 7378 | CE2 | TRP | E | 156 | −15.126 | 77.369 | −37.030 | 1.00 | 249.69 |
| 7379 | CE3 | TRP | E | 156 | −16.992 | 78.898 | −37.283 | 1.00 | 249.69 |
| 7380 | CD1 | TRP | E | 156 | −16.152 | 75.425 | −36.653 | 1.00 | 249.69 |
| 7381 | NE1 | TRP | E | 156 | −14.929 | 76.035 | −36.786 | 1.00 | 249.69 |
| 7382 | CZ2 | TRP | E | 156 | −14.197 | 78.389 | −37.233 | 1.00 | 249.69 |
| 7383 | CZ3 | TRP | E | 156 | −16.067 | 79.915 | −37.486 | 1.00 | 249.69 |
| 7384 | CH2 | TRP | E | 156 | −14.684 | 79.652 | −37.459 | 1.00 | 249.69 |
| 7385 | C | TRP | E | 156 | −20.881 | 76.332 | −35.750 | 1.00 | 249.37 |
| 7386 | O | TRP | E | 156 | −21.762 | 77.001 | −35.194 | 1.00 | 249.37 |
| 7387 | N | GLN | E | 157 | −21.128 | 75.279 | −36.536 | 1.00 | 132.72 |
| 7388 | CA | GLN | E | 157 | −22.489 | 74.837 | −36.802 | 1.00 | 132.72 |
| 7389 | CB | GLN | E | 157 | −23.006 | 75.460 | −38.103 | 1.00 | 249.69 |
| 7390 | CG | GLN | E | 157 | −23.387 | 76.941 | −37.974 | 1.00 | 249.69 |
| 7391 | CD | GLN | E | 157 | −24.572 | 77.181 | −37.037 | 1.00 | 249.69 |
| 7392 | OE1 | GLN | E | 157 | −25.685 | 76.713 | −37.291 | 1.00 | 249.69 |
| 7393 | NE2 | GLN | E | 157 | −24.334 | 77.914 | −35.949 | 1.00 | 249.69 |
| 7394 | C | GLN | E | 157 | −22.668 | 73.317 | −36.834 | 1.00 | 132.72 |
| 7395 | O | GLN | E | 157 | −23.628 | 72.812 | −37.423 | 1.00 | 132.72 |
| 7396 | N | LEU | E | 158 | −21.756 | 72.587 | −36.195 | 1.00 | 229.55 |
| 7397 | CA | LEU | E | 158 | −21.863 | 71.130 | −36.137 | 1.00 | 229.55 |
| 7398 | CB | LEU | E | 158 | −20.818 | 70.467 | −37.038 | 1.00 | 228.12 |
| 7399 | CG | LEU | E | 158 | −21.063 | 70.461 | −38.553 | 1.00 | 228.12 |
| 7400 | CD1 | LEU | E | 158 | −20.552 | 69.140 | −39.108 | 1.00 | 228.12 |
| 7401 | CD2 | LEU | E | 158 | −22.544 | 70.590 | −38.874 | 1.00 | 228.12 |
| 7402 | C | LEU | E | 158 | −21.716 | 70.605 | −34.713 | 1.00 | 229.55 |
| 7403 | O | LEU | E | 158 | −21.041 | 71.216 | −33.885 | 1.00 | 229.55 |
| 7404 | N | ASP | E | 159 | −22.357 | 69.472 | −34.438 | 1.00 | 199.14 |
| 7405 | CA | ASP | E | 159 | −22.299 | 68.862 | −33.114 | 1.00 | 199.14 |
| 7406 | CB | ASP | E | 159 | −23.567 | 68.050 | −32.848 | 1.00 | 198.75 |
| 7407 | CG | ASP | E | 159 | −24.829 | 68.854 | −33.075 | 1.00 | 198.75 |
| 7408 | OD1 | ASP | E | 159 | −25.033 | 69.866 | −32.365 | 1.00 | 198.75 |
| 7409 | OD2 | ASP | E | 159 | −25.613 | 68.475 | −33.970 | 1.00 | 198.75 |
| 7410 | C | ASP | E | 159 | −21.082 | 67.948 | −32.988 | 1.00 | 199.14 |
| 7411 | O | ASP | E | 159 | −20.656 | 67.333 | −33.963 | 1.00 | 199.14 |
| 7412 | N | TYR | E | 160 | −20.522 | 67.867 | −31.784 | 1.00 | 164.98 |
| 7413 | CA | TYR | E | 160 | −19.368 | 67.017 | −31.543 | 1.00 | 164.98 |
| 7414 | CB | TYR | E | 160 | −18.071 | 67.782 | −31.730 | 1.00 | 170.02 |
| 7415 | CG | TYR | E | 160 | −17.959 | 68.445 | −33.079 | 1.00 | 170.02 |
| 7416 | CD1 | TYR | E | 160 | −18.428 | 69.746 | −33.285 | 1.00 | 170.02 |
| 7417 | CE1 | TYR | E | 160 | −18.291 | 70.376 | −34.516 | 1.00 | 170.02 |
| 7418 | CD2 | TYR | E | 160 | −17.362 | 67.787 | −34.147 | 1.00 | 170.02 |
| 7419 | CE2 | TYR | E | 160 | −17.221 | 68.407 | −35.390 | 1.00 | 170.02 |
| 7420 | CZ | TYR | E | 160 | −17.685 | 69.700 | −35.562 | 1.00 | 170.02 |
| 7421 | OH | TYR | E | 160 | −17.517 | 70.325 | −36.771 | 1.00 | 170.02 |
| 7422 | C | TYR | E | 160 | −19.385 | 66.416 | −30.153 | 1.00 | 164.98 |
| 7423 | O | TYR | E | 160 | −19.844 | 67.025 | −29.185 | 1.00 | 164.98 |
| 7424 | N | GLU | E | 161 | −18.861 | 65.204 | −30.073 | 1.00 | 121.76 |
| 7425 | CA | GLU | E | 161 | −18.805 | 64.435 | −28.835 | 1.00 | 121.76 |
| 7426 | CB | GLU | E | 161 | −19.432 | 63.056 | −29.104 | 1.00 | 238.30 |
| 7427 | CG | GLU | E | 161 | −19.437 | 62.062 | −27.962 | 1.00 | 238.30 |
| 7428 | CD | GLU | E | 161 | −20.385 | 60.898 | −28.227 | 1.00 | 238.30 |
| 7429 | OE1 | GLU | E | 161 | −20.217 | 59.834 | −27.596 | 1.00 | 238.30 |
| 7430 | OE2 | GLU | E | 161 | −21.310 | 61.051 | −29.058 | 1.00 | 238.30 |
| 7431 | C | GLU | E | 161 | −17.340 | 64.330 | −28.425 | 1.00 | 121.76 |
| 7432 | O | GLU | E | 161 | −16.464 | 64.228 | −29.275 | 1.00 | 121.76 |
| 7433 | N | SER | E | 162 | −17.079 | 64.384 | −27.125 | 1.00 | 141.30 |
| 7434 | CA | SER | E | 162 | −15.712 | 64.316 | −26.603 | 1.00 | 141.30 |
| 7435 | CB | SER | E | 162 | −15.579 | 65.188 | −25.350 | 1.00 | 137.77 |
| 7436 | OG | SER | E | 162 | −16.423 | 64.719 | −24.305 | 1.00 | 137.77 |
| 7437 | C | SER | E | 162 | −15.318 | 62.905 | −26.240 | 1.00 | 141.30 |
| 7438 | O | SER | E | 162 | −16.181 | 62.046 | −26.067 | 1.00 | 141.30 |
| 7439 | N | GLU | E | 163 | −14.015 | 62.662 | −26.132 | 1.00 | 137.45 |
| 7440 | CA | GLU | E | 163 | −13.553 | 61.335 | −25.739 | 1.00 | 137.45 |
| 7441 | CB | GLU | E | 163 | −12.021 | 61.264 | −25.770 | 1.00 | 249.69 |
| 7442 | CG | GLU | E | 163 | −11.400 | 61.173 | −27.169 | 1.00 | 249.69 |
| 7443 | CD | GLU | E | 163 | −11.585 | 59.804 | −27.826 | 1.00 | 249.69 |
| 7444 | OE1 | GLU | E | 163 | −11.171 | 58.791 | −27.226 | 1.00 | 249.69 |
| 7445 | OE2 | GLU | E | 163 | −12.138 | 59.742 | −28.944 | 1.00 | 249.69 |
| 7446 | C | GLU | E | 163 | −14.057 | 61.170 | −24.309 | 1.00 | 137.45 |
| 7447 | O | GLU | E | 163 | −14.182 | 62.171 | −23.593 | 1.00 | 137.45 |
| 7448 | N | PRO | E | 164 | −14.377 | 59.935 | −23.877 | 1.00 | 95.03 |
| 7449 | CD | PRO | E | 164 | −14.382 | 58.681 | −24.662 | 1.00 | 218.77 |
| 7450 | CA | PRO | E | 164 | −14.877 | 59.684 | −22.521 | 1.00 | 95.03 |
| 7451 | CB | PRO | E | 164 | −15.570 | 58.345 | −22.657 | 1.00 | 218.77 |
| 7452 | CG | PRO | E | 164 | −14.657 | 57.637 | −23.594 | 1.00 | 218.77 |
| 7453 | C | PRO | E | 164 | −13.761 | 59.664 | −21.475 | 1.00 | 95.03 |
| 7454 | O | PRO | E | 164 | −12.660 | 59.202 | −21.764 | 1.00 | 95.03 |
| 7455 | N | LEU | E | 165 | −14.046 | 60.133 | −20.267 | 1.00 | 132.61 |
| 7456 | CA | LEU | E | 165 | −13.021 | 60.175 | −19.240 | 1.00 | 132.61 |
| 7457 | CB | LEU | E | 165 | −12.581 | 61.627 | −19.014 | 1.00 | 87.30 |
| 7458 | CG | LEU | E | 165 | −11.475 | 61.851 | −17.979 | 1.00 | 87.30 |
| 7459 | CD1 | LEU | E | 165 | −10.446 | 60.707 | −18.043 | 1.00 | 87.30 |
| 7460 | CD2 | LEU | E | 165 | −10.812 | 63.181 | −18.220 | 1.00 | 87.30 |
| 7461 | C | LEU | E | 165 | −13.411 | 59.560 | −17.905 | 1.00 | 132.61 |
| 7462 | O | LEU | E | 165 | −14.470 | 59.883 | −17.367 | 1.00 | 132.61 |
| 7463 | N | ASN | E | 166 | −12.545 | 58.690 | −17.364 | 1.00 | 112.66 |
| 7464 | CA | ASN | E | 166 | −12.794 | 58.050 | −16.068 | 1.00 | 112.66 |
| 7465 | CB | ASN | E | 166 | −12.116 | 56.693 | −16.002 | 1.00 | 172.55 |
| 7466 | CG | ASN | E | 166 | −13.038 | 55.560 | −16.402 | 1.00 | 172.55 |
| 7467 | OD1 | ASN | E | 166 | −14.264 | 55.679 | −16.328 | 1.00 | 172.55 |
| 7468 | ND2 | ASN | E | 166 | −12.445 | 54.443 | −16.804 | 1.00 | 172.55 |
| 7469 | C | ASN | E | 166 | −12.294 | 58.889 | −14.909 | 1.00 | 112.66 |
| 7470 | O | ASN | E | 166 | −11.246 | 59.511 | −14.999 | 1.00 | 112.66 |
| 7471 | N | ILE | E | 167 | −13.032 | 58.887 | −13.807 | 1.00 | 147.51 |
| 7472 | CA | ILE | E | 167 | −12.643 | 59.658 | −12.628 | 1.00 | 147.51 |
| 7473 | CB | ILE | E | 167 | −13.409 | 60.966 | −12.546 | 1.00 | 109.60 |
| 7474 | CG2 | ILE | E | 167 | −13.051 | 61.688 | −11.260 | 1.00 | 109.60 |
| 7475 | CG1 | ILE | E | 167 | −13.086 | 61.820 | −13.760 | 1.00 | 109.60 |
| 7476 | CD1 | ILE | E | 167 | −13.847 | 63.089 | −13.806 | 1.00 | 109.60 |
| 7477 | C | ILE | E | 167 | −12.904 | 58.901 | −11.343 | 1.00 | 147.51 |
| 7478 | O | ILE | E | 167 | −14.007 | 58.401 | −11.115 | 1.00 | 147.51 |
| 7479 | N | THR | E | 168 | −11.903 | 58.840 | −10.481 | 1.00 | 104.75 |
| 7480 | CA | THR | E | 168 | −12.093 | 58.106 | −9.251 | 1.00 | 104.75 |
| 7481 | CB | THR | E | 168 | −11.250 | 56.819 | −9.263 | 1.00 | 148.63 |
| 7482 | OG1 | THR | E | 168 | −11.607 | 56.040 | −10.408 | 1.00 | 148.63 |
| 7483 | CG2 | THR | E | 168 | −11.516 | 55.997 | −8.014 | 1.00 | 148.63 |
| 7484 | C | THR | E | 168 | −11.831 | 58.886 | −7.956 | 1.00 | 104.75 |
| 7485 | O | THR | E | 168 | −10.763 | 59.432 | −7.718 | 1.00 | 104.75 |
| 7486 | N | VAL | E | 169 | −12.849 | 58.924 | −7.117 | 1.00 | 128.03 |
| 7487 | CA | VAL | E | 169 | −12.771 | 59.592 | −5.835 | 1.00 | 128.03 |
| 7488 | CB | VAL | E | 169 | −14.028 | 60.478 | −5.592 | 1.00 | 104.73 |
| 7489 | CG1 | VAL | E | 169 | −14.231 | 60.741 | −4.125 | 1.00 | 104.73 |
| 7490 | CG2 | VAL | E | 169 | −13.853 | 61.798 | −6.296 | 1.00 | 104.73 |
| 7491 | C | VAL | E | 169 | −12.683 | 58.492 | −4.783 | 1.00 | 128.03 |
| 7492 | O | VAL | E | 169 | −13.645 | 57.767 | −4.542 | 1.00 | 128.03 |
| 7493 | N | ILE | E | 170 | −11.510 | 58.363 | −4.171 | 1.00 | 164.43 |
| 7494 | CA | ILE | E | 170 | −11.265 | 57.356 | −3.135 | 1.00 | 164.43 |
| 7495 | CB | ILE | E | 170 | −9.826 | 56.804 | −3.258 | 1.00 | 148.98 |
| 7496 | CG2 | ILE | E | 170 | −9.566 | 56.357 | −4.685 | 1.00 | 148.98 |
| 7497 | CG1 | ILE | E | 170 | −8.816 | 57.897 | −2.906 | 1.00 | 148.98 |
| 7498 | CD1 | ILE | E | 170 | −7.368 | 57.440 | −2.961 | 1.00 | 148.98 |
| 7499 | C | ILE | E | 170 | −11.467 | 57.968 | −1.744 | 1.00 | 164.43 |
| 7500 | O | ILE | E | 170 | −11.524 | 59.184 | −1.610 | 1.00 | 164.43 |
| 7501 | N | LYS | E | 171 | −11.560 | 57.141 | −0.709 | 1.00 | 181.58 |
| 7502 | CA | LYS | E | 171 | −11.775 | 57.672 | 0.633 | 1.00 | 181.58 |
| 7503 | CB | LYS | E | 171 | −13.118 | 57.176 | 1.157 | 1.00 | 249.69 |
| 7504 | CG | LYS | E | 171 | −13.230 | 55.666 | 1.123 | 1.00 | 249.69 |
| 7505 | CD | LYS | E | 171 | −14.675 | 55.211 | 0.996 | 1.00 | 249.69 |
| 7506 | CE | LYS | E | 171 | −15.530 | 55.712 | 2.154 | 1.00 | 249.69 |
| 7507 | NZ | LYS | E | 171 | −16.931 | 55.206 | 2.058 | 1.00 | 249.69 |
| 7508 | C | LYS | E | 171 | −10.673 | 57.327 | 1.632 | 1.00 | 181.58 |
| 7509 | O | LYS | E | 171 | −10.810 | 57.585 | 2.833 | 1.00 | 181.58 |
| 7510 | C1 | NAG | E | 221 | 2.209 | 79.546 | −26.386 | 1.00 | 249.69 |
| 7511 | C2 | NAG | E | 221 | 0.889 | 80.273 | −26.643 | 1.00 | 249.69 |
| 7512 | N2 | NAG | E | 221 | −0.170 | 79.298 | −26.828 | 1.00 | 249.69 |
| 7513 | C7 | NAG | E | 221 | −1.431 | 79.634 | −26.596 | 1.00 | 249.69 |
| 7514 | O7 | NAG | E | 221 | −1.768 | 80.762 | −26.235 | 1.00 | 249.69 |
| 7515 | C8 | NAG | E | 221 | −2.476 | 78.553 | −26.806 | 1.00 | 249.69 |
| 7516 | C3 | NAG | E | 221 | 1.001 | 81.165 | −27.881 | 1.00 | 249.69 |
| 7517 | O3 | NAG | E | 221 | −0.178 | 81.953 | −28.009 | 1.00 | 249.69 |
| 7518 | C4 | NAG | E | 221 | 2.237 | 82.087 | −27.816 | 1.00 | 249.69 |
| 7519 | O4 | NAG | E | 221 | 2.396 | 82.690 | −29.116 | 1.00 | 249.69 |
| 7520 | C5 | NAG | E | 221 | 3.502 | 81.275 | −27.447 | 1.00 | 249.69 |
| 7521 | O5 | NAG | E | 221 | 3.276 | 80.502 | −26.244 | 1.00 | 249.69 |
| 7522 | C6 | NAG | E | 221 | 4.726 | 82.136 | −27.185 | 1.00 | 249.69 |
| 7523 | O6 | NAG | E | 221 | 4.477 | 83.098 | −26.171 | 1.00 | 249.69 |
| 7524 | C1 | NAG | E | 222 | 3.181 | 83.831 | −29.256 | 1.00 | 249.69 |
| 7525 | C2 | NAG | E | 222 | 2.456 | 84.839 | −30.180 | 1.00 | 249.69 |
| 7526 | N2 | NAG | E | 222 | 1.186 | 85.239 | −29.587 | 1.00 | 249.69 |
| 7527 | C7 | NAG | E | 222 | 0.936 | 86.518 | −29.301 | 1.00 | 249.69 |
| 7528 | O7 | NAG | E | 222 | 1.741 | 87.429 | −29.516 | 1.00 | 249.69 |
| 7529 | C8 | NAG | E | 222 | −0.420 | 86.835 | −28.681 | 1.00 | 249.69 |
| 7530 | C3 | NAG | E | 222 | 2.220 | 84.191 | −31.568 | 1.00 | 249.69 |
| 7531 | O3 | NAG | E | 222 | 1.662 | 85.147 | −32.469 | 1.00 | 249.69 |
| 7532 | C4 | NAG | E | 222 | 3.543 | 83.638 | −32.143 | 1.00 | 249.69 |
| 7533 | O4 | NAG | E | 222 | 3.281 | 82.909 | −33.338 | 1.00 | 249.69 |
| 7534 | C5 | NAG | E | 222 | 4.233 | 82.718 | −31.115 | 1.00 | 249.69 |
| 7535 | O5 | NAG | E | 222 | 4.427 | 83.426 | −29.859 | 1.00 | 249.69 |
| 7536 | C6 | NAG | E | 222 | 5.592 | 82.211 | −31.572 | 1.00 | 249.69 |
| 7537 | O6 | NAG | E | 222 | 5.701 | 80.806 | −31.409 | 1.00 | 249.69 |
| 7538 | C1 | NAG | E | 242 | 7.147 | 59.017 | −23.850 | 1.00 | 193.96 |
| 7539 | C2 | NAG | E | 242 | 7.463 | 59.646 | −25.212 | 1.00 | 193.96 |
| 7540 | N2 | NAG | E | 242 | 8.286 | 60.830 | −25.064 | 1.00 | 193.96 |
| 7541 | C7 | NAG | E | 242 | 9.478 | 60.868 | −25.645 | 1.00 | 193.96 |
| 7542 | O7 | NAG | E | 242 | 9.927 | 59.930 | −26.305 | 1.00 | 193.96 |
| 7543 | C8 | NAG | E | 242 | 10.299 | 62.130 | −25.465 | 1.00 | 193.96 |
| 7544 | C3 | NAG | E | 242 | 6.151 | 59.995 | −25.913 | 1.00 | 193.96 |
| 7545 | O3 | NAG | E | 242 | 6.418 | 60.545 | −27.194 | 1.00 | 193.96 |
| 7546 | C4 | NAG | E | 242 | 5.284 | 58.740 | −26.060 | 1.00 | 193.96 |
| 7547 | O4 | NAG | E | 242 | 3.983 | 59.116 | −26.566 | 1.00 | 193.96 |
| 7548 | C5 | NAG | E | 242 | 5.124 | 58.005 | −24.698 | 1.00 | 193.96 |
| 7549 | O5 | NAG | E | 242 | 6.411 | 57.799 | −24.050 | 1.00 | 193.96 |
| 7550 | C6 | NAG | E | 242 | 4.509 | 56.624 | −24.872 | 1.00 | 193.96 |
| 7551 | O6 | NAG | E | 242 | 3.211 | 56.550 | −24.304 | 1.00 | 193.96 |
| 7552 | C1 | NAG | E | 243 | 3.598 | 58.568 | −27.770 | 1.00 | 215.12 |
| 7553 | C2 | NAG | E | 243 | 2.085 | 58.638 | −27.907 | 1.00 | 215.12 |
| 7554 | N2 | NAG | E | 243 | 1.433 | 57.909 | −26.843 | 1.00 | 215.12 |
| 7555 | C7 | NAG | E | 243 | 0.428 | 58.482 | −26.182 | 1.00 | 215.12 |
| 7556 | O7 | NAG | E | 243 | 0.027 | 59.633 | −26.423 | 1.00 | 215.12 |
| 7557 | C8 | NAG | E | 243 | −0.230 | 57.665 | −25.075 | 1.00 | 215.12 |
| 7558 | C3 | NAG | E | 243 | 1.685 | 58.056 | −29.247 | 1.00 | 215.12 |
| 7559 | O3 | NAG | E | 243 | 0.272 | 58.105 | −29.401 | 1.00 | 215.12 |
| 7560 | C4 | NAG | E | 243 | 2.344 | 58.866 | −30.339 | 1.00 | 215.12 |
| 7561 | O4 | NAG | E | 243 | 1.898 | 58.318 | −31.574 | 1.00 | 215.12 |
| 7562 | C5 | NAG | E | 243 | 3.883 | 58.823 | −30.140 | 1.00 | 215.12 |
| 7563 | O5 | NAG | E | 243 | 4.208 | 59.328 | −28.814 | 1.00 | 215.12 |
| 7564 | C6 | NAG | E | 243 | 4.624 | 59.699 | −31.116 | 1.00 | 215.12 |
| 7565 | O6 | NAG | E | 243 | 4.268 | 61.057 | −30.933 | 1.00 | 215.12 |
| 7566 | C1 | MAN | E | 244 | 1.748 | 59.080 | −32.701 | 1.00 | 219.74 |
| 7567 | C2 | MAN | E | 244 | 2.233 | 58.170 | −33.738 | 1.00 | 219.74 |
| 7568 | O2 | MAN | E | 244 | 1.708 | 56.848 | −33.490 | 1.00 | 219.74 |
| 7569 | C3 | MAN | E | 244 | 1.963 | 58.748 | −35.107 | 1.00 | 219.74 |
| 7570 | O3 | MAN | E | 244 | 2.548 | 57.949 | −36.119 | 1.00 | 219.74 |
| 7571 | C4 | MAN | E | 244 | 0.488 | 58.983 | −35.314 | 1.00 | 219.74 |
| 7572 | O4 | MAN | E | 244 | 0.264 | 59.475 | −36.620 | 1.00 | 219.74 |
| 7573 | C5 | MAN | E | 244 | 0.038 | 59.992 | −34.253 | 1.00 | 219.74 |
| 7574 | O5 | MAN | E | 244 | 0.282 | 59.411 | −32.908 | 1.00 | 219.74 |
| 7575 | C6 | MAN | E | 244 | −1.419 | 60.489 | −34.434 | 1.00 | 219.74 |
| 7576 | O6 | MAN | E | 244 | −2.389 | 59.610 | −33.877 | 1.00 | 219.74 |
| 7577 | C1 | NAG | E | 250 | 12.894 | 79.616 | −14.981 | 1.00 | 249.69 |
| 7578 | C2 | NAG | E | 250 | 12.331 | 80.923 | −14.392 | 1.00 | 249.69 |
| 7579 | N2 | NAG | E | 250 | 12.256 | 80.832 | −12.946 | 1.00 | 249.69 |
| 7580 | C7 | NAG | E | 250 | 13.100 | 81.532 | −12.196 | 1.00 | 249.69 |
| 7581 | O7 | NAG | E | 250 | 13.967 | 82.276 | −12.673 | 1.00 | 249.69 |
| 7582 | C8 | NAG | E | 250 | 12.966 | 81.387 | −10.683 | 1.00 | 249.69 |
| 7583 | C3 | NAG | E | 250 | 10.934 | 81.188 | −14.970 | 1.00 | 249.69 |
| 7584 | O3 | NAG | E | 250 | 10.442 | 82.440 | −14.506 | 1.00 | 249.69 |
| 7585 | C4 | NAG | E | 250 | 10.987 | 81.183 | −16.508 | 1.00 | 249.69 |
| 7586 | O4 | NAG | E | 250 | 9.667 | 81.305 | −17.032 | 1.00 | 249.69 |
| 7587 | C5 | NAG | E | 250 | 11.643 | 79.872 | −17.010 | 1.00 | 249.69 |
| 7588 | O5 | NAG | E | 250 | 12.954 | 79.705 | −16.412 | 1.00 | 249.69 |
| 7589 | C6 | NAG | E | 250 | 11.833 | 79.816 | −18.522 | 1.00 | 249.69 |
| 7590 | O6 | NAG | E | 250 | 12.752 | 78.791 | −18.892 | 1.00 | 249.69 |
| 7591 | C1 | NAG | E | 274 | 14.635 | 58.650 | 0.211 | 1.00 | 249.69 |
| 7592 | C2 | NAG | E | 274 | 13.525 | 58.145 | 1.158 | 1.00 | 249.69 |
| 7593 | N2 | NAG | E | 274 | 13.058 | 59.230 | 2.009 | 1.00 | 249.69 |
| 7594 | C7 | NAG | E | 274 | 11.826 | 59.208 | 2.513 | 1.00 | 249.69 |
| 7595 | O7 | NAG | E | 274 | 11.030 | 58.289 | 2.302 | 1.00 | 249.69 |
| 7596 | C8 | NAG | E | 274 | 11.415 | 60.380 | 3.387 | 1.00 | 249.69 |
| 7597 | C3 | NAG | E | 274 | 14.058 | 56.984 | 2.020 | 1.00 | 249.69 |
| 7598 | O3 | NAG | E | 274 | 12.997 | 56.422 | 2.785 | 1.00 | 249.69 |
| 7599 | C4 | NAG | E | 274 | 14.687 | 55.894 | 1.134 | 1.00 | 249.69 |
| 7600 | O4 | NAG | E | 274 | 15.298 | 54.900 | 1.951 | 1.00 | 249.69 |
| 7601 | C5 | NAG | E | 274 | 15.736 | 56.513 | 0.196 | 1.00 | 249.69 |
| 7602 | O5 | NAG | E | 274 | 15.136 | 57.567 | −0.595 | 1.00 | 249.69 |
| 7603 | C6 | NAG | E | 274 | 16.324 | 55.500 | −0.775 | 1.00 | 249.69 |
| 7604 | O6 | NAG | E | 274 | 17.151 | 56.129 | −1.748 | 1.00 | 249.69 |
| 7605 | C1 | NAG | E | 335 | −13.218 | 77.155 | −18.184 | 1.00 | 248.99 |
| 7606 | C2 | NAG | E | 335 | −12.377 | 77.952 | −17.147 | 1.00 | 248.99 |
| 7607 | N2 | NAG | E | 335 | −13.025 | 77.859 | −15.850 | 1.00 | 248.99 |
| 7608 | C7 | NAG | E | 335 | −12.415 | 77.253 | −14.835 | 1.00 | 248.99 |
| 7609 | O7 | NAG | E | 335 | −11.291 | 76.751 | −14.921 | 1.00 | 248.99 |
| 7610 | C8 | NAG | E | 335 | −13.169 | 77.199 | −13.517 | 1.00 | 248.99 |
| 7611 | C3 | NAG | E | 335 | −12.169 | 79.444 | −17.498 | 1.00 | 248.99 |
| 7612 | O3 | NAG | E | 335 | −11.051 | 79.949 | −16.774 | 1.00 | 248.99 |
| 7613 | C4 | NAG | E | 335 | −11.918 | 79.636 | −18.990 | 1.00 | 248.99 |
| 7614 | O4 | NAG | E | 335 | −11.812 | 81.021 | −19.294 | 1.00 | 248.99 |
| 7615 | C5 | NAG | E | 335 | −13.079 | 79.014 | −19.748 | 1.00 | 248.99 |
| 7616 | O5 | NAG | E | 335 | −13.060 | 77.584 | −19.562 | 1.00 | 248.99 |
| 7617 | C6 | NAG | E | 335 | −12.991 | 79.270 | −21.238 | 1.00 | 248.99 |
| 7618 | O6 | NAG | E | 335 | −14.176 | 79.882 | −21.722 | 1.00 | 248.99 |
| 7619 | C1 | NAG | E | 340 | −18.408 | 67.970 | 2.712 | 1.00 | 249.69 |
| 7620 | C2 | NAG | E | 340 | −17.972 | 66.798 | 3.606 | 1.00 | 249.69 |
| 7621 | N2 | NAG | E | 340 | −17.526 | 65.688 | 2.783 | 1.00 | 249.69 |
| 7622 | C7 | NAG | E | 340 | −16.380 | 65.065 | 3.055 | 1.00 | 249.69 |
| 7623 | O7 | NAG | E | 340 | −15.644 | 65.372 | 4.003 | 1.00 | 249.69 |
| 7624 | C8 | NAG | E | 340 | −15.987 | 63.917 | 2.140 | 1.00 | 249.69 |
| 7625 | C3 | NAG | E | 340 | −19.162 | 66.374 | 4.487 | 1.00 | 249.69 |
| 7626 | O3 | NAG | E | 340 | −18.769 | 65.355 | 5.395 | 1.00 | 249.69 |
| 7627 | C4 | NAG | E | 340 | −19.704 | 67.580 | 5.273 | 1.00 | 249.69 |
| 7628 | O4 | NAG | E | 340 | −20.884 | 67.199 | 5.975 | 1.00 | 249.69 |
| 7629 | C5 | NAG | E | 340 | −20.011 | 68.748 | 4.305 | 1.00 | 249.69 |
| 7630 | O5 | NAG | E | 340 | −18.836 | 69.073 | 3.520 | 1.00 | 249.69 |
| 7631 | C6 | NAG | E | 340 | −20.450 | 70.028 | 5.000 | 1.00 | 249.69 |
| 7632 | O6 | NAG | E | 340 | −20.520 | 71.112 | 4.081 | 1.00 | 249.69 |
| 7633 | C1 | NAG | E | 366 | −13.236 | 53.354 | −17.338 | 1.00 | 200.99 |
| 7634 | C2 | NAG | E | 366 | −12.501 | 52.697 | −18.503 | 1.00 | 200.99 |
| 7635 | N2 | NAG | E | 366 | −12.267 | 53.689 | −19.539 | 1.00 | 200.99 |
| 7636 | C7 | NAG | E | 366 | −11.142 | 54.405 | −19.544 | 1.00 | 200.99 |
| 7637 | O7 | NAG | E | 366 | −10.251 | 54.258 | −18.697 | 1.00 | 200.99 |
| 7638 | C8 | NAG | E | 366 | −10.974 | 55.435 | −20.659 | 1.00 | 200.99 |
| 7639 | C3 | NAG | E | 366 | −13.344 | 51.559 | −19.064 | 1.00 | 200.99 |
| 7640 | O3 | NAG | E | 366 | −12.589 | 50.845 | −20.024 | 1.00 | 200.99 |
| 7641 | C4 | NAG | E | 366 | −13.814 | 50.601 | −17.968 | 1.00 | 200.99 |
| 7642 | O4 | NAG | E | 366 | −14.809 | 49.714 | −18.523 | 1.00 | 200.99 |
| 7643 | C5 | NAG | E | 366 | −14.427 | 51.387 | −16.796 | 1.00 | 200.99 |
| 7644 | O5 | NAG | E | 366 | −13.511 | 52.389 | −16.333 | 1.00 | 200.99 |
| 7645 | C6 | NAG | E | 366 | −14.780 | 50.532 | −15.594 | 1.00 | 200.99 |
| 7646 | O6 | NAG | E | 366 | −15.500 | 51.287 | −14.628 | 1.00 | 200.99 |
| 7647 | C1 | NAG | E | 367 | −14.595 | 48.351 | −18.366 | 1.00 | 248.88 |
| 7648 | C2 | NAG | E | 367 | −15.915 | 47.598 | −18.528 | 1.00 | 248.88 |
| 7649 | N2 | NAG | E | 367 | −16.897 | 48.084 | −17.575 | 1.00 | 248.88 |
| 7650 | C7 | NAG | E | 367 | −17.964 | 48.748 | −18.004 | 1.00 | 248.88 |
| 7651 | O7 | NAG | E | 367 | −18.175 | 48.977 | −19.195 | 1.00 | 248.88 |
| 7652 | C8 | NAG | E | 367 | −18.948 | 49.229 | −16.950 | 1.00 | 248.88 |
| 7653 | C3 | NAG | E | 367 | −15.646 | 46.102 | −18.325 | 1.00 | 248.88 |
| 7654 | O3 | NAG | E | 367 | −16.851 | 45.362 | −18.485 | 1.00 | 248.88 |
| 7655 | C4 | NAG | E | 367 | −14.602 | 45.631 | −19.346 | 1.00 | 248.88 |
| 7656 | O4 | NAG | E | 367 | −14.273 | 44.271 | −19.099 | 1.00 | 248.88 |
| 7657 | C5 | NAG | E | 367 | −13.334 | 46.502 | −19.256 | 1.00 | 248.88 |
| 7658 | O5 | NAG | E | 367 | −13.668 | 47.911 | −19.373 | 1.00 | 248.88 |
| 7659 | C6 | NAG | E | 367 | −12.347 | 46.188 | −20.363 | 1.00 | 248.88 |
| 7660 | O6 | NAG | E | 367 | −12.226 | 47.271 | −21.276 | 1.00 | 248.88 |

TABLE 7
Atomic coordinates of PhFcεRIα₁₋₁₇₆, Form M2

| ATOM | ATOM | | | | | | | |
|---|---|---|---|---|---|---|---|---|
| NUMBER | TYPE | RESIDUE | # | X | Y | Z | occ | B |
| 1 | CB | VAL A | 1 | 54.132 | −20.714 | 8.499 | 1.00 | 178.10 |
| 2 | CG1 | VAL A | 1 | 52.843 | −21.062 | 7.774 | 1.00 | 175.86 |
| 3 | CG2 | VAL A | 1 | 54.598 | −21.899 | 9.342 | 1.00 | 170.07 |
| 4 | C | VAL A | 1 | 55.044 | −18.854 | 6.922 | 1.00 | 182.13 |
| 5 | O | VAL A | 1 | 54.219 | −18.626 | 6.045 | 1.00 | 181.45 |
| 6 | N | VAL A | 1 | 56.560 | −20.445 | 8.067 | 1.00 | 185.40 |
| 7 | CA | VAL A | 1 | 55.237 | −20.291 | 7.470 | 1.00 | 181.27 |
| 8 | N | PRO A | 2 | 55.807 | −17.881 | 7.435 | 1.00 | 180.05 |
| 9 | CD | PRO A | 2 | 55.929 | −17.840 | 8.889 | 1.00 | 177.25 |
| 10 | CA | PRO A | 2 | 55.680 | −16.493 | 6.950 | 1.00 | 173.64 |
| 11 | CB | PRO A | 2 | 56.618 | −15.752 | 7.867 | 1.00 | 173.09 |
| 12 | CG | PRO A | 2 | 56.407 | −16.439 | 9.184 | 1.00 | 173.72 |
| 13 | C | PRO A | 2 | 55.836 | −16.175 | 5.460 | 1.00 | 167.05 |
| 14 | O | PRO A | 2 | 55.605 | −15.044 | 5.015 | 1.00 | 168.35 |
| 15 | N | GLN A | 3 | 56.252 | −17.165 | 4.696 | 1.00 | 154.29 |
| 16 | CA | GLN A | 3 | 56.695 | −17.039 | 3.302 | 1.00 | 144.07 |
| 17 | CB | GLN A | 3 | 56.716 | −18.462 | 2.775 | 1.00 | 152.72 |
| 18 | CG | GLN A | 3 | 57.593 | −19.352 | 3.656 | 1.00 | 159.56 |
| 19 | CD | GLN A | 3 | 58.812 | −18.642 | 4.253 | 1.00 | 162.34 |
| 20 | OE1 | GLN A | 3 | 59.151 | −17.549 | 3.808 | 1.00 | 167.23 |
| 21 | NE2 | GLN A | 3 | 59.600 | −19.090 | 5.219 | 1.00 | 166.20 |
| 22 | C | GLN A | 3 | 56.117 | −15.992 | 2.286 | 1.00 | 134.57 |
| 23 | O | GLN A | 3 | 56.663 | −15.988 | 1.196 | 1.00 | 145.79 |
| 24 | N | LYS A | 4 | 55.146 | −15.111 | 2.520 | 1.00 | 114.68 |
| 25 | CA | LYS A | 4 | 54.768 | −14.237 | 1.357 | 1.00 | 91.89 |
| 26 | CB | LYS A | 4 | 53.529 | −14.805 | 0.655 | 1.00 | 93.44 |
| 27 | CG | LYS A | 4 | 52.415 | −15.272 | 1.595 | 1.00 | 116.31 |
| 28 | CD | LYS A | 4 | 51.061 | −15.271 | 0.914 | 1.00 | 123.93 |
| 29 | CE | LYS A | 4 | 50.072 | −16.156 | 1.643 | 1.00 | 131.64 |
| 30 | NZ | LYS A | 4 | 49.049 | −16.742 | 0.732 | 1.00 | 135.93 |
| 31 | C | LYS A | 4 | 54.546 | −12.760 | 1.718 | 1.00 | 72.33 |
| 32 | O | LYS A | 4 | 54.002 | −12.486 | 2.790 | 1.00 | 69.94 |
| 33 | N | PRO A | 5 | 54.961 | −11.818 | 0.861 | 1.00 | 46.80 |
| 34 | CD | PRO A | 5 | 55.670 | −12.005 | −0.413 | 1.00 | 34.03 |
| 35 | CA | PRO A | 5 | 54.807 | −10.400 | 1.180 | 1.00 | 35.08 |
| 36 | CB | PRO A | 5 | 55.351 | −9.708 | −0.066 | 1.00 | 27.27 |
| 37 | CG | PRO A | 5 | 56.350 | −10.674 | −0.591 | 1.00 | 18.92 |
| 38 | C | PRO A | 5 | 53.320 | −10.124 | 1.371 | 1.00 | 41.36 |
| 39 | 0 | PRO A | 5 | 52.473 | −10.945 | 1.030 | 1.00 | 59.20 |
| 40 | N | LYS A | 6 | 52.988 | −8.970 | 1.915 | 1.00 | 45.12 |
| 41 | CA | LYS A | 6 | 51.591 | −8.644 | 2.112 | 1.00 | 58.01 |
| 42 | CB | LYS A | 6 | 51.207 | −8.907 | 3.570 | 1.00 | 31.24 |
| 43 | CG | LYS A | 6 | 49.726 | −8.775 | 3.844 | 1.00 | 63.36 |
| 44 | CD | LYS A | 6 | 49.203 | −10.005 | 4.566 | 1.00 | 81.81 |
| 45 | CE | LYS A | 6 | 47.699 | −9.917 | 4.797 | 1.00 | 83.24 |
| 46 | NZ | LYS A | 6 | 47.156 | −11.172 | 5.406 | 1.00 | 80.30 |
| 47 | C | LYS A | 6 | 51.392 | −7.177 | 1.734 | 1.00 | 61.99 |
| 48 | O | LYS A | 6 | 51.822 | −6.280 | 2.460 | 1.00 | 80.08 |
| 49 | N | VAL A | 7 | 50.773 | −6.936 | 0.583 | 1.00 | 46.06 |
| 50 | CA | VAL A | 7 | 50.542 | −5.574 | 0.153 | 1.00 | 39.39 |
| 51 | CB | VAL A | 7 | 49.937 | −5.529 | −1.254 | 1.00 | 45.45 |
| 52 | CG1 | VAL A | 7 | 49.551 | −4.097 | −1.603 | 1.00 | 49.77 |
| 53 | CG2 | VAL A | 7 | 50.947 | −6.057 | −2.270 | 1.00 | 22.27 |
| 54 | C | VAL A | 7 | 49.594 | −4.890 | 1.125 | 1.00 | 44.09 |
| 55 | O | VAL A | 7 | 48.558 | −5.446 | 1.493 | 100 | 37.53 |
| 56 | N | SER A | 8 | 49.970 | −3.686 | 1.546 | 1.00 | 52.74 |
| 57 | CA | SER A | 8 | 49.162 | −2.909 | 2.474 | 1.00 | 53.44 |
| 58 | CB | SER A | 8 | 49.936 | −2.627 | 3.752 | 1.00 | 61.38 |
| 59 | OG | SER A | 8 | 50.799 | −1.517 | 3.558 | 1.00 | 88.92 |
| 60 | C | SER A | 8 | 48.886 | −1.598 | 1.772 | 1.00 | 51.41 |
| 61 | 0 | SER A | 8 | 49.698 | −1.134 | 0.963 | 1.00 | 45.35 |
| 62 | N | LEU A | 9 | 47.753 | −0.988 | 2.080 | 1.00 | 44.36 |
| 63 | CA | LEU A | 9 | 47.422 | 0.260 | 1.422 | 1.00 | 49.82 |
| 64 | CB | LEU A | 9 | 46.027 | 0.193 | 0.778 | 1.00 | 64.30 |
| 65 | CG | LEU A | 9 | 45.511 | −1.067 | 0.080 | 1.00 | 48.97 |
| 66 | CD1 | LEU A | 9 | 44.236 | −0.713 | −0.646 | 1.00 | 42.71 |
| 67 | CD2 | LEU A | 9 | 46.536 | −1.600 | −0.898 | 1.00 | 20.50 |
| 68 | C | LEU A | 9 | 47.429 | 1.408 | 2.405 | 1.00 | 39.97 |
| 69 | O | LEU A | 9 | 47.003 | 1.263 | 3.551 | 1.00 | 31.04 |
| 70 | N | ASN A | 10 | 47.892 | 2.557 | 1.937 | 1.00 | 39.17 |
| 71 | CA | ASN A | 10 | 47.888 | 3.747 | 2.758 | 1.00 | 38.83 |
| 72 | CB | ASN A | 10 | 49.249 | 3.992 | 3.387 | 1.00 | 56.48 |
| 73 | CG | ASN A | 10 | 49.281 | 5.278 | 4.188 | 1.00 | 70.12 |
| 74 | OD1 | ASN A | 10 | 48.500 | 5.445 | 5.135 | 1.00 | 58.68 |
| 75 | ND2 | ASN A | 10 | 50.169 | 6.205 | 3.807 | 1.00 | 68.79 |
| 76 | C | ASN A | 10 | 47.518 | 4.957 | 1.909 | 1.00 | 31.34 |
| 77 | 0 | ASN A | 10 | 48.302 | 5.400 | 1.040 | 1.00 | 26.78 |
| 78 | N | PRO A | 11 | 46.305 | 5.494 | 2.124 | 1.00 | 9.37 |
| 79 | CD | PRO A | 11 | 45.988 | 6.856 | 1.655 | 1.00 | 13.93 |
| 80 | CA | PRO A | 11 | 45.313 | 5.030 | 3.102 | 1.00 | 18.39 |
| 81 | CB | PRO A | 11 | 44.263 | 6.137 | 3.082 | 1.00 | 26.39 |
| 82 | CG | PRO A | 11 | 45.107 | 7.388 | 2.763 | 1.00 | 26.16 |
| 83 | C | PRO A | 11 | 44.718 | 3.662 | 2.745 | 1.00 | 29.16 |
| 84 | O | PRO A | 11 | 44.619 | 3.300 | 1.579 | 1.00 | 42.47 |
| 85 | N | PRO A | 12 | 44.277 | 2.911 | 3.759 | 1.00 | 29.44 |
| 86 | CD | PRO A | 12 | 44.139 | 3.482 | 5.107 | 1.00 | 42.51 |
| 87 | CA | PRO A | 12 | 43.673 | 1.578 | 3.725 | 1.00 | 43.05 |
| 88 | CB | PRO A | 12 | 43.049 | 1.454 | 5.115 | 1.00 | 41.74 |
| 89 | CG | PRO A | 12 | 43.957 | 2.251 | 5.951 | 1.00 | 59.93 |
| 90 | C | PRO A | 12 | 42.625 | 1.358 | 2.645 | 1.00 | 51.72 |
| 91 | 0 | PRO A | 12 | 42.384 | 0.223 | 2.220 | 1.00 | 61.36 |
| 92 | N | TRP A | 13 | 41.985 | 2.444 | 2.231 | 1.00 | 52.05 |
| 93 | CA | TRP A | 13 | 40.926 | 2.405 | 1.232 | 1.00 | 45.67 |
| 94 | CB | TRP A | 13 | 40.423 | 3.818 | 1.033 | 1.00 | 48.38 |
| 95 | CG | TRP A | 13 | 40.354 | 4.497 | 2.343 | 1.00 | 49.00 |
| 96 | CD2 | TRP A | 13 | 39.731 | 3.991 | 3.519 | 1.00 | 33.76 |
| 97 | CE2 | TRP A | 13 | 39.943 | 4.934 | 4.542 | 1.00 | 31.11 |
| 98 | CE3 | TRP A | 13 | 39.013 | 2.827 | 3.809 | 1.00 | 30.50 |
| 99 | CD1 | TRP A | 13 | 40.908 | 5.693 | 2.685 | 1.00 | 46.06 |
| 100 | NE1 | TRP A | 13 | 40.667 | 5.962 | 4.005 | 1.00 | 48.28 |
| 101 | CZ2 | TRP A | 13 | 39.463 | 4.755 | 5.837 | 1.00 | 28.30 |
| 102 | CZ3 | TRP A | 13 | 38.536 | 2.646 | 5.102 | 1.00 | 40.23 |
| 103 | CH2 | TRP A | 13 | 38.764 | 3.610 | 6.100 | 1.00 | 132.97 |
| 104 | C | TRP A | 13 | 41.348 | 1.802 | −0.087 | 1.00 | 47.41 |
| 105 | O | TRP A | 13 | 42.162 | 2.382 | −0.809 | 1.00 | 45.41 |
| 106 | N | ASN A | 14 | 40.796 | 0.627 | −0.386 | 1.00 | 52.08 |
| 107 | CA | ASN A | 14 | 41.102 | −0.084 | −1.622 | 1.00 | 50.75 |
| 108 | CB | ASN A | 14 | 40.891 | −1.578 | −1.434 | 1.00 | 45.92 |
| 109 | CG | ASN A | 14 | 39.442 | −1.920 | −1.257 | 1.00 | 58.48 |
| 110 | OD1 | ASN A | 14 | 38.790 | −1.435 | −0.331 | 1.00 | 59.48 |
| 111 | ND2 | ASN A | 14 | 38.916 | −2.747 | −2.153 | 1.00 | 60.88 |
| 112 | C | ASN A | 14 | 40.171 | 0.433 | −2.716 | 1.00 | 49.24 |
| 113 | O | ASN A | 14 | 40.280 | 0.060 | −3.881 | 1.00 | 55.28 |
| 114 | N | ARG A | 15 | 39.238 | 1.284 | −2.317 | 1.00 | 40.77 |
| 115 | CA | ARG A | 15 | 38.310 | 1.895 | −3.250 | 1.00 | 33.20 |
| 116 | CB | ARG A | 15 | 36.875 | 1.556 | −2.879 | 1.00 | 21.25 |
| 117 | CG | ARG A | 15 | 36.724 | 0.305 | −2.085 | 1.00 | 42.18 |
| 118 | CD | ARG A | 15 | 35.250 | 0.125 | −1.761 | 1.00 | 35.91 |
| 119 | NE | ARG A | 15 | 34.488 | −0.087 | −2.981 | 1.00 | 10.90 |
| 120 | CZ | ARG A | 15 | 33.194 | 0.157 | −3.092 | 1.00 | 35.38 |
| 121 | NH1 | ARG A | 15 | 32.538 | 0.624 | −2.051 | 1.00 | 33.36 |
| 122 | NH2 | ARG A | 15 | 32.563 | −0.078 | −4.231 | 1.00 | 59.41 |
| 123 | C | ARG A | 15 | 38.518 | 3.406 | −3.108 | 1.00 | 32.71 |
| 124 | 0 | ARG A | 15 | 38.262 | 3.995 | −2.058 | 1.00 | 18.86 |
| 125 | N | ILE A | 16 | 38.965 | 4.051 | −4.168 | 1.00 | 25.83 |
| 126 | CA | ILE A | 16 | 39.191 | 5.470 | −4.083 | 1.00 | 22.32 |
| 127 | CB | ILE A | 16 | 40.666 | 5.698 | −4.000 | 1.00 | 4.67 |
| 128 | CG2 | ILE A | 16 | 41.229 | 4.810 | −2.957 | 1.00 | 24.87 |
| 129 | CG1 | ILE A | 16 | 41.319 | 5.326 | −5.326 | 1.00 | 5.49 |
| 130 | CD1 | ILE A | 16 | 42.840 | 5.449 | −5.311 | 1.00 | 5.72 |
| 131 | C | ILE A | 16 | 38.620 | 6.262 | −5.253 | 1.00 | 27.38 |
| 132 | O | ILE A | 16 | 38.407 | 5.729 | −6.332 | 1.00 | 50.75 |
| 133 | N | PHE A | 17 | 38.380 | 7.545 | −5.024 | 1.00 | 29.15 |
| 134 | CA | PHE A | 17 | 37.877 | 8.447 | −6.047 | 1.00 | 14.06 |
| 135 | CB | PHE A | 17 | 37.408 | 9.741 | −5.400 | 1.00 | 10.31 |
| 136 | CG | PHE A | 17 | 36.041 | 9.655 | −4.819 | 1.00 | 5.03 |
| 137 | CD1 | PHE A | 17 | 35.697 | 10.396 | −3.697 | 1.00 | 17.85 |
| 138 | CD2 | PHE A | 17 | 35.071 | 8.885 | −5.431 | 1.00 | 6.30 |
| 139 | CE1 | PHE A | 17 | 34.385 | 10.376 | −3.190 | 1.00 | 28.74 |
| 140 | CE2 | PHE A | 17 | 33.743 | 8.853 | −4.934 | 1.00 | 28.88 |
| 141 | CZ | PHE A | 17 | 33.399 | 9.598 | −3.817 | 1.00 | 19.77 |
| 142 | C | PHE A | 17 | 38.975 | 8.769 | −7.051 | 1.00 | 25.28 |
| 143 | O | PHE A | 17 | 40.159 | 8.770 | −6.717 | 1.00 | 27.78 |
| 144 | N | LYS A | 18 | 38.567 | 9.041 | −8.283 | 1.00 | 38.02 |
| 145 | CA | LYS A | 18 | 39.502 | 9.379 | −9.346 | 1.00 | 41.11 |
| 146 | CB | LYS A | 18 | 38.736 | 9.665 | −10.645 | 1.00 | 37.45 |
| 147 | CG | LYS A | 18 | 39.565 | 10.297 | −11.754 | 1.00 | 38.42 |
| 148 | CD | LYS A | 18 | 38.672 | 10.698 | −12.919 | 1.00 | 71.68 |
| 149 | CE | LYS A | 18 | 39.477 | 11.290 | −14.078 | 1.00 | 82.18 |
| 150 | NZ | LYS A | 18 | 40.148 | 12.572 | −13.720 | 1.00 | 90.91 |
| 151 | C | LYS A | 18 | 40.280 | 10.612 | −8.932 | 1.00 | 42.58 |
| 152 | 0 | LYS A | 18 | 39.707 | 11.549 | −8.376 | 1.00 | 50.79 |
| 153 | N | GLY A | 19 | 41.582 | 10.607 | −9.193 | 1.00 | 38.08 |
| 154 | CA | GLY A | 19 | 42.389 | 11.763 | −8.843 | 1.00 | 50.88 |
| 155 | C | GLY A | 19 | 42.987 | 11.754 | −7.445 | 1.00 | 51.07 |
| 156 | 0 | GLY A | 19 | 43.838 | 12.600 | −7.117 | 1.00 | 53.98 |
| 157 | N | GLU A | 20 | 42.537 | 10.820 | −6.609 | 1.00 | 35.00 |
| 158 | CA | GLU A | 20 | 43.081 | 10.712 | −5.266 | 1.00 | 30.62 |
| 159 | CB | GLU A | 20 | 42.113 | 9.993 | −4.338 | 1.00 | 17.69 |
| 160 | CG | GLU A | 20 | 40.753 | 10.651 | −4.261 | 1.00 | 52.43 |
| 161 | CD | GLU A | 20 | 39.951 | 10.197 | −3.050 | 1.00 | 59.33 |
| 162 | OE1 | GLU A | 20 | 39.832 | 8.970 | −2.842 | 1.00 | 67.80 |
| 163 | OE2 | GLU A | 20 | 39.437 | 11.064 | −2.306 | 1.00 | 52.52 |
| 164 | C | GLU A | 20 | 44.402 | 9.953 | −5.301 | 1.00 | 40.13 |
| 165 | O | GLU A | 20 | 44.789 | 9.367 | −6.321 | 1.00 | 29.35 |
| 166 | N | ASN A | 21 | 45.089 | 9.958 | −4.171 | 1.00 | 39.02 |
| 167 | CA | ASN A | 21 | 46.375 | 9.303 | −4.083 | 1.00 | 35.97 |
| 168 | CB | ASN A | 21 | 47.390 | 10.310 | −3.549 | 1.00 | 52.23 |
| 169 | CG | ASN A | 21 | 47.721 | 11.379 | −4.569 | 1.00 | 60.60 |
| 170 | OD1 | ASN A | 21 | 48.190 | 11.032 | −5.657 | 1.00 | 71.53 |
| 171 | ND2 | ASN A | 21 | 47.493 | 12.658 | −4.253 | 1.00 | 51.64 |
| 172 | C | ASN A | 21 | 46.307 | 8.066 | −3.204 | 1.00 | 39.75 |
| 173 | O | ASN A | 21 | 45.377 | 7.916 | −2.390 | 1.00 | 35.49 |
| 174 | N | VAL A | 22 | 47.263 | 7.160 | −3.393 | 1.00 | 30.65 |
| 175 | CA | VAL A | 22 | 47.311 | 5.934 | −2.597 | 1.00 | 25.06 |
| 176 | CB | VAL A | 22 | 46.241 | 4.918 | −3.040 | 1.00 | 31.80 |
| 177 | CG1 | VAL A | 22 | 46.606 | 4.337 | −4.418 | 1.00 | 36.39 |
| 178 | CG2 | VAL A | 22 | 46.083 | 3.825 | −1.985 | 1.00 | 5.71 |
| 179 | C | VAL A | 22 | 48.678 | 5.312 | −2.761 | 1.00 | 33.39 |
| 180 | O | VAL A | 22 | 49.291 | 5.422 | −3.833 | 1.00 | 34.28 |
| 181 | N | THR A | 23 | 49.168 | 4.669 | −1.704 | 1.00 | 44.00 |
| 182 | CA | THR A | 23 | 50.499 | 4.073 | −1.755 | 1.00 | 47.12 |
| 183 | CB | THR A | 23 | 51.497 | 4.815 | −0.829 | 1.00 | 48.39 |
| 184 | OG1 | THR A | 23 | 51.516 | 6.216 | −1.138 | 1.00 | 51.20 |
| 185 | CG2 | THR A | 23 | 52.903 | 4.243 | −1.015 | 1.00 | 33.54 |
| 186 | C | THR A | 23 | 50.508 | 2.610 | −1.356 | 1.00 | 39.06 |
| 187 | O | THR A | 23 | 50.146 | 2.261 | −0.228 | 1.00 | 40.82 |
| 188 | N | LEU A | 24 | 50.920 | 1.758 | −2.286 | 1.00 | 36.12 |
| 189 | CA | LEU A | 24 | 50.982 | 0.337 | −2.012 | 1.00 | 42.23 |
| 190 | CB | LEU A | 24 | 50.773 | −0.471 | −3.294 | 1.00 | 36.69 |
| 191 | CG | LEU A | 24 | 49.429 | −0.222 | −3.968 | 1.00 | 33.10 |
| 192 | CD1 | LEU A | 24 | 49.240 | −1.186 | −5.117 | 1.00 | 50.66 |
| 193 | CD2 | LEU A | 24 | 48.321 | −0.391 | −2.938 | 1.00 | 31.21 |
| 194 | C | LEU A | 24 | 52.352 | 0.044 | −1.444 | 1.00 | 42.69 |
| 195 | O | LEU A | 24 | 53.364 | 0.492 | −1.991 | 1.00 | 30.60 |
| 196 | N | THR A | 25 | 52.392 | −0.704 | −0.346 | 1.00 | 52.44 |
| 197 | CA | THR A | 25 | 53.667 | −1.046 | 0.263 | 1.00 | 58.07 |
| 198 | CB | THR A | 25 | 53.806 | −0.422 | 1.652 | 1.00 | 62.06 |
| 199 | OG1 | THR A | 25 | 53.423 | 0.958 | 1.601 | 1.00 | 59.04 |
| 200 | CG2 | THR A | 25 | 55.252 | −0.519 | 2.115 | 1.00 | 57.89 |
| 201 | C | THR A | 25 | 53.820 | −2.557 | 0.373 | 1.00 | 58.45 |
| 202 | 0 | THR A | 25 | 52.874 | −3.261 | 0.754 | 1.00 | 57.55 |
| 203 | N | CYS A | 26 | 55.015 | −3.039 | 0.035 | 1.00 | 48.91 |
| 204 | CA | CYS A | 26 | 55.334 | −4.465 | 0.061 | 1.00 | 53.25 |
| 205 | C | CYS A | 26 | 56.187 | −4.793 | 1.272 | 1.00 | 65.21 |
| 206 | O | CYS A | 26 | 57.370 | −4.444 | 1.305 | 1.00 | 62.05 |
| 207 | CB | CYS A | 26 | 56.103 | −4.833 | −1.201 | 1.00 | 58.57 |
| 208 | SG | CYS A | 26 | 56.163 | −6.602 | −1.640 | 1.00 | 76.24 |
| 209 | N | ASN A | 27 | 55.594 | −5.489 | 2.266 | 1.00 | 76.22 |
| 210 | CA | ASN A | 27 | 56.319 | −5.806 | 3.490 | 1.00 | 94.33 |
| 211 | CB | ASN A | 27 | 55.742 | −5.025 | 4.670 | 1.00 | 99.45 |
| 212 | CG | ASN A | 27 | 54.369 | −5.518 | 5.080 | 1.00 | 114.67 |
| 213 | OD1 | ASN A | 27 | 53.835 | −6.457 | 4.490 | 1.00 | 126.31 |
| 214 | ND2 | ASN A | 27 | 53.792 | −4.886 | 6.095 | 1.00 | 113.87 |
| 215 | C | ASN A | 27 | 56.288 | −7.302 | 3.775 | 1.00 | 96.08 |
| 216 | O | ASN A | 27 | 55.477 | −8.061 | 3.274 | 1.00 | 101.25 |
| 217 | N | GLY A | 28 | 57.227 | −7.729 | 4.653 | 1.00 | 92.99 |
| 218 | CA | GLY A | 28 | 57.316 | −9.125 | 5.042 | 1.00 | 93.67 |
| 219 | C | GLY A | 28 | 58.420 | −9.329 | 6.058 | 1.00 | 97.85 |
| 220 | O | GLY A | 28 | 59.153 | −8.393 | 6.368 | 1.00 | 107.57 |
| 221 | N | ASN A | 29 | 58.544 | −10.542 | 6.583 | 1.00 | 96.86 |
| 222 | CA | ASN A | 29 | 59.581 | −10.846 | 7.561 | 1.00 | 94.84 |
| 223 | CB | ASN A | 29 | 59.517 | −12.310 | 7.954 | 1.00 | 99.10 |
| 224 | CG | ASN A | 29 | 58.106 | −12.811 | 8.040 | 1.00 | 116.98 |
| 225 | OD1 | ASN A | 29 | 57.352 | −12.453 | 8.948 | 1.00 | 116.13 |
| 226 | ND2 | ASN A | 29 | 57.726 | −13.631 | 7.077 | 1.00 | 132.36 |
| 227 | C | ASN A | 29 | 60.941 | −10.562 | 6.954 | 1.00 | 97.30 |
| 228 | O | ASN A | 29 | 61.245 | −11.011 | 5.846 | 1.00 | 100.89 |
| 229 | N | ASN A | 30 | 61.761 | −9.821 | 7.683 | 1.00 | 95.34 |
| 230 | CA | ASN A | 30 | 63.090 | −9.479 | 7.209 | 1.00 | 100.29 |
| 231 | CB | ASN A | 30 | 63.751 | −8.483 | 8.165 | 1.00 | 108.20 |
| 232 | CG | ASN A | 30 | 62.971 | −7.188 | 8.288 | 1.00 | 117.57 |
| 233 | OD1 | ASN A | 30 | 62.798 | −6.453 | 7.314 | 1.00 | 118.50 |
| 234 | ND2 | ASN A | 30 | 62.494 | −6.900 | 9.498 | 1.00 | 125.45 |
| 235 | C | ASN A | 30 | 63.979 | −10.709 | 7.086 | 1.00 | 99.61 |
| 236 | O | ASN A | 30 | 65.174 | −10.573 | 6.812 | 1.00 | 111.39 |
| 237 | N | PHE A | 31 | 63.407 | −11.903 | 7.278 | 1.00 | 87.84 |
| 238 | CA | PHE A | 31 | 64.181 | −13.146 | 7.201 | 1.00 | 71.61 |
| 239 | CB | PHE A | 31 | 63.288 | −14.344 | 6.949 | 1.00 | 63.53 |
| 240 | CG | PHE A | 31 | 63.993 | −15.645 | 7.148 | 1.00 | 71.95 |
| 241 | CD1 | PHE A | 31 | 64.017 | −16.257 | 8.395 | 1.00 | 79.52 |
| 242 | CD2 | PHE A | 31 | 64.687 | −16.234 | 6.099 | 1.00 | 82.44 |
| 243 | CE1 | PHE A | 31 | 64.724 | −17.444 | 8.595 | 1.00 | 77.29 |
| 244 | CE2 | PHE A | 31 | 65.400 | −17.420 | 6.284 | 1.00 | 89.06 |
| 245 | CZ | PHE A | 31 | 65.420 | −18.025 | 7.538 | 1.00 | 83.28 |
| 246 | C | PHE A | 31 | 65.252 | −13.102 | 6.111 | 1.00 | 69.17 |
| 247 | 0 | PHE A | 31 | 66.452 | −13.207 | 6.393 | 1.00 | 84.06 |
| 248 | N | PHE A | 32 | 64.809 | −12.971 | 4.865 | 1.00 | 59.43 |
| 249 | CA | PHE A | 32 | 65.736 | −12.851 | 3.750 | 1.00 | 52.65 |
| 250 | CB | PHE A | 32 | 65.255 | −13.673 | 2.565 | 1.00 | 41.66 |
| 251 | CG | PHE A | 32 | 65.585 | −15.134 | 2.659 | 1.00 | 47.56 |
| 252 | CD1 | PHE A | 32 | 64.584 | −16.073 | 2.907 | 1.00 | 44.68 |
| 253 | CD2 | PHE A | 32 | 66.897 | −15.575 | 2.492 | 1.00 | 51.53 |
| 254 | CE1 | PHE A | 32 | 64.883 | −17.433 | 2.989 | 1.00 | 43.51 |
| 255 | CE2 | PHE A | 32 | 67.208 | −16.938 | 2.580 | 1.00 | 51.46 |
| 256 | CZ | PHE A | 32 | 66.196 | −17.866 | 2.824 | 1.00 | 52.70 |
| 257 | C | PHE A | 32 | 65.794 | −11.375 | 3.345 | 1.00 | 58.83 |
| 258 | 0 | PHE A | 32 | 64.773 | −10.698 | 3.278 | 1.00 | 56.92 |
| 259 | N | GLU A | 33 | 66.990 | −10.875 | 3.080 | 1.00 | 62.55 |
| 260 | CA | GLU A | 33 | 67.152 | −9.482 | 2.695 | 1.00 | 69.28 |
| 261 | CB | GLU A | 33 | 68.640 | −9.164 | 2.586 | 1.00 | 88.52 |
| 262 | CG | GLU A | 33 | 69.291 | −8.758 | 3.901 | 1.00 | 69.46 |
| 263 | CD | GLU A | 33 | 70.782 | −8.585 | 3.753 | 1.00 | 84.74 |
| 264 | OE1 | GLU A | 33 | 71.225 | −8.087 | 2.697 | 1.00 | 88.47 |
| 265 | OE2 | GLU A | 33 | 71.516 | −8.945 | 4.693 | 1.00 | 105.94 |
| 266 | C | GLU A | 33 | 66.436 | −9.106 | 1.391 | 1.00 | 64.49 |
| 267 | O | GLU A | 33 | 66.268 | −9.936 | 0.490 | 1.00 | 58.36 |
| 268 | N | VAL A | 34 | 66.045 | −7.837 | 1.292 | 1.00 | 53.30 |
| 269 | CA | VAL A | 34 | 65.345 | −7.329 | 0.123 | 1.00 | 45.11 |
| 270 | CB | VAL A | 34 | 63.852 | −7.116 | 0.440 | 1.00 | 48.71 |
| 271 | CG1 | VAL A | 34 | 63.143 | −6.517 | −0.760 | 1.00 | 64.13 |
| 272 | CG2 | VAL A | 34 | 63.207 | −8.418 | 0.846 | 1.00 | 12.46 |
| 273 | C | VAL A | 34 | 65.902 | −5.992 | −0.379 | 1.00 | 53.63 |
| 274 | O | VAL A | 34 | 65.671 | −4.944 | 0.233 | 1.00 | 61.37 |
| 275 | N | SER A | 35 | 66.614 | −6.020 | −1.499 | 1.00 | 49.22 |
| 276 | CA | SER A | 35 | 67.155 | −4.790 | −2.063 | 1.00 | 59.53 |
| 277 | CB | SER A | 35 | 68.650 | −4.931 | −2.345 | 1.00 | 70.72 |
| 278 | OG | SER A | 35 | 68.886 | −5.671 | −3.532 | 1.00 | 78.30 |
| 279 | C | SER A | 35 | 66.438 | −4.442 | −3.362 | 1.00 | 65.71 |
| 280 | O | SER A | 35 | 66.894 | −3.576 | −4.106 | 1.00 | 79.59 |
| 281 | N | SER A | 36 | 65.325 | −5.122 | −3.631 | 1.00 | 71.64 |
| 282 | CA | SER A | 36 | 64.546 | −4.889 | −4.849 | 1.00 | 68.57 |
| 283 | CB | SER A | 36 | 65.307 | −5.378 | −6.084 | 1.00 | 72.44 |
| 284 | OG | SER A | 36 | 65.260 | −6.790 | −6.186 | 1.00 | 79.50 |
| 285 | C | SER A | 36 | 63.197 | −5.593 | −4.805 | 1.00 | 63.12 |
| 286 | 0 | SER A | 36 | 63.070 | −6.691 | −4.265 | 1.00 | 64.24 |
| 287 | N | THR A | 37 | 62.189 | −4.961 | −5.391 | 1.00 | 60.88 |
| 288 | CA | THR A | 37 | 60.850 | −5.529 | −5.417 | 1.00 | 47.23 |
| 289 | CB | THR A | 37 | 59.864 | −4.621 | −4.687 | 1.00 | 45.28 |
| 290 | OG1 | THR A | 37 | 60.421 | −4.251 | −3.420 | 1.00 | 61.45 |
| 291 | CG2 | THR A | 37 | 58.521 | −5.336 | −4.477 | 1.00 | 29.55 |
| 292 | C | THR A | 37 | 60.363 | −5.712 | −6.854 | 1.00 | 48.75 |
| 293 | O | THR A | 37 | 60.992 | −5.240 | −7.809 | 1.00 | 46.94 |
| 294 | N | LYS A | 38 | 59.244 | −6.406 | −7.002 | 1.00 | 44.37 |
| 295 | CA | LYS A | 38 | 58.658 | −6.655 | −8.308 | 1.00 | 27.76 |
| 296 | CB | LYS A | 38 | 58.820 | −8.117 | −8.693 | 1.00 | 41.96 |
| 297 | CG | LYS A | 38 | 59.620 | −8.368 | −9.929 | 1.00 | 34.76 |
| 298 | CD | LYS A | 38 | 61.033 | −7.877 | −9.768 | 1.00 | 57.35 |
| 299 | CE | LYS A | 38 | 61.944 | −8.534 | −10.793 | 1.00 | 65.86 |
| 300 | NZ | LYS A | 38 | 61.835 | −10.029 | −10.698 | 1.00 | 34.19 |
| 301 | C | LYS A | 38 | 57.184 | −6.351 | −8.160 | 1.00 | 39.95 |
| 302 | O | LYS A | 38 | 56.489 | −6.975 | −7.341 | 1.00 | 37.00 |
| 303 | N | TRP A | 39 | 56.705 | −5.384 | −8.934 | 1.00 | 51.66 |
| 304 | CA | TRP A | 39 | 55.292 | −5.012 | −8.878 | 1.00 | 46.32 |
| 305 | CB | TRP A | 39 | 55.130 | −3.498 | −8.736 | 1.00 | 37.04 |
| 306 | CG | TRP A | 39 | 55.477 | −3.023 | −7.372 | 1.00 | 41.46 |
| 307 | CD2 | TRP A | 39 | 54.615 | −3.022 | −6.233 | 1.00 | 14.22 |
| 308 | CE2 | TRP A | 39 | 55.360 | −2.534 | −5.141 | 1.00 | 5.00 |
| 309 | CE3 | TRP A | 39 | 53.284 | −3.389 | −6.025 | 1.00 | 27.18 |
| 310 | CD1 | TRP A | 39 | 56.685 | −2.548 | −6.940 | 1.00 | 38.68 |
| 311 | NE1 | TRP A | 39 | 56.622 | −2.252 | −5.597 | 1.00 | 29.64 |
| 312 | CZ2 | TRP A | 39 | 54.819 | −2.396 | −3.864 | 1.00 | 25.70 |
| 313 | CZ3 | TRP A | 39 | 52.745 | −3.253 | −4.742 | 1.00 | 38.07 |
| 314 | CH2 | TRP A | 39 | 53.514 | −2.761 | −3.683 | 1.00 | 26.72 |
| 315 | C | TRP A | 39 | 54.531 | −5.501 | −10.102 | 1.00 | 35.61 |
| 316 | O | TRP A | 39 | 55.080 | −5.587 | −11.209 | 1.00 | 19.21 |
| 317 | N | PHE A | 40 | 53.266 | −5.833 | −9.898 | 1.00 | 11.64 |
| 318 | CA | PHE A | 40 | 52.480 | −6.327 | −10.994 | 1.00 | 6.70 |
| 319 | CB | PHE A | 40 | 52.340 | −7.853 | −10.899 | 1.00 | 19.61 |
| 320 | CG | PHE A | 40 | 53.644 | −8.595 | −11.029 | 1.00 | 22.54 |
| 321 | CD1 | PHE A | 40 | 54.445 | −8.807 | −9.935 | 1.00 | 38.72 |
| 322 | CD2 | PHE A | 40 | 54.100 | −9.019 | −12.262 | 1.00 | 33.22 |
| 323 | CE1 | PHE A | 40 | 55.673 | −9.409 | −10.067 | 1.00 | 29.68 |
| 324 | CE2 | PHE A | 40 | 55.334 | −9.625 | −12.395 | 1.00 | 36.71 |
| 325 | CZ | PHE A | 40 | 56.117 | −9.818 | −11.296 | 1.00 | 35.88 |
| 326 | C | PHE A | 40 | 51.108 | −5.687 | −11.031 | 1.00 | 37.09 |
| 327 | 0 | PHE A | 40 | 50.263 | −5.931 | −10.145 | 1.00 | 42.94 |
| 328 | N | HIS A | 41 | 50.902 | −4.846 | −12.046 | 1.00 | 41.78 |
| 329 | CA | HIS A | 41 | 49.604 | −4.207 | −12.245 | 1.00 | 41.13 |
| 330 | CB | HIS A | 41 | 49.734 | −2.831 | −12.878 | 1.00 | 45.29 |
| 331 | CG | HIS A | 41 | 48.457 | −2.055 | −12.857 | 1.00 | 22.20 |
| 332 | CD2 | HIS A | 41 | 48.178 | −0.784 | −13.225 | 1.00 | 33.03 |
| 333 | ND1 | HIS A | 41 | 47.288 | −2.576 | −12.347 | 1.00 | 36.74 |
| 334 | CE1 | HIS A | 41 | 46.341 | −1.656 | −12.396 | 1.00 | 31.11 |
| 335 | NE2 | HIS A | 41 | 46.856 | −0.558 | −12.924 | 1.00 | 55.47 |
| 336 | C | HIS A | 41 | 48.903 | −5.131 | −13.215 | 1.00 | 29.92 |
| 337 | 0 | HIS A | 41 | 49.468 | −5.495 | −14.246 | 1.00 | 19.98 |
| 338 | N | ASN A | 42 | 47.688 | −5.532 | −12.876 | 1.00 | 26.06 |
| 339 | CA | ASN A | 42 | 46.968 | −6.458 | −13.730 | 1.00 | 33.33 |
| 340 | CB | ASN A | 42 | 46.265 | −5.680 | −14.834 | 1.00 | 17.02 |
| 341 | CG | ASN A | 42 | 45.045 | −4.956 | −14.334 | 1.00 | 32.02 |
| 342 | OD1 | ASN A | 42 | 44.490 | −5.335 | −13.274 | 1.00 | 29.56 |
| 343 | ND2 | ASN A | 42 | 44.618 | −3.945 | −15.101 | 1.00 | 13.43 |
| 344 | C | ASN A | 42 | 47.924 | −7.520 | −14.333 | 1.00 | 38.30 |
| 345 | O | ASN A | 42 | 47.954 | −7.738 | −15.548 | 1.00 | 35.70 |
| 346 | N | GLY A | 43 | 48.723 | −8.150 | −13.476 | 1.00 | 39.92 |
| 347 | CA | GLY A | 43 | 49.640 | −9.182 | −13.926 | 1.00 | 32.70 |
| 348 | C | GLY A | 43 | 50.880 | −8.741 | −14.686 | 1.00 | 36.33 |
| 349 | O | GLY A | 43 | 51.786 | −9.544 | −14.891 | 1.00 | 40.53 |
| 350 | N | SER A | 44 | 50.946 | −7.489 | −15.117 | 1.00 | 34.42 |
| 351 | CA | SER A | 44 | 52.124 | −7.048 | −15.862 | 1.00 | 44.42 |
| 352 | CB | SER A | 44 | 51.738 | −6.055 | −16.957 | 1.00 | 57.35 |
| 353 | OG | SER A | 44 | 50.928 | −6.677 | −17.942 | 1.00 | 77.66 |
| 354 | C | SER A | 44 | 53.128 | −6.402 | −14.947 | 1.00 | 42.83 |
| 355 | O | SER A | 44 | 52.755 | −5.629 | −14.072 | 1.00 | 48.20 |
| 356 | N | LEU A | 45 | 54.404 | −6.713 | −15.153 | 1.00 | 46.97 |
| 357 | CA | LEU A | 45 | 55.472 | −6.160 | −14.322 | 1.00 | 46.50 |
| 358 | CB | LEU A | 45 | 56.819 | −6.805 | −14.665 | 1.00 | 33.26 |
| 359 | CG | LEU A | 45 | 58.045 | −6.173 | −13.999 | 1.00 | 34.18 |
| 360 | CD1 | LEU A | 45 | 57.912 | −6.233 | −12.476 | 1.00 | 58.24 |
| 361 | CD2 | LEU A | 45 | 59.287 | −6.896 | −14.445 | 1.00 | 39.00 |
| 362 | C | LEU A | 45 | 55.581 | −4.658 | −14.501 | 1.00 | 46.94 |
| 363 | O | LEU A | 45 | 55.627 | −4.180 | −15.611 | 1.00 | 34.79 |
| 364 | N | SER A | 46 | 55.612 | −3.922 | −13.400 | 1.00 | 60.24 |
| 365 | CA | SER A | 46 | 55.721 | −2.478 | −13.456 | 1.00 | 54.99 |
| 366 | CB | SER A | 46 | 55.040 | −1.857 | −12.240 | 1.00 | 58.08 |
| 367 | OG | SER A | 46 | 55.175 | −0.446 | −12.254 | 1.00 | 81.72 |
| 368 | C | SER A | 46 | 57.191 | −2.132 | −13.442 | 1.00 | 58.15 |
| 369 | O | SER A | 46 | 58.023 | −2.965 | −13.100 | 1.00 | 70.14 |
| 370 | N | GLU A | 47 | 57.513 | −0.902 | −13.819 | 1.00 | 57.45 |
| 371 | CA | GLU A | 47 | 58.900 | −0.462 | −13.822 | 1.00 | 65.18 |
| 372 | CB | GLU A | 47 | 59.070 | 0.748 | −14.735 | 1.00 | 75.20 |
| 373 | CG | GLU A | 47 | 58.981 | 0.417 | −16.207 | 1.00 | 98.71 |
| 374 | CD | GLU A | 47 | 59.119 | 1.648 | −17.075 | 1.00 | 118.62 |
| 375 | OE1 | GLU A | 47 | 58.207 | 2.505 | −17.038 | 1.00 | 131.08 |
| 376 | OE2 | GLU A | 47 | 60.140 | 1.761 | −17.788 | 1.00 | 126.76 |
| 377 | C | GLU A | 47 | 59.352 | −0.109 | −12.408 | 1.00 | 63.59 |
| 378 | O | GLU A | 47 | 60.551 | −0.117 | −12.108 | 1.00 | 67.24 |
| 379 | N | GLU A | 48 | 58.382 | 0.193 | −11.546 | 1.00 | 53.32 |
| 380 | CA | GLU A | 48 | 58.653 | 0.539 | −10.156 | 1.00 | 55.75 |
| 381 | CB | GLU A | 48 | 57.343 | 0.802 | −9.422 | 1.00 | 58.11 |
| 382 | CG | GLU A | 48 | 57.530 | 1.166 | −7.964 | 1.00 | 86.91 |
| 383 | CD | GLU A | 48 | 58.235 | 2.501 | −7.781 | 1.00 | 104.21 |
| 384 | OE1 | GLU A | 48 | 57.545 | 3.548 | −7.752 | 1.00 | 116.45 |
| 385 | OE2 | GLU A | 48 | 59.483 | 2.499 | −7.681 | 1.00 | 107.36 |
| 386 | C | GLU A | 48 | 59.403 | −0.581 | −9.442 | 1.00 | 57.10 |
| 387 | O | GLU A | 48 | 59.016 | −1.744 | −9.518 | 1.00 | 64.00 |
| 388 | N | THR A | 49 | 60.471 | −0.230 | −8.735 | 1.00 | 54.72 |
| 389 | CA | THR A | 49 | 61.253 | −1.238 | −8.024 | 1.00 | 63.14 |
| 390 | CB | THR A | 49 | 62.646 | −1.380 | −8.647 | 1.00 | 63.41 |
| 391 | OG1 | THR A | 49 | 62.980 | −0.174 | −9.345 | 1.00 | 62.51 |
| 392 | CG2 | THR A | 49 | 62.677 | −2.552 | −9.603 | 1.00 | 64.13 |
| 393 | C | THR A | 49 | 61.415 | −1.015 | −6.521 | 1.00 | 62.86 |
| 394 | 0 | THR A | 49 | 62.066 | −1.806 | −5.839 | 1.00 | 69.10 |
| 395 | N | ASN A | 50 | 60.821 | 0.052 | −6.003 | 1.00 | 54.53 |
| 396 | CA | ASN A | 50 | 60.918 | 0.341 | −4.580 | 1.00 | 61.75 |
| 397 | CB | ASN A | 50 | 60.864 | 1.860 | −4.331 | 1.00 | 76.64 |
| 398 | CG | ASN A | 50 | 61.974 | 2.623 | −5.065 | 1.00 | 80.85 |
| 399 | OD1 | ASN A | 50 | 63.136 | 2.211 | −5.063 | 1.00 | 73.80 |
| 400 | ND2 | ASN A | 50 | 61.615 | 3.745 | −5.686 | 1.00 | 83.17 |
| 401 | C | ASN A | 50 | 59.795 | −0.365 | −3.827 | 1.00 | 60.45 |
| 402 | O | ASN A | 50 | 58.718 | −0.579 | −4.361 | 1.00 | 64.31 |
| 403 | N | SER A | 51 | 60.053 | −0.723 | −2.579 | 1.00 | 64.42 |
| 404 | CA | SER A | 51 | 59.067 | −1.417 | −1.767 | 1.00 | 69.19 |
| 405 | CB | SER A | 51 | 59.649 | −1.690 | −0.383 | 1.00 | 83.18 |
| 406 | OG | SER A | 51 | 60.136 | −0.498 | 0.207 | 1.00 | 98.67 |
| 407 | C | SER A | 51 | 57.757 | −0.651 | −1.633 | 1.00 | 72.43 |
| 408 | O | SER A | 51 | 56.779 | −1.160 | −1.076 | 1.00 | 75.57 |
| 409 | N | SER A | 52 | 57.738 | 0.577 | −2.138 | 1.00 | 72.75 |
| 410 | CA | SER A | 52 | 56.535 | 1.391 | −2.069 | 1.00 | 72.90 |
| 411 | CB | SER A | 52 | 56.766 | 2.600 | −1.165 | 1.00 | 77.27 |
| 412 | OG | SER A | 52 | 57.143 | 2.182 | 0.137 | 1.00 | 104.21 |
| 413 | C | SER A | 52 | 56.106 | 1.858 | −3.449 | 1.00 | 69.39 |
| 414 | 0 | SER A | 52 | 56.806 | 2.617 | −4.114 | 1.00 | 66.69 |
| 415 | N | LEU A | 53 | 54.950 | 1.376 | −3.883 | 1.00 | 70.92 |
| 416 | CA | LEU A | 53 | 54.405 | 1.754 | −5.176 | 1.00 | 66.04 |
| 417 | CB | LEU A | 53 | 53.747 | 0.542 | −5.841 | 1.00 | 64.62 |
| 418 | CG | LEU A | 53 | 52.954 | 0.809 | −7.120 | 1.00 | 57.82 |
| 419 | CD1 | LEU A | 53 | 53.761 | 1.661 | −8.068 | 1.00 | 53.42 |
| 420 | CD2 | LEU A | 53 | 52.593 | −0.504 | −7.758 | 1.00 | 52.64 |
| 421 | C | LEU A | 53 | 53.383 | 2.879 | −4.975 | 1.00 | 58.35 |
| 422 | O | LEU A | 53 | 52.319 | 2.688 | −4.348 | 1.00 | 38.80 |
| 423 | N | ASN A | 54 | 53.708 | 4.055 | −5.500 | 1.00 | 47.61 |
| 424 | CA | ASN A | 54 | 52.820 | 5.195 | −5.344 | 1.00 | 60.77 |
| 425 | CB | ASN A | 54 | 53.638 | 6.453 | −5.089 | 1.00 | 62.70 |
| 426 | CG | ASN A | 54 | 54.433 | 6.376 | −3.804 | 1.00 | 72.45 |
| 427 | OD1 | ASN A | 54 | 53.865 | 6.317 | −2.708 | 1.00 | 70.70 |
| 428 | ND2 | ASN A | 54 | 55.761 | 6.373 | −3.927 | 1.00 | 70.84 |
| 429 | C | ASN A | 54 | 51.905 | 5.420 | −6.534 | 1.00 | 55.42 |
| 430 | 0 | ASN A | 54 | 52.308 | 5.246 | −7.678 | 1.00 | 52.22 |
| 431 | N | ILE A | 55 | 50.669 | 5.807 | −6.241 | 1.00 | 47.52 |
| 432 | CA | ILE A | 55 | 49.681 | 6.089 | −7.267 | 1.00 | 48.77 |
| 433 | CB | ILE A | 55 | 48.459 | 5.177 | −7.138 | 1.00 | 48.20 |
| 434 | CG2 | ILE A | 55 | 47.338 | 5.670 | −8.055 | 1.00 | 64.85 |
| 435 | CG1 | ILE A | 55 | 48.848 | 3.746 | −7.493 | 1.00 | 17.58 |
| 436 | CD1 | ILE A | 55 | 47.667 | 2.811 | −7.511 | 1.00 | 22.96 |
| 437 | C | ILE A | 55 | 49.242 | 7.528 | −7.070 | 1.00 | 53.16 |
| 438 | O | ILE A | 55 | 48.602 | 7.862 | −6.062 | 1.00 | 50.16 |
| 439 | N | VAL A | 56 | 49.576 | 8.374 | −8.041 | 1.00 | 53.12 |
| 440 | CA | VAL A | 56 | 49.238 | 9.784 | −7.939 | 1.00 | 55.98 |
| 441 | CB | VAL A | 56 | 50.475 | 10.660 | −8.216 | 1.00 | 53.06 |
| 442 | CG1 | VAL A | 56 | 50.160 | 12.096 | −7.893 | 1.00 | 67.53 |
| 443 | CG2 | VAL A | 56 | 51.656 | 10.181 | −7.396 | 1.00 | 38.93 |
| 444 | C | VAL A | 56 | 48.109 | 10.214 | −8.867 | 1.00 | 54.49 |
| 445 | O | VAL A | 56 | 48.152 | 9.979 | −10.075 | 1.00 | 42.98 |
| 446 | N | ASN A | 57 | 47.094 | 10.850 | −8.287 | 1.00 | 63.99 |
| 447 | CA | ASN A | 57 | 45.941 | 11.323 | −9.041 | 1.00 | 72.29 |
| 448 | CB | ASN A | 57 | 46.339 | 12.495 | −9.945 | 1.00 | 88.41 |
| 449 | CG | ASN A | 57 | 46.916 | 13.667 | −9.157 | 1.00 | 95.72 |
| 450 | OD1 | ASN A | 57 | 46.274 | 14.182 | −8.236 | 1.00 | 90.65 |
| 451 | ND2 | ASN A | 57 | 48.133 | 14.089 | −9.512 | 1.00 | 90.43 |
| 452 | C | ASN A | 57 | 45.413 | 10.169 | −9.866 | 1.00 | 67.43 |
| 453 | O | ASN A | 57 | 45.349 | 10.232 | −11.089 | 1.00 | 76.76 |
| 454 | N | ALA A | 58 | 45.046 | 9.107 | −9.161 | 1.00 | 65.44 |
| 455 | CA | ALA A | 58 | 44.526 | 7.893 | −9.764 | 1.00 | 59.50 |
| 456 | CB | ALA A | 58 | 43.813 | 7.065 | −8.715 | 1.00 | 51.61 |
| 457 | C | ALA A | 58 | 43.591 | ' 8.128 | −10.925 | 1.00 | 56.95 |
| 458 | O | ALA A | 58 | 42.696 | 8.976 | −10.860 | 1.00 | 59.56 |
| 459 | N | LYS A | 59 | 43.815 | 7.356 | −11.984 | 1.00 | 54.03 |
| 460 | CA | LYS A | 59 | 42.999 | 7.392 | −13.188 | 1.00 | 56.80 |
| 461 | CB | LYS A | 59 | 43.897 | 7.371 | −14.432 | 1.00 | 52.13 |
| 462 | CG | LYS A | 59 | 44.932 | 8.485 | −14.454 | 1.00 | 75.19 |
| 463 | CD | LYS A | 59 | 46.010 | 8.238 | −15.500 | 1.00 | 87.07 |
| 464 | CE | LYS A | 59 | 47.122 | 9.272 | −15.379 | 1.00 | 101.21 |
| 465 | N2 | LYS A | 59 | 48.271 | 8.958 | −16.271 | 1.00 | 106.25 |
| 466 | C | LYS A | 59 | 42.171 | 6.106 | −13.095 | 1.00 | 51.59 |
| 467 | 0 | LYS A | 59 | 42.354 | 5.318 | −12.167 | 1.00 | 37.92 |
| 468 | N | PHE A | 60 | 41.241 | 5.888 | −14.015 | 1.00 | 54.25 |
| 469 | CA | PHE A | 60 | 40.470 | 4.657 | −13.946 | 1.00 | 48.76 |
| 470 | CB | PHE A | 60 | 39.250 | 4.729 | −14.854 | 1.00 | 54.70 |
| 471 | CG | PHE A | 60 | 38.304 | 5.832 | −14.506 | 1.00 | 37.20 |
| 472 | CD1 | PHE A | 60 | 38.495 | 7.117 | −15.002 | 1.00 | 29.43 |
| 473 | CD2 | PHE A | 60 | 37.215 | 5.583 | −13.684 | 1.00 | 37.72 |
| 474 | CE1 | PHE A | 60 | 37.606 | 8.134 | −14.687 | 1.00 | 40.04 |
| 475 | CE2 | PHE A | 60 | 36.315 | 6.597 | −13.358 | 1.00 | 23.38 |
| 476 | CZ | PHE A | 60 | 36.510 | 7.875 | −13.860 | 1.00 | 30.49 |
| 477 | C | PHE A | 60 | 41.388 | 3.529 | −14.397 | 1.00 | 45.95 |
| 478 | O | PHE A | 60 | 41.263 | 2.389 | −13.957 | 1.00 | 37.39 |
| 479 | N | GLU A | 61 | 42.326 | 3.865 | −15.276 | 1.00 | 48.08 |
| 480 | CA | GLU A | 61 | 43.279 | 2.887 | −15.782 | 1.00 | 61.34 |
| 481 | CB | GLU A | 61 | 44.195 | 3.525 | −16.842 | 1.00 | 72.31 |
| 482 | CG | GLU A | 61 | 43.508 | 3.900 | −18.170 | 1.00 | 91.97 |
| 483 | CD | GLU A | 61 | 42.606 | 5.127 | −18.061 | 1.00 | 102.49 |
| 484 | OE1 | GLU A | 61 | 43.123 | 6.227 | −17.758 | 1.00 | 100.34 |
| 485 | OE2 | GLU A | 61 | 41.381 | 4.993 | −18.283 | 1.00 | 100.98 |
| 486 | C | GLU A | 61 | 44.116 | 2.335 | −14.625 | 1.00 | 57.54 |
| 487 | O | GLU A | 61 | 44.781 | 1.306 | −14.754 | 1.00 | 53.72 |
| 488 | N | ASP A | 62 | 44.078 | 3.027 | −13.493 | 1.00 | 48.67 |
| 489 | CA | ASP A | 62 | 44.837 | 2.597 | −12.330 | 1.00 | 40.78 |
| 490 | CB | ASP A | 62 | 45.141 | 3.784 | −11.407 | 1.00 | 58.38 |
| 491 | CG | ASP A | 62 | 46.284 | 4.654 | −11.920 | 1.00 | 55.63 |
| 492 | OD1 | ASP A | 62 | 47.343 | 4.090 | −12.281 | 1.00 | 50.67 |
| 493 | OD2 | ASP A | 62 | 46.122 | 5.898 | −11.947 | 1.00 | 65.51 |
| 494 | C | ASP A | 62 | 44.104 | 1.519 | −11.547 | 1.00 | 32.95 |
| 495 | O | ASP A | 62 | 44.699 | 0.839 | −10.711 | 1.00 | 33.25 |
| 496 | N | SER A | 63 | 42.808 | 1.374 | −11.800 | 1.00 | 27.08 |
| 497 | CA | SER A | 63 | 42.038 | 0.346 | −11.111 | 1.00 | 24.76 |
| 498 | CB | SER A | 63 | 40.574 | 0.381 | −11.542 | 1.00 | 32.81 |
| 499 | OG | SER A | 63 | 39.976 | 1.632 | −11.254 | 1.00 | 57.02 |
| 500 | C | SER A | 63 | 42.639 | −1.002 | −11.500 | 1.00 | 36.75 |
| 501 | 0 | SER A | 63 | 43.468 | −1.094 | −12.419 | 1.00 | 31.64 |
| 502 | N | GLY A | 64 | 42.236 | −2.057 | −10.809 | 1.00 | 34.81 |
| 503 | CA | GLY A | 64 | 42.790 | −3.348 | −11.160 | 1.00 | 42.02 |
| 504 | C | GLY A | 64 | 43.349 | −4.182 | −10.021 | 1.00 | 50.47 |
| 505 | O | GLY A | 64 | 43.011 | −4.005 | −8.850 | 1.00 | 48.47 |
| 506 | N | GLU A | 65 | 44.224 | −5.107 | −10.387 | 1.00 | 51.85 |
| 507 | CA | GLU A | 65 | 44.836 | −6.021 | −9.436 | 1.00 | 43.33 |
| 508 | CB | GLU A | 65 | 44.691 | −7.448 | −9.953 | 1.00 | 52.77 |
| 509 | CG | GLU A | 65 | 45.405 | −8.502 | −9.135 | 1.00 | 52.38 |
| 510 | CD | GLU A | 65 | 45.636 | −9.792 | −9.907 | 1.00 | 58.39 |
| 511 | OE1 | GLU A | 65 | 46.528 | −9.798 | −10.801 | 1.00 | 43.40 |
| 512 | OE2 | GLU A | 65 | 44.917 | −10.782 | −9.610 | 1.00 | 51.74 |
| 513 | C | GLU A | 65 | 46.309 | −5.711 | −9.240 | 1.00 | 45.24 |
| 514 | O | GLU A | 65 | 47.057 | −5.550 | −10.212 | 1.00 | 39.10 |
| 515 | N | TYR A | 66 | 46.726 | −5.644 | −7.981 | 1.00 | 40.60 |
| 516 | CA | TYR A | 66 | 48.119 | −5.363 | −7.664 | 1.00 | 35.48 |
| 517 | CB | TYR A | 66 | 48.259 | −4.029 | −6.936 | 1.00 | 18.93 |
| 518 | CG | TYR A | 66 | 48.056 | −2.817 | −7.810 | 1.00 | 24.42 |
| 519 | CD1 | TYR A | 66 | 46.801 | −2.218 | −7.927 | 1.00 | 27.62 |
| 520 | CE1 | TYR A | 66 | 46.618 | −1.068 | −8.707 | 1.00 | 20.61 |
| 521 | CD2 | TYR A | 66 | 49.131 | −2.240 | −8.493 | 1.00 | 20.62 |
| 522 | CE2 | TYR A | 66 | 48.968 | −1.093 | −9.268 | 1.00 | 19.14 |
| 523 | CZ | TYR A | 66 | 47.708 | −0.506 | −9.375 | 1.00 | 37.26 |
| 524 | OH | TYR A | 66 | 47.546 | 0.631 | −10.149 | 1.00 | 20.86 |
| 525 | C | TYR A | 66 | 48.692 | −6.454 | −6.789 | 1.00 | 35.81 |
| 526 | 0 | TYR A | 66 | 47.992 | −6.973 | −5.888 | 1.00 | 26.54 |
| 527 | N | LYS A | 67 | 49.950 | −6.803 | −7.071 | 1.00 | 20.27 |
| 528 | CA | LYS A | 67 | 50.672 | −7.827 | −6.316 | 1.00 | 42.77 |
| 529 | CB | LYS A | 67 | 50.277 | −9.255 | −6.735 | 1.00 | 42.46 |
| 530 | CG | LYS A | 67 | 50.131 | −9.502 | −8.223 | 1.00 | 70.28 |
| 531 | CD | LYS A | 67 | 49.867 | −10.987 | −8.489 | 1.00 | 66.50 |
| 532 | CE | LYS A | 67 | 49.313 | −11.233 | −9.888 | 1.00 | 73.20 |
| 533 | NZ | LYS A | 67 | 49.387 | −12.668 | −10.255 | 1.00 | 55.01 |
| 534 | C | LYS A | 67 | 52.161 | −7.648 | −6.480 | 1.00 | 49.35 |
| 535 | O | LYS A | 67 | 52.656 | −7.502 | −7.596 | 1.00 | 49.27 |
| 536 | N | CYS A | 68 | 52.872 | −7.632 | −5.359 | 1.00 | 55.09 |
| 537 | CA | CYS A | 68 | 54.314 | −7.450 | −5.384 | 1.00 | 54.74 |
| 538 | C | CYS A | 68 | 54.966 | −8.793 | −5.171 | 1.00 | 42.46 |
| 539 | O | CYS A | 68 | 54.285 | −9.748 | −4.779 | 1.00 | 18.20 |
| 540 | CB | CYS A | 68 | 54.750 | −6.451 | −4.309 | 1.00 | 64.47 |
| 541 | SG | CYS A | 68 | 54.393 | −6.915 | −2.586 | 1.00 | 100.38 |
| 542 | N | GLN A | 69 | 56.267 | −8.876 | −5.444 | 1.00 | 26.58 |
| 543 | CA | GLN A | 69 | 56.968 | −10.142 | −5.299 | 1.00 | 26.57 |
| 544 | CB | GLN A | 69 | 56.693 | −10.973 | −6.556 | 1.00 | 15.18 |
| 545 | CG | GLN A | 69 | 57.877 | −11.704 | −7.156 | 1.00 | 38.54 |
| 546 | CD | GLN A | 69 | 57.573 | −12.192 | −8.567 | 1.00 | 36.61 |
| 547 | OE1 | GLN A | 69 | 56.511 | −12.787 | −8.816 | 1.00 | 18.42 |
| 548 | NE2 | GLN A | 69 | 58.497 | −11.941 | −9.498 | 1.00 | 36.58 |
| 549 | C | GLN A | 69 | 58.472 | −10.000 | −5.034 | 1.00 | 41.83 |
| 550 | O | GLN A | 69 | 59.174 | −9.343 | −5.804 | 1.00 | 24.51 |
| 551 | N | HIS A | 70 | 58.951 | −10.602 | −3.938 | 1.00 | 59.31 |
| 552 | CA | HIS A | 70 | 60.375 | −10.552 | −3.568 | 1.00 | 54.15 |
| 553 | CB | HIS A | 70 | 60.550 | −10.461 | −2.050 | 1.00 | 49.67 |
| 554 | CG | HIS A | 70 | 60.025 | −9.194 | −1.451 | 1.00 | 57.18 |
| 555 | CD2 | HIS A | 70 | 59.202 | −8.984 | −0.397 | 1.00 | 57.85 |
| 556 | ND1 | HIS A | 70 | 60.376 | −7.946 | −1.918 | 1.00 | 60.44 |
| 557 | CE1 | HIS A | 70 | 59.796 | −7.021 | −1.178 | 1.00 | 62.11 |
| 558 | NE2 | HIS A | 70 | 59.078 | −7.624 | −0.246 | 1.00 | 69.17 |
| 559 | C | HIS A | 70 | 61.065 | −11.814 | −4.055 | 1.00 | 45.74 |
| 560 | O | HIS A | 70 | 60.390 | −12.760 | −4.466 | 1.00 | 48.46 |
| 561 | N | GLN A | 71 | 62.397 | −11.824 | −4.020 | 1.00 | 34.41 |
| 562 | CA | GLN A | 71 | 63.170 | −12.998 | −4.434 | 1.00 | 34.74 |
| 563 | CB | GLN A | 71 | 64.631 | −12.609 | −4.631 | 1.00 | 28.64 |
| 564 | CG | GLN A | 71 | 65.521 | −13.746 | −5.061 | 1.00 | 28.58 |
| 565 | CD | GLN A | 71 | 67.021 | −13.427 | −4.933 | 1.00 | 44.36 |
| 566 | OE1 | GLN A | 71 | 67.508 | −12.432 | −5.447 | 1.00 | 24.64 |
| 567 | NE2 | GLN A | 71 | 67.749 | −14.289 | −4.246 | 1.00 | 82.76 |
| 568 | C | GLN A | 71 | 63.098 | •14.123 | −3.376 | 1.00 | 42.41 |
| 569 | 0 | GLN A | 71 | 63.036 | −13.876 | −2.168 | 1.00 | 58.78 |
| 570 | N | GLN A | 72 | 63.116 | −15.369 | −3.822 | 1.00 | 36.65 |
| 571 | CA | GLN A | 72 | 63.062 | −16.493 | −2.873 | 1.00 | 56.26 |
| 572 | CB | GLN A | 72 | 64.225 | −16.440 | −1.894 | 1.00 | 66.55 |
| 573 | CG | GLN A | 72 | 65.522 | −16.962 | −2.419 | 1.00 | 74.96 |
| 574 | CD | GLN A | 72 | 66.614 | −16.771 | −1.409 | 1.00 | 81.18 |
| 575 | OE1 | GLN A | 72 | 66.932 | −15.625 | −1.027 | 1.00 | 46.66 |
| 576 | NE2 | GLN A | 72 | 67.196 | −17.886 | −0.944 | 1.00 | 86.79 |
| 577 | C | GLN A | 72 | 61.791 | −16.614 | −2.044 | 1.00 | 47.57 |
| 578 | 0 | GLN A | 72 | 61.763 | −17.306 | −1.035 | 1.00 | 53.55 |
| 579 | N | VAL A | 73 | 60.740 | −15.937 | −2.457 | 1.00 | 25.14 |
| 580 | CA | VAL A | 73 | 59.499 | −16.027 | −1.737 | 1.00 | 27.76 |
| 581 | CB | VAL A | 73 | 59.434 | −14.865 | −0.770 | 1.00 | 24.20 |
| 582 | CG1 | VAL A | 73 | 58.034 | −14.638 | −0.284 | 1.00 | 61.77 |
| 583 | CG2 | VAL A | 73 | 60.349 | −15.143 | 0.383 | 1.00 | 48.95 |
| 584 | C | VAL A | 73 | 58.330 | −16.018 | −2.730 | 1.00 | 38.97 |
| 585 | O | VAL A | 73 | 58.405 | −15.385 | −3.793 | 1.00 | 50.48 |
| 586 | N | ASN A | 74 | 57.261 | −16.735 | −2.392 | 1.00 | 30.12 |
| 587 | CA | ASN A | 74 | 56.080 | −16.794 | −3.251 | 1.00 | 54.75 |
| 588 | CB | ASN A | 74 | 55.023 | −17.677 | −2.576 | 1.00 | 65.77 |
| 589 | CG | ASN A | 74 | 55.444 | −19.138 | −2.523 | 1.00 | 77.30 |
| 590 | OD1 | ASN A | 74 | 55.408 | −19.840 | −3.538 | 1.00 | 66.94 |
| 591 | ND2 | ASN A | 74 | 55.871 | −19.586 | −1.341 | 1.00 | 98.68 |
| 592 | C | ASN A | 74 | 55.514 | −15.392 | −3.593 | 1.00 | 61.15 |
| 593 | O | ASN A | 74 | 55.945 | −14.384 | −3.027 | 1.00 | 65.55 |
| 594 | N | GLU A | 75 | 54.553 | −15.322 | −4.523 | 1.00 | 62.19 |
| 595 | CA | GLU A | 75 | 53.971 | −14.057 | −4.951 | 1.00 | 63.45 |
| 596 | CB | GLU A | 75 | 53.296 | −14.213 | −6.314 | 1.00 | 82.08 |
| 597 | CG | GLU A | 75 | 54.237 | −14.649 | −7.425 | 1.00 | 101.46 |
| 598 | CD | GLU A | 75 | 53.533 | −14.804 | −8.759 | 1.00 | 105.08 |
| 599 | OE1 | GLU A | 75 | 52.308 | −14.564 | −8.815 | 1.00 | 85.23 |
| 600 | OE2 | GLU A | 75 | 54.204 | −15.167 | −9.747 | 1.00 | 113.01 |
| 601 | C | GLU A | 75 | 52.979 | −13.531 | −3.920 | 1.00 | 46.81 |
| 602 | O | GLU A | 75 | 52.180 | −14.279 | −3.376 | 1.00 | 63.28 |
| 603 | N | SER A | 76 | 53.008 | −12.216 | −3.667 | 1.00 | 44.23 |
| 604 | CA | SER A | 76 | 52.104 | −11.602 | −2.698 | 1.00 | 44.01 |
| 605 | CB | SER A | 76 | 52.265 | −10.080 | −2.698 | 1.00 | 52.11 |
| 606 | OG | SER A | 76 | 51.674 | −9.502 | −3.854 | 1.00 | 49.14 |
| 607 | C | SER A | 76 | 50.658 | −11.942 | −3.036 | 1.00 | 40.30 |
| 608 | O | SER A | 76 | 50.351 | −12.409 | −4.122 | 1.00 | 37.45 |
| 609 | N | GLU A | 77 | 49.787 | −11.733 | −2.074 | 1.00 | 47.16 |
| 610 | CA | GLU A | 77 | 48.373 | −11.965 | −2.345 | 1.00 | 50.65 |
| 611 | CB | GLU A | 77 | 47.596 | −12.125 | −1.037 | 1.00 | 64.23 |
| 612 | CG | GLU A | 77 | 48.051 | −13.299 | −0.184 | 1.00 | 100.08 |
| 613 | CD | GLU A | 77 | 49.143 | −12.917 | 0.795 | 1.00 | 121.85 |
| 614 | OE1 | GLU A | 77 | 49.535 | −11.731 | 0.817 | 1.00 | 113.34 |
| 615 | OE2 | GLU A | 77 | 49.607 | −13.804 | 1.543 | 1.00 | 135.86 |
| 616 | C | GLU A | 77 | 47.780 | −10.837 | −3.181 | 1.00 | 57.00 |
| 617 | O | GLU A | 77 | 48.203 | −9.696 | −2.996 | 1.00 | 70.10 |
| 618 | N | PRO A | 78 | 46.905 | −11.147 | −4.089 | 1.00 | 60.36 |
| 619 | CD | PRO A | 78 | 46.349 | −12.453 | −4.468 | 1.00 | 76.75 |
| 620 | CA | PRO A | 78 | 46.348 | −10.084 | −4.915 | 1.00 | 45.94 |
| 621 | CB | PRO A | 78 | 45.380 | −10.826 | −5.830 | 1.00 | 63.85 |
| 622 | CG | PRO A | 78 | 45.954 | −12.210 | −5.903 | 1.00 | 74.36 |
| 623 | C | PRO A | 78 | 45.640 | −9.054 | −4.055 | 1.00 | 46.99 |
| 624 | O | PRO A | 78 | 45.047 | −9.380 | −3.014 | 1.00 | 44.78 |
| 625 | N | VAL A | 79 | 45.723 | −7.806 | −4.493 | 1.00 | 38.75 |
| 626 | CA | VAL A | 79 | 45.066 | −6.708 | −3.808 | 1.00 | 46.98 |
| 627 | CB | VAL A | 79 | 46.101 | −5.836 | −3.067 | 1.00 | 48.31 |
| 628 | CG1 | VAL A | 79 | 45.700 | −4.371 | −3.099 | 1.00 | 58.66 |
| 629 | CG2 | VAL A | 79 | 46.195 | −6.289 | −1.639 | 1.00 | 43.93 |
| 630 | C | VAL A | 79 | 44.344 | −5.925 | −4.907 | 1.00 | 41.63 |
| 631 | O | VAL A | 79 | 44.974 | −5.465 | −5.869 | 1.00 | 31.43 |
| 632 | N | TYR A | 80 | 43.024 | −5.799 | −4.786 | 1.00 | 36.26 |
| 633 | CA | TYR A | 80 | 42.258 | −5.105 | −5.807 | 1.00 | 43.25 |
| 634 | CB | TYR A | 80 | 40.986 | −5.889 | −6.142 | 1.00 | 43.58 |
| 635 | CG | TYR A | 80 | 41.315 | −7.289 | −6.619 | 1.00 | 62.18 |
| 636 | CD1 | TYR A | 80 | 41.297 | −8.370 | −5.734 | 1.00 | 59.18 |
| 637 | CE1 | TYR A | 80 | 41.695 | −9.639 | −6.138 | 1.00 | 63.70 |
| 638 | CD2 | TYR A | 80 | 41.738 | −7.521 | −7.932 | 1.00 | 64.60 |
| 639 | CE2 | TYR A | 80 | 42.141 | −8.793 | −8.341 | 1.00 | 72.36 |
| 640 | CZ | TYR A | 80 | 42.116 | −9.843 | −7.439 | 1.00 | 71.99 |
| 641 | OH | TYR A | 80 | 42.511 | −11.096 | −7.848 | 1.00 | 86.14 |
| 642 | C | TYR A | 80 | 41.920 | −3.677 | −5.468 | 1.00 | 39.91 |
| 643 | O | TYR A | 80 | 41.299 | −3.392 | −4.445 | 1.00 | 39.73 |
| 644 | N | LEU A | 81 | 42.350 | −2.791 | −6.356 | 1.00 | 42.49 |
| 645 | CA | LEU A | 81 | 42.130 | −1.367 | −6.236 | 1.00 | 39.83 |
| 646 | CB | LEU A | 81 | 43.462 | −0.645 | −6.434 | 1.00 | 29.82 |
| 647 | CG | LEU A | 81 | 43.433 | 0.870 | −6.520 | 1.00 | 35.37 |
| 648 | CD1 | LEU A | 81 | 42.862 | 1.449 | −5.246 | 1.00 | 54.76 |
| 649 | CD2 | LEU A | 81 | 44.841 | 1.363 | −6.750 | 1.00 | 51.99 |
| 650 | C | LEU A | 81 | 41.113 | −0.953 | −7.307 | 1.00 | 40.44 |
| 651 | O | LEU A | 81 | 41.218 | −1.335 | −8.479 | 1.00 | 44.23 |
| 652 | N | GLU A | 82 | 40.119 | −0.175 | −6.903 | 1.00 | 34.19 |
| 653 | CA | GLU A | 82 | 39.108 | 0.266 | −7.850 | 1.00 | 43.16 |
| 654 | CB | GLU A | 82 | 37.833 | −0.540 | −7.615 | 1.00 | 43.29 |
| 655 | CG | GLU A | 82 | 36.683 | −0.166 | −8.517 | 1.00 | 64.74 |
| 656 | CD | GLU A | 82 | 35.546 | −1.157 | −8.415 | 1.00 | 79.52 |
| 657 | OE1 | GLU A | 82 | 35.177 | −1.505 | −7.272 | 1.00 | 89.05 |
| 658 | OE2 | GLU A | 82 | 35.023 | −1.586 | −9.468 | 1.00 | 95.85 |
| 659 | C | GLU A | 82 | 38.802 | 1.778 | −7.803 | 1.00 | 36.44 |
| 660 | O | GLU A | 82 | 38.213 | 2.268 | −6.840 | 1.00 | 20.88 |
| 661 | N | VAL A | 83 | 39.200 | 2.505 | −8.849 | 1.00 | 17.32 |
| 662 | CA | VAL A | 83 | 38.957 | 3.947 | −8.947 | 1.00 | 20.25 |
| 663 | CB | VAL A | 83 | 39.842 | 4.587 | −10.012 | 1.00 | 16.19 |
| 664 | CG1 | VAL A | 83 | 39.647 | 6.071 | −9.995 | 1.00 | 22.13 |
| 665 | CG2 | VAL A | 83 | 41.280 | 4.219 | −9.786 | 1.00 | 30.28 |
| 666 | C | VAL A | 83 | 37.503 | 4.274 | −9.323 | 1.00 | 24.88 |
| 667 | O | VAL A | 83 | 36.946 | 3.744 | −10.285 | 1.00 | 47.06 |
| 668 | N | PHE A | 84 | 36.894 | 5.162 | −8.555 | 1.00 | 14.13 |
| 669 | CA | PHE A | 84 | 35.520 | 5.575 | −8.800 | 1.00 | 26.86 |
| 670 | CB | PHE A | 84 | 34.646 | 5.374 | −7.564 | 1.00 | 13.44 |
| 671 | CG | PHE A | 84 | 34.475 | 3.964 | −7.163 | 1.00 | 34.33 |
| 672 | CD1 | PHE A | 84 | 35.571 | 3.183 | −6.840 | 1.00 | 45.55 |
| 673 | CD2 | PHE A | 84 | 33.205 | 3.420 | −7.070 | 1.00 | 44.61 |
| 674 | CE1 | PHE A | 84 | 35.405 | 1.865 | −6.433 | 1.00 | 65.24 |
| 675 | CE2 | PHE A | 84 | 33.022 | 2.106 | −6.667 | 1.00 | 42.90 |
| 676 | CZ | PHE A | 84 | 34.122 | 1.325 | −6.343 | 1.00 | 65.66 |
| 677 | C | PHE A | 84 | 35.462 | 7.061 | −9.146 | 1.00 | 45.25 |
| 678 | 0 | PHE A | 84 | 36.490 | 7.760 | −9.178 | 1.00 | 38.34 |
| 679 | N | SER A | 85 | 34.234 | 7.512 | −9.399 | 1.00 | 46.62 |
| 680 | CA | SER A | 85 | 33.919 | 8.899 | −9.712 | 1.00 | 37.65 |
| 681 | CB | SER A | 85 | 34.232 | 9.236 | −11.159 | 1.00 | 40.40 |
| 682 | OG | SER A | 85 | 34.067 | 10.629 | −11.358 | 1.00 | 35.89 |
| 683 | C | SER A | 85 | 32.438 | 9.035 | −9.467 | 1.00 | 22.78 |
| 684 | O | SER A | 85 | 31.630 | 8.613 | −10.264 | 1.00 | 37.65 |
| 685 | N | ASP A | 86 | 32.098 | 9.604 | −8.326 | 1.00 | 14.50 |
| 686 | CA | ASP A | 86 | 30.713 | 9.771 | −7.919 | 1.00 | 9.43 |
| 687 | CB | ASP A | 86 | 30.133 | 8.405 | −7.554 | 1.00 | 12.45 |
| 688 | CG | ASP A | 86 | 28.611 | 8.418 | −7.427 | 1.00 | 51.60 |
| 689 | OD1 | ASP A | 86 | 28.054 | 9.224 | −6.645 | 1.00 | 55.89 |
| 690 | OD2 | ASP A | 86 | 27.960 | 7.606 | −8.115 | 1.00 | 79.60 |
| 691 | C | ASP A | 86 | 30.746 | 10.707 | −6.680 | 1.00 | 28.99 |
| 692 | O | ASP A | 86 | 31.827 | 11.076 | −6.199 | 1.00 | 31.21 |
| 693 | N | TRP A | 87 | 29.583 | 11.104 | −6.171 | 1.00 | 14.11 |
| 694 | CA | TRP A | 87 | 29.543 | 11.991 | −5.024 | 1.00 | 41.60 |
| 695 | CB | TRP A | 87 | 28.161 | 12.643 | −4.893 | 1.00 | 67.93 |
| 696 | CG | TRP A | 87 | 27.790 | 13.509 | −6.057 | 1.00 | 79.67 |
| 697 | CD2 | TRP A | 87 | 28.221 | 14.858 | −6.312 | 1.00 | 97.15 |
| 698 | CE2 | TRP A | 87 | 27.641 | 15.256 | −7.532 | 1.00 | 98.87 |
| 699 | CE3 | TRP A | 87 | 29.040 | 15.762 | −5.626 | 1.00 | 106.42 |
| 700 | CD1 | TRP A | 87 | 26.995 | 13.162 | −7.102 | 1.00 | 73.69 |
| 701 | NE1 | TRP A | 87 | 26.898 | 14.205 | −7.994 | 1.00 | 66.94 |
| 702 | CZ2 | TRP A | 87 | 27.851 | 16.513 | −8.083 | 1.00 | 111.64 |
| 703 | CZ3 | TRP A | 87 | 29.246 | 17.010 | −6.174 | 1.00 | 116.11 |
| 704 | CH2 | TRP A | 87 | 28.654 | 17.375 | −7.392 | 1.00 | 119.60 |
| 705 | C | TRP A | 87 | 29.900 | 11.273 | −3.735 | 1.00 | 44.35 |
| 706 | O | TRP A | 87 | 30.629 | 11.811 | −2.915 | 1.00 | 41.60 |
| 707 | N | LEU A | 88 | 29.368 | 10.071 | −3.546 | 1.00 | 50.46 |
| 708 | CA | LEU A | 88 | 29.659 | 9.311 | −2.340 | 1.00 | 36.94 |
| 709 | CB | LEU A | 88 | 28.394 | 9.038 | −1.530 | 1.00 | 4.59 |
| 710 | CG | LEU A | 88 | 27.705 | 10.219 | −0.861 | 1.00 | 21.69 |
| 711 | CD1 | LEU A | 88 | 26.690 | 9.649 | 0.161 | 1.00 | 11.07 |
| 712 | CD2 | LEU A | 88 | 28.720 | 11.117 | −0.155 | 1.00 | 21.48 |
| 713 | C | LEU A | 88 | 30.289 | 7.981 | −2.655 | 1.00 | 28.38 |
| 714 | O | LEU A | 88 | 29.805 | 7.248 | −3.508 | 1.00 | 29.21 |
| 715 | N | LEU A | 89 | 31.370 | 7.676 | −1.948 | 1.00 | 45.92 |
| 716 | CA | LEU A | 89 | 32.077 | 6.409 | −2.092 | 1.00 | 35.41 |
| 717 | CB | LEU A | 89 | 33.474 | 6.637 | −2.623 | 1.00 | 12.22 |
| 718 | CG | LEU A | 89 | 34.333 | 5.406 | −2.839 | 1.00 | 32.05 |
| 719 | CD1 | LEU A | 89 | 33.477 | 4.296 | −3.405 | 1.00 | 25.06 |
| 720 | CD2 | LEU A | 89 | 35.530 | 5.774 | −3.801 | 1.00 | 25.39 |
| 721 | C | LEU A | 89 | 32.153 | 5.786 | −0.721 | 1.00 | 21.36 |
| 722 | 0 | LEU A | 89 | 32.513 | 6.454 | 0.234 | 1.00 | 48.70 |
| 723 | N | LEU A | 90 | 31.757 | 4.528 | −0.602 | 1.00 | 31.16 |
| 724 | CA | LEU A | 90 | 31.798 | 3.848 | 0.690 | 1.00 | 20.27 |
| 725 | CB | LEU A | 90 | 30.677 | 2.856 | 0.796 | 1.00 | 4.59 |
| 726 | CG | LEU A | 90 | 30.650 | 2.184 | 2.145 | 1.00 | 5.03 |
| 727 | CD1 | LEU A | 90 | 30.229 | 3.221 | 3.170 | 1.00 | 5.42 |
| 728 | CD2 | LEU A | 90 | 29.601 | 1.067 | 2.147 | 1.00 | 18.53 |
| 729 | C | LEU A | 90 | 33.099 | 3.096 | 0.757 | 1.00 | 29.48 |
| 730 | O | LEU A | 90 | 33.291 | 2.129 | 0.017 | 1.00 | 49.50 |
| 731 | N | GLN A | 91 | 34.004 | 3.557 | 1.612 | 1.00 | 33.11 |
| 732 | CA | GLN A | 91 | 35.308 | 2.930 | 1.724 | 1.00 | 34.25 |
| 733 | CB | GLN A | 91 | 36.389 | 4.007 | 1.872 | 1.00 | 29.25 |
| 734 | CG | GLN A | 91 | 36.487 | 4.900 | 0.664 | 1.00 | 4.72 |
| 735 | CD | GLN A | 91 | 37.564 | 5.950 | 0.795 | 1.00 | 24.07 |
| 736 | OE1 | GLN A | 91 | 37.492 | 6.821 | 1.657 | 1.00 | 33.85 |
| 737 | NE2 | GLN A | 91 | 38.566 | 5.885 | −0.075 | 1.00 | 25.90 |
| 738 | C | GLN A | 91 | 35.339 | 1.961 | 2.892 | 1.00 | 39.46 |
| 739 | O | GLN A | 91 | 34.607 | 2.134 | 3.875 | 1.00 | 41.80 |
| 740 | N | ALA A | 92 | 36.162 | 0.922 | 2.774 | 1.00 | 33.12 |
| 741 | CA | ALA A | 92 | 36.256 | −0.064 | 3.844 | 1.00 | 44.90 |
| 742 | CB | ALA A | 92 | 35.425 | −1 .288 | 3.498 | 1.00 | 44.46 |
| 743 | C | ALA A | 92 | 37.696 | −0.476 | 4.151 | 1.00 | 45.76 |
| 744 | O | ALA A | 92 | 38.544 | −0.586 | 3.253 | 1.00 | 51.50 |
| 745 | N | SER A | 93 | 37.962 | −0.702 | 5.432 | 1.00 | 48.03 |
| 746 | CA | SER A | 93 | 39.286 | −1.103 | 5.881 | 1.00 | 52.40 |
| 747 | CB | SER A | 93 | 39.265 | −1.381 | 7.380 | 1.00 | 48.05 |
| 748 | OG | SER A | 93 | 38.305 | −2.377 | 7.689 | 1.00 | 47.42 |
| 749 | C | SER A | 93 | 39.671 | −2.364 | 5.142 | 1.00 | 49.91 |
| 750 | O | SER A | 93 | 40.837 | −2.593 | 4.836 | 1.00 | 54.70 |
| 751 | N | ALA A | 94 | 38.659 | −3.170 | 4.856 | 1.00 | 47.64 |
| 752 | CA | ALA A | 94 | 38.830 | −4.430 | 4.161 | 1.00 | 40.55 |
| 753 | CB | ALA A | 94 | 39.567 | −5.404 | 5.048 | 1.00 | 44.76 |
| 754 | C | ALA A | 94 | 37.441 | −4.968 | 3.823 | 1.00 | 53.40 |
| 755 | 0 | ALA A | 94 | 36.554 | −5.033 | 4.688 | 1.00 | 40.13 |
| 756 | N | GLU A | 95 | 37.249 | −5.347 | 2.565 | 1.00 | 63.75 |
| 757 | CA | GLU A | 95 | 35.964 | −5.865 | 2.125 | 1.00 | 61.37 |
| 758 | CB | GLU A | 95 | 35.952 | −5.940 | 0.603 | 1.00 | 77.66 |
| 759 | CG | GLU A | 95 | 36.118 | −4.576 | −0.059 | 1.00 | 76.66 |
| 760 | CD | GLU A | 95 | 36.098 | −4.640 | −1.582 | 1.00 | 96.68 |
| 761 | OE1 | GLU A | 95 | 36.111 | −3.565 | −2.217 | 1.00 | 107.34 |
| 762 | OE2 | GLU A | 95 | 36.073 | −5.754 | −2.151 | 1.00 | 100.79 |
| 763 | C | GLU A | 95 | 35.680 | −7.231 | 2.750 | 1.00 | 54.28 |
| 764 | O | GLU A | 95 | 34.527 | −7.667 | 2.838 | 1.00 | 42.98 |
| 765 | N | VAL A | 96 | 36.739 | −7.903 | 3.193 | 1.00 | 61.87 |
| 766 | CA | VAL A | 96 | 36.604 | −9.205 | 3.851 | 1.00 | 61.01 |
| 767 | CB | VAL A | 96 | 36.933 | −10.354 | 2.897 | 1.00 | 56.77 |
| 768 | CG1 | VAL A | 96 | 36.541 | −11.670 | 3.536 | 1.00 | 39.60 |
| 769 | CG2 | VAL A | 96 | 36.215 | −10.149 | 1.576 | 1.00 | 53.03 |
| 770 | C | VAL A | 96 | 37.520 | −9.306 | 5.084 | 1.00 | 56.97 |
| 771 | O | VAL A | 96 | 38.751 | −9.202 | 4.985 | 1.00 | 45.37 |
| 772 | N | VAL A | 97 | 36.900 | −9.512 | 6.241 | 1.00 | 53.01 |
| 773 | CA | VAL A | 97 | 37.614 | −9.606 | 7.499 | 1.00 | 56.01 |
| 774 | CB | VAL A | 97 | 37.188 | −8.448 | 8.435 | 1.00 | 57.61 |
| 775 | CG1 | VAL A | 97 | 37.790 | −8.628 | 9.802 | 1.00 | 87.18 |
| 776 | CG2 | VAL A | 97 | 37.637 | −7.122 | 7.858 | 1.00 | 68.07 |
| 777 | C | VAL A | 97 | 37.372 | −10.944 | 8.209 | 1.00 | 61.52 |
| 778 | O | VAL A | 97 | 36.278 | −11.542 | 8.158 | 1.00 | 38.67 |
| 779 | N | MET A | 98 | 38.412 | −11.411 | 8.879 | 1.00 | 64.69 |
| 780 | CA | MET A | 98 | 38.335 | −12.644 | 9.633 | 1.00 | 62.72 |
| 781 | CB | MET A | 98 | 39.745 | −13.081 | 10.013 | 1.00 | 71.28 |
| 782 | CG | MET A | 98 | 39.901 | −14.561 | 10.252 | 1.00 | 94.38 |
| 783 | SD | MET A | 98 | 39.346 | −15.500 | 8.803 | 1.00 | 75.29 |
| 784 | CE | MET A | 98 | 37.821 | −16.041 | 9.448 | 1.00 | 93.84 |
| 785 | C | MET A | 98 | 37.520 | −12.336 | 10.894 | 1.00 | 58.92 |
| 786 | O | MET A | 98 | 37.748 | −11.318 | 11.552 | 1.00 | 61.33 |
| 787 | N | GLU A | 99 | 36.573 | −13.201 | 11.241 | 1.00 | 61.37 |
| 788 | CA | GLU A | 99 | 35.762 | −12.971 | 12.436 | 1.00 | 59.89 |
| 789 | CB | GLU A | 99 | 34.950 | −14.211 | 12.797 | 1.00 | 52.02 |
| 790 | CG | GLU A | 99 | 34.153 | −14.039 | 14.072 | 1.00 | 64.75 |
| 791 | CD | GLU A | 99 | 33.366 | −15.285 | 14.456 | 1.00 | 90.18 |
| 792 | OE1 | GLU A | 99 | 33.983 | −16.375 | 14.536 | 1.00 | 106.04 |
| 793 | OE2 | GLU A | 99 | 32.137 | −15.170 | 14.685 | 1.00 | 84.66 |
| 794 | C | GLU A | 99 | 36.621 | −12.587 | 13.633 | 1.00 | 66.15 |
| 795 | O | GLU A | 99 | 37.583 | −13.284 | 13.972 | 1.00 | 81.55 |
| 796 | N | GLY A | 100 | 36.265 | −11.478 | 14.271 | 1.00 | 58.33 |
| 797 | CA | GLY A | 100 | 37.016 | −11.021 | 15.419 | 1.00 | 54.87 |
| 798 | C | GLY A | 100 | 37.943 | −9.872 | 15.088 | 1.00 | 54.97 |
| 799 | O | GLY A | 100 | 38.293 | −9.087 | 15.960 | 1.00 | 69.91 |
| 800 | N | GLN A | 101 | 38.367 | −9.772 | 13.836 | 1.00 | 54.17 |
| 801 | CA | GLN A | 101 | 39.237 | −8.671 | 13.445 | 1.00 | 57.48 |
| 802 | CB | GLN A | 101 | 39.942 | −8.998 | 12.125 | 1.00 | 60.93 |
| 803 | CG | GLN A | 101 | 41.016 | −10.063 | 12.237 | 1.00 | 76.51 |
| 804 | CD | GLN A | 101 | 42.059 | −9.719 | 13.290 | 1.00 | 90.62 |
| 805 | OE1 | GLN A | 101 | 41.825 | −9.891 | 14.490 | 1.00 | 100.86 |
| 806 | NE2 | GLN A | 101 | 43.212 | −9.215 | 12.846 | 1.00 | 85.92 |
| 807 | C | GLN A | 101 | 38.445 | −7.353 | 13.311 | 1.00 | 56.15 |
| 808 | O | GLN A | 101 | 37.210 | −7.337 | 13.344 | 1.00 | 44.69 |
| 809 | N | PRO A | 102 | 39.151 | −6.222 | 13.180 | 1.00 | 56.36 |
| 810 | CD | PRO A | 102 | 40.600 | −5.991 | 13.326 | 1.00 | 60.77 |
| 811 | CA | PRO A | 102 | 38.438 | −4.954 | 13.048 | 1.00 | 55.67 |
| 812 | CB | PRO A | 102 | 39:483 | −3.933 | 13.458 | 1.00 | 47.18 |
| 813 | CG | PRO A | 102 | 40.742 | −4.538 | 12.918 | 1.00 | 47.65 |
| 814 | C | PRO A | 102 | 37.915 | −4.716 | 11.631 | 1.00 | 58.48 |
| 815 | O | PRO A | 102 | 38.567 | −5.036 | 10.635 | 1.00 | 58.71 |
| 816 | N | LEU A | 103 | 36.719 | −4.151 | 11.569 | 1.00 | 61.34 |
| 817 | CA | LEU A | 103 | 36.041 | −3.833 | 10.320 | 1.00 | 47.87 |
| 818 | CB | LEU A | 103 | 34.728 | −4.610 | 10.248 | 1.00 | 54.63 |
| 819 | CG | LEU A | 103 | 33.811 | −4.304 | 9.073 | 1.00 | 54.99 |
| 820 | CD1 | LEU A | 103 | 34.546 | −4.596 | 7.767 | 1.00 | 57.13 |
| 821 | CD2 | LEU A | 103 | 32.537 | −5.130 | 9.206 | 1.00 | 49.78 |
| 822 | C | LEU A | 103 | 35.761 | −2.332 | 10.359 | 1.00 | 39.79 |
| 823 | 0 | LEU A | 103 | 35.046 | −1.847 | 11.254 | 1.00 | 21.76 |
| 824 | N | PHE A | 104 | 36.300 | −1.598 | 9.391 | 1.00 | 18.87 |
| 825 | CA | PHE A | 104 | 36.111 | −0.157 | 9.393 | 1.00 | 30.54 |
| 826 | CB | PHE A | 104 | 37.466 | 0.500 | 9.616 | 1.00 | 41.57 |
| 827 | CG | PHE A | 104 | 37.385 | 1.948 | 9.969 | 1.00 | 54.55 |
| 828 | CD1 | PHE A | 104 | 37.282 | 2.351 | 11.301 | 1.00 | 72.72 |
| 829 | CD2 | PHE A | 104 | 37.441 | 2.917 | 8.975 | 1.00 | 57.49 |
| 830 | CE1 | PHE A | 104 | 37.227 | 3.706 | 11.640 | 1.00 | 70.88 |
| 831 | CE2 | PHE A | 104 | 37.385 | 4.273 | 9.297 | 1.00 | 60.17 |
| 832 | CZ | PHE A | 104 | 37.285 | 4.670 | 10.633 | 1.00 | 66.74 |
| 833 | C | PHE A | 104 | 35.466 | 0.437 | 8.137 | 1.00 | 35.62 |
| 834 | 0 | PHE A | 104 | 36.079 | 0.468 | 7.066 | 1.00 | 37.03 |
| 835 | N | LEU A | 105 | 34.234 | 0.922 | 8.269 | 1.00 | 36.14 |
| 836 | CA | LEU A | 105 | 33.541 | 1.550 | 7.144 | 1.00 | 48.60 |
| 837 | CB | LEU A | 105 | 32.073 | 1.154 | 7.125 | 1.00 | 46.94 |
| 838 | CG | LEU A | 105 | 31.870 | −0.297 | 6.725 | 1.00 | 52.90 |
| 839 | CD1 | LEU A | 105 | 30.385 | −0.564 | 6.516 | 1.00 | 46.80 |
| 840 | CD2 | LEU A | 105 | 32.663 | −0.562 | 5.442 | 1.00 | 53.92 |
| 841 | C | LEU A | 105 | 33.658 | 3.072 | 7.195 | 1.00 | 46.53 |
| 842 | O | LEU A | 105 | 33.938 | 3.645 | 8.241 | 1.00 | 50.68 |
| 843 | N | ARG A | 106 | 33.406 | 3.725 | 6.066 | 1.00 | 45.66 |
| 844 | CA | ARG A | 106 | 33.539 | 5.172 | 5.962 | 1.00 | 26.49 |
| 845 | CB | ARG A | 106 | 35.021 | 5.513 | 5.786 | 1.00 | 36.08 |
| 846 | CG | ARG A | 106 | 35.354 | 6.951 | 5.450 | 1.00 | 14.61 |
| 847 | CD | ARG A | 106 | 36.798 | 7.050 | 4.915 | 1.00 | 5.55 |
| 848 | NE | ARG A | 106 | 37.271 | 8.435 | 4.831 | 1.00 | 20.40 |
| 849 | CZ | ARG A | 106 | 38.344 | 8.828 | 4.148 | 1.00 | 31.42 |
| 850 | NH1 | ARG A | 106 | 39.064 | 7.946 | 3.479 | 1.00 | 46.98 |
| 851 | NH2 | ARG A | 106 | 38.700 | 10.107 | 4.129 | 1.00 | 48.19 |
| 852 | C | ARG A | 106 | 32.770 | 5.674 | 4.756 | 1.00 | 33.55 |
| 853 | O | ARG A | 106 | 32.812 | 5.073 | 3.681 | 1.00 | 52.61 |
| 854 | N | CYS A | 107 | 32.077 | 6.789 | 4.929 | 1.00 | 27.72 |
| 855 | CA | CYS A | 107 | 31.320 | 7.373 | 3.829 | 1.00 | 41.84 |
| 856 | C | CYS A | 107 | 32.088 | 8.567 | 3.283 | 1.00 | 22.10 |
| 857 | 0 | CYS A | 107 | 31.791 | 9.699 | 3.617 | 1.00 | 21.70 |
| 858 | CB | CYS A | 107 | 29.941 | 7.816 | 4.309 | 1.00 | 53.08 |
| 859 | SG | CYS A | 107 | 28.736 | 8.075 | 2.959 | 1.00 | 81.46 |
| 860 | N | HIS A | 108 | 33.097 | 8.301 | 2.461 | 1.00 | 28.20 |
| 861 | CA | HIS A | 108 | 33.951 | 9.338 | 1.869 | 1.00 | 34.09 |
| 862 | CB | HIS A | 108 | 35.203 | 8.680 | 1.301 | 1.00 | 40.04 |
| 863 | CG | HIS A | 108 | 36.204 | 9.643 | 0.762 | 1.00 | 35.83 |
| 864 | CD2 | HIS A | 108 | 36.947 | 9.629 | −0.371 | 1.00 | 36.86 |
| 865 | ND1 | HIS A | 108 | 36.593 | 10.766 | 1.460 | 1.00 | 36.99 |
| 866 | CE1 | HIS A | 108 | 37.536 | 11.397 | 0.779 | 1.00 | 45.07 |
| 867 | NE2 | HIS A | 108 | 37.767 | 10.725 | −0.334 | 1.00 | 23.01 |
| 868 | C | HIS A | 108 | 33.251 | 10.165 | 0.774 | 1.00 | 42.71 |
| 869 | O | HIS A | 108 | 32.719 | 9.638 | −0.199 | 1.00 | 37.25 |
| 870 | N | GLY A | 109 | 33.269 | 11.480 | 0.946 | 1.00 | 49.87 |
| 871 | CA | GLY A | 109 | 32.623 | 12.365 | −0.006 | 1.00 | 29.45 |
| 872 | C | GLY A | 109 | 33.537 | 12.894 | −1.088 | 1.00 | 26.33 |
| 873 | O | GLY A | 109 | 34.721 | 13.111 | −0.879 | 1.00 | 19.36 |
| 874 | N | TRP A | 110 | 32.939 | 13.111 | −2.263 | 1.00 | 35.74 |
| 875 | CA | TRP A | 110 | 33.697 | 13.598 | −3.404 | 1.00 | 32.65 |
| 876 | CB | TRP A | 110 | 32.731 | 13.903 | −4.564 | 1.00 | 20.29 |
| 877 | CG | TRP A | 110 | 33.447 | 14.390 | −5.792 | 1.00 | 22.58 |
| 878 | CD2 | TRP A | 110 | 34.199 | 13.610 | −6.731 | 1.00 | 26.60 |
| 879 | CE2 | TRP A | 110 | 34.759 | 14.506 | −7.667 | 1.00 | 16.42 |
| 880 | CE3 | TRP A | 110 | 34.452 | 12.242 | −6.864 | 1.00 | 20.97 |
| 881 | CD1 | TRP A | 110 | 33.564 | 15.673 | −6.187 | 1.00 | 16.06 |
| 882 | NE1 | TRP A | 110 | 34.350 | 15.760 | −7.312 | 1.00 | 29.92 |
| 883 | CZ2 | TRP A | 110 | 35.572 | 14.088 | −8.734 | 1.00 | 11.79 |
| 884 | CZ3 | TRP A | 110 | 35.260 | 11.817 | −7.931 | 1.00 | 46.44 |
| 885 | CH2 | TRP A | 110 | 35.812 | 12.748 | −8.853 | 1.00 | 19.66 |
| 836 | C | TRP A | 110 | 34.565 | 14.791 | −3.047 | 1.00 | 29.32 |
| 887 | O | TRP A | 110 | 34.162 | 15.708 | −2.335 | 1.00 | 21.14 |
| 888 | N | ARG A | 111 | 35.814 | 14.736 | −3.559 | 1.00 | 32.25 |
| 889 | CA | ARG A | 111 | 36.819 | 15.806 | −3.372 | 1.00 | 51.05 |
| 890 | CB | ARG A | 111 | 36.294 | 17.122 | −3.911 | 1.00 | 55.38 |
| 891 | CG | ARG A | 111 | 36.335 | 17.249 | −5.405 | 1.00 | 82.45 |
| 892 | CD | ARG A | 111 | 37.728 | 16.914 | −5.919 | 1.00 | 117.48 |
| 893 | NE | ARG A | 111 | 37.701 | 16.807 | −7.377 | 1.00 | 137.24 |
| 894 | CZ | ARG A | 111 | 38.779 | 16.716 | −8.149 | 1.00 | 143.04 |
| 895 | NH1 | ARG A | 111 | 39.988 | 16.721 | −7.601 | 1.00 | 142.25 |
| 896 | NH2 | ARG A | 111 | 38.650 | 16.625 | −9.468 | 1.00 | 146.91 |
| 897 | C | ARG A | 111 | 37.110 | 16.070 | −1.919 | 1.00 | 61.58 |
| 898 | 0 | ARG A | 111 | 37.239 | 17.225 | −1.504 | 1.00 | 77.35 |
| 899 | N | ASN A | 112 | 37.217 | 15.017 | −1.101 | 1.00 | 59.89 |
| 900 | CA | ASN A | 112 | 37.433 | 15.116 | 0.363 | 1.00 | 47.67 |
| 901 | CB | ASN A | 112 | 38.892 | 15.480 | 0.648 | 1.00 | 68.19 |
| 902 | CG | ASN A | 112 | 39.700 | 14.277 | 1.106 | 1.00 | 77.01 |
| 903 | OD1 | ASN A | 112 | 39.474 | 13.725 | 2.188 | 1.00 | 77.39 |
| 904 | ND2 | ASN A | 112 | 40.669 | 13.866 | 0.282 | 1.00 | 78.90 |
| 905 | C | ASN A | 112 | 36.433 | 16.068 | 0.984 | 1.00 | 28.46 |
| 906 | O | ASN A | 112 | 36.705 | 16.745 | 1.990 | 1.00 | 47.00 |
| 907 | N | TRP A | 113 | 35.263 | 16.090 | 0.367 | 1.00 | 35.39 |
| 908 | CA | TRP A | 113 | 34.201 | 16.977 | 0.819 | 1.00 | 51.08 |
| 909 | CB | TRP A | 113 | 33.208 | 17.136 | −0.253 | 1.00 | 64.97 |
| 910 | CG | TRP A | 113 | 32.539 | 18.373 | 0.147 | 1.00 | 81.55 |
| 911 | CD2 | TRP A | 113 | 32.917 | 19.678 | −0.301 | 1.00 | 74.85 |
| 912 | CE2 | TRP A | 113 | 32.014 | 20.570 | 0.303 | 1.00 | 85.26 |
| 913 | CE3 | TRP A | 113 | 33.902 | 20.163 | −1.177 | 1.00 | 61.05 |
| 914 | CD1 | TRP A | 113 | 31.496 | 18.519 | 0.997 | 1.00 | 83.43 |
| 915 | NE1 | TRP A | 113 | 31.158 | 19.856 | 1.104 | 1.00 | 83.67 |
| 916 | CZ2 | TRP A | 113 | 32.073 | 21.949 | 0.055 | 1.00 | 96.42 |
| 917 | CZ3 | TRP A | 113 | 33.958 | 21.536 | −1.420 | 1.00 | 72.53 |
| 918 | CH2 | TRP A | 113 | 33.046 | 22.396 | −0.803 | 1.00 | 92.25 |
| 919 | C | TRP A | 113 | 33.482 | 16.590 | 2.066 | 1.00 | 46.75 |
| 920 | O | TRP A | 113 | 33.409 | 15.393 | 2.379 | 1.00 | 35.50 |
| 921 | N | ASP A | 114 | 32.921 | 17.551 | 2.792 | 1.00 | 63.72 |
| 922 | CA | ASP A | 114 | 32.243 | 17.173 | 4.025 | 1.00 | 64.61 |
| 923 | CB | ASP A | 114 | 32.177 | 18.380 | 4.943 | 1.00 | 86.45 |
| 924 | CG | ASP A | 114 | 33.506 | 18.737 | 5.602 | 1.00 | 98.41 |
| 925 | OD1 | ASP A | 114 | 34.402 | 17.872 | 5.707 | 1.00 | 84.34 |
| 926 | OD2 | ASP A | 114 | 33.641 | 19.918 | 6.023 | 1.00 | 109.32 |
| 927 | C | ASP A | 114 | 30.871 | 16.558 | 3.829 | 1.00 | 61.40 |
| 928 | O | ASP A | 114 | 30.070 | 17.044 | 3.039 | 1.00 | 81.69 |
| 929 | N | VAL A | 115 | 30.569 | 15.463 | 4.556 | 1.00 | 39.40 |
| 930 | CA | VAL A | 115 | 29.265 | 14.772 | 4.391 | 1.00 | 37.88 |
| 931 | CB | VAL A | 115 | 29.491 | 13.274 | 4.126 | 1.00 | 10.52 |
| 932 | CG1 | VAL A | 115 | 28.209 | 12.621 | 3.617 | 1.00 | 5.47 |
| 933 | CG2 | VAL A | 115 | 30.635 | 13.071 | 3.147 | 1.00 | 9.13 |
| 934 | C | VAL A | 115 | 28.364 | 14.928 | 5.596 | 1.00 | 28.56 |
| 935 | O | VAL A | 115 | 28.807 | 14.733 | 6.740 | 1.00 | 19.72 |
| 936 | N | TYR A | 116 | 27.101 | 15.273 | 5.384 | 1.00 | 37.21 |
| 937 | CA | TYR A | 116 | 26.192 | 15.428 | 6.517 | 1.00 | 46.14 |
| 938 | CB | TYR A | 116 | 25.652 | 16.857 | 6.574 | 1.00 | 61.34 |
| 939 | CG | TYR A | 116 | 26.725 | 17.921 | 6.689 | 1.00 | 76.00 |
| 940 | CD1 | TYR A | 116 | 27.203 | 18.582 | 5.557 | 1.00 | 87.94 |
| 941 | CE1 | TYR A | 116 | 28.190 | 19.572 | 5.658 | 1.00 | 91.76 |
| 942 | CD2 | TYR A | 116 | 27.264 | 18.269 | 7.931 | 1.00 | 84.35 |
| 943 | CE2 | TYR A | 116 | 28.255 | 19.255 | 8.041 | 1.00 | 88.13 |
| 944 | CZ | TYR A | 116 | 28.709 | 19.897 | 6.899 | 1.00 | 83.79 |
| 945 | OH | TYR A | 116 | 29.683 | 20.859 | 6.988 | 1.00 | 79.96 |
| 946 | C | TYR A | 116 | 25.022 | 14.449 | 6.489 | 1.00 | 48.03 |
| 947 | O | TYR A | 116 | 24.764 | 13.805 | 5.461 | 1.00 | 47.24 |
| 948 | N | LYS A | 117 | 24.313 | 14.360 | 7.618 | 1.00 | 49.04 |
| 949 | CA | LYS A | 117 | 23.175 | 13.455 | 7.754 | 1.00 | 62.64 |
| 950 | CB | LYS A | 117 | 21.940 | 14.043 | 7.066 | 1.00 | 84.33 |
| 951 | CG | LYS A | 117 | 21.382 | 15.296 | 7.713 | 1.00 | 103.44 |
| 952 | CD | LYS A | 117 | 20.779 | 15.016 | 9.087 | 1.00 | 122.93 |
| 953 | CE | LYS A | 117 | 20.018 | 16.235 | 9.606 | 1.00 | 125.37 |
| 954 | NZ | LYS A | 117 | 19.408 | 16.006 | 10.942 | 1.00 | 122.53 |
| 955 | C | LYS A | 117 | 23.543 | 12.117 | 7.115 | 1.00 | 59.71 |
| 956 | 0 | LYS A | 117 | 22.858 | 11.626 | 6.207 | 1.00 | 73.62 |
| 957 | N | VAL A | 118 | 24.636 | 11.532 | 7.588 | 1.00 | 36.40 |
| 958 | CA | VAL A | 118 | 25.107 | 10.262 | 7.057 | 1.00 | 27.92 |
| 959 | CB | VAL A | 118 | 26.612 | 10.090 | 7.262 | 1.00 | 4.59 |
| 960 | CG1 | VAL A | 118 | 27.021 | 8.714 | 6.845 | 1.00 | 4.59 |
| 961 | CG2 | VAL A | 118 | 27.369 | 11.124 | 6.440 | 1.00 | 27.69 |
| 962 | C | VAL A | 118 | 24.421 | 9.071 | 7.685 | 1.00 | 21.65 |
| 963 | O | VAL A | 118 | 24.371 | 8.943 | 8.897 | 1.00 | 23.38 |
| 964 | N | ILE A | 119 | 23.882 | 8.205 | 6.843 | 1.00 | 29.44 |
| 965 | CA | ILE A | 119 | 23.222 | 7.008 | 7.313 | 1.00 | 32.32 |
| 966 | CB | ILE A | 119 | 21.749 | 7.038 | 7.026 | 1.00 | 15.68 |
| 967 | CG2 | ILE A | 119 | 21.120 | 5.711 | 7.453 | 1.00 | 9.94 |
| 968 | CG1 | ILE A | 119 | 21.116 | 8.205 | 7.779 | 1.00 | 16.41 |
| 969 | CD1 | ILE A | 119 | 19.568 | 8.203 | 7.722 | 1.00 | 28.41 |
| 970 | C | ILE A | 119 | 23.817 | 5.818 | 6.605 | 1.00 | 41.89 |
| 971 | 0 | ILE A | 119 | 24.159 | 5.901 | 5.424 | 1.00 | 51.23 |
| 972 | N | TYR A | 120 | 23.973 | 4.719 | 7.330 | 1.00 | 34.78 |
| 973 | CA | TYR A | 120 | 24.530 | 3.531 | 6.720 | 1.00 | 31.98 |
| 974 | CB | TYR A | 120 | 25.732 | 3.013 | 7.510 | 1.00 | 6.64 |
| 975 | CG | TYR A | 120 | 26.965 | 3.845 | 7.362 | 1.00 | 4.59 |
| 976 | CD1 | TYR A | 120 | 27.192 | 4.931 | 8.175 | 1.00 | 7.90 |
| 977 | CE1 | TYR A | 120 | 28.354 | 5.687 | 8.055 | 1.00 | 5.34 |
| 978 | CD2 | TYR A | 120 | 27.912 | 3.527 | 6.413 | 1.00 | 8.49 |
| 979 | CE2 | TYR A | 120 | 29.085 | 4.271 | 6.270 | 1.00 | 23.20 |
| 980 | CZ | TYR A | 120 | 29.304 | 5.353 | 7.090 | 1.00 | 24.78 |
| 981 | OH | TYR A | 120 | 30.467 | 6.077 | 6.927 | 1.00 | 41.99 |
| 982 | C | TYR A | 120 | 23.454 | 2.476 | 6.672 | 1.00 | 39.78 |
| 983 | O | TYR A | 120 | 22.664 | 2.339 | 7.614 | 1.00 | 25.06 |
| 984 | N | TYR A | 121 | 23.406 | 1.748 | 5.564 | 1.00 | 40.70 |
| 985 | CA | TYR A | 121 | 22.421 | 0.694 | 5.421 | 1.00 | 43.93 |
| 986 | CB | TYR A | 121 | 21.497 | 0.982 | 4.248 | 1.00 | 37.43 |
| 987 | CG | TYR A | 121 | 20.739 | 2.288 | 4.367 | 1.00 | 49.95 |
| 988 | CD1 | TYR A | 121 | 21.387 | 3.511 | 4.161 | 1.00 | 49.22 |
| 989 | CE1 | TYR A | 121 | 20.691 | 4.723 | 4.262 | 1.00 | 56.51 |
| 990 | CD2 | TYR A | 121 | 19.369 | 2.303 | 4.678 | 1.00 | 25.20 |
| 991 | CE2 | TYR A | 121 | 18.664 | 3.494 | 4.783 | 1.00 | 32.17 |
| 992 | CZ | TYR A | 121 | 19.326 | 4.709 | 4.572 | 1.00 | 58.52 |
| 993 | OH | TYR A | 121 | 18.632 | 5.904 | 4.685 | 1.00 | 47.56 |
| 994 | C | TYR A | 121 | 23.095 | −0.650 | 5.206 | 1.00 | 57.82 |
| 995 | O | TYR A | 121 | 23.997 | −0.778 | 4.341 | 1.00 | 56.35 |
| 996 | N | LYS A | 122 | 22.677 | −1.636 | 6.003 | 1.00 | 50.06 |
| 997 | CA | LYS A | 122 | 23.216 | −2.978 | 5.881 | 1.00 | 54.50 |
| 998 | CB | LYS A | 122 | 23.790 | −3.487 | 7.209 | 1.00 | 66.15 |
| 999 | CG | LYS A | 122 | 24.742 | −4.667 | 7.027 | 1.00 | 79.15 |
| 1000 | CD | LYS A | 122 | 24.992 | −5.443 | 8.315 | 1.00 | 81.56 |
| 1001 | CE | LYS A | 122 | 23.821 | −6.355 | 8.653 | 1.00 | 78.05 |
| 1002 | NZ | LYS A | 122 | 24.119 | −7.213 | 9.829 | 1.00 | 89.70 |
| 1003 | C | LYS A | 122 | 22.040 | −3.841 | 5.463 | 1.00 | 56.73 |
| 1004 | O | LYS A | 122 | 21.202 | −4.210 | 6.289 | 1.00 | 47.59 |
| 1005 | N | ASP A | 123 | 21.978 | −4.138 | 4.168 | 1.00 | 66.50 |
| 1006 | CA | ASP A | 123 | 20.911 | −4.951 | 3.599 | 1.00 | 73.31 |
| 1007 | CB | ASP A | 123 | 20.768 | −6.270 | 4.365 | 1.00 | 80.64 |
| 1008 | CG | ASP A | 123 | 21.977 | −7.186 | 4.190 | 1.00 | 94.21 |
| 1009 | OD1 | ASP A | 123 | 22.334 | −7.499 | 3.031 | 1.00 | 103.57 |
| 1010 | OD2 | ASP A | 123 | 22.568 | −7.600 | 5.214 | 1.00 | 97.62 |
| 1011 | C | ASP A | 123 | 19.591 | −4.203 | 3.608 | 1.00 | 74.17 |
| 1012 | O | ASP A | 123 | 18.616 | −4.644 | 4.220 | 1.00 | 66.73 |
| 1013 | N | GLY A | 124 | 19.571 | −3.062 | 2.928 | 1.00 | 75.26 |
| 1014 | CA | GLY A | 124 | 18.362 | −2.265 | 2.851 | 1.00 | 79.44 |
| 1015 | C | GLY A | 124 | 17.954 | −1.637 | 4.166 | 1.00 | 80.57 |
| 1016 | 0 | GLY A | 124 | 17.323 | −0.580 | 4.186 | 1.00 | 91.15 |
| 1017 | N | GLU A | 125 | 18.301 | −2.295 | 5.268 | 1.00 | 74.63 |
| 1018 | CA | GLU A | 125 | 17.981 | −1.786 | 6.594 | 1.00 | 61.74 |
| 1019 | CB | GLU A | 125 | 18.066 | −2.914 | 7.616 | 1.00 | 79.17 |
| 1020 | CG | GLU A | 125 | 17.092 | −4.054 | 7.365 | 1.00 | 98.38 |
| 1021 | CD | GLU A | 125 | 15.642 | −3.619 | 7.478 | 1.00 | 106.78 |
| 1022 | OE1 | GLU A | 125 | 15.235 | −3.167 | 8.570 | 1.00 | 118.36 |
| 1023 | OE2 | GLU A | 125 | 14.907 | −3.728 | 6.475 | 1.00 | 110.58 |
| 1024 | C | GLU A | 125 | 18.947 | −0.660 | 6.981 | 1.00 | 58.10 |
| 1025 | O | GLU A | 125 | 20.161 | −0.697 | 6.664 | 1.00 | 30.38 |
| 1026 | N | ALA A | 126 | 18.399 | 0.343 | 7.662 | 1.00 | 50.55 |
| 1027 | CA | ALA A | 126 | 19.185 | 1.490 | 8.088 | 1.00 | 52.07 |
| 1028 | CB | ALA A | 126 | 18.322 | 2.732 | 8.098 | 1.00 | 53.61 |
| 1029 | C | ALA A | 126 | 19.795 | 1.272 | 9.461 | 1.00 | 45.73 |
| 1030 | O | ALA A | 126 | 19.077 | 1.194 | 10.459 | 1.00 | 48.02 |
| 1031 | N | LEU A | 127 | 21.122 | 1.179 | 9.491 | 1.00 | 34.72 |
| 1032 | CA | LEU A | 127 | 21.895 | 0.979 | 10.720 | 1.00 | 30.29 |
| 1033 | CB | LEU A | 127 | 23.358 | 0.751 | 10.350 | 1.00 | 15.93 |
| 1034 | CG | LEU A | 127 | 23.473 | −0.487 | 9.465 | 1.00 | 4.91 |
| 1035 | CD1 | LEU A | 127 | 24.904 | −0.710 | 9.010 | 1.00 | 4.59 |
| 1036 | CD2 | LEU A | 127 | 22.935 | −1.692 | 10.260 | 1.00 | 16.61 |
| 1037 | C | LEU A | 127 | 21.782 | 2.175 | 11.660 | 1.00 | 21.97 |
| 1038 | O | LEU A | 127 | 22.724 | 2.962 | 11.801 | 1.00 | 23.95 |
| 1039 | N | LYS A | 128 | 20.635 | 2.305 | 12.315 | 1.00 | 19.75 |
| 1040 | CA | LYS A | 128 | 20.391 | 3.427 | 13.205 | 1.00 | 29.37 |
| 1041 | CB | LYS A | 128 | 19.091 | 3.206 | 13.951 | 1.00 | 16.78 |
| 1042 | CG | LYS A | 128 | 17.911 | 2.993 | 13.019 | 1.00 | 47.56 |
| 1043 | CD | LYS A | 128 | 16.603 | 2.817 | 13.777 | 1.00 | 56.74 |
| 1044 | CE | LYS A | 128 | 15.492 | 2.380 | 12.824 | 1.00 | 58.68 |
| 1045 | NZ | LYS A | 128 | 14.183 | 2.220 | 13.519 | 1.00 | 55.73 |
| 1046 | C | LYS A | 128 | 21.529 | 3.647 | 14.187 | 1.00 | 40.15 |
| 1047 | O | LYS A | 128 | 22.289 | 4.599 | 14.064 | 1.00 | 53.09 |
| 1048 | N | TYR A | 129 | 21.647 | 2.757 | 15.159 | 1.00 | 36.41 |
| 1049 | CA | TYR A | 129 | 22.707 | 2.860 | 16.144 | 1.00 | 37.61 |
| 1050 | CB | TYR A | 129 | 23.134 | 1.465 | 16.629 | 1.00 | 55.67 |
| 1051 | CG | TYR A | 129 | 22.011 | 0.668 | 17.256 | 1.00 | 69.76 |
| 1052 | CD1 | TYR A | 129 | 21.225 | −0.184 | 16.491 | 1.00 | 84.41 |
| 1053 | CE1 | TYR A | 129 | 20.152 | −0.868 | 17.051 | 1.00 | 80.71 |
| 1054 | CD2 | TYR A | 129 | 21.699 | 0.813 | 18.604 | 1.00 | 78.34 |
| 1055 | CE2 | TYR A | 129 | 20.632 | 0.136 | 19.172 | 1.00 | 85.65 |
| 1056 | CZ | TYR A | 129 | 19.860 | −0.702 | 18.391 | 1.00 | 81.90 |
| 1057 | OH | TYR A | 129 | 18.794 | −1.369 | 18.952 | 1.00 | 78.93 |
| 1058 | C | TYR A | 129 | 23.938 | 3.635 | 15.664 | 1.00 | 41.39 |
| 1059 | O | TYR A | 129 | 24.414 | 4.513 | 16.379 | 1.00 | 73.88 |
| 1060 | N | TRP A | 130 | 24.451 | 3.321 | 14.475 | 1.00 | 21.26 |
| 1061 | CA | TRP A | 130 | 25.630 | 4.021 | 13.983 | 1.00 | 55.41 |
| 1062 | CB | TRP A | 130 | 26.227 | 3.270 | 12.798 | 1.00 | 66.63 |
| 1063 | CG | TRP A | 130 | 26.560 | 1.867 | 13.090 | 1.00 | 78.03 |
| 1064 | CD2 | TRP A | 130 | 27.747 | 1.387 | 13.717 | 1.00 | 80.17 |
| 1065 | CE2 | TRP A | 130 | 27.652 | −0.021 | 13.762 | 1.00 | 96.27 |
| 1066 | CE3 | TRP A | 130 | 28.889 | 2.009 | 14.244 | 1.00 | 63.46 |
| 1067 | CD1 | TRP A | 130 | 25.807 | 0.770 | 12.791 | 1.00 | 95.81 |
| 1068 | NE1 | TRP A | 130 | 26.456 | −0.371 | 13.189 | 1.00 | 93.15 |
| 1069 | CZ2 | TRP A | 130 | 28.657 | −0.822 | 14.314 | 1.00 | 106.12 |
| 1070 | CZ3 | TRP A | 130 | 29.888 | 1.215 | 14.792 | 1.00 | 70.51 |
| 1071 | CH2 | TRP A | 130 | 29.764 | −0.188 | 14.823 | 1.00 | 97.78 |
| 1072 | C | TRP A | 130 | 25.397 | 5.477 | 13.569 | 1.00 | 69.95 |
| 1073 | O | TRP A | 130 | 26.292 | 6.113 | 13.027 | 1.00 | 86.40 |
| 1074 | N | TYR A | 131 | 24.215 | 6.013 | 13.841 | 1.00 | 75.40 |
| 1075 | CA | TYR A | 131 | 23.916 | 7.387 | 13.449 | 1.00 | 97.14 |
| 1076 | CB | TYR A | 131 | 22.479 | 7.739 | 13.825 | 1.00 | 111.94 |
| 1077 | CG | TYR A | 131 | 21.976 | 8.995 | 13.151 | 1.00 | 148.46 |
| 1078 | CD1 | TYR A | 131 | 21.452 | 8.958 | 11.863 | 1.00 | 154.63 |
| 1079 | CE1 | TYR A | 131 | 20.988 | 10.111 | 11.242 | 1.00 | 166.01 |
| 1080 | CD2 | TYR A | 131 | 22.025 | 10.223 | 13.800 | 1.00 | 162.09 |
| 1081 | CE2 | TYR A | 131 | 21.566 | 11.383 | 13.187 | 1.00 | 169.95 |
| 1082 | CZ | TYR A | 131 | 21.046 | 11.320 | 11.909 | 1.00 | 172.27 |
| 1083 | OH | TYR A | 131 | 20.578 | 12.465 | 11.306 | 1.00 | 171.20 |
| 1084 | C | TYR A | 131 | 24.854 | 8.424 | 14.048 | 1.00 | 107.44 |
| 1085 | O | TYR A | 131 | 25.432 | 9.238 | 13.326 | 1.00 | 112.84 |
| 1086 | N | GLU A | 132 | 24.991 | 8.410 | 15.368 | 1.00 | 111.22 |
| 1087 | CA | GLU A | 132 | 25.857 | 9.362 | 16.047 | 1.00 | 114.10 |
| 1088 | CB | GLU A | 132 | 26.071 | 8.954 | 17.505 | 1.00 | 121.12 |
| 1089 | CG | GLU A | 132 | 24.802 | 8.888 | 18.342 | 1.00 | 146.81 |
| 1090 | CD | GLU A | 132 | 25.067 | 8.459 | 19.774 | 1.00 | 156.79 |
| 1091 | OE1 | GLU A | 132 | 26.243 | 8.195 | 20.104 | 1.00 | 168.06 |
| 1092 | OE2 | GLU A | 132 | 24.100 | 8.386 | 20.565 | 1.00 | 164.60 |
| 1093 | C | GLU A | 132 | 27.212 | 9.442 | 15.359 | 1.00 | 112.41 |
| 1094 | O | GLU A | 132 | 27.715 | 10.523 | 15.090 | 1.00 | 126.08 |
| 1095 | N | ASN A | 133 | 27.792 | 8.286 | 15.065 | 1.00 | 107.15 |
| 1096 | CA | ASN A | 133 | 29.101 | 8.246 | 14.436 | 1.00 | 110.99 |
| 1097 | CB | ASN A | 133 | 29.828 | 6.972 | 14.874 | 1.00 | 132.56 |
| 1098 | CG | ASN A | 133 | 29.903 | 6.843 | 16.381 | 1.00 | 152.00 |
| 1099 | OD1 | ASN A | 133 | 30.195 | 7.810 | 17.084 | 1.00 | 156.21 |
| 1100 | ND2 | ASN A | 133 | 29.636 | 5.653 | 16.882 | 1.00 | 164.20 |
| 1101 | C | ASN A | 133 | 29.071 | 8.366 | 12.913 | 1.00 | 103.70 |
| 1102 | O | ASN A | 133 | 28.344 | 7.653 | 12.236 | 1.00 | 103.53 |
| 1103 | N | HIS A | 134 | 29.885 | 9.274 | 12.388 | 1.00 | 108.74 |
| 1104 | CA | HIS A | 134 | 29.987 | 9.520 | 10.954 | 1.00 | 130.31 |
| 1105 | CB | HIS A | 134 | 30.880 | 10.729 | 10.703 | 1.00 | 148.82 |
| 1106 | CG | HIS A | 134 | 30.457 | 11.949 | 11.454 | 1.00 | 166.98 |
| 1107 | CD2 | HIS A | 134 | 31.030 | 12.601 | 12.493 | 1.00 | 171.29 |
| 1108 | ND1 | HIS A | 134 | 29.283 | 12.620 | 11.183 | 1.00 | 179.56 |
| 1109 | CE1 | HIS A | 134 | 29.153 | 13.629 | 12.026 | 1.00 | 185.16 |
| 1110 | NE2 | HIS A | 134 | 30.196 | 13.641 | 12.829 | 1.00 | 183.14 |
| 1111 | C | HIS A | 134 | 30.572 | 8.320 | 10.242 | 1.00 | 130.17 |
| 1112 | 0 | HIS A | 134 | 30.194 | 8.013 | 9.120 | 1.00 | 144.97 |
| 1113 | N | ASN A | 135 | 31.506 | 7.668 | 10.891 | 1.00 | 115.50 |
| 1114 | CA | ASN A | 135 | 32.133 | 6.470 | 10.351 | 1.00 | 106.66 |
| 1115 | CB | ASN A | 135 | 33.641 | 6.677 | 10.201 | 1.00 | 111.41 |
| 1116 | CG | ASN A | 135 | 34.357 | 6.732 | 11.535 | 1.00 | 121.66 |
| 1117 | OD1 | ASN A | 135 | 33.734 | 6.607 | 12.590 | 1.00 | 137.22 |
| 1118 | ND2 | ASN A | 135 | 35.671 | 6.919 | 11.493 | 1.00 | 131.58 |
| 1119 | C | ASN A | 135 | 31.848 | 5.256 | 11.226 | 1.00 | 98.67 |
| 1120 | O | ASN A | 135 | 31.560 | 5.429 | 12.414 | 1.00 | 95.00 |
| 1121 | N | ILE A | 136 | 31.914 | 4.084 | 10.657 | 1.00 | 84.56 |
| 1122 | CA | !LE A | 136 | 31.590 | 2.901 | 11.441 | 1.00 | 68.70 |
| 1123 | CB | ILE A | 136 | 30.615 | 2.005 | 10.689 | 1.00 | 70.57 |
| 1124 | CG2 | ILE A | 136 | 30.242 | 0.824 | 11.556 | 1.00 | 57.24 |
| 1125 | CG1 | ILE A | 136 | 29.371 | 2.809 | 10.308 | 1.00 | 76.41 |
| 1126 | CD1 | ILE A | 136 | 28.315 | 1.994 | 9.607 | 1.00 | 81.35 |
| 1127 | C | ILE A | 136 | 32.825 | 2.108 | 11.771 | 1.00 | 67.79 |
| 1128 | 0 | ILE A | 136 | 33.484 | 1.575 | 10.879 | 1.00 | 75.89 |
| 1129 | N | SER A | 137 | 33.153 | 2.051 | 13.057 | 1.00 | 64.01 |
| 1130 | CA | SER A | 137 | 34.327 | 1.314 | 13.512 | 1.00 | 50.08 |
| 1131 | CB | SER A | 137 | 35.240 | 2.228 | 14.319 | 1.00 | 42.18 |
| 1132 | OG | SER A | 137 | 36.435 | 1.547 | 14.654 | 1.00 | 74.65 |
| 1133 | C | SER A | 137 | 33.882 | 0.137 | 14.366 | 1.00 | 40.13 |
| 1134 | O | SER A | 137 | 33.257 | 0.331 | 15.402 | 1.00 | 31.91 |
| 1135 | N | ILE A | 138 | 34.172 | −1.079 | 13.915 | 1.00 | 47.57 |
| 1136 | CA | ILE A | 138 | 33.767 | −2.272 | 14.646 | 1.00 | 55.59 |
| 1137 | CB | ILE A | 138 | 32.849 | −3.173 | 13.801 | 1.00 | 45.34 |
| 1138 | CG2 | ILE A | 138 | 32.360 | −4.317 | 14.646 | 1.00 | 52.83 |
| 1139 | CG1 | ILE A | 138 | 31.640 | −2.378 | 13.310 | 1.00 | 55.63 |
| 1140 | CD1 | ILE A | 138 | 30.680 | −3.166 | 12.457 | 1.00 | 59.56 |
| 1141 | C | ILE A | 138 | 35.014 | −3.039 | 15.023 | 1.00 | 69.36 |
| 1142 | O | ILE A | 138 | 35.542 | −3.824 | 14.243 | 1.00 | 86.80 |
| 1143 | N | THR A | 139 | 35.481 | −2.793 | 16.238 | 1.00 | 80.29 |
| 1144 | CA | THR A | 139 | 36.687 | −3.412 | 16.765 | 1.00 | 71.05 |
| 1145 | CB | THR A | 139 | 36.824 | −3.094 | 18.243 | 1.00 | 65.21 |
| 1146 | OG1 | THR A | 139 | 35.584 | −3.396 | 18.894 | 1.00 | 63.19 |
| 1147 | CG2 | THR A | 139 | 37.152 | −1.610 | 18.429 | 1.00 | 37.67 |
| 1148 | C | THR A | 139 | 36.727 | −4.906 | 16.577 | 1.00 | 73.17 |
| 1149 | O | THR A | 139 | 37.633 | −5.439 | 15.927 | 1.00 | 72.09 |
| 1150 | N | ASN A | 140 | 35.731 | −5.585 | 17.126 | 1.00 | 71.99 |
| 1151 | CA | ASN A | 140 | 35.689 | −7.021 | 17.012 | 1.00 | 74.50 |
| 1152 | CB | ASN A | 140 | 35.704 | −7.615 | 18.427 | 1.00 | 83.94 |
| 1153 | CG | ASN A | 140 | 37.023 | −8.292 | 18.747 | 1.00 | 102.78 |
| 1154 | OD1 | ASN A | 140 | 37.329 | −9.296 | 18.114 | 1.00 | 129.44 |
| 1155 | ND2 | ASN A | 140 | 37.842 | −7.797 | 19.666 | 1.00 | 102.21 |
| 1156 | C | ASN A | 140 | 34.509 | −7.489 | 16.134 | 1.00 | 66.79 |
| 1157 | O | ASN A | 140 | 33.351 | −7.495 | 16.568 | 1.00 | 67.94 |
| 1158 | N | ALA A | 141 | 34.834 | −7.874 | 14.888 | 1.00 | 51.28 |
| 1159 | CA | ALA A | 141 | 33.851 | −8.305 | 13.837 | 1.00 | 50.10 |
| 1160 | CB | ALA A | 141 | 34.536 | −8.441 | 12.546 | 1.00 | 44.66 |
| 1161 | C | ALA A | 141 | 33.037 | −9.568 | 14.167 | 1.00 | 57.39 |
| 1162 | 0 | ALA A | 141 | 33.395 | −10.379 | 15.013 | 1.00 | 76.44 |
| 1163 | N | THR A | 142 | 31.940 | −9.721 | 13.424 | 1.00 | 63.44 |
| 1164 | CA | THR A | 142 | 31.029 | −10.864 | 13.548 | 1.00 | 68.80 |
| 1165 | CB | THR A | 142 | 29.830 | −10.522 | 14.429 | 1.00 | 56.28 |
| 1166 | OG1 | THR A | 142 | 30.292 | −9.914 | 15.637 | 1.00 | 69.20 |
| 1167 | CG2 | THR A | 142 | 29.038 | −11.771 | 14.756 | 1.00 | 63.69 |
| 1168 | C | THR A | 142 | 30.476 | −11.284 | 12.186 | 1.00 | 77.84 |
| 1169 | O | THR A | 142 | 30.350 | −10.467 | 11.275 | 1.00 | 84.69 |
| 1170 | N | VAL A | 143 | 30.133 | −12.562 | 12.053 | 1.00 | 80.43 |
| 1171 | CA | VAL A | 143 | 29.594 | −13.071 | 10.800 | 1.00 | 75.62 |
| 1172 | CB | VAL A | 143 | 29.347 | −14.600 | 10.870 | 1.00 | 62.57 |
| 1173 | CG1 | VAL A | 143 | 28.297 | −14.907 | 11.934 | 1.00 | 69.07 |
| 1174 | CG2 | VAL A | 143 | 28.903 | −15.123 | 9.516 | 1.00 | 78.52 |
| 1175 | C | VAL A | 143 | 28.282 | −12.361 | 10.472 | 1.00 | 73.71 |
| 1176 | 0 | VAL A | 143 | 27.893 | −12.285 | 9.307 | 1.00 | 85.08 |
| 1177 | N | GLU A | 144 | 27.593 | −11.846 | 11.491 | 1.00 | 65.50 |
| 1178 | CA | GLU A | 144 | 26.347 | −11.130 | 11.245 | 1.00 | 78.86 |
| 1179 | CB | GLU A | 144 | 25.554 | −10.974 | 12.544 | 1.00 | 88.26 |
| 1180 | CG | GLU A | 144 | 24.140 | −10.450 | 12.348 | 1.00 | 123.20 |
| 1181 | CD | GLU A | 144 | 23.389 | −10.299 | 13.656 | 1.00 | 142.61 |
| 1182 | OE1 | GLU A | 144 | 23.977 | −10.598 | 14.717 | 1.00 | 141.13 |
| 1183 | OE2 | GLU A | 144 | 22.212 | −9.883 | 13.620 | 1.00 | 155.89 |
| 1184 | C | GLU A | 144 | 26.611 | −9.767 | 10.616 | 1.00 | 84.50 |
| 1185 | 0 | GLU A | 144 | 25.793 | −9.241 | 9.855 | 1.00 | 86.40 |
| 1186 | N | ASP A | 145 | 27.764 | −9.198 | 10.943 | 1.00 | 83.52 |
| 1187 | CA | ASP A | 145 | 28.139 | −7.913 | 10.383 | 1.00 | 73.54 |
| 1188 | CB | ASP A | 145 | 29.429 | −7.402 | 11.033 | 1.00 | 81.77 |
| 1189 | CG | ASP A | 145 | 29.191 | −6.826 | 12.430 | 1.00 | 98.36 |
| 1190 | OD1 | ASP A | 145 | 28.336 | −5.914 | 12.559 | 1.00 | 102.22 |
| 1191 | OD2 | ASP A | 145 | 29.856 | −7.280 | 13.394 | 1.00 | 95.71 |
| 1192 | C | ASP A | 145 | 28.317 | −8.028 | 8.875 | 1.00 | 65.16 |
| 1193 | O | ASP A | 145 | 28.626 | −7.053 | 8.211 | 1.00 | 74.60 |
| 1194 | N | SER A | 146 | 28.112 | −9.224 | 8.337 | 1.00 | 63.10 |
| 1195 | CA | SER A | 146 | 28.246 | −9.460 | 6.906 | 1.00 | 51.02 |
| 1196 | CB | SER A | 146 | 28.403 | −10.959 | 6.619 | 1.00 | 62.55 |
| 1197 | OG | SER A | 146 | 29.671 | −11.452 | 7.030 | 1.00 | 53.04 |
| 1198 | C | SER A | 146 | 26.997 | −8.949 | 6.221 | 1.00 | 50.62 |
| 1199 | O | SER A | 146 | 25.935 | −8.865 | 6.848 | 1.00 | 44.95 |
| 1200 | N | GLY A | 147 | 27.130 | −8.609 | 4.942 | 1.00 | 48.93 |
| 1201 | CA | GLY A | 147 | 25.992 | −8.119 | 4.185 | 1.00 | 55.32 |
| 1202 | C | GLY A | 147 | 26.389 | −7.039 | 3.199 | 1.00 | 60.64 |
| 1203 | 0 | GLY A | 147 | 27.587 | −6.725 | 3.051 | 1.00 | 55.89 |
| 1204 | N | THR A | 148 | 25.396 | −6.477 | 2.510 | 1.00 | 61.14 |
| 1205 | CA | THR A | 148 | 25.665 | −5.415 | 1.540 | 1.00 | 66.29 |
| 1206 | CB | THR A | 148 | 24.735 | −5.502 | 0.318 | 1.00 | 67.47 |
| 1207 | OG1 | THR A | 148 | 23.514 | −4.813 | 0.604 | 1.00 | 89.27 |
| 1208 | CG2 | THR A | 148 | 24.414 | −6.954 | −0.006 | 1.00 | 53.85 |
| 1209 | C | THR A | 148 | 25.442 | −4.064 | 2.229 | 1.00 | 60.43 |
| 1210 | O | THR A | 148 | 24.373 | −3.805 | 2.793 | 1.00 | 48.98 |
| 1211 | N | TYR A | 149 | 26.461 | −3.214 | 2.203 | 1.00 | 56.49 |
| 1212 | CA | TYR A | 149 | 26.371 | −1.911 | 2.836 | 1.00 | 46.76 |
| 1213 | CB | TYR A | 149 | 27.600 | −1.648 | 3.726 | 1.00 | 50.51 |
| 1214 | CG | TYR A | 149 | 27.679 | −2.436 | 5.010 | 1.00 | 37.62 |
| 1215 | CD1 | TYR A | 149 | 28.197 | −3.727 | 5.030 | 1.00 | 38.64 |
| 1216 | CE1 | TYR A | 149 | 28.288 | −4.446 | 6.214 | 1.00 | 21.54 |
| 1217 | CD2 | TYR A | 149 | 27.252 | −1.882 | 6.211 | 1.00 | 27.02 |
| 1218 | CE2 | TYR A | 149 | 27.336 | −2.589 | 7.393 | 1.00 | 25.84 |
| 1219 | CZ | TYR A | 149 | 27.854 | −3.868 | 7.390 | 1.00 | 22.38 |
| 1220 | OH | TYR A | 149 | 27.923 | −4.562 | 8.573 | 1.00 | 47.89 |
| 1221 | C | TYR A | 149 | 26.329 | −0.824 | 1.775 | 1.00 | 47.29 |
| 1222 | O | TYR A | 149 | 26.626 | −1.059 | 0.590 | 1.00 | 37.99 |
| 1223 | N | TYR A | 150 | 25.962 | 0.369 | 2.225 | 1.00 | 35.54 |
| 1224 | CA | TYR A | 150 | 25.947 | 1.547 | 1.380 | 1.00 | 37.17 |
| 1225 | CB | TYR A | 150 | 25.029 | 1.341 | 0.165 | 1.00 | 20.88 |
| 1226 | CG | TYR A | 150 | 23.546 | 1.438 | 0.440 | 1.00 | 11.30 |
| 1227 | CD1 | TYR A | 150 | 22.897 | 2.667 | 0.465 | 1.00 | 12.45 |
| 1228 | CE1 | TYR A | 150 | 21.518 | 2.751 | 0.712 | 1.00 | 32.37 |
| 1229 | CD2 | TYR A | 150 | 22.789 | 0.299 | 0.668 | 1.00 | 24.35 |
| 1230 | CE2 | TYR A | 150 | 21.412 | 0.374 | 0.909 | 1.00 | 20.64 |
| 1231 | CZ | TYR A | 150 | 20.784 | 1.599 | 0.933 | 1.00 | 30.87 |
| 1232 | OH | TYR A | 150 | 19.428 | 1.673 | 1.174 | 1.00 | 44.40 |
| 1233 | C | TYR A | 150 | 25.492 | 2.711 | 2.250 | 1.00 | 39.54 |
| 1234 | O | TYR A | 150 | 24.661 | 2.544 | 3.170 | 1.00 | 10.94 |
| 1235 | N | CYS A | 151 | 26.062 | 3.882 | 1.987 | 1.00 | 36.69 |
| 1236 | CA | CYS A | 151 | 25.705 | 5.052 | 2.757 | 1.00 | 45.12 |
| 1237 | C | CYS A | 151 | 25.007 | 6.072 | 1.908 | 1.00 | 51.21 |
| 1238 | O | CYS A | 151 | 25.146 | 6.112 | 0.686 | 1.00 | 60.75 |
| 1239 | CB | CYS A | 151 | 26.931 | 5.677 | 3.410 | 1.00 | 44.14 |
| 1240 | SG | CYS A | 151 | 28.208 | 6.239 | 2.230 | 1.00 | 79.82 |
| 1241 | N | THR A | 152 | 24.258 | 6.891 | 2.606 | 1.00 | 48.42 |
| 1242 | CA | THR A | 152 | 23.500 | 7.959 | 1.949 | 1.00 | 39.06 |
| 1243 | CB | THR A | 152 | 22.068 | 7.517 | 1.650 | 1.00 | 43.65 |
| 1244 | OG1 | THR A | 152 | 21.399 | 8.531 | 0.891 | 1.00 | 41.96 |
| 1245 | CG2 | THR A | 152 | 21.310 | 7.257 | 2.942 | 1.00 | 52.65 |
| 1246 | C | THR A | 152 | 23.555 | 9.204 | 2.825 | 1.00 | 38.12 |
| 1247 | O | THR A | 152 | 23.025 | 9.224 | 3.943 | 1.00 | 25.44 |
| 1248 | N | GLY A | 153 | 24.215 | 10.236 | 2.307 | 1.00 | 56.51 |
| 1249 | CA | GLY A | 153 | 24.377 | 11.477 | 3.048 | 1.00 | 57.72 |
| 1250 | C | GLY A | 153 | 24.335 | 12.680 | 2.112 | 1.00 | 54.16 |
| 1251 | O | GLY A | 153 | 24.362 | 12.540 | 0.903 | 1.00 | 50.60 |
| 1252 | N | LYS A | 154 | 24.309 | 13.842 | 2.706 | 1.00 | 38.84 |
| 1253 | CA | LYS A | 154 | 24.227 | 14.969 | 1.840 | 1.00 | 51.33 |
| 1254 | CB | LYS A | 154 | 23.115 | 15.878 | 2.356 | 1.00 | 63.30 |
| 1255 | CG | LYS A | 154 | 23.445 | 17.360 | 2.388 | 1.00 | 72.63 |
| 1256 | CD | LYS A | 154 | 22.595 | 18.054 | 3.439 | 1.00 | 102.12 |
| 1257 | CE | LYS A | 154 | 23.004 | 19.508 | 3.589 | 1.00 | 114.39 |
| 1258 | NZ | LYS A | 154 | 22.404 | 20.128 | 4.797 | 1.00 | 116.75 |
| 1259 | C | LYS A | 154 | 25.591 | 15.650 | 1.647 | 1.00 | 43.23 |
| 1260 | O | LYS A | 154 | 26.393 | 15.769 | 2.565 | 1.00 | 25.84 |
| 1261 | N | VAL A | 155 | 25.799 | 16.110 | 0.410 | 1.00 | 50.07 |
| 1262 | CA | VAL A | 155 | 27.053 | 16.754 | 0.017 | 1.00 | 48.54 |
| 1263 | CB | VAL A | 155 | 27.920 | 15.801 | −0.810 | 1.00 | 44.27 |
| 1264 | CG1 | VAL A | 155 | 29.142 | 16.517 | −1.363 | 1.00 | 33.53 |
| 1265 | CG2 | VAL A | 155 | 28.341 | 14.619 | 0.032 | 1.00 | 57.29 |
| 1266 | C | VAL A | 155 | 26.752 | 18.008 | −0.791 | 1.00 | 67.17 |
| 1267 | O | VAL A | 155 | 26.127 | 17.926 | −1.839 | 1.00 | 75.50 |
| 1268 | N | TRP A | 156 | 27.200 | 19.158 | −0.290 | 1.00 | 71.27 |
| 1269 | CA | TRP A | 156 | 26.950 | 20.433 | −0.941 | 1.00 | 74.25 |
| 1270 | CB | TRP A | 156 | 27.792 | 20.531 | −2.227 | 1.00 | 82.77 |
| 1271 | CG | TRP A | 156 | 27.663 | 21.880 | −2.856 | 1.00 | 95.08 |
| 1272 | CD2 | TRP A | 156 | 27.991 | 23.118 | −2.237 | 1.00 | 99.06 |
| 1273 | CE2 | TRP A | 156 | 27.707 | 24.146 | −3.172 | 1.00 | 109.06 |
| 1274 | CE3 | TRP A | 156 | 28.499 | 23.466 | −0.981 | 1.00 | 92.83 |
| 1275 | CD1 | TRP A | 156 | 27.202 | 22.187 | −4.115 | 1.00 | 103.95 |
| 1276 | NE1 | TRP A | 156 | 27.226 | 23.548 | −4.308 | 1.00 | 101.14 |
| 1277 | CZ2 | TRP A | 156 | 27.915 | 25.505 | −2.883 | 1.00 | 117.16 |
| 1278 | CZ3 | TRP A | 156 | 28.708 | 24.820 | −0.691 | 1.00 | 111.82 |
| 1279 | CH2 | TRP A | 156 | 28.415 | 25.823 | −1.644 | 1.00 | 117.54 |
| 1280 | C | TRP A | 156 | 25.451 | 20.622 | −1.252 | 1.00 | 77.62 |
| 1281 | O | TRP A | 156 | 25.074 | 20.840 | −2.391 | 1.00 | 86.35 |
| 1282 | N | GLN A | 157 | 24.612 | 20.526 | −0.216 | 1.00 | 65.28 |
| 1283 | CA | GLN A | 157 | 23.148 | 20.731 | −0.292 | 1.00 | 81.65 |
| 1284 | CB | GLN A | 157 | 22.904 | 22.189 | −0.665 | 1.00 | 97.92 |
| 1285 | CG | GLN A | 157 | 23.502 | 23.199 | 0.312 | 1.00 | 117.66 |
| 1286 | CD | GLN A | 157 | 23.208 | 24.631 | −0.086 | 1.00 | 136.93 |
| 1287 | OE1 | GLN A | 157 | 22.533 | 24.882 | −1.084 | 1.00 | 146.04 |
| 1288 | NE2 | GLN A | 157 | 23.616 | 25.725 | 0.549 | 1.00 | 141.98 |
| 1289 | C | GLN A | 157 | 22.274 | 19.790 | −1.165 | 1.00 | 79.89 |
| 1290 | O | GLN A | 157 | 21.156 | 20.148 | −1.533 | 1.00 | 84.84 |
| 1291 | N | LEU A | 158 | 22.779 | 18.632 | −1.485 | 1.00 | 73.68 |
| 1292 | CA | LEU A | 158 | 22.000 | 17.633 | −2.253 | 1.00 | 79.20 |
| 1293 | CB | LEU A | 158 | 22.364 | 17.659 | −3.749 | 1.00 | 79.50 |
| 1294 | CG | LEU A | 158 | 22.096 | 18.967 | −4.507 | 1.00 | 86.09 |
| 1295 | CD1 | LEU A | 158 | 22.474 | 18.815 | −5.972 | 1.00 | 76.62 |
| 1296 | CD2 | LEU A | 158 | 20.647 | 19.379 | −4.373 | 1.00 | 92.41 |
| 1297 | C | LEU A | 158 | 22.262 | 16.262 | −1.673 | 1.00 | 70.21 |
| 1298 | O | LEU A | 158 | 23.390 | 15.914 | −1.325 | 1.00 | 65.09 |
| 1299 | N | ASP A | 159 | 21.236 | 15.470 | −1.538 | 1.00 | 60.67 |
| 1300 | CA | ASP A | 159 | 21.404 | 14.148 | −0.999 | 1.00 | 55.55 |
| 1301 | CB | ASP A | 159 | 20.088 | 13.617 | −0.446 | 1.00 | 67.04 |
| 1302 | CG | ASP A | 159 | 19.493 | 14.534 | 0.596 | 1.00 | 100.18 |
| 1303 | OD1 | ASP A | 159 | 20.141 | 14.767 | 1.640 | 1.00 | 107.25 |
| 1304 | OD2 | ASP A | 159 | 18.375 | 15.034 | 0.368 | 1.00 | 120.77 |
| 1305 | C | ASP A | 159 | 21.937 | 13.199 | −2.057 | 1.00 | 59.79 |
| 1306 | O | ASP A | 159 | 21.662 | 13.379 | −3.258 | 1.00 | 71.48 |
| 1307 | N | TYR A | 160 | 22.692 | 12.198 | −1.619 | 1.00 | 46.99 |
| 1308 | CA | TYR A | 160 | 23.237 | 11.218 | −2.545 | 1.00 | 36.10 |
| 1309 | CB | TYR A | 160 | 24.620 | 11.639 | −3.026 | 1.00 | 27.20 |
| 1310 | CG | TYR A | 160 | 24.661 | 13.032 | −3.599 | 1.00 | 48.59 |
| 1311 | CD1 | TYR A | 160 | 24.780 | 14.143 | −2.770 | 1.00 | 67.84 |
| 1312 | CE1 | TYR A | 160 | 24.805 | 15.440 | −3.297 | 1.00 | 85.53 |
| 1313 | CD2 | TYR A | 160 | 24.570 | 13.246 | −4.970 | 1.00 | 46.32 |
| 1314 | CE2 | TYR A | 160 | 24.592 | 14.538 | −5.507 | 1.00 | 59.84 |
| 1315 | CZ | TYR A | 160 | 24.713 | 15.630 | −4.665 | 1.00 | 78.07 |
| 1316 | OH | TYR A | 160 | 24.740 | 16.912 | −5.178 | 1.00 | 80.23 |
| 1317 | C | TYR A | 160 | 23.314 | 9.852 | −1.881 | 1.00 | 41.50 |
| 1318 | 0 | TYR A | 160 | 23.085 | 9.729 | −0.675 | 1.00 | 33.36 |
| 1319 | N | GLU A | 161 | 23.623 | 8.830 | −2.675 | 1.00 | 46.21 |
| 1320 | CA | GLU A | 161 | 23.725 | 7.469 | −2.173 | 1.00 | 42.10 |
| 1321 | CB | GLU A | 161 | 22.425 | 6.733 | −2.462 | 1.00 | 40.63 |
| 1322 | CG | GLU A | 161 | 22.451 | 5.247 | −2.195 | 1.00 | 72.70 |
| 1323 | CD | GLU A | 161 | 21.042 | 4.640 | −2.132 | 1.00 | 87.13 |
| 1324 | OE1 | GLU A | 161 | 20.932 | 3.391 | −2.193 | 1.00 | 96.87 |
| 1325 | OE2 | GLU A | 161 | 20.049 | 5.405 | −2.006 | 1.00 | 69.68 |
| 1326 | C | GLU A | 161 | 24.898 | 6.793 | −2.864 | 1.00 | 48.06 |
| 1327 | O | GLU A | 161 | 25.040 | 6.878 | −4.080 | 1.00 | 53.65 |
| 1328 | N | SER A | 162 | 25.750 | 6.146 | −2.079 | 1.00 | 55.15 |
| 1329 | CA | SER A | 162 | 26.933 | 5.475 | −2.612 | 1.00 | 57.03 |
| 1330 | CB | SER A | 162 | 27.968 | 5.246 | −1.498 | 1.00 | 68.17 |
| 1331 | OG | SER A | 162 | 27.499 | 4.339 | −0.501 | 1.00 | 46.93 |
| 1332 | C | SER A | 162 | 26.572 | 4.138 | −3.230 | 1.00 | 58.42 |
| 1333 | O | SER A | 162 | 25.476 | 3.615 | −3.020 | 1.00 | 48.37 |
| 1334 | N | GLU A | 163 | 27.498 | 3.587 | −4.002 | 1.00 | 65.57 |
| 1335 | CA | GLU A | 163 | 27.268 | 2.290 | −4.619 | 1.00 | 70.23 |
| 1336 | CB | GLU A | 163 | 28.356 | 1.990 | −5.650 | 1.00 | 87.77 |
| 1337 | CG | GLU A | 163 | 28.293 | 2.834 | −6.909 | 1.00 | 103.75 |
| 1338 | CD | GLU A | 163 | 27.121 | 2.453 | −7.797 | 1.00 | 115.27 |
| 1339 | OE1 | GLU A | 163 | 27.060 | 1.281 | −8.229 | 1.00 | 111.84 |
| 1340 | OE2 | GLU A | 163 | 26.262 | 3.323 | −8.062 | 1.00 | 129.74 |
| 1341 | C | GLU A | 163 | 27.366 | 1.282 | −3.491 | 1.00 | 70.72 |
| 1342 | O | GLU A | 163 | 28.244 | 1.387 | −2.645 | 1.00 | 89.57 |
| 1343 | N | PRO A | 164 | 26.460 | 0.300 | −3.448 | 1.00 | 55.48 |
| 1344 | CD | PRO A | 164 | 25.401 | −0.057 | −4.397 | 1.00 | 62.15 |
| 1345 | CA | PRO A | 164 | 26.538 | −0.684 | −2.369 | 1.00 | 41.51 |
| 1346 | CB | PRO A | 164 | 25.363 | −1.606 | −2.663 | 1.00 | 42.71 |
| 1347 | CG | PRO A | 164 | 25.250 | −1.532 | −4.128 | 1.00 | 64.09 |
| 1348 | C | PRO A | 164 | 27.877 | −1.409 | −2.399 | 1.00 | 38.62 |
| 1349 | 0 | PRO A | 164 | 28.585 | −1.386 | −3.426 | 1.00 | 31.41 |
| 1350 | N | LEU A | 165 | 28.234 | −2.022 | −1.268 | 1.00 | 33.57 |
| 1351 | CA | LEU A | 165 | 29.498 | −2.753 | −1.151 | 1.00 | 25.38 |
| 1352 | CB | LEU A | 165 | 30.540 | −1.879 | −0.490 | 1.00 | 26.45 |
| 1353 | CG | LEU A | 165 | 31.924 | −2.483 | −0.322 | 1.00 | 25.16 |
| 1354 | CD1 | LEU A | 165 | 32.619 | −2.579 | −1.655 | 1.00 | 29.84 |
| 1355 | CD2 | LEU A | 165 | 32.736 | −1.587 | 0.602 | 1.00 | 42.13 |
| 1356 | C | LEU A | 165 | 29.278 | −3.983 | −0.306 | 1.00 | 26.10 |
| 1357 | O | LEU A | 165 | 28.794 | −3.876 | 0.815 | 1.00 | 33.23 |
| 1358 | N | ASN A | 166 | 29.618 | −5.152 | −0.838 | 1.00 | 40.53 |
| 1359 | CA | ASN A | 166 | 29.400 | −6.398 | −0.106 | 1.00 | 50.87 |
| 1360 | CB | ASN A | 166 | 29.257 | −7.595 | −1.049 | 1.00 | 69.69 |
| 1361 | CG | ASN A | 166 | 27.875 | −7.698 | −1.649 | 1.00 | 86.10 |
| 1362 | OD1 | ASN A | 166 | 26.895 | −7.260 | −1.034 | 1.00 | 58.55 |
| 1363 | ND2 | ASN A | 166 | 27.799 | −8.292 | −2.839 | 1.00 | 110.79 |
| 1364 | C | ASN A | 166 | 30.537 | −6.664 | 0.833 | 1.00 | 48.09 |
| 1365 | O | ASN A | 166 | 31.703 | −6.667 | 0.416 | 1.00 | 40.29 |
| 1366 | N | ILE A | 167 | 30.193 | −6.908 | 2.094 | 1.00 | 46.84 |
| 1367 | CA | ILE A | 167 | 31.191 | −7.165 | 3.119 | 1.00 | 53.30 |
| 1368 | CB | ILE A | 167 | 31.192 | −6.039 | 4.156 | 1.00 | 54.88 |
| 1369 | CG2 | ILE A | 167 | 31.949 | −6.458 | 5.383 | 1.00 | 47.53 |
| 1370 | CG1 | ILE A | 167 | 31.816 | −4.791 | 3.545 | 1.00 | 48.70 |
| 1371 | CD1 | ILE A | 167 | 31.781 | −3.603 | 4.447 | 1.00 | 54.75 |
| 1372 | C | ILE A | 167 | 30.945 | −8.492 | 3.815 | 1.00 | 59.98 |
| 1373 | O | ILE A | 167 | 29.862 | −8.731 | 4.388 | 1.00 | 39.94 |
| 1374 | N | THR A | 168 | 31.957 | −9.353 | 3.777 | 1.00 | 61.65 |
| 1375 | CA | THR A | 168 | 31.835 | −10.666 | 4.386 | 1.00 | 68.33 |
| 1376 | CB | THR A | 168 | 32.052 | −11.774 | 3.343 | 1.00 | 80.46 |
| 1377 | OG1 | THR A | 168 | 31.627 | −11.306 | 2.058 | 1.00 | 92.03 |
| 1378 | CG2 | THR A | 168 | 31.239 | −13.010 | 3.704 | 1.00 | 94.56 |
| 1379 | C | THR A | 168 | 32.829 | −10.891 | 5.515 | 1.00 | 60.61 |
| 1380 | 0 | THR A | 168 | 34.031 | −10.670 | 5.346 | 1.00 | 54.25 |
| 1381 | N | VAL A | 169 | 32.313 | −11.331 | 6.660 | 1.00 | 57.24 |
| 1382 | CA | VAL A | 169 | 33.143 | −11.638 | 7.820 | 1.00 | 64.14 |
| 1383 | CB | VAL A | 169 | 32.567 | −11.028 | 9.083 | 1.00 | 54.63 |
| 1384 | CG1 | VAL A | 169 | 33.436 | −11.381 | 10.261 | 1.00 | 75.25 |
| 1385 | CG2 | VAL A | 169 | 32.470 | −9.533 | 8.929 | 1.00 | 61.93 |
| 1386 | C | VAL A | 169 | 33.112 | −13.156 | 7.950 | 1.00 | 73.51 |
| 1387 | O | VAL A | 169 | 32.044 | −13.739 | 8.173 | 1.00 | 77.56 |
| 1388 | N | ILE A | 170 | 34.268 | −13.801 | 7.815 | 1.00 | 75.88 |
| 1389 | CA | ILE A | 170 | 34.312 | −15.259 | 7.873 | 1.00 | 80.16 |
| 1390 | CB | ILE A | 170 | 35.341 | −15.815 | 6.866 | 1.00 | 75.70 |
| 1391 | CG2 | ILE A | 170 | 34.778 | −15.807 | 5.465 | 1.00 | 70.82 |
| 1392 | CG1 | ILE A | 170 | 36.633 | −15.007 | 6.932 | 1.00 | 90.89 |
| 1393 | CD1 | ILE A | 170 | 37.719 | −15.518 | 6.004 | 1.00 | 107.17 |
| 1394 | C | ILE A | 170 | 34.568 | −15.879 | 9.246 | 1.00 | 87.00 |
| 1395 | O | ILE A | 170 | 35.373 | −15.415 | 10.042 | 1.00 | 96.41 |
| 1396 | N | LYS A | 171 | 33.843 | −16.956 | 9.475 | 1.00 | 84.91 |
| 1397 | CA | LYS A | 171 | 34.002 | −17.747 | 10.684 | 1.00 | 77.44 |
| 1398 | CB | LYS A | 171 | 32.777 | −18.646 | 10.912 | 1.00 | 69.57 |
| 1399 | CG | LYS A | 171 | 32.895 | −19.571 | 12.108 | 1.00 | 99.45 |
| 1400 | CD | LYS A | 171 | 32.836 | −18.812 | 13.422 | 1.00 | 104.18 |
| 1401 | CE | LYS A | 171 | 32.865 | −19.773 | 14.611 | 1.00 | 104.74 |
| 1402 | NZ | LYS A | 171 | 32.737 | −19.061 | 15.914 | 1.00 | 93.90 |
| 1403 | C | LYS A | 171 | 35.260 | −18.572 | 10.523 | 1.00 | 84.98 |
| 1404 | 0 | LYS A | 171 | 35.628 | −18.860 | 9.376 | 1.00 | 94.71 |
| 1405 | N | ALA A | 172 | 35.970 | −19.012 | 11.575 | 1.00 | 87.21 |
| 1406 | CA | ALA A | 172 | 37.223 | −19.690 | 11.278 | 1.00 | 95.69 |
| 1407 | CB | ALA A | 172 | 38.361 | −18.758 | 11.670 | 1.00 | 93.23 |
| 1408 | C | ALA A | 172 | 37.489 | −21.071 | 11.877 | 1.00 | 105.19 |
| 1409 | O | ALA A | 172 | 37.954 | −21.216 | 13.004 | 1.00 | 112.63 |
| 1410 | N | PRO A | 173 | 37.149 | −22.090 | 11.058 | 1.00 | 110.62 |
| 1411 | CD | PRO A | 173 | 35.763 | −22.015 | 10.625 | 1.00 | 99.89 |
| 1412 | CA | PRO A | 173 | 37.466 | −23.510 | 11.384 | 1.00 | 115.87 |
| 1413 | CB | PRO A | 173 | 36.690 | −24.249 | 10.308 | 1.00 | 108.18 |
| 1414 | CG | PRO A | 173 | 35.438 | −23.407 | 10.162 | 1.00 | 104.37 |
| 1415 | C | PRO A | 173 | 38.974 | −23.790 | 11.504 | 1.00 | 128.43 |
| 1416 | O | PRO A | 173 | 39.763 | −23.104 | 10.878 | 1.00 | 141.54 |
| 1417 | N | ARg A | 174 | 39.371 | −24.790 | 12.289 | 1.00 | 133.44 |
| 1418 | CA | ARG A | 174 | 40.784 | −25.147 | 12.416 | 1.00 | 142.76 |
| 1419 | CB | ARG A | 174 | 41.404 | −24.373 | 13.578 | 1.00 | 151.84 |
| 1420 | CG | ARG A | 174 | 40.646 | −24.535 | 14.896 | 1.00 | 162.79 |
| 1421 | CD | ARG A | 174 | 40.099 | −23.221 | 15.420 | 1.00 | 169.70 |
| 1422 | NE | ARG A | 174 | 38.922 | −23.410 | 16.282 | 1.00 | 176.81 |
| 1423 | CZ | ARG A | 174 | 38.907 | −23.284 | 17.609 | 1.00 | 177.27 |
| 1424 | NH1 | ARG A | 174 | 40.007 | −22.942 | 18.271 | 1.00 | 174.36 |
| 1425 | NH2 | ARG A | 174 | 37.770 | −23.499 | 18.277 | 1.00 | 178.48 |
| 1426 | C | ARG A | 174 | 40.973 | −26.657 | 12.620 | 1.00 | 148.93 |
| 1427 | O | ARG A | 174 | 40.343 | −27.476 | 11.946 | 1.00 | 150.68 |
| 1428 | C1 | NAG A | 221 | 48.150 | 13.699 | −5.031 | 1.00 | 63.44 |
| 1429 | C2 | NAG A | 221 | 47.709 | 15.109 | −4.571 | 1.00 | 53.07 |
| 1430 | N2 | NAG A | 221 | 46.282 | 15.294 | −4.715 | 1.00 | 54.52 |
| 1431 | C7 | NAG A | 221 | 45.470 | 14.819 | −3.771 | 1.00 | 67.27 |
| 1432 | O7 | NAG A | 221 | 45.884 | 14.210 | −2.774 | 1.00 | 51.25 |
| 1433 | C8 | NAG A | 221 | 43.972 | 15.033 | −3.951 | 1.00 | 58.18 |
| 1434 | C3 | NAG A | 221 | 48.484 | 16.177 | −5.342 | 1.00 | 64.80 |
| 1435 | O3 | NAG A | 221 | 48.035 | 17.468 | −4.966 | 1.00 | 76.76 |
| 1436 | C4 | NAG A | 221 | 49.919 | 15.918 | −4.908 | 1.00 | 84.55 |
| 1437 | O4 | NAG A | 221 | 50.874 | 16.976 | −5.150 | 1.00 | 121.48 |
| 1438 | C5 | NAG A | 221 | 50.354 | 14.610 | −5.540 | 1.00 | 75.06 |
| 1439 | O5 | NAG A | 221 | 49.589 | 13.531 | −4.944 | 1.00 | 65.34 |
| 1440 | C6 | NAG A | 221 | 51.837 | 14.319 | −5.314 | 1.00 | 72.88 |
| 1441 | O6 | NAG A | 221 | 52.240 | 13.109 | −5.940 | 1.00 | 79.15 |
| 1442 | C1 | NAG A | 222 | 50.797 | 17.958 | −6.068 | 1.00 | 145.53 |
| 1443 | C2 | NAG A | 222 | 50.822 | 19.497 | −5.910 | 1.00 | 155.10 |
| 1444 | N2 | NAG A | 222 | 49.525 | 20.056 | −6.240 | 1.00 | 159.26 |
| 1445 | C7 | NAG A | 222 | 48.710 | 20.465 | −5.270 | 1.00 | 165.01 |
| 1446 | O7 | NAG A | 222 | 48.999 | 20.393 | −4.072 | 1.00 | 169.69 |
| 1447 | C8 | NAG A | 222 | 47.367 | 21.040 | −5.688 | 1.00 | 162.00 |
| 1448 | C3 | NAG A | 222 | 51.905 | 20.174 | −6.746 | 1.00 | 153.11 |
| 1449 | 03 | NAG A | 222 | 51.976 | 21.552 | −6.412 | 1.00 | 158.38 |
| 1450 | C4 | NAG A | 222 | 53.236 | 19.510 | −6.450 | 1.00 | 161.49 |
| 1451 | 04 | NAG A | 222 | 54.266 | 20.119 | −7.216 | 1.00 | 161.28 |
| 1452 | C5 | NAG A | 222 | 53.108 | 18.028 | −6.799 | 1.00 | 162.76 |
| 1453 | 05 | NAG A | 222 | 52.123 | 17.403 | −5.946 | 1.00 | 157.35 |
| 1454 | C6 | NAG A | 222 | 54.409 | 17.273 | −6.600 | 1.00 | 163.69 |
| 1455 | 06 | NAG A | 222 | 54.197 | 15.869 | −6.619 | 1.00 | 157.92 |
| 1456 | C1 | NAG A | 242 | 43.365 | −3.262 | −14.810 | 1.00 | 13.23 |
| 1457 | C2 | NAG A | 242 | 43.041 | −2.260 | −15.917 | 1.00 | 5.53 |
| 1458 | N2 | NAG A | 242 | 44.141 | −1.343 | −16.143 | 1.00 | 9.70 |
| 1459 | C7 | NAG A | 242 | 45.252 | −1.749 | −16.754 | 1.00 | 29.25 |
| 1460 | 07 | NAG A | 242 | 45.439 | −2.917 | −17.133 | 1.00 | 32.16 |
| 1461 | C8 | NAG A | 242 | 46.337 | −0.695 | −16.957 | 1.00 | 12.96 |
| 1462 | C3 | NAG A | 242 | 41.793 | −1.489 | −15.507 | 1.00 | 4.59 |
| 1463 | 03 | NAG A | 242 | 41.435 | −0.559 | −16.510 | 1.00 | 15.24 |
| 1464 | C4 | NAG A | 242 | 40.615 | −2.416 | −15.249 | 1.00 | 11.27 |
| 1465 | O4 | NAG A | 242 | 39.565 | −1.641 | −14.619 | 1.00 | 13.74 |
| 1466 | C5 | NAG A | 242 | 41.027 | −3.581 | −14.306 | 1.00 | 16.30 |
| 1467 | O5 | NAG A | 242 | 42.281 | −4.195 | −14.704 | 1.00 | 7.58 |
| 1468 | C6 | NAG A | 242 | 40.007 | −4.698 | −14.278 | 1.00 | 28.55 |
| 1469 | 06 | NAG A | 242 | 39.736 | −5.116 | −12.948 | 1.00 | 38.66 |
| 1470 | C1 | NAG A | 243 | 38.610 | −1.048 | −15.431 | 1.00 | 38.51 |
| 1471 | C2 | NAG A | 243 | 37.449 | −0.610 | −14.570 | 1.00 | 39.03 |
| 1472 | N2 | NAG A | 243 | 36.919 | −1.742 | −13.845 | 1.00 | 47.25 |
| 1473 | C7 | NAG A | 243 | 36.991 | −1.735 | −12.517 | 1.00 | 62.38 |
| 1474 | O7 | NAG A | 243 | 37.502 | −0.806 | −11.885 | 1.00 | 63.20 |
| 1475 | C8 | NAG A | 243 | 36.416 | −2.926 | −11.769 | 1.00 | 77.74 |
| 1476 | C3 | NAG A | 243 | 36.389 | 0.062 | −15.450 | 1.00 | 52.71 |
| 1477 | 03 | NAG A | 243 | 35.272 | 0.474 | −14.669 | 1.00 | 54.98 |
| 1478 | C4 | NAG A | 243 | 37.029 | 1.275 | −16.133 | 1.00 | 40.14 |
| 1479 | O4 | NAG A | 243 | 36.079 | 1.889 | −17.033 | 1.00 | 74.71 |
| 1480 | C5 | NAG A | 243 | 38.284 | 0.828 | −16.909 | 1.00 | 29.29 |
| 1481 | O5 | NAG A | 243 | 39.196 | 0.104 | −16.046 | 1.00 | 36.06 |
| 1482 | C6 | NAG A | 243 | 39.063 | 2.012 | −17.448 | 1.00 | 35.70 |
| 1483 | 06 | NAG A | 243 | 40.407 | 1.653 | −17.751 | 1.00 | 34.26 |
| 1484 | C1 | MAN A | 244 | 35.717 | 3.208 | −16.771 | 1.00 | 75.57 |
| 1485 | C2 | MAN A | 244 | 35.709 | 4.009 | −18.090 | 1.00 | 81.10 |
| 1486 | O2 | MAN A | 244 | 34.884 | 3.345 | −19.043 | 1.00 | 66.91 |
| 1487 | C3 | MAN A | 244 | 35.181 | 5.431 | −17.820 | 1.00 | 79.87 |
| 1488 | O3 | MAN A | 244 | 35.162 | 6.216 | −19.009 | 1.00 | 58.10 |
| 1489 | C4 | MAN A | 244 | 33.782 | 5.322 | −17.211 | 1.00 | 73.02 |
| 1490 | O4 | MAN A | 244 | 33.238 | 6.611 | −16.994 | 1.00 | 79.27 |
| 1491 | C5 | MAN A | 244 | 33.918 | 4.560 | −15.890 | 1.00 | 70.92 |
| 1492 | O5 | MAN A | 244 | 34.401 | 3.215 | −16.159 | 1.00 | 80.43 |
| 1493 | C6 | MAN A | 244 | 32.626 | 4.463 | −15.080 | 1.00 | 62.83 |
| 1494 | 06 | MAN A | 244 | 31.720 | 3.523 | −15.638 | 1.00 | 93.61 |
| 1495 | C1 | NAG A | 274 | 56.076 | −21.009 | −1.119 | 1.00 | 118.55 |
| 1496 | C2 | NAG A | 274 | 57.346 | −21.243 | −0.277 | 1.00 | 122.52 |
| 1497 | N2 | NAG A | 274 | 58.518 | −20.916 | −1.059 | 1.00 | 104.98 |
| 1498 | C7 | NAG A | 274 | 59.434 | −20.096 | −0.559 | 1.00 | 87.55 |
| 1499 | 07 | NAG A | 274 | 59.341 | −19.596 | 0.555 | 1.00 | 87.40 |
| 1500 | C8 | NAG A | 274 | 60.642 | −19.783 | −1.417 | 1.00 | 88.80 |
| 1501 | C3 | NAG A | 274 | 57.521 | −22.673 | 0.320 | 1.00 | 134.44 |
| 1502 | O3 | NAG A | 274 | 58.252 | −22.494 | 1.568 | 1.00 | 158.45 |
| 1503 | C4 | NAG A | 274 | 56.155 | −23.387 | 0.619 | 1.00 | 135.00 |
| 1504 | O4 | NAG A | 274 | 56.251 | −24.825 | 0.491 | 1.00 | 133.45 |
| 1505 | C5 | NAG A | 274 | 55.035 | −22.969 | −0.345 | 1.00 | 136.04 |
| 1506 | O5 | NAG A | 274 | 54.951 | −21.543 | −0.442 | 1.00 | 130.02 |
| 1507 | C6 | NAG A | 274 | 53.668 | −23.483 | 0.076 | 1.00 | 139.67 |
| 1508 | O6 | NAG A | 274 | 52.628 | −22.635 | −0.387 | 1.00 | 144.24 |
| 1509 | C1 | FCA A | 275 | 59.339 | −23.324 | 1.916 | 1.00 | 166.18 |
| 1510 | C2 | FCA A | 275 | 59.905 | −22.923 | 3.289 | 1.00 | 176.23 |
| 1511 | C3 | FCA A | 275 | 60.762 | −21.695 | 3.251 | 1.00 | 178.46 |
| 1512 | C4 | FCA A | 275 | 61.991 | −21.833 | 2.283 | 1.00 | 172.76 |
| 1513 | C5 | FCA A | 275 | 61.389 | −22.191 | 0.896 | 1.00 | 176.96 |
| 1514 | C6 | FCA A | 275 | 62.389 | −22.620 | −0.196 | 1.00 | 180.24 |
| 1515 | 02 | FCA A | 275 | 58.772 | −22.721 | 4.211 | 1.00 | 187.28 |
| 1516 | O3 | FCA A | 275 | 61.269 | −21.376 | 4.553 | 1.00 | 178.44 |
| 1517 | O4 | FCA A | 275 | 62.910 | −22.849 | 2.754 | 1.00 | 169.66 |
| 1518 | O5 | FCA A | 275 | 60.459 | −23.282 | 0.968 | 1.00 | 169.81 |
| 1519 | C1 | NAG A | 276 | 57.235 | −25.594 | 1.103 | 1.00 | 98.00 |
| 1520 | C2 | NAG A | 276 | 56.691 | −27.020 | 1.226 | 1.00 | 98.05 |
| 1521 | N2 | NAG A | 276 | 55.572 | −27.066 | 2.154 | 1.00 | 97.91 |
| 1522 | C7 | NAG A | 276 | 54.356 | −27.358 | 1.694 | 1.00 | 98.05 |
| 1523 | O7 | NAG A | 276 | 54.123 | −27.596 | 0.498 | 1.00 | 98.12 |
| 1524 | C8 | NAG A | 276 | 53.223 | −27.391 | 2.716 | 1.00 | 97.73 |
| 1525 | C3 | NAG A | 276 | 57.782 | −28.020 | 1.658 | 1.00 | 98.00 |
| 1526 | O3 | NAG A | 276 | 57.292 | −29.346 | 1.498 | 1.00 | 98.03 |
| 1527 | C4 | NAG A | 276 | 59.057 | −27.855 | 0.815 | 1.00 | 97.83 |
| 1528 | O4 | NAG A | 276 | 60.118 | −28.595 | 1.409 | 1.00 | 98.08 |
| 1529 | C5 | NAG A | 276 | 59.467 | −26.388 | 0.717 | 1.00 | 97.92 |
| 1530 | O5 | NAG A | 276 | 58.367 | −25.586 | 0.218 | 1.00 | 98.07 |
| 1531 | C6 | NAG A | 276 | 60.628 | −26.186 | −0.248 | 1.00 | 97.94 |
| 1532 | O6 | NAG A | 276 | 60.565 | −24.929 | −0.904 | 1.00 | 97.99 |
| 1533 | C1 | NAG A | 340 | 39.040 | −8.595 | 19.969 | 1.00 | 100.93 |
| 1534 | C2 | NAG A | 340 | 39.952 | −9.673 | 19.363 | 1.00 | 110.08 |
| 1535 | N2 | NAG A | 340 | 39.319 | −10.976 | 19.455 | 1.00 | 124.78 |
| 1536 | C7 | NAG A | 340 | 39.582 | −11.912 | 18.542 | 1.00 | 135.15 |
| 1537 | 07 | NAG A | 340 | 40.340 | −11.731 | 17.581 | 1.00 | 143.28 |
| 1538 | C8 | NAG A | 340 | 38.887 | −13.255 | 18.716 | 1.00 | 133.94 |
| 1539 | C3 | NAG A | 340 | 41.289 | −9.672 | 20.154 | 1.00 | 111.41 |
| 1540 | O3 | NAG A | 340 | 42.244 | −10.545 | 19.553 | 1.00 | 102.38 |
| 1541 | C4 | NAG A | 340 | 41.892 | −8.253 | 20.277 | 1.00 | 118.52 |
| 1542 | 04 | NAG A | 340 | 42.980 | −8.268 | 21.194 | 1.00 | 133.80 |
| 1543 | C5 | NAG A | 340 | 40.818 | −7.251 | 20.750 | 1.00 | 112.80 |
| 1544 | 05 | NAG A | 340 | 39.674 | −7.325 | 19.890 | 1.00 | 95.92 |
| 1545 | C6 | NAG A | 340 | 41.276 | −5.810 | 20.749 | 1.00 | 115.39 |
| 1546 | 06 | NAG A | 340 | 40.167 | −4.937 | 20.919 | 1.00 | 113.34 |
| 1547 | C1 | NAG A | 366 | 26.559 | −8.481 | −3.518 | 1.00 | 137.03 |
| 1548 | C2 | NAG A | 366 | 25.744 | −9.771 | −3.450 | 1.00 | 148.09 |
| 1549 | N2 | NAG A | 366 | 26.028 | −10.464 | −2.209 | 1.00 | 155.02 |
| 1550 | C7 | NAG A | 366 | 25.085 | −10.564 | −1.276 | 1.00 | 162.56 |
| 1551 | 07 | NAG A | 366 | 23.951 | −10.097 | −1.415 | 1.00 | 164.09 |
| 1552 | C8 | NAG A | 366 | 25.455 | −11.299 | 0.005 | 1.00 | 163.28 |
| 1553 | C3 | NAG A | 366 | 26.084 | −10.660 | −4.651 | 1.00 | 155.95 |
| 1554 | 03 | NAG A | 366 | 25.247 | −11.807 | −4.658 | 1.00 | 159.81 |
| 1555 | C4 | NAG A | 366 | 25.893 | −9.878 | −5.955 | 1.00 | 160.49 |
| 1556 | 04 | NAG A | 366 | 26.355 | −10.659 | −7.048 | 1.00 | 169.57 |
| 1557 | C5 | NAG A | 366 | 26.666 | −8.550 | −5.904 | 1.00 | 155.12 |
| 1558 | O5 | NAG A | 366 | 26.272 | −7.789 | −4.739 | 1.00 | 140.93 |
| 1559 | C6 | NAG A | 366 | 26.413 | −7.679 | −7.123 | 1.00 | 152.76 |
| 1560 | 06 | NAG A | 366 | 26.023 | −6.364 | −6.753 | 1.00 | 149.51 |
| 1561 | CB | VAL B | 1 | 4.752 | 40.855 | 51.137 | 1.00 | 126.57 |
| 1562 | CG1 | VAL B | 1 | 5.003 | 40.880 | 49.633 | 1.00 | 131.97 |
| 1563 | CG2 | VAL B | 1 | 3.866 | 42.021 | 51.535 | 1.00 | 130.09 |
| 1564 | C | VAL B | 1 | 5.086 | 38.381 | 51.284 | 1.00 | 100.44 |
| 1565 | 0 | VAL B | 1 | 6.179 | 38.608 | 50.764 | 1.00 | 97.65 |
| 1566 | N | VAL B | 1 | 3.657 | 39.534 | 52.971 | 1.00 | 122.36 |
| 1567 | CA | VAL B | 1 | 4.091 | 39.511 | 51.550 | 1.00 | 110.89 |
| 1568 | N | PRO B | 2 | 4.743 | 37.150 | 51.681 | 1.00 | 91.57 |
| 1569 | CD | PRO B | 2 | 3.715 | 36.682 | 52.617 | 1.00 | 86.81 |
| 1570 | CA | PRO B | 2 | 5.701 | 36.083 | 51.401 | 1.00 | 92.03 |
| 1571 | CB | PRO B | 2 | 5.219 | 34.937 | 52.280 | 1.00 | 92.13 |
| 1572 | CG | PRO B | 2 | 4.469 | 35.618 | 53.356 | 1.00 | 98.41 |
| 1573 | C | PRO B | 2 | 5.482 | 35.783 | 49.928 | 1.00 | 104.42 |
| 1574 | O | PRO B | 2 | 4.381 | 35.956 | 49.405 | 1.00 | 108.80 |
| 1575 | N | GLN B | 3 | 6.522 | 35.337 | 49.251 | 1.00 | 115.31 |
| 1576 | CA | GLN B | 3 | 6.395 | 35.044 | 47.839 | 1.00 | 118.75 |
| 1577 | CB | GLN B | 3 | 7.319 | 35.945 | 47.050 | 1.00 | 132.23 |
| 1578 | CG | GLN B | 3 | 6.978 | 37.381 | 47.261 | 1.00 | 134.41 |
| 1579 | CD | GLN B | 3 | 5.850 | 37.837 | 46.374 | 1.00 | 126.94 |
| 1580 | OE1 | GLN B | 3 | 4.815 | 37.173 | 46.271 | 1.00 | 124.90 |
| 1581 | NE2 | GLN B | 3 | 6.038 | 38.982 | 45.724 | 1.00 | 122.46 |
| 1582 | C | GLN B | 3 | 6.793 | 33.624 | 47.639 | 1.00 | 113.83 |
| 1583 | 0 | GLN B | 3 | 7.983 | 33.320 | 47.488 | 1.00 | 116.50 |
| 1584 | N | LYS B | 4 | 5.812 | 32.736 | 47.641 | 1.00 | 104.77 |
| 1585 | CA | LYS B | 4 | 6.188 | 31.357 | 47.465 | 1.00 | 92.78 |
| 1586 | CB | LYS B | 4 | 4.972 | 30.448 | 47.319 | 1.00 | 100.70 |
| 1587 | CG | LYS B | 4 | 3.816 | 31.031 | 46.558 | 1.00 | 119.63 |
| 1588 | CD | LYS B | 4 | 2.912 | 29.900 | 46.094 | 1.00 | 129.60 |
| 1589 | CE | LYS B | 4 | 1.591 | 30.427 | 45.556 | 1.00 | 131.81 |
| 1590 | NZ | LYS B | 4 | 0.528 | 29.403 | 45.762 | 1.00 | 129.85 |
| 1591 | C | LYS B | 4 | 7.093 | 31.230 | 46.253 | 1.00 | 81.27 |
| 1592 | O | LYS B | 4 | 6.904 | 31.908 | 45.239 | 1.00 | 74.91 |
| 1593 | N | PRO B | 5 | 8.133 | 30.392 | 46.370 | 1.00 | 75.63 |
| 1594 | CD | PRO B | 5 | 8.529 | 29.619 | 47.557 | 1.00 | 71.19 |
| 1595 | CA | PRO B | 5 | 9.074 | 30.177 | 45.271 | 1.00 | 65.65 |
| 1596 | CB | PRO B | 5 | 10.020 | 29.110 | 45.820 | 1.00 | 54.74 |
| 1597 | CG | PRO B | 5 | 9.982 | 29.343 | 47.268 | 1.00 | 65.46 |
| 1598 | C | PRO B | 5 | 8.255 | 29.650 | 44.097 | 1.00 | 64.33 |
| 1599 | O | PRO B | 5 | 7.090 | 29.277 | 44.255 | 1.00 | 65.82 |
| 1600 | N | LYS B | 6 | 8.858 | 29.608 | 42.923 | 1.00 | 65.65 |
| 1601 | CA | LYS B | 6 | 8.152 | 29.109 | 41.762 | 1.00 | 62.04 |
| 1602 | CB | LYS B | 6 | 7.636 | 30.279 | 40.927 | 1.00 | 55.18 |
| 1603 | CG | LYS B | 6 | 6.697 | 29.866 | 39.817 | 1.00 | 89.38 |
| 1604 | CD | LYS B | 6 | 5.394 | 30.661 | 39.855 | 1.00 | 97.54 |
| 1605 | CE | LYS B | 6 | 4.431 | 30.196 | 38.756 | 1.00 | 92.63 |
| 1606 | NZ | LYS B | 6 | 3.107 | 30.872 | 38.837 | 1.00 | 79.21 |
| 1607 | C | LYS B | 6 | 9.121 | 28.262 | 40.961 | 1.00 | 65.56 |
| 1608 | O | LYS B | 6 | 10.042 | 28.789 | 40.325 | 1.00 | 67.90 |
| 1609 | N | VAL B | 7 | 8.931 | 26.946 | 41.015 | 1.00 | 70.60 |
| 1610 | CA | VAL B | 7 | 9.803 | 26.041 | 40.280 | 1.00 | 65.28 |
| 1611 | CB | VAL B | 7 | 9.507 | 24.560 | 40.595 | 1.00 | 57.35 |
| 1612 | CG1 | VAL B | 7 | 10.356 | 23.663 | 39.694 | 1.00 | 39.82 |
| 1613 | CG2 | VAL B | 7 | 9.806 | 24.266 | 42.064 | 1.00 | 63.76 |
| 1614 | C | VAL B | 7 | 9.621 | 26.268 | 38.785 | 1.00 | 60.19 |
| 1615 | 0 | VAL B | 7 | 8.498 | 26.325 | 38.280 | 1.00 | 56.68 |
| 1616 | N | SER B | 8 | 10.736 | 26.423 | 38.087 | 1.00 | 44.71 |
| 1617 | CA | SER B | 8 | 10.719 | 26.639 | 36.656 | 1.00 | 31.26 |
| 1618 | CB | SER B | 8 | 11.396 | 27.952 | 36.318 | 1.00 | 41.26 |
| 1619 | OG | SER B | 8 | 12.802 | 27.750 | 36.214 | 1.00 | 36.80 |
| 1620 | C | SER B | 8 | 11.540 | 25.504 | 36.055 | 1.00 | 48.76 |
| 1621 | 0 | SER B | 8 | 12.480 | 25.002 | 36.675 | 1.00 | 52.73 |
| 1622 | N | LEU B | 9 | 11.202 | 25.094 | 34.844 | 1.00 | 48.97 |
| 1623 | CA | LEU B | 9 | 11.947 | 24.009 | 34.225 | 1.00 | 39.06 |
| 1624 | CB | LEU B | 9 | 11.000 | 22.902 | 33.735 | 1.00 | 33.73 |
| 1625 | CG | LEU B | 9 | 9.752 | 22.521 | 34.533 | 1.00 | 40.03 |
| 1626 | CD1 | LEU B | 9 | 9.198 | 21.252 | 33.933 | 1.00 | 39.96 |
| 1627 | CD2 | LEU B | 9 | 10.071 | 22.302 | 35.993 | 1.00 | 58.00 |
| 1628 | C | LEU B | 9 | 12.764 | 24.506 | 33.041 | 1.00 | 33.09 |
| 1629 | O | LEU B | 9 | 12.353 | 25.413 | 32.317 | 1.00 | 31.06 |
| 1630 | N | ASN B | 10 | 13.930 | 23.908 | 32.848 | 1.00 | 19.95 |
| 1631 | CA | ASN B | 10 | 14.763 | 24.262 | 31.721 | 1.00 | 20.51 |
| 1632 | CB | ASN B | 10 | 15.833 | 25.276 | 32.096 | 1.00 | 41.33 |
| 1633 | CG | ASN B | 10 | 16.763 | 25.559 | 30.942 | 1.00 | 52.07 |
| 1634 | OD1 | ASN B | 10 | 16.325 | 26.031 | 29.890 | 1.00 | 83.63 |
| 1635 | ND2 | ASN B | 10 | 18.048 | 25.263 | 31.119 | 1.00 | 49.68 |
| 1636 | C | ASN B | 10 | 15.446 | 23.008 | 31.211 | 1.00 | 34.87 |
| 1637 | O | ASN B | 10 | 16.375 | 22.497 | 31.843 | 1.00 | 40.39 |
| 1638 | N | PRO B | 11 | 15.025 | 22.518 | 30.034 | 1.00 | 43.40 |
| 1639 | CD | PRO B | 11 | 15.817 | 21.532 | 29.277 | 1.00 | 30.44 |
| 1640 | CA | PRO B | 11 | 13.956 | 23.087 | 29.197 | 1.00 | 20.84 |
| 1641 | CB | PRO B | 11 | 14.085 | 22.307 | 27.918 | 1.00 | 36.80 |
| 1642 | CG | PRO B | 11 | 15.604 | 21.988 | 27.879 | 1.00 | 28.71 |
| 1643 | C | PRO B | 11 | 12.572 | 22.964 | 29.819 | 1.00 | 34.02 |
| 1644 | 0 | PRO B | 11 | 12.312 | 22.023 | 30.566 | 1.00 | 49.37 |
| 1645 | N | PRO B | 12 | 11.663 | 23.896 | 29.482 | 1.00 | 30.69 |
| 1646 | CD | PRO B | 12 | 11.938 | 24.837 | 28.384 | 1.00 | 30.68 |
| 1647 | CA | PRO B | 12 | 10.268 | 24.026 | 29.945 | 1.00 | 24.81 |
| 1648 | CB | PRO B | 12 | 9.668 | 25.062 | 28.995 | 1.00 | 34.63 |
| 1649 | CG | PRO B | 12 | 10.861 | 25.893 | 28.607 | 1.00 | 49.64 |
| 1650 | C | PRO B | 12 | 9.452 | 22.761 | 29.981 | 1.00 | 29.55 |
| 1651 | O | PRO B | 12 | 8.465 | 22.654 | 30.716 | 1.00 | 28.29 |
| 1652 | N | TRP B | 13 | 9.873 | 21.802 | 29.176 | 1.00 | 38.99 |
| 1653 | CA | TRP B | 13 | 9.188 | 20.527 | 29.057 | 1.00 | 44.73 |
| 1654 | CB | TRP B | 13 | 9.904 | 19.706 | 27.996 | 1.00 | 44.13 |
| 1655 | CG | TRP B | 13 | 10.282 | 20.578 | 26.874 | 1.00 | 32.01 |
| 1656 | CD2 | TRP B | 13 | 9.428 | 21.507 | 26.210 | 1.00 | 33.06 |
| 1657 | CE2 | TRP B | 13 | 10.213 | 22.177 | 25.247 | 1.00 | 37.35 |
| 1658 | CE3 | TRP B | 13 | 8.069 | 21.841 | 26.335 | 1.00 | 32.11 |
| 1659 | CD1 | TRP B | 13 | 11.520 | 20.712 | 26.306 | 1.00 | 30.68 |
| 1660 | NE1 | TRP B | 13 | 11.487 | 21.675 | 25.330 | 1.00 | 32.77 |
| 1661 | CZ2 | TRP B | 13 | 9.686 | 23.160 | 24.411 | 1.00 | 42.92 |
| 1662 | CZ3 | TRP B | 13 | 7.545 | 22.816 | 25.507 | 1.00 | 36.94 |
| 1663 | CH2 | TRP B | 13 | 8.354 | 23.466 | 24.553 | 1.00 | 51.15 |
| 1664 | C | TRP B | 13 | 9.079 | 19.747 | 30.356 | 1.00 | 42.74 |
| 1665 | O | TRP B | 13 | 10.070 | 19.236 | 30.866 | 1.00 | 27.87 |
| 1666 | N | ASN B | 14 | 7.862 | 19.660 | 30.879 | 1.00 | 39.20 |
| 1667 | CA | ASN B | 14 | 7.609 | 18.933 | 32.107 | 1.00 | 42.44 |
| 1668 | CB | ASN B | 14 | 6.354 | 19.469 | 32.774 | 1.00 | 50.46 |
| 1669 | CG | ASN B | 14 | 5.119 | 19.130 | 32.010 | 1.00 | 49.15 |
| 1670 | OD1 | ASN B | 14 | 4.967 | 19.509 | 30.852 | 1.00 | 79.01 |
| 1671 | ND2 | ASN B | 14 | 4.221 | 18.402 | 32.649 | 1.00 | 57.09 |
| 1672 | C | ASN B | 14 | 7.427 | 17.455 | 31.790 | 1.00 | 43.98 |
| 1673 | O | ASN B | 14 | 7.263 | 16.635 | 32.683 | 1.00 | 61.67 |
| 1674 | N | ARG B | 15 | 7.443 | 17.126 | 30.507 | 1.00 | 44.77 |
| 1675 | CA | ARG B | 15 | 7.293 | 15.749 | 30.065 | 1.00 | 37.62 |
| 1676 | CB | ARG B | 15 | 6.053 | 15.588 | 29.197 | 1.00 | 37.39 |
| 1677 | CG | ARG B | 15 | 4.972 | 16.603 | 29.455 | 1.00 | 44.80 |
| 1678 | CD | ARG B | 15 | 3.826 | 16.330 | 28.510 | 1.00 | 56.59 |
| 1679 | NE | ARG B | 15 | 3.226 | 15.032 | 28.781 | 1.00 | 41.31 |
| 1680 | CZ | ARG B | 15 | 2.570 | 14.311 | 27.883 | 1.00 | 40.12 |
| 1681 | NH1 | ARG B | 15 | 2.435 | 14.759 | 26.639 | 1.00 | 57.43 |
| 1682 | NH2 | ARG B | 15 | 2.033 | 13.155 | 28.246 | 1.00 | 28.82 |
| 1683 | C | ARG B | 15 | 8.522 | 15.500 | 29.221 | 1.00 | 31.33 |
| 1684 | O | ARG B | 15 | 8.737 | 16.159 | 28.202 | 1.00 | 34.58 |
| 1685 | N | ILE B | 16 | 9.328 | 14.535 | 29.621 | 1.00 | 29.80 |
| 1686 | CA | ILE B | 16 | 10.540 | 14.286 | 28.872 | 1.00 | 46.54 |
| 1687 | CB | ILE B | 16 | 11.728 | 14.912 | 29.604 | 1.00 | 56.61 |
| 1688 | CG2 | ILE B | 16 | 11.405 | 16.370 | 29.929 | 1.00 | 58.31 |
| 1689 | CG1 | ILE B | 16 | 11.988 | 14.150 | 30.912 | 1.00 | 61.86 |
| 1690 | CD1 | ILE B | 16 | 13.106 | 14.716 | 31.756 | 1.00 | 58.95 |
| 1691 | C | ILE B | 16 | 10.813 | 12.811 | 28.652 | 1.00 | 43.09 |
| 1692 | O | ILE B | 16 | 10.303 | 11.952 | 29.383 | 1.00 | 46.04 |
| 1693 | N | PHE B | 17 | 11.619 | 12.532 | 27.634 | 1.00 | 34.79 |
| 1694 | CA | PHE B | 17 | 12.001 | 11.167 | 27.303 | 1.00 | 46.60 |
| 1695 | CB | PHE B | 17 | 12.605 | 11.118 | 25.894 | 1.00 | 38.91 |
| 1696 | CG | PHE B | 17 | 11.585 | 11.032 | 24.793 | 1.00 | 49.23 |
| 1697 | CD1 | PHE B | 17 | 11.845 | 11.607 | 23.549 | 1.00 | 47.72 |
| 1698 | CD2 | PHE B | 17 | 10.387 | 10.354 | 24.982 | 1.00 | 39.73 |
| 1699 | CE1 | PHE B | 17 | 10.931 | 11.520 | 22.512 | 1.00 | 39.05 |
| 1700 | CE2 | PHE B | 17 | 9.465 | 10.258 | 23.957 | 1.00 | 33.44 |
| 1701 | CZ | PHE B | 17 | 9.736 | 10.840 | 22.713 | 1.00 | 50.08 |
| 1702 | C | PHE B | 17 | 13.028 | 10.639 | 28.305 | 1.00 | 53.02 |
| 1703 | O | PHE B | 17 | 13.828 | 11.405 | 28.859 | 1.00 | 46.68 |
| 1704 | N | LYS B | 18 | 12.996 | 9.330 | 28.536 | 1.00 | 49.36 |
| 1705 | CA | LYS B | 18 | 13.942 | 8.691 | 29.439 | 1.00 | 46.09 |
| 1706 | CB | LYS B | 18 | 13.694 | 7.183 | 29.470 | 1.00 | 30.09 |
| 1707 | CG | LYS B | 18 | 14.791 | 6.380 | 30.134 | 1.00 | 62.01 |
| 1708 | CD | LYS B | 18 | 14.541 | 4.890 | 29.967 | 1.00 | 69.67 |
| 1709 | CE | LYS B | 18 | 15.591 | 4.051 | 30.696 | 1.00 | 79.54 |
| 1710 | NZ | LYS B | 18 | 16.966 | 4.233 | 30.147 | 1.00 | 74.39 |
| 1711 | C | LYS B | 18 | 15.363 | 8.964 | 28.946 | 1.00 | 45.81 |
| 1712 | O | LYS B | 18 | 15.641 | 8.877 | 27.761 | 1.00 | 48.24 |
| 1713 | N | GLY B | 19 | 16.261 | 9.305 | 29.860 | 1.00 | 54.24 |
| 1714 | CA | GLY B | 19 | 17.634 | 9.564 | 29.470 | 1.00 | 48.03 |
| 1715 | C | GLY B | 19 | 17.942 | 11.011 | 29.142 | 1.00 | 57.65 |
| 1716 | O | GLY B | 19 | 19.110 | 11.364 | 28.964 | 1.00 | 57.96 |
| 1717 | N | GLU B | 20 | 16.916 | 11.855 | 29.050 | 1.00 | 59.66 |
| 1718 | CA | GLU B | 20 | 17.142 | 13.269 | 28.754 | 1.00 | 58.50 |
| 1719 | CB | GLU B | 20 | 15.900 | 13.889 | 28.106 | 1.00 | 70.52 |
| 1720 | CG | GLU B | 20 | 15.444 | 13.180 | 26.834 | 1.00 | 82.41 |
| 1721 | CD | GLU B | 20 | 14.502 | 14.026 | 25.992 | 1.00 | 79.78 |
| 1722 | OE1 | GLU B | 20 | 13.490 | 14.527 | 26.550 | 1.00 | 67.98 |
| 1723 | OE2 | GLU B | 20 | 14.786 | 14.176 | 24.776 | 1.00 | 80.75 |
| 1724 | C | GLU B | 20 | 17.474 | 14.021 | 30.046 | 1.00 | 52.56 |
| 1725 | O | GLU B | 20 | 17.266 | 13.492 | 31.143 | 1.00 | 35.18 |
| 1726 | N | ASN B | 21 | 17.999 | 15.244 | 29.920 | 1.00 | 59.52 |
| 1727 | CA | ASN B | 21 | 18.344 | 16.072 | 31.085 | 1.00 | 47.35 |
| 1728 | CB | ASN B | 21 | 19.753 | 16.640 | 30.956 | 1.00 | 30.47 |
| 1729 | CG | ASN B | 21 | 20.784 | 15.572 | 30.658 | 1.00 | 55.82 |
| 1730 | OD1 | ASN B | 21 | 20.688 | 14.447 | 31.168 | 1.00 | 46.16 |
| 1731 | ND2 | ASN B | 21 | 21.772 | 15.924 | 29.835 | 1.00 | 67.36 |
| 1732 | C | ASN B | 21 | 17.383 | 17.239 | 31.261 | 1.00 | 40.95 |
| 1733 | O | ASN B | 21 | 16.866 | 17.784 | 30.292 | 1.00 | 70.03 |
| 1734 | N | VAL B | 22 | 17.152 | 17.619 | 32.507 | 1.00 | 25.27 |
| 1735 | CA | VAL B | 22 | 16.276 | 18.735 | 32.829 | 1.00 | 32.44 |
| 1736 | CB | VAL B | 22 | 14.824 | 18.247 | 33.003 | 1.00 | 20.08 |
| 1737 | CG1 | VAL B | 22 | 14.692 | 17.429 | 34.270 | 1.00 | 24.18 |
| 1738 | CG2 | VAL B | 22 | 13.882 | 19.426 | 33.061 | 1.00 | 36.02 |
| 1739 | C | VAL B | 22 | 16.775 | 19.385 | 34.139 | 1.00 | 43.21 |
| 1740 | O | VAL B | 22 | 17.327 | 18.710 | 35.003 | 1.00 | 49.24 |
| 1741 | N | THR B | 23 | 16.583 | 20.689 | 34.289 | 1.00 | 39.88 |
| 1742 | CA | THR B | 23 | 17.030 | 21.383 | 35.489 | 1.00 | 33.49 |
| 1743 | CB | THR B | 23 | 18.165 | 22.352 | 35.174 | 1.00 | 34.45 |
| 1744 | OG1 | THR B | 23 | 19.216 | 21.655 | 34.493 | 1.00 | 42.02 |
| 1745 | CG2 | THR B | 23 | 18.693 | 22.967 | 36.456 | 1.00 | 52.09 |
| 1746 | C | THR B | 23 | 15.931 | 22.190 | 36.167 | 1.00 | 43.19 |
| 1747 | o | THR B | 23 | 15.405 | 23.157 | 35.608 | 1.00 | 46.71 |
| 1748 | N | LEU B | 24 | 15.591 | 21.797 | 37.383 | 1.00 | 44.29 |
| 1749 | CA | LEU B | 24 | 14.581 | 22.521 | 38.131 | 1.00 | 54.45 |
| 1750 | CB | LEU B | 24 | 13.911 | 21.596 | 39.141 | 1.00 | 48.21 |
| 1751 | CG | LEU B | 24 | 13.237 | 20.376 | 38.519 | 1.00 | 51.83 |
| 1752 | CD1 | LEU B | 24 | 12.426 | 19.622 | 39.562 | 1.00 | 56.70 |
| 1753 | CD2 | LEU B | 24 | 12.329 | 20.835 | 37.415 | 1.00 | 69.70 |
| 1754 | C | LEU B | 24 | 15.255 | 23.676 | 38.858 | 1.00 | 60.50 |
| 1755 | O | LEU B | 24 | 16.299 | 23.494 | 39.482 | 1.00 | 84.20 |
| 1756 | N | THR B | 25 | 14.669 | 24.865 | 38.771 | 1.00 | 56.15 |
| 1757 | CA | THR B | 25 | 15.238 | 26.025 | 39.439 | 1.00 | 48.18 |
| 1758 | CB | THR B | 25 | 15.715 | 27.055 | 38.435 | 1.00 | 36.73 |
| 1759 | OG1 | THR B | 25 | 16.498 | 26.395 | 37.429 | 1.00 | 43.20 |
| 1760 | CG2 | THR B | 25 | 16.584 | 28.095 | 39.134 | 1.00 | 58.01 |
| 1761 | C | THR B | 25 | 14.228 | 26.659 | 40.374 | 1.00 | 55.18 |
| 1762 | O | THR B | 25 | 13.051 | 26.810 | 40.019 | 1.00 | 39.05 |
| 1763 | N | CYS B | 26 | 14.700 | 27.010 | 41.571 | 1.00 | 69.20 |
| 1764 | CA | CYS B | 26 | 13.866 | 27.603 | 42.608 | 1.00 | 77.69 |
| 1765 | C | CYS B | 26 | 14.115 | 29.091 | 42.752 | 1.00 | 84.84 |
| 1766 | O | CYS B | 26 | 15.186 | 29.501 | 43.193 | 1.00 | 95.13 |
| 1767 | CB | CYS B | 26 | 14.146 | 26.925 | 43.947 | 1.00 | 70.99 |
| 1768 | SG | CYS B | 26 | 12.878 | 27.161 | 45.240 | 1.00 | 87.46 |
| 1769 | N | ASM B | 27 | 13.127 | 29.898 | 42.378 | 1.00 | 94.31 |
| 1770 | CA | ASN B | 27 | 13.234 | 31.349 | 42.491 | 1.00 | 97.02 |
| 1771 | CB | ASN B | 27 | 14.182 | 31.917 | 41.409 | 1.00 | 104.76 |
| 1772 | CG | ASN B | 27 | 13.616 | 31.796 | 39.988 | 1.00 | 106.25 |
| 1773 | OD1 | ASN B | 27 | 12.599 | 31.141 | 39.748 | 1.00 | 109.42 |
| 1774 | ND2 | ASN B | 27 | 14.293 | 32.434 | 39.035 | 1.00 | 104.71 |
| 1775 | C | ASN B | 27 | 11.848 | 31.975 | 42.388 | 1.00 | 95.21 |
| 1776 | 0 | ASN B | 27 | 10.979 | 31.462 | 41.684 | 1.00 | 83.43 |
| 1777 | N | GLY B | 28 | 11.639 | 33.069 | 43.112 | 1.00 | 106.07 |
| 1778 | CA | GLY B | 28 | 10.354 | 33.753 | 43.101 | 1.00 | 127.70 |
| 1779 | C | GLY B | 28 | 10.461 | 34.992 | 43.966 | 1.00 | 138.06 |
| 1780 | O | GLY B | 28 | 10.631 | 34.879 | 45.182 | 1.00 | 143.08 |
| 1781 | N | ASN B | 29 | 10.341 | 36.173 | 43.362 | 1.00 | 142.87 |
| 1782 | CA | ASN B | 29 | 10.498 | 37.424 | 44.099 | 1.00 | 153.48 |
| 1783 | CB | ASN B | 29 | 9.350 | 37.669 | 45.082 | 1.00 | 152.59 |
| 1784 | CG | ASN B | 29 | 9.535 | 38.959 | 45.876 | 1.00 | 158.12 |
| 1785 | OD1 | ASN B | 29 | 10.508 | 39.679 | 45.675 | 1.00 | 156.21 |
| 1786 | ND2 | ASN B | 29 | 8.607 | 39.253 | 46.772 | 1.00 | 158.95 |
| 1787 | C | ASN B | 29 | 11.787 | 37.198 | 44.865 | 1.00 | 161.65 |
| 1788 | O | ASN B | 29 | 11.820 | 37.250 | 46.094 | 1.00 | 164.05 |
| 1789 | N | ASN B | 30 | 12.842 | 36.920 | 44.109 | 1.00 | 173.43 |
| 1790 | CA | ASN B | 30 | 14.136 | 36.629 | 44.690 | 1.00 | 183.92 |
| 1791 | CB | ASN B | 30 | 15.174 | 36.378 | 43.593 | 1.00 | 190.23 |
| 1792 | CG | ASN B | 30 | 16.451 | 35.752 | 44.136 | 1.00 | 197.43 |
| 1793 | OD1 | ASN B | 30 | 16.552 | 35.442 | 45.325 | 1.00 | 203.09 |
| 1794 | ND2 | ASN B | 30 | 17.431 | 35.555 | 43.262 | 1.00 | 200.85 |
| 1795 | C | ASN B | 30 | 14.653 | 37.685 | 45.639 | 1.00 | 186.26 |
| 1796 | O | ASN B | 30 | 14.262 | 38.855 | 45.593 | 1.00 | 185.38 |
| 1797 | N | PHE B | 31 | 15.547 | 37.235 | 46.501 | 1.00 | 188.59 |
| 1798 | CA | PHE B | 31 | 16.173 | 38.036 | 47.500 | 1.00 | 194.52 |
| 1799 | CB | PHE B | 31 | 15.485 | 37.780 | 48.881 | 1.00 | 203.03 |
| 1800 | CG | PHE B | 31 | 15.971 | 38.766 | 49.886 | 1.00 | 210.47 |
| 1801 | CD1 | PHE B | 31 | 15.408 | 40.027 | 49.940 | 1.00 | 216.06 |
| 1802 | CD2 | PHE B | 31 | 16.993 | 38.456 | 50.730 | 1.00 | 211.10 |
| 1803 | CE1 | PHE B | 31 | 15.861 | 40.961 | 50.804 | 1.00 | 222.00 |
| 1804 | CE2 | PHE B | 31 | 17.485 | 39.387 | 51.600 | 1.00 | 215.51 |
| 1805 | CZ | PHE B | 31 | 16.924 | 40.643 | 51.640 | 1.00 | 219.04 |
| 1806 | C | PHE B | 31 | 17.649 | 37.672 | 47.559 | 1.00 | 191.71 |
| 1807 | O | PHE B | 31 | 18.344 | 37.980 | 48.535 | 1.00 | 197.27 |
| 1808 | N | PHE B | 32 | 18.115 | 36.994 | 46.528 | 1.00 | 183.37 |
| 1809 | CA | PHE B | 32 | 19.516 | 36.543 | 46.471 | 1.00 | 176.18 |
| 1810 | CB | PHE B | 32 | 20.457 | 37.696 | 46.108 | 1.00 | 171.85 |
| 1811 | CG | PHE B | 32 | 20.693 | 38.668 | 47.204 | 1.00 | 170.32 |
| 1812 | CD1 | PHE B | 32 | 21.637 | 38.396 | 48.187 | 1.00 | 169.89 |
| 1813 | CD2 | PHE B | 32 | 19.977 | 39.859 | 47.276 | 1.00 | 168.03 |
| 1814 | CE1 | PHE B | 32 | 21.868 | 39.298 | 49.221 | 1.00 | 164.25 |
| 1815 | CE2 | PHE B | 32 | 20.201 | 40.765 | 48.308 | 1.00 | 160.78 |
| 1816 | CZ | PHE B | 32 | 21.145 | 40.483 | 49.282 | 1.00 | 159.57 |
| 1817 | C | PHE B | 32 | 19.870 | 35.945 | 47.824 | 1.00 | 175.68 |
| 1818 | O | PHE B | 32 | 21.029 | 35.946 | 48.235 | 1.00 | 173.43 |
| 1819 | N | GLU B | 33 | 18.872 | 35.409 | 48.474 | 1.00 | 177.15 |
| 1820 | CA | GLU B | 33 | 19.012 | 34.712 | 49.721 | 1.00 | 177.13 |
| 1821 | CB | GLU B | 33 | 17.859 | 35.035 | 50.674 | 1.00 | 179.86 |
| 1822 | CG | GLU B | 33 | 18.021 | 34.459 | 52.073 | 1.00 | 176.70 |
| 1823 | CD | GLU B | 33 | 19.050 | 35.206 | 52.898 | 1.00 | 173.43 |
| 1824 | OE1 | GLU B | 33 | 19.758 | 36.067 | 52.334 | 1.00 | 173.17 |
| 1825 | OE2 | GLU B | 33 | 19.151 | 34.929 | 54.111 | 1.00 | 172.06 |
| 1826 | C | GLU B | 33 | 18.966 | 33.237 | 49.329 | 1.00 | 175.21 |
| 1827 | 0 | GLU B | 33 | 18.622 | 32.361 | 50.122 | 1.00 | 174.80 |
| 1828 | N | VAL B | 34 | 19.311 | 32.998 | 48.071 | 1.00 | 172.32 |
| 1829 | CA | VAL B | 34 | 19.336 | 31.679 | 47.454 | 1.00 | 166.26 |
| 1830 | CB | VAL B | 34 | 20.040 | 31.769 | 46.083 | 1.00 | 167.66 |
| 1831 | CG1 | VAL B | 34 | 20.242 | 30.393 | 45.487 | 1.00 | 171.78 |
| 1832 | CG2 | VAL B | 34 | 19.234 | 32.658 | 45.147 | 1.00 | 170.77 |
| 1833 | C | VAL B | 34 | 19.997 | 30.573 | 48.274 | 1.00 | 160.12 |
| 1834 | O | VAL B | 34 | 19.643 | 29.405 | 48.137 | 1.00 | 161.37 |
| 1835 | N | SER B | 35 | 20.953 | 30.940 | 49.117 | 1.00 | 154.76 |
| 1836 | CA | SER B | 35 | 21.668 | 29.962 | 49.933 | 1.00 | 144.31 |
| 1837 | CB | SER B | 35 | 22.442 | 30.673 | 51.049 | 1.00 | 145.69 |
| 1838 | OG | SER B | 35 | 21.565 | 31.183 | 52.038 | 1.00 | 143.47 |
| 1839 | C | SER B | 35 | 20.772 | 28.897 | 50.555 | 1.00 | 136.95 |
| 1840 | O | SER B | 35 | 21.184 | 27.749 | 50.708 | 1.00 | 135.37 |
| 1841 | N | SER B | 36 | 19.547 | 29.269 | 50.912 | 1.00 | 130.67 |
| 1842 | CA | SER B | 36 | 18.623 | 28.328 | 51.542 | 1.00 | 127.00 |
| 1843 | CB | SER B | 36 | 17.944 | 28.990 | 52.748 | 1.00 | 133.26 |
| 1844 | OG | SER B | 36 | 17.047 | 30.017 | 52.345 | 1.00 | 123.80 |
| 1845 | C | SER B | 36 | 17.545 | 27.766 | 50.615 | 1.00 | 123.00 |
| 1846 | O | SER B | 36 | 16.620 | 28.481 | 50.222 | 1.00 | 128.61 |
| 1847 | N | THR B | 37 | 17.657 | 26.480 | 50.282 | 1.00 | 107.86 |
| 1848 | CA | THR B | 37 | 16.675 | 25.821 | 49.426 | 1.00 | 88.91 |
| 1849 | CB | THR B | 37 | 17.089 | 25.793 | 47.928 | 1.00 | 87.15 |
| 1850 | OG1 | THR B | 37 | 17.358 | 27.116 | 47.445 | 1.00 | 64.20 |
| 1851 | CG2 | THR B | 37 | 15.955 | 25.213 | 47.103 | 1.00 | 90.90 |
| 1852 | C | THR B | 37 | 16.469 | 24.376 | 49.854 | 1.00 | 86.86 |
| 1853 | O | THR B | 37 | 17.427 | 23.669 | 50.168 | 1.00 | 83.90 |
| 1854 | N | LYS B | 38 | 15.212 | 23.948 | 49.845 | 1.00 | 82.11 |
| 1855 | CA | LYS B | 38 | 14.835 | 22.596 | 50.224 | 1.00 | 83.86 |
| 1856 | CB | LYS B | 38 | 13.885 | 22.630 | 51.427 | 1.00 | 86.79 |
| 1857 | CG | LYS B | 38 | 14.442 | 22.010 | 52.699 | 1.00 | 100.07 |
| 1858 | CD | LYS B | 38 | 15.694 | 22.734 | 53.185 | 1.00 | 104.27 |
| 1859 | CE | LYS B | 38 | 16.039 | 22.305 | 54.602 | 1.00 | 97.90 |
| 1860 | NZ | LYS B | 38 | 14.882 | 22.578 | 55.510 | 1.00 | 91.89 |
| 1861 | C | LYS B | 38 | 14.115 | 21.953 | 49.051 | 1.00 | 82.06 |
| 1862 | O | LYS B | 38 | 13.147 | 22.520 | 48.545 | 1.00 | 72.96 |
| 1863 | N | TRP B | 39 | 14.579 | 20.784 | 48.613 | 1.00 | 82.25 |
| 1864 | CA | TRP B | 39 | 13.915 | 20.097 | 47.508 | 1.00 | 66.31 |
| 1865 | CB | TRP B | 39 | 14.922 | 19.675 | 46.449 | 1.00 | 58.26 |
| 1866 | CG | TRP B | 39 | 15.429 | 20.832 | 45.665 | 1.00 | 63.66 |
| 1867 | CD2 | TRP B | 39 | 14.789 | 21.453 | 44.542 | 1.00 | 65.27 |
| 1868 | CE2 | TRP B | 39 | 15.607 | 22.534 | 44.139 | 1.00 | 67.87 |
| 1869 | CE3 | TRP B | 39 | 13.605 | 21.201 | 43.841 | 1.00 | 51.05 |
| 1870 | CD1 | TRP B | 39 | 16.574 | 21.541 | 45.891 | 1.00 | 62.46 |
| 1871 | NE1 | TRP B | 39 | 16.690 | 22.565 | 44.977 | 1.00 | 57.52 |
| 1872 | CZ2 | TRP B | 39 | 15.277 | 23.364 | 43.061 | 1.00 | 59.34 |
| 1873 | CZ3 | TRP B | 39 | 13.280 | 22.023 | 42.775 | 1.00 | 59.55 |
| 1874 | CH2 | TRP B | 39 | 14.114 | 23.093 | 42.395 | 1.00 | 51.52 |
| 1875 | C | TRP B | 39 | 13.113 | 18.888 | 47.971 | 1.00 | 57.60 |
| 1876 | O | TRP B | 39 | 13.484 | 18.191 | 48.920 | 1.00 | 57.66 |
| 1877 | N | PHE B | 40 | 11.993 | 18.652 | 47.304 | 1.00 | 54.42 |
| 1878 | CA | PHE B | 40 | 11.149 | 17.534 | 47.671 | 1.00 | 62.19 |
| 1879 | CB | PHE B | 40 | 9.926 | 18.018 | 48.449 | 1.00 | 54.85 |
| 1880 | CG | PHE B | 40 | 10.262 | 18.678 | 49.745 | 1.00 | 59.54 |
| 1881 | CD1 | PHE B | 40 | 10.602 | 20.025 | 49.783 | 1.00 | 63.79 |
| 1882 | CD2 | PHE B | 40 | 10.310 | 17.934 | 50.920 | 1.00 | 65.98 |
| 1883 | CE1 | PHE B | 40 | 10.988 | 20.623 | 50.981 | 1.00 | 81.46 |
| 1884 | CE2 | PHE B | 40 | 10.696 | 18.518 | 52.122 | 1.00 | 72.69 |
| 1885 | CZ | PHE B | 40 | 11.039 | 19.863 | 52.154 | 1.00 | 78.47 |
| 1886 | C | PHE B | 40 | 10.688 | 16.728 | 46.476 | 1.00 | 68.64 |
| 1887 | O | PHE B | 40 | 9.920 | 17.217 | 45.650 | 1.00 | 78.19 |
| 1888 | N | HIS B | 41 | 11.157 | 15.487 | 46.396 | 1.00 | 72.46 |
| 1889 | CA | HIS B | 41 | 10.778 | 14.593 | 45.314 | 1.00 | 69.06 |
| 1890 | CB | HIS B | 41 | 11.988 | 13.804 | 44.833 | 1.00 | 61.71 |
| 1891 | CG | HIS B | 41 | 11.706 | 12.972 | 43.630 | 1.00 | 66.30 |
| 1892 | CD2 | HIS B | 41 | 12.491 | 12.132 | 42.921 | 1.00 | 62.70 |
| 1893 | ND1 | HIS B | 41 | 10.476 | 12.970 | 43.011 | 1.00 | 68.58 |
| 1894 | CE1 | HIS B | 41 | 10.517 | 12.161 | 41.967 | 1.00 | 82.82 |
| 1895 | NE2 | HIS B | 41 | 11.728 | 11.640 | 41.890 | 1.00 | 79.59 |
| 1896 | C | HIS B | 41 | 9.713 | 13.641 | 45.839 | 1.00 | 66.17 |
| 1897 | O | HIS B | 41 | 9.971 | 12.842 | 46.727 | 1.00 | 64.30 |
| 1898 | N | ASN B | 42 | 8.511 | 13.726 | 45.288 | 1.00 | 75.54 |
| 1899 | CA | ASN B | 42 | 7.423 | 12.877 | 45.751 | 1.00 | 79.71 |
| 1900 | CB | ASN B | 42 | 7.739 | 11.397 | 45.530 | 1.00 | 78.21 |
| 1901 | CG | ASN B | 42 | 7.203 | 10.871 | 44.210 | 1.00 | 78.51 |
| 1902 | OD1 | ASN B | 42 | 6.080 | 11.203 | 43.803 | 1.00 | 76.64 |
| 1903 | ND2 | ASN B | 42 | 8.001 | 10.027 | 43.560 | 1.00 | 77.12 |
| 1904 | C | ASN B | 42 | 7.183 | 13.113 | 47.236 | 1.00 | 79.13 |
| 1905 | O | ASN B | 42 | 6.754 | 12.203 | 47.958 | 1.00 | 64.42 |
| 1906 | N | GLY B | 43 | 7.478 | 14.330 | 47.692 | 1.00 | 81.21 |
| 1907 | CA | GLY B | 43 | 7.276 | 14.662 | 49.091 | 1.00 | 80.48 |
| 1908 | C | GLY B | 43 | 8.459 | 14.460 | 50.020 | 1.00 | 64.04 |
| 1909 | O | GLY B | 43 | 8.452 | 14.985 | 51.123 | 1.00 | 63.95 |
| 1910 | N | SER B | 44 | 9.472 | 13.719 | 49.573 | 1.00 | 59.76 |
| 1911 | CA | SER B | 44 | 10.655 | 13.449 | 50.387 | 1.00 | 64.76 |
| 1912 | CB | SER B | 44 | 11.191 | 12.034 | 50.114 | 1.00 | 70.69 |
| 1913 | OG | SER B | 44 | 10.248 | 11.045 | 50.502 | 1.00 | 89.71 |
| 1914 | C | SER B | 44 | 11.758 | 14.454 | 50.134 | 1.00 | 65.31 |
| 1915 | O | SER B | 44 | 12.038 | 14.802 | 48.993 | 1.00 | 76.25 |
| 1916 | N | LEU B | 45 | 12.386 | 14.914 | 51.209 | 1.00 | 65.35 |
| 1917 | CA | LEU B | 45 | 13.471 | 15.877 | 51.100 | 1.00 | 61.98 |
| 1918 | CB | LEU B | 45 | 13.917 | 16.337 | 52.496 | 1.00 | 65.46 |
| 1919 | CG | LEU B | 45 | 15.182 | 17.202 | 52.529 | 1.00 | 79.05 |
| 1920 | CD1 | LEU B | 45 | 14.971 | 18.471 | 51.691 | 1.00 | 77.35 |
| 1921 | CD2 | LEU B | 45 | 15.526 | 17.548 | 53.971 | 1.00 | 72.35 |
| 1922 | C | LEU B | 45 | 14.663 | 15.278 | 50.351 | 1.00 | 60.60 |
| 1923 | 0 | LEU B | 45 | 15.109 | 14.168 | 50.638 | 1.00 | 63.58 |
| 1924 | N | SER B | 46 | 15.167 | 16.015 | 49.374 | 1.00 | 61.57 |
| 1925 | CA | SER B | 46 | 16.315 | 15.564 | 48.608 | 1.00 | 73.03 |
| 1926 | CB | SER B | 46 | 16.247 | 16.120 | 47.186 | 1.00 | 77.22 |
| 1927 | OG | SER B | 46 | 17.386 | 15.737 | 46.432 | 1.00 | 90.60 |
| 1928 | C | SER B | 46 | 17.569 | 16.083 | 49.302 | 1.00 | 80.37 |
| 1929 | O | SER B | 46 | 17.499 | 16.999 | 50.129 | 1.00 | 76.01 |
| 1930 | N | GLU B | 47 | 18.710 | 15.493 | 48.969 | 1.00 | 84.85 |
| 1931 | CA | GLU B | 47 | 19.974 | 15.930 | 49.548 | 1.00 | 84.12 |
| 1932 | CB | GLU B | 47 | 21.027 | 14.827 | 49.429 | 1.00 | 87.82 |
| 1933 | CG | GLU B | 47 | 20.659 | 13.538 | 50.145 | 1.00 | 101.06 |
| 1934 | CD | GLU B | 47 | 20.468 | 13.733 | 51.637 | 1.00 | 111.75 |
| 1935 | OE1 | GLU B | 47 | 20.638 | 14.876 | 52.113 | 1.00 | 115.76 |
| 1936 | OE2 | GLU B | 47 | 20.148 | 12.744 | 52.329 | 1.00 | 122.59 |
| 1937 | C | GLU B | 47 | 20.468 | 17.212 | 48.886 | 1.00 | 82.42 |
| 1938 | O | GLU B | 47 | 21.259 | 17.951 | 49.455 | 1.00 | 91.17 |
| 1939 | N | GLU B | 48 | 19.971 | 17.458 | 47.675 | 1.00 | 78.36 |
| 1940 | CA | GLU B | 48 | 20.339 | 18.649 | 46.918 | 1.00 | 77.76 |
| 1941 | CB | GLU B | 48 | 19.624 | 18.652 | 45.569 | 1.00 | 83.13 |
| 1942 | CG | GLU B | 48 | 19.932 | 19.876 | 44.726 | 1.00 | 92.74 |
| 1943 | CD | GLU B | 48 | 21.376 | 19.914 | 44.265 | 1.00 | 101.93 |
| 1944 | OE1 | GLU B | 48 | 21.677 | 19.317 | 43.203 | 1.00 | 102.91 |
| 1945 | OE2 | GLU B | 48 | 22.204 | 20.531 | 44.975 | 1.00 | 108.55 |
| 1946 | C | GLU B | 48 | 19.970 | 19.916 | 47.686 | 1.00 | 77.31 |
| 1947 | 0 | GLU B | 48 | 18.853 | 20.040 | 48.204 | 1.00 | 74.82 |
| 1948 | N | THR B | 49 | 20.900 | 20.864 | 47.749 | 1.00 | 78.12 |
| 1949 | CA | THR B | 49 | 20.659 | 22.107 | 48.475 | 1.00 | 81.85 |
| 1950 | CB | THR B | 49 | 21.526 | 22.184 | 49.748 | 1.00 | 83.91 |
| 1951 | OG1 | THR B | 49 | 22.684 | 21.353 | 49.593 | 1.00 | 88.24 |
| 1952 | CG2 | THR B | 49 | 20.726 | 21.743 | 50.961 | 1.00 | 75.28 |
| 1953 | C | THR B | 49 | 20.871 | 23.390 | 47.680 | 1.00 | 82.91 |
| 1954 | 0 | THR B | 49 | 20.664 | 24.481 | 48.208 | 1.00 | 84.53 |
| 1955 | N | ASN B | 50 | 21.296 | 23.273 | 46.425 | 1.00 | 89.01 |
| 1956 | CA | ASN B | 50 | 21.491 | 24.455 | 45.592 | 1.00 | 90.45 |
| 1957 | CB | ASN B | 50 | 22.483 | 24.139 | 44.463 | 1.00 | 96.17 |
| 1958 | CG | ASN B | 50 | 22.910 | 25.375 | 43.697 | 1.00 | 111.81 |
| 1959 | OD1 | ASN B | 50 | 22.449 | 26.482 | 43.971 | 1.00 | 112.79 |
| 1960 | ND2 | ASN B | 50 | 23.798 | 25.190 | 42.727 | 1.00 | 110.81 |
| 1961 | C | ASN B | 50 | 20.123 | 24.856 | 45.013 | 1.00 | 89.21 |
| 1962 | O | ASN B | 50 | 19.208 | 24.033 | 44.942 | 1.00 | 94.63 |
| 1963 | N | SER B | 51 | 19.976 | 26.118 | 44.625 | 1.00 | 84.28 |
| 1964 | CA | SER B | 51 | 18.724 | 26.613 | 44.045 | 1.00 | 84.66 |
| 1965 | CB | SER B | 51 | 18.820 | 28.123 | 43.846 | 1.00 | 90.77 |
| 1966 | OG | SER B | 51 | 20.000 | 28.461 | 43.134 | 1.00 | 104.30 |
| 1967 | C | SER B | 51 | 18.387 | 25.940 | 42.699 | 1.00 | 77.58 |
| 1968 | O | 'SER B | 51 | 17.312 | 26.150 | 42.126 | 1.00 | 52.63 |
| 1969 | N | SER B | 52 | 19.313 | 25.133 | 42.195 | 1.00 | 74.45 |
| 1970 | CA | SER B | 52 | 19.108 | 24.441 | 40.934 | 1.00 | 62.55 |
| 1971 | CB | SER B | 52 | 20.120 | 24.930 | 39.907 | 1.00 | 56.77 |
| 1972 | OG | SER B | 52 | 20.003 | 26.329 | 39.742 | 1.00 | 76.18 |
| 1973 | C | SER B | 52 | 19.243 | 22.934 | 41.098 | 1.00 | 61.53 |
| 1974 | 0 | SER B | 52 | 20.327 | 22.425 | 41.391 | 1.00 | 66.28 |
| 1975 | N | LEU B | 53 | 18.133 | 22.229 | 40.915 | 1.00 | 56.00 |
| 1976 | CA | LEU B | 53 | 18.102 | 20.778 | 41.014 | 1.00 | 42.82 |
| 1977 | CB | LEU B | 53 | 16.789 | 20.323 | 41.642 | 1.00 | 35.21 |
| 1978 | CG | LEU B | 53 | 16.530 | 18.816 | 41.653 | 1.00 | 46.47 |
| 1979 | CD1 | LEU B | 53 | 17.775 | 18.057 | 42.111 | 1.00 | 51.40 |
| 1980 | CD2 | LEU B | 53 | 15.337 | 18.539 | 42.565 | 1.00 | 29.95 |
| 1981 | C | LEU B | 53 | 18.236 | 20.189 | 39.627 | 1.00 | 40.92 |
| 1982 | 0 | LEU B | 53 | 17.347 | 20.336 | 38.801 | 1.00 | 59.69 |
| 1983 | N | ASN B | 54 | 19.353 | 19.524 | 39.372 | 1.00 | 53.72 |
| 1984 | CA | ASN B | 54 | 19.593 | 18.921 | 38.068 | 1.00 | 63.37 |
| 1985 | CB | ASN B | 54 | 21.064 | 19.074 | 37.686 | 1.00 | 66.79 |
| 1986 | CG | ASN B | 54 | 21.475 | 20.516 | 37.562 | 1.00 | 81.16 |
| 1987 | OD1 | ASN B | 54 | 20.985 | 21.239 | 36.691 | 1.00 | 89.84 |
| 1988 | ND2 | ASN B | 54 | 22.372 | 20.952 | 38.439 | 1.00 | 99.57 |
| 1989 | C | ASN B | 54 | 19.205 | 17.444 | 37.989 | 1.00 | 68.27 |
| 1990 | O | ASN B | 54 | 19.398 | 16.681 | 38.935 | 1.00 | 72.52 |
| 1991 | N | ILE B | 55 | 18.643 | 17.061 | 36.849 | 1.00 | 66.50 |
| 1992 | CA | ILE B | 55 | 18.242 | 15.689 | 36.596 | 1.00 | 55.12 |
| 1993 | CB | ILE B | 55 | 16.744 | 15.574 | 36.310 | 1.00 | 34.50 |
| 1994 | CG2 | ILE B | 55 | 16.431 | 14.175 | 35.822 | 1.00 | 22.09 |
| 1995 | CG1 | ILE B | 55 | 15.952 | 15.909 | 37.577 | 1.00 | 32.78 |
| 1996 | CD1 | ILE B | 55 | 14.466 | 15.688 | 37.459 | 1.00 | 38.73 |
| 1997 | C | ILE B | 55 | 19.012 | 15.235 | 35.371 | 1.00 | 57.07 |
| 1998 | O | ILE B | 55 | 18.806 | 15.751 | 34.276 | 1.00 | 69.88 |
| 1999 | N | VAL B | 56 | 19.910 | 14.277 | 35.560 | 1.00 | 58.62 |
| 2000 | CA | VAL B | 56 | 20.719 | 13.795 | 34.450 | 1.00 | 65.98 |
| 2001 | CB | VAL B | 56 | 22.202 | 13.797 | 34.826 | 1.00 | 58.88 |
| 2002 | CG1 | VAL B | 56 | 23.047 | 13.490 | 33.609 | 1.00 | 54.44 |
| 2003 | CG2 | VAL B | 56 | 22.573 | 15.146 | 35.401 | 1.00 | 58.22 |
| 2004 | C | VAL B | 56 | 20.326 | 12.401 | 33.984 | 1.00 | 71.96 |
| 2005 | O | VAL B | 56 | 20.232 | 11.468 | 34.782 | 1.00 | 82.07 |
| 2006 | N | ASN B | 57 | 20.103 | 12.272 | 32.678 | 1.00 | 70.61 |
| 2007 | CA | ASN B | 57 | 19.704 | 11.006 | 32.081 | 1.00 | 79.87 |
| 2008 | CB | ASN B | 57 | 20.879 | 10.028 | 32.084 | 1.00 | 88.09 |
| 2009 | CG | ASN B | 57 | 22.082 | 10.573 | 31.331 | 1.00 | 104.06 |
| 2010 | OD1 | ASN B | 57 | 21.990 | 10.934 | 30.151 | 1.00 | 109.39 |
| 2011 | ND2 | ASN B | 57 | 23.220 | 10.640 | 32.013 | 1.00 | 110.76 |
| 2012 | C | ASN B | 57 | 18.532 | 10.441 | 32.865 | 1.00 | 77.25 |
| 2013 | O | ASN B | 57 | 18.597 | 9.335 | 33.403 | 1.00 | 83.22 |
| 2014 | N | ALA B | 58 | 17.463 | 11.229 | 32.916 | 1.00 | 69.58 |
| 2015 | CA | ALA B | 58 | 16.246 | 10.872 | 33.629 | 1.00 | 67.83 |
| 2016 | CB | ALA B | 58 | 15.101 | 11.748 | 33.155 | 1.00 | 70.80 |
| 2017 | C | ALA B | 58 | 15.854 | 9.399 | 33.519 | 1.00 | 66.71 |
| 2018 | 0 | ALA B | 58 | 15.912 | 8.790 | 32.455 | 1.00 | 63.76 |
| 2019 | N | LYS B | 59 | 15.472 | 8.837 | 34.655 | 1.00 | 62.18 |
| 2020 | CA | LYS B | 59 | 15.035 | 7.452 | 34.742 | 1.00 | 62.15 |
| 2021 | CB | LYS B | 59 | 15.688 | 6.774 | 35.943 | 1.00 | 77.31 |
| 2022 | CG | LYS B | 59 | 17.214 | 6.849 | 35.933 | 1.00 | 89.39 |
| 2023 | CD | LYS B | 59 | 17.816 | 6.471 | 37.277 | 1.00 | 86.69 |
| 2024 | CE | LYS B | 59 | 19.320 | 6.717 | 37.280 | 1.00 | 83.05 |
| 2025 | NZ | LYS B | 59 | 19.914 | 6.523 | 38.630 | 1.00 | 80.45 |
| 2026 | C | LYS B | 59 | 13.548 | 7.589 | 34.978 | 1.00 | 56.91 |
| 2027 | O | LYS B | 59 | 13.062 | 8.707 | 35.128 | 1.00 | 72.49 |
| 2028 | N | PHE B | 60 | 12.813 | 6.486 | 35.016 | 1.00 | 42.51 |
| 2029 | CA | PHE B | 60 | 11.380 | 6.608 | 35.244 | 1.00 | 48.10 |
| 2030 | CB | PHE B | 60 | 10.657 | 5.310 | 34.915 | 1.00 | 48.50 |
| 2031 | CG | PHE B | 60 | 10.808 | 4.884 | 33.480 | 1.00 | 64.06 |
| 2032 | CD1 | PHE B | 60 | 11.919 | 4.149 | 33.072 | 1.00 | 63.95 |
| 2033 | CD2 | PHE B | 60 | 9.837 | 5.216 | 32.533 | 1.00 | 74.15 |
| 2034 | CE1 | PHE B | 60 | 12.068 | 3.753 | 31.739 | 1.00 | 62.10 |
| 2035 | CE2 | PHE B | 60 | 9.977 | 4.826 | 31.196 | 1.00 | 74.92 |
| 2036 | CZ | PHE B | 60 | 11.092 | 4.089 | 30.801 | 1.00 | 69.78 |
| 2037 | C | PHE B | 60 | 11.166 | 6.962 | 36.693 | 1.00 | 55.02 |
| 2038 | O | PHE B | 60 | 10.203 | 7.639 | 37.058 | 1.00 | 61.86 |
| 2039 | N | GLU B | 61 | 12.097 | 6.503 | 37.517 | 1.00 | 75.45 |
| 2040 | CA | GLU B | 61 | 12.044 | 6.763 | 38.944 | 1.00 | 83.97 |
| 2041 | CB | GLU B | 61 | 13.190 | 6.038 | 39.668 | 1.00 | 96.33 |
| 2042 | CG | GLU B | 61 | 13.077 | 4.505 | 39.705 | 1.00 | 106.00 |
| 2043 | CD | GLU B | 61 | 13.368 | 3.847 | 38.362 | 1.00 | 110.30 |
| 2044 | OE1 | GLU B | 61 | 14.506 | 3.988 | 37.861 | 1.00 | 109.44 |
| 2045 | OE2 | GLU B | 61 | 12.459 | 3.187 | 37.811 | 1.00 | 111.21 |
| 2046 | C | GLU B | 61 | 12.136 | 8.267 | 39.188 | 1.00 | 78.63 |
| 2047 | O | GLU B | 61 | 11.814 | 8.747 | 40.277 | 1.00 | 79.41 |
| 2048 | N | ASP B | 62 | 12.579 | 9.007 | 38.174 | 1.00 | 64.96 |
| 2049 | CA | ASP B | 62 | 12.698 | 10.452 | 38.299 | 1.00 | 56.09 |
| 2050 | CB | ASP B | 62 | 13.720 | 11.010 | 37.306 | 1.00 | 58.67 |
| 2051 | CG | ASP B | 62 | 15.152 | 10.763 | 37.744 | 1.00 | 72.63 |
| 2052 | OD1 | ASP B | 62 | 15.467 | 11.068 | 38.916 | 1.00 | 85.98 |
| 2053 | OD2 | ASP B | 62 | 15.963 | 10.277 | 36.923 | 1.00 | 71.52 |
| 2054 | C | ASP B | 62 | 11.366 | 11.151 | 38.094 | 1.00 | 52.65 |
| 2055 | O | ASP B | 62 | 11.227 | 12.325 | 38.442 | 1.00 | 50.83 |
| 2056 | N | SER B | 63 | 10.391 | 10.440 | 37.533 | 1.00 | 47.35 |
| 2057 | CA | SER B | 63 | 9.076 | 11.029 | 37.311 | 1.00 | 51.69 |
| 2058 | CB | SER B | 63 | 8.157 | 10.038 | 36.609 | 1.00 | 59.14 |
| 2059 | OG | SER B | 63 | 8.676 | 9.676 | 35.341 | 1.00 | 76.77 |
| 2060 | C | SER B | 63 | 8.511 | 11.353 | 38.669 | 1.00 | 48.98 |
| 2061 | O | SER B | 63 | 9.048 | 10.900 | 39.666 | 1.00 | 55.63 |
| 2062 | N | GLY B | 64 | 7.439 | 12.135 | 38.717 | 1.00 | 50.75 |
| 2063 | CA | GLY B | 64 | 6.846 | 12.460 | 39.998 | 1.00 | 54.90 |
| 2064 | C | GLY B | 64 | 6.550 | 13.922 | 40.254 | 1.00 | 57.89 |
| 2065 | O | GLY B | 64 | 6.405 | 14.721 | 39.327 | 1.00 | 65.67 |
| 2066 | N | GLU B | 65 | 6.456 | 14.260 | 41.536 | 1.00 | 63.70 |
| 2067 | CA | GLU B | 65 | 6.161 | 15.619 | 41.982 | 1.00 | 62.19 |
| 2068 | CB | GLU B | 65 | 5.036 | 15.576 | 43.009 | 1.00 | 58.85 |
| 2069 | CG | GLU B | 65 | 4.715 | 16.899 | 43.646 | 1.00 | 74.39 |
| 2070 | CD | GLU B | 65 | 3.957 | 16.719 | 44.960 | 1.00 | 96.38 |
| 2071 | OE1 | GLU B | 65 | 4.578 | 16.285 | 45.968 | 1.00 | 90.14 |
| 2072 | OE2 | GLU B | 65 | 2.734 | 17.002 | 44.976 | 1.00 | 104.83 |
| 2073 | C | GLU B | 65 | 7.386 | 16.301 | 42.598 | 1.00 | 58.45 |
| 2074 | 0 | GLU B | 65 | 8.084 | 15.716 | 43.424 | 1.00 | 57.76 |
| 2075 | N | TYR B | 66 | 7.647 | 17.533 | 42.176 | 1.00 | 49.99 |
| 2076 | CA | TYR B | 66 | 8.768 | 18.291 | 42.696 | 1.00 | 34.15 |
| 2077 | CB | TYR B | 66 | 9.797 | 18.547 | 41.614 | 1.00 | 10.05 |
| 2078 | CG | TYR B | 66 | 10.595 | 17.338 | 41.213 | 1.00 | 33.40 |
| 2079 | CD1 | TYR B | 66 | 10.185 | 16.517 | 40.173 | 1.00 | 44.93 |
| 2080 | CE1 | TYR B | 66 | 10.957 | 15.427 | 39.766 | 1.00 | 55.60 |
| 2081 | CD2 | TYR B | 66 | 11.794 | 17.042 | 41.841 | 1.00 | 51.50 |
| 2082 | CE2 | TYR B | 66 | 12.574 | 15.951 | 41.445 | 1.00 | 59.78 |
| 2083 | CZ | TYR B | 66 | 12.154 | 15.150 | 40.405 | 1.00 | 62.67 |
| 2084 | OH | TYR B | 66 | 12.927 | 14.073 | 40.013 | 1.00 | 60.36 |
| 2085 | C | TYR B | 66 | 8.311 | 19.623 | 43.246 | 1.00 | 44.11 |
| 2086 | 0 | TYR B | 66 | 7.440 | 20.275 | 42.671 | 1.00 | 50.42 |
| 2087 | N | LYS B | 67 | 8.888 | 20.006 | 44.380 | 1.00 | 55.20 |
| 2088 | CA | LYS B | 67 | 8.577 | 21.275 | 45.043 | 1.00 | 54.38 |
| 2089 | CB | LYS B | 67 | 7.289 | 21.199 | 45.879 | 1.00 | 37.10 |
| 2090 | CG | LYS B | 67 | 7.088 | 19.925 | 46.675 | 1.00 | 40.24 |
| 2091 | CD | LYS B | 67 | 5.836 | 20.053 | 47.546 | 1.00 | 55.82 |
| 2092 | CE | LYS B | 67 | 5.362 | 18.702 | 48.076 | 1.00 | 71.24 |
| 2093 | NZ | LYS B | 67 | 4.355 | 18.855 | 49.172 | 1.00 | 79.55 |
| 2094 | C | LYS B | 67 | 9.725 | 21.684 | 45.931 | 1.00 | 61.59 |
| 2095 | 0 | LYS B | 67 | 10.222 | 20.891 | 46.730 | 1.00 | 80.11 |
| 2096 | N | CYS B | 68 | 10.161 | 22.924 | 45.761 | 1.00 | 69.06 |
| 2097 | CA | CYS B | 68 | 11.261 | 23.443 | 46.547 | 1.00 | 75.09 |
| 2098 | C | CYS B | 68 | 10.728 | 24.348 | 47.641 | 1.00 | 76.08 |
| 2099 | O | CYS B | 68 | 9.543 | 24.682 | 47.671 | 1.00 | 78.03 |
| 2100 | CB | CYS B | 68 | 12.231 | 24.217 | 45.665 | 1.00 | 71.35 |
| 2101 | SG | CYS B | 68 | 11.523 | 25.691 | 44.866 | 1.00 | 75.66 |
| 2102 | N | GLN B | 69 | 11.625 | 24.756 | 48.527 | 1.00 | 80.08 |
| 2103 | CA | GLN B | 69 | 11.233 | 25.600 | 49.635 | 1.00 | 74.62 |
| 2104 | CB | GLN B | 69 | 10.612 | 24.728 | 50.712 | 1.00 | 70.96 |
| 2105 | CG | GLN B | 69 | 10.067 | 25.488 | 51.859 | 1.00 | 72.60 |
| 2106 | CD | GLN B | 69 | 9.343 | 24.592 | 52.818 | 1.00 | 88.38 |
| 2107 | OE1 | GLN B | 69 | 9.406 | 23.363 | 52.720 | 1.00 | 85.19 |
| 2108 | NE2 | GLN B | 69 | 8.656 | 25.199 | 53.760 | 1.00 | 98.27 |
| 2109 | C | GLN B | 69 | 12.418 | 26.356 | 50.212 | 1.00 | 78.27 |
| 2110 | O | GLN B | 69 | 13.519 | 25.819 | 50.311 | 1.00 | 81.84 |
| 2111 | N | HIS B | 70 | 12.193 | 27.611 | 50.577 | 1.00 | 82.10 |
| 2112 | CA | HIS B | 70 | 13.252 | 28.398 | 51.183 | 1.00 | 85.15 |
| 2113 | CB | HIS B | 70 | 13.822 | 29.437 | 50.192 | 1.00 | 82.03 |
| 2114 | CG | HIS B | 70 | 12.892 | 30.565 | 49.860 | 1.00 | 93.26 |
| 2115 | CD2 | HIS B | 70 | 12.763 | 31.800 | 50.398 | 1.00 | 95.48 |
| 2116 | ND1 | HIS B | 70 | 11.997 | 30.515 | 48.812 | 1.00 | 109.66 |
| 2117 | CE1 | HIS B | 70 | 11.361 | 31.671 | 48.721 | 1.00 | 109.26 |
| 2118 | NE2 | HIS B | 70 | 11.804 | 32.468 | 49.672 | 1.00 | 109.02 |
| 2119 | C | HIS B | 70 | 12.735 | 29.044 | 52.469 | 1.00 | 85.57 |
| 2120 | 0 | HIS B | 70 | 11.549 | 29.350 | 52.598 | 1.00 | 85.74 |
| 2121 | N | GLN B | 71 | 13.635 | 29.213 | 53.431 | 1.00 | 79.77 |
| 2122 | CA | GLN B | 71 | 13.312 | 29.777 | 54.740 | 1.00 | 80.82 |
| 2123 | CB | GLN B | 71 | 14.619 | 30.126 | 55.465 | 1.00 | 89.59 |
| 2124 | CG | GLN B | 71 | 14.460 | 30.447 | 56.945 | 1.00 | 103.30 |
| 2125 | CD | GLN B | 71 | 15.762 | 30.921 | 57.585 | 1.00 | 109.00 |
| 2126 | OE1 | GLN B | 71 | 16.789 | 30.220 | 57.542 | 1.00 | 100.56 |
| 2127 | NE2 | GLN B | 71 | 15.726 | 32.120 | 58.184 | 1.00 | 102.47 |
| 2128 | C | GLN B | 71 | 12.365 | 30.993 | 54.763 | 1.00 | 67.00 |
| 2129 | O | GLN B | 71 | 12.486 | 31.913 | 53.953 | 1.00 | 42.78 |
| 2130 | N | GLN B | 72 | 11.432 | 30.978 | 55.715 | 1.00 | 69.70 |
| 2131 | CA | GLN B | 72 | 10.460 | 32.063 | 55.922 | 1.00 | 70.51 |
| 2132 | CB | GLN B | 72 | 11.188 | 33.388 | 56.149 | 1.00 | 78.43 |
| 2133 | CG | GLN B | 72 | 11.812 | 33.551 | 57.509 | 1.00 | 78.98 |
| 2134 | CD | GLN B | 72 | 12.598 | 34.838 | 57.593 | 1.00 | 95.13 |
| 2135 | OE1 | GLN B | 72 | 13.532 | 35.065 | 56.807 | 1.00 | 97.37 |
| 2136 | NE2 | GLN B | 72 | 12.223 | 35.700 | 58.540 | 1.00 | 98.58 |
| 2137 | C | GLN B | 72 | 9.398 | 32.282 | 54.840 | 1.00 | 66.32 |
| 2138 | 0 | GLN B | 72 | 8.737 | 33.329 | 54.816 | 1.00 | 61.96 |
| 2139 | N | VAL B | 73 | 9.227 | 31.302 | 53.960 | 1.00 | 63.38 |
| 2140 | CA | VAL B | 73 | 8.249 | 31.414 | 52.884 | 1.00 | 57.15 |
| 2141 | CB | VAL B | 73 | 8.933 | 31.810 | 51.592 | 1.00 | 31.26 |
| 2142 | CG1 | VAL B | 73 | 9.710 | 30.646 | 51.074 | 1.00 | 36.55 |
| 2143 | CG2 | VAL B | 73 | 7.918 | 32.256 | 50.585 | 1.00 | 50.56 |
| 2144 | C | VAL B | 73 | 7.552 | 30.075 | 52.663 | 1.00 | 59.51 |
| 2145 | O | VAL B | 73 | 8.148 | 29.022 | 52.896 | 1.00 | 77.00 |
| 2146 | N | ASN B | 74 | 6.300 | 30.115 | 52.210 | 1.00 | 61.46 |
| 2147 | CA | ASN B | 74 | 5.534 | 28.892 | 51.965 | 1.00 | 68.17 |
| 2148 | CB | ASN B | 74 | 4.195 | 29.223 | 51.312 | 1.00 | 73.30 |
| 2149 | CG | ASN B | 74 | 3.211 | 29.829 | 52.267 | 1.00 | 72.42 |
| 2150 | OD1 | ASN B | 74 | 2.787 | 29.182 | 53.225 | 1.00 | 62.22 |
| 2151 | ND2 | ASN B | 74 | 2.831 | 31.081 | 52.014 | 1.00 | 85.31 |
| 2152 | C | ASN B | 74 | 6.251 | 27.885 | 51.066 | 1.00 | 73.92 |
| 2153 | 0 | ASN B | 74 | 7.342 | 28.142 | 50.543 | 1.00 | 73.43 |
| 2154 | N | GLU B | 75 | 5.612 | 26.732 | 50.891 | 1.00 | 82.35 |
| 2155 | CA | GLU B | 75 | 6.138 | 25.682 | 50.029 | 1.00 | 88.01 |
| 2156 | CB | GLU B | 75 | 5.450 | 24.338 | 50.297 | 1.00 | 88.41 |
| 2157 | CG | GLU B | 75 | 5.962 | 23.548 | 51.490 | 1.00 | 102.21 |
| 2158 | CD | GLU B | 75 | 5.673 | 22.053 | 51.358 | 1.00 | 103.43 |
| 2159 | OE1 | GLU B | 75 | 4.497 | 21.692 | 51.118 | 1.00 | 95.26 |
| 2160 | OE2 | GLU B | 75 | 6.622 | 21.242 | 51.493 | 1.00 | 104.69 |
| 2161 | C | GLU B | 75 | 5.844 | 26.073 | 48.592 | 1.00 | 85.00 |
| 2162 | O | GLU B | 75 | 4.760 | 26.580 | 48.287 | 1.00 | 92.66 |
| 2163 | N | SER B | 76 | 6.799 | 25.830 | 47.705 | 1.00 | 74.73 |
| 2164 | CA | SER B | 76 | 6.597 | 26.136 | 46.299 | 1.00 | 64.93 |
| 2165 | CB | SER B | 76 | 7.811 | 25.716 | 45.474 | 1.00 | 65.57 |
| 2166 | OG | SER B | 76 | 7.884 | 24.300 | 45.366 | 1.00 | 53.84 |
| 2167 | C | SER B | 76 | 5.392 | 25.350 | 45.802 | 1.00 | 64.12 |
| 2168 | 0 | SER B | 76 | 4.920 | 24.425 | 46.467 | 1.00 | 55.19 |
| 2169 | N | GLU B | 77 | 4.891 | 25.745 | 44.639 | 1.00 | 73.18 |
| 2170 | CA | GLU B | 77 | 3.779 | 25.015 | 44.046 | 1.00 | 69.69 |
| 2171 | CB | GLU B | 77 | 3.077 | 25.874 | 42.993 | 1.00 | 59.63 |
| 2172 | CG | GLU B | 77 | 2.498 | 27.169 | 43.535 | 1.00 | 89.51 |
| 2173 | CD | GLU B | 77 | 1.820 | 28.000 | 42.462 | 1.00 | 107.00 |
| 2174 | OE1 | GLU B | 77 | 1.798 | 27.557 | 41.295 | 1.00 | 103.77 |
| 2175 | OE2 | GLU B | 77 | 1.312 | 29.093 | 42.789 | 1.00 | 116.96 |
| 2176 | C | GLU B | 77 | 4.246 | 23.698 | 43.437 | 1.00 | 63.68 |
| 2177 | O | GLU B | 77 | 5.367 | 23.648 | 42.922 | 1.00 | 69.58 |
| 2178 | N | PRO B | 78 | 3.458 | 22.659 | 43.542 | 1.00 | 56.73 |
| 2179 | CD | PRO B | 78 | 2.145 | 22.518 | 44.185 | 1.00 | 60.28 |
| 2180 | CA | PRO B | 78 | 3.910 | 21.387 | 42.987 | 1.00 | 49.00 |
| 2181 | CB | PRO B | 78 | 2.760 | 20.438 | 43.304 | 1.00 | 42.75 |
| 2182 | CG | PRO B | 78 | 2.141 | 21.046 | 44.534 | 1.00 | 68.49 |
| 2183 | C | PRO B | 78 | 4.155 | 21.509 | 41.497 | 1.00 | 44.78 |
| 2184 | O | PRO B | 78 | 3.483 | 22.260 | 40.795 | 1.00 | 52.51 |
| 2185 | N | VAL B | 79 | 5.137 | 20.766 | 41.024 | 1.00 | 34.40 |
| 2186 | CA | VAL B | 79 | 5.486 | 20.745 | 39.616 | 1.00 | 23.08 |
| 2187 | CB | VAL B | 79 | 6.779 | 21.527 | 39.364 | 1.00 | 17.91 |
| 2188 | CG1 | VAL B | 79 | 7.636 | 20.834 | 38.297 | 1.00 | 4.69 |
| 2189 | CG2 | VAL B | 79 | 6.413 | 22.948 | 38.961 | 1.00 | 17.83 |
| 2190 | C | VAL B | 79 | 5.674 | 19.278 | 39.257 | 1.00 | 40.76 |
| 2191 | O | VAL B | 79 | 6.573 | 18.609 | 39.783 | 1.00 | 37.75 |
| 2192 | N | TYR B | 80 | 4.831 | 18.773 | 38.362 | 1.00 | 43.38 |
| 2193 | CA | TYR B | 80 | 4.912 | 17.375 | 37.992 | 1.00 | 37.05 |
| 2194 | CB | TYR B | 80 | 3.510 | 16.807 | 37.858 | 1.00 | 19.64 |
| 2195 | CG | TYR B | 80 | 2.736 | 16.988 | 39.122 | 1.00 | 52.81 |
| 2196 | CD1 | TYR B | 80 | 1.881 | 18.084 | 39.293 | 1.00 | 64.76 |
| 2197 | CE1 | TYR B | 80 | 1.205 | 18.294 | 40.505 | 1.00 | 83.30 |
| 2198 | CD2 | TYR B | 80 | 2.901 | 16.098 | 40.186 | 1.00 | 64.99 |
| 2199 | CE2 | TYR B | 80 | 2.235 | 16.294 | 41.404 | 1.00 | 80.87 |
| 2200 | cz | TYR B | 80 | 1.390 | 17.394 | 41.559 | 1.00 | 84.40 |
| 2201 | OH | TYR B | 80 | 0.754 | 17.609 | 42.767 | 1.00 | 76.06 |
| 2202 | C | TYR B | 80 | 5.723 | 17.083 | 36.752 | 1.00 | 42.78 |
| 2203 | O | TYR B | 80 | 5.472 | 17.622 | 35.674 | 1.00 | 56.71 |
| 2204 | N | LEU B | 81 | 6.716 | 16.223 | 36.937 | 1.00 | 42.87 |
| 2205 | CA | LEU B | 81 | 7.615 | 15.798 | 35.878 | 1.00 | 47.81 |
| 2206 | CB | LEU B | 81 | 9.049 | 15.953 | 36.360 | 1.00 | 39.60 |
| 2207 | CG | LEU B | 81 | 10.151 | 15.383 | 35.481 | 1.00 | 53.84 |
| 2208 | CD1 | LEU B | 81 | 10.118 | 16.055 | 34.119 | 1.00 | 61.99 |
| 2209 | CD2 | LEU B | 81 | 11.494 | 15.611 | 36.160 | 1.00 | 65.74 |
| 2210 | C | LEU B | 81 | 7.312 | 14.328 | 35.562 | 1.00 | 60.00 |
| 2211 | O | LEU B | 81 | 7.172 | 13.508 | 36.467 | 1.00 | 72.31 |
| 2212 | N | GLU B | 82 | 7.208 | 13.992 | 34.282 | 1.00 | 60.42 |
| 2213 | CA | GLU B | 82 | 6.888 | 12.623 | 33.895 | 1.00 | 51.18 |
| 2214 | CB | GLU B | 82 | 5.425 | 12.559 | 33.475 | 1.00 | 60.43 |
| 2215 | CG | GLU B | 82 | 4.967 | 11.207 | 32.988 | 1.00 | 75.17 |
| 2216 | CD | GLU B | 82 | 3.453 | 11.151 | 32.824 | 1.00 | 92.83 |
| 2217 | OE1 | GLU B | 82 | 2.880 | 12.091 | 32.219 | 1.00 | 98.53 |
| 2218 | OE2 | GLU B | 82 | 2.839 | 10.166 | 33.301 | 1.00 | 97.49 |
| 2219 | C | GLU B | 82 | 7.778 | 12.089 | 32.780 | 1.00 | 49.31 |
| 2220 | O | GLU B | 82 | 7.734 | 12.564 | 31.645 | 1.00 | 54.46 |
| 2221 | N | VAL B | 83 | 8.590 | 11.095 | 33.116 | 1.00 | 44.15 |
| 2222 | CA | VAL B | 83 | 9.512 | 10.492 | 32.158 | 1.00 | 51.30 |
| 2223 | CB | VAL B | 83 | 10.656 | 9.749 | 32.862 | 1.00 | 44.14 |
| 2224 | CG1 | VAL B | 83 | 11.654 | 9.218 | 31.812 | 1.00 | 45.56 |
| 2225 | CG2 | VAL B | 83 | 11.334 | 10.669 | 33.861 | 1.00 | 46.11 |
| 2226 | C | VAL B | 83 | 8.820 | 9.488 | 31.246 | 1.00 | 55.74 |
| 2227 | 0 | VAL B | 83 | 8.110 | 8.609 | 31.713 | 1.00 | 73.69 |
| 2228 | N | PHE B | 84 | 9.051 | 9.608 | 29.945 | 1.00 | 53.23 |
| 2229 | CA | PHE B | 84 | 8.431 | 8.707 | 28.981 | 1.00 | 49.95 |
| 2230 | CB | PHE B | 84 | 7.631 | 9.481 | 27.929 | 1.00 | 49.58 |
| 2231 | CG | PHE B | 84 | 6.436 | 10.200 | 28.462 | 1.00 | 35.11 |
| 2232 | CD1 | PHE B | 84 | 6.570 | 11.175 | 29.423 | 1.00 | 52.71 |
| 2233 | CD2 | PHE B | 84 | 5.176 | 9.913 | 27.982 | 1.00 | 56.24 |
| 2234 | CE1 | PHE B | 84 | 5.463 | 11.859 | 29.898 | 1.00 | 63.67 |
| 2235 | CE2 | PHE B | 84 | 4.062 | 10.595 | 28.453 | 1.00 | 57.29 |
| 2236 | CZ | PHE B | 84 | 4.210 | 11.568 | 29.414 | 1.00 | 52.51 |
| 2237 | C | PHE B | 84 | 9.495 | 7.935 | 28.238 | 1.00 | 56.77 |
| 2238 | O | PHE B | 84 | 10.696 | 8.144 | 28.444 | 1.00 | 51.00 |
| 2239 | N | SER B | 85 | 9.021 | 7.056 | 27.360 | 1.00 | 59.31 |
| 2240 | CA | SER B | 85 | 9.856 | 6.236 | 26.496 | 1.00 | 53.43 |
| 2241 | CB | SER B | 85 | 10.382 | 5.014 | 27.230 | 1.00 | 52.20 |
| 2242 | OG | SER B | 85 | 11.313 | 4.342 | 26.403 | 1.00 | 68.06 |
| 2243 | C | SER B | 85 | 8.929 | 5.799 | 25.372 | 1.00 | 61.23 |
| 2244 | O | SER B | 85 | 8.107 | 4.897 | 25.547 | 1.00 | 56.17 |
| 2245 | N | ASP B | 86 | 9.053 | 6.474 | 24.234 | 1.00 | 68.29 |
| 2246 | CA | ASP B | 86 | 8.228 | 6.219 | 23.064 | 1.00 | 57.51 |
| 2247 | CB | ASP B | 86 | 6.812 | 6.736 | 23.321 | 1.00 | 51.72 |
| 2248 | CG | ASP B | 86 | 5.802 | 6.181 | 22.336 | 1.00 | 90.19 |
| 2249 | OD1 | ASP B | 86 | 6.002 | 6.371 | 21.113 | 1.00 | 100.01 |
| 2250 | OD2 | ASP B | 86 | 4.811 | 5.554 | 22.788 | 1.00 | 98.70 |
| 2251 | C | ASP B | 86 | 8.868 | 6.974 | 21.897 | 1.00 | 57.18 |
| 2252 | O | ASP B | 86 | 9.851 | 7.701 | 22.080 | 1.00 | 62.75 |
| 2253 | N | TRP B | 87 | 8.330 | 6.799 | 20.699 | 1.00 | 51.37 |
| 2254 | CA | TRP B | 87 | 8.896 | 7.483 | 19.543 | 1.00 | 50.71 |
| 2255 | CB | TRP B | 87 | 8.415 | 6.811 | 18.264 | 1.00 | 58.25 |
| 2256 | CG | TRP B | 87 | 8.811 | 5.379 | 18.134 | 1.00 | 53.11 |
| 2257 | CD2 | TRP B | 87 | 10.032 | 4.880 | 17.562 | 1.00 | 52.15 |
| 2258 | CE2 | TRP B | 87 | 9.945 | 3.476 | 17.552 | 1.00 | 60.06 |
| 2259 | CE3 | TRP B | 87 | 11.187 | 5.489 | 17.054 | 1.00 | 60.86 |
| 2260 | CD1 | TRP B | 87 | 8.060 | 4.285 | 18.452 | 1.00 | 44.63 |
| 2261 | NE1 | TRP B | 87 | 8.734 | 3.138 | 18.096 | 1.00 | 23.48 |
| 2262 | CZ2 | TRP B | 87 | 10.964 | 2.672 | 17.056 | 1.00 | 78.55 |
| 2263 | CZ3 | TRP B | 87 | 12.197 | 4.691 | 16.561 | 1.00 | 84.54 |
| 2264 | CH2 | TRP B | 87 | 12.077 | 3.294 | 16.565 | 1.00 | 87.37 |
| 2265 | C | TRP B | 87 | 8.545 | 8.982 | 19.495 | 1.00 | 42.37 |
| 2266 | O | TRP B | 87 | 9.367 | 9.839 | 19.123 | 1.00 | 23.61 |
| 2267 | N | LEU B | 88 | 7.308 | 9.296 | 19.849 | 1.00 | 29.09 |
| 2268 | CA | LEU B | 88 | 6.893 | 10.682 | 19.837 | 1.00 | 31.21 |
| 2269 | CB | LEU B | 88 | 5.817 | 10.944 | 18.777 | 1.00 | 33.13 |
| 2270 | CG | LEU B | 88 | 6.167 | 10.869 | 17.290 | 1.00 | 18.54 |
| 2271 | CD1 | LEU B | 88 | 5.021 | 11.506 | 16.542 | 1.00 | 25.10 |
| 2272 | CD2 | LEU B | 88 | 7.449 | 11.594 | 16.959 | 1.00 | 4.59 |
| 2273 | C | LEU B | 88 | 6.348 | 11.083 | 21.182 | 1.00 | 40.21 |
| 2274 | O | LEU B | 88 | 5.517 | 10.381 | 21.783 | 1.00 | 42.92 |
| 2275 | N | LEU B | 89 | 6.815 | 12.234 | 21.644 | 1.00 | 40.58 |
| 2276 | CA | LEU B | 89 | 6.369 | 12.780 | 22.912 | 1.00 | 31.34 |
| 2277 | CB | LEU B | 89 | 7.514 | 12.805 | 23.921 | 1.00 | 33.17 |
| 2278 | CG | LEU B | 89 | 7.202 | 13.378 | 25.299 | 1.00 | 42.18 |
| 2279 | CD1 | LEU B | 89 | 5.821 | 12.933 | 25.763 | 1.00 | 52.47 |
| 2280 | CD2 | LEU B | 89 | 8.282 | 12.923 | 26.264 | 1.00 | 49.06 |
| 2281 | C | LEU B | 89 | 5.899 | 14.194 | 22.621 | 1.00 | 27.75 |
| 2282 | 0 | LEU B | 89 | 6.617 | 14.973 | 21.947 | 1.00 | 5.35 |
| 2283 | N | LEU B | 90 | 4.703 | 14.517 | 23.109 | 1.00 | 4.73 |
| 2284 | CA | LEU B | 90 | 4.149 | 15.830 | 22.883 | 1.00 | 7.84 |
| 2285 | CB | LEU B | 90 | 2.634 | 15.775 | 22.724 | 1.00 | 21.81 |
| 2286 | CG | LEU B | 90 | 2.032 | 17.148 | 22.410 | 1.00 | 21.45 |
| 2287 | CD1 | LEU B | 90 | 2.511 | 17.587 | 21.026 | 1.00 | 29.29 |
| 2288 | CD2 | LEU B | 90 | 0.521 | 17.103 | 22.464 | 1.00 | 15.21 |
| 2289 | C | LEU B | 90 | 4.473 | 16.654 | 24.087 | 1.00 | 25.07 |
| 2290 | O | LEU B | 90 | 3.901 | 16.441 | 25.149 | 1.00 | 44.25 |
| 2291 | N | GLN B | 91 | 5.382 | 17.603 | 23.924 | 1.00 | 35.94 |
| 2292 | CA | GLN B | 91 | 5.768 | 18.461 | 25.028 | 1.00 | 35.91 |
| 2293 | CB | GLN B | 91 | 7.273 | 18.707 | 24.955 | 1.00 | 17.00 |
| 2294 | CG | GLN B | 91 | 8.061 | 17.428 | 25.129 | 1.00 | 13.88 |
| 2295 | CD | GLN B | 91 | 9.547 | 17.637 | 25.066 | 1.00 | 35.42 |
| 2296 | OE1 | GLN B | 91 | 10.092 | 18.074 | 24.040 | 1.00 | 18.59 |
| 2297 | NE2 | GLN B | 91 | 10.226 | 17.318 | 26.165 | 1.00 | 42.81 |
| 2298 | C | GLN B | 91 | 4.995 | 19.781 | 25.025 | 1.00 | 36.56 |
| 2299 | O | GLN B | 91 | 4.606 | 20.285 | 23.966 | 1.00 | 49.74 |
| 2300 | N | ALA B | 92 | 4.761 | 20.335 | 26.212 | 1.00 | 35.59 |
| 2301 | CA | ALA B | 92 | 4.054 | 21.607 | 26.313 | 1.00 | 43.59 |
| 2302 | CB | ALA B | 92 | 2.628 | 21.377 | 26.734 | 1.00 | 28.50 |
| 2303 | C | ALA B | 92 | 4.719 | 22.580 | 27.283 | 1.00 | 53.87 |
| 2304 | O | ALA B | 92 | 5.250 | 22.184 | 28.318 | 1.00 | 69.37 |
| 2305 | N | SER B | 93 | 4.684 | 23.860 | 26.932 | 1.00 | 68.52 |
| 2306 | CA | SER B | 93 | 5.252 | 24.923 | 27.761 | 1.00 | 64.18 |
| 2307 | CB | SER B | 93 | 4.947 | 26.287 | 27.133 | 1.00 | 72.74 |
| 2308 | OG | SER B | 93 | 3.537 | 26.475 | 26.986 | 1.00 | 77.53 |
| 2309 | C | SER B | 93 | 4.593 | 24.849 | 29.128 | 1.00 | 56.86 |
| 2310 | O | SER B | 93 | 5.200 | 25.163 | 30.143 | 1.00 | 67.19 |
| 2311 | N | ALA B | 94 | 3.336 | 24.430 | 29.131 | 1.00 | 36.84 |
| 2312 | CA | ALA B | 94 | 2.560 | 24.304 | 30.346 | 1.00 | 46.61 |
| 2313 | CB | ALA B | 94 | 2.296 | 25.671 | 30.923 | 1.00 | 39.28 |
| 2314 | C | ALA B | 94 | 1.248 | 23.611 | 29.999 | 1.00 | 57.58 |
| 2315 | O | ALA B | 94 | 0.553 | 24.008 | 29.063 | 1.00 | 64.68 |
| 2316 | N | GLU B | 95 | 0.912 | 22.571 | 30.754 | 1.00 | 65.01 |
| 2317 | CA | GLU B | 95 | −0.311 | 21.824 | 30.510 | 1.00 | 66.91 |
| 2318 | CB | GLU B | 95 | −0.290 | 20.535 | 31.329 | 1.00 | 69.50 |
| 2319 | CG | GLU B | 95 | 0.872 | 19.619 | 30.950 | 1.00 | 64.00 |
| 2320 | CD | GLU B | 95 | 0.886 | 18.326 | 31.738 | 1.00 | 82.38 |
| 2321 | OE1 | GLU B | 95 | 1.716 | 17.452 | 31.415 | 1.00 | 86.38 |
| 2322 | OE2 | GLU B | 95 | 0.075 | 18.181 | 32.678 | 1.00 | 93.94 |
| 2323 | C | GLU B | 95 | −1.540 | 22.669 | 30.831 | 1.00 | 68.95 |
| 2324 | O | GLU B | 95 | −2.644 | 22.394 | 30.346 | 1.00 | 67.01 |
| 2325 | N | VAL B | 96 | −1.345 | 23.703 | 31.644 | 1.00 | 72.58 |
| 2326 | CA | VAL B | 96 | −2.442 | 24.603 | 31.996 | 1.00 | 72.17 |
| 2327 | CB | VAL B | 96 | −3.012 | 24.282 | 33.374 | 1.00 | 56.87 |
| 2328 | CG1 | VAL B | 96 | −4.277 | 25.079 | 33.592 | 1.00 | 54.83 |
| 2329 | CG2 | VAL B | 96 | −3.280 | 22.788 | 33.489 | 1.00 | 38.14 |
| 2330 | C | VAL B | 96 | −1.980 | 26.059 | 31.988 | 1.00 | 76.73 |
| 2331 | O | VAL B | 96 | −1.079 | 26.441 | 32.738 | 1.00 | 76.80 |
| 2332 | N | VAL B | 97 | −2.611 | 26.867 | 31.141 | 1.00 | 80.52 |
| 2333 | CA | VAL B | 97 | −2.258 | 28.271 | 30.996 | 1.00 | 82.56 |
| 2334 | CB | VAL B | 97 | −1.740 | 28.546 | 29.562 | 1.00 | 85.79 |
| 2335 | CG1 | VAL B | 97 | −1.543 | 30.029 | 29.341 | 1.00 | 102.89 |
| 2336 | CG2 | VAL B | 97 | −0.429 | 27.803 | 29.334 | 1.00 | 95.31 |
| 2337 | C | VAL B | 97 | −3.420 | 29.216 | 31.283 | 1.00 | 82.63 |
| 2338 | 0 | VAL B | 97 | −4.591 | 28.885 | 31.041 | 1.00 | 75.81 |
| 2339 | N | MET B | 98 | −3.073 | 30.394 | 31.807 | 1.00 | 84.13 |
| 2340 | CA | MET B | 98 | −4.050 | 31.442 | 32.112 | 1.00 | 91.67 |
| 2341 | CB | MET B | 98 | −3.430 | 32.509 | 33.020 | 1.00 | 102.42 |
| 2342 | CG | MET B | 98 | −3.324 | 32.120 | 34.480 | 1.00 | 120.32 |
| 2343 | SD | MET B | 98 | −4.951 | 31.748 | 35.181 | 1.00 | 137.79 |
| 2344 | CE | MET B | 98 | −5.607 | 33.383 | 35.438 | 1.00 | 124.64 |
| 2345 | C | MET B | 98 | −4.488 | 32.107 | 30.808 | 1.00 | 86.05 |
| 2346 | 0 | MET B | 98 | −3.637 | 32.555 | 30.027 | 1.00 | 83.23 |
| 2347 | N | GLU B | 99 | −5.797 | 32.193 | 30.571 | 1.00 | 75.14 |
| 2348 | CA | GLU B | 99 | −6.266 | 32.803 | 29.334 | 1.00 | 80.10 |
| 2349 | CB | GLU B | 99 | −7.745 | 33.156 | 29.408 | 1.00 | 83.15 |
| 2350 | CG | GLU B | 99 | −8.228 | 33.862 | 28.144 | 1.00 | 109.03 |
| 2351 | CD | GLU B | 99 | −9.693 | 34.250 | 28.204 | 1.00 | 121.43 |
| 2352 | OE1 | GLU B | 99 | −10.084 | 34.917 | 29.189 | 1.00 | 131.12 |
| 2353 | OE2 | GLU B | 99 | −10.446 | 33.893 | 27.265 | 1.00 | 120.08 |
| 2354 | C | GLU B | 99 | −5.477 | 34.059 | 29.019 | 1.00 | 76.85 |
| 2355 | O | GLU B | 99 | −5.371 | 34.955 | 29.846 | 1.00 | 76.94 |
| 2356 | N | GLY B | 100 | −4.915 | 34.113 | 27.818 | 1.00 | 81.06 |
| 2357 | CA | GLY B | 100 | −4.139 | 35.273 | 27.429 | 1.00 | 80.78 |
| 2358 | C | GLY B | 100 | −2.644 | 35.015 | 27.460 | 1.00 | 77.00 |
| 2359 | O | GLY B | 100 | −1.877 | 35.674 | 26.752 | 1.00 | 79.67 |
| 2360 | N | GLN B | 101 | −2.220 | 34.062 | 28.284 | 1.00 | 75.05 |
| 2361 | CA | GLN B | 101 | −0.798 | 33.729 | 28.370 | 1.00 | 78.99 |
| 2362 | CB | GLN B | 101 | −0.494 | 32.996 | 29.682 | 1.00 | 84.76 |
| 2363 | CG | GLN B | 101 | −0.561 | 33.870 | 30.924 | 1.00 | 92.38 |
| 2364 | CD | GLN B | 101 | 0.340 | 35.097 | 30.817 | 1.00 | 98.22 |
| 2365 | OE1 | GLN B | 101 | −0.007 | 36.083 | 30.156 | 1.00 | 100.31 |
| 2366 | NE2 | GLN B | 101 | 1.509 | 35.036 | 31.456 | 1.00 | 92.08 |
| 2367 | C | GLN B | 101 | −0.351 | 32.876 | 27.178 | 1.00 | 65.25 |
| 2368 | 0 | GLN B | 101 | −1.169 | 32.375 | 26.414 | 1.00 | 57.39 |
| 2369 | N | PRO B | 102 | 0.963 | 32.708 | 26.997 | 1.00 | 59.88 |
| 2370 | CD | PRO B | 102 | 2.093 | 33.382 | 27.653 | 1.00 | 62.83 |
| 2371 | CA | PRO B | 102 | 1.422 | 31.901 | 25.868 | 1.00 | 59.94 |
| 2372 | CB | PRO B | 102 | 2.864 | 32.365 | 25.683 | 1.00 | 48.67 |
| 2373 | CG | PRO B | 102 | 3.284 | 32.645 | 27.070 | 1.00 | 63.93 |
| 2374 | C | PRO B | 102 | 1.313 | 30.411 | 26.120 | 1.00 | 60.51 |
| 2375 | O | PRO B | 102 | 1.559 | 29.938 | 27.220 | 1.00 | 71.07 |
| 2376 | N | LEU B | 103 | 0.926 | 29.686 | 25.081 | 1.00 | 66.31 |
| 2377 | CA | LEU B | 103 | 0.780 | 28.238 | 25.119 | 1.00 | 56.78 |
| 2378 | CB | LEU B | 103 | −0.664 | 27.880 | 24.821 | 1.00 | 50.78 |
| 2379 | CG | LEU B | 103 | −0.974 | 26.402 | 24.666 | 1.00 | 77.35 |
| 2380 | CD1 | LEU B | 103 | −0.629 | 25.680 | 25.955 | 1.00 | 76.88 |
| 2381 | CD2 | LEU B | 103 | −2.450 | 26.229 | 24.318 | 1.00 | 77.48 |
| 2382 | C | LEU B | 103 | 1.703 | 27.688 | 24.030 | 1.00 | 54.03 |
| 2383 | 0 | LEU B | 103 | 1.554 | 28.025 | 22.857 | 1.00 | 55.72 |
| 2384 | N | PHE B | 104 | 2.655 | 26.842 | 24.402 | 1.00 | 48.78 |
| 2385 | CA | PHE B | 104 | 3.607 | 26.332 | 23.414 | 1.00 | 45.32 |
| 2386 | CB | PHE B | 104 | 4.979 | 26.893 | 23.748 | 1.00 | 32.16 |
| 2387 | CG | PHE B | 104 | 5.976 | 26.705 | 22.677 | 1.00 | 32.32 |
| 2388 | CD1 | PHE B | 104 | 6.158 | 27.689 | 21.717 | 1.00 | 39.73 |
| 2389 | CD2 | PHE B | 104 | 6.770 | 25.566 | 22.641 | 1.00 | 39.96 |
| 2390 | CE1 | PHE B | 104 | 7.123 | 27.544 | 20.708 | 1.00 | 55.96 |
| 2391 | CE2 | PHE B | 104 | 7.744 | 25.404 | 21.638 | 1.00 | 63.91 |
| 2392 | CZ | PHE B | 104 | 7.923 | 26.404 | 20.673 | 1.00 | 61.86 |
| 2393 | C | PHE B | 104 | 3.700 | 24.805 | 23.299 | 1.00 | 42.46 |
| 2394 | O | PHE B | 104 | 4.206 | 24.140 | 24.199 | 1.00 | 46.85 |
| 2395 | N | LEU B | 105 | 3.223 | 24.250 | 22.190 | 1.00 | 35.08 |
| 2396 | CA | LEU B | 105 | 3.277 | 22.811 | 22.006 | 1.00 | 22.82 |
| 2397 | CB | LEU B | 105 | 1.999 | 22.311 | 21.356 | 1.00 | 37.29 |
| 2398 | CG | LEU B | 105 | 0.804 | 22.355 | 22.298 | 1.00 | 42.12 |
| 2399 | CD1 | LEU B | 105 | −0.404 | 21.635 | 21.660 | 1.00 | 56.32 |
| 2400 | CD2 | LEU B | 105 | 1.196 | 21.673 | 23.579 | 1.00 | 54.72 |
| 2401 | C | LEU B | 105 | 4.468 | 22.421 | 21.165 | 1.00 | 23.23 |
| 2402 | 0 | LEU B | 105 | 5.055 | 23.250 | 20.476 | 1.00 | 30.46 |
| 2403 | N | ARG B | 106 | 4.833 | 21.149 | 21.226 | 1.00 | 15.51 |
| 2404 | CA | ARG B | 106 | 5.990 | 20.674 | 20.475 | 1.00 | 30.48 |
| 2405 | CB | ARG B | 106 | 7.249 | 21.056 | 21.243 | 1.00 | 25.17 |
| 2406 | CG | ARG B | 106 | 8.540 | 20.446 | 20.746 | 1.00 | 50.57 |
| 2407 | CD | ARG B | 106 | 9.631 | 20.546 | 21.842 | 1.00 | 51.15 |
| 2408 | NE | ARG B | 106 | 10.970 | 20.223 | 21.346 | 1.00 | 50.88 |
| 2409 | CZ | ARG B | 106 | 12.013 | 19.938 | 22.120 | 1.00 | 42.48 |
| 2410 | NH1 | ARG B | 106 | 11.875 | 19.937 | 23.436 | 1.00 | 52.91 |
| 2411 | NH2 | ARG B | 106 | 13.191 | 19.650 | 21.581 | 1.00 | 40.82 |
| 2412 | C | ARG B | 106 | 5.913 | 19.143 | 20.289 | 1.00 | 37.36 |
| 2413 | O | ARG B | 106 | 5.488 | 18.406 | 21.199 | 1.00 | 26.46 |
| 2414 | N | CYS B | 107 | 6.304 | 18.660 | 19.113 | 1.00 | 28.15 |
| 2415 | CA | CYS B | 107 | 6.250 | 17.231 | 18.860 | 1.00 | 29.34 |
| 2416 | C | CYS B | 107 | 7.669 | 16.735 | 18.872 | 1.00 | 42.87 |
| 2417 | O | CYS B | 107 | 8.280 | 16.584 | 17.812 | 1.00 | 35.58 |
| 2418 | CB | CYS B | 107 | 5.617 | 16.945 | 17.502 | 1.00 | 38.81 |
| 2419 | SG | CYS B | 107 | 5.072 | 15.220 | 17.305 | 1.00 | 55.53 |
| 2420 | N | HIS B | 108 | 8.173 | 16.481 | 20.081 | 1.00 | 42.37 |
| 2421 | CA | HIS B | 108 | 9.544 | 16.022 | 20.296 | 1.00 | 43.07 |
| 2422 | CB | HIS B | 108 | 9.947 | 16.267 | 21.751 | 1.00 | 59.40 |
| 2423 | CG | HIS B | 108 | 11.374 | 15.931 | 22.052 | 1.00 | 55.40 |
| 2424 | CD2 | HIS B | 108 | 11.925 | 15.232 | 23.068 | 1.00 | 55.98 |
| 2425 | ND1 | HIS B | 108 | 12.421 | 16.349 | 21.259 | 1.00 | 54.99 |
| 2426 | CE1 | HIS B | 108 | 13.558 | 15.919 | 21.776 | 1.00 | 62.39 |
| 2427 | NE2 | HIS B | 108 | 13.286 | 15.239 | 22.874 | 1.00 | 62.88 |
| 2428 | C | HIS B | 108 | 9.739 | 14.563 | 19.946 | 1.00 | 36.76 |
| 2429 | 0 | HIS B | 108 | 9.008 | 13.698 | 20.440 | 1.00 | 16.46 |
| 2430 | N | GLY B | 109 | 10.733 | 14.307 | 19.097 | 1.00 | 27.59 |
| 2431 | CA | GLY B | 109 | 11.001 | 12.953 | 18.656 | 1.00 | 39.81 |
| 2432 | C | GLY B | 109 | 12.066 | 12.233 | 19.446 | 1.00 | 36.02 |
| 2433 | O | GLY B | 109 | 13.025 | 12.841 | 19.903 | 1.00 | 45.53 |
| 2434 | N | TRP B | 110 | 11.902 | 10.925 | 19.589 | 1.00 | 45.09 |
| 2435 | CA | TRP B | 110 | 12.842 | 10.094 | 20.328 | 1.00 | 42.50 |
| 2436 | CB | TRP B | 110 | 12.456 | 8.614 | 20.147 | 1.00 | 42.85 |
| 2437 | CG | TRP B | 110 | 13.388 | 7.739 | 20.893 | 1.00 | 47.55 |
| 2438 | CD2 | TRP B | 110 | 13.360 | 7.449 | 22.295 | 1.00 | 54.18 |
| 2439 | CE2 | TRP B | 110 | 14.455 | 6.601 | 22.586 | 1.00 | 43.50 |
| 2440 | CE3 | TRP B | 110 | 12.518 | 7.855 | 23.339 | 1.00 | 57.45 |
| 2441 | CD1 | TRP B | 110 | 14.459 | 7.056 | 20.399 | 1.00 | 51.38 |
| 2442 | NE1 | TRP B | 110 | 15.108 | 6.357 | 21.409 | 1.00 | 48.13 |
| 2443 | CZ2 | TRP B | 110 | 14.729 | 6.151 | 23.872 | 1.00 | 33.15 |
| 2444 | CZ3 | TRP B | 110 | 12.793 | 7.403 | 24.629 | 1.00 | 55.46 |
| 2445 | CH2 | TRP B | 110 | 13.894 | 6.557 | 24.877 | 1.00 | 49.35 |
| 2446 | C | TRP B | 110 | 14.276 | 10.354 | 19.886 | 1.00 | 45.65 |
| 2447 | O | TRP B | 110 | 14.544 | 10.511 | 18.690 | 1.00 | 34.73 |
| 2448 | N | ARG B | 111 | 15.182 | 10.388 | 20.866 | 1.00 | 55.00 |
| 2449 | CA | ARG B | 111 | 16.604 | 10.644 | 20.631 | 1.00 | 60.99 |
| 2450 | CB | ARG B | 111 | 17.254 | 9.438 | 19.949 | 1.00 | 56.42 |
| 2451 | CG | ARG B | 111 | 17.586 | 8.319 | 20.926 | 1.00 | 78.50 |
| 2452 | CD | ARG B | 111 | 18.140 | 7.100 | 20.224 | 1.00 | 104.44 |
| 2453 | NE | ARG B | 111 | 18.757 | 6.157 | 21.154 | 1.00 | 117.08 |
| 2454 | CZ | ARG B | 111 | 19.996 | 6.272 | 21.628 | 1.00 | 124.78 |
| 2455 | NH1 | ARG B | 111 | 20.766 | 7.291 | 21.261 | 1.00 | 125.05 |
| 2456 | NH2 | ARG B | 111 | 20.463 | 5.363 | 22.473 | 1.00 | 126.10 |
| 2457 | C | ARG B | 111 | 16.813 | 11.896 | 19.790 | 1.00 | 67.62 |
| 2458 | O | ARG B | 111 | 17.751 | 11.974 | 19.000 | 1.00 | 74.03 |
| 2459 | N | ASM B | 112 | 15.934 | 12.876 | 19.978 | 1.00 | 69.44 |
| 2460 | CA | ASN B | 112 | 16.039 | 14.116 | 19.228 | 1.00 | 76.51 |
| 2461 | CB | ASN B | 112 | 17.207 | 14.964 | 19.725 | 1.00 | 84.90 |
| 2462 | CG | ASN B | 112 | 17.150 | 16.373 | 19.160 | 1.00 | 106.45 |
| 2463 | OD1 | ASN B | 112 | 16.253 | 16.734 | 18.385 | 1.00 | 105.76 |
| 2464 | ND2 | ASN B | 112 | 18.134 | 17.183 | 19.546 | 1.00 | 117.72 |
| 2465 | C | ASN B | 112 | 16.155 | 13.785 | 17.751 | 1.00 | 79.76 |
| 2466 | O | ASN B | 112 | 16.695 | 14.568 | 16.973 | 1.00 | 88.06 |
| 2467 | N | TRP B | 113 | 15.686 | 12.614 | 17.372 | 1.00 | 76.72 |
| 2468 | CA | TRP B | 113 | 15.721 | 12.245 | 15.953 | 1.00 | 59.95 |
| 2469 | CB | TRP B | 113 | 15.222 | 10.811 | 15.765 | 1.00 | 54.79 |
| 2470 | CG | TRP B | 113 | 16.236 | 9.821 | 16.180 | 1.00 | 40.29 |
| 2471 | CD2 | TRP B | 113 | 16.046 | 8.442 | 16.523 | 1.00 | 18.18 |
| 2472 | CE2 | TRP B | 113 | 17.312 | 7.914 | 16.846 | 1.00 | 22.19 |
| 2473 | CE3 | TRP B | 113 | 14.930 | 7.603 | 16.587 | 1.00 | 12.77 |
| 2474 | CD1 | TRP B | 113 | 17.574 | 10.060 | 16.308 | 1.00 | 42.86 |
| 2475 | NE1 | TRP B | 113 | 18.226 | 8.922 | 16.707 | 1.00 | 25.08 |
| 2476 | CZ2 | TRP B | 113 | 17.493 | 6.587 | 17.228 | 1.00 | 22.94 |
| 2477 | CZ3 | TRP B | 113 | 15.111 | 6.286 | 16.966 | 1.00 | 28.48 |
| 2478 | CH2 | TRP B | 113 | 16.383 | 5.790 | 17.281 | 1.00 | 24.73 |
| 2479 | C | TRP B | 113 | 14.836 | 13.224 | 15.194 | 1.00 | 65.32 |
| 2480 | O | TRP B | 113 | 13.936 | 13.822 | 15.777 | 1.00 | 68.76 |
| 2481 | N | ASP B | 114 | 15.057 | 13.403 | 13.912 | 1.00 | 50.94 |
| 2482 | CA | ASP B | 114 | 14.164 | 14.252 | 13.145 | 1.00 | 52.09 |
| 2483 | CB | ASP B | 114 | 14.767 | 14.536 | 11.768 | 1.00 | 52.20 |
| 2484 | CG | ASP B | 114 | 16.039 | 15.346 | 11.830 | 1.00 | 66.27 |
| 2485 | OD1 | ASP B | 114 | 16.065 | 16.368 | 12.546 | 1.00 | 80.90 |
| 2486 | OD2 | ASP B | 114 | 17.011 | 14.967 | 11.147 | 1.00 | 65.81 |
| 2487 | C | ASP B | 114 | 12.798 | 13.615 | 13.004 | 1.00 | 54.32 |
| 2488 | O | ASP B | 114 | 12.676 | 12.397 | 13.005 | 1.00 | 49.44 |
| 2489 | N | VAL B | 115 | 11.762 | 14.429 | 12.886 | 1.00 | 59.65 |
| 2490 | CA | VAL B | 115 | 10.408 | 13.936 | 12.713 | 1.00 | 44.63 |
| 2491 | CB | VAL B | 115 | 9.542 | 14.291 | 13.899 | 1.00 | 43.22 |
| 2492 | CG1 | VAL B | 115 | 8.162 | 13.697 | 13.715 | 1.00 | 33.45 |
| 2493 | CG2 | VAL B | 115 | 10.192 | 13.802 | 15.156 | 1.00 | 33.33 |
| 2494 | C | VAL B | 115 | 9.876 | 14.672 | 11.497 | 1.00 | 60.55 |
| 2495 | O | VAL B | 115 | 10.254 | 15.827 | 11.261 | 1.00 | 71.83 |
| 2496 | N | TYR B | 116 | 9.023 | 14.021 | 10.715 | 1.00 | 48.40 |
| 2497 | CA | TYR B | 116 | 8.490 | 14.684 | 9.543 | 1.00 | 29.55 |
| 2498 | CB | TYR B | 116 | 9.214 | 14.204 | 8.299 | 1.00 | 25.24 |
| 2499 | CG | TYR B | 116 | 10.708 | 14.436 | 8.299 | 1.00 | 28.14 |
| 2500 | CD1 | TYR B | 116 | 11.582 | 13.475 | 8.783 | 1.00 | 40.15 |
| 2501 | CE1 | TYR B | 116 | 12.956 | 13.645 | 8.691 | 1.00 | 52.64 |
| 2502 | CD2 | TYR B | 116 | 11.250 | 15.590 | 7.745 | 1.00 | 41.11 |
| 2503 | CE2 | TYR B | 116 | 12.635 | 15.770 | 7.651 | 1.00 | 48.69 |
| 2504 | CZ | TYR B | 116 | 13.478 | 14.797 | 8.127 | 1.00 | 44.88 |
| 2505 | OH | TYR B | 116 | 14.841 | 14.957 | 8.017 | 1.00 | 54.67 |
| 2506 | C | TYR B | 116 | 6.991 | 14.501 | 9.377 | 1.00 | 27.78 |
| 2507 | 0 | TYR B | 116 | 6.383 | 13.681 | 10.051 | 1.00 | 28.35 |
| 2508 | N | LYS B | 117 | 6.409 | 15.265 | 8.460 | 1.00 | 38.88 |
| 2509 | CA | LYS B | 117 | 4.976 | 15.213 | 8.227 | 1.00 | 37.83 |
| 2510 | CB | LYS B | 117 | 4.567 | 13.922 | 7.508 | 1.00 | 47.24 |
| 2511 | CG | LYS B | 117 | 4.732 | 13.956 | 5.998 | 1.00 | 69.83 |
| 2512 | CD | LYS B | 117 | 4.053 | 12.743 | 5.350 | 1.00 | 93.57 |
| 2513 | CE | LYS B | 117 | 2.549 | 12.716 | 5.644 | 1.00 | 101.93 |
| 2514 | NZ | LYS B | 117 | 1.865 | 11.498 | 5.113 | 1.00 | 91.83 |
| 2515 | C | LYS B | 117 | 4.347 | 15.264 | 9.603 | 1.00 | 25.37 |
| 2516 | 0 | LYS B | 117 | 3.695 | 14.319 | 10.041 | 1.00 | 28.98 |
| 2517 | N | VAL B | 118 | 4.568 | 16.374 | 10.296 | 1.00 | 23.24 |
| 2518 | CA | VAL B | 118 | 4.014 | 16.513 | 11.629 | 1.00 | 23.69 |
| 2519 | CB | VAL B | 118 | 4.919 | 17.367 | 12.545 | 1.00 | 38.59 |
| 2520 | CG1 | VAL B | 118 | 4.205 | 17.627 | 13.868 | 1.00 | 45.19 |
| 2521 | CG2 | VAL B | 118 | 6.254 | 16.648 | 12.795 | 1.00 | 18.15 |
| 2522 | C | VAL B | 118 | 2.650 | 17.152 | 11.593 | 1.00 | 18.84 |
| 2523 | O | VAL B | 118 | 2.462 | 18.227 | 11.006 | 1.00 | 8.55 |
| 2524 | N | ILE B | 119 | 1.700 | 16.503 | 12.250 | 1.00 | 12.98 |
| 2525 | CA | ILE B | 119 | 0.355 | 17.026 | 12.293 | 1.00 | 30.22 |
| 2526 | CB | ILE B | 119 | −0.627 | 16.130 | 11.498 | 1.00 | 46.31 |
| 2527 | CG2 | ILE B | 119 | −2.064 | 16.621 | 11.688 | 1.00 | 42.76 |
| 2528 | CG1 | ILE B | 119 | −0.236 | 16.112 | 10.019 | 1.00 | 21.93 |
| 2529 | CD1 | ILE B | 119 | −1.236 | 15.436 | 9.133 | 1.00 | 51.60 |
| 2530 | C | ILE B | 119 | −0.076 | 17.038 | 13.734 | 1.00 | 38.62 |
| 2531 | O | ILE B | 119 | 0.261 | 16.123 | 14.483 | 1.00 | 47.55 |
| 2532 | N | TYR B | 120 | −0.818 | 18.068 | 14.125 | 1.00 | 32.69 |
| 2533 | CA | TYR B | 120 | −1.312 | 18.148 | 15.489 | 1.00 | 32.47 |
| 2534 | CB | TYR B | 120 | −0.950 | 19.474 | 16.123 | 1.00 | 16.42 |
| 2535 | CG | TYR B | 120 | 0.503 | 19.596 | 16.455 | 1.00 | 25.56 |
| 2536 | CD1 | TYR B | 120 | 1.419 | 20.056 | 15.515 | 1.00 | 35.91 |
| 2537 | CE1 | TYR B | 120 | 2.771 | 20.205 | 15.844 | 1.00 | −51.47 |
| 2538 | CD2 | TYR B | 120 | 0.966 | 19.275 | 17.728 | 1.00 | 32.05 |
| 2539 | CE2 | TYR B | 120 | 2.311 | 19.412 | 18.070 | 1.00 | 29.56 |
| 2540 | CZ | TYR B | 120 | 3.209 | 19.883 | 17.123 | 1.00 | 44.13 |
| 2541 | OH | TYR B | 120 | 4.537 | 20.063 | 17.438 | 1.00 | 29.40 |
| 2542 | C | TYR B | 120 | −2.806 | 18.001 | 15.490 | 1.00 | 33.00 |
| 2543 | O | TYR B | 120 | −3.484 | 18.535 | 14.625 | 1.00 | 54.53 |
| 2544 | N | TYR B | 121 | −3.322 | 17.277 | 16.467 | 1.00 | 31.54 |
| 2545 | CA | TYR B | 121 | −4.744 | 17.066 | 16.544 | 1.00 | 38.10 |
| 2546 | CB | TYR B | 121 | −5.068 | 15.570 | 16.402 | 1.00 | 34.72 |
| 2547 | CG | TYR B | 121 | −4.635 | 14.953 | 15.087 | 1.00 | 45.90 |
| 2548 | CD1 | TYR B | 121 | −3.293 | 14.656 | 14.855 | 1.00 | 43.35 |
| 2549 | CE1 | TYR B | 121 | −2.878 | 14.083 | 13.654 | 1.00 | 44.63 |
| 2550 | CD2 | TYR B | 121 | −5.570 | 14.659 | 14.076 | 1.00 | 45.04 |
| 2551 | CE2 | TYR B | 121 | −5.169 | 14.078 | 12.874 | 1.00 | 37.76 |
| 2552 | CZ | TYR B | 121 | −3.822 | 13.796 | 12.675 | 1.00 | 47.75 |
| 2553 | OH | TYR B | 121 | −3.411 | 13.225 | 11.502 | 1.00 | 43.41 |
| 2554 | C | TYR B | 121 | −5.272 | 17.579 | 17.864 | 1.00 | 51.86 |
| 2555 | 0 | TYR B | 121 | −4.735 | 17.246 | 18.927 | 1.00 | −65.44 |
| 2556 | N | LYS B | 122 | −6.314 | 18.403 | 17.794 | 1.00 | 51.19 |
| 2557 | CA | LYS B | 122 | −6.957 | 18.914 | 18.992 | 1.00 | 60.52 |
| 2558 | CB | LYS B | 122 | −7.037 | 20.435 | 18.969 | 1.00 | 58.03 |
| 2559 | CG | LYS B | 122 | −7.268 | 21.024 | 20.357 | 1.00 | 76.69 |
| 2560 | CD | LYS B | 122 | −7.727 | 22.472 | 20.312 | 1.00 | 76.94 |
| 2561 | CE | LYS B | 122 | −9.203 | 22.561 | 19.973 | 1.00 | 78.85 |
| 2562 | NZ | LYS B | 122 | −9.694 | 23.961 | 20.057 | 1.00 | 78.17 |
| 2563 | C | LYS B | 122 | −8.368 | 18.331 | 19.016 | 1.00 | 69.22 |
| 2564 | O | LYS B | 122 | −9.259 | 18.817 | 18.314 | 1.00 | 71.75 |
| 2565 | N | ASP B | 123 | −8.554 | 17.283 | 19.814 | 1.00 | 75.25 |
| 2566 | CA | ASP B | 123 | −9.840 | 16.611 | 19.936 | 1.00 | 81.83 |
| 2567 | CB | ASP B | 123 | −10.952 | 17.623 | 20.242 | 1.00 | 85.33 |
| 2568 | CG | ASP B | 123 | −10.809 | 18.253 | 21.618 | 1.00 | 86.97 |
| 2569 | OD1 | ASP B | 123 | −10.750 | 17.499 | 22.613 | 1.00 | 97.59 |
| 2570 | OD2 | ASP B | 123 | −10.764 | 19.501 | 21.703 | 1.00 | 80.57 |
| 2571 | C | ASP B | 123 | −10.172 | 15.841 | 18.660 | 1.00 | 85.87 |
| 2572 | O | ASP B | 123 | −11.183 | 16.107 | 18.002 | 1.00 | 90.87 |
| 2573 | N | GLY B | 124 | −9.310 | 14.889 | 18.314 | 1.00 | 83.44 |
| 2574 | CA | GLY B | 124 | −9.538 | 14.080 | 17.131 | 1.00 | 83.47 |
| 2575 | C | GLY B | 124 | −9.383 | 14.835 | 15.826 | 1.00 | 82.06 |
| 2576 | O | GLY B | 124 | −9.053 | 14.236 | 14.804 | 1.00 | 80.62 |
| 2577 | N | GLU B | 125 | −9.615 | 16.144 | 15.847 | 1.00 | 76.60 |
| 2578 | CA | GLU B | 125 | −9.479 | 16.951 | 14.640 | 1.00 | 69.95 |
| 2579 | CB | GLU B | 125 | −10.431 | 18.146 | 14.697 | 1.00 | 83.15 |
| 2580 | CG | GLU B | 125 | −11.903 | 17.778 | 14.854 | 1.00 | 99.12 |
| 2581 | CD | GLU B | 125 | −12.808 | 19.009 | 14.961 | 1.00 | 110.18 |
| 2582 | OE1 | GLU B | 125 | −12.287 | 20.153 | 14.909 | 1.00 | 115.12 |
| 2583 | OE2 | GLU B | 125 | −14.041 | 18.828 | 15.099 | 1.00 | 112.68 |
| 2584 | C | GLU B | 125 | −8.043 | 17.452 | 14.501 | 1.00 | 59.20 |
| 2585 | O | GLU B | 125 | −7.370 | 17.681 | 15.493 | 1.00 | 68.90 |
| 2586 | N | ALA B | 126 | −7.574 | 17.609 | 13.269 | 1.00 | 55.84 |
| 2587 | CA | ALA B | 126 | −6.221 | 18.112 | 13.032 | 1.00 | 49.26 |
| 2588 | CB | ALA B | 126 | −5.783 | 17.788 | 11.636 | 1.00 | 37.55 |
| 2589 | C | ALA B | 126 | −6.229 | 19.623 | 13.229 | 1.00 | 55.35 |
| 2590 | O | ALA B | 126 | −7.288 | 20.251 | 13.192 | 1.00 | 62.37 |
| 2591 | N | LEU B | 127 | −5.053 | 20.211 | 13.425 | 1.00 | 58.36 |
| 2592 | CA | LEU B | 127 | −4.968 | 21.648 | 13.652 | 1.00 | 56.69 |
| 2593 | CB | LEU B | 127 | −4.821 | 21.938 | 15.148 | 1.00 | 64.48 |
| 2594 | CG | LEU B | 127 | −4.606 | 23.411 | 15.496 | 1.00 | 71.37 |
| 2595 | CD1 | LEU B | 127 | −5.705 | 24.262 | 14.864 | 1.00 | 76.49 |
| 2596 | CD2 | LEU B | 127 | −4.590 | 23.573 | 17.012 | 1.00 | 74.87 |
| 2597 | C | LEU B | 127 | −3.837 | 22.314 | 12.895 | 1.00 | 59.25 |
| 2598 | O | LEU B | 127 | −3.979 | 23.463 | 12.449 | 1.00 | 65.52 |
| 2599 | N | LYS B | 128 | −2.710 | 21.619 | 12.760 | 1.00 | 39.64 |
| 2600 | CA | LYS B | 128 | −1.583 | 22.176 | 12.022 | 1.00 | 49.21 |
| 2601 | CB | LYS B | 128 | −0.695 | 23.067 | 12.911 | 1.00 | 55.50 |
| 2602 | CG | LYS B | 128 | −1.370 | 24.362 | 13.365 | 1.00 | 77.67 |
| 2603 | CD | LYS B | 128 | −0.363 | 25.479 | 13.615 | 1.00 | 85.20 |
| 2604 | CE | LYS B | 128 | −1.085 | 26.778 | 13.981 | 1.00 | 95.26 |
| 2605 | NZ | LYS B | 128 | −0.159 | 27.945 | 14.093 | 1.00 | 94.69 |
| 2606 | C | LYS B | 128 | −0.743 | 21.091 | 11.397 | 1.00 | 47.41 |
| 2607 | O | LYS B | 128 | −0.639 | 19.967 | 11.917 | 1.00 | 56.27 |
| 2608 | N | TYR B | 129 | −0.181 | 21.412 | 10.277 | 1.00 | 25.40 |
| 2609 | CA | TYR B | 129 | 0.682 | 20.491 | 9.551 | 1.00 | 31.17 |
| 2610 | CB | TYR B | 129 | −0.094 | 19.800 | 8.429 | 1.00 | 9.94 |
| 2611 | CG | TYR B | 129 | 0.773 | 18.963 | 7.516 | 1.00 | 7.68 |
| 2612 | CD1 | TYR B | 129 | 0.995 | 17.618 | 7.780 | 1.00 | 13.54 |
| 2613 | CE1 | TYR B | 129 | 1.788 | 16.850 | 6.948 | 1.00 | 15.62 |
| 2614 | CD2 | TYR B | 129 | 1.370 | 19.519 | 6.393 | 1.00 | 9.74 |
| 2615 | CE2 | TYR B | 129 | 2.164 | 18.759 | 5.558 | 1.00 | 6.56 |
| 2616 | CZ | TYR B | 129 | 2.369 | 17.425 | 5.839 | 1.00 | 23.46 |
| 2617 | OH | TYR B | 129 | 3.160 | 16.667 | 5.007 | 1.00 | 29.69 |
| 2618 | C | TYR B | 129 | 1.905 | 21.203 | 8.987 | 1.00 | 18.84 |
| 2619 | O | TYR B | 129 | 1.810 | 22.418 | 8.723 | 1.00 | 20.48 |
| 2620 | N | TRP B | 130 | 2.983 | 20.508 | 8.856 | 1.00 | 10.01 |
| 2621 | CA | TRP B | 130 | 4.195 | 21.121 | 8.376 | 1.00 | 26.80 |
| 2622 | CB | TRP B | 130 | 4.621 | 22.248 | 9.331 | 1.00 | 24.97 |
| 2623 | CG | TRP B | 130 | 5.657 | 23.141 | 8.763 | 1.00 | 34.86 |
| 2624 | CD2 | TRP B | 130 | 5.463 | 24.474 | 8.258 | 1.00 | 19.75 |
| 2625 | CE2 | TRP B | 130 | 6.718 | 24.933 | 7.789 | 1.00 | 18.96 |
| 2626 | CE3 | TRP B | 130 | 4.338 | 25.321 | 8.160 | 1.00 | 4.59 |
| 2627 | CD1 | TRP B | 130 | 6.995 | 22.854 | 8.577 | 1.00 | 46.79 |
| 2628 | NE1 | TRP B | 130 | 7.637 | 23.935 | 7.990 | 1.00 | 33.27 |
| 2629 | CZ2 | TRP B | 130 | 6.887 | 26.195 | 7.230 | 1.00 | 7.20 |
| 2630 | CZ3 | TRP B | 130 | 4.513 | 26.582 | 7.599 | 1.00 | 26.73 |
| 2631 | CH2 | TRP B | 130 | 5.778 | 27.003 | 7.141 | 1.00 | 33.91 |
| 2632 | C | TRP B | 130 | 5.204 | 19.990 | 8.359 | 1.00 | 28.67 |
| 2633 | O | TRP B | 130 | 5.511 | 19.385 | 9.388 | 1.00 | 37.85 |
| 2634 | N | TYR B | 131 | 5.686 | 19.697 | 7.164 | 1.00 | 25.88 |
| 2635 | CA | TYR B | 131 | 6.639 | 18.631 | 6.919 | 1.00 | 25.53 |
| 2636 | CB | TYR B | 131 | 7.327 | 18.864 | 5.599 | 1.00 | 12.69 |
| 2637 | CG | TYR B | 131 | 8.003 | 17.638 | 5.076 | 1.00 | 27.25 |
| 2638 | CD1 | TYR B | 131 | 7.258 | 16.534 | 4.672 | 1.00 | 27.89 |
| 2639 | CE1 | TYR B | 131 | 7.893 | 15.406 | 4.143 | 1.00 | 37.49 |
| 2640 | CD2 | TYR B | 131 | 9.385 | 17.589 | 4.949 | 1.00 | 47.62 |
| 2641 | CE2 | TYR B | 131 | 10.030 | 16.477 | 4.424 | 1.00 | 47.79 |
| 2642 | CZ | TYR B | 131 | 9.287 | 15.394 | 4.023 | 1.00 | 46.72 |
| 2643 | OH | TYR B | 131 | 9.948 | 14.315 | 3.487 | 1.00 | 44.87 |
| 2644 | C | TYR B | 131 | 7.699 | 18.490 | 7.988 | 1.00 | 41.53 |
| 2645 | O | TYR B | 131 | 7.730 | 17.491 | 8.714 | 1.00 | 45.44 |
| 2646 | N | GLU B | 132 | 8.584 | 19.481 | 8.058 | 1.00 | 41.35 |
| 2647 | CA | GLU B | 132 | 9.651 | 19.477 | 9.045 | 1.00 | 23.47 |
| 2648 | CB | GLU B | 132 | 10.631 | 20.595 | 8.746 | 1.00 | 26.69 |
| 2649 | CG | GLU B | 132 | 11.512 | 20.343 | 7.517 | 1.00 | 30.91 |
| 2650 | CD | GLU B | 132 | 12.674 | 19.419 | 7.819 | 1.00 | 59.01 |
| 2651 | OE1 | GLU B | 132 | 12.695 | 18.835 | 8.933 | 1.00 | 78.14 |
| 2652 | OE2 | GLU B | 132 | 13.555 | 19.276 | 6.939 | 1.00 | 45.30 |
| 2653 | C | GLU B | 132 | 9.017 | 19.678 | 10.410 | 1.00 | 30.80 |
| 2654 | O | GLU B | 132 | 7.926 | 20.252 | 10.503 | 1.00 | 15.18 |
| 2655 | N | ASN B | 133 | 9.673 | 19.198 | 11.466 | 1.00 | 38.68 |
| 2656 | CA | ASN B | 133 | 9.078 | 19.340 | 12.785 | 1.00 | 35.69 |
| 2657 | CB | ASN B | 133 | 9.969 | 18.765 | 13.884 | 1.00 | 24.76 |
| 2658 | CG | ASN B | 133 | 9.165 | 18.350 | 15.126 | 1.00 | 52.21 |
| 2659 | OD1 | ASN B | 133 | 8.183 | 19.018 | 15.524 | 1.00 | 35.03 |
| 2660 | ND2 | ASN B | 133 | 9.582 | 17.243 | 15.745 | 1.00 | 43.95 |
| 2661 | C | ASN B | 133 | 8.857 | 20.813 | 13.030 | 1.00 | 29.20 |
| 2662 | O | ASN B | 133 | 9.651 | 21.644 | 12.587 | 1.00 | 43.35 |
| 2663 | N | HIS B | 134 | 7.770 | 21.132 | 13.718 | 1.00 | 15.66 |
| 2664 | CA | HIS B | 134 | 7.435 | 22.506 | 14.022 | 1.00 | 21.19 |
| 2665 | CB | HIS B | 134 | 6.522 | 23.054 | 12.927 | 1.00 | 41.57 |
| 2666 | CG | HIS B | 134 | 5.268 | 22.256 | 12.734 | 1.00 | 45.84 |
| 2667 | CD2 | HIS B | 134 | 3.972 | 22.543 | 12.990 | 1.00 | 30.22 |
| 2668 | ND1 | HIS B | 134 | 5.280 | 20.969 | 12.228 | 1.00 | 29.02 |
| 2669 | CE1 | HIS B | 134 | 4.037 | 20.507 | 12.183 | 1.00 | 45.78 |
| 2670 | NE2 | HIS B | 134 | 3.230 | 21.443 | 12.640 | 1.00 | 39.55 |
| 2671 | C | HIS B | 134 | 6.733 | 22.587 | 15.380 | 1.00 | 30.10 |
| 2672 | 0 | HIS B | 134 | 6.072 | 21.631 | 15.826 | 1.00 | 22.57 |
| 2673 | N | ASN B | 135 | 6.871 | 23.731 | 16.043 | 1.00 | 35.59 |
| 2674 | CA | ASN B | 135 | 6.263 | 23.933 | 17.355 | 1.00 | 43.05 |
| 2675 | CB | ASN B | 135 | 7.182 | 24.741 | 18.303 | 1.00 | 49.56 |
| 2676 | CG | ASN B | 135 | 8.585 | 24.138 | 18.481 | 1.00 | 62.83 |
| 2677 | OD1 | ASN B | 135 | 8.717 | 22.936 | 18.749 | 1.00 | 59.97 |
| 2678 | ND2 | ASN B | 135 | 9.614 | 24.991 | 18.356 | 1.00 | 50.35 |
| 2679 | C | ASN B | 135 | 5.006 | 24.761 | 17.162 | 1.00 | 31.65 |
| 2680 | O | ASN B | 135 | 5.024 | 25.700 | 16.384 | 1.00 | 53.46 |
| 2681 | N | ILE B | 136 | 3.910 | 24.406 | 17.820 | 1.00 | 39.40 |
| 2682 | CA | ILE B | 136 | 2.720 | 25.244 | 17.749 | 1.00 | 46.75 |
| 2683 | CB | ILE B | 136 | 1.440 | 24.484 | 18.081 | 1.00 | 51.16 |
| 2684 | CG2 | ILE B | 136 | 0.254 | 25.448 | 18.105 | 1.00 | 27.02 |
| 2685 | CG1 | ILE B | 136 | 1.232 | 23.368 | 17.065 | 1.00 | 48.68 |
| 2686 | CD1 | ILE B | 136 | −0.057 | 22.599 | 17.259 | 1.00 | 57.62 |
| 2687 | C | ILE B | 136 | 2.922 | 26.304 | 18.834 | 1.00 | 57.38 |
| 2688 | O | ILE B | 136 | 2.996 | 25.995 | 20.026 | 1.00 | 38.50 |
| 2689 | N | SER B | 137 | 3.035 | 27.554 | 18.409 | 1.00 | 62.66 |
| 2690 | CA | SER B | 137 | 3.238 | 28.653 | 19.333 | 1.00 | 62.59 |
| 2691 | CB | SER B | 137 | 4.495 | 29.443 | 18.941 | 1.00 | 62.06 |
| 2692 | OG | SER B | 137 | 4.789 | 30.448 | 19.899 | 1.00 | 70.77 |
| 2693 | C | SER B | 137 | 2.015 | 29.557 | 19.314 | 1.00 | 54.31 |
| 2694 | O | SER B | 137 | 1.675 | 30.140 | 18.295 | 1.00 | 61.87 |
| 2695 | N | ILE B | 138 | 1.344 | 29.665 | 20.450 | 1.00 | 57.19 |
| 2696 | CA | ILE B | 138 | 0.167 | 30.511 | 20.551 | 1.00 | 64.51 |
| 2697 | CB | ILE B | 138 | −1.060 | 29.702 | 20.970 | 1.00 | 66.59 |
| 2698 | CG2 | ILE B | 138 | −2.289 | 30.588 | 20.965 | 1.00 | 62.45 |
| 2699 | CG1 | ILE B | 138 | −1.244 | 28.528 | 20.011 | 1.00 | 72.23 |
| 2700 | CD1 | ILE B | 138 | −2.439 | 27.657 | 20.338 | 1.00 | 72.19 |
| 2701 | C | ILE B | 138 | 0.409 | 31.600 | 21.581 | 1.00 | 70.00 |
| 2702 | O | ILE B | 138 | 0.207 | 31.396 | 22.777 | 1.00 | 80.22 |
| 2703 | N | THR B | 139 | 0.846 | 32.760 | 21.100 | 1.00 | 75.14 |
| 2704 | CA | THR B | 139 | 1.143 | 33.914 | 21.951 | 1.00 | 76.04 |
| 2705 | CB | THR B | 139 | 1.419 | 35.149 | 21.086 | 1.00 | 66.84 |
| 2706 | OG1 | THR B | 139 | 0.347 | 35.322 | 20.153 | 1.00 | 70.10 |
| 2707 | CG2 | THR B | 139 | 2.720 | 34.969 | 20.312 | 1.00 | 62.07 |
| 2708 | C | THR B | 139 | 0.064 | 34.246 | 22.993 | 1.00 | 77.29 |
| 2709 | O | THR B | 139 | 0.333 | 34.206 | 24.190 | 1.00 | 88.78 |
| 2710 | N | ASN B | 140 | −1.142 | 34.598 | 22.553 | 1.00 | 78.71 |
| 2711 | CA | ASN B | 140 | −2.244 | 34.893 | 23.485 | 1.00 | 86.24 |
| 2712 | CB | ASN B | 140 | −2.994 | 36.169 | 23.082 | 1.00 | 96.34 |
| 2713 | CG | ASN B | 140 | −2.195 | 37.465 | 23.304 | 1.00 | 113.86 |
| 2714 | OD1 | ASN B | 140 | −2.591 | 38.486 | 22.747 | 1.00 | 123.78 |
| 2715 | ND2 | ASN B | 140 | −1.120 | 37.473 | 24.100 | 1.00 | 121.56 |
| 2716 | C | ASN B | 140 | −3.245 | 33.716 | 23.456 | 1.00 | 84.41 |
| 2717 | O | ASN B | 140 | −3.962 | 33.521 | 22.474 | 1.00 | 92.35 |
| 2718 | N | ALA B | 141 | −3.298 | 32.944 | 24.536 | 1.00 | 74.85 |
| 2719 | CA | ALA B | 141 | −4.186 | 31.781 | 24.642 | 1.00 | 70.00 |
| 2720 | CB | ALA B | 141 | −3.768 | 30.931 | 25.819 | 1.00 | 60.72 |
| 2721 | C | ALA B | 141 | −5.679 | 32.080 | 24.744 | 1.00 | 73.54 |
| 2722 | O | ALA B | 141 | −6.083 | 33.215 | 25.000 | 1.00 | 71.12 |
| 2723 | N | THR B | 142 | −6.486 | 31.033 | 24.557 | 1.00 | 79.19 |
| 2724 | CA | THR B | 142 | −7.945 | 31.135 | 24.602 | 1.00 | 81.65 |
| 2725 | CB | THR B | 142 | −8.550 | 31.294 | 23.191 | 1.00 | 87.55 |
| 2726 | OG1 | THR B | 142 | −7.856 | 32.324 | 22.475 | 1.00 | 87.31 |
| 2727 | CG2 | THR B | 142 | −10.032 | 31.647 | 23.292 | 1.00 | 80.50 |
| 2728 | C | THR B | 142 | −8.556 | 29.872 | 25.196 | 1.00 | 81.42 |
| 2729 | O | THR B | 142 | −8.001 | 28.782 | 25.063 | 1.00 | 80.47 |
| 2730 | N | VAL B | 143 | −9.716 | 30.022 | 25.828 | 1.00 | 79.19 |
| 2731 | CA | VAL B | 143 | −10.385 | 28.881 | 26.436 | 1.00 | 80.64 |
| 2732 | CB | VAL B | 143 | −11.681 | 29.314 | 27.154 | 1.00 | 85.23 |
| 2733 | CG1 | VAL B | 143 | −12.667 | 29.899 | 26.146 | 1.00 | 91.61 |
| 2734 | CG2 | VAL B | 143 | −12.293 | 28.123 | 27.880 | 1.00 | 91.42 |
| 2735 | C | VAL B | 143 | −10.728 | 27.848 | 25.370 | 1.00 | 78.44 |
| 2736 | O | VAL B | 143 | −10.878 | 26.659 | 25.659 | 1.00 | 67.92 |
| 2737 | N | GLU B | 144 | −10.847 | 28.317 | 24.132 | 1.00 | 87.88 |
| 2738 | CA | GLU B | 144 | −11.173 | 27.438 | 23.019 | 1.00 | 94.42 |
| 2739 | CB | GLU B | 144 | −11.546 | 28.261 | 21.775 | 1.00 | 103.89 |
| 2740 | CG | GLU B | 144 | −12.116 | 27.439 | 20.605 | 1.00 | 127.47 |
| 2741 | CD | GLU B | 144 | −13.460 | 26.781 | 20.925 | 1.00 | 145.47 |
| 2742 | OE1 | GLU B | 144 | −14.018 | 27.052 | 22.011 | 1.00 | 154.66 |
| 2743 | OE2 | GLU B | 144 | −13.960 | 25.995 | 20.087 | 1.00 | 153.18 |
| 2744 | C | GLU B | 144 | −9.977 | 26.546 | 22.721 | 1.00 | 87.49 |
| 2745 | O | GLU B | 144 | −10.137 | 25.385 | 22.328 | 1.00 | 92.98 |
| 2746 | N | ASP B | 145 | −8.777 | 27.086 | 22.919 | 1.00 | 73.99 |
| 2747 | CA | ASP B | 145 | −7.557 | 26.336 | 22.674 | 1.00 | 73.40 |
| 2748 | CB | ASP B | 145 | −6.348 | 27.253 | 22.815 | 1.00 | 76.84 |
| 2749 | CG | ASP B | 145 | −6.159 | 28.159 | 21.614 | 1.00 | 84.39 |
| 2750 | OD1 | ASP B | 145 | −6.061 | 27.632 | 20.481 | 1.00 | 89.54 |
| 2751 | OD2 | ASP B | 145 | −6.101 | 29.394 | 21.801 | 1.00 | 78.78 |
| 2752 | C | ASP B | 145 | −7.434 | 25.135 | 23.618 | 1.00 | 72.65 |
| 2753 | O | ASP B | 145 | −6.468 | 24.371 | 23.546 | 1.00 | 66.12 |
| 2754 | N | SER B | 146 | −8.423 | 24.971 | 24.496 | 1.00 | 68.40 |
| 2755 | CA | SER B | 146 | −8.445 | 23.857 | 25.441 | 1.00 | 59.96 |
| 2756 | CB | SER B | 146 | −9.433 | 24.128 | 26.579 | 1.00 | 59.40 |
| 2757 | OG | SER B | 146 | −8.959 | 25.158 | 27.435 | 1.00 | 82.76 |
| 2758 | C | SER B | 146 | −8.862 | 22.590 | 24.725 | 1.00 | 55.02 |
| 2759 | O | SER B | 146 | −9.531 | 22.649 | 23.703 | 1.00 | 74.05 |
| 2760 | N | GLY B | 147 | −8.462 | 21.447 | 25.267 | 1.00 | 55.86 |
| 2761 | CA | GLY B | 147 | −8.811 | 20.174 | 24.662 | 1.00 | 55.22 |
| 2762 | C | GLY B | 147 | −7.680 | 19.168 | 24.739 | 1.00 | 55.94 |
| 2763 | O | GLY B | 147 | −6.624 | 19.453 | 25.308 | 1.00 | 65.07 |
| 2764 | N | THR B | 148 | −7.895 | 17.984 | 24.173 | 1.00 | 51.77 |
| 2765 | CA | THR B | 148 | −6.871 | 16.945 | 24.186 | 1.00 | 56.97 |
| 2766 | CB | THR B | 148 | −7.493 | 15.537 | 24.299 | 1.00 | 62.53 |
| 2767 | OG1 | THR B | 148 | −7.822 | 15.054 | 22.992 | 1.00 | 93.22 |
| 2768 | CG2 | THR B | 148 | −8.769 | 15.581 | 25.130 | 1.00 | 61.98 |
| 2769 | C | THR B | 148 | −6.061 | 17.028 | 22.895 | 1.00 | 50.21 |
| 2770 | O | THR B | 148 | −6.605 | 16.990 | 21.803 | 1.00 | 61.31 |
| 2771 | N | TYR B | 149 | −4.752 | 17.167 | 23.026 | 1.00 | 47.96 |
| 2772 | CA | TYR B | 149 | −3.873 | 17.263 | 21.870 | 1.00 | 36.15 |
| 2773 | CB | TYR B | 149 | −2.907 | 18.438 | 22.008 | 1.00 | 39.15 |
| 2774 | CG | TYR B | 149 | −3.504 | 19.814 | 21.873 | 1.00 | 32.57 |
| 2775 | CD1 | TYR B | 149 | −4.170 | 20.418 | 22.932 | 1.00 | 43.98 |
| 2776 | CE1 | TYR B | 149 | −4.695 | 21.704 | 22.815 | 1.00 | 43.58 |
| 2777 | CD2 | TYR B | 149 | −3.382 | 20.525 | 20.687 | 1.00 | 33.74 |
| 2778 | CE2 | TYR B | 149 | −3.909 | 21.810 | 20.553 | 1.00 | 35.76 |
| 2779 | CZ | TYR B | 149 | −4.564 | 22.396 | 21.621 | 1.00 | 41.82 |
| 2780 | OH | TYR B | 149 | −5.089 | 23.668 | 21.494 | 1.00 | 40.18 |
| 2781 | C | TYR B | 149 | −3.028 | 16.013 | 21.730 | 1.00 | 38.28 |
| 2782 | O | TYR B | 149 | −2.944 | 15.191 | 22.651 | 1.00 | 37.42 |
| 2783 | N | TYR B | 150 | −2.386 | 15.893 | 20.573 | 1.00 | 28.35 |
| 2784 | CA | TYR B | 150 | −1.500 | 14.776 | 20.287 | 1.00 | 32.70 |
| 2785 | CB | TYR B | 150 | −2.226 | 13.421 | 20.440 | 1.00 | 46.79 |
| 2786 | CG | TYR B | 150 | −3.180 | 13.018 | 19.328 | 1.00 | 40.91 |
| 2787 | CD1 | TYR B | 150 | −2.710 | 12.397 | 18.168 | 1.00 | 38.70 |
| 2788 | CE1 | TYR B | 150 | −3.588 | 12.007 | 17.150 | 1.00 | 49.78 |
| 2789 | CD2 | TYR B | 150 | −4.556 | 13.245 | 19.447 | 1.00 | 29.49 |
| 2790 | CE2 | TYR B | 150 | −5.440 | 12.862 | 18.436 | 1.00 | 54.83 |
| 2791 | CZ | TYR B | 150 | −4.950 | 12.245 | 17.294 | 1.00 | 51.46 |
| 2792 | OH | TYR B | 150 | −5.828 | 11.857 | 16.310 | 1.00 | 68.38 |
| 2793 | C | TYR B | 150 | −1.002 | 14.971 | 18.880 | 1.00 | 30.40 |
| 2794 | 0 | TYR B | 150 | −1.718 | 15.477 | 18.028 | 1.00 | 49.73 |
| 2795 | N | CYS B | 151 | 0.241 | 14.596 | 18.645 | 1.00 | 25.26 |
| 2796 | CA | CYS B | 151 | 0.808 | 14.746 | 17.326 | 1.00 | 39.72 |
| 2797 | C | CYS B | 151 | 1.175 | 13.410 | 16.699 | 1.00 | 37.05 |
| 2798 | 0 | CYS B | 151 | 1.270 | 12.395 | 17.383 | 1.00 | 49.88 |
| 2799 | CB | CYS B | 151 | 2.040 | 15.652 | 17.399 | 1.00 | 44.87 |
| 2800 | SG | CYS B | 151 | 3.415 | 15.099 | 18.482 | 1.00 | 47.69 |
| 2801 | N | THR B | 152 | 1.380 | 13.419 | 15.387 | 1.00 | 34.36 |
| 2802 | CA | THR B | 152 | 1.753 | 12.217 | 14.648 | 1.00 | 40.90 |
| 2803 | CB | THR B | 152 | 0.641 | 11.715 | 13.712 | 1.00 | 46.04 |
| 2804 | OG1 | THR B | 152 | 0.435 | 12.666 | 12.656 | 1.00 | 59.44 |
| 2805 | CG2 | THR B | 152 | −0.652 | 11.514 | 14.487 | 1.00 | 54.68 |
| 2806 | C | THR B | 152 | 2.906 | 12.646 | 13.784 | 1.00 | 39.86 |
| 2807 | O | THR B | 152 | 3.063 | 13.845 | 13.500 | 1.00 | 42.60 |
| 2808 | N | GLY B | 153 | 3.707 | 11.680 | 13.355 | 1.00 | 28.17 |
| 2809 | CA | GLY B | 153 | 4.846 | 12.024 | 12.540 | 1.00 | 26.12 |
| 2810 | C | GLY B | 153 | 5.647 | 10.818 | 12.112 | 1.00 | 33.14 |
| 2811 | O | GLY B | 153 | 5.622 | 9.753 | 12.734 | 1.00 | 34.08 |
| 2812 | N | LYS B | 154 | 6.365 | 10.998 | 11.021 | 1.00 | 17.31 |
| 2813 | CA | LYS B | 154 | 7.188 | 9.959 | 10.471 | 1.00 | 31.41 |
| 2814 | CB | LYS B | 154 | 7.185 | 10.086 | 8.949 | 1.00 | 56.81 |
| 2815 | CG | LYS B | 154 | 8.472 | 9.671 | 8.248 | 1.00 | 78.50 |
| 2816 | CD | LYS B | 154 | 8.399 | 10.064 | 6.771 | 1.00 | 85.12 |
| 2817 | CE | LYS B | 154 | 9.733 | 9.881 | 6.064 | 1.00 | 88.41 |
| 2818 | NZ | LYS B | 154 | 9.649 | 10.343 | 4.653 | 1.00 | 64.75 |
| 2819 | C | LYS B | 154 | 8.597 | 10.101 | 11.028 | 1.00 | 44.08 |
| 2820 | O | LYS B | 154 | 9.266 | 11.116 | 10.811 | 1.00 | 51.89 |
| 2821 | N | VAL B | 155 | 9.027 | 9.078 | 11.762 | 1.00 | 46.05 |
| 2822 | CA | VAL B | 155 | 10.362 | 9.047 | 12.345 | 1.00 | 43.86 |
| 2823 | CB | VAL B | 155 | 10.321 | 8.903 | 13.878 | 1.00 | 37.35 |
| 2824 | CG1 | VAL B | 155 | 11.732 | 8.926 | 14.416 | 1.00 | 21.63 |
| 2825 | CG2 | VAL B | 155 | 9.487 | 10.030 | 14.499 | 1.00 | 48.98 |
| 2826 | C | VAL B | 155 | 11.035 | 7.823 | 11.779 | 1.00 | 41.23 |
| 2827 | O | VAL B | 155 | 10.560 | 6.716 | 11.989 | 1.00 | 37.63 |
| 2828 | N | TRP B | 156 | 12.129 | 8.012 | 11.057 | 1.00 | 52.20 |
| 2829 | CA | TRP B | 156 | 12.824 | 6.880 | 10.454 | 1.00 | 74.29 |
| 2830 | CB | TRP B | 156 | 13.196 | 5.838 | 11.510 | 1.00 | 65.13 |
| 2831 | CG | TRP B | 156 | 14.467 | 6.147 | 12.211 | 1.00 | 70.49 |
| 2832 | CD2 | TRP B | 156 | 15.771 | 6.221 | 11.629 | 1.00 | 54.80 |
| 2833 | CE2 | TRP B | 156 | 16.670 | 6.585 | 12.646 | 1.00 | 64.98 |
| 2834 | CE3 | TRP B | 156 | 16.252 | 6.012 | 10.334 | 1.00 | 50.71 |
| 2835 | CD1 | TRP B | 156 | 14.631 | 6.458 | 13.539 | 1.00 | 71.39 |
| 2836 | NE1 | TRP B | 156 | 15.954 | 6.725 | 13.804 | 1.00 | 63.30 |
| 2837 | CZ2 | TRP B | 156 | 18.051 | 6.752 | 12.415 | 1.00 | 62.21 |
| 2838 | CZ3 | TRP B | 156 | 17.624 | 6.179 | 10.101 | 1.00 | 67.16 |
| 2839 | CH2 | TRP B | 156 | 18.500 | 6.545 | 11.141 | 1.00 | 53.68 |
| 2840 | C | TRP B | 156 | 12.015 | 6.201 | 9.347 | 1.00 | 90.81 |
| 2841 | O | TRP B | 156 | 11.962 | 4.971 | 9.263 | 1.00 | 111.59 |
| 2842 | N | GLN B | 157 | 11.389 | 7.013 | 8.506 | 1.00 | 88.48 |
| 2843 | CA | GLN B | 157 | 10.608 | 6.526 | 7.382 | 1.00 | 85.04 |
| 2844 | CB | GLN B | 157 | 11.461 | 5.575 | 6.536 | 1.00 | 110.53 |
| 2845 | CG | GLN B | 157 | 10.930 | 5.324 | 5.138 | 1.00 | 124.62 |
| 2846 | CD | GLN B | 157 | 11.824 | 4.383 | 4.337 | 1.00 | 129.56 |
| 2847 | OE1 | GLN B | 157 | 12.951 | 4.080 | 4.749 | 1.00 | 127.90 |
| 2848 | NE2 | GLN B | 157 | 11.329 | 3.926 | 3.183 | 1.00 | 123.80 |
| 2849 | C | GLN B | 157 | 9.311 | 5.842 | 7.816 | 1.00 | 78.48 |
| 2850 | O | GLN B | 157 | 8.544 | 5.392 | 6.977 | 1.00 | 77.35 |
| 2851 | N | LEU B | 158 | 9.074 | 5.760 | 9.122 | 1.00 | 70.67 |
| 2852 | CA | LEU B | 158 | 7.849 | 5.147 | 9.635 | 1.00 | 70.41 |
| 2853 | CB | LEU B | 158 | 8.170 | 4.049 | 10.637 | 1.00 | 81.95 |
| 2854 | CG | LEU B | 158 | 8.646 | 2.722 | 10.055 | 1.00 | 86.23 |
| 2855 | CD1 | LEU B | 158 | 8.865 | 1.715 | 11.174 | 1.00 | 105.28 |
| 2856 | CD2 | LEU B | 158 | 7.602 | 2.204 | 9.081 | 1.00 | 94.27 |
| 2857 | C | LEU B | 158 | 6.998 | 6.203 | 10.313 | 1.00 | 70.44 |
| 2858 | O | LEU B | 158 | 7.499 | 7.275 | 10.632 | 1.00 | 80.05 |
| 2859 | N | ASP B | 159 | 5.721 | 5.904 | 10.537 | 1.00 | 67.41 |
| 2860 | CA | ASP B | 159 | 4.837 | 6.880 | 11.171 | 1.00 | 82.00 |
| 2861 | CB | ASP B | 159 | 3.519 | 7.001 | 10.392 | 1.00 | 86.28 |
| 2862 | CG | ASP B | 159 | 3.736 | 7.375 | 8.938 | 1.00 | 106.10 |
| 2863 | OD1 | ASP B | 159 | 4.285 | 8.469 | 8.664 | 1.00 | 117.95 |
| 2864 | OD2 | ASP B | 159 | 3.361 | 6.564 | 8.067 | 1.00 | 115.39 |
| 2865 | C | ASP B | 159 | 4.547 | 6.495 | 12.611 | 1.00 | 77.19 |
| 2866 | O | ASP B | 159 | 4.590 | 5.316 | 12.962 | 1.00 | 77.19 |
| 2867 | N | TYR B | 160 | 4.274 | 7.492 | 13.448 | 1.00 | 65.57 |
| 2868 | CA | TYR B | 160 | 3.972 | 7.232 | 14.842 | 1.00 | 48.77 |
| 2869 | CB | TYR B | 160 | 5.244 | 7.273 | 15.670 | 1.00 | 37.12 |
| 2870 | CG | TYR B | 160 | 6.332 | 6.371 | 15.148 | 1.00 | 34.46 |
| 2871 | CD1 | TYR B | 160 | 7.199 | 6.797 | 14.134 | 1.00 | 37.79 |
| 2872 | CE1 | TYR B | 160 | 8.195 | 5.961 | 13.645 | 1.00 | 56.87 |
| 2873 | CD2 | TYR B | 160 | 6.490 | 5.086 | 15.661 | 1.00 | 19.69 |
| 2874 | CE2 | TYR B | 160 | 7.486 | 4.233 | 15.179 | 1.00 | 40.99 |
| 2875 | CZ | TYR B | 160 | 8.341 | 4.674 | 14.172 | 1.00 | 60.40 |
| 2876 | OH | TYR B | 160 | 9.343 | 3.841 | 13.702 | 1.00 | 70.89 |
| 2877 | C | TYR B | 160 | 3.000 | 8.266 | 15.364 | 1.00 | 54.13 |
| 2878 | O | TYR B | 160 | 2.718 | 9.265 | 14.687 | 1.00 | 60.44 |
| 2879 | N | GLU B | 161 | 2.496 | 8.022 | 16.569 | 1.00 | 47.74 |
| 2880 | CA | GLU B | 161 | 1.546 | 8.926 | 17.193 | 1.00 | 48.57 |
| 2881 | CB | GLU B | 161 | 0.133 | 8.387 | 16.988 | 1.00 | 55.17 |
| 2882 | CG | GLU B | 161 | −0.965 | 9.106 | 17.748 | 1.00 | 80.84 |
| 2883 | CD | GLU B | 161 | −2.360 | 8.757 | 17.226 | 1.00 | 93.70 |
| 2884 | OE1 | GLU B | 161 | −3.352 | 9.054 | 17.932 | 1.00 | 99.33 |
| 2885 | OE2 | GLU B | 161 | −2.465 | 8.198 | 16.106 | 1.00 | 101.86 |
| 2886 | C | GLU B | 161 | 1.872 | 9.038 | 18.671 | 1.00 | 56.41 |
| 2887 | O | GLU B | 161 | 2.080 | 8.028 | 19.340 | 1.00 | 58.09 |
| 2888 | N | SER B | 162 | 1.928 | 10.268 | 19.173 | 1.00 | 61.15 |
| 2889 | CA | SER B | 162 | 2.240 | 10.515 | 20.578 | 1.00 | 70.01 |
| 2890 | CB | SER B | 162 | 2.700 | 11.965 | 20.771 | 1.00 | 82.47 |
| 2891 | OG | SER B | 162 | 1.646 | 12.889 | 20.523 | 1.00 | 75.43 |
| 2892 | C | SER B | 162 | 1.046 | 10.258 | 21.481 | 1.00 | 69.94 |
| 2893 | O | SER B | 162 | −0.083 | 10.163 | 21.017 | 1.00 | 70.40 |
| 2894 | N | GLU B | 163 | 1.301 | 10.133 | 22.776 | 1.00 | 76.24 |
| 2895 | CA | GLU B | 163 | 0.223 | 9.924 | 23.728 | 1.00 | 74.01 |
| 2896 | CB | GLU B | 163 | 0.785 | 9.585 | 25.106 | 1.00 | 83.42 |
| 2897 | CG | GLU B | 163 | 1.437 | 8.222 | 25.201 | 1.00 | 108.95 |
| 2898 | CD | GLU B | 163 | 0.421 | 7.096 | 25.165 | 1.00 | 124.63 |
| 2899 | OE1 | GLU B | 163 | −0.463 | 7.070 | 26.051 | 1.00 | 126.10 |
| 2900 | OE2 | GLU B | 163 | 0.507 | 6.239 | 24.255 | 1.00 | 137.68 |
| 2901 | C | GLU B | 163 | −0.517 | 11.246 | 23.809 | 1.00 | 64.66 |
| 2902 | O | GLU B | 163 | 0.096 | 12.303 | 23.836 | 1.00 | 78.89 |
| 2903 | N | PRO B | 164 | −1 .848 | 11.211 | 23.829 | 1.00 | 49.91 |
| 2904 | CD | PRO B | 164 | −2.763 | 10.066 | 23.886 | 1.00 | 45.53 |
| 2905 | CA | PRO B | 164 | −2.580 | 12.477 | 23.912 | 1.00 | 49.79 |
| 2906 | CB | PRO B | 164 | −4.040 | 12.042 | 23.913 | 1.00 | 50.14 |
| 2907 | CG | PRO B | 164 | −3.988 | 10.692 | 24.532 | 1.00 | 54.43 |
| 2908 | C | PRO B | 164 | −2.210 | 13.229 | 25.177 | 1.00 | 52.68 |
| 2909 | O | PRO B | 164 | −1.676 | 12.650 | 26.121 | 1.00 | 55.25 |
| 2910 | N | LEU B | 165 | −2.496 | 14.523 | 25.187 | 1.00 | 46.01 |
| 2911 | CA | LEU B | 165 | −2.195 | 15.363 | 26.330 | 1.00 | 38.63 |
| 2912 | CB | LEU B | 165 | −0.862 | 16.080 | 26.112 | 1.00 | 42.39 |
| 2913 | CG | LEU B | 165 | −0.390 | 17.012 | 27.232 | 1.00 | 34.72 |
| 2914 | CD1 | LEU B | 165 | 0.058 | 16.181 | 28.423 | 1.00 | 36.79 |
| 2915 | CD2 | LEU B | 165 | 0.765 | 17.879 | 26.732 | 1.00 | 31.18 |
| 2916 | C | LEU B | 165 | −3.306 | 16.390 | 26.491 | 1.00 | 41.11 |
| 2917 | O | LEU B | 165 | −3.561 | 17.181 | 25.597 | 1.00 | 47.00 |
| 2918 | N | ASN B | 166 | −3.983 | 16.369 | 27.626 | 1.00 | 47.91 |
| 2919 | CA | ASN B | 166 | −5.044 | 17.332 | 27.852 | 1.00 | 62.56 |
| 2920 | CB | ASN B | 166 | −5.919 | 16.892 | 29.031 | 1.00 | 89.42 |
| 2921 | CG | ASN B | 166 | −6.897 | 15.795 | 28.644 | 1.00 | 111.40 |
| 2922 | OD1 | ASN B | 166 | −7.511 | 15.874 | 27.581 | 1.00 | 117.56 |
| 2923 | ND2 | ASN B | 166 | −7.069 | 14.782 | 29.490 | 1.00 | 128.12 |
| 2924 | C | ASN B | 166 | −4.419 | 18.685 | 28.139 | 1.00 | 63.19 |
| 2925 | O | ASN B | 166 | −3.385 | 18.758 | 28.798 | 1.00 | 75.62 |
| 2926 | N | ILE B | 167 | −5.028 | 19.743 | 27.614 | 1.00 | 55.85 |
| 2927 | CA | ILE B | 167 | −4.547 | 21.103 | 27.837 | 1.00 | 50.63 |
| 2928 | CB | ILE B | 167 | −3.775 | 21.642 | 26.625 | 1.00 | 41.65 |
| 2929 | CG2 | ILE B | 167 | −3.644 | 23.158 | 26.704 | 1.00 | 27.67 |
| 2930 | CG1 | ILE B | 167 | −2.398 | 20.981 | 26.572 | 1.00 | 49.69 |
| 2931 | CD1 | ILE B | 167 | −1.576 | 21.401 | 25.373 | 1.00 | 71.73 |
| 2932 | C | ILE B | 167 | −5.719 | 22.018 | 28.127 | 1.00 | 52.51 |
| 2933 | O | ILE B | 167 | −6.636 | 22.141 | 27.328 | 1.00 | 54.25 |
| 2934 | N | THR B | 168 | −5.677 | 22.669 | 29.279 | 1.00 | 62.54 |
| 2935 | CA | THR B | 168 | −6.762 | 23.552 | 29.672 | 1.00 | 73.12 |
| 2936 | CB | THR B | 168 | −7.371 | 23.090 | 30.995 | 1.00 | 77.82 |
| 2937 | OG1 | THR B | 168 | −7.229 | 21.668 | 31.116 | 1.00 | 71.03 |
| 2938 | CG2 | THR B | 168 | −8.847 | 23.472 | 31.049 | 1.00 | 80.41 |
| 2939 | C | THR B | 168 | −6.329 | 25.002 | 29.841 | 1.00 | 79.24 |
| 2940 | O | THR B | 168 | −5.347 | 25.294 | 30.526 | 1.00 | 81.73 |
| 2941 | N | VAL B | 169 | −7.070 | 25.907 | 29.214 | 1.00 | 81.68 |
| 2942 | CA | VAL B | 169 | −6.792 | 27.336 | 29.302 | 1.00 | 82.56 |
| 2943 | CB | VAL B | 169 | −6.787 | 27.996 | 27.916 | 1.00 | 80.42 |
| 2944 | CG1 | VAL B | 169 | −6.538 | 29.487 | 28.048 | 1.00 | 71.55 |
| 2945 | CG2 | VAL B | 169 | −5.731 | 27.352 | 27.041 | 1.00 | 82.46 |
| 2946 | C | VAL B | 169 | −7.937 | 27.908 | 30.115 | 1.00 | 89.49 |
| 2947 | O | VAL B | 169 | −9.098 | 27.841 | 29.696 | 1.00 | 87.82 |
| 2948 | N | ILE B | 170 | −7.625 | 28.469 | 31.277 | 1.00 | 94.48 |
| 2949 | CA | ILE B | 170 | −8.668 | 28.995 | 32.143 | 1.00 | 101.94 |
| 2950 | CB | ILE B | 170 | −8.332 | 28.730 | 33.631 | 1.00 | 98.82 |
| 2951 | CG2 | ILE B | 170 | −8.574 | 27.262 | 33.966 | 1.00 | 99.33 |
| 2952 | CG1 | ILE B | 170 | −6.876 | 29.092 | 33.912 | 1.00 | 103.85 |
| 2953 | CD1 | ILE B | 170 | −6.453 | 28.836 | 35.347 | 1.00 | 102.26 |
| 2954 | C | ILE B | 170 | −9.027 | 30.467 | 31.967 | 1.00 | 106.09 |
| 2955 | O | ILE B | 170 | −8.162 | 31.307 | 31.727 | 1.00 | 103.79 |
| 2956 | N | LYS B | 171 | −10.326 | 30.715 | 32.066 | 1.00 | 115.25 |
| 2957 | CA | LYS B | 171 | −10.870 | 32.069 | 32.000 | 1.00 | 116.92 |
| 2958 | CB | LYS B | 171 | −12.374 | 32.030 | 31.674 | 1.00 | 111.37 |
| 2959 | CG | LYS B | 171 | −13.021 | 33.402 | 31.721 | 1.00 | 106.02 |
| 2960 | CD | LYS B | 171 | −12.523 | 34.293 | 30.594 | 1.00 | 110.98 |
| 2961 | CE | LYS B | 171 | −13.256 | 35.629 | 30.587 | 1.00 | 109.27 |
| 2962 | NZ | LYS B | 171 | −12.673 | 36.579 | 29.609 | 1.00 | 99.03 |
| 2963 | C | LYS B | 171 | −10.606 | 32.682 | 33.357 | 1.00 | 122.05 |
| 2964 | 0 | LYS B | 171 | −10.455 | 31.952 | 34.327 | 1.00 | 122.45 |
| 2965 | N | ALA B | 172 | −10.550 | 34.004 | 33.492 | 1.00 | 121.93 |
| 2966 | CA | ALA B | 172 | −10.154 | 34.550 | 34.807 | 1.00 | 124.78 |
| 2967 | CB | ALA B | 172 | −8.778 | 35.182 | 34.669 | 1.00 | 110.24 |
| 2968 | C | ALA B | 172 | −11.069 | 35.577 | 35.528 | 1.00 | 131.95 |
| 2969 | O | ALA B | 172 | −11.006 | 36.782 | 35.303 | 1.00 | 136.47 |
| 2970 | N | PRO B | 173 | −11.911 | 35.025 | 36.361 | 1.00 | 138.57 |
| 2971 | CD | PRO B | 173 | −12.775 | 34.002 | 35.754 | 1.00 | 133.76 |
| 2972 | CA | PRO B | 173 | −12.781 | 35.842 | 37.272 | 1.00 | 145.56 |
| 2973 | CB | PRO B | 173 | −13.714 | 34.799 | 37.872 | 1.00 | 143.51 |
| 2974 | CG | PRO B | 173 | −13.935 | 33.894 | 36.705 | 1.00 | 139.83 |
| 2975 | C | PRO B | 173 | −12.008 | 36.690 | 38.273 | 1.00 | 152.09 |
| 2976 | O | PRO B | 173 | −10.896 | 36.332 | 38.614 | 1.00 | 158.41 |
| 2977 | N | ARG B | 174 | −12.559 | 37.804 | 38.749 | 1.00 | 156.36 |
| 2978 | CA | ARG B | 174 | −11.852 | 38.646 | 39.733 | 1.00 | 159.88 |
| 2979 | CB | ARG B | 174 | −10.934 | 39.640 | 39.008 | 1.00 | 160.37 |
| 2980 | CG | ARG B | 174 | −11.660 | 40.518 | 38.000 | 1.00 | 164.23 |
| 2981 | CD | ARG B | 174 | −11.074 | 40.350 | 36.593 | 1.00 | 169.49 |
| 2982 | NE | ARG B | 174 | −12.049 | 40.638 | 35.539 | 1.00 | 176.01 |
| 2983 | CZ | ARG B | 174 | −12.039 | 41.731 | 34.773 | 1.00 | 180.58 |
| 2984 | NH1 | ARG B | 174 | −11.100 | 42.655 | 34.956 | 1.00 | 179.79 |
| 2985 | NH2 | ARG B | 174 | −12.954 | 41.882 | 33.824 | 1.00 | 181.42 |
| 2986 | C | ARG B | 174 | −12.853 | 39.368 | 40.639 | 1.00 | 160.54 |
| 2987 | O | ARG B | 174 | −13.891 | 38.809 | 41.006 | 1.00 | 159.92 |
| 2988 | C1 | NAG B | 221 | 22.996 | 15.148 | 29.775 | 1.00 | 90.21 |
| 2939 | C2 | NAG B | 221 | 23.132 | 14.494 | 28.397 | 1.00 | 106.23 |
| 2990 | N2 | NAG B | 221 | 21.968 | 13.691 | 28.083 | 1.00 | 110.50 |
| 2991 | C7 | NAG B | 221 | 21.087 | 14.132 | 27.187 | 1.00 | 109.71 |
| 2992 | O7 | NAG B | 221 | 21.209 | 15.211 | 26.594 | 1.00 | 98.37 |
| 2993 | C8 | NAG B | 221 | 19.888 | 13.243 | 26.902 | 1.00 | 105.30 |
| 2994 | C3 | NAG B | 221 | 24.395 | 13.644 | 28.376 | 1.00 | 115.44 |
| 2995 | O3 | NAG B | 221 | 24.547 | 13.046 | 27.097 | 1.00 | 116.65 |
| 2996 | C4 | NAG B | 221 | 25.598 | 14.538 | 28.682 | 1.00 | 118.34 |
| 2997 | O4 | NAG B | 221 | 26.785 | 13.757 | 28.731 | 1.00 | 122.24 |
| 2998 | C5 | NAG B | 221 | 25.393 | 15.264 | 30.022 | 1.00 | 113.54 |
| 2999 | O5 | NAG B | 221 | 24.136 | 15.989 | 30.028 | 1.00 | 102.61 |
| 3000 | C6 | NAG B | 221 | 26.494 | 16.278 | 30.269 | 1.00 | 108.41 |
| 3001 | 06 | NAG B | 221 | 26.454 | 16.766 | 31.601 | 1.00 | 119.03 |
| 3002 | C1 | NAG B | 242 | 7.596 | 9.421 | 42.304 | 1.00 | 89.99 |
| 3003 | C2 | NAG B | 242 | 8.393 | 8.151 | 42.040 | 1.00 | 87.43 |
| 3004 | N2 | NAG B | 242 | 9.815 | 8.410 | 42.148 | 1.00 | 86.29 |
| 3005 | C7 | NAG B | 242 | 10.433 | 8.308 | 43.321 | 1.00 | 82.76 |
| 3006 | O7 | NAG B | 242 | 9.852 | 7.998 | 44.367 | 1.00 | 76.22 |
| 3007 | C8 | NAG B | 242 | 11.928 | 8.599 | 43.333 | 1.00 | 77.25 |
| 3008 | C3 | NAG B | 242 | 8.059 | 7.652 | 40.641 | 1.00 | 90.08 |
| 3009 | O3 | NAG B | 242 | 8.693 | 6.400 | 40.436 | 1.00 | 97.55 |
| 3010 | C4 | NAG B | 242 | 6.539 | 7.501 | 40.426 | 1.00 | 91.14 |
| 3011 | O4 | NAG B | 242 | 6.283 | 7.380 | 39.009 | 1.00 | 112.32 |
| 3012 | C5 | NAG B | 242 | 5.757 | 8.728 | 40.956 | 1.00 | 82.98 |
| 3013 | O5 | NAG B | 242 | 6.202 | 9.104 | 42.276 | 1.00 | 91.07 |
| 3014 | C6 | NAG B | 242 | 4.266 | 8.467 | 41.060 | 1.00 | 84.52 |
| 3015 | O6 | NAG B | 242 | 3.514 | 9.640 | 40.779 | 1.00 | 85.63 |
| 3016 | C1 | NAG B | 243 | 6.193 | 6.123 | 38.423 | 1.00 | 105.62 |
| 3017 | C2 | NAG B | 243 | 5.464 | 6.267 | 37.081 | 1.00 | 107.32 |
| 3018 | N2 | NAG B | 243 | 4.122 | 6.776 | 37.289 | 1.00 | 101.51 |
| 3019 | C7 | NAG B | 243 | 3.804 | 7.995 | 36.858 | 1.00 | 90.92 |
| 3020 | O7 | NAG B | 243 | 4.599 | 8.719 | 36.255 | 1.00 | 78.67 |
| 3021 | C8 | NAG B | 243 | 2.389 | 8.483 | 37.120 | 1.00 | 83.10 |
| 3022 | C3 | NAG B | 243 | 5.435 | 4.929 | 36.344 | 1.00 | 112.30 |
| 3023 | 03 | NAG B | 243 | 4.817 | 5.094 | 35.073 | 1.00 | 112.99 |
| 3024 | C4 | NAG B | 243 | 6.875 | 4.455 | 36.166 | 1.00 | 114.52 |
| 3025 | O4 | NAG B | 243 | 6.914 | 3.184 | 35.480 | 1.00 | 130.57 |
| 3026 | C5 | NAG B | 243 | 7.570 | 4.361 | 37.533 | 1.00 | 107.51 |
| 3027 | O5 | NAG B | 243 | 7.529 | 5.646 | 38.195 | 1.00 | 110.47 |
| 3028 | C6 | NAG B | 243 | 9.034 | 3.978 | 37.402 | 1.00 | 100.22 |
| 3029 | O6 | NAG B | 243 | 9.696 | 3.984 | 38.659 | 1.00 | 87.89 |
| 3030 | C1 | MAN B | 244 | 7.657 | 3.203 | 34.307 | 1.00 | 139.64 |
| 3031 | C2 | MAN B | 244 | 6.772 | 2.971 | 33.058 | 1.00 | 140.01 |
| 3032 | 02 | MAN B | 244 | 7.304 | 3.675 | 31.948 | 1.00 | 144.21 |
| 3033 | C3 | MAN B | 244 | 6.590 | 1.496 | 32.679 | 1.00 | 138.91 |
| 3034 | O3 | MAN B | 244 | 6.103 | 1.414 | 31.347 | 1.00 | 144.28 |
| 3035 | C4 | MAN B | 244 | 7.910 | 0.735 | 32.788 | 1.00 | 139.81 |
| 3036 | O4 | MAN B | 244 | 7.708 | −0.643 | 32.517 | 1.00 | 144.34 |
| 3037 | C5 | MAN B | 244 | 8.435 | 0.909 | 34.198 | 1.00 | 140.48 |
| 3038 | OS | MAN B | 244 | 8.779 | 2.294 | 34.406 | 1.00 | 147.09 |
| 3039 | C6 | MAN B | 244 | 9.676 | 0.070 | 34.479 | 1.00 | 136.69 |
| 3040 | O6 | MAN B | 244 | 10.863 | 0.718 | 34.041 | 1.00 | 123.90 |
| 3041 | C1 | NAG B | 335 | 10.916 | 24.720 | 18.959 | 1.00 | 65.94 |
| 3042 | C2 | NAG B | 335 | 12.028 | 24.987 | 17.932 | 1.00 | 79.27 |
| 3043 | N2 | NAG B | 335 | 11.848 | 24.183 | 16.736 | 1.00 | 91.33 |
| 3044 | C7 | NAG B | 335 | 11.340 | 24.727 | 15.629 | 1.00 | 98.55 |
| 3045 | O7 | NAG B | 335 | 10.962 | 25.902 | 15.560 | 1.00 | 81.86 |
| 3046 | C8 | NAG B | 335 | 11.212 | 23.824 | 14.411 | 1.00 | 99.41 |
| 3047 | C3 | NAG B | 335 | 13.413 | 24.705 | 18.548 | 1.00 | 84.72 |
| 3048 | O3 | NAG B | 335 | 14.442 | 25.019 | 17.611 | 1.00 | 91.27 |
| 3049 | C4 | NAG B | 335 | 13.604 | 25.515 | 19.838 | 1.00 | 80.89 |
| 3050 | O4 | NAG B | 335 | 14.831 | 25.090 | 20.488 | 1.00 | 64.07 |
| 3051 | C5 | NAG B | 335 | 12.419 | 25.265 | 20.794 | 1.00 | 64.08 |
| 3052 | OS | NAG B | 335 | 11.144 | 25.538 | 20.131 | 1.00 | 71.94 |
| 3053 | C6 | NAG B | 335 | 12.531 | 26.144 | 22.030 | 1.00 | 64.49 |
| 3054 | O6 | NAG B | 335 | 11.291 | 26.809 | 22.362 | 1.00 | 45.77 |
| 3055 | C1 | NAG B | 336 | 15.929 | 25.939 | 20.563 | 1.00 | 97.76 |
| 3056 | C2 | NAG B | 336 | 16.577 | 25.748 | 21.946 | 1.00 | 97.78 |
| 3057 | N2 | NAG B | 336 | 15.705 | 26.270 | 22.982 | 1.00 | 97.76 |
| 3058 | C7 | NAG B | 336 | 15.077 | 25.437 | 23.810 | 1.00 | 97.93 |
| 3059 | O7 | NAG B | 336 | 15.179 | 24.203 | 23.741 | 1.00 | 97.68 |
| 3060 | C8 | NAG B | 336 | 14.193 | 26.073 | 24.873 | 1.00 | 97.92 |
| 3061 | C3 | NAG B | 336 | 17.943 | 26.425 | 22.064 | 1.00 | 97.90 |
| 3062 | 03 | NAG B | 336 | 18.571 | 25.981 | 23.258 | 1.00 | 98.29 |
| 3063 | C4 | NAG B | 336 | 18.847 | 26.092 | 20.880 | 1.00 | 98.00 |
| 3064 | 04 | NAG B | 336 | 20.012 | 26.915 | 20.922 | 1.00 | 97.87 |
| 3065 | CS | NAG B | 336 | 18.103 | 26.315 | 19.557 | 1.00 | 97.95 |
| 3066 | 05 | NAG B | 336 | 16.862 | 25.561 | 19.525 | 1.00 | 97.83 |
| 3067 | C6 | NAG B | 336 | 18.956 | 25.835 | 18.400 | 1.00 | 97.91 |
| 3068 | 06 | NAG B | 336 | 18.216 | 25.798 | 17.193 | 1.00 | 97.89 |
| 3069 | C1 | FCA B | 337 | 11.537 | 27.883 | 23.223 | 1.00 | 97.62 |
| 3070 | C2 | FCA B | 337 | 10.367 | 28.129 | 24.189 | 1.00 | 97.53 |
| 3071 | C3 | FCA B | 337 | 9.202 | 28.823 | 23.571 | 1.00 | 97.82 |
| 3072 | C4 | FCA B | 337 | 9.595 | 30.213 | 22.961 | 1.00 | 97.76 |
| 3073 | C5 | FCA B | 337 | 10.713 | 29.895 | 21.926 | 1.00 | 97.77 |
| 3074 | C6 | FCA B | 337 | 11.421 | 31.110 | 21.306 | 1.00 | 97.74 |
| 3075 | O2 | FCA B | 337 | 9.934 | 26.823 | 24.727 | 1.00 | 97.69 |
| 3076 | O3 | FCA B | 337 | 8.162 | 29.022 | 24.541 | 1.00 | 97.99 |
| 3077 | O4 | FCA B | 337 | 10.062 | 31.100 | 24.005 | 1.00 | 97.77 |
| 3078 | O5 | FCA B | 337 | 11.775 | 29.137 | 22.508 | 1.00 | 97.61 |
| 3079 | C1 | NAG B | 340 | −0.412 | 38.735 | 24.336 | 1.00 | 122.51 |
| 3080 | C2 | NAG B | 340 | −1.134 | 39.580 | 25.381 | 1.00 | 120.32 |
| 3081 | N2 | NAG B | 340 | −2.513 | 39.812 | 24.998 | 1.00 | 123.12 |
| 3082 | C7 | NAG B | 340 | −3.481 | 39.652 | 25.892 | 1.00 | 119.35 |
| 3083 | O7 | NAG B | 340 | −3.272 | 39.289 | 27.048 | 1.00 | 117.53 |
| 3084 | C8 | NAG B | 340 | −4.900 | 39.928 | 25.422 | 1.00 | 119.54 |
| 3085 | C3 | NAG B | 340 | −0.418 | 40.906 | 25.454 | 1.00 | 119.27 |
| 3086 | O3 | NAG B | 340 | −1.096 | 41.797 | 26.326 | 1.00 | 109.33 |
| 3087 | C4 | NAG B | 340 | 1.035 | 40.774 | 25.885 | 1.00 | 127.45 |
| 3088 | O4 | NAG B | 340 | 1.546 | 42.148 | 25.882 | 1.00 | 140.24 |
| 3089 | C5 | NAG B | 340 | 1.739 | 39.801 | 24.880 | 1.00 | 128.90 |
| 3090 | O5 | NAG B | 340 | 0.965 | 38.548 | 24.746 | 1.00 | 126.48 |
| 3091 | C6 | NAG B | 340 | 3.135 | 39.394 | 25.344 | 1.00 | 127.49 |
| 3092 | O6 | NAG B | 340 | 3.474 | 38.081 | 24.908 | 1.00 | 123.34 |
| 3093 | C1 | NAG B | 341 | 2.837 | 42.676 | 25.784 | 1.00 | 149.17 |
| 3094 | C2 | NAG B | 341 | 3.740 | 42.731 | 27.002 | 1.00 | 145.12 |
| 3095 | N2 | NAG B | 341 | 2.968 | 42.757 | 28.228 | 1.00 | 146.42 |
| 3096 | C7 | NAG B | 341 | 2.704 | 41.612 | 28.847 | 1.00 | 145.07 |
| 3097 | O7 | NAG B | 341 | 3.106 | 40.526 | 28.416 | 1.00 | 132.12 |
| 3098 | C8 | NAG B | 341 | 1.890 | 41.672 | 30.122 | 1.00 | 142.75 |
| 3099 | C3 | NAG B | 341 | 4.552 | 44.017 | 26.806 | 1.00 | 143.92 |
| 3100 | O3 | NAG B | 341 | 5.474 | 44.200 | 27.872 | 1.00 | 142.89 |
| 3101 | C4 | NAG B | 341 | 5.304 | 43.958 | 25.449 | 1.00 | 148.66 |
| 3102 | O4 | NAG B | 341 | 5.954 | 45.201 | 25.210 | 1.00 | 144.18 |
| 3103 | C5 | NAG B | 341 | 4.351 | 43.643 | 24.269 | 1.00 | 155.68 |
| 3104 | O5 | NAG B | 341 | 3.515 | 42.481 | 24.540 | 1.00 | 161.36 |
| 3105 | C6 | NAG B | 341 | 5.092 | 43.354 | 22.983 | 1.00 | 161.88 |
| 3106 | O6 | NAG B | 341 | 4.581 | 44.135 | 21.915 | 1.00 | 165.58 |
| 3107 | C1 | NAG B | 366 | −8.147 | 13.841 | 29.242 | 1.00 | 143.91 |
| 3108 | C2 | NAG B | 366 | −8.310 | 12.851 | 30.401 | 1.00 | 148.60 |
| 3109 | N2 | NAG B | 366 | −7.063 | 12.152 | 30.649 | 1.00 | 155.49 |
| 3110 | C7 | NAG B | 366 | −6.400 | 12.345 | 31.787 | 1.00 | 156.20 |
| 3111 | O7 | NAG B | 366 | −6.791 | 13.114 | 32.670 | 1.00 | 152.95 |
| 3112 | C8 | NAG B | 366 | −5.103 | 11.570 | 31.975 | 1.00 | 155.57 |
| 3113 | C3 | NAG B | 366 | −9.420 | 11.849 | 30.045 | 1.00 | 149.01 |
| 3114 | O3 | NAG B | 366 | −9.658 | 10.967 | 31.133 | 1.00 | 143.79 |
| 3115 | C4 | NAG B | 366 | −10.713 | 12.593 | 29.696 | 1.00 | 151.07 |
| 3116 | O4 | NAG B | 366 | −11.684 | 11.663 | 29.237 | 1.00 | 149.83 |
| 3117 | C5 | NAG B | 366 | −10.447 | 13.654 | 28.611 | 1.00 | 149.31 |
| 3118 | O5 | NAG B | 366 | −9.380 | 14.536 | 29.019 | 1.00 | 146.79 |
| 3119 | C6 | NAG B | 366 | −11.657 | 14.529 | 28.340 | 1.00 | 149.54 |
| 3120 | O6 | NAG B | 366 | −11.370 | 15.902 | 28.578 | 1.00 | 136.69 |

TABLE 8
Atomic coordinates of PhFcεRIα₁₋₁₇₂, Form H1

| ATOM | ATOM | | | | | | | |
|---|---|---|---|---|---|---|---|---|
| NUMBER | TYPE | RESIDUE | # | X | Y | Z | OCC | B |
| 1 | CB | VAL | 1 | 53.051 | 36.792 | 77.715 | 1.00 | 118.55 |
| 2 | CG1 | VAL | 1 | 52.370 | 35.571 | 77.956 | 1.00 | 118.55 |
| 3 | CG2 | VAL | 1 | 53.204 | 37.651 | 79.132 | 1.00 | 118.55 |
| 4 | C | VAL | 1 | 54.467 | 36.682 | 75.485 | 1.00 | 75.78 |
| 5 | O | VAL | 1 | 53.770 | 37.511 | 74.950 | 1.00 | 75.78 |
| 6 | N | VAL | 1 | 55.482 | 37.216 | 77.849 | 1.00 | 75.78 |
| 7 | CA | VAL | 1 | 54.432 | 36.462 | 77.091 | 1.00 | 75.78 |
| 8 | N | PRO | 2 | 55.312 | 35.953 | 74.688 | 1.00 | 130.31 |
| 9 | CD | PRO | 2 | 56.277 | 34.989 | 75.182 | 1.00 | 94.32 |
| 10 | CA | PRO | 2 | 55.477 | 36.015 | 73.216 | 1.00 | 130.31 |
| 11 | CB | PRO | 2 | 56.190 | 34.719 | 72.910 | 1.00 | 94.32 |
| 12 | CG | PRO | 2 | 57.105 | 34.684 | 74.012 | 1.00 | 94.32 |
| 13 | C | PRO | 2 | 54.053 | 36.005 | 72.706 | 1.00 | 130.31 |
| 14 | O | PRO | 2 | 53.189 | 35.367 | 73.293 | 1.00 | 130.31 |
| 15 | N | GLN | 3 | 53.786 | 36.805 | 71.691 | 1.00 | 104.92 |
| 16 | CA | GLN | 3 | 52.463 | 36.918 | 71.130 | 1.00 | 104.92 |
| 17 | CB | GLN | 3 | 52.537 | 37.847 | 69.919 | 1.00 | 99.07 |
| 18 | CG | GLN | 3 | 51.192 | 38.291 | 69.421 | 1.00 | 99.07 |
| 19 | CD | GLN | 3 | 50.249 | 38.740 | 70.542 | 1.00 | 99.07 |
| 20 | OE1 | GLN | 3 | 50.548 | 39.683 | 71.287 | 1.00 | 99.07 |
| 21 | NE2 | GLN | 3 | 49.101 | 38.061 | 70.664 | 1.00 | 99.07 |
| 22 | C | GLN | 3 | 52.005 | 35.499 | 70.762 | 1.00 | 104.92 |
| 23 | O | GLN | 3 | 52.779 | 34.545 | 70.877 | 1.00 | 104.92 |
| 24 | N | LYS | 4 | 50.747 | 35.334 | 70.360 | 1.00 | 70.29 |
| 25 | CA | LYS | 4 | 50.255 | 33.998 | 69.981 | 1.00 | 70.29 |
| 26 | CB | LYS | 4 | 48.731 | 33.942 | 70.002 | 1.00 | 91.56 |
| 27 | CG | LYS | 4 | 47.997 | 34.836 | 69.020 | 1.00 | 91.56 |
| 28 | CD | LYS | 4 | 46.591 | 34.284 | 68.830 | 1.00 | 91.56 |
| 29 | CE | LYS | 4 | 45.661 | 35.309 | 68.214 | 1.00 | 91.56 |
| 30 | NZ | LYS | 4 | 45.296 | 36.401 | 69.165 | 1.00 | 91.56 |
| 31 | C | LYS | 4 | 50.735 | 33.524 | 68.613 | 1.00 | 70.29 |
| 32 | O | LYS | 4 | 50.793 | 34.314 | 67.654 | 1.00 | 70.29 |
| 33 | N | PRO | 5 | 51.056 | 32.216 | 68.503 | 1.00 | 78.97 |
| 34 | CD | PRO | 5 | 51.154 | 31.280 | 69.633 | 1.00 | 108.62 |
| 35 | CA | PRO | 5 | 51.541 | 31.566 | 67.284 | 1.00 | 78.97 |
| 36 | CB | PRO | 5 | 51.808 | 30.127 | 67.739 | 1.00 | 108.62 |
| 37 | CG | PRO | 5 | 52.211 | 30.315 | 69.151 | 1.00 | 108.62 |
| 38 | C | PRO | 5 | 50.601 | 31.638 | 66.091 | 1.00 | 78.97 |
| 39 | O | PRO | 5 | 49.439 | 32.036 | 66.221 | 1.00 | 78.97 |
| 40 | N | LYS | 6 | 51.124 | 31.294 | 64.916 | 1.00 | 88.04 |
| 41 | CA | LYS | 6 | 50.283 | 31.336 | 63.731 | 1.00 | 88.04 |
| 42 | CB | LYS | 6 | 50.440 | 32.681 | 63.010 | 1.00 | 139.11 |
| 43 | CG | LYS | 6 | 49.189 | 33.108 | 62.233 | 1.00 | 139.11 |
| 44 | CD | LYS | 6 | 49.300 | 34.555 | 61.830 | 1.00 | 139.11 |
| 45 | CE | LYS | 6 | 47.998 | 35.105 | 61.293 | 1.00 | 139.11 |
| 46 | NZ | LYS | 6 | 48.130 | 36.569 | 61.033 | 1.00 | 139.11 |
| 47 | C | LYS | 6 | 50.598 | 30.191 | 62.780 | 1.00 | 88.04 |
| 48 | O | LYS | 6 | 51.765 | 29.850 | 62.565 | 1.00 | 88.04 |
| 49 | N | VAL | 7 | 49.539 | 29.590 | 62.235 | 1.00 | 50.50 |
| 50 | CA | VAL | 7 | 49.660 | 28.476 | 61.307 | 1.00 | 50.50 |
| 51 | CB | VAL | 7 | 48.471 | 27.522 | 61.411 | 1.00 | 69.58 |
| 52 | CG1 | VAL | 7 | 48.757 | 26.276 | 60.577 | 1.00 | 69.58 |
| 53 | CG2 | VAL | 7 | 48.212 | 27.169 | 62.858 | 1.00 | 69.58 |
| 54 | C | VAL | 7 | 49.733 | 28.947 | 59.871 | 1.00 | 50.50 |
| 55 | O | VAL | 7 | 48.997 | 29.831 | 59.446 | 1.00 | 50.50 |
| 56 | N | SER | 8 | 50.597 | 28.301 | 59.112 | 1.00 | 106.26 |
| 57 | CA | SER | 8 | 50.788 | 28.646 | 57.723 | 1.00 | 106.26 |
| 58 | CB | SER | 8 | 52.121 | 29.344 | 57.568 | 1.00 | 69.57 |
| 59 | OG | SER | 8 | 53.155 | 28.427 | 57.902 | 1.00 | 69.57 |
| 60 | C | SER | 8 | 50.816 | 27.357 | 56.933 | 1.00 | 106.26 |
| 61 | O | SER | 8 | 51.370 | 26.353 | 57.380 | 1.00 | 106.26 |
| 62 | N | LEU | 9 | 50.221 | 27.381 | 55.754 | 1.00 | 69.51 |
| 63 | CA | LEU | 9 | 50.212 | 26.187 | 54.937 | 1.00 | 69.51 |
| 64 | CB | LEU | 9 | 48.809 | 25.862 | 54.432 | 1.00 | 46.80 |
| 65 | CG | LEU | 9 | 47.549 | 26.374 | 55.111 | 1.00 | 46.80 |
| 66 | CD1 | LEU | 9 | 46.349 | 25.850 | 54.373 | 1.00 | 46.80 |
| 67 | CD2 | LEU | 9 | 47.523 | 25.926 | 56.559 | 1.00 | 46.80 |
| 68 | C | LEU | 9 | 51.111 | 26.317 | 53.718 | 1.00 | 69.51 |
| 69 | O | LEU | 9 | 51.229 | 27.385 | 53.110 | 1.00 | 69.51 |
| 70 | N | ASN | 10 | 51.725 | 25.199 | 53.358 | 1.00 | 65.02 |
| 71 | CA | ASN | 10 | 52.576 | 25.120 | 52.197 | 1.00 | 65.02 |
| 72 | CB | ASN | 10 | 54.050 | 25.035 | 52.622 | 1.00 | 82.93 |
| 73 | CG | ASN | 10 | 54.979 | 24.700 | 51.475 | 1.00 | 82.93 |
| 74 | OD1 | ASN | 10 | 54.991 | 25.378 | 50.451 | 1.00 | 82.93 |
| 75 | ND2 | ASN | 10 | 55.771 | 23.650 | 51.643 | 1.00 | 82.93 |
| 76 | C | ASN | 10 | 52.130 | 23.839 | 51.492 | 1.00 | 65.02 |
| 77 | O | ASN | 10 | 52.431 | 22.728 | 51.953 | 1.00 | 65.02 |
| 78 | N | PRO | 11 | 51.306 | 23.967 | 50.430 | 1.00 | 60.91 |
| 79 | CD | PRO | 11 | 50.772 | 22.738 | 49.827 | 1.00 | 59.89 |
| 80 | CA | PRO | 11 | 50.786 | 25.157 | 49.733 | 1.00 | 60.91 |
| 81 | CB | PRO | 11 | 49.977 | 24.568 | 48.583 | 1.00 | 59.89 |
| 82 | CG | PRO | 11 | 50.471 | 23.159 | 48.449 | 1.00 | 59.89 |
| 83 | C | PRO | 11 | 49.881 | 26.032 | 50.613 | 1.00 | 60.91 |
| 84 | O | PRO | 11 | 49.435 | 25.618 | 51.675 | 1.00 | 60.91 |
| 85 | N | PRO | 12 | 49.570 | 27.245 | 50.142 | 1.00 | 78.15 |
| 86 | CD | PRO | 12 | 50.133 | 27.764 | 48.895 | 1.00 | 41.22 |
| 87 | CA | PRO | 12 | 48.728 | 28.264 | 50.791 | 1.00 | 78.15 |
| 88 | CB | PRO | 12 | 48.984 | 29.528 | 49.967 | 1.00 | 41.22 |
| 89 | CG | PRO | 12 | 50.225 | 29.226 | 49.207 | 1.00 | 41.22 |
| 90 | C | PRO | 12 | 47.264 | 27.890 | 50.704 | 1.00 | 78.15 |
| 91 | O | PRO | 12 | 46.420 | 28.401 | 51.437 | 1.00 | 78.15 |
| 92 | N | TRP | 13 | 46.975 | 27.001 | 49.773 | 1.00 | 63.58 |
| 93 | CA | TRP | 13 | 45.621 | 26.563 | 49.541 | 1.00 | 63.58 |
| 94 | CB | TRP | 13 | 45.619 | 25.679 | 48.308 | 1.00 | 43.81 |
| 95 | CG | TRP | 13 | 46.483 | 26.211 | 47.211 | 1.00 | 43.81 |
| 96 | CD2 | TRP | 13 | 46.444 | 27.519 | 46.634 | 1.00 | 43.81 |
| 97 | CE2 | TRP | 13 | 47.339 | 27.524 | 45.549 | 1.00 | 43.81 |
| 98 | CE3 | TRP | 13 | 45.732 | 28.687 | 46.926 | 1.00 | 43.81 |
| 99 | CD1 | TRP | 13 | 47.385 | 25.507 | 46.480 | 1.00 | 43.81 |
| 100 | NE1 | TRP | 13 | 47.903 | 26.281 | 45.477 | 1.00 | 43.81 |
| 101 | CZ2 | TRP | 13 | 47.543 | 28.650 | 44.746 | 1.00 | 43.81 |
| 102 | CZ3 | TRP | 13 | 45.935 | 29.812 | 46.128 | 1.00 | 43.81 |
| 103 | CH2 | TRP | 13 | 46.838 | 29.780 | 45.046 | 1.00 | 43.81 |
| 104 | C | TRP | 13 | 45.032 | 25.822 | 50.730 | 1.00 | 63.58 |
| 105 | O | TRP | 13 | 45.556 | 24.777 | 51.137 | 1.00 | 63.58 |
| 106 | N | ASN | 14 | 43.947 | 26.377 | 51.280 | 1.00 | 73.67 |
| 107 | CA | ASN | 14 | 43.239 | 25.781 | 52.412 | 1.00 | 73.67 |
| 108 | CB | ASN | 14 | 42.720 | 26.868 | 53.362 | 1.00 | 86.63 |
| 109 | CG | ASN | 14 | 41.638 | 27.726 | 52.745 | 1.00 | 86.63 |
| 110 | OD1 | ASN | 14 | 41.780 | 28.226 | 51.630 | 1.00 | 86.63 |
| 111 | ND2 | ASN | 14 | 40.548 | 27.912 | 53.477 | 1.00 | 86.63 |
| 112 | C | ASN | 14 | 42.090 | 24.940 | 51.861 | 1.00 | 73.67 |
| 113 | O | ASN | 14 | 41.276 | 24.409 | 52.609 | 1.00 | 73.67 |
| 114 | N | ARG | 15 | 42.045 | 24.837 | 50.534 | 1.00 | 50.70 |
| 115 | CA | ARG | 15 | 41.058 | 24.054 | 49.800 | 1.00 | 50.70 |
| 116 | CB | ARG | 15 | 40.174 | 24.947 | 48.943 | 1.00 | 57.52 |
| 117 | CG | ARG | 15 | 39.558 | 26.093 | 49.674 | 1.00 | 57.52 |
| 118 | CD | ARG | 15 | 38.636 | 26.850 | 48.745 | 1.00 | 57.52 |
| 119 | NE | ARG | 15 | 37.242 | 26.435 | 48.833 | 1.00 | 57.52 |
| 120 | CZ | ARG | 15 | 36.364 | 26.620 | 47.853 | 1.00 | 57.52 |
| 121 | NH1 | ARG | 15 | 36.752 | 27.189 | 46.730 | 1.00 | 57.52 |
| 122 | NH2 | ARG | 15 | 35.093 | 26.286 | 47.999 | 1.00 | 57.52 |
| 123 | C | ARG | 15 | 41.851 | 23.139 | 48.858 | 1.00 | 50.70 |
| 124 | O | ARG | 15 | 42.427 | 23.596 | 47.863 | 1.00 | 50.70 |
| 125 | N | ILE | 16 | 41.902 | 21.849 | 49.150 | 1.00 | 58.15 |
| 126 | CA | ILE | 16 | 42.633 | 20.980 | 48.254 | 1.00 | 58.15 |
| 127 | CB | ILE | 16 | 43.956 | 20.548 | 48.866 | 1.00 | 51.72 |
| 128 | CG2 | ILE | 16 | 44.934 | 21.703 | 48.821 | 1.00 | 51.72 |
| 129 | CG1 | ILE | 16 | 43.718 | 20.052 | 50.287 | 1.00 | 51.72 |
| 130 | CD1 | ILE | 16 | 44.968 | 19.557 | 50.978 | 1.00 | 51.72 |
| 131 | C | ILE | 16 | 41.839 | 19.761 | 47.876 | 1.00 | 58.15 |
| 132 | O | ILE | 16 | 40.875 | 19.403 | 48.540 | 1.00 | 58.15 |
| 133 | N | PHE | 17 | 42.260 | 19.157 | 46.776 | 1.00 | 58.19 |
| 134 | CA | PHE | 17 | 41.660 | 17.957 | 46.241 | 1.00 | 58.19 |
| 135 | CB | PHE | 17 | 42.213 | 17.669 | 44.860 | 1.00 | 25.51 |
| 136 | CG | PHE | 17 | 41.536 | 18.414 | 43.776 | 1.00 | 25.51 |
| 137 | CD1 | PHE | 17 | 42.250 | 18.820 | 42.639 | 1.00 | 25.51 |
| 138 | CD2 | PHE | 17 | 40.171 | 18.682 | 43.857 | 1.00 | 25.51 |
| 139 | CE1 | PHE | 17 | 41.608 | 19.487 | 41.592 | 1.00 | 25.51 |
| 140 | CE2 | PHE | 17 | 39.532 | 19.344 | 42.825 | 1.00 | 25.51 |
| 141 | CZ | PHE | 17 | 40.253 | 19.752 | 41.673 | 1.00 | 25.51 |
| 142 | C | PHE | 17 | 42.019 | 16.789 | 47.117 | 1.00 | 58.19 |
| 143 | O | PHE | 17 | 43.030 | 16.815 | 47.836 | 1.00 | 58.19 |
| 144 | N | LYS | 18 | 41.202 | 15.749 | 47.024 | 1.00 | 64.21 |
| 145 | CA | LYS | 18 | 41.421 | 14.528 | 47.770 | 1.00 | 64.21 |
| 146 | CB | LYS | 18 | 40.266 | 13.572 | 47.476 | 1.00 | 120.30 |
| 147 | CG | LYS | 18 | 40.180 | 12.351 | 48.352 | 1.00 | 120.30 |
| 148 | CD | LYS | 18 | 38.856 | 11.639 | 48.078 | 1.00 | 120.30 |
| 149 | CE | LYS | 18 | 38.656 | 10.437 | 48.983 | 1.00 | 120.30 |
| 150 | NZ | LYS | 18 | 39.547 | 9.324 | 48.594 | 1.00 | 120.30 |
| 151 | C | LYS | 18 | 42.740 | 13.978 | 47.237 | 1.00 | 64.21 |
| 152 | O | LYS | 18 | 42.970 | 13.995 | 46.027 | 1.00 | 64.21 |
| 153 | N | GLY | 19 | 43.619 | 13.536 | 48.134 | 1.00 | 59.20 |
| 154 | CA | GLY | 19 | 44.893 | 12.963 | 47.706 | 1.00 | 59.20 |
| 155 | C | GLY | 19 | 46.112 | 13.851 | 47.506 | 1.00 | 59.20 |
| 156 | O | GLY | 19 | 47.199 | 13.352 | 47.174 | 1.00 | 59.20 |
| 157 | N | GLU | 20 | 45.938 | 15.156 | 47.686 | 1.00 | 96.38 |
| 158 | CA | GLU | 20 | 47.046 | 16.087 | 47.539 | 1.00 | 96.38 |
| 159 | CB | GLU | 20 | 46.547 | 17.429 | 46.993 | 1.00 | 62.66 |
| 160 | CG | GLU | 20 | 45.820 | 17.303 | 45.661 | 1.00 | 62.66 |
| 161 | CD | GLU | 20 | 45.468 | 18.648 | 45.025 | 1.00 | 62.66 |
| 162 | OE1 | GLU | 20 | 44.899 | 19.525 | 45.726 | 1.00 | 62.66 |
| 163 | OE2 | GLU | 20 | 45.754 | 18.811 | 43.815 | 1.00 | 62.66 |
| 164 | C | GLU | 20 | 47.666 | 16.261 | 48.917 | 1.00 | 96.38 |
| 165 | O | GLU | 20 | 47.055 | 15.899 | 49.922 | 1.00 | 96.38 |
| 166 | N | ASN | 21 | 48.886 | 16.783 | 48.966 | 1.00 | 82.14 |
| 167 | CA | ASN | 21 | 49.550 | 16.992 | 50.245 | 1.00 | 82.14 |
| 168 | CB | ASN | 21 | 50.929 | 16.375 | 50.212 | 1.00 | 83.98 |
| 169 | CG | ASN | 21 | 50.907 | 15.025 | 49.599 | 1.00 | 83.98 |
| 170 | OD1 | ASN | 21 | 50.088 | 14.203 | 49.969 | 1.00 | 83.98 |
| 171 | ND2 | ASN | 21 | 51.791 | 14.775 | 48.651 | 1.00 | 83.98 |
| 172 | C | ASN | 21 | 49.661 | 18.458 | 50.598 | 1.00 | 82.14 |
| 173 | O | ASN | 21 | 49.706 | 19.317 | 49.727 | 1.00 | 82.14 |
| 174 | N | VAL | 22 | 49.715 | 18.739 | 51.889 | 1.00 | 82.30 |
| 175 | CA | VAL | 22 | 49.809 | 20.109 | 52.347 | 1.00 | 82.30 |
| 176 | CB | VAL | 22 | 48.407 | 20.695 | 52.489 | 1.00 | 52.62 |
| 177 | CG1 | VAL | 22 | 47.687 | 19.993 | 53.637 | 1.00 | 52.62 |
| 178 | CG2 | VAL | 22 | 48.473 | 22.229 | 52.679 | 1.00 | 52.62 |
| 179 | C | VAL | 22 | 50.502 | 20.090 | 53.698 | 1.00 | 82.30 |
| 180 | O | VAL | 22 | 50.248 | 19.192 | 54.503 | 1.00 | 82.30 |
| 181 | N | THR | 23 | 51.376 | 21.063 | 53.954 | 1.00 | 73.57 |
| 182 | CA | THR | 23 | 52.083 | 21.109 | 55.231 | 1.00 | 73.57 |
| 183 | CB | THR | 23 | 53.598 | 21.205 | 55.035 | 1.00 | 78.82 |
| 184 | OG1 | THR | 23 | 54.021 | 20.223 | 54.076 | 1.00 | 78.82 |
| 185 | CG2 | THR | 23 | 54.309 | 20.974 | 56.380 | 1.00 | 78.82 |
| 186 | C | THR | 23 | 51.658 | 22.281 | 56.097 | 1.00 | 73.57 |
| 187 | O | THR | 23 | 51.549 | 23.407 | 55.618 | 1.00 | 73.57 |
| 188 | N | LEU | 24 | 51.417 | 22.007 | 57.374 | 1.00 | 80.82 |
| 189 | CA | LEU | 24 | 51.026 | 23.040 | 58.314 | 1.00 | 80.82 |
| 190 | CB | LEU | 24 | 49.818 | 22.605 | 59.138 | 1.00 | 72.07 |
| 191 | CG | LEU | 24 | 48.578 | 22.217 | 58.346 | 1.00 | 72.07 |
| 192 | CD1 | LEU | 24 | 47.353 | 22.285 | 59.242 | 1.00 | 72.07 |
| 193 | CD2 | LEU | 24 | 48.412 | 23.157 | 57.182 | 1.00 | 72.07 |
| 194 | C | LEU | 24 | 52.188 | 23.322 | 59.239 | 1.00 | 80.82 |
| 195 | O | LEU | 24 | 52.771 | 22.420 | 59.825 | 1.00 | 80.82 |
| 196 | N | THR | 25 | 52.527 | 24.591 | 59.367 | 1.00 | 57.44 |
| 197 | CA | THR | 25 | 53.622 | 24.995 | 60.228 | 1.00 | 57.44 |
| 198 | CB | THR | 25 | 54.748 | 25.593 | 59.431 | 1.00 | 58.08 |
| 199 | OG1 | THR | 25 | 55.299 | 24.593 | 58.568 | 1.00 | 58.08 |
| 200 | CG2 | THR | 25 | 55.812 | 26.103 | 60.372 | 1.00 | 58.08 |
| 201 | C | THR | 25 | 53.197 | 26.039 | 61.238 | 1.00 | 57.44 |
| 202 | O | THR | 25 | 52.524 | 27.007 | 60.891 | 1.00 | 57.44 |
| 203 | N | CYS | 26 | 53.589 | 25.833 | 62.489 | 1.00 | 94.33 |
| 204 | CA | CYS | 26 | 53.276 | 26.780 | 63.551 | 1.00 | 94.33 |
| 205 | C | CYS | 26 | 54.464 | 27.725 | 63.557 | 1.00 | 94.33 |
| 206 | O | CYS | 26 | 55.604 | 27.267 | 63.459 | 1.00 | 94.33 |
| 207 | CB | CYS | 26 | 53.206 | 26.060 | 64.890 | 1.00 | 89.97 |
| 208 | SG | CYS | 26 | 52.191 | 26.860 | 66.176 | 1.00 | 89.97 |
| 209 | N | ASN | 27 | 54.229 | 29.031 | 63.651 | 1.00 | 88.38 |
| 210 | CA | ASN | 27 | 55.363 | 29.941 | 63.665 | 1.00 | 88.38 |
| 211 | CB | ASN | 27 | 55.584 | 30.555 | 62.273 | 1.00 | 163.21 |
| 212 | CG | ASN | 27 | 56.225 | 29.574 | 61.290 | 1.00 | 163.21 |
| 213 | OD1 | ASN | 27 | 57.082 | 28.768 | 61.667 | 1.00 | 163.21 |
| 214 | ND2 | ASN | 27 | 55.825 | 29.654 | 60.020 | 1.00 | 163.21 |
| 215 | C | ASN | 27 | 55.336 | 31.039 | 64.719 | 1.00 | 88.38 |
| 216 | O | ASN | 27 | 54.563 | 31.989 | 64.641 | 1.00 | 88.38 |
| 217 | N | GLY | 28 | 56.164 | 30.862 | 65.736 | 1.00 | 157.55 |
| 218 | CA | GLY | 28 | 56.306 | 31.863 | 66.769 | 1.00 | 157.55 |
| 219 | C | GLY | 28 | 57.695 | 32.269 | 66.353 | 1.00 | 157.55 |
| 220 | O | GLY | 28 | 58.644 | 31.591 | 66.702 | 1.00 | 157.55 |
| 221 | N | ASN | 29 | 57.813 | 33.337 | 65.572 | 1.00 | 155.07 |
| 222 | CA | ASN | 29 | 59.110 | 33.779 | 65.057 | 1.00 | 155.07 |
| 223 | CB | ASN | 29 | 59.054 | 35.252 | 64.655 | 1.00 | 162.85 |
| 224 | CG | ASN | 29 | 57.897 | 35.564 | 63.723 | 1.00 | 162.85 |
| 225 | OD1 | ASN | 29 | 57.735 | 34.939 | 62.672 | 1.00 | 162.85 |
| 226 | ND2 | ASN | 29 | 57.088 | 36.545 | 64.103 | 1.00 | 162.85 |
| 227 | C | ASN | 29 | 60.312 | 33.547 | 65.963 | 1.00 | 155.07 |
| 228 | O | ASN | 29 | 61.386 | 33.183 | 65.471 | 1.00 | 155.07 |
| 229 | N | ASN | 30 | 60.152 | 33.787 | 67.269 | 1.00 | 156.06 |
| 230 | CA | ASN | 30 | 61.241 | 33.545 | 68.220 | 1.00 | 156.06 |
| 231 | CB | ASN | 30 | 60.696 | 33.490 | 69.653 | 1.00 | 177.59 |
| 232 | CG | ASN | 30 | 60.720 | 34.850 | 70.334 | 1.00 | 177.59 |
| 233 | OD1 | ASN | 30 | 61.733 | 35.550 | 70.290 | 1.00 | 177.59 |
| 234 | ND2 | ASN | 30 | 59.613 | 35.227 | 70.970 | 1.00 | 177.59 |
| 235 | C | ASN | 30 | 61.697 | 32.182 | 67.729 | 1.00 | 156.06 |
| 236 | O | ASN | 30 | 62.886 | 31.932 | 67.477 | 1.00 | 156.06 |
| 237 | N | PHE | 31 | 60.702 | 31.316 | 67.577 | 1.00 | 152.23 |
| 238 | CA | PHE | 31 | 60.896 | 30.010 | 66.990 | 1.00 | 152.23 |
| 239 | CB | PHE | 31 | 61.081 | 30.267 | 65.497 | 1.00 | 158.06 |
| 240 | CG | PHE | 31 | 60.650 | 29.154 | 64.612 | 1.00 | 158.06 |
| 241 | CD1 | PHE | 31 | 59.315 | 28.746 | 64.550 | 1.00 | 158.06 |
| 242 | CD2 | PHE | 31 | 61.573 | 28.561 | 63.769 | 1.00 | 158.06 |
| 243 | CE1 | PHE | 31 | 58.924 | 27.755 | 63.653 | 1.00 | 158.06 |
| 244 | CE2 | PHE | 31 | 61.197 | 27.579 | 62.877 | 1.00 | 158.06 |
| 245 | CZ | PHE | 31 | 59.869 | 27.173 | 62.810 | 1.00 | 158.06 |
| 246 | C | PHE | 31 | 62.099 | 29.281 | 67.576 | 1.00 | 152.23 |
| 247 | O | PHE | 31 | 62.836 | 28.610 | 66.857 | 1.00 | 152.23 |
| 248 | N | PHE | 32 | 62.304 | 29.414 | 68.880 | 1.00 | 166.63 |
| 249 | CA | PHE | 32 | 63.432 | 28.752 | 69.510 | 1.00 | 166.63 |
| 250 | CB | PHE | 32 | 63.802 | 29.479 | 70.792 | 1.00 | 178.95 |
| 251 | CG | PHE | 32 | 64.123 | 30.923 | 70.567 | 1.00 | 178.95 |
| 252 | CD1 | PHE | 32 | 63.361 | 31.922 | 71.164 | 1.00 | 178.95 |
| 253 | CD2 | PHE | 32 | 65.174 | 31.288 | 69.723 | 1.00 | 178.95 |
| 254 | CE1 | PHE | 32 | 63.631 | 33.271 | 70.914 | 1.00 | 178.95 |
| 255 | CE2 | PHE | 32 | 65.452 | 32.634 | 69.466 | 1.00 | 178.95 |
| 256 | CZ | PHE | 32 | 64.681 | 33.629 | 70.068 | 1.00 | 178.95 |
| 257 | C | PHE | 32 | 63.116 | 27.288 | 69.760 | 1.00 | 166.63 |
| 258 | O | PHE | 32 | 63.884 | 26.567 | 70.399 | 1.00 | 166.63 |
| 259 | N | GLU | 33 | 61.968 | 26.864 | 69.239 | 1.00 | 156.85 |
| 260 | CA | GLU | 33 | 61.527 | 25.475 | 69.315 | 1.00 | 156.85 |
| 261 | CB | GLU | 33 | 62.558 | 24.582 | 68.620 | 1.00 | 168.05 |
| 262 | CG | GLU | 33 | 63.153 | 25.157 | 67.330 | 1.00 | 168.05 |
| 263 | CD | GLU | 33 | 64.130 | 24.188 | 66.704 | 1.00 | 168.05 |
| 264 | OE1 | GLU | 33 | 64.854 | 23.526 | 67.477 | 1.00 | 168.05 |
| 265 | OE2 | GLU | 33 | 64.175 | 24.084 | 65.461 | 1.00 | 168.05 |
| 266 | C | GLU | 33 | 61.233 | 24.913 | 70.708 | 1.00 | 156.85 |
| 267 | O | GLU | 33 | 60.369 | 24.046 | 70.851 | 1.00 | 156.85 |
| 268 | N | VAL | 34 | 61.963 | 25.376 | 71.719 | 1.00 | 106.92 |
| 269 | CA | VAL | 34 | 61.748 | 24.926 | 73.095 | 1.00 | 106.92 |
| 270 | CB | VAL | 34 | 60.953 | 25.998 | 73.882 | 1.00 | 129.93 |
| 271 | CG1 | VAL | 34 | 60.864 | 25.626 | 75.361 | 1.00 | 129.93 |
| 272 | CG2 | VAL | 34 | 61.607 | 27.359 | 73.695 | 1.00 | 129.93 |
| 273 | C | VAL | 34 | 61.011 | 23.573 | 73.211 | 1.00 | 106.92 |
| 274 | O | VAL | 34 | 59.825 | 23.521 | 73.562 | 1.00 | 106.92 |
| 275 | N | SER | 35 | 61.712 | 22.490 | 72.883 | 1.00 | 178.50 |
| 276 | CA | SER | 35 | 61.170 | 21.135 | 72.978 | 1.00 | 178.50 |
| 277 | CB | SER | 35 | 60.683 | 20.879 | 74.409 | 1.00 | 177.04 |
| 278 | OG | SER | 35 | 61.781 | 20.767 | 75.302 | 1.00 | 177.04 |
| 279 | C | SER | 35 | 60.102 | 20.645 | 71.988 | 1.00 | 178.50 |
| 280 | O | SER | 35 | 60.449 | 20.069 | 70.956 | 1.00 | 178.50 |
| 281 | N | SER | 36 | 58.816 | 20.858 | 72.286 | 1.00 | 97.16 |
| 282 | CA | SER | 36 | 57.742 | 20.347 | 71.411 | 1.00 | 97.16 |
| 283 | CB | SER | 36 | 57.175 | 19.038 | 71.993 | 1.00 | 145.01 |
| 284 | OG | SER | 36 | 58.160 | 18.018 | 72.059 | 1.00 | 145.01 |
| 285 | C | SER | 36 | 56.560 | 21.259 | 71.061 | 1.00 | 97.16 |
| 286 | O | SER | 36 | 56.405 | 22.357 | 71.604 | 1.00 | 97.16 |
| 287 | N | THR | 37 | 55.725 | 20.750 | 70.149 | 1.00 | 111.12 |
| 288 | CA | THR | 37 | 54.530 | 21.433 | 69.638 | 1.00 | 111.12 |
| 289 | CB | THR | 37 | 54.686 | 21.780 | 68.144 | 1.00 | 142.34 |
| 290 | OG1 | THR | 37 | 55.863 | 22.572 | 67.954 | 1.00 | 142.34 |
| 291 | CG2 | THR | 37 | 53.465 | 22.536 | 67.638 | 1.00 | 142.34 |
| 292 | C | THR | 37 | 53.273 | 20.565 | 69.746 | 1.00 | 111.12 |
| 293 | O | THR | 37 | 53.323 | 19.357 | 69.509 | 1.00 | 111.12 |
| 294 | N | LYS | 38 | 52.147 | 21.193 | 70.075 | 1.00 | 89.74 |
| 295 | CA | LYS | 38 | 50.879 | 20.480 | 70.195 | 1.00 | 89.74 |
| 296 | CB | LYS | 38 | 50.253 | 20.739 | 71.559 | 1.00 | 101.72 |
| 297 | CG | LYS | 38 | 50.833 | 19.880 | 72.648 | 1.00 | 101.72 |
| 298 | CD | LYS | 38 | 50.223 | 20.211 | 73.991 | 1.00 | 101.72 |
| 299 | CE | LYS | 38 | 50.893 | 19.410 | 75.110 | 1.00 | 101.72 |
| 300 | NZ | LYS | 38 | 50.571 | 19.965 | 76.465 | 1.00 | 101.72 |
| 301 | C | LYS | 38 | 49.889 | 20.878 | 69.112 | 1.00 | 89.74 |
| 302 | O | LYS | 38 | 49.478 | 22.036 | 69.039 | 1.00 | 89.74 |
| 303 | N | TRP | 39 | 49.501 | 19.911 | 68.280 | 1.00 | 92.70 |
| 304 | CA | TRP | 39 | 48.549 | 20.162 | 67.196 | 1.00 | 92.70 |
| 305 | CB | TRP | 39 | 48.948 | 19.417 | 65.933 | 1.00 | 89.95 |
| 306 | CG | TRP | 39 | 50.148 | 19.958 | 65.279 | 1.00 | 89.95 |
| 307 | CD2 | TRP | 39 | 50.188 | 21.016 | 64.329 | 1.00 | 89.95 |
| 308 | CE2 | TRP | 39 | 51.536 | 21.192 | 63.941 | 1.00 | 89.95 |
| 309 | CE3 | TRP | 39 | 49.211 | 21.846 | 63.759 | 1.00 | 89.95 |
| 310 | CD1 | TRP | 39 | 51.438 | 19.535 | 65.441 | 1.00 | 89.95 |
| 311 | NE1 | TRP | 39 | 52.282 | 20.269 | 64.639 | 1.00 | 89.95 |
| 312 | CZ2 | TRP | 39 | 51.938 | 22.145 | 63.017 | 1.00 | 89.95 |
| 313 | CZ3 | TRP | 39 | 49.603 | 22.799 | 62.839 | 1.00 | 89.95 |
| 314 | CH2 | TRP | 39 | 50.958 | 22.943 | 62.475 | 1.00 | 89.95 |
| 315 | C | TRP | 39 | 47.139 | 19.743 | 67.541 | 1.00 | 92.70 |
| 316 | O | TRP | 39 | 46.926 | 18.717 | 68.177 | 1.00 | 92.70 |
| 317 | N | PHE | 40 | 46.167 | 20.522 | 67.096 | 1.00 | 74.21 |
| 318 | CA | PHE | 40 | 44.782 | 20.188 | 67.382 | 1.00 | 74.21 |
| 319 | CB | PHE | 40 | 44.177 | 21.186 | 68.365 | 1.00 | 91.61 |
| 320 | CG | PHE | 40 | 44.868 | 21.229 | 69.698 | 1.00 | 91.61 |
| 321 | CD1 | PHE | 40 | 46.143 | 21.793 | 69.829 | 1.00 | 91.61 |
| 322 | CD2 | PHE | 40 | 44.231 | 20.730 | 70.833 | 1.00 | 91.61 |
| 323 | CE1 | PHE | 40 | 46.761 | 21.876 | 71.069 | 1.00 | 91.61 |
| 324 | CE2 | PHE | 40 | 44.839 | 20.807 | 72.078 | 1.00 | 91.61 |
| 325 | CZ | PHE | 40 | 46.110 | 21.379 | 72.196 | 1.00 | 91.61 |
| 326 | C | PHE | 40 | 43.946 | 20.149 | 66.121 | 1.00 | 74.21 |
| 327 | O | PHE | 40 | 43.709 | 21.176 | 65.474 | 1.00 | 74.21 |
| 328 | N | HIS | 41 | 43.506 | 18.948 | 65.774 | 1.00 | 72.61 |
| 329 | CA | HIS | 41 | 42.691 | 18.750 | 64.593 | 1.00 | 72.61 |
| 330 | CB | HIS | 41 | 43.150 | 17.481 | 63.880 | 1.00 | 96.01 |
| 331 | CG | HIS | 41 | 42.346 | 17.151 | 62.661 | 1.00 | 96.01 |
| 332 | CD2 | HIS | 41 | 42.300 | 16.023 | 61.911 | 1.00 | 96.01 |
| 333 | ND1 | HIS | 41 | 41.489 | 18.048 | 62.073 | 1.00 | 96.01 |
| 334 | CE1 | HIS | 41 | 40.942 | 17.489 | 61.005 | 1.00 | 96.01 |
| 335 | NE2 | HIS | 41 | 41.419 | 16.264 | 60.887 | 1.00 | 96.01 |
| 336 | C | HIS | 41 | 41.223 | 18.646 | 64.999 | 1.00 | 72.61 |
| 337 | O | HIS | 41 | 40.831 | 17.703 | 65.696 | 1.00 | 72.61 |
| 338 | N | ASN | 42 | 40.419 | 19.616 | 64.563 | 1.00 | 65.96 |
| 339 | CA | ASN | 42 | 38.998 | 19.638 | 64.896 | 1.00 | 65.96 |
| 340 | CB | ASN | 42 | 38.304 | 18.373 | 64.347 | 1.00 | 107.42 |
| 341 | CG | ASN | 42 | 37.832 | 18.533 | 62.897 | 1.00 | 107.42 |
| 342 | OD1 | ASN | 42 | 38.115 | 19.550 | 62.262 | 1.00 | 107.42 |
| 343 | ND2 | ASN | 42 | 37.110 | 17.532 | 62.380 | 1.00 | 107.42 |
| 344 | C | ASN | 42 | 38.869 | 19.713 | 66.422 | 1.00 | 65.96 |
| 345 | O | ASN | 42 | 37.863 | 19.297 | 67.008 | 1.00 | 65.96 |
| 346 | N | GLY | 43 | 39.916 | 20.229 | 67.058 | 1.00 | 142.69 |
| 347 | CA | GLY | 43 | 39.930 | 20.362 | 68.505 | 1.00 | 142.69 |
| 348 | C | GLY | 43 | 40.418 | 19.152 | 69.292 | 1.00 | 142.69 |
| 349 | O | GLY | 43 | 40.304 | 19.125 | 70.508 | 1.00 | 142.69 |
| 350 | N | SER | 44 | 40.972 | 18.149 | 68.621 | 1.00 | 82.57 |
| 351 | CA | SER | 44 | 41.448 | 16.960 | 69.318 | 1.00 | 82.57 |
| 352 | CB | SER | 44 | 40.876 | 15.716 | 68.658 | 1.00 | 65.56 |
| 353 | OG | SER | 44 | 41.042 | 14.585 | 69.496 | 1.00 | 65.56 |
| 354 | C | SER | 44 | 42.966 | 16.877 | 69.294 | 1.00 | 82.57 |
| 355 | O | SER | 44 | 43.558 | 16.765 | 68.218 | 1.00 | 82.57 |
| 356 | N | LEU | 45 | 43.601 | 16.910 | 70.464 | 1.00 | 83.34 |
| 357 | CA | LEU | 45 | 45.056 | 16.846 | 70.490 | 1.00 | 83.34 |
| 358 | CB | LEU | 45 | 45.586 | 16.632 | 71.930 | 1.00 | 79.17 |
| 359 | CG | LEU | 45 | 47.121 | 16.657 | 72.177 | 1.00 | 79.17 |
| 360 | CD1 | LEU | 45 | 47.758 | 17.921 | 71.590 | 1.00 | 79.17 |
| 361 | CD2 | LEU | 45 | 47.403 | 16.581 | 73.681 | 1.00 | 79.17 |
| 362 | C | LEU | 45 | 45.537 | 15.736 | 69.542 | 1.00 | 83.34 |
| 363 | O | LEU | 45 | 45.074 | 14.588 | 69.585 | 1.00 | 83.34 |
| 364 | N | SER | 46 | 46.447 | 16.125 | 68.658 | 1.00 | 133.67 |
| 365 | CA | SER | 46 | 47.037 | 15.236 | 67.671 | 1.00 | 133.67 |
| 366 | CB | SER | 46 | 47.551 | 16.059 | 66.487 | 1.00 | 75.61 |
| 367 | OG | SER | 46 | 48.435 | 15.315 | 65.662 | 1.00 | 75.61 |
| 368 | C | SER | 46 | 48.196 | 14.486 | 68.299 | 1.00 | 133.67 |
| 369 | O | SER | 46 | 48.864 | 15.000 | 69.195 | 1.00 | 133.67 |
| 370 | N | GLU | 47 | 48.431 | 13.268 | 67.827 | 1.00 | 113.73 |
| 371 | CA | GLU | 47 | 49.545 | 12.470 | 68.329 | 1.00 | 113.73 |
| 372 | CB | GLU | 47 | 49.354 | 10.985 | 67.982 | 1.00 | 165.00 |
| 373 | CG | GLU | 47 | 48.217 | 10.318 | 68.775 | 1.00 | 165.00 |
| 374 | CD | GLU | 47 | 48.093 | 8.817 | 68.530 | 1.00 | 165.00 |
| 375 | OE1 | GLU | 47 | 47.813 | 8.412 | 67.380 | 1.00 | 165.00 |
| 376 | OE2 | GLU | 47 | 48.270 | 8.039 | 69.494 | 1.00 | 165.00 |
| 377 | C | GLU | 47 | 50.821 | 13.023 | 67.695 | 1.00 | 113.73 |
| 378 | O | GLU | 47 | 51.921 | 12.506 | 67.908 | 1.00 | 113.73 |
| 379 | N | GLU | 48 | 50.650 | 14.089 | 66.915 | 1.00 | 132.93 |
| 380 | CA | GLU | 48 | 51.755 | 14.763 | 66.249 | 1.00 | 132.93 |
| 381 | CB | GLU | 48 | 51.264 | 15.424 | 64.966 | 1.00 | 192.46 |
| 382 | CG | GLU | 48 | 52.348 | 16.113 | 64.173 | 1.00 | 192.46 |
| 383 | CD | GLU | 48 | 52.450 | 15.579 | 62.762 | 1.00 | 192.46 |
| 384 | OE1 | GLU | 48 | 51.665 | 14.672 | 62.406 | 1.00 | 192.46 |
| 385 | OE2 | GLU | 48 | 53.316 | 16.062 | 62.005 | 1.00 | 192.46 |
| 386 | C | GLU | 48 | 52.280 | 15.826 | 67.207 | 1.00 | 132.93 |
| 387 | O | GLU | 48 | 51.506 | 16.635 | 67.730 | 1.00 | 132.93 |
| 388 | N | THR | 49 | 53.591 | 15.827 | 67.436 | 1.00 | 120.56 |
| 389 | CA | THR | 49 | 54.193 | 16.789 | 68.350 | 1.00 | 120.56 |
| 390 | CB | THR | 49 | 54.823 | 16.075 | 69.569 | 1.00 | 154.93 |
| 391 | OG1 | THR | 49 | 55.638 | 14.984 | 69.123 | 1.00 | 154.93 |
| 392 | CG2 | THR | 49 | 53.740 | 15.552 | 70.504 | 1.00 | 154.93 |
| 393 | C | THR | 49 | 55.248 | 17.684 | 67.713 | 1.00 | 120.56 |
| 394 | O | THR | 49 | 55.697 | 18.652 | 68.323 | 1.00 | 120.56 |
| 395 | N | ASN | 50 | 55.639 | 17.368 | 66.484 | 1.00 | 120.79 |
| 396 | CA | ASN | 50 | 56.646 | 18.154 | 65.779 | 1.00 | 120.79 |
| 397 | CB | ASN | 50 | 57.190 | 17.349 | 64.596 | 1.00 | 183.19 |
| 398 | CG | ASN | 50 | 57.793 | 16.024 | 65.035 | 1.00 | 183.19 |
| 399 | OD1 | ASN | 50 | 58.218 | 15.900 | 66.182 | 1.00 | 183.19 |
| 400 | ND2 | ASN | 50 | 57.865 | 15.054 | 64.124 | 1.00 | 183.19 |
| 401 | C | ASN | 50 | 56.070 | 19.485 | 65.312 | 1.00 | 120.79 |
| 402 | O | ASN | 50 | 54.854 | 19.616 | 65.160 | 1.00 | 120.79 |
| 403 | N | SER | 51 | 56.944 | 20.466 | 65.084 | 1.00 | 104.18 |
| 404 | CA | SER | 51 | 56.522 | 21.811 | 64.657 | 1.00 | 104.18 |
| 405 | CB | SER | 51 | 57.735 | 22.751 | 64.577 | 1.00 | 100.13 |
| 406 | OG | SER | 51 | 58.559 | 22.431 | 63.468 | 1.00 | 100.13 |
| 407 | C | SER | 51 | 55.753 | 21.880 | 63.329 | 1.00 | 104.18 |
| 408 | O | SER | 51 | 55.086 | 22.879 | 63.053 | 1.00 | 104.18 |
| 409 | N | SER | 52 | 55.858 | 20.835 | 62.507 | 1.00 | 108.20 |
| 410 | CA | SER | 52 | 55.154 | 20.803 | 61.223 | 1.00 | 108.20 |
| 411 | CB | SER | 52 | 56.131 | 20.860 | 60.041 | 1.00 | 83.34 |
| 412 | OG | SER | 52 | 56.705 | 22.151 | 59.913 | 1.00 | 83.34 |
| 413 | C | SER | 52 | 54.291 | 19.566 | 61.083 | 1.00 | 108.20 |
| 414 | O | SER | 52 | 54.750 | 18.445 | 61.284 | 1.00 | 108.20 |
| 415 | N | LEU | 53 | 53.038 | 19.790 | 60.720 | 1.00 | 62.72 |
| 416 | CA | LEU | 53 | 52.057 | 18.730 | 60.536 | 1.00 | 62.72 |
| 417 | CB | LEU | 53 | 50.730 | 19.214 | 61.130 | 1.00 | 43.93 |
| 418 | CG | LEU | 53 | 49.456 | 18.379 | 61.050 | 1.00 | 43.93 |
| 419 | CD1 | LEU | 53 | 49.665 | 17.079 | 61.786 | 1.00 | 43.93 |
| 420 | CD2 | LEU | 53 | 48.305 | 19.148 | 61.680 | 1.00 | 43.93 |
| 421 | C | LEU | 53 | 51.912 | 18.398 | 59.035 | 1.00 | 62.72 |
| 422 | O | LEU | 53 | 51.470 | 19.226 | 58.234 | 1.00 | 62.72 |
| 423 | N | ASN | 54 | 52.294 | 17.192 | 58.642 | 1.00 | 92.98 |
| 424 | CA | ASN | 54 | 52.183 | 16.832 | 57.236 | 1.00 | 92.98 |
| 425 | CB | ASN | 54 | 53.404 | 16.036 | 56.796 | 1.00 | 86.69 |
| 426 | CG | ASN | 54 | 54.670 | 16.844 | 56.872 | 1.00 | 86.69 |
| 427 | OD1 | ASN | 54 | 54.805 | 17.866 | 56.186 | 1.00 | 86.69 |
| 428 | ND2 | ASN | 54 | 55.608 | 16.406 | 57.717 | 1.00 | 86.69 |
| 429 | C | ASN | 54 | 50.941 | 16.029 | 56.929 | 1.00 | 92.98 |
| 430 | O | ASN | 54 | 50.701 | 14.991 | 57.529 | 1.00 | 92.98 |
| 431 | N | ILE | 55 | 50.144 | 16.523 | 55.995 | 1.00 | 45.24 |
| 432 | CA | ILE | 55 | 48.935 | 15.823 | 55.582 | 1.00 | 45.24 |
| 433 | CB | ILE | 55 | 47.716 | 16.773 | 55.491 | 1.00 | 31.24 |
| 434 | CG2 | ILE | 55 | 46.623 | 16.157 | 54.647 | 1.00 | 31.24 |
| 435 | CG1 | ILE | 55 | 47.183 | 17.072 | 56.884 | 1.00 | 31.24 |
| 436 | CD1 | ILE | 55 | 45.969 | 17.961 | 56.867 | 1.00 | 31.24 |
| 437 | C | ILE | 55 | 49.224 | 15.251 | 54.200 | 1.00 | 45.24 |
| 438 | O | ILE | 55 | 49.262 | 15.977 | 53.201 | 1.00 | 45.24 |
| 439 | N | VAL | 56 | 49.437 | 13.942 | 54.160 | 1.00 | 82.87 |
| 440 | CA | VAL | 56 | 49.730 | 13.244 | 52.923 | 1.00 | 82.87 |
| 441 | CB | VAL | 56 | 50.831 | 12.192 | 53.155 | 1.00 | 72.67 |
| 442 | CG1 | VAL | 56 | 51.176 | 11.489 | 51.856 | 1.00 | 72.67 |
| 443 | CG2 | VAL | 56 | 52.057 | 12.862 | 53.731 | 1.00 | 72.67 |
| 444 | C | VAL | 56 | 48.467 | 12.569 | 52.398 | 1.00 | 82.87 |
| 445 | O | VAL | 56 | 47.689 | 12.010 | 53.171 | 1.00 | 82.87 |
| 446 | N | ASN | 57 | 48.268 | 12.636 | 51.085 | 1.00 | 59.63 |
| 447 | CA | ASN | 57 | 47.099 | 12.059 | 50.438 | 1.00 | 59.63 |
| 448 | CB | ASN | 57 | 47.302 | 10.562 | 50.308 | 1.00 | 98.65 |
| 449 | CG | ASN | 57 | 48.632 | 10.237 | 49.655 | 1.00 | 98.65 |
| 450 | OD1 | ASN | 57 | 48.980 | 10.808 | 48.609 | 1.00 | 98.65 |
| 451 | ND2 | ASN | 57 | 49.391 | 9.331 | 50.267 | 1.00 | 98.65 |
| 452 | C | ASN | 57 | 45.864 | 12.412 | 51.249 | 1.00 | 59.63 |
| 453 | O | ASN | 57 | 45.322 | 11.593 | 51.998 | 1.00 | 59.63 |
| 454 | N | ALA | 58 | 45.443 | 13.665 | 51.090 | 1.00 | 71.80 |
| 455 | CA | ALA | 58 | 44.300 | 14.216 | 51.809 | 1.00 | 71.80 |
| 456 | CB | ALA | 58 | 43.991 | 15.617 | 51.310 | 1.00 | 87.99 |
| 457 | C | ALA | 58 | 43.073 | 13.358 | 51.690 | 1.00 | 71.80 |
| 458 | O | ALA | 58 | 42.783 | 12.818 | 50.625 | 1.00 | 71.80 |
| 459 | N | LYS | 59 | 42.363 | 13.240 | 52.805 | 1.00 | 72.92 |
| 460 | CA | LYS | 59 | 41.128 | 12.467 | 52.885 | 1.00 | 72.92 |
| 461 | CB | LYS | 59 | 41.293 | 11.281 | 53.830 | 1.00 | 124.59 |
| 462 | CG | LYS | 59 | 42.422 | 10.361 | 53.457 | 1.00 | 124.59 |
| 463 | CD | LYS | 59 | 42.480 | 9.187 | 54.387 | 1.00 | 124.59 |
| 464 | CE | LYS | 59 | 43.356 | 8.128 | 53.783 | 1.00 | 124.59 |
| 465 | NZ | LYS | 59 | 43.282 | 6.869 | 54.561 | 1.00 | 124.59 |
| 466 | C | LYS | 59 | 40.082 | 13.397 | 53.457 | 1.00 | 72.92 |
| 467 | O | LYS | 59 | 40.356 | 14.132 | 54.401 | 1.00 | 72.92 |
| 468 | N | PHE | 60 | 38.887 | 13.381 | 52.897 | 1.00 | 56.87 |
| 469 | CA | PHE | 60 | 37.848 | 14.255 | 53.416 | 1.00 | 56.87 |
| 470 | CB | PHE | 60 | 36.488 | 13.707 | 53.028 | 1.00 | 109.01 |
| 471 | CG | PHE | 60 | 36.292 | 13.613 | 51.562 | 1.00 | 109.01 |
| 472 | CD1 | PHE | 60 | 35.499 | 12.629 | 51.015 | 1.00 | 109.01 |
| 473 | CD2 | PHE | 60 | 36.957 | 14.485 | 50.714 | 1.00 | 109.01 |
| 474 | CE1 | PHE | 60 | 35.336 | 12.536 | 49.634 | 1.00 | 109.01 |
| 475 | CE2 | PHE | 60 | 36.803 | 14.404 | 49.337 | 1.00 | 109.01 |
| 476 | CZ | PHE | 60 | 36.006 | 13.414 | 48.793 | 1.00 | 109.01 |
| 477 | C | PHE | 60 | 37.930 | 14.414 | 54.928 | 1.00 | 56.87 |
| 478 | O | PHE | 60 | 37.628 | 15.478 | 55.475 | 1.00 | 56.87 |
| 479 | N | GLU | 61 | 38.352 | 13.350 | 55.598 | 1.00 | 64.50 |
| 480 | CA | GLU | 61 | 38.460 | 13.336 | 57.054 | 1.00 | 64.50 |
| 481 | CB | GLU | 61 | 38.892 | 11.953 | 57.542 | 1.00 | 154.96 |
| 482 | CG | GLU | 61 | 37.855 | 10.870 | 57.334 | 1.00 | 154.96 |
| 483 | CD | GLU | 61 | 37.517 | 10.655 | 55.871 | 1.00 | 154.96 |
| 484 | OE1 | GLU | 61 | 38.443 | 10.355 | 55.087 | 1.00 | 154.96 |
| 485 | OE2 | GLU | 61 | 36.328 | 10.784 | 55.505 | 1.00 | 154.96 |
| 486 | C | GLU | 61 | 39.436 | 14.356 | 57.576 | 1.00 | 64.50 |
| 487 | O | GLU | 61 | 39.351 | 14.765 | 58.731 | 1.00 | 64.50 |
| 488 | N | ASP | 62 | 40.371 | 14.745 | 56.713 | 1.00 | 71.49 |
| 489 | CA | ASP | 62 | 41.404 | 15.723 | 57.035 | 1.00 | 71.49 |
| 490 | CB | ASP | 62 | 42.574 | 15.543 | 56.079 | 1.00 | 78.68 |
| 491 | CG | ASP | 62 | 43.412 | 14.316 | 56.422 | 1.00 | 78.68 |
| 492 | OD1 | ASP | 62 | 43.641 | 14.093 | 57.639 | 1.00 | 78.68 |
| 493 | OD2 | ASP | 62 | 43.851 | 13.594 | 55.485 | 1.00 | 78.68 |
| 494 | C | ASP | 62 | 40.883 | 17.160 | 57.000 | 1.00 | 71.49 |
| 495 | O | ASP | 62 | 41.574 | 18.102 | 57.400 | 1.00 | 71.49 |
| 496 | N | SER | 63 | 39.654 | 17.326 | 56.523 | 1.00 | 52.56 |
| 497 | CA | SER | 63 | 39.056 | 18.637 | 56.490 | 1.00 | 52.56 |
| 498 | CB | SER | 63 | 37.722 | 18.586 | 55.773 | 1.00 | 54.10 |
| 499 | OG | SER | 63 | 37.914 | 18.110 | 54.456 | 1.00 | 54.10 |
| 500 | C | SER | 63 | 38.850 | 19.020 | 57.936 | 1.00 | 52.56 |
| 501 | O | SER | 63 | 39.035 | 18.202 | 58.826 | 1.00 | 52.56 |
| 502 | N | GLY | 64 | 38.479 | 20.268 | 58.171 | 1.00 | 71.62 |
| 503 | CA | GLY | 64 | 38.243 | 20.690 | 59.528 | 1.00 | 71.62 |
| 504 | C | GLY | 64 | 39.138 | 21.809 | 59.962 | 1.00 | 71.62 |
| 505 | O | GLY | 64 | 39.954 | 22.299 | 59.196 | 1.00 | 71.62 |
| 506 | N | GLU | 65 | 38.957 | 22.205 | 61.211 | 1.00 | 87.67 |
| 507 | CA | GLU | 65 | 39.712 | 23.270 | 61.833 | 1.00 | 87.67 |
| 508 | CB | GLU | 65 | 38.887 | 23.868 | 62.967 | 1.00 | 86.11 |
| 509 | CG | GLU | 65 | 39.602 | 24.889 | 63.834 | 1.00 | 86.11 |
| 510 | CD | GLU | 65 | 38.934 | 25.056 | 65.194 | 1.00 | 86.11 |
| 511 | OE1 | GLU | 65 | 39.124 | 24.172 | 66.068 | 1.00 | 86.11 |
| 512 | OE2 | GLU | 65 | 38.210 | 26.059 | 65.386 | 1.00 | 86.11 |
| 513 | C | GLU | 65 | 40.988 | 22.684 | 62.395 | 1.00 | 87.67 |
| 514 | O | GLU | 65 | 41.072 | 21.485 | 62.640 | 1.00 | 87.67 |
| 515 | N | TYR | 66 | 41.980 | 23.539 | 62.598 | 1.00 | 99.15 |
| 516 | CA | TYR | 66 | 43.258 | 23.129 | 63.151 | 1.00 | 99.15 |
| 517 | CB | TYR | 66 | 44.246 | 22.786 | 62.044 | 1.00 | 44.24 |
| 518 | CG | TYR | 66 | 44.017 | 21.547 | 61.224 | 1.00 | 44.24 |
| 519 | CD1 | TYR | 66 | 43.042 | 21.512 | 60.225 | 1.00 | 44.24 |
| 520 | CE1 | TYR | 66 | 42.884 | 20.381 | 59.401 | 1.00 | 44.24 |
| 521 | CD2 | TYR | 66 | 44.838 | 20.429 | 61.399 | 1.00 | 44.24 |
| 522 | CE2 | TYR | 66 | 44.700 | 19.299 | 60.600 | 1.00 | 44.24 |
| 523 | CZ | TYR | 66 | 43.719 | 19.270 | 59.596 | 1.00 | 44.24 |
| 524 | OH | TYR | 66 | 43.560 | 18.121 | 58.822 | 1.00 | 44.24 |
| 525 | C | TYR | 66 | 43.848 | 24.303 | 63.935 | 1.00 | 99.15 |
| 526 | O | TYR | 66 | 43.714 | 25.462 | 63.526 | 1.00 | 99.15 |
| 527 | N | LYS | 67 | 44.502 | 23.996 | 65.052 | 1.00 | 76.23 |
| 528 | CA | LYS | 67 | 45.158 | 25.008 | 65.883 | 1.00 | 76.23 |
| 529 | CB | LYS | 67 | 44.357 | 25.311 | 67.152 | 1.00 | 84.41 |
| 530 | CG | LYS | 67 | 42.934 | 25.780 | 66.944 | 1.00 | 84.41 |
| 531 | CD | LYS | 67 | 42.212 | 25.802 | 68.294 | 1.00 | 84.41 |
| 532 | CE | LYS | 67 | 40.725 | 26.104 | 68.164 | 1.00 | 84.41 |
| 533 | NZ | LYS | 67 | 40.042 | 25.844 | 69.456 | 1.00 | 84.41 |
| 534 | C | LYS | 67 | 46.477 | 24.393 | 66.303 | 1.00 | 76.23 |
| 535 | O | LYS | 67 | 46.621 | 23.170 | 66.300 | 1.00 | 76.23 |
| 536 | N | CYS | 68 | 47.449 | 25.235 | 66.625 | 1.00 | 101.62 |
| 537 | CA | CYS | 68 | 48.729 | 24.757 | 67.110 | 1.00 | 101.62 |
| 538 | C | CYS | 68 | 49.018 | 25.644 | 68.296 | 1.00 | 101.62 |
| 539 | O | CYS | 68 | 48.586 | 26.797 | 68.335 | 1.00 | 101.62 |
| 540 | CB | CYS | 68 | 49.836 | 24.883 | 66.058 | 1.00 | 149.58 |
| 541 | SG | CYS | 68 | 50.287 | 26.560 | 65.501 | 1.00 | 149.58 |
| 542 | N | GLN | 69 | 49.716 | 25.099 | 69.281 | 1.00 | 93.84 |
| 543 | CA | GLN | 69 | 50.045 | 25.874 | 70.458 | 1.00 | 93.84 |
| 544 | CB | GLN | 69 | 48.935 | 25.756 | 71.497 | 1.00 | 108.47 |
| 545 | CG | GLN | 69 | 49.325 | 26.336 | 72.838 | 1.00 | 108.47 |
| 546 | CD | GLN | 69 | 48.228 | 26.198 | 73.866 | 1.00 | 108.47 |
| 547 | OE1 | GLN | 69 | 47.421 | 25.266 | 73.801 | 1.00 | 108.47 |
| 548 | NE2 | GLN | 69 | 48.197 | 27.116 | 74.835 | 1.00 | 108.47 |
| 549 | C | GLN | 69 | 51.362 | 25.450 | 71.087 | 1.00 | 93.84 |
| 550 | O | GLN | 69 | 51.813 | 24.310 | 70.919 | 1.00 | 93.84 |
| 551 | N | HIS | 70 | 51.986 | 26.399 | 71.778 | 1.00 | 149.64 |
| 552 | CA | HIS | 70 | 53.221 | 26.162 | 72.504 | 1.00 | 149.64 |
| 553 | CB | HIS | 70 | 54.272 | 27.216 | 72.161 | 1.00 | 188.52 |
| 554 | CG | HIS | 70 | 54.952 | 26.965 | 70.857 | 1.00 | 188.52 |
| 555 | CD2 | HIS | 70 | 54.804 | 25.981 | 69.944 | 1.00 | 188.52 |
| 556 | ND1 | HIS | 70 | 55.951 | 27.788 | 70.358 | 1.00 | 188.52 |
| 557 | CE1 | HIS | 70 | 56.375 | 27.316 | 69.210 | 1.00 | 188.52 |
| 558 | NE2 | HIS | 70 | 55.693 | 26.212 | 68.928 | 1.00 | 188.52 |
| 559 | C | HIS | 70 | 52.788 | 26.284 | 73.952 | 1.00 | 149.64 |
| 560 | O | HIS | 70 | 52.566 | 27.385 | 74.454 | 1.00 | 149.64 |
| 561 | N | GLN | 71 | 52.642 | 25.132 | 74.599 | 1.00 | 126.80 |
| 562 | CA | GLN | 71 | 52.191 | 25.045 | 75.981 | 1.00 | 126.80 |
| 563 | CB | GLN | 71 | 52.974 | 23.964 | 76.726 | 1.00 | 175.94 |
| 564 | CG | GLN | 71 | 52.367 | 23.589 | 78.072 | 1.00 | 175.94 |
| 565 | CD | GLN | 71 | 53.119 | 22.461 | 78.752 | 1.00 | 175.94 |
| 566 | OE1 | GLN | 71 | 54.130 | 21.984 | 78.241 | 1.00 | 175.94 |
| 567 | NE2 | GLN | 71 | 52.631 | 22.031 | 79.909 | 1.00 | 175.94 |
| 568 | C | GLN | 71 | 52.267 | 26.359 | 76.745 | 1.00 | 126.80 |
| 569 | O | GLN | 71 | 53.343 | 26.934 | 76.927 | 1.00 | 126.80 |
| 570 | N | GLN | 72 | 51.096 | 26.825 | 77.169 | 1.00 | 138.09 |
| 571 | CA | GLN | 72 | 50.936 | 28.060 | 77.933 | 1.00 | 138.09 |
| 572 | CB | GLN | 72 | 51.762 | 28.017 | 79.214 | 1.00 | 197.82 |
| 573 | CG | GLN | 72 | 51.264 | 27.031 | 80.242 | 1.00 | 197.82 |
| 574 | CD | GLN | 72 | 49.767 | 27.138 | 80.538 | 1.00 | 197.82 |
| 575 | OE1 | GLN | 72 | 49.158 | 28.202 | 80.397 | 1.00 | 197.82 |
| 576 | NE2 | GLN | 72 | 49.175 | 26.028 | 80.973 | 1.00 | 197.82 |
| 577 | C | GLN | 72 | 51.252 | 29.347 | 77.196 | 1.00 | 138.09 |
| 578 | O | GLN | 72 | 51.503 | 30.377 | 77.821 | 1.00 | 138.09 |
| 579 | N | VAL | 73 | 51.243 | 29.291 | 75.871 | 1.00 | 127.03 |
| 580 | CA | VAL | 73 | 51.505 | 30.477 | 75.070 | 1.00 | 127.03 |
| 581 | CB | VAL | 73 | 52.817 | 30.340 | 74.250 | 1.00 | 90.79 |
| 582 | CG1 | VAL | 73 | 53.197 | 31.683 | 73.626 | 1.00 | 90.79 |
| 583 | CG2 | VAL | 73 | 53.943 | 29.826 | 75.148 | 1.00 | 90.79 |
| 584 | C | VAL | 73 | 50.314 | 30.625 | 74.134 | 1.00 | 127.03 |
| 585 | O | VAL | 73 | 50.467 | 30.866 | 72.936 | 1.00 | 127.03 |
| 586 | N | ASN | 74 | 49.119 | 30.457 | 74.693 | 1.00 | 129.72 |
| 587 | CA | ASN | 74 | 47.890 | 30.577 | 73.920 | 1.00 | 129.72 |
| 588 | CB | ASN | 74 | 47.721 | 31.993 | 73.390 | 1.00 | 137.31 |
| 589 | CG | ASN | 74 | 47.576 | 33.011 | 74.476 | 1.00 | 137.31 |
| 590 | OD1 | ASN | 74 | 48.512 | 33.785 | 74.713 | 1.00 | 137.31 |
| 591 | ND2 | ASN | 74 | 46.400 | 33.015 | 75.123 | 1.00 | 137.31 |
| 592 | C | ASN | 74 | 47.820 | 29.655 | 72.709 | 1.00 | 129.72 |
| 593 | O | ASN | 74 | 48.836 | 29.209 | 72.176 | 1.00 | 129.72 |
| 594 | N | GLU | 75 | 46.600 | 29.404 | 72.254 | 1.00 | 128.29 |
| 595 | CA | GLU | 75 | 46.377 | 28.580 | 71.080 | 1.00 | 128.29 |
| 596 | CB | GLU | 75 | 44.998 | 27.934 | 71.175 | 1.00 | 132.07 |
| 597 | CG | GLU | 75 | 44.672 | 27.459 | 72.584 | 1.00 | 132.07 |
| 598 | CD | GLU | 75 | 43.266 | 26.922 | 72.700 | 1.00 | 132.07 |
| 599 | OE1 | GLU | 75 | 42.439 | 27.252 | 71.821 | 1.00 | 132.07 |
| 600 | OE2 | GLU | 75 | 42.989 | 26.185 | 73.673 | 1.00 | 132.07 |
| 601 | C | GLU | 75 | 46.446 | 29.547 | 69.897 | 1.00 | 128.29 |
| 602 | O | GLU | 75 | 46.432 | 30.757 | 70.089 | 1.00 | 128.29 |
| 603 | N | SER | 76 | 46.545 | 29.033 | 68.681 | 1.00 | 77.19 |
| 604 | CA | SER | 76 | 46.595 | 29.901 | 67.506 | 1.00 | 77.19 |
| 605 | CB | SER | 76 | 47.321 | 29.207 | 66.363 | 1.00 | 54.46 |
| 606 | OG | SER | 76 | 46.418 | 28.351 | 65.649 | 1.00 | 54.46 |
| 607 | C | SER | 76 | 45.175 | 30.169 | 67.033 | 1.00 | 77.19 |
| 608 | O | SER | 76 | 44.208 | 29.659 | 67.603 | 1.00 | 77.19 |
| 609 | N | GLU | 77 | 45.038 | 30.966 | 65.983 | 1.00 | 74.40 |
| 610 | CA | GLU | 77 | 43.711 | 31.198 | 65.444 | 1.00 | 74.40 |
| 611 | CB | GLU | 77 | 43.652 | 32.492 | 64.632 | 1.00 | 153.94 |
| 612 | CG | GLU | 77 | 43.693 | 33.746 | 65.491 | 1.00 | 153.94 |
| 613 | CD | GLU | 77 | 42.624 | 33.749 | 66.578 | 1.00 | 153.94 |
| 614 | OE1 | GLU | 77 | 41.418 | 33.712 | 66.237 | 1.00 | 153.94 |
| 615 | OE2 | GLU | 77 | 42.992 | 33.787 | 67.775 | 1.00 | 153.94 |
| 616 | C | GLU | 77 | 43.460 | 29.998 | 64.558 | 1.00 | 74.40 |
| 617 | O | GLU | 77 | 44.375 | 29.495 | 63.913 | 1.00 | 74.40 |
| 618 | N | PRO | 78 | 42.215 | 29.512 | 64.527 | 1.00 | 88.95 |
| 619 | CD | PRO | 78 | 41.068 | 29.938 | 65.344 | 1.00 | 122.79 |
| 620 | CA | PRO | 78 | 41.857 | 28.352 | 63.712 | 1.00 | 88.95 |
| 621 | CB | PRO | 78 | 40.385 | 28.138 | 64.046 | 1.00 | 122.79 |
| 622 | CG | PRO | 78 | 40.268 | 28.670 | 65.428 | 1.00 | 122.79 |
| 623 | C | PRO | 78 | 42.061 | 28.542 | 62.221 | 1.00 | 88.95 |
| 624 | O | PRO | 78 | 41.901 | 29.640 | 61.688 | 1.00 | 88.95 |
| 625 | N | VAL | 79 | 42.415 | 27.447 | 61.560 | 1.00 | 101.27 |
| 626 | CA | VAL | 79 | 42.604 | 27.427 | 60.119 | 1.00 | 101.27 |
| 627 | CB | VAL | 79 | 44.042 | 27.114 | 59.735 | 1.00 | 79.48 |
| 628 | CG1 | VAL | 79 | 44.177 | 27.097 | 58.226 | 1.00 | 79.48 |
| 629 | CG2 | VAL | 79 | 44.962 | 28.130 | 60.341 | 1.00 | 79.48 |
| 630 | C | VAL | 79 | 41.727 | 26.304 | 59.592 | 1.00 | 101.27 |
| 631 | O | VAL | 79 | 41.846 | 25.155 | 60.025 | 1.00 | 101.27 |
| 632 | N | TYR | 80 | 40.851 | 26.629 | 58.655 | 1.00 | 55.37 |
| 633 | CA | TYR | 80 | 39.973 | 25.618 | 58.115 | 1.00 | 55.37 |
| 634 | CB | TYR | 80 | 38.551 | 26.145 | 58.016 | 1.00 | 122.28 |
| 635 | CG | TYR | 80 | 38.004 | 26.497 | 59.364 | 1.00 | 122.28 |
| 636 | CD1 | TYR | 80 | 38.278 | 27.733 | 59.942 | 1.00 | 122.28 |
| 637 | CE1 | TYR | 80 | 37.817 | 28.049 | 61.208 | 1.00 | 122.28 |
| 638 | CD2 | TYR | 80 | 37.250 | 25.577 | 60.088 | 1.00 | 122.28 |
| 639 | CE2 | TYR | 80 | 36.782 | 25.877 | 61.357 | 1.00 | 122.28 |
| 640 | CZ | TYR | 80 | 37.069 | 27.117 | 61.915 | 1.00 | 122.28 |
| 641 | OH | TYR | 80 | 36.615 | 27.424 | 63.180 | 1.00 | 122.28 |
| 642 | C | TYR | 80 | 40.390 | 25.066 | 56.779 | 1.00 | 55.37 |
| 643 | O | TYR | 80 | 40.474 | 25.780 | 55.795 | 1.00 | 55.37 |
| 644 | N | LEU | 81 | 40.627 | 23.768 | 56.765 | 1.00 | 67.93 |
| 645 | CA | LEU | 81 | 41.026 | 23.065 | 55.578 | 1.00 | 67.93 |
| 646 | CB | LEU | 81 | 42.016 | 22.014 | 55.982 | 1.00 | 70.09 |
| 647 | CG | LEU | 81 | 42.558 | 21.223 | 54.819 | 1.00 | 70.09 |
| 648 | CD1 | LEU | 81 | 43.387 | 22.150 | 53.967 | 1.00 | 70.09 |
| 649 | CD2 | LEU | 81 | 43.411 | 20.066 | 55.338 | 1.00 | 70.09 |
| 650 | C | LEU | 81 | 39.781 | 22.405 | 55.006 | 1.00 | 67.93 |
| 651 | O | LEU | 81 | 38.856 | 22.098 | 55.763 | 1.00 | 67.93 |
| 652 | N | GLU | 82 | 39.732 | 22.188 | 53.691 | 1.00 | 70.25 |
| 653 | CA | GLU | 82 | 38.569 | 21.530 | 53.079 | 1.00 | 70.25 |
| 654 | CB | GLU | 82 | 37.486 | 22.562 | 52.737 | 1.00 | 131.12 |
| 655 | CG | GLU | 82 | 36.199 | 21.935 | 52.234 | 1.00 | 131.12 |
| 656 | CD | GLU | 82 | 34.994 | 22.847 | 52.359 | 1.00 | 131.12 |
| 657 | OE1 | GLU | 82 | 35.091 | 24.023 | 51.958 | 1.00 | 131.12 |
| 658 | OE2 | GLU | 82 | 33.940 | 22.384 | 52.847 | 1.00 | 131.12 |
| 659 | C | GLU | 82 | 38.957 | 20.709 | 51.843 | 1.00 | 70.25 |
| 660 | O | GLU | 82 | 39.425 | 21.261 | 50.850 | 1.00 | 70.25 |
| 661 | N | VAL | 83 | 38.769 | 19.386 | 51.918 | 1.00 | 52.36 |
| 662 | CA | VAL | 83 | 39.106 | 18.469 | 50.815 | 1.00 | 52.36 |
| 663 | CB | VAL | 83 | 39.452 | 17.094 | 51.350 | 1.00 | 43.44 |
| 664 | CG1 | VAL | 83 | 40.082 | 16.245 | 50.244 | 1.00 | 43.44 |
| 665 | CG2 | VAL | 83 | 40.359 | 17.241 | 52.544 | 1.00 | 43.44 |
| 666 | C | VAL | 83 | 37.995 | 18.302 | 49.768 | 1.00 | 52.36 |
| 667 | O | VAL | 83 | 36.852 | 18.022 | 50.105 | 1.00 | 52.36 |
| 668 | N | PHE | 84 | 38.342 | 18.479 | 48.498 | 1.00 | 63.83 |
| 669 | CA | PHE | 84 | 37.368 | 18.369 | 47.426 | 1.00 | 63.83 |
| 670 | CB | PHE | 84 | 37.359 | 19.633 | 46.562 | 1.00 | 62.38 |
| 671 | CG | PHE | 84 | 36.918 | 20.841 | 47.294 | 1.00 | 62.38 |
| 672 | CD1 | PHE | 84 | 37.692 | 21.345 | 48.329 | 1.00 | 62.38 |
| 673 | CD2 | PHE | 84 | 35.680 | 21.429 | 47.020 | 1.00 | 62.38 |
| 674 | CE1 | PHE | 84 | 37.244 | 22.418 | 49.092 | 1.00 | 62.38 |
| 675 | CE2 | PHE | 84 | 35.223 | 22.506 | 47.780 | 1.00 | 62.38 |
| 676 | CZ | PHE | 84 | 36.007 | 22.998 | 48.823 | 1.00 | 62.38 |
| 677 | C | PHE | 84 | 37.606 | 17.175 | 46.527 | 1.00 | 63.83 |
| 678 | O | PHE | 84 | 38.619 | 16.477 | 46.630 | 1.00 | 63.83 |
| 679 | N | SER | 85 | 36.635 | 16.952 | 45.650 | 1.00 | 70.22 |
| 680 | CA | SER | 85 | 36.663 | 15.891 | 44.671 | 1.00 | 70.22 |
| 681 | CB | SER | 85 | 36.006 | 14.615 | 45.197 | 1.00 | 107.42 |
| 682 | OG | SER | 85 | 36.099 | 13.567 | 44.235 | 1.00 | 107.42 |
| 683 | C | SER | 85 | 35.833 | 16.465 | 43.552 | 1.00 | 70.22 |
| 684 | O | SER | 85 | 34.608 | 16.501 | 43.625 | 1.00 | 70.22 |
| 685 | N | ASP | 86 | 36.513 | 16.953 | 42.528 | 1.00 | 30.45 |
| 686 | CA | ASP | 86 | 35.835 | 17.517 | 41.391 | 1.00 | 30.45 |
| 687 | CB | ASP | 86 | 35.151 | 18.796 | 41.799 | 1.00 | 66.75 |
| 688 | CG | ASP | 86 | 34.005 | 19.121 | 40.908 | 1.00 | 66.75 |
| 689 | OD1 | ASP | 86 | 34.186 | 19.182 | 39.664 | 1.00 | 66.75 |
| 690 | OD2 | ASP | 86 | 32.909 | 19.311 | 41.455 | 1.00 | 66.75 |
| 691 | C | ASP | 86 | 36.876 | 17.768 | 40.303 | 1.00 | 30.45 |
| 692 | O | ASP | 86 | 38.074 | 17.603 | 40.551 | 1.00 | 30.45 |
| 693 | N | TRP | 87 | 36.427 | 18.122 | 39.101 | 1.00 | 54.88 |
| 694 | CA | TRP | 87 | 37.329 | 18.392 | 37.986 | 1.00 | 54.88 |
| 695 | CB | TRP | 87 | 36.541 | 18.398 | 36.686 | 1.00 | 120.63 |
| 696 | CG | TRP | 87 | 36.228 | 17.051 | 36.187 | 1.00 | 120.63 |
| 697 | CD2 | TRP | 87 | 35.123 | 16.242 | 36.559 | 1.00 | 120.63 |
| 698 | CE2 | TRP | 87 | 35.205 | 15.040 | 35.822 | 1.00 | 120.63 |
| 699 | CE3 | TRP | 87 | 34.043 | 16.411 | 37.446 | 1.00 | 120.63 |
| 700 | CD1 | TRP | 87 | 36.936 | 16.335 | 35.269 | 1.00 | 120.63 |
| 701 | NE1 | TRP | 87 | 36.336 | 15.125 | 35.036 | 1.00 | 120.63 |
| 702 | CZ2 | TRP | 87 | 34.277 | 14.011 | 35.939 | 1.00 | 120.63 |
| 703 | CZ3 | TRP | 87 | 33.109 | 15.387 | 37.568 | 1.00 | 120.63 |
| 704 | CH2 | TRP | 87 | 33.231 | 14.202 | 36.813 | 1.00 | 120.63 |
| 705 | C | TRP | 87 | 38.051 | 19.726 | 38.150 | 1.00 | 54.88 |
| 706 | O | TRP | 87 | 39.248 | 19.818 | 37.884 | 1.00 | 54.88 |
| 707 | N | LEU | 88 | 37.307 | 20.751 | 38.578 | 1.00 | 42.94 |
| 708 | CA | LEU | 88 | 37.831 | 22.113 | 38.806 | 1.00 | 42.94 |
| 709 | CB | LEU | 88 | 37.131 | 23.157 | 37.946 | 1.00 | 57.40 |
| 710 | CG | LEU | 88 | 37.526 | 23.193 | 36.485 | 1.00 | 57.40 |
| 711 | CD1 | LEU | 88 | 36.761 | 24.330 | 35.822 | 1.00 | 57.40 |
| 712 | CD2 | LEU | 88 | 39.030 | 23.370 | 36.354 | 1.00 | 57.40 |
| 713 | C | LEU | 88 | 37.671 | 22.549 | 40.235 | 1.00 | 42.94 |
| 714 | O | LEU | 88 | 36.666 | 22.301 | 40.870 | 1.00 | 42.94 |
| 715 | N | LEU | 89 | 38.681 | 23.225 | 40.733 | 1.00 | 58.27 |
| 716 | CA | LEU | 89 | 38.667 | 23.700 | 42.085 | 1.00 | 58.27 |
| 717 | CB | LEU | 89 | 39.556 | 22.835 | 42.951 | 1.00 | 5.00 |
| 718 | CG | LEU | 89 | 39.739 | 23.331 | 44.403 | 1.00 | 5.00 |
| 719 | CD1 | LEU | 89 | 38.371 | 23.632 | 45.112 | 1.00 | 5.00 |
| 720 | CD2 | LEU | 89 | 40.628 | 22.281 | 45.195 | 1.00 | 5.00 |
| 721 | C | LEU | 89 | 39.237 | 25.082 | 42.056 | 1.00 | 58.27 |
| 722 | O | LEU | 89 | 40.346 | 25.279 | 41.555 | 1.00 | 58.27 |
| 723 | N | LEU | 90 | 38.486 | 26.043 | 42.582 | 1.00 | 24.40 |
| 724 | CA | LEU | 90 | 38.995 | 27.407 | 42.636 | 1.00 | 24.40 |
| 725 | CB | LEU | 90 | 37.861 | 28.425 | 42.585 | 1.00 | 63.96 |
| 726 | CG | LEU | 90 | 38.331 | 29.867 | 42.711 | 1.00 | 63.96 |
| 727 | CD1 | LEU | 90 | 39.284 | 30.173 | 41.570 | 1.00 | 63.96 |
| 728 | CD2 | LEU | 90 | 37.134 | 30.803 | 42.692 | 1.00 | 63.96 |
| 729 | C | LEU | 90 | 39.702 | 27.535 | 43.969 | 1.00 | 24.40 |
| 730 | O | LEU | 90 | 39.057 | 27.496 | 45.000 | 1.00 | 24.40 |
| 731 | N | GLN | 91 | 41.017 | 27.690 | 43.970 | 1.00 | 52.44 |
| 732 | CA | GLN | 91 | 41.721 | 27.825 | 45.234 | 1.00 | 52.44 |
| 733 | CB | GLN | 91 | 43.018 | 27.058 | 45.151 | 1.00 | 32.27 |
| 734 | CG | GLN | 91 | 42.820 | 25.718 | 44.534 | 1.00 | 32.27 |
| 735 | CD | GLN | 91 | 44.020 | 24.841 | 44.727 | 1.00 | 32.27 |
| 736 | OE1 | GLN | 91 | 45.010 | 24.901 | 43.957 | 1.00 | 32.27 |
| 737 | NE2 | GLN | 91 | 43.962 | 24.016 | 45.788 | 1.00 | 32.27 |
| 738 | C | GLN | 91 | 41.981 | 29.299 | 45.554 | 1.00 | 52.44 |
| 739 | O | GLN | 91 | 42.060 | 30.142 | 44.657 | 1.00 | 52.44 |
| 740 | N | ALA | 92 | 42.075 | 29.633 | 46.831 | 1.00 | 55.47 |
| 741 | CA | ALA | 92 | 42.345 | 31.018 | 47.175 | 1.00 | 55.47 |
| 742 | CB | ALA | 92 | 41.076 | 31.735 | 47.638 | 1.00 | 37.36 |
| 743 | C | ALA | 92 | 43.381 | 31.100 | 48.246 | 1.00 | 55.47 |
| 744 | O | ALA | 92 | 43.384 | 30.316 | 49.183 | 1.00 | 55.47 |
| 745 | N | SER | 93 | 44.263 | 32.064 | 48.077 | 1.00 | 51.36 |
| 746 | CA | SER | 93 | 45.329 | 32.345 | 49.008 | 1.00 | 51.36 |
| 747 | CB | SER | 93 | 45.997 | 33.647 | 48.583 | 1.00 | 54.66 |
| 748 | OG | SER | 93 | 45.009 | 34.650 | 48.346 | 1.00 | 54.66 |
| 749 | C | SER | 93 | 44.690 | 32.509 | 50.378 | 1.00 | 51.36 |
| 750 | O | SER | 93 | 45.238 | 32.104 | 51.395 | 1.00 | 51.36 |
| 751 | N | ALA | 94 | 43.521 | 33.124 | 50.397 | 1.00 | 46.22 |
| 752 | CA | ALA | 94 | 42.822 | 33.313 | 51.645 | 1.00 | 46.22 |
| 753 | CB | ALA | 94 | 43.522 | 34.352 | 52.496 | 1.00 | 74.92 |
| 754 | C | ALA | 94 | 41.393 | 33.723 | 51.340 | 1.00 | 46.22 |
| 755 | O | ALA | 94 | 41.111 | 34.357 | 50.317 | 1.00 | 46.22 |
| 756 | N | GLU | 95 | 40.489 | 33.326 | 52.231 | 1.00 | 89.58 |
| 757 | CA | GLU | 95 | 39.074 | 33.605 | 52.065 | 1.00 | 89.58 |
| 758 | CB | GLU | 95 | 38.283 | 32.448 | 52.662 | 1.00 | 145.77 |
| 759 | CG | GLU | 95 | 38.549 | 31.139 | 51.938 | 1.00 | 145.77 |
| 760 | CD | GLU | 95 | 37.950 | 29.953 | 52.650 | 1.00 | 145.77 |
| 761 | OE1 | GLU | 95 | 38.060 | 28.826 | 52.118 | 1.00 | 145.77 |
| 762 | OE2 | GLU | 95 | 37.373 | 30.145 | 53.742 | 1.00 | 145.77 |
| 763 | C | GLU | 95 | 38.662 | 34.938 | 52.678 | 1.00 | 89.58 |
| 764 | O | GLU | 95 | 37.661 | 35.546 | 52.289 | 1.00 | 89.58 |
| 765 | N | VAL | 96 | 39.453 | 35.379 | 53.644 | 1.00 | 75.49 |
| 766 | CA | VAL | 96 | 39.233 | 36.643 | 54.322 | 1.00 | 75.49 |
| 767 | CB | VAL | 96 | 38.995 | 36.436 | 55.819 | 1.00 | 87.57 |
| 768 | CG1 | VAL | 96 | 38.710 | 37.771 | 56.480 | 1.00 | 87.57 |
| 769 | CG2 | VAL | 96 | 37.847 | 35.467 | 56.032 | 1.00 | 87.57 |
| 770 | C | VAL | 96 | 40.537 | 37.402 | 54.128 | 1.00 | 75.49 |
| 771 | O | VAL | 96 | 41.578 | 37.016 | 54.665 | 1.00 | 75.49 |
| 772 | N | VAL | 97 | 40.482 | 38.471 | 53.345 | 1.00 | 96.50 |
| 773 | CA | VAL | 97 | 41.670 | 39.254 | 53.066 | 1.00 | 96.50 |
| 774 | CB | VAL | 97 | 41.929 | 39.335 | 51.553 | 1.00 | 66.57 |
| 775 | CG1 | VAL | 97 | 42.920 | 40.443 | 51.259 | 1.00 | 66.57 |
| 776 | CG2 | VAL | 97 | 42.472 | 38.004 | 51.049 | 1.00 | 66.57 |
| 777 | C | VAL | 97 | 41.598 | 40.665 | 53.613 | 1.00 | 96.50 |
| 778 | O | VAL | 97 | 40.583 | 41.352 | 53.473 | 1.00 | 96.50 |
| 779 | N | MET | 98 | 42.693 | 41.088 | 54.237 | 1.00 | 97.55 |
| 780 | CA | MET | 98 | 42.784 | 42.425 | 54.799 | 1.00 | 97.55 |
| 781 | CB | MET | 98 | 43.870 | 42.482 | 55.887 | 1.00 | 148.78 |
| 782 | CG | MET | 98 | 43.567 | 41.601 | 57.108 | 1.00 | 148.78 |
| 783 | SD | MET | 98 | 44.803 | 41.635 | 58.454 | 1.00 | 148.78 |
| 784 | CE | MET | 98 | 45.665 | 40.108 | 58.182 | 1.00 | 148.78 |
| 785 | C | MET | 98 | 43.098 | 43.411 | 53.682 | 1.00 | 97.55 |
| 786 | O | MET | 98 | 44.143 | 43.318 | 53.034 | 1.00 | 97.55 |
| 787 | N | GLU | 99 | 42.161 | 44.329 | 53.455 | 1.00 | 85.18 |
| 788 | CA | GLU | 99 | 42.265 | 45.380 | 52.450 | 1.00 | 85.18 |
| 789 | CB | GLU | 99 | 41.565 | 46.635 | 52.988 | 1.00 | 186.55 |
| 790 | CG | GLU | 99 | 41.620 | 47.868 | 52.109 | 1.00 | 186.55 |
| 791 | CD | GLU | 99 | 40.716 | 48.978 | 52.629 | 1.00 | 186.55 |
| 792 | OE1 | GLU | 99 | 40.827 | 49.337 | 53.823 | 1.00 | 186.55 |
| 793 | OE2 | GLU | 99 | 39.894 | 49.490 | 51.839 | 1.00 | 186.55 |
| 794 | C | GLU | 99 | 43.724 | 45.677 | 52.137 | 1.00 | 85.18 |
| 795 | O | GLU | 99 | 44.605 | 45.506 | 52.990 | 1.00 | 85.18 |
| 796 | N | GLY | 100 | 43.991 | 46.106 | 50.909 | 1.00 | 64.51 |
| 797 | CA | GLY | 100 | 45.362 | 46.432 | 50.549 | 1.00 | 64.51 |
| 798 | C | GLY | 100 | 46.371 | 45.293 | 50.459 | 1.00 | 64.51 |
| 799 | O | GLY | 100 | 47.508 | 45.528 | 50.054 | 1.00 | 64.51 |
| 800 | N | GLN | 101 | 45.987 | 44.075 | 50.839 | 1.00 | 91.02 |
| 801 | CA | GLN | 101 | 46.896 | 42.930 | 50.742 | 1.00 | 91.02 |
| 802 | CB | GLN | 101 | 46.631 | 41.933 | 51.875 | 1.00 | 93.41 |
| 803 | CG | GLN | 101 | 47.181 | 42.383 | 53.207 | 1.00 | 93.41 |
| 804 | CD | GLN | 101 | 48.671 | 42.663 | 53.133 | 1.00 | 93.41 |
| 805 | OE1 | GLN | 101 | 49.117 | 43.530 | 52.384 | 1.00 | 93.41 |
| 806 | NE2 | GLN | 101 | 49.448 | 41.926 | 53.906 | 1.00 | 93.41 |
| 807 | C | GLN | 101 | 46.716 | 42.260 | 49.373 | 1.00 | 91.02 |
| 808 | O | GLN | 101 | 45.909 | 42.712 | 48.567 | 1.00 | 91.02 |
| 809 | N | PRO | 102 | 47.484 | 41.195 | 49.078 | 1.00 | 67.73 |
| 810 | CD | PRO | 102 | 48.769 | 40.802 | 49.675 | 1.00 | 29.55 |
| 811 | CA | PRO | 102 | 47.324 | 40.547 | 47.778 | 1.00 | 67.73 |
| 812 | CB | PRO | 102 | 48.730 | 40.145 | 47.432 | 1.00 | 29.55 |
| 813 | CG | PRO | 102 | 49.248 | 39.718 | 48.721 | 1.00 | 29.55 |
| 814 | C | PRO | 102 | 46.398 | 39.353 | 47.784 | 1.00 | 67.73 |
| 815 | O | PRO | 102 | 46.385 | 38.564 | 48.719 | 1.00 | 67.73 |
| 816 | N | LEU | 103 | 45.636 | 39.227 | 46.707 | 1.00 | 40.45 |
| 817 | CA | LEU | 103 | 44.688 | 38.154 | 46.552 | 1.00 | 40.45 |
| 818 | CB | LEU | 103 | 43.308 | 38.743 | 46.277 | 1.00 | 52.62 |
| 819 | CG | LEU | 103 | 42.141 | 37.774 | 46.464 | 1.00 | 52.62 |
| 820 | CD1 | LEU | 103 | 42.345 | 36.942 | 47.721 | 1.00 | 52.62 |
| 821 | CD2 | LEU | 103 | 40.852 | 38.574 | 46.533 | 1.00 | 52.62 |
| 822 | C | LEU | 103 | 45.115 | 37.227 | 45.415 | 1.00 | 40.45 |
| 823 | O | LEU | 103 | 45.314 | 37.665 | 44.280 | 1.00 | 40.45 |
| 824 | N | PHE | 104 | 45.274 | 35.945 | 45.739 | 1.00 | 56.34 |
| 825 | CA | PHE | 104 | 45.680 | 34.942 | 44.762 | 1.00 | 56.34 |
| 826 | CB | PHE | 104 | 46.898 | 34.159 | 45.251 | 1.00 | 133.82 |
| 827 | CG | PHE | 104 | 48.053 | 35.016 | 45.592 | 1.00 | 133.82 |
| 828 | CD1 | PHE | 104 | 48.298 | 35.367 | 46.905 | 1.00 | 133.82 |
| 829 | CD2 | PHE | 104 | 48.870 | 35.528 | 44.594 | 1.00 | 133.82 |
| 830 | CE1 | PHE | 104 | 49.344 | 36.223 | 47.229 | 1.00 | 133.82 |
| 831 | CE2 | PHE | 104 | 49.924 | 36.389 | 44.906 | 1.00 | 133.82 |
| 832 | CZ | PHE | 104 | 50.161 | 36.738 | 46.224 | 1.00 | 133.82 |
| 833 | C | PHE | 104 | 44.570 | 33.958 | 44.477 | 1.00 | 56.34 |
| 834 | O | PHE | 104 | 43.923 | 33.468 | 45.388 | 1.00 | 56.34 |
| 835 | N | LEU | 105 | 44.363 | 33.668 | 43.202 | 1.00 | 51.84 |
| 836 | CA | LEU | 105 | 43.341 | 32.730 | 42.792 | 1.00 | 51.84 |
| 837 | CB | LEU | 105 | 42.199 | 33.440 | 42.086 | 1.00 | 27.36 |
| 838 | CG | LEU | 105 | 41.385 | 34.348 | 42.989 | 1.00 | 27.36 |
| 839 | CD1 | LEU | 105 | 40.132 | 34.710 | 42.214 | 1.00 | 27.36 |
| 840 | CD2 | LEU | 105 | 40.988 | 33.638 | 44.293 | 1.00 | 27.36 |
| 841 | C | LEU | 105 | 43.897 | 31.690 | 41.857 | 1.00 | 51.84 |
| 842 | O | LEU | 105 | 44.802 | 31.948 | 41.068 | 1.00 | 51.84 |
| 843 | N | ARG | 106 | 43.337 | 30.499 | 41.929 | 1.00 | 60.02 |
| 844 | CA | ARG | 106 | 43.820 | 29.474 | 41.063 | 1.00 | 60.02 |
| 845 | CB | ARG | 106 | 44.949 | 28.740 | 41.722 | 1.00 | 28.53 |
| 846 | CG | ARG | 106 | 45.476 | 27.618 | 40.886 | 1.00 | 28.53 |
| 847 | CD | ARG | 106 | 46.314 | 26.830 | 41.814 | 1.00 | 28.53 |
| 848 | NE | ARG | 106 | 47.260 | 25.934 | 41.174 | 1.00 | 28.53 |
| 849 | CZ | ARG | 106 | 47.904 | 25.010 | 41.864 | 1.00 | 28.53 |
| 850 | NH1 | ARG | 106 | 47.633 | 24.925 | 43.173 | 1.00 | 28.53 |
| 851 | NH2 | ARG | 106 | 48.819 | 24.231 | 41.267 | 1.00 | 28.53 |
| 852 | C | ARG | 106 | 42.775 | 28.496 | 40.663 | 1.00 | 60.02 |
| 853 | O | ARG | 106 | 42.121 | 27.884 | 41.498 | 1.00 | 60.02 |
| 854 | N | CYS | 107 | 42.625 | 28.378 | 39.353 | 1.00 | 30.47 |
| 855 | CA | CYS | 107 | 41.692 | 27.449 | 38.763 | 1.00 | 30.47 |
| 856 | C | CYS | 107 | 42.484 | 26.168 | 38.645 | 1.00 | 30.47 |
| 857 | O | CYS | 107 | 43.098 | 25.892 | 37.607 | 1.00 | 30.47 |
| 858 | CB | CYS | 107 | 41.261 | 27.937 | 37.398 | 1.00 | 52.55 |
| 859 | SG | CYS | 107 | 39.630 | 27.258 | 36.984 | 1.00 | 52.55 |
| 860 | N | HIS | 108 | 42.476 | 25.399 | 39.733 | 1.00 | 44.34 |
| 861 | CA | HIS | 108 | 43.224 | 24.162 | 39.808 | 1.00 | 44.34 |
| 862 | CB | HIS | 108 | 43.644 | 23.916 | 41.229 | 1.00 | 41.81 |
| 863 | CG | HIS | 108 | 44.610 | 22.800 | 41.351 | 1.00 | 41.81 |
| 864 | CD2 | HIS | 108 | 44.648 | 21.734 | 42.178 | 1.00 | 41.81 |
| 865 | ND1 | HIS | 108 | 45.701 | 22.686 | 40.519 | 1.00 | 41.81 |
| 866 | CE1 | HIS | 108 | 46.370 | 21.595 | 40.832 | 1.00 | 41.81 |
| 867 | NE2 | HIS | 108 | 45.753 | 20.999 | 41.835 | 1.00 | 41.81 |
| 868 | C | HIS | 108 | 42.526 | 22.934 | 39.310 | 1.00 | 44.34 |
| 869 | O | HIS | 108 | 41.515 | 22.530 | 39.838 | 1.00 | 44.34 |
| 870 | N | GLY | 109 | 43.084 | 22.315 | 38.293 | 1.00 | 45.07 |
| 871 | CA | GLY | 109 | 42.461 | 21.121 | 37.772 | 1.00 | 45.07 |
| 872 | C | GLY | 109 | 42.909 | 19.890 | 38.534 | 1.00 | 45.07 |
| 873 | O | GLY | 109 | 44.015 | 19.831 | 39.097 | 1.00 | 45.07 |
| 874 | N | TRP | 110 | 42.026 | 18.902 | 38.565 | 1.00 | 46.18 |
| 875 | CA | TRP | 110 | 42.293 | 17.641 | 39.239 | 1.00 | 46.18 |
| 876 | CB | TRP | 110 | 41.156 | 16.653 | 38.932 | 1.00 | 36.88 |
| 877 | CG | TRP | 110 | 41.347 | 15.329 | 39.576 | 1.00 | 36.88 |
| 878 | CD2 | TRP | 110 | 41.040 | 15.001 | 40.923 | 1.00 | 36.88 |
| 879 | CE2 | TRP | 110 | 41.526 | 13.697 | 41.176 | 1.00 | 36.88 |
| 880 | CE3 | TRP | 110 | 40.388 | 15.690 | 41.973 | 1.00 | 36.88 |
| 881 | CD1 | TRP | 110 | 41.989 | 14.231 | 39.047 | 1.00 | 36.88 |
| 882 | NE1 | TRP | 110 | 42.110 | 13.251 | 39.999 | 1.00 | 36.88 |
| 883 | CZ2 | TRP | 110 | 41.408 | 13.055 | 42.401 | 1.00 | 36.88 |
| 884 | CZ3 | TRP | 110 | 40.264 | 15.061 | 43.204 | 1.00 | 36.88 |
| 885 | CH2 | TRP | 110 | 40.773 | 13.748 | 43.408 | 1.00 | 36.88 |
| 886 | C | TRP | 110 | 43.615 | 17.100 | 38.726 | 1.00 | 46.18 |
| 887 | O | TRP | 110 | 43.910 | 17.235 | 37.539 | 1.00 | 46.18 |
| 888 | N | ARG | 111 | 44.416 | 16.510 | 39.609 | 1.00 | 63.08 |
| 889 | CA | ARG | 111 | 45.704 | 15.952 | 39.200 | 1.00 | 63.08 |
| 890 | CB | ARG | 111 | 45.488 | 14.690 | 38.373 | 1.00 | 97.96 |
| 891 | CG | ARG | 111 | 44.790 | 13.594 | 39.132 | 1.00 | 97.96 |
| 892 | CD | ARG | 111 | 45.652 | 13.108 | 40.265 | 1.00 | 97.96 |
| 893 | NE | ARG | 111 | 46.850 | 12.447 | 39.758 | 1.00 | 97.96 |
| 894 | CZ | ARG | 111 | 47.845 | 12.019 | 40.528 | 1.00 | 97.96 |
| 895 | NH1 | ARG | 111 | 47.785 | 12.190 | 41.846 | 1.00 | 97.96 |
| 896 | NH2 | ARG | 111 | 48.894 | 11.413 | 39.982 | 1.00 | 97.96 |
| 897 | C | ARG | 111 | 46.465 | 16.961 | 38.358 | 1.00 | 63.08 |
| 898 | O | ARG | 111 | 47.152 | 16.596 | 37.406 | 1.00 | 63.08 |
| 899 | N | ASN | 112 | 46.324 | 18.237 | 38.685 | 1.00 | 111.35 |
| 900 | CA | ASN | 112 | 47.011 | 19.247 | 37.914 | 1.00 | 111.35 |
| 901 | CB | ASN | 112 | 48.500 | 19.213 | 38.245 | 1.00 | 81.63 |
| 902 | CG | ASN | 112 | 48.814 | 19.880 | 39.568 | 1.00 | 81.63 |
| 903 | OD1 | ASN | 112 | 48.719 | 21.101 | 39.701 | 1.00 | 81.63 |
| 904 | ND2 | ASN | 112 | 49.182 | 19.082 | 40.556 | 1.00 | 81.63 |
| 905 | C | ASN | 112 | 46.791 | 18.987 | 36.424 | 1.00 | 111.35 |
| 906 | O | ASN | 112 | 47.744 | 18.946 | 35.648 | 1.00 | 111.35 |
| 907 | N | TRP | 113 | 45.540 | 18.783 | 36.027 | 1.00 | 91.49 |
| 908 | CA | TRP | 113 | 45.258 | 18.560 | 34.623 | 1.00 | 91.49 |
| 909 | CB | TRP | 113 | 43.894 | 17.929 | 34.393 | 1.00 | 88.74 |
| 910 | CG | TRP | 113 | 43.813 | 16.506 | 34.706 | 1.00 | 88.74 |
| 911 | CD2 | TRP | 113 | 42.611 | 15.767 | 34.927 | 1.00 | 88.74 |
| 912 | CE2 | TRP | 113 | 42.998 | 14.439 | 35.200 | 1.00 | 88.74 |
| 913 | CE3 | TRP | 113 | 41.258 | 16.105 | 34.927 | 1.00 | 88.74 |
| 914 | CD1 | TRP | 113 | 44.845 | 15.625 | 34.837 | 1.00 | 88.74 |
| 915 | NE1 | TRP | 113 | 44.354 | 14.376 | 35.137 | 1.00 | 88.74 |
| 916 | CZ2 | TRP | 113 | 42.063 | 13.451 | 35.465 | 1.00 | 88.74 |
| 917 | CZ3 | TRP | 113 | 40.338 | 15.124 | 35.188 | 1.00 | 88.74 |
| 918 | CH2 | TRP | 113 | 40.742 | 13.810 | 35.459 | 1.00 | 88.74 |
| 919 | C | TRP | 113 | 45.255 | 19.871 | 33.895 | 1.00 | 91.49 |
| 920 | O | TRP | 113 | 44.941 | 20.922 | 34.463 | 1.00 | 91.49 |
| 921 | N | ASP | 114 | 45.567 | 19.780 | 32.611 | 1.00 | 60.02 |
| 922 | CA | ASP | 114 | 45.599 | 20.940 | 31.755 | 1.00 | 60.02 |
| 923 | CB | ASP | 114 | 46.201 | 20.569 | 30.390 | 1.00 | 89.00 |
| 924 | CG | ASP | 114 | 47.637 | 20.066 | 30.498 | 1.00 | 89.00 |
| 925 | OD1 | ASP | 114 | 48.360 | 20.492 | 31.430 | 1.00 | 89.00 |
| 926 | OD2 | ASP | 114 | 48.053 | 19.254 | 29.643 | 1.00 | 89.00 |
| 927 | C | ASP | 114 | 44.210 | 21.555 | 31.585 | 1.00 | 60.02 |
| 928 | O | ASP | 114 | 43.248 | 20.897 | 31.224 | 1.00 | 60.02 |
| 929 | N | VAL | 115 | 44.104 | 22.832 | 31.879 | 1.00 | 103.64 |
| 930 | CA | VAL | 115 | 42.836 | 23.482 | 31.712 | 1.00 | 103.64 |
| 931 | CB | VAL | 115 | 42.283 | 23.916 | 33.063 | 1.00 | 73.31 |
| 932 | CG1 | VAL | 115 | 40.951 | 24.620 | 32.902 | 1.00 | 73.31 |
| 933 | CG2 | VAL | 115 | 42.153 | 22.682 | 33.943 | 1.00 | 73.31 |
| 934 | C | VAL | 115 | 43.127 | 24.647 | 30.805 | 1.00 | 103.64 |
| 935 | O | VAL | 115 | 44.162 | 25.304 | 30.904 | 1.00 | 103.64 |
| 936 | N | TYR | 116 | 42.232 | 24.861 | 29.868 | 1.00 | 59.95 |
| 937 | CA | TYR | 116 | 42.415 | 25.932 | 28.943 | 1.00 | 59.95 |
| 938 | CB | TYR | 116 | 42.503 | 25.377 | 27.529 | 1.00 | 98.72 |
| 939 | CG | TYR | 116 | 43.712 | 24.492 | 27.362 | 1.00 | 98.72 |
| 940 | CD1 | TYR | 116 | 43.675 | 23.152 | 27.726 | 1.00 | 98.72 |
| 941 | CE1 | TYR | 116 | 44.815 | 22.360 | 27.650 | 1.00 | 98.72 |
| 942 | CD2 | TYR | 116 | 44.922 | 25.017 | 26.914 | 1.00 | 98.72 |
| 943 | CE2 | TYR | 116 | 46.067 | 24.233 | 26.839 | 1.00 | 98.72 |
| 944 | CZ | TYR | 116 | 46.008 | 22.908 | 27.207 | 1.00 | 98.72 |
| 945 | OH | TYR | 116 | 47.147 | 22.132 | 27.144 | 1.00 | 98.72 |
| 946 | C | TYR | 116 | 41.271 | 26.898 | 29.083 | 1.00 | 59.95 |
| 947 | O | TYR | 116 | 40.499 | 26.821 | 30.047 | 1.00 | 59.95 |
| 948 | N | LYS | 117 | 41.176 | 27.814 | 28.127 | 1.00 | 66.79 |
| 949 | CA | LYS | 117 | 40.132 | 28.813 | 28.132 | 1.00 | 66.79 |
| 950 | CB | LYS | 117 | 38.946 | 28.332 | 27.295 | 1.00 | 151.68 |
| 951 | CG | LYS | 117 | 37.902 | 29.406 | 27.008 | 1.00 | 151.68 |
| 952 | CD | LYS | 117 | 38.475 | 30.515 | 26.129 | 1.00 | 151.68 |
| 953 | CE | LYS | 117 | 37.451 | 31.604 | 25.848 | 1.00 | 151.68 |
| 954 | NZ | LYS | 117 | 38.062 | 32.685 | 25.034 | 1.00 | 151.68 |
| 955 | C | LYS | 117 | 39.676 | 29.122 | 29.559 | 1.00 | 66.79 |
| 956 | O | LYS | 117 | 38.472 | 29.233 | 29.810 | 1.00 | 66.79 |
| 957 | N | VAL | 118 | 40.622 | 29.256 | 30.495 | 1.00 | 43.61 |
| 958 | CA | VAL | 118 | 40.243 | 29.564 | 31.879 | 1.00 | 43.61 |
| 959 | CB | VAL | 118 | 41.429 | 29.461 | 32.852 | 1.00 | 34.30 |
| 960 | CG1 | VAL | 118 | 40.912 | 29.485 | 34.289 | 1.00 | 34.30 |
| 961 | CG2 | VAL | 118 | 42.246 | 28.215 | 32.562 | 1.00 | 34.30 |
| 962 | C | VAL | 118 | 39.657 | 30.977 | 31.998 | 1.00 | 43.61 |
| 963 | O | VAL | 118 | 40.002 | 31.881 | 31.235 | 1.00 | 43.61 |
| 964 | N | ILE | 119 | 38.772 | 31.168 | 32.963 | 1.00 | 47.57 |
| 965 | CA | ILE | 119 | 38.135 | 32.458 | 33.140 | 1.00 | 47.57 |
| 966 | CB | ILE | 119 | 36.895 | 32.621 | 32.203 | 1.00 | 48.87 |
| 967 | CG2 | ILE | 119 | 36.185 | 33.914 | 32.512 | 1.00 | 48.87 |
| 968 | CG1 | ILE | 119 | 37.317 | 32.657 | 30.730 | 1.00 | 48.87 |
| 969 | CD1 | ILE | 119 | 36.187 | 33.009 | 29.752 | 1.00 | 48.87 |
| 970 | C | ILE | 119 | 37.651 | 32.620 | 34.567 | 1.00 | 47.57 |
| 971 | O | ILE | 119 | 36.846 | 31.827 | 35.052 | 1.00 | 47.57 |
| 972 | N | TYR | 120 | 38.125 | 33.645 | 35.256 | 1.00 | 51.27 |
| 973 | CA | TYR | 120 | 37.647 | 33.835 | 36.608 | 1.00 | 51.27 |
| 974 | CB | TYR | 120 | 38.727 | 34.342 | 37.510 | 1.00 | 24.86 |
| 975 | CG | TYR | 120 | 39.921 | 33.471 | 37.546 | 1.00 | 24.86 |
| 976 | CD1 | TYR | 120 | 40.623 | 33.174 | 36.393 | 1.00 | 24.86 |
| 977 | CE1 | TYR | 120 | 41.794 | 32.429 | 36.448 | 1.00 | 24.86 |
| 978 | CD2 | TYR | 120 | 40.397 | 33.004 | 38.754 | 1.00 | 24.86 |
| 979 | CE2 | TYR | 120 | 41.562 | 32.258 | 38.832 | 1.00 | 24.86 |
| 980 | CZ | TYR | 120 | 42.260 | 31.972 | 37.681 | 1.00 | 24.86 |
| 981 | OH | TYR | 120 | 43.423 | 31.233 | 37.816 | 1.00 | 24.86 |
| 982 | C | TYR | 120 | 36.531 | 34.832 | 36.621 | 1.00 | 51.27 |
| 983 | O | TYR | 120 | 36.486 | 35.751 | 35.802 | 1.00 | 51.27 |
| 984 | N | TYR | 121 | 35.645 | 34.659 | 37.584 | 1.00 | 57.39 |
| 985 | CA | TYR | 121 | 34.506 | 35.530 | 37.701 | 1.00 | 57.39 |
| 986 | CB | TYR | 121 | 33.215 | 34.815 | 37.266 | 1.00 | 81.52 |
| 987 | CG | TYR | 121 | 33.180 | 34.361 | 35.836 | 1.00 | 81.52 |
| 988 | CD1 | TYR | 121 | 33.923 | 33.263 | 35.423 | 1.00 | 81.52 |
| 989 | CE1 | TYR | 121 | 33.918 | 32.846 | 34.105 | 1.00 | 81.52 |
| 990 | CD2 | TYR | 121 | 32.419 | 35.042 | 34.889 | 1.00 | 81.52 |
| 991 | CE2 | TYR | 121 | 32.407 | 34.642 | 33.554 | 1.00 | 81.52 |
| 992 | CZ | TYR | 121 | 33.163 | 33.537 | 33.171 | 1.00 | 81.52 |
| 993 | OH | TYR | 121 | 33.195 | 33.117 | 31.859 | 1.00 | 81.52 |
| 994 | C | TYR | 121 | 34.315 | 35.966 | 39.124 | 1.00 | 57.39 |
| 995 | O | TYR | 121 | 34.522 | 35.184 | 40.054 | 1.00 | 57.39 |
| 996 | N | LYS | 122 | 33.907 | 37.218 | 39.282 | 1.00 | 81.15 |
| 997 | CA | LYS | 122 | 33.595 | 37.748 | 40.589 | 1.00 | 81.15 |
| 998 | CB | LYS | 122 | 34.433 | 38.961 | 40.948 | 1.00 | 110.18 |
| 999 | CG | LYS | 122 | 34.253 | 39.334 | 42.403 | 1.00 | 110.18 |
| 1000 | CD | LYS | 122 | 34.827 | 40.684 | 42.722 | 1.00 | 110.18 |
| 1001 | CE | LYS | 122 | 33.834 | 41.772 | 42.392 | 1.00 | 110.18 |
| 1002 | NZ | LYS | 122 | 34.358 | 43.095 | 42.796 | 1.00 | 110.18 |
| 1003 | C | LYS | 122 | 32.159 | 38.177 | 40.444 | 1.00 | 81.15 |
| 1004 | O | LYS | 122 | 31.834 | 39.050 | 39.642 | 1.00 | 81.15 |
| 1005 | N | ASP | 123 | 31.289 | 37.539 | 41.209 | 1.00 | 100.05 |
| 1006 | CA | ASP | 123 | 29.875 | 37.861 | 41.159 | 1.00 | 100.05 |
| 1007 | CB | ASP | 123 | 29.640 | 39.238 | 41.781 | 1.00 | 124.27 |
| 1008 | CG | ASP | 123 | 30.336 | 39.380 | 43.124 | 1.00 | 124.27 |
| 1009 | OD1 | ASP | 123 | 30.145 | 38.499 | 43.991 | 1.00 | 124.27 |
| 1010 | OD2 | ASP | 123 | 31.075 | 40.370 | 43.309 | 1.00 | 124.27 |
| 1011 | C | ASP | 123 | 29.401 | 37.816 | 39.720 | 1.00 | 100.05 |
| 1012 | O | ASP | 123 | 29.086 | 38.844 | 39.125 | 1.00 | 100.05 |
| 1013 | N | GLY | 124 | 29.407 | 36.612 | 39.157 | 1.00 | 103.70 |
| 1014 | CA | GLY | 124 | 28.929 | 36.423 | 37.803 | 1.00 | 103.70 |
| 1015 | C | GLY | 124 | 29.750 | 37.084 | 36.729 | 1.00 | 103.70 |
| 1016 | O | GLY | 124 | 29.983 | 36.517 | 35.684 | 1.00 | 103.70 |
| 1017 | N | GLU | 125 | 30.231 | 38.285 | 37.011 | 1.00 | 86.80 |
| 1018 | CA | GLU | 125 | 31.033 | 39.025 | 36.046 | 1.00 | 86.80 |
| 1019 | CB | GLU | 125 | 30.929 | 40.506 | 36.309 | 1.00 | 167.98 |
| 1020 | CG | GLU | 125 | 29.523 | 41.030 | 36.184 | 1.00 | 167.98 |
| 1021 | CD | GLU | 125 | 29.407 | 42.485 | 36.603 | 1.00 | 167.98 |
| 1022 | OE1 | GLU | 125 | 30.209 | 43.326 | 36.133 | 1.00 | 167.98 |
| 1023 | OE2 | GLU | 125 | 28.505 | 42.791 | 37.413 | 1.00 | 167.98 |
| 1024 | C | GLU | 125 | 32.515 | 38.651 | 35.940 | 1.00 | 86.80 |
| 1025 | O | GLU | 125 | 33.206 | 38.440 | 36.952 | 1.00 | 86.80 |
| 1026 | N | ALA | 126 | 33.003 | 38.614 | 34.700 | 1.00 | 117.82 |
| 1027 | CA | ALA | 126 | 34.395 | 38.279 | 34.411 | 1.00 | 117.82 |
| 1028 | CB | ALA | 126 | 34.627 | 38.310 | 32.891 | 1.00 | 16.51 |
| 1029 | C | ALA | 126 | 35.399 | 39.201 | 35.101 | 1.00 | 117.82 |
| 1030 | O | ALA | 126 | 35.141 | 40.384 | 35.284 | 1.00 | 117.82 |
| 1031 | N | LEU | 127 | 36.556 | 38.665 | 35.467 | 1.00 | 95.95 |
| 1032 | CA | LEU | 127 | 37.572 | 39.490 | 36.104 | 1.00 | 95.95 |
| 1033 | CB | LEU | 127 | 38.604 | 38.639 | 36.840 | 1.00 | 46.01 |
| 1034 | CG | LEU | 127 | 38.205 | 37.916 | 38.119 | 1.00 | 46.01 |
| 1035 | CD1 | LEU | 127 | 39.484 | 37.355 | 38.754 | 1.00 | 46.01 |
| 1036 | CD2 | LEU | 127 | 37.491 | 38.848 | 39.082 | 1.00 | 46.01 |
| 1037 | C | LEU | 127 | 38.281 | 40.327 | 35.050 | 1.00 | 95.95 |
| 1038 | O | LEU | 127 | 38.475 | 39.885 | 33.911 | 1.00 | 95.95 |
| 1039 | N | LYS | 128 | 38.664 | 41.538 | 35.450 | 1.00 | 158.42 |
| 1040 | CA | LYS | 128 | 39.342 | 42.486 | 34.573 | 1.00 | 158.42 |
| 1041 | CB | LYS | 128 | 39.220 | 43.899 | 35.142 | 1.00 | 155.76 |
| 1042 | CG | LYS | 128 | 37.787 | 44.386 | 35.312 | 1.00 | 155.76 |
| 1043 | CD | LYS | 128 | 37.114 | 44.590 | 33.960 | 1.00 | 155.76 |
| 1044 | CE | LYS | 128 | 35.689 | 45.116 | 34.097 | 1.00 | 155.76 |
| 1045 | NZ | LYS | 128 | 35.067 | 45.392 | 32.766 | 1.00 | 155.76 |
| 1046 | C | LYS | 128 | 40.813 | 42.148 | 34.369 | 1.00 | 158.42 |
| 1047 | O | LYS | 128 | 41.144 | 41.178 | 33.693 | 1.00 | 158.42 |
| 1048 | N | TYR | 129 | 41.698 | 42.949 | 34.954 | 1.00 | 117.79 |
| 1049 | CA | TYR | 129 | 43.132 | 42.714 | 34.800 | 1.00 | 117.79 |
| 1050 | CB | TYR | 129 | 43.851 | 43.967 | 34.297 | 1.00 | 165.79 |
| 1051 | CG | TYR | 129 | 43.199 | 44.664 | 33.137 | 1.00 | 165.79 |
| 1052 | CD1 | TYR | 129 | 42.151 | 45.563 | 33.344 | 1.00 | 165.79 |
| 1053 | CE1 | TYR | 129 | 41.561 | 46.234 | 32.280 | 1.00 | 165.79 |
| 1054 | CD2 | TYR | 129 | 43.643 | 44.447 | 31.832 | 1.00 | 165.79 |
| 1055 | CE2 | TYR | 129 | 43.060 | 45.112 | 30.759 | 1.00 | 165.79 |
| 1056 | CZ | TYR | 129 | 42.022 | 46.005 | 30.991 | 1.00 | 165.79 |
| 1057 | OH | TYR | 129 | 41.451 | 46.671 | 29.932 | 1.00 | 165.79 |
| 1058 | C | TYR | 129 | 43.834 | 42.279 | 36.077 | 1.00 | 117.79 |
| 1059 | O | TYR | 129 | 44.261 | 43.117 | 36.875 | 1.00 | 117.79 |
| 1060 | N | TRP | 130 | 43.956 | 40.972 | 36.267 | 1.00 | 50.95 |
| 1061 | CA | TRP | 130 | 44.647 | 40.433 | 37.422 | 1.00 | 50.95 |
| 1062 | CB | TRP | 130 | 43.933 | 39.172 | 37.899 | 1.00 | 147.57 |
| 1063 | CG | TRP | 130 | 42.728 | 39.505 | 38.701 | 1.00 | 147.57 |
| 1064 | CD2 | TRP | 130 | 42.410 | 39.002 | 39.993 | 1.00 | 147.57 |
| 1065 | CE2 | TRP | 130 | 41.230 | 39.650 | 40.406 | 1.00 | 147.57 |
| 1066 | CE3 | TRP | 130 | 43.023 | 38.088 | 40.853 | 1.00 | 147.57 |
| 1067 | CD1 | TRP | 130 | 41.744 | 40.394 | 38.378 | 1.00 | 147.57 |
| 1068 | NE1 | TRP | 130 | 40.841 | 40.486 | 39.400 | 1.00 | 147.57 |
| 1069 | CZ2 | TRP | 130 | 40.636 | 39.388 | 41.632 | 1.00 | 147.57 |
| 1070 | CZ3 | TRP | 130 | 42.436 | 37.831 | 42.068 | 1.00 | 147.57 |
| 1071 | CH2 | TRP | 130 | 41.257 | 38.488 | 42.454 | 1.00 | 147.57 |
| 1072 | C | TRP | 130 | 46.011 | 40.129 | 36.846 | 1.00 | 50.95 |
| 1073 | O | TRP | 130 | 46.184 | 40.232 | 35.628 | 1.00 | 50.95 |
| 1074 | N | TYR | 131 | 46.980 | 39.759 | 37.670 | 1.00 | 143.97 |
| 1075 | CA | TYR | 131 | 48.287 | 39.493 | 37.101 | 1.00 | 143.97 |
| 1076 | CB | TYR | 131 | 49.320 | 39.136 | 38.170 | 1.00 | 125.03 |
| 1077 | CG | TYR | 131 | 50.636 | 38.736 | 37.544 | 1.00 | 125.03 |
| 1078 | CD1 | TYR | 131 | 51.129 | 39.426 | 36.443 | 1.00 | 125.03 |
| 1079 | CE1 | TYR | 131 | 52.276 | 39.026 | 35.794 | 1.00 | 125.03 |
| 1080 | CD2 | TYR | 131 | 51.347 | 37.634 | 37.995 | 1.00 | 125.03 |
| 1081 | CE2 | TYR | 131 | 52.511 | 37.230 | 37.352 | 1.00 | 125.03 |
| 1082 | CZ | TYR | 131 | 52.964 | 37.927 | 36.249 | 1.00 | 125.03 |
| 1083 | OH | TYR | 131 | 54.078 | 37.504 | 35.573 | 1.00 | 125.03 |
| 1084 | C | TYR | 131 | 48.285 | 38.404 | 36.033 | 1.00 | 143.97 |
| 1085 | O | TYR | 131 | 48.818 | 38.608 | 34.943 | 1.00 | 143.97 |
| 1086 | N | GLU | 132 | 47.700 | 37.254 | 36.351 | 1.00 | 105.73 |
| 1087 | CA | GLU | 132 | 47.628 | 36.119 | 35.427 | 1.00 | 105.73 |
| 1088 | CB | GLU | 132 | 47.113 | 36.560 | 34.053 | 1.00 | 172.31 |
| 1089 | CG | GLU | 132 | 46.992 | 35.425 | 33.034 | 1.00 | 172.31 |
| 1090 | CD | GLU | 132 | 46.449 | 35.896 | 31.695 | 1.00 | 172.31 |
| 1091 | OE1 | GLU | 132 | 45.321 | 36.436 | 31.670 | 1.00 | 172.31 |
| 1092 | OE2 | GLU | 132 | 47.148 | 35.724 | 30.670 | 1.00 | 172.31 |
| 1093 | C | GLU | 132 | 48.962 | 35.397 | 35.256 | 1.00 | 105.73 |
| 1094 | O | GLU | 132 | 50.022 | 35.935 | 35.552 | 1.00 | 105.73 |
| 1095 | N | ASN | 133 | 48.887 | 34.172 | 34.758 | 1.00 | 117.87 |
| 1096 | CA | ASN | 133 | 50.061 | 33.336 | 34.554 | 1.00 | 117.87 |
| 1097 | CB | ASN | 133 | 50.894 | 33.311 | 35.844 | 1.00 | 184.60 |
| 1098 | CG | ASN | 133 | 52.234 | 32.621 | 35.675 | 1.00 | 184.60 |
| 1099 | OD1 | ASN | 133 | 52.612 | 32.227 | 34.573 | 1.00 | 184.60 |
| 1100 | ND2 | ASN | 133 | 52.966 | 32.479 | 36.775 | 1.00 | 184.60 |
| 1101 | C | ASN | 133 | 49.470 | 31.961 | 34.285 | 1.00 | 117.87 |
| 1102 | O | ASN | 133 | 48.297 | 31.741 | 34.563 | 1.00 | 117.87 |
| 1103 | N | HIS | 134 | 50.248 | 31.038 | 33.732 | 1.00 | 156.61 |
| 1104 | CA | HIS | 134 | 49.714 | 29.700 | 33.509 | 1.00 | 156.61 |
| 1105 | CB | HIS | 134 | 50.697 | 28.835 | 32.710 | 1.00 | 161.49 |
| 1106 | CG | HIS | 134 | 50.137 | 27.491 | 32.325 | 1.00 | 161.49 |
| 1107 | CD2 | HIS | 134 | 50.423 | 26.249 | 32.787 | 1.00 | 161.49 |
| 1108 | ND1 | HIS | 134 | 49.130 | 27.348 | 31.409 | 1.00 | 161.49 |
| 1109 | CE1 | HIS | 134 | 48.802 | 26.061 | 31.313 | 1.00 | 161.49 |
| 1110 | NE2 | HIS | 134 | 49.569 | 25.382 | 32.136 | 1.00 | 161.49 |
| 1111 | C | HIS | 134 | 49.547 | 29.124 | 34.915 | 1.00 | 156.61 |
| 1112 | O | HIS | 134 | 49.172 | 27.967 | 35.093 | 1.00 | 156.61 |
| 1113 | N | ASN | 135 | 49.821 | 29.965 | 35.910 | 1.00 | 97.51 |
| 1114 | CA | ASN | 135 | 49.755 | 29.570 | 37.304 | 1.00 | 97.51 |
| 1115 | CB | ASN | 135 | 51.160 | 29.704 | 37.934 | 1.00 | 110.39 |
| 1116 | CG | ASN | 135 | 52.230 | 28.875 | 37.217 | 1.00 | 110.39 |
| 1117 | OD1 | ASN | 135 | 51.990 | 27.742 | 36.801 | 1.00 | 110.39 |
| 1118 | ND2 | ASN | 135 | 53.438 | 29.433 | 37.117 | 1.00 | 110.39 |
| 1119 | C | ASN | 135 | 48.729 | 30.282 | 38.193 | 1.00 | 97.51 |
| 1120 | O | ASN | 135 | 48.097 | 29.646 | 39.031 | 1.00 | 97.51 |
| 1121 | N | ILE | 136 | 48.542 | 31.589 | 38.037 | 1.00 | 101.73 |
| 1122 | CA | ILE | 136 | 47.600 | 32.267 | 38.943 | 1.00 | 101.73 |
| 1123 | CB | ILE | 136 | 48.307 | 32.728 | 40.212 | 1.00 | 31.25 |
| 1124 | CG2 | ILE | 136 | 47.296 | 33.031 | 41.311 | 1.00 | 31.25 |
| 1125 | CG1 | ILE | 136 | 49.240 | 31.649 | 40.728 | 1.00 | 31.25 |
| 1126 | CD1 | ILE | 136 | 49.753 | 31.893 | 42.146 | 1.00 | 31.25 |
| 1127 | C | ILE | 136 | 46.867 | 33.476 | 38.424 | 1.00 | 101.73 |
| 1128 | O | ILE | 136 | 46.274 | 33.477 | 37.354 | 1.00 | 101.73 |
| 1129 | N | SER | 137 | 46.901 | 34.498 | 39.257 | 1.00 | 57.41 |
| 1130 | CA | SER | 137 | 46.286 | 35.780 | 39.011 | 1.00 | 57.41 |
| 1131 | CB | SER | 137 | 44.901 | 35.617 | 38.380 | 1.00 | 67.48 |
| 1132 | CG | SER | 137 | 44.052 | 35.011 | 39.328 | 1.00 | 67.48 |
| 1133 | C | SER | 137 | 46.183 | 36.488 | 40.392 | 1.00 | 57.41 |
| 1134 | O | SER | 137 | 45.619 | 35.936 | 41.348 | 1.00 | 57.41 |
| 1135 | N | ILE | 138 | 46.730 | 37.708 | 40.476 | 1.00 | 65.35 |
| 1136 | CA | ILE | 138 | 46.751 | 38.515 | 41.708 | 1.00 | 65.35 |
| 1137 | CB | ILE | 138 | 48.186 | 38.693 | 42.184 | 1.00 | 90.30 |
| 1138 | CG2 | ILE | 138 | 48.232 | 39.050 | 43.659 | 1.00 | 90.30 |
| 1139 | CG1 | ILE | 138 | 48.941 | 37.407 | 41.930 | 1.00 | 90.30 |
| 1140 | CD1 | ILE | 138 | 50.416 | 37.583 | 41.970 | 1.00 | 90.30 |
| 1141 | C | ILE | 138 | 46.148 | 39.928 | 41.561 | 1.00 | 65.35 |
| 1142 | O | ILE | 138 | 46.060 | 40.471 | 40.456 | 1.00 | 65.35 |
| 1143 | N | THR | 139 | 45.745 | 40.515 | 42.683 | 1.00 | 86.56 |
| 1144 | CA | THR | 139 | 45.164 | 41.843 | 42.672 | 1.00 | 86.56 |
| 1145 | CB | THR | 139 | 43.649 | 41.817 | 42.377 | 1.00 | 127.73 |
| 1146 | OG1 | THR | 139 | 43.399 | 41.192 | 41.115 | 1.00 | 127.73 |
| 1147 | CG2 | THR | 139 | 43.093 | 43.253 | 42.386 | 1.00 | 127.73 |
| 1148 | C | THR | 139 | 45.331 | 42.466 | 44.043 | 1.00 | 86.56 |
| 1149 | O | THR | 139 | 45.198 | 41.779 | 45.051 | 1.00 | 86.56 |
| 1150 | N | ASN | 140 | 45.624 | 43.763 | 44.089 | 1.00 | 96.52 |
| 1151 | CA | ASN | 140 | 45.753 | 44.415 | 45.370 | 1.00 | 96.52 |
| 1152 | CB | ASN | 140 | 46.301 | 45.834 | 45.214 | 1.00 | 124.36 |
| 1153 | CG | ASN | 140 | 46.721 | 46.427 | 46.534 | 1.00 | 124.36 |
| 1154 | OD1 | ASN | 140 | 46.321 | 45.925 | 47.576 | 1.00 | 124.36 |
| 1155 | ND2 | ASN | 140 | 47.512 | 47.492 | 46.515 | 1.00 | 124.36 |
| 1156 | C | ASN | 140 | 44.325 | 44.432 | 45.916 | 1.00 | 96.52 |
| 1157 | O | ASN | 140 | 43.390 | 44.861 | 45.244 | 1.00 | 96.52 |
| 1158 | N | ALA | 141 | 44.168 | 43.921 | 47.126 | 1.00 | 84.53 |
| 1159 | CA | ALA | 141 | 42.876 | 43.846 | 47.769 | 1.00 | 84.53 |
| 1160 | CB | ALA | 141 | 43.022 | 43.170 | 49.104 | 1.00 | 49.12 |
| 1161 | C | ALA | 141 | 42.197 | 45.188 | 47.942 | 1.00 | 84.53 |
| 1162 | O | ALA | 141 | 42.778 | 46.143 | 48.459 | 1.00 | 84.53 |
| 1163 | N | THR | 142 | 40.942 | 45.223 | 47.514 | 1.00 | 86.11 |
| 1164 | CA | THR | 142 | 40.094 | 46.401 | 47.593 | 1.00 | 86.11 |
| 1165 | CB | THR | 142 | 39.660 | 46.860 | 46.184 | 1.00 | 85.26 |
| 1166 | OG1 | THR | 142 | 40.816 | 47.143 | 45.386 | 1.00 | 85.26 |
| 1167 | CG2 | THR | 142 | 38.793 | 48.102 | 46.268 | 1.00 | 85.26 |
| 1168 | C | THR | 142 | 38.847 | 45.955 | 48.337 | 1.00 | 86.11 |
| 1169 | O | THR | 142 | 38.577 | 44.766 | 48.411 | 1.00 | 86.11 |
| 1170 | N | VAL | 143 | 38.089 | 46.889 | 48.897 | 1.00 | 105.48 |
| 1171 | CA | VAL | 143 | 36.863 | 46.494 | 49.566 | 1.00 | 105.48 |
| 1172 | CB | VAL | 143 | 36.240 | 47.653 | 50.360 | 1.00 | 154.19 |
| 1173 | CG1 | VAL | 143 | 35.816 | 48.766 | 49.413 | 1.00 | 154.19 |
| 1174 | CG2 | VAL | 143 | 35.055 | 47.144 | 51.159 | 1.00 | 154.19 |
| 1175 | C | VAL | 143 | 35.917 | 46.092 | 48.428 | 1.00 | 105.48 |
| 1176 | O | VAL | 143 | 34.937 | 45.371 | 48.635 | 1.00 | 105.48 |
| 1177 | N | GLU | 144 | 36.238 | 46.560 | 47.223 | 1.00 | 97.40 |
| 1178 | CA | GLU | 144 | 35.454 | 46.269 | 46.022 | 1.00 | 97.40 |
| 1179 | CB | GLU | 144 | 35.815 | 47.241 | 44.905 | 1.00 | 151.67 |
| 1180 | CG | GLU | 144 | 35.304 | 48.645 | 45.100 | 1.00 | 151.67 |
| 1181 | CD | GLU | 144 | 35.772 | 49.574 | 44.000 | 1.00 | 151.67 |
| 1182 | OE1 | GLU | 144 | 35.580 | 49.236 | 42.811 | 1.00 | 151.67 |
| 1183 | OE2 | GLU | 144 | 36.330 | 50.643 | 44.324 | 1.00 | 151.67 |
| 1184 | C | GLU | 144 | 35.682 | 44.857 | 45.517 | 1.00 | 97.40 |
| 1185 | O | GLU | 144 | 34.784 | 44.232 | 44.963 | 1.00 | 97.40 |
| 1186 | N | ASP | 145 | 36.905 | 44.376 | 45.690 | 1.00 | 99.27 |
| 1187 | CA | ASP | 145 | 37.266 | 43.040 | 45.268 | 1.00 | 99.27 |
| 1188 | CB | ASP | 145 | 38.759 | 42.808 | 45.491 | 1.00 | 103.93 |
| 1189 | CG | ASP | 145 | 39.616 | 43.652 | 44.576 | 1.00 | 103.93 |
| 1190 | OD1 | ASP | 145 | 39.333 | 43.649 | 43.359 | 1.00 | 103.93 |
| 1191 | OD2 | ASP | 145 | 40.568 | 44.306 | 45.064 | 1.00 | 103.93 |
| 1192 | C | ASP | 145 | 36.461 | 42.016 | 46.042 | 1.00 | 99.27 |
| 1193 | O | ASP | 145 | 36.442 | 40.847 | 45.687 | 1.00 | 99.27 |
| 1194 | N | SER | 146 | 35.793 | 42.455 | 47.104 | 1.00 | 71.52 |
| 1195 | CA | SER | 146 | 34.985 | 41.545 | 47.900 | 1.00 | 71.52 |
| 1196 | CB | SER | 146 | 34.386 | 42.266 | 49.105 | 1.00 | 85.74 |
| 1197 | OG | SER | 146 | 35.371 | 42.424 | 50.111 | 1.00 | 85.74 |
| 1198 | C | SER | 146 | 33.900 | 40.948 | 47.012 | 1.00 | 71.52 |
| 1199 | O | SER | 146 | 33.469 | 41.569 | 46.036 | 1.00 | 71.52 |
| 1200 | N | GLY | 147 | 33.493 | 39.724 | 47.333 | 1.00 | 96.87 |
| 1201 | CA | GLY | 147 | 32.481 | 39.051 | 46.546 | 1.00 | 96.87 |
| 1202 | C | GLY | 147 | 32.739 | 37.567 | 46.394 | 1.00 | 96.87 |
| 1203 | O | GLY | 147 | 33.695 | 37.032 | 46.955 | 1.00 | 96.87 |
| 1204 | N | THR | 148 | 31.883 | 36.908 | 45.617 | 1.00 | 103.30 |
| 1205 | CA | THR | 148 | 31.975 | 35.469 | 45.377 | 1.00 | 103.30 |
| 1206 | CB | THR | 148 | 30.596 | 34.819 | 45.355 | 1.00 | 64.66 |
| 1207 | OG1 | THR | 148 | 30.231 | 34.568 | 43.990 | 1.00 | 64.66 |
| 1208 | CG2 | THR | 148 | 29.559 | 35.746 | 45.982 | 1.00 | 64.66 |
| 1209 | C | THR | 148 | 32.629 | 35.167 | 44.035 | 1.00 | 103.30 |
| 1210 | O | THR | 148 | 32.261 | 35.726 | 42.998 | 1.00 | 103.30 |
| 1211 | N | TYR | 149 | 33.586 | 34.253 | 44.059 | 1.00 | 77.50 |
| 1212 | CA | TYR | 149 | 34.294 | 33.888 | 42.853 | 1.00 | 77.50 |
| 1213 | CB | TYR | 149 | 35.798 | 34.053 | 43.059 | 1.00 | 78.57 |
| 1214 | CG | TYR | 149 | 36.237 | 35.491 | 43.209 | 1.00 | 78.57 |
| 1215 | CD1 | TYR | 149 | 35.965 | 36.210 | 44.372 | 1.00 | 78.57 |
| 1216 | CE1 | TYR | 149 | 36.349 | 37.540 | 44.492 | 1.00 | 78.57 |
| 1217 | CD2 | TYR | 149 | 36.907 | 36.141 | 42.169 | 1.00 | 78.57 |
| 1218 | CE2 | TYR | 149 | 37.291 | 37.463 | 42.277 | 1.00 | 78.57 |
| 1219 | CZ | TYR | 149 | 37.012 | 38.161 | 43.437 | 1.00 | 78.57 |
| 1220 | OH | TYR | 149 | 37.388 | 39.485 | 43.527 | 1.00 | 78.57 |
| 1221 | C | TYR | 149 | 34.018 | 32.469 | 42.439 | 1.00 | 77.50 |
| 1222 | O | TYR | 149 | 33.424 | 31.694 | 43.185 | 1.00 | 77.50 |
| 1223 | N | TYR | 150 | 34.473 | 32.150 | 41.232 | 1.00 | 44.44 |
| 1224 | CA | TYR | 150 | 34.335 | 30.823 | 40.648 | 1.00 | 44.44 |
| 1225 | CB | TYR | 150 | 32.851 | 30.446 | 40.588 | 1.00 | 66.36 |
| 1226 | CG | TYR | 150 | 32.132 | 30.968 | 39.376 | 1.00 | 66.36 |
| 1227 | CD1 | TYR | 150 | 32.205 | 30.288 | 38.157 | 1.00 | 66.36 |
| 1228 | CE1 | TYR | 150 | 31.585 | 30.776 | 37.034 | 1.00 | 66.36 |
| 1229 | CD2 | TYR | 150 | 31.411 | 32.156 | 39.433 | 1.00 | 66.36 |
| 1230 | CE2 | TYR | 150 | 30.788 | 32.655 | 38.315 | 1.00 | 66.36 |
| 1231 | CZ | TYR | 150 | 30.878 | 31.959 | 37.120 | 1.00 | 66.36 |
| 1232 | OH | TYR | 150 | 30.243 | 32.436 | 36.007 | 1.00 | 66.36 |
| 1233 | C | TYR | 150 | 34.957 | 30.881 | 39.240 | 1.00 | 44.44 |
| 1234 | O | TYR | 150 | 34.796 | 31.872 | 38.536 | 1.00 | 44.44 |
| 1235 | N | CYS | 151 | 35.677 | 29.842 | 38.833 | 1.00 | 64.10 |
| 1236 | CA | CYS | 151 | 36.290 | 29.853 | 37.513 | 1.00 | 64.10 |
| 1237 | C | CYS | 151 | 35.713 | 28.792 | 36.616 | 1.00 | 64.10 |
| 1238 | O | CYS | 151 | 35.015 | 27.897 | 37.067 | 1.00 | 64.10 |
| 1239 | CB | CYS | 151 | 37.813 | 29.668 | 37.612 | 1.00 | 75.24 |
| 1240 | SG | CYS | 151 | 38.407 | 28.123 | 38.380 | 1.00 | 75.24 |
| 1241 | N | THR | 152 | 36.021 | 28.904 | 35.334 | 1.00 | 63.66 |
| 1242 | CA | THR | 152 | 35.553 | 27.971 | 34.330 | 1.00 | 63.66 |
| 1243 | CB | THR | 152 | 34.453 | 28.599 | 33.459 | 1.00 | 48.15 |
| 1244 | OG1 | THR | 152 | 35.025 | 29.615 | 32.619 | 1.00 | 48.15 |
| 1245 | CG2 | THR | 152 | 33.360 | 29.204 | 34.341 | 1.00 | 48.15 |
| 1246 | C | THR | 152 | 36.779 | 27.708 | 33.479 | 1.00 | 63.66 |
| 1247 | O | THR | 152 | 37.709 | 28.512 | 33.473 | 1.00 | 63.66 |
| 1248 | N | GLY | 153 | 36.786 | 26.586 | 32.770 | 1.00 | 68.75 |
| 1249 | CA | GLY | 153 | 37.917 | 26.256 | 31.929 | 1.00 | 68.75 |
| 1250 | C | GLY | 153 | 37.588 | 24.988 | 31.180 | 1.00 | 68.75 |
| 1251 | O | GLY | 153 | 36.783 | 24.186 | 31.649 | 1.00 | 68.75 |
| 1252 | N | LYS | 154 | 38.192 | 24.800 | 30.016 | 1.00 | 56.66 |
| 1253 | CA | LYS | 154 | 37.936 | 23.603 | 29.248 | 1.00 | 56.66 |
| 1254 | CB | LYS | 154 | 37.984 | 23.917 | 27.751 | 1.00 | 131.33 |
| 1255 | CG | LYS | 154 | 37.756 | 22.699 | 26.874 | 1.00 | 131.33 |
| 1256 | CD | LYS | 154 | 38.014 | 22.985 | 25.401 | 1.00 | 131.33 |
| 1257 | CE | LYS | 154 | 37.989 | 21.684 | 24.596 | 1.00 | 131.33 |
| 1258 | NZ | LYS | 154 | 38.322 | 21.823 | 23.146 | 1.00 | 131.33 |
| 1259 | C | LYS | 154 | 38.932 | 22.501 | 29.582 | 1.00 | 56.66 |
| 1260 | O | LYS | 154 | 40.141 | 22.713 | 29.562 | 1.00 | 56.66 |
| 1261 | N | VAL | 155 | 38.412 | 21.321 | 29.913 | 1.00 | 93.07 |
| 1262 | CA | VAL | 155 | 39.260 | 20.162 | 30.203 | 1.00 | 93.07 |
| 1263 | CB | VAL | 155 | 38.924 | 19.490 | 31.544 | 1.00 | 78.48 |
| 1264 | CG1 | VAL | 155 | 40.072 | 18.590 | 31.955 | 1.00 | 78.48 |
| 1265 | CG2 | VAL | 155 | 38.659 | 20.536 | 32.606 | 1.00 | 78.48 |
| 1266 | C | VAL | 155 | 38.956 | 19.179 | 29.095 | 1.00 | 93.07 |
| 1267 | O | VAL | 155 | 37.802 | 19.005 | 28.719 | 1.00 | 93.07 |
| 1268 | N | TRP | 156 | 39.992 | 18.547 | 28.566 | 1.00 | 110.90 |
| 1269 | CA | TRP | 156 | 39.826 | 17.597 | 27.476 | 1.00 | 110.90 |
| 1270 | CB | TRP | 156 | 39.093 | 16.344 | 27.963 | 1.00 | 64.42 |
| 1271 | CG | TRP | 156 | 39.889 | 15.530 | 28.952 | 1.00 | 64.42 |
| 1272 | CD2 | TRP | 156 | 41.144 | 14.909 | 28.711 | 1.00 | 64.42 |
| 1273 | CE2 | TRP | 156 | 41.538 | 14.254 | 29.912 | 1.00 | 64.42 |
| 1274 | CE3 | TRP | 156 | 41.989 | 14.836 | 27.593 | 1.00 | 64.42 |
| 1275 | CD1 | TRP | 156 | 39.562 | 15.241 | 30.265 | 1.00 | 64.42 |
| 1276 | NE1 | TRP | 156 | 40.550 | 14.478 | 30.845 | 1.00 | 64.42 |
| 1277 | CZ2 | TRP | 156 | 42.732 | 13.535 | 30.029 | 1.00 | 64.42 |
| 1278 | CZ3 | TRP | 156 | 43.178 | 14.125 | 27.705 | 1.00 | 64.42 |
| 1279 | CH2 | TRP | 156 | 43.539 | 13.482 | 28.919 | 1.00 | 64.42 |
| 1280 | C | TRP | 156 | 39.063 | 18.269 | 26.335 | 1.00 | 110.90 |
| 1281 | O | TRP | 156 | 39.674 | 18.848 | 25.432 | 1.00 | 110.90 |
| 1282 | N | GLN | 157 | 37.738 | 18.225 | 26.366 | 1.00 | 82.18 |
| 1283 | CA | GLN | 157 | 36.980 | 18.857 | 25.298 | 1.00 | 82.18 |
| 1284 | CB | GLN | 157 | 36.566 | 17.802 | 24.260 | 1.00 | 143.76 |
| 1285 | CG | GLN | 157 | 37.656 | 16.778 | 23.860 | 1.00 | 143.76 |
| 1286 | CD | GLN | 157 | 37.147 | 15.761 | 22.835 | 1.00 | 143.76 |
| 1287 | OE1 | GLN | 157 | 35.954 | 15.470 | 22.790 | 1.00 | 143.76 |
| 1288 | NE2 | GLN | 157 | 38.050 | 15.211 | 22.025 | 1.00 | 143.76 |
| 1289 | C | GLN | 157 | 35.737 | 19.520 | 25.889 | 1.00 | 82.18 |
| 1290 | O | GLN | 157 | 34.910 | 20.068 | 25.166 | 1.00 | 82.18 |
| 1291 | N | LEU | 158 | 35.620 | 19.488 | 27.213 | 1.00 | 73.39 |
| 1292 | CA | LEU | 158 | 34.443 | 20.030 | 27.881 | 1.00 | 73.39 |
| 1293 | CB | LEU | 158 | 33.835 | 18.977 | 28.773 | 1.00 | 92.19 |
| 1294 | CG | LEU | 158 | 34.030 | 17.598 | 28.187 | 1.00 | 92.19 |
| 1295 | CD1 | LEU | 158 | 33.153 | 16.628 | 28.944 | 1.00 | 92.19 |
| 1296 | CD2 | LEU | 158 | 33.664 | 17.618 | 26.702 | 1.00 | 92.19 |
| 1297 | C | LEU | 158 | 34.658 | 21.254 | 28.724 | 1.00 | 73.39 |
| 1298 | O | LEU | 158 | 35.763 | 21.525 | 29.180 | 1.00 | 73.39 |
| 1299 | N | ASP | 159 | 33.564 | 21.955 | 28.992 | 1.00 | 43.69 |
| 1300 | CA | ASP | 159 | 33.615 | 23.183 | 29.761 | 1.00 | 43.69 |
| 1301 | CB | ASP | 159 | 32.773 | 24.249 | 29.074 | 1.00 | 86.10 |
| 1302 | CG | ASP | 159 | 33.085 | 24.373 | 27.594 | 1.00 | 86.10 |
| 1303 | OD1 | ASP | 159 | 34.289 | 24.477 | 27.249 | 1.00 | 86.10 |
| 1304 | OD2 | ASP | 159 | 32.128 | 24.374 | 26.778 | 1.00 | 86.10 |
| 1305 | C | ASP | 159 | 33.103 | 23.041 | 31.167 | 1.00 | 43.69 |
| 1306 | O | ASP | 159 | 31.900 | 22.907 | 31.357 | 1.00 | 43.69 |
| 1307 | N | TYR | 160 | 33.976 | 23.093 | 32.170 | 1.00 | 43.37 |
| 1308 | CA | TYR | 160 | 33.462 | 22.999 | 33.539 | 1.00 | 43.37 |
| 1309 | CB | TYR | 160 | 34.282 | 22.024 | 34.398 | 1.00 | 105.78 |
| 1310 | CG | TYR | 160 | 34.323 | 20.649 | 33.808 | 1.00 | 105.78 |
| 1311 | CD1 | TYR | 160 | 35.087 | 20.415 | 32.671 | 1.00 | 105.78 |
| 1312 | CE1 | TYR | 160 | 35.035 | 19.211 | 32.001 | 1.00 | 105.78 |
| 1313 | CD2 | TYR | 160 | 33.500 | 19.617 | 34.286 | 1.00 | 105.78 |
| 1314 | CE2 | TYR | 160 | 33.436 | 18.387 | 33.612 | 1.00 | 105.78 |
| 1315 | CZ | TYR | 160 | 34.209 | 18.205 | 32.458 | 1.00 | 105.78 |
| 1316 | OH | TYR | 160 | 34.143 | 17.068 | 31.692 | 1.00 | 105.78 |
| 1317 | C | TYR | 160 | 33.340 | 24.331 | 34.265 | 1.00 | 43.37 |
| 1318 | O | TYR | 160 | 33.620 | 25.407 | 33.736 | 1.00 | 43.37 |
| 1319 | N | GLU | 161 | 32.893 | 24.230 | 35.498 | 1.00 | 53.88 |
| 1320 | CA | GLU | 161 | 32.709 | 25.388 | 36.333 | 1.00 | 53.88 |
| 1321 | CB | GLU | 161 | 31.270 | 25.866 | 36.228 | 1.00 | 72.58 |
| 1322 | CG | GLU | 161 | 30.834 | 26.756 | 37.346 | 1.00 | 72.58 |
| 1323 | CD | GLU | 161 | 29.490 | 27.372 | 37.067 | 1.00 | 72.58 |
| 1324 | OE1 | GLU | 161 | 28.930 | 28.016 | 37.992 | 1.00 | 72.58 |
| 1325 | OE2 | GLU | 161 | 29.002 | 27.212 | 35.918 | 1.00 | 72.58 |
| 1326 | C | GLU | 161 | 33.002 | 24.902 | 37.725 | 1.00 | 53.88 |
| 1327 | O | GLU | 161 | 32.658 | 23.765 | 38.063 | 1.00 | 53.88 |
| 1328 | N | SER | 162 | 33.651 | 25.737 | 38.523 | 1.00 | 54.33 |
| 1329 | CA | SER | 162 | 33.967 | 25.330 | 39.868 | 1.00 | 54.33 |
| 1330 | CB | SER | 162 | 35.289 | 25.931 | 40.327 | 1.00 | 58.04 |
| 1331 | OG | SER | 162 | 35.183 | 27.339 | 40.452 | 1.00 | 58.04 |
| 1332 | C | SER | 162 | 32.868 | 25.781 | 40.794 | 1.00 | 54.33 |
| 1333 | O | SER | 162 | 31.975 | 26.508 | 40.391 | 1.00 | 54.33 |
| 1334 | N | GLU | 163 | 32.923 | 25.298 | 42.028 | 1.00 | 45.72 |
| 1335 | CA | GLU | 163 | 31.959 | 25.656 | 43.045 | 1.00 | 45.72 |
| 1336 | CB | GLU | 163 | 32.210 | 24.853 | 44.313 | 1.00 | 129.00 |
| 1337 | CG | GLU | 163 | 31.766 | 23.437 | 44.264 | 1.00 | 129.00 |
| 1338 | CD | GLU | 163 | 30.264 | 23.378 | 44.300 | 1.00 | 129.00 |
| 1339 | OE1 | GLU | 163 | 29.702 | 24.206 | 45.051 | 1.00 | 129.00 |
| 1340 | OE2 | GLU | 163 | 29.657 | 22.531 | 43.595 | 1.00 | 129.00 |
| 1341 | C | GLU | 163 | 32.259 | 27.102 | 43.356 | 1.00 | 45.72 |
| 1342 | O | GLU | 163 | 33.425 | 27.501 | 43.399 | 1.00 | 45.72 |
| 1343 | N | PRO | 164 | 31.219 | 27.914 | 43.576 | 1.00 | 67.32 |
| 1344 | CD | PRO | 164 | 29.759 | 27.742 | 43.567 | 1.00 | 86.92 |
| 1345 | CA | PRO | 164 | 31.578 | 29.289 | 43.884 | 1.00 | 67.32 |
| 1346 | CB | PRO | 164 | 30.227 | 29.992 | 43.934 | 1.00 | 86.92 |
| 1347 | CG | PRO | 164 | 29.312 | 28.921 | 44.383 | 1.00 | 86.92 |
| 1348 | C | PRO | 164 | 32.342 | 29.348 | 45.213 | 1.00 | 67.32 |
| 1349 | O | PRO | 164 | 32.402 | 28.372 | 45.973 | 1.00 | 67.32 |
| 1350 | N | LEU | 165 | 32.943 | 30.507 | 45.461 | 1.00 | 65.83 |
| 1351 | CA | LEU | 165 | 33.699 | 30.751 | 46.675 | 1.00 | 65.83 |
| 1352 | CB | LEU | 165 | 35.183 | 30.549 | 46.392 | 1.00 | 49.81 |
| 1353 | CG | LEU | 165 | 36.123 | 30.662 | 47.579 | 1.00 | 49.81 |
| 1354 | CD1 | LEU | 165 | 35.645 | 29.788 | 48.747 | 1.00 | 49.81 |
| 1355 | CD2 | LEU | 165 | 37.495 | 30.247 | 47.100 | 1.00 | 49.81 |
| 1356 | C | LEU | 165 | 33.424 | 32.177 | 47.156 | 1.00 | 65.83 |
| 1357 | O | LEU | 165 | 33.235 | 33.094 | 46.344 | 1.00 | 65.83 |
| 1358 | N | ASN | 166 | 33.376 | 32.358 | 48.473 | 1.00 | 106.87 |
| 1359 | CA | ASN | 166 | 33.117 | 33.676 | 49.033 | 1.00 | 106.87 |
| 1360 | CB | ASN | 166 | 31.975 | 33.597 | 50.055 | 1.00 | 138.19 |
| 1361 | CG | ASN | 166 | 30.601 | 33.541 | 49.380 | 1.00 | 138.19 |
| 1362 | OD1 | ASN | 166 | 30.053 | 32.448 | 49.150 | 1.00 | 138.19 |
| 1363 | ND2 | ASN | 166 | 30.075 | 34.724 | 49.034 | 1.00 | 138.19 |
| 1364 | C | ASN | 166 | 34.356 | 34.348 | 49.629 | 1.00 | 106.87 |
| 1365 | O | ASN | 166 | 34.960 | 33.853 | 50.578 | 1.00 | 106.87 |
| 1366 | N | ILE | 167 | 34.719 | 35.487 | 49.041 | 1.00 | 79.40 |
| 1367 | CA | ILE | 167 | 35.882 | 36.278 | 49.444 | 1.00 | 79.40 |
| 1368 | CB | ILE | 167 | 36.849 | 36.419 | 48.260 | 1.00 | 72.10 |
| 1369 | CG2 | ILE | 167 | 37.796 | 37.571 | 48.491 | 1.00 | 72.10 |
| 1370 | CG1 | ILE | 167 | 37.558 | 35.074 | 48.042 | 1.00 | 72.10 |
| 1371 | CD1 | ILE | 167 | 38.444 | 35.007 | 46.826 | 1.00 | 72.10 |
| 1372 | C | ILE | 167 | 35.502 | 37.662 | 49.951 | 1.00 | 79.40 |
| 1373 | O | ILE | 167 | 34.768 | 38.397 | 49.295 | 1.00 | 79.40 |
| 1374 | N | THR | 168 | 36.013 | 38.003 | 51.126 | 1.00 | 78.85 |
| 1375 | CA | THR | 168 | 35.727 | 39.284 | 51.750 | 1.00 | 78.85 |
| 1376 | CB | THR | 168 | 34.988 | 39.074 | 53.096 | 1.00 | 110.41 |
| 1377 | OG1 | THR | 168 | 33.724 | 38.444 | 52.850 | 1.00 | 110.41 |
| 1378 | CG2 | THR | 168 | 34.753 | 40.400 | 53.805 | 1.00 | 110.41 |
| 1379 | C | THR | 168 | 37.012 | 40.064 | 52.000 | 1.00 | 78.85 |
| 1380 | O | THR | 168 | 37.999 | 39.523 | 52.508 | 1.00 | 78.85 |
| 1381 | N | VAL | 169 | 36.997 | 41.338 | 51.634 | 1.00 | 91.78 |
| 1382 | CA | VAL | 169 | 38.163 | 42.177 | 51.829 | 1.00 | 91.78 |
| 1383 | CB | VAL | 169 | 38.475 | 42.968 | 50.576 | 1.00 | 74.43 |
| 1384 | CG1 | VAL | 169 | 39.886 | 43.524 | 50.663 | 1.00 | 74.43 |
| 1385 | CG2 | VAL | 169 | 38.303 | 42.079 | 49.361 | 1.00 | 74.43 |
| 1386 | C | VAL | 169 | 37.898 | 43.146 | 52.965 | 1.00 | 91.78 |
| 1387 | O | VAL | 169 | 37.524 | 44.297 | 52.738 | 1.00 | 91.78 |
| 1388 | N | ILE | 170 | 38.085 | 42.676 | 54.192 | 1.00 | 138.54 |
| 1389 | CA | ILE | 170 | 37.838 | 43.522 | 55.342 | 1.00 | 138.54 |
| 1390 | CB | ILE | 170 | 38.201 | 42.815 | 56.649 | 1.00 | 99.90 |
| 1391 | CG2 | ILE | 170 | 38.104 | 43.798 | 57.810 | 1.00 | 99.90 |
| 1392 | CG1 | ILE | 170 | 37.253 | 41.628 | 56.869 | 1.00 | 99.90 |
| 1393 | CD1 | ILE | 170 | 37.436 | 40.914 | 58.193 | 1.00 | 99.90 |
| 1394 | C | ILE | 170 | 38.598 | 44.837 | 55.255 | 1.00 | 138.54 |
| 1395 | O | ILE | 170 | 39.816 | 44.880 | 55.419 | 1.00 | 138.54 |
| 1396 | N | LYS | 171 | 37.856 | 45.912 | 55.002 | 1.00 | 166.26 |
| 1397 | CA | LYS | 171 | 38.420 | 47.254 | 54.878 | 1.00 | 166.26 |
| 1398 | CB | LYS | 171 | 37.322 | 48.228 | 54.430 | 1.00 | 153.43 |
| 1399 | CG | LYS | 171 | 37.806 | 49.608 | 53.987 | 1.00 | 153.43 |
| 1400 | CD | LYS | 171 | 36.637 | 50.449 | 53.489 | 1.00 | 153.43 |
| 1401 | CE | LYS | 171 | 37.084 | 51.765 | 52.885 | 1.00 | 153.43 |
| 1402 | NZ | LYS | 171 | 35.907 | 52.541 | 52.405 | 1.00 | 153.43 |
| 1403 | C | LYS | 171 | 39.042 | 47.737 | 56.189 | 1.00 | 166.26 |
| 1404 | O | LYS | 171 | 39.710 | 46.977 | 56.894 | 1.00 | 166.26 |
| 1405 | C1 | NAG | 221 | 52.176 | 13.407 | 48.424 | 1.00 | 124.69 |
| 1406 | C2 | NAG | 221 | 52.353 | 13.121 | 46.936 | 1.00 | 124.69 |
| 1407 | N2 | NAG | 221 | 51.119 | 13.440 | 46.226 | 1.00 | 124.69 |
| 1408 | C7 | NAG | 221 | 51.096 | 14.392 | 45.292 | 1.00 | 124.69 |
| 1409 | O7 | NAG | 221 | 52.111 | 14.981 | 44.911 | 1.00 | 124.69 |
| 1410 | C8 | NAG | 221 | 49.744 | 14.746 | 44.682 | 1.00 | 124.69 |
| 1411 | C3 | NAG | 221 | 52.712 | 11.631 | 46.753 | 1.00 | 124.69 |
| 1412 | O3 | NAG | 221 | 53.109 | 11.400 | 45.409 | 1.00 | 124.69 |
| 1413 | C4 | NAG | 221 | 53.847 | 11.168 | 47.703 | 1.00 | 124.69 |
| 1414 | O4 | NAG | 221 | 53.876 | 9.724 | 47.741 | 1.00 | 124.69 |
| 1415 | C5 | NAG | 221 | 53.635 | 11.683 | 49.139 | 1.00 | 124.69 |
| 1416 | O5 | NAG | 221 | 53.371 | 13.099 | 49.133 | 1.00 | 124.69 |
| 1417 | C6 | NAG | 221 | 54.853 | 11.458 | 50.023 | 1.00 | 124.69 |
| 1418 | O6 | NAG | 221 | 54.616 | 11.876 | 51.361 | 1.00 | 124.69 |
| 1419 | C1 | NAG | 222 | 55.008 | 9.074 | 47.260 | 1.00 | 186.41 |
| 1420 | C2 | NAG | 222 | 55.394 | 7.926 | 48.219 | 1.00 | 186.41 |
| 1421 | N2 | NAG | 222 | 55.812 | 8.470 | 49.500 | 1.00 | 186.41 |
| 1422 | C7 | NAG | 222 | 55.243 | 8.053 | 50.628 | 1.00 | 186.41 |
| 1423 | O7 | NAG | 222 | 54.288 | 7.270 | 50.654 | 1.00 | 186.41 |
| 1424 | C8 | NAG | 222 | 55.823 | 8.589 | 51.930 | 1.00 | 186.41 |
| 1425 | C3 | NAG | 222 | 56.531 | 7.079 | 47.623 | 1.00 | 186.41 |
| 1426 | O3 | NAG | 222 | 56.764 | 5.942 | 48.445 | 1.00 | 186.41 |
| 1427 | C4 | NAG | 222 | 56.174 | 6.622 | 46.205 | 1.00 | 186.41 |
| 1428 | O4 | NAG | 222 | 57.286 | 5.958 | 45.616 | 1.00 | 186.41 |
| 1429 | C5 | NAG | 222 | 55.775 | 7.830 | 45.345 | 1.00 | 186.41 |
| 1430 | O5 | NAG | 222 | 54.681 | 8.548 | 45.963 | 1.00 | 186.41 |
| 1431 | C6 | NAG | 222 | 55.302 | 7.412 | 43.963 | 1.00 | 186.41 |
| 1432 | O6 | NAG | 222 | 54.550 | 8.444 | 43.340 | 1.00 | 186.41 |
| 1433 | C1 | NAG | 242 | 36.605 | 17.603 | 61.014 | 1.00 | 57.79 |
| 1434 | C2 | NAG | 242 | 36.383 | 16.211 | 60.400 | 1.00 | 57.79 |
| 1435 | N2 | NAG | 242 | 37.564 | 15.387 | 60.550 | 1.00 | 57.79 |
| 1436 | C7 | NAG | 242 | 37.706 | 14.678 | 61.665 | 1.00 | 57.79 |
| 1437 | O7 | NAG | 242 | 36.949 | 14.833 | 62.624 | 1.00 | 57.79 |
| 1438 | C8 | NAG | 242 | 38.838 | 13.642 | 61.747 | 1.00 | 57.79 |
| 1439 | C3 | NAG | 242 | 36.035 | 16.395 | 58.924 | 1.00 | 57.79 |
| 1440 | O3 | NAG | 242 | 35.897 | 15.142 | 58.256 | 1.00 | 57.79 |
| 1441 | C4 | NAG | 242 | 34.754 | 17.226 | 58.828 | 1.00 | 57.79 |
| 1442 | O4 | NAG | 242 | 34.498 | 17.491 | 57.446 | 1.00 | 57.79 |
| 1443 | C5 | NAG | 242 | 34.988 | 18.570 | 59.547 | 1.00 | 57.79 |
| 1444 | O5 | NAG | 242 | 35.393 | 18.365 | 60.921 | 1.00 | 57.79 |
| 1445 | C6 | NAG | 242 | 33.781 | 19.492 | 59.556 | 1.00 | 57.79 |
| 1446 | O6 | NAG | 242 | 34.170 | 20.863 | 59.403 | 1.00 | 57.79 |
| 1447 | C1 | NAG | 243 | 33.499 | 16.811 | 56.792 | 1.00 | 110.47 |
| 1448 | C2 | NAG | 243 | 33.279 | 17.515 | 55.463 | 1.00 | 110.47 |
| 1449 | N2 | NAG | 243 | 32.859 | 18.887 | 55.667 | 1.00 | 110.47 |
| 1450 | C7 | NAG | 243 | 33.584 | 19.876 | 55.149 | 1.00 | 110.47 |
| 1451 | O7 | NAG | 243 | 34.659 | 19.682 | 54.574 | 1.00 | 110.47 |
| 1452 | C8 | NAG | 243 | 33.036 | 21.294 | 55.277 | 1.00 | 110.47 |
| 1453 | C3 | NAG | 243 | 32.273 | 16.758 | 54.610 | 1.00 | 110.47 |
| 1454 | O3 | NAG | 243 | 32.168 | 17.391 | 53.345 | 1.00 | 110.47 |
| 1455 | C4 | NAG | 243 | 32.746 | 15.314 | 54.413 | 1.00 | 110.47 |
| 1456 | O4 | NAG | 243 | 31.718 | 14.574 | 53.705 | 1.00 | 110.47 |
| 1457 | C5 | NAG | 243 | 33.038 | 14.665 | 55.789 | 1.00 | 110.47 |
| 1458 | O5 | NAG | 243 | 33.967 | 15.474 | 56.555 | 1.00 | 110.47 |
| 1459 | C6 | NAG | 243 | 33.671 | 13.290 | 55.655 | 1.00 | 110.47 |
| 1460 | O6 | NAG | 243 | 34.005 | 12.749 | 56.924 | 1.00 | 110.47 |
| 1461 | C1 | MAN | 244 | 32.107 | 13.609 | 52.777 | 1.00 | 99.82 |
| 1462 | C2 | MAN | 244 | 31.311 | 12.313 | 53.039 | 1.00 | 99.82 |
| 1463 | O2 | MAN | 244 | 29.925 | 12.615 | 53.134 | 1.00 | 99.82 |
| 1464 | C3 | MAN | 244 | 31.545 | 11.278 | 51.921 | 1.00 | 99.82 |
| 1465 | O3 | MAN | 244 | 30.713 | 10.135 | 52.110 | 1.00 | 99.82 |
| 1466 | C4 | MAN | 244 | 31.266 | 11.903 | 50.555 | 1.00 | 99.82 |
| 1467 | O4 | MAN | 244 | 31.547 | 10.959 | 49.527 | 1.00 | 99.82 |
| 1468 | C5 | MAN | 244 | 32.168 | 13.132 | 50.412 | 1.00 | 99.82 |
| 1469 | O5 | MAN | 244 | 31.840 | 14.107 | 51.442 | 1.00 | 99.82 |
| 1470 | C6 | MAN | 244 | 32.132 | 13.816 | 49.038 | 1.00 | 99.82 |
| 1471 | O6 | MAN | 244 | 30.954 | 14.583 | 48.837 | 1.00 | 99.82 |
| 1472 | C1 | NAG | 250 | 57.134 | 13.804 | 64.271 | 1.00 | 196.94 |
| 1473 | C2 | NAG | 250 | 57.130 | 13.286 | 65.723 | 1.00 | 196.94 |
| 1474 | N2 | NAG | 250 | 58.492 | 13.078 | 66.175 | 1.00 | 196.94 |
| 1475 | C7 | NAG | 250 | 58.871 | 13.481 | 67.385 | 1.00 | 196.94 |
| 1476 | O7 | NAG | 250 | 58.184 | 14.217 | 68.096 | 1.00 | 196.94 |
| 1477 | C8 | NAG | 250 | 60.225 | 12.994 | 67.883 | 1.00 | 196.94 |
| 1478 | C3 | NAG | 250 | 56.359 | 11.960 | 65.803 | 1.00 | 196.94 |
| 1479 | O3 | NAG | 250 | 56.224 | 11.547 | 67.156 | 1.00 | 196.94 |
| 1480 | C4 | NAG | 250 | 54.974 | 12.098 | 65.176 | 1.00 | 196.94 |
| 1481 | O4 | NAG | 250 | 54.343 | 10.826 | 65.136 | 1.00 | 196.94 |
| 1482 | C5 | NAG | 250 | 55.102 | 12.658 | 63.758 | 1.00 | 196.94 |
| 1483 | O5 | NAG | 250 | 55.794 | 13.927 | 63.783 | 1.00 | 196.94 |
| 1484 | C6 | NAG | 250 | 53.754 | 12.893 | 63.115 | 1.00 | 196.94 |
| 1485 | O6 | NAG | 250 | 53.895 | 13.367 | 61.783 | 1.00 | 196.94 |
| 1486 | C1 | NAG | 274 | 45.966 | 34.168 | 75.904 | 1.00 | 202.51 |
| 1487 | C2 | NAG | 274 | 44.449 | 34.481 | 75.778 | 1.00 | 202.51 |
| 1488 | N2 | NAG | 274 | 44.020 | 34.633 | 74.386 | 1.00 | 202.51 |
| 1489 | C7 | NAG | 274 | 42.782 | 34.284 | 74.009 | 1.00 | 202.51 |
| 1490 | O7 | NAG | 274 | 42.000 | 33.685 | 74.755 | 1.00 | 202.51 |
| 1491 | C8 | NAG | 274 | 42.322 | 34.648 | 72.599 | 1.00 | 202.51 |
| 1492 | C3 | NAG | 274 | 44.167 | 35.773 | 76.602 | 1.00 | 202.51 |
| 1493 | O3 | NAG | 274 | 42.768 | 35.996 | 76.692 | 1.00 | 202.51 |
| 1494 | C4 | NAG | 274 | 44.757 | 35.723 | 78.040 | 1.00 | 202.51 |
| 1495 | O4 | NAG | 274 | 44.775 | 37.037 | 78.589 | 1.00 | 202.51 |
| 1496 | C5 | NAG | 274 | 46.191 | 35.157 | 78.058 | 1.00 | 202.51 |
| 1497 | O5 | NAG | 274 | 46.265 | 33.950 | 77.282 | 1.00 | 202.51 |
| 1498 | C6 | NAG | 274 | 46.690 | 34.798 | 79.448 | 1.00 | 202.51 |
| 1499 | O6 | NAG | 274 | 47.729 | 33.828 | 79.381 | 1.00 | 202.51 |
| 1500 | C1 | NAG | 340 | 47.734 | 48.240 | 47.742 | 1.00 | 87.46 |
| 1501 | C2 | NAG | 340 | 49.212 | 48.677 | 47.819 | 1.00 | 87.46 |
| 1502 | N2 | NAG | 340 | 50.123 | 47.546 | 47.707 | 1.00 | 87.46 |
| 1503 | C7 | NAG | 340 | 50.634 | 47.210 | 46.522 | 1.00 | 87.46 |
| 1504 | O7 | NAG | 340 | 50.025 | 47.375 | 45.468 | 1.00 | 87.46 |
| 1505 | C8 | NAG | 340 | 52.024 | 46.596 | 46.490 | 1.00 | 87.46 |
| 1506 | C3 | NAG | 340 | 49.416 | 49.457 | 49.129 | 1.00 | 87.46 |
| 1507 | O3 | NAG | 340 | 50.779 | 49.830 | 49.261 | 1.00 | 87.46 |
| 1508 | C4 | NAG | 340 | 48.512 | 50.694 | 49.007 | 1.00 | 87.46 |
| 1509 | O4 | NAG | 340 | 48.730 | 51.749 | 49.989 | 1.00 | 87.46 |
| 1510 | C5 | NAG | 340 | 47.044 | 50.277 | 48.965 | 1.00 | 87.46 |
| 1511 | O5 | NAG | 340 | 46.834 | 49.391 | 47.812 | 1.00 | 87.46 |
| 1512 | C6 | NAG | 340 | 46.182 | 51.556 | 48.793 | 1.00 | 87.46 |
| 1513 | O6 | NAG | 340 | 44.848 | 51.307 | 48.368 | 1.00 | 87.46 |
| 1514 | C1 | NAG | 341 | 49.306 | 51.566 | 51.238 | 1.00 | 143.93 |
| 1515 | C2 | NAG | 341 | 50.167 | 52.801 | 51.506 | 1.00 | 143.93 |
| 1516 | N2 | NAG | 341 | 51.241 | 52.873 | 50.540 | 1.00 | 143.93 |
| 1517 | C7 | NAG | 341 | 51.195 | 53.774 | 49.564 | 1.00 | 143.93 |
| 1518 | O7 | NAG | 341 | 50.313 | 54.630 | 49.471 | 1.00 | 143.93 |
| 1519 | C8 | NAG | 341 | 52.303 | 53.716 | 48.535 | 1.00 | 143.93 |
| 1520 | C3 | NAG | 341 | 50.722 | 52.777 | 52.923 | 1.00 | 143.93 |
| 1521 | O3 | NAG | 341 | 51.522 | 53.931 | 53.148 | 1.00 | 143.93 |
| 1522 | C4 | NAG | 341 | 49.548 | 52.753 | 53.891 | 1.00 | 143.93 |
| 1523 | O4 | NAG | 341 | 50.031 | 52.717 | 55.229 | 1.00 | 143.93 |
| 1524 | C5 | NAG | 341 | 48.686 | 51.510 | 53.587 | 1.00 | 143.93 |
| 1525 | O5 | NAG | 341 | 48.239 | 51.510 | 52.203 | 1.00 | 143.93 |
| 1526 | C6 | NAG | 341 | 47.428 | 51.437 | 54.427 | 1.00 | 143.93 |
| 1527 | O6 | NAG | 341 | 46.455 | 50.627 | 53.780 | 1.00 | 143.93 |
| 1528 | C1 | NAG | 366 | 28.633 | 34.916 | 48.881 | 1.00 | 149.17 |
| 1529 | C2 | NAG | 366 | 27.879 | 34.326 | 50.081 | 1.00 | 149.17 |
| 1530 | N2 | NAG | 366 | 28.118 | 32.897 | 50.186 | 1.00 | 149.17 |
| 1531 | C7 | NAG | 366 | 28.345 | 32.346 | 51.378 | 1.00 | 149.17 |
| 1532 | O7 | NAG | 366 | 28.482 | 33.013 | 52.407 | 1.00 | 149.17 |
| 1533 | C8 | NAG | 366 | 28.441 | 30.828 | 51.448 | 1.00 | 149.17 |
| 1534 | C3 | NAG | 366 | 26.372 | 34.552 | 49.949 | 1.00 | 149.17 |
| 1535 | O3 | NAG | 366 | 25.761 | 34.256 | 51.198 | 1.00 | 149.17 |
| 1536 | C4 | NAG | 366 | 25.976 | 35.987 | 49.514 | 1.00 | 149.17 |
| 1537 | O4 | NAG | 366 | 24.660 | 35.921 | 48.921 | 1.00 | 149.17 |
| 1538 | C5 | NAG | 366 | 26.928 | 36.592 | 48.459 | 1.00 | 149.17 |
| 1539 | O5 | NAG | 366 | 28.320 | 36.323 | 48.766 | 1.00 | 149.17 |
| 1540 | C6 | NAG | 366 | 26.769 | 38.111 | 48.368 | 1.00 | 149.17 |
| 1541 | O6 | NAG | 366 | 27.829 | 38.807 | 49.016 | 1.00 | 149.17 |
| 1542 | C1 | NAG | 367 | 23.729 | 36.910 | 49.196 | 1.00 | 173.80 |
| 1543 | C2 | NAG | 367 | 22.797 | 37.075 | 47.975 | 1.00 | 173.80 |
| 1544 | N2 | NAG | 367 | 23.536 | 37.637 | 46.860 | 1.00 | 173.80 |
| 1545 | C7 | NAG | 367 | 23.834 | 36.896 | 45.796 | 1.00 | 173.80 |
| 1546 | O7 | NAG | 367 | 23.531 | 35.706 | 45.691 | 1.00 | 173.80 |
| 1547 | C8 | NAG | 367 | 24.586 | 37.593 | 44.674 | 1.00 | 173.80 |
| 1548 | C3 | NAG | 367 | 21.617 | 37.991 | 48.311 | 1.00 | 173.80 |
| 1549 | O3 | NAG | 367 | 20.711 | 38.025 | 47.218 | 1.00 | 173.80 |
| 1550 | C4 | NAG | 367 | 20.897 | 37.499 | 49.566 | 1.00 | 173.80 |
| 1551 | O4 | NAG | 367 | 19.890 | 38.438 | 49.924 | 1.00 | 173.80 |
| 1552 | C5 | NAG | 367 | 21.917 | 37.360 | 50.705 | 1.00 | 173.80 |
| 1553 | O5 | NAG | 367 | 22.977 | 36.460 | 50.326 | 1.00 | 173.80 |
| 1554 | C6 | NAG | 367 | 21.347 | 36.810 | 51.995 | 1.00 | 173.80 |
| 1555 | O6 | NAG | 367 | 22.385 | 36.606 | 52.948 | 1.00 | 173.80 |

TABLE 9
PhFcεRIα₁₋₁₇₂, Form T1, residue exposure
>>>> coordinate set = pent63_8c1.pdb

| segid | resid | resname | access | access-main | access-side |
|---|---|---|---|---|---|
| CCCC | 4 | LYS | 22.3151 | 10.9559 | 31.4026 |
| CCCC | 5 | PRO | 1.1153 | 1.4307 | 0.6949 |
| CCCC | 6 | LYS | 16.7221 | 1.1596 | 29.1721 |
| CCCC | 7 | VAL | 1.5573 | 2.7252 | 0.0000 |
| CCCC | 8 | SER | 8.9731 | 1.8795 | 23.1603 |
| CCCC | 9 | LEU | 3.7370 | 4.7824 | 2.6917 |
| CCCC | 10 | ASN | 12.6673 | 0.9406 | 24.3940 |
| CCCC | 11 | PRO | 8.2815 | 0.5829 | 18.5464 |
| CCCC | 12 | PRO | 9.7742 | 2.0935 | 20.0152 |
| CCCC | 13 | TRP | 1.5926 | 0.1230 | 2.1805 |
| CCCC | 14 | ASN | 3.3766 | 0.2934 | 6.4597 |
| CCCC | 15 | ARG | 1.6352 | 0.0000 | 2.5696 |
| CCCC | 16 | ILE | 1.1737 | 0.0003 | 2.3470 |
| CCCC | 17 | PHE | 0.2696 | 0.0000 | 0.4237 |
| CCCC | 18 | LYS | 8.1283 | 3.2126 | 12.0608 |
| CCCC | 19 | GLY | 5.5800 | 5.5800 | 0.0000 |
| CCCC | 20 | GLU | 3.3428 | 0.0000 | 6.0170 |
| CCCC | 21 | ASN | 5.3342 | 3.9503 | 6.7182 |
| CCCC | 22 | VAL | 0.3267 | 0.4564 | 0.1538 |
| CCCC | 23 | THR | 5.3278 | 0.0157 | 12.4107 |
| CCCC | 24 | LEU | 0.2562 | 0.0002 | 0.5121 |
| CCCC | 25 | THR | 4.7853 | 0.0000 | 11.1657 |
| CCCC | 26 | CYS | 0.2343 | 0.3249 | 0.0530 |
| CCCC | 27 | ASN | 7.7637 | 1.8546 | 13.6728 |
| CCCC | 28 | GLY | 7.9103 | 7.9103 | 0.0000 |
| CCCC | 29 | ASN | 16.6538 | 7.7758 | 25.5318 |
| CCCC | 30 | ASN | 14.2106 | 9.9392 | 18.4821 |
| CCCC | 31 | PHE | 18.4293 | 8.6833 | 23.9984 |
| CCCC | 32 | PHE | 6.9543 | 6.9847 | 6.9370 |
| CCCC | 33 | GLU | 17.3275 | 4.6057 | 27.5049 |
| CCCC | 34 | VAL | 9.7070 | 3.0781 | 18.5455 |
| CCCC | 35 | SER | 14.3512 | 2.1631 | 38.7274 |
| CCCC | 36 | SER | 7.0113 | 1.9003 | 17.2334 |
| CCCC | 37 | THR | 0.7139 | 1.2493 | 0.0000 |
| CCCC | 38 | LYS | 8.3149 | 0.3194 | 14.7113 |
| CCCC | 39 | TRP | 0.0064 | 0.0013 | 0.0084 |
| CCCC | 40 | PHE | 3.0089 | 0.0000 | 4.7283 |
| CCCC | 41 | HIS | 3.3635 | 0.3462 | 5.3750 |
| CCCC | 42 | ASN | 5.9924 | 6.1741 | 5.8107 |
| CCCC | 43 | GLY | 8.7956 | 8.7956 | 0.0000 |
| CCCC | 44 | SER | 10.0868 | 2.4356 | 25.3890 |
| CCCC | 45 | LEU | 14.4496 | 6.7421 | 22.1571 |
| CCCC | 46 | SER | 4.8664 | 3.2655 | 8.0682 |
| CCCC | 47 | GLU | 13.8158 | 4.4486 | 21.3095 |
| CCCC | 48 | GLU | 3.7957 | 0.7742 | 6.2129 |
| CCCC | 49 | THR | 11.0308 | 0.0826 | 25.6285 |
| CCCC | 50 | ASN | 3.7680 | 0.9608 | 6.5753 |
| CCCC | 51 | SER | 0.9943 | 0.0001 | 2.9826 |
| CCCC | 52 | SER | 2.8849 | 0.0003 | 8.6541 |
| CCCC | 53 | LEU | 2.6956 | 0.0208 | 5.3704 |
| CCCC | 54 | ASN | 7.0487 | 3.7820 | 10.3153 |
| CCCC | 55 | ILE | 2.0484 | 1.5241 | 2.5727 |
| CCCC | 56 | VAL | 8.3718 | 1.8780 | 17.0302 |
| CCCC | 57 | ASN | 6.8809 | 0.9872 | 12.7747 |
| CCCC | 58 | ALA | 0.3689 | 0.4454 | 0.0628 |
| CCCC | 59 | LYS | 8.8541 | 0.0311 | 15.9126 |
| CCCC | 60 | PHE | 3.1986 | 0.2109 | 4.9058 |
| CCCC | 61 | GLU | 8.5928 | 0.1580 | 15.3407 |
| CCCC | 62 | ASP | 4.2001 | 0.0802 | 8.3200 |
| CCCC | 63 | SER | 0.1586 | 0.0000 | 0.4759 |
| CCCC | 64 | GLY | 0.0205 | 0.0205 | 0.0000 |
| CCCC | 65 | GLU | 3.1362 | 0.1046 | 5.5614 |
| CCCC | 66 | TYR | 0.7765 | 0.0000 | 1.1648 |
| CCCC | 67 | LYS | 3.3029 | 0.0005 | 5.9449 |
| CCCC | 68 | CYS | 0.0000 | 0.0000 | 0.0000 |
| CCCC | 69 | GLN | 3.8782 | 0.0000 | 6.9808 |
| CCCC | 70 | HIS | 2.2973 | 0.2891 | 3.6361 |
| CCCC | 71 | GLN | 15.5567 | 6.4047 | 22.8783 |
| CCCC | 72 | GLN | 18.9536 | 6.5187 | 28.9015 |
| CCCC | 73 | VAL | 6.3773 | 2.6597 | 11.3340 |
| CCCC | 74 | ASN | 7.2490 | 0.8511 | 13.6469 |
| CCCC | 75 | GLU | 9.5776 | 4.4490 | 13.6806 |
| CCCC | 76 | SER | 0.7222 | 1.0831 | 0.0006 |
| CCCC | 77 | GLU | 13.3681 | 1.0686 | 23.2077 |
| CCCC | 78 | PRO | 2.7891 | 1.9776 | 3.8710 |
| CCCC | 79 | VAL | 5.1775 | 0.7766 | 11.0455 |
| CCCC | 80 | TYR | 3.7512 | 1.4544 | 4.8996 |
| CCCC | 81 | LEU | 0.2610 | 0.0000 | 0.5221 |
| CCCC | 82 | GLU | 5.7107 | 0.0011 | 10.2784 |
| CCCC | 83 | VAL | 1.5280 | 2.6739 | 0.0000 |
| CCCC | 84 | PHE | 2.6808 | 0.7184 | 3.8022 |
| CCCC | 85 | SER | 5.8787 | 5.8008 | 6.0345 |
| CCCC | 86 | ASP | 6.0903 | 2.6973 | 9.4832 |
| CCCC | 87 | TRP | 3.0930 | 0.2603 | 4.2261 |
| CCCC | 88 | LEU | 0.0403 | 0.0675 | 0.0131 |
| CCCC | 89 | LEU | 0.1021 | 0.0000 | 0.2041 |
| CCCC | 90 | LEU | 0.0000 | 0.0000 | 0.0000 |
| CCCC | 91 | GLN | 0.2597 | 0.0000 | 0.4674 |
| CCCC | 92 | ALA | 0.0596 | 0.0563 | 0.0730 |
| CCCC | 93 | SER | 4.6788 | 3.0077 | 8.0211 |
| CCCC | 94 | ALA | 8.5911 | 1.3052 | 37.7350 |
| CCCC | 95 | GLU | 4.4767 | 1.5831 | 6.7916 |
| CCCC | 96 | VAL | 4.3906 | 4.6557 | 4.0371 |
| CCCC | 97 | VAL | 1.4394 | 1.1406 | 1.8378 |
| CCCC | 98 | MET | 13.6889 | 0.2044 | 27.1734 |
| CCCC | 99 | GLU | 7.4797 | 4.8677 | 9.5692 |
| CCCC | 100 | GLY | 5.3567 | 5.3567 | 0.0000 |
| CCCC | 101 | GLN | 9.7722 | 0.0006 | 17.5894 |
| CCCC | 102 | PRO | 9.4569 | 1.5764 | 19.9642 |
| CCCC | 103 | LEU | 0.0179 | 0.0357 | 0.0000 |
| CCCC | 104 | PHE | 6.2358 | 0.0000 | 9.7991 |
| CCCC | 105 | LEU | 0.0474 | 0.0488 | 0.0461 |
| CCCC | 106 | ARG | 2.3314 | 0.0000 | 3.6636 |
| CCCC | 107 | CYS | 0.7781 | 1.1549 | 0.0246 |
| CCCC | 108 | HIS | 1.2171 | 0.3012 | 1.8277 |
| CCCC | 109 | GLY | 1.2651 | 1.2651 | 0.0000 |
| CCCC | 110 | TRP | 1.9508 | 0.3094 | 2.6074 |
| CCCC | 111 | ARG | 7.1821 | 6.7612 | 7.4226 |
| CCCC | 112 | ASN | 12.7243 | 3.8235 | 21.6251 |
| CCCC | 113 | TRP | 2.9331 | 3.2961 | 2.7878 |
| CCCC | 114 | ASP | 11.7314 | 2.0501 | 21.4128 |
| CCCC | 115 | VAL | 0.8918 | 0.6165 | 1.2589 |
| CCCC | 116 | TYR | 5.7191 | 0.0000 | 8.5787 |
| CCCC | 117 | LYS | 10.9908 | 0.9471 | 19.0258 |
| CCCC | 118 | VAL | 0.0001 | 0.0000 | 0.0002 |
| CCCC | 119 | ILE | 4.7127 | 0.0007 | 9.4248 |
| CCCC | 120 | TYR | 0.0060 | 0.0000 | 0.0091 |
| CCCC | 121 | TYR | 3.6424 | 0.0150 | 5.4562 |
| CCCC | 122 | LYS | 3.9385 | 0.8428 | 6.4150 |
| CCCC | 123 | ASP | 11.0597 | 7.2355 | 14.8840 |
| CCCC | 124 | GLY | 13.5829 | 13.5829 | 0.0000 |
| CCCC | 125 | GLU | 13.1544 | 0.5211 | 23.2611 |
| CCCC | 126 | ALA | 15.0490 | 5.4493 | 53.4477 |
| CCCC | 127 | LEU | 9.4150 | 6.1124 | 12.7176 |
| CCCC | 128 | LYS | 11.5717 | 1.7494 | 19.4295 |
| CCCC | 129 | TYR | 10.5011 | 5.5905 | 12.9565 |
| CCCC | 130 | TRP | 8.0873 | 0.9625 | 10.9373 |
| CCCC | 131 | TYR | 11.7870 | 1.0734 | 17.1438 |
| CCCC | 132 | GLU | 12.6705 | 2.2279 | 21.0247 |
| CCCC | 133 | ASN | 5.3027 | 5.3599 | 5.2454 |
| CCCC | 134 | HIS | 8.2476 | 1.2608 | 12.9055 |
| CCCC | 135 | ASN | 1.2965 | 0.3213 | 2.2717 |
| CCCC | 136 | ILE | 2.0165 | 1.3778 | 2.6552 |
| CCCC | 137 | SER | 9.9968 | 7.2656 | 15.4593 |
| CCCC | 138 | ILE | 3.6077 | 0.9873 | 6.2280 |
| CCCC | 139 | THR | 15.8360 | 2.4317 | 33.7085 |
| CCCC | 140 | ASN | 6.0823 | 3.6720 | 8.4926 |
| CCCC | 141 | ALA | 0.0000 | 0.0000 | 0.0000 |
| CCCC | 142 | THR | 6.7820 | 0.1381 | 15.6405 |
| CCCC | 143 | VAL | 5.0630 | 1.4175 | 9.9237 |
| CCCC | 144 | GLU | 14.1160 | 4.3532 | 21.9263 |
| CCCC | 145 | ASP | 4.3317 | 0.0259 | 8.6374 |
| CCCC | 146 | SER | 5.1283 | 3.0010 | 9.3829 |
| CCCC | 147 | GLY | 3.4210 | 3.4210 | 0.0000 |
| CCCC | 148 | THR | 5.2803 | 0.0914 | 12.1988 |
| CCCC | 149 | TYR | 0.2014 | 0.0000 | 0.3021 |
| CCCC | 150 | TYR | 3.7574 | 0.0000 | 5.6362 |
| CCCC | 151 | CYS | 0.0001 | 0.0001 | 0.0000 |
| CCCC | 152 | THR | 3.8919 | 0.0107 | 9.0668 |
| CCCC | 153 | GLY | 1.0188 | 1.0188 | 0.0000 |
| CCCC | 154 | LYS | 6.4238 | 0.0528 | 11.5207 |
| CCCC | 155 | VAL | 0.4180 | 0.0000 | 0.9754 |
| CCCC | 156 | TRP | 3.3279 | 3.7718 | 3.1504 |
| CCCC | 157 | GLN | 13.1268 | 3.2479 | 21.0299 |
| CCCC | 158 | LEU | 8.7018 | 0.1257 | 17.2778 |
| CCCC | 159 | ASP | 14.2676 | 4.9595 | 23.5758 |
| CCCC | 160 | TYR | 2.2687 | 2.5573 | 2.1243 |
| CCCC | 161 | GLU | 12.1767 | 4.4230 | 18.3798 |
| CCCC | 162 | SER | 1.1841 | 1.7762 | 0.0000 |
| CCCC | 163 | GLU | 9.4913 | 0.1747 | 16.9445 |
| CCCC | 164 | PRO | 9.7765 | 1.5124 | 20.7953 |
| CCCC | 165 | LEU | 1.6495 | 0.1289 | 3.1701 |
| CCCC | 166 | ASN | 3.4007 | 0.7824 | 6.0190 |
| CCCC | 167 | ILE | 0.5293 | 1.0585 | 0.0000 |
| CCCC | 168 | THR | 3.2321 | 0.0380 | 7.4909 |
| CCCC | 169 | VAL | 0.0723 | 0.1266 | 0.0000 |
| CCCC | 170 | ILE | 2.2951 | 0.0689 | 4.5213 |
| CCCC | 171 | LYS | 14.3432 | 12.9256 | 15.4773 |
| CCCC | 221 | NAG | 11.7001 | 0.0000 | 11.7001 |
| CCCC | 222 | NAG | 14.4010 | 0.0000 | 14.4010 |
| CCCC | 242 | NAG | 7.1046 | 0.0000 | 7.1046 |
| CCCC | 243 | NAG | 8.0078 | 0.0000 | 8.0078 |
| CCCC | 244 | MAN | 16.5438 | 0.0000 | 16.5438 |
| CCCC | 250 | NAG | 16.2147 | 0.0000 | 16.2147 |
| CCCC | 274 | NAG | 21.7742 | 0.0000 | 21.7742 |
| CCCC | 335 | NAG | 15.0979 | 0.0000 | 15.0979 |
| CCCC | 340 | NAG | 17.6065 | 0.0000 | 17.6065 |
| CCCC | 366 | NAG | 11.8776 | 0.0000 | 11.8776 |
| CCCC | 367 | NAG | 19.0810 | 0.0000 | 19.0810 |
| AAAA | 4 | LYS | 15.9363 | 6.7194 | 23.3099 |
| AAAA | 5 | PRO | 1.1488 | 1.4481 | 0.7498 |
| AAAA | 6 | LYS | 13.8574 | 1.1896 | 23.9916 |
| AAAA | 7 | VAL | 1.5646 | 2.7375 | 0.0007 |
| AAAA | 8 | SER | 9.0558 | 1.9390 | 23.2893 |
| AAAA | 9 | LEU | 3.8393 | 4.9388 | 2.7398 |
| AAAA | 10 | ASN | 12.5152 | 0.9543 | 24.0762 |
| AAAA | 11 | PRO | 8.3710 | 0.5349 | 18.8192 |
| AAAA | 12 | PRO | 9.8889 | 2.0781 | 20.3033 |
| AAAA | 13 | TRP | 1.5673 | 0.1143 | 2.1485 |
| AAAA | 14 | ASN | 3.1275 | 0.2808 | 5.9741 |
| AAAA | 15 | ARG | 1.6130 | 0.0057 | 2.5315 |
| AAAA | 16 | ILE | 1.1993 | 0.0000 | 2.3986 |
| AAAA | 17 | PHE | 0.2501 | 0.0000 | 0.3929 |
| AAAA | 18 | LYS | 10.8021 | 3.4649 | 16.6720 |
| AAAA | 19 | GLY | 5.6939 | 5.6939 | 0.0000 |
| AAAA | 20 | GLU | 3.4960 | 0.0003 | 6.2926 |
| AAAA | 21 | ASN | 5.3970 | 4.0391 | 6.7548 |
| AAAA | 22 | VAL | 0.2660 | 0.3430 | 0.1633 |
| AAAA | 23 | THR | 5.2134 | 0.0418 | 12.1090 |
| AAAA | 24 | LEU | 0.2554 | 0.0007 | 0.5101 |
| AAAA | 25 | THR | 4.8655 | 0.0005 | 11.3521 |
| AAAA | 26 | CYS | 0.2307 | 0.3194 | 0.0532 |
| AAAA | 27 | ASN | 0.4259 | 0.2949 | 0.5569 |
| AAAA | 28 | GLY | 4.4162 | 4.4162 | 0.0000 |
| AAAA | 29 | ASN | 7.6150 | 7.1438 | 8.0862 |
| AAAA | 30 | ASN | 11.3939 | 10.0050 | 12.7829 |
| AAAA | 31 | PHE | 14.7556 | 4.8219 | 20.4320 |
| AAAA | 32 | PHE | 2.7147 | 4.5718 | 1.6536 |
| AAAA | 33 | GLU | 7.5248 | 4.4574 | 9.9787 |
| AAAA | 34 | VAL | 4.0858 | 3.0519 | 5.4642 |
| AAAA | 35 | SER | 14.4683 | 2.1629 | 39.0790 |
| AAAA | 36 | SER | 6.9471 | 1.9207 | 16.9999 |
| AAAA | 37 | THR | 0.7130 | 1.2478 | 0.0000 |
| AAAA | 38 | LYS | 8.2623 | 0.2915 | 14.6390 |
| AAAA | 39 | TRP | 0.0071 | 0.0080 | 0.0067 |
| AAAA | 40 | PHE | 2.9948 | 0.0014 | 4.7054 |
| AAAA | 41 | HIS | 3.3204 | 0.4762 | 5.2166 |
| AAAA | 42 | ASN | 6.4516 | 7.1677 | 5.7356 |
| AAAA | 43 | GLY | 9.6929 | 9.6929 | 0.0000 |
| AAAA | 44 | SER | 12.5467 | 3.0596 | 31.5209 |
| AAAA | 45 | LEU | 14.0597 | 6.3669 | 21.7524 |
| AAAA | 46 | SER | 5.9554 | 3.7890 | 10.2883 |
| AAAA | 47 | GLU | 19.8848 | 8.0231 | 29.3741 |
| AAAA | 48 | GLU | 6.1436 | 2.1551 | 9.3344 |
| AAAA | 49 | THR | 10.8974 | 0.0037 | 25.4223 |
| AAAA | 50 | ASN | 3.7283 | 0.9830 | 6.4736 |
| AAAA | 51 | SER | 1.0424 | 0.0007 | 3.1257 |
| AAAA | 52 | SER | 2.9027 | 0.0000 | 8.7080 |
| AAAA | 53 | LEU | 2.6315 | 0.0203 | 5.2426 |
| AAAA | 54 | ASN | 7.1111 | 3.9443 | 10.2778 |
| AAAA | 55 | ILE | 2.0993 | 1.5522 | 2.6464 |
| AAAA | 56 | VAL | 11.4229 | 1.8784 | 24.1489 |
| AAAA | 57 | ASN | 9.2772 | 0.9862 | 17.5682 |
| AAAA | 58 | ALA | 0.3197 | 0.3981 | 0.0062 |
| AAAA | 59 | LYS | 13.5164 | 0.0175 | 24.3155 |
| AAAA | 60 | PHE | 3.5563 | 0.1394 | 5.5088 |
| AAAA | 61 | GLU | 8.5716 | 0.1791 | 15.2857 |
| AAAA | 62 | ASP | 4.2001 | 0.0591 | 8.3412 |
| AAAA | 63 | SER | 0.1501 | 0.0000 | 0.4504 |
| AAAA | 64 | GLY | 0.0335 | 0.0335 | 0.0000 |
| AAAA | 65 | GLU | 8.1768 | 0.0971 | 14.6406 |
| AAAA | 66 | TYR | 0.7839 | 0.0002 | 1.1758 |
| AAAA | 67 | LYS | 3.2196 | 0.0004 | 5.7951 |
| AAAA | 68 | CYS | 0.0000 | 0.0000 | 0.0000 |
| AAAA | 69 | GLN | 3.8577 | 0.0000 | 6.9439 |
| AAAA | 70 | HIS | 0.2845 | 0.2473 | 0.3093 |
| AAAA | 71 | GLN | 14.8691 | 6.1965 | 21.8072 |
| AAAA | 72 | GLN | 18.3340 | 6.2079 | 28.0348 |
| AAAA | 73 | VAL | 3.2302 | 2.5357 | 4.1564 |
| AAAA | 74 | ASN | 7.3440 | 0.8492 | 13.8389 |
| AAAA | 75 | GLU | 9.5554 | 4.4097 | 13.6718 |
| AAAA | 76 | SER | 0.5321 | 0.7981 | 0.0000 |
| AAAA | 77 | GLU | 16.0334 | 0.9282 | 28.1176 |
| AAAA | 78 | PRO | 8.2987 | 5.2204 | 12.4031 |
| AAAA | 79 | VAL | 5.9326 | 1.3229 | 12.0789 |
| AAAA | 80 | TYR | 6.7460 | 1.9053 | 9.1664 |
| AAAA | 81 | LEU | 0.2734 | 0.0000 | 0.5469 |
| AAAA | 82 | GLU | 5.7587 | 0.0007 | 10.3651 |
| AAAA | 83 | VAL | 1.5339 | 2.6843 | 0.0000 |
| AAAA | 84 | PHE | 2.7544 | 0.6865 | 3.9361 |
| AAAA | 85 | SER | 11.1143 | 5.9578 | 21.4274 |
| AAAA | 86 | ASP | 6.1024 | 2.2574 | 9.9474 |
| AAAA | 87 | TRP | 9.2004 | 0.2983 | 12.7613 |
| AAAA | 88 | LEU | 0.0297 | 0.0543 | 0.0051 |
| AAAA | 89 | LEU | 0.0927 | 0.0000 | 0.1854 |
| AAAA | 90 | LEU | 0.0001 | 0.0000 | 0.0002 |
| AAAA | 91 | GLN | 0.2535 | 0.0000 | 0.4563 |
| AAAA | 92 | ALA | 0.0453 | 0.0480 | 0.0344 |
| AAAA | 93 | SER | 4.6084 | 2.9327 | 7.9597 |
| AAAA | 94 | ALA | 8.7098 | 1.2990 | 38.3529 |
| AAAA | 95 | GLU | 7.3017 | 1.5643 | 11.8916 |
| AAAA | 96 | VAL | 12.9692 | 4.7889 | 23.8762 |
| AAAA | 97 | VAL | 1.4036 | 1.0908 | 1.8207 |
| AAAA | 98 | MET | 7.3760 | 0.2425 | 14.5094 |
| AAAA | 99 | GLU | 7.1208 | 4.6443 | 9.1019 |
| AAAA | 100 | GLY | 5.2380 | 5.2380 | 0.0000 |
| AAAA | 101 | GLN | 3.2910 | 0.0195 | 5.9082 |
| AAAA | 102 | PRO | 5.1174 | 1.6052 | 9.8002 |
| AAAA | 103 | LEU | 0.0323 | 0.0600 | 0.0045 |
| AAAA | 104 | PHE | 6.3337 | 0.0009 | 9.9525 |
| AAAA | 105 | LEU | 0.0742 | 0.0597 | 0.0887 |
| AAAA | 106 | ARG | 2.3217 | 0.0000 | 3.6484 |
| AAAA | 107 | CYS | 0.7916 | 1.1873 | 0.0000 |
| AAAA | 108 | HIS | 1.2205 | 0.3014 | 1.8333 |
| AAAA | 109 | GLY | 1.3688 | 1.3688 | 0.0000 |
| AAAA | 110 | TRP | 4.3961 | 0.3129 | 6.0293 |
| AAAA | 111 | ARG | 14.1659 | 6.8259 | 18.3603 |
| AAAA | 112 | ASN | 12.3349 | 3.8047 | 20.8651 |
| AAAA | 113 | TRP | 7.3124 | 3.4188 | 8.8699 |
| AAAA | 114 | ASP | 11.5788 | 1.5704 | 21.5873 |
| AAAA | 115 | VAL | 0.8933 | 0.6941 | 1.1589 |
| AAAA | 116 | TYR | 5.1843 | 0.0006 | 7.7761 |
| AAAA | 117 | LYS | 7.1256 | 0.9679 | 12.0517 |
| AAAA | 118 | VAL | 0.0000 | 0.0000 | 0.0000 |
| AAAA | 119 | ILE | 1.4302 | 0.0000 | 2.8604 |
| AAAA | 120 | TYR | 0.0104 | 0.0005 | 0.0153 |
| AAAA | 121 | TYR | 2.8600 | 0.0167 | 4.2816 |
| AAAA | 122 | LYS | 3.8739 | 0.7828 | 6.3468 |
| AAAA | 123 | ASP | 11.0893 | 7.0588 | 15.1198 |
| AAAA | 124 | GLY | 13.7649 | 13.7649 | 0.0000 |
| AAAA | 125 | GLU | 8.1492 | 0.5254 | 14.2483 |
| AAAA | 126 | ALA | 1.3596 | 0.7744 | 3.7005 |
| AAAA | 127 | LEU | 5.0008 | 0.9822 | 9.0194 |
| AAAA | 128 | LYS | 10.8601 | 0.1501 | 19.4280 |
| AAAA | 129 | TYR | 5.7014 | 3.8333 | 6.6354 |
| AAAA | 130 | TRP | 7.7631 | 0.9621 | 10.4835 |
| AAAA | 131 | TYR | 3.0458 | 1.1141 | 4.0116 |
| AAAA | 132 | GLU | 11.1091 | 2.1808 | 18.2518 |
| AAAA | 133 | ASN | 5.2028 | 5.3196 | 5.0859 |
| AAAA | 134 | HIS | 8.3482 | 1.2156 | 13.1032 |
| AAAA | 135 | ASN | 1.2934 | 0.3190 | 2.2679 |
| AAAA | 136 | ILE | 2.1274 | 1.3695 | 2.8853 |
| AAAA | 137 | SER | 10.0348 | 7.2335 | 15.6375 |
| AAAA | 138 | ILE | 3.6211 | 1.0099 | 6.2322 |
| AAAA | 139 | THR | 16.0640 | 2.5806 | 34.0420 |
| AAAA | 140 | ASN | 5.4194 | 3.6273 | 7.2114 |
| AAAA | 141 | ALA | 0.0000 | 0.0000 | 0.0000 |
| AAAA | 142 | THR | 7.2278 | 0.4616 | 16.2495 |
| AAAA | 143 | VAL | 7.6811 | 1.4982 | 15.9250 |
| AAAA | 144 | GLU | 14.2939 | 4.1689 | 22.3939 |
| AAAA | 145 | ASP | 4.3509 | 0.0318 | 8.6701 |
| AAAA | 146 | SER | 5.2566 | 3.1044 | 9.5609 |
| AAAA | 147 | GLY | 3.2376 | 3.2376 | 0.0000 |
| AAAA | 148 | THR | 5.2658 | 0.0995 | 12.1542 |
| AAAA | 149 | TYR | 0.2165 | 0.0000 | 0.3248 |
| AAAA | 150 | TYR | 3.8830 | 0.0000 | 5.8245 |
| AAAA | 151 | CYS | 0.0000 | 0.0000 | 0.0000 |
| AAAA | 152 | THR | 3.7398 | 0.0010 | 8.7248 |
| AAAA | 153 | GLY | 1.0201 | 1.0201 | 0.0000 |
| AAAA | 154 | LYS | 4.0119 | 0.0425 | 7.1873 |
| AAAA | 155 | VAL | 0.4567 | 0.0000 | 1.0656 |
| AAAA | 156 | TRP | 11.1226 | 3.7204 | 14.0834 |
| AAAA | 157 | GLN | 8.2831 | 3.3029 | 12.2673 |
| AAAA | 158 | LEU | 13.8927 | 0.1434 | 27.6420 |
| AAAA | 159 | ASP | 9.7733 | 4.0346 | 15.5120 |
| AAAA | 160 | TYR | 3.4354 | 2.0054 | 4.1504 |
| AAAA | 161 | GLU | 8.2007 | 5.4400 | 10.4093 |
| AAAA | 162 | SER | 1.2173 | 1.8259 | 0.0002 |
| AAAA | 163 | GLU | 9.4751 | 0.3080 | 16.8088 |
| AAAA | 164 | PRO | 9.8187 | 1.5247 | 20.8774 |
| AAAA | 165 | LEU | 1.6583 | 0.0972 | 3.2194 |
| AAAA | 166 | ASN | 4.2239 | 1.2362 | 7.2117 |
| AAAA | 167 | ILE | 0.5672 | 1.1307 | 0.0038 |
| AAAA | 168 | THR | 9.3576 | 0.0604 | 21.7538 |
| AAAA | 169 | VAL | 0.5466 | 0.9510 | 0.0074 |
| AAAA | 170 | ILE | 10.6664 | 1.5666 | 19.7663 |
| AAAA | 171 | LYS | 20.0709 | 14.6286 | 24.4247 |
| AAAA | 221 | NAG | 13.0731 | 0.0000 | 13.0731 |
| AAAA | 222 | NAG | 19.9260 | 0.0000 | 19.9260 |
| AAAA | 242 | NAG | 10.0968 | 0.0000 | 10.0968 |
| AAAA | 243 | NAG | 9.7429 | 0.0000 | 9.7429 |
| AAAA | 244 | MAN | 16.5025 | 0.0000 | 16.5025 |
| AAAA | 250 | NAG | 16.0048 | 0.0000 | 16.0048 |
| AAAA | 274 | NAG | 21.9758 | 0.0000 | 21.9758 |
| AAAA | 335 | NAG | 15.0266 | 0.0000 | 15.0266 |
| AAAA | 340 | NAG | 10.2058 | 0.0000 | 10.2058 |
| AAAA | 366 | NAG | 14.2003 | 0.0000 | 14.2003 |
| AAAA | 367 | NAG | 21.1043 | 0.0000 | 21.1043 |
| BBBB | 4 | LYS | 21.2711 | 8.1950 | 31.7320 |
| BBBB | 5 | PRO | 0.9327 | 1.4134 | 0.2918 |
| BBBB | 6 | LYS | 13.5721 | 0.9858 | 23.6411 |
| BBBB | 7 | VAL | 1.5696 | 2.7468 | 0.0000 |
| BBBB | 8 | SER | 9.0540 | 1.8158 | 23.5305 |
| BBBB | 9 | LEU | 3.7548 | 4.6164 | 2.8932 |
| BBBB | 10 | ASN | 12.3838 | 0.9742 | 23.7933 |
| BBBB | 11 | PRO | 8.3839 | 0.5771 | 18.7929 |
| BBBB | 12 | PRO | 10.2255 | 2.2114 | 20.9110 |
| BBBB | 13 | TRP | 1.5767 | 0.1420 | 2.1505 |
| BBBB | 14 | ASN | 3.6856 | 0.2734 | 7.0977 |
| BBBB | 15 | ARG | 1.6517 | 0.0084 | 2.5908 |
| BBBB | 16 | ILE | 1.1539 | 0.0000 | 2.3079 |
| BBBB | 17 | PHE | 0.2627 | 0.0000 | 0.4128 |
| BBBB | 18 | LYS | 10.5872 | 3.1464 | 16.5399 |
| BBBB | 19 | GLY | 5.2452 | 5.2452 | 0.0000 |
| BBBB | 20 | GLU | 3.4004 | 0.0000 | 6.1208 |
| BBBB | 21 | ASN | 5.3165 | 3.8893 | 6.7437 |
| BBBB | 22 | VAL | 0.3290 | 0.4639 | 0.1492 |
| BBBB | 23 | THR | 5.3376 | 0.0419 | 12.3986 |
| BBBB | 24 | LEU | 0.2556 | 0.0000 | 0.5112 |
| BBBB | 25 | THR | 4.8687 | 0.0000 | 11.3603 |
| BBBB | 26 | CYS | 0.2112 | 0.2986 | 0.0364 |
| BBBB | 27 | ASN | 0.5141 | 0.1099 | 0.9184 |
| BBBB | 28 | GLY | 2.2181 | 2.2181 | 0.0000 |
| BBBB | 29 | ASN | 10.0991 | 5.9026 | 14.2956 |
| BBBB | 30 | ASN | 8.2629 | 8.5326 | 7.9932 |
| BBBB | 31 | PHE | 13.1098 | 3.1872 | 18.7798 |
| BBBB | 32 | PHE | 3.2118 | 5.7094 | 1.7846 |
| BBBB | 33 | GLU | 9.8599 | 4.6157 | 14.0553 |
| BBBB | 34 | VAL | 4.2409 | 3.1111 | 5.7472 |
| BBBB | 35 | SER | 14.4622 | 2.1340 | 39.1186 |
| BBBB | 36 | SER | 7.0903 | 1.9980 | 17.2748 |
| BBBB | 37 | THR | 0.7246 | 1.2681 | 0.0000 |
| BBBB | 38 | LYS | 8.3435 | 0.2936 | 14.7834 |
| BBBB | 39 | TRP | 0.0045 | 0.0000 | 0.0063 |
| BBBB | 40 | PHE | 3.0307 | 0.0013 | 4.7618 |
| BBBB | 41 | HIS | 3.2985 | 0.2952 | 5.3007 |
| BBBB | 42 | ASN | 3.9446 | 4.4215 | 3.4677 |
| BBBB | 43 | GLY | 6.4448 | 6.4448 | 0.0000 |
| BBBB | 44 | SER | 7.9300 | 1.5277 | 20.7347 |
| BBBB | 45 | LEU | 14.0360 | 6.3402 | 21.7318 |
| BBBB | 46 | SER | 5.3085 | 3.4386 | 9.0482 |
| BBBB | 47 | GLU | 19.8137 | 7.9994 | 29.2651 |
| BBBB | 48 | GLU | 6.1811 | 2.3816 | 9.2207 |
| BBBB | 49 | THR | 10.8045 | 0.0111 | 25.1957 |
| BBBB | 50 | ASN | 3.7967 | 1.0050 | 6.5885 |
| BBBB | 51 | SER | 1.0119 | 0.0000 | 3.0357 |
| BBBB | 52 | SER | 2.8985 | 0.0010 | 8.6934 |
| BBBB | 53 | LEU | 2.8664 | 0.0001 | 5.7327 |
| BBBB | 54 | ASN | 7.0968 | 3.9693 | 10.2244 |
| BBBB | 55 | ILE | 2.0503 | 1.4906 | 2.6099 |
| BBBB | 56 | VAL | 11.3539 | 1.8189 | 24.0671 |
| BBBB | 57 | ASN | 9.2620 | 1.0325 | 17.4916 |
| BBBB | 58 | ALA | 0.3961 | 0.4951 | 0.0000 |
| BBBB | 59 | LYS | 13.4689 | 0.0284 | 24.2213 |
| BBBB | 60 | PHE | 3.3978 | 0.2767 | 5.1813 |
| BBBB | 61 | GLU | 8.8117 | 0.1782 | 15.7185 |
| BBBB | 62 | ASP | 4.1350 | 0.0756 | 8.1943 |
| BBBB | 63 | SER | 0.1730 | 0.0000 | 0.5191 |
| BBBB | 64 | GLY | 0.0000 | 0.0000 | 0.0000 |
| BBBB | 65 | GLU | 6.5484 | 0.0001 | 11.7871 |
| BBBB | 66 | TYR | 0.7915 | 0.0000 | 1.1872 |
| BBBB | 67 | LYS | 3.2805 | 0.0000 | 5.9049 |
| BBBB | 68 | CYS | 0.0000 | 0.0000 | 0.0000 |
| BBBB | 69 | GLN | 3.8141 | 0.0000 | 6.8654 |
| BBBB | 70 | HIS | 0.1609 | 0.2608 | 0.0943 |
| BBBB | 71 | GLN | 10.6127 | 5.2959 | 14.8662 |
| BBBB | 72 | GLN | 8.4010 | 6.0152 | 10.3096 |
| BBBB | 73 | VAL | 2.9562 | 2.5768 | 3.4619 |
| BBBB | 74 | ASN | 7.3147 | 0.9982 | 13.6312 |
| BBBB | 75 | GLU | 9.4113 | 4.1701 | 13.6043 |
| BBBB | 76 | SER | 0.5684 | 0.8526 | 0.0000 |
| BBBB | 77 | GLU | 15.8600 | 1.0254 | 27.7278 |
| BBBB | 78 | PRO | 8.2998 | 5.0247 | 12.6666 |
| BBBB | 79 | VAL | 5.5759 | 1.3782 | 11.1729 |
| BBBB | 80 | TYR | 6.2317 | 1.9484 | 8.3734 |
| BBBB | 81 | LEU | 0.2490 | 0.0005 | 0.4975 |
| BBBB | 82 | GLU | 5.8300 | 0.0000 | 10.4940 |
| BBBB | 83 | VAL | 1.5904 | 2.7832 | 0.0000 |
| BBBB | 84 | PHE | 2.7220 | 0.7033 | 3.8756 |
| BBBB | 85 | SER | 10.7069 | 5.7965 | 20.5277 |
| BBBB | 86 | ASP | 5.9874 | 2.1888 | 9.7860 |
| BBBB | 87 | TRP | 9.1171 | 0.3262 | 12.6335 |
| BBBB | 88 | LEU | 0.0289 | 0.0578 | 0.0000 |
| BBBB | 89 | LEU | 0.0888 | 0.0000 | 0.1776 |
| BBBB | 90 | LEU | 0.0000 | 0.0000 | 0.0000 |
| BBBB | 91 | GLN | 0.2626 | 0.0000 | 0.4726 |
| BBBB | 92 | ALA | 0.0481 | 0.0399 | 0.0813 |
| BBBB | 93 | SER | 4.6672 | 2.9572 | 8.0872 |
| BBBB | 94 | ALA | 8.7256 | 1.3164 | 38.3623 |
| BBBB | 95 | GLU | 7.1764 | 1.5358 | 11.6889 |
| BBBB | 96 | VAL | 12.6418 | 4.7054 | 23.2237 |
| BBBB | 97 | VAL | 1.4061 | 1.1339 | 1.7691 |
| BBBB | 98 | MET | 11.9430 | 0.2070 | 23.6789 |
| BBBB | 99 | GLU | 7.2607 | 4.6466 | 9.3520 |
| BBBB | 100 | GLY | 5.1244 | 5.1244 | 0.0000 |
| BBBB | 101 | GLN | 4.0221 | 0.0000 | 7.2398 |
| BBBB | 102 | PRO | 7.3498 | 1.5529 | 15.0790 |
| BBBB | 103 | LEU | 0.0071 | 0.0138 | 0.0004 |
| BBBB | 104 | PHE | 6.2937 | 0.0007 | 9.8898 |
| BBBB | 105 | LEU | 0.0567 | 0.0603 | 0.0531 |
| BBBB | 106 | ARG | 2.3037 | 0.0000 | 3.6201 |
| BBBB | 107 | CYS | 0.8127 | 1.2098 | 0.0186 |
| BBBB | 108 | HIS | 1.1982 | 0.2627 | 1.8218 |
| BBBB | 109 | GLY | 1.2842 | 1.2842 | 0.0000 |
| BBBB | 110 | TRP | 4.4804 | 0.3255 | 6.1423 |
| BBBB | 111 | ARG | 14.4479 | 6.9241 | 18.7472 |
| BBBB | 112 | ASN | 12.2860 | 3.9115 | 20.6605 |
| BBBB | 113 | TRP | 7.0297 | 3.4023 | 8.4806 |
| BBBB | 114 | ASP | 11.5554 | 1.8648 | 21.2461 |
| BBBB | 115 | VAL | 0.8700 | 0.6238 | 1.1983 |
| BBBB | 116 | TYR | 5.8899 | 0.0000 | 8.8349 |
| BBBB | 117 | LYS | 11.0072 | 0.9540 | 19.0498 |
| BBBB | 118 | VAL | 0.0000 | 0.0000 | 0.0000 |
| BBBB | 119 | ILE | 4.7824 | 0.0000 | 9.5649 |
| BBBB | 120 | TYR | 0.0292 | 0.0003 | 0.0436 |
| BBBB | 121 | TYR | 3.6258 | 0.0127 | 5.4323 |
| BBBB | 122 | LYS | 3.8173 | 0.8234 | 6.2125 |
| BBBB | 123 | ASP | 11.1350 | 7.1239 | 15.1460 |
| BBBB | 124 | GLY | 13.9353 | 13.9353 | 0.0000 |
| BBBB | 125 | GLU | 13.4298 | 0.5494 | 23.7341 |
| BBBB | 126 | ALA | 15.0233 | 5.4413 | 53.3510 |
| BBBB | 127 | LEU | 9.2699 | 5.9635 | 12.5762 |
| BBBB | 128 | LYS | 11.6658 | 1.8096 | 19.5508 |
| BBBB | 129 | TYR | 10.3608 | 5.2815 | 12.9005 |
| BBBB | 130 | TRP | 8.0170 | 1.0064 | 10.8213 |
| BBBB | 131 | TYR | 11.7311 | 1.0656 | 17.0638 |
| BBBB | 132 | GLU | 12.8760 | 2.2402 | 21.3846 |
| BBBB | 133 | ASN | 5.2983 | 5.3131 | 5.2835 |
| BBBB | 134 | HIS | 7.9203 | 1.2177 | 12.3886 |
| BBBB | 135 | ASN | 1.3098 | 0.3200 | 2.2995 |
| BBBB | 136 | ILE | 2.0145 | 1.4037 | 2.6254 |
| BBBB | 137 | SER | 9.9370 | 7.1329 | 15.5453 |
| BBBB | 138 | ILE | 3.5989 | 0.9814 | 6.2165 |
| BBBB | 139 | THR | 16.3957 | 2.4548 | 34.9836 |
| BBBB | 140 | ASN | 6.1076 | 3.6820 | 8.5333 |
| BBBB | 141 | ALA | 0.0000 | 0.0000 | 0.0000 |
| BBBB | 142 | THR | 6.9409 | 0.3571 | 15.7193 |
| BBBB | 143 | VAL | 8.0179 | 1.4651 | 16.7550 |
| BBBB | 144 | GLU | 13.9749 | 4.2931 | 21.7203 |
| BBBB | 145 | ASP | 4.2546 | 0.0236 | 8.4857 |
| BBBB | 146 | SER | 5.2200 | 3.1447 | 9.3705 |
| BBBB | 147 | GLY | 3.2638 | 3.2638 | 0.0000 |
| BBBB | 148 | THR | 5.2033 | 0.0855 | 12.0270 |
| BBBB | 149 | TYR | 0.2349 | 0.0000 | 0.3523 |
| BBBB | 150 | TYR | 3.8297 | 0.0006 | 5.7443 |
| BBBB | 151 | CYS | 0.0001 | 0.0002 | 0.0000 |
| BBBB | 152 | THR | 3.8729 | 0.0115 | 9.0216 |
| BBBB | 153 | GLY | 1.0080 | 1.0080 | 0.0000 |
| BBBB | 154 | LYS | 6.2899 | 0.0456 | 11.2854 |
| BBBB | 155 | VAL | 0.4338 | 0.0000 | 1.0121 |
| BBBB | 156 | TRP | 10.7802 | 3.5331 | 13.6790 |
| BBBB | 157 | GLN | 13.5388 | 3.3217 | 21.7125 |
| BBBB | 158 | LEU | 13.7581 | 0.1277 | 27.3885 |
| BBBB | 159 | ASP | 13.8236 | 4.9295 | 22.7177 |
| BBBB | 160 | TYR | 3.3842 | 2.5049 | 3.8239 |
| BBBB | 161 | GLU | 12.8172 | 5.3317 | 18.8056 |
| BBBB | 162 | SER | 1.1166 | 1.6744 | 0.0011 |
| BBBB | 163 | GLU | 9.2415 | 0.1789 | 16.4915 |
| BBBB | 164 | PRO | 9.7119 | 1.5204 | 20.6339 |
| BBBB | 165 | LEU | 1.6353 | 0.0985 | 3.1722 |
| BBBB | 166 | ASN | 4.1241 | 1.1171 | 7.1311 |
| BBBB | 167 | ILE | 0.5678 | 1.1351 | 0.0006 |
| BBBB | 168 | THR | 9.4957 | 0.0238 | 22.1247 |
| BBBB | 169 | VAL | 0.5214 | 0.9124 | 0.0000 |
| BBBB | 170 | ILE | 10.7674 | 1.5460 | 19.9889 |
| BBBB | 171 | LYS | 19.3575 | 14.1022 | 23.5617 |
| BBBB | 221 | NAG | 13.1653 | 0.0000 | 13.1653 |
| BBBB | 222 | NAG | 20.0638 | 0.0000 | 20.0638 |
| BBBB | 242 | NAG | 5.8770 | 0.0000 | 5.8770 |
| BBBB | 243 | NAG | 6.5918 | 0.0000 | 6.5918 |
| BBBB | 244 | MAN | 16.4886 | 0.0000 | 16.4886 |
| BBBB | 250 | NAG | 16.1285 | 0.0000 | 16.1285 |
| BBBB | 274 | NAG | 20.2170 | 0.0000 | 20.2170 |
| BBBB | 335 | NAG | 14.9859 | 0.0000 | 14.9859 |
| BBBB | 340 | NAG | 17.5037 | 0.0000 | 17.5037 |
| BBBB | 366 | NAG | 14.5337 | 0.0000 | 14.5337 |
| BBBB | 367 | NAG | 21.0686 | 0.0000 | 21.0686 |
| DDDD | 4 | LYS | 22.4057 | 10.8330 | 31.6640 |
| DDDD | 5 | PRO | 1.1546 | 1.4746 | 0.7280 |
| DDDD | 6 | LYS | 17.0122 | 1.1737 | 29.6830 |
| DDDD | 7 | VAL | 1.5813 | 2.7670 | 0.0004 |
| DDDD | 8 | SER | 9.0050 | 1.8942 | 23.2265 |
| DDDD | 9 | LEU | 3.6933 | 4.6049 | 2.7818 |
| DDDD | 10 | ASN | 12.3091 | 1.0020 | 23.6161 |
| DDDD | 11 | PRO | 8.3171 | 0.5504 | 18.6727 |
| DDDD | 12 | PRO | 9.9864 | 2.1032 | 20.4973 |
| DDDD | 13 | TRP | 1.5517 | 0.0931 | 2.1352 |
| DDDD | 14 | ASN | 3.3780 | 0.3110 | 6.4451 |
| DDDD | 15 | ARG | 1.6343 | 0.0116 | 2.5615 |
| DDDD | 16 | ILE | 1.1547 | 0.0005 | 2.3088 |
| DDDD | 17 | PHE | 0.2492 | 0.0000 | 0.3916 |
| DDDD | 18 | LYS | 4.6732 | 2.0544 | 6.7683 |
| DDDD | 19 | GLY | 3.1162 | 3.1162 | 0.0000 |
| DDDD | 20 | GLU | 3.5200 | 0.0000 | 6.3359 |
| DDDD | 21 | ASN | 5.4287 | 3.8849 | 6.9725 |
| DDDD | 22 | VAL | 0.3155 | 0.4412 | 0.1479 |
| DDDD | 23 | THR | 5.1394 | 0.0324 | 11.9489 |
| DDDD | 24 | LEU | 0.2366 | 0.0000 | 0.4733 |
| DDDD | 25 | THR | 4.8738 | 0.0000 | 11.3721 |
| DDDD | 26 | CYS | 0.2520 | 0.3402 | 0.0757 |
| DDDD | 27 | ASN | 7.6624 | 1.9816 | 13.3432 |
| DDDD | 28 | GLY | 7.7316 | 7.7316 | 0.0000 |
| DDDD | 29 | ASN | 17.3095 | 8.4320 | 26.1871 |
| DDDD | 30 | ASN | 14.6001 | 10.1471 | 19.0532 |
| DDDD | 31 | PHE | 18.5570 | 8.7657 | 24.1521 |
| DDDD | 32 | PHE | 7.0815 | 6.8006 | 7.2420 |
| DDDD | 33 | GLU | 17.2171 | 4.7180 | 27.2164 |
| DDDD | 34 | VAL | 10.0324 | 3.1007 | 19.2746 |
| DDDD | 35 | SER | 14.2871 | 2.1039 | 38.6536 |
| DDDD | 36 | SER | 6.9328 | 1.7803 | 17.2376 |
| DDDD | 37 | THR | 0.7026 | 1.2295 | 0.0000 |
| DDDD | 38 | LYS | 8.3451 | 0.3160 | 14.7683 |
| DDDD | 39 | TRP | 0.0041 | 0.0023 | 0.0049 |
| DDDD | 40 | PHE | 2.9900 | 0.0009 | 4.6980 |
| DDDD | 41 | HIS | 3.3768 | 0.3997 | 5.3616 |
| DDDD | 42 | ASN | 6.4948 | 7.0880 | 5.9016 |
| DDDD | 43 | GLY | 9.5817 | 9.5817 | 0.0000 |
| DDDD | 44 | SER | 12.6350 | 2.9429 | 32.0194 |
| DDDD | 45 | LEU | 14.2578 | 6.4935 | 22.0220 |
| DDDD | 46 | SER | 5.6757 | 3.8029 | 9.4212 |
| DDDD | 47 | GLU | 19.9087 | 7.8667 | 29.5423 |
| DDDD | 48 | GLU | 6.0890 | 2.3899 | 9.0482 |
| DDDD | 49 | THR | 10.8314 | 0.0116 | 25.2577 |
| DDDD | 50 | ASN | 3.8373 | 0.9946 | 6.6800 |
| DDDD | 51 | SER | 0.9841 | 0.0000 | 2.9524 |
| DDDD | 52 | SER | 2.8765 | 0.0001 | 8.6293 |
| DDDD | 53 | LEU | 2.8376 | 0.0187 | 5.6565 |
| DDDD | 54 | ASN | 7.0382 | 3.8726 | 10.2037 |
| DDDD | 55 | ILE | 2.0467 | 1.4442 | 2.6492 |
| DDDD | 56 | VAL | 11.5050 | 1.8629 | 24.3611 |
| DDDD | 57 | ASN | 8.8180 | 1.0298 | 16.6062 |
| DDDD | 58 | ALA | 0.2350 | 0.2934 | 0.0018 |
| DDDD | 59 | LYS | 13.6844 | 0.0264 | 24.6108 |
| DDDD | 60 | PHE | 2.6066 | 0.0006 | 4.0957 |
| DDDD | 61 | GLU | 8.6110 | 0.1659 | 15.3671 |
| DDDD | 62 | ASP | 4.2057 | 0.0884 | 8.3230 |
| DDDD | 63 | SER | 0.1340 | 0.0000 | 0.4019 |
| DDDD | 64 | GLY | 0.0349 | 0.0349 | 0.0000 |
| DDDD | 65 | GLU | 8.1888 | 0.0852 | 14.6716 |
| DDDD | 66 | TYR | 0.7677 | 0.0000 | 1.1516 |
| DDDD | 67 | LYS | 3.2893 | 0.0000 | 5.9208 |
| DDDD | 68 | CYS | 0.0005 | 0.0000 | 0.0014 |
| DDDD | 69 | GLN | 3.8578 | 0.0000 | 6.9441 |
| DDDD | 70 | HIS | 2.2626 | 0.2237 | 3.6218 |
| DDDD | 71 | GLN | 15.3304 | 6.1240 | 22.6955 |
| DDDD | 72 | GLN | 18.9257 | 6.3446 | 28.9906 |
| DDDD | 73 | VAL | 6.4935 | 2.6517 | 11.6159 |
| DDDD | 74 | ASN | 7.2861 | 0.8315 | 13.7407 |
| DDDD | 75 | GLU | 9.5469 | 4.3906 | 13.6720 |
| DDDD | 76 | SER | 0.4742 | 0.7107 | 0.0011 |
| DDDD | 77 | GLU | 16.0719 | 0.9787 | 28.1465 |
| DDDD | 78 | PRO | 8.1673 | 5.2859 | 12.0091 |
| DDDD | 79 | VAL | 5.7531 | 1.2596 | 11.7443 |
| DDDD | 80 | TYR | 6.8446 | 2.1690 | 9.1823 |
| DDDD | 81 | LEU | 0.2183 | 0.0000 | 0.4367 |
| DDDD | 82 | GLU | 5.8647 | 0.0374 | 10.5265 |
| DDDD | 83 | VAL | 1.5404 | 2.6954 | 0.0005 |
| DDDD | 84 | PHE | 2.7805 | 0.6706 | 3.9861 |
| DDDD | 85 | SER | 6.8439 | 5.9428 | 8.6459 |
| DDDD | 86 | ASP | 6.0109 | 2.1275 | 9.8943 |
| DDDD | 87 | TRP | 4.6976 | 0.2328 | 6.4835 |
| DDDD | 88 | LEU | 0.0296 | 0.0530 | 0.0062 |
| DDDD | 89 | LEU | 0.0803 | 0.0002 | 0.1605 |
| DDDD | 90 | LEU | 0.0000 | 0.0000 | 0.0000 |
| DDDD | 91 | GLN | 0.2460 | 0.0003 | 0.4426 |
| DDDD | 92 | ALA | 0.0626 | 0.0754 | 0.0113 |
| DDDD | 93 | SER | 4.5712 | 2.8665 | 7.9807 |
| DDDD | 94 | ALA | 8.7178 | 1.3188 | 38.3138 |
| DDDD | 95 | GLU | 7.2886 | 1.5976 | 11.8415 |
| DDDD | 96 | VAL | 12.8114 | 4.6767 | 23.6578 |
| DDDD | 97 | VAL | 1.4641 | 1.1764 | 1.8477 |
| DDDD | 98 | MET | 13.4393 | 0.1457 | 26.7329 |
| DDDD | 99 | GLU | 7.1147 | 4.7251 | 9.0264 |
| DDDD | 100 | GLY | 5.3684 | 5.3684 | 0.0000 |
| DDDD | 101 | GLN | 9.8859 | 0.0000 | 17.7946 |
| DDDD | 102 | PRO | 9.5952 | 1.6262 | 20.2206 |
| DDDD | 103 | LEU | 0.0075 | 0.0150 | 0.0000 |
| DDDD | 104 | PHE | 6.3221 | 0.0000 | 9.9347 |
| DDDD | 105 | LEU | 0.0690 | 0.0712 | 0.0667 |
| DDDD | 106 | ARG | 2.3233 | 0.0001 | 3.6509 |
| DDDD | 107 | CYS | 0.8061 | 1.2091 | 0.0000 |
| DDDD | 108 | HIS | 1.1851 | 0.2808 | 1.7880 |
| DDDD | 109 | GLY | 1.2333 | 1.2333 | 0.0000 |
| DDDD | 110 | TRP | 0.7404 | 0.3219 | 0.9078 |
| DDDD | 111 | ARG | 7.9699 | 6.6124 | 8.7456 |
| DDDD | 112 | ASN | 12.5024 | 3.9088 | 21.0960 |
| DDDD | 113 | TRP | 2.0923 | 3.4514 | 1.5487 |
| DDDD | 114 | ASP | 11.6891 | 1.9841 | 21.3940 |
| DDDD | 115 | VAL | 0.8907 | 0.6666 | 1.1894 |
| DDDD | 116 | TYR | 3.5406 | 0.0000 | 5.3109 |
| DDDD | 117 | LYS | 7.4915 | 0.9137 | 12.7537 |
| DDDD | 118 | VAL | 0.0000 | 0.0000 | 0.0000 |
| DDDD | 119 | ILE | 1.5354 | 0.0000 | 3.0708 |
| DDDD | 120 | TYR | 0.0227 | 0.0000 | 0.0341 |
| DDDD | 121 | TYR | 3.0000 | 0.0000 | 4.5000 |
| DDDD | 122 | LYS | 3.8497 | 0.8215 | 6.2723 |
| DDDD | 123 | ASP | 11.0185 | 7.2080 | 14.8291 |
| DDDD | 124 | GLY | 13.8186 | 13.8186 | 0.0000 |
| DDDD | 125 | GLU | 7.1969 | 0.5597 | 12.5066 |
| DDDD | 126 | ALA | 1.6231 | 0.9637 | 4.2606 |
| DDDD | 127 | LEU | 5.5580 | 1.1317 | 9.9843 |
| DDDD | 128 | LYS | 11.0326 | 0.1803 | 19.7145 |
| DDDD | 129 | TYR | 6.0662 | 4.5993 | 6.7997 |
| DDDD | 130 | TRP | 6.9751 | 0.9821 | 9.3722 |
| DDDD | 131 | TYR | 3.2844 | 1.1357 | 4.3587 |
| DDDD | 132 | GLU | 10.5294 | 2.1545 | 17.2294 |
| DDDD | 133 | ASN | 5.3145 | 5.3786 | 5.2503 |
| DDDD | 134 | HIS | 8.2621 | 1.1783 | 12.9846 |
| DDDD | 135 | ASN | 1.2924 | 0.3403 | 2.2445 |
| DDDD | 136 | ILE | 2.0846 | 1.3968 | 2.7725 |
| DDDD | 137 | SER | 10.0460 | 7.2958 | 15.5465 |
| DDDD | 138 | ILE | 3.5719 | 0.9619 | 6.1819 |
| DDDD | 139 | THR | 16.0437 | 2.5113 | 34.0868 |
| DDDD | 140 | ASN | 6.1043 | 3.6342 | 8.5744 |
| DDDD | 141 | ALA | 0.0006 | 0.0000 | 0.0028 |
| DDDD | 142 | THR | 7.0414 | 0.4613 | 15.8150 |
| DDDD | 143 | VAL | 7.7914 | 1.5021 | 16.1770 |
| DDDD | 144 | GLU | 13.9850 | 4.2384 | 21.7824 |
| DDDD | 145 | ASP | 4.1793 | 0.0265 | 8.3322 |
| DDDD | 146 | SER | 5.2665 | 3.1409 | 9.5178 |
| DDDD | 147 | GLY | 3.2916 | 3.2916 | 0.0000 |
| DDDD | 148 | THR | 5.1835 | 0.0792 | 11.9891 |
| DDDD | 149 | TYR | 0.2058 | 0.0000 | 0.3087 |
| DDDD | 150 | TYR | 3.8607 | 0.0000 | 5.7910 |
| DDDD | 151 | CYS | 0.0000 | 0.0000 | 0.0000 |
| DDDD | 152 | THR | 3.9195 | 0.0065 | 9.1368 |
| DDDD | 153 | GLY | 1.0864 | 1.0864 | 0.0000 |
| DDDD | 154 | LYS | 5.0786 | 0.0302 | 9.1174 |
| DDDD | 155 | VAL | 0.4195 | 0.0000 | 0.9789 |
| DDDD | 156 | TRP | 1.3921 | 2.1135 | 1.1036 |
| DDDD | 157 | GLN | 5.1050 | 3.3675 | 6.4950 |
| DDDD | 158 | LEU | 13.2526 | 0.1201 | 26.3851 |
| DDDD | 159 | ASP | 7.9559 | 2.3964 | 13.5153 |
| DDDD | 160 | TYR | 2.2863 | 2.2919 | 2.2835 |
| DDDD | 161 | GLU | 10.7234 | 4.9235 | 15.3634 |
| DDDD | 162 | SER | 1.2506 | 1.8759 | 0.0000 |
| DDDD | 163 | GLU | 9.3458 | 0.2040 | 16.6593 |
| DDDD | 164 | PRO | 9.8122 | 1.5651 | 20.8083 |
| DDDD | 165 | LEU | 1.6668 | 0.0989 | 3.2347 |
| DDDD | 166 | ASN | 4.0892 | 1.1470 | 7.0314 |
| DDDD | 167 | ILE | 0.5690 | 1.1374 | 0.0006 |
| DDDD | 168 | THR | 9.4203 | 0.0533 | 21.9098 |
| DDDD | 169 | VAL | 0.5492 | 0.9611 | 0.0000 |
| DDDD | 170 | ILE | 10.5373 | 1.6298 | 19.4449 |
| DDDD | 171 | LYS | 19.4334 | 14.2443 | 23.5846 |
| DDDD | 221 | NAG | 12.4351 | 0.0000 | 12.4351 |
| DDDD | 222 | NAG | 14.2041 | 0.0000 | 14.2041 |
| DDDD | 242 | NAG | 9.7024 | 0.0000 | 9.7024 |
| DDDD | 243 | NAG | 9.7925 | 0.0000 | 9.7925 |
| DDDD | 244 | MAN | 16.4248 | 0.0000 | 16.4248 |
| DDDD | 250 | NAG | 15.9655 | 0.0000 | 15.9655 |
| DDDD | 274 | NAG | 21.7485 | 0.0000 | 21.7485 |
| DDDD | 335 | NAG | 15.0635 | 0.0000 | 15.0635 |
| DDDD | 340 | NAG | 17.6569 | 0.0000 | 17.6569 |
| DDDD | 366 | NAG | 14.5792 | 0.0000 | 14.5792 |
| DDDD | 367 | NAG | 20.8687 | 0.0000 | 20.8687 |
| EEEE | 4 | LYS | 22.3558 | 10.9740 | 31.4612 |
| EEEE | 5 | PRO | 1.1163 | 1.4301 | 0.6978 |
| EEEE | 6 | LYS | 16.9326 | 1.2182 | 29.5041 |
| EEEE | 7 | VAL | 1.5370 | 2.6897 | 0.0000 |
| EEEE | 8 | SER | 9.0470 | 1.8198 | 23.5014 |
| EEEE | 9 | LEU | 3.7362 | 4.7200 | 2.7524 |
| EEEE | 10 | ASN | 12.0492 | 0.9478 | 23.1505 |
| EEEE | 11 | PRO | 8.3799 | 0.5532 | 18.8156 |
| EEEE | 12 | PRO | 9.8266 | 2.0198 | 20.2356 |
| EEEE | 13 | TRP | 1.5826 | 0.0955 | 2.1775 |
| EEEE | 14 | ASN | 3.6101 | 0.2979 | 6.9223 |
| EEEE | 15 | ARG | 1.6218 | 0.0098 | 2.5429 |
| EEEE | 16 | ILE | 1.1456 | 0.0000 | 2.2912 |
| EEEE | 17 | PHE | 0.2563 | 0.0000 | 0.4027 |
| EEEE | 18 | LYS | 10.9294 | 3.6992 | 16.7135 |
| EEEE | 19 | GLY | 5.6751 | 5.6751 | 0.0000 |
| EEEE | 20 | GLU | 3.4651 | 0.0024 | 6.2352 |
| EEEE | 21 | ASN | 5.3587 | 3.9397 | 6.7777 |
| EEEE | 22 | VAL | 0.3206 | 0.4678 | 0.1243 |
| EEEE | 23 | THR | 5.2106 | 0.0240 | 12.1261 |
| EEEE | 24 | LEU | 0.2668 | 0.0000 | 0.5335 |
| EEEE | 25 | THR | 4.8755 | 0.0000 | 11.3763 |
| EEEE | 26 | CYS | 0.2422 | 0.3394 | 0.0477 |
| EEEE | 27 | ASN | 7.5792 | 1.5782 | 13.5803 |
| EEEE | 28 | GLY | 7.7171 | 7.7171 | 0.0000 |
| EEEE | 29 | ASN | 17.1451 | 8.1440 | 26.1463 |
| EEEE | 30 | ASN | 14.2079 | 9.6254 | 18.7903 |
| EEEE | 31 | PHE | 18.7547 | 8.6665 | 24.5194 |
| EEEE | 32 | PHE | 7.2539 | 7.0102 | 7.3931 |
| EEEE | 33 | GLU | 17.0855 | 4.7908 | 26.9213 |
| EEEE | 34 | VAL | 10.0735 | 3.1424 | 19.3149 |
| EEEE | 35 | SER | 13.8902 | 2.0792 | 37.5120 |
| EEEE | 36 | SER | 6.8523 | 1.7904 | 16.9760 |
| EEEE | 37 | THR | 0.6763 | 1.1836 | 0.0000 |
| EEEE | 38 | LYS | 8.3619 | 0.3108 | 14.8028 |
| EEEE | 39 | TRP | 0.0083 | 0.0020 | 0.0108 |
| EEEE | 40 | PHE | 3.0042 | 0.0000 | 4.7209 |
| EEEE | 41 | HIS | 3.3299 | 0.3808 | 5.2960 |
| EEEE | 42 | ASN | 6.3452 | 7.0674 | 5.6231 |
| EEEE | 43 | GLY | 9.6662 | 9.6662 | 0.0000 |
| EEEE | 44 | SER | 12.6323 | 3.0497 | 31.7975 |
| EEEE | 45 | LEU | 14.2883 | 6.9452 | 21.6315 |
| EEEE | 46 | SER | 5.9546 | 3.8685 | 10.1268 |
| EEEE | 47 | GLU | 19.8778 | 7.9851 | 29.3920 |
| EEEE | 48 | GLU | 6.2775 | 2.2956 | 9.4630 |
| EEEE | 49 | THR | 11.1492 | 0.0819 | 25.9055 |
| EEEE | 50 | ASN | 3.8263 | 0.9910 | 6.6616 |
| EEEE | 51 | SER | 0.9846 | 0.0000 | 2.9539 |
| EEEE | 52 | SER | 2.8049 | 0.0006 | 8.4134 |
| EEEE | 53 | LEU | 2.6766 | 0.0083 | 5.3450 |
| EEEE | 54 | ASN | 7.1063 | 3.8883 | 10.3243 |
| EEEE | 55 | ILE | 2.1074 | 1.5164 | 2.6984 |
| EEEE | 56 | VAL | 11.4388 | 1.7734 | 24.3260 |
| EEEE | 57 | ASN | 8.9664 | 1.0480 | 16.8848 |
| EEEE | 58 | ALA | 0.3426 | 0.4045 | 0.0949 |
| EEEE | 59 | LYS | 13.5640 | 0.0175 | 24.4012 |
| EEEE | 60 | PHE | 3.4104 | 0.0051 | 5.3562 |
| EEEE | 61 | GLU | 8.6064 | 0.1863 | 15.3424 |
| EEEE | 62 | ASP | 4.2246 | 0.0962 | 8.3531 |
| EEEE | 63 | SER | 0.1134 | 0.0000 | 0.3402 |
| EEEE | 64 | GLY | 0.0266 | 0.0266 | 0.0000 |
| EEEE | 65 | GLU | 4.0549 | 0.0880 | 7.2284 |
| EEEE | 66 | TYR | 0.7832 | 0.0000 | 1.1748 |
| EEEE | 67 | LYS | 3.2565 | 0.0000 | 5.8617 |
| EEEE | 68 | CYS | 0.0003 | 0.0005 | 0.0000 |
| EEEE | 69 | GLN | 3.8454 | 0.0000 | 6.9217 |
| EEEE | 70 | HIS | 2.2450 | 0.2224 | 3.5934 |
| EEEE | 71 | GLN | 5.9169 | 3.6396 | 7.7387 |
| EEEE | 72 | GLN | 7.4965 | 6.0264 | 8.6726 |
| EEEE | 73 | VAL | 6.4019 | 2.4812 | 11.6294 |
| EEEE | 74 | ASN | 7.3226 | 0.8649 | 13.7804 |
| EEEE | 75 | GLU | 9.5795 | 4.4019 | 13.7216 |
| EEEE | 76 | SER | 0.7013 | 1.0519 | 0.0000 |
| EEEE | 77 | GLU | 13.4227 | 1.1013 | 23.2799 |
| EEEE | 78 | PRO | 2.4705 | 1.5274 | 3.7280 |
| EEEE | 79 | VAL | 5.5768 | 0.7997 | 11.9463 |
| EEEE | 80 | TYR | 3.6523 | 1.2328 | 4.8621 |
| EEEE | 81 | LEU | 0.2451 | 0.0000 | 0.4902 |
| EEEE | 82 | GLU | 5.5763 | 0.0256 | 10.0168 |
| EEEE | 83 | VAL | 1.5663 | 2.7355 | 0.0074 |
| EEEE | 84 | PHE | 2.8317 | 0.6685 | 4.0678 |
| EEEE | 85 | SER | 11.1097 | 5.8236 | 21.6819 |
| EEEE | 86 | ASP | 6.3216 | 2.7103 | 9.9328 |
| EEEE | 87 | TRP | 9.2509 | 0.2339 | 12.8578 |
| EEEE | 88 | LEU | 0.0596 | 0.1001 | 0.0191 |
| EEEE | 89 | LEU | 0.1011 | 0.0000 | 0.2022 |
| EEEE | 90 | LEU | 0.0000 | 0.0000 | 0.0000 |
| EEEE | 91 | GLN | 0.2558 | 0.0000 | 0.4604 |
| EEEE | 92 | ALA | 0.0564 | 0.0519 | 0.0745 |
| EEEE | 93 | SER | 4.5837 | 2.9367 | 7.8777 |
| EEEE | 94 | ALA | 8.3906 | 1.2888 | 36.7978 |
| EEEE | 95 | GLU | 4.4296 | 1.5357 | 6.7447 |
| EEEE | 96 | VAL | 4.3010 | 4.6742 | 3.8035 |
| EEEE | 97 | VAL | 1.4250 | 1.0740 | 1.8929 |
| EEEE | 98 | MET | 13.5431 | 0.2108 | 26.8754 |
| EEEE | 99 | GLU | 7.1778 | 4.7822 | 9.0943 |
| EEEE | 100 | GLY | 5.0685 | 5.0685 | 0.0000 |
| EEEE | 101 | GLN | 9.8626 | 0.0125 | 17.7427 |
| EEEE | 102 | PRO | 9.4878 | 1.6105 | 19.9908 |
| EEEE | 103 | LEU | 0.0128 | 0.0246 | 0.0009 |
| EEEE | 104 | PHE | 6.2895 | 0.0000 | 9.8835 |
| EEEE | 105 | LEU | 0.0574 | 0.0715 | 0.0432 |
| EEEE | 106 | ARG | 2.3284 | 0.0000 | 3.6589 |
| EEEE | 107 | CYS | 0.7794 | 1.1691 | 0.0000 |
| EEEE | 108 | HIS | 1.2031 | 0.2846 | 1.8155 |
| EEEE | 109 | GLY | 1.3076 | 1.3076 | 0.0000 |
| EEEE | 110 | TRP | 4.3507 | 0.3127 | 5.9659 |
| EEEE | 111 | ARG | 14.5626 | 6.7438 | 19.0305 |
| EEEE | 112 | ASN | 12.6107 | 3.8715 | 21.3499 |
| EEEE | 113 | TRP | 7.0410 | 3.3592 | 8.5138 |
| EEEE | 114 | ASP | 11.4765 | 1.9258 | 21.0271 |
| EEEE | 115 | VAL | 0.8593 | 0.6604 | 1.1244 |
| EEEE | 116 | TYR | 5.8929 | 0.0005 | 8.8392 |
| EEEE | 117 | LYS | 11.3051 | 0.9380 | 19.5988 |
| EEEE | 118 | VAL | 0.0000 | 0.0000 | 0.0000 |
| EEEE | 119 | ILE | 4.8820 | 0.0000 | 9.7641 |
| EEEE | 120 | TYR | 0.0103 | 0.0000 | 0.0154 |
| EEEE | 121 | TYR | 3.6542 | 0.0005 | 5.4811 |
| EEEE | 122 | LYS | 3.7815 | 0.6097 | 6.3190 |
| EEEE | 123 | ASP | 10.9462 | 6.9829 | 14.9095 |
| EEEE | 124 | GLY | 13.7762 | 13.7762 | 0.0000 |
| EEEE | 125 | GLU | 13.1766 | 0.5548 | 23.2741 |
| EEEE | 126 | ALA | 14.6424 | 5.3245 | 51.9143 |
| EEEE | 127 | LEU | 9.2224 | 5.9305 | 12.5143 |
| EEEE | 128 | LYS | 11.5329 | 1.8633 | 19.2686 |
| EEEE | 129 | TYR | 10.3194 | 5.0683 | 12.9449 |
| EEEE | 130 | TRP | 8.0715 | 0.9722 | 10.9112 |
| EEEE | 131 | TYR | 11.8508 | 1.0518 | 17.2503 |
| EEEE | 132 | GLU | 12.7984 | 2.2087 | 21.2701 |
| EEEE | 133 | ASN | 5.2222 | 5.2458 | 5.1985 |
| EEEE | 134 | HIS | 8.2754 | 1.2222 | 12.9775 |
| EEEE | 135 | ASN | 1.3104 | 0.3410 | 2.2798 |
| EEEE | 136 | ILE | 2.0064 | 1.3737 | 2.6391 |
| EEEE | 137 | SER | 10.1799 | 7.2553 | 16.0292 |
| EEEE | 138 | ILE | 3.5424 | 0.9990 | 6.0858 |
| EEEE | 139 | THR | 16.1230 | 2.4880 | 34.3029 |
| EEEE | 140 | ASN | 6.0914 | 3.7069 | 8.4760 |
| EEEE | 141 | ALA | 0.0000 | 0.0000 | 0.0000 |
| EEEE | 142 | THR | 7.1532 | 0.3267 | 16.2552 |
| EEEE | 143 | VAL | 4.0502 | 1.4721 | 7.4876 |
| EEEE | 144 | GLU | 14.1982 | 4.3093 | 22.1094 |
| EEEE | 145 | ASP | 4.2616 | 0.0294 | 8.4938 |
| EEEE | 146 | SER | 5.0852 | 3.1109 | 9.0339 |
| EEEE | 147 | GLY | 3.2633 | 3.2633 | 0.0000 |
| EEEE | 148 | THR | 5.3711 | 0.0808 | 12.4248 |
| EEEE | 149 | TYR | 0.2123 | 0.0000 | 0.3185 |
| EEEE | 150 | TYR | 3.8241 | 0.0000 | 5.7362 |
| EEEE | 151 | CYS | 0.0000 | 0.0000 | 0.0000 |
| EEEE | 152 | THR | 3.8973 | 0.0053 | 9.0866 |
| EEEE | 153 | GLY | 1.0506 | 1.0506 | 0.0000 |
| EEEE | 154 | LYS | 6.3259 | 0.0465 | 11.3493 |
| EEEE | 155 | VAL | 0.4347 | 0.0000 | 1.0143 |
| EEEE | 156 | TRP | 10.7736 | 3.6761 | 13.6126 |
| EEEE | 157 | GLN | 13.4826 | 3.3463 | 21.5916 |
| EEEE | 158 | LEU | 13.9288 | 0.1999 | 27.6578 |
| EEEE | 159 | ASP | 14.3643 | 5.1303 | 23.5982 |
| EEEE | 160 | TYR | 3.4607 | 2.4956 | 3.9432 |
| EEEE | 161 | GLU | 12.5195 | 4.7567 | 18.7297 |
| EEEE | 162 | SER | 1.0778 | 1.6166 | 0.0000 |
| EEEE | 163 | GLU | 9.3641 | 0.1874 | 16.7054 |
| EEEE | 164 | PRO | 9.7812 | 1.5531 | 20.7519 |
| EEEE | 165 | LEU | 1.6355 | 0.0916 | 3.1794 |
| EEEE | 166 | ASN | 3.8982 | 1.0603 | 6.7362 |
| EEEE | 167 | ILE | 0.5697 | 1.1379 | 0.0016 |
| EEEE | 168 | THR | 2.3606 | 0.0217 | 5.4790 |
| EEEE | 169 | VAL | 0.0074 | 0.0129 | 0.0000 |
| EEEE | 170 | ILE | 2.2300 | 0.0089 | 4.4512 |
| EEEE | 171 | LYS | 14.7618 | 13.0272 | 16.1495 |
| EEEE | 221 | NAG | 12.9978 | 0.0000 | 12.9978 |
| EEEE | 222 | NAG | 20.1629 | 0.0000 | 20.1629 |
| EEEE | 242 | NAG | 8.4007 | 0.0000 | 8.4007 |
| EEEE | 243 | NAG | 8.4488 | 0.0000 | 8.4488 |
| EEEE | 244 | MAN | 16.3142 | 0.0000 | 16.3142 |
| EEEE | 250 | NAG | 15.9751 | 0.0000 | 15.9751 |
| EEEE | 274 | NAG | 18.4789 | 0.0000 | 18.4789 |
| EEEE | 335 | NAG | 14.8589 | 0.0000 | 14.8589 |
| EEEE | 340 | NAG | 17.7265 | 0.0000 | 17.7265 |
| EEEE | 366 | NAG | 11.7893 | 0.0000 | 11.7893 |
| EEEE | 367 | NAG | 18.5598 | 0.0000 | 18.5598 |

TABLE 10
PhFcεRIα₁₋₁₇₂, Form T2, residue exposure
>>>> coordinate set = pent74_11c1.pdb

| segid | resid | resname | access | access-main | access-side |
|---|---|---|---|---|---|
| CCCC | 4 | LYS | 22.5230 | 10.8738 | 31.8424 |
| CCCC | 5 | PRO | 1.1416 | 1.5686 | 0.5722 |
| CCCC | 6 | LYS | 17.1986 | 1.4144 | 29.8259 |
| CCCC | 7 | VAL | 1.6270 | 2.8202 | 0.0362 |
| CCCC | 8 | SER | 8.6366 | 1.9053 | 22.0990 |
| CCCC | 9 | LEU | 4.4395 | 5.4841 | 3.3949 |
| CCCC | 10 | ASN | 12.0444 | 0.9261 | 23.1626 |
| CCCC | 11 | PRO | 7.7510 | 0.4778 | 17.4486 |
| CCCC | 12 | PRO | 10.0046 | 2.2549 | 20.3375 |
| CCCC | 13 | TRP | 1.5672 | 0.1129 | 2.1488 |
| CCCC | 14 | ASN | 3.3834 | 0.3515 | 6.4153 |
| CCCC | 15 | ARG | 1.7214 | 0.0000 | 2.7051 |
| CCCC | 16 | ILE | 0.9799 | 0.0000 | 1.9599 |
| CCCC | 17 | PHE | 0.2972 | 0.0000 | 0.4670 |
| CCCC | 18 | LYS | 11.0480 | 3.8504 | 16.8061 |
| CCCC | 19 | GLY | 5.6439 | 5.6439 | 0.0000 |
| CCCC | 20 | GLU | 3.6350 | 0.0352 | 6.5149 |
| CCCC | 21 | ASN | 5.3939 | 4.1927 | 6.5951 |
| CCCC | 22 | VAL | 0.2954 | 0.4493 | 0.0902 |
| CCCC | 23 | THR | 5.4708 | 0.0934 | 12.6405 |
| CCCC | 24 | LEU | 0.4978 | 0.0000 | 0.9957 |
| CCCC | 25 | THR | 5.1976 | 0.0006 | 12.1271 |
| CCCC | 26 | CYS | 0.3073 | 0.3299 | 0.2621 |
| CCCC | 27 | ASN | 8.5100 | 1.3260 | 15.6940 |
| CCCC | 28 | GLY | 6.8597 | 6.8597 | 0.0000 |
| CCCC | 29 | ASN | 17.2073 | 7.8659 | 26.5486 |
| CCCC | 30 | ASN | 14.4090 | 9.6919 | 19.1261 |
| CCCC | 31 | PHE | 18.6560 | 9.4593 | 23.9112 |
| CCCC | 32 | PHE | 7.3970 | 6.8547 | 7.7068 |
| CCCC | 33 | GLU | 17.4505 | 4.8495 | 27.5314 |
| CCCC | 34 | VAL | 10.1697 | 3.7420 | 18.7399 |
| CCCC | 35 | SER | 14.6634 | 2.2968 | 39.3965 |
| CCCC | 36 | SER | 7.1609 | 1.9487 | 17.5852 |
| CCCC | 37 | THR | 0.7165 | 1.2539 | 0.0000 |
| CCCC | 38 | LYS | 8.8073 | 0.3430 | 15.5788 |
| CCCC | 39 | TRP | 0.0048 | 0.0169 | 0.0000 |
| CCCC | 40 | PHE | 3.0629 | 0.0000 | 4.8131 |
| CCCC | 41 | HIS | 3.2953 | 0.2185 | 5.3465 |
| CCCC | 42 | ASN | 6.4333 | 7.3988 | 5.4678 |
| CCCC | 43 | GLY | 8.7787 | 8.7787 | 0.0000 |
| CCCC | 44 | SER | 12.5780 | 3.2206 | 31.2928 |
| CCCC | 45 | LEU | 14.3835 | 6.5311 | 22.2359 |
| CCCC | 46 | SER | 6.0642 | 4.1760 | 9.8407 |
| CCCC | 47 | GLU | 19.8848 | 8.0573 | 29.3468 |
| CCCC | 48 | GLU | 5.7325 | 2.1429 | 8.6042 |
| CCCC | 49 | THR | 11.1779 | 0.0000 | 26.0818 |
| CCCC | 50 | ASN | 3.7291 | 1.0808 | 6.3774 |
| CCCC | 51 | SER | 1.1927 | 0.0000 | 3.5780 |
| CCCC | 52 | SER | 2.7289 | 0.0000 | 8.1866 |
| CCCC | 53 | LEU | 2.8602 | 0.0189 | 5.7014 |
| CCCC | 54 | ASN | 7.0896 | 4.0346 | 10.1446 |
| CCCC | 55 | ILE | 1.9722 | 1.4947 | 2.4497 |
| CCCC | 56 | VAL | 11.4466 | 1.8119 | 24.2929 |
| CCCC | 57 | ASN | 9.2079 | 1.1203 | 17.2954 |
| CCCC | 58 | ALA | 0.2682 | 0.3352 | 0.0000 |
| CCCC | 59 | LYS | 13.5663 | 0.0799 | 24.3554 |
| CCCC | 60 | PHE | 4.2653 | 0.0614 | 6.6675 |
| CCCC | 61 | GLU | 9.3104 | 0.1429 | 16.6444 |
| CCCC | 62 | ASP | 4.1004 | 0.0929 | 8.1079 |
| CCCC | 63 | SER | 0.1952 | 0.0000 | 0.5857 |
| CCCC | 64 | GLY | 0.0000 | 0.0000 | 0.0000 |
| CCCC | 65 | GLU | 3.3950 | 0.0904 | 6.0387 |
| CCCC | 66 | TYR | 1.0210 | 0.0004 | 1.5313 |
| CCCC | 67 | LYS | 3.2922 | 0.0006 | 5.9255 |
| CCCC | 68 | CYS | 0.0000 | 0.0000 | 0.0000 |
| CCCC | 69 | GLN | 4.0273 | 0.0000 | 7.2491 |
| CCCC | 70 | HIS | 2.4293 | 0.2618 | 3.8743 |
| CCCC | 71 | GLN | 16.1847 | 6.5416 | 23.8992 |
| CCCC | 72 | GLN | 18.7079 | 6.7237 | 28.2952 |
| CCCC | 73 | VAL | 5.9018 | 2.0768 | 11.0019 |
| CCCC | 74 | ASN | 7.6674 | 1.2523 | 14.0824 |
| CCCC | 75 | GLU | 9.5618 | 4.3436 | 13.7363 |
| CCCC | 76 | SER | 0.7453 | 1.1107 | 0.0146 |
| CCCC | 77 | GLU | 13.4902 | 1.2113 | 23.3134 |
| CCCC | 78 | PRO | 3.7570 | 2.4424 | 5.5097 |
| CCCC | 79 | VAL | 6.2786 | 1.0035 | 13.3121 |
| CCCC | 80 | TYR | 4.8276 | 1.6806 | 6.4011 |
| CCCC | 81 | LEU | 0.4499 | 0.0000 | 0.8998 |
| CCCC | 82 | GLU | 6.0083 | 0.0302 | 10.7907 |
| CCCC | 83 | VAL | 1.5038 | 2.6317 | 0.0000 |
| CCCC | 84 | PHE | 2.9396 | 0.6034 | 4.2746 |
| CCCC | 85 | SER | 11.0379 | 5.9825 | 21.1488 |
| CCCC | 86 | ASP | 6.6954 | 2.8722 | 10.5186 |
| CCCC | 87 | TRP | 5.7551 | 0.2523 | 7.9563 |
| CCCC | 88 | LEU | 0.0496 | 0.0992 | 0.0000 |
| CCCC | 89 | LEU | 0.0722 | 0.0000 | 0.1444 |
| CCCC | 90 | LEU | 0.0039 | 0.0006 | 0.0071 |
| CCCC | 91 | GLN | 0.2715 | 0.0000 | 0.4886 |
| CCCC | 92 | ALA | 0.1064 | 0.1238 | 0.0367 |
| CCCC | 93 | SER | 4.5560 | 3.0073 | 7.6533 |
| CCCC | 94 | ALA | 8.2925 | 1.3979 | 35.8708 |
| CCCC | 95 | GLU | 5.1869 | 1.5220 | 8.1188 |
| CCCC | 96 | VAL | 5.3247 | 4.5540 | 6.3524 |
| CCCC | 97 | VAL | 1.5905 | 0.9728 | 2.4141 |
| CCCC | 98 | MET | 14.3166 | 0.0743 | 28.5590 |
| CCCC | 99 | GLU | 7.0891 | 5.0739 | 8.7013 |
| CCCC | 100 | GLY | 5.1879 | 5.1879 | 0.0000 |
| CCCC | 101 | GLN | 9.5976 | 0.0133 | 17.2651 |
| CCCC | 102 | PRO | 9.4229 | 1.5439 | 19.9284 |
| CCCC | 103 | LEU | 0.0333 | 0.0371 | 0.0295 |
| CCCC | 104 | PHE | 6.3516 | 0.0000 | 9.9811 |
| CCCC | 105 | LEU | 0.1059 | 0.0591 | 0.1526 |
| CCCC | 106 | ARG | 2.2520 | 0.0000 | 3.5388 |
| CCCC | 107 | CYS | 0.6406 | 0.9609 | 0.0000 |
| CCCC | 108 | HIS | 1.1793 | 0.2252 | 1.8153 |
| CCCC | 109 | GLY | 1.3114 | 1.3114 | 0.0000 |
| CCCC | 110 | TRP | 4.6295 | 0.3368 | 6.3465 |
| CCCC | 111 | ARG | 13.1248 | 6.9961 | 16.6270 |
| CCCC | 112 | ASN | 12.8011 | 4.3120 | 21.2901 |
| CCCC | 113 | TRP | 6.0437 | 3.3401 | 7.1251 |
| CCCC | 114 | ASP | 11.9344 | 1.8804 | 21.9884 |
| CCCC | 115 | VAL | 0.9151 | 0.7229 | 1.1714 |
| CCCC | 116 | TYR | 5.9569 | 0.0000 | 8.9354 |
| CCCC | 117 | LYS | 11.0444 | 0.6651 | 19.3478 |
| CCCC | 118 | VAL | 0.0000 | 0.0000 | 0.0000 |
| CCCC | 119 | ILE | 4.4790 | 0.0001 | 8.9579 |
| CCCC | 120 | TYR | 0.0043 | 0.0000 | 0.0064 |
| CCCC | 121 | TYR | 3.7210 | 0.0085 | 5.5773 |
| CCCC | 122 | LYS | 4.0141 | 0.9317 | 6.4800 |
| CCCC | 123 | ASP | 10.8032 | 6.5278 | 15.0786 |
| CCCC | 124 | GLY | 14.5419 | 14.5419 | 0.0000 |
| CCCC | 125 | GLU | 12.8310 | 0.4067 | 22.7705 |
| CCCC | 126 | ALA | 14.9558 | 5.5670 | 52.5110 |
| CCCC | 127 | LEU | 9.4777 | 6.3362 | 12.6193 |
| CCCC | 128 | LYS | 11.7644 | 1.7577 | 19.7697 |
| CCCC | 129 | TYR | 10.4250 | 4.9480 | 13.1636 |
| CCCC | 130 | TRP | 8.2395 | 1.0761 | 11.1049 |
| CCCC | 131 | TYR | 12.1293 | 0.8795 | 17.7543 |
| CCCC | 132 | GLU | 12.7495 | 1.9453 | 21.3930 |
| CCCC | 133 | ASN | 5.6282 | 5.4710 | 5.7854 |
| CCCC | 134 | HIS | 8.2353 | 1.4518 | 12.7576 |
| CCCC | 135 | ASN | 1.3610 | 0.3583 | 2.3637 |
| CCCC | 136 | ILE | 2.1395 | 1.3206 | 2.9584 |
| CCCC | 137 | SER | 10.0045 | 7.3132 | 15.3872 |
| CCCC | 138 | ILE | 3.5461 | 0.8905 | 6.2017 |
| CCCC | 139 | THR | 15.6326 | 2.4796 | 33.1700 |
| CCCC | 140 | ASN | 6.4183 | 3.6583 | 9.1784 |
| CCCC | 141 | ALA | 0.0002 | 0.0000 | 0.0009 |
| CCCC | 142 | THR | 7.3418 | 0.3932 | 16.6068 |
| CCCC | 143 | VAL | 5.5574 | 1.0899 | 11.5140 |
| CCCC | 144 | GLU | 14.2578 | 4.3490 | 22.1849 |
| CCCC | 145 | ASP | 4.1649 | 0.0654 | 8.2644 |
| CCCC | 146 | SER | 5.4994 | 3.9946 | 8.5092 |
| CCCC | 147 | GLY | 2.9610 | 2.9610 | 0.0000 |
| CCCC | 148 | THR | 5.8295 | 0.0862 | 13.4873 |
| CCCC | 149 | TYR | 0.2839 | 0.0098 | 0.4210 |
| CCCC | 150 | TYR | 3.8352 | 0.0141 | 5.7458 |
| CCCC | 151 | CYS | 0.0000 | 0.0000 | 0.0000 |
| CCCC | 152 | THR | 3.7535 | 0.0000 | 8.7581 |
| CCCC | 153 | GLY | 1.0847 | 1.0847 | 0.0000 |
| CCCC | 154 | LYS | 6.0253 | 0.0000 | 10.8456 |
| CCCC | 155 | VAL | 0.4796 | 0.0000 | 1.1192 |
| CCCC | 156 | TRP | 5.6483 | 5.3816 | 5.7550 |
| CCCC | 157 | GLN | 13.9764 | 3.1848 | 22.6098 |
| CCCC | 158 | LEU | 9.4587 | 0.2983 | 18.6191 |
| CCCC | 159 | ASP | 14.1094 | 4.9876 | 23.2312 |
| CCCC | 160 | TYR | 2.5046 | 2.3069 | 2.6034 |
| CCCC | 161 | GLU | 13.4023 | 4.9331 | 20.1776 |
| CCCC | 162 | SER | 1.0596 | 1.5893 | 0.0000 |
| CCCC | 163 | GLU | 9.9946 | 0.7061 | 17.4254 |
| CCCC | 164 | PRO | 10.2249 | 1.5777 | 21.7546 |
| CCCC | 165 | LEU | 1.6401 | 0.1628 | 3.1175 |
| CCCC | 166 | ASN | 3.3684 | 1.0069 | 5.7299 |
| CCCC | 167 | ILE | 0.5437 | 1.0633 | 0.0240 |
| CCCC | 168 | THR | 4.2337 | 0.0946 | 9.7525 |
| CCCC | 169 | VAL | 0.1117 | 0.1954 | 0.0000 |
| CCCC | 170 | ILE | 2.4168 | 0.0906 | 4.7430 |
| CCCC | 171 | LYS | 14.4505 | 13.2729 | 15.3926 |
| CCCC | 221 | NAG | 13.3692 | 0.0000 | 13.3692 |
| CCCC | 222 | NAG | 19.4652 | 0.0000 | 19.4652 |
| CCCC | 242 | NAG | 9.4466 | 0.0000 | 9.4466 |
| CCCC | 243 | NAG | 8.1868 | 0.0000 | 8.1868 |
| CCCC | 244 | MAN | 18.7031 | 0.0000 | 18.7031 |
| CCCC | 250 | NAG | 16.1904 | 0.0000 | 16.1904 |
| CCCC | 274 | NAG | 21.9195 | 0.0000 | 21.9195 |
| CCCC | 335 | NAG | 15.0294 | 0.0000 | 15.0294 |
| CCCC | 340 | NAG | 17.5228 | 0.0000 | 17.5228 |
| CCCC | 366 | NAG | 12.1164 | 0.0000 | 12.1164 |
| CCCC | 367 | NAG | 19.5921 | 0.0000 | 19.5921 |
| AAAA | 4 | LYS | 20.9627 | 10.5913 | 29.2599 |
| AAAA | 5 | PRO | 1.1603 | 1.5921 | 0.5846 |
| AAAA | 6 | LYS | 16.7967 | 1.3698 | 29.1382 |
| AAAA | 7 | VAL | 1.6748 | 2.8323 | 0.1316 |
| AAAA | 8 | SER | 8.1802 | 1.8708 | 20.7991 |
| AAAA | 9 | LEU | 4.4389 | 5.4321 | 3.4456 |
| AAAA | 10 | ASN | 12.2932 | 0.9523 | 23.6341 |
| AAAA | 11 | PRO | 7.8292 | 0.4916 | 17.6128 |
| AAAA | 12 | PRO | 9.7721 | 2.2628 | 19.7845 |
| AAAA | 13 | TRP | 1.5676 | 0.0823 | 2.1617 |
| AAAA | 14 | ASN | 3.0526 | 0.3315 | 5.7738 |
| AAAA | 15 | ARG | 1.7626 | 0.0011 | 2.7691 |
| AAAA | 16 | ILE | 0.9627 | 0.0000 | 1.9254 |
| AAAA | 17 | PHE | 0.3249 | 0.0000 | 0.5105 |
| AAAA | 18 | LYS | 10.8420 | 3.4841 | 16.7283 |
| AAAA | 19 | GLY | 5.4381 | 5.4381 | 0.0000 |
| AAAA | 20 | GLU | 3.6790 | 0.0263 | 6.6011 |
| AAAA | 21 | ASN | 5.3743 | 4.2040 | 6.5445 |
| AAAA | 22 | VAL | 0.2940 | 0.4433 | 0.0951 |
| AAAA | 23 | THR | 5.8035 | 0.1030 | 13.4041 |
| AAAA | 24 | LEU | 0.4614 | 0.0000 | 0.9228 |
| AAAA | 25 | THR | 5.1305 | 0.0007 | 11.9704 |
| AAAA | 26 | CYS | 0.2915 | 0.3512 | 0.1723 |
| AAAA | 27 | ASN | 6.2421 | 1.5400 | 10.9443 |
| AAAA | 28 | GLY | 6.9474 | 6.9474 | 0.0000 |
| AAAA | 29 | ASN | 17.1386 | 8.6715 | 25.6057 |
| AAAA | 30 | ASN | 14.4072 | 9.6995 | 19.1150 |
| AAAA | 31 | PHE | 15.4860 | 6.5595 | 20.5869 |
| AAAA | 32 | PHE | 3.7024 | 5.7813 | 2.5144 |
| AAAA | 33 | GLU | 6.0657 | 4.8688 | 7.0232 |
| AAAA | 34 | VAL | 5.5276 | 3.8118 | 7.8153 |
| AAAA | 35 | SER | 14.1613 | 2.3658 | 37.7522 |
| AAAA | 36 | SER | 7.1159 | 1.7815 | 17.7848 |
| AAAA | 37 | THR | 0.7225 | 1.2641 | 0.0004 |
| AAAA | 38 | LYS | 8.7294 | 0.3364 | 15.4439 |
| AAAA | 39 | TRP | 0.0092 | 0.0322 | 0.0000 |
| AAAA | 40 | PHE | 3.1015 | 0.0000 | 4.8738 |
| AAAA | 41 | HIS | 3.4263 | 0.4703 | 5.3969 |
| AAAA | 42 | ASN | 6.4271 | 7.3557 | 5.4985 |
| AAAA | 43 | GLY | 8.7869 | 8.7869 | 0.0000 |
| AAAA | 44 | SER | 12.6493 | 3.0960 | 31.7559 |
| AAAA | 45 | LEU | 14.5777 | 6.7914 | 22.3640 |
| AAAA | 46 | SER | 5.5814 | 3.9481 | 8.8482 |
| AAAA | 47 | GLU | 19.7555 | 7.9429 | 29.2055 |
| AAAA | 48 | GLU | 5.9957 | 2.0924 | 9.1182 |
| AAAA | 49 | THR | 11.1755 | 0.0000 | 26.0762 |
| AAAA | 50 | ASN | 3.7477 | 1.0911 | 6.4042 |
| AAAA | 51 | SER | 1.1636 | 0.0000 | 3.4907 |
| AAAA | 52 | SER | 2.7526 | 0.0004 | 8.2570 |
| AAAA | 53 | LEU | 2.9437 | 0.0016 | 5.8859 |
| AAAA | 54 | ASN | 7.0747 | 3.8236 | 10.3258 |
| AAAA | 55 | ILE | 1.9632 | 1.5156 | 2.4109 |
| AAAA | 56 | VAL | 11.4314 | 1.7967 | 24.2777 |
| AAAA | 57 | ASN | 8.8194 | 1.1924 | 16.4463 |
| AAAA | 58 | ALA | 0.3818 | 0.4773 | 0.0000 |
| AAAA | 59 | LYS | 14.0268 | 0.1179 | 25.1539 |
| AAAA | 60 | PHE | 4.2543 | 0.0600 | 6.6511 |
| AAAA | 61 | GLU | 9.3832 | 0.1346 | 16.7820 |
| AAAA | 62 | ASP | 4.0523 | 0.0770 | 8.0275 |
| AAAA | 63 | SER | 0.1601 | 0.0000 | 0.4802 |
| AAAA | 64 | GLY | 0.0003 | 0.0003 | 0.0000 |
| AAAA | 65 | GLU | 7.8567 | 0.0750 | 14.0821 |
| AAAA | 66 | TYR | 1.0215 | 0.0000 | 1.5322 |
| AAAA | 67 | LYS | 3.3027 | 0.0000 | 5.9449 |
| AAAA | 68 | CYS | 0.0000 | 0.0000 | 0.0000 |
| AAAA | 69 | GLN | 3.9650 | 0.0000 | 7.1371 |
| AAAA | 70 | HIS | 1.3538 | 0.2724 | 2.0747 |
| AAAA | 71 | GLN | 16.3949 | 6.5138 | 24.2997 |
| AAAA | 72 | GLN | 18.9827 | 6.9646 | 28.5973 |
| AAAA | 73 | VAL | 4.9867 | 2.0211 | 8.9408 |
| AAAA | 74 | ASN | 7.4791 | 1.0651 | 13.8931 |
| AAAA | 75 | GLU | 9.6144 | 4.2579 | 13.8996 |
| AAAA | 76 | SER | 0.6674 | 0.9903 | 0.0214 |
| AAAA | 77 | GLU | 15.9242 | 1.2854 | 27.6353 |
| AAAA | 78 | PRO | 8.1677 | 4.8944 | 12.5320 |
| AAAA | 79 | VAL | 6.4525 | 1.5909 | 12.9345 |
| AAAA | 80 | TYR | 7.2923 | 1.8827 | 9.9971 |
| AAAA | 81 | LEU | 0.4642 | 0.0000 | 0.9283 |
| AAAA | 82 | GLU | 6.0060 | 0.0321 | 10.7852 |
| AAAA | 83 | VAL | 1.5422 | 2.6988 | 0.0000 |
| AAAA | 84 | PHE | 2.9572 | 0.6545 | 4.2730 |
| AAAA | 85 | SER | 10.8486 | 6.1560 | 20.2338 |
| AAAA | 86 | ASP | 6.1844 | 2.2433 | 10.1256 |
| AAAA | 87 | TRP | 9.3655 | 0.2163 | 13.0252 |
| AAAA | 88 | LEU | 0.0504 | 0.1008 | 0.0000 |
| AAAA | 89 | LEU | 0.1469 | 0.0000 | 0.2937 |
| AAAA | 90 | LEU | 0.0016 | 0.0007 | 0.0025 |
| AAAA | 91 | GLN | 0.2732 | 0.0066 | 0.4865 |
| AAAA | 92 | ALA | 0.0432 | 0.0426 | 0.0458 |
| AAAA | 93 | SER | 4.4502 | 2.9733 | 7.4040 |
| AAAA | 94 | ALA | 8.7362 | 1.3963 | 38.0959 |
| AAAA | 95 | GLU | 7.1768 | 1.5655 | 11.6658 |
| AAAA | 96 | VAL | 13.5692 | 4.6624 | 25.4450 |
| AAAA | 97 | VAL | 1.5462 | 0.9380 | 2.3571 |
| AAAA | 98 | MET | 14.3960 | 0.0702 | 28.7219 |
| AAAA | 99 | GLU | 7.3826 | 4.6030 | 9.6063 |
| AAAA | 100 | GLY | 4.9867 | 4.9867 | 0.0000 |
| AAAA | 101 | GLN | 9.6618 | 0.0164 | 17.3782 |
| AAAA | 102 | PRO | 9.4982 | 1.5257 | 20.1283 |
| AAAA | 103 | LEU | 0.0170 | 0.0095 | 0.0245 |
| AAAA | 104 | PHE | 6.3600 | 0.0000 | 9.9943 |
| AAAA | 105 | LEU | 0.0964 | 0.0678 | 0.1250 |
| AAAA | 106 | ARG | 2.2271 | 0.0002 | 3.4997 |
| AAAA | 107 | CYS | 0.6410 | 0.9615 | 0.0000 |
| AAAA | 108 | HIS | 1.1779 | 0.2061 | 1.8257 |
| AAAA | 109 | GLY | 1.2835 | 1.2835 | 0.0000 |
| AAAA | 110 | TRP | 4.3852 | 0.3446 | 6.0014 |
| AAAA | 111 | ARG | 14.5965 | 6.9788 | 18.9495 |
| AAAA | 112 | ASN | 13.1375 | 4.4344 | 21.8406 |
| AAAA | 113 | TRP | 7.1680 | 3.4865 | 8.6407 |
| AAAA | 114 | ASP | 11.7831 | 1.8946 | 21.6716 |
| AAAA | 115 | VAL | 0.9352 | 0.7629 | 1.1650 |
| AAAA | 116 | TYR | 5.3542 | 0.0000 | 8.0313 |
| AAAA | 117 | LYS | 7.2506 | 0.7461 | 12.4542 |
| AAAA | 118 | VAL | 0.0000 | 0.0000 | 0.0000 |
| AAAA | 119 | ILE | 1.6994 | 0.0000 | 3.3988 |
| AAAA | 120 | TYR | 0.0315 | 0.0007 | 0.0469 |
| AAAA | 121 | TYR | 2.9781 | 0.0007 | 4.4667 |
| AAAA | 122 | LYS | 3.9855 | 0.7635 | 6.5630 |
| AAAA | 123 | ASP | 10.6844 | 6.3101 | 15.0587 |
| AAAA | 124 | GLY | 14.6459 | 14.6459 | 0.0000 |
| AAAA | 125 | GLU | 7.8188 | 0.3898 | 13.7619 |
| AAAA | 126 | ALA | 2.0714 | 1.0926 | 5.9866 |
| AAAA | 127 | LEU | 5.8795 | 1.4447 | 10.3142 |
| AAAA | 128 | LYS | 11.0255 | 0.1820 | 19.7002 |
| AAAA | 129 | TYR | 5.6220 | 4.2110 | 6.3275 |
| AAAA | 130 | TRP | 6.9643 | 1.0858 | 9.3157 |
| AAAA | 131 | TYR | 2.7608 | 0.9482 | 3.6671 |
| AAAA | 132 | GLU | 9.8508 | 1.9760 | 16.1507 |
| AAAA | 133 | ASN | 5.7009 | 5.5383 | 5.8635 |
| AAAA | 134 | HIS | 8.0039 | 1.5080 | 12.3344 |
| AAAA | 135 | ASN | 1.3397 | 0.3105 | 2.3689 |
| AAAA | 136 | ILE | 2.1821 | 1.3384 | 3.0258 |
| AAAA | 137 | SER | 9.9955 | 7.3477 | 15.2912 |
| AAAA | 138 | ILE | 3.4842 | 0.8752 | 6.0933 |
| AAAA | 139 | THR | 15.7464 | 2.4744 | 33.4424 |
| AAAA | 140 | ASN | 6.4552 | 3.7317 | 9.1786 |
| AAAA | 141 | ALA | 0.0000 | 0.0000 | 0.0000 |
| AAAA | 142 | THR | 7.4724 | 0.4042 | 16.8967 |
| AAAA | 143 | VAL | 7.7469 | 1.0561 | 16.6680 |
| AAAA | 144 | GLU | 14.1113 | 4.3219 | 21.9429 |
| AAAA | 145 | ASP | 4.3240 | 0.0500 | 8.5980 |
| AAAA | 146 | SER | 5.4853 | 4.0599 | 8.3361 |
| AAAA | 147 | GLY | 2.8492 | 2.8492 | 0.0000 |
| AAAA | 148 | THR | 5.7830 | 0.0884 | 13.3757 |
| AAAA | 149 | TYR | 0.2720 | 0.0018 | 0.4071 |
| AAAA | 150 | TYR | 3.9253 | 0.0098 | 5.8831 |
| AAAA | 151 | CYS | 0.0000 | 0.0000 | 0.0000 |
| AAAA | 152 | THR | 3.7350 | 0.0000 | 8.7151 |
| AAAA | 153 | GLY | 1.0268 | 1.0268 | 0.0000 |
| AAAA | 154 | LYS | 4.9160 | 0.0084 | 8.8421 |
| AAAA | 155 | VAL | 0.4691 | 0.0000 | 1.0946 |
| AAAA | 156 | TRP | 11.7056 | 5.3116 | 14.2632 |
| AAAA | 157 | GLN | 9.2515 | 3.3242 | 13.9933 |
| AAAA | 158 | LEU | 13.8847 | 0.2727 | 27.4967 |
| AAAA | 159 | ASP | 9.2336 | 3.2139 | 15.2533 |
| AAAA | 160 | TYR | 3.5492 | 2.3626 | 4.1425 |
| AAAA | 161 | GLU | 11.2704 | 5.0419 | 16.2531 |
| AAAA | 162 | SER | 1.1125 | 1.6687 | 0.0000 |
| AAAA | 163 | GLU | 9.7709 | 0.4328 | 17.2413 |
| AAAA | 164 | PRO | 10.3588 | 1.5740 | 22.0717 |
| AAAA | 165 | LEU | 1.6439 | 0.1655 | 3.1223 |
| AAAA | 166 | ASN | 4.0686 | 1.1039 | 7.0332 |
| AAAA | 167 | ILE | 0.5158 | 1.0240 | 0.0075 |
| AAAA | 168 | THR | 10.0323 | 0.1297 | 23.2358 |
| AAAA | 169 | VAL | 0.5330 | 0.8959 | 0.0491 |
| AAAA | 170 | ILE | 10.6523 | 1.5082 | 19.7963 |
| AAAA | 171 | LYS | 20.1585 | 15.5532 | 23.8428 |
| AAAA | 221 | NAG | 13.2449 | 0.0000 | 13.2449 |
| AAAA | 222 | NAG | 19.9892 | 0.0000 | 19.9892 |
| AAAA | 242 | NAG | 9.9407 | 0.0000 | 9.9407 |
| AAAA | 243 | NAG | 9.4600 | 0.0000 | 9.4600 |
| AAAA | 244 | MAN | 18.6631 | 0.0000 | 18.6631 |
| AAAA | 250 | NAG | 16.3080 | 0.0000 | 16.3080 |
| AAAA | 274 | NAG | 21.8749 | 0.0000 | 21.8749 |
| AAAA | 335 | NAG | 15.0157 | 0.0000 | 15.0157 |
| AAAA | 340 | NAG | 17.2280 | 0.0000 | 17.2280 |
| AAAA | 366 | NAG | 14.4545 | 0.0000 | 14.4545 |
| AAAA | 367 | NAG | 20.9042 | 0.0000 | 20.9042 |
| BBBB | 4 | LYS | 22.4434 | 10.6722 | 31.8604 |
| BBBB | 5 | PRO | 1.1609 | 1.5819 | 0.5996 |
| BBBB | 6 | LYS | 16.5842 | 1.4102 | 28.7235 |
| BBBB | 7 | VAL | 1.6862 | 2.8240 | 0.1692 |
| BBBB | 8 | SER | 8.2982 | 1.8668 | 21.1609 |
| BBBB | 9 | LEU | 4.4129 | 5.4051 | 3.4206 |
| BBBB | 10 | ASN | 11.9525 | 0.9175 | 22.9875 |
| BBBB | 11 | PRO | 7.8250 | 0.4752 | 17.6248 |
| BBBB | 12 | PRO | 10.1980 | 2.1957 | 20.8677 |
| BBBB | 13 | TRP | 1.5849 | 0.0977 | 2.1798 |
| BBBB | 14 | ASN | 3.3380 | 0.3318 | 6.3443 |
| BBBB | 15 | ARG | 1.7418 | 0.0000 | 2.7372 |
| BBBB | 16 | ILE | 0.9354 | 0.0001 | 1.8707 |
| BBBB | 17 | PHE | 0.3221 | 0.0000 | 0.5062 |
| BBBB | 18 | LYS | 11.1312 | 3.8205 | 16.9798 |
| BBBB | 19 | GLY | 5.3141 | 5.3141 | 0.0000 |
| BBBB | 20 | GLU | 3.5506 | 0.0401 | 6.3590 |
| BBBB | 21 | ASN | 5.3304 | 4.1079 | 6.5530 |
| BBBB | 22 | VAL | 0.2845 | 0.4466 | 0.0683 |
| BBBB | 23 | THR | 5.5150 | 0.0841 | 12.7561 |
| BBBB | 24 | LEU | 0.4740 | 0.0000 | 0.9480 |
| BBBB | 25 | THR | 5.1331 | 0.0000 | 11.9772 |
| BBBB | 26 | CYS | 0.2892 | 0.3174 | 0.2327 |
| BBBB | 27 | ASN | 6.4305 | 1.5210 | 11.3399 |
| BBBB | 28 | GLY | 7.2421 | 7.2421 | 0.0000 |
| BBBB | 29 | ASN | 17.3886 | 8.6086 | 26.1686 |
| BBBB | 30 | ASN | 14.2633 | 10.0072 | 18.5194 |
| BBBB | 31 | PHE | 18.8528 | 9.5505 | 24.1684 |
| BBBB | 32 | PHE | 6.5037 | 6.5617 | 6.4706 |
| BBBB | 33 | GLU | 12.9342 | 4.8992 | 19.3623 |
| BBBB | 34 | VAL | 8.3666 | 3.8085 | 14.4440 |
| BBBB | 35 | SER | 14.2536 | 2.2580 | 38.2448 |
| BBBB | 36 | SER | 7.1333 | 1.8956 | 17.6087 |
| BBBB | 37 | THR | 0.7187 | 1.2577 | 0.0000 |
| BBBB | 38 | LYS | 8.8478 | 0.3137 | 15.6751 |
| BBBB | 39 | TRP | 0.0155 | 0.0288 | 0.0102 |
| BBBB | 40 | PHE | 3.1021 | 0.0000 | 4.8747 |
| BBBB | 41 | HIS | 3.3964 | 0.4181 | 5.3819 |
| BBBB | 42 | ASN | 4.3570 | 5.3484 | 3.3656 |
| BBBB | 43 | GLY | 8.6389 | 8.6389 | 0.0000 |
| BBBB | 44 | SER | 12.5229 | 3.2589 | 31.0508 |
| BBBB | 45 | LEU | 14.4289 | 6.6263 | 22.2316 |
| BBBB | 46 | SER | 6.1946 | 4.5349 | 9.5141 |
| BBBB | 47 | GLU | 19.7813 | 8.0788 | 29.1432 |
| BBBB | 48 | GLU | 5.7938 | 2.2706 | 8.6124 |
| BBBB | 49 | THR | 11.0771 | 0.0000 | 25.8466 |
| BBBB | 50 | ASN | 3.6499 | 1.0731 | 6.2267 |
| BBBB | 51 | SER | 1.1954 | 0.0005 | 3.5851 |
| BBBB | 52 | SER | 2.7282 | 0.0002 | 8.1843 |
| BBBB | 53 | LEU | 2.6801 | 0.0033 | 5.3570 |
| BBBB | 54 | ASN | 7.0535 | 4.0402 | 10.0668 |
| BBBB | 55 | ILE | 1.9823 | 1.5180 | 2.4466 |
| BBBB | 56 | VAL | 11.5628 | 1.8317 | 24.5377 |
| BBBB | 57 | ASN | 8.8810 | 1.1220 | 16.6400 |
| BBBB | 58 | ALA | 0.3193 | 0.3991 | 0.0000 |
| BBBB | 59 | LYS | 13.7310 | 0.1005 | 24.6353 |
| BBBB | 60 | PHE | 4.2410 | 0.1526 | 6.5772 |
| BBBB | 61 | GLU | 9.1821 | 0.1249 | 16.4278 |
| BBBB | 62 | ASP | 4.1444 | 0.0742 | 8.2145 |
| BBBB | 63 | SER | 0.1404 | 0.0003 | 0.4207 |
| BBBB | 64 | GLY | 0.0115 | 0.0115 | 0.0000 |
| BBBB | 65 | GLU | 7.8724 | 0.0613 | 14.1212 |
| BBBB | 66 | TYR | 1.0150 | 0.0106 | 1.5171 |
| BBBB | 67 | LYS | 3.3196 | 0.0000 | 5.9752 |
| BBBB | 68 | CYS | 0.0000 | 0.0000 | 0.0000 |
| BBBB | 69 | GLN | 4.0029 | 0.0000 | 7.2052 |
| BBBB | 70 | HIS | 1.6317 | 0.2424 | 2.5580 |
| BBBB | 71 | GLN | 13.5234 | 6.2391 | 19.3508 |
| BBBB | 72 | GLN | 13.7415 | 5.5049 | 20.3307 |
| BBBB | 73 | VAL | 3.0796 | 1.9941 | 4.5269 |
| BBBB | 74 | ASN | 7.4362 | 0.9858 | 13.8867 |
| BBBB | 75 | GLU | 9.6630 | 4.3598 | 13.9056 |
| BBBB | 76 | SER | 0.6085 | 0.9051 | 0.0153 |
| BBBB | 77 | GLU | 15.8267 | 1.2423 | 27.4942 |
| BBBB | 78 | PRO | 8.2618 | 4.8501 | 12.8107 |
| BBBB | 79 | VAL | 6.6038 | 1.6215 | 13.2470 |
| BBBB | 80 | TYR | 7.2918 | 1.9227 | 9.9763 |
| BBBB | 81 | LEU | 0.4359 | 0.0011 | 0.8708 |
| BBBB | 82 | GLU | 5.5353 | 0.0340 | 9.9364 |
| BBBB | 83 | VAL | 1.5227 | 2.6648 | 0.0000 |
| BBBB | 84 | PHE | 2.9287 | 0.6127 | 4.2522 |
| BBBB | 85 | SER | 11.1963 | 6.1246 | 21.3396 |
| BBBB | 86 | ASP | 6.4970 | 2.8341 | 10.1599 |
| BBBB | 87 | TRP | 9.3025 | 0.2173 | 12.9366 |
| BBBB | 88 | LEU | 0.0466 | 0.0933 | 0.0000 |
| BBBB | 89 | LEU | 0.1325 | 0.0000 | 0.2650 |
| BBBB | 90 | LEU | 0.0000 | 0.0000 | 0.0000 |
| BBBB | 91 | GLN | 0.2715 | 0.0001 | 0.4887 |
| BBBB | 92 | ALA | 0.1140 | 0.1208 | 0.0870 |
| BBBB | 93 | SER | 4.3701 | 3.0355 | 7.0392 |
| BBBB | 94 | ALA | 8.8274 | 1.4714 | 38.2514 |
| BBBB | 95 | GLU | 7.1968 | 1.4526 | 11.7922 |
| BBBB | 96 | VAL | 13.4385 | 4.5929 | 25.2325 |
| BBBB | 97 | VAL | 1.5907 | 0.9613 | 2.4301 |
| BBBB | 98 | MET | 14.3698 | 0.0738 | 28.6658 |
| BBBB | 99 | GLU | 7.3281 | 4.9392 | 9.2392 |
| BBBB | 100 | GLY | 5.2577 | 5.2577 | 0.0000 |
| BBBB | 101 | GLN | 9.4091 | 0.0245 | 16.9168 |
| BBBB | 102 | PRO | 9.6291 | 1.5760 | 20.3666 |
| BBBB | 103 | LEU | 0.0129 | 0.0053 | 0.0206 |
| BBBB | 104 | PHE | 6.1594 | 0.0000 | 9.6790 |
| BBBB | 105 | LEU | 0.0613 | 0.0533 | 0.0693 |
| BBBB | 106 | ARG | 2.2836 | 0.0000 | 3.5885 |
| BBBB | 107 | CYS | 0.6725 | 1.0088 | 0.0000 |
| BBBB | 108 | HIS | 1.1253 | 0.2267 | 1.7244 |
| BBBB | 109 | GLY | 1.2775 | 1.2775 | 0.0000 |
| BBBB | 110 | TRP | 4.4509 | 0.3060 | 6.1089 |
| BBBB | 111 | ARG | 14.4604 | 6.9977 | 18.7248 |
| BBBB | 112 | ASN | 13.1855 | 4.3879 | 21.9831 |
| BBBB | 113 | TRP | 7.1019 | 3.5603 | 8.5186 |
| BBBB | 114 | ASP | 11.9934 | 2.2124 | 21.7745 |
| BBBB | 115 | VAL | 0.9738 | 0.8493 | 1.1398 |
| BBBB | 116 | TYR | 5.9699 | 0.0003 | 8.9548 |
| BBBB | 117 | LYS | 11.3675 | 0.6641 | 19.9301 |
| BBBB | 118 | VAL | 0.0000 | 0.0000 | 0.0000 |
| BBBB | 119 | ILE | 4.4231 | 0.0000 | 8.8463 |
| BBBB | 120 | TYR | 0.0060 | 0.0000 | 0.0090 |
| BBBB | 121 | TYR | 3.5477 | 0.0003 | 5.3215 |
| BBBB | 122 | LYS | 3.9640 | 0.7211 | 6.5584 |
| BBBB | 123 | ASP | 10.6309 | 6.1402 | 15.1215 |
| BBBB | 124 | GLY | 14.3673 | 14.3673 | 0.0000 |
| BBBB | 125 | GLU | 13.1648 | 0.4047 | 23.3729 |
| BBBB | 126 | ALA | 14.7406 | 5.6401 | 51.1429 |
| BBBB | 127 | LEU | 9.0669 | 6.2840 | 11.8498 |
| BBBB | 128 | LYS | 11.7318 | 1.7962 | 19.6802 |
| BBBB | 129 | TYR | 10.3977 | 5.0068 | 13.0932 |
| BBBB | 130 | TRP | 8.0404 | 1.1151 | 10.8105 |
| BBBB | 131 | TYR | 12.1527 | 0.8656 | 17.7962 |
| BBBB | 132 | GLU | 12.6929 | 2.0269 | 21.2256 |
| BBBB | 133 | ASN | 5.5740 | 5.4313 | 5.7167 |
| BBBB | 134 | HIS | 8.2722 | 1.4837 | 12.7979 |
| BBBB | 135 | ASN | 1.3495 | 0.3379 | 2.3611 |
| BBBB | 136 | ILE | 2.1772 | 1.2842 | 3.0701 |
| BBBB | 137 | SER | 10.0581 | 7.4531 | 15.2681 |
| BBBB | 138 | ILE | 3.4589 | 0.9040 | 6.0137 |
| BBBB | 139 | THR | 15.5520 | 2.5023 | 32.9516 |
| BBBB | 140 | ASN | 6.4140 | 3.6881 | 9.1399 |
| BBBB | 141 | ALA | 0.0003 | 0.0003 | 0.0000 |
| BBBB | 142 | THR | 6.5774 | 0.3047 | 14.9409 |
| BBBB | 143 | VAL | 7.8299 | 1.0449 | 16.8764 |
| BBBB | 144 | GLU | 14.1982 | 4.2567 | 22.1514 |
| BBBB | 145 | ASP | 4.3806 | 0.0466 | 8.7146 |
| BBBB | 146 | SER | 5.5841 | 4.1811 | 8.3902 |
| BBBB | 147 | GLY | 2.9485 | 2.9485 | 0.0000 |
| BBBB | 148 | THR | 5.5009 | 0.1035 | 12.6974 |
| BBBB | 149 | TYR | 0.2416 | 0.0180 | 0.3534 |
| BBBB | 150 | TYR | 3.8781 | 0.0000 | 5.8171 |
| BBBB | 151 | CYS | 0.0000 | 0.0000 | 0.0000 |
| BBBB | 152 | THR | 3.8579 | 0.0000 | 9.0018 |
| BBBB | 153 | GLY | 1.0286 | 1.0286 | 0.0000 |
| BBBB | 154 | LYS | 6.0883 | 0.0037 | 10.9561 |
| BBBB | 155 | VAL | 0.4604 | 0.0000 | 1.0742 |
| BBBB | 156 | TRP | 11.6843 | 5.2412 | 14.2615 |
| BBBB | 157 | GLN | 14.2169 | 3.2783 | 22.9678 |
| BBBB | 158 | LEU | 13.8111 | 0.3293 | 27.2930 |
| BBBB | 159 | ASP | 14.3170 | 4.9359 | 23.6980 |
| BBBB | 160 | TYR | 3.5010 | 2.3021 | 4.1004 |
| BBBB | 161 | GLU | 13.3785 | 5.0194 | 20.0658 |
| BBBB | 162 | SER | 1.1216 | 1.6824 | 0.0000 |
| BBBB | 163 | GLU | 9.8626 | 0.5373 | 17.3229 |
| BBBB | 164 | PRO | 10.0802 | 1.5190 | 21.4953 |
| BBBB | 165 | LEU | 1.6748 | 0.1571 | 3.1924 |
| BBBB | 166 | ASN | 4.0864 | 1.0418 | 7.1311 |
| BBBB | 167 | ILE | 0.5430 | 1.0598 | 0.0262 |
| BBBB | 168 | THR | 9.9575 | 0.1056 | 23.0934 |
| BBBB | 169 | VAL | 0.5482 | 0.9594 | 0.0000 |
| BBBB | 170 | ILE | 10.7170 | 1.5437 | 19.8904 |
| BBBB | 171 | LYS | 20.1725 | 15.3137 | 24.0596 |
| BBBB | 221 | NAG | 13.1802 | 0.0000 | 13.1802 |
| BBBB | 222 | NAG | 20.4108 | 0.0000 | 20.4108 |
| BBBB | 242 | NAG | 7.5051 | 0.0000 | 7.5051 |
| BBBB | 243 | NAG | 7.8194 | 0.0000 | 7.8194 |
| BBBB | 244 | MAN | 18.6420 | 0.0000 | 18.6420 |
| BBBB | 250 | NAG | 16.2628 | 0.0000 | 16.2628 |
| BBBB | 274 | NAG | 21.8856 | 0.0000 | 21.8856 |
| BBBB | 335 | NAG | 14.8369 | 0.0000 | 14.8369 |
| BBBB | 340 | NAG | 17.4016 | 0.0000 | 17.4016 |
| BBBB | 366 | NAG | 14.6038 | 0.0000 | 14.6038 |
| BBBB | 367 | NAG | 21.0874 | 0.0000 | 21.0874 |
| DDDD | 4 | LYS | 22.1244 | 10.4757 | 31.4434 |
| DDDD | 5 | PRO | 1.1536 | 1.5927 | 0.5680 |
| DDDD | 6 | LYS | 17.0471 | 1.3682 | 29.5902 |
| DDDD | 7 | VAL | 1.6664 | 2.8596 | 0.0755 |
| DDDD | 8 | SER | 8.3148 | 1.8918 | 21.1607 |
| DDDD | 9 | LEU | 4.3290 | 5.2653 | 3.3927 |
| DDDD | 10 | ASN | 12.0081 | 0.9613 | 23.0549 |
| DDDD | 11 | PRO | 8.1330 | 0.4813 | 18.3353 |
| DDDD | 12 | PRO | 10.2920 | 2.3028 | 20.9442 |
| DDDD | 13 | TRP | 1.5549 | 0.0701 | 2.1488 |
| DDDD | 14 | ASN | 3.1974 | 0.3117 | 6.0832 |
| DDDD | 15 | ARG | 1.7368 | 0.0000 | 2.7293 |
| DDDD | 16 | ILE | 0.9559 | 0.0003 | 1.9114 |
| DDDD | 17 | PHE | 0.3209 | 0.0000 | 0.5043 |
| DDDD | 18 | LYS | 10.9142 | 3.4868 | 16.8562 |
| DDDD | 19 | GLY | 5.8859 | 5.8859 | 0.0000 |
| DDDD | 20 | GLU | 3.5184 | 0.0001 | 6.3330 |
| DDDD | 21 | ASN | 5.3913 | 4.1153 | 6.6672 |
| DDDD | 22 | VAL | 0.2777 | 0.4369 | 0.0655 |
| DDDD | 23 | THR | 5.8162 | 0.1076 | 13.4277 |
| DDDD | 24 | LEU | 0.4623 | 0.0006 | 0.9240 |
| DDDD | 25 | THR | 5.1763 | 0.0000 | 12.0779 |
| DDDD | 26 | CYS | 0.2817 | 0.3216 | 0.2020 |
| DDDD | 27 | ASN | 8.2286 | 1.5680 | 14.8891 |
| DDDD | 28 | GLY | 7.4834 | 7.4834 | 0.0000 |
| DDDD | 29 | ASN | 17.4431 | 8.5265 | 26.3597 |
| DDDD | 30 | ASN | 14.3278 | 10.0224 | 18.6333 |
| DDDD | 31 | PHE | 18.8219 | 8.9687 | 24.4524 |
| DDDD | 32 | PHE | 7.0915 | 6.7608 | 7.2806 |
| DDDD | 33 | GLU | 17.4529 | 5.1261 | 27.3143 |
| DDDD | 34 | VAL | 10.0069 | 3.7179 | 18.3922 |
| DDDD | 35 | SER | 14.6436 | 2.2454 | 39.4399 |
| DDDD | 36 | SER | 7.1471 | 1.9242 | 17.5931 |
| DDDD | 37 | THR | 0.7333 | 1.2833 | 0.0000 |
| DDDD | 38 | LYS | 8.8058 | 0.3191 | 15.5951 |
| DDDD | 39 | TRP | 0.0167 | 0.0569 | 0.0007 |
| DDDD | 40 | PHE | 3.0699 | 0.0000 | 4.8241 |
| DDDD | 41 | HIS | 3.3244 | 0.1639 | 5.4315 |
| DDDD | 42 | ASN | 6.2564 | 7.1984 | 5.3143 |
| DDDD | 43 | GLY | 8.8245 | 8.8245 | 0.0000 |
| DDDD | 44 | SER | 12.8423 | 3.1760 | 32.1749 |
| DDDD | 45 | LEU | 14.8050 | 7.3234 | 22.2866 |
| DDDD | 46 | SER | 6.2461 | 4.5119 | 9.7144 |
| DDDD | 47 | GLU | 19.8403 | 7.8973 | 29.3948 |
| DDDD | 48 | GLU | 5.7613 | 2.1656 | 8.6379 |
| DDDD | 49 | THR | 10.9341 | 0.0000 | 25.5128 |
| DDDD | 50 | ASN | 3.7321 | 1.0782 | 6.3860 |
| DDDD | 51 | SER | 1.1905 | 0.0000 | 3.5714 |
| DDDD | 52 | SER | 2.7666 | 0.0003 | 8.2993 |
| DDDD | 53 | LEU | 2.7642 | 0.0050 | 5.5234 |
| DDDD | 54 | ASN | 7.1762 | 4.1170 | 10.2353 |
| DDDD | 55 | ILE | 1.9872 | 1.4615 | 2.5130 |
| DDDD | 56 | VAL | 11.4848 | 1.8199 | 24.3713 |
| DDDD | 57 | ASN | 9.2650 | 1.2272 | 17.3029 |
| DDDD | 58 | ALA | 0.3308 | 0.4134 | 0.0000 |
| DDDD | 59 | LYS | 13.7555 | 0.1261 | 24.6591 |
| DDDD | 60 | PHE | 4.2948 | 0.3462 | 6.5511 |
| DDDD | 61 | GLU | 9.1485 | 0.1440 | 16.3520 |
| DDDD | 62 | ASP | 4.0350 | 0.0620 | 8.0080 |
| DDDD | 63 | SER | 0.1959 | 0.0000 | 0.5877 |
| DDDD | 64 | GLY | 0.0065 | 0.0065 | 0.0000 |
| DDDD | 65 | GLU | 7.8394 | 0.0396 | 14.0791 |
| DDDD | 66 | TYR | 1.0148 | 0.0000 | 1.5222 |
| DDDD | 67 | LYS | 3.3208 | 0.0000 | 5.9774 |
| DDDD | 68 | CYS | 0.0002 | 0.0000 | 0.0005 |
| DDDD | 69 | GLN | 3.9726 | 0.0000 | 7.1507 |
| DDDD | 70 | HIS | 2.6810 | 0.2742 | 4.2855 |
| DDDD | 71 | GLN | 16.6353 | 6.3493 | 24.8641 |
| DDDD | 72 | GLN | 18.9785 | 6.9612 | 28.5924 |
| DDDD | 73 | VAL | 5.8822 | 1.9838 | 11.0801 |
| DDDD | 74 | ASN | 7.4325 | 0.9456 | 13.9195 |
| DDDD | 75 | GLU | 9.6342 | 4.3175 | 13.8875 |
| DDDD | 76 | SER | 0.6597 | 0.9801 | 0.0188 |
| DDDD | 77 | GLU | 15.8994 | 1.2593 | 27.6115 |
| DDDD | 78 | PRO | 8.2500 | 4.9080 | 12.7060 |
| DDDD | 79 | VAL | 6.5718 | 1.5859 | 13.2197 |
| DDDD | 80 | TYR | 7.3618 | 1.8414 | 10.1220 |
| DDDD | 81 | LEU | 0.3727 | 0.0000 | 0.7455 |
| DDDD | 82 | GLU | 5.7273 | 0.0000 | 10.3091 |
| DDDD | 83 | VAL | 1.6083 | 2.8146 | 0.0000 |
| DDDD | 84 | PHE | 2.9246 | 0.6110 | 4.2466 |
| DDDD | 85 | SER | 11.0966 | 6.0084 | 21.2730 |
| DDDD | 86 | ASP | 6.2089 | 2.2133 | 10.2046 |
| DDDD | 87 | TRP | 6.4962 | 0.2557 | 8.9923 |
| DDDD | 88 | LEU | 0.0479 | 0.0958 | 0.0000 |
| DDDD | 89 | LEU | 0.1157 | 0.0000 | 0.2313 |
| DDDD | 90 | LEU | 0.0002 | 0.0000 | 0.0004 |
| DDDD | 91 | GLN | 0.2632 | 0.0000 | 0.4737 |
| DDDD | 92 | ALA | 0.0436 | 0.0427 | 0.0472 |
| DDDD | 93 | SER | 4.5089 | 2.9688 | 7.5891 |
| DDDD | 94 | ALA | 8.3925 | 1.3517 | 36.5557 |
| DDDD | 95 | GLU | 7.1932 | 1.4693 | 11.7724 |
| DDDD | 96 | VAL | 13.5421 | 4.7333 | 25.2870 |
| DDDD | 97 | VAL | 1.5725 | 0.9243 | 2.4367 |
| DDDD | 98 | MET | 14.2776 | 0.0883 | 28.4668 |
| DDDD | 99 | GLU | 7.6854 | 4.8779 | 9.9313 |
| DDDD | 100 | GLY | 4.9621 | 4.9621 | 0.0000 |
| DDDD | 101 | GLN | 9.7282 | 0.0319 | 17.4852 |
| DDDD | 102 | PRO | 9.4269 | 1.5462 | 19.9345 |
| DDDD | 103 | LEU | 0.0306 | 0.0330 | 0.0282 |
| DDDD | 104 | PHE | 6.2490 | 0.0000 | 9.8199 |
| DDDD | 105 | LEU | 0.0962 | 0.0653 | 0.1272 |
| DDDD | 106 | ARG | 2.2407 | 0.0007 | 3.5207 |
| DDDD | 107 | CYS | 0.6463 | 0.9694 | 0.0000 |
| DDDD | 108 | HIS | 1.1583 | 0.2394 | 1.7709 |
| DDDD | 109 | GLY | 1.3558 | 1.3558 | 0.0000 |
| DDDD | 110 | TRP | 4.6167 | 0.3206 | 6.3351 |
| DDDD | 111 | ARG | 13.1658 | 6.9018 | 16.7453 |
| DDDD | 112 | ASN | 12.9428 | 4.3861 | 21.4995 |
| DDDD | 113 | TRP | 6.0476 | 2.8196 | 7.3388 |
| DDDD | 114 | ASP | 11.3414 | 1.8363 | 20.8466 |
| DDDD | 115 | VAL | 0.8809 | 0.7036 | 1.1174 |
| DDDD | 116 | TYR | 5.3412 | 0.0000 | 8.0118 |
| DDDD | 117 | LYS | 8.0269 | 0.7095 | 13.8809 |
| DDDD | 118 | VAL | 0.0001 | 0.0000 | 0.0002 |
| DDDD | 119 | ILE | 1.6342 | 0.0000 | 3.2684 |
| DDDD | 120 | TYR | 0.0131 | 0.0000 | 0.0197 |
| DDDD | 121 | TYR | 2.9992 | 0.0083 | 4.4947 |
| DDDD | 122 | LYS | 4.0023 | 0.8041 | 6.5609 |
| DDDD | 123 | ASP | 10.7259 | 6.1708 | 15.2810 |
| DDDD | 124 | GLY | 14.3365 | 14.3365 | 0.0000 |
| DDDD | 125 | GLU | 7.3185 | 0.3720 | 12.8756 |
| DDDD | 126 | ALA | 2.1423 | 1.1064 | 6.2860 |
| DDDD | 127 | LEU | 5.9569 | 1.4445 | 10.4694 |
| DDDD | 128 | LYS | 11.1254 | 0.2785 | 19.8028 |
| DDDD | 129 | TYR | 5.7025 | 4.2384 | 6.4346 |
| DDDD | 130 | TRP | 6.8913 | 1.0864 | 9.2132 |
| DDDD | 131 | TYR | 4.0982 | 0.8881 | 5.7033 |
| DDDD | 132 | GLU | 11.0810 | 2.0497 | 18.3061 |
| DDDD | 133 | ASN | 5.7438 | 5.5427 | 5.9449 |
| DDDD | 134 | HIS | 8.0681 | 1.4827 | 12.4583 |
| DDDD | 135 | ASN | 1.3522 | 0.3616 | 2.3429 |
| DDDD | 136 | ILE | 2.1277 | 1.2709 | 2.9846 |
| DDDD | 137 | SER | 10.0928 | 7.5360 | 15.2065 |
| DDDD | 138 | ILE | 3.4325 | 0.8858 | 5.9792 |
| DDDD | 139 | THR | 15.7856 | 2.5286 | 33.4616 |
| DDDD | 140 | ASN | 6.3849 | 3.6475 | 9.1223 |
| DDDD | 141 | ALA | 0.0000 | 0.0000 | 0.0000 |
| DDDD | 142 | THR | 7.3411 | 0.2496 | 16.7963 |
| DDDD | 143 | VAL | 7.7635 | 1.1808 | 16.5403 |
| DDDD | 144 | GLU | 13.9706 | 4.2430 | 21.7527 |
| DDDD | 145 | ASP | 4.2654 | 0.0465 | 8.4843 |
| DDDD | 146 | SER | 5.4380 | 3.9844 | 8.3451 |
| DDDD | 147 | GLY | 2.9908 | 2.9908 | 0.0000 |
| DDDD | 148 | THR | 5.6821 | 0.0926 | 13.1348 |
| DDDD | 149 | TYR | 0.2615 | 0.0000 | 0.3922 |
| DDDD | 150 | TYR | 3.8602 | 0.0001 | 5.7903 |
| DDDD | 151 | CYS | 0.0000 | 0.0000 | 0.0000 |
| DDDD | 152 | THR | 3.8063 | 0.0000 | 8.8814 |
| DDDD | 153 | GLY | 1.1071 | 1.1071 | 0.0000 |
| DDDD | 154 | LYS | 5.9720 | 0.0003 | 10.7494 |
| DDDD | 155 | VAL | 0.4744 | 0.0002 | 1.1066 |
| DDDD | 156 | TRP | 3.1754 | 2.8159 | 3.3193 |
| DDDD | 157 | GLN | 10.0034 | 3.2877 | 15.3759 |
| DDDD | 158 | LEU | 14.1226 | 0.2738 | 27.9715 |
| DDDD | 159 | ASP | 7.9805 | 2.3321 | 13.6289 |
| DDDD | 160 | TYR | 3.5343 | 2.4074 | 4.0978 |
| DDDD | 161 | GLU | 12.6759 | 4.6202 | 19.1204 |
| DDDD | 162 | SER | 1.1111 | 1.6667 | 0.0000 |
| DDDD | 163 | GLU | 9.9628 | 0.6047 | 17.4493 |
| DDDD | 164 | PRO | 10.2553 | 1.5732 | 21.8314 |
| DDDD | 165 | LEU | 1.6609 | 0.1704 | 3.1515 |
| DDDD | 166 | ASN | 4.3037 | 1.0892 | 7.5182 |
| DDDD | 167 | ILE | 0.5355 | 1.0632 | 0.0079 |
| DDDD | 168 | THR | 9.9863 | 0.0926 | 23.1779 |
| DDDD | 169 | VAL | 0.5843 | 0.9875 | 0.0466 |
| DDDD | 170 | ILE | 10.6158 | 1.4450 | 19.7865 |
| DDDD | 171 | LYS | 20.3127 | 15.3369 | 24.2933 |
| DDDD | 221 | NAG | 13.3953 | 0.0000 | 13.3953 |
| DDDD | 222 | NAG | 19.9723 | 0.0000 | 19.9723 |
| DDDD | 242 | NAG | 9.9493 | 0.0000 | 9.9493 |
| DDDD | 243 | NAG | 9.3637 | 0.0000 | 9.3637 |
| DDDD | 244 | MAN | 18.7429 | 0.0000 | 18.7429 |
| DDDD | 250 | NAG | 16.0945 | 0.0000 | 16.0945 |
| DDDD | 274 | NAG | 21.9996 | 0.0000 | 21.9996 |
| DDDD | 335 | NAG | 15.1906 | 0.0000 | 15.1906 |
| DDDD | 340 | NAG | 17.8940 | 0.0000 | 17.8940 |
| DDDD | 366 | NAG | 14.6791 | 0.0000 | 14.6791 |
| DDDD | 367 | NAG | 20.8557 | 0.0000 | 20.8557 |
| EEEE | 4 | LYS | 22.5960 | 10.2165 | 32.4995 |
| EEEE | 5 | PRO | 1.1597 | 1.6130 | 0.5553 |
| EEEE | 6 | LYS | 16.8781 | 1.3580 | 29.2941 |
| EEEE | 7 | VAL | 1.6131 | 2.7766 | 0.0619 |
| EEEE | 8 | SER | 8.2297 | 1.9067 | 20.8757 |
| EEEE | 9 | LEU | 4.4542 | 5.4841 | 3.4242 |
| EEEE | 10 | ASN | 11.9578 | 0.9345 | 22.9812 |
| EEEE | 11 | PRO | 8.0892 | 0.4787 | 18.2367 |
| EEEE | 12 | PRO | 10.2667 | 2.2261 | 20.9875 |
| EEEE | 13 | TRP | 1.5846 | 0.0803 | 2.1863 |
| EEEE | 14 | ASN | 3.3863 | 0.3258 | 6.4468 |
| EEEE | 15 | ARG | 1.7357 | 0.0000 | 2.7275 |
| EEEE | 16 | ILE | 0.9829 | 0.0000 | 1.9657 |
| EEEE | 17 | PHE | 0.3246 | 0.0002 | 0.5100 |
| EEEE | 18 | LYS | 10.8388 | 3.5327 | 16.6837 |
| EEEE | 19 | GLY | 5.3803 | 5.3803 | 0.0000 |
| EEEE | 20 | GLU | 3.5458 | 0.0335 | 6.3557 |
| EEEE | 21 | ASN | 5.3181 | 4.0487 | 6.5875 |
| EEEE | 22 | VAL | 0.2825 | 0.4415 | 0.0706 |
| EEEE | 23 | THR | 5.3581 | 0.0670 | 12.4128 |
| EEEE | 24 | LEU | 0.4756 | 0.0000 | 0.9513 |
| EEEE | 25 | THR | 5.2190 | 0.0000 | 12.1778 |
| EEEE | 26 | CYS | 0.3475 | 0.4090 | 0.2246 |
| EEEE | 27 | ASN | 8.6206 | 1.5643 | 15.6770 |
| EEEE | 28 | GLY | 7.3744 | 7.3744 | 0.0000 |
| EEEE | 29 | ASN | 16.8741 | 8.5078 | 25.2404 |
| EEEE | 30 | ASN | 14.4860 | 9.6381 | 19.3339 |
| EEEE | 31 | PHE | 18.9288 | 9.2491 | 24.4601 |
| EEEE | 32 | PHE | 7.2780 | 6.7628 | 7.5723 |
| EEEE | 33 | GLU | 17.3776 | 4.9213 | 27.3427 |
| EEEE | 34 | VAL | 9.8146 | 3.7193 | 17.9417 |
| EEEE | 35 | SER | 14.4525 | 2.2438 | 38.8698 |
| EEEE | 36 | SER | 7.1292 | 1.8233 | 17.7410 |
| EEEE | 37 | THR | 0.7268 | 1.2719 | 0.0000 |
| EEEE | 38 | LYS | 8.8146 | 0.3416 | 15.5930 |
| EEEE | 39 | TRP | 0.0112 | 0.0389 | 0.0002 |
| EEEE | 40 | PHE | 3.0771 | 0.0000 | 4.8354 |
| EEEE | 41 | HIS | 3.3339 | 0.3893 | 5.2969 |
| EEEE | 42 | ASN | 6.3403 | 7.2548 | 5.4259 |
| EEEE | 43 | GLY | 8.8849 | 8.8849 | 0.0000 |
| EEEE | 44 | SER | 12.3851 | 3.1501 | 30.8551 |
| EEEE | 45 | LEU | 14.5997 | 6.7706 | 22.4287 |
| EEEE | 46 | SER | 6.2344 | 4.5208 | 9.6615 |
| EEEE | 47 | GLU | 19.8124 | 8.1501 | 29.1422 |
| EEEE | 48 | GLU | 5.5829 | 2.2603 | 8.2411 |
| EEEE | 49 | THR | 11.1823 | 0.0000 | 26.0920 |
| EEEE | 50 | ASN | 3.7514 | 1.0379 | 6.4648 |
| EEEE | 51 | SER | 1.1828 | 0.0001 | 3.5482 |
| EEEE | 52 | SER | 2.6847 | 0.0000 | 8.0542 |
| EEEE | 53 | LEU | 2.8988 | 0.0160 | 5.7815 |
| EEEE | 54 | ASN | 7.0295 | 3.8406 | 10.2183 |
| EEEE | 55 | ILE | 1.9774 | 1.5267 | 2.4282 |
| EEEE | 56 | VAL | 11.4385 | 1.7455 | 24.3625 |
| EEEE | 57 | ASN | 8.9737 | 1.1309 | 16.8164 |
| EEEE | 58 | ALA | 0.3534 | 0.4418 | 0.0000 |
| EEEE | 59 | LYS | 14.0513 | 0.0995 | 25.2128 |
| EEEE | 60 | PHE | 3.9435 | 0.0489 | 6.1690 |
| EEEE | 61 | GLU | 9.2441 | 0.1331 | 16.5329 |
| EEEE | 62 | ASP | 4.0153 | 0.0793 | 7.9514 |
| EEEE | 63 | SER | 0.1893 | 0.0000 | 0.5678 |
| EEEE | 64 | GLY | 0.0000 | 0.0000 | 0.0000 |
| EEEE | 65 | GLU | 2.9838 | 0.0460 | 5.3340 |
| EEEE | 66 | TYR | 1.0048 | 0.0006 | 1.5069 |
| EEEE | 67 | LYS | 3.2772 | 0.0001 | 5.8989 |
| EEEE | 68 | CYS | 0.0000 | 0.0000 | 0.0000 |
| EEEE | 69 | GLN | 3.9654 | 0.0000 | 7.1378 |
| EEEE | 70 | HIS | 2.6414 | 0.2451 | 4.2389 |
| EEEE | 71 | GLN | 9.7531 | 6.2755 | 12.5353 |
| EEEE | 72 | GLN | 15.3796 | 6.7847 | 22.2555 |
| EEEE | 73 | VAL | 6.0006 | 2.3479 | 10.8709 |
| EEEE | 74 | ASN | 7.6007 | 0.9858 | 14.2156 |
| EEEE | 75 | GLU | 9.6042 | 4.4474 | 13.7297 |
| EEEE | 76 | SER | 0.6746 | 1.0066 | 0.0106 |
| EEEE | 77 | GLU | 12.7067 | 1.2232 | 21.8935 |
| EEEE | 78 | PRO | 2.3956 | 1.5978 | 3.4593 |
| EEEE | 79 | VAL | 6.0444 | 0.7862 | 13.0553 |
| EEEE | 80 | TYR | 4.1212 | 1.3782 | 5.4928 |
| EEEE | 81 | LEU | 0.4531 | 0.0000 | 0.9061 |
| EEEE | 82 | GLU | 5.5155 | 0.0000 | 9.9279 |
| EEEE | 83 | VAL | 1.5309 | 2.6784 | 0.0008 |
| EEEE | 84 | PHE | 2.8834 | 0.6355 | 4.1679 |
| EEEE | 85 | SER | 11.0911 | 6.1575 | 20.9584 |
| EEEE | 86 | ASP | 6.4143 | 2.5687 | 10.2598 |
| EEEE | 87 | TRP | 9.2857 | 0.2426 | 12.9030 |
| EEEE | 88 | LEU | 0.0519 | 0.1037 | 0.0000 |
| EEEE | 89 | LEU | 0.1205 | 0.0000 | 0.2410 |
| EEEE | 90 | LEU | 0.0000 | 0.0000 | 0.0000 |
| EEEE | 91 | GLN | 0.2686 | 0.0000 | 0.4835 |
| EEEE | 92 | ALA | 0.0943 | 0.0991 | 0.0752 |
| EEEE | 93 | SER | 4.2341 | 2.9403 | 6.8217 |
| EEEE | 94 | ALA | 8.4724 | 1.4590 | 36.5259 |
| EEEE | 95 | GLU | 4.3753 | 1.4778 | 6.6933 |
| EEEE | 96 | VAL | 4.5984 | 4.5523 | 4.6599 |
| EEEE | 97 | VAL | 1.5521 | 0.9731 | 2.3241 |
| EEEE | 98 | MET | 14.3494 | 0.0687 | 28.6301 |
| EEEE | 99 | GLU | 7.4147 | 5.0436 | 9.3116 |
| EEEE | 100 | GLY | 5.3477 | 5.3477 | 0.0000 |
| EEEE | 101 | GLN | 9.6429 | 0.0120 | 17.3476 |
| EEEE | 102 | PRO | 9.4892 | 1.6653 | 19.9211 |
| EEEE | 103 | LEU | 0.0234 | 0.0192 | 0.0275 |
| EEEE | 104 | PHE | 6.1968 | 0.0000 | 9.7379 |
| EEEE | 105 | LEU | 0.0865 | 0.0657 | 0.1072 |
| EEEE | 106 | ARG | 2.2169 | 0.0000 | 3.4838 |
| EEEE | 107 | CYS | 0.6436 | 0.9654 | 0.0000 |
| EEEE | 108 | HIS | 1.1724 | 0.2409 | 1.7935 |
| EEEE | 109 | GLY | 1.2996 | 1.2996 | 0.0000 |
| EEEE | 110 | TRP | 4.6388 | 0.3429 | 6.3572 |
| EEEE | 111 | ARG | 14.4865 | 6.9035 | 18.8197 |
| EEEE | 112 | ASN | 13.2482 | 4.3960 | 22.1003 |
| EEEE | 113 | TRP | 7.0238 | 3.3035 | 8.5119 |
| EEEE | 114 | ASP | 11.8883 | 1.9586 | 21.8181 |
| EEEE | 115 | VAL | 0.8842 | 0.7257 | 1.0956 |
| EEEE | 116 | TYR | 6.0204 | 0.0000 | 9.0306 |
| EEEE | 117 | LYS | 11.3486 | 0.7247 | 19.8476 |
| EEEE | 118 | VAL | 0.0000 | 0.0000 | 0.0000 |
| EEEE | 119 | ILE | 4.3850 | 0.0000 | 8.7700 |
| EEEE | 120 | TYR | 0.0002 | 0.0005 | 0.0000 |
| EEEE | 121 | TYR | 3.8442 | 0.0044 | 5.7642 |
| EEEE | 122 | LYS | 4.0492 | 0.9747 | 6.5088 |
| EEEE | 123 | ASP | 10.6363 | 6.3573 | 14.9153 |
| EEEE | 124 | GLY | 14.2853 | 14.2853 | 0.0000 |
| EEEE | 125 | GLU | 13.3618 | 0.4051 | 23.7272 |
| EEEE | 126 | ALA | 14.6809 | 5.6541 | 50.7878 |
| EEEE | 127 | LEU | 9.2613 | 6.4361 | 12.0866 |
| EEEE | 128 | LYS | 11.7127 | 1.6189 | 19.7878 |
| EEEE | 129 | TYR | 10.5042 | 5.1382 | 13.1872 |
| EEEE | 130 | TRP | 8.3076 | 1.0723 | 11.2017 |
| EEEE | 131 | TYR | 12.1072 | 0.8991 | 17.7113 |
| EEEE | 132 | GLU | 12.7199 | 2.0028 | 21.2936 |
| EEEE | 133 | ASN | 5.6925 | 5.5621 | 5.8228 |
| EEEE | 134 | HIS | 8.1921 | 1.5201 | 12.6401 |
| EEEE | 135 | ASN | 1.3201 | 0.2942 | 2.3461 |
| EEEE | 136 | ILE | 2.2145 | 1.3058 | 3.1231 |
| EEEE | 137 | SER | 10.0571 | 7.3406 | 15.4902 |
| EEEE | 138 | ILE | 3.4381 | 0.9086 | 5.9677 |
| EEEE | 139 | THR | 15.7625 | 2.5279 | 33.4087 |
| EEEE | 140 | ASN | 6.4209 | 3.6811 | 9.1607 |
| EEEE | 141 | ALA | 0.0000 | 0.0000 | 0.0000 |
| EEEE | 142 | THR | 7.2538 | 0.3369 | 16.4763 |
| EEEE | 143 | VAL | 5.2826 | 1.1302 | 10.8192 |
| EEEE | 144 | GLU | 14.2599 | 4.2746 | 22.2482 |
| EEEE | 145 | ASP | 4.3200 | 0.0534 | 8.5866 |
| EEEE | 146 | SER | 5.5098 | 4.0762 | 8.3771 |
| EEEE | 147 | GLY | 2.9433 | 2.9433 | 0.0000 |
| EEEE | 148 | THR | 5.7039 | 0.0995 | 13.1764 |
| EEEE | 149 | TYR | 0.2552 | 0.0130 | 0.3763 |
| EEEE | 150 | TYR | 3.8275 | 0.0000 | 5.7413 |
| EEEE | 151 | CYS | 0.0000 | 0.0000 | 0.0000 |
| EEEE | 152 | THR | 3.7660 | 0.0000 | 8.7874 |
| EEEE | 153 | GLY | 1.1095 | 1.1095 | 0.0000 |
| EEEE | 154 | LYS | 6.0705 | 0.0037 | 10.9239 |
| EEEE | 155 | VAL | 0.4853 | 0.0000 | 1.1323 |
| EEEE | 156 | TRP | 11.8745 | 5.3337 | 14.4908 |
| EEEE | 157 | GLN | 14.3320 | 3.3004 | 23.1573 |
| EEEE | 158 | LEU | 13.6525 | 0.2539 | 27.0512 |
| EEEE | 159 | ASP | 14.3336 | 5.0741 | 23.5931 |
| EEEE | 160 | TYR | 3.5095 | 2.3905 | 4.0689 |
| EEEE | 161 | GLU | 13.4677 | 5.2742 | 20.0225 |
| EEEE | 162 | SER | 1.1284 | 1.6927 | 0.0000 |
| EEEE | 163 | GLU | 9.6823 | 0.5318 | 17.0027 |
| EEEE | 164 | PRO | 10.3139 | 1.5274 | 22.0292 |
| EEEE | 165 | LEU | 1.6379 | 0.1485 | 3.1273 |
| EEEE | 166 | ASN | 3.3639 | 0.7774 | 5.9503 |
| EEEE | 167 | ILE | 0.5534 | 1.0911 | 0.0157 |
| EEEE | 168 | THR | 3.6331 | 0.0674 | 8.3873 |
| EEEE | 169 | VAL | 0.0817 | 0.1078 | 0.0468 |
| EEEE | 170 | ILE | 2.1648 | 0.0777 | 4.2519 |
| EEEE | 171 | LYS | 14.9019 | 13.4622 | 16.0537 |
| EEEE | 221 | NAG | 13.0723 | 0.0000 | 13.0723 |
| EEEE | 222 | NAG | 20.3453 | 0.0000 | 20.3453 |
| EEEE | 242 | NAG | 8.8452 | 0.0000 | 8.8452 |
| EEEE | 243 | NAG | 7.6625 | 0.0000 | 7.6625 |
| EEEE | 244 | MAN | 18.6073 | 0.0000 | 18.6073 |
| EEEE | 250 | NAG | 16.1217 | 0.0000 | 16.1217 |
| EEEE | 274 | NAG | 22.0349 | 0.0000 | 22.0349 |
| EEEE | 335 | NAG | 15.0552 | 0.0000 | 15.0552 |
| EEEE | 340 | NAG | 17.7238 | 0.0000 | 17.7238 |
| EEEE | 366 | NAG | 12.1825 | 0.0000 | 12.1825 |
| EEEE | 367 | NAG | 19.5820 | 0.0000 | 19.5820 |

TABLE 11
PhFcεRIα₁₋₁₇₆, Form M2, residue exposure
>>>> coordinate set = md6c1.pdb

| segid | resid | resname | access | access-main | access-side |
|---|---|---|---|---|---|
| AAAA | 1 | VAL | 23.3378 | 10.2131 | 40.8375 |
| AAAA | 2 | PRO | 11.8969 | 4.7510 | 21.4247 |
| AAAA | 3 | GLN | 4.0040 | 0.6188 | 6.7120 |
| AAAA | 4 | LYS | 10.6487 | 3.0641 | 16.7164 |
| AAAA | 5 | PRO | 0.2700 | 0.1467 | 0.4343 |
| AAAA | 6 | LYS | 14.5164 | 0.1128 | 26.0393 |
| AAAA | 7 | VAL | 2.0175 | 3.5306 | 0.0002 |
| AAAA | 8 | SER | 8.4156 | 1.5950 | 22.0570 |
| AAAA | 9 | LEU | 3.4044 | 3.6981 | 3.1106 |
| AAAA | 10 | ASN | 11.5698 | 0.5893 | 22.5503 |
| AAAA | 11 | PRO | 8.5175 | 0.1465 | 19.6787 |
| AAAA | 12 | PRO | 9.1363 | 1.5259 | 19.2835 |
| AAAA | 13 | TRP | 2.0981 | 0.0033 | 2.9360 |
| AAAA | 14 | ASN | 2.7536 | 0.0000 | 5.5073 |
| AAAA | 15 | ARG | 0.7887 | 0.0000 | 1.2394 |
| AAAA | 16 | ILE | 0.5825 | 0.0000 | 1.1649 |
| AAAA | 17 | PHE | 0.1853 | 0.0000 | 0.2912 |
| AAAA | 18 | LYS | 9.6106 | 1.2098 | 16.3312 |
| AAAA | 19 | GLY | 4.3200 | 4.3200 | 0.0000 |
| AAAA | 20 | GLU | 2.6272 | 0.0000 | 4.7290 |
| AAAA | 21 | ASN | 4.7245 | 2.9284 | 6.5206 |
| AAAA | 22 | VAL | 0.4741 | 0.6955 | 0.1788 |
| AAAA | 23 | THR | 4.7669 | 0.0001 | 11.1226 |
| AAAA | 24 | LEU | 0.0001 | 0.0000 | 0.0002 |
| AAAA | 25 | THR | 5.8774 | 0.0006 | 13.7131 |
| AAAA | 26 | CYS | 1.2474 | 1.8711 | 0.0000 |
| AAAA | 27 | ASN | 9.8972 | 1.4492 | 18.3453 |
| AAAA | 28 | GLY | 11.8125 | 11.8125 | 0.0000 |
| AAAA | 29 | ASN | 10.4976 | 5.7985 | 15.1967 |
| AAAA | 30 | ASN | 17.1596 | 4.7289 | 29.5903 |
| AAAA | 31 | PHE | 14.9024 | 9.0487 | 18.2474 |
| AAAA | 32 | PHE | 6.9262 | 1.1011 | 10.2548 |
| AAAA | 33 | GLU | 19.3638 | 7.9005 | 28.5344 |
| AAAA | 34 | VAL | 10.5040 | 9.2873 | 12.1262 |
| AAAA | 35 | SER | 20.0797 | 11.1239 | 37.9912 |
| AAAA | 36 | SER | 10.3115 | 2.5541 | 25.8262 |
| AAAA | 37 | THR | 0.3123 | 0.1509 | 0.5275 |
| AAAA | 38 | LYS | 9.1055 | 0.0303 | 16.3656 |
| AAAA | 39 | TRP | 0.0125 | 0.0004 | 0.0173 |
| AAAA | 40 | PHE | 3.3329 | 0.0507 | 5.2085 |
| AAAA | 41 | HIS | 3.3604 | 0.4369 | 5.3093 |
| AAAA | 42 | ASN | 5.7196 | 5.9748 | 5.4644 |
| AAAA | 43 | GLY | 11.0441 | 11.0441 | 0.0000 |
| AAAA | 44 | SER | 12.3468 | 1.6054 | 33.8295 |
| AAAA | 45 | LEU | 12.4194 | 6.6727 | 18.1661 |
| AAAA | 46 | SER | 6.2970 | 3.5164 | 11.8583 |
| AAAA | 47 | GLU | 19.2754 | 6.3506 | 29.6153 |
| AAAA | 48 | GLU | 5.5497 | 2.2183 | 8.2149 |
| AAAA | 49 | THR | 10.8597 | 1.3240 | 23.5740 |
| AAAA | 50 | ASN | 12.0836 | 1.0849 | 23.0822 |
| AAAA | 51 | SER | 8.5667 | 0.8380 | 24.0243 |
| AAAA | 52 | SER | 6.5795 | 0.5607 | 18.6170 |
| AAAA | 53 | LEU | 2.0088 | 0.0000 | 4.0175 |
| AAAA | 54 | ASN | 10.4631 | 4.8106 | 16.1155 |
| AAAA | 55 | ILE | 1.5373 | 1.3922 | 1.6825 |
| AAAA | 56 | VAL | 9.8664 | 4.5674 | 16.9318 |
| AAAA | 57 | ASN | 8.3728 | 1.0485 | 15.6970 |
| AAAA | 58 | ALA | 0.1673 | 0.2091 | 0.0000 |
| AAAA | 59 | LYS | 12.7698 | 0.0135 | 22.9748 |
| AAAA | 60 | PHE | 2.9685 | 0.0000 | 4.6647 |
| AAAA | 61 | GLU | 10.1438 | 0.3798 | 17.9550 |
| AAAA | 62 | ASP | 3.8187 | 0.0000 | 7.6375 |
| AAAA | 63 | SER | 0.0423 | 0.0002 | 0.1265 |
| AAAA | 64 | GLY | 0.7550 | 0.7550 | 0.0000 |
| AAAA | 65 | GLU | 4.0298 | 0.0243 | 7.2343 |
| AAAA | 66 | TYR | 0.5144 | 0.0000 | 0.7716 |
| AAAA | 67 | LYS | 4.6070 | 0.0021 | 8.2910 |
| AAAA | 68 | CYS | 0.0643 | 0.0965 | 0.0000 |
| AAAA | 69 | GLN | 4.3129 | 0.5930 | 7.2889 |
| AAAA | 70 | HIS | 2.2107 | 1.4131 | 2.7425 |
| AAAA | 71 | GLN | 15.8607 | 4.7123 | 24.7794 |
| AAAA | 72 | GLN | 10.1949 | 4.9757 | 14.3702 |
| AAAA | 73 | VAL | 4.5886 | 3.4088 | 6.1618 |
| AAAA | 74 | ASN | 7.2228 | 1.6554 | 12.7903 |
| AAAA | 75 | GLU | 11.1970 | 3.1686 | 17.6196 |
| AAAA | 76 | SER | 0.7529 | 1.1293 | 0.0000 |
| AAAA | 77 | GLU | 5.6624 | 0.5155 | 9.7799 |
| AAAA | 78 | PRO | 9.4668 | 4.0586 | 16.6776 |
| AAAA | 79 | VAL | 4.2206 | 0.7903 | 8.7943 |
| AAAA | 80 | TYR | 10.8696 | 1.6448 | 15.4820 |
| AAAA | 81 | LEU | 0.3295 | 0.6590 | 0.0000 |
| AAAA | 82 | GLU | 6.5599 | 0.0000 | 11.8078 |
| AAAA | 83 | VAL | 1.2313 | 2.1548 | 0.0000 |
| AAAA | 84 | PHE | 2.6783 | 1.0628 | 3.6015 |
| AAAA | 85 | SER | 10.4042 | 7.2453 | 16.7222 |
| AAAA | 86 | ASP | 6.7155 | 3.1365 | 10.2944 |
| AAAA | 87 | TRP | 7.9670 | 0.0000 | 11.1538 |
| AAAA | 88 | LEU | 0.2303 | 0.4605 | 0.0000 |
| AAAA | 89 | LEU | 0.1824 | 0.0005 | 0.3643 |
| AAAA | 90 | LEU | 0.0000 | 0.0000 | 0.0000 |
| AAAA | 91 | GLN | 0.1542 | 0.0000 | 0.2776 |
| AAAA | 92 | ALA | 0.0000 | 0.0000 | 0.0000 |
| AAAA | 93 | SER | 6.4731 | 4.6474 | 10.1245 |
| AAAA | 94 | ALA | 6.9800 | 1.7371 | 27.9520 |
| AAAA | 95 | GLU | 6.3625 | 0.8834 | 10.7457 |
| AAAA | 96 | VAL | 12.7032 | 5.5496 | 22.2413 |
| AAAA | 97 | VAL | 1.4127 | 0.9895 | 1.9769 |
| AAAA | 98 | MET | 8.3663 | 0.9908 | 15.7417 |
| AAAA | 99 | GLU | 6.0466 | 3.8025 | 7.8419 |
| AAAA | 100 | GLY | 1.3823 | 1.3823 | 0.0000 |
| AAAA | 101 | GLN | 9.3401 | 0.0000 | 16.8122 |
| AAAA | 102 | PRO | 11.5211 | 1.3350 | 25.1025 |
| AAAA | 103 | LEU | 0.2239 | 0.2968 | 0.1510 |
| AAAA | 104 | PHE | 5.5960 | 0.0000 | 8.7937 |
| AAAA | 105 | LEU | 0.2800 | 0.0000 | 0.5599 |
| AAAA | 106 | ARG | 5.6019 | 0.0000 | 8.8030 |
| AAAA | 107 | CYS | 1.9041 | 2.6369 | 0.4387 |
| AAAA | 108 | HIS | 1.2459 | 0.8447 | 1.5133 |
| AAAA | 109 | GLY | 0.2958 | 0.2958 | 0.0000 |
| AAAA | 110 | TRP | 3.6776 | 0.2682 | 5.0414 |
| AAAA | 111 | ARG | 13.9748 | 6.0118 | 18.5251 |
| AAAA | 112 | ASN | 13.0426 | 5.9312 | 20.1540 |
| AAAA | 113 | TRP | 8.4374 | 2.2626 | 10.9073 |
| AAAA | 114 | ASP | 10.8862 | 0.8382 | 20.9341 |
| AAAA | 115 | VAL | 3.6736 | 4.0353 | 3.1913 |
| AAAA | 116 | TYR | 10.8526 | 0.9359 | 15.8110 |
| AAAA | 117 | LYS | 12.5729 | 4.0303 | 19.4070 |
| AAAA | 118 | VAL | 1.5367 | 1.4181 | 1.6949 |
| AAAA | 119 | ILE | 3.8886 | 0.4794 | 7.2979 |
| AAAA | 120 | TYR | 0.1235 | 0.0400 | 0.1653 |
| AAAA | 121 | TYR | 3.2159 | 0.0000 | 4.8238 |
| AAAA | 122 | LYS | 4.1348 | 0.5235 | 7.0239 |
| AAAA | 123 | ASP | 9.0341 | 4.7242 | 13.3440 |
| AAAA | 124 | GLY | 12.8886 | 12.8886 | 0.0000 |
| AAAA | 125 | GLU | 13.5568 | 0.5821 | 23.9366 |
| AAAA | 126 | ALA | 5.4448 | 3.6508 | 12.6207 |
| AAAA | 127 | LEU | 4.4392 | 1.2148 | 7.6637 |
| AAAA | 128 | LYS | 6.6847 | 0.4089 | 11.7054 |
| AAAA | 129 | TYR | 17.7661 | 6.5522 | 23.3730 |
| AAAA | 130 | TRP | 4.6781 | 0.2956 | 6.4311 |
| AAAA | 131 | TYR | 5.9070 | 2.3743 | 7.6733 |
| AAAA | 132 | GLU | 14.4146 | 6.3584 | 20.8595 |
| AAAA | 133 | ASN | 9.2636 | 0.7631 | 17.7642 |
| AAAA | 134 | HIS | 14.3143 | 1.4912 | 22.8630 |
| AAAA | 135 | ASN | 6.6861 | 0.2683 | 13.1040 |
| AAAA | 136 | ILE | 0.1409 | 0.0100 | 0.2719 |
| AAAA | 137 | SER | 9.4451 | 3.2255 | 21.8842 |
| AAAA | 138 | ILE | 2.6491 | 0.8829 | 4.4154 |
| AAAA | 139 | THR | 12.8859 | 1.4417 | 28.1448 |
| AAAA | 140 | ASN | 6.4432 | 3.5706 | 9.3157 |
| AAAA | 141 | ALA | 1.3406 | 1.6757 | 0.0000 |
| AAAA | 142 | THR | 7.2752 | 0.0020 | 16.9727 |
| AAAA | 143 | VAL | 11.7608 | 1.2461 | 25.7803 |
| AAAA | 144 | GLU | 14.7507 | 2.5626 | 24.5012 |
| AAAA | 145 | ASP | 3.5866 | 0.0546 | 7.1186 |
| AAAA | 146 | SER | 4.2659 | 2.0709 | 8.6561 |
| AAAA | 147 | GLY | 2.4870 | 2.4870 | 0.0000 |
| AAAA | 148 | THR | 3.9797 | 0.0000 | 9.2860 |
| AAAA | 149 | TYR | 0.3266 | 0.0000 | 0.4899 |
| AAAA | 150 | TYR | 3.1527 | 0.0005 | 4.7289 |
| AAAA | 151 | CYS | 0.0003 | 0.0005 | 0.0000 |
| AAAA | 152 | THR | 4.1824 | 0.6979 | 8.8283 |
| AAAA | 153 | GLY | 0.7331 | 0.7331 | 0.0000 |
| AAAA | 154 | LYS | 7.5163 | 0.0133 | 13.5188 |
| AAAA | 155 | VAL | 0.2906 | 0.0000 | 0.6781 |
| AAAA | 156 | TRP | 11.8912 | 1.7474 | 15.9487 |
| AAAA | 157 | GLN | 14.6241 | 5.4565 | 21.9582 |
| AAAA | 158 | LEU | 13.5291 | 0.9340 | 26.1242 |
| AAAA | 159 | ASP | 14.3755 | 5.4004 | 23.3507 |
| AAAA | 160 | TYR | 3.1668 | 2.6149 | 3.4428 |
| AAAA | 161 | GLU | 10.8144 | 4.2630 | 16.0556 |
| AAAA | 162 | SER | 0.5614 | 0.8334 | 0.0172 |
| AAAA | 163 | GLU | 10.6063 | 0.2900 | 18.8593 |
| AAAA | 164 | PRO | 10.9414 | 3.3661 | 21.0417 |
| AAAA | 165 | LEU | 1.9287 | 0.7267 | 3.1308 |
| AAAA | 166 | ASN | 5.8142 | 3.0970 | 8.5314 |
| AAAA | 167 | ILE | 0.2918 | 0.5835 | 0.0000 |
| AAAA | 168 | THR | 9.3327 | 0.0000 | 21.7764 |
| AAAA | 169 | VAL | 0.2835 | 0.4961 | 0.0000 |
| AAAA | 170 | ILE | 10.1702 | 0.5659 | 19.7745 |
| AAAA | 171 | LYS | 14.8660 | 3.9580 | 23.5925 |
| AAAA | 172 | ALA | 10.1636 | 3.7167 | 35.9516 |
| AAAA | 173 | PRO | 16.8141 | 8.4692 | 27.9405 |
| AAAA | 174 | ARG | 24.7335 | 21.3158 | 26.6865 |
| AAAA | 221 | NAG | 10.3017 | 0.0000 | 10.3017 |
| AAAA | 222 | NAG | 20.4990 | 0.0000 | 20.4990 |
| AAAA | 242 | NAG | 10.4998 | 0.0000 | 10.4998 |
| AAAA | 243 | NAG | 9.1915 | 0.0000 | 9.1915 |
| AAAA | 244 | MAN | 17.0951 | 0.0000 | 17.0951 |
| AAAA | 274 | NAG | 8.2536 | 0.0000 | 8.2536 |
| AAAA | 275 | FCA | 13.5116 | 0.0000 | 13.5116 |
| AAAA | 276 | NAG | 18.0492 | 0.0000 | 18.0492 |
| AAAA | 340 | NAG | 18.2117 | 0.0000 | 18.2117 |
| AAAA | 366 | NAG | 20.2201 | 0.0000 | 20.2201 |
| BBBB | 1 | VAL | 23.2202 | 13.0224 | 36.8172 |
| BBBB | 2 | PRO | 12.9287 | 6.5376 | 21.4502 |
| BBBB | 3 | GLN | 7.8969 | 1.1767 | 13.2731 |
| BBBB | 4 | LYS | 10.9639 | 2.4069 | 17.8095 |
| BBBB | 5 | PRO | 0.1485 | 0.1774 | 0.1101 |
| BBBB | 6 | LYS | 13.6900 | 0.3136 | 24.3911 |
| BBBB | 7 | VAL | 2.0335 | 3.5586 | 0.0000 |
| BBBB | 8 | SER | 8.6515 | 1.6168 | 22.7209 |
| BBBB | 9 | LEU | 3.3843 | 3.6519 | 3.1168 |
| BBBB | 10 | ASN | 11.2166 | 0.5892 | 21.8441 |
| BBBB | 11 | PRO | 6.4967 | 0.0774 | 15.0558 |
| BBBB | 12 | PRO | 5.5258 | 1.4792 | 10.9213 |
| BBBB | 13 | TRP | 0.5399 | 0.0000 | 0.7559 |
| BBBB | 14 | ASN | 2.8551 | 0.0000 | 5.7102 |
| BBBB | 15 | ARG | 0.8228 | 0.0000 | 1.2930 |
| BBBB | 16 | ILE | 0.7004 | 0.0000 | 1.4007 |
| BBBB | 17 | PHE | 0.2062 | 0.0000 | 0.3240 |
| BBBB | 18 | LYS | 10.1266 | 2.4178 | 16.2937 |
| BBBB | 19 | GLY | 5.1193 | 5.1193 | 0.0000 |
| BBBB | 20 | GLU | 3.6592 | 0.0000 | 6.5866 |
| BBBB | 21 | ASN | 4.9980 | 2.8696 | 7.1265 |
| BBBB | 22 | VAL | 0.3086 | 0.5358 | 0.0056 |
| BBBB | 23 | THR | 4.8914 | 0.0060 | 11.4053 |
| BBBB | 24 | LEU | 0.0000 | 0.0000 | 0.0000 |
| BBBB | 25 | THR | 5.3161 | 0.0000 | 12.4043 |
| BBBB | 26 | CYS | 1.7698 | 1.9470 | 1.4152 |
| BBBB | 27 | ASN | 9.5595 | 2.4594 | 16.6596 |
| BBBB | 28 | GLY | 5.1022 | 5.1022 | 0.0000 |
| BBBB | 29 | ASN | 11.6239 | 9.1902 | 14.0577 |
| BBBB | 30 | ASN | 11.1354 | 7.5265 | 14.7442 |
| BBBB | 31 | PHE | 12.4823 | 0.9411 | 19.0773 |
| BBBB | 32 | PHE | 14.9629 | 4.4534 | 20.9683 |
| BBBB | 33 | GLU | 10.0579 | 1.1430 | 17.1898 |
| BBBB | 34 | VAL | 8.1169 | 2.1335 | 16.0948 |
| BBBB | 35 | SER | 17.2091 | 9.6972 | 32.2329 |
| BBBB | 36 | SER | 5.6660 | 1.8677 | 13.2627 |
| BBBB | 37 | THR | 0.9190 | 0.7693 | 1.1187 |
| BBBB | 38 | LYS | 10.5493 | 0.0585 | 18.9418 |
| BBBB | 39 | TRP | 0.0160 | 0.0000 | 0.0224 |
| BBBB | 40 | PHE | 3.2085 | 0.1128 | 4.9774 |
| BBBB | 41 | HIS | 3.2674 | 0.7993 | 4.9129 |
| BBBB | 42 | ASN | 6.9206 | 7.0588 | 6.7824 |
| BBBB | 43 | GLY | 10.5521 | 10.5521 | 0.0000 |
| BBBB | 44 | SER | 12.5873 | 1.8007 | 34.1606 |
| BBBB | 45 | LEU | 12.5684 | 7.0671 | 18.0697 |
| BBBB | 46 | SER | 5.8736 | 2.9025 | 11.8159 |
| BBBB | 47 | GLU | 18.2898 | 4.6076 | 29.2356 |
| BBBB | 48 | GLU | 6.4732 | 2.2413 | 9.8587 |
| BBBB | 49 | THR | 12.4950 | 1.2186 | 27.5302 |
| BBBB | 50 | ASN | 10.6353 | 1.9696 | 19.3010 |
| BBBB | 51 | SER | 2.7922 | 0.0131 | 8.3506 |
| BBBB | 52 | SER | 5.4540 | 0.4267 | 15.5085 |
| BBBB | 53 | LEU | 2.2138 | 0.0004 | 4.4271 |
| BBBB | 54 | ASN | 10.5005 | 4.6511 | 16.3499 |
| BBBB | 55 | ILE | 1.3385 | 1.1102 | 1.5667 |
| BBBB | 56 | VAL | 11.5067 | 3.9707 | 21.5548 |
| BBBB | 57 | ASN | 8.7141 | 1.1097 | 16.3185 |
| BBBB | 58 | ALA | 0.1840 | 0.2300 | 0.0000 |
| BBBB | 59 | LYS | 13.0236 | 0.0000 | 23.4424 |
| BBBB | 60 | PHE | 2.3308 | 0.0000 | 3.6627 |
| BBBB | 61 | GLU | 9.4233 | 0.1906 | 16.8095 |
| BBBB | 62 | ASP | 3.8339 | 0.0000 | 7.6678 |
| BBBB | 63 | SER | 0.1605 | 0.0000 | 0.4815 |
| BBBB | 64 | GLY | 1.6421 | 1.6421 | 0.0000 |
| BBBB | 65 | GLU | 3.9224 | 0.0377 | 7.0302 |
| BBBB | 66 | TYR | 0.5105 | 0.0000 | 0.7658 |
| BBBB | 67 | LYS | 3.9749 | 0.0002 | 7.1547 |
| BBBB | 68 | CYS | 0.0929 | 0.1393 | 0.0000 |
| BBBB | 69 | GLN | 5.4367 | 0.1443 | 9.6707 |
| BBBB | 70 | HIS | 4.9806 | 0.9868 | 7.6431 |
| BBBB | 71 | GLN | 14.5333 | 5.4826 | 21.7740 |
| BBBB | 72 | GLN | 18.4063 | 11.6333 | 23.8247 |
| BBBB | 73 | VAL | 2.6548 | 3.2121 | 1.9118 |
| BBBB | 74 | ASN | 12.0029 | 2.0287 | 21.9770 |
| BBBB | 75 | GLU | 8.4921 | 2.6890 | 13.1345 |
| BBBB | 76 | SER | 0.7254 | 1.0881 | 0.0000 |
| BBBB | 77 | GLU | 7.7802 | 0.6132 | 13.5138 |
| BBBB | 78 | PRO | 9.3860 | 3.7419 | 16.9114 |
| BBBB | 79 | VAL | 4.0363 | 0.8626 | 8.2679 |
| BBBB | 80 | TYR | 11.1782 | 1.6916 | 15.9215 |
| BBBB | 81 | LEU | 0.2983 | 0.5965 | 0.0000 |
| BBBB | 82 | GLU | 7.4968 | 0.0000 | 13.4942 |
| BBBB | 83 | VAL | 1.2876 | 2.2532 | 0.0000 |
| BBBB | 84 | PHE | 2.7723 | 1.0285 | 3.7688 |
| BBBB | 85 | SER | 10.1939 | 7.3007 | 15.9804 |
| BBBB | 86 | ASP | 6.5079 | 3.0391 | 9.9768 |
| BBBB | 87 | TRP | 6.1336 | 0.0000 | 8.5870 |
| BBBB | 88 | LEU | 0.2766 | 0.5478 | 0.0054 |
| BBBB | 89 | LEU | 0.2222 | 0.0049 | 0.4394 |
| BBBB | 90 | LEU | 0.0152 | 0.0302 | 0.0001 |
| BBBB | 91 | GLN | 0.1468 | 0.0000 | 0.2642 |
| BBBB | 92 | ALA | 0.0005 | 0.0006 | 0.0000 |
| BBBB | 93 | SER | 5.6156 | 4.5602 | 7.7265 |
| BBBB | 94 | ALA | 6.8297 | 1.8546 | 26.7302 |
| BBBB | 95 | GLU | 6.8738 | 0.8762 | 11.6719 |
| BBBB | 96 | VAL | 12.2316 | 5.3212 | 21.4456 |
| BBBB | 97 | VAL | 1.4488 | 1.1013 | 1.9123 |
| BBBB | 98 | MET | 11.2447 | 0.4365 | 22.0530 |
| BBBB | 99 | GLU | 6.9392 | 5.4744 | 8.1110 |
| BBBB | 100 | GLY | 2.1371 | 2.1371 | 0.0000 |
| BBBB | 101 | GLN | 10.3142 | 0.0031 | 18.5631 |
| BBBB | 102 | PRO | 10.9007 | 1.3692 | 23.6094 |
| BBBB | 103 | LEU | 0.1806 | 0.2907 | 0.0705 |
| BBBB | 104 | PHE | 0.9676 | 0.0002 | 1.5204 |
| BBBB | 105 | LEU | 0.2088 | 0.0016 | 0.4161 |
| BBBB | 106 | ARG | 3.6986 | 0.0002 | 5.8119 |
| BBBB | 107 | CYS | 0.0292 | 0.0438 | 0.0000 |
| BBBB | 108 | HIS | 1.4195 | 0.6184 | 1;9535 |
| BBBB | 109 | GLY | 0.5887 | 0.5887 | 0.0000 |
| BBBB | 110 | TRP | 3.8590 | 0.0000 | 5.4025 |
| BBBB | 111 | ARG | 12.1336 | 6.9873 | 15.0744 |
| BBBB | 112 | ASN | 13.9325 | 3.3709 | 24.4942 |
| BBBB | 113 | TRP | 3.3478 | 2.2053 | 3.8048 |
| BBBB | 114 | ASP | 7.6950 | 3.0188 | 12.3711 |
| BBBB | 115 | VAL | 0.1840 | 0.2489 | 0.0975 |
| BBBB | 116 | TYR | 4.9222 | 0.0000 | 7.3832 |
| BBBB | 117 | LYS | 10.4451 | 0.8887 | 18.0902 |
| BBBB | 118 | VAL | 0.0000 | 0.0000 | 0.0000 |
| BBBB | 119 | ILE | 4.6639 | 0.0004 | 9.3274 |
| BBBB | 120 | TYR | 0.0002 | 0.0000 | 0.0003 |
| BBBB | 121 | TYR | 3.8872 | 0.0000 | 5.8308 |
| BBBB | 122 | LYS | 4.3212 | 0.6213 | 7.2812 |
| BBBB | 123 | ASP | 9.3317 | 5.1768 | 13.4866 |
| BBBB | 124 | GLY | 14.0149 | 14.0149 | 0.0000 |
| BBBB | 125 | GLU | 14.1323 | 1.6253 | 24.1378 |
| BBBB | 126 | ALA | 12.2018 | 6.3993 | 35.4121 |
| BBBB | 127 | LEU | 9.7819 | 5.9893 | 13.5746 |
| BBBB | 128 | LYS | 10.6006 | 2.2370 | 17.2915 |
| BBBB | 129 | TYR | 9.1175 | 4.6166 | 11.3680 |
| BBBB | 130 | TRP | 11.8627 | 0.8908 | 16.2515 |
| BBBB | 131 | TYR | 9.3270 | 0.4323 | 13.7744 |
| BBBB | 132 | GLU | 10.1438 | 0.0000 | 18.2588 |
| BBBB | 133 | ASN | 4.9699 | 1.6521 | 8.2877 |
| BBBB | 134 | HIS | 2.1605 | 0.1201 | 3.5207 |
| BBBB | 135 | ASN | 3.4385 | 4.8744 | 2.0027 |
| BBBB | 136 | ILE | 0.1878 | 0.2608 | 0.1149 |
| BBBB | 137 | SER | 10.4452 | 5.0394 | 21.2570 |
| BBBB | 138 | ILE | 4.4124 | 0.8988 | 7.9260 |
| BBBB | 139 | THR | 13.3105 | 0.5909 | 30.2701 |
| BBBB | 140 | ASN | 6.8155 | 3.1228 | 10.5082 |
| BBBB | 141 | ALA | 1.3398 | 1.6747 | 0.0000 |
| BBBB | 142 | THR | 7.5421 | 0.0097 | 17.5852 |
| BBBB | 143 | VAL | 11.6452 | 1.2806 | 25.4646 |
| BBBB | 144 | GLU | 14.1708 | 1.1173 | 24.6136 |
| BBBB | 145 | ASP | 3.4895 | 0.0584 | 6.9206 |
| BBBB | 146 | SER | 4.2945 | 1.9825 | 8.9184 |
| BBBB | 147 | GLY | 4.3457 | 4.3457 | 0.0000 |
| BBBB | 148 | THR | 4.4415 | 0.0000 | 10.3634 |
| BBBB | 149 | TYR | 0.0214 | 0.0001 | 0.0321 |
| BBBB | 150 | TYR | 3.1691 | 0.0000 | 4.7537 |
| BBBB | 151 | CYS | 0.0000 | 0.0000 | 0.0000 |
| BBBB | 152 | THR | 3.5053 | 0.0000 | 8.1791 |
| BBBB | 153 | GLY | 0.6931 | 0.6931 | 0.0000 |
| BBBB | 154 | LYS | 6.3103 | 0.0180 | 11.3441 |
| BBBB | 155 | VAL | 0.0365 | 0.0044 | 0.0793 |
| BBBB | 156 | TRP | 3.3899 | 5.9121 | 2.3810 |
| BBBB | 157 | GLN | 16.5870 | 5.2100 | 25.6886 |
| BBBB | 158 | LEU | 13.1911 | 0.1922 | 26.1899 |
| BBBB | 159 | ASP | 13.0965 | 5.2133 | 20.9797 |
| BBBB | 160 | TYR | 3.2939 | 3.1627 | 3.3595 |
| BBBB | 161 | GLU | 10.8490 | 4.6790 | 15.7851 |
| BBBB | 162 | SER | 0.5960 | 0.7781 | 0.2318 |
| BBBB | 163 | GLU | 10.5937 | 0.3366 | 18.7993 |
| BBBB | 164 | PRO | 11.6713 | 3.2190 | 22.9411 |
| BBBB | 165 | LEU | 1.9716 | 0.7957 | 3.1476 |
| BBBB | 166 | ASN | 5.2287 | 2.2398 | 8.2176 |
| BBBB | 167 | ILE | 0.2784 | 0.5568 | 0.0000 |
| BBBB | 168 | THR | 9.3922 | 0.0000 | 21.9152 |
| BBBB | 169 | VAL | 0.2895 | 0.5066 | 0.0000 |
| BBBB | 170 | ILE | 9.7952 | 0.6056 | 18.9848 |
| BBBB | 171 | LYS | 14.9992 | 3.9650 | 23.8265 |
| BBBB | 172 | ALA | 8.6682 | 3.2571 | 30.3128 |
| BBBB | 173 | PRO | 17.2332 | 8.4405 | 28.9567 |
| BBBB | 174 | ARG | 24.5074 | 21.0894 | 26.4605 |
| BBBB | 221 | NAG | 17.4850 | 0.0000 | 17.4850 |
| BBBB | 242 | NAG | 10.4355 | 0.0000 | 10.4355 |
| BBBB | 243 | NAG | 10.3502 | 0.0000 | 10.3502 |
| BBBB | 244 | MAN | 15.8885 | 0.0000 | 15.8885 |
| BBBB | 335 | NAG | 8.8279 | 0.0000 | 8.8279 |
| BBBB | 336 | NAG | 16.5384 | 0.0000 | 16.5384 |
| BBBB | 337 | FCA | 16.2107 | 0.0000 | 16.2107 |
| BBBB | 340 | NAG | 13.5916 | 0.0000 | 13.5916 |
| BBBB | 341 | NAG | 21.2819 | 0.0000 | 21.2819 |
| BBBB | 366 | NAG | 21.9238 | 0.0000 | 21.9238 |

TABLE 12
PhFcεRIα₁₋₁₇₂, Form H1, residue exposure
>>>> coordinate set = c703f.pdb

| segid | average accessible area | | | |
|---|---|---|---|---|
| sidechain | resid | resname | residue | mainchain |
| 1 | VAL | 22.5900 | 15.0637 | 32.6251 |
| 2 | PRO | 11.2478 | 3.9295 | 21.0055 |
| 3 | GLN | 15.8860 | 3.9559 | 25.4300 |
| 4 | LYS | 7.8658 | 4.1508 | 10.8378 |
| S | PRO | 0.7859 | 0.7412 | 0.8456 |
| 6 | LYS | 15.0743 | 0.2689 | 26.9185 |
| 7 | VAL | 2.5158 | 4.4026 | 0.0000 |
| 8 | SER | 8.7041 | 1.7476 | 22.6170 |
| 9 | LEU | 3.4804 | 4.2930 | 2.6678 |
| 10 | ASN | 13.3748 | 1.0394 | 25.7103 |
| 11 | PRO | 6.4372 | 0.6223 | 14.1904 |
| 12 | PRO | 9.9906 | 1.9726 | 20.6812 |
| 13 | TRP | 1.6444 | 0.0463 | 2.2837 |
| 14 | ASN | 2.4971 | 0.0178 | 4.9764 |
| 15 | ARG | 1.2172 | 0.0001 | 1.9127 |
| 16 | ILE | 0.3947 | 0.0000 | 0.7895 |
| 17 | PHE | 0.1203 | 0.0000 | 0.1890 |
| 18 | LYS | 9.6134 | 1.5661 | 16.0512 |
| 19 | GLY | 6.4465 | 6.4465 | 0.0000 |
| 20 | GLU | 2.9946 | 0.0000 | 5.3903 |
| 21 | ASN | 4.7501 | 2.8416 | 6.6586 |
| 22 | VAL | 0.3670 | 0.6423 | 0.0000 |
| 23 | THR | 5.0060 | 0.1082 | 11.5364 |
| 24 | LEU | 0.2483 | 0.0000 | 0.4966 |
| 25 | THR | 4.0121 | 0.0000 | 9.3616 |
| 26 | CYS | 0.1821 | 0.1881 | 0.1702 |
| 27 | ASN | 6.6425 | 2.1781 | 11.1069 |
| 28 | GLY | 5.3679 | 5.3679 | 0.0000 |
| 29 | ASN | 17.4099 | 6.2098 | 28.6100 |
| 30 | ASN | 10.2762 | 3.8525 | 16.6998 |
| 31 | PHE | 8.0955 | 2.8330 | 11.1027 |
| 32 | PHE | 13.6377 | 6.1749 | 17.9021 |
| 33 | GLU | 14.0698 | 3.4930 | 22.5313 |
| 34 | VAL | 17.4046 | 4.8614 | 34.1288 |
| 35 | SER | 19.6721 | 12.8131 | 33.3901 |
| 36 | SER | 11.0819 | 4.5899 | 24.0659 |
| 37 | THR | 0.8916 | 0.1902 | 1.8268 |
| 38 | LYS | 8.3803 | 0.0158 | 15.0719 |
| 39 | TRP | 0.0119 | 0.0000 | 0.0167 |
| 40 | PHE | 3.9461 | 0.0636 | 6.1646 |
| 41 | HIS | 3.7169 | 0.6731 | 5.7461 |
| 42 | ASN | 6.6160 | 8.2911 | 4.9408 |
| 43 | GLY | 11.9937 | 11.9937 | 0.0000 |
| 44 | SER | 11.8169 | 1.4187 | 32.6133 |
| 45 | LEU | 12.1877 | 6.2251 | 18.1503 |
| 46 | SER | 4.5272 | 3.1407 | 7.3002 |
| 47 | GLU | 18.3989 | 5.5809 | 28.6533 |
| 48 | GLU | 1.6700 | 0.3535 | 2.7232 |
| 49 | THR | 6.8437 | 0.4232 | 15.4044 |
| 50 | ASN | 6.1820 | 1.4496 | 10.9145 |
| 51 | SER | 8.4271 | 1.0315 | 23.2182 |
| 52 | SER | 6.0403 | 0.9347 | 16.2516 |
| 53 | LEU | 1.9666 | 0.0000 | 3.9331 |
| 54 | ASN | 10.6560 | 4.7155 | 16.5965 |
| 55 | ILE | 1.5407 | 1.0849 | 1.9965 |
| 56 | VAL | 8.4966 | 4.2813 | 14.1170 |
| 57 | ASN | 8.0710 | 0.2994 | 15.8427 |
| 58 | ALA | 0.4475 | 0.5594 | 0.0000 |
| 59 | LYS | 12.6628 | 0.0000 | 22.7931 |
| 60 | PHE | 2.0470 | 0.0207 | 3.2050 |
| 61 | GLU | 9.1096 | 0.1120 | 16.3076 |
| 62 | ASP | 3.9382 | 0.0000 | 7.8765 |
| 63 | SER | 0.1178 | 0.0000 | 0.3535 |
| 64 | GLY | 0.5477 | 0.5477 | 0.0000 |
| 65 | GLU | 3.5925 | 0.1947 | 6.3107 |
| 66 | TYR | 0.3061 | 0.0000 | 0.4592 |
| 67 | LYS | 4.9263 | 0.0000 | 8.8674 |
| 68 | CYS | 0.0002 | 0.0002 | 0.0000 |
| 69 | GLN | 3.1065 | 0.1191 | 5.4965 |
| 70 | HIS | 4.3287 | 0.5494 | 6.8482 |
| 71 | GLN | 14.4511 | 4.6243 | 22.3126 |
| 72 | GLN | 16.7254 | 5.5984 | 25.6271 |
| 73 | VAL | 4.6849 | 0.0239 | 10.8997 |
| 74 | ASN | 3.7390 | 2.2152 | 5.2628 |
| 75 | GLU | 9.8220 | 1.6123 | 16.3897 |
| 76 | SER | 0.9279 | 1.3599 | 0.0638 |
| 77 | GLU | 10.2035 | 0.8100 | 17.7182 |
| 78 | PRO | 6.8952 | 4.7323 | 9.7791 |
| 79 | VAL | 4.4704 | 0.8249 | 9.3311 |
| 80 | TYR | 10.8485 | 1.3619 | 15.5919 |
| 81 | LEU | 0.8740 | 1.0895 | 0.6586 |
| 82 | GLU | 6.2336 | 0.0000 | 11.2205 |
| 83 | VAL | 1.6724 | 2.9266 | 0.0000 |
| 84 | PHE | 3.0301 | 0.8580 | 4.2712 |
| 85 | SER | 10.9935 | 6.5698 | 19.8409 |
| 86 | ASP | 6.6012 | 2.2575 | 10.9449 |
| 87 | TRP | 9.0703 | 0.2059 | 12.6161 |
| 88 | LEU | 0.4451 | 0.8570 | 0.0331 |
| 89 | LEU | 0.5432 | 0.0676 | 1.0187 |
| 90 | LEU | 0.0913 | 0.1298 | 0.0527 |
| 91 | GLN | 0.0763 | 0.0000 | 0.1373 |
| 92 | ALA | 0.0388 | 0.0480 | 0.0022 |
| 93 | SER | 4.5675 | 4.1995 | 5.3034 |
| 94 | ALA | 7.1276 | 1.0786 | 31.3237 |
| 95 | GLU | 6.8795 | 1.2919 | 11.3495 |
| 96 | VAL | 13.0247 | 4.2238 | 24.7593 |
| 97 | VAL | 1.0770 | 0.5985 | 1.7150 |
| 98 | MET | 16.7988 | 0.4914 | 33.1061 |
| 99 | GLU | 7.5393 | 3.3688 | 10.8758 |
| 100 | GLY | 3.1157 | 3.1157 | 0.0000 |
| 101 | GLN | 10.1587 | 0.2304 | 18.1014 |
| 102 | PRO | 8.7856 | 1.4883 | 18.5154 |
| 103 | LEU | 0.0405 | 0.0000 | 0.0810 |
| 104 | PHE | 5.7390 | 0.0000 | 9.0184 |
| 105 | LEU | 0.0000 | 0.0000 | 0.0000 |
| 106 | ARG | 4.9770 | 0.0000 | 7.8210 |
| 107 | CYS | 2.8329 | 3.8594 | 0.7800 |
| 108 | HIS | 1.0226 | 0.3429 | 1.4757 |
| 109 | GLY | 0.7524 | 0.7524 | 0.0000 |
| 110 | TRP | 4.3881 | 0.0000 | 6.1433 |
| 111 | ARG | 13.1221 | 5.0820 | 17.7164 |
| 112 | ASN | 12.3893 | 5.7597 | 19.0188 |
| 113 | TRP | 6.4754 | 2.8590 | 7.9219 |
| 114 | ASP | 11.2956 | 2.1441 | 20.4471 |
| 115 | VAL | 2.0499 | 2.1826 | 1.8731 |
| 116 | TYR | 11.1258 | 1.0112 | 16.1831 |
| 117 | LYS | 16.7863 | 4.7622 | 26.4055 |
| 118 | VAL | 8.1424 | 6.0958 | 10.8711 |
| 119 | ILE | 6.8012 | 0.9964 | 12.6060 |
| 120 | TYR | 2.8442 | 0.9061 | 3.8133 |
| 121 | TYR | 3.5867 | 0.0012 | 5.3794 |
| 122 | LYS | 5.1214 | 0.6012 | 8.7376 |
| 123 | ASP | 7.4941 | 4.6376 | 10.3507 |
| 124 | GLY | 12.2128 | 12.2128 | 0.0000 |
| 125 | GLU | 15.1128 | 1.2362 | 26.2141 |
| 126 | ALA | 11.6923 | 3.6139 | 44.0058 |
| 127 | LEU | 4.6471 | 5.4019 | 3.8923 |
| 128 | LYS | 18.8922 | 6.6649 | 28.6740 |
| 129 | TYR | 17.4834 | 7.0989 | 22.6757 |
| 130 | TRP | 2.4961 | 4.3542 | 1.7528 |
| 131 | TYR | 12.7233 | 5.2485 | 16.4608 |
| 132 | GLU | 13.6861 | 0.7556 | 23.9944 |
| 133 | ASN | 9.3922 | 6.3761 | 12.4084 |
| 134 | HIS | 15.2795 | 8.2917 | 19.9381 |
| 135 | ASN | 11.1940 | 2.7459 | 19.6420 |
| 136 | ILE | 5.4540 | 0.6821 | 10.2259 |
| 137 | SER | 0.6915 | 0.0000 | 2.0744 |
| 138 | ILE | 6.3883 | 1.6352 | 11.1413 |
| 139 | THR | 4.7987 | 2.2363 | 8.2152 |
| 140 | ASN | 5.2615 | 2.7779 | 7.7451 |
| 141 | ALA | 0.9545 | 1.1931 | 0.0000 |
| 142 | THR | 6.9219 | 0.6069 | 15.3420 |
| 143 | VAL | 9.5663 | 1.8575 | 19.8448 |
| 144 | GLU | 14.4371 | 3.2860 | 23.3579 |
| 145 | ASP | 2.2220 | 0.0056 | 4.4384 |
| 146 | SER | 5.2393 | 2.1712 | 11.3756 |
| 147 | GLY | 3.0536 | 3.0536 | 0.0000 |
| 148 | THR | 2.8393 | 0.0000 | 6.6250 |
| 149 | TYR | 0.0489 | 0.0000 | 0.0734 |
| 150 | TYR | 3.3061 | 0.0181 | 4.9500 |
| 151 | CYS | 0.0000 | 0.0000 | 0.0000 |
| 152 | THR | 3.7148 | 0.0000 | 8.6679 |
| 153 | GLY | 0.9412 | 0.9412 | 0.0000 |
| 154 | LYS | 8.4275 | 0.1238 | 15.0704 |
| 155 | VAL | 0.3174 | 0.0138 | 0.7223 |
| 156 | TRP | 14.1884 | 7.2900 | 16.9477 |
| 157 | GLN | 15.4584 | 4.5161 | 24.2122 |
| 158 | LEU | 11.5234 | 0.7276 | 22.3193 |
| 159 | ASP | 15.3714 | 7.3517 | 23.3910 |
| 160 | TYR | 4.5849 | 1.8192 | 5.9678 |
| 161 | GLU | 12.0528 | 4.8779 | 17.7927 |
| 162 | SER | 0.9680 | 1.3220 | 0.2601 |
| 163 | GLU | 10.9265 | 0.9944 | 18.8722 |
| 164 | PRO | 11.4182 | 2.7638 | 22.9575 |
| 165 | LEU | 1.8797 | 0.4866 | 3.2727 |
| 166 | ASN | 5.1428 | 2.5621 | 7.7235 |
| 167 | ILE | 0.3717 | 0.7433 | 0.0000 |
| 168 | THR | 9.9155 | 0.0000 | 23.1363 |
| 169 | VAL | 0.2713 | 0.4747 | 0.0000 |
| 170 | ILE | 12.6290 | 1.6535 | 23.6046 |
| 171 | LYS | 18.1223 | 11.6928 | 23.2660 |
| 221 | NAG | 10.3807 | 0.0000 | 10.3807 |
| 222 | NAG | 20.2927 | 0.0000 | 20.2927 |
| 242 | NAG | 10.3379 | 0.0000 | 10.3379 |
| 243 | NAG | 10.0051 | 0.0000 | 10.0051 |
| 244 | MAN | 17.1981 | 0.0000 | 17.1981 |
| 250 | NAG | 15.4600 | 0.0000 | 15.4600 |
| 274 | NAG | 20.0516 | 0.0000 | 20.0516 |
| 340 | NAG | 16.0149 | 0.0000 | 16.0149 |
| 341 | NAG | 20.8951 | 0.0000 | 20.8951 |
| 366 | NAG | 14.4348 | 0.0000 | 14.4348 |
| 367 | NAG | 20.6913 | 0.0000 | 20.6913 |

TABLE 13
Crystallographic data and model refinement

| Data Set | Form H1 | Fomr M2 | Form T1 | Form T2 |
|---|---|---|---|---|
| res. | 3.2 | 3.2 | 3.1 | 3.8 |
| wavel(A) | 1.0039 | 1.0047 | 0.914 | 0.92 |
| comp/(last shell) | 93.2(95.4) | 99.3(100) | 97.0(83.3) | 85.7(80.2) |
| av. red. | 8.0(7.1) | 4.1(4.2) | 7.3(2.2) | 2.0(1.6) |
| (last shell) | | | | |
| Rmerge | 10.3(51.6) | 9.7(43.6) | 11.2(76.6) | 6.3(60.9) |
| (last shell) | | | | |
| l/sigl (last shell) | 11.8(5.3) | 9.2(3.0) | 7.9(1.1) | 7.5(1.1) |
| # refl(free) | 4030(412) | 11640(620) | 23318(1180) | 14239(740) |
| Rfactor/Rfree | 28.8/31.3 | 25.4/28.3 | 29.1/32.9 | 27.8/30.4 |
| # atoms | 1537 | 3120 | 7660 | 7660 |
| # waters | 0 | 0 | 0 | 0 |
| RMSD bonds | 0.0084 | 0.0096 | 0.0100 | 0.0086 |
| RMSD angles | 1.53 | 1.60 | 1.50 | 1.40 |
| Ave. B | 97.1 | 69.4 | 137.6 | 191.1 |

TABLE 14
Root mean square deviations for alpha carbon positions

| | RMSB v. | | |
|---|---|---|---|
| | Orig | | |
| model: | (Angs) | # CA | segments |
| H1 | 0.855 | 155 | 4-27/28-31/36-70/73-129/137-171 |
| H1 30 loop | 3.667 | 6 | 27-31, 36 |
| H1 130 loop | 4.176 | 9 | 129-137 |
| M2 copy A | 0.680 | 157 | 4-27/36-130/134-171 |
| M2A 30 loop | 5.212 | 6 | 27-31, 36 |
| M2A 130 loop | 3.818 | 5 | 130-134 |
| M2 copy B | 0.766 | 155 | 4-27/36-127/133-171 |
| M2B 30 loop | 4.258 | 6 | 27-31, 36 |
| M2B 130 loop | 6.938 | 7 | 127-133 |
| T1 copy C | 0.839 | 155 | 4-28/36-71/73-127/133-171 |
| T1C 30 loop | 6.372 | 5 | 28-31, 36 |
| T1C 130 loop | 7.449 | 7 | 127-133 |
| T2 copy C | 0.867 | 155 | 4-28/36-71/73-127/133-171 |
| T1C 30 loop | 6.319 | 5 | 28-31, 36 |
| T1C 130 loop | 7.476 | 7 | 127-133 |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A three-dimensional model selected from the group consisting of: (a) a three-dimensional model of an extracellular domain of a human high affinity Fc epsilon receptor alpha chain (FcεRIα) protein, wherein said model substantially represents the atomic coordinates specified in a table selected from the group consisting of Table 1, Table 5, Table 6, Table 7, and Table 8; and (b) a three-dimensional model comprising a modification of said model of (a), wherein said modification represents a protein that binds to a Fc domain of an antibody.

2. The model of claim 1, wherein said model is represented by a method selected from the group consisting of listing the coordinates of all atoms comprising said model, providing a physical three-dimensional model, imaging said model on a computer screen, providing a picture of said model, and deriving a set of coordinates based of a picture of said model.

3. The model of claim 1, wherein said model identifies the solvent accessibility of amino acid residues of said protein listed in a table selected from the group consisting of Table 2, Table 9, Table 10, Table 11 and Table 12.

4. The model of claim 1, wherein said model represents a protein that binds to a Fc domain of an IgE antibody with an affinity that is at least equivalent to the affinity of the extracellular domain of human FcεRIα for an IgE antibody selected from the group consisting of a human IgE antibody, a canine IgE antibody, a feline IgE antibody, an equine IgE antibody, a rat IgE antibody, and a murine IgE antibody.

5. The model of claim 1, wherein said model represents a protein that selectively binds to a mammalian antibody selected from the group consisting of an IgE antibody and an IgG antibody.

6. The model of claim 1, wherein said model represents an extracellular domain of a protein selected from the group consisting of a human FcεRIα protein, a canine FcεRIα protein, a feline FcεRIα protein, an equine FcεRIα protein, a murine FcεRIα protein, and a rat FcεRIα protein.

7. The model of claim 1, wherein said model comprises a three-dimensional model of an extracellular antibody binding domain of an antibody receptor protein other than human FcεRIα.

8. The model of claim 7, wherein said model is produced by incorporating all or any part of the amino acid sequence of said other antibody receptor protein into a three-dimensional model of said extracellular domain of said human FcεRIα protein to produce said model of said other antibody receptor protein.

9. The model of claim 1, wherein said model represents an IgE binding domain.

10. The model of claim 1, wherein said model is produced by a method comprising:
(a) crystallizing an extracellular domain of a human FcεRIα protein;
(b) collecting X-ray diffraction data from said crystallized protein; and
(c) determining said model from said data and amino acid sequence of said protein.

11. The model of claim 10, wherein said protein has an amino acid sequence selected from the group consisting of SEQ ID NO: 2, SEQ ID NO: 4 and SEQ ID NO: 4 except that the isoleucine at position 170 is replaced with a cysteine.

12. The model of claim 1, wherein said model has a three-dimensional structure comprising atomic coordinates that have a root mean square deviation of protein backbone atoms of less than 10 angstroms when superimposed on said three-dimensional model substantially represented by the atomic coordinates specified in a table selected from the group consisting of Table 1, Table 5, Table 6, Table 7, and Table 8.

13. The model of claim 1, wherein said modification has an amino acid sequence that shares at least about 30% amino acid sequence homology with a FcεRIα protein having an amino acid sequence selected from the group consisting of SEQ ID NO: 2 and SEQ ID NO: 4.

14. The model of claim 1, wherein said model represents a FcεRIα protein having increased stability compared to the stability of a human FcεRIα protein having an amino acid sequence selected from the group consisting of SEQ ID NO: 2 and SEQ ID NO: 4.

15. The model of claim 1, wherein said model represents a FcεRIα protein having increased affinity for IgE compared to the affinity of a human FcεRIα protein having an amino acid sequence selected from the group consisting of SEQ ID NO: 2 and SEQ ID NO: 4 for IgE.

16. The model of claim 1, wherein said model represents a FcεRIα protein having altered substrate affinity compared to the affinity of a human FcεRIα protein having an amino acid sequence selected from the group consisting of SEQ ID NO: 2 and SEQ ID NO: 4 for IgE.

17. The model of claim 1, wherein said model comprises a three-dimensional model of a FcεRIα protein having increased solubility compared to the solubility of a human FcεRIα protein having an amino acid sequence selected from the group consisting of SEQ ID NO: 2 and SEQ ID NO: 4.

18. The model of claim 1, wherein said model is used to identify an inhibitor of the selective binding between a FcεRIα protein and an IgE antibody.

19. The model of claim 1, wherein said model identifies crystal contacts between a FcεRIα protein and a Fc domain of an IgE antibody.

20. The model of claim 1, wherein domain 1 and domain 2 are oriented in a manner as specified by the structural coordinates specified in a table selected from the group consisting of Table 1, Table 5, Table 6, Table 7 and Table 8.

21. The model of claim 1, wherein said model identifies amino acids in the D1D2 interface.

22. A method to produce a three-dimensional model of an extracellular domain of a human FcεRIα protein, said method comprising representing amino acids of said protein at substantially the atomic coordinates specified in a table selected from the group consisting of Table 1, Table 5, Table 6, Table 7 and Table 8.

23. The method of claim 22, wherein said model is represented by a method selected from the group consisting of listing the coordinates of all atoms comprising said model, providing a physical three-dimensional model, imaging said model on a computer screen, providing a picture of said model, and deriving a set of coordinates based of a picture of said model.

24. A method to produce a three-dimensional model of an antibody receptor protein other than a human FcεRIα protein represented by the three-dimensional model substantially representing the atomic coordinates specified in a table selected from the group consisting of Table 1, Table 5, Table 6, Table 7 and Table 8, said method comprising homology modeling.

25. The method of claim 24, wherein said method of homology modeling comprises incorporating at least a portion of the amino acid sequence of said other antibody receptor protein into said three-dimensional model substantially representing the atomic coordinates specified in a table selected from the group consisting of Table 1, Table 5, Table 6, Table 7 and Table 8 to produce said model of said other antibody receptor protein.

26. The method of claim 24, wherein said method comprises orienting two immunoglobulin domains in an orientation as represented by the D1 and D2 domains of said model substantially representing the atomic coordinates specified in a table selected from the group consisting of Table 1, Table 5, Table 6, Table 7 and Table 8.

27. An isolated crystal of an extracellular domain of a FcεRIα protein.

28. The crystal of claim 27, wherein said protein has an amino acid sequence selected from the group consisting of SEQ ID NO: 2, SEQ ID NO: 4, and SEQ ID NO: 4 except that the isoleucine at position 170 is replaced with a cysteine.

29. The crystal of claim 27, wherein said crystal belongs to a space group selected from the group consisting of monoclinic space group C2, hexagonal space group P6₁22, and tetragonal space group P4₃.

30. The crystal of claim 29, wherein said crystal of monoclinic space group C2 has cell dimensions selected from the group consisting of: 88.6 angstroms×69.6 angstroms×49.3 angstroms, alpha=gamma=90.0 degrees, beta=116.69 degrees; 136.02 angstroms×75.01 angstroms×79.28 angstroms, alpha=gamma=90 degrees, beta=117.8 degrees; and 136.90 angstroms×73.79 angstroms×79.40 angstroms, alpha=gamma=90 degrees, beta=117.74 degrees, and wherein said tetragonal space group P4₃ has cell dimensions selected from the group consisting of: 145.08 angstroms×145.08 angstroms×62.74 angstroms, alpha=beta=gamma=90 degrees; and 150.50 angstroms×150.50 angstroms×74.18 angstroms, alpha=beta=gamma=90 degrees.

31. The crystal of claim 29, wherein said crystal of hexagonal space group P6₁22 has cell dimensions selected from the group consisting of: 58 angstroms×58 angstroms×226 angstroms, alpha=beta=90 degrees, gamma=120 degrees; and 58.62 angstroms×58.62 angstroms×229.19 angstroms, alpha=beta=90 degrees, gamma=120 degrees.

32. The crystal of claim 27, wherein said protein is produced in insect cells or Chinese hamster ovary cells.

33. The crystal of claim 27, wherein said crystal diffracts X-rays to a resolution selected from the group consisting of about 2.4 angstroms, about 3.1 angstroms, about 3.2 angstroms, and about 3.8 angstroms.

34. A method to produce an isolated crystal of an extracellular domain of a FcεRIα protein, said method comprising vapor diffusion.

35. The method of claim 34, wherein said protein has an amino acid sequence selected from the group consisting of SEQ ID NO: 2, SEQ ID NO: 4, and SEQ ID NO: 4 except that the isoleucine at position 170 is replaced by a cysteine.

36. The method of claim 34, wherein said crystal is selected from the group consisting of: a monoclinic space group C2 having cell dimensions of 88.6 angstroms×69.6 angstroms×49.3 angstroms, alpha=gamma=90.0 degrees, beta=116.69 degrees; a monoclinic space group C2 having cell dimensions of 136.02 angstroms×75.01 angstroms×79.28 angstroms, alpha=gamma=90 degrees, beta=117.8 degrees; a monoclinic space group C2 having cell dimensions of 136.90 angstroms×73.79 angstroms×79.40 angstroms, alpha=gamma=90 degrees, beta=117.74 degrees; a tetragonal space group P4₃ having cell dimensions of 145.08 angstroms×145.08 angstroms×62.74 angstroms, alpha=beta=gamma=90 degrees; a tetragonal space group P4₃ having cell dimensions of 150.50 angstroms×150.50 angstroms×74.18 angstroms, alpha=beta=gamma=90 degrees; a hexagonal space group P6₁22 having cell dimensions of 58 angstroms×58 angstroms×226 angstroms, alpha=beta=90 degrees, gamma=120 degrees; and a hexagonal space group P6₁22 having cell dimensions of 58.62 angstroms×58.62 angstroms×229.19 angstroms, alpha=beta=90 degrees, gamma=120 degrees.

37. The method of claim 34, wherein said protein is produced in insect cells or Chinese hamster ovary cells.

38. The method of claim 34, wherein said crystal diffracts X-rays to a resolution selected from the group consisting of about 2.4 angstroms, about 3.1 angstroms, about 3.2 angstroms, and about 3.8 angstroms.

39. An isolated FcεRIα protein selected from the group consisting of: (a) a protein consisting of SEQ ID NO: 2; (b) a protein consisting of SEQ ID NO: 4 except that the isoleucine at position 170 is replaced with a cysteine; and (c) a protein that is structurally homologous to a protein of (a) or (b), wherein said protein of (c) binds to a Fc domain of an antibody.

40. The protein of claim 39, wherein said protein is produced in insect cells or Chinese hamster ovary cells.

41. The protein of claim 39, wherein said FcεRIα protein is selected from the group consisting of a human FcεRIα protein, a feline FcεRIα protein, a canine FcεRIα protein, an equine FcεRIα protein, a murine FcεRIα protein, and a rat FcεRIα protein.

42. A nucleic acid molecule comprising a nucleic acid sequence that encodes said protein of claim 39.

43. A recombinant molecule comprising a nucleic acid sequence of claim 42.

44. A recombinant virus comprising a nucleic acid sequence of claim 42.

45. A recombinant cell comprising a nucleic acid sequence of claim 42.

46. A method to produce a protein comprising culturing a recombinant cell of claim 45.

47. A method to identify a compound that inhibits the binding between an IgE antibody and a FcεRIα protein, said method comprising using a three-dimensional model of an extracellular domain of a human high affinity FcεRIα protein to identify said compound, wherein said model substantially represents the atomic coordinates specified in a table selected from the group consisting of Table 1, Table 5, Table 6, Table 7 and Table 8.

48. The method of claim 47, wherein said compound interacts with a region of said model selected from the group consisting of the IgE binding domain, the D1D2 interface, and the cleft between domain 1 and domain 2.

49. The method of claim 47, wherein said compound interacts with a region of said model selected from the group consisting of a A′B loop of domain 1, a EF loop of domain 1, a BC loop of domain 2, a C strand of domain 2, a CC′ loop of domain 2, a C′E loop of domain 2, a F strand of domain 2, a FG loop of domain 2, and a tryptophan-containing hydrophobic ridge.

50. The method of claim 47, wherein said compound interacts with a region of said model in which N-linked glycosylation sites are absent.

51. The method of claim 47, wherein said compound interacts with an amino acid selected from the group consisting of: (a) a residue having a position in SEQ ID NO: 2 or SEQ ID NO: 4 selected from the group consisting of position 87, 115, 117, 118, 120-123, 128, 129, 131, 149, 153, 155 and 159; and (b) a surface residue within about 10 angstroms of any of said residues of (a).

52. The method of claim 47, wherein said compound interacts with an amino acid selected from the group consisting of: (a) a residue having a position in SEQ ID NO: 2 or SEQ ID NO: 4 selected from the group consisting of position 87, 117, 121, 123, 128, and 159; and (b) a surface residue within about 10 angstroms of any of said residues of (a).

53. The method of claim 47, wherein said method comprises:
(a) generating said model, or a model of an IgE binding domain thereof, on a computer screen;
(b) generating the spacial structure of a compound to be tested; and
(c) testing to determine if said compound interacts with said IgE binding domain, wherein such an interaction indicates that said compound is capable of inhibiting said binding of an IgE antibody to a FcεRIα protein.

54. The method of claim 53, wherein said step (a) includes the step of identifying one or more amino acid(s) in the IgE binding domain of said model that interact directly with the Fc domain of an IgE antibody when said Fc domain binds to said IgE binding domain.

55. The method of claim 54, wherein said compound interacts directly with one or more of said amino acid(s).

56. An inhibitory compound identified in accordance with the method of claim 47.

57. A therapeutic composition comprising an inhibitory compound of claim 56.

58. A method to protect an animal from allergy, said method comprising administering to said animal an inhibitory compound of claim 56.

59. A mutein that binds to a Fc domain of an antibody, wherein said mutein has an improved function compared to a protein comprising an amino acid sequence selected from the group consisting of SEQ ID NO: 2 and SEQ ID NO: 4, wherein said improved function is selected from the group consisting of increased stability, increased affinity for an Fc domain of an antibody, altered substrate specificity, and increased solubility, wherein said mutein is produced by a method comprising:
(a) analyzing a three-dimensional model substantially representing the atomic coordinates specified in a table selected from the group consisting of Table 1, Table 5, Table 6, Table 7 and Table 8 to identify at least one amino acid of the protein represented by said model which if replaced by a specified amino acid would effect said improved function of said protein; and
(b) replacing said identified amino acid(s) to produce said mutein having said improved function.

60. The mutein of claim 59, wherein said step of replacing does not substantially disrupt the three-dimensional structure of said protein.

61. The mutein of claim 59, wherein said mutein has an increased stability compared to an unmodified antibody receptor protein.

62. The mutein of claim 59, wherein said mutein has an increased shelf-life compared to an unmodified antibody receptor protein.

63. The mutein of claim 59, wherein said mutein has a K_{A} for said Fc domain of at least about 3×10⁹ liters/mole.

64. The mutein of claim 59, wherein said mutein has a kₐ for said Fc domain of at least about 1×10⁵ liters/mole-second.

65. The mutein of claim 59, wherein said mutein has a k_{d} for said Fc domain of less than or equal to 3×10⁻⁵/second.

66. The mutein of claim 59, wherein said antibody is an IgE antibody.

67. The mutein of claim 59, wherein said mutein is produced by a method comprising:
(a) comparing the IgE binding domain on said model with amino acid sequence of an antibody receptor protein with an improved function to identify at least one amino acid segment of said antibody receptor protein with said improved function that if incorporated into said FcεRIα protein represented by said model would give said FcεRIα protein said improved function; and
(b) incorporating said segment into said FcεRIα protein, thereby producing a mutein with said improved function.

68. The mutein of claim 59, wherein said mutein is produced by a method comprising:
(a) using said model to identify a three-dimensional arrangement of residues that can be randomized by mutagenesis to allow the construction of a library of molecules from which an improved function can be selected; and
(b) identifying at least one member of said mutagenized library having said improved function.

69. The mutein of claim 59, wherein said mutein is produced by a method comprising:
(a) effecting random mutagenesis of nucleic acid molecules encoding a target of a FcεRIα protein as identified by analyzing a model of that protein;
(b) cloning said mutagenized nucleic acid molecules into a phage display library, wherein said phage display library expresses said target; and
(c) identifying at least one member of the library that expresses said target, said target having an improved function.

70. The mutein of claim 69, wherein said target comprises an IgE binding domain and wherein said improved function comprises increased affinity of said domain for an antibody.

71. The mutein of claim 59, wherein said step of replacing is selected from the group consisting of:
(a) replacing at least one amino acid in at least one non-constrained loop of domain 1 in an area proximal to the FceRI gamma chain putative binding site;
(b) joining an amino-terminal amino acid residue to a carboxyl-terminal amino acid residue of an extracellular domain of a FcεRIα protein;
(c) replacing at least one amino acid site with an amino acid suitable for derivatization;
(d) replacing at least one pair of amino acids of said protein with a cysteine pair to enable the formation of a disulfide bond that stabilizes said mutein;
(e) removing at least a portion of the region between the B strand and C strand of domain 1;
(f) removing at least a portion of the region between the C strand and E strand of domain 1;
(g) replacing at least one amino acid in the IgE binding domain in order to increase the affinity between an IgE antibody and said protein;
(h) replacing at least one amino acid of said protein with an amino acid such that said replacement decreases the entropy of unfolding of said protein;
(i) replacing at least one amino acid of said protein selected from the group consisting of asparagines and glutamines with an amino acid that is less susceptible to deamidation than is said amino acid to be replaced;
(j) replacing at least one amino acid of said protein selected from the group consisting of methionines, histidines and tryptophans with an amino acid that is less susceptible to an oxidation or reduction reaction than is said amino acid to be replaced;
(k) replacing at least one arginine of said protein with an amino acid that is less susceptible to dicarbonyl compound modification than is said amino acid to be replaced;
(l) replacing at least one amino acid of said protein susceptible to reaction with a reducing sugar sufficient to reduce said protein function with an amino acid less susceptible to said reaction;
(m) replacing at least one amino acid of said protein with an amino acid capable of increasing the stability of the inner core of said protein;
(n) replacing at least one amino acid of said protein with at least one N-linked glycosylation site;
(o) replacing at least one N-linked glycosylation site of said protein with at least one amino acid that does not comprise an N-linked glycosylation site; and
(p) replacing at least one amino acid of said protein with an amino acid that reduces aggregation of said protein.

72. The mutein of claim 59, further comprising a substance attached to an amino acid of said mutein such that said substance does not substantially interfere with the antibody binding activity of said protein.

73. An isolated nucleic acid sequence encoding a mutein of claim 59.

74. A recombinant molecule comprising said nucleic acid sequence of claim 73 operatively linked to an expression vector.

75. A recombinant virus comprising said nucleic acid sequence of claim 73.

76. A recombinant cell comprising said nucleic acid sequence of claim 73, wherein said cell is capable of expressing said nucleic acid sequence.

77. A method to produce a mutein comprising culturing a recombinant cell of claim 76.

78. A diagnostic reagent comprising a mutein of claim 59.

79. A therapeutic composition comprising a mutein of claim 59.

80. A method to use a mutein of claim 59, wherein said method is selected from the group consisting of: (a) a method to protect an animal from allergy, said method comprising administering a therapeutic composition comprising said mutein to said animal; (b) a method to detect allergy, or susceptibility thereto, in an animal, said method comprising using said mutein to detect said allergy; and (c) a method to enhance the performance of an IgE binding assay, said method comprising incorporating into said assay said mutein.

81. A mutein having an improved function compared to an unmodified FcεRIα protein, wherein said improved function is selected from the group consisting of increased stability, increased affinity for an Fc domain of an antibody, altered substrate specificity, and increased solubility, wherein the amino acid sequence of said mutein differs in at least one position from the amino acid sequence of said unmodified protein, said position being in a region selected from the group consisting of a crystal contact cluster, a tryptophan-containing hydrophobic ridge, a FG loop in D2, a D1D2 interface, a cleft between D1 and D2, a domain 1, a domain 2, a hydrophobic core, a A′B loop of D1, a EF loop of D1, a BC loop of D2, a C strand of D2, a CC′ loop of D2, a C′E loop of D2, a strand of D2, the amino terminal five residues of said protein, and the carboxyl terminal five residues of said protein.

82. A method to improve a function of a FcεRIα protein, said improved function being selected from the group consisting of increased stability, increased affinity for an Fc domain of an antibody, altered substrate specificity, and increased solubility, said method comprising:
(a) analyzing a three-dimensional model of an extracellular domain of a human high affinity FcεRIα protein substantially representing the atomic coordinates specified in a table selected from the group consisting of Table 1, Table 5, Table 6, Table 7 and Table 8 to identify at least one amino acid of said protein which if replaced by a specified amino acid improves at least one of said functions of said protein; and
(b) replacing said identified amino acid(s) to produce a mutein having at least one of said improved functions.

83. An isolated FcεRIα protein selected from the group consisting of: a crystal contact cluster involved in IgE binding; a tryptophan-containing hydrophobic ridge; a FG loop in D2; a D1D2 interface; a cleft between D1 and D2; a domain 1; a domain 2; a hydrophobic core; a A′B loop of D1; a EF loop of D1; a BC loop of D2; a C strand of D2; a CC′ loop of D2; a C′E loop of D2; and a strand of D2.

84. An isolated nucleic acid molecule encoding a protein of claim 83.
